(19) United States
(12) Patent Application Publication
(10) Pub. No.: US 2025/0368649 A1
(43) Pub. Date: Dec. 4, 2025

(54) TETRAHYDROPYRIDOPYRIMIDINE PAN-KRAS INHIBITORS

(71) Applicant: Mirati Therapeutics, Inc., San Diego, CA (US)

(72) Inventors: Xiaolun WANG, San Diego, CA (US); Anthony IVETAC, San Diego, CA (US); Svitlana KULYK, San Diego, CA (US); John David LAWSON, Carlsbad, CA (US); Matthew Arnold MARX, San Diego, CA (US); Christopher Ronald SMITH, San Diego, CA (US)

(21) Appl. No.: 18/875,603

(22) PCT Filed: Jun. 13, 2023
(86) PCT No.: PCT/US2023/025200

§ 371 Date: Dec. 16, 2024

Related U.S. Application Data
(60) Provisional application No. 63/352,192, filed on Jun. 14, 2022.
(60) Provisional application No. 63/438,703, filed on Jan. 12, 2023.
(60) Provisional application No. 63/459,551, filed on Apr. 14, 2023.

(51) Int. Cl. C07D 487/04; A61K 31/519; A61K 31/551; C07D 471/04; C07D 471/10; C07D 487/08; C07D 487/10; C07D 491/107; C07D 498/08; C07D 498/10; C07D 513/04; C07D 513/10

(52) U.S. Cl. CPC C07D 487/04; A61K 31/519; A61K 31/551; C07D 471/04; C07D 471/10; C07D 487/08; C07D 487/10; C07D 491/107; C07D 498/08; C07D 498/10; C07D 513/04; C07D 513/10

(57) ABSTRACT

The present invention relates to compounds that inhibit at least one of KRas wild type, KRas G12A, KRas G12C, KRas G12D, KRas G12R, KRas G12S, KRas G12V, KRas G13D and KRas Q61H, pharmaceutical compositions comprising the compounds and methods of use therefor.

### FIELD OF THE INVENTION

The present invention relates to compounds that inhibit multiple mutated forms of KRas, i.e., pan-KRas inhibitors. In particular, the present invention relates to pan-KRas compounds, pharmaceutical compositions comprising the compounds and methods of use therefor.

### BACKGROUND OF THE INVENTION

Kirsten Rat Sarcoma 2 Viral Oncogene Homolog (“KRas”) is a small GTPase and a member of the Ras family of oncogenes. KRas serves as a molecular switch cycling between inactive (GDP-bound) and active (GTP-bound) states to transduce upstream cellular signals received from multiple tyrosine kinases to downstream effectors to regulate a wide variety of processes, including cellular proliferation (e.g., see Alamgeer et al., (2013) Current Opin Pharmcol. 13:394-401).

The role of activated KRas in malignancy was observed over thirty years ago (e.g., see Santos et al., (1984) Science 223:661-664). Aberrant expression of KRas accounts for up to 20% of all cancers and oncogenic KRas mutations that stabilize GTP binding and lead to constitutive activation of KRas. KRas mutations at codons 12, 13, 61 and other positions of the KRas primary amino acid sequence are present in 88% of all pancreatic adenocarcinoma patients, 50% of all colon/rectal adenocarcinoma patients, and 32% lung adenocarcinoma patients (e.g., see Prior et all., (2020) Cancer Res 80:2969-74). A recent publication also suggested wild type Kras inhibition could be a viable therapeutic strategy to treat KRas^{WT} dependent cancers (e.g., see Bery et al., (2020) Nat. Commun. 11: 3233).

The well-known role of KRas in malignancy and the discovery of these frequent mutations in KRas in various tumor types made KRas a highly attractive target of the pharmaceutical industry for cancer therapy. Notwithstanding thirty years of large-scale discovery efforts to develop inhibitors of KRas for treating cancer, no KRas inhibitor has yet demonstrated sufficient safety and/or efficacy to obtain regulatory approval (e.g., see McCormick (2015) Clin Cancer Res. 21 (8):1797-1801).

Compounds that inhibit KRas activity are still highly desirable and under investigation, including those that disrupt effectors such as guanine nucleotide exchange factors (e.g., see Sun et al., (2012) Agnew Chem Int Ed Engl. 51(25):6140-6143 doi: 10.1002/anie201201358) as well recent advances in the covalent targeting of an allosteric pocket of KRas G12C (e.g., see Ostrem et al., (2013) Nature 503:548-551 and Fell et al., (2018) ACS Med. Chem. Lett. 9:1230-1234). Clearly there remains a continued interest and effort to develop inhibitors of KRas, particularly inhibitors of activating KRas mutants.

Thus, there is a need to develop new pan-KRas inhibitors that demonstrate sufficient efficacy for treating KRas-mediated cancers.

### SUMMARY OF THE INVENTION

In one aspect of the invention, compounds are provided that inhibit KRas activity.

In one aspect of the invention, there is provided a compound of Formula (I):

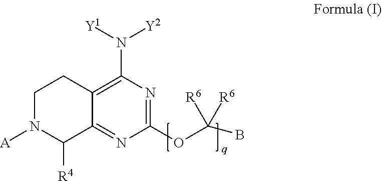

or a pharmaceutically acceptable salt thereof, wherein:
A is aryl or heteroaryl, wherein the aryl or the heteroaryl is optionally substituted with 1-4 R¹;
B is selected from:

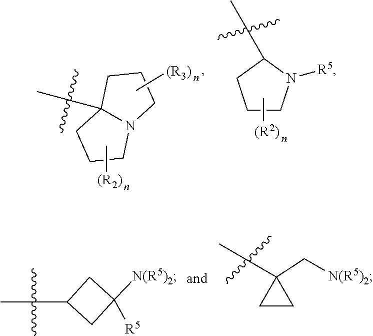

Y¹ is L-hydrogen optionally substituted with 1-4 R⁸, hydroxy, halogen, L—C3-C6 cycloalkyl optionally substituted with 1-4 R⁹, L—S(O)₂N(R⁵)₂ optionally substituted with 1-4 R⁹, L-heteroaryl optionally substituted with 1-4 R⁸, L-aryl optionally substituted with 1-4 R⁸, and L-heterocycle substituted with 1-2 oxo (═O) or oxo-containing substituent and optionally further substituted with 1-2 heteroaryl-R⁸ or R⁸;
Y² is hydrogen or C1-C4 alkyl;
or Y and Y² join to form:

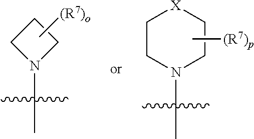

where X is selected from: a bond, —S—, —O—, —N<bound to a fused ring, —CH₂—, —CH₂—NH—, —CH₂—NH—CH₂—, —CH₂—CH₂—CH₂—, —CH₂—CH₂—, —O—CH₂— and —S—CH₂—;
each R¹ is independently halogen, cyano, hydroxy, C1-C4 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, —S—C1-C3 haloalkyl, C1-C3 alkoxy, hydroxyC1-C3 alkyl, —CH₂C(═O)N(R⁵)₂, —C3-C4 alkynyl(NR⁵)₂, —N(R⁵)₂, deuteroC2-C4 alkynyl, (C1-C3 alkoxy)haloC1-C3 alkyl-, or C3-C6 cycloalkyl wherein said C3-C6 cycloalkyl is optionally substituted with halogen or C1-C3 alkyl;
each R² is independently hydrogen, hydroxy, halogen, C1-C3 alkyl, C1-C3 cyanoalkyl, C1-C3 hydroxyalkyl, HC(═O)—, —OC(O)N(R⁵)₂, —CH₂OC(O)N(R⁵)₂, —CH₂NR⁵—SO₂—N(R⁵)₂, —CO₂R⁵, —CO₂N(R⁵)₂, ═CH₂, ═CHR″ or ═C(R″)₂;
each R³ is independently hydrogen, hydroxy, halogen, L—C1-C3 alkyl, C1-C3 cyanoalkyl, C1-C3 hydroxyalkyl, HC(═O)—, —OC(O)N(R⁵)₂, —CH₂OC(O)N(R⁵)₂, —CH₂NR⁵—SO₂—N(R⁵)₂, —CO₂R⁵, —CO₂N(R⁵)₂; ═CH₂, ═CHR″ or ═C(R″)₂;
wherein at least one of R² and R³ are ═CH₂, ═CHR″ or ═C(R¹¹)₂;
R⁴ is hydrogen, halogen or C1-C3 alkyl;
each R⁵ is independently hydrogen, cyclopropyl or C1-C3 alkyl;
each R⁶ is independently hydrogen, hydroxy, C1-C4 hydroxyalkyl or heteroaryl, or two R⁶ join to form C3-C6 cycloalkyl or heterocycle;
each R⁷ is independently hydrogen, C1-C3 alkyl, hydroxy, halogen, halo-C1-C3 alkyl, di-halo-C1-C3 alkyl, tri-halo-C1-C3 alkyl, —NH₂, —NH(C1-C3 alkyl), —N(C1-C3 alkyl)₂, oxo (═O), —O—(C1-C3 alkyl), —(C1-C3 alkyl)-OH, —C(O)OH, —C(O)O(C1-C3 alkyl), —O—CH₂—C(O)NH₂, L—C(O)NH₂, —C(O)NH(C1-C3 alkyl), —NHC(O)(C1-C3 alkyl), —C(O)N(C1-C3 alkyl)₂, —CN, aryl, dialkylphosphine oxide, —S(O)₂NH(CH₃), sulfone, L-heterocycle optionally substituted with 1-2 substituents selected from L-hydroxy, oxo (═O), C1-C3 alkyl and C3 cycloalkyl, or L-heteroaryl optionally substituted with 1-2 substituents selected from L-hydroxy, —NH₂, C1-C3 alkyl, C1-C3 haloalkyl, C3 cycloalkyl, —C(O)NH(C3-C4 cycloalkyl) and —NHC(O)(C1-C3 alkyl),
two R⁷ on the same atom optionally join to form a spirocyclic ring selected from C3-C6 cycloalkyl and heterocycle, where said spirocyclic ring is optionally substituted with 1-2 substituents selected from oxo (═O), halogen, hydroxy, C1-C3 alkyl and —O—(C1-C3 alkyl),
two R⁷ on adjacent atoms optionally join to form a bond or a fused ring selected from C3-C6 cycloalkyl optionally substituted with 1-4 R⁸, heteroaryl optionally substituted with 1-4R⁸, aryl optionally substituted with 1-4R⁸, and heterocycle optionally substituted with 1-4R⁸, and
two R⁸ on non-adjacent atoms optionally join to form a 1-2 carbon bridge;
each R⁸ is independently C1-C3 alkyl, L-hydroxy, halogen, halo-C1-C3 alkyl, di-halo-C1-C3 alkyl, tri-halo-C1-C3 alkyl, —N(R⁵)₂, oxo (═O), —O—(C1-C3 alkyl), —(C1-C3 alkyl)-OH, —C(O)OR⁵, —C(O)B′, —C(OR⁵)(R⁵)₂,-(C1-C3 alkyl)C(O)N(R⁵)₂, —C(O)N(R⁵)₂, —C(O)N(R¹⁰)₂, —CN, L-L-heteroaryl or L-heterocycle each optionally substituted with C1-C3 alkyl, C1-C3 haloalkyl, —CH₂—S—CH₃, —S(O)₂NH₂ or —S(O)₂(C1-C3 alkyl);
each R⁹ is independently C1-C3 alkyl, hydroxy, halogen, oxo (═O), —O—(C1-C3 alkyl), —(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂, L-heteroaryl or —CN, or
two R⁹ join to form a bond or —S(O)(CH₃)₂;
each R¹⁰ is independently hydrogen, C1-C3 alkyl, halogen, or joins with R⁷ or another R¹⁰ to form a heterocyclic ring;
each R¹¹ is independently halogen;
each L is independently a bond;-C1-C4 alkylene-, —NR⁵—, or —C(O)—;
each n is 0-3;
o is 1-6;
p is 1-8; and
q is 0-1.

In another aspect of the invention, pharmaceutical compositions are provided comprising a therapeutically effective amount of a compound of the present invention or a pharmaceutically acceptable salt thereof and a pharmaceutically acceptable excipient.

In yet another aspect of the invention, methods for inhibiting the activity of cells containing wild type KRas or one or more KRas mutations, for instance the KRas mutations G12A, G12C, G12D, G12R, G12S, G12V, G13D or Q61H, in a in a cell, comprising contacting the cell with a compound of Formula (I), or a pharmaceutically acceptable salt thereof, or a pharmaceutical composition thereof as defined herein. In one embodiment, the contacting is in vitro. In one embodiment, the contacting is in vivo.

Also provided herein is a method of inhibiting cell proliferation, in vitro or in vivo, the method comprising contacting a cell with an effective amount of a compound of Formula (I), or a pharmaceutically acceptable salt thereof, or a pharmaceutical composition thereof as defined herein.

Also provided are methods for treating cancer in a patient comprising administering a therapeutically effective amount of a compound or pharmaceutical composition of the present invention or a pharmaceutically acceptable salt thereof to a patient in need thereof.

Also provided herein is a method of treating a KRas wild type-associated or KRas G12A, G12C, G12D, G12R, G12S, G12V, G13D and/or Q61H-associated disease or disorder in a patient in need of such treatment, the method comprising administering to the patient a therapeutically effective amount of a compound of Formula (I), or a pharmaceutically acceptable salt thereof, or a pharmaceutical composition thereof as defined herein.

Also provided herein is a compound of Formula (I), or a pharmaceutically acceptable salt thereof, or a pharmaceutical composition thereof as defined herein for use in therapy.

Also provided herein is a compound of Formula (I), or a pharmaceutically acceptable salt thereof or a pharmaceutical composition thereof as defined herein for use in the treatment of cancer.

Also provided herein is a compound of Formula (I), or a pharmaceutically acceptable salt thereof for use in the inhibition of wild type KRas or multiple types of KRas mutations, for instance KRas G12A, G12C, G12D, G12R, G12S, G12V, G13D and/or Q61H mutations.

Also provided herein is a compound of Formula (I), or a pharmaceutically acceptable salt thereof or a pharmaceutical composition thereof as defined herein, for use in the treatment of wild type KRas or a KRas mutation G12A, G12C, G12D, G12R, G12S, G12V, G13D and/or Q61H-associated disease or disorder.

Also provided herein is the use of a compound of Formula (I), or a pharmaceutically acceptable salt thereof, as defined herein in the manufacture of a medicament for the treatment of cancer.

Also provided herein is a use of a compound of Formula (I), or a pharmaceutically acceptable salt thereof, as defined herein in the manufacture of a medicament for the inhibition of activity of wild type KRas or mutated forms of KRas, including the mutations: G12A, G12C, G12D, G12R, G12S, G12V, G13D and/or Q61H.

Also provided herein is the use of a compound of Formula (I), or a pharmaceutically acceptable salt thereof, as defined herein, in the manufacture of a medicament for the treatment of a wild type KRas-associated or a KRas G12A, G12C, G12D, G12R, G12S, G12V, G13D and/or Q61H-associated disease or disorder.

Also provided herein is a method for treating cancer in a patient in need thereof, the method comprising (a) determining that the cancer is associated with wild type KRas or a KRas G12A, G12C, G12D, G12R, G12S, G12V, G13D and/or Q61H mutation (i.e., a wild type KRas-associated or a KRas G12A, G12C, G12D, G12R, G12S, G12V, G13D and/or Q61HG12X-associated cancer); and (b) administering to the patient a therapeutically effective amount of a compound of Formula (I), or a pharmaceutically acceptable salt thereof, or a pharmaceutical composition thereof.

One potential utility of the herein-described pan-KRas inhibitors, including pan-KRas inhibitors such as (R)-1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-methylpiperidin-3-ol (Example 5 in 63/125,776), is for the treatment of cancers that develop resistance following long-term treatment with KRas G12C inhibitors. Thus, embodiments of the invention include those wherein a patient suffering from cancer is treated with a herein-described pan-KRas inhibitor after treatment with a G12C inhibitor becomes ineffective or less effective due to the emergence of resistance-imparting mutations.

Treatment of KRas G12C mutant cancers with covalent KRas G12C inhibitors such as adagrasib (MRTX849) or sotorasib (AMG510) may result in the incorporation of additional mutations that confer resistance to adagrasib. These mutations could confer resistance through numerous mechanisms.

Mutations that change the mutant cysteine at codon 12 to another amino acid would render the current covalent KRas G12C inhibitors ineffective since current inhibitors make a covalent bond with the mutant cysteine amino acid side chain. Likewise, in patients that have one wild type KRas allele in addition to the KRas G12C-mutant allele, mutations in the wild type codon 12 glycine to another codon would allow bypass signaling in these tumors through the novel mutant protein. The repertoire of codon 12 mutations that can occur with a single nucleotide substitution in the wild type gene (glycine codon) includes mutations commonly observed in cancer such as G12S, G12V, G12R, G12C. The repertoire of codon 12 mutations that can occur with single nucleotide base substitutions of the cysteine codon 12 include mutations not frequently observed in cancer, G12Y, G12F and G12W, in addition to G12S and G12R.

Second-site mutations may also occur in another location in the KRas G12C mutant gene that confers resistance to KRas G12C inhibitor treatment. These mutations may confer resistance through different mechanisms. RAS proteins are small GTPases that normally cycle between an active, GTP-bound state and an inactive, GDP-bound state. RAS proteins are loaded with GTP through guanine nucleotide exchange factors (GEFs; e.g., SOS1) which are activated by upstream receptor tyrosine kinases, triggering subsequent interaction with effector proteins that activate RAS-dependent signaling. RAS proteins hydrolyze GTP to GDP through their intrinsic GTPase activity which is dramatically enhanced by GTPase-activating proteins (GAPs). Mutations at codons 12 and 13 in RAS proteins impair GAP-stimulated GTP hydrolysis leaving RAS predominantly in the GTP-bound, active state. Covalent KRas G12C inhibitors in current clinical development only bind GDP-bound KRas G12C. Mutations such as Q61 codon mutations, which may or may not occur on the same allele as the G12C mutation, reduce the intrinsic GTPase activity of KRas and may represent a mechanism of resistance to KRas G12C inhibitor treatment by shifting KRas into the GTP-loaded state where it is not susceptible to covalent inhibition. Co-mutations such as R68, H95 and Y96 may be present along with the KRas G12C mutation and may diminish the binding affinity of KRas G12C inhibitors to the Switch II binding pocket.

The herein-described pan-KRas inhibitors may demonstrate activity against common as well as uncommon codon 12 mutations or mutations that occur in the KRas protein that diminish binding of KRas G12C inhibitors to the KRas protein.

Also provided herein is a process for preparing a compound of Formula (I), or a pharmaceutically acceptable salt thereof.

Also provided herein is a compound of Formula (I), or a pharmaceutically acceptable salt thereof obtained by a process of preparing the compound as defined herein.

### DETAILED DESCRIPTION OF THE INVENTION

The present invention relates to inhibitors of wild type KRas or multiple mutated forms of KRas, for instance KRas G12A, G12C, G12D, G12R, G12S, G12V, G13D and/or Q61H mutations. In particular, the present invention relates to compounds that inhibit the activity of wild type KRas or KRas mutations such as G12A, G12C, G12D, G12R, G12S, G12V, G13D and/or Q61H, pharmaceutical compositions comprising a therapeutically effective amount of the compounds and methods of use therefor.

### Definitions

Unless defined otherwise, all technical and scientific terms used herein have the same meaning as is commonly understood by one of skill in the art to which this invention belongs. All patents, patent applications, and publications referred to herein are incorporated by reference.

As used herein, “KRas G12A” refers to a mutant form of a mammalian KRas protein that contains an amino acid substitution of an alanine for a glycine at amino acid position 12. The assignment of amino acid codon and residue positions for human KRas is based on the amino acid sequence identified by UniProtKB/Swiss-Prot P01116: Variantp.Glyl2Asp.s used herein, a “KRas G12A inhibitor” refers to compounds of the present invention that are represented by Formula (I), as described herein. These compounds are capable of negatively modulating or inhibiting all or a portion of the enzymatic activity of KRas G12A. A “KRas G12A-associated disease or disorder” as used herein refers to diseases or disorders associated with or mediated by or having a KRas G12A mutation. A non-limiting example of a KRas G12A-associated disease or disorder is a KRas G12A-associated cancer.

As used herein, “KRas G12C” refers to a mutant form of a mammalian KRas protein that contains an amino acid substitution of a cysteine for a glycine at amino acid position 12. The assignment of amino acid codon and residue positions for human KRas is based on the amino acid sequence identified by UniProtKB/Swiss-Prot P01116: Variantp.Gly12Asp.s used herein, a “KRas G12C inhibitor” refers to compounds of the present invention that are represented by Formula (I), as described herein. These compounds are capable of negatively modulating or inhibiting all or a portion of the enzymatic activity of KRas G12C. A “KRas G12C-associated disease or disorder” as used herein refers to diseases or disorders associated with or mediated by or having a KRas G12C mutation. A non-limiting example of a KRas G12C-associated disease or disorder is a KRas G12CD-associated cancer.

As used herein, “KRas G12D” refers to a mutant form of a mammalian KRas protein that contains an amino acid substitution of an aspartic acid for a glycine at amino acid position 12. The assignment of amino acid codon and residue positions for human KRas is based on the amino acid sequence identified by UniProtKB/Swiss-Prot P01116: Variantp.Gly12Asp.s used herein, a “KRas G12D inhibitor” refers to compounds of the present invention that are represented by Formula (I), as described herein. These compounds are capable of negatively modulating or inhibiting all or a portion of the enzymatic activity of KRas G12D. A “KRas G12D-associated disease or disorder” as used herein refers to diseases or disorders associated with or mediated by or having a KRas G12D mutation. A non-limiting example of a KRas G12D-associated disease or disorder is a KRas G12D-associated cancer.

As used herein, “KRas G12R” refers to a mutant form of a mammalian KRas protein that contains an amino acid substitution of an arginine for a glycine at amino acid position 12. The assignment of amino acid codon and residue positions for human KRas is based on the amino acid sequence identified by UniProtKB/Swiss-Prot P01116: Variantp.Glyl2Asp.s used herein, a “KRas G12R inhibitor” refers to compounds of the present invention that are represented by Formula (I), as described herein. These compounds are capable of negatively modulating or inhibiting all or a portion of the enzymatic activity of KRas G12R. A “KRas G12R-associated disease or disorder” as used herein refers to diseases or disorders associated with or mediated by or having a KRas G12R mutation. A non-limiting example of a KRas G12R-associated disease or disorder is a KRas G12R-associated cancer.

As used herein, “KRas G12S” refers to a mutant form of a mammalian KRas protein that contains an amino acid substitution of a serine for a glycine at amino acid position 12. The assignment of amino acid codon and residue positions for human KRas is based on the amino acid sequence identified by UniProtKB/Swiss-Prot P01116: Variantp.Gly12Asp.s used herein, a “KRas G12S inhibitor” refers to compounds of the present invention that are represented by Formula (I), as described herein. These compounds are capable of negatively modulating or inhibiting all or a portion of the enzymatic activity of KRas G12S. A “KRas G12S-associated disease or disorder” as used herein refers to diseases or disorders associated with or mediated by or having a KRas G12S mutation. A non-limiting example of a KRas G12S-associated disease or disorder is a KRas G12S-associated cancer.

As used herein, “KRas G12V” refers to a mutant form of a mammalian KRas protein that contains an amino acid substitution of a valine for a glycine at amino acid position 12. The assignment of amino acid codon and residue positions for human KRas is based on the amino acid sequence identified by UniProtKB/Swiss-Prot P01116: Variantp.Gly12Asp.s used herein, a “KRas G12V inhibitor” refers to compounds of the present invention that are represented by Formula (I), as described herein. These compounds are capable of negatively modulating or inhibiting all or a portion of the enzymatic activity of KRas G12V. A “KRas G12V-associated disease or disorder” as used herein refers to diseases or disorders associated with or mediated by or having a KRas G12V mutation. A non-limiting example of a KRas G12V-associated disease or disorder is a KRas G12V-associated cancer.

As used herein, “KRas G13D” refers to a mutant form of a mammalian KRas protein that contains an amino acid substitution of an aspartic acid for a glycine at amino acid position 13. The assignment of amino acid codon and residue positions for human KRas is based on the amino acid sequence identified by UniProtKB/Swiss-Prot P01116: Variantp.Glyl2Asp.s used herein, a “KRas G13D inhibitor” refers to compounds of the present invention that are represented by Formula (I), as described herein. These compounds are capable of negatively modulating or inhibiting all or a portion of the enzymatic activity of KRas G13D. A “KRas G13D-associated disease or disorder” as used herein refers to diseases or disorders associated with or mediated by or having a KRas G13D mutation. A non-limiting example of a KRas G13D-associated disease or disorder is a KRas G13D-associated cancer.

As used herein, “KRas Q61H” refers to a mutant form of a mammalian KRas protein that contains an amino acid substitution of a histidine for a glutamine at amino acid position 61. The assignment of amino acid codon and residue positions for human KRas is based on the amino acid sequence identified by UniProtKB/Swiss-Prot P01116. Variantp.Gly12Asp.s used herein, a “KRas Q61H inhibitor” refers to compounds of the present invention that are represented by Formula (I), as described herein. These compounds are capable of negatively modulating or inhibiting all or a portion of the enzymatic activity of KRas Q61H. A “KRas Q61H-associated disease or disorder” as used herein refers to diseases or disorders associated with or mediated by or having a KRas Q61H mutation. A non-limiting example of a KRas Q61H-associated disease or disorder is a KRas Q61H-associated cancer.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having wild type KRas or a KRas G12A, G12C, G12D, G12R, G12S, G12V, G13D and/or Q61H mutation (e.g., as determined using a regulatory agency-approved, e.g., FDA-approved, assay or kit). In some embodiments, the subject has a tumor that is positive for wild type KRas or a KRas G12A, G12C, G12D, G12R, G12S, G12V, G13D and/or Q61H mutation (e.g., as determined using a regulatory agency-approved assay or kit). The subject can be a subject with a tumor(s) that is positive for wild type KRas or a KRas G12A, G12C, G12D, G12R, G12S, G12V, G13D and/or Q61H mutation (e.g., identified as positive using a regulatory agency-approved, e.g., FDA-approved, assay or kit). The subject can be a subject whose tumors have wild type KRas or a KRas G12A, G12C, G12D, G12R, G12S, G12V, G13D and/or Q61H mutation (e.g., where the tumor is identified as such using a regulatory agency-approved, e.g., FDA-approved, kit or assay). In some embodiments, the subject is suspected of having wild type KRas-associated or a KRas G12A, KRas G12C, KRas G12D, KRas G12R, KRas G12S, KRas G12V, KRas G13D or KRas Q61H gene-associated cancer. In some embodiments, the subject has a clinical record indicating that the subject has a tumor that has wild type KRas or a KRas G12A, G12C, G12D, G12R, G12S, G12V, G13D and/or Q61H mutation (and optionally the clinical record indicates that the subject should be treated with any of the compositions provided herein).

In some embodiments of any of the methods or uses described herein, an assay is used to determine whether the patient has wild type KRas or a KRas G12A, G12C, G12D, G12R, G12S, G12V, G13D and/or Q61H mutation using a sample (e.g., a biological sample or a biopsy sample (e.g., a paraffin-embedded biopsy sample) from a patient (e.g., a patient suspected of having wild type KRas or a KRas G12A, G12C, G12D, G12R, G12S, G12V, G13D and/or Q61H-associated cancer, a patient having one or more symptoms of a wild type KRas-associated or a KRas G12A, G12C, G12D, G12R, G12S, G12V, G13D and/or Q61H-associated cancer, and/or a patient that has an increased risk of developing a wild type KRas-associated or a KRas G12A, G12C, G12D, G12R, G12S, G12V, G13D and/or Q61H-associated cancer) can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof.

The term “regulatory agency” is a country's agency for the approval of the medical use of pharmaceutical agents with the country. For example, a non-limiting example of a regulatory agency is the U.S. Food and Drug Administration (FDA).

The term “acyl” refers to —C(O)CH₃.

The terms “C1-C6 alkyl”, “C1-C4 alkyl” and “C1-C3 alkyl” as employed herein refers to straight and branched chain aliphatic groups having from 1-6 carbon atoms, or 1-4 carbon atoms, or 1-3 carbon atoms, respectively. Examples of alkyl groups include, without limitation, methyl, ethyl, propyl, isopropyl, butyl, isobutyl, sec-butyl, tert-butyl, pentyl, and hexyl.

The terms “C1-C3 haloalkyl” and “C1-C4 haloalkyl” refer to a C1-C3 alkyl chain or C1-C4 alkyl chain, respectively, as defined herein in which one or more hydrogen has been replaced by a halogen. Examples include trifluoromethyl, difluoromethyl and fluoromethyl.

An “C1-C4 alkylene,” group is a C1-C4 alkyl group, as defined hereinabove, that is positioned between and serves to connect two other chemical groups. Exemplary alkylene groups include, without limitation, methylene, ethylene, propylene, and butylene.

The terms “C1-C3 alkoxy” and “C1-C4 alkoxy” refer to -OCI -C3 alkyl and -OC1-C4 alkyl, respectively, wherein the alkyl portion is as defined herein above.

The term “cycloalkyl” as employed herein includes saturated and partially unsaturated cyclic hydrocarbon groups having 3 to 12 carbons, for example 3 to 8 carbons, and as a further example 3 to 6 carbons, wherein the cycloalkyl group additionally is optionally substituted with one or more R⁸ or R⁹ groups as defined herein. Examples of cycloalkyl groups include, without limitation, cyclopropyl, cyclobutyl, cyclopentyl, cyclopentenyl, cyclohexyl, cyclohexenyl, cycloheptyl, and cyclooctyl. The term “cycloalkyl” also includes bridged cycloalkyls, such as bicyclo[1.1.1]pentanyl.

As used herein, the terms “C1-C3 hydroxyalkyl” and “C1-C4 hydroxyalkyl” refer to -C1-C3 alkylene-OH and -C1-C4 alkylene-OH, respectively.

As used herein, the term “C2-C4 hydroxyalkynyl” refers to -C2-C4 alkynylene-OH.

An “aryl” group is a C6-C14 aromatic moiety comprising one to three aromatic rings, which is optionally substituted with one or more substituents as defined herein and in Formula I. As one embodiment, the aryl group is a C6-C10 aryl group. Examples of aryl groups include, without limitation, phenyl, naphthyl, anthracenyl, fluorenyl, and dihydrobenzofuranyl. “Aryl” also refers to bicyclic or tricyclic ring systems in which one or two rings, respectively, of said aryl ring system may be saturated or partially saturated, and wherein if said ring system includes two saturated rings, said saturated rings may be fused or spirocyclic. An example of an aryl ring system comprising two saturated rings wherein the rings are spirocyclic includes the

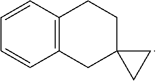 [SMILES: c1ccc2c(c1)CCC1(CC1)C2]

following ring system:

An “araC1-C6 alkyl” or “arylalkyl” group comprises an aryl group covalently linked to an alkyl group, either of which may independently be optionally substituted or unsubstituted. An example of an aralkyl group is (C6-C10)aryl(C1-C6)alkyl-, including, without limitation, benzyl, phenethyl, and naphthylmethyl. An example of a substituted araC1-C6 alkyl is wherein the alkyl group is substituted with hydroxyalkyl.

A “heterocyclyl” or “heterocyclic” group is a saturated or partially unsaturated ring structure having from 3 to 12 atoms, for example 4 to 8 atoms, wherein one or more atoms are selected from the group consisting of N, O, and S wherein the ring N atom may be oxidized to N-0, and the ring S atom may be oxidized to SO or S02, the remainder of the ring atoms being carbon. The heterocyclyl may be a monocyclic, a bicyclic, a spirocyclic or a bridged ring system. The heterocyclic group is optionally substituted on ring carbon or ring nitrogen at one or more positions as defined herein and in Formula I. The heterocyclic group is also independently optionally substituted on a ring nitrogen atom with alkyl, aralkyl, alkylcarbonyl, or on sulfur with lower alkyl. Examples of heterocyclic groups include, without limitation, epoxy, azetidinyl, aziridinyl, tetrahydrofuranyl, tetrahydropyranyl, pyrrolidinyl, pyrrolidinonyl, piperidinyl, piperazinyl, imidazolidinyl, imidazopyridinyl, thiazolidinyl, dithianyl, trithianyl, dioxolanyl, oxazolidinyl, oxazolidinonyl, decahydroquinolinyl, piperidonyl, 4-piperidinonyl, quinuclidinyl, thiomorpholinyl, thiomorpholinyl 1,1 dioxide, morpholinyl, azepanyl, oxazepanyl, azabicyclohexanyls, azabicycloheptanyl, azabicyclooctanyls, azabicyclononanyls (e.g., octahydroindolizinyl), azaspiroheptanyls, dihydro-1H,3H,5H-oxazolo[3,4-c]oxazolyl, tetrahydro-1′H,3′H-spiro[cyclopropane-1,2′-pyrrolizine], hexahydro-1H-pyrrolizinyl, hexahydro-1H-pyrrolo[2,1-c][1,4]oxazinyl, octahydroindolizinyl, oxaazaspirononanyls, oxaazaspirooctanyls, diazaspirononanyls, oxaazabiocycloheptanyls, hexahydropyrrolizinyl 4(1H)-oxide, tetrahydro-2H-thiopyranyl 1-oxide and tetrahydro-2H-thiopyranyl 1,1-dioxide. Specifically excluded from the scope of this term are compounds having adjacent annular 0 and/or S atoms.

As used herein, the term “heteroaryl” refers to groups having 5 to 14 ring atoms, preferably 5, 6, 9, or 10 ring atoms; having 6, 10, or 14 7t electrons shared in a cyclic array; and having, in addition to carbon atoms, from one to three heteroatoms per ring, or from one to three heteroatoms in at least one ring, selected from the group consisting of N, O, and S. Examples of heteroaryl groups include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, 6,7-dihydro-5H-pyrrolo[1,2-a]imidazole, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. “Heteroaryl” also refers to bicyclic ring systems having, in addition to carbon atoms, from one to three heteroatoms per ring selected from the group consisting of N, O, and S in which one ring system may be saturated or partially saturated.

As used herein, “an effective amount” of a compound is an amount that is sufficient to negatively modulate or inhibit the activity of one or more of wild type KRas or a KRas G12A, KRas G12C, KRas G12D, KRas G12R, KRas G12S, KRas G12V, KRas G13D or KRas Q61H. Such amount may be administered as a single dosage or may be administered according to a regimen, whereby it is effective.

As used herein, a “therapeutically effective amount” of a compound is an amount that is sufficient to ameliorate, or in some manner reduce a symptom or stop or reverse progression of a condition, or negatively modulate or inhibit the activity of wild type KRas or one or more of KRas G12A, KRas G12C, KRas G12D, KRas G12R, KRas G12S, KRas G12V, KRas G13D or KRas Q61H. Such amount may be administered as a single dosage or may be administered according to a regimen, whereby it is effective.

As used herein, treatment means any manner in which the symptoms or pathology of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

### Compounds

Certain embodiments of the invention include compounds of Formula (I):

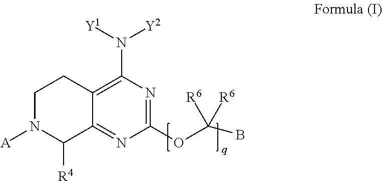

or a pharmaceutically acceptable salt thereof, wherein:
A is aryl or heteroaryl, wherein the aryl or the heteroaryl is optionally substituted with 1-4 R¹;
B is selected from:

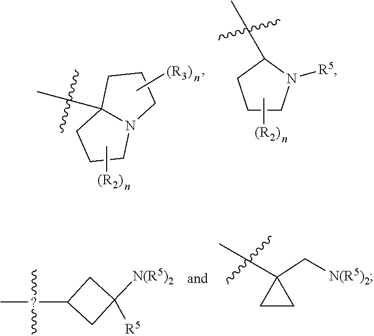

Y¹ is L-hydrogen optionally substituted with 1-4 R⁸, hydroxy, halogen, L—C3-C6 cycloalkyl optionally substituted with 1-4 R⁹, L—S(O)₂N(R⁵)₂ optionally substituted with 1-4 R⁹, L-heteroaryl optionally substituted with 1-4 R⁸, L-aryl optionally substituted with 1-4 R⁸, and L-heterocycle substituted with 1-2 oxo (═O) or oxo-containing substituent and optionally further substituted with 1-2 heteroaryl-R⁸ or R⁸.
Y² is hydrogen or C1-C4 alkyl;
or Y¹ and Y² join to form:

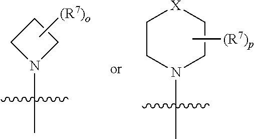

where X is selected from: a bond, —S—, —O—, —N<bound to a fused ring, —CH₂—, —CH₂—NH—, —CH₂—NH—CH₂—, —CH₂—CH₂—CH₂—, —CH₂—CH₂—, —O—CH₂— and —S—CH₂—;

each R¹ is independently halogen, cyano, hydroxy, C1-C4 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, —S—C1-C3 haloalkyl, C1-C3 alkoxy, hydroxyC1-C3 alkyl, —CH₂C(═O)N(R⁵)₂, —C3-C4 alkynyl(NR⁵)₂, —N(R⁵)₂, deuteroC2-C4 alkynyl, (C1-C3 alkoxy)haloC1-C3 alkyl-, or C3-C6 cycloalkyl wherein said C3-C6 cycloalkyl is optionally substituted with halogen or C1-C3 alkyl;
each R² is independently hydrogen, hydroxy, halogen, C1-C3 alkyl, C1-C3 cyanoalkyl, C1-C3 hydroxyalkyl, HC(═O)—, —OC(O)N(R⁵)₂, —CH₂OC(O)N(R⁵)₂, —CH₂NR⁵—SO₂—N(R⁵)₂, —CO₂R⁵, —CO₂N(R⁵)₂, ═CH₂, ═CHR″ or ═C(R″)₂;
each R³ is independently hydrogen, hydroxy, halogen, L—C1-C3 alkyl, C1-C3 cyanoalkyl, C1-C3 hydroxyalkyl, HC(═O)—, —OC(O)N(R⁵)₂, —CH₂OC(O)N(R5)₂, —CH₂NR⁵—SO₂—N(R⁵)₂, —CO₂R⁵, —CO₂N(R⁵)₂; ═CH₂, ═CHR″ or ═C(R″)₂;
wherein at least one of R² and R³ are ═CH₂, ═CHR″ or ═C(R¹¹)₂;
R⁴ is hydrogen, halogen or C1-C3 alkyl;
each R⁵ is independently hydrogen, cyclopropyl or C1-C3 alkyl;
each R⁶ is independently hydrogen, hydroxy, C1-C4 hydroxyalkyl or heteroaryl, or two R⁶ join to form C3-C6 cycloalkyl or heterocycle;
each R⁷ is independently hydrogen, C1-C3 alkyl, hydroxy, halogen, halo-C1-C3 alkyl, di-halo-C1-C3 alkyl, tri-halo-C1-C3 alkyl, —NH₂, —NH(C1-C3 alkyl), —N(C1-C3 alkyl)₂, oxo (═O), —O—(C1-C3 alkyl), —(C1-C3 alkyl)-OH, —C(O)OH, —C(O)O(C1-C3 alkyl), —O—CH₂—C(O)NH₂, L—C(O)NH₂, —C(O)NH(C1-C3 alkyl), —NHC(O)(C1-C3 alkyl), —C(O)N(C1-C3 alkyl)₂, —CN, aryl, dialkylphosphine oxide, —S(O)₂NH(CH₃), sulfone, L-heterocycle optionally substituted with 1-2 substituents selected from L-hydroxy, oxo (═O), C1-C3 alkyl and C3 cycloalkyl, or L-heteroaryl optionally substituted with 1-2 substituents selected from L-hydroxy, —NH₂, C1-C3 alkyl, C1-C3 haloalkyl, C3 cycloalkyl, —C(O)NH(C3-C4 cycloalkyl) and —NHC(O)(C1-C3 alkyl),
two R⁷ on the same atom optionally join to form a spirocyclic ring selected from C3-C6 cycloalkyl and heterocycle, where said spirocyclic ring is optionally substituted with 1-2 substituents selected from oxo (═O), halogen, hydroxy, C1-C3 alkyl and —O—(C1-C3 alkyl),
two R⁷ on adjacent atoms optionally join to form a bond or a fused ring selected from C3-C6 cycloalkyl optionally substituted with 1-4 R⁸, heteroaryl optionally substituted with 1-4R⁸, aryl optionally substituted with 1-4R⁸, and heterocycle optionally substituted with 1-4R⁸, and
two R⁸ on non-adjacent atoms optionally join to form a 1-2 carbon bridge;
each R⁸ is independently C1-C3 alkyl, L-hydroxy, halogen, halo-C1-C3 alkyl, di-halo-C1-C3 alkyl, tri-halo-C1-C3 alkyl, —N(R⁵)₂, oxo (═O), —O—(C1-C3 alkyl), —(C1-C3 alkyl)-OH, —C(O)OR⁵, —C(O)B′, —C(OR⁵)(R⁵)₂,-(C1-C3 alkyl)C(O)N(R⁵)₂, —C(O)N(R⁵)₂, —C(O)N(R¹⁰)₂, —CN, L-L-heteroaryl or L-heterocycle each optionally substituted with C1-C3 alkyl, C1-C3 haloalkyl, —CH₂—S—CH₃, —S(O)₂NH₂ or —S(O)₂(C1-C3 alkyl);
each R⁹ is independently C1-C3 alkyl, hydroxy, halogen, oxo (═O), —O—(C1-C3 alkyl), —(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂, L-heteroaryl or —CN, or
two R⁹ join to form a bond or —S(O)(CH₃)₂;
each R^{1D} is independently hydrogen, C1-C3 alkyl, halogen, or joins with R⁷ or another R¹⁰ to form a heterocyclic ring;
each R¹¹ is independently halogen;
each L is independently a bond;-C1-C4 alkylene-, —NR⁵—, or —C(O)—;
each n is 0-3;
o is 1-6;
p is 1-8; and
q is 0-1.
Certain embodiments of the invention include such compounds or salts wherein:
q is 1;
A is naphthyl; and
B is:

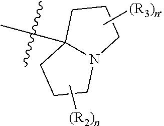

Certain embodiments of the invention include such compounds or salts wherein:
q is I;
A is naphthyl; and
B is:

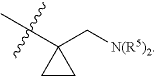

Certain embodiments of the invention include such compounds or salts wherein Y¹ is hydrogen, hydroxy, halogen or L-heteroaryl optionally substituted with 1-4R⁸, and Y² is hydrogen or C1-C4 alkyl.

Certain embodiments of the invention include such compounds or salts wherein Y¹ and Y² join to form:

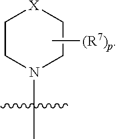

Certain embodiments of the invention include such compounds or salts wherein X is —CH₂—NH—, and two R⁷ join to form a fused heteroaryl ring substituted with 1-4 R⁸ where one R⁸ is —C(O)N(R¹⁰)₂.

Certain embodiments of the invention include such compounds or salts wherein the fused heteroaryl ring is pyrazolyl, one R⁸ is —C(O)N(R¹⁰)₂ and one R⁸ is halogen or C1-C3 alkyl.

Certain embodiments of the invention include such compounds or salts wherein X is a bond, and two R⁷ join to form a fused heterocyclyl ring, optionally substituted with one or two oxo.

Certain embodiments of the invention include such compounds or salts wherein X is —CH₂—, and two R⁷ join to form a spirocyclic heterocyclyl ring substituted with one or two oxo.

Certain embodiments of the invention include such compounds or salts wherein at least one R¹ is C1-C4 alkyl.

Certain embodiments of the invention include such compounds or salts wherein at least one R¹ is halogen.

Certain embodiments of the invention include such compounds or salts wherein said halogen is a fluorine.

Certain embodiments of the invention include such compounds or salts wherein at least one R¹ is hydroxy.

Certain embodiments of the invention include such compounds or salts wherein one R² is C1-C4 alkyl.

Certain embodiments of the invention include such compounds or salts wherein at least one R² is halogen.

Certain embodiments of the invention include such compounds or salts wherein said halogen is a fluorine.

Certain embodiments of the invention include such compounds or salts wherein at least one R² is hydroxy.

Certain embodiments of the invention include such compounds or salts wherein at least one R3 is C1-C4 alkyl.

Certain embodiments of the invention include such compounds or salts wherein at least one R³ is halogen.

Certain embodiments of the invention include such compounds or salts wherein said halogen is fluorine.

Certain embodiments of the invention include such compounds or salts wherein at least one R² is ═CH₂, ═CHR″ or ═C(R″)₂.

Certain embodiments of the invention include such compounds or salts wherein R¹¹ is F.

Certain embodiments of the invention include such compounds or salts wherein at least one R³ is hydroxy.

Certain embodiments of the invention include such compounds or salts wherein at least one R3 is ═CH₂, ═CHR″ or ═C(R″)₂.

Certain embodiments of the invention include such compounds or salts wherein R¹¹ is F.

Certain embodiments of the invention include such compounds or salts wherein R⁴ is halogen.

Certain embodiments of the invention include such compounds or salts wherein said halogen is fluorine.

Certain embodiments of the invention include such compounds or salts wherein at least one R⁵ is C1-C4 alkyl.

Certain embodiments of the invention include such compounds or salts wherein at least one R⁵ is hydrogen.

Certain embodiments of the invention include such compounds or salts wherein one or both R⁶ are hydrogen or C1-C4 alkyl.

Certain embodiments of the invention include such compounds or salts wherein two R⁶ join to form C3-C6 cycloalkyl or heterocycle.

Certain embodiments of the invention include such compounds or salts wherein Y¹ is L-C3-C6 cycloalkyl, L-heteroaryl, L-aryl, or L-heterocycle, where L is a bond, C1-C4 alkyl, NH or N(C1-C3) alkyl.

Certain embodiments of the invention include such compounds or salts wherein Y¹ is L-heteroaryl.

Certain embodiments of the invention include such compounds or salts wherein the heteroaryl is thietane dioxide, iso-thiazolidine dioxide, imidazopyrazine, pyridine or pyrimidine.

Certain embodiments of the invention include such compounds or salts wherein Y¹ is L-C3-C6 cycloalkyl.

Certain embodiments of the invention include such compounds or salts wherein the cycloalkyl is cyclobutane, cyclopentane, cyclohexane or cycloheptane.

Certain embodiments of the invention include such compounds or salts wherein Y¹ is L-heterocycle.

Certain embodiments of the invention include such compounds or salts wherein the heterocycle is pyrrolidinone.

Certain embodiments of the invention include such compounds or salts wherein Y² is hydrogen.

Certain embodiments of the invention include such compounds or salts wherein Y² is C1-C4 alkyl;

Certain embodiments of the invention include such compounds or salts wherein at least one R⁸ is C1-C4 alkyl.

Certain embodiments of the invention include such compounds or salts wherein at least one R⁸ is hydroxy or C1-C3 alkyl-hydroxy.

Certain embodiments of the invention include such compounds or salts wherein one or two R⁸ are oxo (═O).

Certain embodiments of the invention include such compounds or salts wherein at least one R⁸ is aryl or heteroaryl.

Certain embodiments of the invention include such compounds or salts wherein at least one R⁸ is C(O)OH.

Certain embodiments of the invention include such compounds or salts wherein at least one R⁸ is —C(O)NH₂, —C(O)NH(C1-C3 alkyl) or —C(O)N(C1-C3 alkyl)₂.

Certain embodiments of the invention include such compounds or salts wherein at least one R⁸ is —NH₂, —NH(C1-C3 alkyl); —N(C1-C3 alkyl)₂.

Certain embodiments of the invention include such compounds or salts wherein at least one R9 is C1-C4 alkyl.

Certain embodiments of the invention include such compounds or salts wherein at least one R9 is hydroxy or C1-C3 alkyl-hydroxy.

Certain embodiments of the invention include such compounds or salts wherein one or two R⁹ is oxo (═O).

Certain embodiments of the invention include such compounds or salts wherein at least one R9 is aryl or heteroaryl.

Certain embodiments of the invention include such compounds or salts wherein at least one R9 is C(O)OH.

Certain embodiments of the invention include such compounds or salts wherein at least one R9 is —C(O)NH₂, —C(O)NH(C1-C3 alkyl) or —C(O)N(C1-C3 alkyl)².

Certain embodiments of the invention include such compounds or salts wherein Y¹ and Y² join to form piperidine, azepane, azocane, thiazepine, diazepane, oxazepane, azetidine, pyrrolidine, piperazine bound to a fused ring via nitrogen or thiomorpholine.

Certain embodiments of the invention include such compounds or salts wherein two R7 on the same atom join to form a spirocyclic ring selected from C3-C6 cycloalkyl and heterocycle, where said spirocyclic ring is optionally substituted with one or more substituents selected from oxo (═O), halogen, hydroxy, C1-C3 alkyl and —O—(C1-C3 alkyl).

Certain embodiments of the invention include such compounds or salts wherein two R⁷ on adjacent atoms join to form a bond or a fused ring selected from C3-C6 cycloalkyl optionally substituted with 1-4 R¹; heteroaryl optionally substituted with 1-4 R¹; aryl optionally substituted with 1-4 R⁸, and heterocycle optionally substituted with 1-4 R⁸.

Certain embodiments of the invention include such compounds or salts wherein two R⁷ on non-adjacent atoms join to form a 1-2 carbon bridge.

Certain embodiments of the invention include such compounds or salts wherein one R¹⁰ is hydrogen, C1-C3 alkyl or halogen, and another R¹⁰ joins with R7 to form a heterocyclic ring.

Certain embodiments of the invention include such compounds or salts wherein two R¹⁰ join to form a heterocyclic ring.

Certain embodiments of the invention include such compounds or salts wherein each R¹⁰ is independently hydrogen, C1-C3 alkyl or halogen.

Certain embodiments of the invention include such compounds or salts wherein q is 1. It is preferred that q is 1.

Non-limiting examples of compounds of Formula (I) are selected from the group consisting of.

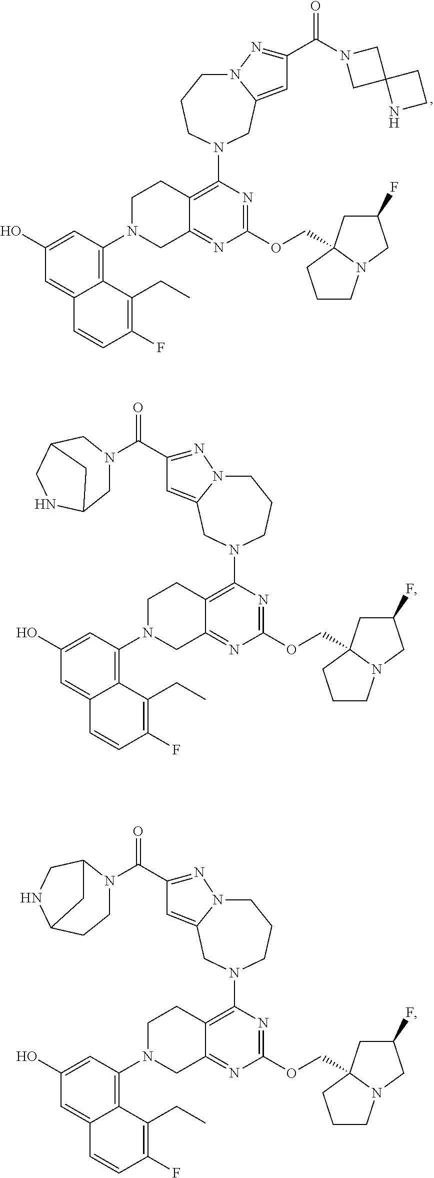 [SMILES: CCc1c(F)ccc2cc(O)cc(N3CCc4c(nc(OC[C@@]56CCCN5C[C@H](F)C6)nc4N4CCCn5nc(C(=O)N6CC7(CCN7)C6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CNC(C7)C6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CCC7CC6CN7)cc5C4)C3)c12]

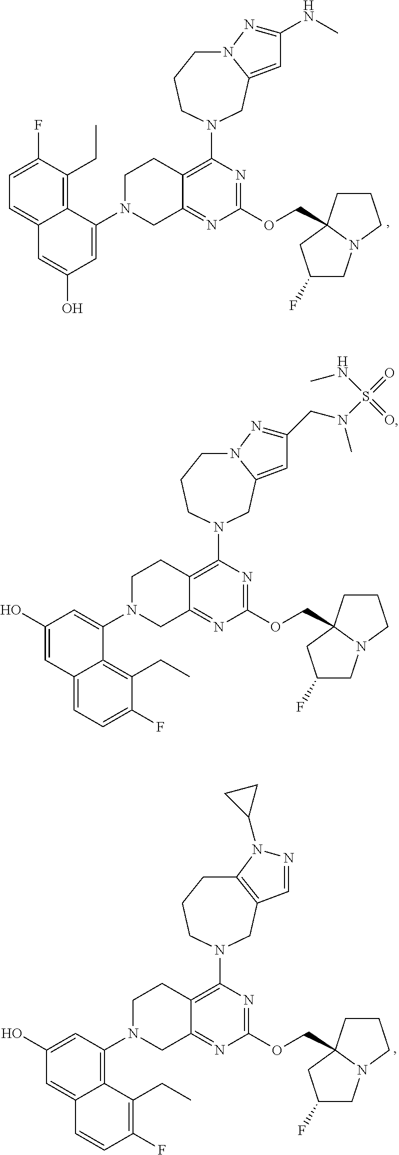 [SMILES: CCc1c(F)ccc2cc(O)cc(N3CCc4c(nc(OC[C@@]56CCCN5C[C@H](F)C6)nc4N4CCCn5nc(NC)cc5C4)C3)c12 ; CCc1c(F)ccc2cc(O)cc(N3CCc4c(nc(OC[C@@]56CCCN5C[C@H](F)C6)nc4N4CCCn5nc(CN(C)S(=O)(=O)NC)cc5C4)C3)c12 ; CCc1c(F)ccc2cc(O)cc(N3CCc4c(nc(OC[C@@]56CCCN5C[C@H](F)C6)nc4N4CCCc5c(cnn5C5CC5)C4)C3)c12]

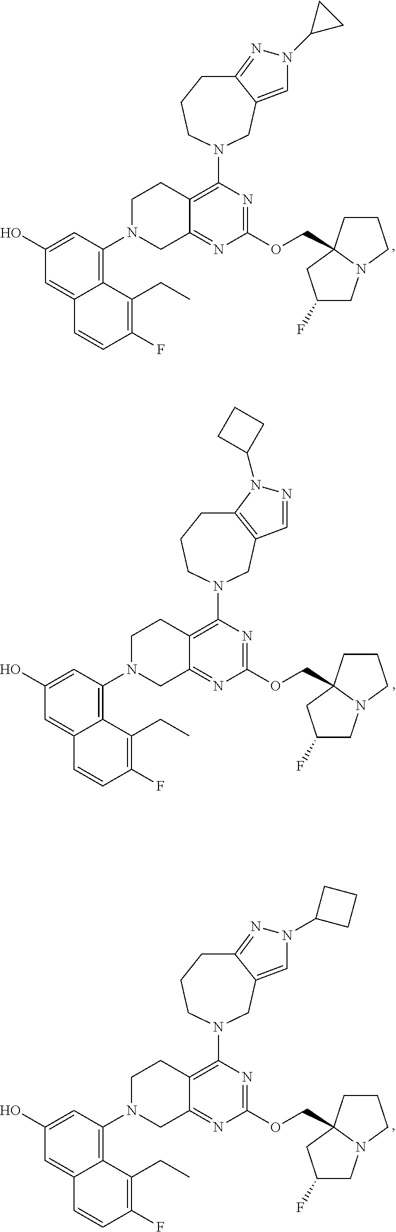 [SMILES: CCc1c(F)ccc2cc(O)cc(N3CCc4c(nc(OC[C@@]56CCCN5C[C@H](F)C6)nc4N4CCCc5nn(C6CC6)cc5C4)C3)c12 ; CCc1c(F)ccc2cc(O)cc(N3CCc4c(nc(OC[C@@]56CCCN5C[C@H](F)C6)nc4N4CCCc5c(cnn5C5CCC5)C4)C3)c12 ; CCc1c(F)ccc2cc(O)cc(N3CCc4c(nc(OC[C@@]56CCCN5C[C@H](F)C6)nc4N4CCCc5nn(C6CCC6)cc5C4)C3)c12]

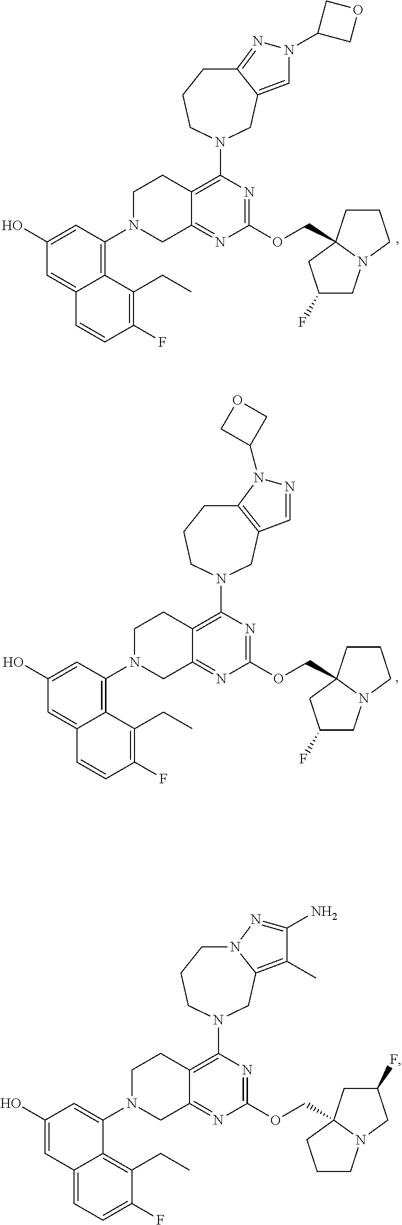 [SMILES: CCc1c(F)ccc2cc(O)cc(N3CCc4c(nc(OC[C@@]56CCCN5C[C@H](F)C6)nc4N4CCCc5nn(C6COC6)cc5C4)C3)c12 ; CCc1c(F)ccc2cc(O)cc(N3CCc4c(nc(OC[C@@]56CCCN5C[C@H](F)C6)nc4N4CCCc5c(cnn5C5COC5)C4)C3)c12 ; CCc1c(F)ccc2cc(O)cc(N3CCc4c(nc(OC[C@@]56CCCN5C[C@H](F)C6)nc4N4CCCn5nc(N)c(C)c5C4)C3)c12]

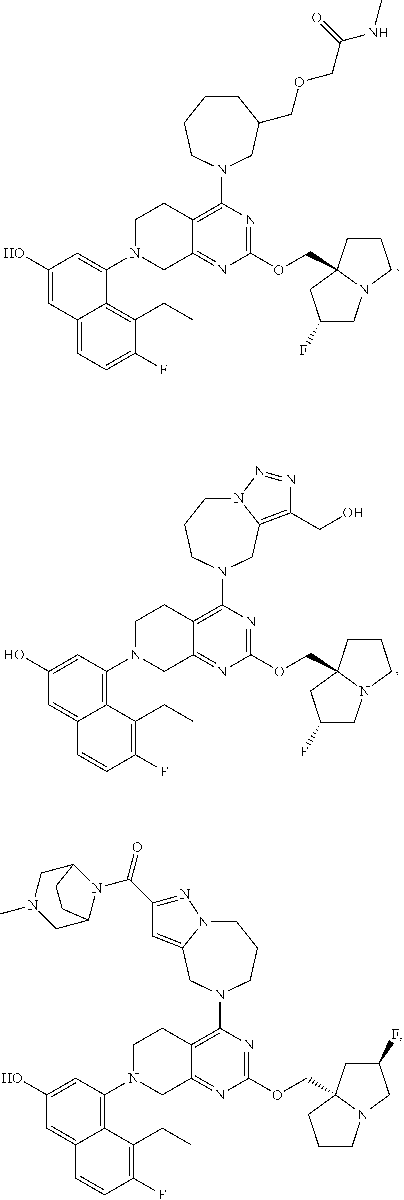 [SMILES: CCc1c(F)ccc2cc(O)cc(N3CCc4c(nc(OC[C@@]56CCCN5C[C@H](F)C6)nc4N4CCCCC(COCC(=O)NC)C4)C3)c12 ; CCc1c(F)ccc2cc(O)cc(N3CCc4c(nc(OC[C@@]56CCCN5C[C@H](F)C6)nc4N4CCCn5nnc(CO)c5C4)C3)c12 ; CCc1c(F)ccc2cc(O)cc(N3CCc4c(nc(OC[C@@]56CCCN5C[C@H](F)C6)nc4N4CCCn5nc(C(=O)N6C7CCC6CN(C)C7)cc5C4)C3)c12]

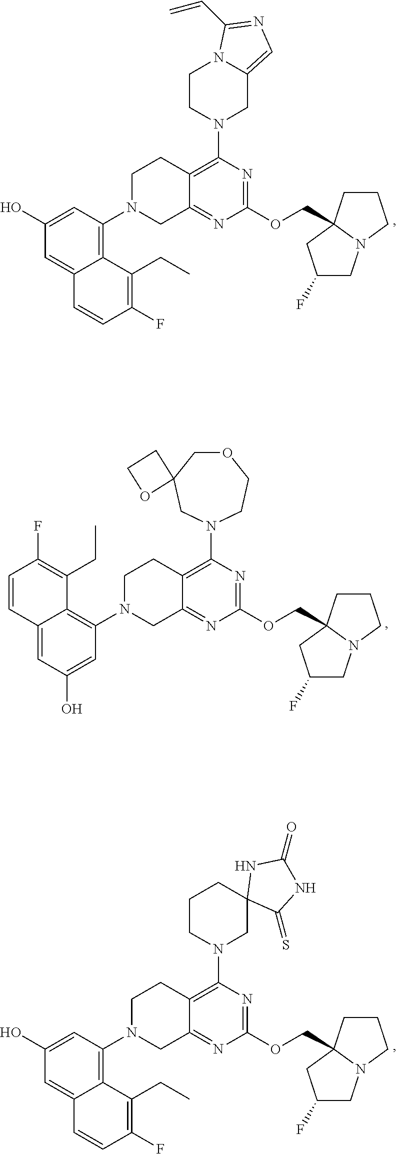 [SMILES: C=Cc1ncc2n1CCN(c1nc(OC[C@@]34CCCN3C[C@H](F)C4)nc3c1CCN(c1cc(O)cc4ccc(F)c(CC)c14)C3)C2 ; CCc1c(F)ccc2cc(O)cc(N3CCc4c(nc(OC[C@@]56CCCN5C[C@H](F)C6)nc4N4CCOCC5(CCO5)C4)C3)c12 ; CCc1c(F)ccc2cc(O)cc(N3CCc4c(nc(OC[C@@]56CCCN5C[C@H](F)C6)nc4N4CCCC5(C4)NC(=O)NC5=S)C3)c12]

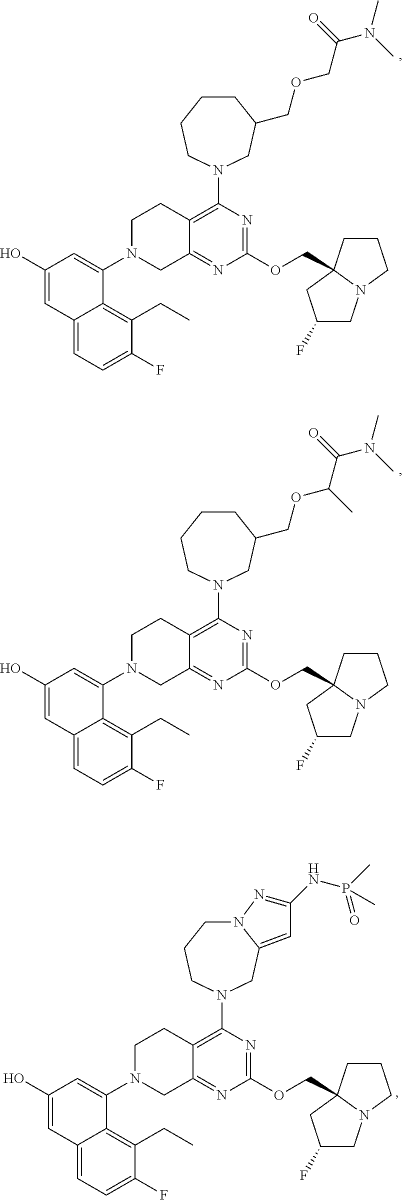 [SMILES: CCc1c(F)ccc2cc(O)cc(N3CCc4c(nc(OC[C@@]56CCCN5C[C@H](F)C6)nc4N4CCCCC(COCC(=O)N(C)C)C4)C3)c12 ; CCc1c(F)ccc2cc(O)cc(N3CCc4c(nc(OC[C@@]56CCCN5C[C@H](F)C6)nc4N4CCCCC(COC(C)C(=O)N(C)C)C4)C3)c12 ; CCc1c(F)ccc2cc(O)cc(N3CCc4c(nc(OC[C@@]56CCCN5C[C@H](F)C6)nc4N4CCCn5nc(NP(C)(C)=O)cc5C4)C3)c12]

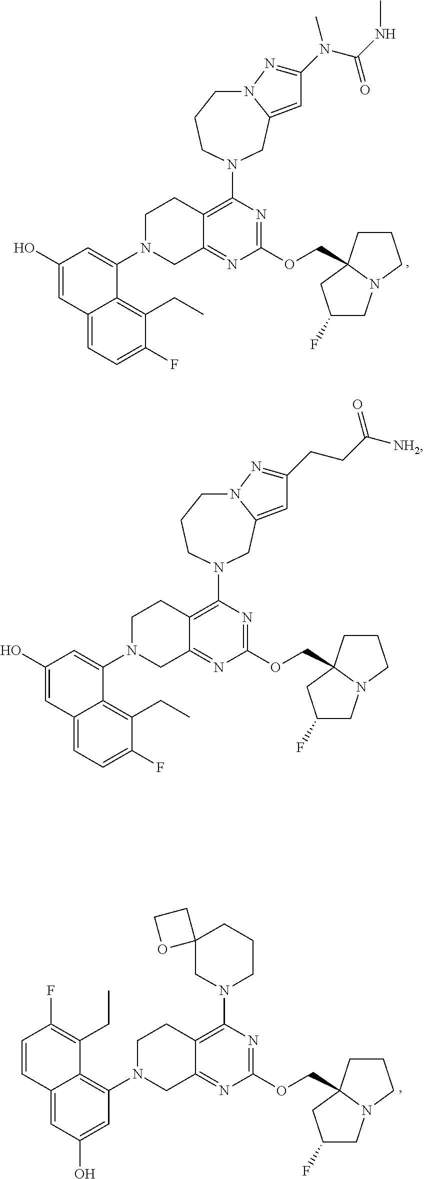 [SMILES: CCc1c(F)ccc2cc(O)cc(N3CCc4c(nc(OC[C@@]56CCCN5C[C@H](F)C6)nc4N4CCCn5nc(N(C)C(=O)NC)cc5C4)C3)c12 ; CCc1c(F)ccc2cc(O)cc(N3CCc4c(nc(OC[C@@]56CCCN5C[C@H](F)C6)nc4N4CCCn5nc(CCC(N)=O)cc5C4)C3)c12 ; CCc1c(F)ccc2cc(O)cc(N3CCc4c(nc(OC[C@@]56CCCN5C[C@H](F)C6)nc4N4CCCC5(CCO5)C4)C3)c12]

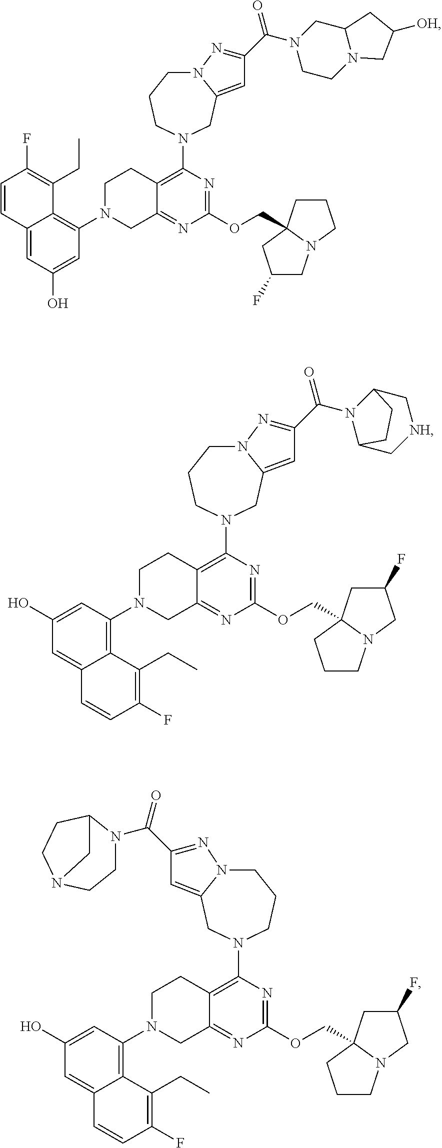 [SMILES: CCc1c(F)ccc2cc(O)cc(N3CCc4c(nc(OC[C@@]56CCCN5C[C@H](F)C6)nc4N4CCCn5nc(C(=O)N6CCN7CC(O)CC7C6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6C7CCC6CNC7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CCN7CCC6C7)cc5C4)C3)c12]

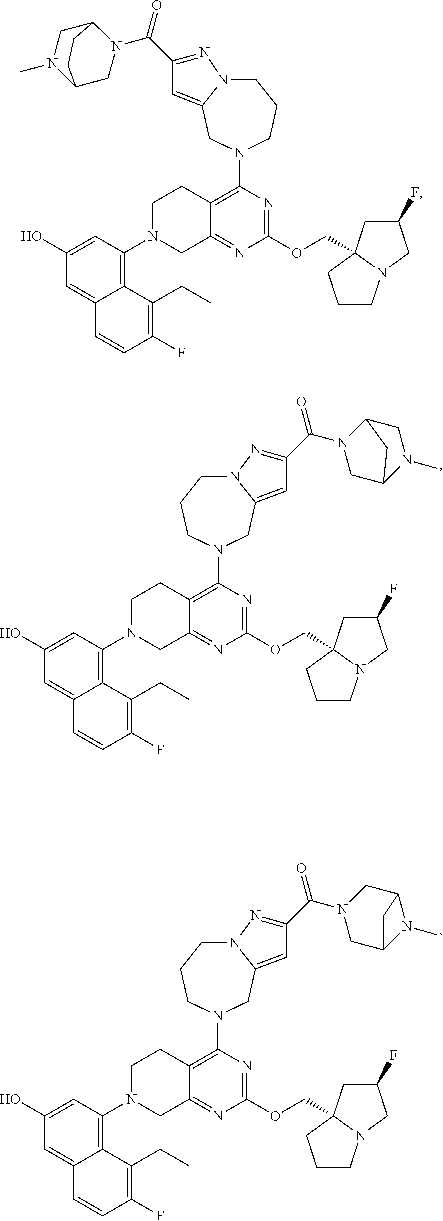 [SMILES: CCc1c(F)ccc2cc(O)cc(N3CCc4c(nc(OC[C@@]56CCCN5C[C@H](F)C6)nc4N4CCCn5nc(C(=O)N6CC7CCC6CN7C)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CC6CN7C)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CC(C6)N7C)cc5C4)C3)c12]

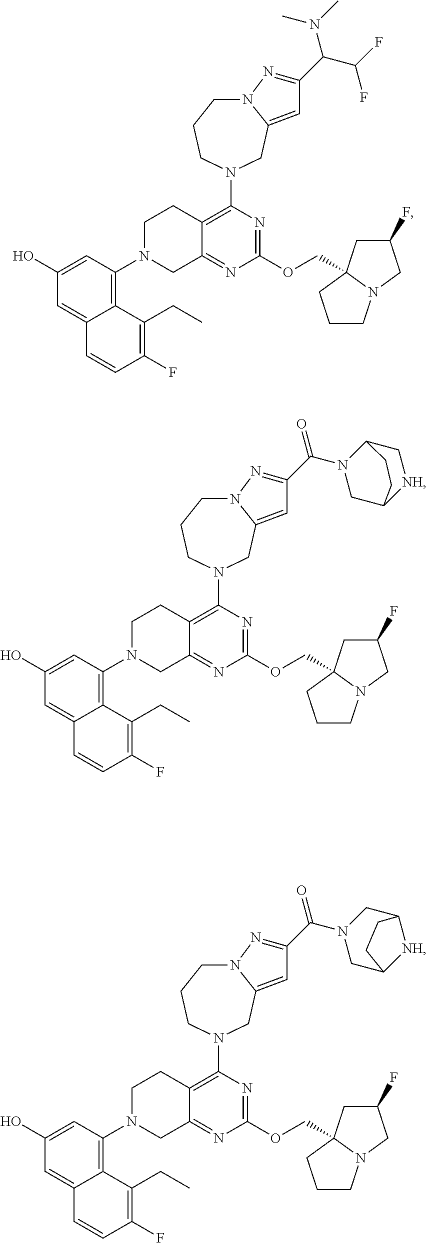 [SMILES: CCc1c(F)ccc2cc(O)cc(N3CCc4c(nc(OC[C@@]56CCCN5C[C@H](F)C6)nc4N4CCCn5nc(C(C(F)F)N(C)C)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CCC6CN7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CCC(C6)N7)cc5C4)C3)c12]

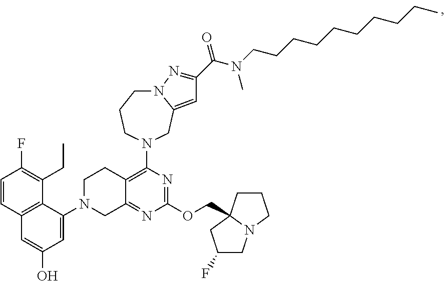 [SMILES: CCCCCCCCCCN(C)C(=O)c1cc2n(n1)CCCN(c1nc(OC[C@@]34CCCN3C[C@H](F)C4)nc3c1CCN(c1cc(O)cc4ccc(F)c(CC)c14)C3)C2]

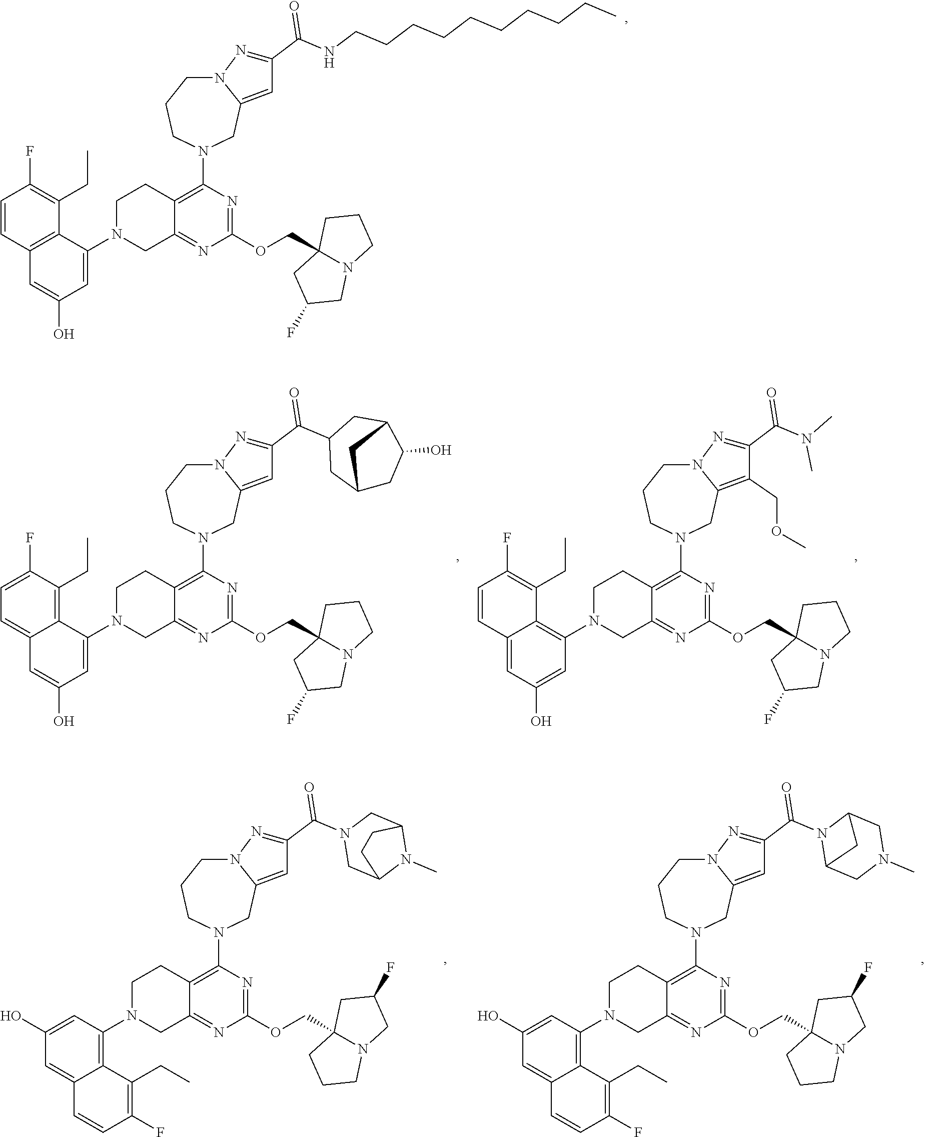

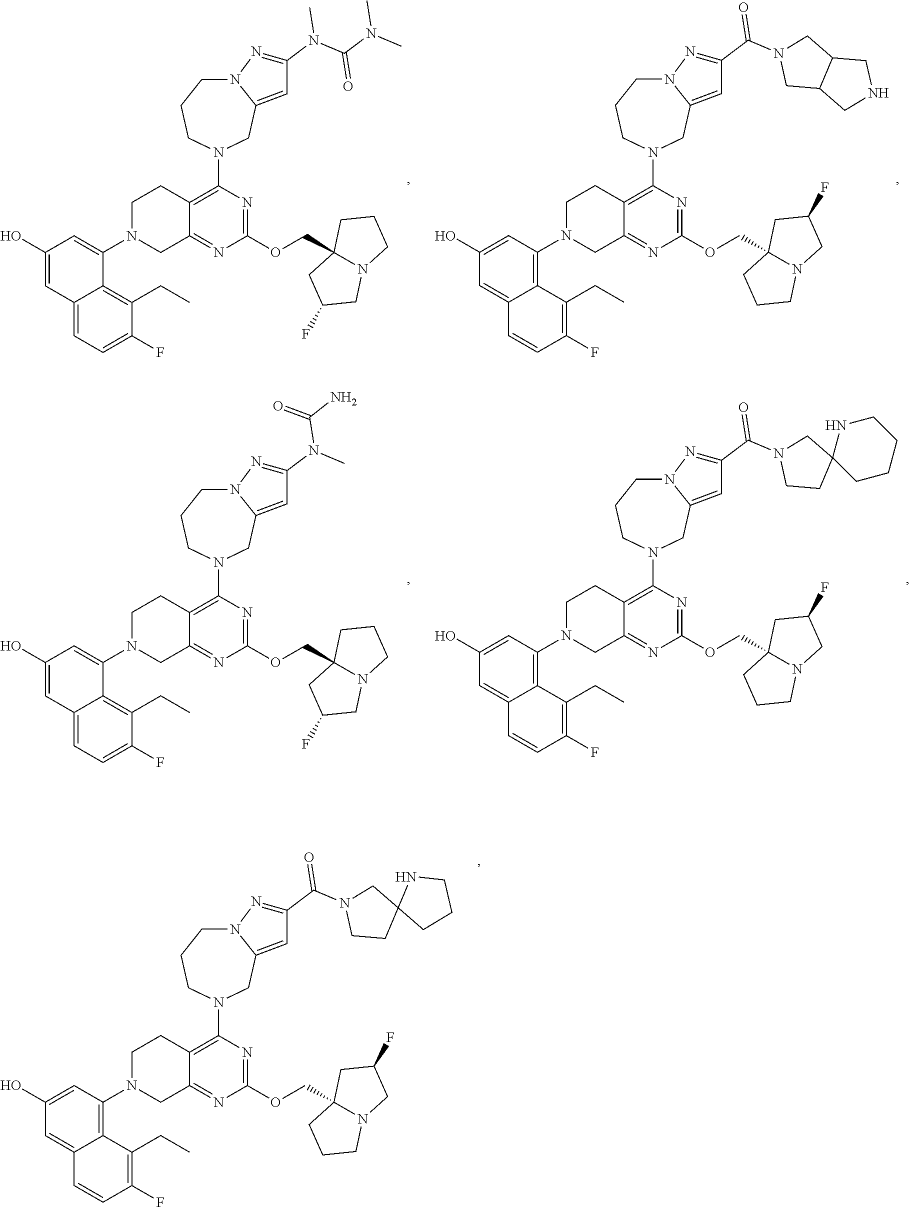

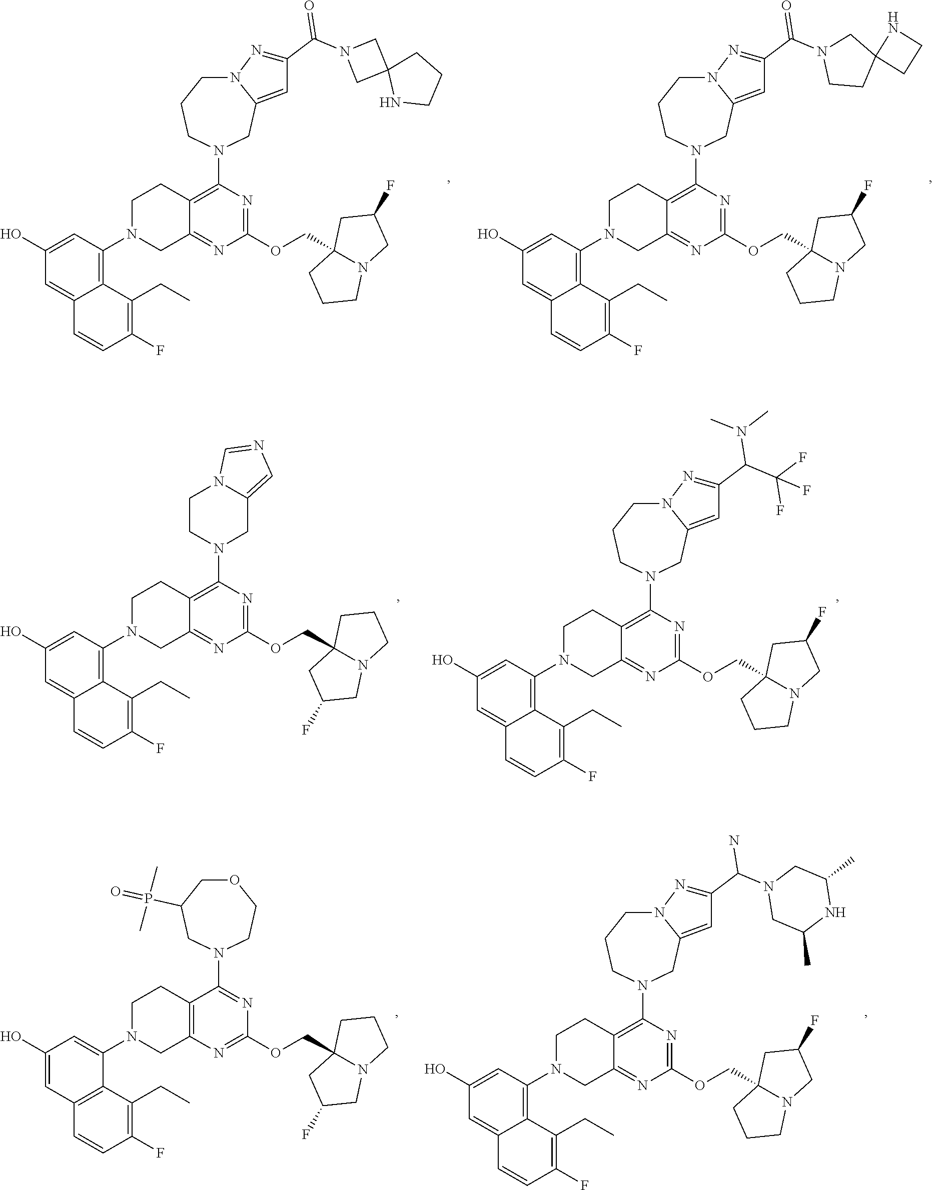

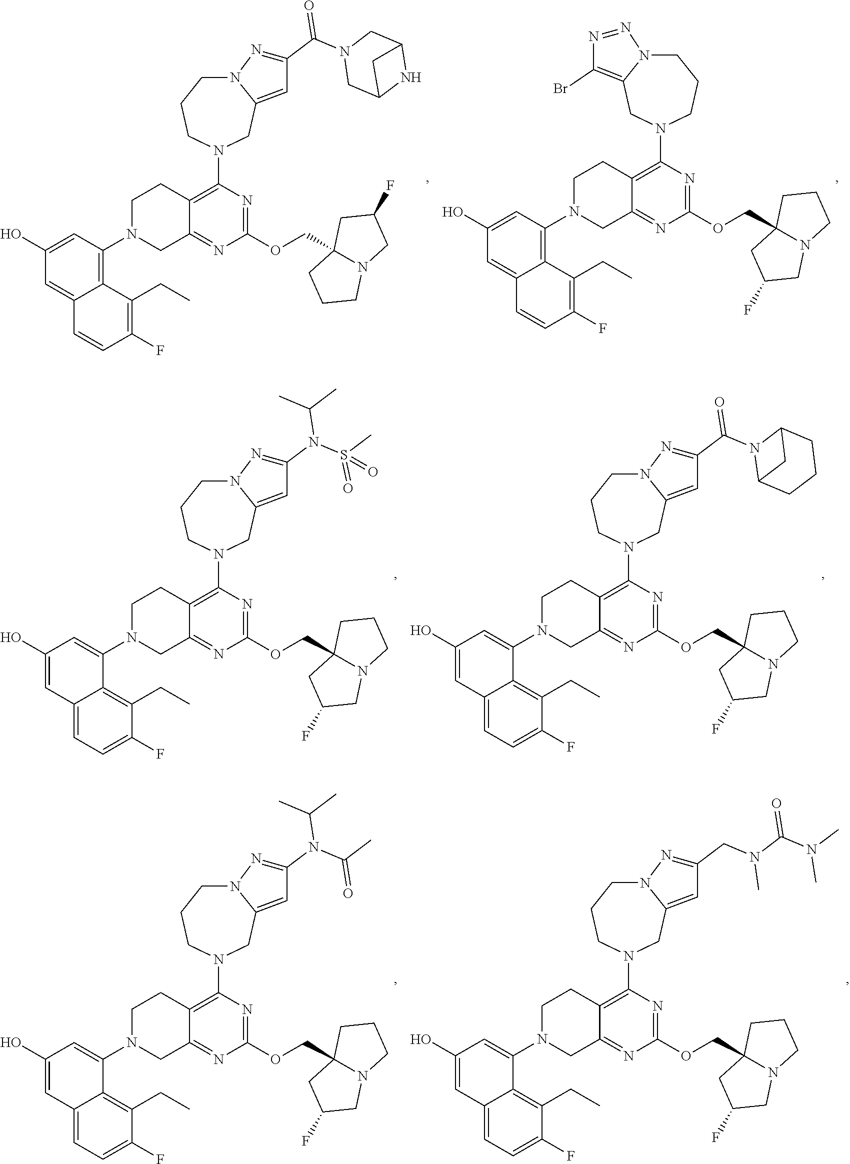

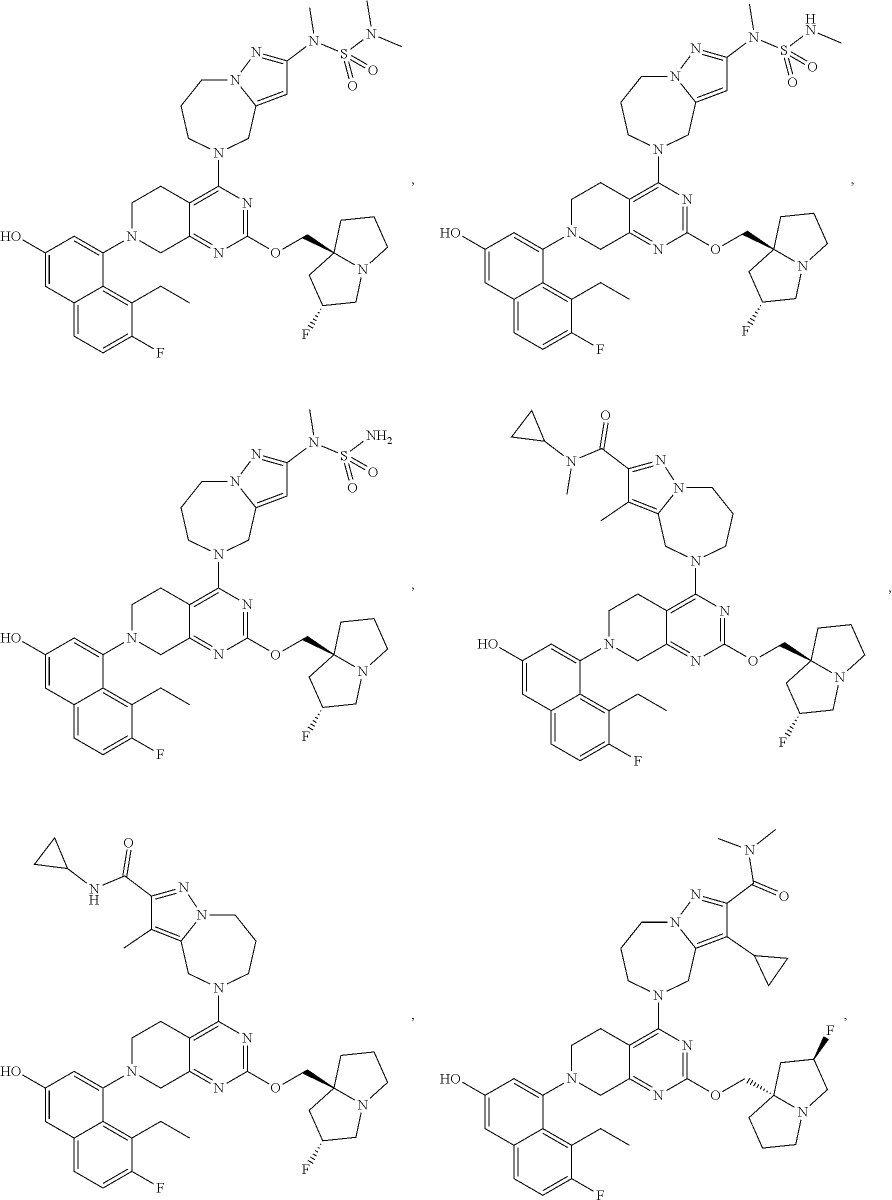

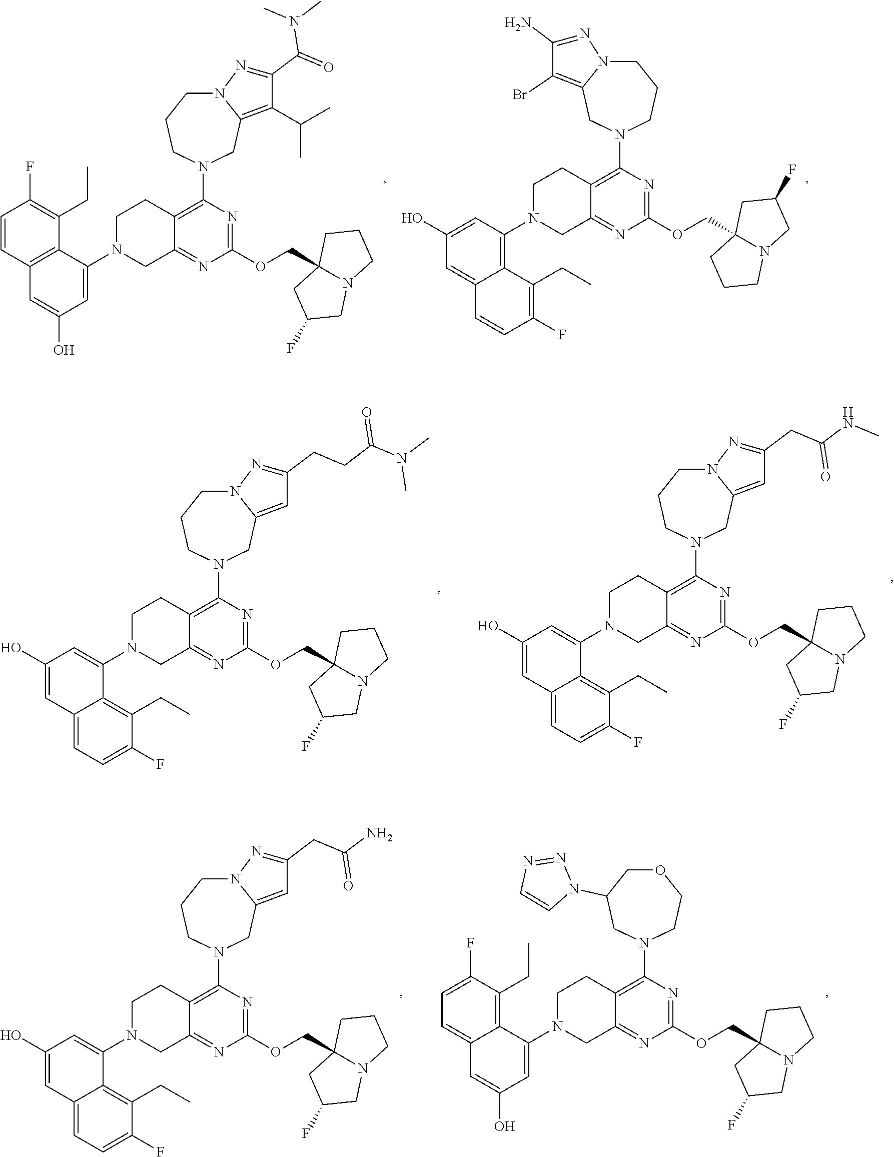

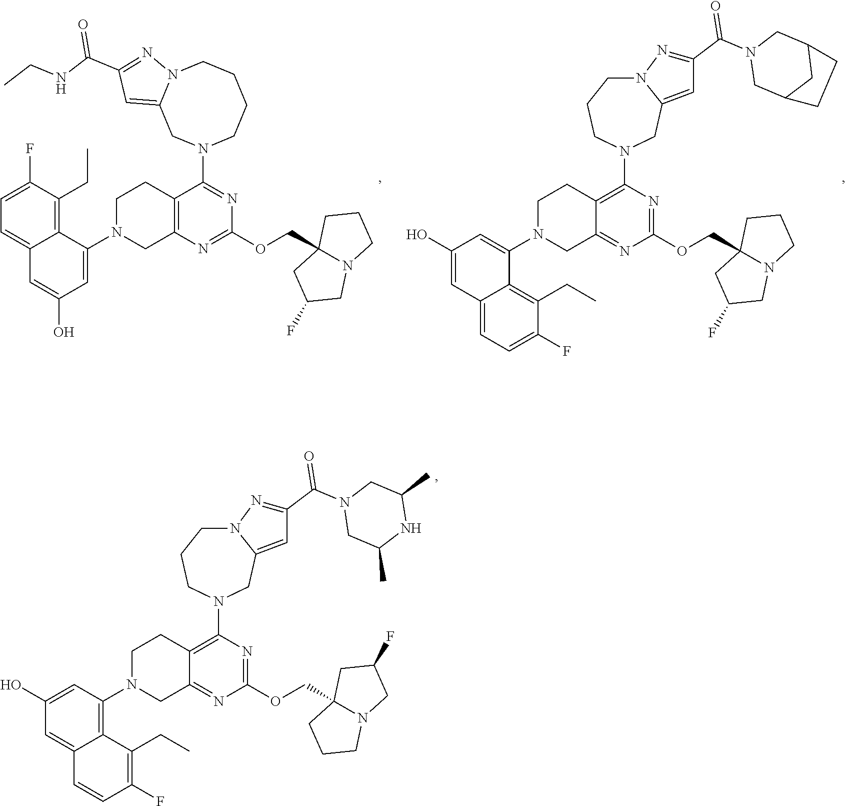 [SMILES: CCNC(=O)c1cc2n(n1)CCCCN(c1nc(OC[C@@]34CCCN3C[C@H](F)C4)nc3c1CCN(c1cc(O)cc4ccc(F)c(CC)c14)C3)C2 ; CCc1c(F)ccc2cc(O)cc(N3CCc4c(nc(OC[C@@]56CCCN5C[C@H](F)C6)nc4N4CCCn5nc(C(=O)N6CC7CCC(C7)C6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6C[C@@H](C)N[C@@H](C)C6)cc5C4)C3)c12]

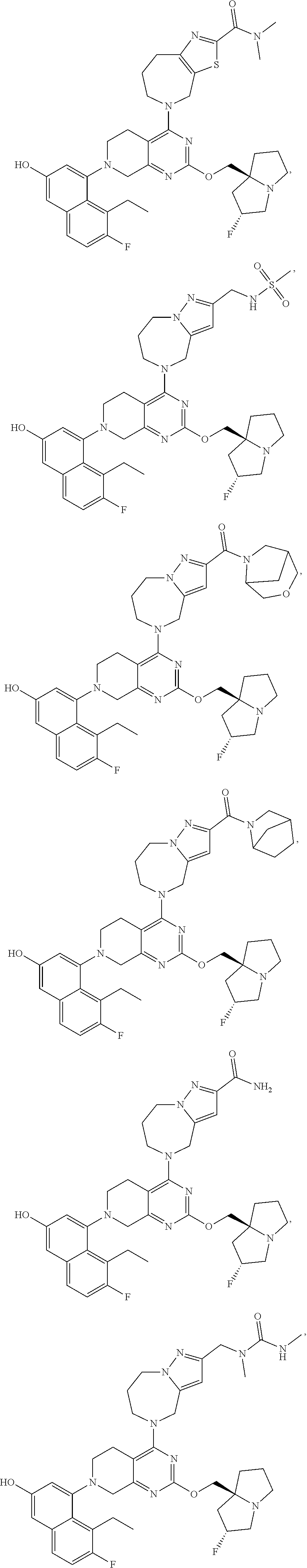

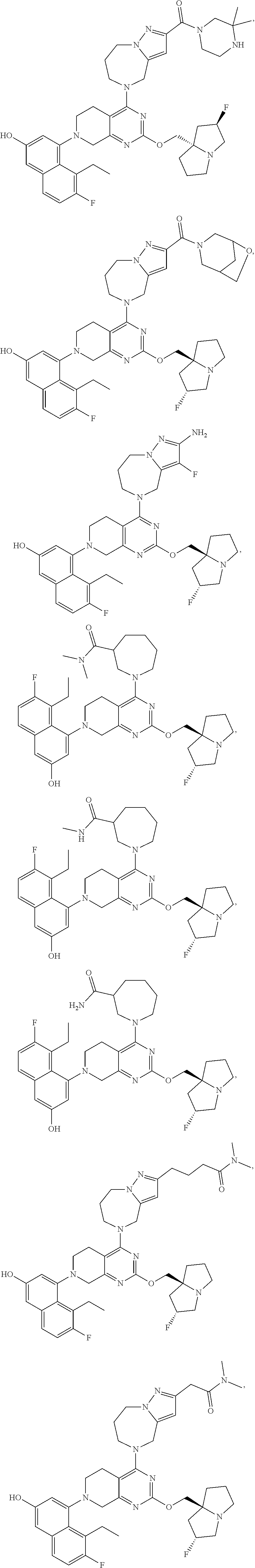

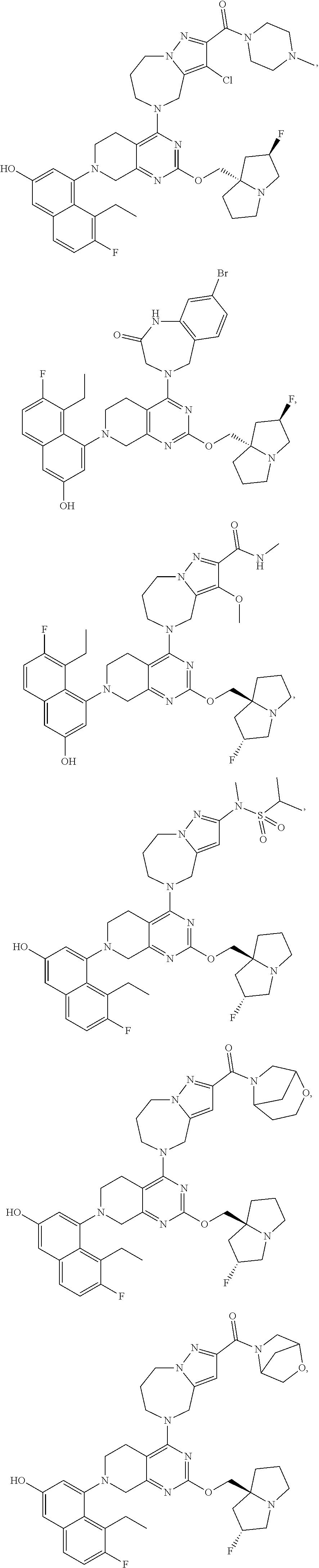

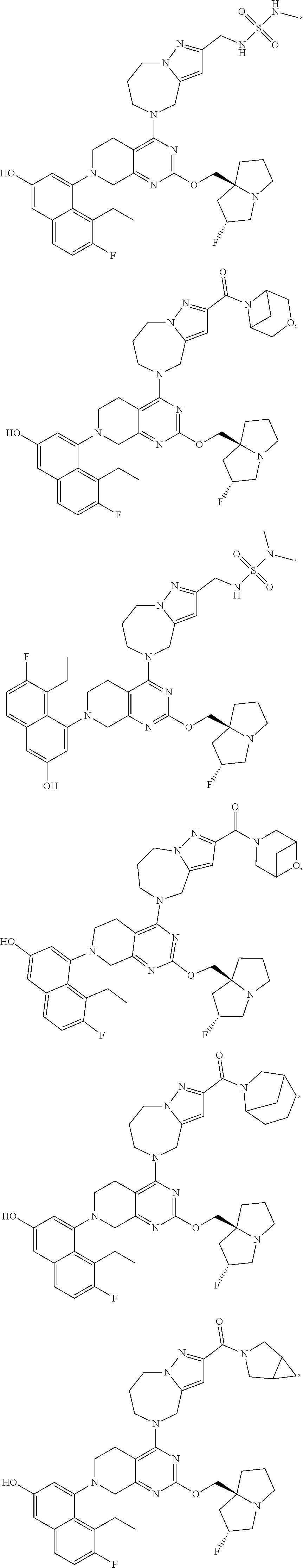

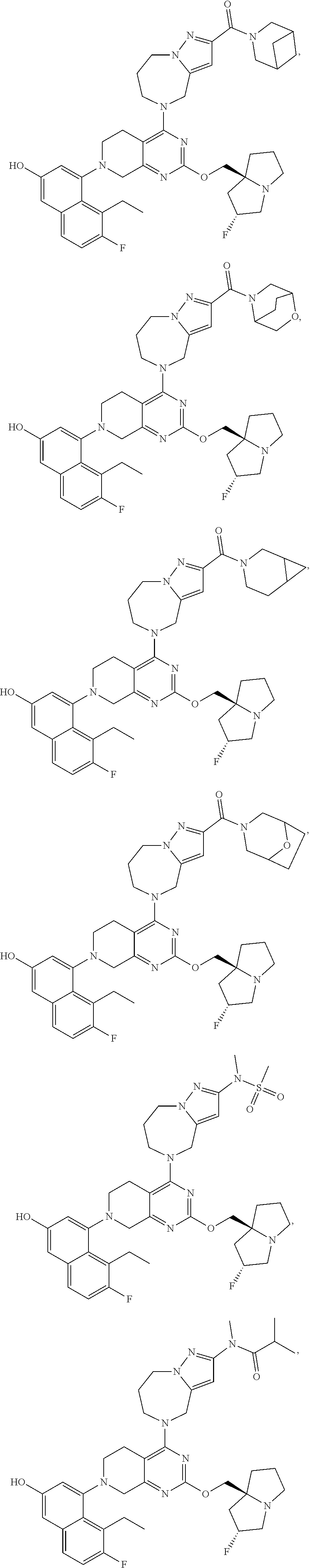

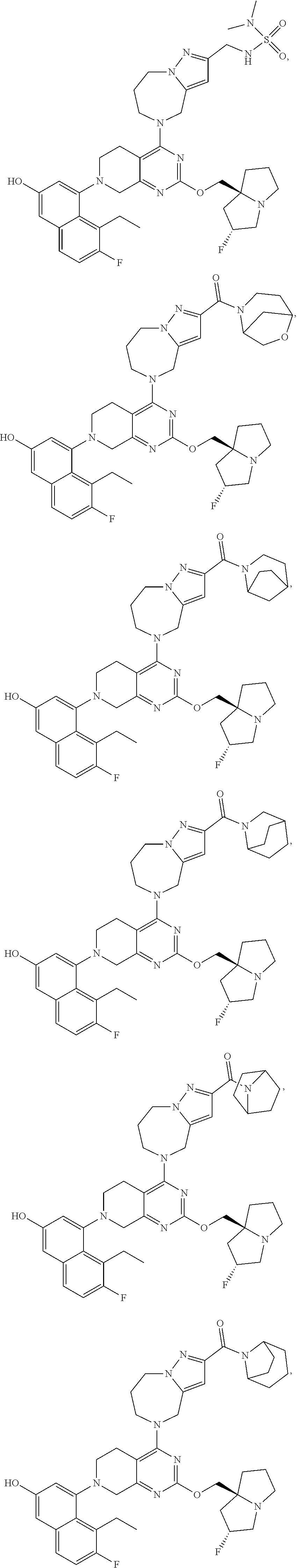

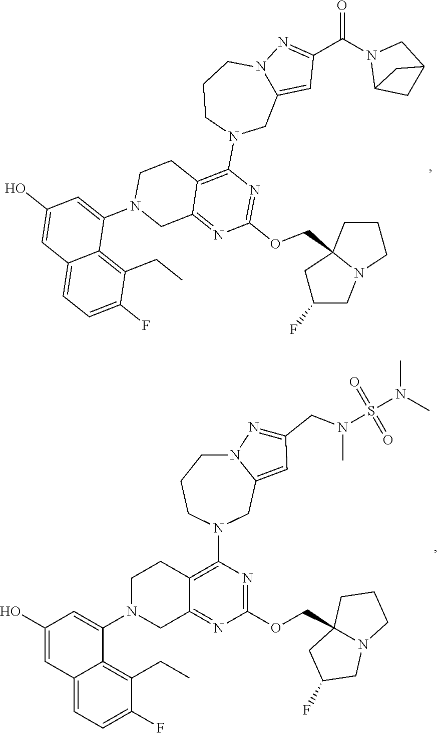 [SMILES: CCc1c(F)ccc2cc(O)cc(N3CCc4c(nc(OC[C@@]56CCCN5C[C@H](F)C6)nc4N4CCCn5nc(C(=O)N6CC7CC6C7)cc5C4)C3)c12 ; CCc1c(F)ccc2cc(O)cc(N3CCc4c(nc(OC[C@@]56CCCN5C[C@H](F)C6)nc4N4CCCn5nc(CN(C)S(=O)(=O)N(C)C)cc5C4)C3)c12]

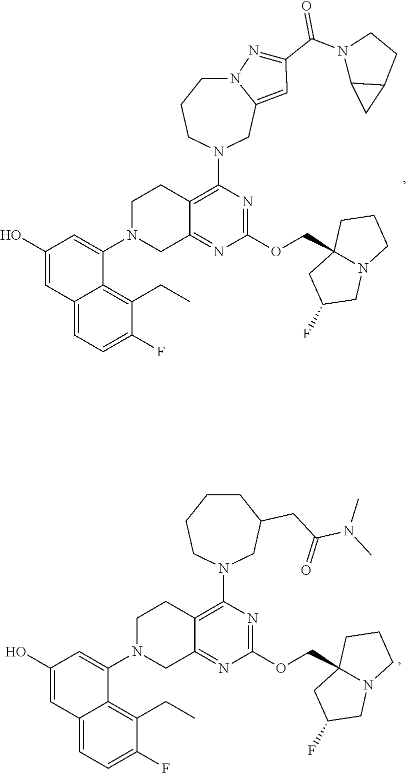 [SMILES: CCc1c(F)ccc2cc(O)cc(N3CCc4c(nc(OC[C@@]56CCCN5C[C@H](F)C6)nc4N4CCCn5nc(C(=O)N6CCC7CC76)cc5C4)C3)c12 ; CCc1c(F)ccc2cc(O)cc(N3CCc4c(nc(OC[C@@]56CCCN5C[C@H](F)C6)nc4N4CCCCC(CC(=O)N(C)C)C4)C3)c12]

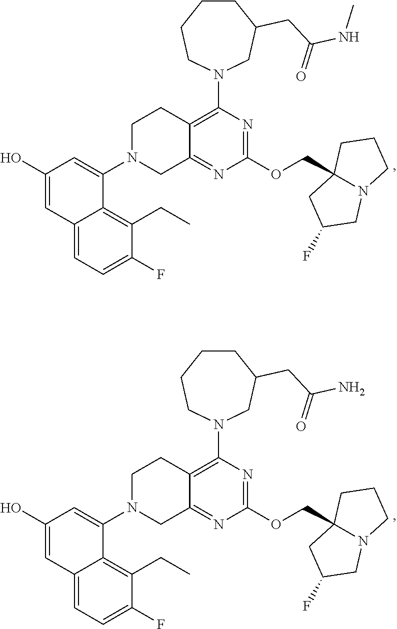 [SMILES: CCc1c(F)ccc2cc(O)cc(N3CCc4c(nc(OC[C@@]56CCCN5C[C@H](F)C6)nc4N4CCCCC(CC(=O)NC)C4)C3)c12 ; CCc1c(F)ccc2cc(O)cc(N3CCc4c(nc(OC[C@@]56CCCN5C[C@H](F)C6)nc4N4CCCCC(CC(N)=O)C4)C3)c12]

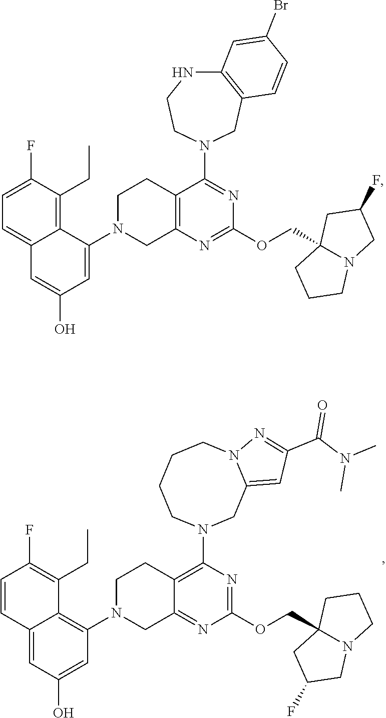 [SMILES: CCc1c(F)ccc2cc(O)cc(N3CCc4c(nc(OC[C@@]56CCCN5C[C@H](F)C6)nc4N4CCNc5cc(Br)ccc5C4)C3)c12 ; CCc1c(F)ccc2cc(O)cc(N3CCc4c(nc(OC[C@@]56CCCN5C[C@H](F)C6)nc4N4CCCCn5nc(C(=O)N(C)C)cc5C4)C3)c12]

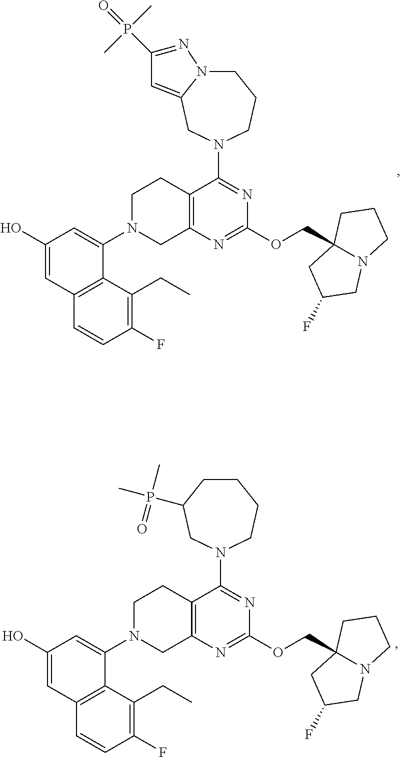 [SMILES: CCc1c(F)ccc2cc(O)cc(N3CCc4c(nc(OC[C@@]56CCCN5C[C@H](F)C6)nc4N4CCCn5nc(P(C)(C)=O)cc5C4)C3)c12 ; CCc1c(F)ccc2cc(O)cc(N3CCc4c(nc(OC[C@@]56CCCN5C[C@H](F)C6)nc4N4CCCCC(P(C)(C)=O)C4)C3)c12]

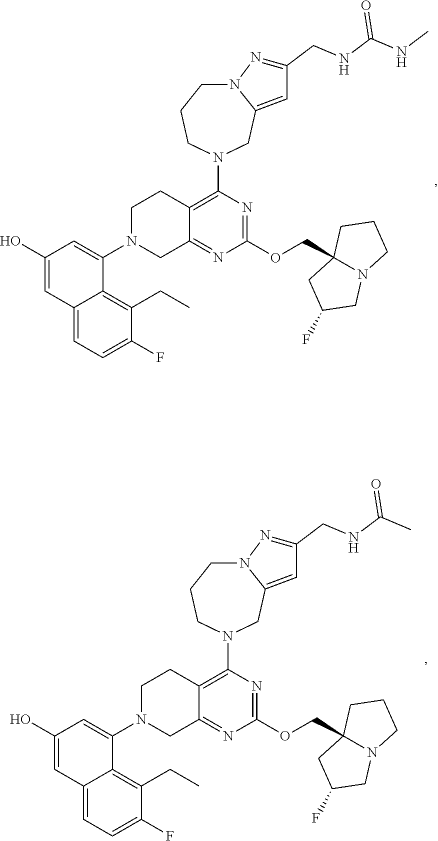 [SMILES: CCc1c(F)ccc2cc(O)cc(N3CCc4c(nc(OC[C@@]56CCCN5C[C@H](F)C6)nc4N4CCCn5nc(CNC(=O)NC)cc5C4)C3)c12 ; CCc1c(F)ccc2cc(O)cc(N3CCc4c(nc(OC[C@@]56CCCN5C[C@H](F)C6)nc4N4CCCn5nc(CNC(C)=O)cc5C4)C3)c12]

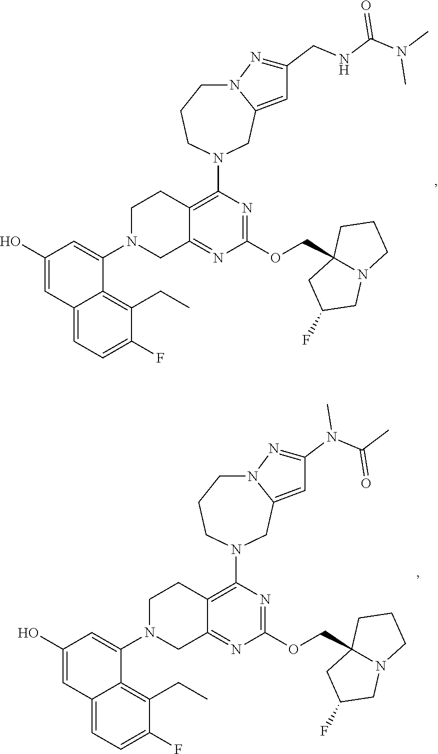 [SMILES: CCc1c(F)ccc2cc(O)cc(N3CCc4c(nc(OC[C@@]56CCCN5C[C@H](F)C6)nc4N4CCCn5nc(CNC(=O)N(C)C)cc5C4)C3)c12 ; CCc1c(F)ccc2cc(O)cc(N3CCc4c(nc(OC[C@@]56CCCN5C[C@H](F)C6)nc4N4CCCn5nc(N(C)C(C)=O)cc5C4)C3)c12]

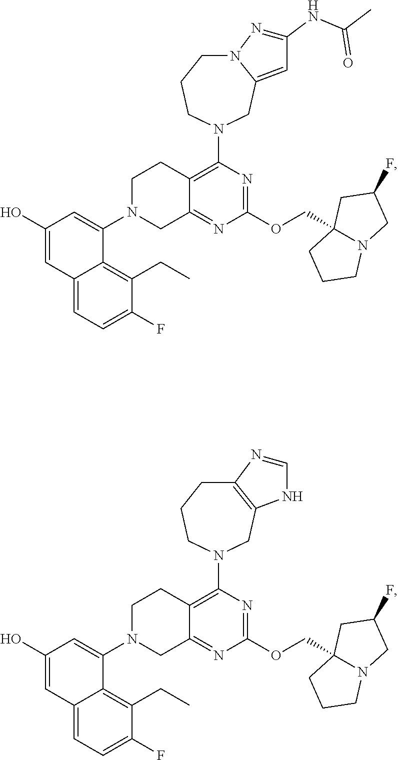 [SMILES: CCc1c(F)ccc2cc(O)cc(N3CCc4c(nc(OC[C@@]56CCCN5C[C@H](F)C6)nc4N4CCCn5nc(NC(C)=O)cc5C4)C3)c12 ; CCc1c(F)ccc2cc(O)cc(N3CCc4c(nc(OC[C@@]56CCCN5C[C@H](F)C6)nc4N4CCCc5nc[nH]c5C4)C3)c12]

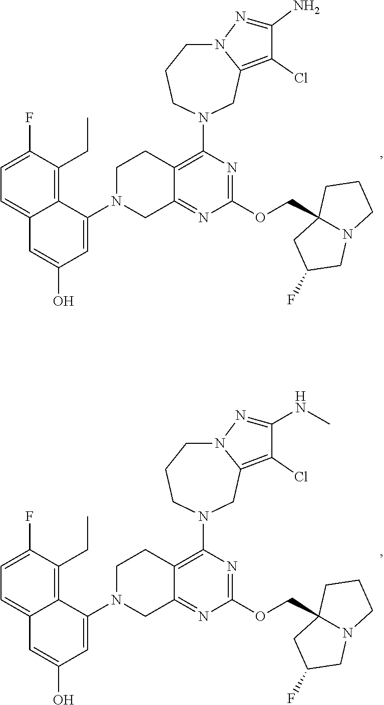 [SMILES: CCc1c(F)ccc2cc(O)cc(N3CCc4c(nc(OC[C@@]56CCCN5C[C@H](F)C6)nc4N4CCCn5nc(N)c(Cl)c5C4)C3)c12 ; CCc1c(F)ccc2cc(O)cc(N3CCc4c(nc(OC[C@@]56CCCN5C[C@H](F)C6)nc4N4CCCn5nc(NC)c(Cl)c5C4)C3)c12]

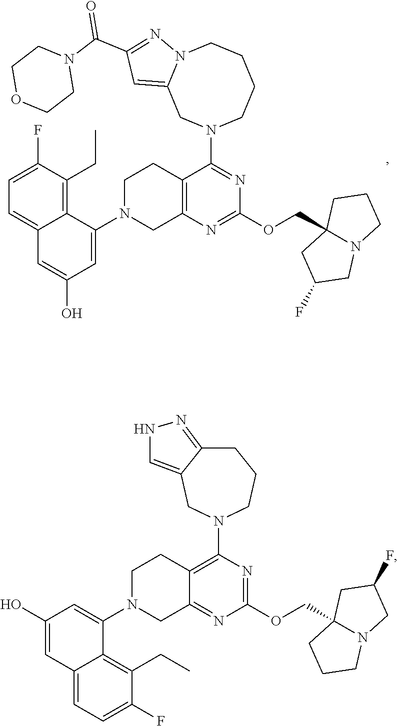 [SMILES: CCc1c(F)ccc2cc(O)cc(N3CCc4c(nc(OC[C@@]56CCCN5C[C@H](F)C6)nc4N4CCCCn5nc(C(=O)N6CCOCC6)cc5C4)C3)c12 ; CCc1c(F)ccc2cc(O)cc(N3CCc4c(nc(OC[C@@]56CCCN5C[C@H](F)C6)nc4N4CCCc5n[nH]cc5C4)C3)c12]

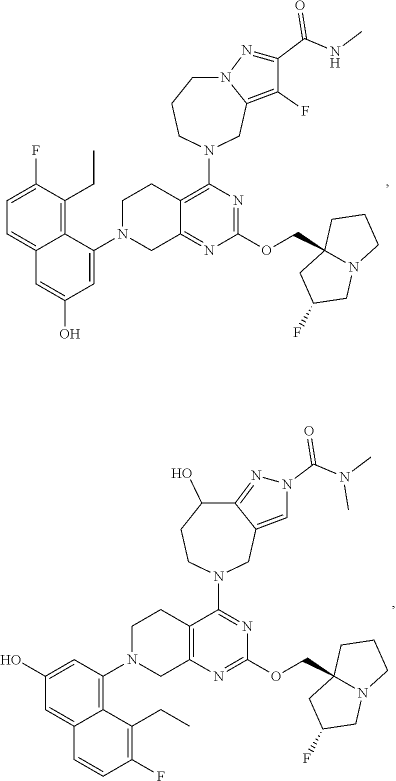 [SMILES: CCc1c(F)ccc2cc(O)cc(N3CCc4c(nc(OC[C@@]56CCCN5C[C@H](F)C6)nc4N4CCCn5nc(C(=O)NC)c(F)c5C4)C3)c12 ; CCc1c(F)ccc2cc(O)cc(N3CCc4c(nc(OC[C@@]56CCCN5C[C@H](F)C6)nc4N4CCC(O)c5nn(C(=O)N(C)C)cc5C4)C3)c12]

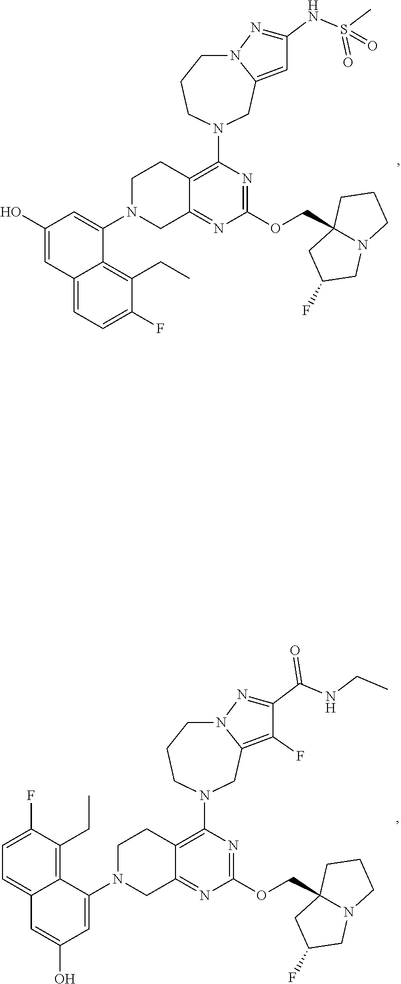 [SMILES: CCc1c(F)ccc2cc(O)cc(N3CCc4c(nc(OC[C@@]56CCCN5C[C@H](F)C6)nc4N4CCCn5nc(NS(C)(=O)=O)cc5C4)C3)c12 ; CCNC(=O)c1nn2c(c1F)CN(c1nc(OC[C@@]34CCCN3C[C@H](F)C4)nc3c1CCN(c1cc(O)cc4ccc(F)c(CC)c14)C3)CCC2]

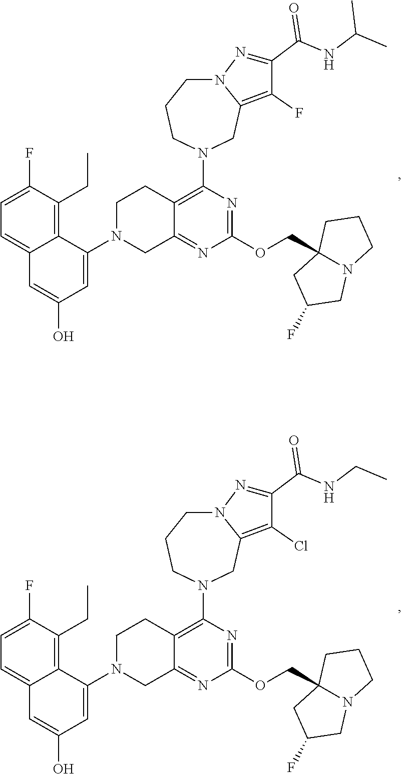 [SMILES: CCc1c(F)ccc2cc(O)cc(N3CCc4c(nc(OC[C@@]56CCCN5C[C@H](F)C6)nc4N4CCCn5nc(C(=O)NC(C)C)c(F)c5C4)C3)c12 ; CCNC(=O)c1nn2c(c1Cl)CN(c1nc(OC[C@@]34CCCN3C[C@H](F)C4)nc3c1CCN(c1cc(O)cc4ccc(F)c(CC)c14)C3)CCC2]

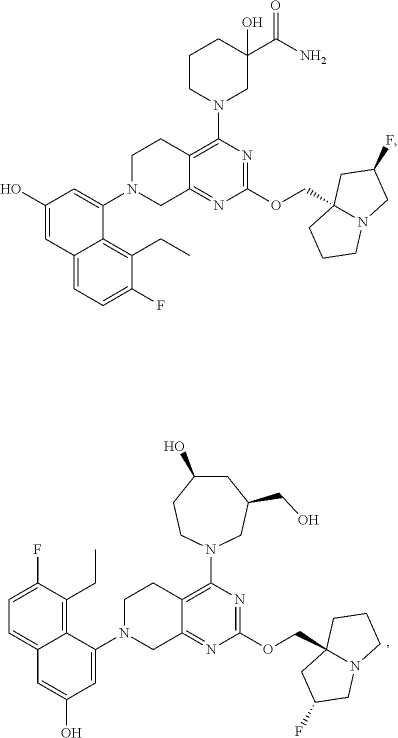 [SMILES: CCc1c(F)ccc2cc(O)cc(N3CCc4c(nc(OC[C@@]56CCCN5C[C@H](F)C6)nc4N4CCCC(O)(C(N)=O)C4)C3)c12 ; CCc1c(F)ccc2cc(O)cc(N3CCc4c(nc(OC[C@@]56CCCN5C[C@H](F)C6)nc4N4CC[C@@H](O)C[C@@H](CO)C4)C3)c12]

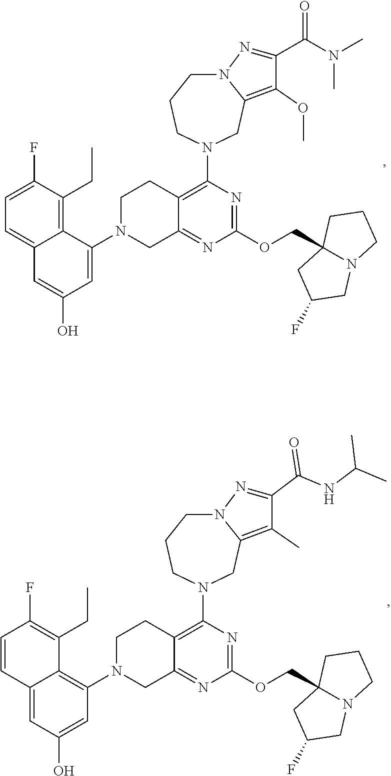 [SMILES: CCc1c(F)ccc2cc(O)cc(N3CCc4c(nc(OC[C@@]56CCCN5C[C@H](F)C6)nc4N4CCCn5nc(C(=O)N(C)C)c(OC)c5C4)C3)c12 ; CCc1c(F)ccc2cc(O)cc(N3CCc4c(nc(OC[C@@]56CCCN5C[C@H](F)C6)nc4N4CCCn5nc(C(=O)NC(C)C)c(C)c5C4)C3)c12]

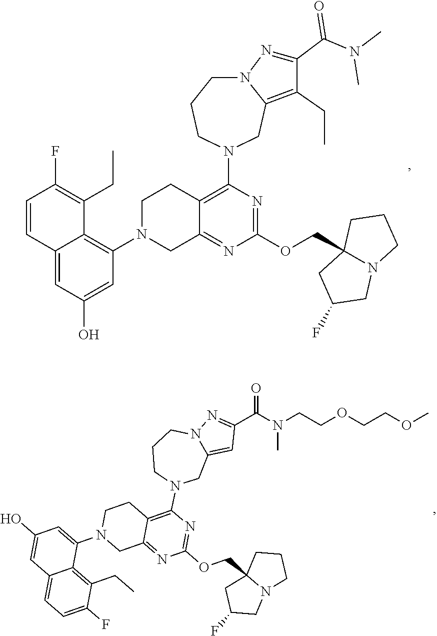 [SMILES: CCc1c(C(=O)N(C)C)nn2c1CN(c1nc(OC[C@@]34CCCN3C[C@H](F)C4)nc3c1CCN(c1cc(O)cc4ccc(F)c(CC)c14)C3)CCC2 ; CCc1c(F)ccc2cc(O)cc(N3CCc4c(nc(OC[C@@]56CCCN5C[C@H](F)C6)nc4N4CCCn5nc(C(=O)N(C)CCOCCOC)cc5C4)C3)c12]

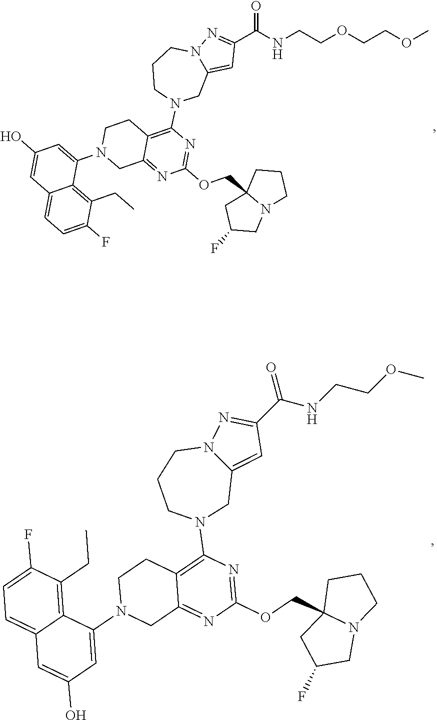 [SMILES: CCc1c(F)ccc2cc(O)cc(N3CCc4c(nc(OC[C@@]56CCCN5C[C@H](F)C6)nc4N4CCCn5nc(C(=O)NCCOCCOC)cc5C4)C3)c12 ; CCc1c(F)ccc2cc(O)cc(N3CCc4c(nc(OC[C@@]56CCCN5C[C@H](F)C6)nc4N4CCCn5nc(C(=O)NCCOC)cc5C4)C3)c12]

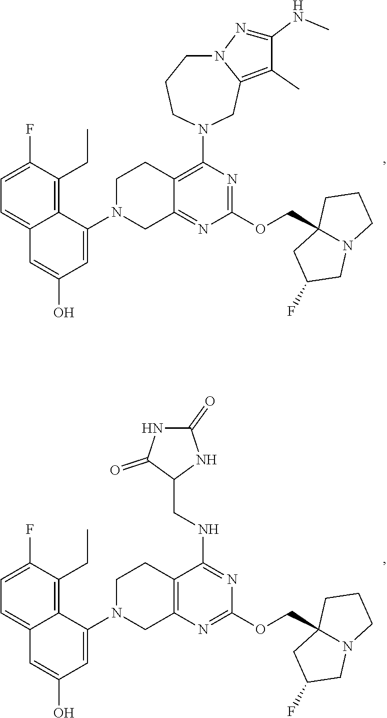 [SMILES: CCc1c(F)ccc2cc(O)cc(N3CCc4c(nc(OC[C@@]56CCCN5C[C@H](F)C6)nc4N4CCCn5nc(NC)c(C)c5C4)C3)c12 ; CCc1c(F)ccc2cc(O)cc(N3CCc4c(nc(OC[C@@]56CCCN5C[C@H](F)C6)nc4NCC4NC(=O)NC4=O)C3)c12]

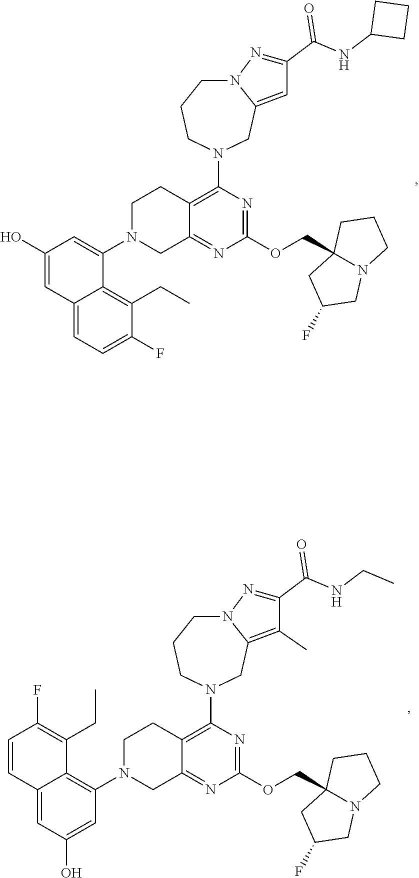 [SMILES: CCc1c(F)ccc2cc(O)cc(N3CCc4c(nc(OC[C@@]56CCCN5C[C@H](F)C6)nc4N4CCCn5nc(C(=O)NC6CCC6)cc5C4)C3)c12 ; CCNC(=O)c1nn2c(c1C)CN(c1nc(OC[C@@]34CCCN3C[C@H](F)C4)nc3c1CCN(c1cc(O)cc4ccc(F)c(CC)c14)C3)CCC2]

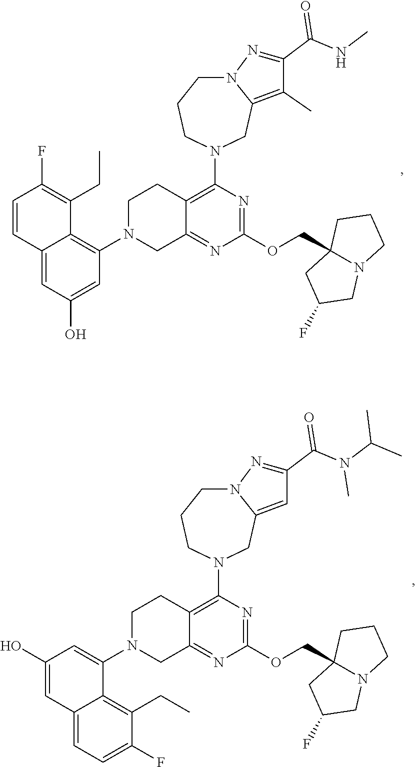 [SMILES: CCc1c(F)ccc2cc(O)cc(N3CCc4c(nc(OC[C@@]56CCCN5C[C@H](F)C6)nc4N4CCCn5nc(C(=O)NC)c(C)c5C4)C3)c12 ; CCc1c(F)ccc2cc(O)cc(N3CCc4c(nc(OC[C@@]56CCCN5C[C@H](F)C6)nc4N4CCCn5nc(C(=O)N(C)C(C)C)cc5C4)C3)c12]

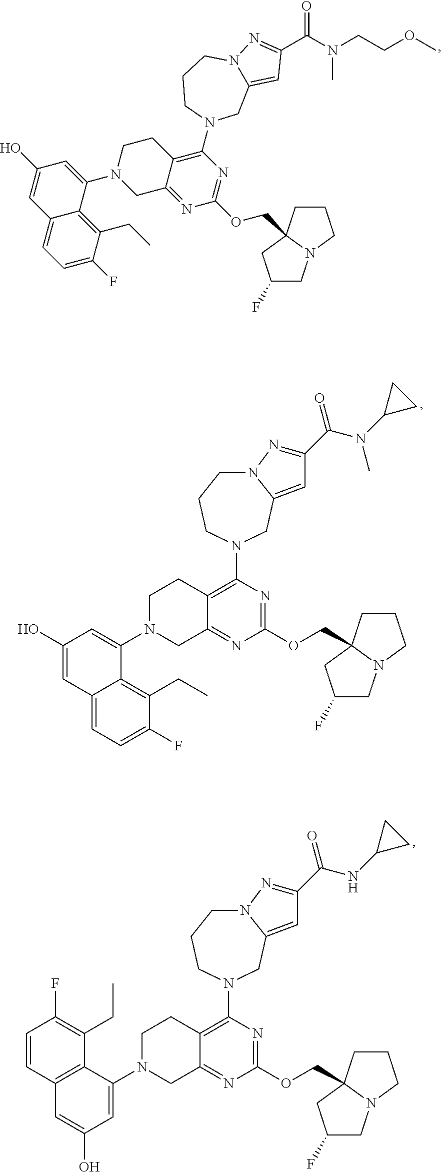 [SMILES: CCc1c(F)ccc2cc(O)cc(N3CCc4c(nc(OC[C@@]56CCCN5C[C@H](F)C6)nc4N4CCCn5nc(C(=O)N(C)CCOC)cc5C4)C3)c12 ; CCc1c(F)ccc2cc(O)cc(N3CCc4c(nc(OC[C@@]56CCCN5C[C@H](F)C6)nc4N4CCCn5nc(C(=O)N(C)C6CC6)cc5C4)C3)c12 ; CCc1c(F)ccc2cc(O)cc(N3CCc4c(nc(OC[C@@]56CCCN5C[C@H](F)C6)nc4N4CCCn5nc(C(=O)NC6CC6)cc5C4)C3)c12]

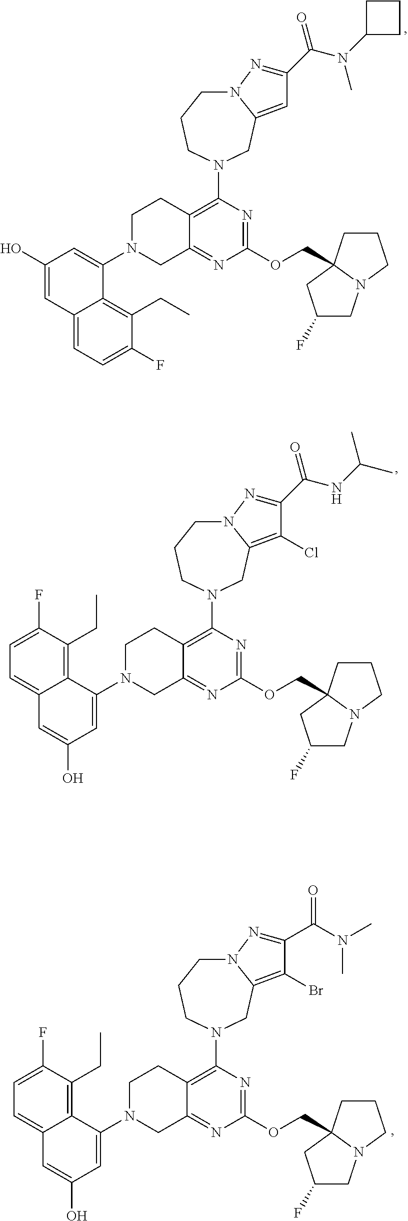 [SMILES: CCc1c(F)ccc2cc(O)cc(N3CCc4c(nc(OC[C@@]56CCCN5C[C@H](F)C6)nc4N4CCCn5nc(C(=O)N(C)C6CCC6)cc5C4)C3)c12 ; CCc1c(F)ccc2cc(O)cc(N3CCc4c(nc(OC[C@@]56CCCN5C[C@H](F)C6)nc4N4CCCn5nc(C(=O)NC(C)C)c(Cl)c5C4)C3)c12 ; CCc1c(F)ccc2cc(O)cc(N3CCc4c(nc(OC[C@@]56CCCN5C[C@H](F)C6)nc4N4CCCn5nc(C(=O)N(C)C)c(Br)c5C4)C3)c12]

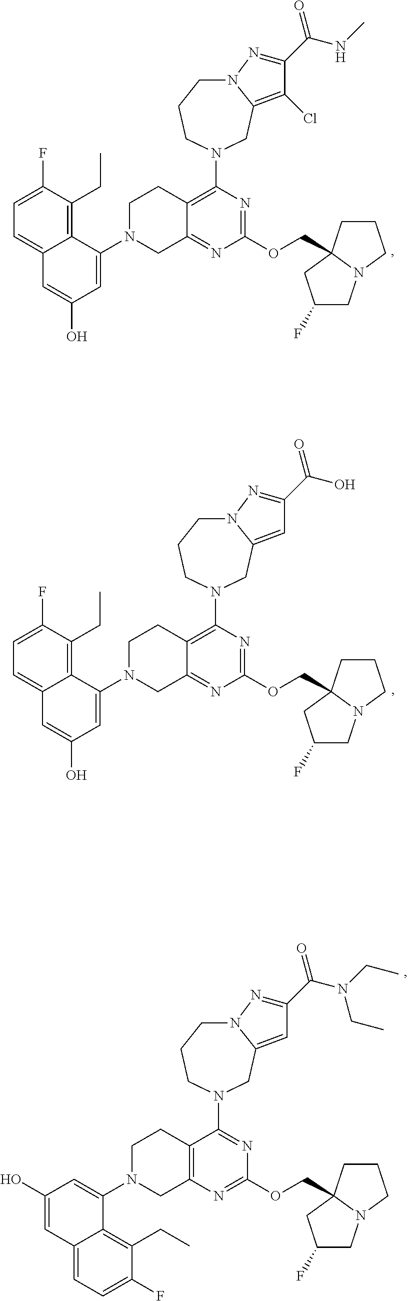 [SMILES: CCc1c(F)ccc2cc(O)cc(N3CCc4c(nc(OC[C@@]56CCCN5C[C@H](F)C6)nc4N4CCCn5nc(C(=O)NC)c(Cl)c5C4)C3)c12 ; CCc1c(F)ccc2cc(O)cc(N3CCc4c(nc(OC[C@@]56CCCN5C[C@H](F)C6)nc4N4CCCn5nc(C(=O)O)cc5C4)C3)c12 ; CCc1c(F)ccc2cc(O)cc(N3CCc4c(nc(OC[C@@]56CCCN5C[C@H](F)C6)nc4N4CCCn5nc(C(=O)N(CC)CC)cc5C4)C3)c12]

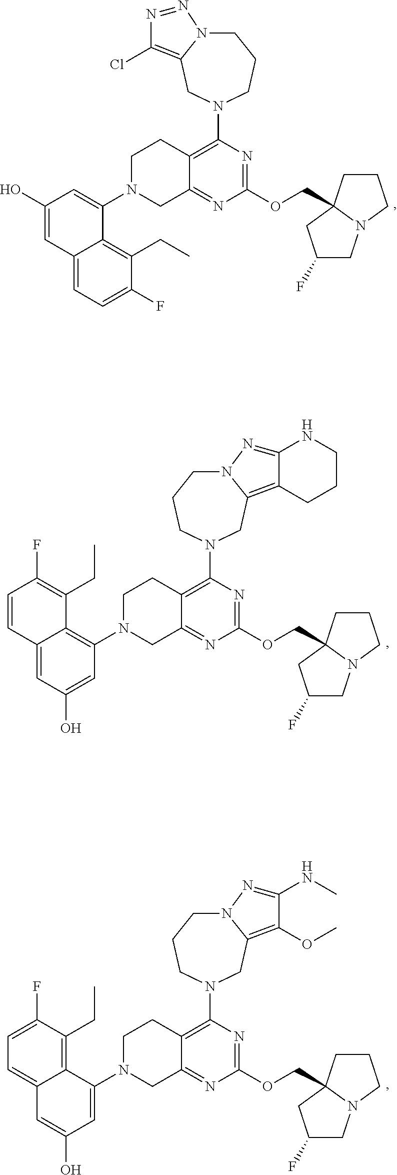 [SMILES: CCc1c(F)ccc2cc(O)cc(N3CCc4c(nc(OC[C@@]56CCCN5C[C@H](F)C6)nc4N4CCCn5nnc(Cl)c5C4)C3)c12 ; CCc1c(F)ccc2cc(O)cc(N3CCc4c(nc(OC[C@@]56CCCN5C[C@H](F)C6)nc4N4CCCn5nc6c(c5C4)CCCN6)C3)c12 ; CCc1c(F)ccc2cc(O)cc(N3CCc4c(nc(OC[C@@]56CCCN5C[C@H](F)C6)nc4N4CCCn5nc(NC)c(OC)c5C4)C3)c12]

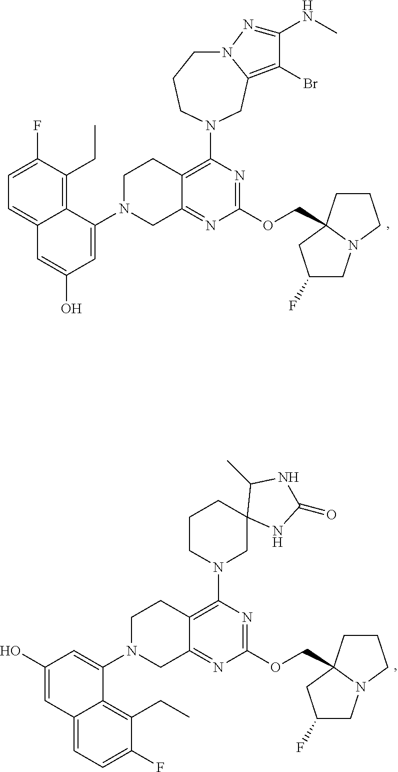 [SMILES: CCc1c(F)ccc2cc(O)cc(N3CCc4c(nc(OC[C@@]56CCCN5C[C@H](F)C6)nc4N4CCCn5nc(NC)c(Br)c5C4)C3)c12 ; CCc1c(F)ccc2cc(O)cc(N3CCc4c(nc(OC[C@@]56CCCN5C[C@H](F)C6)nc4N4CCCC5(C4)NC(=O)NC5C)C3)c12]

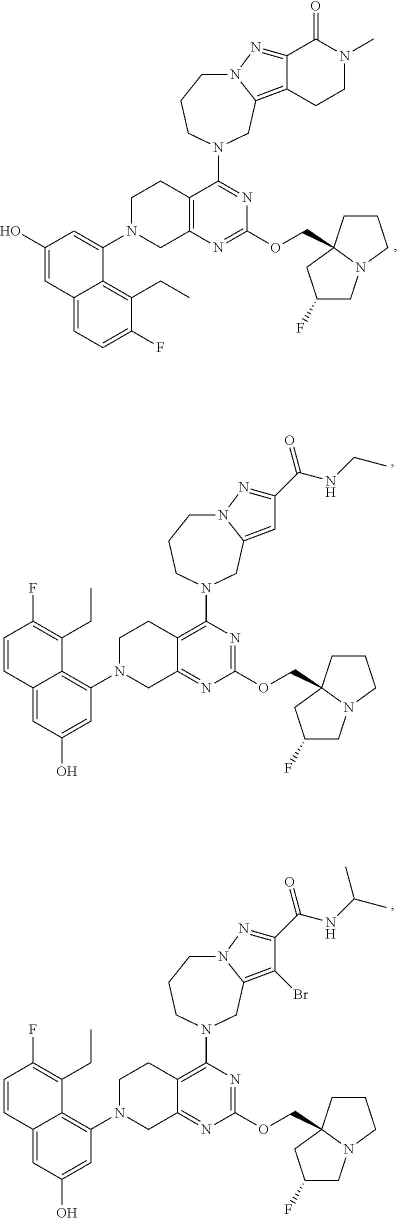 [SMILES: CCc1c(F)ccc2cc(O)cc(N3CCc4c(nc(OC[C@@]56CCCN5C[C@H](F)C6)nc4N4CCCn5nc6c(c5C4)CCN(C)C6=O)C3)c12 ; CCNC(=O)c1cc2n(n1)CCCN(c1nc(OC[C@@]34CCCN3C[C@H](F)C4)nc3c1CCN(c1cc(O)cc4ccc(F)c(CC)c14)C3)C2 ; CCc1c(F)ccc2cc(O)cc(N3CCc4c(nc(OC[C@@]56CCCN5C[C@H](F)C6)nc4N4CCCn5nc(C(=O)NC(C)C)c(Br)c5C4)C3)c12]

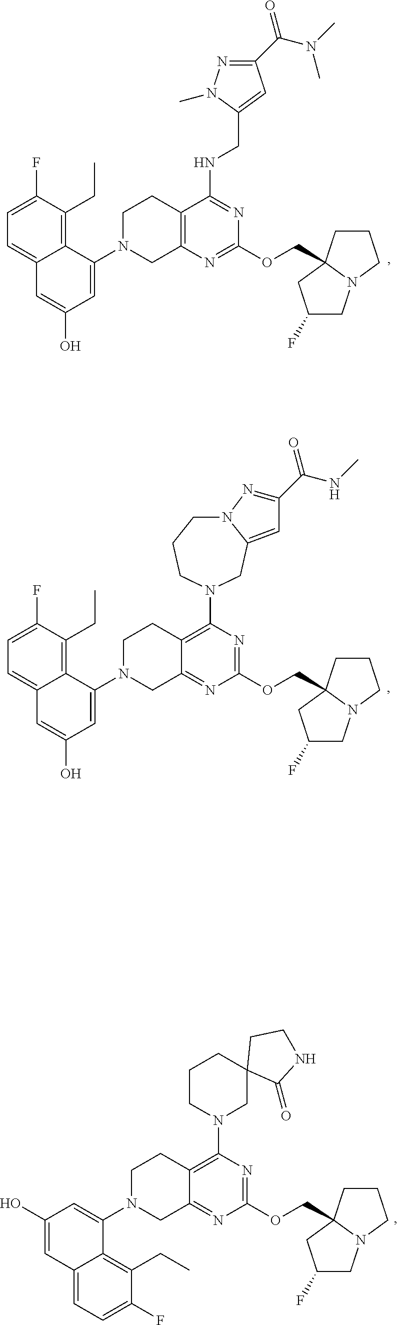 [SMILES: CCc1c(F)ccc2cc(O)cc(N3CCc4c(nc(OC[C@@]56CCCN5C[C@H](F)C6)nc4NCc4cc(C(=O)N(C)C)nn4C)C3)c12 ; CCc1c(F)ccc2cc(O)cc(N3CCc4c(nc(OC[C@@]56CCCN5C[C@H](F)C6)nc4N4CCCn5nc(C(=O)NC)cc5C4)C3)c12 ; CCc1c(F)ccc2cc(O)cc(N3CCc4c(nc(OC[C@@]56CCCN5C[C@H](F)C6)nc4N4CCCC5(CCNC5=O)C4)C3)c12]

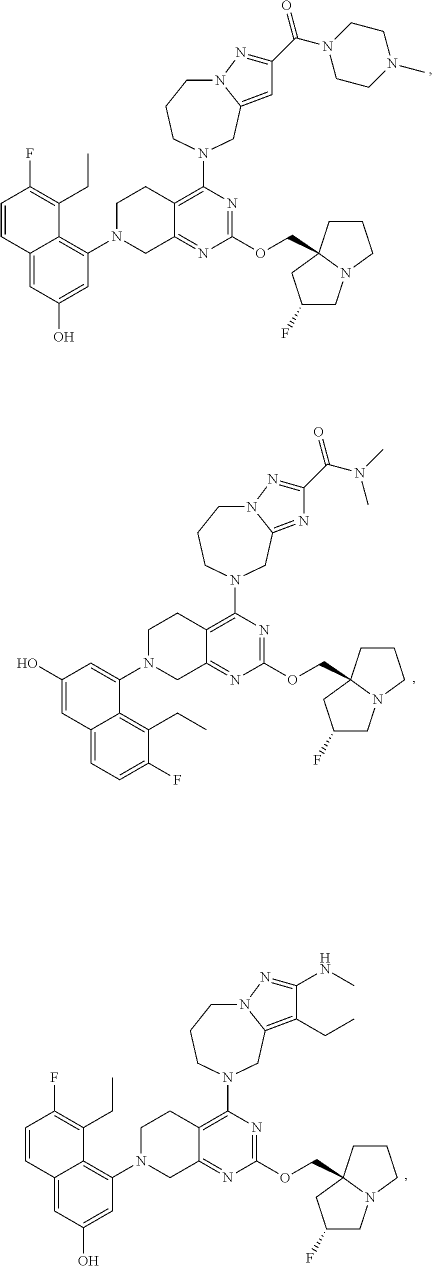 [SMILES: CCc1c(F)ccc2cc(O)cc(N3CCc4c(nc(OC[C@@]56CCCN5C[C@H](F)C6)nc4N4CCCn5nc(C(=O)N6CCN(C)CC6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N(C)C)nc5C4)C3)c12 ; CCc1c(NC)nn2c1CN(c1nc(OC[C@@]34CCCN3C[C@H](F)C4)nc3c1CCN(c1cc(O)cc4ccc(F)c(CC)c14)C3)CCC2]

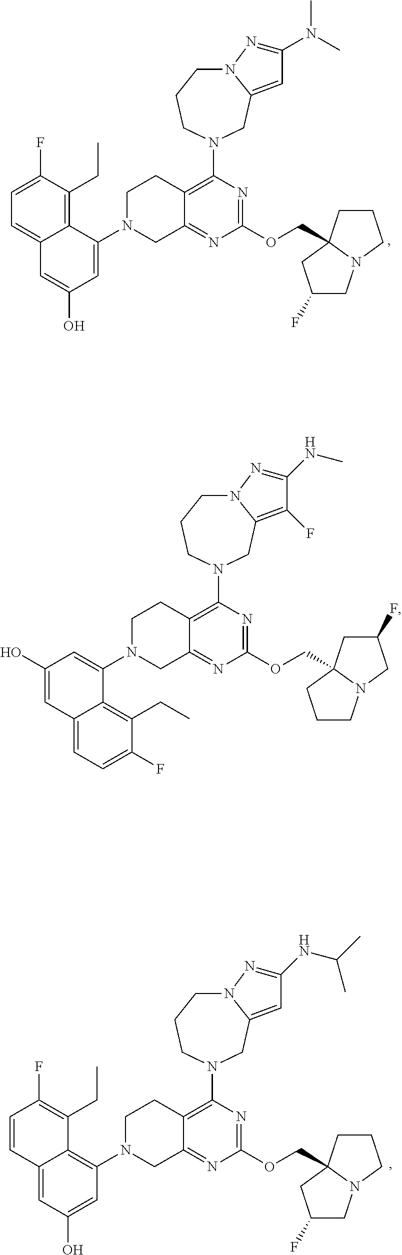 [SMILES: CCc1c(F)ccc2cc(O)cc(N3CCc4c(nc(OC[C@@]56CCCN5C[C@H](F)C6)nc4N4CCCn5nc(N(C)C)cc5C4)C3)c12 ; CCc1c(F)ccc2cc(O)cc(N3CCc4c(nc(OC[C@@]56CCCN5C[C@H](F)C6)nc4N4CCCn5nc(NC)c(F)c5C4)C3)c12 ; CCc1c(F)ccc2cc(O)cc(N3CCc4c(nc(OC[C@@]56CCCN5C[C@H](F)C6)nc4N4CCCn5nc(NC(C)C)cc5C4)C3)c12]

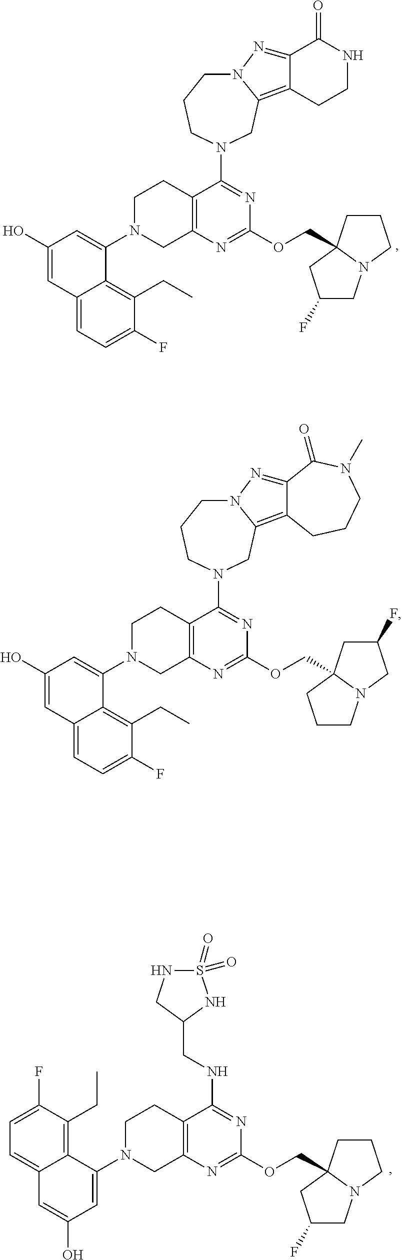 [SMILES: CCc1c(F)ccc2cc(O)cc(N3CCc4c(nc(OC[C@@]56CCCN5C[C@H](F)C6)nc4N4CCCn5nc6c(c5C4)CCNC6=O)C3)c12 ; CCc1c(F)ccc2cc(O)cc(N3CCc4c(nc(OC[C@@]56CCCN5C[C@H](F)C6)nc4N4CCCn5nc6c(c5C4)CCCN(C)C6=O)C3)c12 ; CCc1c(F)ccc2cc(O)cc(N3CCc4c(nc(OC[C@@]56CCCN5C[C@H](F)C6)nc4NCC4CNS(=O)(=O)N4)C3)c12]

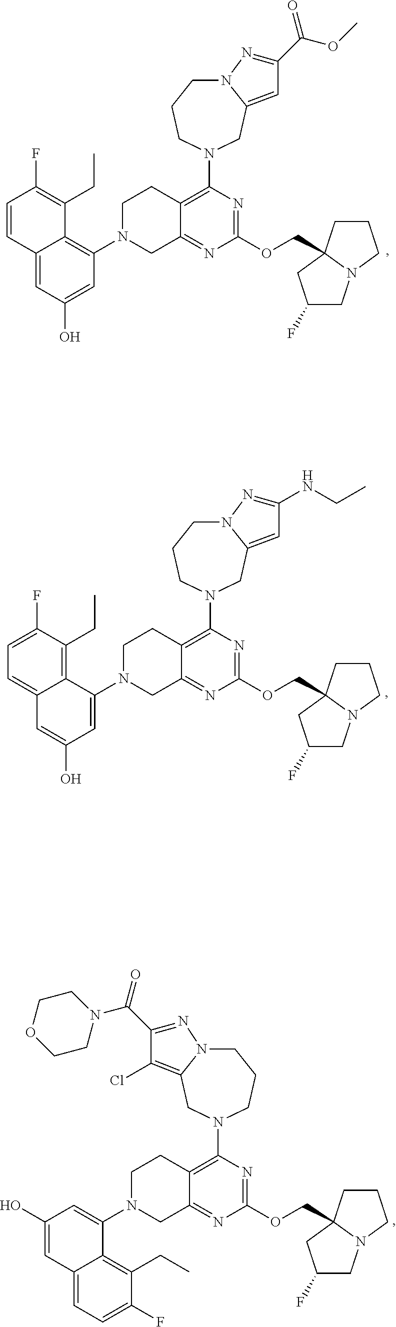 [SMILES: CCc1c(F)ccc2cc(O)cc(N3CCc4c(nc(OC[C@@]56CCCN5C[C@H](F)C6)nc4N4CCCn5nc(C(=O)OC)cc5C4)C3)c12 ; CCNc1cc2n(n1)CCCN(c1nc(OC[C@@]34CCCN3C[C@H](F)C4)nc3c1CCN(c1cc(O)cc4ccc(F)c(CC)c14)C3)C2 ; CCc1c(F)ccc2cc(O)cc(N3CCc4c(nc(OC[C@@]56CCCN5C[C@H](F)C6)nc4N4CCCn5nc(C(=O)N6CCOCC6)c(Cl)c5C4)C3)c12]

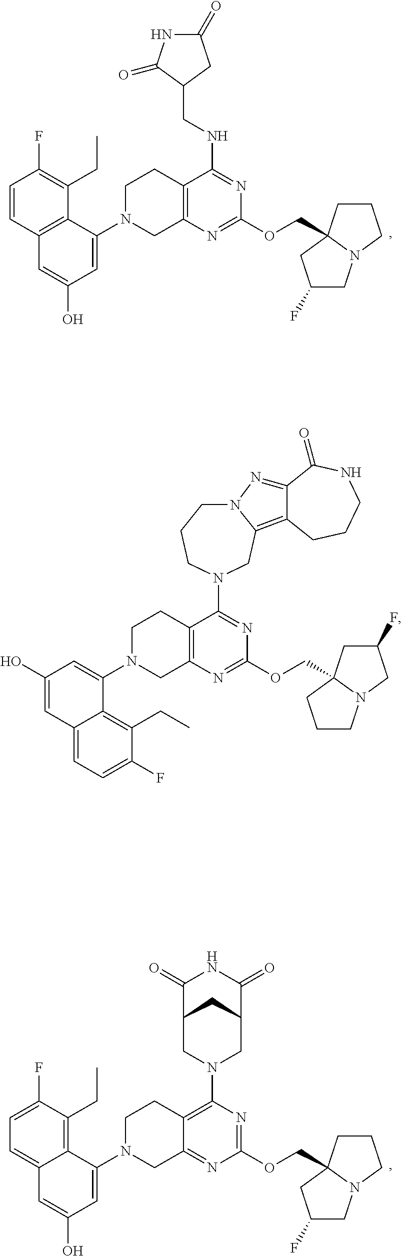 [SMILES: CCc1c(F)ccc2cc(O)cc(N3CCc4c(nc(OC[C@@]56CCCN5C[C@H](F)C6)nc4NCC4CC(=O)NC4=O)C3)c12 ; CCc1c(F)ccc2cc(O)cc(N3CCc4c(nc(OC[C@@]56CCCN5C[C@H](F)C6)nc4N4CCCn5nc6c(c5C4)CCCNC6=O)C3)c12 ; CCc1c(F)ccc2cc(O)cc(N3CCc4c(nc(OC[C@@]56CCCN5C[C@H](F)C6)nc4N4C[C@H]5C[C@@H](C4)C(=O)NC5=O)C3)c12]

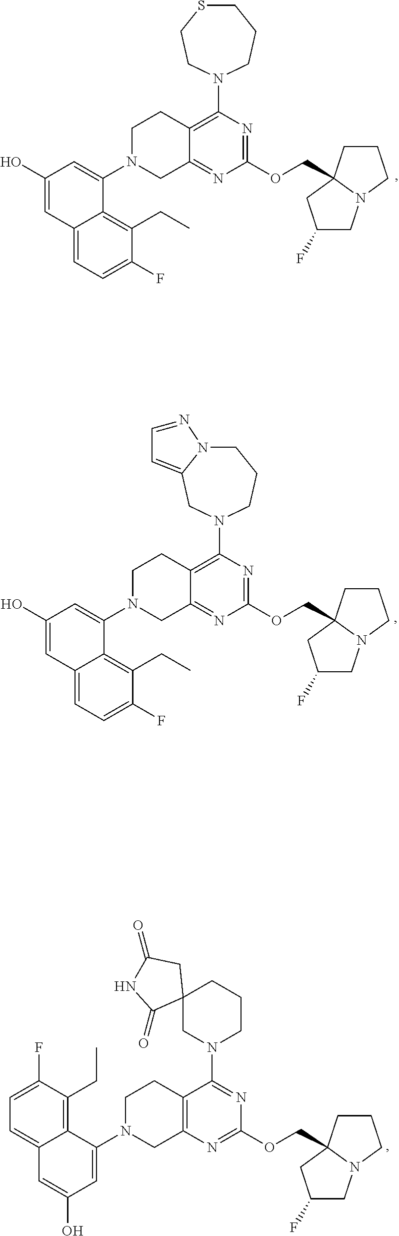 [SMILES: CCc1c(F)ccc2cc(O)cc(N3CCc4c(nc(OC[C@@]56CCCN5C[C@H](F)C6)nc4N4CCCSCC4)C3)c12 ; CCc1c(F)ccc2cc(O)cc(N3CCc4c(nc(OC[C@@]56CCCN5C[C@H](F)C6)nc4N4CCCn5nccc5C4)C3)c12 ; CCc1c(F)ccc2cc(O)cc(N3CCc4c(nc(OC[C@@]56CCCN5C[C@H](F)C6)nc4N4CCCC5(CC(=O)NC5=O)C4)C3)c12]

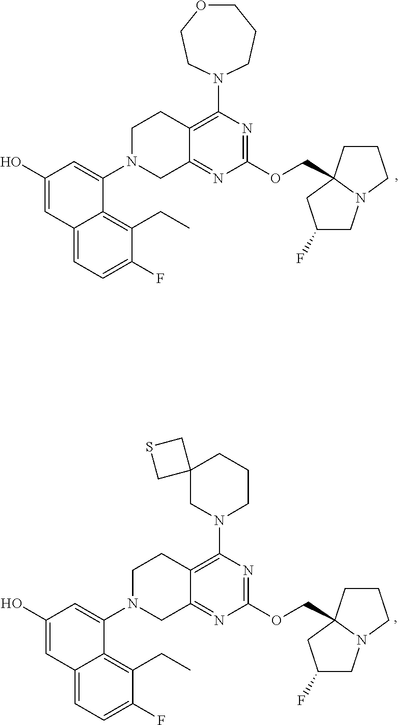 [SMILES: CCc1c(F)ccc2cc(O)cc(N3CCc4c(nc(OC[C@@]56CCCN5C[C@H](F)C6)nc4N4CCCOCC4)C3)c12 ; CCc1c(F)ccc2cc(O)cc(N3CCc4c(nc(OC[C@@]56CCCN5C[C@H](F)C6)nc4N4CCCC5(CSC5)C4)C3)c12]

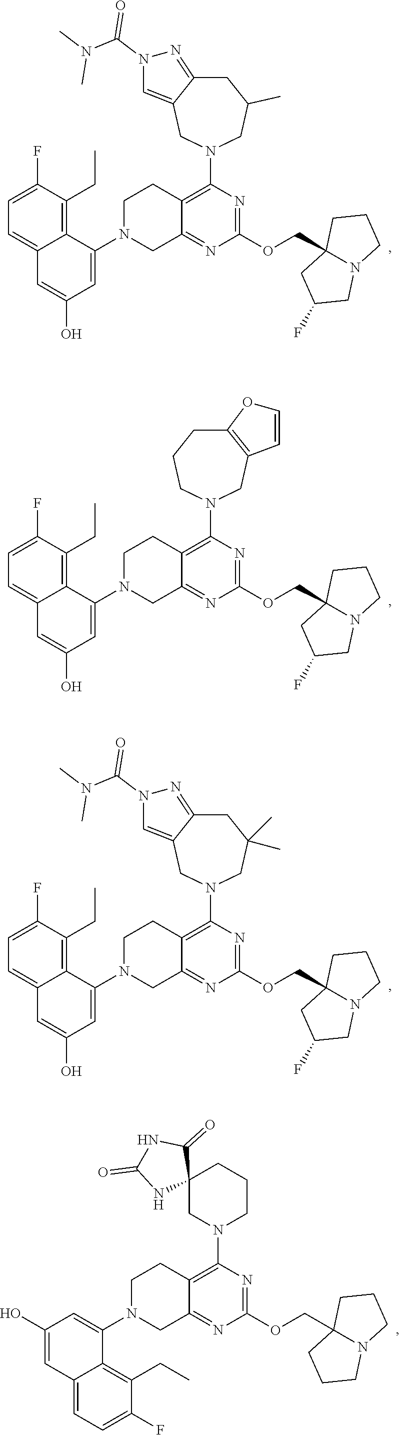 [SMILES: CCc1c(F)ccc2cc(O)cc(N3CCc4c(nc(OC[C@@]56CCCN5C[C@H](F)C6)nc4N4Cc5cn(C(=O)N(C)C)nc5CC(C)C4)C3)c12 ; CCc1c(F)ccc2cc(O)cc(N3CCc4c(nc(OC[C@@]56CCCN5C[C@H](F)C6)nc4N4CCCc5occc5C4)C3)c12 ; CCc1c(F)ccc2cc(O)cc(N3CCc4c(nc(OC[C@@]56CCCN5C[C@H](F)C6)nc4N4Cc5cn(C(=O)N(C)C)nc5CC(C)(C)C4)C3)c12 ; CCc1c(F)ccc2cc(O)cc(N3CCc4c(nc(OCC56CCCN5CCC6)nc4N4CCC[C@]5(C4)NC(=O)NC5=O)C3)c12]

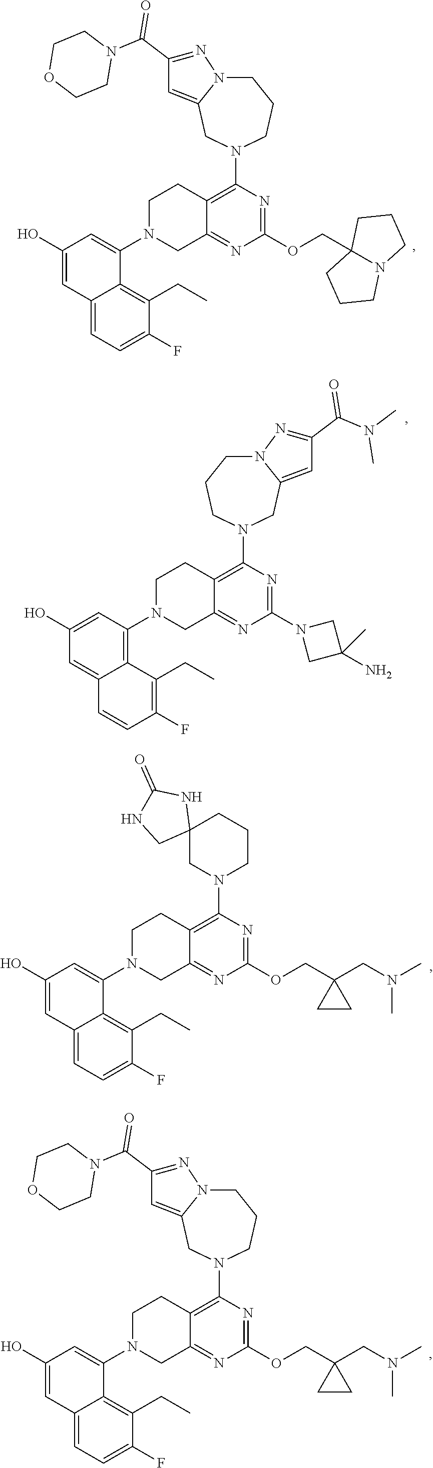

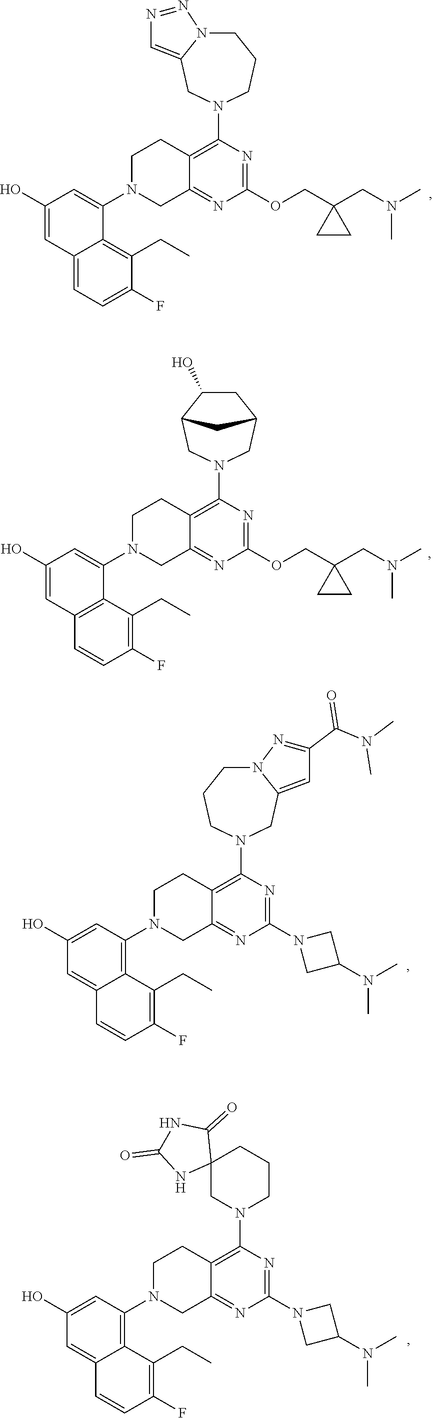 [SMILES: CCc1c(F)ccc2cc(O)cc(N3CCc4c(nc(OCC5(CN(C)C)CC5)nc4N4CCCn5nncc5C4)C3)c12 ; CCc1c(F)ccc2cc(O)cc(N3CCc4c(nc(OCC5(CN(C)C)CC5)nc4N4C[C@@H]5C[C@H](C4)[C@H](O)C5)C3)c12 ; CCc1c(F)ccc2cc(O)cc(N3CCc4c(nc(N5CC(N(C)C)C5)nc4N4CCCn5nc(C(=O)N(C)C)cc5C4)C3)c12 ; CCc1c(F)ccc2cc(O)cc(N3CCc4c(nc(N5CC(N(C)C)C5)nc4N4CCCC5(C4)NC(=O)NC5=O)C3)c12]

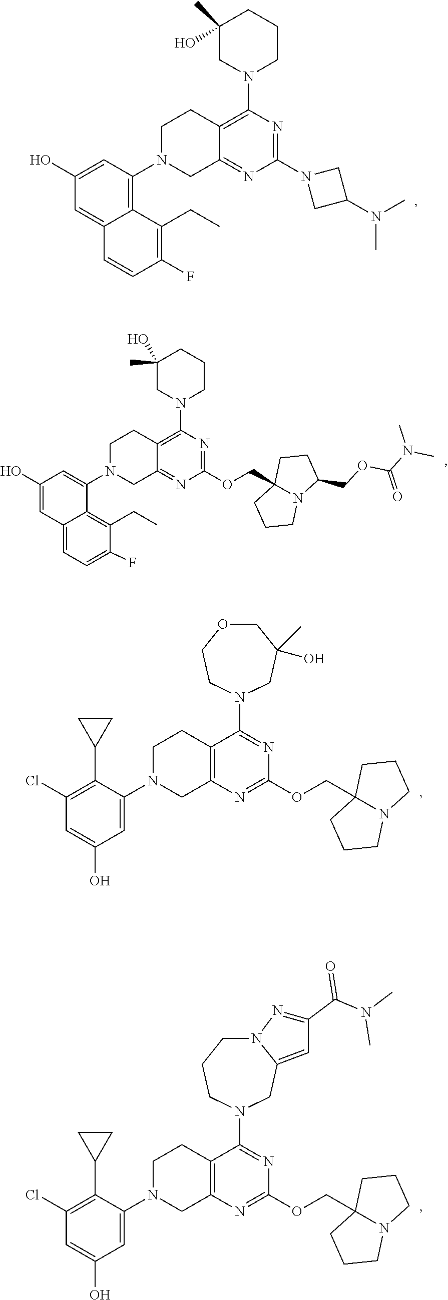 [SMILES: CCc1c(F)ccc2cc(O)cc(N3CCc4c(nc(N5CC(N(C)C)C5)nc4N4CCC[C@@](C)(O)C4)C3)c12 ; CCc1c(F)ccc2cc(O)cc(N3CCc4c(nc(OC[C@]56CCCN5[C@H](COC(=O)N(C)C)CC6)nc4N4CCC[C@@](C)(O)C4)C3)c12 ; CC1(O)COCCN(c2nc(OCC34CCCN3CCC4)nc3c2CCN(c2cc(O)cc(Cl)c2C2CC2)C3)C1 ; CN(C)C(=O)c1cc2n(n1)CCCN(c1nc(OCC34CCCN3CCC4)nc3c1CCN(c1cc(O)cc(Cl)c1C1CC1)C3)C2]

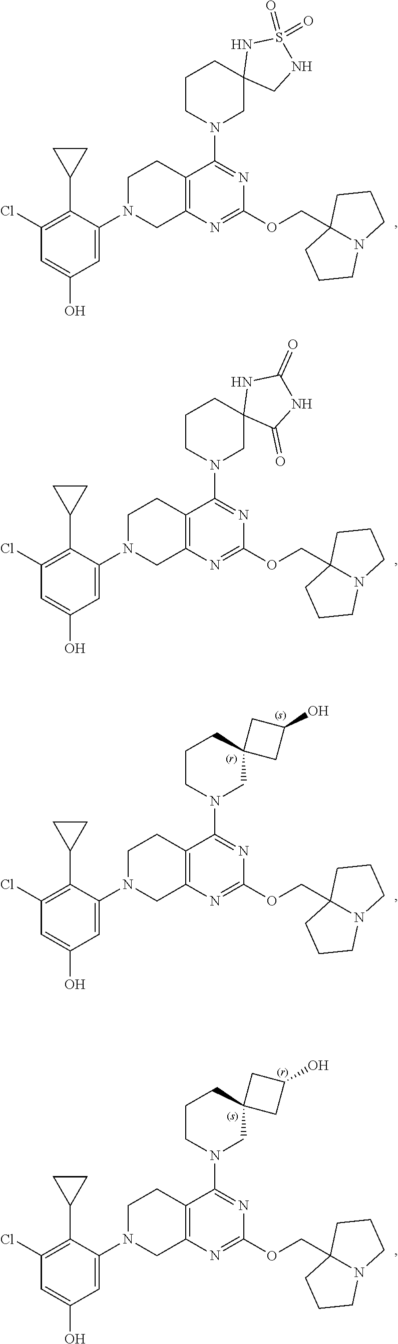 [SMILES: O=S1(=O)NCC2(CCCN(c3nc(OCC45CCCN4CCC5)nc4c3CCN(c3cc(O)cc(Cl)c3C3CC3)C4)C2)N1 ; O=C1NC(=O)C2(CCCN(c3nc(OCC45CCCN4CCC5)nc4c3CCN(c3cc(O)cc(Cl)c3C3CC3)C4)C2)N1 ; Oc1cc(Cl)c(C2CC2)c(N2CCc3c(nc(OCC45CCCN4CCC5)nc3N3CCC[C@]4(C3)C[C@H](O)C4)C2)c1 ; Oc1cc(Cl)c(C2CC2)c(N2CCc3c(nc(OCC45CCCN4CCC5)nc3N3CCC[C@]4(C3)C[C@@H](O)C4)C2)c1]

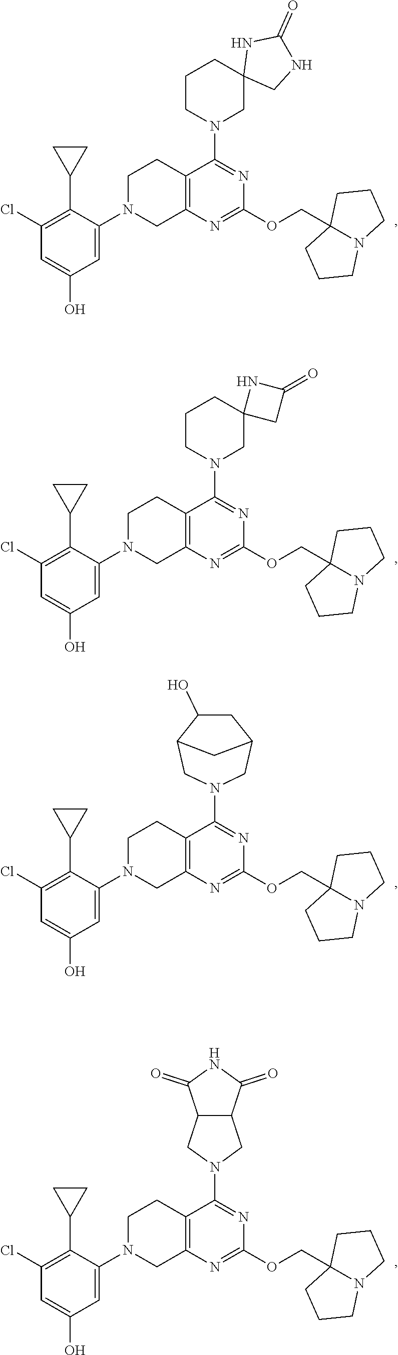 [SMILES: O=C1NCC2(CCCN(c3nc(OCC45CCCN4CCC5)nc4c3CCN(c3cc(O)cc(Cl)c3C3CC3)C4)C2)N1 ; O=C1CC2(CCCN(c3nc(OCC45CCCN4CCC5)nc4c3CCN(c3cc(O)cc(Cl)c3C3CC3)C4)C2)N1 ; Oc1cc(Cl)c(C2CC2)c(N2CCc3c(nc(OCC45CCCN4CCC5)nc3N3CC4CC(O)C(C4)C3)C2)c1 ; O=C1NC(=O)C2CN(c3nc(OCC45CCCN4CCC5)nc4c3CCN(c3cc(O)cc(Cl)c3C3CC3)C4)CC12]

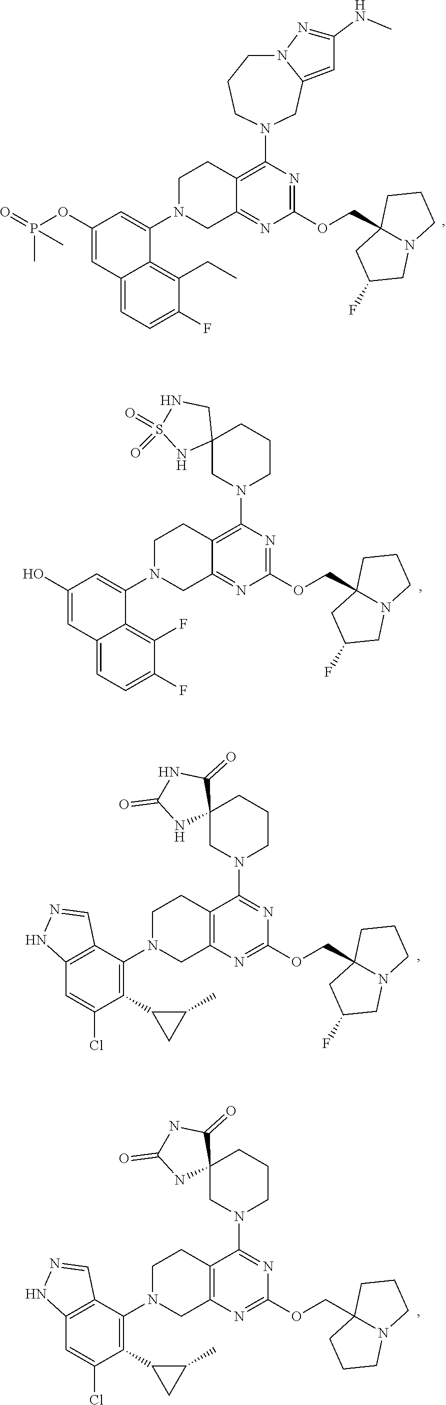

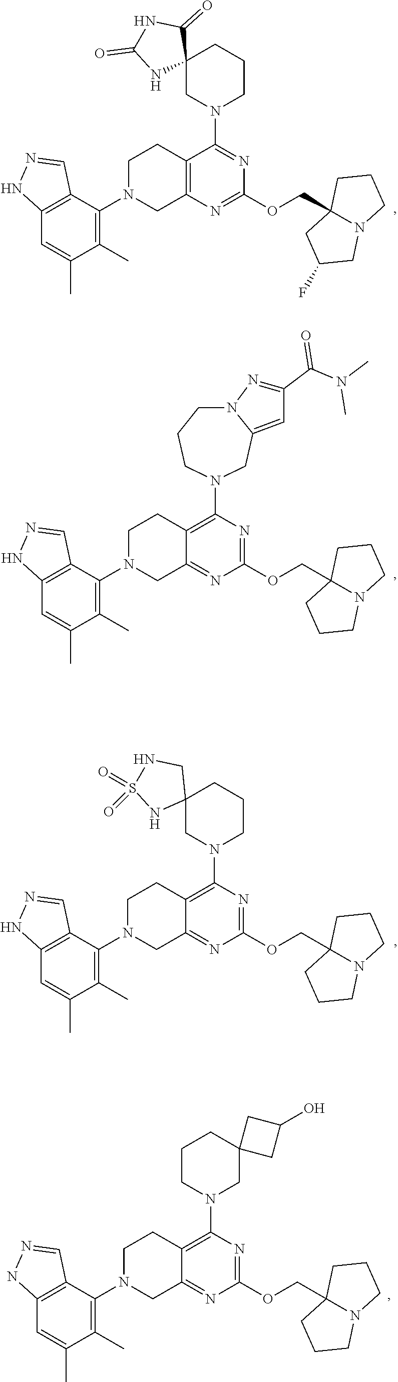

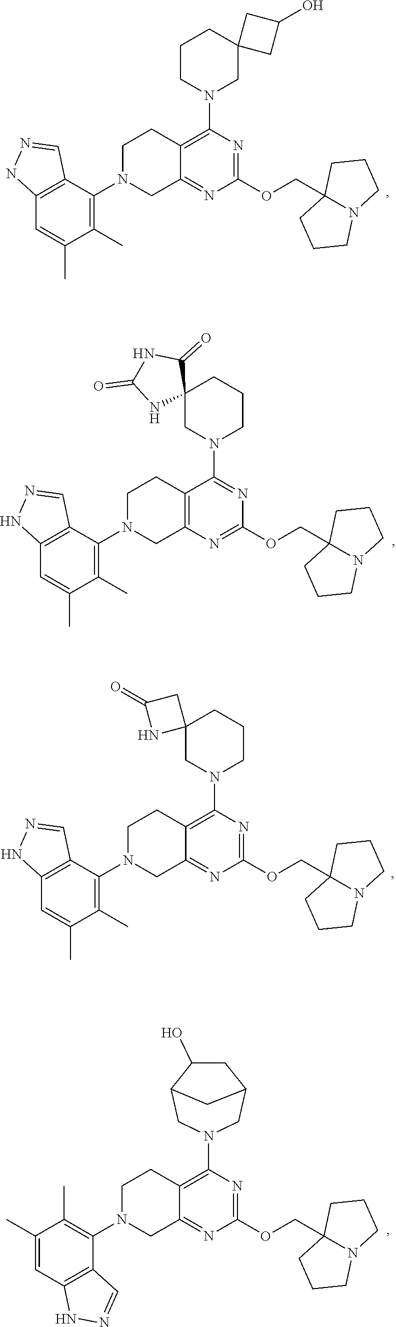 [SMILES: Cc1cc2[nH]ncc2c(N2CCc3c(nc(OCC45CCCN4CCC5)nc3N3CCCC4(CC(O)C4)C3)C2)c1C ; Cc1cc2[nH]ncc2c(N2CCc3c(nc(OCC45CCCN4CCC5)nc3N3CCC[C@]4(C3)NC(=O)NC4=O)C2)c1C ; Cc1cc2[nH]ncc2c(N2CCc3c(nc(OCC45CCCN4CCC5)nc3N3CCCC4(CC(=O)N4)C3)C2)c1C ; Cc1cc2[nH]ncc2c(N2CCc3c(nc(OCC45CCCN4CCC5)nc3N3CC4CC(O)C(C4)C3)C2)c1C]

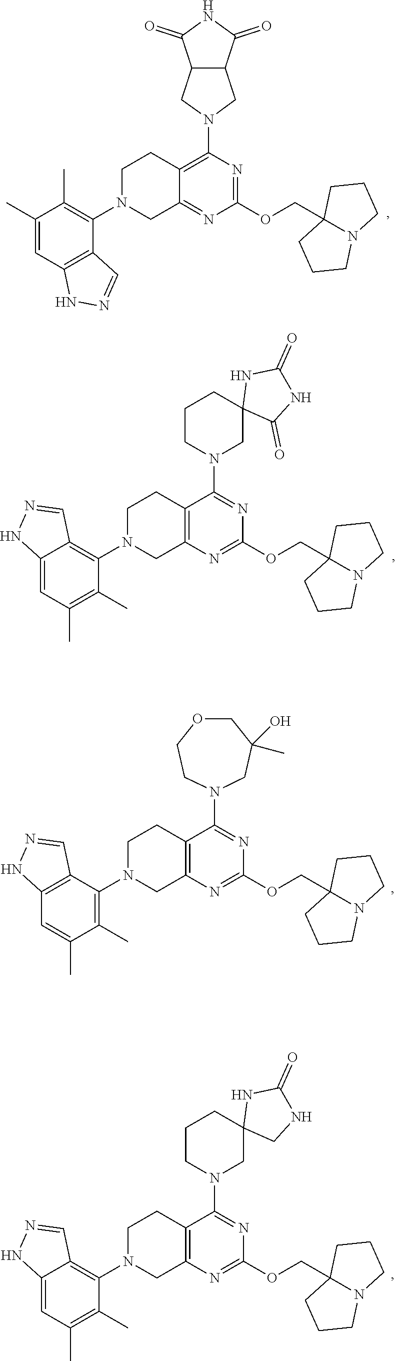 [SMILES: Cc1cc2[nH]ncc2c(N2CCc3c(nc(OCC45CCCN4CCC5)nc3N3CC4C(=O)NC(=O)C4C3)C2)c1C ; Cc1cc2[nH]ncc2c(N2CCc3c(nc(OCC45CCCN4CCC5)nc3N3CCCC4(C3)NC(=O)NC4=O)C2)c1C ; Cc1cc2[nH]ncc2c(N2CCc3c(nc(OCC45CCCN4CCC5)nc3N3CCOCC(C)(O)C3)C2)c1C ; Cc1cc2[nH]ncc2c(N2CCc3c(nc(OCC45CCCN4CCC5)nc3N3CCCC4(CNC(=O)N4)C3)C2)c1C]

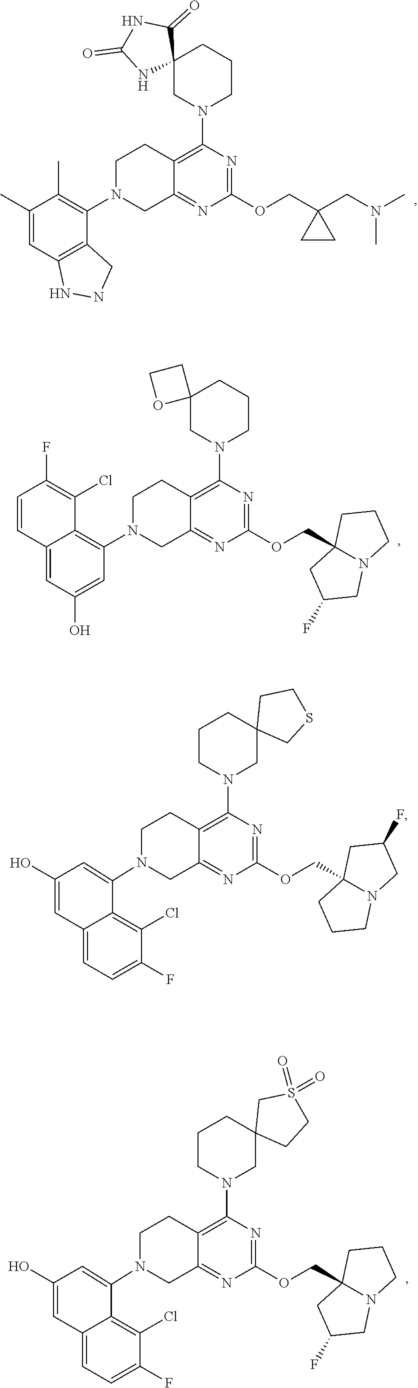

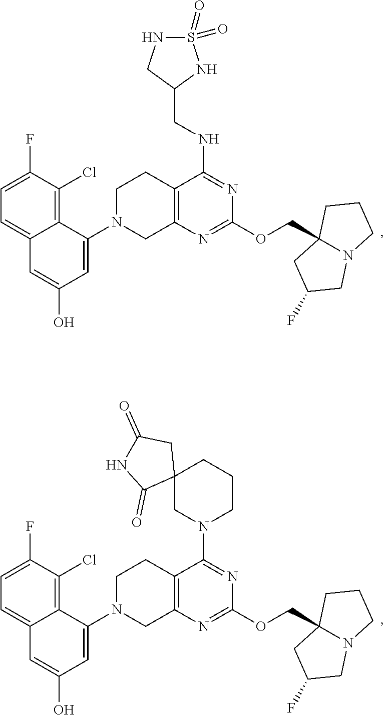 [SMILES: O=S1(=O)NCC(CNc2nc(OC[C@@]34CCCN3C[C@H](F)C4)nc3c2CCN(c2cc(O)cc4ccc(F)c(Cl)c24)C3)N1 ; O=C1CC2(CCCN(c3nc(OC[C@@]45CCCN4C[C@H](F)C5)nc4c3CCN(c3cc(O)cc5ccc(F)c(Cl)c35)C4)C2)C(=O)N1]

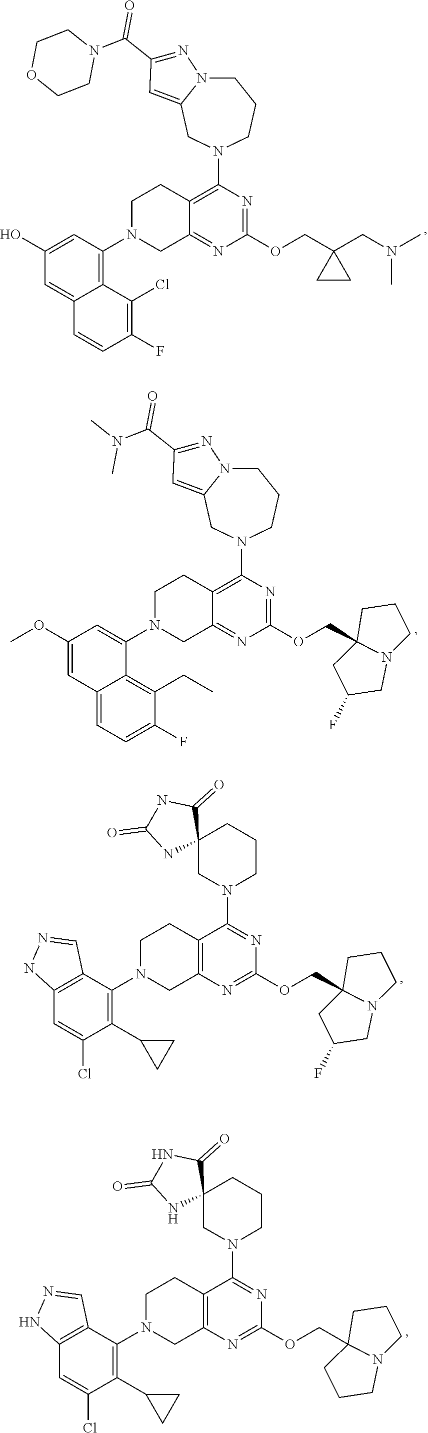

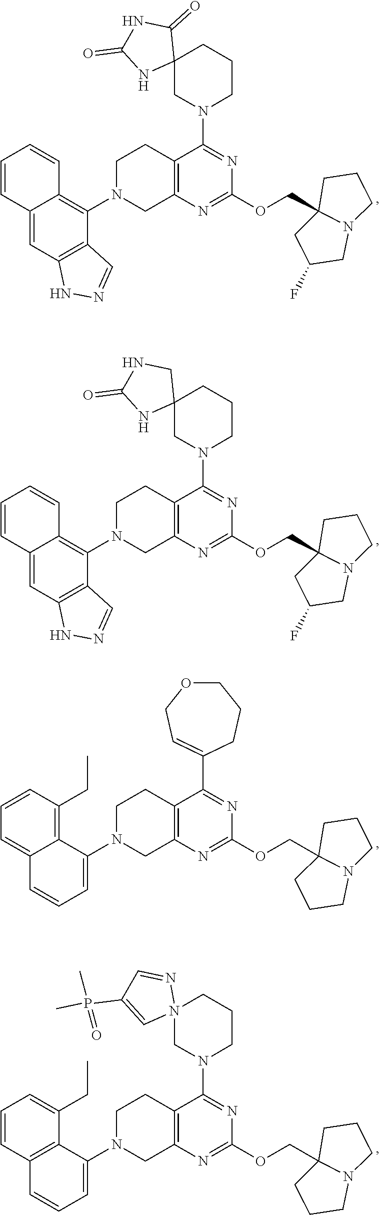

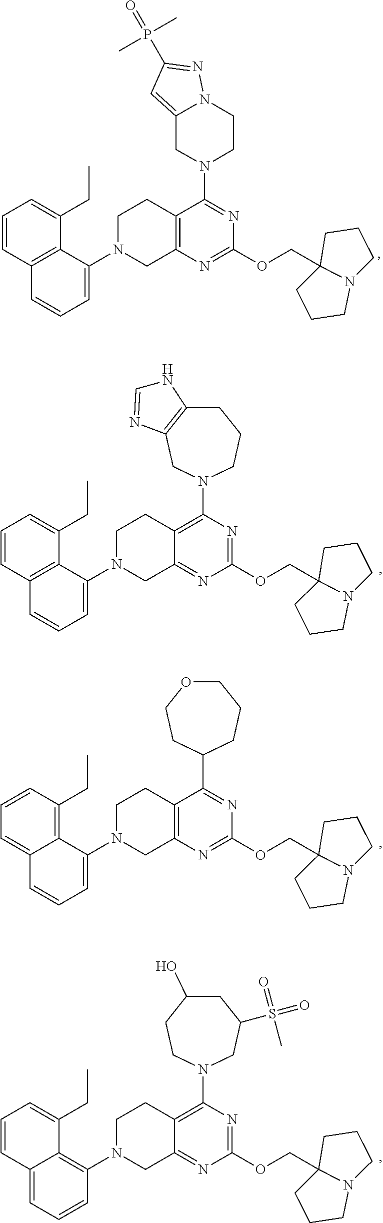 [SMILES: CCc1cccc2cccc(N3CCc4c(nc(OCC56CCCN5CCC6)nc4N4CCn5nc(P(C)(C)=O)cc5C4)C3)c12 ; CCc1cccc2cccc(N3CCc4c(nc(OCC56CCCN5CCC6)nc4N4CCCc5[nH]cnc5C4)C3)c12 ; CCc1cccc2cccc(N3CCc4c(nc(OCC56CCCN5CCC6)nc4C4CCCOCC4)C3)c12 ; CCc1cccc2cccc(N3CCc4c(nc(OCC56CCCN5CCC6)nc4N4CCC(O)CC(S(C)(=O)=O)C4)C3)c12]

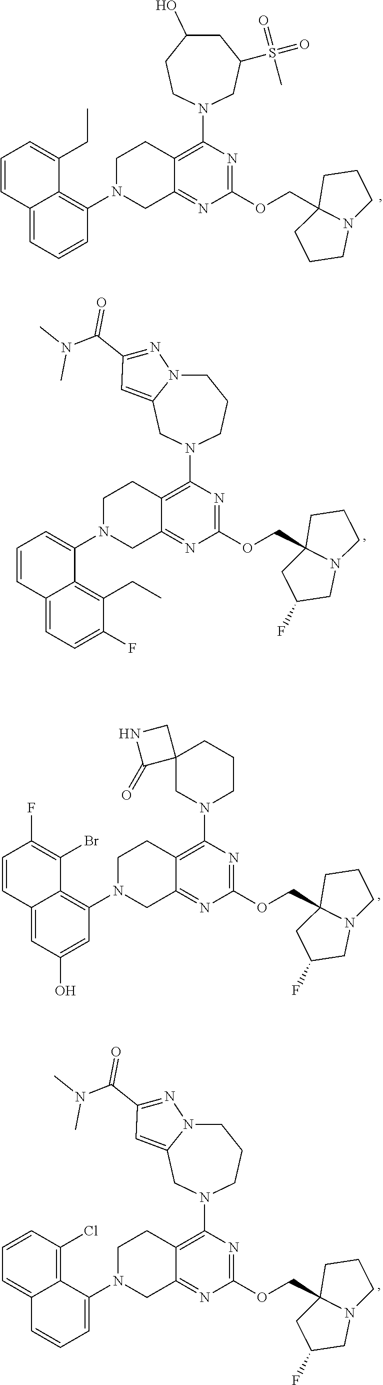 [SMILES: CCc1cccc2cccc(N3CCc4c(nc(OCC56CCCN5CCC6)nc4N4CCC(O)CC(S(C)(=O)=O)C4)C3)c12 ; CCc1c(F)ccc2cccc(N3CCc4c(nc(OC[C@@]56CCCN5C[C@H](F)C6)nc4N4CCCn5nc(C(=O)N(C)C)cc5C4)C3)c12 ; O=C1NCC12CCCN(c1nc(OC[C@@]34CCCN3C[C@H](F)C4)nc3c1CCN(c1cc(O)cc4ccc(F)c(Br)c14)C3)C2 ; CN(C)C(=O)c1cc2n(n1)CCCN(c1nc(OC[C@@]34CCCN3C[C@H](F)C4)nc3c1CCN(c1cccc4cccc(Cl)c14)C3)C2]

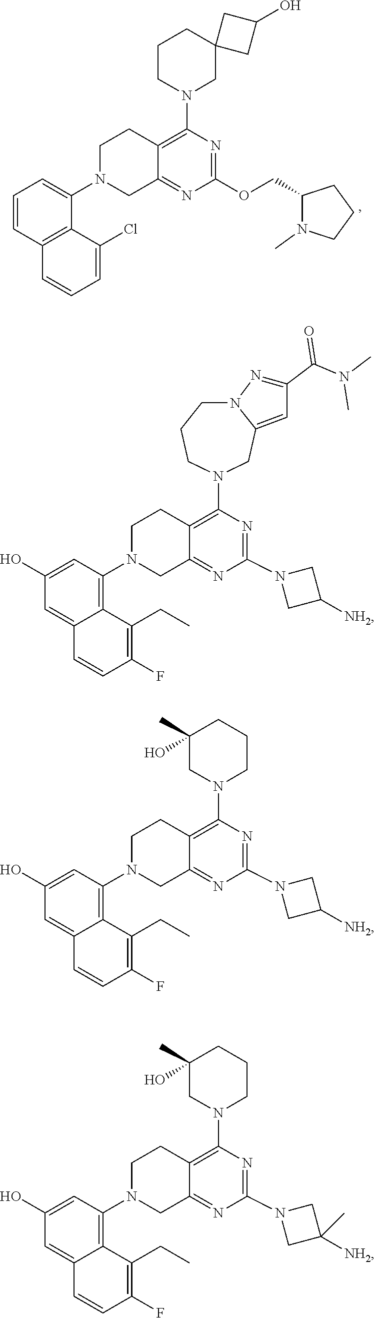

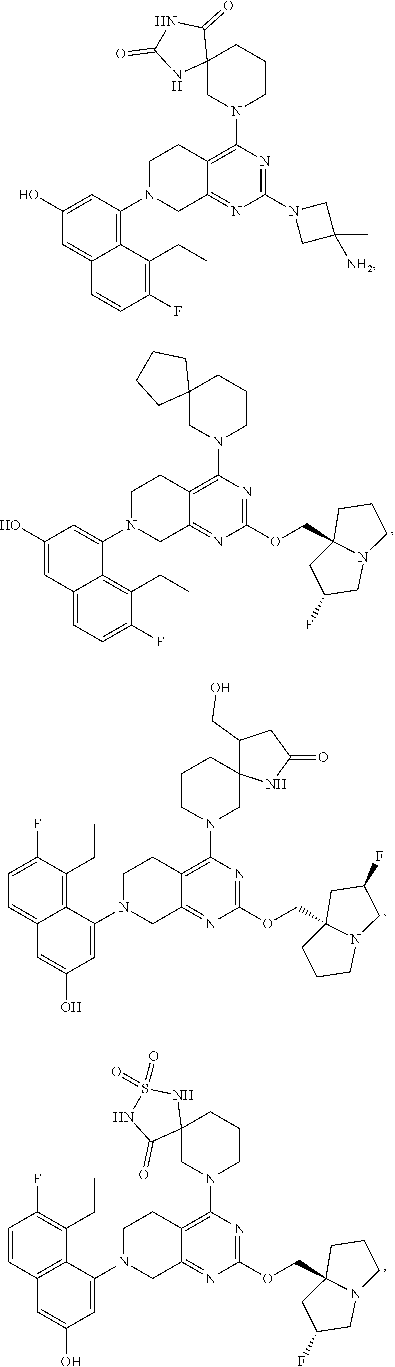 [SMILES: CCc1c(F)ccc2cc(O)cc(N3CCc4c(nc(N5CC(C)(N)C5)nc4N4CCCC5(C4)NC(=O)NC5=O)C3)c12 ; CCc1c(F)ccc2cc(O)cc(N3CCc4c(nc(OC[C@@]56CCCN5C[C@H](F)C6)nc4N4CCCC5(CCCC5)C4)C3)c12 ; CCc1c(F)ccc2cc(O)cc(N3CCc4c(nc(OC[C@@]56CCCN5C[C@H](F)C6)nc4N4CCCC5(C4)NC(=O)CC5CO)C3)c12 ; CCc1c(F)ccc2cc(O)cc(N3CCc4c(nc(OC[C@@]56CCCN5C[C@H](F)C6)nc4N4CCCC5(C4)NS(=O)(=O)NC5=O)C3)c12]

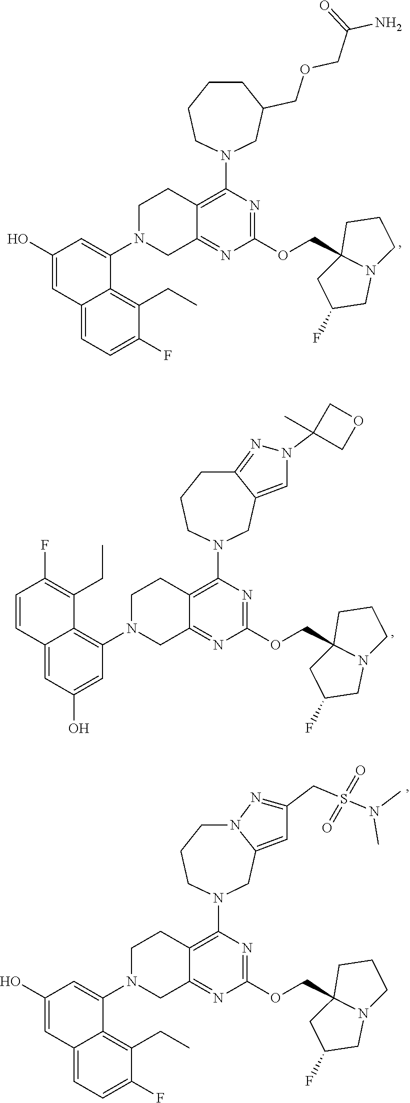 [SMILES: CCc1c(F)ccc2cc(O)cc(N3CCc4c(nc(OC[C@@]56CCCN5C[C@H](F)C6)nc4N4CCCCC(COCC(N)=O)C4)C3)c12 ; CCc1c(F)ccc2cc(O)cc(N3CCc4c(nc(OC[C@@]56CCCN5C[C@H](F)C6)nc4N4CCCc5nn(C6(C)COC6)cc5C4)C3)c12 ; CCc1c(F)ccc2cc(O)cc(N3CCc4c(nc(OC[C@@]56CCCN5C[C@H](F)C6)nc4N4CCCn5nc(CS(=O)(=O)N(C)C)cc5C4)C3)c12]

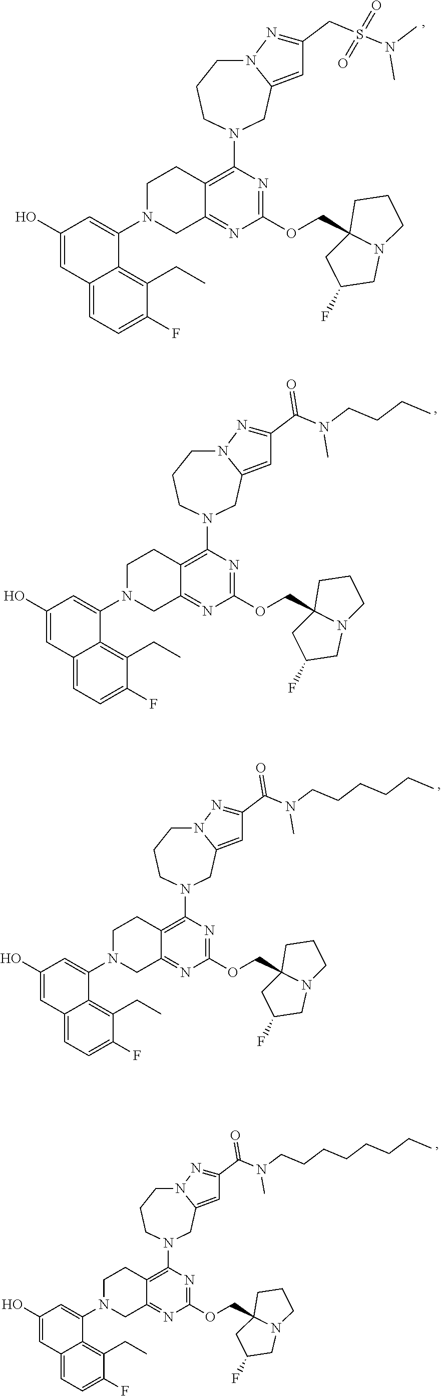 [SMILES: CCc1c(F)ccc2cc(O)cc(N3CCc4c(nc(OC[C@@]56CCCN5C[C@H](F)C6)nc4N4CCCn5nc(CS(=O)(=O)N(C)C)cc5C4)C3)c12 ; CCCCN(C)C(=O)c1cc2n(n1)CCCN(c1nc(OC[C@@]34CCCN3C[C@H](F)C4)nc3c1CCN(c1cc(O)cc4ccc(F)c(CC)c14)C3)C2 ; CCCCCCN(C)C(=O)c1cc2n(n1)CCCN(c1nc(OC[C@@]34CCCN3C[C@H](F)C4)nc3c1CCN(c1cc(O)cc4ccc(F)c(CC)c14)C3)C2 ; CCCCCCCCN(C)C(=O)c1cc2n(n1)CCCN(c1nc(OC[C@@]34CCCN3C[C@H](F)C4)nc3c1CCN(c1cc(O)cc4ccc(F)c(CC)c14)C3)C2]

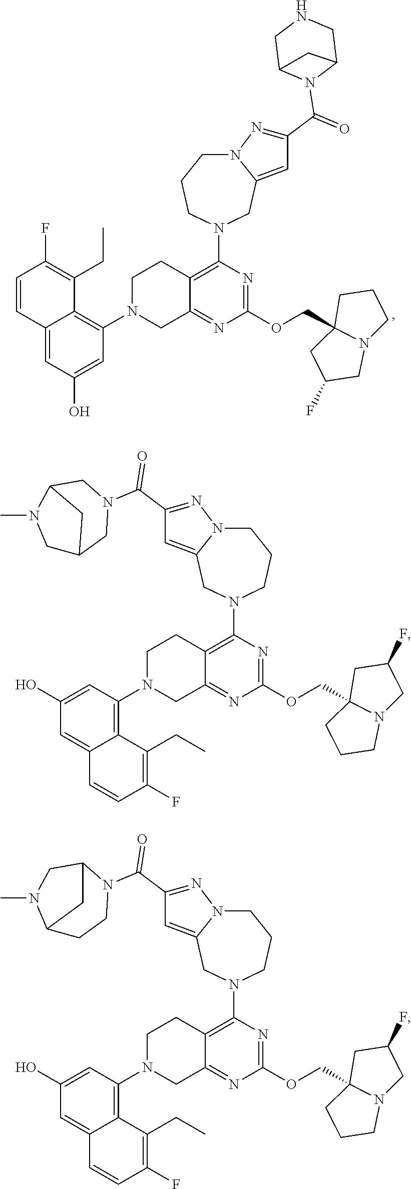

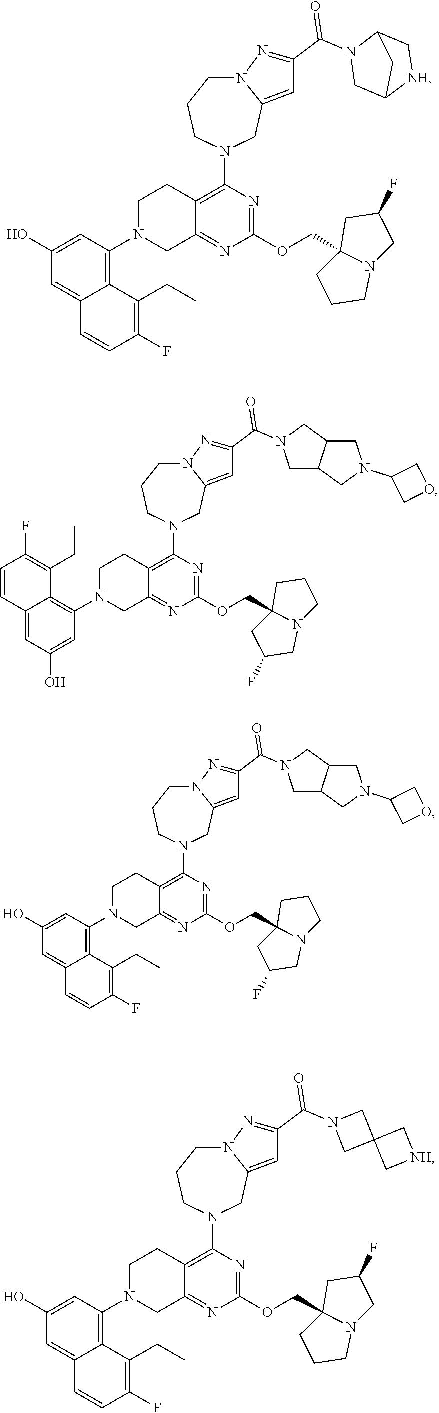 [SMILES: CCc1c(F)ccc2cc(O)cc(N3CCc4c(nc(OC[C@@]56CCCN5C[C@H](F)C6)nc4N4CCCn5nc(C(=O)N6CC7CC6CN7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CN(C8COC8)CC7C6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CN(C8COC8)CC7C6)cc5C4)C3)c12 ; CCc1c(F)ccc2cc(O)cc(N3CCc4c(nc(OC[C@]56CCCN5C[C@H](F)C6)nc4N4CCCn5nc(C(=O)N6CC7(CNC7)C6)cc5C4)C3)c12]

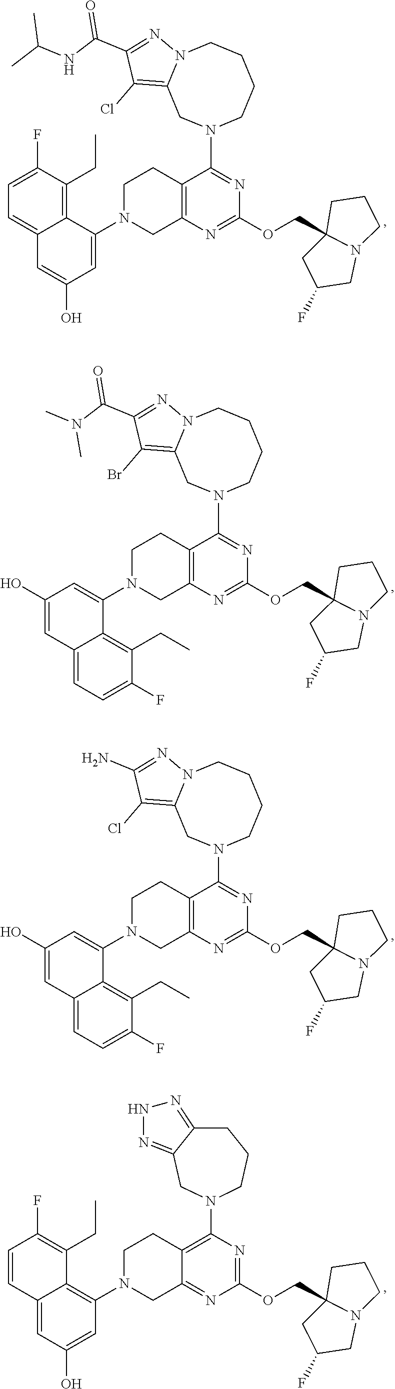 [SMILES: CCc1c(F)ccc2cc(O)cc(N3CCc4c(nc(OC[C@@]56CCCN5C[C@H](F)C6)nc4N4CCCCn5nc(C(=O)NC(C)C)c(Cl)c5C4)C3)c12 ; CCc1c(F)ccc2cc(O)cc(N3CCc4c(nc(OC[C@@]56CCCN5C[C@H](F)C6)nc4N4CCCCn5nc(C(=O)N(C)C)c(Br)c5C4)C3)c12 ; CCc1c(F)ccc2cc(O)cc(N3CCc4c(nc(OC[C@@]56CCCN5C[C@H](F)C6)nc4N4CCCCn5nc(N)c(Cl)c5C4)C3)c12 ; CCc1c(F)ccc2cc(O)cc(N3CCc4c(nc(OC[C@@]56CCCN5C[C@H](F)C6)nc4N4CCCc5n[nH]nc5C4)C3)c12]

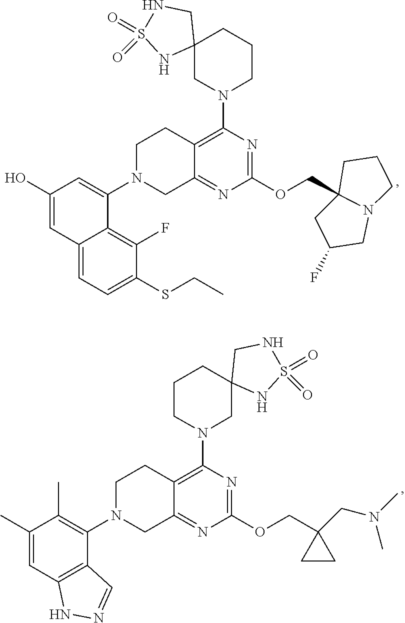

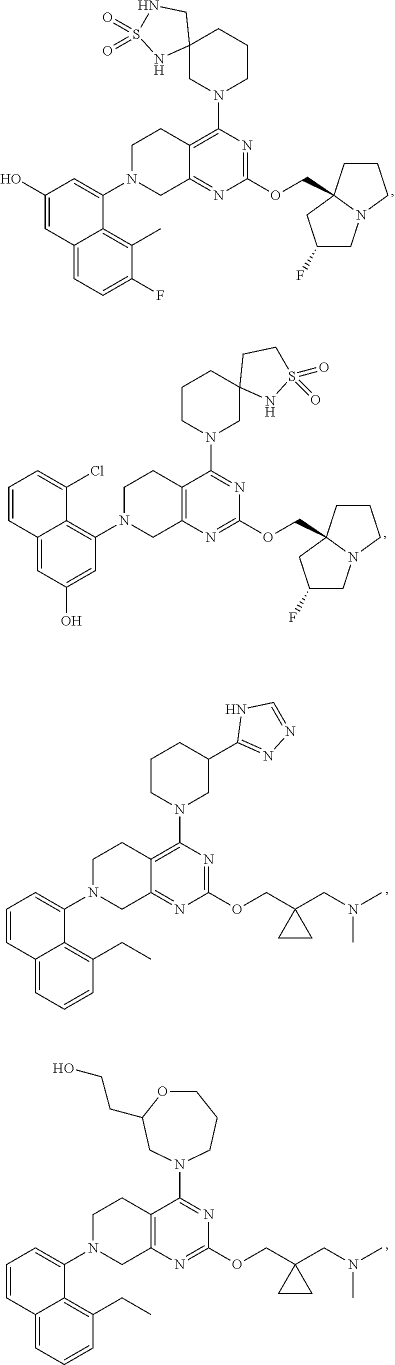 [SMILES: Cc1c(F)ccc2cc(O)cc(N3CCc4c(nc(OC[C@@]56CCCN5C[C@H](F)C6)nc4N4CCCC5(CNS(=O)(=O)N5)C4)C3)c12 ; O=S1(=O)CCC2(CCCN(c3nc(OC[C@@]45CCCN4C[C@H](F)C5)nc4c3CCN(c3cc(O)cc5cccc(Cl)c35)C4)C2)N1 ; CCc1cccc2cccc(N3CCc4c(nc(OCC5(CN(C)C)CC5)nc4N4CCCC(c5nnc[nH]5)C4)C3)c12 ; CCc1cccc2cccc(N3CCc4c(nc(OCC5(CN(C)C)CC5)nc4N4CCCOC(CCO)C4)C3)c12]

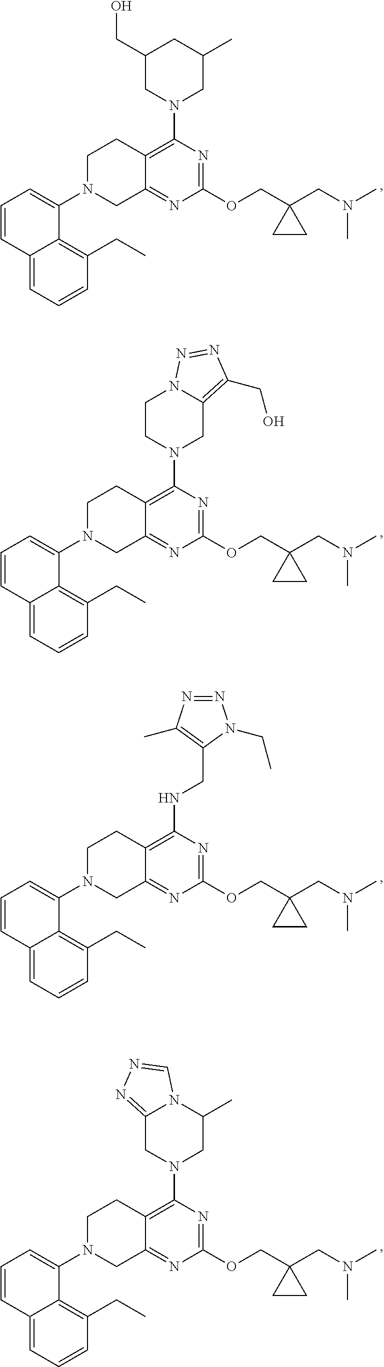 [SMILES: CCc1cccc2cccc(N3CCc4c(nc(OCC5(CN(C)C)CC5)nc4N4CC(C)CC(CO)C4)C3)c12 ; CCc1cccc2cccc(N3CCc4c(nc(OCC5(CN(C)C)CC5)nc4N4CCn5nnc(CO)c5C4)C3)c12 ; CCc1cccc2cccc(N3CCc4c(nc(OCC5(CN(C)C)CC5)nc4NCc4c(C)nnn4CC)C3)c12 ; CCc1cccc2cccc(N3CCc4c(nc(OCC5(CN(C)C)CC5)nc4N4Cc5nncn5C(C)C4)C3)c12]

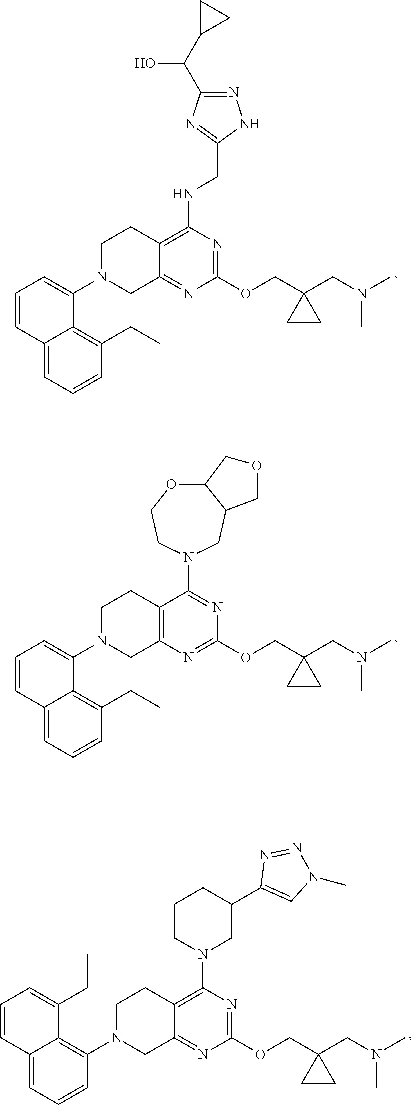 [SMILES: CCc1cccc2cccc(N3CCc4c(nc(OCC5(CN(C)C)CC5)nc4NCc4nc(C(O)C5CC5)n[nH]4)C3)c12 ; CCc1cccc2cccc(N3CCc4c(nc(OCC5(CN(C)C)CC5)nc4N4CCOC5COCC5C4)C3)c12 ; CCc1cccc2cccc(N3CCc4c(nc(OCC5(CN(C)C)CC5)nc4N4CCCC(c5cn(C)nn5)C4)C3)c12]

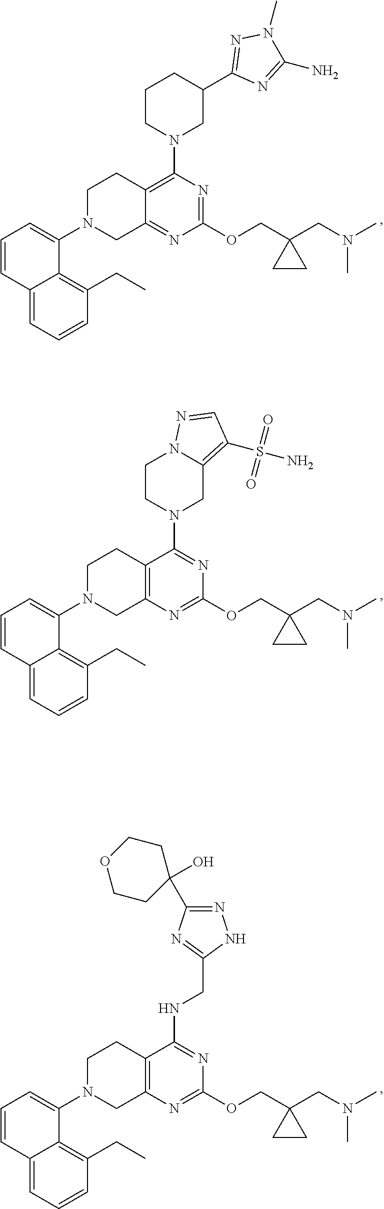 [SMILES: CCc1cccc2cccc(N3CCc4c(nc(OCC5(CN(C)C)CC5)nc4N4CCCC(c5nc(N)n(C)n5)C4)C3)c12 ; CCc1cccc2cccc(N3CCc4c(nc(OCC5(CN(C)C)CC5)nc4N4CCn5ncc(S(N)(=O)=O)c5C4)C3)c12 ; CCc1cccc2cccc(N3CCc4c(nc(OCC5(CN(C)C)CC5)nc4NCc4nc(C5(O)CCOCC5)n[nH]4)C3)c12]

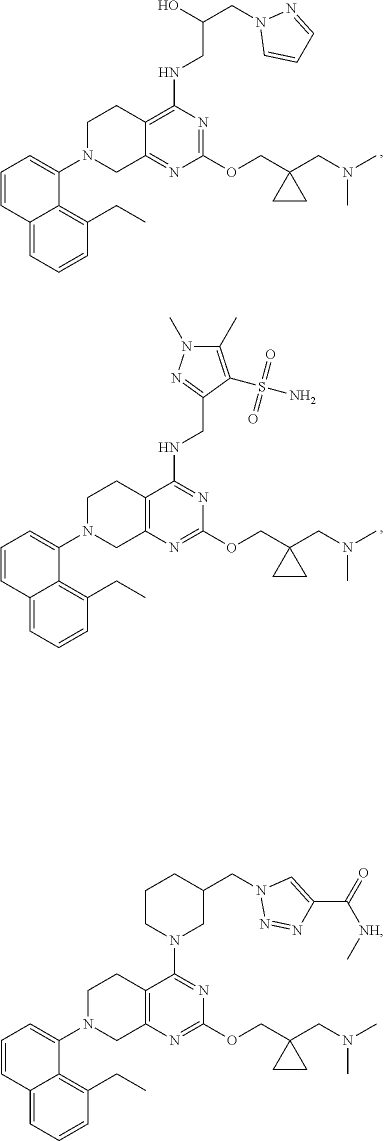 [SMILES: CCc1cccc2cccc(N3CCc4c(nc(OCC5(CN(C)C)CC5)nc4NCC(O)Cn4cccn4)C3)c12 ; CCc1cccc2cccc(N3CCc4c(nc(OCC5(CN(C)C)CC5)nc4NCc4nn(C)c(C)c4S(N)(=O)=O)C3)c12 ; CCc1cccc2cccc(N3CCc4c(nc(OCC5(CN(C)C)CC5)nc4N4CCCC(Cn5cc(C(=O)NC)nn5)C4)C3)c12]

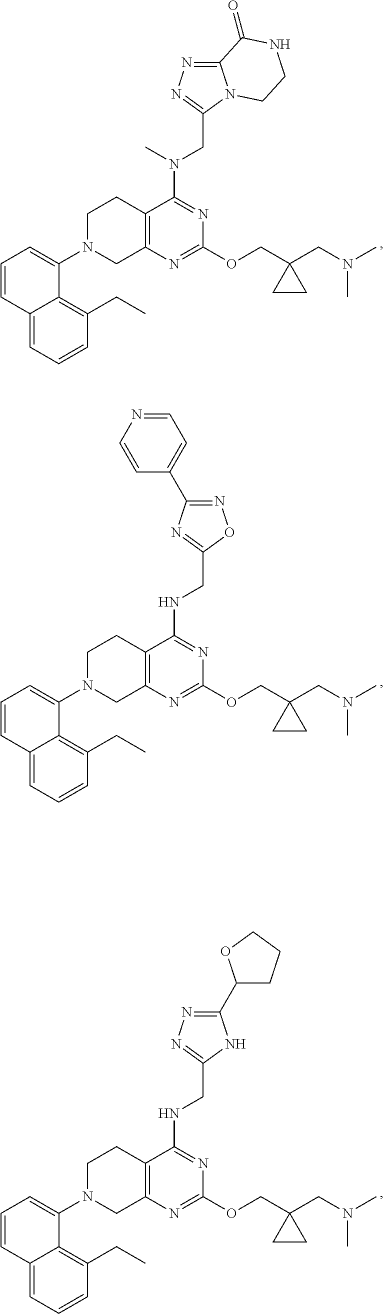 [SMILES: CCc1cccc2cccc(N3CCc4c(nc(OCC5(CN(C)C)CC5)nc4N(C)Cc4nnc5n4CCNC5=O)C3)c12 ; CCc1cccc2cccc(N3CCc4c(nc(OCC5(CN(C)C)CC5)nc4NCc4nc(-c5ccncc5)no4)C3)c12 ; CCc1cccc2cccc(N3CCc4c(nc(OCC5(CN(C)C)CC5)nc4NCc4nnc(C5CCCO5)[nH]4)C3)c12]

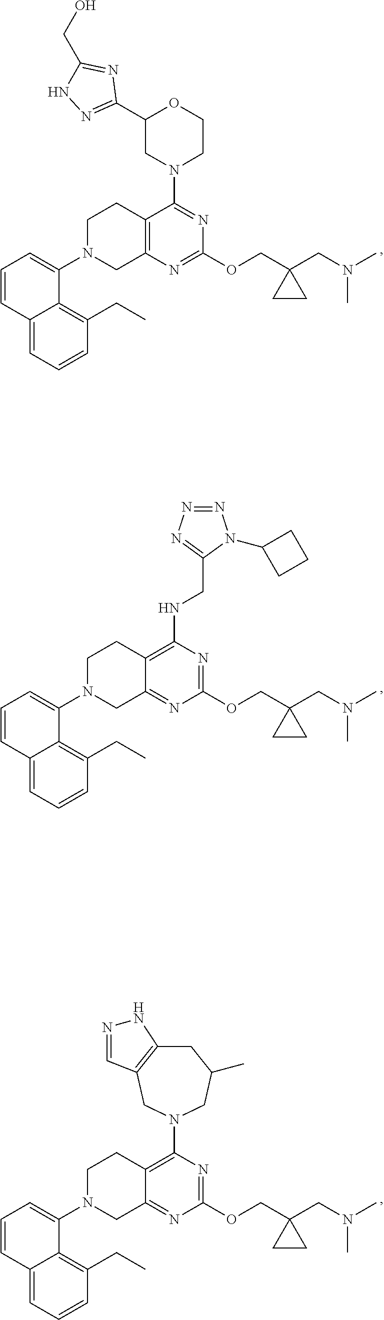 [SMILES: CCc1cccc2cccc(N3CCc4c(nc(OCC5(CN(C)C)CC5)nc4N4CCOC(c5n[nH]c(CO)n5)C4)C3)c12 ; CCc1cccc2cccc(N3CCc4c(nc(OCC5(CN(C)C)CC5)nc4NCc4nnnn4C4CCC4)C3)c12 ; CCc1cccc2cccc(N3CCc4c(nc(OCC5(CN(C)C)CC5)nc4N4Cc5cn[nH]c5CC(C)C4)C3)c12]

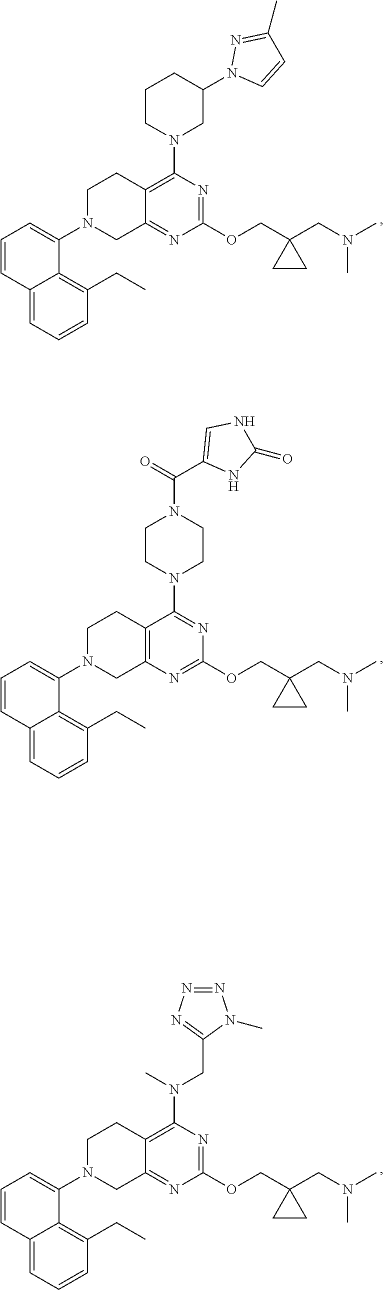 [SMILES: CCc1cccc2cccc(N3CCc4c(nc(OCC5(CN(C)C)CC5)nc4N4CCCC(n5ccc(C)n5)C4)C3)c12 ; CCc1cccc2cccc(N3CCc4c(nc(OCC5(CN(C)C)CC5)nc4N4CCN(C(=O)c5c[nH]c(=O)[nH]5)CC4)C3)c12 ; CCc1cccc2cccc(N3CCc4c(nc(OCC5(CN(C)C)CC5)nc4N(C)Cc4nnnn4C)C3)c12]

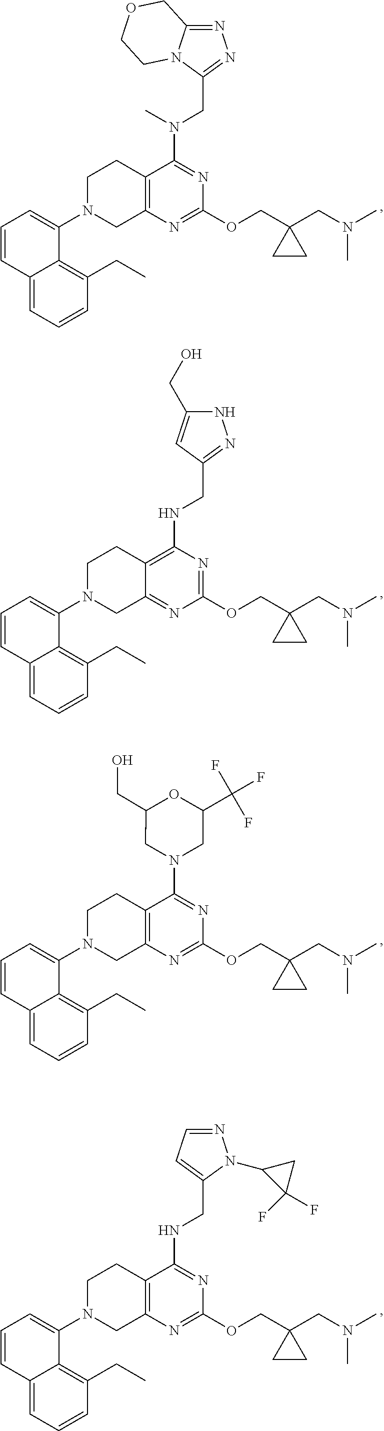 [SMILES: CCc1cccc2cccc(N3CCc4c(nc(OCC5(CN(C)C)CC5)nc4N(C)Cc4nnc5n4CCOC5)C3)c12 ; CCc1cccc2cccc(N3CCc4c(nc(OCC5(CN(C)C)CC5)nc4NCc4cc(CO)[nH]n4)C3)c12 ; CCc1cccc2cccc(N3CCc4c(nc(OCC5(CN(C)C)CC5)nc4N4CC(CO)OC(C(F)(F)F)C4)C3)c12 ; CCc1cccc2cccc(N3CCc4c(nc(OCC5(CN(C)C)CC5)nc4NCc4ccnn4C4CC4(F)F)C3)c12]

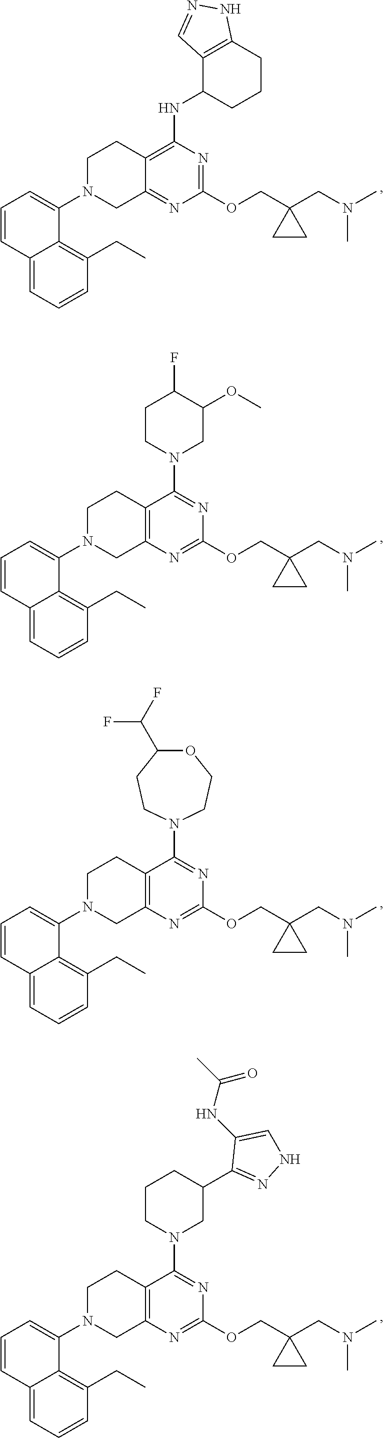 [SMILES: CCc1cccc2cccc(N3CCc4c(nc(OCC5(CN(C)C)CC5)nc4NC4CCCc5[nH]ncc54)C3)c12 ; CCc1cccc2cccc(N3CCc4c(nc(OCC5(CN(C)C)CC5)nc4N4CCC(F)C(OC)C4)C3)c12 ; CCc1cccc2cccc(N3CCc4c(nc(OCC5(CN(C)C)CC5)nc4N4CCOC(C(F)F)CC4)C3)c12 ; CCc1cccc2cccc(N3CCc4c(nc(OCC5(CN(C)C)CC5)nc4N4CCCC(c5n[nH]cc5NC(C)=O)C4)C3)c12]

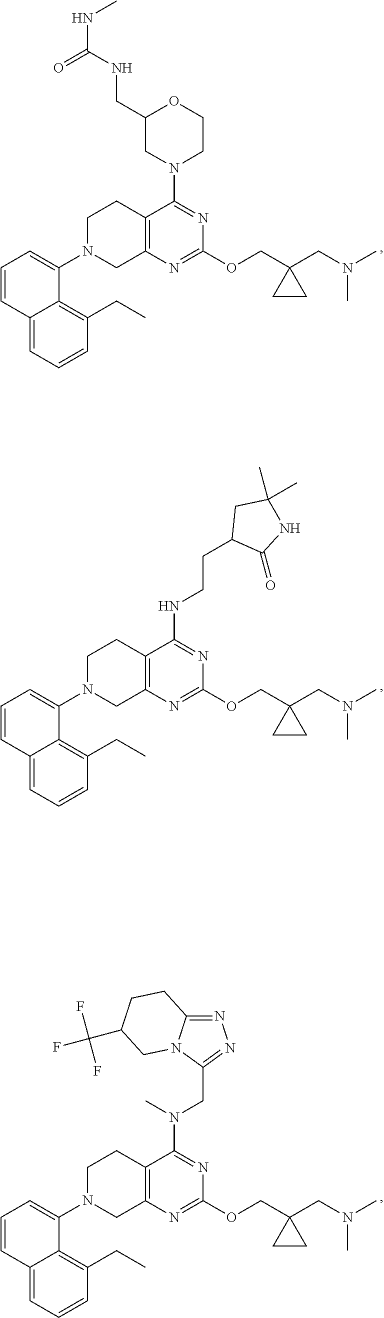 [SMILES: CCc1cccc2cccc(N3CCc4c(nc(OCC5(CN(C)C)CC5)nc4N4CCOC(CNC(=O)NC)C4)C3)c12 ; CCc1cccc2cccc(N3CCc4c(nc(OCC5(CN(C)C)CC5)nc4NCCC4CC(C)(C)NC4=O)C3)c12 ; CCc1cccc2cccc(N3CCc4c(nc(OCC5(CN(C)C)CC5)nc4N(C)Cc4nnc5n4CC(C(F)(F)F)CC5)C3)c12]

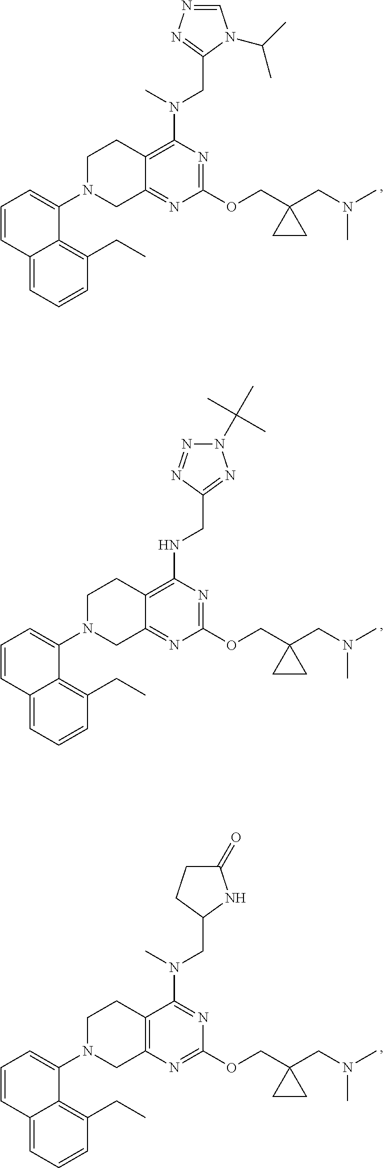 [SMILES: CCc1cccc2cccc(N3CCc4c(nc(OCC5(CN(C)C)CC5)nc4N(C)Cc4nncn4C(C)C)C3)c12 ; CCc1cccc2cccc(N3CCc4c(nc(OCC5(CN(C)C)CC5)nc4NCc4nnn(C(C)(C)C)n4)C3)c12 ; CCc1cccc2cccc(N3CCc4c(nc(OCC5(CN(C)C)CC5)nc4N(C)CC4CCC(=O)N4)C3)c12]

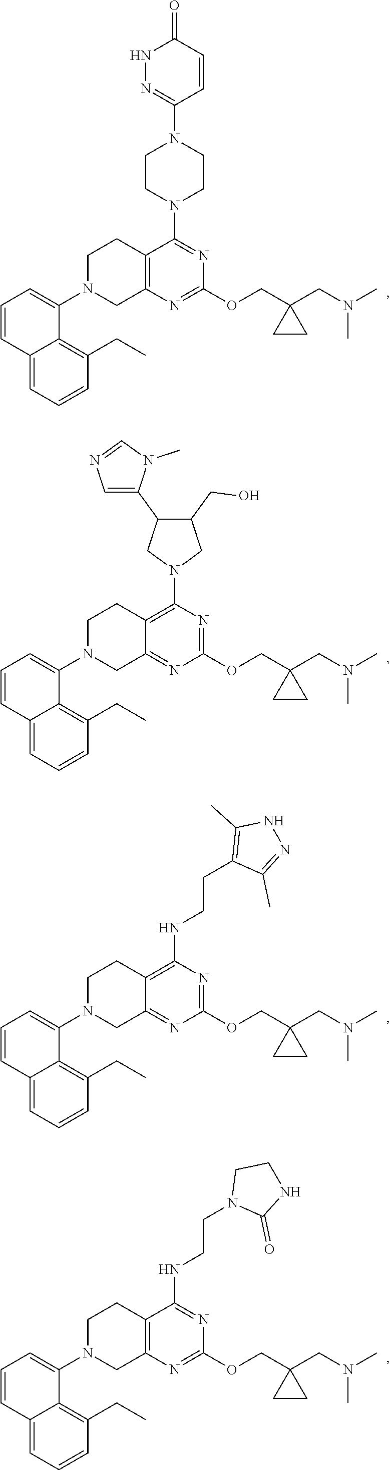 [SMILES: CCc1cccc2cccc(N3CCc4c(nc(OCC5(CN(C)C)CC5)nc4N4CCN(c5ccc(=O)[nH]n5)CC4)C3)c12 ; CCc1cccc2cccc(N3CCc4c(nc(OCC5(CN(C)C)CC5)nc4N4CC(CO)C(c5cncn5C)C4)C3)c12 ; CCc1cccc2cccc(N3CCc4c(nc(OCC5(CN(C)C)CC5)nc4NCCc4c(C)n[nH]c4C)C3)c12 ; CCc1cccc2cccc(N3CCc4c(nc(OCC5(CN(C)C)CC5)nc4NCCN4CCNC4=O)C3)c12]

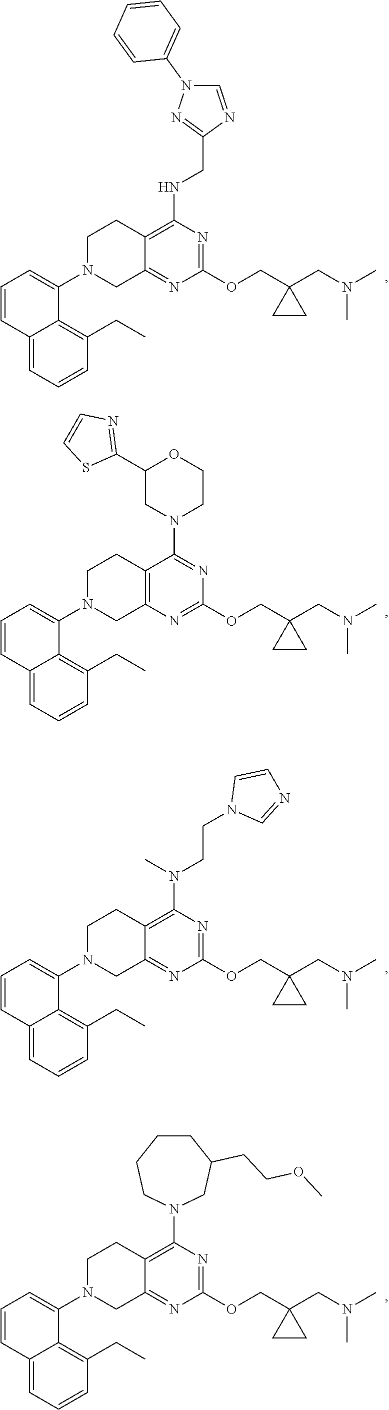

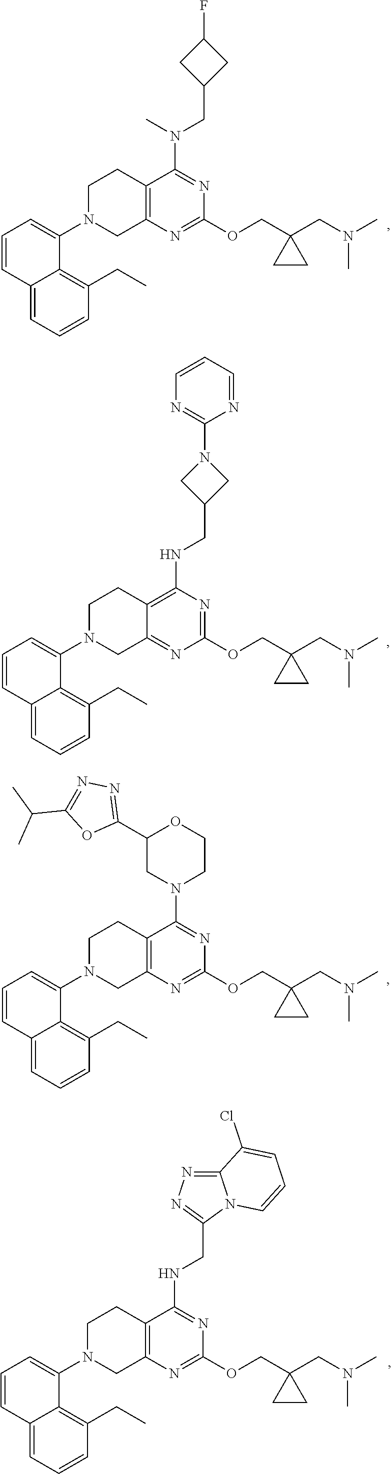

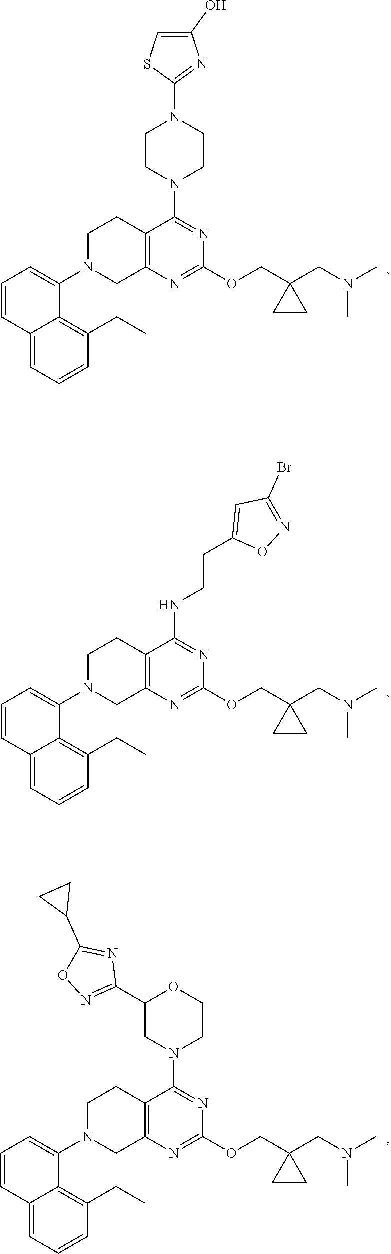 [SMILES: CCc1cccc2cccc(N3CCc4c(nc(OCC5(CN(C)C)CC5)nc4N4CCN(c5nc(O)cs5)CC4)C3)c12 ; CCc1cccc2cccc(N3CCc4c(nc(OCC5(CN(C)C)CC5)nc4NCCc4cc(Br)no4)C3)c12 ; CCc1cccc2cccc(N3CCc4c(nc(OCC5(CN(C)C)CC5)nc4N4CCOC(c5noc(C6CC6)n5)C4)C3)c12]

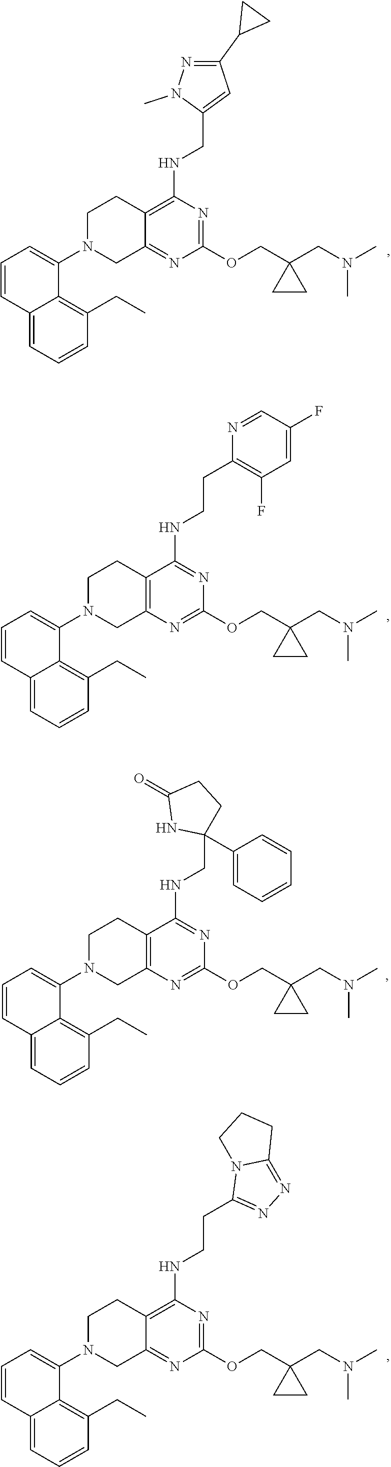 [SMILES: CCc1cccc2cccc(N3CCc4c(nc(OCC5(CN(C)C)CC5)nc4NCc4cc(C5CC5)nn4C)C3)c12 ; CCc1cccc2cccc(N3CCc4c(nc(OCC5(CN(C)C)CC5)nc4NCCc4ncc(F)cc4F)C3)c12 ; CCc1cccc2cccc(N3CCc4c(nc(OCC5(CN(C)C)CC5)nc4NCC4(c5ccccc5)CCC(=O)N4)C3)c12 ; CCc1cccc2cccc(N3CCc4c(nc(OCC5(CN(C)C)CC5)nc4NCCc4nnc5n4CCC5)C3)c12]

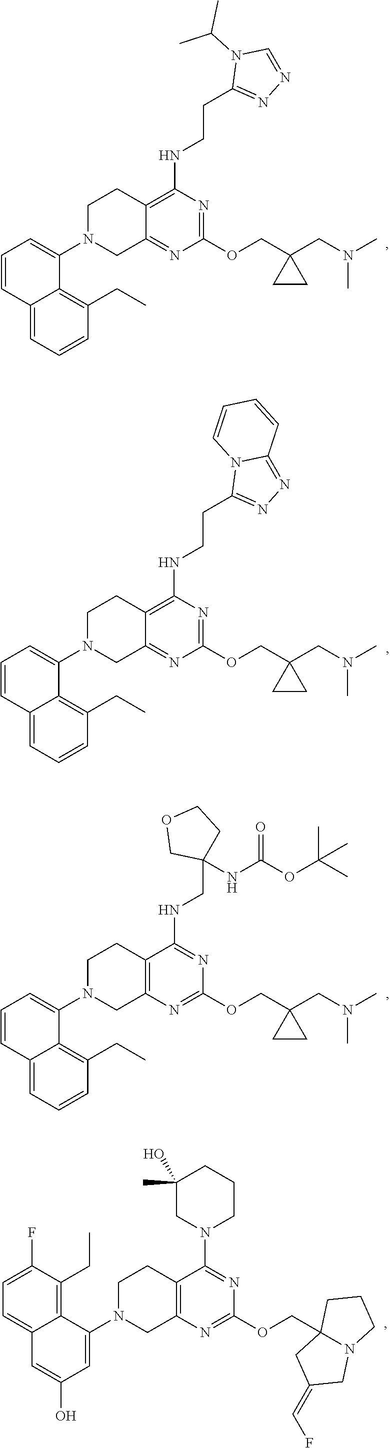 [SMILES: CCc1cccc2cccc(N3CCc4c(nc(OCC5(CN(C)C)CC5)nc4NCCc4nncn4C(C)C)C3)c12 ; CCc1cccc2cccc(N3CCc4c(nc(OCC5(CN(C)C)CC5)nc4NCCc4nnc5ccccn45)C3)c12 ; CCc1cccc2cccc(N3CCc4c(nc(OCC5(CN(C)C)CC5)nc4NCC4(NC(=O)OC(C)(C)C)CCOC4)C3)c12 ; CCc1c(F)ccc2cc(O)cc(N3CCc4c(nc(OCC56CCCN5C/C(=C\F)C6)nc4N4CCC[C@@](C)(O)C4)C3)c12]

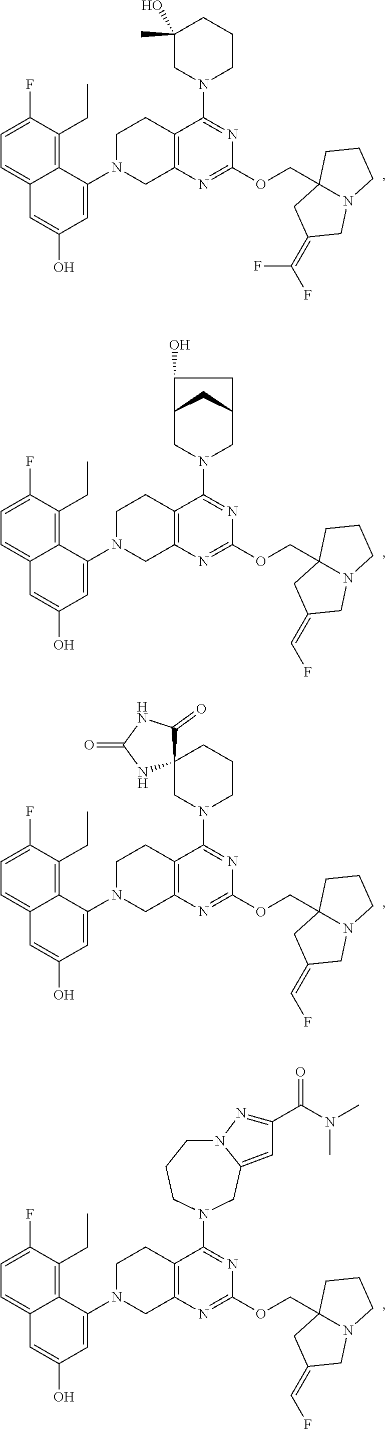 [SMILES: CCc1c(F)ccc2cc(O)cc(N3CCc4c(nc(OCC56CCCN5CC(=C(F)F)C6)nc4N4CCC[C@@](C)(O)C4)C3)c12 ; CCc1c(F)ccc2cc(O)cc(N3CCc4c(nc(OCC56CCCN5C/C(=C\F)C6)nc4N4C[C@@H]5C[C@H](C4)[C@H](O)C5)C3)c12 ; CCc1c(F)ccc2cc(O)cc(N3CCc4c(nc(OCC56CCCN5C/C(=C\F)C6)nc4N4CCC[C@]5(C4)NC(=O)NC5=O)C3)c12 ; CCc1c(F)ccc2cc(O)cc(N3CCc4c(nc(OCC56CCCN5C/C(=C\F)C6)nc4N4CCCn5nc(C(=O)N(C)C)cc5C4)C3)c12]

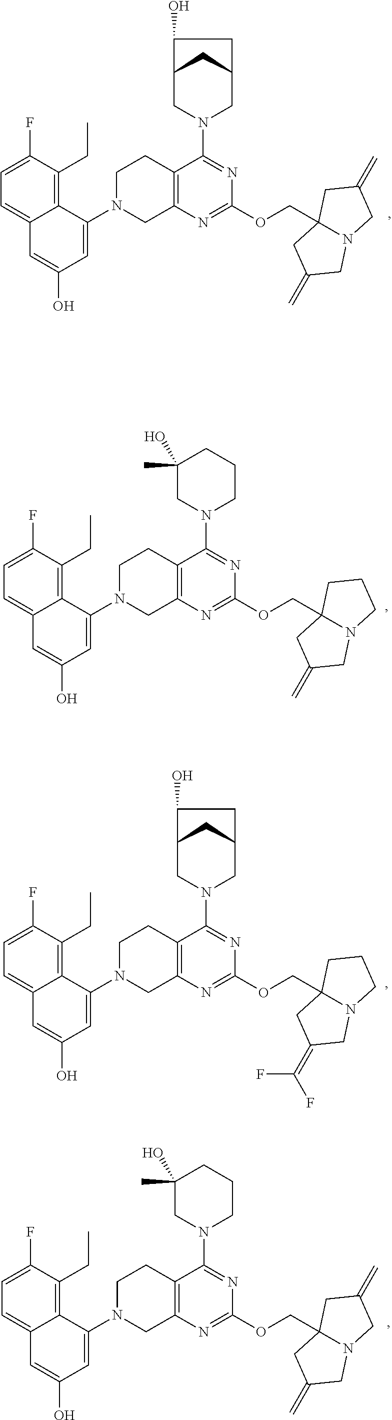 [SMILES: C=C1CN2CC(=C)CC2(COc2nc3c(c(N4C[C@@H]5C[C@H](C4)[C@H](O)C5)n2)CCN(c2cc(O)cc4ccc(F)c(CC)c24)C3)C1 ; C=C1CN2CCCC2(COc2nc3c(c(N4CCC[C@@](C)(O)C4)n2)CCN(c2cc(O)cc4ccc(F)c(CC)c24)C3)C1 ; CCc1c(F)ccc2cc(O)cc(N3CCc4c(nc(OCC56CCCN5CC(=C(F)F)C6)nc4N4C[C@@H]5C[C@H](C4)[C@H](O)C5)C3)c12 ; C=C1CN2CC(=C)CC2(COc2nc3c(c(N4CCC[C@@](C)(O)C4)n2)CCN(c2cc(O)cc4ccc(F)c(CC)c24)C3)C1]

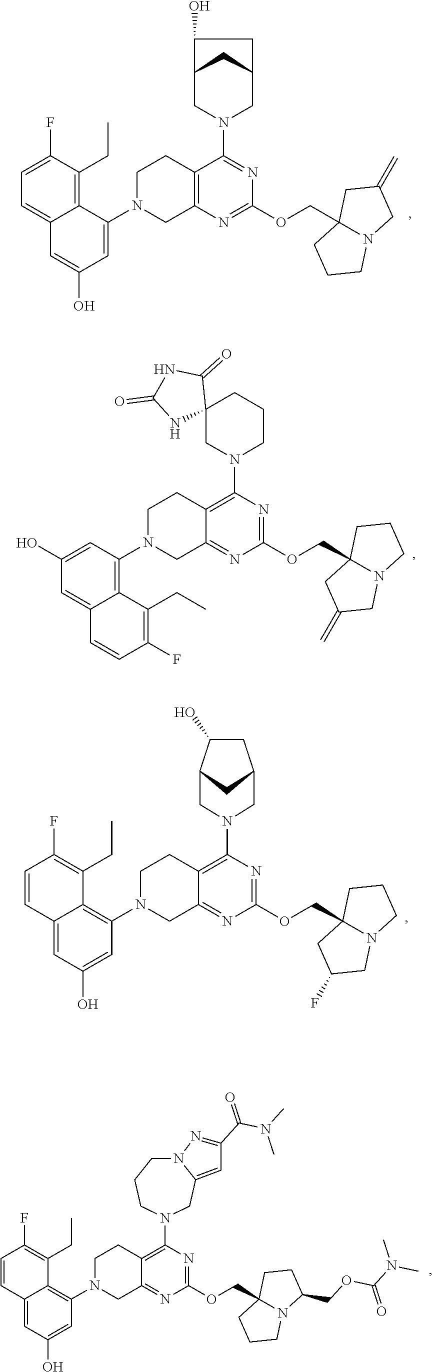 [SMILES: C=C1CN2CCCC2(COc2nc3c(c(N4C[C@@H]5C[C@H](C4)[C@H](O)C5)n2)CCN(c2cc(O)cc4ccc(F)c(CC)c24)C3)C1 ; C=C1CN2CCC[C@@]2(COc2nc3c(c(N4CCC[C@]5(C4)NC(=O)NC5=O)n2)CCN(c2cc(O)cc4ccc(F)c(CC)c24)C3)C1 ; CCc1c(F)ccc2cc(O)cc(N3CCc4c(nc(OC[C@@]56CCCN5C[C@H](F)C6)nc4N4C[C@@H]5C[C@H](C4)[C@H](O)C5)C3)c12 ; CCc1c(F)ccc2cc(O)cc(N3CCc4c(nc(OC[C@]56CCCN5[C@H](COC(=O)N(C)C)CC6)nc4N4CCCn5nc(C(=O)N(C)C)cc5C4)C3)c12]

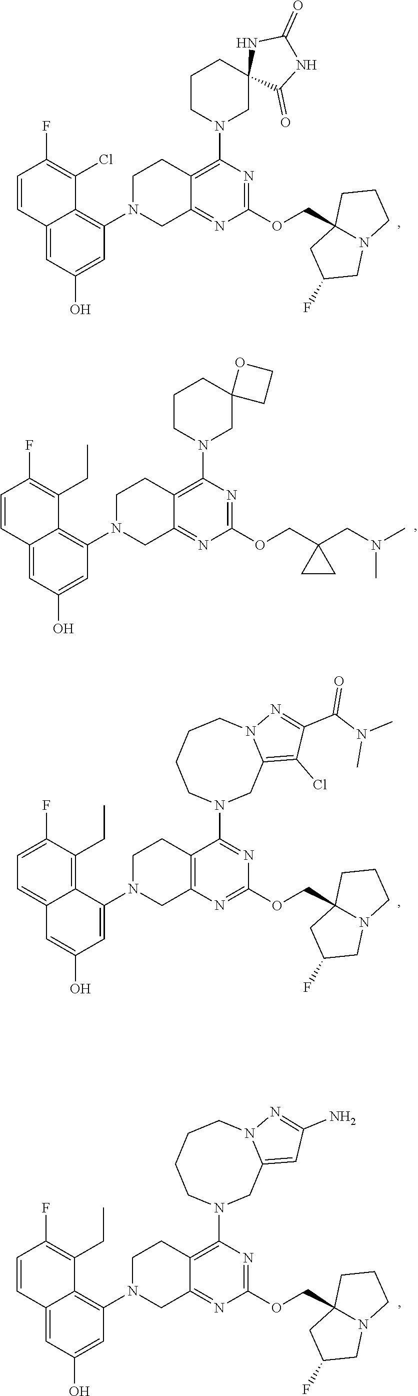 [SMILES: O=C1NC(=O)[C@]2(CCCN(c3nc(OC[C@@]45CCCN4C[C@H](F)C5)nc4c3CCN(c3cc(O)cc5ccc(F)c(Cl)c35)C4)C2)N1 ; CCc1c(F)ccc2cc(O)cc(N3CCc4c(nc(OCC5(CN(C)C)CC5)nc4N4CCCC5(CCO5)C4)C3)c12 ; CCc1c(F)ccc2cc(O)cc(N3CCc4c(nc(OC[C@@]56CCCN5C[C@H](F)C6)nc4N4CCCCn5nc(C(=O)N(C)C)c(Cl)c5C4)C3)c12 ; CCc1c(F)ccc2cc(O)cc(N3CCc4c(nc(OC[C@@]56CCCN5C[C@H](F)C6)nc4N4CCCCn5nc(N)cc5C4)C3)c12]

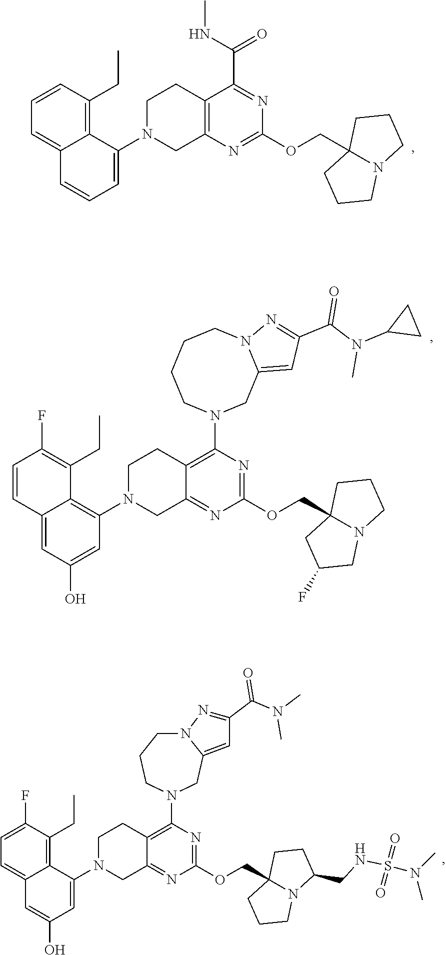 [SMILES: CCc1cccc2cccc(N3CCc4c(nc(OCC56CCCN5CCC6)nc4C(=O)NC)C3)c12 ; CCc1c(F)ccc2cc(O)cc(N3CCc4c(nc(OC[C@@]56CCCN5C[C@H](F)C6)nc4N4CCCCn5nc(C(=O)N(C)C6CC6)cc5C4)C3)c12 ; CCc1c(F)ccc2cc(O)cc(N3CCc4c(nc(OC[C@]56CCCN5[C@H](CNS(=O)(=O)N(C)C)CC6)nc4N4CCCn5nc(C(=O)N(C)C)cc5C4)C3)c12]

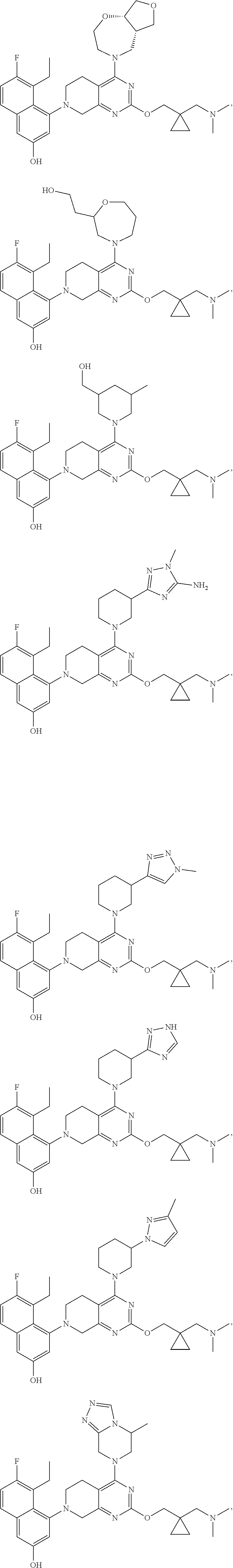

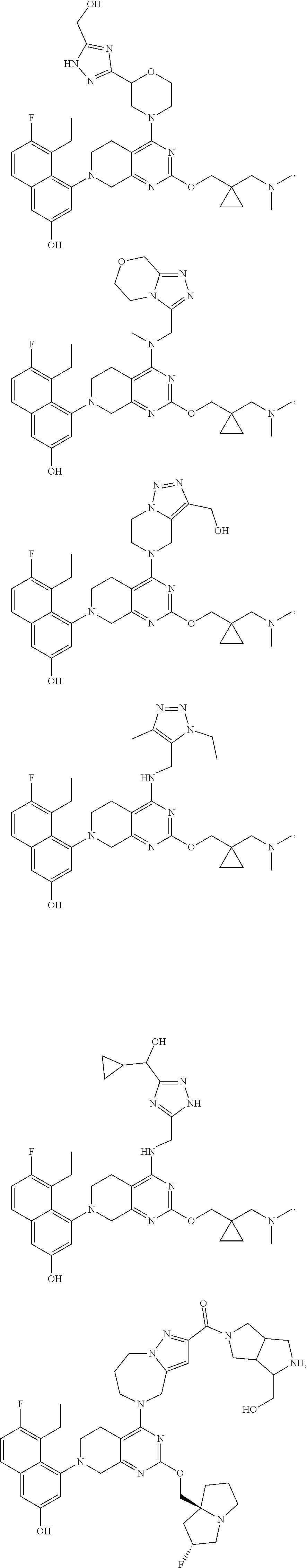 [SMILES: CCc1c(F)ccc2cc(O)cc(N3CCc4c(nc(OCC5(CN(C)C)CC5)nc4N4CCOC(c5n[nH]c(CO)n5)C4)C3)c12 ; CCc1c(F)ccc2cc(O)cc(N3CCc4c(nc(OCC5(CN(C)C)CC5)nc4N(C)Cc4nnc5n4CCOC5)C3)c12 ; CCc1c(F)ccc2cc(O)cc(N3CCc4c(nc(OCC5(CN(C)C)CC5)nc4N4CCn5nnc(CO)c5C4)C3)c12 ; CCc1c(F)ccc2cc(O)cc(N3CCc4c(nc(OCC5(CN(C)C)CC5)nc4NCc4c(C)nnn4CC)C3)c12 ; CCc1c(F)ccc2cc(O)cc(N3CCc4c(nc(OCC5(CN(C)C)CC5)nc4NCc4nc(C(O)C5CC5)n[nH]4)C3)c12 ; CCc1c(F)ccc2cc(O)cc(N3CCc4c(nc(OC[C@@]56CCCN5C[C@H](F)C6)nc4N4CCCn5nc(C(=O)N6CC7CNC(CO)C7C6)cc5C4)C3)c12]

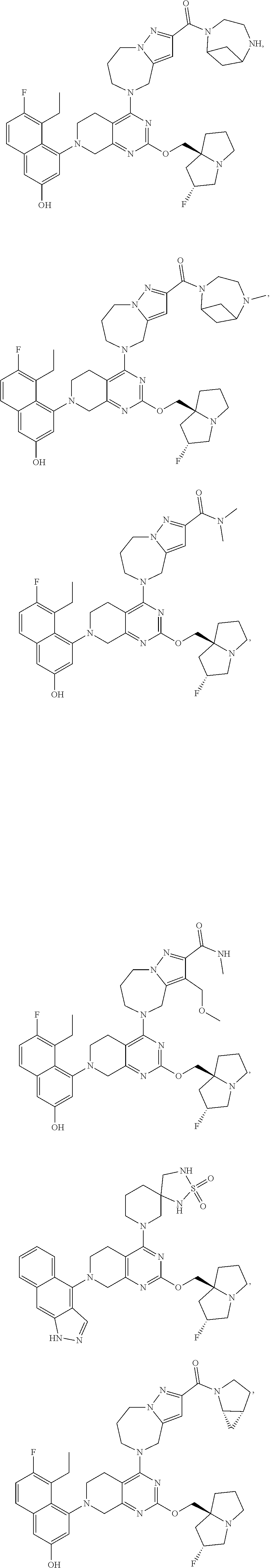

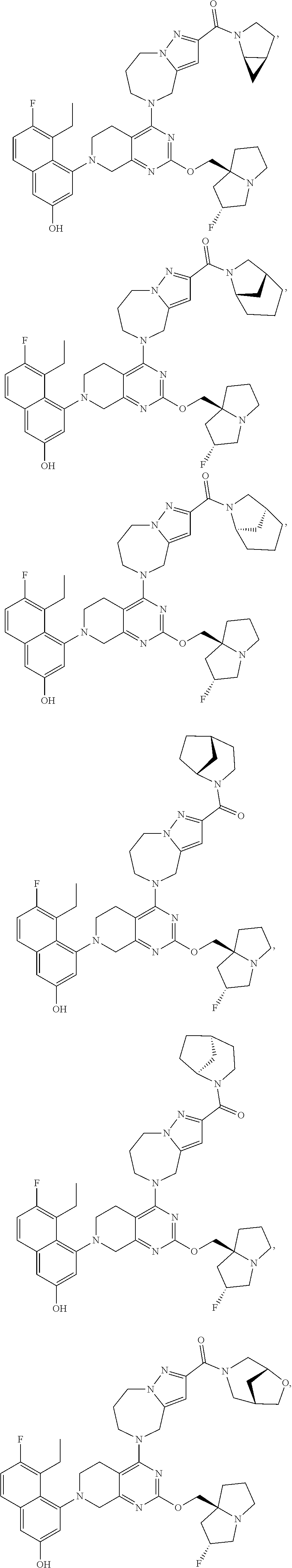

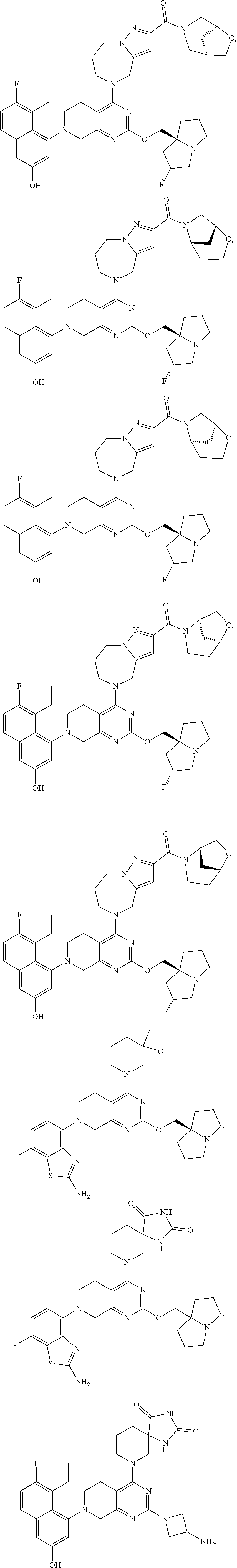

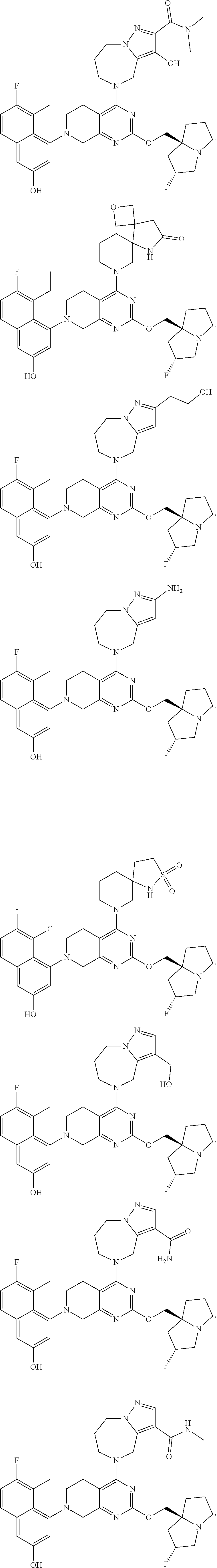

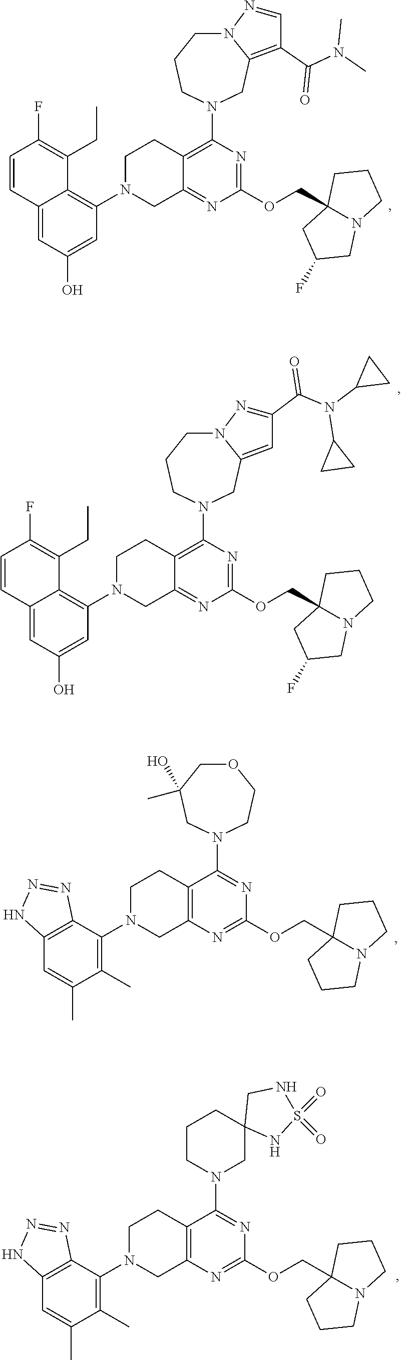 [SMILES: CCc1c(F)ccc2cc(O)cc(N3CCc4c(nc(OC[C@@]56CCCN5C[C@H](F)C6)nc4N4CCCn5ncc(C(=O)N(C)C)c5C4)C3)c12 ; CCc1c(F)ccc2cc(O)cc(N3CCc4c(nc(OC[C@@]56CCCN5C[C@H](F)C6)nc4N4CCCn5nc(C(=O)N(C6CC6)C6CC6)cc5C4)C3)c12 ; Cc1cc2[nH]nnc2c(N2CCc3c(nc(OCC45CCCN4CCC5)nc3N3CCOC[C@@](C)(O)C3)C2)c1C ; Cc1cc2[nH]nnc2c(N2CCc3c(nc(OCC45CCCN4CCC5)nc3N3CCCC4(CNS(=O)(=O)N4)C3)C2)c1C]

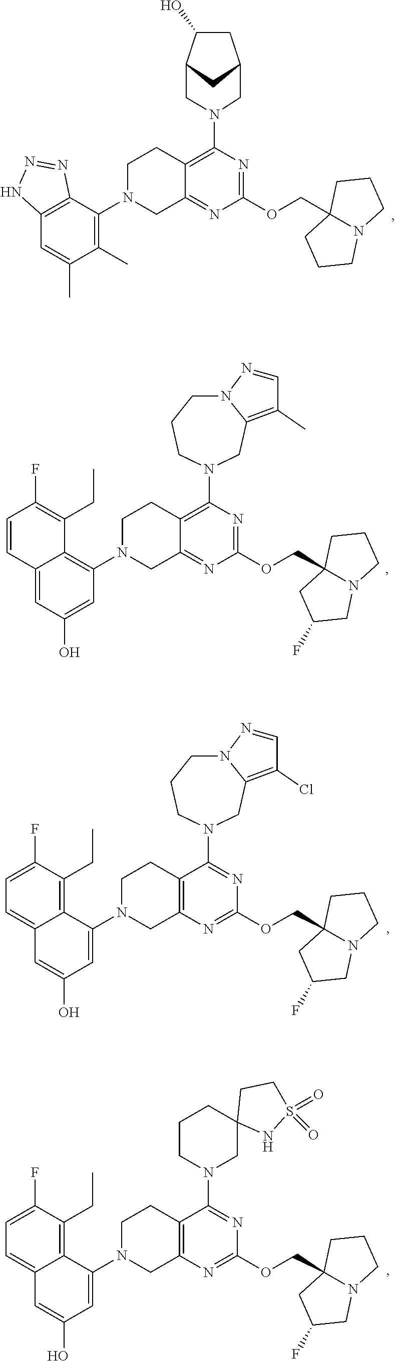 [SMILES: Cc1cc2[nH]nnc2c(N2CCc3c(nc(OCC45CCCN4CCC5)nc3N3C[C@@H]4C[C@H](C3)[C@H](O)C4)C2)c1C ; CCc1c(F)ccc2cc(O)cc(N3CCc4c(nc(OC[C@@]56CCCN5C[C@H](F)C6)nc4N4CCCn5ncc(C)c5C4)C3)c12 ; CCc1c(F)ccc2cc(O)cc(N3CCc4c(nc(OC[C@@]56CCCN5C[C@H](F)C6)nc4N4CCCn5ncc(Cl)c5C4)C3)c12 ; CCc1c(F)ccc2cc(O)cc(N3CCc4c(nc(OC[C@@]56CCCN5C[C@H](F)C6)nc4N4CCCC5(CCS(=O)(=O)N5)C4)C3)c12]

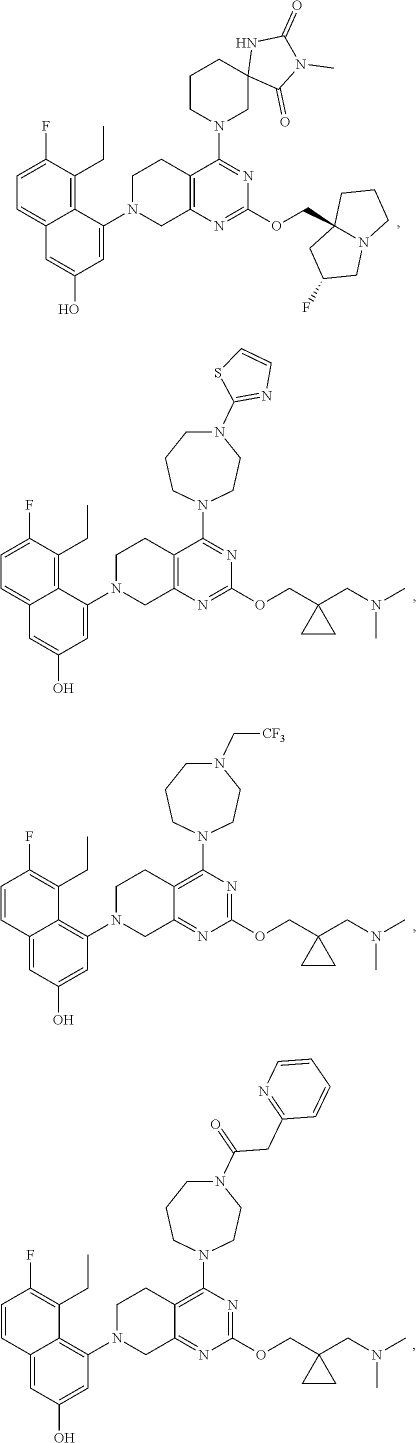 [SMILES: CCc1c(F)ccc2cc(O)cc(N3CCc4c(nc(OC[C@@]56CCCN5C[C@H](F)C6)nc4N4CCCC5(C4)NC(=O)N(C)C5=O)C3)c12 ; CCc1c(F)ccc2cc(O)cc(N3CCc4c(nc(OCC5(CN(C)C)CC5)nc4N4CCCN(c5nccs5)CC4)C3)c12 ; CCc1c(F)ccc2cc(O)cc(N3CCc4c(nc(OCC5(CN(C)C)CC5)nc4N4CCCN(CC(F)(F)F)CC4)C3)c12 ; CCc1c(F)ccc2cc(O)cc(N3CCc4c(nc(OCC5(CN(C)C)CC5)nc4N4CCCN(C(=O)Cc5ccccn5)CC4)C3)c12]

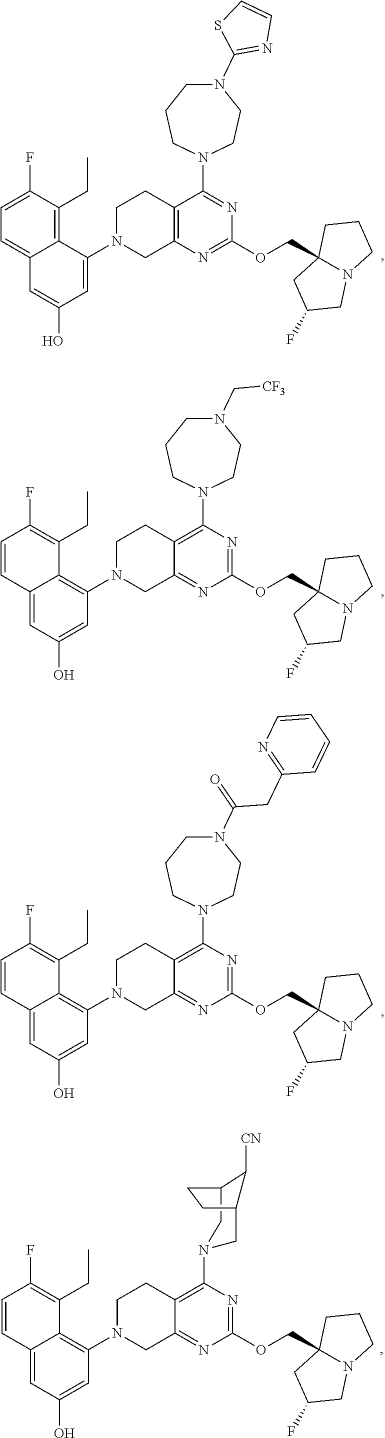

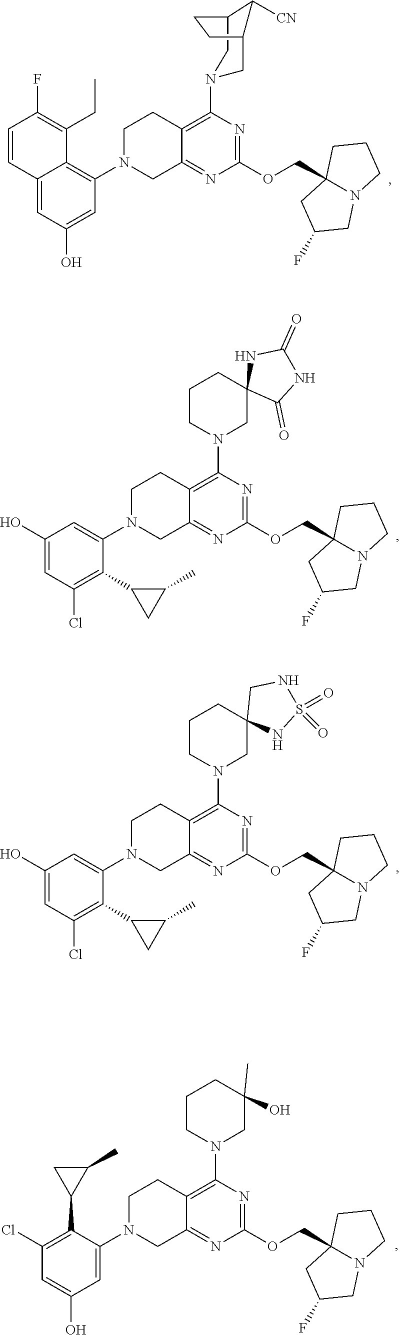

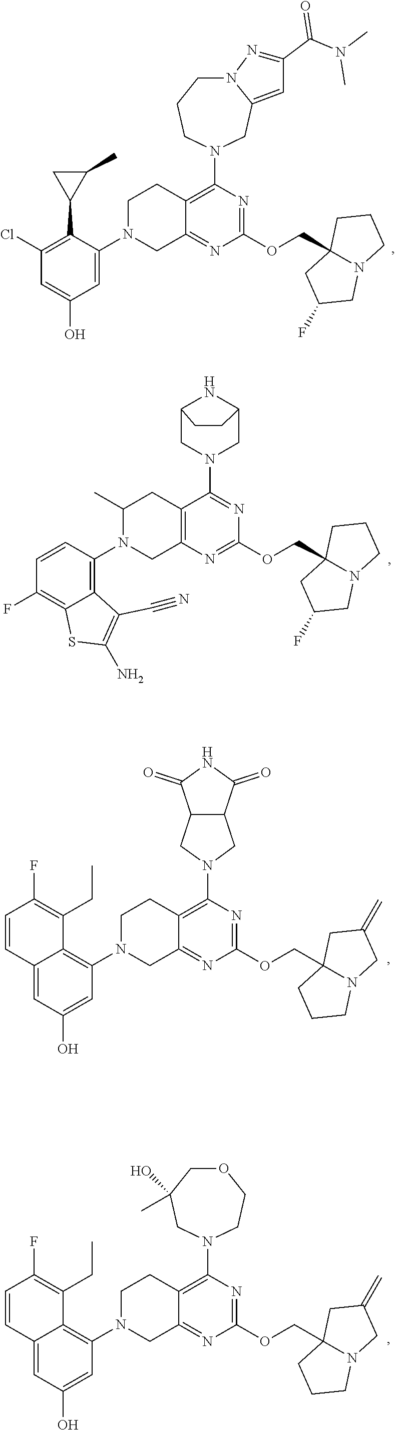 [SMILES: C[C@@H]1C[C@@H]1c1c(Cl)cc(O)cc1N1CCc2c(nc(OC[C@@]34CCCN3C[C@H](F)C4)nc2N2CCCn3nc(C(=O)N(C)C)cc3C2)C1 ; CC1Cc2c(nc(OC[C@@]34CCCN3C[C@H](F)C4)nc2N2CC3CCC(C2)N3)CN1c1ccc(F)c2sc(N)c(C#N)c12 ; C=C1CN2CCCC2(COc2nc3c(c(N4CC5C(=O)NC(=O)C5C4)n2)CCN(c2cc(O)cc4ccc(F)c(CC)c24)C3)C1 ; C=C1CN2CCCC2(COc2nc3c(c(N4CCOC[C@@](C)(O)C4)n2)CCN(c2cc(O)cc4ccc(F)c(CC)c24)C3)C1]

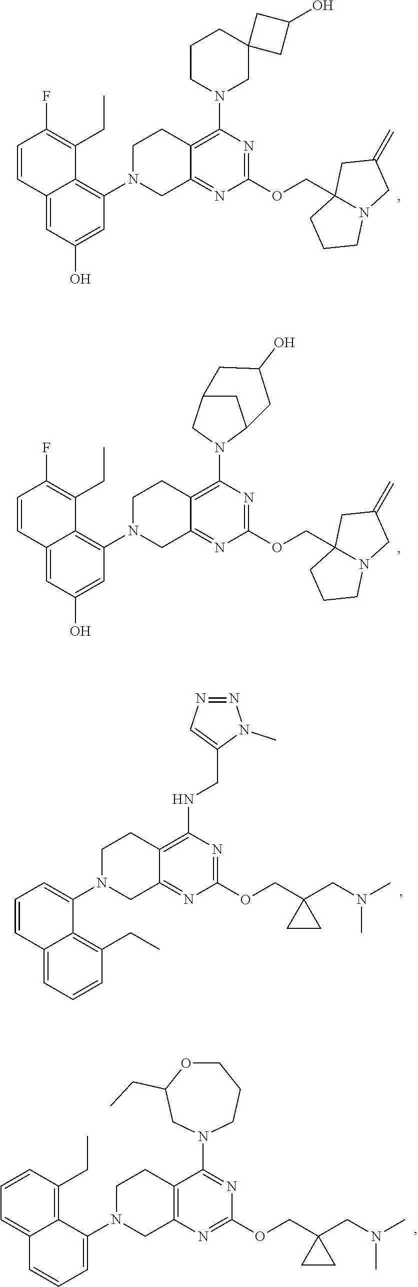 [SMILES: C=C1CN2CCCC2(COc2nc3c(c(N4CCCC5(CC(O)C5)C4)n2)CCN(c2cc(O)cc4ccc(F)c(CC)c24)C3)C1 ; C=C1CN2CCCC2(COc2nc3c(c(N4CC5CC(O)CC4C5)n2)CCN(c2cc(O)cc4ccc(F)c(CC)c24)C3)C1 ; CCc1cccc2cccc(N3CCc4c(nc(OCC5(CN(C)C)CC5)nc4NCc4cnnn4C)C3)c12 ; CCc1cccc2cccc(N3CCc4c(nc(OCC5(CN(C)C)CC5)nc4N4CCCOC(CC)C4)C3)c12]

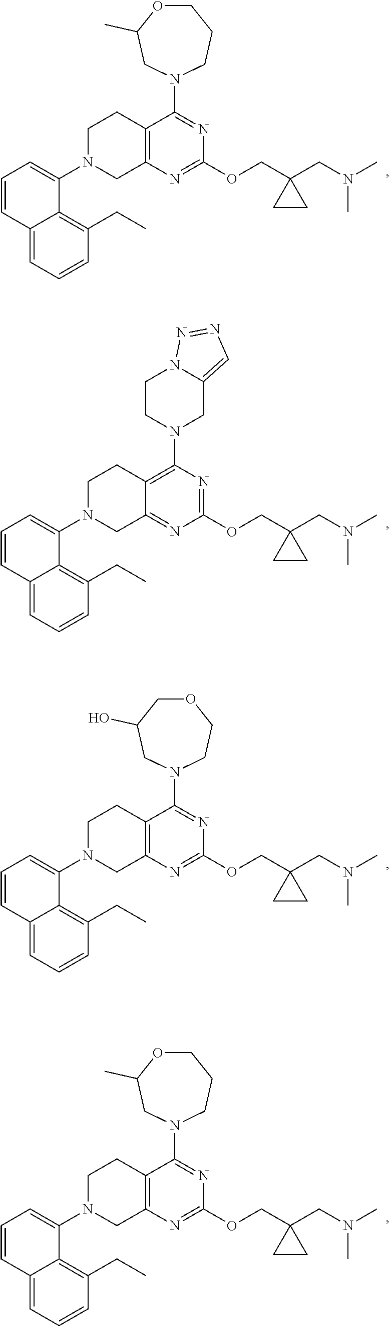 [SMILES: CCc1cccc2cccc(N3CCc4c(nc(OCC5(CN(C)C)CC5)nc4N4CCCOC(C)C4)C3)c12 ; CCc1cccc2cccc(N3CCc4c(nc(OCC5(CN(C)C)CC5)nc4N4CCn5nncc5C4)C3)c12 ; CCc1cccc2cccc(N3CCc4c(nc(OCC5(CN(C)C)CC5)nc4N4CCOCC(O)C4)C3)c12 ; CCc1cccc2cccc(N3CCc4c(nc(OCC5(CN(C)C)CC5)nc4N4CCCOC(C)C4)C3)c12]

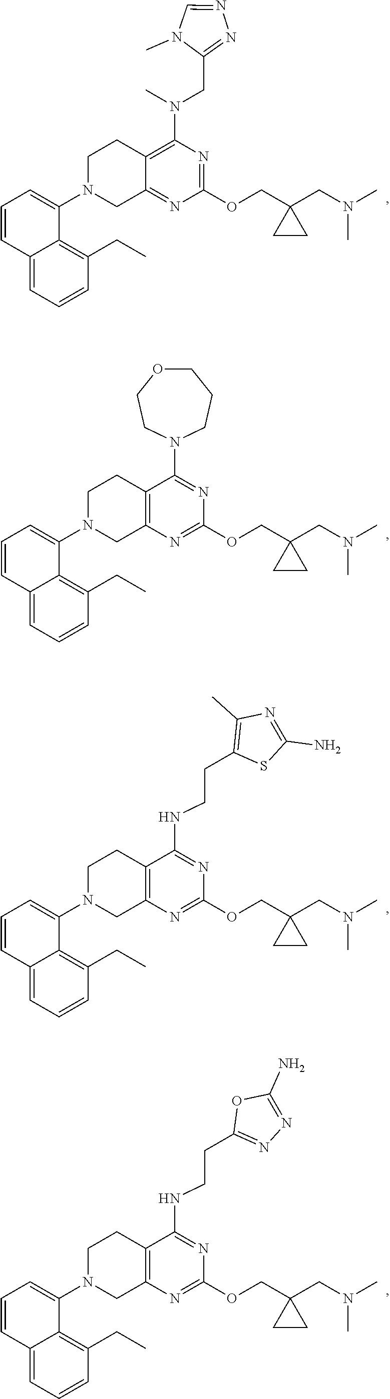 [SMILES: CCc1cccc2cccc(N3CCc4c(nc(OCC5(CN(C)C)CC5)nc4N(C)Cc4nncn4C)C3)c12 ; CCc1cccc2cccc(N3CCc4c(nc(OCC5(CN(C)C)CC5)nc4N4CCCOCC4)C3)c12 ; CCc1cccc2cccc(N3CCc4c(nc(OCC5(CN(C)C)CC5)nc4NCCc4sc(N)nc4C)C3)c12 ; CCc1cccc2cccc(N3CCc4c(nc(OCC5(CN(C)C)CC5)nc4NCCc4nnc(N)o4)C3)c12]

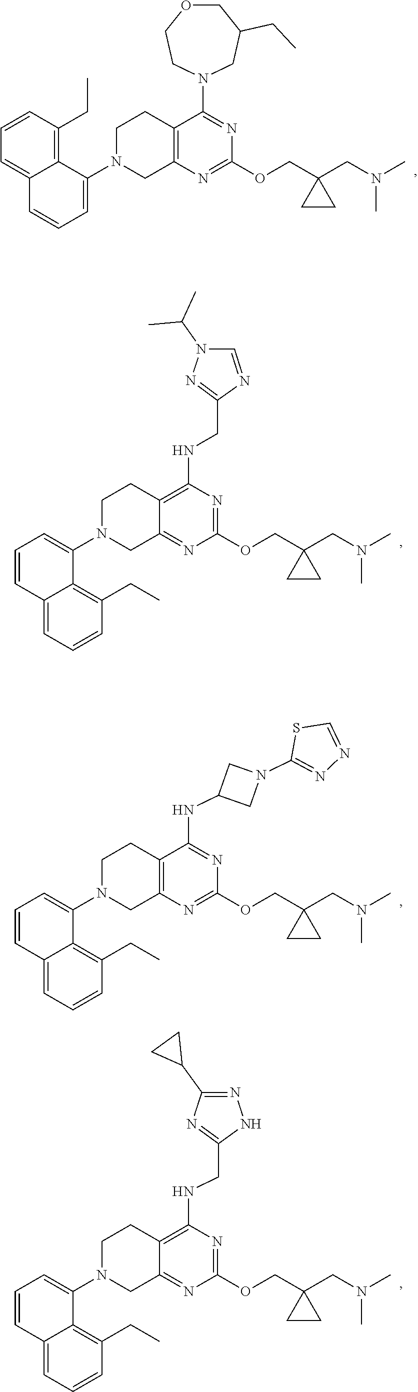 [SMILES: CCc1cccc2cccc(N3CCc4c(nc(OCC5(CN(C)C)CC5)nc4N4CCOCC(CC)C4)C3)c12 ; CCc1cccc2cccc(N3CCc4c(nc(OCC5(CN(C)C)CC5)nc4NCc4ncn(C(C)C)n4)C3)c12 ; CCc1cccc2cccc(N3CCc4c(nc(OCC5(CN(C)C)CC5)nc4NC4CN(c5nncs5)C4)C3)c12 ; CCc1cccc2cccc(N3CCc4c(nc(OCC5(CN(C)C)CC5)nc4NCc4nc(C5CC5)n[nH]4)C3)c12]

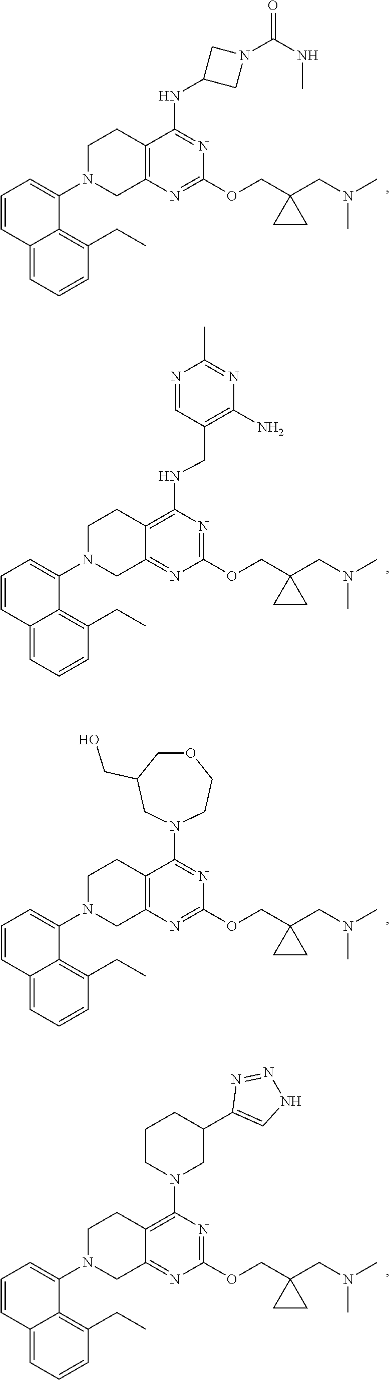 [SMILES: CCc1cccc2cccc(N3CCc4c(nc(OCC5(CN(C)C)CC5)nc4NC4CN(C(=O)NC)C4)C3)c12 ; CCc1cccc2cccc(N3CCc4c(nc(OCC5(CN(C)C)CC5)nc4NCc4cnc(C)nc4N)C3)c12 ; CCc1cccc2cccc(N3CCc4c(nc(OCC5(CN(C)C)CC5)nc4N4CCOCC(CO)C4)C3)c12 ; CCc1cccc2cccc(N3CCc4c(nc(OCC5(CN(C)C)CC5)nc4N4CCCC(c5c[nH]nn5)C4)C3)c12]

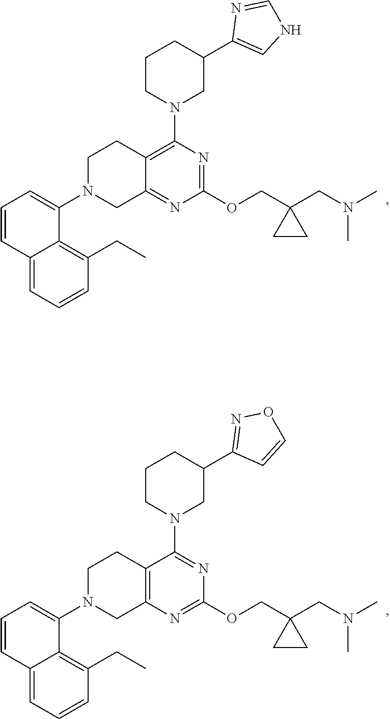 [SMILES: CCc1cccc2cccc(N3CCc4c(nc(OCC5(CN(C)C)CC5)nc4N4CCCC(c5c[nH]cn5)C4)C3)c12 ; CCc1cccc2cccc(N3CCc4c(nc(OCC5(CN(C)C)CC5)nc4N4CCCC(c5ccon5)C4)C3)c12]

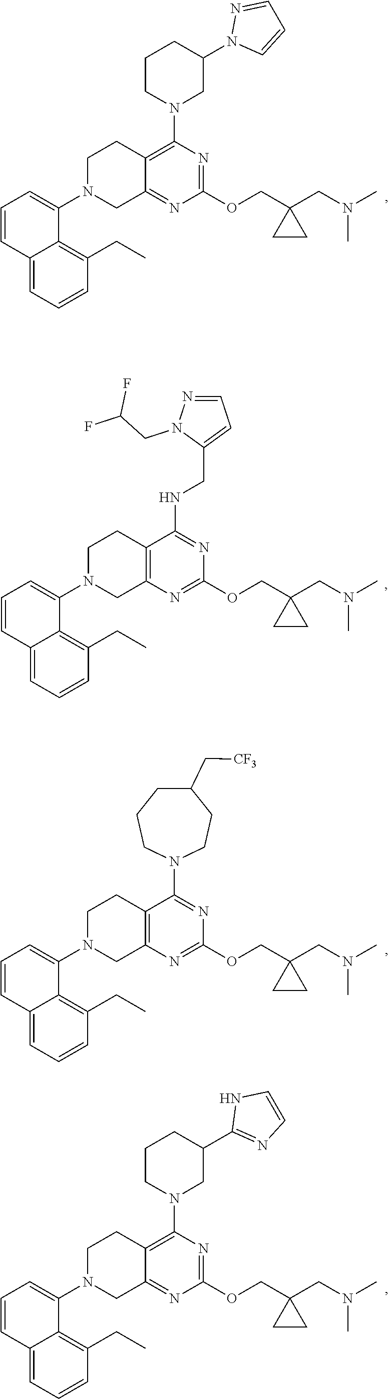 [SMILES: CCc1cccc2cccc(N3CCc4c(nc(OCC5(CN(C)C)CC5)nc4N4CCCC(n5cccn5)C4)C3)c12 ; CCc1cccc2cccc(N3CCc4c(nc(OCC5(CN(C)C)CC5)nc4NCc4ccnn4CC(F)F)C3)c12 ; CCc1cccc2cccc(N3CCc4c(nc(OCC5(CN(C)C)CC5)nc4N4CCCC(CC(F)(F)F)CC4)C3)c12 ; CCc1cccc2cccc(N3CCc4c(nc(OCC5(CN(C)C)CC5)nc4N4CCCC(c5ncc[nH]5)C4)C3)c12]

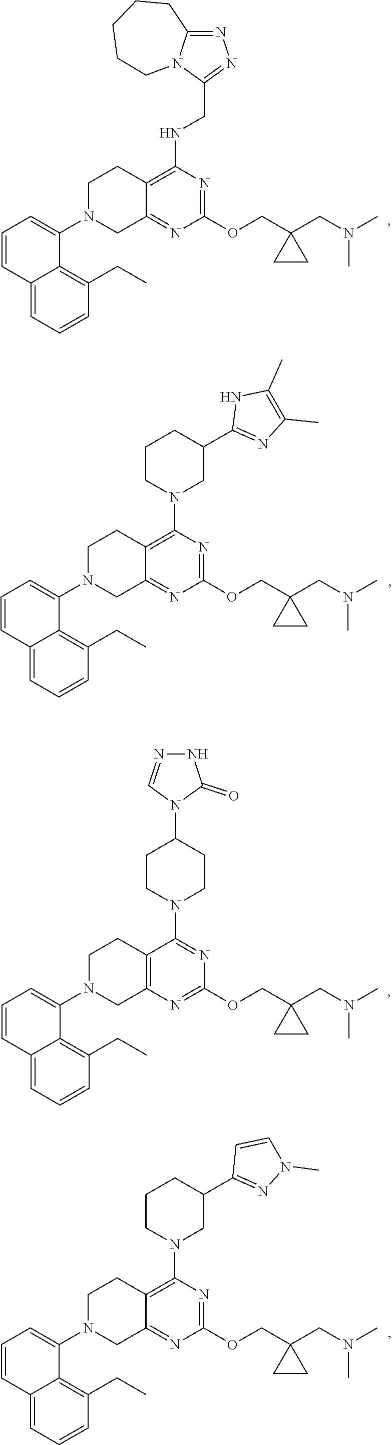 [SMILES: CCc1cccc2cccc(N3CCc4c(nc(OCC5(CN(C)C)CC5)nc4NCc4nnc5n4CCCCC5)C3)c12 ; CCc1cccc2cccc(N3CCc4c(nc(OCC5(CN(C)C)CC5)nc4N4CCCC(c5nc(C)c(C)[nH]5)C4)C3)c12 ; CCc1cccc2cccc(N3CCc4c(nc(OCC5(CN(C)C)CC5)nc4N4CCC(n5cn[nH]c5=O)CC4)C3)c12 ; CCc1cccc2cccc(N3CCc4c(nc(OCC5(CN(C)C)CC5)nc4N4CCCC(c5ccn(C)n5)C4)C3)c12]

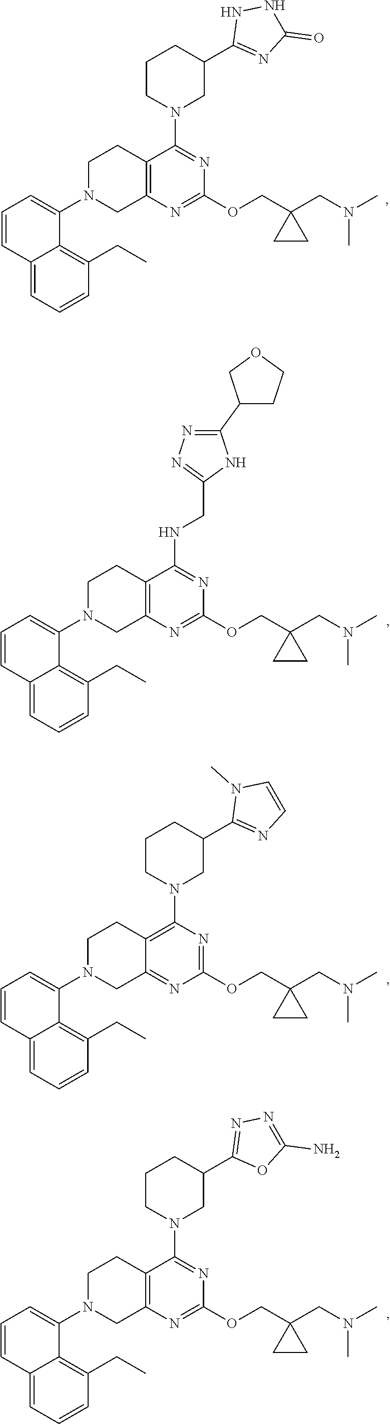 [SMILES: CCc1cccc2cccc(N3CCc4c(nc(OCC5(CN(C)C)CC5)nc4N4CCCC(c5nc(=O)[nH][nH]5)C4)C3)c12 ; CCc1cccc2cccc(N3CCc4c(nc(OCC5(CN(C)C)CC5)nc4NCc4nnc(C5CCOC5)[nH]4)C3)c12 ; CCc1cccc2cccc(N3CCc4c(nc(OCC5(CN(C)C)CC5)nc4N4CCCC(c5nccn5C)C4)C3)c12 ; CCc1cccc2cccc(N3CCc4c(nc(OCC5(CN(C)C)CC5)nc4N4CCCC(c5nnc(N)o5)C4)C3)c12]

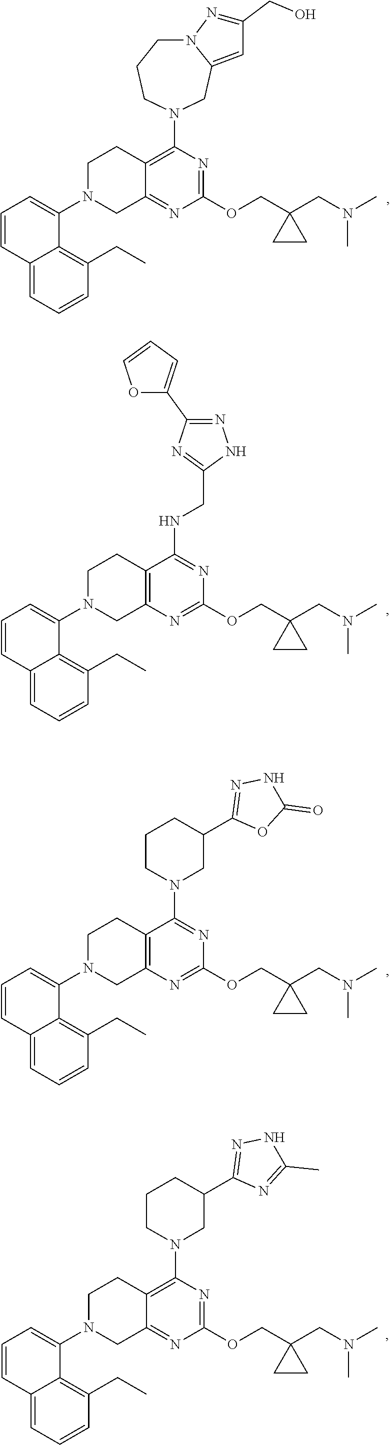 [SMILES: CCc1cccc2cccc(N3CCc4c(nc(OCC5(CN(C)C)CC5)nc4N4CCCn5nc(CO)cc5C4)C3)c12 ; CCc1cccc2cccc(N3CCc4c(nc(OCC5(CN(C)C)CC5)nc4NCc4nc(-c5ccco5)n[nH]4)C3)c12 ; CCc1cccc2cccc(N3CCc4c(nc(OCC5(CN(C)C)CC5)nc4N4CCCC(c5n[nH]c(=O)o5)C4)C3)c12 ; CCc1cccc2cccc(N3CCc4c(nc(OCC5(CN(C)C)CC5)nc4N4CCCC(c5n[nH]c(C)n5)C4)C3)c12]

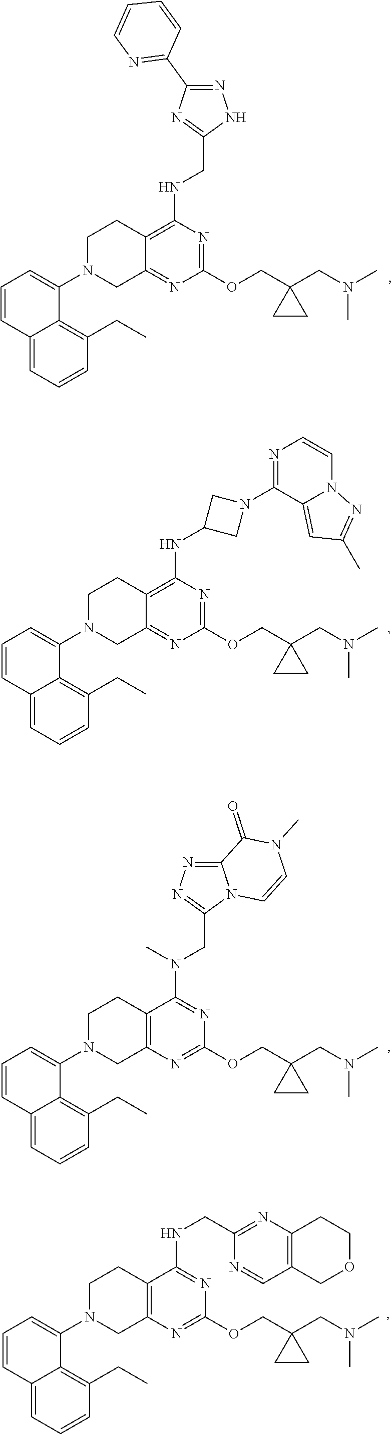 [SMILES: CCc1cccc2cccc(N3CCc4c(nc(OCC5(CN(C)C)CC5)nc4NCc4nc(-c5ccccn5)n[nH]4)C3)c12 ; CCc1cccc2cccc(N3CCc4c(nc(OCC5(CN(C)C)CC5)nc4NC4CN(c5nccn6nc(C)cc56)C4)C3)c12 ; CCc1cccc2cccc(N3CCc4c(nc(OCC5(CN(C)C)CC5)nc4N(C)Cc4nnc5c(=O)n(C)ccn45)C3)c12 ; CCc1cccc2cccc(N3CCc4c(nc(OCC5(CN(C)C)CC5)nc4NCc4ncc5c(n4)CCOC5)C3)c12]

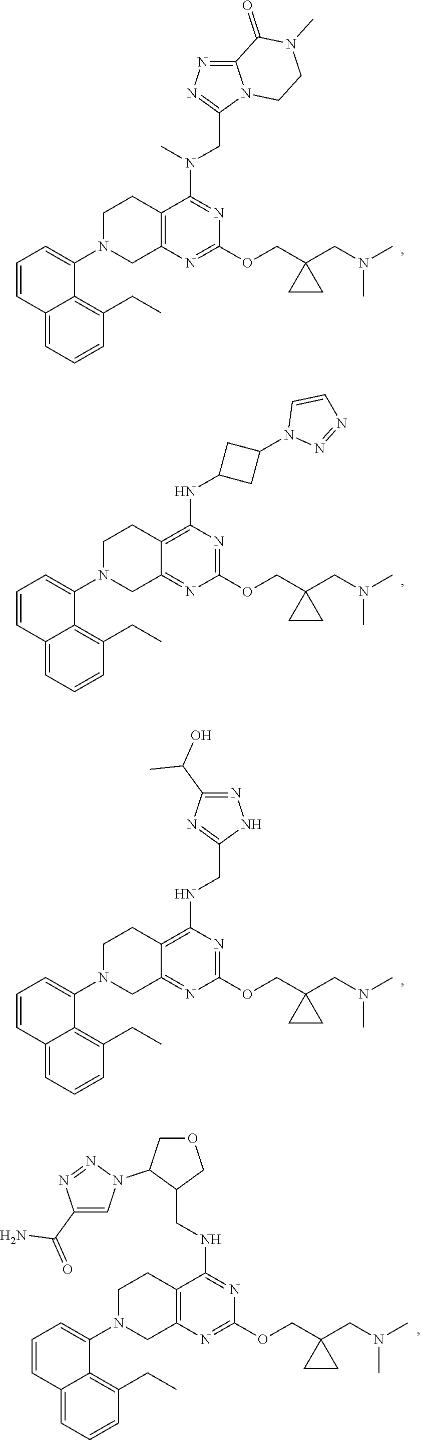 [SMILES: CCc1cccc2cccc(N3CCc4c(nc(OCC5(CN(C)C)CC5)nc4N(C)Cc4nnc5n4CCN(C)C5=O)C3)c12 ; CCc1cccc2cccc(N3CCc4c(nc(OCC5(CN(C)C)CC5)nc4NC4CC(n5ccnn5)C4)C3)c12 ; CCc1cccc2cccc(N3CCc4c(nc(OCC5(CN(C)C)CC5)nc4NCc4nc(C(C)O)n[nH]4)C3)c12 ; CCc1cccc2cccc(N3CCc4c(nc(OCC5(CN(C)C)CC5)nc4NCC4COCC4n4cc(C(N)=O)nn4)C3)c12]

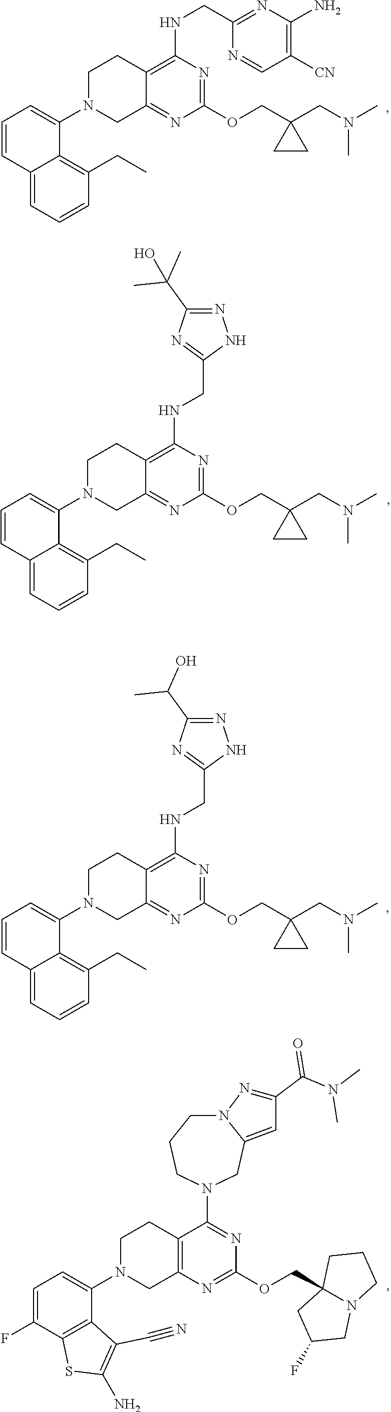 [SMILES: CCc1cccc2cccc(N3CCc4c(nc(OCC5(CN(C)C)CC5)nc4NCc4ncc(C#N)c(N)n4)C3)c12 ; CCc1cccc2cccc(N3CCc4c(nc(OCC5(CN(C)C)CC5)nc4NCc4nc(C(C)(C)O)n[nH]4)C3)c12 ; CCc1cccc2cccc(N3CCc4c(nc(OCC5(CN(C)C)CC5)nc4NCc4nc(C(C)O)n[nH]4)C3)c12 ; CN(C)C(=O)c1cc2n(n1)CCCN(c1nc(OC[C@@]34CCCN3C[C@H](F)C4)nc3c1CCN(c1ccc(F)c4sc(N)c(C#N)c14)C3)C2]

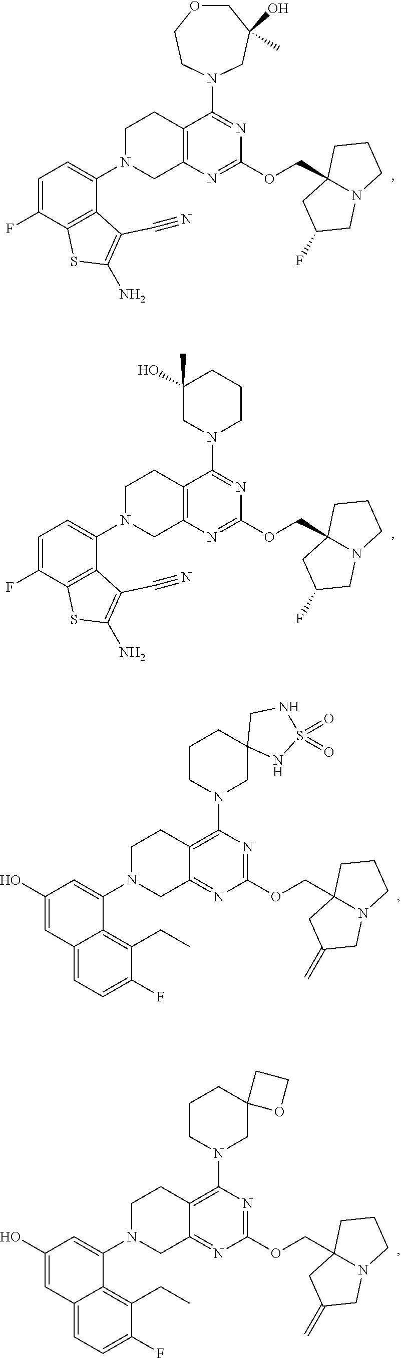 [SMILES: C[C@@]1(O)COCCN(c2nc(OC[C@@]34CCCN3C[C@H](F)C4)nc3c2CCN(c2ccc(F)c4sc(N)c(C#N)c24)C3)C1 ; C[C@@]1(O)CCCN(c2nc(OC[C@@]34CCCN3C[C@H](F)C4)nc3c2CCN(c2ccc(F)c4sc(N)c(C#N)c24)C3)C1 ; C=C1CN2CCCC2(COc2nc3c(c(N4CCCC5(CNS(=O)(=O)N5)C4)n2)CCN(c2cc(O)cc4ccc(F)c(CC)c24)C3)C1 ; C=C1CN2CCCC2(COc2nc3c(c(N4CCCC5(CCO5)C4)n2)CCN(c2cc(O)cc4ccc(F)c(CC)c24)C3)C1]

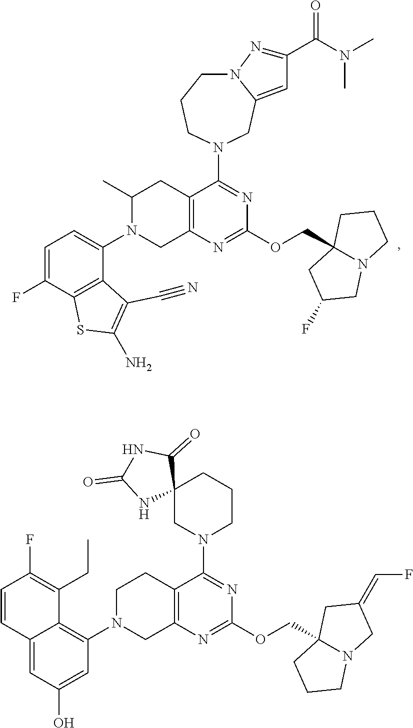 [SMILES: CC1Cc2c(nc(OC[C@@]34CCCN3C[C@H](F)C4)nc2N2CCCn3nc(C(=O)N(C)C)cc3C2)CN1c1ccc(F)c2sc(N)c(C#N)c12 ; CCc1c(F)ccc2cc(O)cc(N3CCc4c(nc(OC[C@@]56CCCN5C/C(=C\F)C6)nc4N4CCC[C@]5(C4)NC(=O)NC5=O)C3)c12]

and pharmaceutically acceptable salts thereof.

In one embodiment, the compounds of Formula (I) include bis-hydrochloride, tris-hydrochloride, trifluoroacetic acid, bis-trifluoroacetic acid, and tris-trifluoracetic acid salts of the above compounds. The compounds of Formula (I) or pharmaceutically acceptable salt thereof may be formulated into pharmaceutical compositions.

### Pharmaceutical Compositions

In another aspect, the invention provides pharmaceutical compositions comprising a KRas wild type, KRas G12A, KRas G12C, KRas G12D, KRas G12R, KRas G12S, KRas G12V, KRas G13D and/or KRas Q61Hinhibitor according to the invention and a pharmaceutically acceptable carrier, excipient, or diluent. Compounds of the invention may be formulated by any method well known in the art and may be prepared for administration by any route, including, without limitation, parenteral, intraperitoneal, intradermal, intracardiac, intraventricular, intracranial, intracerebrospinal, intrasynovial, intrathecal administration, intramuscular injection, intravitreous injection, intravenous injection, intra-arterial injection, oral, buccal, sublingual, transdermal, topical, intranasal, intratracheal, intrarectal, subcutaneous, and topical administration. In certain embodiments, compounds of the invention are administered intravenously in a hospital setting. In one embodiment, administration may be by the oral route. In some embodiments, the provided pharmaceutical compositions may be administered to a subject in need of treatment by injection systemically, such as by intravenous injection; or by injection or application to the relevant site, such as by direct injection via syringe, or direct application to the site when the site is exposed in surgery; or by topical administration.

Parenteral administration can be by bolus injection or continuous infusion. Pharmaceutical compositions for injection may be presented in unit dosage form, e.g., in ampoules or in multi-dose containers, with an added preservative.

The provided pharmaceutical compositions can also be formulated as a depot preparation. Such long-acting formulations may be administered by implantation (for example subcutaneously or intramuscularly) or by intramuscular injection. Thus, for example, the formulations may be modified with suitable polymeric or hydrophobic materials (for example as an emulsion in an acceptable oil) or ion exchange resins, or as sparingly soluble derivatives, for example, as a sparingly soluble salt.

The pharmaceutical compositions may, if desired, be presented in a vial, pack or a medical device, including but not limited to a dispenser device which may contain one or more unit dosage forms containing the active ingredient. In one embodiment the dispenser device can comprise a syringe having a single dose of the liquid formulation ready for injection. The syringe can be accompanied by instructions for administration.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term pharmaceutically acceptable salt refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z-, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. In one embodiment, a dose of the active compound for all of the above-mentioned conditions is in the range from about 0.01 to 300 mg/kg, for example 0.1 to 100 mg/kg per day, and as a further example 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising compounds of the present invention may be used in the methods of use described herein.

### Methods of Use

In yet another aspect, the invention provides for methods for inhibiting wild type KRas, KRas G12A, KRas G12C, KRas G12D, KRas G12R, KRas G12S, KRas G12V and/or KRas Q61H activity in a cell, comprising contacting the cell in which inhibition of wild type KRas or KRas G12A, KRas G12C, KRas G12D, KRas G12R, KRas G12S, KRas G12V and/or Q61H activity is desired with an effective amount of a compound of Formula (I), pharmaceutically acceptable salts thereof, or pharmaceutical compositions containing the compound or pharmaceutically acceptable salt thereof. In one embodiment, the contacting is in vitro. In one embodiment, the contacting is in vivo.

As used herein, the term “contacting” refers to the bringing together of indicated moieties in an in vitro system or an in vivo system. For example, “contacting” wild type KRas, KRas G12A, KRas G12C, KRas G12D, KRas G12R, KRas G12S, KRas G12V, KRas G13D and/or KRas Q61H with a compound provided herein includes the administration of a compound provided herein to an individual or patient, such as a human, having wild type KRas or a KRas G12A, KRas G12C, KRas G12D, KRas G12R, KRas G12S, KRas G12V, KRas G13D and/or KRas Q61H mutation, as well as, for example, introducing a compound provided herein into a sample containing a cellular or purified preparation containing wild type KRas or a KRas G12A, KRas G12C, KRas G12D, KRas G12R, KRas G12S, KRas G12V, KRas G13D or KRas Q61H mutation.

In one embodiment, a cell in which inhibition of wild type KRas, KRas G12A, KRas G12C, KRas G12D, KRas G12R, KRas G12S, KRas G12V, KRas G13D and/or KRas Q61H activity is desired is contacted with an effective amount of a compound of Formula (I) or pharmaceutically acceptable salt thereof to negatively modulate the activity of one or more of wild type KRas, KRas G12A, KRas G12C, KRas G12D, KRas G12R, KRas G12S, KRas G12V, KRas G13D and KRas Q61H.

By negatively modulating the activity of one or more of wild type KRas, KRas G12A, KRas G12C, KRas G12D, KRas G12R, KRas G12S, KRas G12V, KRas G13D and KRas Q61H, the methods described herein are designed to inhibit undesired cellular proliferation resulting from enhanced wild type KRas, KRas G12A, KRas G12C, KRas G12D, KRas G12R, KRas G12S, KRas G12V, KRas G13D and/or KRas Q61H activity within the cell. The cells may be contacted in a single dose or multiple doses in accordance with a particular treatment regimen to affect the desired negative modulation of wild type KRas, KRas G12A, KRas G12C, KRas G12D, KRas G12R, KRas G12S, KRas G12V, KRas G13D and/or KRas Q61H. The ability of compounds to bind one or more of wild type KRas, KRas G12A, KRas G12C, KRas G12D, KRas G12R, KRas G12S, KRas G12V, KRas G13D and KRas Q61H may be monitored in vitro using well known methods, including those described in Examples A and B below. In addition, the inhibitory activity of exemplary compounds in cells may be monitored, for example, by measuring the inhibition of one or more of wild type KRas, KRas G12A, KRas G12C, KRas G12D, KRas G12R, KRas G12S, KRas G12V, KRas G13D and/or KRas Q61H activity of the amount of phosphorylated ERK, for example using the method described in Example C below.

In another aspect, methods of treating cancer in a patient in need thereof, comprising administering to said patient a therapeutically effective amount of a compound of Formula (I) or a pharmaceutically acceptable salt thereof, or a pharmaceutical composition comprising the compound or pharmaceutically acceptable salt thereof are provided.

The compositions and methods provided herein may be used for the treatment of a wild type KRas-associated or KRas G12A, KRas G12C, KRas G12D, KRas G12R, KRas G12S, KRas G12V, KRas G13D and/or KRas Q61H-associated cancer in a patient in need thereof, comprising administering to said patient a therapeutically effective amount of a compound of Formula (I), a pharmaceutically acceptable salt thereof, or a pharmaceutical composition comprising the compound or pharmaceutically acceptable salt thereof are provided. In one embodiment, the wild type KRas-associated or KRas G12A, KRas G12C, KRas G12D, KRas G12R, KRas G12S, KRas G12V, KRas G13D and/or KRas Q61H-associated cancer is lung cancer.

The compositions and methods provided herein may be used for the treatment of a wide variety of cancers including tumors such as lung, prostate, breast, brain, skin, cervical carcinomas, testicular carcinomas, etc. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is non-small cell lung cancer, small cell lung cancer, colorectal cancer, rectal cancer or pancreatic cancer. In certain embodiments, the cancer is non-small cell lung cancer.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

Also provided herein is a compound of Formula (I) or a pharmaceutically acceptable salt thereof, or a pharmaceutical composition thereof as defined herein for use in therapy.

Also provided herein is a compound of Formula (I) or a pharmaceutically acceptable salt thereof, or a pharmaceutical composition thereof as defined herein for use in the treatment of cancer.

Also provided herein is a compound of Formula (I) or a pharmaceutically acceptable salt thereof, or a pharmaceutical composition thereof for use in the inhibition of wild type KRas or KRas G12A, KRas G12C, KRas G12D, KRas G12R, KRas G12S, KRas G12V, KRas G13D and/or KRas Q61H.

Also provided herein is a compound of Formula (I) or a pharmaceutically acceptable salt thereof, or a pharmaceutical composition thereof as defined herein, for use in the treatment of a wild type KRas-associated or a KRas G12A, KRas G12C, KRas G12D, KRas G12R, KRas G12S, KRas G12V, KRas G13D and/or KRas Q61H-associated disease or disorder.

Also provided herein is the use of a compound of Formula (I) or a pharmaceutically acceptable salt thereof, as defined herein in the manufacture of a medicament for the treatment of cancer.

Also provided herein is a use of a compound of Formula (I) or a pharmaceutically acceptable salt thereof, as defined herein in the manufacture of a medicament for the inhibition of activity of wild type KRas, KRas G12A, KRas G12C, KRas G12D, KRas G12R, KRas G12S, KRas G12V, KRas G13D and/or KRas Q61H.

Also provided herein is the use of a compound of Formula (I) or a pharmaceutically acceptable salt thereof, as defined herein, in the manufacture of a medicament for the treatment of a wild type KRas-associated or KRas G12A, KRas G12C, KRas G12D, KRas G12R, KRas G12S, KRas G12V, KRas G13D and/or KRas Q61H-associated disease or disorder.

Also provided herein is a method for treating cancer in a patient in need thereof, the method comprising (a) determining that cancer is associated with wild type KRas or a KRas G12A, KRas G12C, KRas G12D, KRas G12R, KRas G12S, KRas G12V, KRas G13D and/or KRas Q61H mutation (e.g., as determined using a regulatory agency-approved, e.g., FDA-approved, assay or kit); and (b) administering to the patient a therapeutically effective amount of a compound of Formula (I), or a pharmaceutically acceptable salt thereof, or a pharmaceutical composition thereof.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

### REACTION SCHEMES AND EXAMPLES

The compounds of the present invention may be prepared from commercially available reagents using the synthetic methods and reaction schemes described herein, or using other reagents and conventional methods well known to those skilled in the art. For instance, compounds of the present invention may be prepared according to the reaction schemes and examples outlines below.

The compounds of the present invention may have one or more chiral center and may be synthesized as stereoisomeric mixtures, isomers of identical constitution that differ in the arrangement of their atoms in space. The compounds may be used as mixtures or the individual components/isomers may be separated using commercially available reagents and conventional methods for isolation of stereoisomers and enantiomers well-known to those skilled in the art, e.g., using CHIRALPAK® (Sigma-Aldrich) or CHIRALCEL® (Diacel Corp) chiral chromatographic HPLC columns according to the manufacturer's instructions. Alternatively, compounds of the present invention may be synthesized using optically pure and chiral reagents to prepare individual isomers or enantiomers. Unless otherwise indicated, all chiral (enantiomeric and diastereomeric) and racemic forms are within the scope of the invention. Unless otherwise indicated, whenever the specification, including the claims, refers to compounds of the invention, the term “compound” is to be understood to encompass all chiral (enantiomeric and diastereomeric) and racemic forms.

The compounds of the present invention may be in anhydrous, solvated or hydrated forms, and all such forms are included within the scope of the invention.

### Example 233

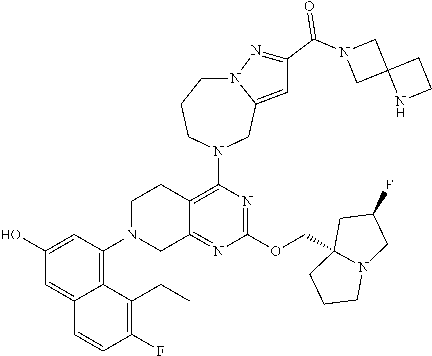 [SMILES: CCc1c(F)ccc2cc(O)cc(N3CCc4c(nc(OC[C@@]56CCCN5C[C@H](F)C6)nc4N4CCCn5nc(C(=O)N6CC7(CCN7)C6)cc5C4)C3)c12]

### (5-(7-(8-ethyl-7-fluoro-3-hydroxynaphthalen-1-yl)-2-(((2R,7aS)-2-fluorohexahydro-1 H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(1,6-diazaspiro[3.3]heptan-6-yl)methanone

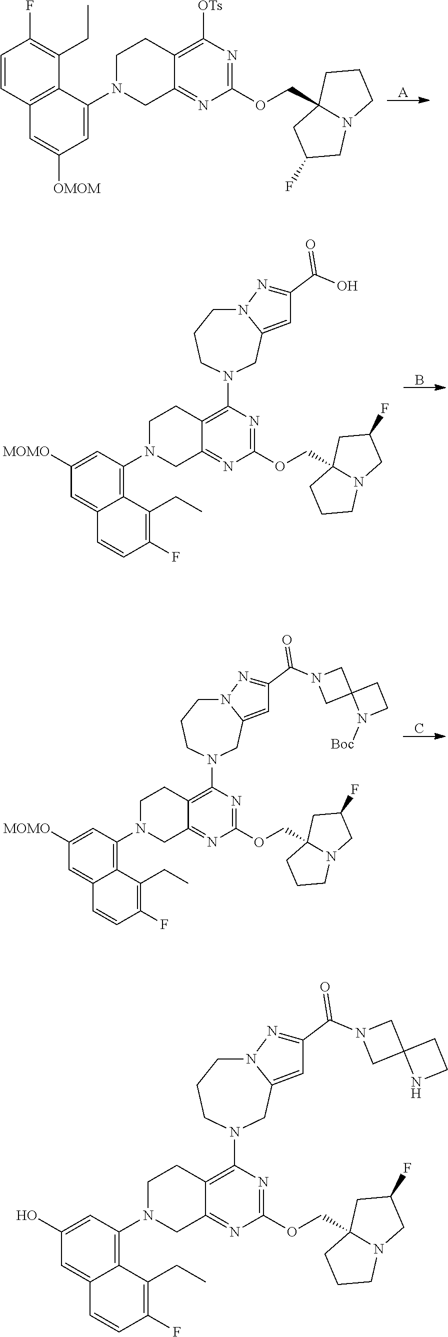

Step A. 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-5,6,7,8-tetrahydro-4H-pyrazolo[1,5-a][1,4]diazepine-2-carboxylic acid: To a mixture of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl-4-methylbenzenesulfonate (150 mg, 1.0 equiv) and 5,6,7,8-tetrahydro-4H-pyrazolo[1,5-a][1,4]diazepine-2-carboxylic acid (50.0 mg, 1.1 equiv, HCl salt) in DMF (1 mL) were added 4A molecular sieve (30 mg, 1.0 equiv) and N,N-diethylpropan-2-amine (279 mg, 10 equiv). The reaction was stirred at 40° C. for 12 hours. The mixture was filtered and purified by reversed phase flash chromatography [water (FA, 0.1%)/acetonitrile] to afford the title compound (100 mg, 65% yield) as yellow solid; LCMS (ESI, M+1): m/z=704.4

Step B. tert-butyl 6-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepine-2-carbonyl)-1,6-diazaspiro[3.3]heptane-1-carboxylate: To a mixture of 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepine-2-carboxylic acid (70.0 mg, 1.0 equiv) in DMF (0.5 mL) were added HATU (75.6 mg, 2.0 equiv), N,N-diethylpropan-2-amine (38.6 mg, 3.0 equiv) and tert-butyl 1,6-diazaspiro[3.3]heptane-1-carboxylate (49.3 mg, 2.5 equiv). The reaction was stirred at 20° C. for 0.5 hours. The mixture was filtered and purified by prep-HPLC [Welch Ultimate C18 150×25 mm×5 pm; A: water (FA), B: ACN; B %: 29%-59% over 30 min] and lyophilized to afford the title compound (52 mg, 58% yield) as yellow solid.

Step C. (5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(1,6-diazaspiro[3.3]heptan-6-yl)methanone: To a mixture of tert-butyl 6-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepine-2-carbonyl)-1,6-diazaspiro[3.3]heptane-1-carboxylate (34.0 mg, 1.0 equiv) in DCM (0.5 mL) was added TFA (770 mg, 175 equiv). The reaction was stirred at 0° C. for 1 hour. The mixture was concentrated, dissolved in methanol (3.0 mL), neutralized with solid NaHCO₃, filtered, and purified by prep-HPLC [Phenomenex luna 150×25 mm×10 μm; A: water (FA), B: ACN; B %-: 7%-37% over 9 min] and lyophilized to afford the title compound (1.91 mg, 5.7% yield, FA[Formic acid] salt) as yellow solid. 1H NMR (400 MHz, dimethylsulfoxide-d6+deuterium oxide-d2) δ=7.57 (dd, J=6.0, 9.2 Hz, 1H), 7.32-7.15 (m, 1H), 6.98 (s, 2H), 6.58 (s, 1H), 5.35-5.13 (m, 1H), 5.03-4.92 (m, 1H), 4.77-4.67 (m, 1H), 4.63-4.38 (m, 4H), 4.25-4.03 (m, 3H), 3.98-3.84 (m, 2H), 3.55 (br d, J=17.2 Hz, 1H), 3.48-3.38 (m, 3H), 3.34-3.16 (m, 3H), 3.15-2.93 (m, 5H), 2.88-2.73 (m, 1H), 2.65-2.57 (m, 1H), 2.45-2.36 (m, 1H), 2.22-1.96 (m, 4H), 1.96-1.63 (m, 6H), 1.07-0.99 (m, 3H); 19F NMR (400 MHz, dimethylsulfoxide-d6+deuterium oxide-d2) 6=-121.055, -171.774; LCMS (ESI, M+1): m/z=740.5.

### Example 234

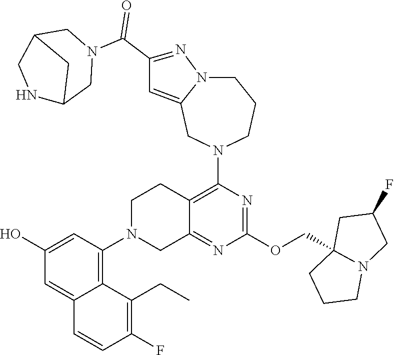 [SMILES: CCc1c(F)ccc2cc(O)cc(N3CCc4c(nc(OC[C@@]56CCCN5C[C@H](F)C6)nc4N4CCCn5nc(C(=O)N6CC7CNC(C7)C6)cc5C4)C3)c12]

### 3,6-diazabicyclo[3.2.1]octan-3-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

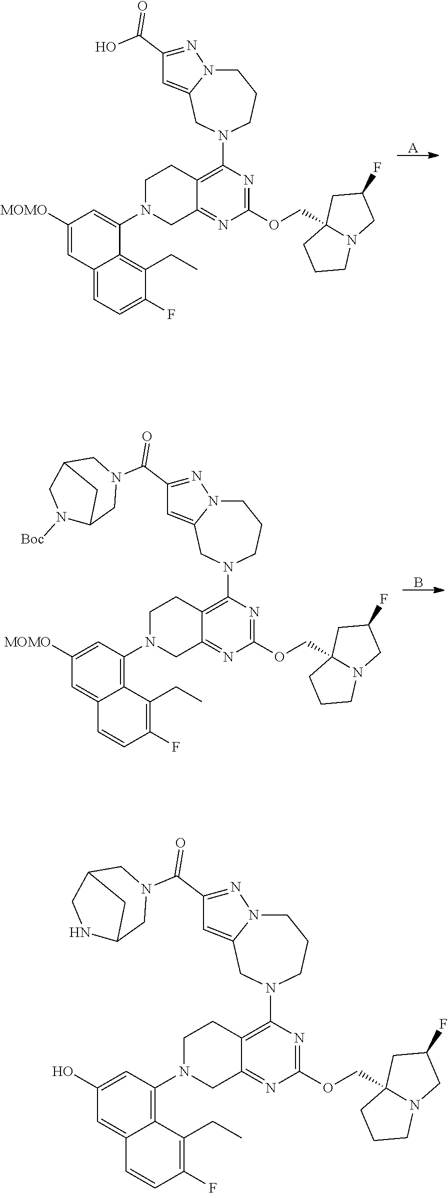

Synthesized according to Example 233 except that HCl instead of TFA was used in the last step. The title compound was obtained as orange solid (FA salt). 1H NMR (400 MHz, dimethylsulfoxide-d6+deuterium oxide-d2) δ=7.56 (dd, J=6.0, 8.8 Hz, 1H), 7.22 (t, J=9.6 Hz, 1H), 7.05-6.89 (m, 2H), 6.61-6.44 (m, 1H), 5.33-5.12 (m, 1H), 5.08-4.88 (m, 1H), 4.81-4.65 (m, 1H), 4.61-4.40 (m, 3H), 4.35-4.07 (m, 2H), 4.00-3.87 (m, 2H), 3.76-3.65 (m, 1H), 3.62-3.50 (m, 1H), 3.41 (br d, J=7.6 Hz, 1H), 3.14 (br s, 2H), 3.13-3.00 (m, 5H), 2.99-2.82 (m, 3H), 2.81-2.72 (m, 1H), 2.69-2.60 (m, 1H), 2.55 (br d, J=2.0 Hz, 1H), 2.21-2.10 (m, 1H), 2.09-1.87 (m, 3H), 1.86-1.75 (m, 5H), 1.74-1.61 (m, 2H), 1.09-0.99 (m, 3H); 19F NMR (400 MHz, dimethylsulfoxide-d6+deuterium oxide-d2) δ=−121.048, -171.624; LCMS (ESI, M+1): m/z=754.6.

### Example 235

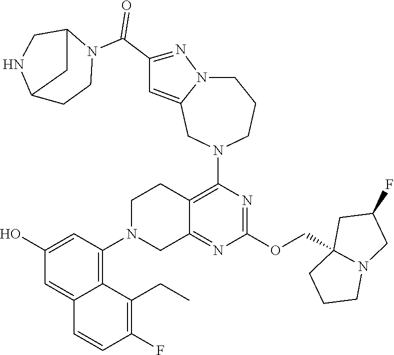 [SMILES: CCc1c(F)ccc2cc(O)cc(N3CCc4c(nc(OC[C@@]56CCCN5C[C@H](F)C6)nc4N4CCCn5nc(C(=O)N6CCC7CC6CN7)cc5C4)C3)c12]

2,6-diazabicyclo[3.2.1]octan-2-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7, 8-tetrahydro-4H-pyrazolo[1, 5-a][1,4]diazepin-2-yl)methanone

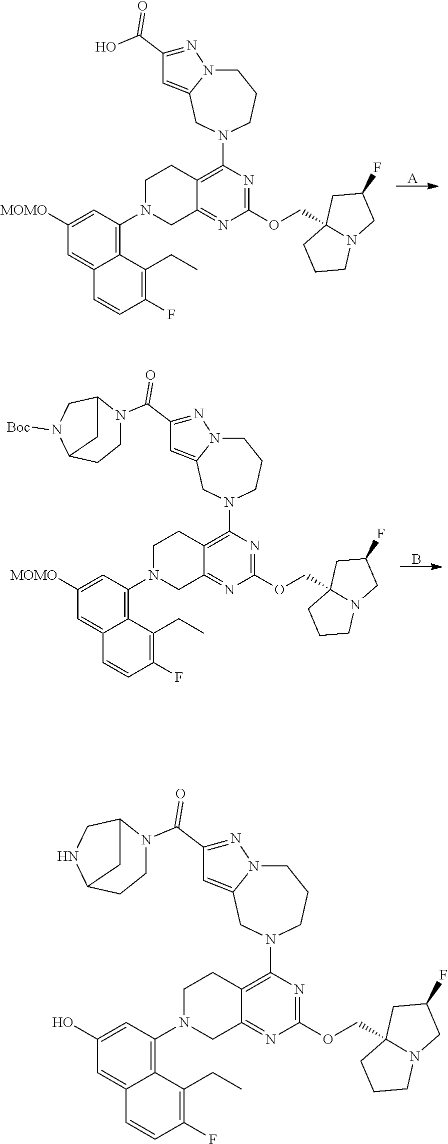

Synthesized according to Example 233 except that HCl instead of TFA was used in the last step. The title compound was obtained as yellow solid (FA salt). 1H NMR (400 MHz, dimethylsulfoxide-d6+D20-d2) δ=7.61-7.51 (m, 1H), 7.28-7.18 (m, 1H), 7.03-6.92 (m, 2H), 6.63-6.53 (m, 1H), 5.22 (br d, J=13.8 Hz, 2H), 5.04-4.93 (m, 1H), 4.80-4.68 (m, 1H), 4.65-4.15 (m, 4H), 4.13-4.05 (m, 2H), 3.97-3.90 (m, 1H), 3.87-3.77 (m, 2H), 3.60-3.52 (m, 1H), 3.52-3.29 (m, 4H), 3.28-3.09 (m, 6H), 3.08-2.98 (m, 1H), 2.91 (br s, 1H), 2.63 (br s, 1H), 2.23-2.07 (m, 3H), 2.05-1.80 (m, 7H), 1.78-1.66 (m, 2H), 1.07-0.95 (m, 3H); 19F NMR (400 MHz, dimethylsulfoxide-d6+D20-d2) δ=−120.845, -171.812; LCMS (ESI, M+1): m/z=754.6.

### Example 236

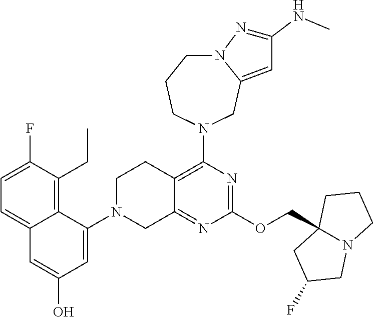 [SMILES: CCc1c(F)ccc2cc(O)cc(N3CCc4c(nc(OC[C@@]56CCCN5C[C@H](F)C6)nc4N4CCCn5nc(NC)cc5C4)C3)c12]

### 5-ethyl-6-fluoro-4-(2-(((2R,7aS)-2-fluorohexahydro-1H-pyrrolizin-7a-yl)methoxy)-4-(2-(methylamino)-7,8-dihydro-4H-pyrazolo[1,5-a][1,4]diazepin-5(6H)-yl)-5,6-dihydropyrido[3,4-d]pyrimidin-7(8H)-yl)naphthalen-2-ol

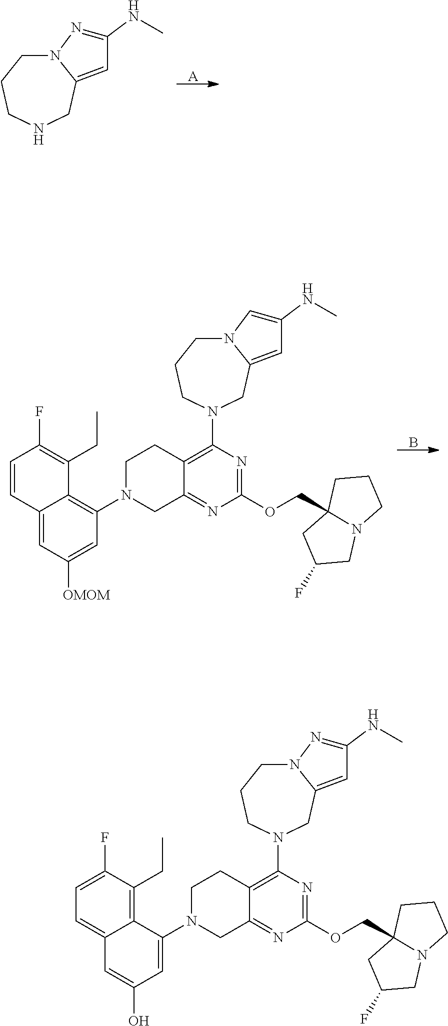

Step A. 5-(7-(8-ethyl-7-fluoro-3-(methoxymethoxy)naphthalen-1-yl)-2-(((2R,7aS)-2-fluorotetrahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepin-2-amine: To a solution of 7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (130 mg, 1 equiv) and N-methyl-5,6,7,8-tetrahydro-4H-pyrazolo[1,5-a][1,4]diazepin-2-amine (60 mg, 1.93 equiv) in DMF (0.65 mL) were added N,N-diethylpropan-2-amine (48.3 mg, 2 equiv) and 4A molecular sieve (20 mg). The mixture was stirred at 40° C. for 24 hours. The reaction was filtered and purified with reversed phase flash chromatography [water (FA, 0.1%)/acetonitrile=3/2] to afford the title compound (100 mg, 76.81% yield) as black-brown solid; LCMS (ESI, M+1): m/z=689.5.

Step B. 5-ethyl-6-fluoro-4-(2-(((2R,7aS)-2-fluorohexahydro-1H-pyrrolizin-7a-yl)methoxy)-4-(2-(methylamino)-7,8-dihydro-4H-pyrazolo[1,5-a][1,4]diazepin-5(6H)-yl)-5,6-dihydropyrido[3,4-d]pyrimidin-7(8H)-yl)naphthalen-2-ol: To a solution of 5-(7-(8-ethyl-7-fluoro-3-(methoxymethoxy)naphthalen-1-yl)-2-(((2R,7aS)-2-fluorotetrahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepin-2-amine (100 mg, 1 equiv) in MeOH (1 mL) was added HCl/MeOH (4 M, 1 mL). The mixture was stirred at 0° C. for 2 hours. The reaction was concentrated under reduced pressure to give a residue. The mixture was diluted with H₂O (10 mL) and extracted with ethyl acetate (5 mL×4). The combined organic layers were dried over anhydrous Na₂SO₄, concentrated, and purified with prep-HPLC [column: Phenomenex luna C18 150×25 mm×10 μm; mobile phase: water(FA)-ACN; B %: 15%-45%, 2 minutes] and [column: Waters Xbridge 150×25 mm×5 μm; mobile phase: water(NH₄HCO₃)-ACN; B %: 40%-70%, 9 minutes] to afford the title compound (18.89 mg, 19.37% yield) as yellow solid; 1H NMR (400 MHz, METHANOL-d4) δ=7.51 (dd, J=5.6, 9.2 Hz, 1H), 7.14 (t, J=9.4 Hz, 1H), 6.96 (s, 2H), 5.54 (d, J=2.8 Hz, 1H), 5.35-5.14 (m, 1H), 4.83 (s, 1H), 4.66 (br d, J=16.4 Hz, 1H), 4.24 (br d, J=4.4 Hz, 2H), 4.17-3.98 (m, 4H), 3.96-3.83 (m, 1H), 3.68 (dd, J=2.0, 17.6 Hz, 1H), 3.55-3.34 (m, 3H), 3.25-3.10 (m, 5H), 2.98 (dt, J=5.6, 9.6 Hz, 1H), 2.74 (s, 3H), 2.72-2.65 (m, 1H), 2.32-1.77 (m, 8H), 1.12 (t, J=7.2 Hz, 3H); LCMS (ESI, M+1): m/z=645.4.

### Example 237

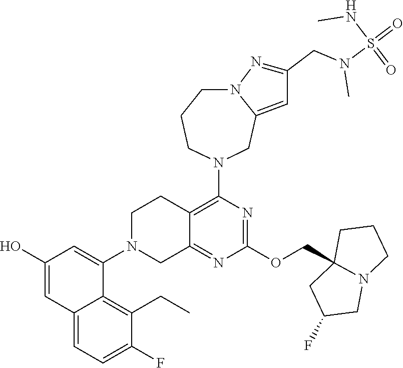 [SMILES: CCc1c(F)ccc2cc(O)cc(N3CCc4c(nc(OC[C@@]56CCCN5C[C@H](F)C6)nc4N4CCCn5nc(CN(C)S(=O)(=O)NC)cc5C4)C3)c12]

### 7-(8-ethyl-7-fluoro-3-hydroxy-1-naphthyl)-2-(((2R,7aS)-2-fluorotetrahydro-1H-pyrrolizin-7a(5H)-yl)methoxy)-4-(2-((methylsulfamoyl)methylamino)methyl-7,8-dihydro-4H-pyrazolo[1,5-a][1,4]diazepin-5(6H)-yl)-6,8-dihydro-5H-pyrido[3,4-d]pyrimidine

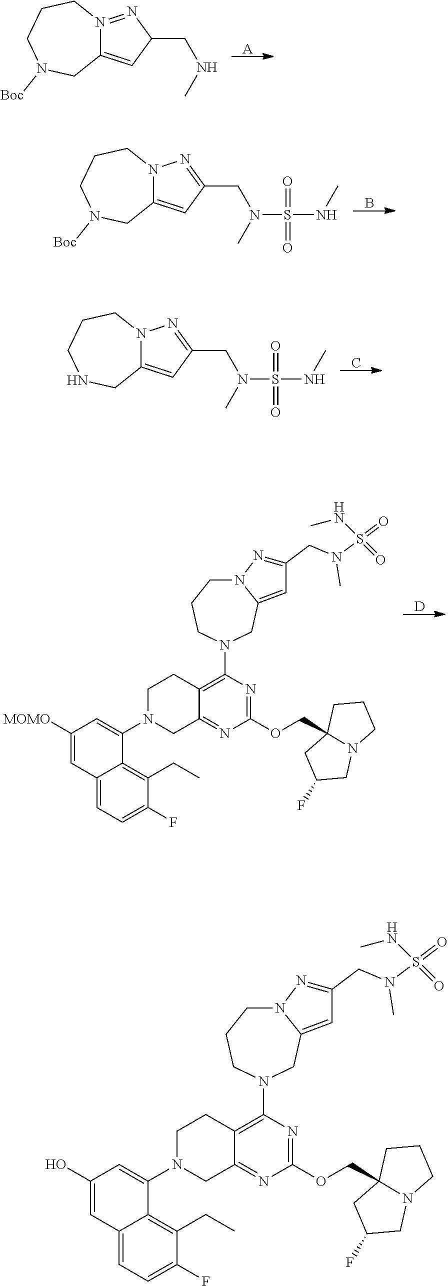

Step A. tert-butyl 2-((methyl(N-methylsulfamoyl)amino)methyl)-7,8-dihydro-4H-pyrazolo[1,5-a][1,4]diazepine-5(6H)-carboxylate: To a mixture of tert-butyl 2-((methylamino)methyl)-7,8-dihydro-4H-pyrazolo[1,5-a][1,4]diazepine-5(6H)-carboxylate (200 mg, 1.0 equiv) and TEA (217 mg, 3.0 equiv) in DCM (2.0 mL) was added methylsulfamoyl chloride (92.4 mg, 1.0 equiv). The reaction was stirred at 20° C. for 2 hours. The mixture was concentrated and purified by reversed phase flash chromatography (0.1% FA condition) to afford the title compound (120 mg, 45% yield) as white oil; LCMS (ESI, M+1): m/z=374.1.

Step B. 2-[[methyl(methylsulfamoyl)amino]methyl]-5,6,7,8-tetrahydro-4H-pyrazolo[1,5-a][1,4]diazepine: A mixture of tert-butyl 2-((methyl(N-methylsulfamoyl)amino)methyl)-7,8-dihydro-4H-pyrazolo[1,5-a][1,4]diazepine-5(6H)-carboxylate (90.0 mg, 1.0 equiv) in HCl-MeOH (4 M, 1.5 mL, 25 equiv) was stirred at 20° C. for 1 hour. The mixture was concentrated under vacuum to afford the title compound (110 mg, crude) as white solid and was used into next step without further purification.

Step C. 7-(8-ethyl-7-fluoro-3-(methoxymethoxy)-1-naphthyl)-2-(((2R,7aS)-2-fluorotetrahydro-1H-pyrrolizin-7a(5H)-yl)methoxy)-4-(2-((methylsulfamoyl)methylamino)methyl-7,8-dihydro-4H-pyrazolo[1,5-a][1,4]diazepin-5(6H)-yl)-6,8-dihydro-5H-pyrido[3,4-d]pyrimidine: To a mixture of 7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (115 mg, 1.0 equiv) and 2-[[methyl(methylsulfamoyl)amino]methyl]-5,6,7,8-tetrahydro-4H-pyrazolo[1,5-a][1,4]diazepine (90.0 mg, 2.0 equiv) in DMF (1.0 mL) were added N,N-diethylpropan-2-amine (213 mg, 10 equiv) and 4A molecular sieve (10.0 mg). The reaction was stirred at 90° C. for 7 hours. The reaction was filtered and purified by reversed phase flash chromatography (C18, 0.1% FA condition) to afford the title compound (80.0 mg, 36% yield) as yellow solid; LCMS (ESI, M+1): m/z=796.5.

Step D. 7-(8-ethyl-7-fluoro-3-hydroxy-1-naphthyl)-2-(((2R,7aS)-2-fluorotetrahydro-1H-pyrrolizin-7a(5H)-yl)methoxy)-4-(2-((methylsulfamoyl)methylamino)methyl-7,8-dihydro-4H-pyrazolo[1,5-a][1,4]diazepin-5(6H)-yl)-6,8-dihydro-5H-pyrido[3,4-d]pyrimidine: A mixture of 7-(8-ethyl-7-fluoro-3-(methoxymethoxy)-1-naphthyl)-2-(((2R,7aS)-2-fluorotetrahydro-1H-pyrrolizin-7a(5H)-yl)methoxy)-4-(2-((methylsulfamoyl)methylamino)methyl-7,8-dihydro-4H-pyrazolo[1,5-a][1,4]diazepin-5(6H)-yl)-6,8-dihydro-5H-pyrido[3,4-d]pyrimidine (70.0 mg, 1.0 equiv) in HCl-MeOH (4 M, 1.5 mL, 68 equiv) was stirred at 20° C. for 0.5 hours. The mixture was concentrated and basified by saturated NaHCO₃ solution to pH-7 and extracted with ethyl acetate (2×3 mL). The combined organic layers were dried over Na₂SO₄, concentrated under reduced pressure, and purified by prep-HPLC (column: Phenomenex luna C18 15×25 mm×10 μm; mobile phase: [water (FA)-ACN]; B %: 15%-45%, 10 min) to afford the title compound (20.5 mg, 30% yield, FA salt) as orange solid. 1H NMR (400 MHz, METHANOL-d4) δ=7.51 (dd, J=5.6, 8.8 Hz, 1H), 7.15 (t, J=9.6 Hz, 1H), 6.97 (s, 2H), 6.31 (s, 1H), 5.46-5.25 (m, 1H), 5.05-4.94 (m, 2H), 4.46 (br s, 2H), 4.30-4.12 (m, 5H), 4.06 (br d, J=16.8 Hz, 2H), 3.72-3.63 (m, 1H), 3.57-3.50 (m, 1H), 3.39 (br d, J=8.4 Hz, 5H), 3.29-3.09 (m, 3H), 2.79-2.68 (m, 4H), 2.60 (s, 3H), 2.44-2.14 (m, 4H), 2.13-1.92 (m, 4H), 1.12 (br t, J=7.2 Hz, 3H); LCMS (ESI, M+1): m/z=752.6.

### Example 238

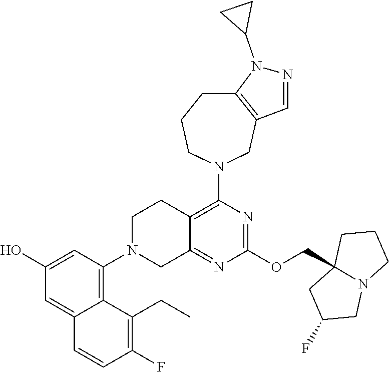 [SMILES: CCc1c(F)ccc2cc(O)cc(N3CCc4c(nc(OC[C@@]56CCCN5C[C@H](F)C6)nc4N4CCCc5c(cnn5C5CC5)C4)C3)c12]

### 4-(4-(1-cyclopropyl-7,8-dihydropyrazolo[4,3-c]azepin-5(1H,4H,6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

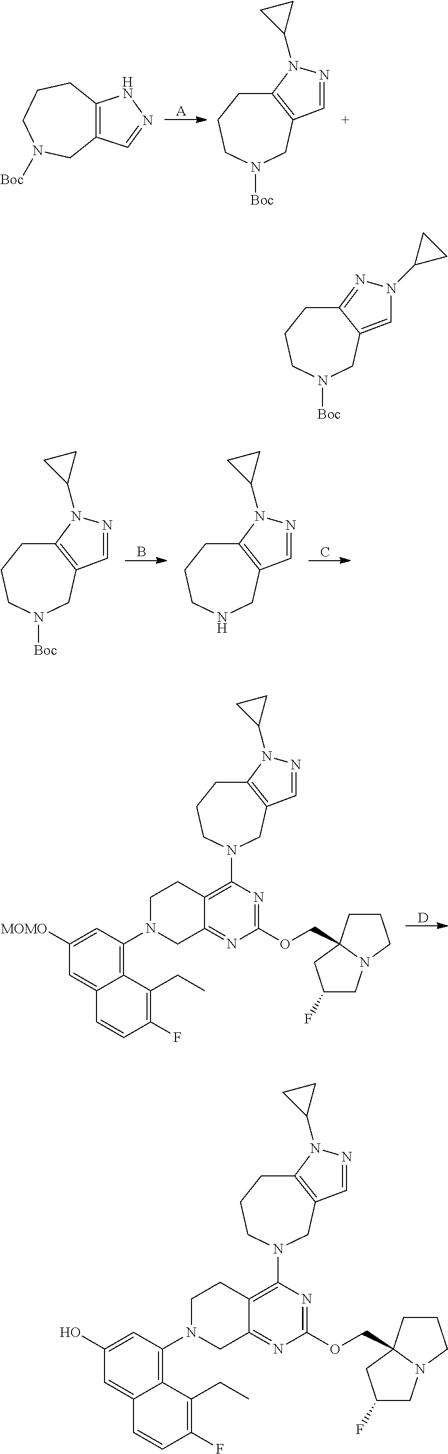

Step A. tert-butyl 2-cyclopropyl-4,6,7,8-tetrahydropyrazolo[4,3-c]azepine-5(2H)-carboxylate: To a mixture of tert-butyl 4,6,7,8-tetrahydro-1H-pyrazolo[4,3-c]azepine-5-carboxylate (800 mg, 1.0 equiv), cyclopropylboronic acid (579 mg, 2.0 equiv), 4A molecular sieve (400 mg), Na₂CO₃ (715 mg, 2 equiv) and 2-(2-pyridyl)pyridine (526 mg, 1 equiv) in DCE (8 mL) was added Cu(OAc)₂ (612 mg, 1.0 equiv). The reaction was degassed and purged with 02 3 times and stirred at 100° C. for 3 hours under 02 atmosphere. The mixture was added into ice-water (10 mL), extracted with ethyl acetate (3×30 mL), washed with brine (50 mL), dried over Na₂SO₄, concentrated, and purified with prep-HPLC (YMC Triart C18 150×25 mm x Sum; A: water[(FA)-ACN]; B: ACN, B %: 35%-65%, over 10 min) followed by SFC separation (DAICEL CHIRALPAK IC(250 mm×30 mm, 5 um); A: [0.1% NH₃H₂O MeOH];B: ACN, B %: 25%-25%,4.5 min) to afford two regioisomers, tert-butyl 1-cyclopropyl-4,6,7,8-tetrahydropyrazolo[4,3-c]azepine-5(1H)-carboxylate (400 mg, 23% yield) as colorless oil; 1H NMR (400 MHz, CHLOROFORM-d) δ=7.27-7.07 (m, 1H), 4.42-4.22 (m, 2H), 3.71-3.58 (m, 2H), 3.31-3.26 (m, 1H), 3.01-2.93 (m, 2H), 1.89 (br s, 2H), 1.48-1.36 (m, 9H), 1.14-1.08 (m, 2H), 1.07-1.00 (m, 2H); LCMS (ESI, M+1): m/z=:278.2 tert-butyl 2-cyclopropyl-4,6,7,8-tetrahydropyrazolo[4,3-c]azepine-5(2H)-carboxylate (420 mg, 29% yield) as colorless oil; 1H NMR (400 MHz, CHLOROFORM-d) δ=7.31-7.08 (m, 1H), 4.36-4.17 (m, 2H), 3.63 (br s, 2H), 3.47 (br s, 1H), 2.94-2.80 (m, 2H), 1.79 (br s, 2H), 1.42 (br s, 9H), 1.09-1.01 (m, 2H), 1.00-0.92 (m, 2H); LCMS (ESI, M+1): m/z=:278.2.

Step B. 1-cyclopropyl-1,4,5,6,7,8-hexahydropyrazolo[4,3-c]azepine: To a solution of tert-butyl 1-cyclopropyl-4,6,7,8-tetrahydropyrazolo[4,3-c]azepine-5(1H)-carboxylate (100 mg, 1.0 equiv) in DCM (1 mL) was added TFA (1.54 g, 37 equiv). The reaction was stirred at 0-25° C. for 10 minutes under N2 atmosphere. The mixture was diluted with saturated ammonium chloride solution (20 mL) and extracted with ethyl acetate (3×30 mL). The combined organic layers were washed with brine (20 mL), dried over sodium sulfate, and concentrated to afford the title compound (60.0 mg, 94% yield) as yellow solid; LCMS (ESI, M+1): m/z=178.1.

Step C. 1-cyclopropyl-5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,4,5,6,7,8-hexahydropyrazolo[4,3-c]azepine: To a mixture of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (150 mg, 1.0 equiv), N,N-diethylpropan-2-amine (41.8 mg, 1.5 equiv) and 4A molecular sieve (30 mg, 1.0 equiv) in DMF (0.3 mL) was added 1-cyclopropyl-1,4,5,6,7,8-hexahydropyrazolo[4,3-c]azepine (60 mg, 1.57 equiv). The reaction was degassed and purged with N2 3 times and stirred at 40° C. for 12 hours under N2 atmosphere. The mixture was diluted with water (40 mL) and extracted with ethyl acetate (2×30 mL). The combined layers were washed with brine (40 mL), dried over sodium sulfate, concentrated, and purified by reversed phase column (C 18, A: water[(0.1% FA)-ACN]; B %: 45%-65%, over 25 min) to afford the title compound (140 mg, 93% yield) as yellow oil; LCMS (ESI, M+1): m/z=700.2.

Step D. 4-(4-(1-cyclopropyl-7,8-dihydropyrazolo[4,3-c]azepin-5(1H,4H,6H)-yl)-2-(((2R,7aS)-2-fluorohexahydro-1H-pyrrolizin-7a-yl)methoxy)-5,6-dihydropyrido[3,4-d] pyrimidin-7(8H)-yl)-5-ethyl-6-fluoronaphthalen-2-ol: To a solution of 1-cyclopropyl-5-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,4,5,6,7,8-hexahydropyrazolo[4,3-c]azepine (100 mg, 1.0 equiv) in MeOH (0.7 mL) was added HCl-MeOH (4 M, 350 μL 10 equiv). The reaction was stirred at 25° C. for 0.5 hours. The mixture was concentrated and purified by reversed phase column (column: Phenomenex luna C18 150×25 mm×10 um; A: water[(FA)-ACN];B %: 18%-48%, over 2 min) to afford the title compound (140 mg, 93% yield, FA salt) as light yellow solid. 1H NMR (400 MHz, METHANOL-d4) δ=8.49 (s, 1H), 7.50 (dd, J=6.0, 8.8 Hz, 1H), 7.35 (d, J=2.8 Hz, 1H), 7.24-7.06 (m, 1H), 6.96 (s, 2H), 5.57-5.27 (m, 1H), 4.83-4.58 (m, 2H), 4.38-4.21 (m, 2H), 4.20-3.96 (m, 3H), 3.68-3.46 (m, 5H), 3.43-3.33 (m, 3H), 3.24 (dt, J=5.5, 9.9 Hz, 2H), 3.18-3.09 (m, 2H), 3.08-2.97 (m, 1H), 2.73 (br d, J=14.8 Hz, 1H), 2.55-2.34 (m, 2H), 2.33-2.22 (m, 1H), 2.22-2.10 (m, 3H), 2.08-1.86 (m, 2H), 1.16-0.96 (m, 7H). LCMS (ESI, M+1): m/z=656.3

### Example 239

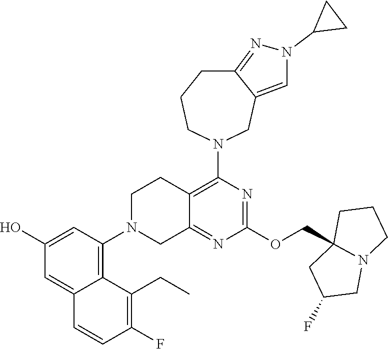 [SMILES: CCc1c(F)ccc2cc(O)cc(N3CCc4c(nc(OC[C@@]56CCCN5C[C@H](F)C6)nc4N4CCCc5nn(C6CC6)cc5C4)C3)c12]

### 4-(4-(2-cyclopropyl-7,8-dihydropyrazolo[4,3-c]azepin-5(2H,4H,6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

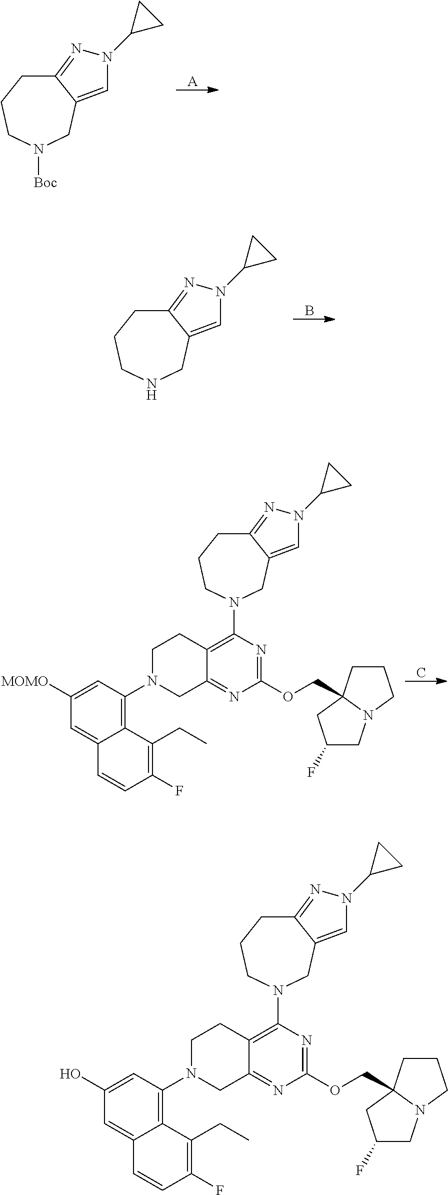

Step A. 2-cyclopropyl-2,4,5,6,7,8-hexahydropyrazolo[4,3-c]azepine: To a solution of tert-butyl 2-cyclopropyl-4,6,7,8-tetrahydropyrazolo[4,3-c]azepine-5(2H)-carboxylate (100 mg, 1.0 equiv), in DCM (1 mL) was added TFA (1.54 g, 37 equiv). The reaction was stirred at 0-25° C. for 10 minutes under N2 atmosphere. The mixture was diluted with saturated NaHCO₃ (20 mL) and extracted with ethyl acetate (3×30 mL). The combined organic layers were washed with brine (20 mL), dried over sodium sulfate, and concentrated to afford the title compound (60.0 mg, 94% yield) as yellow solid; LCMS (ESI, M+1): m/z=:178.1.

Step B. 2-cyclopropyl-5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-2,4,5,6,7,8-hexahydropyrazolo[4,3-c]azepine: To a solution of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (150 mg, 1.0 equiv), N,N-diethylpropan-2-amine (41.85 mg, 1.5 equiv) and 4A molecular sieve (30 mg, 1.0 equiv) in DMF (0.3 mL) was added 2-cyclopropyl-5,6,7,8-tetrahydro-4H-pyrazolo[4,3-c]azepine (60 mg, 1.6 equiv). The reaction was stirred at 40° C. for 12 hours under N2 atmosphere. The mixture was diluted with water (40 mL) and extracted with ethyl acetate (2×30 mL). The combined organic layers were washed with brine (40 mL), dried over sodium sulfate, concentrated, and purified by reversed phase column (C 18, A: water[(0.1% FA)-ACN];B:ACN, B %: 45%-65%, 25 min) to afford the title compound (140 mg, 93% yield) as yellow oil; LCMS (ESI, M+1): m/z=700.2.

Step C. 4-(4-(2-cyclopropyl-7,8-dihydropyrazolo[4,3-c]azepin-5(2H,4H,6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol: To a solution of 2-cyclopropyl-5-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-2,4,5,6,7,8-hexahydropyrazolo[4,3-c]azepine (100 mg, 1.0 equiv) in MeOH (0.7 mL) was added HCl-MeOH (4 M, 350 μL 10.0 equiv). The reaction was stirred at 25° C. for 0.5 hours. The mixture was concentrated under reduced pressure and purified with prep-HPLC (column: Phenomenex luna C18 150×25 mm×10 um; A: water [(FA)-ACN];B %: 18%-48%, over 2 min) to afford the title compound (140 mg, 93% yield, FA salt) as light yellow solid. 1H NMR (400 MHz, METHANOL-d4) δ=7.55 (s, 1H), 7.53-7.48 (m, 1H), 7.14 (t, J=9.2 Hz, 1H), 6.96 (s, 2H), 5.44-5.26 (m, 1H), 4.65 (s, 2H), 4.31-4.16 (m, 1H), 4.15-3.96 (m, 4H), 3.66 (br d, J=18.0 Hz, 1H), 3.55-3.48 (m, 2H), 3.43-3.34 (m, 5H), 3.20 (br s, 3H), 2.95-2.81 (m, 2H), 2.81-2.64 (m, 1H), 2.43-2.22 (m, 2H), 2.21-2.13 (m, 1H), 2.12-2.00 (m, 3H), 1.99-1.81 (m, 2H), 1.10 (br d, J=2.0 Hz, 3H), 1.01 (br s, 4H); LCMS (ESI, M+1): m/z=656.3.

### Example 240

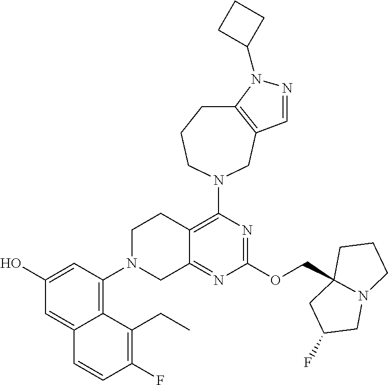 [SMILES: CCc1c(F)ccc2cc(O)cc(N3CCc4c(nc(OC[C@@]56CCCN5C[C@H](F)C6)nc4N4CCCc5c(cnn5C5CCC5)C4)C3)c12]

### 4-(4-(1-cyclobutyl-7,8-dihydropyrazolo[4,3-c]azepin-5(1H,4H,6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

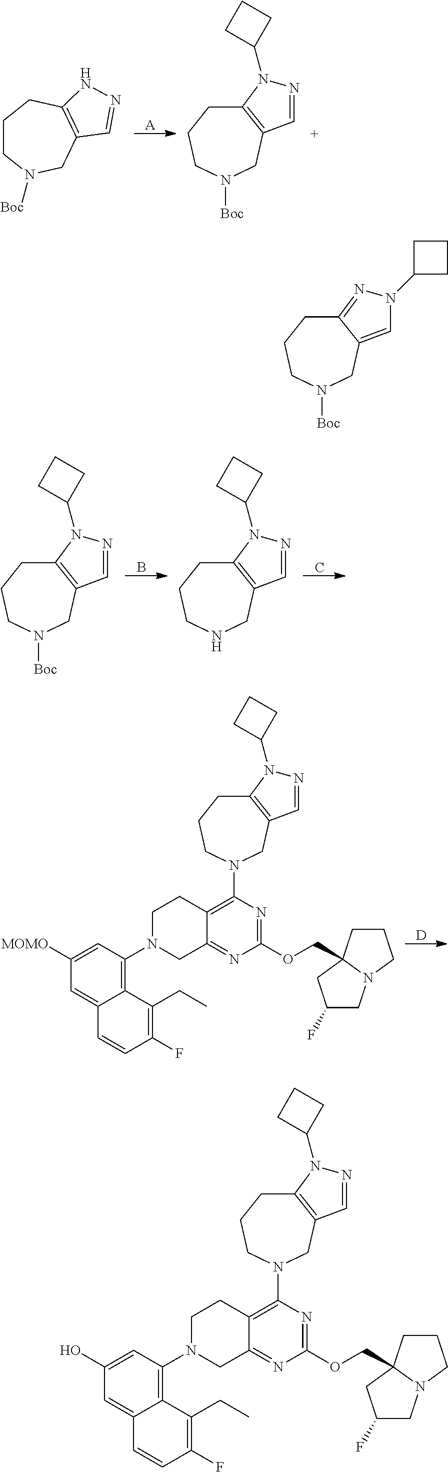

Step A. tert-butyl 2-cyclobutyl-4,6,7,8-tetrahydropyrazolo[4,3-c]azepine-5(2H)-carboxylate: To a mixture of tert-butyl 4,6,7,8-tetrahydro-1H-pyrazolo[4,3-c]azepine-5-carboxylate (850 mg, 1.0 equiv) and Cs2CO₃ (2.33 g, 2.0 equiv) in DMF (10 mL) was added iodocyclobutane (1.30 g, 2.0 equiv). The reaction was stirred at 100° C. for 3 hours under N2 atmosphere. The mixture was diluted with water (30 mL) and extracted with ethyl acetate (2×30 mL). The combined organic layers were washed with brine (30 mL), dried over sodium sulfate, concentrated, and purified with prep-HPLC (column: YMC Triart C18 150×25 mm×5 um;A: water[(FA)-ACN];B: ACN, B %: 42%-72%, over 10 min) and SFC separation (column: DAICEL CHIRALPAK IC(250 mm×30 mm,5 um);A,: [0.1% NH₃H₂O MeOH]; B, ACN, B %: 25%-25%, over 4.0 min) to afford two regioisomers,

tert-butyl 1-cyclobutyl-4,6,7,8-tetrahydropyrazolo[4,3-c]azepine-5(1H)-carboxylate (250 mg, 24% yield) as a colorless oil, 1H NMR (400 MHz, CHLOROFORM-d) δ=7.39-7.28 (m, 1H), 4.66 (quin, J=8.4 Hz, 1H), 4.43-4.26 (m, 2H), 3.71-3.56 (m, 2H), 2.81-2.76 (m, 2H), 2.67 (br t, J=9.2 Hz, 2H), 2.42-2.33 (m, 2H), 1.93-1.81 (m, 4H), 1.46-1.37 (m, 9H)

tert-butyl 2-cyclobutyl-4,6,7,8-tetrahydropyrazolo[4,3-c]azepine-5(2H)-carboxylate (250 mg, 24% yield) as a colorless oil, 1H NMR (400 MHz, CHLOROFORM-d) δ=7.48-7.31 (m, 1H), 4.70 (br d, J=8.4 Hz, 1H), 4.49-4.25 (m, 2H), 3.72 (br s, 2H), 3.03-2.81 (m, 2H), 2.69-2.35 (m, 4H), 1.88 (br s, 4H), 1.59-1.40 (m, 9H).

Step B. 1-cyclobutyl-1,4,5,6,7,8-hexahydropyrazolo[4,3-c]azepine: To a solution of tert-butyl 1-cyclobutyl-4,6,7,8-tetrahydropyrazolo[4,3-c]azepine-5(1H)-carboxylate (100 mg,1.0 equiv) in DCM (1 mL) was added TFA (1.54 g, 39 equiv). The reaction was stirred at 0-25° C. for 10 minutes under N2 atmosphere. The mixture was diluted with saturated ammonium chloride solution (20 mL) and extracted with ethyl acetate (3×30 mL). The combined organic layers were washed with brine (20 mL), dried over sodium sulfate, and concentrated to afford the title compound (60.0 mg, 91% yield) as yellow solid. LCMS (ESI, M+1): m/z=192.1.

Step C. 1-cyclobutyl-5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-1,4,5,6,7,8-hexahydropyrazolo[4,3-c]azepine: To a mixture of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (160 mg, 1.0 equiv), N,N-diethylpropan-2-amine (44.6 mg, 1.5 equiv) and 4A molecular sieve (30 mg,1.0 equiv) in DMF (0.3 mL) was added 2-cyclobutyl-2,4,5,6,7,8-hexahydropyrazolo[4,3-c]azepine (60 mg, 1.4 equiv). The reaction was stirred at 40° C. for 12 hours under N2 atmosphere. The mixture was diluted with water (40 mL) and extracted with ethyl acetate (2×30 mL). The combined organic layers were washed with brine (40 mL), dried over sodium sulfate, concentrated, and purified by reversed phase column (C 18, mobile phase: [water(0.1% FA)-ACN]; B %: 45%-65%, 25 min) to afford the title compound (140 mg, 85.1% yield) as yellow oil; LCMS (ESI, M+1): m/z=:714.2.

Step D. 4-(4-(1-cyclobutyl-7,8-dihydropyrazolo[4,3-c]azepin-5(1H,4H,6H)-yl)-2-(((2R,7aS)-2-fluorohexahydro-1H-pyrrolizin-7a-yl)methoxy)-5,6-dihydropyrido[3,4-d] pyrimidin-7(8H)-yl)-5-ethyl-6-fluoronaphthalen-2-ol: To a solution of 1-cyclobutyl-5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,4,5,6,7,8-hexahydropyrazolo[4,3-c]azepine (100 mg, 1.0 equiv) in MeOH (0.8 mL) was added HCl-MeOH (4 M, 350 μL10.0 equiv). The reaction was stirred at 25° C. for 0.5 hours under N2 atmosphere. The mixture was concentrated and purified with prep-HPLC (column: Phenomenex luna C18 150 x 25 mm×10 um; A: water[(FA)-ACN];B: ACN, B %: 18%-48%, over 2 min) to afford the title compound (33.1 mg, 35.3% yield, FA salt) as light yellow solid; 1H NMR (400 MHz, METHANOL-d4) δ=7.56-7.48 (m, 1H), 7.44 (d, J=3.2 Hz, 1H), 7.20-7.07 (m, 1H), 6.96 (s, 2H), 5.55-5.26 (m, 1H), 4.84-4.58 (m, 3H), 4.36-4.17 (m, 2H), 4.13-3.94 (m, 3H), 3.62 (br d, J=17.6 Hz, 1H), 3.59-3.44 (m, 4H), 3.40-3.33 (m, 2H), 3.28-3.17 (m, 2H), 3.16-3.03 (m, 1H), 3.02-2.90 (m, 1H), 2.89-2.78 (m, 1H), 2.72 (br d, J=14.6 Hz, 1H), 2.65-2.53 (m, 2H), 2.43-2.27 (m, 4H), 2.26-2.08 (m, 4H), 2.07-1.93 (m, 2H), 1.92-1.79 (m, 2H), 1.17-1.06 (m, 3H) LCMS (ESI, M+1): m/z=670.4.

### Example 241

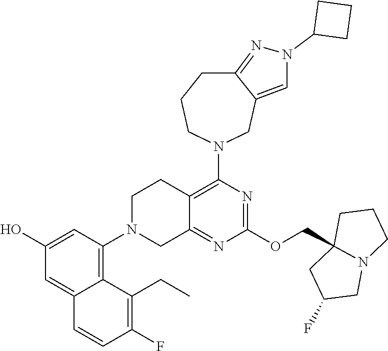 [SMILES: CCc1c(F)ccc2cc(O)cc(N3CCc4c(nc(OC[C@@]56CCCN5C[C@H](F)C6)nc4N4CCCc5nn(C6CCC6)cc5C4)C3)c12]

### 4-(4-(2-cyclobutyl-7,8-dihydropyrazolo[4,3-c]azepin-5(2H,4H,6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

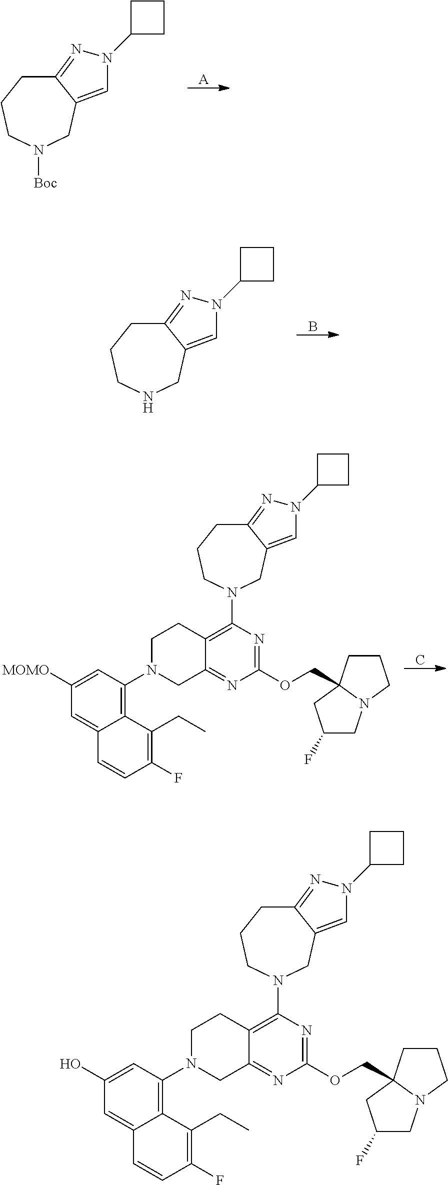

Step A. 2-cyclobutyl-2,4,5,6,7,8-hexahydropyrazolo[4,3-c]azepine: To a solution of tert-butyl 2-cyclobutyl-4,6,7,8-tetrahydropyrazolo[4,3-c]azepine-5(2H)-carboxylate (100 mg,1.0 equiv) in DCM (1 mL) was added TFA (1.54 g, 39 equiv). The reaction was stirred at 0-25° C. for 10 minutes under N2 atmosphere. The mixture was diluted with saturated ammonium chloride solution (20 mL) and extracted with ethyl acetate (3×30 mL). The combined organic extracts were washed with brine (20 mL), dried over sodium sulfate, and concentrated to afford the title compound (60.0 mg, 91% yield) as yellow solid; LCMS (ESI, M+1): m/z=:192.1.

Step B. 2-cyclobutyl-5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-2,4,5,6,7,8-hexahydropyrazolo[4,3-c]azepine: To a mixture of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (160 mg, 1.0 equiv), N,N-diethylpropan-2-amine (44.6 mg, 1.5 equiv) and 4A molecular sieve (30 mg,1.0 equiv) in DMF (0.3 mL) was added 2-cyclobutyl-5,6,7,8-tetrahydro-4H-pyrazolo[4,3-c]azepine (60 mg, 1.4 equiv). The reaction was stirred at 40° C. for 12 hours under N2 atmosphere. The mixture was diluted with water (40 mL) and extracted with ethyl acetate (2×30 mL). The combined organic layers were washed with brine (40 mL), dried over sodium sulfate, concentrated, and purified by reversed phase column (C 18, mobile phase: [water (0.1% FA)-ACN]; B %: 45%-65%, 25 min) to afford the title compound (140 mg, 85% yield) as yellow oil; LCMS (ESI, M+1): m/z=714.2.

Step C. 4-(4-(2-cyclobutyl-7,8-dihydropyrazolo[4,3-c]azepin-5(2H,4H,6H)-yl)-2-(((2R,7aS)-2-fluorohexahydro-1H-pyrrolizin-7a-yl)methoxy)-5,6-dihydropyrido[3,4-d] pyrimidin-7(8H)-yl)-5-ethyl-6-fluoronaphthalen-2-ol: To a solution of 2-cyclobutyl-5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-2,4,5,6,7,8-hexahydropyrazolo[4,3-c]azepine (100 mg, 1.0 equiv) in MeOH (0.8 mL) was added HCl-MeOH (4 M, 350 μL10 equiv). The reaction was stirred at 25° C. for 0.5 hours under N2 atmosphere. The mixture was concentrated and purified with prep-HPLC(Phenomenex luna C18 150×25 mm×10 um; A: water[(FA)-ACN];B: ACN, B %: 18%-48%, over 2 min) to afford the title compound (33.1 mg, 35% yield, FA salt) as light yellow solid; 1H NMR (400 MHz, METHANOL-d4) δ=7.67-7.56 (m, 1H), 7.50 (dd, J=5.6, 8.8 Hz, 1H), 7.20-7.08 (m, 1H), 6.95 (s, 2H), 5.42-5.19 (m, 1H), 4.80-4.62 (m, 3H), 4.28-4.14 (m, 1H), 4.00 (s, 4H), 3.70-3.61 (m, 1H), 3.57-3.40 (m, 2H), 3.39-3.33 (m, 3H), 3.26 (br s, 1H), 3.24-3.11 (m, 2H), 3.10-3.01 (m, 1H), 2.99-2.84 (m, 2H), 2.81-2.65 (m, 1H), 2.54-2.36 (m, 4H), 2.35-2.17 (m, 2H), 2.17-1.97 (m, 4H), 1.96-1.78 (m, 4H), 1.15-1.01 (m, 3H); LCMS (ESI, M+1): m/z=670.4.

### Example 242

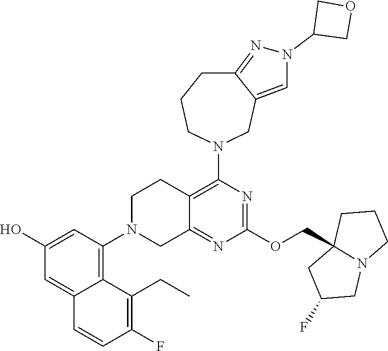 [SMILES: CCc1c(F)ccc2cc(O)cc(N3CCc4c(nc(OC[C@@]56CCCN5C[C@H](F)C6)nc4N4CCCc5nn(C6COC6)cc5C4)C3)c12]

### 5-ethyl-6-fluoro-4-(2-(((2R,7aS)-2-fluorohexahydro-1H-pyrrolizin-7a-yl)methoxy)-4-(2-(oxetan-3-yl)-7,8-dihydropyrazolo[4,3-c]azepin-5(2H,4H,6H)-yl)-5,6-dihydropyrido[3,4-d]pyrimidin-7(8H)-yl)naphthalen-2-ol

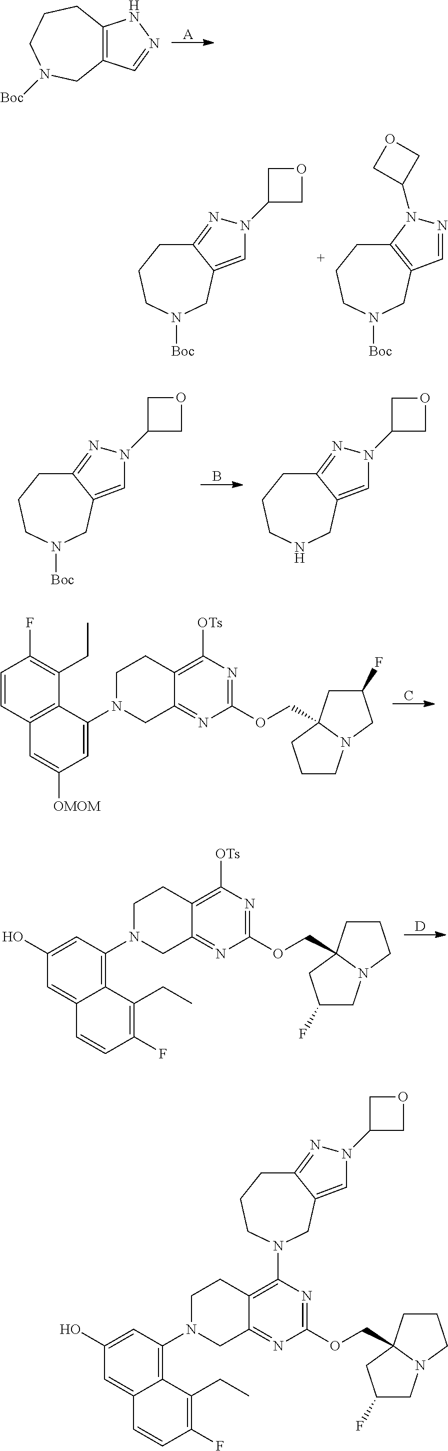

Step A. tert-butyl 2-(oxetan-3-yl)-4,6,7,8-tetrahydropyrazolo[4,3-c]azepine-5(2H)-carboxylate: A mixture of tert-butyl 4,6,7,8-tetrahydro-1H-pyrazolo[4,3-c]azepine-5-carboxylate (600 mg, 1.0 equiv), 3-iodooxetane (559 mg, 1.2 equiv), and Cs2CO₃ (822 mg,2.0 equiv) in DMF (6 mL) was degassed and purged with N2 3 times. The reaction was stirred at 100° C. for 1 hour under N2 atmosphere. The mixture was diluted with water (40 mL) and extracted with ethyl acetate (2×30 mL). The combined organic layers were washed with brine (40 mL), dried over sodium sulfate, concentrated, and purified with prep-HPLC (column: YMC Triart C18 150*25 mm*5 um;mobile phase: [water(FA)-ACN];B %: 28%-58%,10 min) and SFC separation (column: DAICEL CHIRALPAK IC(250 mm*30 mm,5 um);mobile phase:

[0.1% NH₃H₂OMeOH];B %: 35%-35%,3.5 min) to afford two regioisomers,

tert-butyl 2-(oxetan-3-yl)-4,6,7,8-tetrahydropyrazolo[4,3-c]azepine-5(2H)-carboxylate (220 mg, 30% yield) as a colorless oil; 1H NMR (400 MHz, CHLOROFORM-d) δ=7.48-7.32 (m, 1H), 5.44-5.22 (m, 1H), 5.15-4.90 (m, 4H), 4.48-4.19 (m, 2H), 3.66 (br s, 2H), 3.12-2.80 (m, 2H), 1.90-1.75 (m, 2H), 1.42 (br s, 9H).

tert-butyl 1-(oxetan-3-yl)-4,6,7,8-tetrahydropyrazolo[4,3-c]azepine-5(1H)-carboxylate (120 mg, 16% yield) as a colorless oil; 1H NMR (400 MHz, CHLOROFORM-d) δ=7.47-7.35 (m, 1H), 5.40-5.38 (m, 1H), 5.24-5.11 (m, 2H), 5.06-4.92 (m, 2H), 4.45-4.25 (m, 2H), 3.74-3.58 (m, 2H), 2.82-2.70 (m, 2H), 1.93-1.84 (m, 2H), 1.41 (brs, 9H)

Step B. 2-(oxetan-3-yl)-2,4,5,6,7,8-hexahydropyrazolo[4,3-c]azepine: To a solution of tert-butyl 2-(oxetan-3-yl)-4,6,7,8-tetrahydropyrazolo[4,3-c]azepine-5(2H)-carboxylate (100 mg, 1.0 equiv) in DCM (1 mL) was added TFA (1.54 g, 39.6 equiv). The reaction was degassed and purged with N2 3 times, and then stirred at 0-25° C. for 10 minutes under N2 atmosphere. The mixture was concentrated to afford the title compound (60.0 mg, 91% yield) as a yellow solid. LCMS (ESI, M+1): m/z=194.0.

Step C. 7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate: To a solution of 7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl4-methylbenzenesulfonate (140 mg, 1.0 equiv) in DCM (2 mL) was added TFA (770 mg, 33.5 equiv). The reaction was stirred at 0-25° C. for 1.5 hours. The mixture was quenched with saturated NaHCO₃ (20 mL). The mixture was extracted with ethyl acetate (3×30 mL). The combined organic layers were washed with brine (20 mL), dried over sodium sulfate, concentrated, and purified by reversed phase column (C 18, mobile phase: [water(0.1% FA)-ACN]; B %: 45%-65%, 25 min) to afford the title compound (90 mg, 68% yield) as a yellow solid; LCMS (ESI, M+1): m/z=:651.2.

Step D. 5-ethyl-6-fluoro-4-(2-(((2R,7aS)-2-fluorohexahydro-1H-pyrrolizin-7a-yl)methoxy)-4-(2-(oxetan-3-yl)-7,8-dihydropyrazolo[4,3-c]azepin-5(2H,4H,6H)-yl)-5,6-dihydropyrido[3,4-d]pyrimidin-7(8H)-yl)naphthalen-2-ol: To a solution of 7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (90 mg, 1.0 equiv), N,N-diethylpropan-2-amine (26.8 mg, 1.5 equiv), and 4A molecular sieve (20 mg, 1.0 equiv) in DMF (0.9 mL) was added 2-(oxetan-3-yl)-5,6,7,8-tetrahydro-4H-pyrazolo[4,3-c]azepine (90 mg, 3.4 equiv). The reaction was degassed and purged with N2 3 times, and then stirred at 40° C. for 12 hours under N2 atmosphere. The mixture was diluted with water (10 mL) and extracted with ethyl acetate (2×30 mL). The combined organic layers were washed with brine (10 mL), dried over sodium sulfate, concentrated, and purified by reversed phase column (column: Waters Xbridge 150*25 mm*Sum;mobile phase: [water(NH₄HCO₃)-ACN];B %: 46%-76%,8 min) to afford the title compound (13.2 mg, 7% yield) as a pink solid. 1H NMR (400 MHz, METHANOL-d4) δ=7.56 (s, 1H), 7.51 (dd, J=5.6, 9.2 Hz, 1H), 7.14 (t, J=9.2 Hz, 1H), 6.96 (s, 2H), 5.68-5.55 (m, 1H), 5.40-5.22 (m, 1H), 5.08-5.05 (m, 2H), 5.01-4.96 (m, 4H), 4.84-4.76 (m, 1H), 4.70-4.62 (m, 1H), 4.60 (s, 1H), 4.21-4.06 (m, 3H), 4.01 (br d, J=17.9 Hz, 1H), 3.70-3.56 (m, 1H), 3.56-3.46 (m, 1H), 3.36 (br d, J=7.5 Hz, 3H), 3.25 (br s, 1H), 3.24-3.11 (m, 2H), 3.10-3.00 (m, 1H), 2.96-2.82 (m, 2H), 2.78-2.66 (m, 1H), 2.35-2.10 (m, 4H), 2.07-1.86 (m, 4H), 1.10 (dt, J=1.5, 7.2 Hz, 3H); LCMS (ESI, M+1): m/z=:672.4.

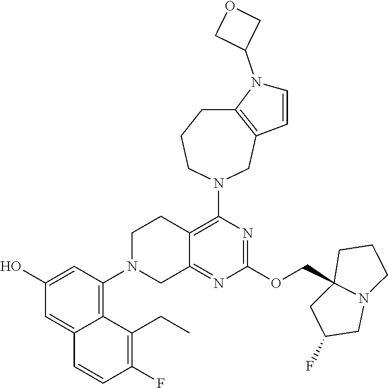 [SMILES: CCc1c(F)ccc2cc(O)cc(N3CCc4c(nc(OC[C@@]56CCCN5C[C@H](F)C6)nc4N4CCCc5c(ccn5C5COC5)C4)C3)c12]

### Example 243

### 5-ethyl-6-fluoro-4-(2-(((2R,7aS)-2-fluorohexahydro-1H-pyrrolizin-7a-yl)methoxy)-4-(1-(oxetan-3-yl)-7,8-dihydropyrazolo[4,3-c]azepin-5(1H,4H,6H)-yl)-5,6-dihydropyrido[3,4-d]pyrimidin-7(8H)-yl)naphthalen-2-ol

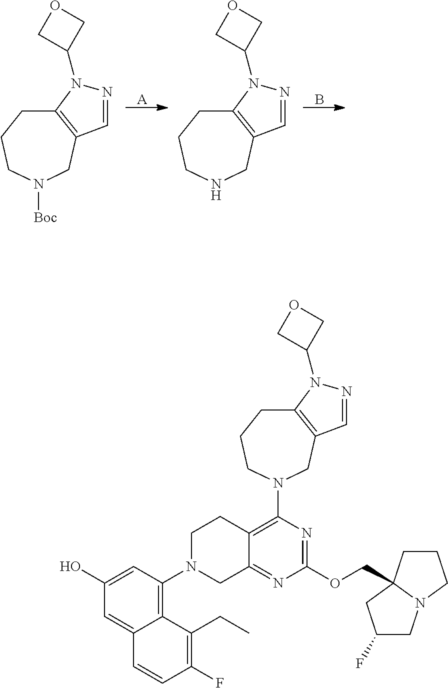

Step A. 1-(oxetan-3-yl)decahydropyrazolo[4,3-c]azepine: To a solution of tert-butyl 1-(oxetan-3-yl)-4,6,7,8-tetrahydropyrazolo[4,3-c]azepine-5(1H)-carboxylate (100 mg, 1.0 equiv) in DCM (1 mL) was added TFA (1.54 g, 39.6 equiv). The reaction was stirred at 0-25° C. for 10 minutes under N2 atmosphere. The mixture was concentrated to afford the title compound (60.0 mg, 91% yield) as a yellow solid. LCMS (ESI, M+1): m/z=194.2.

Step B. 5-ethyl-6-fluoro-4-(2-(((2R,7aS)-2-fluorohexahydro-1H-pyrrolizin-7a-yl)methoxy)-4-(1-(oxetan-3-yl)-7,8-dihydropyrazolo[4,3-c]azepin-5(1H,4H,6H)-yl)-5,6-dihydropyrido[3,4-d]pyrimidin-7(8H)-yl)naphthalen-2-ol: To a mixture of 7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (180 mg, 1.0 equiv), N,N-diethylpropan-2-amine (35.7 mg, 1.0 equiv) and 4A molecular sieve (20 mg, 1.0 equiv) in DMF (0.3 mL) was added 1-(oxetan-3-yl)-1,4,5,6,7,8-hexahydropyrazolo[4,3-c]azepine (30 mg, 0.56 equiv). The reaction was stirred at 40° C. for 12 hours under N2 atmosphere. The mixture was diluted with water (40 mL) and extracted with ethyl acetate (2×30 mL). The combined organic layers were washed with brine (40 mL), dried over sodium sulfate, and purified with prep-HPLC [waters Xbridge 150×25 mm×5 μm; A: water (10 mM NH₄HCO₃), B: ACN, B %: 46%-76% over 8 min] to afford the title compound (13.2 mg, 7% yield) as a pink solid. 1H NMR (400 MHz, METHANOL-d4) δ=7.65 (d, J=3.2 Hz, 1H), 7.58-7.44 (m, 1H), 7.22-7.07 (m, 1H), 7.03-6.86 (m, 2H), 5.42 (quin, J=6.8 Hz, 1H), 5.36-5.17 (m, 1H), 5.00 (br dd, J=4.8, 7.2 Hz, 4H), 4.97-4.93 (m, 2H), 4.82-4.66 (m, 3H), 4.26-4.15 (m, 1H), 4.08-3.96 (m, 3H), 3.73-3.64 (m, 1H), 3.51-3.46 (m, 1H), 3.23 (br s, 2H), 3.20-3.09 (m, 3H), 3.06-2.93 (m, 3H), 2.78-2.68 (m, 1H), 2.23-2.03 (m, 4H), 2.02-1.80 (m, 4H), 1.16-1.01 (m, 3H); LCMS (ESI, M+1): m/z=672.4.

### Example 244

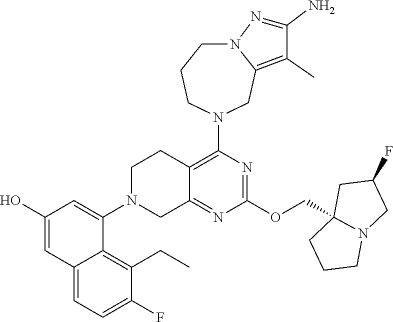 [SMILES: CCc1c(F)ccc2cc(O)cc(N3CCc4c(nc(OC[C@@]56CCCN5C[C@H](F)C6)nc4N4CCCn5nc(N)c(C)c5C4)C3)c12]

### 4-(4-(2-amino-3-methyl-7,8-dihydro-4H-pyrazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

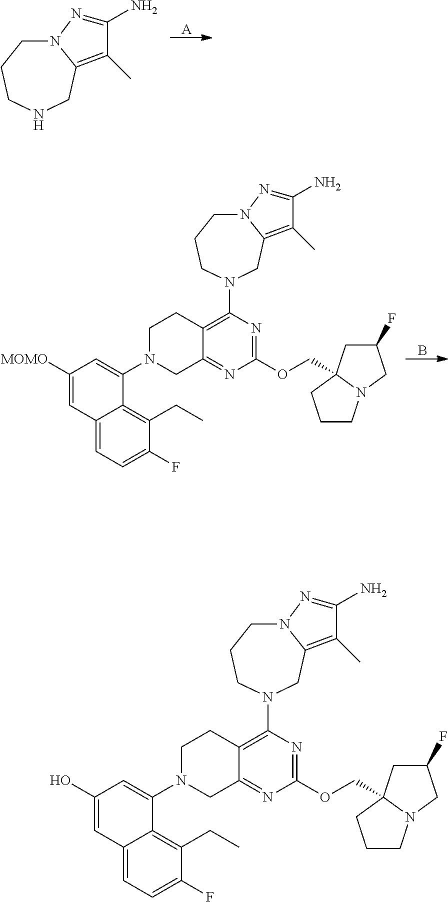

Step A. 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-3-methyl-5,6,7,8-tetrahydro-4H-pyrazolo[1,5-a][1,4]diazepin-2-amine: To a solution of 3-methyl-5,6,7,8-tetrahydro-4H-pyrazolo[1,5-a][1,4]diazepin-2-amine (105 mg, 2.0 equiv, HCl) in DMF (5 mL) was added N,N-diethylpropan-2-amine (167 mg, 5.0 equiv) and 7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (180 mg, 1.0 equiv). The mixture was stirred at 40° C. for 14 hours. The mixture was diluted with water (20 mL) and extracted with ethyl acetate (50 mL). The organic layer was dried over sodium sulfate, concentrated, and purified by reversed phase HPLC (C18, 0.1% FA condition) to afford the title compound (150 mg, 65% yield) as yellow solid; LCMS (ESI, M+1): m/z=689.3.

Step B. 4-(4-(2-amino-3-methyl-7,8-dihydro-4H-pyrazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol: To a solution of 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-3-methyl-5,6,7,8-tetrahydro-4H-pyrazolo[1,5-a][1,4]diazepin-2-amine (19 mg, 1.0 equiv) in MeOH (1 mL) was added HCl/MeOH (4 M, 2 mL, 290 equiv) at 0° C. The mixture was stirred at 0° C. for 0.5 hours. The mixture was concentrated and purified with prep-HPLC (Phenomenex Luna C18 150×25 mm×10 μm; mobile phase: A: water (FA), B: ACN; B %: 15% to 45% over 10 min) to afford the title compound (5.01 mg, 26% yield, FA salt) as yellow solid; 1H NMR (400 MHz, METHANOL-d4) δ=7.52 (dd, J=6.0, 9.2 Hz, 1H), 7.15 (t, J=9.2 Hz, 1H), 7.02-6.93 (m, 2H), 5.53-5.30 (m, 1H), 4.69-4.56 (m, 2H), 4.32-4.13 (m, 4H), 4.12-3.98 (m, 2H), 3.95-3.83 (m, 1H), 3.71-3.61 (m, 1H), 3.59-3.47 (m, 3H), 3.46-3.37 (m, 2H), 3.25-3.11 (m, 3H), 2.78-2.65 (m, 1H), 2.50-2.31 (m, 2H), 2.29-1.97 (m, 7H), 1.97-1.90 (m, 3H), 1.10 (t, J=6.8 Hz, 3H). LCMS (ESI, M+1): m/z=645.5.

### Example 245

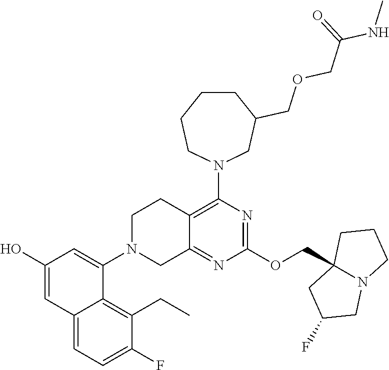 [SMILES: CCc1c(F)ccc2cc(O)cc(N3CCc4c(nc(OC[C@@]56CCCN5C[C@H](F)C6)nc4N4CCCCC(COCC(=O)NC)C4)C3)c12]

### 2-((1-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)azepan-3-yl)methoxy)-N-methylacetamide

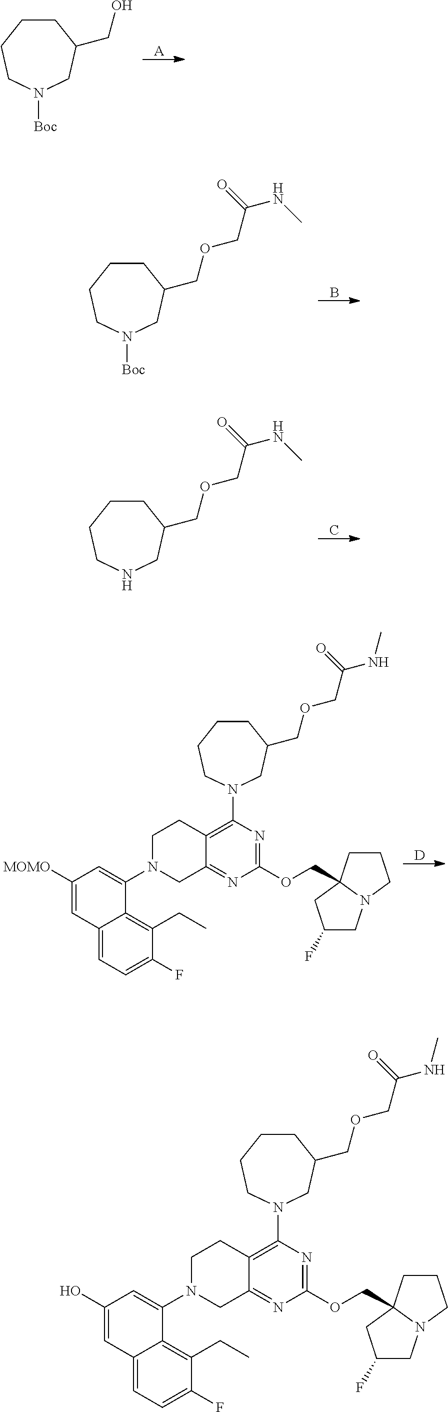

Step A: tert-butyl 3-((2-(methylamino)-2-oxoethoxy)methyl)azepane-1-carboxylate: To a solution of tert-butyl 3-(hydroxymethyl)azepane-1-carboxylate (300 mg, 1.0 equiv) in THF (5.0 mL) was added NaH (157 mg, 60% purity, 3.0 equiv) at 0° C. slowly. The mixture was stirred at 0° C. for 1 hour. Then 2-bromo-N-methyl-acetamide (238 mg, 1.2 equiv) was added dropwise at 0° C. The resulting mixture was stirred at 25° C. for 5 hours. The reaction was quenched with water (20 mL) at 0° C. and extracted with EtOAc (2×30 mL). The combined organic layers were dried over anhydrous sodium sulfate and concentrated to give a residue. The residue was purified with column chromatography [SiO₂, Petroleum ether/Ethyl acetate=5/1 to 1/1] to afford the title compound (200 mg, 51% yield) as yellow oil.

Step B: 2-(azepan-3-ylmethoxy)-N-methylacetamide: To a solution of tert-butyl 3-((2-(methylamino)-2-oxoethoxy)methyl)azepane-1-carboxylate (100 mg, 1 equiv) in EtOAc (1.0 mL) was added HCl/EtOAc (1.0 mL, 4.0 M, 6.0 equiv). The mixture was stirred at 25° C. for 1 hour. The reaction was concentrated to afford the title compound (60 mg, 90% yield).

Step C: 2-((1-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)azepan-3-yl)methoxy)-N-methylacetamide: To a solution of 2-(azepan-3-ylmethoxy)-N-methylacetamide (57 mg, 4.0 equiv) in DMF (2.0 mL) were added N,N-diethylpropan-2-amine (93.0 mg, 10 equiv) and 7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (50.0 mg, 1.0 equiv). The mixture was stirred at 40° C. for 12 hours. The reaction was diluted with water (30 mL) and extracted with ethyl acetate (40 mL). The organic phase was dried over Na₂SO₄, concentrated, and purified with prep-TLC [DCM/MeOH=10/1] to afford the title compound (40 mg, 60% yield) as yellow solid. LCMS (ESI, M+1): m/z=723.3

Step D: 2-((1-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)azepan-3-yl)methoxy)-N-methylacetamide: To a solution of 2-((1-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)azepan-3-yl)methoxy)-N-methylacetamide (40.0 mg, 1.0 equiv) in MeCN (1.0 mL) was added HCl/dioxane (1.0 mL, 4.0 M, 10 equiv). The mixture was stirred at 0° C. for 1 hour. The reaction was concentrated and purified with prep-HPLC [column: Phenomenex C18 75×30 mm×3 μm; mobile phase: [water (FA)-ACN]; B %: 18%-48%, 7 minutes] and prep-HPLC [column: Waters Xbridge 150×25 mm×5 μm; mobile phase: [water (ammonia hydroxide v/v)-ACN]; B %: 48%-78%, 9 minutes] to afford the title compound (12.4 mg, 32% yield) as yellow solid. 1H NMR (400 MHz, DMSO-d6) δ=9.70 (d, J=7.0 Hz, 1H), 7.58 (br d, J=6.2 Hz, 2H), 7.23 (t, J=9.2 Hz, 1H), 7.02-6.97 (m, 2H), 5.46-5.22 (m, 1H), 4.03 (br s, 3H), 3.84 (br d, J=6.6 Hz, 4H), 3.58 (br d, J=17.6 Hz, 2H), 3.38 (br s, 2H), 3.21-3.06 (m, 5H), 2.91 (br s, 1H), 2.75-2.65 (m, 1H), 2.60 (dd, J=2.0, 4.6 Hz, 3H), 2.39-1.60 (m, 13H), 1.54-1.20 (m, 3H), 1.07-1.01 (m, 3H); LCMS (ESI, M+1): m/z=679.2.

### Example 246

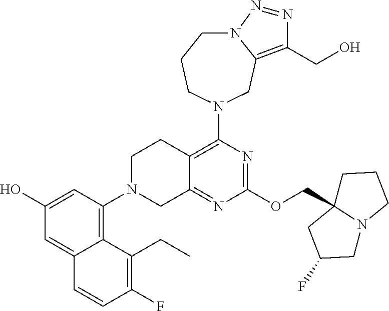 [SMILES: CCc1c(F)ccc2cc(O)cc(N3CCc4c(nc(OC[C@@]56CCCN5C[C@H](F)C6)nc4N4CCCn5nnc(CO)c5C4)C3)c12]

### 5-ethyl-6-fluoro-4-(2-(((2R,7aS)-2-fluorohexahydro-1H-pyrrolizin-7a-yl)methoxy)-4-(3-(hydroxymethyl)-7,8-dihydro-4H-[1,2,3]triazolo[1,5-a][1,4]diazepin-5(6H)-yl)-5,6-dihydropyrido[3,4-d]pyrimidin-7(8H)-yl)naphthalen-2-ol

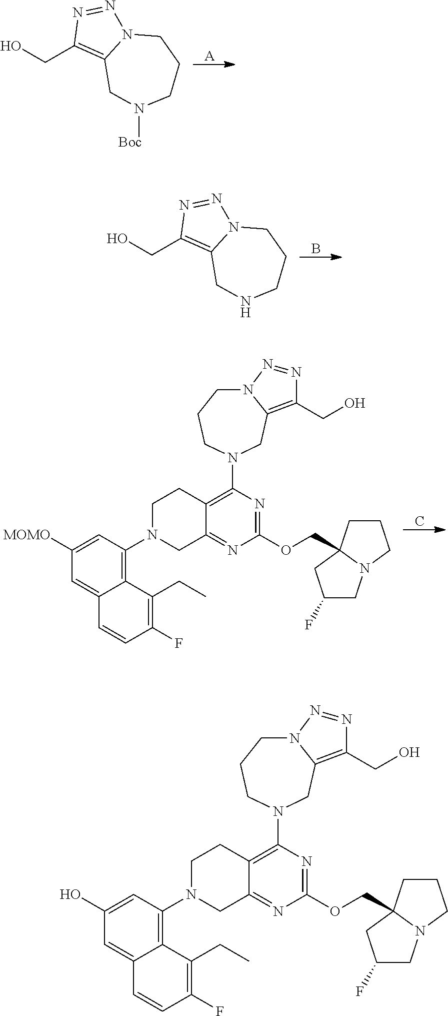

Step A. (5,6,7,8-tetrahydro-4H-[1,2,3]triazolo[1,5-a][1,4]diazepin-3-yl)methanol: To a solution of tert-butyl 3-(hydroxymethyl)-7,8-dihydro-4H-[1,2,3]triazolo[1,5-a][1,4]diazepine-5(6H)-carboxylate (220 mg, 1.0 equiv) in MeCN (1 mL) was added HCl•dioxane (2 mL, 9.8 equiv). The reaction was stirred at 25° C. for 0.5 hours. The mixture was concentrated to afford the title compound (240 mg, crude, HCl) as yellow solid and was used into next step without further purification.

Step B. (5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-[1,2,3]triazolo[1,5-a][1,4]diazepin-3-yl)methanol: To a mixture of 7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (80.0 mg, 1.0 equiv) and (5,6,7,8-tetrahydro-4H-[1,2,3]triazolo[1,5-a][1,4]diazepin-3-yl)methanol (38.7 mg, 2.0 equiv) in DMF (0.5 mL) were added N,N-diethylpropan-2-amine (149 mg, 10 equiv) and 4 A molecular sieve (10 mg). The reaction was stirred at 40° C. for 12 hours. The mixture was filtered and purified by reversed phase flash chromatography [C18, water (FA, 0.1%)/acetonitrile] to afford the title compound (70.0 mg, 87% yield) as yellow solid. LCMS (ESI, M+1): m/z=691.4.

Step C. 5-ethyl-6-fluoro-4-(2-((2R,7aS)-2-fluorohexahydro-1H-pyrrolizin-7a-yl)methoxy)-4-(3-(hydroxymethyl)-7,8-dihydro-4H-[1,2,3]triazolo[1,5-a][1,4]diazepin-5(6H)-yl)-5,6-dihydropyrido[3,4-d]pyrimidin-7(8H)-yl)naphthalen-2-ol: A mixture of (5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-[1,2,3]triazolo[1,5-a][1,4]diazepin-3-yl)methanol (50.0 mg, 1.0 equiv) in HCl•dioxane (0.5 mL, 4M, 30 equiv) was stirred at 25° C. for 0.5 hours. The mixture was concentrated and basified by NaHCO₃ to pH=7 and extracted with ethyl acetate (3×10 mL). The combined organic layers were dried over by Na₂SO₄, filtered and concentrated under vacuum to give a residue. The residue was purified by prep-HPLC (column: Phenomenex luna C18 150×25 mm×10 μm; mobile phase: [water (FA) -ACN]; B %: 15%-45%, 10 min) to afford the title compound (30 mg, 63% yield, 0.53 FA) as off-white solid. 1H NMR (400 MHz, ACETIC ACID-d4) δ=7.53 (dd, J=6.0, 9.2 Hz, 1H), 7.17 (t, J=9.2 Hz, 1H), 7.11-7.01 (m, 2H), 5.69-5.45 (m, 1H), 5.27-4.99 (m, 2H), 4.94-4.78 (m, 3H), 4.78-4.59 (m, 2H), 4.51 (br dd, J=12.0, 14.0 Hz, 1H), 4.33-4.14 (m, 3H), 4.13-3.95 (m, 2H), 3.88 (br dd, J=7.6, 18.4 Hz, 1H), 3.76 (br t, J=16.0 Hz, 1H), 3.54-3.33 (m, 4H), 3.30-3.13 (m, 2H), 2.85-2.62 (m, 2H), 2.57-2.41 (m, 2H), 2.27 (br s, 5H), 1.14 (br t, J=7.2 Hz, 3H). LCMS (ESI, M+1): m/z=647.4.

### Example 247

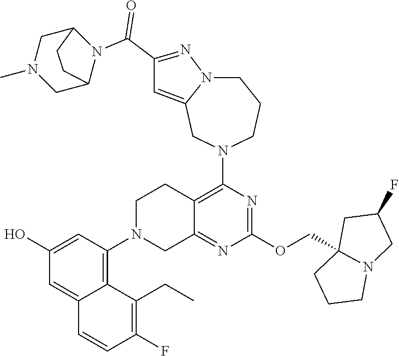 [SMILES: CCc1c(F)ccc2cc(O)cc(N3CCc4c(nc(OC[C@@]56CCCN5C[C@H](F)C6)nc4N4CCCn5nc(C(=O)N6C7CCC6CN(C)C7)cc5C4)C3)c12]

### (5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(3-methyl-3,8-diazabicyclo[3.2.1]octan-8-yl)methanone

Synthesized according to Example 233 except that HCl instead of TFA was used in the last step. The title compound was obtained as orange solid (FA salt).1H NMR (400 MHz, dimethylsulfoxide-d6+deuterium oxide-d2) δ=7.56 (dd, J=6.0, 8.8 Hz, 1H), 7.23 (t, J=9.6 Hz, 1H), 6.98 (s, 2H), 6.59 (s, 1H), 5.38-5.18 (m, 1H), 5.11 (br s, 1H), 5.00 (br dd, J=10.4, 15.6 Hz, 1H), 4.82-4.67 (m, 1H), 4.54 (br d, J=6.0 Hz, 1H), 4.47 (br d, J=5.2 Hz, 2H), 4.26-4.11 (m, 1H), 4.05-3.88 (m, 2H), 3.87-3.77 (m, 2H), 3.56 (br d, J=17.6 Hz, 1H), 3.47-3.39 (m, 1H), 3.29-3.03 (m, 7H), 2.93-2.82 (m, 1H), 2.72-2.61 (m, 3H), 2.20-2.03 (m, 8H), 2.00-1.63 (m, 9H), 1.08-0.98 (m, 3H); 19F NMR (400 MHz, dimethylsulfoxide-d6+deuterium oxide-d2) δ=−121.048, -171.962; LCMS (ESI, M+1): m/z=768.5.

### Example 248

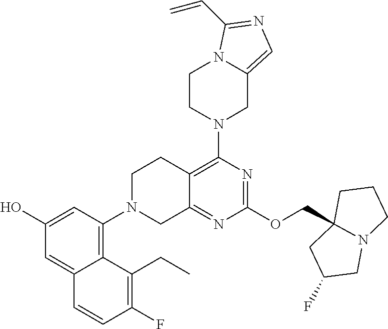 [SMILES: C=Cc1ncc2n1CCN(c1nc(OC[C@@]34CCCN3C[C@H](F)C4)nc3c1CCN(c1cc(O)cc4ccc(F)c(CC)c14)C3)C2]

### 5-ethyl-6-fluoro-4-(2-(((2R,7aS)-2-fluorohexahydro-1H-pyrrolizin-7a-yl)methoxy)-4-(3-vinyl-5,6-dihydroimidazo[1,5-a]pyrazin-7(8H)-yl)-5,6-dihydropyrido[3,4-d]pyrimidin-7(8H)-yl)naphthalen-2-ol

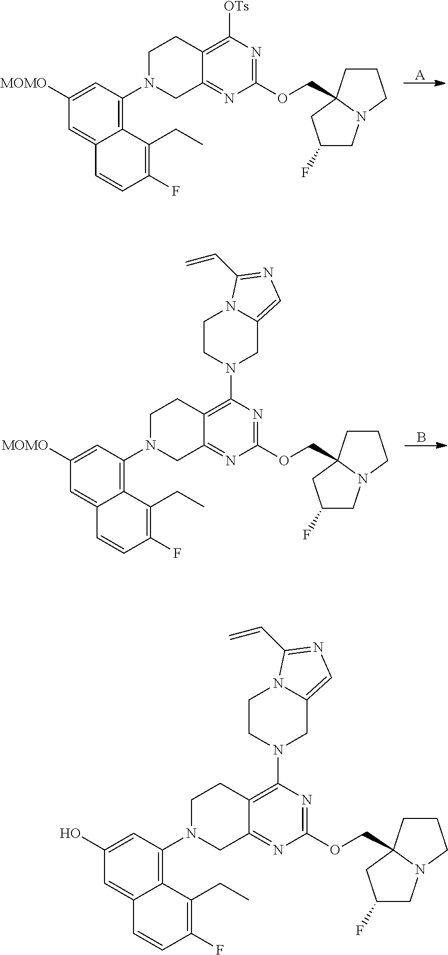

Step A. 7-(8-ethyl-7-fluoro-3-(methoxymethoxy)naphthalen-1-yl)-2-(((2R,7aS)-2-fluorohexahydro-1H-pyrrolizin-7a-yl)methoxy)-4-(3-vinyl-5,6-dihydroimidazo[1,5-a]pyrazin-7(8H)-yl)-5,6,7,8-tetrahydropyrido[3,4-d]pyrimidine: To a mixture of 7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (44.9 mg, 1.0 equiv) and 3-vinyl-5,6,7,8-tetrahydroimidazo[1,5-a]pyrazine (60.0 mg, 5.0 equiv, HCl) in DMF (1.0 mL) were added N,N-diethylpropan-2-amine (83.5 mg, 10 equiv) and 4A molecular sieve (10.0 mg). The reaction was stirred at 50° C. for 12 hours. The mixture was filtered and purified by reversed phase HPLC (0.1% FA condition) to afford the title compound (27.0 mg, 62% yield) as yellow solid; LCMS (ESI, M+1): m/z=672.4.

Step B. 5-ethyl-6-fluoro-4-(2-(((2R,7aS)-2-fluorohexahydro-1H-pyrrolizin-7a-yl)methoxy)-4-(3-vinyl-5,6-dihydroimidazo[1,5-a]pyrazin-7(8H)-yl)-5,6-dihydropyrido[3,4-d]pyrimidin-7(8H)-yl)naphthalen-2-ol: To a solution of 7-(8-ethyl-7-fluoro-3-(methoxymethoxy)naphthalen-1-yl)-2-(((2R,7aS)-2-fluorohexahydro-1H-pyrrolizin-7a-yl)methoxy)-4-(3-vinyl-5,6-dihydroimidazo[1,5-a]pyrazin-7(8H)-yl)-5,6,7,8-tetrahydropyrido[3,4-d]pyrimidine (27.0 mg, 1.0 equiv) in MeOH (0.3 mL) was added HCl-MeOH (4 M, 0.5 mL, 50 equiv). The reaction was stirred at 20° C. for 0.5 hours. The mixture was concentrated under reduced pressure to remove solvent. To the residue was added saturated NaHCO₃ aqueous solution to adjust the pH-7 and extracted with ethyl acetate (2×3 mL). The combined organic layers were dried over anhydrous sodium sulfate, concentrated and purified by prep-HPLC (column: Welch Ultimate C18 150*25 mm*5 um; mobile phase: [water (FA)-ACN]; B %: 10%-40%, 10 min) to afford the title compound (7.30 mg, 26% yield) as off-white solid. 1H NMR (400 MHz, METHANOL-d4) δ=7.57-7.46 (m, 1H), 7.15 (br t, J=9.6 Hz, 1H), 6.99 (br d, J=5.2 Hz, 2H), 6.90 (s, 1H), 6.71 (dd, J=11.2, 17.6 Hz, 1H), 6.04 (d, J=17.6 Hz, 1H), 5.57-5.33 (m, 2H), 4.83-4.75 (m, 2H), 4.43-4.19 (m, 5H), 4.13 (br d, J=17.6 Hz, 1H), 3.89-3.78 (m, 1H), 3.72 (br d, J=17.6 Hz, 1H), 3.58 (br d, J=18.4 Hz, 4H), 3.38 (br d, J=4.8 Hz, 2H), 3.24 (br d, J=8.4 Hz, 3H), 2.77 (br d, J=12.4 Hz, 1H), 2.55-2.34 (m, 2H), 2.32-2.13 (m, 3H), 2.10-1.98 (m, 1H), 1.12 (br t, J=7.2 Hz, 3H); LCMS (ESI, M+1): m/z=628.5.

### Example 249

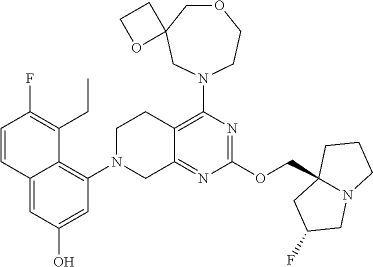 [SMILES: CCc1c(F)ccc2cc(O)cc(N3CCc4c(nc(OC[C@@]56CCCN5C[C@H](F)C6)nc4N4CCOCC5(CCO5)C4)C3)c12]

### 5-ethyl-6-fluoro-4-(2-(((2R,7aS)-2-fluorohexahydro-1H-pyrrolizin-7a-yl)methoxy)-4-(1,6-dioxa-9-azaspiro[3.6]decan-9-yl)-5,6-dihydropyrido[3,4-d]pyrimidin-7(8H)-yl)naphthalen-2-ol

Synthesized according to Example 248 except that TFA instead of HCl was used in step C. The title compound was obtained as yellow solid (FA salt). 1H NMR (400 MHz, METHANOL-d4) δ=8.54 (s, 1H), 7.50 (dd, J=5.9, 8.8 Hz, 1H), 7.13 (t, J=9.2 Hz, 1H), 7.01-6.93 (m, 2H), 5.37-5.19 (m, 1H), 4.59 (br s, 2H), 4.52-4.42 (m, 2H), 4.17-3.96 (m, 6H), 3.95-3.74 (m, 4H), 3.70-3.45 (m, 3H), 3.42-3.34 (m, 2H), 3.26-3.21 (m, 2H), 3.17 (br s, 1H), 3.05-2.96 (m, 1H), 2.71-2.61 (m, 1H), 2.59-2.48 (m, 1H), 2.34-2.15 (m, 2H), 2.14-2.05 (m, 1H), 2.02-1.93 (m, 2H), 1.89-1.78 (m, 1H), 1.12-1.03 (m, 3H). LCMS (ESI, M+1): m/z=622.5.

### Example 250

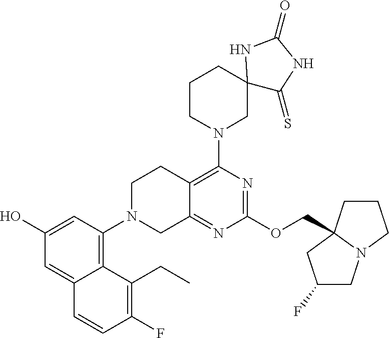 [SMILES: CCc1c(F)ccc2cc(O)cc(N3CCc4c(nc(OC[C@@]56CCCN5C[C@H](F)C6)nc4N4CCCC5(C4)NC(=O)NC5=S)C3)c12]

### 7-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-4-thioxo-1,3,7-triazaspiro[4.5]decan-2-one

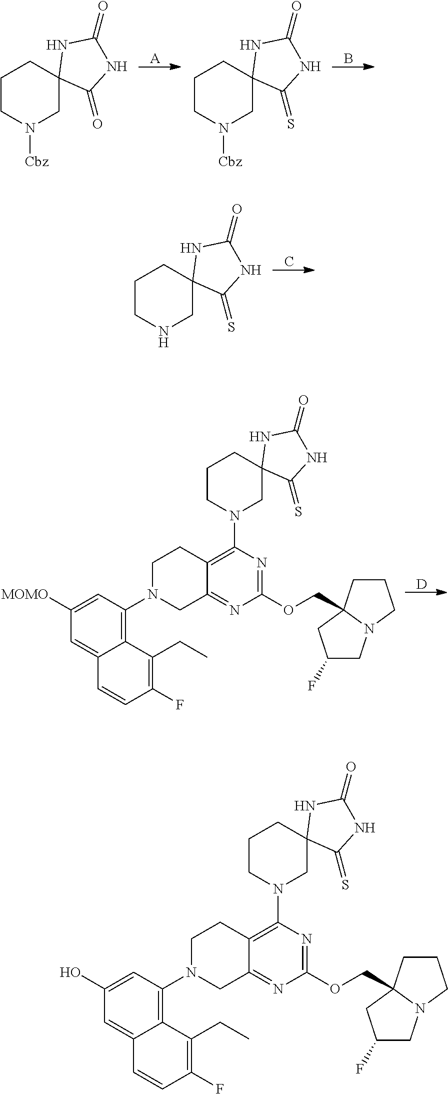

Step A. benzyl 2-oxo-4-thioxo-1,3,7-triazaspiro[4.5]decane-7-carboxylate: To a solution of benzyl 2,4-dioxo-1,3,7-triazaspiro[4.5]decane-7-carboxylate (1.00 g, 1.0 equiv) in THF (10 mL) was added Lawessons reagent (4.00 g, 3.0 equiv). The reaction was stirred at 60° C. for 6 hours. After completion, the reaction was concentrated under reduced pressure to remove solvent. To the residue was added MeOH (30 mL) and filtered to give a filter cake. The filter cake was washed with EtOAc (30 mL) to afford the title compound (360 mg, 34% yield) as white solid; 1H NMR (400 MHz, DMSO-d6) δ=12.54 (br s, 1H), 10.73 (s, 1H), 9.25 (br s, 1H), 8.70-8.35 (m, 1H), 7.59-7.10 (m, 5H), 5.06 (br s, 2H), 4.17-3.63 (m, 2H), 3.27-2.71 (m, 2H), 2.15-1.88 (m, 1H), 1.83-1.50 (m, 3H).

Step B. 4-thioxo-1,3,7-triazaspiro[4.5]decan-2-one: To a solution of benzyl 2-oxo-4-thioxo-1,3,7-triazaspiro[4.5]decane-7-carboxylate (400 mg, 1.0 equiv) in DCM (4 mL) was added trimethylsilyl iodide (1.47 g, 5.9 equiv) at 0° C. The reaction was stirred at 0-20° C. for 0.5 hours. The mixture was diluted with H₂O (10 mL) and washed with DCM (10×10 mL). The water phase was lyophilized to afford the title compound (400 mg, crude) as yellow solid; LCMS (ESI, M+1): m/z=186.0.

The last two steps were performed according to Example 248. The title compound was obtained as white solid (FA salt). 1H NMR (400 MHz, METHANOL-d4) 8=7.52 (dd, J=5.6, 9.2 Hz, 1H), 7.15 (t, J=9.2 Hz, 1H), 7.03-6.92 (m, 2H), 5.52-5.32 (m, 1H), 4.45-4.22 (m, 4H), 4.18-4.05 (m, 2H), 3.71-3.58 (m, 2H), 3.56-3.38 (m, 5H), 3.22-3.14 (m, 3H), 3.07-2.93 (m, 1H), 2.84-2.68 (m, 1H), 2.40-2.23 (m, 3H), 2.19-2.10 (m, 2H), 2.09-1.98 (m, 2H), 1.95-1.77 (m, 3H), 1.17-1.05 (m, 3H); LCMS (ESI, M+1): m/z=664.6.

### Example 251

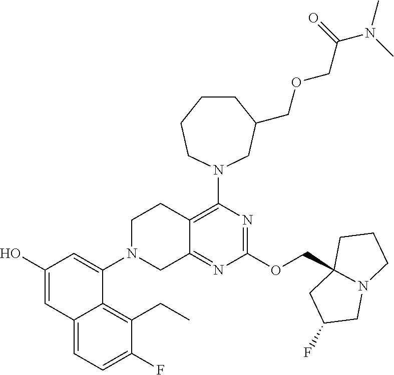 [SMILES: CCc1c(F)ccc2cc(O)cc(N3CCc4c(nc(OC[C@@]56CCCN5C[C@H](F)C6)nc4N4CCCCC(COCC(=O)N(C)C)C4)C3)c12]

### 2-((1-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)azepan-3-yl)methoxy)-N,N-dimethylacetamide

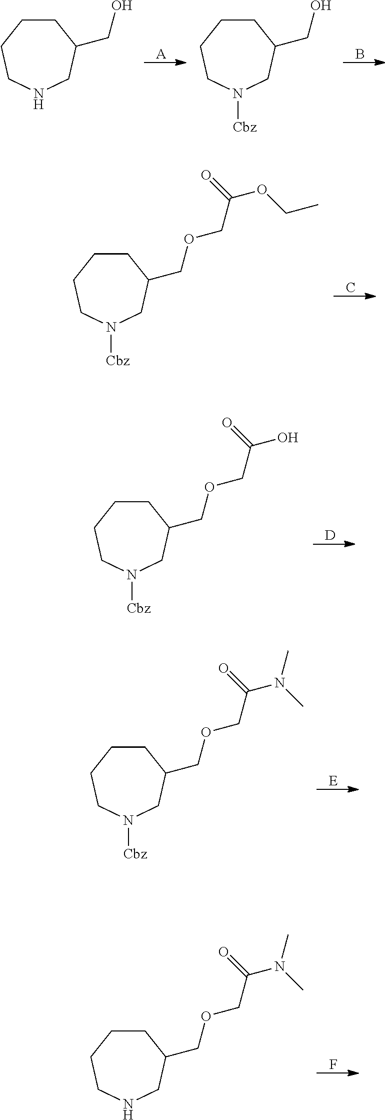

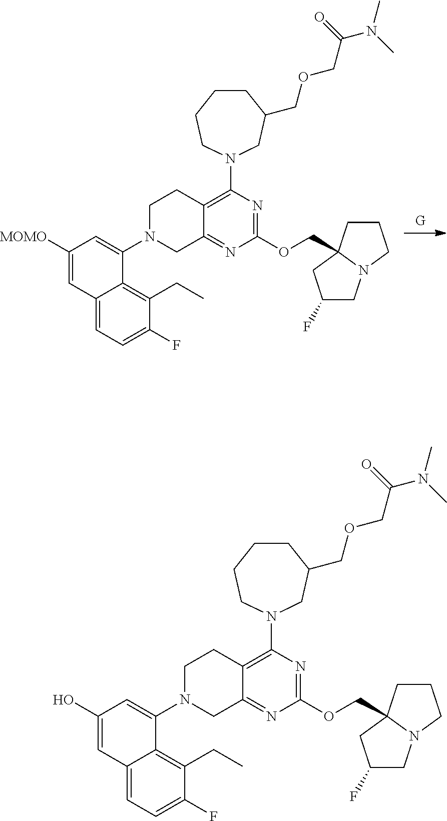

Step A. benzyl 3-(hydroxymethyl)azepane-1-carboxylate: To a mixture of azepan-3-ylmethanol (4.00 g, 1.0 equiv) and K2CO3 (12.8 g, 3.0 equiv) in THF (40 mL) and water (20 mL) was added CbzCl (10.5 g, 2.0 equiv) dropwise at 0° C., the reaction was stirred at 0-10° C. for 17 hours. The mixture was extracted with ethyl acetate (30 mL×3). The organic layers were dried over Na₂SO₄, filtered, concentrated, and purified by column chromatography to afford the title compound (7 g, 55% yield) as yellow solid.

Step B. benzyl 3-[(2-ethoxy-2-oxo-ethoxy)methyl]azepane-1-carboxylate: To a solution of benzyl 3-(hydroxymethyl)azepane-1-carboxylate (1.00 g, 1.0 equiv) in THF (15 mL) was added NaH (303 mg, 60% purity, 2.0 equiv). The reaction was stirred at 0° C. for 0.5 hours and then ethyl 2-bromoacetate (761 mg, 1.2 equiv) was added. The reaction was stirred at 15° C. for 6 hours. The mixture was quenched with water (50 mL) at 0° C. and extracted with ethyl acetate (20 mL×3). The organic layers were dried over Na₂SO₄, filtered, concentrated, and purified by column chromatography (SiO2, petroleum ether/ethyl acetate=10/1 to 2/1) to afford the title compound (630 mg, 17% yield) as yellow solid.

Step C. 2-[(1-benzyloxycarbonylazepan-3-yl)methoxy]acetic acid: A mixture of benzyl 3-[(2-ethoxy-2-oxo-ethoxy)methyl]azepane-1-carboxylate (500 mg, 1.0 equiv) in a solution of LiOH (2 M, 10.00 mL, 13.9 equiv) was stirred at 15° C. for 1 hour. The mixture was acidified with HCl (1 M, 20 mL) and extracted with ethyl acetate (30 mL×3). The combined organic layers were dried, filtered and concentrated to afford the title compound (560 mg, 84% yield) as yellow solid.

Step D benzyl 3-[[2-(dimethylamino)-2-oxo-ethoxy]methyl]azepane-1-carboxylate: A mixture of 2-[(1-benzyloxycarbonylazepan-3-yl)methoxy]acetic acid (165 mg, 1.0 equiv), N,N-diethylpropan-2-amine (199 mg, 3.0 equiv) and HATU (292 mg, 1.5 equiv) in DCM (2 mL) was stirred at 30° C. for 2 hours and then dimethylamine hydrochloride (50.2 mg, 1.2 equiv) was added. The reaction was stirred at 30° C. for 2 hours. The mixture was concentrated to afford the title compound (560 mg, 84% yield) as white solid.

Step E 2-(azepan-3-ylmethoxy)-N,N-dimethyl-acetamide: A mixture of benzyl 3-[[2-(dimethylamino)-2-oxo-ethoxy]methyl]azepane-1-carboxylate (180 mg, 1.0 equiv) and Pd/C (15 mg, 60% purity, 0.1 equiv) in MeOH (5 mL) was stirred at 20° C. for 2 hours under H2 atmosphere (15 Psi). The mixture was filtered and concentrated to afford the title compound (200 mg, 54% yield) as white solid.

The last two steps were performed according to Example 248. The title compound was obtained as brown solid. 1H NMR (400 MHz, DMSO-d6) δ=9.68 (br d, J=4.4 Hz, 1H), 7.59 (dd, J=6.0, 8.8 Hz, 1H), 7.23 (t, J=9.6 Hz, 1H), 6.98 (d, J=4.0 Hz, 2H), 5.33-5.14 (m, 1H), 4.10 (br d, J=4.4 Hz, 3H), 3.98-3.74 (m, 4H), 3.69-3.44 (m, 2H), 3.30 (s, 6H), 3.13-3.01 (m, 4H), 2.97 (br s, 1H), 2.90 (d, J=7.6 Hz, 3H), 2.78 (d, J=3.6 Hz, 4H), 2.67 (br s, 1H), 2.33 (s, 1H), 2.12-1.90 (m, 4H), 1.84-1.62 (m, 6H), 1.35-1.23 (m, 2H), 1.03 (q, J=7.2 Hz, 3H); LCMS (ESI, M+1): m/z=693.3.

### Example 252

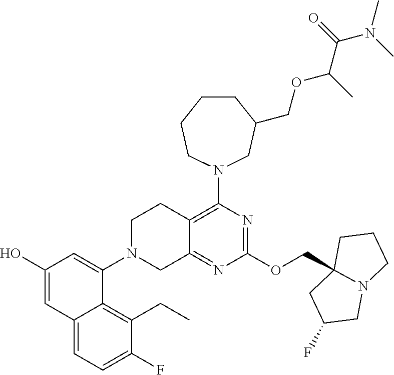 [SMILES: CCc1c(F)ccc2cc(O)cc(N3CCc4c(nc(OC[C@@]56CCCN5C[C@H](F)C6)nc4N4CCCCC(COC(C)C(=O)N(C)C)C4)C3)c12]

### 2-((1-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)azepan-3-yl)methoxy)-N,N-dimethylpropanamide

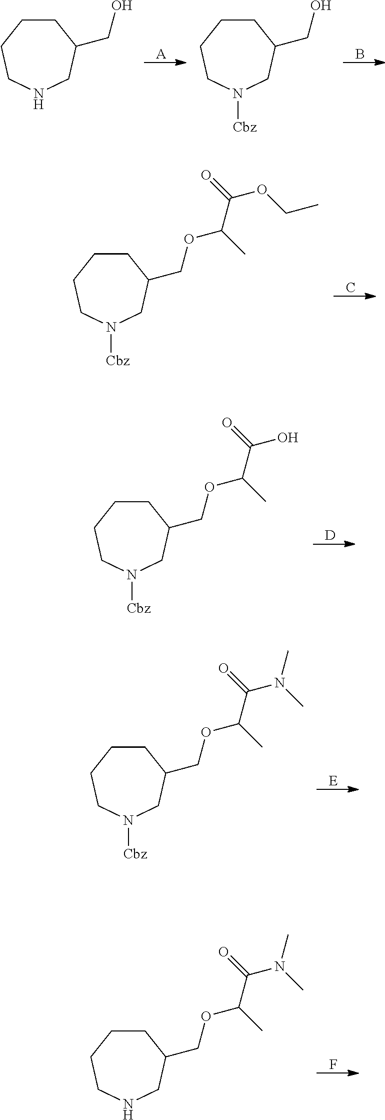

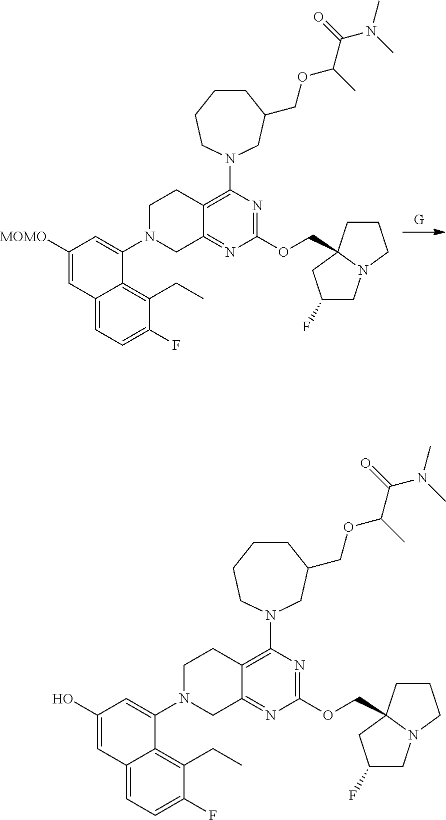

Synthesized according to Example 251. The title compound was obtained as white solid. 1H NMVR (400 MHtVz, METHANOL-d4) δ=7.50 (dd, J=6.0, 8.8 Hz, 1H), 7.13 (t, J=9.2 Hz, 1H), 7.02-6.89 (m, 2H), 5.43-5.10 (mn, 1H), 4.49-3.95 (mn, 6H), 3.75-3.57 (mn, 1H), 3.56-3.32 (m, 5H), 3.30-3.13 (mn, 7H), 3.10 (dd, J=1.2, 6.0 Hz, 3H), 3.03-2.96 (mn, 1H), 2.94-2.88 (mn, 3H), 2.85-2.66 (mn, 1H), 2.39-2.25 (mn, 1H), 2.23-1.63 (mn, 10H), 1.42 (br dd, J=5.6, 8.8 Hz, 2H), 1.34-1.26 (mn, 3H1), 1.17-1.03 (mn, 3H); LCMS (ESI, M+1). m/z=707.5.

### Example 253

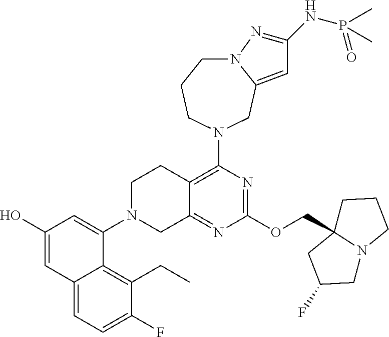 [SMILES: CCc1c(F)ccc2cc(O)cc(N3CCc4c(nc(OC[C@@]56CCCN5C[C@H](F)C6)nc4N4CCCn5nc(NP(C)(C)=O)cc5C4)C3)c12]

### N-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-P,P-dimethylphosphinic amide

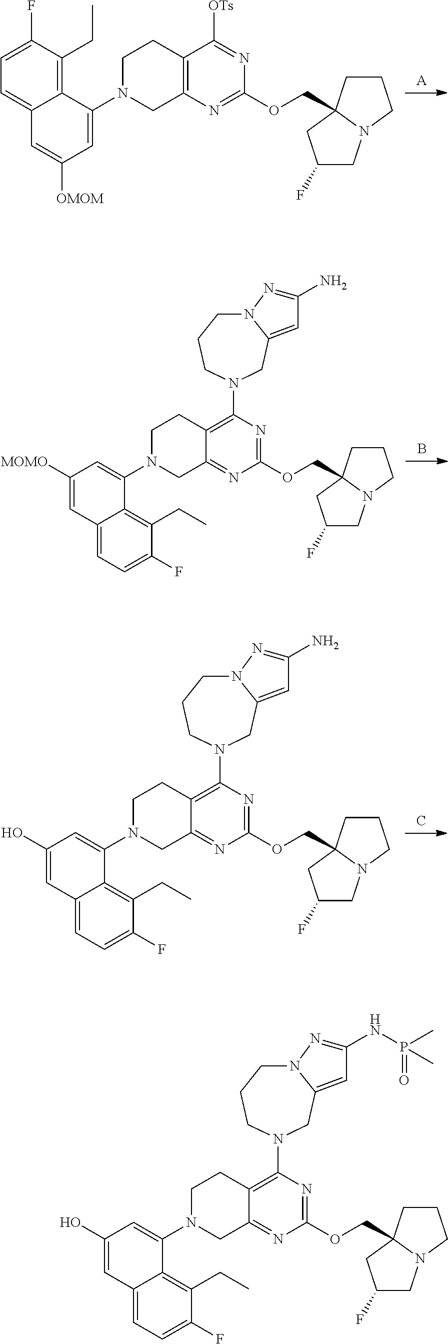

Step A. 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-amine: To a mixture of 7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (7.50 g, 1.0 equiv) and 5,6,7,8-tetrahydro-4H-pyrazolo[1,5-a][1,4]diazepin-2-amine (2.46 g, 1.5 equiv) in DMF (5.0 mL) were added N,N-diethylpropan-2-amine (8.37 g, 6.0 equiv) and 4A molecular sieve (2.00 g). The reaction was stirred at 40° C. for 6 hours. The mixture was diluted with water (20 mL) and extracted with EtOAc (3×30 mL). The combined organic layers were washed with brine (100 mL), dried over anhydrous sodium sulfate, and concentrated to afford the title compound (7.20 g, 98% yield) as yellow solid; LCMS (ESI, M+1): m/z=675.6.

Step B. 4-(4-(2-amino-7,8-dihydro-4H-pyrazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol: To a mixture of 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-amine (100 mg, 1.0 equiv) in ACN (0.5 mL) was added HCl-MeOH (1.0 mL, 27 equiv). The reaction was stirred at 20° C. for 0.5 hours. The mixture was concentrated and purified by reversed phase flash chromatography [C18, 0.1% formic acid condition] to afford the title compound (78 mg, 83% yield) as orange solid; LCMS (ESI, M+1): m/z=631.4.

Step C. N-(5-(7-(8-ethyl-7-fluoro-3-hydroxynaphthalen-1-yl)-2-(((2R,7aS)-2-fluorohexahydro-1H-pyrrolizin-7a-yllmethoxy)-5,6,7,8-tetrahydropyrido[3,4-d]pyrimidin-4-yl-5,6,7,8-tetrahydro-4H-pyrazolo[1,5-a][1,4]diazepin-2-yl)-P,P-dimethylphosphinic amide: To a mixture of 4-(4-(2-amino-7,8-dihydro-4H-pyrazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol (78.0 mg, 1.0 equiv) in Pyridine (784 mg, 80 equiv) were added dimethylphosphinic chloride (34.8 mg, 2.5 equiv) and THE (0.8 mL). The reaction was stirred at 20° C. for 2 hours. The mixture was concentrated and purified by prep-HPLC [Phenomenex C18 150×25 mm×10 μm; A: water (NH₄HCO₃), B: ACN; B %: 28%-58% over 8 min] and lyophilized to afford the title compound (5.59 mg, 7.3% yield) as off-white solid. 1H NMR (400 MHz, dimethylsulfoxide-d6) δ=9.73 (br s, 1H), 7.66-7.54 (m, 1H), 7.39 (d, J=7.2 Hz, 1H), 7.24 (t, J=9.6 Hz, 1H), 7.06-6.91 (m, 2H), 5.68 (s, 1H), 5.35-5.16 (m, 1H), 4.87 (br d, J=16.4 Hz, 1H), 4.61 (br d, J=16.0 Hz, 1H), 4.26 (br d, J=4.8 Hz, 2H), 4.05 (br dd, J=2.8, 13.2 Hz, 1H), 3.96-3.72 (m, 4H), 3.62 (br d, J=17.6 Hz, 1H), 3.14-2.95 (m, 6H), 2.84-2.76 (m, 1H), 2.70-2.57 (m, 2H), 2.20-2.06 (m, 2H), 2.05-1.88 (m, 4H), 1.87-1.79 (m, 1H), 1.78-1.69 (m, 2H), 1.48 (d, J=14.4 Hz, 6H), 1.08 (t, J=7.2 Hz, 3H); 19F NMR (400 MHz, dimethylsulfoxide-d6) δ=−121.333, -171.984; LCMS (ESI, M+1): m/z=707.5.

### Example 254

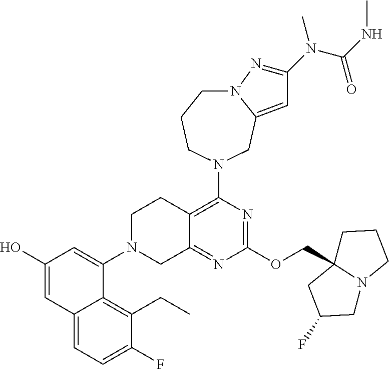 [SMILES: CCc1c(F)ccc2cc(O)cc(N3CCc4c(nc(OC[C@@]56CCCN5C[C@H](F)C6)nc4N4CCCn5nc(N(C)C(=O)NC)cc5C4)C3)c12]

### 1-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-1,3-dimethylurea

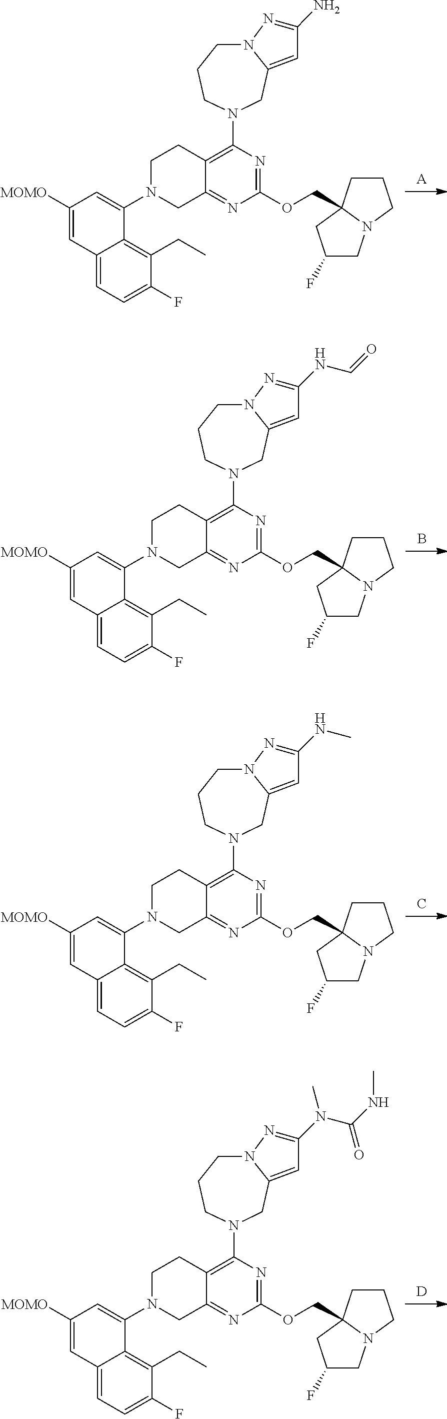

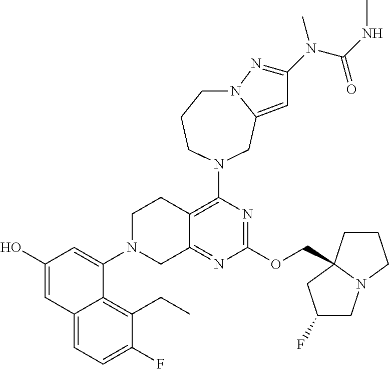 [SMILES: CCc1c(F)ccc2cc(O)cc(N3CCc4c(nc(OC[C@@]56CCCN5C[C@H](F)C6)nc4N4CCCn5nc(N(C)C(=O)NC)cc5C4)C3)c12]

Step A. N-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-5,6,7,8-tetrahy dro-4H-pyrayzolo[1,5-a][1,4]diazepin-2-yl)formamide: To a flask containing 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-amine (1.00 g, 1.0 equiv) were added ethyl formate (2.76 g, 25.2 equiv) and formamide (3.39 g, 50.8 equiv). The reaction was stirred at 90° C. for 5 hours. The mixture was concentrated and triturated with H₂O (50 mL) to afford the title compound (0.80 g, 77% yield) as yellow solid; LCMS (ESI, M+1): m/z=703.4.

Step B. 5-(7-(8-ethyl-7-fluoro-3-(methoxymethoxy)naphthalen-1-yl)-2-(((2R,7aS)-2-fluorotetrahydro-1H-pyrrolizin-7a-yl)methoxy)-5,6,7,8-tetrahydropyrido[3,4-d] pyrimidin-4-yl)-N-methyl-5,6,7,8-tetrahydro-4H-pyrazolo[1,5-a][1,4]diazepin-2-amine: To a mixture of N-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)formamide (0.30 g, 1.0 equiv) in THF (3.0 mL) was added DIBAL-H (1 M, 1.28 mL, 3.0 equiv) at 0° C. under nitrogen. The reaction was stirred at −78° C. for 3 hours. The mixture was diluted with saturated NH₄Cl aqueous solution (10 mL) and extracted with EtOAc (3×30 mL). The combined organic layers were washed with brine (100 mL), dried over anhydrous sodium sulfate, concentrated, and purified by reversed phase chromatography [C18, 0.1% formic acid condition] to afford the title compound (0.10 g, 34% yield) as yellow solid. LCMS (ESI, M+1): m/z=689.2.

Step C. 1-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-5,6,7,8-tetrahydro-4H-pyrazolo[1,5-a][1,4]diazepin-2-yl)-1,3-dimethylurea: To a mixture of 5-(7-(8-ethyl-7-fluoro-3-(methoxymethoxy)naphthalen-1-yl)-2-(((2R,7aS)-2-fluorotetrahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepin-2-amine (100 mg, 1.0 equiv) in THF (0.5 mL) were added pyridine (490 mg, 42.7 equiv) and methylcarbamic chloride (33.9 mg, 2.5 equiv). The reaction was stirred at 20° C. for 2 hours. The mixture was concentrated and purified by reversed phase chromatography [C18, 0.1% formic acid condition] to afford the title compound (100 mg, 89% yield) as yellow oil; LCMS (ESI, M+1): m/z=746.7.

Step D. 1-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-1,3-dimethylurea: To a mixture of 1-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-1,3-dimethylurea (90.0 mg, 1.0 equiv) in ACN (220 μL) was added HCl-MeOH (4 M, 452 μL, 15 equiv). The reaction was stirred at 20° C. for 1 hour. The mixture was concentrated, dissolved in methanol (1 mL), neutralized with solid NaHCO₃, filtered, purified by prep-HPLC [Phenomenex luna 150×25 mm×10 μm; A: water (FA), B: ACN; B %: 19%-49% over 10 min] and lyophilized to give a residue. The residue was triturated with acetonitrile (2 mL) at 20° C. for 15 minutes, purified by prep-HPLC [Welch Ultimate C18 150 x 25 mm×5 m; A: water (FA), B: ACN; B %: 18%-48% over 10 min] and lyophilized to afford the title compound (11.8 mg, 14% yield, 0.24 equvi FA) as off-white solid. 1H NMR (400 MHz, dimethylsulfoxide-d6) δ=9.79-9.62 (m, 1H), 7.87-7.77 (m, 1H), 7.61-7.55 (m, 1H), 7.28-7.18 (m, 1H), 7.02-6.95 (m, 2H), 6.07-6.00 (m, 1H), 5.34-5.11 (m, 1H), 4.96-4.83 (m, 1H), 4.77-4.65 (m, 1H), 4.41-4.29 (m, 2H), 4.12-4.00 (m, 1H), 3.94-3.76 (m, 4H), 3.65-3.57 (m, 1H), 3.18-3.08 (m, 6H), 3.07-2.90 (m, 4H), 2.84-2.76 (m, 1H), 2.74-2.64 (m, 4H), 2.45-2.35 (m, 1H), 2.24-2.11 (m, 1H), 2.09-2.01 (m, 1H), 2.00-1.88 (m, 3H), 1.86-1.77 (m, 1H), 1.77-1.62 (m, 2H), 1.10-1.00 (m, 3H); 19F NMR (400 MHz, dimethylsulfoxide-d6) δ=−121.341, -172.090; LCMS (ESI, M+1): m/z =702.5.

### Example 255

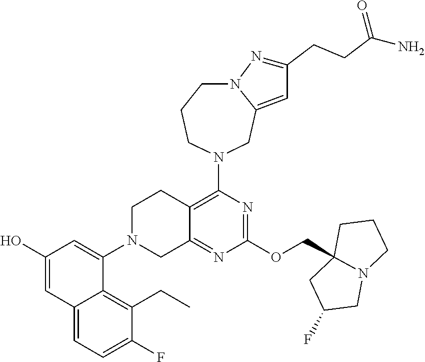 [SMILES: CCc1c(F)ccc2cc(O)cc(N3CCc4c(nc(OC[C@@]56CCCN5C[C@H](F)C6)nc4N4CCCn5nc(CCC(N)=O)cc5C4)C3)c12]

### 3-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)propanamide

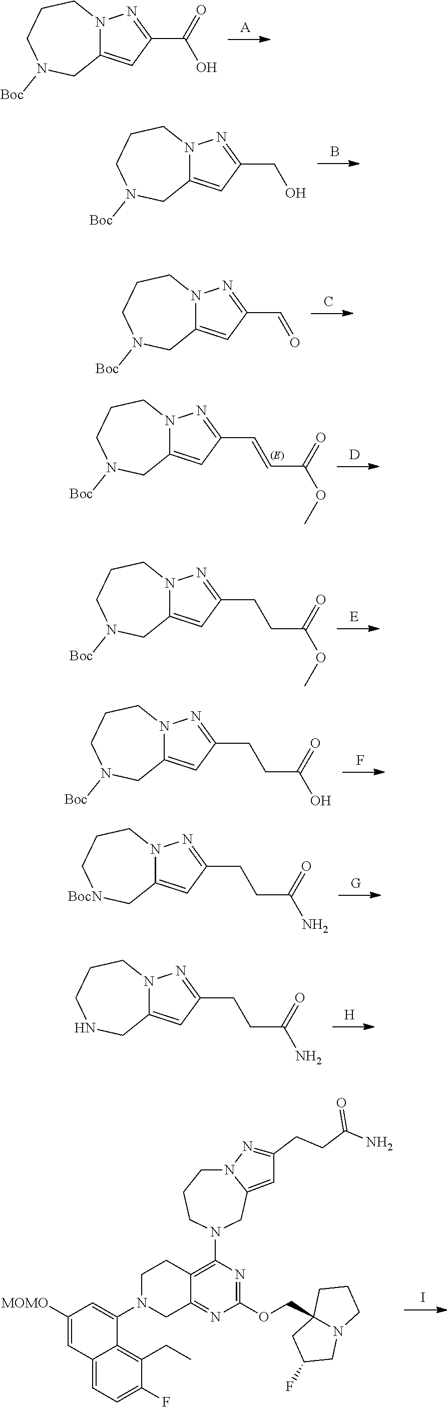

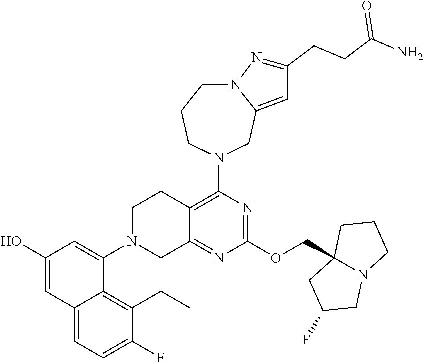 [SMILES: CCc1c(F)ccc2cc(O)cc(N3CCc4c(nc(OC[C@@]56CCCN5C[C@H](F)C6)nc4N4CCCn5nc(CCC(N)=O)cc5C4)C3)c12]

Step A. tert-butyl 2-(hydroxymethyl)-7,8-dihydro-4H-pyrazolo[1,5-a][1,4]diazepine-5(6H)-carboxylate: To a solution of 5-(tert-butoxycarbonyl)-5,6,7,8-tetrahydro-4H-pyrazolo[1,5-a][1,4]diazepine-2-carboxylic acid (8.5 g, 1.0 equiv) in THF (150 mL) was added BH3 THF (1.0 M, 121 mL, 4.0 equiv) at 0° C. under N2. The reaction was stirred at 60° C. for 12 hours under N2 atmosphere. The reaction was quenched with MeOH (200 mL) at 0° C. The mixture was concentrated and purified with prep-HPLC (Column: 120 g Flash Column WelchUltimate XB_C18 20-40 μm; 120 A; Mobile phase MeCN/H₂O; Flow rate 85 mL/min ; Gradient B % 5-40% 20 min;40% 5 min) to afford the title compound (5.7 g, 70.6% yield, N/A purity) as colorless oil. 1H NMR (400 MHz, DMSO-d6) 8=8.13 (s, 1H), 6.06 (s, 1H), 4.92 (br s, 1H), 4.50-4.23 (m, 6H), 3.62 (br s, 2H), 1.71 (br s, 2H), 1.34 (br s, 9H). LCMS (ESI, M+1): m/z =268.2.

Step B. tert-butyl 2-formyl-7,8-dihydro-4H-pyrazolo[1,5-a][1,4]diazepine-5(6H)-carboxylate: To a solution of tert-butyl 2-(hydroxymethyl)-7,8-dihydro-4H-pyrazolo[1,5-a][1,4]diazepine-5(6H)-carboxylate (1.0 g, 1.0 equiv) in DCM (10 mL) was added Dess-Martin reagent (2.38 g, 1.5 equiv). The reaction was stirred at 25° C. for 2 hours. The mixture was filtered and purified by column chromatography (SiO2, Petroleum ether/Ethyl acetate=5/1 to 1/1) to afford the title compound (800 mg, 80.6% yield) as white solid. LCMS (ESI, M+1): m/z=266.2.

Step C. (E)-tert-butyl 2-(3-methoxy-3-oxoprop-1-en-1-yl)-7,8-dihydro-4H-pyrazolo[1,5-a][1,4]diazepine-5(6H)-carboxylate: To a solution of ethyl 2-(diethoxyphosphoryl)acetate (659 mg, 1.2 equiv) in THE (10.0 mL) was added NaH (181 mg, 60% purity, 1.5 equiv) and the mixture was stirred for 0.5 hours at 0° C. Tert-butyl 2-formyl-7,8-dihydro-4H-pyrazolo[1,5-a][1,4]diazepine-5(6H)-carboxylate (800 mg, 3.02 mmol, 1.0 equiv) was added at 0° C. The reaction was stirred at 25° C. for 2.5 hours. The mixture was quenched with NH₄Cl.aq (50 mL) at 0° C. and extracted with ethyl acetate (50 mL×3). The combined organic layers were washed with brine (50 mL), dried over Na₂SO₄, filtered, and purified by column chromatography (SiO2, Petroleum ether/Ethyl acetate=5/1 to 1/1) to afford the title compound (700 mg, 72.2% yield) as white solid. LCMS (ESI, M+1): m/z=322.1

Step D. tert-butyl 2-(3-methoxy-3-oxopropyl)-7,8-dihydro-4H-pyrazolo[1,5-a][1,4]diazepine-5(6H)-carboxylate: To a solution of (E)-tert-butyl 2-(3-methoxy-3-oxoprop-1-en-1-yl)-7,8-dihydro-4H-pyrazolo[1,5-a][1,4]diazepine-5(6H)-carboxylate (700 mg, 1.0 equiv) in EtOH (10.0 mL) was added Pd/C (100 mg, 10% purity) under N2 atmosphere. The suspension was degassed and purged with H2 3 times. The reaction was stirred under H2 (15 Psi) at 20° C. for 1 hour. The mixture was filtered and concentrated to afford the title compound (600 mg, crude) as yellow oil. LCMS (ESI, M+1): m/z=324.3

Step E. 3-(5-(tert-butoxycarbonyl)-5,6,7,8-tetrahydro-4H-pyrazolo[1,5-a][1,4]diazepin-2-yl)propanoic acid: To a solution of tert-butyl 2-(3-methoxy-3-oxopropyl)-7,8-dihydro-4H-pyrazolo[1,5-a][1,4]diazepine-5(6H)-carboxylate (600 mg, 1.0 equiv) in MeOH (10.0 mL) and water (2.0 mL) was added NaOH (223 mg, 3.0 equiv). The reaction was stirred at 60° C. for 2 hours. The mixture was quenched with citric acid (2 M, 10 mL) at 20° C. and then extracted with dichloromethane (3×10 mL). The combined organic layers were washed with brine (10 mL), dried over Na₂SO₄, filtered, and concentrated to afford the title compound (700 mg, crude) as yellow oil. LCMS (ESI, M+1): m/z=310.3

Step F. tert-butyl 2-(3-amino-3-oxopropyl)-7,8-dihydro-4H-pyrazolo[1,5-a][1,4]diazepine-5(6H)-carboxylate: To a solution of 3-(5-(tert-butoxycarbonyl)-5,6,7,8-tetrahydro-4H-pyrazolo[1,5-a][1,4]diazepin-2-yl)propanoic acid (300 mg, 1.0 equiv) in THE (6.0 mL) were added HATU (553 mg, 1.5 equiv), N,N-diethylpropan-2-amine (376 mg, 3.0 equiv) and NH₄Cl (77.8 mg, 1.5 equiv). The mixture was stirred at 20° C. for 2 hours. The reaction was diluted with water (20 mL) and extracted with DCM (3×20 mL). The combined organic layers were washed with brine (20 mL×3), dried over Na₂SO₄, concentrated, and purified with prep-HPLC [column: Phenomenex luna C18 150×40 mm×15 μm; mobile phase: [water (FA)-ACN]; B %: 15%-45%, 10 minutes] to afford the title compound (100 mg, 33% yield) as white solid. LCMS (ESI, M+1): m/z=309.1

Step G. 3-(5,6,7,8-tetrahydro-4H-pyrazolo[1,5-a][1,4]diazepin-2-yl)propanamide: To a solution of tert-butyl 2-(3-amino-3-oxopropyl)-7,8-dihydro-4H-pyrazolo[1,5-a][1,4]diazepine-5(6H)-carboxylate (500 mg, 1.0 equiv) in DCM (2.0 mL) was added TFA (2.00 mL). The mixture was stirred at 20° C. for 1 hour. The reaction was concentrated to afford the title compound (300 mg, crude) as yellow oil. LCMS (ESI, M+1): m/z=209.1

The last two steps were performed according to Example 248. The title compound was obtained as yellow solid (FA salt). 1H NMR (400 MHz, METHANOL-d4) δ=8.53 (s, 1H), 7.51 (dd, J=6.0, 8.8 Hz, 1H), 7.15 (t, J=9.2 Hz, 1H), 7.03-6.92 (m, 2H), 6.07 (s, 1H), 5.41-5.21 (m, 1H), 4.78-4.74 (m, 1H), 4.41 (br s, 2H), 4.21-4.16 (m, 1H), 4.13-4.09 (m, 2H), 4.08-3.95 (m, 2H), 3.70 (br d, J=18.0 Hz, 1H), 3.50 (br s, 1H), 3.44-3.34 (m, 3H), 3.28 (br s, 1H), 3.17 (br d, J=8.4 Hz, 2H), 3.07 (br d, J=5.6 Hz, 1H), 2.87-2.81 (m, 2H), 2.70 (br d, J=12.8 Hz, 1H), 2.51 (t, J=7.6 Hz, 2H), 1.99 (br d, J=6.4 Hz, 8H), 1.96-1.85 (m, 1H), 1.12 (br t, J=6.8 Hz, 3H); LCMS (ESI, M+1): m/z=687.5.

### Example 256

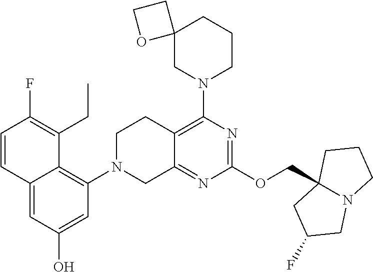 [SMILES: CCc1c(F)ccc2cc(O)cc(N3CCc4c(nc(OC[C@@]56CCCN5C[C@H](F)C6)nc4N4CCCC5(CCO5)C4)C3)c12]

### 5-ethyl-6-fluoro-4-(2-(((2R,7aS)-2-fluorohexahydro-1H-pyrrolizin-7a-yl)methoxy)-4-(1-oxa-6-azaspiro[3.5]nonan-6-yl)-5,6-dihydropyrido[3,4-d]pyrimidin-7(8H)-yl)naphthalen-2-ol

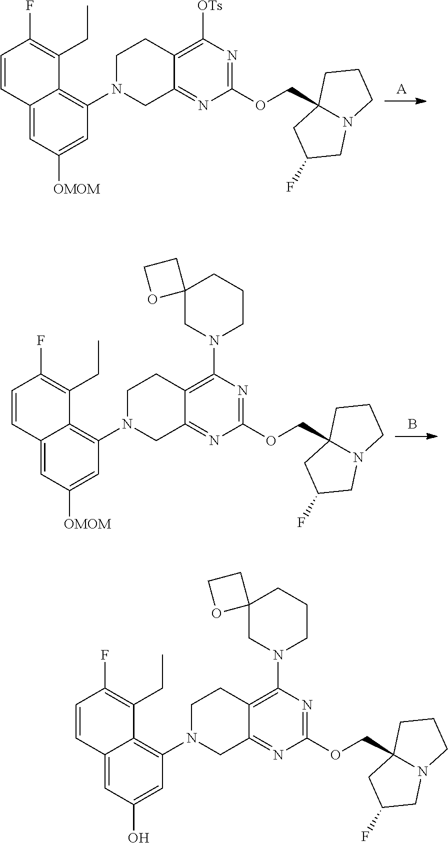

Synthesized according to Example 248 except that TFA instead of HCl was used in the last step. The title compound was obtained as yellow solid. LCMS (ESI, M+1): m/z=606.5

### Example 257

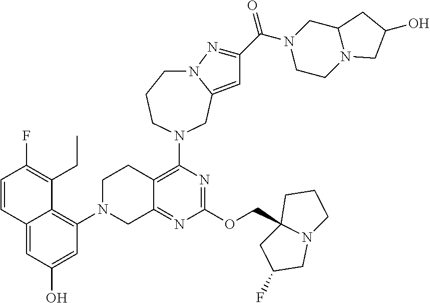 [SMILES: CCc1c(F)ccc2cc(O)cc(N3CCc4c(nc(OC[C@@]56CCCN5C[C@H](F)C6)nc4N4CCCn5nc(C(=O)N6CCN7CC(O)CC7C6)cc5C4)C3)c12]

### (5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(7-hydroxyhexahydropyrrolo[1,2-a]pyrazin-2(1H)-yl)methanone

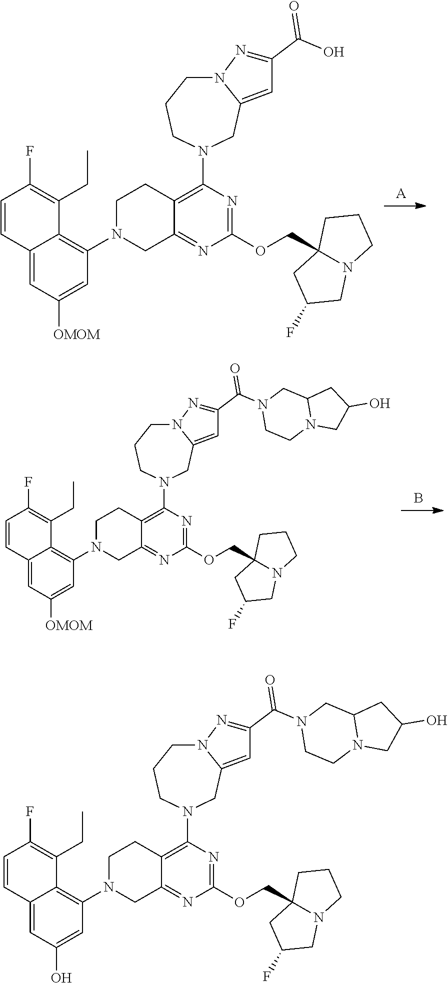

Synthesized according to Example 233 except that HCl instead of TFA was used in the last step. The title compound was obtained as yellow solid. 1HNMR (400 MHz, METHANOL-d4) δ=7.51 (dd, J=8.8, 5.6 Hz, 1H) 7.14 (t, J=9.2 Hz, 1H) 6.96 (s, 2H) 6.58-6.60 (m, 1H) 5.17-5.35 (m, 1H) 5.00 (br d, J=16.8 Hz, 1H) 4.77-4.84 (m, 1H) 4.71 (br s, 1H) 4.59 (br s, 1H) 4.53 (br d, J=5.2 Hz, 2H) 4.34 (br s, 1H) 3.99-4.14 (m, 4H) 3.62-3.71 (m, 1H) 3.50 (br d, J=17.6 Hz, 1H) 3.35-3.45 (m, 3H) 3.13-3.26 (m, 5H) 3.08 (br s, 1H) 2.94-3.03 (m, 3H) 2.72 (br d, J=13.2 Hz, H) 2.23-2.41 (m, 4H) 2.12-2.22 (m, 3H) 2.07 (br d, J=9.2 Hz, 3H) 1.82-2.01 (m, 4H) 1.10 (br t, J=6.8 Hz, 3H); LCMS (ESI, M+1). m/z=784.4.

### Example 258

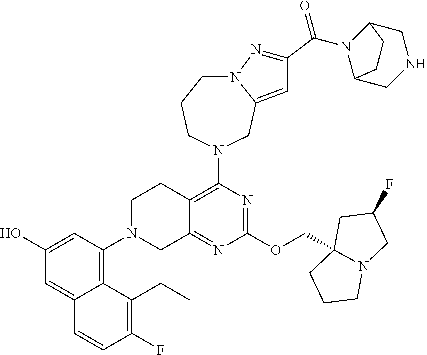 [SMILES: CCc1c(F)ccc2cc(O)cc(N3CCc4c(nc(OC[C@@]56CCCN5C[C@H](F)C6)nc4N4CCCn5nc(C(=O)N6C7CCC6CNC7)cc5C4)C3)c12]

### 3,8-diazabicyclo[3.2.1]octan-8-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

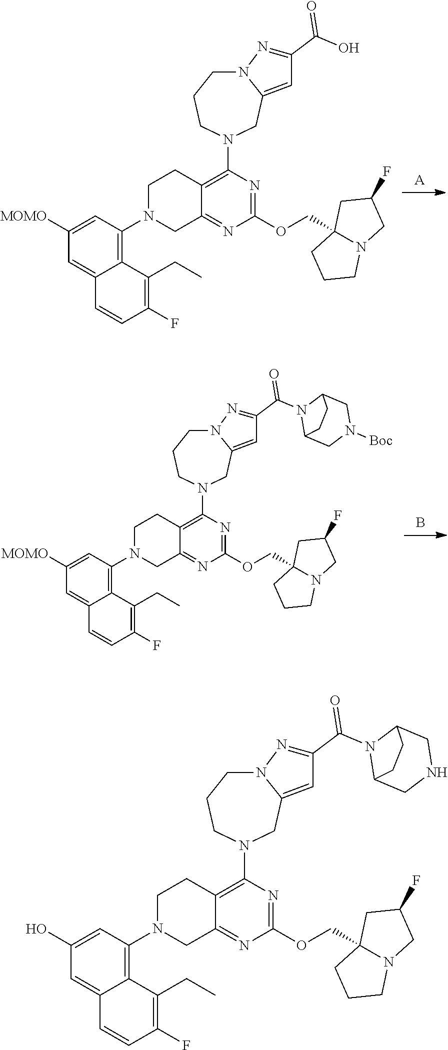

Synthesized according to Example 233 except that HCl instead of TFA was used in the last step. The title compound was obtained as yellow solid (FA salt). 1H NMR (400 MHz, methanol-d4) δ=7.55-7.48 (m, 1H), 7.19-7.10 (m, 1H), 7.00-6.93 (m, 2H), 6.77-6.69 (m, 1H), 5.53-5.28 (m, 2H), 5.04-4.90 (m, 2H), 4.64-4.47 (m, 3H), 4.36-4.16 (m, 3H), 4.13-4.00 (m, 2H), 3.72-3.63 (m, 1H), 3.62-3.46 (m, 4H), 3.45-3.35 (m, 2H), 3.22-3.03 (m, 4H), 3.01-2.92 (m, 2H), 2.80-2.70 (m, 1H), 2.56-2.33 (m, 2H), 2.33-2.09 (m, 5H), 2.09-1.86 (m, 6H), 1.16-1.05 (m, 3H); 19F NMR (400 MHz, methanol-d4) δ=−122.917, -173.733; LCMS (ESI, M+1): m/z=754.3.

### Example 259

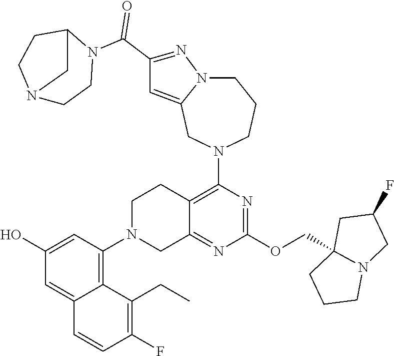 [SMILES: CCc1c(F)ccc2cc(O)cc(N3CCc4c(nc(OC[C@@]56CCCN5C[C@H](F)C6)nc4N4CCCn5nc(C(=O)N6CCN7CCC6C7)cc5C4)C3)c12]

### (1,4-diazabicyclo[3.2.1]octan-4-yl)(5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

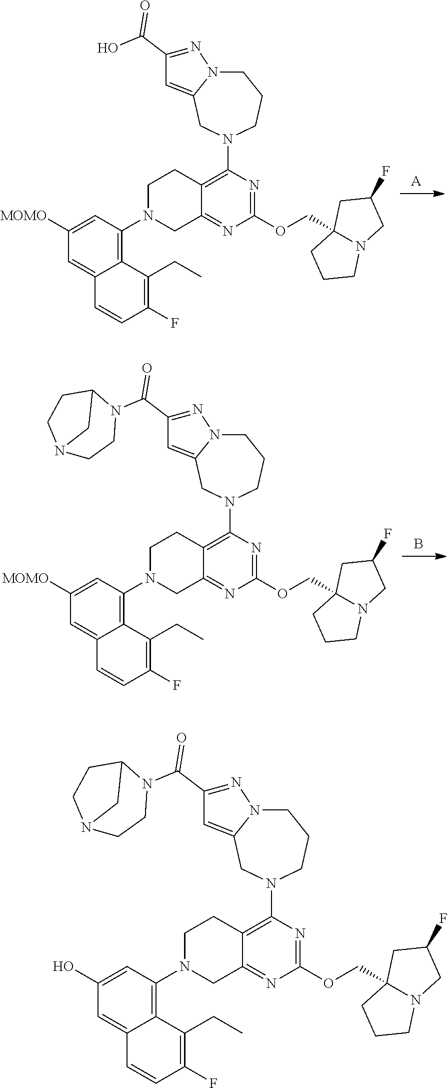

Synthesized according to Example 233 except that HCl instead of TFA was used in the last step. The title compound was obtained as brown solid (FA salt). 1H NMR (400 MHz, methanol-d4) δ=7.55-7.48 (m, 1H), 7.18-7.11 (m, 1H), 7.00-6.93 (m, 2H), 6.69-6.61 (m, 1H), 5.69-5.24 (m, 2H), 5.04-4.95 (m, 1H), 4.73-4.63 (m, 1H), 4.63-4.45 (m, 3H), 4.34-4.18 (m, 3H), 4.12-4.00 (m, 2H), 3.76-3.62 (m, 2H), 3.61-3.47 (m, 4H), 3.43-3.35 (m, 2H), 3.24-3.12 (m, 5H), 3.11-3.01 (m, 1H), 3.00-2.82 (m, 2H), 2.79-2.70 (m, 1H), 2.53-2.33 (m, 2H), 2.32-2.21 (m, 3H), 2.19-2.10 (m, 3H), 2.09-1.87 (m, 3H), 1.14-1.06 (m, 3H); 19F NMR (400 MHz, methanol-d4) δ=−122,932, -173.906; LCMS (ESI, M+1): m/z=754.5.

### Example 260

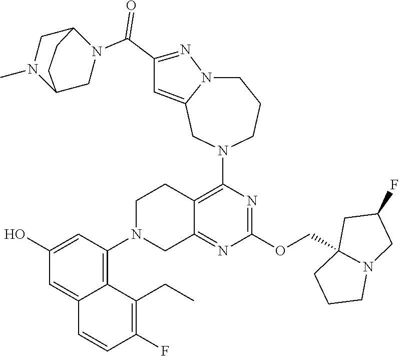 [SMILES: CCc1c(F)ccc2cc(O)cc(N3CCc4c(nc(OC[C@@]56CCCN5C[C@H](F)C6)nc4N4CCCn5nc(C(=O)N6CC7CCC6CN7C)cc5C4)C3)c12]

### (5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(5-methyl-2,5-diazabicyclo[2.2.2]octan-2-yl)methanone

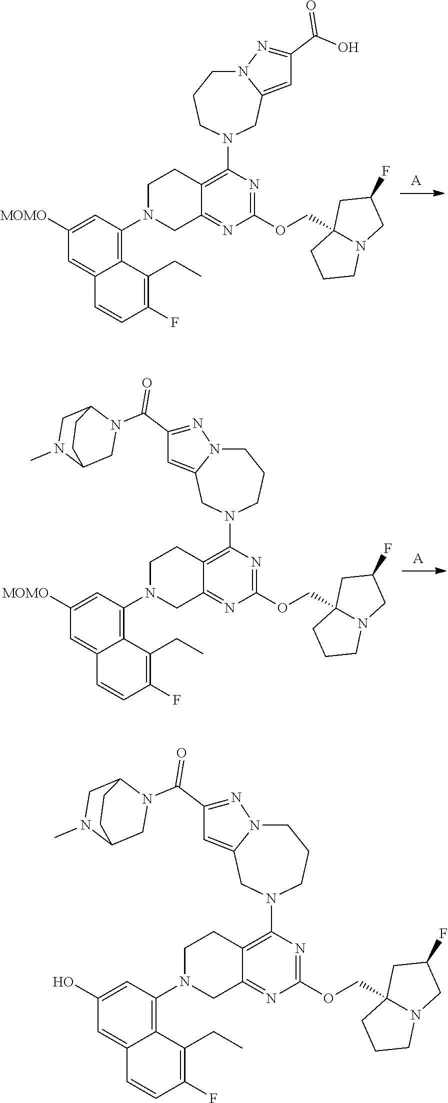

Synthesized according to Example 233 except that HCl instead of TFA was used in the last step. The title compound was obtained as yellow solid (FA salt). 1H NMR (400 MHz, methanol-d4) δ=7.55-7.48 (m, 1H), 7.15 (t, J=9.2 Hz, 1H), 6.97 (s, 2H), 6.75-6.66 (m, 1H), 5.52-5.32 (m, 1H), 5.23-4.92 (m, 2H), 4.60-4.49 (m, 2H), 4.43-4.14 (m, 4H), 4.11 (br s, 2H), 4.02-3.91 (m, 1H), 3.73-3.64 (m, 1H), 3.63-3.47 (m, 5H), 3.37 (br dd, J=5.4, 8.3 Hz, 2H), 3.25-3.08 (m, 6H), 2.79-2.70 (m, 1H), 2.63 (s, 3H), 2.54-2.34 (m, 2H), 2.34-2.20 (m, 3H), 2.19-2.13 (m, 2H), 2.10-1.98 (m, 3H), 1.92-1.75 (m, 2H), 1.16-1.06 (m, 3H); 19F NMR (400 MHz, methanol-d4) δ=−122,947, -173.906; LCMS (ESI, M+1): m/z=768.5.

### Example 261

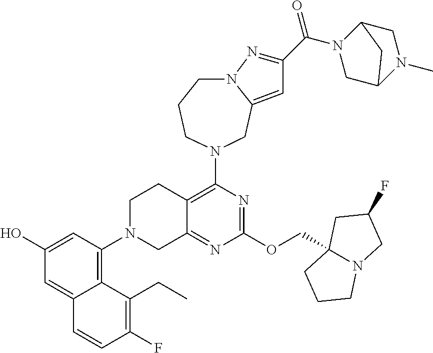 [SMILES: CCc1c(F)ccc2cc(O)cc(N3CCc4c(nc(OC[C@@]56CCCN5C[C@H](F)C6)nc4N4CCCn5nc(C(=O)N6CC7CC6CN7C)cc5C4)C3)c12]

### (5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(5-methyl-2,5-diazabicyclo[2.2.1]heptan-2-yl)methanone

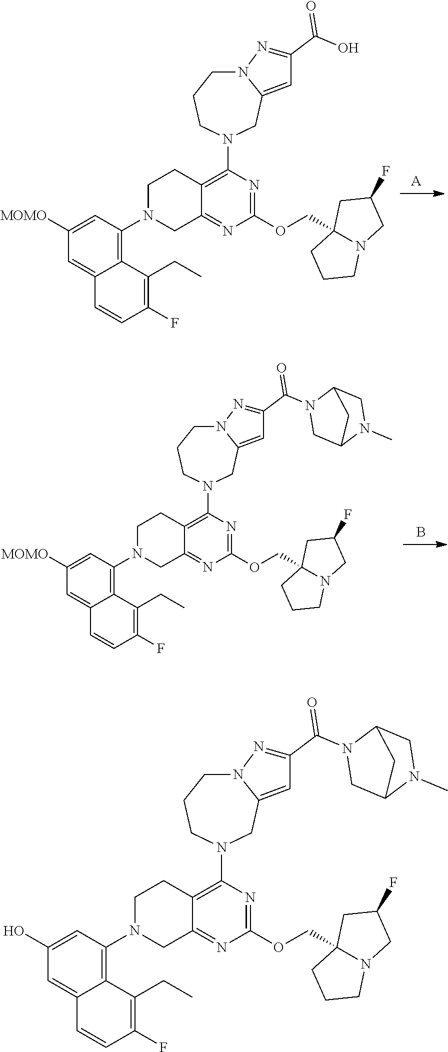

Synthesized according to Example 233 except that HCl instead of TFA was used in the last step. The title compound was obtained as yellow solid (FA salt). 1H NMR (400 MHz, dimethylsulfoxide-d6+deuterium oxide-d2) δ=7.57 (dd, J=6.0, 8.8 Hz, 1H), 7.23 (t, J=9.2 Hz, 1H), 6.98 (s, 2H), 6.69-6.54 (m, 1H), 5.49-5.38 (m, 0.5H), 5.37-5.16 (m, 1H), 5.08-4.93 (m, 1H), 4.74 (br s, 1.5H), 4.56-4.41 (m, 2H), 4.26-4.10 (m, 1H), 4.06-3.96 (m, 1H), 3.96-3.78 (m, 5H), 3.61-3.37 (m, 3H), 3.30-3.11 (m, 5H), 3.10-2.93 (m, 4H), 2.84 (br d, J=5.6 Hz, 1H), 2.69-2.59 (m, 1H), 2.54 (br d, J=3.6 Hz, 3H), 2.24-2.08 (m, 2H), 2.06-1.90 (m, 4H), 1.89-1.67 (m, 4H), 1.10-0.98 (m, 3H); 19F NMR (400 MHz, dimethylsulfoxide-d6+deuterium oxide-d2) 6=-121.070, -171.887; LCMS (ESI, M+1): m/z=754.6.

### Example 262

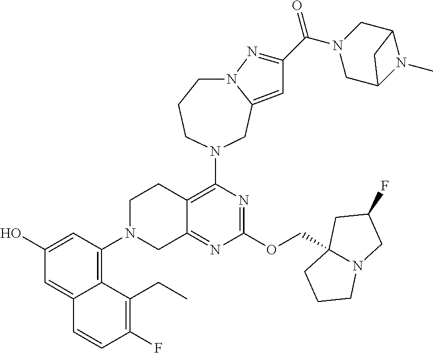 [SMILES: CCc1c(F)ccc2cc(O)cc(N3CCc4c(nc(OC[C@@]56CCCN5C[C@H](F)C6)nc4N4CCCn5nc(C(=O)N6CC7CC(C6)N7C)cc5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(6-methyl-3,6-diazabicyclo[3.1.1]heptan-3-yl)methanone

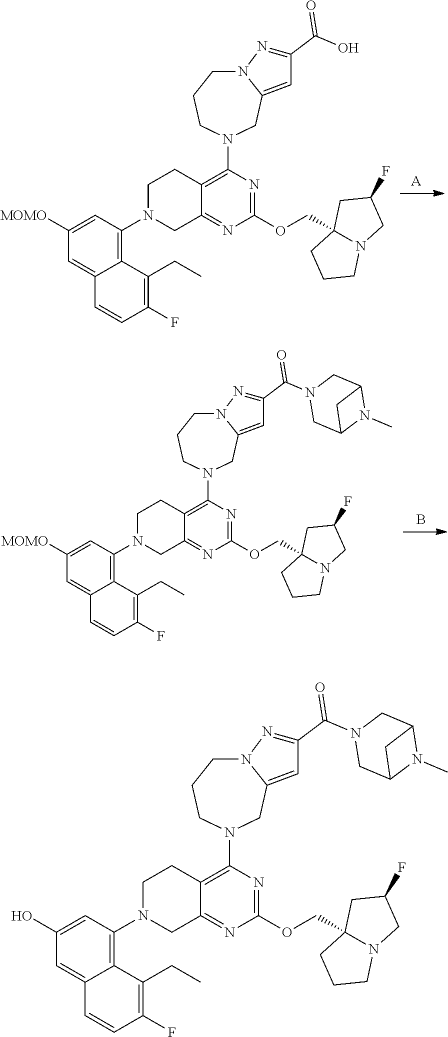

Synthesized according to Example 233 except that HCl instead of TFA was used in the last step. The title compound was obtained as brown solid (FA salt). 1H NMR (400 MHz, dimethylsulfoxide-d6+deuterium oxide-d2) 8=7.57 (dd, J=6.0, 8.8 Hz, 1H), 7.23 (t, J=9.2 Hz, 1H), 6.98 (s, 2H), 6.64 (d, J=2.0 Hz, 1H), 5.35-5.14 (m, 1H), 5.08-4.95 (m, 1H), 4.74 (br d, J=16.8 Hz, 1H), 4.57-4.42 (m, 2H), 4.26-4.03 (m, 3H), 3.83 (br d, J=16.4 Hz, 5H), 3.59 (br s, 1H), 3.55 (br s, 1H), 3.47-3.38 (m, 1H), 3.30-3.17 (m, 3H), 3.13-2.98 (m, 4H), 2.86-2.76 (m, 1H), 2.69-2.61 (m, 2H), 2.33 (br s, 1H), 2.28-2.10 (m, 3.5H), 2.06 (br d, J=0.8 Hz, 1H), 2.01-1.88 (m, 3.5H), 1.85-1.63 (m, 4H), 1.57-1.45 (m, 1H), 1.10-0.99 (m, 3H); 19F NMR (400 MHz, dimethylsulfoxide-d6+deuterium oxide-d2) δ=−121.130, -171.827; LCMS (ESI, M+1). m/z =754.3.

### Example 263

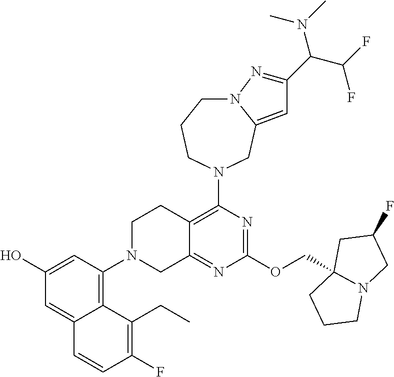 [SMILES: CCc1c(F)ccc2cc(O)cc(N3CCc4c(nc(OC[C@@]56CCCN5C[C@H](F)C6)nc4N4CCCn5nc(C(C(F)F)N(C)C)cc5C4)C3)c12]

### 4-(4-(2-(1-(dimethylamino)-2,2-difluoroethyl)-7,8-dihydro-4H-pyrazolo[1,5-a][1,4]diazepin-5(6H)-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-ol

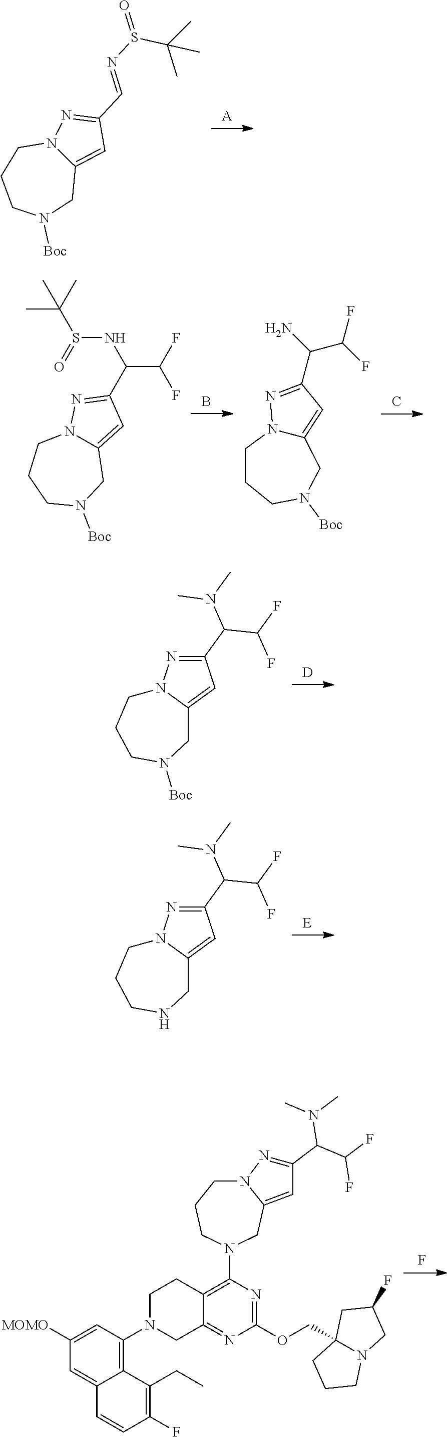

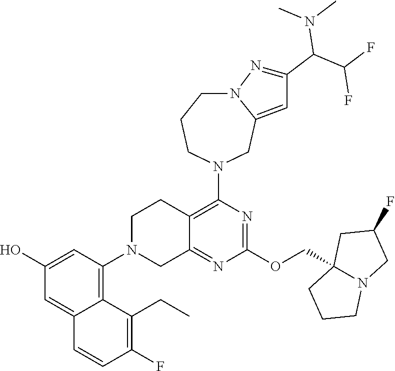 [SMILES: CCc1c(F)ccc2cc(O)cc(N3CCc4c(nc(OC[C@@]56CCCN5C[C@H](F)C6)nc4N4CCCn5nc(C(C(F)F)N(C)C)cc5C4)C3)c12]

Step A tert-butyl 2-[1-(tert-butylsulfinylamino)-2,2-difluoro-ethyl]-4,6,7,8-tetrahydropyrazolo[1,5-a][1,4]diazepine-5-carboxylate: A solution of tert-butyl 2-[(E)-tert-butylsulfinyliminomethyl]-4,6,7,8-tetrahydropyrazolo[1,5-a][1,4]diazepine-5-carboxylate (350 mg, 1.0 equiv) in THF (6.00 mL) was cooled to −78° C. t-BuOK (1 M, 2.85 mL, 3.0 equiv) and difluoromethyl(trimethyl)silane (354 mg, 3.0 equiv) were then sequentially added to the reaction and allowed to slowly warm to 0° C. for 2 hours. The reaction was diluted with water (40.0 mL) and extracted with ethyl acetate (40.0 mL). The combined organic layers were washed, dried over sodium sulphate anhydrous, filtered, concentrated, and purified with prep-TLC [SiO2, petroleum ether/ethyl actate=1/1] to afford the title compound (280 mg, 47% yield, 67% purity) as yellow oil; LCMS (ESI, M+1): m/z=421.2

Step B: tert-butyl 2-(1-amino-2,2-difluoro-ethyl)-4,6,7,8-tetrahydropyrazolo[1,5-a][1,4]diazepine-5-carboxylate: To a solution of tert-butyl 2-[1-(tert-butylsulfinylamino)-2,2-difluoro-ethyl]-4,6,7,8-tetrahydropyrazolo[1,5-a][1,4]diazepine-5-carboxylate (280 mg, 1.0 equiv) in H₂O (0.20 mL) in THF (1.0 mL) as added 12 (50.7 mg, 0.3 equiv). The mixture was stirred at 50° C. for 2 hours. The reaction was cooled to 15° C. and then quenched with 15% aqueous sodium bicarbonate (0.3 mL). The reaction was extracted with an aqueous solution of sodium thiosulfate (40.0 mL) and ethyl acetate (45.0 ml). The combined organic layers were filtered and concentrated to afford the title compound (130 mg, crude). LCMS (ESI, M+1): m/z=317.1

Step C: tert-butyl 2-[1-(dimethylamino)-2,2-difluoro-ethyl)-4,6,7,8-tetrahydropyrazolo[1,5-a]r1,4]diazepine-5-carboxylate: To a solution of tert-butyl 2-(1-amino-2,2-difluoro-ethyl)-4,6,7,8-tetrahydropyrazolo[1,5-a][1,4]diazepine-5-carboxylate (130 mg, 1.0 equiv) in dichloromethane (2.0 mL) was first added ethanoic acid (12.3 mg, 0.5 equiv), then HCHO (1.67 g, 1.53 mL, 37% purity, 50.0 equiv) and NaBH3CN (129 mg, 5.0 equiv) to the mixture. The mixture was stirred at 40° C. for 4 hours. The reaction was quenched by addition of saturated ammonium chloride aqueous solution (0.50 mL) at 25° C., and then diluted with water (20 mL) and extracted with ethyl acetate (20 mL). The combined organic layers were washed, dried over sodium sulphate anhydrous, filtered, concentrated, and purified with prep-TLC [SiO2, ethyl acetate/methanol=20/1) to afford the title compound (100 mg, 50% yield, 70% purity) as yellow oil; LCMS (ESI, M+1): m/z=345.1

Step D: 2,2-difluoro-N,N-dimethyl-1-(5,6,7,8-tetrahydro-4H-pyrazolo[1,5-a][1,4]diazepin-2-yl)ethanamine: To a solution of tert-butyl 2-[1-(dimethylamino)-2,2-difluoro-ethyl]-4,6,7,8-tetrahydropyrazolo[1,5-a][1,4]diazepine-5-carboxylate (100 mg, 1.0 equiv) in HCl/dioxane (0.50 mL) and MeCN (0.50 mL). The mixture was stirred at 25° C. for 1 hour. The reaction was filtered and concentrated under reduced pressure to afford the title compound (70 mg, crude); LCMS (ESI, M+1): m/z=245.1

The last two steps were performed according to Example 248. The title compound was obtained as white solid (FA salt)d 1H NMR (400 MHz, DMSO-d6) δ=9.86-9.59 (m, 1H), 8.24 (s, 1H), 7.59 (dd, J=6.0, 8.8 Hz, 1H), 7.24 (t, J=9.6 Hz, 1H), 7.00-6.97 (m, 2H), 6.47-6.18 (m, 1H), 6.15 (d, J=2.8 Hz, 1H), 5.31-5.15 (m, 1H), 4.95 (br dd, J=6.8, 16.0 Hz, 1H), 4.73 (br dd, J=4.0, 16.2 Hz, 1H), 4.44 (br d, J=4.0 Hz, 2H), 4.11-4.01 (m, 1H), 3.93-3.76 (m, 6H), 3.60 (br dd, J=2.4, 17.4 Hz, 1H), 3.40 (br d, J=4.4 Hz, 2H), 3.12-3.04 (m, 4H), 2.98 (br s, 1H), 2.83-2.77 (m, 1H), 2.64 (br d, J=13.6 Hz, 1H), 2.14 (s, 6H), 2.04-1.70 (m, 8H), 1.06 (t, J=7.2 Hz, 3H); LCMS (ESI, M+1): m/z=723.5

### Example 264

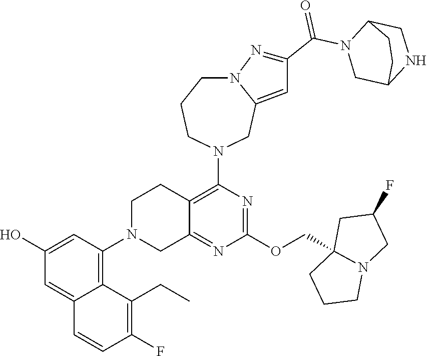 [SMILES: CCc1c(F)ccc2cc(O)cc(N3CCc4c(nc(OC[C@@]56CCCN5C[C@H](F)C6)nc4N4CCCn5nc(C(=O)N6CC7CCC6CN7)cc5C4)C3)c12]

### 2,5-diazabicyclo[2.2.2]octan-2-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

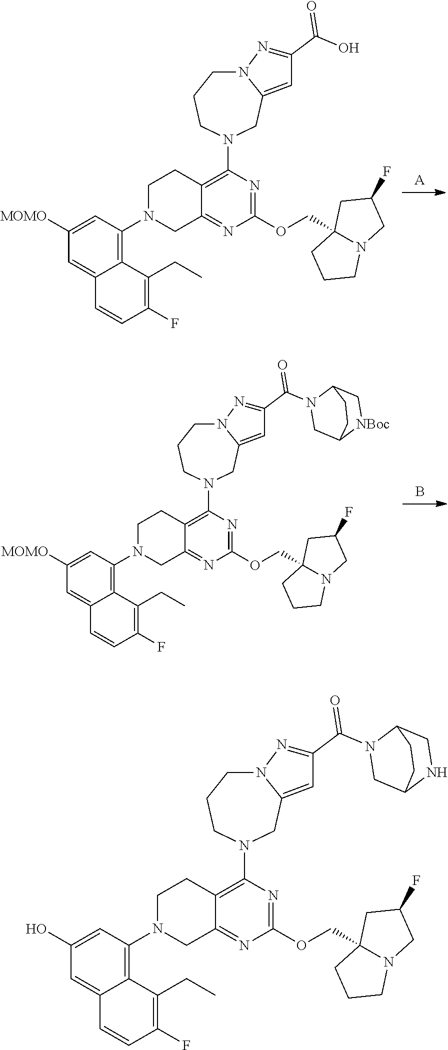

Synthesized according to Example 233 except that HCl instead of TFA was used in the last step. The title compound was obtained as off-white solid (FA salt). 1H NMR (400 MHz, methanol-d4) δ=7.55-7.49 (m, 1H), 7.14 (t, J=9.6 Hz, 1H), 6.97 (s, 2H), 6.76-6.69 (m, 1H), 5.41-5.26 (m, 1H), 5.07-4.95 (m, 1H), 4.76 (br d, J=4.4 Hz, 1H), 4.55 (br s, 2H), 4.28-3.96 (m, 6H), 3.90-3.81 (m, 1H), 3.75-3.68 (m, 2H), 3.66-3.59 (m, 1H), 3.57-3.52 (m, 1H), 3.49-3.34 (m, 6H), 3.23-3.05 (m, 3H), 2.74 (br d, J=14.4 Hz, 1H), 2.43-2.23 (m, 3H), 2.19-1.91 (m, 10H), 1.15-1.07 (m, 3H); 19F NMR (400 MHz, methanol-d4) δ=−122.962, -173.613; LCMS (ESI, M+1): m/z=754.4.

### Example 265

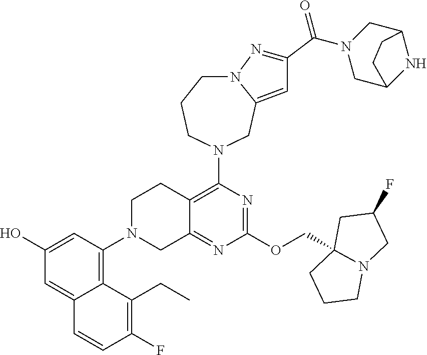 [SMILES: CCc1c(F)ccc2cc(O)cc(N3CCc4c(nc(OC[C@@]56CCCN5C[C@H](F)C6)nc4N4CCCn5nc(C(=O)N6CC7CCC(C6)N7)cc5C4)C3)c12]

### 3,8-diazabicyclo[3.2.1]octan-3-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

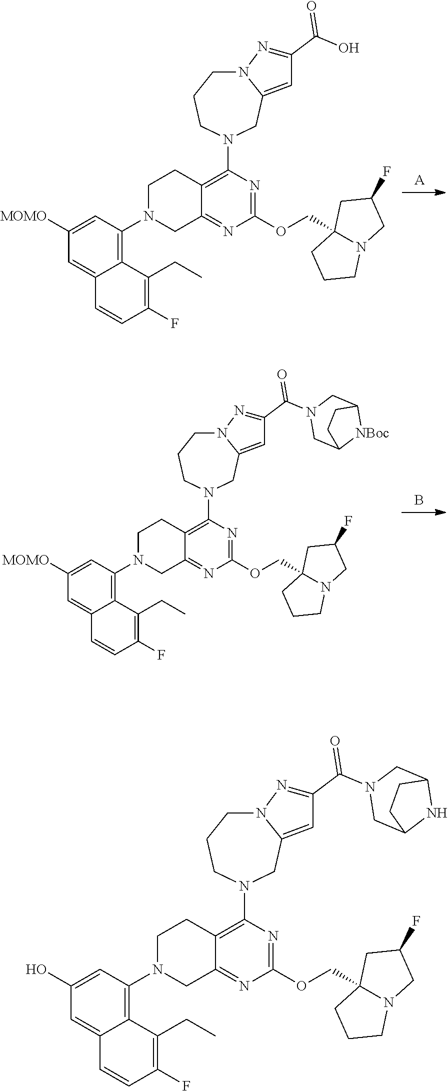

Synthesized according to Example 233 except that HCl instead of TFA was used in the last step. The title compound was obtained as off-white solid (FA salt). 1H NMR (400 MHz, methanol-d4) δ=7.57-7.47 (m, 1H), 7.14 (t, J=9.6 Hz, 1H), 6.97 (s, 2H), 6.68 (s, 1H), 5.50-5.27 (m, 1H), 5.10-4.96 (m, 2H), 4,66-4.42 (m, 4H), 4.33-4.14 (m, 3H), 4.05 (br d, J=17.6 Hz, 2H), 3.97-3.80 (m, 2H), 3.67 (br d, J=17.6 Hz, 1H), 3.57-3.37 (m, 7H), 3.22-3.10 (m, 3H), 2.75 (br d, J=14.0 Hz, 1H), 2.48-2.25 (m, 3H), 2.23-2.17 (m, 1H), 2.14-1.80 (m, 9H), 1.18-1.03 (m, 3H); 19F NMR (400 MHz, methanol-d4) δ=−123.000, -173.698; LCMS (ESI, M+1): m/z=754.2

### Example 266

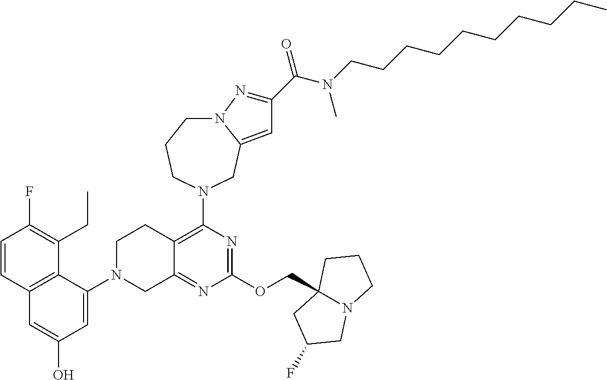 [SMILES: CCCCCCCCCCN(C)C(=O)c1cc2n(n1)CCCN(c1nc(OC[C@@]34CCCN3C[C@H](F)C4)nc3c1CCN(c1cc(O)cc4ccc(F)c(CC)c14)C3)C2]

### N-decyl-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepine-2-carboxamide

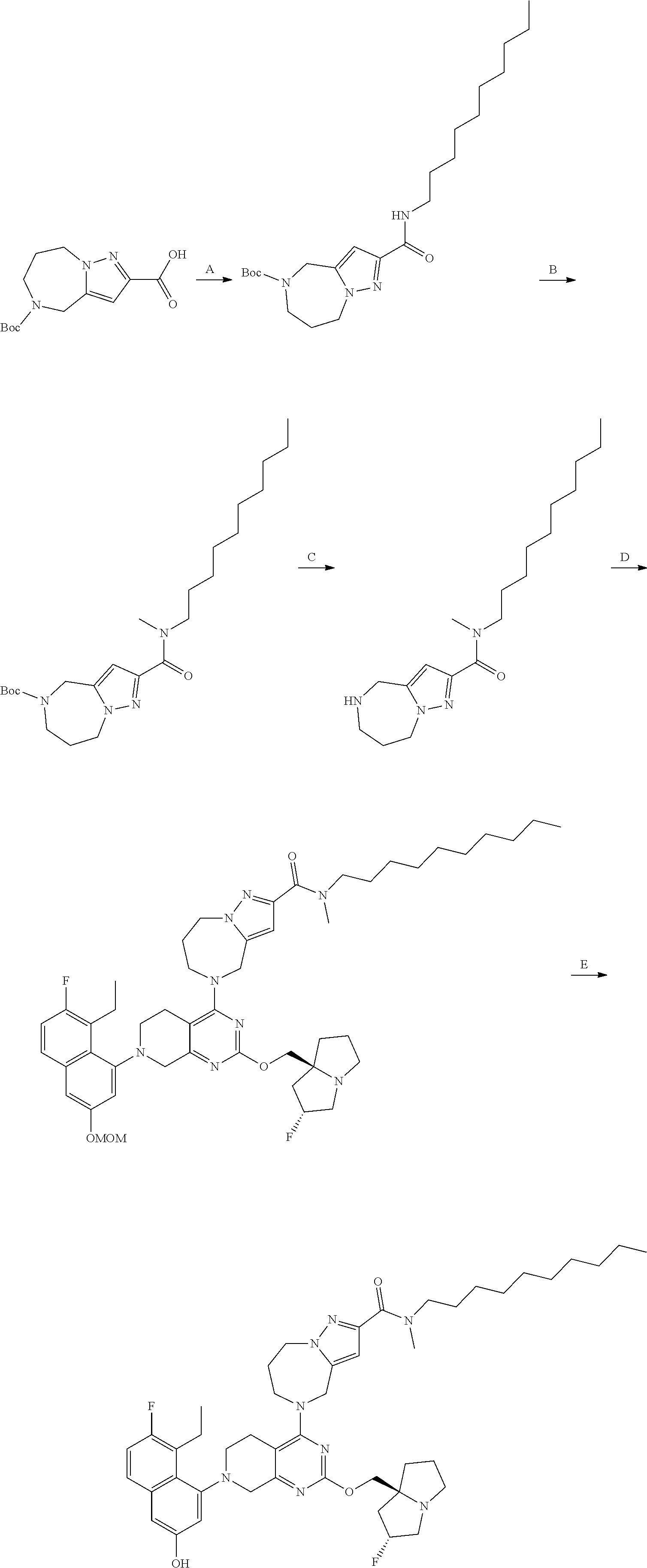

Step A. tert-butyl 2-(decylcarbamoyl)-7,8-dihydro-4H-pyrazolo[1,5-a][1,4]diazepine-5(6H)-carboxylate: To a solution of 5-(tert-butoxycarbonyl)-5,6,7,8-tetrahydro-4H-pyrazolo[1,5-a][1,4]diazepine-2-carboxylic acid (1.00 g, 1.0 equiv) and decan-1-amine (839 mg, 1.5 equiv) in DMF (6 mL) were added HATU (2.70 g, 2.0 equiv) and N,N-diethylpropan-2-amine (1.38 g, 3.0 equiv). The mixture was stirred at 20° C. for 18 hours. The mixture was diluted with water (10 mL) and extracted with ethyl acetate (2×10 mL). The organic layer was washed with brine (10 mL) and dried over Na₂SO₄. The solvent was concentrated and purified with reversed phase flash chromatography [C18, 0.1% formic acid condition] to afford the title compound (1.40 g, 94% yield) as yellow solid; LCMS (ESI, M+1): m/z=421.3.

Step B. tert-butyl 2-(decyl(methyl)carbamoyl)-7,8-dihydro-4H-pyrazolo[1,5-a][1,4]diazepine-5(6H)-carboxylate: To a solution of tert-butyl 2-(decyl(methyl)carbamoyl)-7,8-dihydro-4H-pyrazolo[1,5-a][1,4]diazepine-5(6H)-carboxylate (643 mg, 1.0 equiv) in THF (6 mL) was added NaH (122 mg, 60% purity, 2.0 equiv) slowly at 0° C. The mixture was stirred at 25° C. for 20 minutes. To the mixture was added CH₃I (2.39 g, 11 equiv) slowly at 0° C. The mixture was stirred at 25° C. for 12 hours. The mixture was quenched by saturated NH₄Cl aqueous (10 mL) at 0° C. and extracted with ethyl acetate (2×20 mL). The organic layer was washed with brine (20 mL) and dried over Na₂SO₄. The solvent was concentrated and purified with reversed phase flash chromatography [C18, 0.1% formic acid condition] to afford the title compound (500 mg, 74% yield) as yellow solid; LCMS (ESI, M+1): m/z=435.4.

Step C. N-decyl-N-methyl-5,6,7,8-tetrahydro-4H-pyrazolo[1,5-a][1,4]diazepine-2-carboxamide: To a solution of tert-butyl 2-(decyl(methyl)carbamoyl)-7,8-dihydro-4H-pyrazolo[1,5-a][1,4]diazepine-5(6H)-carboxylate (500 mg, 1.0 equiv) in MeOH (2.5 mL) was added HCl-MeOH (4 M, 5 mL, 17 equiv) at 0° C. The mixture was stirred at 0° C. for 0.5 hours. The mixture was concentrated, and the residue was dissolved in MeOH (5 mL). NaHCO₃ (200 mg) was added, and the mixture was stirred at 25° C. for 0.5 hours. The mixture was filtered and concentrated to afford the title compound (300 mg, crude) as yellow solid.

The last two steps were performed according to Example 248 (step B and C). The title compound was obtained as pink solid (FA salt). 1H NMR (400 MHz, METHANOL-d4) δ=7.50 (dd, J=6.0, 8.8 Hz, 1H), 7.13 (t, J=9.2 Hz, 1H), 6.96 (s, 2H), 6.61-6.56 (m, 1H), 5.40-5.20 (m, 1H), 5.07-4.92 (m, 1H), 4.85-4.76 (m, 1H), 4.60-4.45 (m, 2H), 4.27-3.95 (m, 5H), 3.75-3.60 (m, 2H), 3.58-3.44 (m, 2H), 3.37 (dt, J=2.4, 6.8 Hz, 3H), 3.30-3.12 (m, 5H), 3.11-2.98 (m, 3H), 2.72 (br d, J=13.6 Hz, 1H), 2.39-2.17 (m, 3H), 2.17-1.95 (m, 4H), 1.90 (br dd, J=6.0, 13.6 Hz, 1H), 1.63 (br s, 2H), 1.40-1.17 (m, 14H), 1.15-1.05 (m, 3H), 0.91-0.83 (m, 3H); LCMS (ESI, M+1): m/z=813.5.

### Example 267

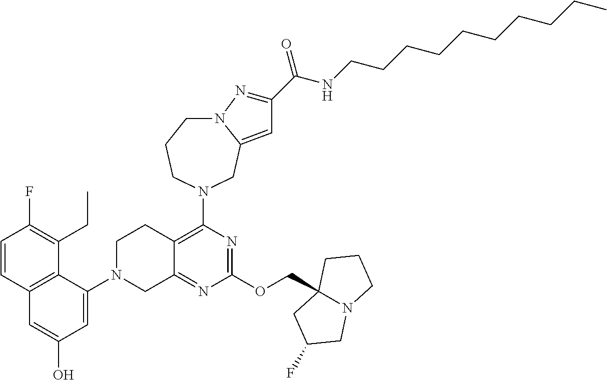 [SMILES: CCCCCCCCCCNC(=O)c1cc2n(n1)CCCN(c1nc(OC[C@@]34CCCN3C[C@H](F)C4)nc3c1CCN(c1cc(O)cc4ccc(F)c(CC)c14)C3)C2]

### N-decyl-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepine-2-carboxamide

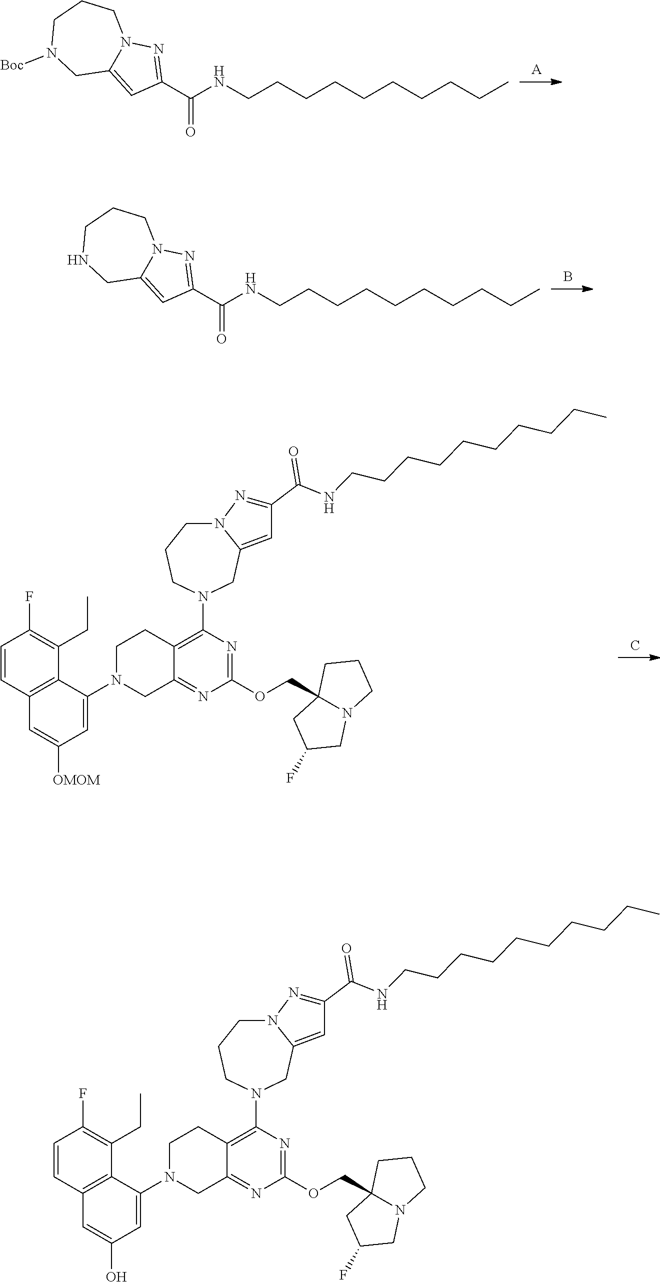

Step A. N-decyl-5,6,7,8-tetrahydro-4H-pyrazolo[1,5-a][1,4]diazepine-2-carboxamide: To a solution of tert-butyl 2-(decylcarbamoyl)-7,8-dihydro-4H-pyrazolo[1,5-a][1,4]diazepine-5(6H)-carboxylate (321 mg, 1.0 equiv) in MeOH (2 mL) was added HCl•MeOH (4 M, 4 mL, 21 equiv) at 0° C. The mixture was stirred at 0° C. for 0.5 hours. The mixture was concentrated to afford the title compound (200 mg, crude) as yellow oil; LCMS (ESI, M+1): m/z =321.4.

The last two steps were performed according to Example 248 (step A and B). The title compound was obtained as pink solid. 1H NMR (400 MHz, METHANOL-d4) δ=7.51 (dd, J=5.6, 9.2 Hz, 1H), 7.14 (t, J=9.2 Hz, 1H), 7.02-6.95 (m, 2H), 6.69 (s, 1H), 5.44-5.22 (m, 1H), 5.05-4.92 (m, 3H), 4.63-4.45 (m, 2H), 4.27-4.10 (m, 3H), 4.09-3.96 (m, 2H), 3.65 (br d, J=17.6 Hz, 1H), 3.54 (br d, J=9.4 Hz, 1H), 3.42-3.32 (m, 6H), 3.28-3.21 (m, 1H), 3.18 (br dd, J=2.3, 10.8 Hz, 1H), 3.15-3.06 (m, 1H), 2.73 (br d, J=14.8 Hz, 1H), 2.45-2.21 (m, 3H), 2.16 (br d, J=9.2 Hz, 1H), 2.12-1.99 (m, 3H), 1.98-1.85 (m, 1H), 1.58 (br t, J=7.2 Hz, 2H), 1.43-1.22 (m, 14H), 1.15-1.06 (m, 3H), 0.92-0.89 (m, 1H), 0.94-0.82 (m, 2H); LCMS (ESI, M+1): m/z=799.5.

### Example 268

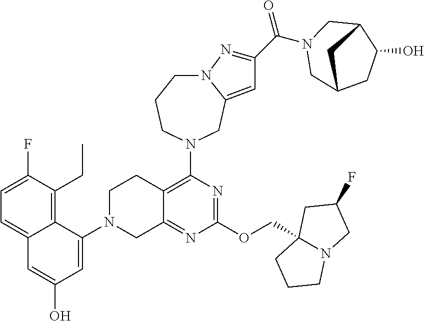 [SMILES: CCc1c(F)ccc2cc(O)cc(N3CCc4c(nc(OC[C@@]56CCCN5C[C@H](F)C6)nc4N4CCCn5nc(C(=O)N6C[C@@H]7C[C@H](C6)[C@H](O)C7)cc5C4)C3)c12]

### (5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)((1R,5R,6R)-6-hydroxy-3-azabicyclo[3.2.1]octan-3-yl)methanone

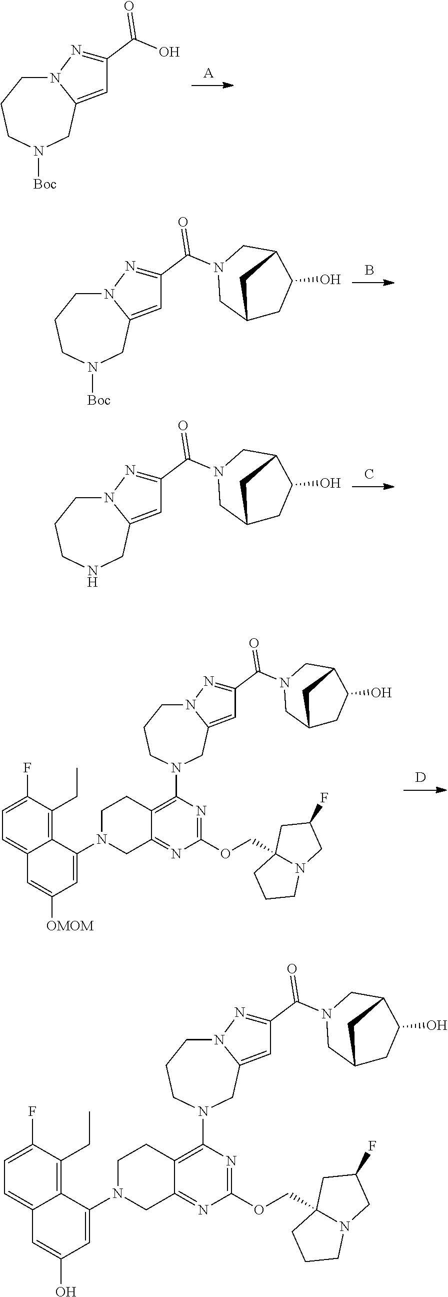

Step A. tert-butyl 2-((1R,5R,6R)-6-hydroxy-3-azabicyclo[3.2.1]octane-3-carbonyl)-7,8-dihydro-4H-pyrazolo[1,5-a][1,4]diazepine-5(6H)-carboxylate: To a mixture of 5-(tert-butoxycarbonyl)-5,6,7,8-tetrahydro-4H-pyrazolo[1,5-a][1,4]diazepine-2-carboxylic acid (5.00 g, 1.0 equiv) in DMF (100 mL) were added N,N-diethylpropan-2-amine (6.89 g, 3.0 equiv), HATU (13.5 g, 2.0 equiv) and (1R,5R,6R)-3-azabicyclo[3.2.1]octan-6-ol (4.52 g, 2.0 equiv). The mixture was stirred at 20° C. for 12 hours. The residue was filtered and washed with DMF (1 mL) and purified by reversed phase flash chromatography [C18, 0.1% formic acid condition] to afford the title compound (5.00 g, 71% yield) as white solid; LCMS (ESI, M+1): m/z=391.2.

Step B. ((1R,5R,6R)-6-hydroxy-3-azabicyclo[3.2.1]octan-3-yl)(5,6,7,8-tetrahydro-4H-pyrazolo[1,5-a][1,4]diazepin-2-yl)methanone: To a solution of tert-butyl 2-((1R,5R,6R)-6-hydroxy-3-azabicyclo[3.2.1]octane-3-carbonyl)-7,8-dihydro-4H-pyrazolo[1,5-a][1,4]diazepine-5(6H)-carboxylate (5.00 g, 1.0 equiv) in MeCN (10 mL) was added HCl•dioxane (4 M, 20 mL, 6.2 equiv) at 0° C. The mixture was stirred at 0° C. for 0.5 hours. The reaction was concentrated. The residue was adjusted to pH 8 with 20% NaOH aqueous solution (5 mL) and extracted with EtOAc (3×10 mL). The combined organic layers were dried over anhydrous sodium sulfate and concentrated to afford the title compound (3.50 g, 94% yield) as white solid.

The last two steps were performed according to Example 248. The title compound was obtained as pink solid. 1H NMR (400 MHz, METHANOL-d4) δ=7.51 (dd, J=6.0, 9.2 Hz, 1H), 7.14 (t, J=9.2 Hz, 1H), 6.97 (s, 2H), 6.69-6.47 (m, 1H), 5.51-5.26 (m, 1H), 5,00-4.91 (m, 1H), 4.79-4.63 (m, 1H), 4.60-4.33 (m, 4H), 4.30-4.19 (m, 3H), 4.18-4.03 (m, 3H), 3.68 (br d, J=17.6 Hz, 1H), 3.60-3.44 (m, 4H), 3.41-3.34 (m, 2H), 3.29-3.11 (m, 4H), 3.02-2.85 (m, 1H), 2.73 (br d, J=13.2 Hz, 1H), 2.50-2.15 (m, 7H), 2.15-1.88 (m, 5H), 1.78-1.65 (m, 2H), 1.17-1.01 (m, 3H); LCMS (ESI, M+1). m/z=769.5.

### Example 269

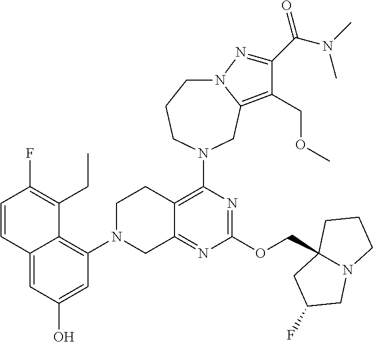 [SMILES: CCc1c(F)ccc2cc(O)cc(N3CCc4c(nc(OC[C@@]56CCCN5C[C@H](F)C6)nc4N4CCCn5nc(C(=O)N(C)C)c(COC)c5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-3-(methoxymethyl)-N,N-dimethyl-5, 6,7, 8-tetrahydro-4H-pyrazolo[1,5-a][1,4]diazepine-2-carboxamide

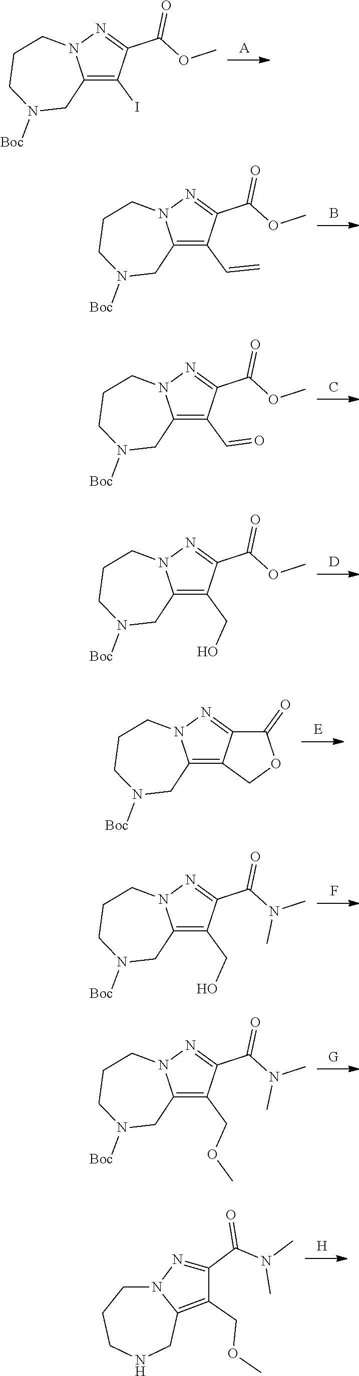

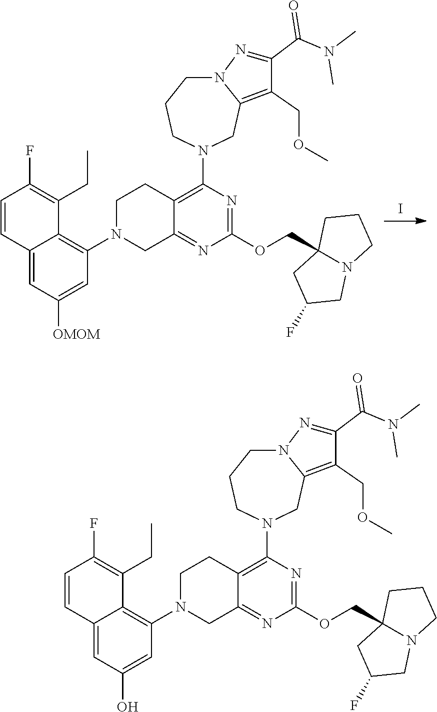

Step A: 5-tert-butyl 2-methyl 3-vinyl-7,8-dihydro-4H-pyrazolo[1,5-a][1,4]diazepine-2,5(6H)-dicarboxylate: A mixture of 5-tert-butyl 2-methyl 3-iodo-7,8-dihydro-4H-pyrazolo[1,5-a][1,4]diazepine-2,5(6H)-dicarboxylate (2.80 g,, 1.0 equiv), 4,4,5,5-tetramethyl-2-vinyl-1,3,2-dioxaborolane (2.05 g, 2.0 equiv), CataCXium A Pd G3 (968 mg, 0.2 equiv) and NaHCO₃ (1.68 g, 3.0 equiv) in dioxane (30 mL) and H₂O (6 mL) was degassed and purged with N2 3 times. Then the mixture was stirred at 80° C. for 3 hours under N2 atmosphere. The mixture was diluted with H₂O (30 mL) and extracted with ethyl acetate (3×30 mL). The combined organic layers were dried over anhydrous Na₂SO₄, concentrated and purified by reversed phase flash chromatography [C18, 0.1% formic acid condition] to afford the title compound (2.1 g, 92% yield) as yellow solid; LCMS (ESI, M+1): m/z=322.1.

Step B: 5-tert-butyl 2-methyl 3-formyl-7,8-dihydro-4H-pyrazolo[1,5-a][1,4]diazepine-2,5(6H)-dicarboxylate: To a solution of 5-tert-butyl 2-methyl 3-vinyl-7,8-dihydro-4H-pyrazolo[1,5-a][1,4]diazepine-2,5(6H)-dicarboxylate (2.1 g, 1.0 equiv) in THF (25 mL) and H₂O (25 mL) were added K20sO4·2H₂O (120 mg, 0.05 equiv) and NaIO4 (2.80 g, 2.0 equiv) slowly at 0-5° C. The mixture was stirred at 20° C. for 3 hours. The mixture was diluted with H₂O (30 mL) and extracted with ethyl acetate (3×30 mL). The combined organic layers were dried over anhydrous Na₂SO₄, concentrated, and purified by reversed phase flash chromatography [C18, 0.1% formic acid condition] to afford the title compound (1.8 g, 72% yield) as yellow solid. LCMS (ESI, M+1): m/z=324.2.

Step C: 5-tert-butyl 2-methyl 3-(hydroxymethyl)-7,8-dihydro-4H-pyrazolo[1,5-a][1,4]diazepine-2,5(6H)-dicarboxylate: To a solution of 5-tert-butyl 2-methyl 3-formyl-7,8-dihydro-4H-pyrazolo[1,5-a][1,4]diazepine-2,5(6H)-dicarboxylate (1.5 g, 1.0 equiv) in EtOH (20 mL) was added NaBH4 (180 mg, 1.0 equiv) slowly under N2 atmosphere. The reaction was stirred at 0° C. for 2 hours. The solution was diluted with H₂O (30 mL) and extracted with ethyl acetate (3×30 mL). The combined organic layers were dried over anhydrous Na₂SO₄, concentrated, and purified by reversed phase flash chromatography [C18, 0.1% formic acid condition] to afford the title compound (1.5 g, 99% yield) as yellow oil.

Step D: tert-butyl 3-oxo-6,7,8,10-tetrahydro-1H-furo[3′,4′:3,4]pyrazolo[1,5-a][1,4]diazepine-9(3H)-carboxylate: To a solution of 5-tert-butyl 2-methyl 3-(hydroxymethyl)-7,8-dihydro-4H-pyrazolo[1,5-a][1,4]diazepine-2,5(6H)-dicarboxylate (1.5 g, 1.0 equiv) in THF (90 mL) was added t-BuONa (691 mg, 2.0 equiv) at 0° C. The mixture was stirred at 20° C. for 2 hours. The mixture was diluted with H₂O (100 mL) and extracted with ethyl acetate (4×50 mL). The combined organic layers were dried over Na₂SO₄, concentrated, and purified by reversed phase flash chromatography [C18, 0.1% formic acid condition] to afford the title compound (0.8 g, 75% yield) as yellow oil. LCMS (ESI, M+1): m/z=294.1.

Step E: tert-butyl 2-(dimethylcarbamoyl)-3-(hydroxymethyl)-7,8-dihydro-4H-pyrazolo[1,5-a][1,4]diazepine-5(6H)-carboxylate: To a solution of tert-butyl 3-oxo-6,7,8,10-tetrahydro-1H-furo[3′,4′:3,4]pyrazolo[1,5-a][1,4]diazepine-9(3H)-carboxylate (0.45 g, 1.0 equiv) and N-methylmethanamine (2 M, 1.53 mL, 2.0 equiv) in DMF (4.5 mL) were added HATU (1.17 g, 2.0 equiv) and N,N-diethylpropan-2-amine (595 mg, 3.0 equiv). The mixture was stirred at 20° C. for 12 hours. The mixture was purified by reversed phase flash chromatography [C18, 0.1% formic acid condition] to afford the title compound (0.23 g, 42% yield) as yellow oil.

Step F: tert-butyl 2-(dimethylcarbamoyl)-3-(methoxymethyl)-7,8-dihydro-4H-pyrazolo[1,5-a][1,4]diazepine-5(6H)-carboxylate: To a solution of tert-butyl 2-(dimethylcarbamoyl)-3-(hydroxymethyl)-7,8-dihydro-4H-pyrazolo[1,5-a][1,4]diazepine-5(6H)-carboxylate (180 mg, 1.0 equiv) in THE (2 mL) was added NaH (31.9 mg, 60% purity, 1.5 equiv) at 0-5° C. under N2. The mixture was stirred at 0° C. for 0.5 hours, then Mel (151 mg, 2.0 equiv) was added. The mixture was stirred at 20° C. for 1 hour. The mixture was quenched by ice water (20 mL) and extracted with ethyl acetate (3×20 mL). The combined organic layers were dried over Na₂SO₄, concentrated, and purified by reversed phase flash chromatography [C18, 0.1% NH₃•H₂O condition] to afford the title compound (165 mg, 88% yield) as white solid.

Step G: 3-(methoxymethyl)-N,N-dimethyl-5,6,7,8-tetrahydro-4H-pyrazolo[1,5-a][1,4]diazepine-2-carboxamide: To a solution of tert-butyl 2-(dimethylcarbamoyl)-3-(methoxymethyl)-7,8-dihydro-4H-pyrazolo[1,5-a][1,4]diazepine-5(6H)-carboxylate (100 mg, 1.0 equiv) in DCM (0.5 mL) was added HCl•dioxane (4 M, 2.0 mL, 17.1 equiv) in ACN (2 mL) at 0° C. The mixture was stirred at 20° C. for 1 hour. The solution was concentrated to afford the title compound (120 mg, HCl salt, crude) as yellow solid.

The last two steps were performed according to Example 248. The title compound was obtained as yellow solid (FA salt). 1H NMR (400 MHz, METHANOL-d4) δ=7.51 (dd, J=6.0, 8.8 Hz, 1H), 7.14 (t, J=9.2 Hz, 1H), 7.00-6.92 (m, 2H), 5.47-5.24 (m, 1H), 5.03-4.95 (m, 2H), 4.86-4.79 (m, 1H), 4.57-4.38 (m, 4H), 4.21-3.98 (m, 5H), 3.74-3.65 (m, 1H), 3.52-3.45 (m, 1H), 3.45-3.36 (m, 4H), 3.35-3.32 (m, 1H), 3.26-3.22 (m, 3H), 3.21-3.13 (m, 3H), 3.13-3.06 (m, 6H), 2.74-2.60 (m, 1H), 2.43-2.22 (m, 3H), 2.21-2.11 (m, 2H), 2.10-2.00 (m, 2H), 1.99-1.86 (m, 1H), 1.11 (t, J=7.2 Hz, 3H); LCMS (ESI, M+1): m/z=731.4.

### Example 270

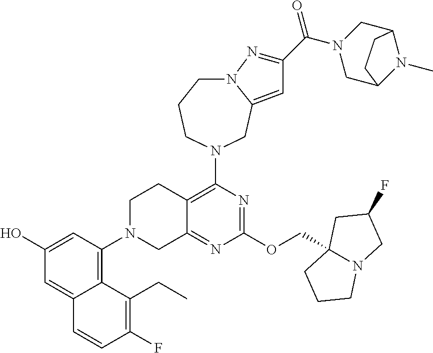 [SMILES: CCc1c(F)ccc2cc(O)cc(N3CCc4c(nc(OC[C@@]56CCCN5C[C@H](F)C6)nc4N4CCCn5nc(C(=O)N6CC7CCC(C6)N7C)cc5C4)C3)c12]

### (5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(8-methyl-3,8-diazabicyclo[3.2.1]octan-3

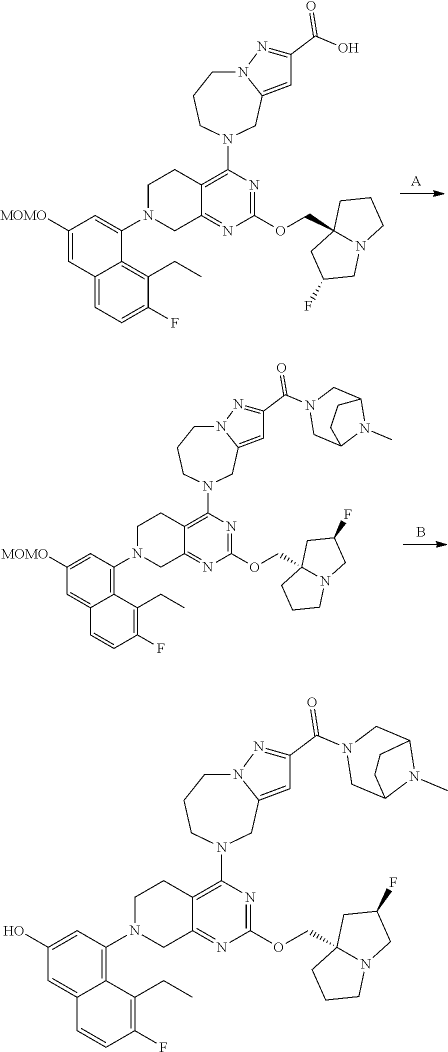

Synthesized according to Example 233 except that HCl instead of TFA was used in the last step. The title compound was obtained as yellow solid (FA salt). 1H NMR (400 MHz, dimethylsulfoxide-d6+deuterium oxide-d2) δ=7.55 (dd, J=6.0, 9.2 Hz, 1H), 7.21 (t, J=9.2 Hz, 1H), 6.97 (br d, J=2.4 Hz, 2H), 6.56 (s, 1H), 5.46-5.22 (m, 1H), 4.98 (br t, J=18.8 Hz, 1H), 4.81-4.67 (m, 1H), 4.55-4.39 (m, 3H), 4.28-4.12 (m, 2H), 4.11-3.97 (m, 2H), 3.81 (br d, J=16.8 Hz, 2H), 3.60-3.49 (m, 2H), 3.44 (br s, 3H), 3.36-3.12 (m, 6H), 3.09-2.91 (m, 3H), 2.65 (br d, J=14.8 Hz, 1H), 2.40 (s, 3H), 2.26-2.10 (m, 3H), 2.00 (br s, 3H), 1.96-1.72 (m, 4H), 1.67-1.49 (m, 2H), 1.01 (br d, J=3.6 Hz, 3H); 19F NMR (400 MHz, dimethylsulfoxide-d6+deuterium oxide-d2) 6 =−123.905, -171.962; LCMS (ESI, M+1): m/z=768.5.

### Example 271

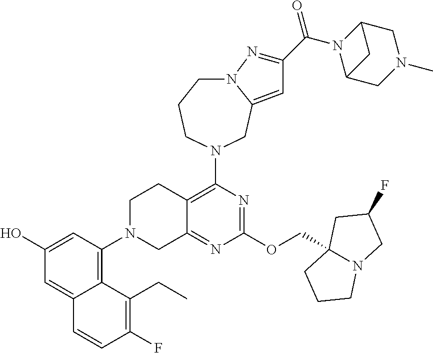 [SMILES: CCc1c(F)ccc2cc(O)cc(N3CCc4c(nc(OC[C@@]56CCCN5C[C@H](F)C6)nc4N4CCCn5nc(C(=O)N6C7CC6CN(C)C7)cc5C4)C3)c12]

### (5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(3-methyl-3,6-diazabicyclo[3.1.1]heptan-6-yl)methanone

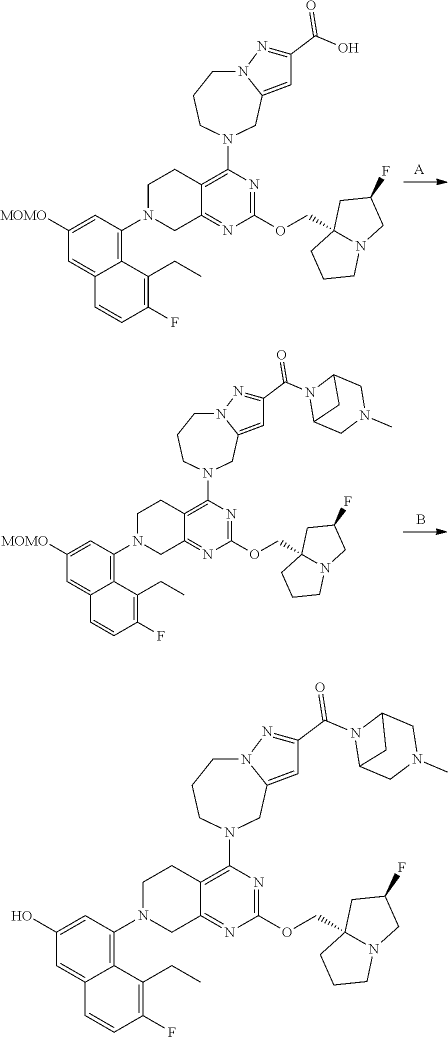

Synthesized according to Example 233 except that HCl instead of TFA was used in the last step. The title compound was obtained as yellow solid (FA salt). 1H NMR (400 MHz, dimethylsulfoxide-d6+deuterium oxide-d2) δ=7.55 (dd, J=6.0, 9.2 Hz, 1H), 7.21 (t, J=9.2 Hz, 1H), 7.02-6.91 (m, 2H), 6.63 (dd, J=3.6, 6.4 Hz, 1H), 5.51-5.30 (m, 1H), 5.05-4.82 (m, 2H), 4.81-4.65 (m, 1H), 4.49-4.30 (m, 3H), 4.26-4.12 (m, 3H), 3.80 (br d, J=16.8 Hz, 2H), 3.62-3.35 (m, 5H), 3.26-2.98 (m, 7H), 2.93-2.72 (m, 2H), 2.70-2.62 (m, 1H), 2.47-2.41 (m, 1H), 2.37-2.31 (m, 1H), 2.26 (s, 4H), 2.18-2.08 (m, 2H), 2.07-1.95 (m, 2H), 1.93-1.81 (m, 3H), 1.00 (q, J=6.8 Hz, 3H); 19F NMR (400 MHz, dimethylsulfoxide-d6+deuterium oxide-d2) δ=−120.659, -172.172; LCMS (ESI, M+1): m/z=754.4.

### Example 272

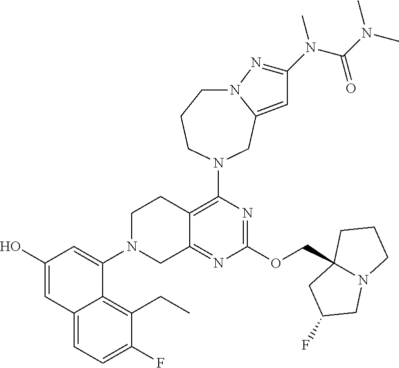 [SMILES: CCc1c(F)ccc2cc(O)cc(N3CCc4c(nc(OC[C@@]56CCCN5C[C@H](F)C6)nc4N4CCCn5nc(N(C)C(=O)N(C)C)cc5C4)C3)c12]

### 1-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-1,3,3-trimethylurea

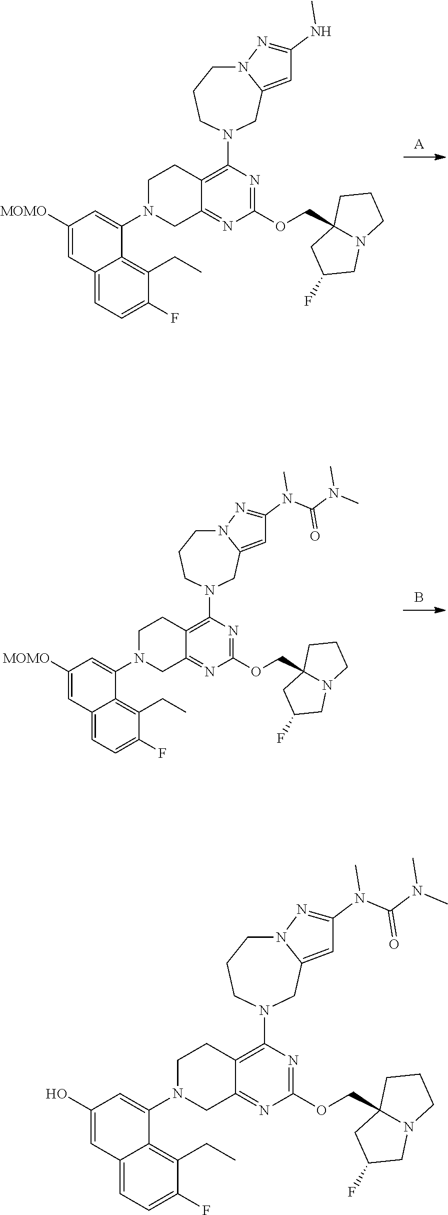

Step A. 1-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-1,3,3-trimethylurea: To a mixture of 5-(7-(8-ethyl-7-fluoro-3-(methoxymethoxy)naphthalen-1-yl)-2-(((2R,7aS)-2-fluorotetrahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepin-2-amine (100 mg, 1.0 equiv) and dimethylcarbamic chloride (39.0 mg, 2.5 equiv) in THE (1 mL) was added pyridine (4.90 g, 427 equiv). The reaction was stirred at 20° C. for 2 hours. The mixture was concentrated and purified by reversed phase chromatography [C18, 0.1% formic acid condition] to afford the title compound (97.0 mg, 87% yield) as purple solid; LCMS (ESI, M+1): m/z=760.2.

Step B. 1-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-5,6,7,8-tetrahydro-4H-pyrazolo[1,5-a][1,4]diazepin-2-yl)-1,3,3-trimethylurea: To a mixture of 1-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-1,3,3-trimethylurea (87.0 mg, 1.0 equiv) in ACN (215 μL) was added HCl•MeOH (4 M, 429 μL, 15 equiv). The reaction was stirred at 20° C. for 1 hour. The mixture was concentrated, dissolved in methanol (1 mL), neutralized with solid NaHCO₃, filtered, and purified by prep-HPLC [Phenomenex luna C18 150×25 mm×10 m; A: water (FA), B: ACN; B %: 20%-50% over 10 min] and lyophilized to afford the title compound (20.8 mg, 23% yield, 0.48 equiv FA) as yellow solid. 1H NMR (400 MHz, methanol-d4) δ=7.55-7.48 (m, 1H), 7.19-7.11 (m, 1H), 7.00-6.95 (m, 2H), 5.99-5.95 (m, 1H), 5.46-5.27 (m, 1H), 4.97 (br d, J=16.8 Hz, 1H), 4.80-4.73 (m, 1H), 4.43-4.37 (m, 2H), 4.29-4.14 (m, 3H), 4.09-4.02 (m, 1H), 3.99-3.90 (m, 1H), 3.73-3.65 (m, 1H), 3.56-3.51 (m, 1H), 3.51-3.43 (m, 2H), 3.43-3.39 (m, 2H), 3.24-3.17 (m, 2H), 3.14 (s, 4H), 2.77-2.73 (m, 6H), 2.73-2.68 (m, 1H), 2.34 (br s, 1H), 2.32-2.23 (m, 2H), 2.16 (br d, J=10.8 Hz, 5H), 1.99-1.90 (m, 1H), 1.10 (dt, J=1.6, 7.2 Hz, 3H); 19F NMR (400 MHz, methanol-d4) δ=−122.925, -173.818; LCMS (ESI, M+1): m/z=716.5.

### Example 273

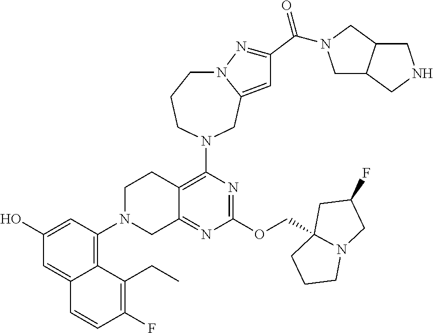 [SMILES: CCc1c(F)ccc2cc(O)cc(N3CCc4c(nc(OC[C@@]56CCCN5C[C@H](F)C6)nc4N4CCCn5nc(C(=O)N6CC7CNCC7C6)cc5C4)C3)c12]

### (5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(hexahydropyrrolo[3,4-c]pyrrol-2(1H)-yl)methanone

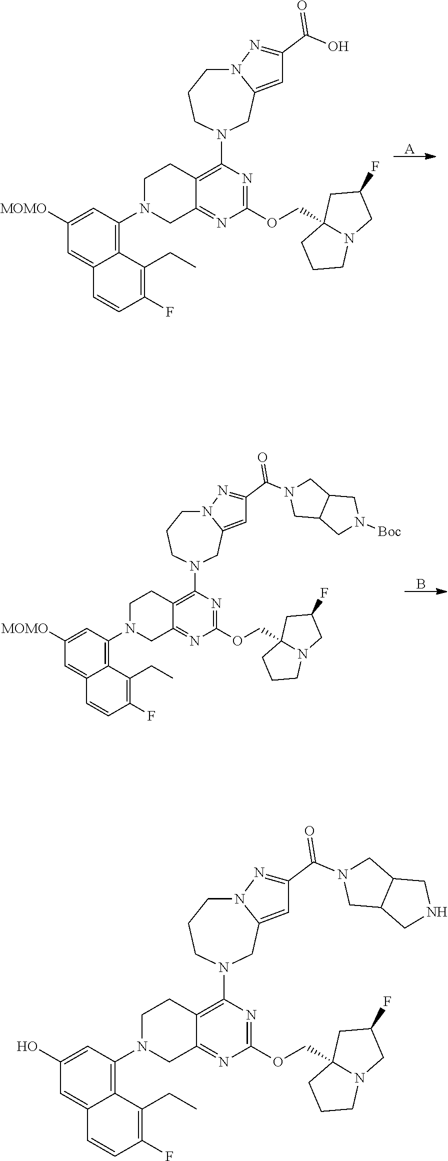

Synthesized according to Example 233 except that HCl instead of TFA was used in the last step. The title compound was obtained as off-white solid (FA salt). 1H NMR (400 MHz, methanol-d4) δ=7.56-7.48 (m, 1H), 7.15 (t, J=9.6 Hz, 1H), 6.97 (s, 2H), 6.73 (s, 1H), 5.46-5.40 (m, 0.5H), 5.29 (br s, 0.5H), 5.05-4.94 (m, 1H), 4.77 (br s, 1H), 4.59-4.52 (m, 2H), 4.30-4.08 (m, 6H), 4.08-4.00 (m, 2H), 3.92-3.82 (m, 1H), 3.74-3.63 (m, 2H), 3.60-3.47 (m, 4H), 3.43-3.35 (m, 4H), 3.21 (br s, 2H), 3.16 (br s, 2H), 2.80-2.70 (m, 1H), 2.30 (br dd, J=9.2, 12.6 Hz, 2H), 2.22-2.16 (m, 1H), 2.13-2.00 (m, 4H), 1.97-1.90 (m, 1H), 1.33 (br s, 1H), 1.30 (br s, 1H), 1.12 (br t, J =7.2 Hz, 3H); 19F NMR (400 MHz, methanol-d4) δ=−122.969, -173.801; LCMS (ESI, M+1): m/z=754.4.

### Example 274

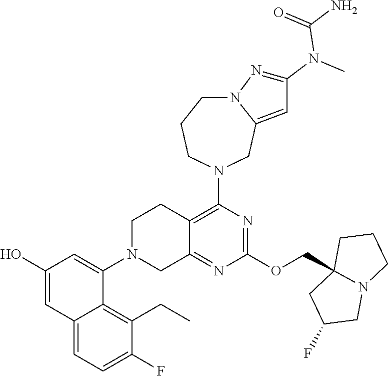 [SMILES: CCc1c(F)ccc2cc(O)cc(N3CCc4c(nc(OC[C@@]56CCCN5C[C@H](F)C6)nc4N4CCCn5nc(N(C)C(N)=O)cc5C4)C3)c12]

### 1-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-1-methylurea

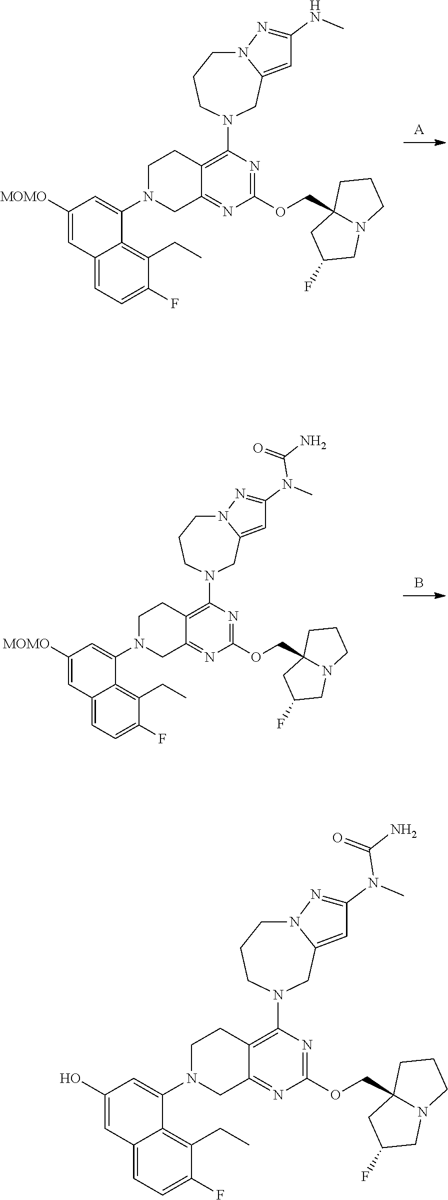

Step A. 1-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-1-methylurea: To a mixture of 5-(7-(8-ethyl-7-fluoro-3-(methoxymethoxy)naphthalen-1-yl)-2-(((2R,7aS)-2-fluorotetrahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepin-2-amine (100 mg, 1.0 equiv) in methanol (2 mL) was added sodium cyanate (28.3 mg, 3.0 equiv) in AcOH (2.10 g, 241 equiv). The reaction was stirred at 50° C. for 2 hours. The mixture was concentrated, dissolved in methanol (2 mL), neutralized with solid NaHCO3, and concentrated. The residue was diluted with water (5 mL), extracted with EtOAc (3×5 mL), dried over anhydrous sodium sulfate, and concentrated to afford the title compound (100 mg, 92% yield) as yellow oil; 1H NMR (400 MHz, chloroform-d) δ=7.61-7.52 (m, 1H), 7.24-7.16 (m, 2H), 7.05-7.00 (m, 1H), 5.95-5.89 (m, 1H), 5.44-5.20 (m, 3H), 4.95-4.77 (m, 2H), 4.68-4.52 (m, 1H), 4.46-4.30 (m, 2H), 4.27-4.16 (m, 2H), 4.10-3.99 (m, 1H), 3.92-3.73 (m, 2H), 3.72-3.66 (m, 3H), 3.63-3.56 (m, 1H), 3.55-3.51 (m, 3H), 3.51-3.47 (m, 1H), 3.45-3.31 (m, 2H), 3.28-3.23 (m, 3H), 3.23-3.10 (m, 2H), 3.08-2.97 (m, 1H), 2.69-2.32 (m, 2H), 2.32-2.22 (m, 2H), 2.06-1.98 (m, 4H), 1.18-1.03 (m, 3H); LCMS (ESI, M+1): m/z=732.6.

Step B. 1-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-1-methylurea: To a mixture of 1-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-1-methylurea (90.0 mg, 1.0 equiv) in ACN (230 μL) was added HCl•MeOH (4 M, 461 μL, 15 equiv). The reaction was stirred at 20° C. for 1 hour. The mixture was concentrated, dissolved in methanol (1 mL), neutralized with solid NaHCO₃, filtered, purified by prep-HPLC [Phenomenex luna 150×25 mm×10 μm; A: water (FA), B: ACN; B %: 18%-48% over 10 min] and lyophilized to afford the title compound (30.5 mg, 34% yield, 0.34 equiv FA) as orange solid. ¹H NMR (400 MHz, methanol-d4) δ=7.55-7.47 (m, 1H), 7.19-7.10 (m, 1H), 7.01-6.92 (m, 2H), 6.16-6.08 (m, 1H), 5.44-5.26 (m, 1H), 4.98-4.93 (m, 1H), 4.83-4.77 (m, 1H), 4.47-4.33 (m, 2H), 4.26-4.20 (m, 1H), 4.19-4.12 (m, 2H), 4.11-4.04 (m, 1H), 4.03-3.94 (m, 1H), 3.73-3.64 (m, 1H), 3.57-3.50 (m, 1H), 3.49-3.36 (m, 5H), 3.25-3.22 (m, 3H), 3.21-3.07 (m, 3H), 2.80-2.71 (m, 1H), 2.44-2.25 (m, 3H), 2.20-2.13 (m, 1H), 2.12-2.01 (m, 3H), 2.00-1.86 (m, 1H), 1.15-1.06 (m, 3H); ¹⁹F NMR (400 MHz, methanol-d4) δ=−122.955, -173.765; LCMS (ESI, M+1): m/z=688.0.

### Example 275

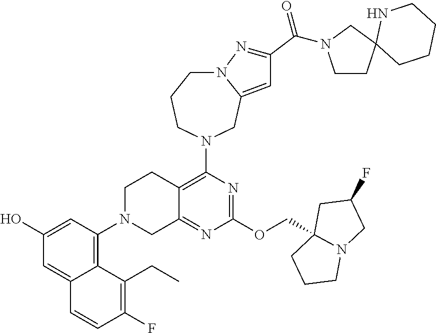 [SMILES: CCc1c(F)ccc2cc(O)cc(N3CCc4c(nc(OC[C@@]56CCCN5C[C@H](F)C6)nc4N4CCCn5nc(C(=O)N6CCC7(CCCCN7)C6)cc5C4)C3)c12]

(5-(7-(8-ethyl-7-fluoro-3-hydroxynaphthalen-1-yl)-2(((2R,7as)-2-flurohexahydro-1H-pyrrolizin-7a-yl)methoxy)-5,6,7,8-tetrahydropyrido[3,4-d]pyrimidin-4yl)-5,6,7,8-tetrahydro-4H-pyrazolo[1,5-a][1,4]diazepin-2-yl)(2,6-diazaspiro[4.5]decan-2yl)methanone

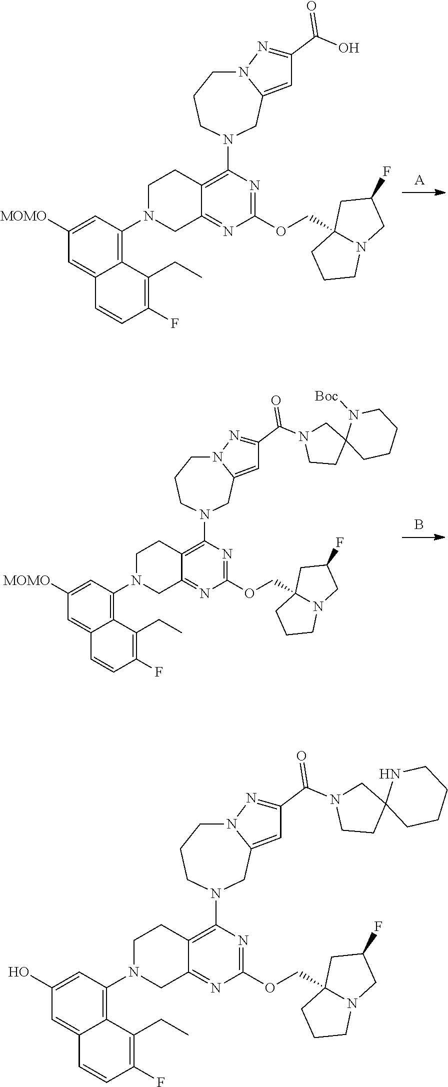

Synthesized according to Example 233 except that HCl instead of TFA was used in the last step. The title compound was obtained as off-white solid (FA salt). 1H NMR (400 MHz, methanol-d4) δ=7.56-7.48 (m, 1H), 7.18-7.11 (m, 1H), 7.00-6.91 (m, 2H), 6.77-6.70 (m, 1H), 5.45-5.26 (m, 1H), 5.06-4.96 (m, 2H), 4.61-4.53 (m, 2H), 4.47-4.29 (m, 1H), 4.28-4.12 (m, 4H), 4.11-3.97 (m, 3H), 3.93-3.81 (m, 1H), 3.76-3.61 (m, 2H), 3.60-3.51 (m, 1H), 3.46-3.37 (m, 4H), 3.24-3.01 (m, 5H), 2.79-2.68 (m, 1H), 2.44-2.24 (m, 4H), 2.22-2.15 (m, 2H), 2.12-2.01 (m, 3H), 2.01-1.89 (m, 1H), 1.86-1.65 (m, 6H), 1.17-1.07 (m, 3H); 19F NMR (400 MHz, methanol-d4) δ=−122.940, -173.652; LCMS (ESI, M+1). m/z=782.5.

### Example 276

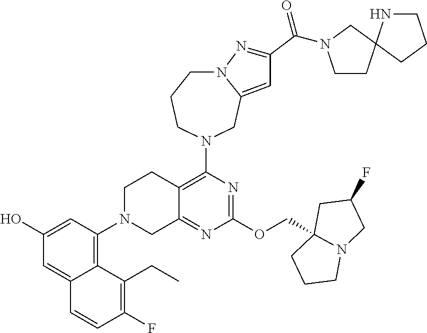 [SMILES: CCc1c(F)ccc2cc(O)cc(N3CCc4c(nc(OC[C@@]56CCCN5C[C@H](F)C6)nc4N4CCCn5nc(C(=O)N6CCC7(CCCN7)C6)cc5C4)C3)c12]

### (5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(1,7-diazaspiro[4.4]nonan-7-yl)methanone

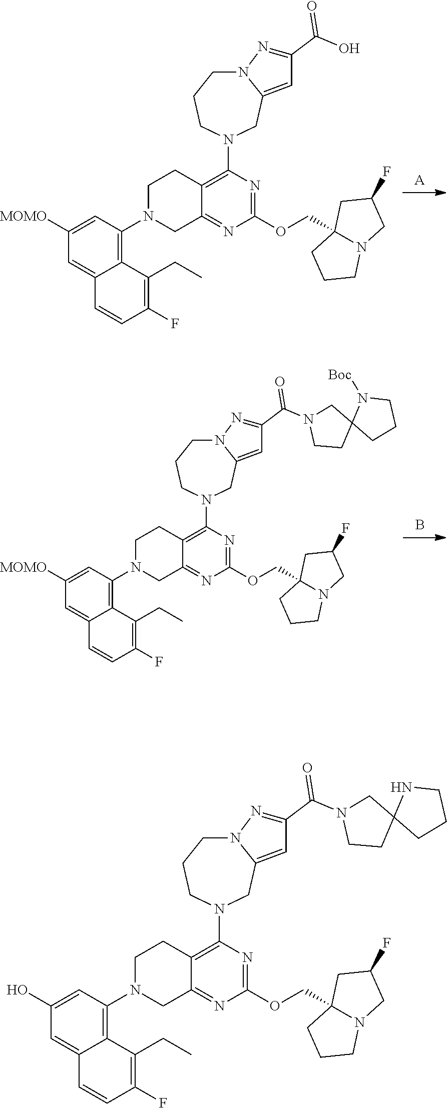

Synthesized according to Example 233 except that HCl instead of TFA was used in the last step. The title compound was obtained as yellow solid (FA salt). 1H NMR (400 MHz, methanol-d4) δ=7.56-7.48 (m, 1H), 7.19-7.11 (m, 1H), 7.00-6.93 (m, 2H), 6.78-6.71 (m, 1H), 5.46-5.26 (m, 1H), 5.09-4.98 (m, 2H), 4.62-4.51 (m, 2H), 4.45-4.30 (m, 1H), 4.29-4.11 (m, 4H), 4.09-4.00 (m, 2H), 3.99-3.74 (m, 2H), 3.74-3.60 (m, 2H), 3.59-3.46 (m, 2H), 3.45-3.35 (m, 4H), 3.26-3.17 (m, 2H), 3.16-3.09 (m, 1H), 2.79-2.69 (m, 1H), 2.45-2.26 (m, 4H), 2.24-2.01 (m, 10H), 2.00-1.87 (m, 1H), 1.18-1.07 (m, 3H); 19F NMR (400 MHz, methanol-d4) δ=−122.940, -173.532; LCMS (ESI, M+1): m/z=768.9.

### Example 277

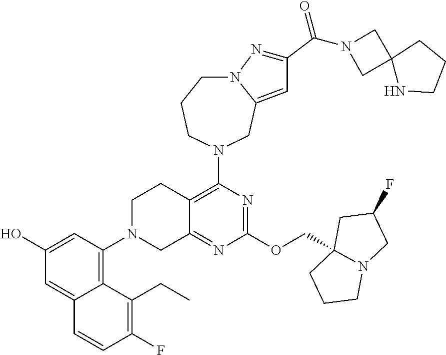 [SMILES: CCc1c(F)ccc2cc(O)cc(N3CCc4c(nc(OC[C@@]56CCCN5C[C@H](F)C6)nc4N4CCCn5nc(C(=O)N6CC7(CCCN7)C6)cc5C4)C3)c12]

### (5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(2,5-diazaspiro[3.4]octan-2-yl)methanone

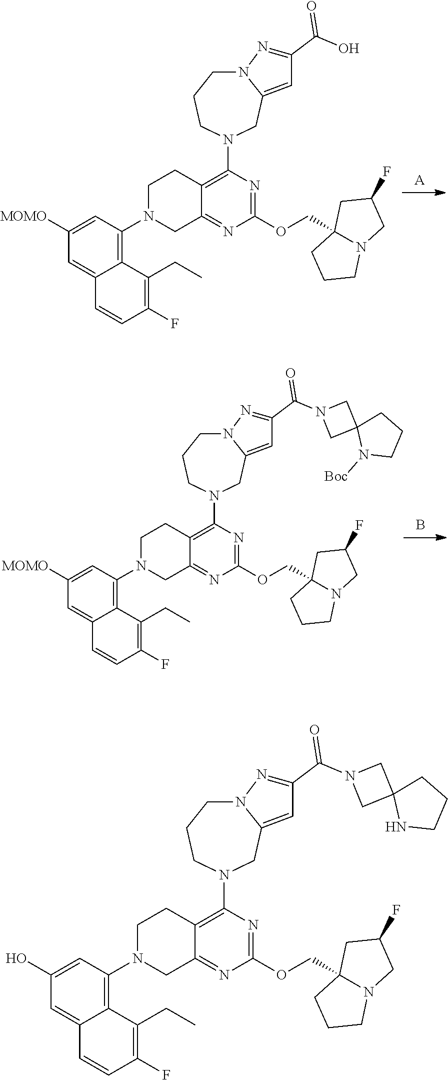

Synthesized according to Example 233 except that HCl instead of TFA was used in the last step. The title compound was obtained as yellow solid (FA salt). 1H NMR (400 MHz, methanol-d4) δ=7.52 (dd, J=6.0, 9.2 Hz, 1H), 7.15 (t, J=9.2 Hz, 1H), 6.98 (s, 2H), 6.74 (s, 1H), 5.53-5.32 (m, 1H), 4.97 (br d, J=9.6 Hz, 2H), 4.70-4.59 (m, 2H), 4.58-4.47 (m, 2H), 4.37-4.15 (m, 5H), 4.05 (br d, J=17.6 Hz, 2H), 3.67 (br d, J=17.2 Hz, 1H), 3.63-3.47 (m, 4H), 3.37 (br dd, J=5.6, 7.2 Hz, 2H), 3.29-3.17 (m, 3H), 3.14 (dt, J=2.8, 7.2 Hz, 2H), 2.75 (br d, J=14.4 Hz, 1H), 2.59-2.35 (m, 2H), 2.34-2.24 (m, 2H), 2.22-2.10 (m, 4H), 2.10-1.88 (m, 4H), 1.21-1.04 (m, 3H); 19F NMR (400 MHz, methanol-d4) δ=−122.910, -173.743, LCMS (ESI, M+1): m/z=754.1.

### Example 278

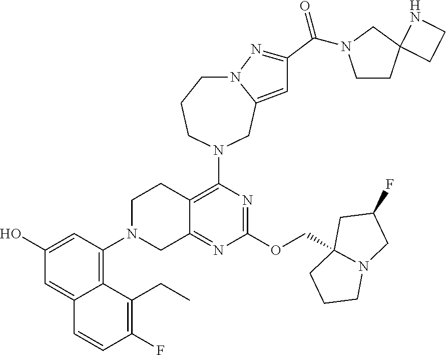 [SMILES: CCc1c(F)ccc2cc(O)cc(N3CCc4c(nc(OC[C@@]56CCCN5C[C@H](F)C6)nc4N4CCCn5nc(C(=O)N6CCC7(CCN7)C6)cc5C4)C3)c12]

### (5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(1,6-diazaspiro[3.4]octan-6-yl)methanone

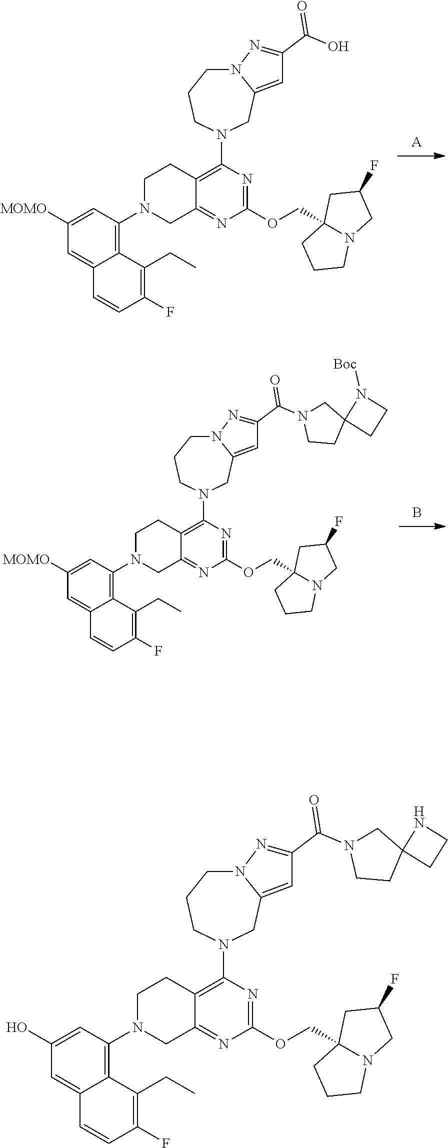

Synthesized according to Example 233 except that HCl instead of TFA was used in the last step. The title compound was obtained as yellow solid (FA salt). 1H NMR (400 MHz, methanol-d4) δ=7.52 (dd, J=5.6, 8.8 Hz, 1H), 7.14 (t, J=9.2 Hz, 1H), 6.97 (s, 2H), 6.80-6.70 (m, 1H), 5.48-5.25 (m, 1H), 5.06-4.97 (m, 1H), 4.83-4.75 (m, 1H), 4.66-4.49 (m, 2H), 4.35-4.11 (m, 4H), 4.10-3.72 (m, 6H), 3.70-3.50 (m, 3H), 3.48-3.34 (m, 5H), 3.26-3.08 (m, 3H), 2.80-2.50 (m, 4H), 2.46-2.16 (m, 5H), 2.14-1.90 (m, 4H), 1.12 (br t, J=7.2 Hz, 3H), 19F NMR (400 MHz, methanol-d4) δ=−122.947, -173.375, LCMS (ESI, M+1): m/z=754.1.

### Example 279

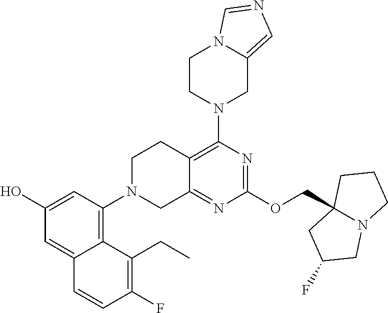 [SMILES: CCc1c(F)ccc2cc(O)cc(N3CCc4c(nc(OC[C@@]56CCCN5C[C@H](F)C6)nc4N4CCn5cncc5C4)C3)c12]

### 4-(4-(5,6-dihydroimidazo[1,5-a]pyrazin-7(8H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

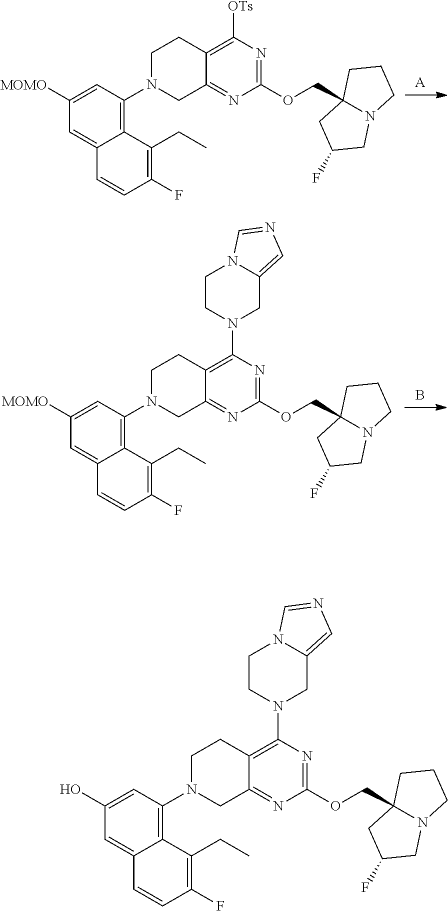

Synthesized according to Example 248. The title compound was obtained as brown solid (FA salt). 1H NMR (400 MHz, METHANOL-d4) δ=7.78-7.68 (m, 1H), 7.58-7.47 (m, 1H), 7.22-7.12 (m, 1H), 7.03-6.96 (m, 2H), 6.89 (br s, 1H), 5.60-5.38 (m, 1H), 4.67-4.55 (m, 1H), 4.52-4.33 (m, 3H), 4.32-4.20 (m, 2H), 4.18-4.07 (m, 1H), 3.90-3.67 (m, 5H), 3.60-3.47 (m, 1H), 3.45-3.35 (m, 2H), 3.28-3.17 (m, 3H), 2.83-2.73 (m, 1H), 2.64-2.40 (m, 2H), 2.37-2.00 (m, 5H), 1.17-1.06 (m, 3H); LCMS (ESI, M+1): m/z=602.4.

### Example 280

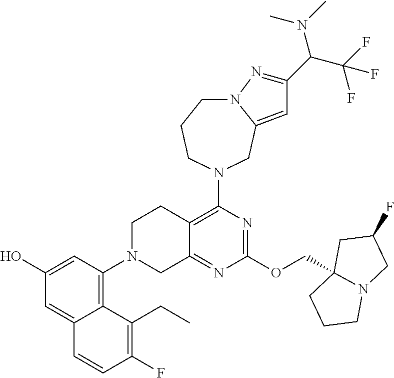 [SMILES: CCc1c(F)ccc2cc(O)cc(N3CCc4c(nc(OC[C@@]56CCCN5C[C@H](F)C6)nc4N4CCCn5nc(C(N(C)C)C(F)(F)F)cc5C4)C3)c12]

### 4-(4-(2-(1-(dimethylamino)-2,2,2-trifluoroethyl)-7,8-dihydro-4H-pyrazolo[1,5-a][1,4]diazepin-5(6H)-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-ol

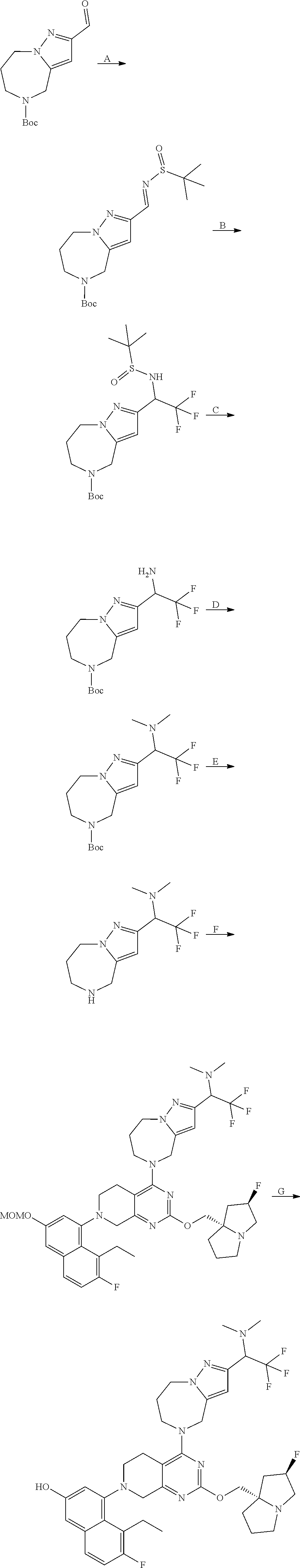

Step A tert-butyl(E)-2-(((tert-butylsulfinyl)imino)methyl)-7,8-dihydro-4H-pyrazolo[1,5-a][1,4]diazepine-5(6H)-carboxylate: To a solution of tert-butyl 2-formyl-7,8-dihydro-4H-pyrazolo[1,5-a][1,4]diazepine-5(6H)-carboxylate (200 mg, 1.0 equiv) in THE (8.0 mL) was added 2-methylpropane-2-sulfinamide (184 mg, 2.0 equiv). The mixture was stirred at 0° C. for 6 hours. The reaction was concentrated under reduced pressure to give a residue. The residue was purified with column chromatography [SiO2, petroleum ether/ethyl acetate=5/1 to 3/1] to afford the title compound (200 mg, 68% yield, 95% purity). LCMS (ESI, M+1): m/z=369.1

Step B: tert-butyl2-(1-((tert-butylsulfinyl)amino)-2,2,2-trifluoroethyl)-7,8-dihydro-4H-pyrazolo[1,5-a][1,4]diazepine-5(6H)-carboxylate: To a solution of tert-butyl (E)-2-(((tert-butylsulfinyl)imino)methyl)-7,8-dihydro-4H-pyrazolo[1,5-a][1,4]diazepine-5(6H)-carboxylate (300 mg, 1.0 equiv) in THE (6.0 mL) was cooled to −78° C. t-BuOK (1.00 M, 2.44 mL, 3.0 equiv) and TMSCF3 (347 mg, 3.0 equiv) were then sequentially added to the reaction and allowed to slowly warm to 0° C. for 2 hours. The reaction was partitioned between ethyl acetate (20.0 mL) and water (10.0 mL). The residue was purified with prep-TLC [ethylacetate/petroleum =1/1] to afford the title compound (160 mg, 40% yield, 90% purity), LCMS (ESI, M+1): m/z=439.3

Step C: tert-butyl 2-(1-amino-2,2,2-trifluoroethyl)-7,8-dihydro-4H-pyrazolo[1,5-a][1,4]diazepine-5(6H)-carboxylate: To a solution of tert-butyl 2-(1-((tert-butylsulfinyl)amino)-2,2,2-trifluoroethyl)-7,8-dihydro-4H-pyrazolo[1,5-a][1,4]diazepine-5(6H)-carboxylate (450 mg, 1.00 equiv) in THF (1.00 mL) and water (0.20 mL) was added (1-(pyrrolidin-1-ylmethyl)cyclopropyl)methanol (43.4 mg, 0.50 equiv). The mixture was stirred at 50° C. for 2 hours. The reaction was partitioned between ethyl acetate (20.0 mL) and a saturated solution of sodium thiosulfate (10.0 mL). The organic phase was separated and concentrated to give a residue to afford the title compound (100 mg, 70% yield, 92% purity), LCMS (ESI, M+1): m/z=335.1

Step D: tert-butyl 2-(1-(dimethylamino)-2,2,2-trifluoroethyl)-7,8-dihydro-4H-pyrazolo[1,5-a][1,4]diazepine-5(6H)-carboxylate: To a solution of tert-butyl 2-(1-amino-2,2,2-trifluoroethyl)-7,8-dihydro-4H-pyrazolo[1,5-a][1,4]diazepine-5(6H)-carboxylate (100 mg, 1 equiv), paraformaldehyde (1.21 g), and acetic acid (8.98 mg) in DCE (1.00 ml) was added NaBH3CN (94.0 mg). The mixture was stirred at 40° C. for 4 hours. The reaction was partitioned between ethyl acetate (20.0 mL) and water (10.0 mL). The residue was purified with prep-TLC [SiO2, petroleum ether/ethyl acetate=0/1) to afford the title compound (60.0 mg, 55% yield, 70% purity), LCMS (ESI, M+1): m/z=363.2

Step E: 2,2,2-trifluoro-N,N-dimethyl-1-(5,6,7,8-tetrahydro-4H-pyrazolo[1,5-a][1,4]diazepin-2-yl)ethan-1-amine: To a solution of tert-butyl 2-(1-(dimethylamino)-2,2,2-trifluoroethyl)-7,8-dihydro-4H-pyrazolo[1,5-a][1,4]diazepine-5(6H)-carboxylate (50.0 mg, 1.0 equiv) in MeCN (0.50 mL) was added HCl/dioxane (0.50 mL). The mixture was stirred at 20° C. for 1 hour. The reaction was concentrated to afford the title compound (30.0 mg, 83% yield, 90% purity), LCMS (ESI, M+1): m/z=263.1

The last two steps were performed according to Example 248. The title compound was obtained as yellow solid (FA salt). 1H NMR (400 MHz, METHANOL-d4) δ=8.54-8.51 (m, 1H), 7.51 (dd, J=5.8, 8.8 Hz, 1H), 7.15 (t, J=9.6 Hz, 1H), 7.01-6.95 (m, 2H), 6.35-6.28 (m, 1H), 5.43-5.23 (m, 1H), 5.16-4.99 (m, 1H), 4.99-4.89 (m, 1H), 4.85-4.73 (m, 1H), 4.60-4.45 (m, 2H), 4.32-4.18 (m, 3H), 4.17-4.10 (m, 1H), 4.05 (br dd, J=8.7, 18.4 Hz, 1H), 3.98-3.87 (m, 1H), 3.65 (br dd, J=10.1, 17.6 Hz, 1H), 3.57-3.48 (m, 1H), 3.46-3.34 (m, 4H), 3.29-3.23 (m, 1H), 3.21-3.15 (m, 1H), 3.13-3.05 (m, 1H), 2.74 (br d, J=16.0 Hz, 1H), 2.43-2,33 (m, 1H), 2.30 (d, J=7.2 Hz, 7H), 2.26-2.20 (m, 1H), 2.13 (br d, J=10.8 Hz, 1H), 2.10-1.99 (m, 3H), 1.98-1.87 (m, 1H), 1.16-1.06 (m, 3H); LCMS (ESI, M+1): m/z=741.4

### Example 281

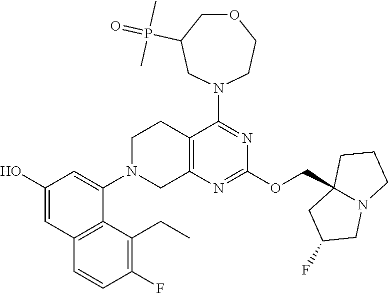 [SMILES: CCc1c(F)ccc2cc(O)cc(N3CCc4c(nc(OC[C@@]56CCCN5C[C@H](F)C6)nc4N4CCOCC(P(C)(C)=O)C4)C3)c12]

### (4-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,4-oxazepan-6-yl)dimethylphosphine oxide

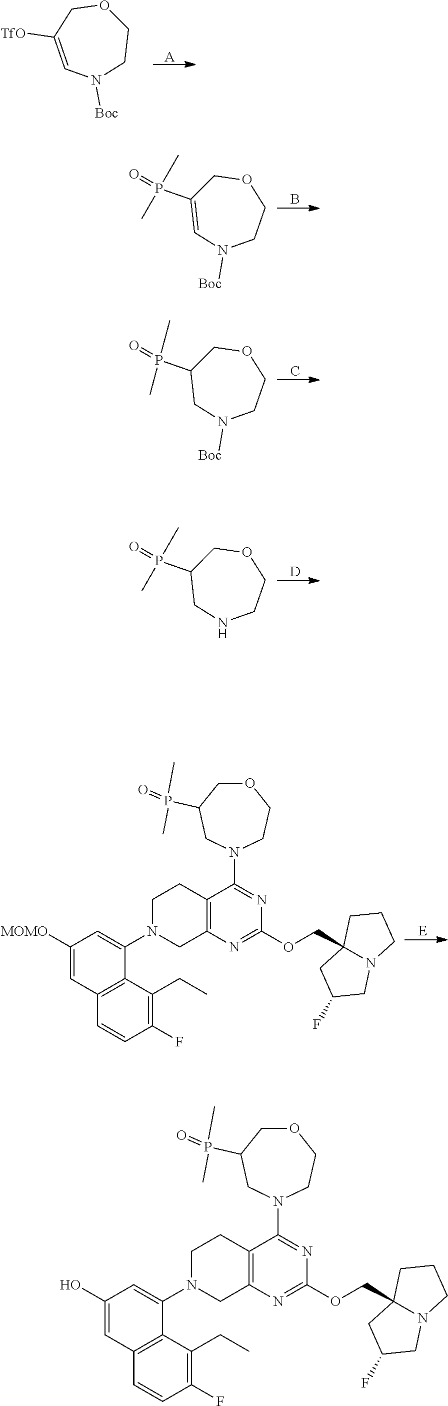

Step A. tert-butyl 6-(dimethylphosphoryl)-2,3-dihydro-1,4-oxazepine-4(7H)-carboxylate: A mixture of tert-butyl 6-(((trifluoromethyl)sulfonyl)oxy)-2,3-dihydro-1,4-oxazepine-4(7H)-carboxylate (350 mg, 1 equiv), methylphosphonoylmethane (86.5 mg, 1.1 equiv), Pd(PPh3)4 (58.2 mg, 0.05 equiv), and Et3N (204 mg, 2.0 equiv) in MeCN (10 mL) was stirred at 90° C. for 10 hours under N2 atmosphere. The reaction was concentrated and purified by prep-HPLC (column: Waters Xbridge 150×25 mm×5 um; mobile phase: [water (ammonia hydroxide v/v)-ACN]; B %: 12%-42%,9 min) to afford the title compound. 1H NMR (400 MHz, CHLOROFORM-d) δ=7.51 (d, J=15.6 Hz, 1H), 4.35 (br d, J=7.6 Hz, 2H), 3.99-3.84 (m, 4H), 1.59 (s, 3H), 1.55 (s, 3H), 1.52 (s, 9H)

Step B. tert-butyl 6-(dimethylphosphoryl)-1,4-oxazepane-4-carboxylate: A mixture of tert-butyl 6-(dimethylphosphoryl)-2,3-dihydro-1,4-oxazepine-4(7H)-carboxylate (100 mg, 1.0 equiv) and Pd/C (50 mg, 60% purity, 1.0 equiv) in MeOH (1.0 mL) was degassed and purged with H2 3 times, and then the mixture was stirred at 25° C. for 2 hours under H2 atmosphere. The reaction was filtered with MeOH (20 mL) and concentrated under reduced pressure to afford the title compound (100 mg) as a white solid.

Step C. dimethyl(1,4-oxazepan-6-yl)phosphine oxide: To a solution of tert-butyl 6-(dimethylphosphoryl)-1,4-oxazepane-4-carboxylate (100 mg, 1.0 equiv) in DCM (2.0 mL) was added TFA (411 mg, 10.0 equiv). The mixture was stirred at 25° C. for 1 hour. The reaction was concentrated under reduced pressure to afford the title compound (50 mg) as a yellow oil.

The last two steps were performed according to Example 248. The title compound was obtained as pink solid. 1H NMR (400 MHz, DMSO-d6) δ=9.70-9.65 (m, 1H), 7.59 (dd, J=6.2, 8.5 Hz, 1H), 7.24 (t, J=9.2 Hz, 1H), 7.03-6.93 (m, 2H), 5.34-5.15 (m, 1H), 4.53-4.22 (m, 1H), 4.06 (br d, J=16 Hz, 1H), 3.95-3.82 (m, 6H), 3.75-3.57 (m, 3H), 3.50-3.39 (m, 2H), 3.15-3.01 (m, 4H), 2.98 (s, 1H), 2.80 (br d, J=7.6 Hz, 1H), 2.67 (br dd, J=2, 3.7 Hz, 2H), 2.16-1.88 (m, 4H), 1.87-1.66 (m, 4H), 1.49-1.42 (m, 6H), 1.08-1.01 (m, 3H), LCMS (ESI, M+1): m/z=656.1.

### Example 282

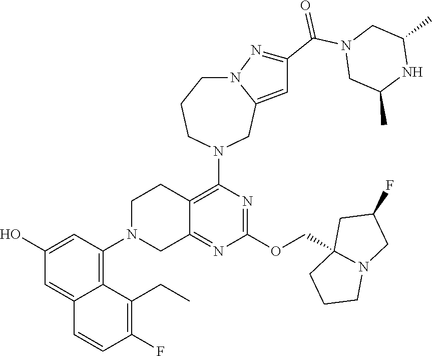 [SMILES: CCc1c(F)ccc2cc(O)cc(N3CCc4c(nc(OC[C@@]56CCCN5C[C@H](F)C6)nc4N4CCCn5nc(C(=O)N6C[C@H](C)N[C@@H](C)C6)cc5C4)C3)c12]

### ((3S,5S)-3,5-dimethylpiperazin-1-yl)(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

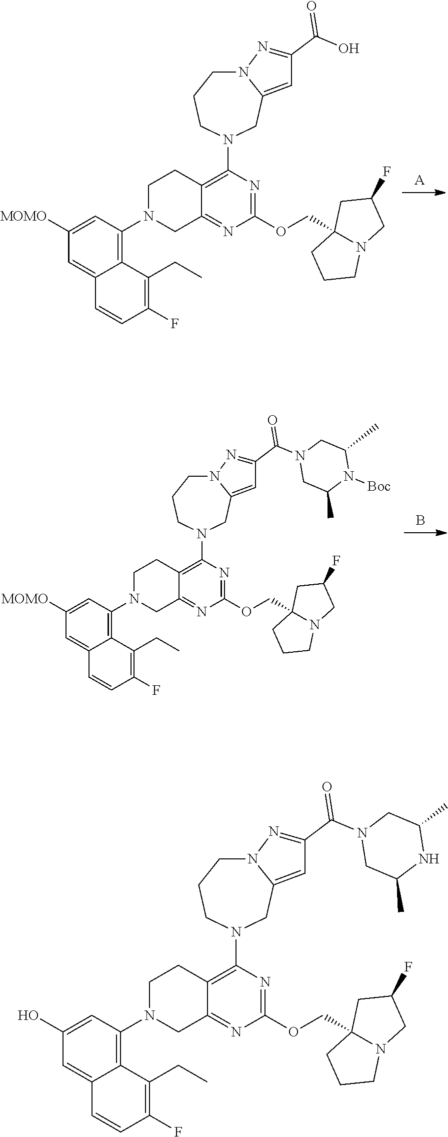

Synthesized according to Example 233 except that HCl instead of TFA was use in the last step. The title compound was obtained as white solid (FA salt). 1H NMR (400 MHz, methanol-d4) δ=7.52 (dd, J=5.6, 8.8 Hz, 1H), 7.14 (t, J=9.2 Hz, 1H), 6.97 (s, 2H), 6.70 (s, 1H), 5.50-5.31 (m, 1H), 5.06-4.87 (m, 2H), 4.54 (br d, J=7.2 Hz, 2H), 4.39-4.18 (m, 4H), 4.17-3.97 (m, 4H), 3.92 (br dd, J=3.2, 10.4 Hz, 1H), 3.68 (br d, J=17.6 Hz, 1H), 3.60-3.47 (m, 6H), 3.43-3.34 (m, 2H), 3.24-3.13 (m, 3H), 2.75 (br d, J=14.0 Hz, 1H), 2.52-2.20 (m, 4H), 2.17-1.97 (m, 4H), 1.29 (br s, 6H), 1.11 (t, J=7.2 Hz, 3H); 19F NMR (400 MHz, methanol-d4) δ=−122.947, -173.718; LCMS (ESI, M+1): m/z=756.6.

### Example 283

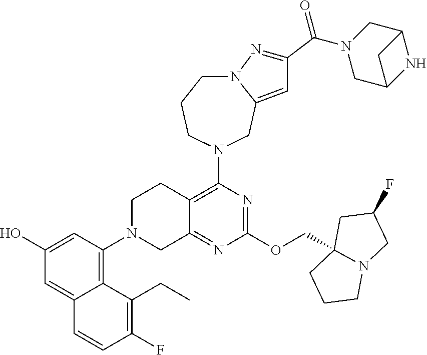 [SMILES: CCc1c(F)ccc2cc(O)cc(N3CCc4c(nc(OC[C@@]56CCCN5C[C@H](F)C6)nc4N4CCCn5nc(C(=O)N6CC7CC(C6)N7)cc5C4)C3)c12]

### 3,6-diazabicyclo[3.1.1]heptan-3-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5 6,7,8-tetrahydro-4Hi-pyrazolo[1,5-a][1,4]diazepin-2-yl)methanone

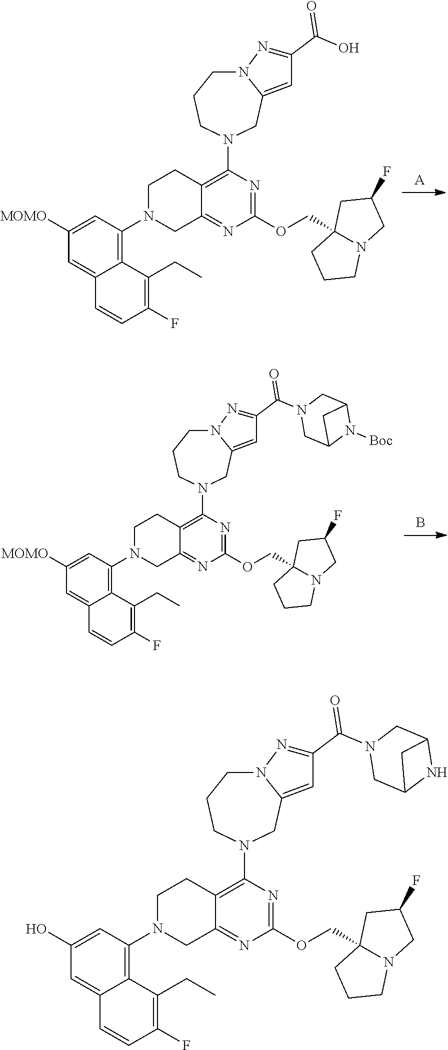

Synthesized according to Example 233 except that HCl instead of TFA was used in the last step. The title compound was obtained as white solid (FA salt). 1H NMR (400 MHz, methanol-d4) δ=7.51 (dd, J=5.2, 8.4 Hz, 1H), 7.14 (t, J=9.2 Hz, 1H), 6.97 (d, J=2.4 Hz, 2H), 6.81-6.73 (m, 1H), 5.49-5.26 (m, 1H), 5.07-4.95 (m, 1H), 4.90 (br dd, J=3.6, 6.4 Hz, 1H), 4.68-4.62 (m, 1H), 4.57 (br d, J=2.8 Hz, 2H), 4.44-4.33 (m, 1H), 4.28-4.13 (m, 6H), 4.11-4.01 (m, 2H), 3.98-3.87 (m, 1H), 3.68 (br dd, J=2.4, 17.2 Hz, 1H), 3.55 (br d, J=9.2 Hz, 1H), 3.50-3.36 (m, 5H), 3.23-3.07 (m, 3H), 2.97-2.87 (m, 1H), 2.74 (br d, J=14.4 Hz, 1H), 2.48-2.15 (m, 4H), 2.12-1.90 (m, 4H), 1.86-1.77 (m, 1H), 1.16-1.05 (m, 3H), 19F NMR (400 MHz, methanol-d4) δ=−122.939, -173.651, LCMS (ESI, M+1): m/z=740.6.

### Example 284

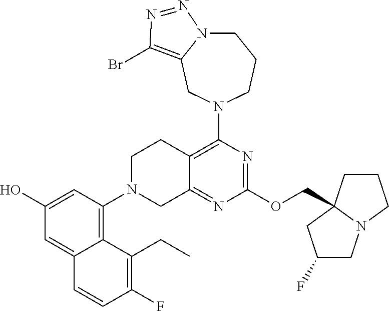 [SMILES: CCc1c(F)ccc2cc(O)cc(N3CCc4c(nc(OC[C@@]56CCCN5C[C@H](F)C6)nc4N4CCCn5nnc(Br)c5C4)C3)c12]

### 4-(4-(3-bromo-7,8-dihydro-4H-[1,2,3]triazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

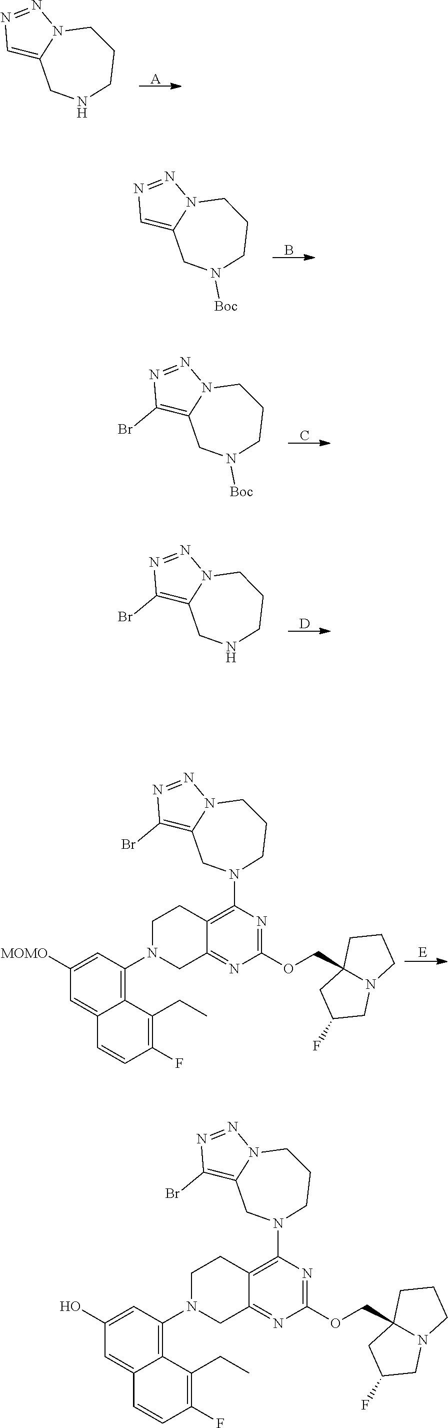

Step A. tert-butyl 7,8-dihydro-4H-[1,2,3]triazolo[1,5-a][1,4]diazepine-5)(6( )-carboxylate: To a mixture of 5,6,7,8-tetrahydro-4H-[1,2,3]triazolo[1,5-a][1,4]diazepine (1.00 g, 1.0 equiv) and Boc2O (3.16 g, 2.0 equiv) in DCM (8.0 mL) were added DMAP (88.4 mg, 0.10 equiv) and TEA (2.20 g, 3.0 equiv). The reaction was stirred at 20° C. for 12 hours. The mixture was filtered and purified by reversed phase HaPLC (e2o FA condition) to afford the title compound (1.09 g, 63 yield) as yellow solid; LCMS (ESI, M1): m/z=239.0.

Step B. tert-butyl 3-bromo-7,8-dihydro-4H-[1,2,3]triazolo[1,5-a][1,4]diazepine-5(6H)-carboxylate: To a mixture of tert-butyl 7,8-dihydro-4H-[1,2,3]triazolo[1,5-a][1,4]diazepine-5(6H)-carboxylate (650 mg, 1.0 equiv) in MeCN (6.0 mL) was added NBS (1.46 g, 3.0 equiv). The reaction was stirred at 90° C. for 4 hours. The reaction was diluted with water (80 mL) and extracted with ethyl acetate (3×30 mL). The combined organic layers were washed with brine (2×20 mL), dried over anhydrous sodium sulfate, concentrated, and purified by column chromatography (SiO2, Petroleum ether/Ethyl acetate=5/1 to 1/1) and reversed phase HPLC [water (FA, 0.1%)/acetonitrile] to afford the title compound (640 mg, 72% yield) as yellow oil. LCMS (ESI, M+1, M+3): m/z=316.9, 318.9.

Step C. 3-bromo-5,6,7,8-tetrahydro-4H-[1,2,3]triazolo[1,5-a][1,4]diazepine: To a solution of tert-butyl 3-bromo-7,8-dihydro-4H-[1,2,3]triazolo[1,5-a][1,4]diazepine-5(6H)-carboxylate (100 mg, 1.0 equiv) in MeCN (1.0 mL) was added HCl/dioxane (4 M, 1.0 mL, 13 equiv). The reaction was stirred at 20° C. for 0.5 hours. The mixture was concentrated under vacuum to afford the title compound (110 mg, crude) as white solid and was used into next step without further purification.

The last two steps were performed according to Example 248. The title compound was obtained as off-white solid (FA salt). 1H NMR (400 MHz, METHANOL-d4) δ=7.51 (dd, J=5.6, 8.8 Hz, 1H), 7.15 (t, J=9.2 Hz, 1H), 7.03-6.92 (m, 2H), 5.49-5.27 (m, 1H), 4.92 (br s, 1H), 4.83-4.72 (m, 2H), 4.66-4.58 (m, 1H), 4.24-4.02 (m, 5H), 3.69 (br d, J=18.0 Hz, 1H), 3.55-3.46 (m, 2H), 3.46-3.37 (m, 4H), 3.28-3.12 (m, 3H), 2.71 (br d, J=14.4 Hz, 1H), 2.46-2.24 (m, 3H), 2.23-2.14 (m, 2H), 2.13-2.03 (m, 2H), 2.01-1.89 (m, 1H), 1.11 (t, J=7.2 Hz, 3H); LCMS (ESI, M+1): m/z=695.0, 697.0.

### Example 285

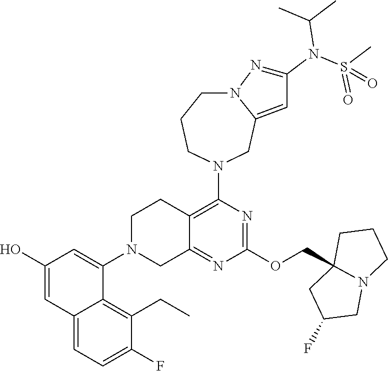 [SMILES: CCc1c(F)ccc2cc(O)cc(N3CCc4c(nc(OC[C@@]56CCCN5C[C@H](F)C6)nc4N4CCCn5nc(N(C(C)C)S(C)(=O)=O)cc5C4)C3)c12]

### N-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-isopropylmethanesulfonamide

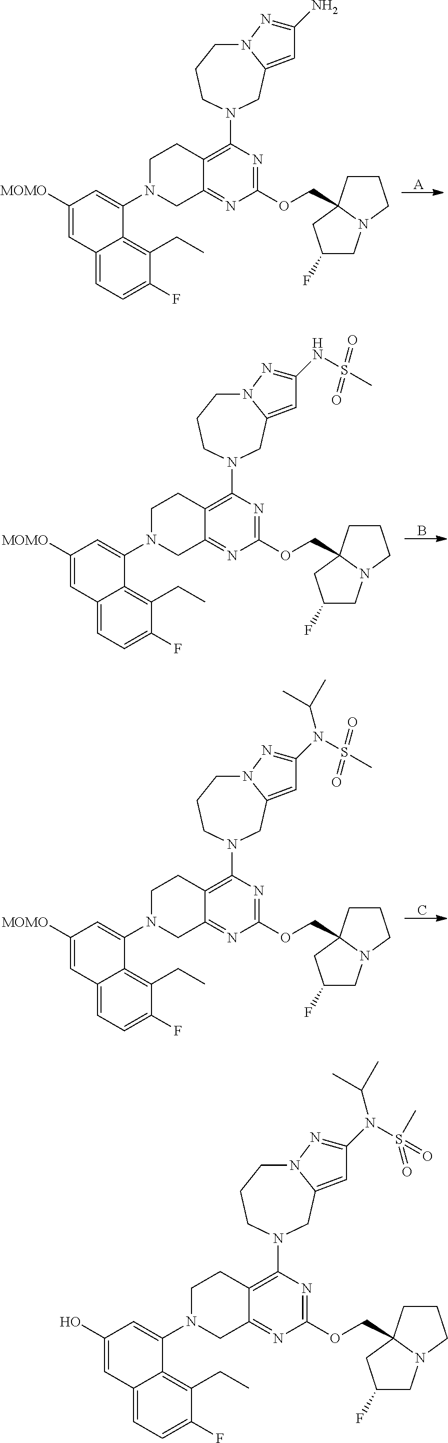

Step A. N-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanesulfonamide: To a mixture of 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-amine (300 mg, 1.0 equiv) in Pyridine (4 mL) was added Ms20 (116 mg, 1.5 equiv) in an ice-bath. The reaction was warmed to 20° C. and stirred for 4 hours. The mixture was diluted with water (10 mL) and extracted with EtOAc (3×20 mL). The combined organic layers were dried over anhydrous sodium sulfate, concentrated, and purified by reversed phase flash chromatography [C18, 0.1% formic acid condition] to afford the title compound (300 mg, 86% yield) as yellow solid; 1H NMR (400 MHz, dimethylsulfoxide-d6) δ=9.74 (br s, 1H), 7.76-7.69 (m, 1H), 7.36-7.28 (m, 2H), 7.17-7.11 (m, 1H), 5.96 (s, 1H), 5.35-5.16 (m, 3H), 4.98-4.64 (m, 2H), 4.35 (br d, J=5.6 Hz, 2H), 3.94-3.75 (m, 4H), 3.64 (br d, J=17.6 Hz, 1H), 3.42 (s, 3H), 3.29-3.26 (m, 3H), 3.19-3.12 (m, 2H), 3.10-3.03 (m, 2H), 3.01-2.94 (m, 4H), 2.83-2.76 (m, 1H), 2.15 (br s, 2H), 2.01 (br s, 2H), 1.93 (br d, J=1.2 Hz, 2H), 1.86-1.66 (m, 4H), 1.07 (t, J=7.2 Hz, 3H); LCMS (ESI, M+1): m/z=753.2.

Step B. N-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-isopropylmethanesulfonamide: To a mixture of N-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanesulfonamide (80 mg, 1.0 equiv) in pyridine (1.57 g, 177 equiv) at 0° C. was added methylsulfonyl methanesulfonate (68 mg, 3.5 equiv). The reaction was stirred at 20° C. for 4 hours. The mixture was filtered and purified by reversed phase flash chromatography [C18, 0.1% formic acid condition] to afford the title compound (50 mg, 54% yield) as orange solid; 1H NMR (400 MHz, chloroform-d) δ=7.60-7.51 (m, 1H), 7.24-7.16 (m, 2H), 7.05-6.98 (m, 1H), 6.18-5.97 (m, 1H), 5.36-5.15 (m, 3H), 4.93-4.53 (m, 2H), 4.52-4.35 (m, 3H), 4.26-4.15 (m, 1H), 4.15-4.04 (m, 1H), 4.03-3.80 (m, 3H), 3.79-3.69 (m, 1H), 3.59-3.51 (m, 3H), 3.50-3.45 (m, 1H), 3.43-3.31 (m, 2H), 3.30-3.20 (m, 2H), 3.18-3.11 (m, 2H), 3.11-3.04 (m, 3H), 3.03-2.89 (m, 1H), 2.69-2.56 (m, 1H), 2.35-2.04 (m, 5H), 2.00-1.79 (m, 3H), 1.73-1.64 (m, 1H), 1.29-1.16 (m, 6H), 1.16-1.08 (m, 3H); 19F NMR (400 MHz, chloroform-d) δ=−119.247, -173.073; LCMS (ESI, M+1): m/z=795.1.

Step C. N-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-isopropylmethanesulfonamide: To a mixture of N-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-isopropylmethanesulfonamide (45 mg, 1.0 equiv) in ACN (106 μL) was added HCl•MeOH (4 M,15 equiv). The mixture was stirred at 20° C. for 1 hour. The reaction was filtered and purified by prep-HPLC [Phenomenex luna C18 150×25 mm×10 μm; A: water (FA), B: ACN; B %: 27%-57% over 10 min] to afford the title compound (7.96 mg, 18% yield, 0.22 equiv FA) as orange solid. 1H NMR (400 MHz, dimethylsulfoxide-d6) δ=9.80-9.55 (m, 1H), 7.64-7.54 (m, 1H), 7.28-7.19 (m, 1H), 7.04-6.93 (m, 2H), 6.15-6.08 (m, 1H), 5.35-5.11 (m, 1H), 4.98-4.84 (m, 1H), 4.79-4.67 (m, 1H), 4.51-4.36 (m, 2H), 4.27-4.13 (m, 1H), 4.09-3.97 (m, 1H), 3.96-3.77 (m, 4H), 3.65-3.54 (m, 1H), 3.45-3.37 (m, 2H), 3.25-3.21 (m, 1H), 3.17-3.11 (m, 1H), 3.10-3.02 (m, 6H), 3.01-2.95 (m, 1H), 2.86-2.76 (m, 1H), 2.70-2.63 (m, 1H), 2.25-2.11 (m, 1H), 2.07-2.01 (m, 1H), 2.00-1.87 (m, 3H), 1.85-1.66 (m, 3H), 1.11-0.98 (m, 9H); 1H NMR (400 MHz, dimethylsulfoxide-d6+deuterium oxide-d2) δ=7.61-7.52 (m, 1H), 7.27-7.19 (m, 1H), 7.03-6.92 (m, 2H), 6.15-6.07 (m, 1H), 5.34-5.12 (m, 1H), 4.94-4.83 (m, 1H), 4.77-4.67 (m, 1H), 4.47-4.33 (m, 2H), 4.24-4.11 (m, 1H), 4.11-4.00 (m, 1H), 3.99-3.77 (m, 4H), 3.59-3.52 (m, 1H), 3.45-3.34 (m, 1H), 3.33-3.19 (m, 2H), 3.19-3.13 (m, 1H), 3.12-3.06 (m, 3H), 3.05-3.04 (m, 3H), 3.03-2.97 (m, 1H), 2.87-2.76 (m, 1H), 2.71-2.62 (m, 1H), 2.25-2.10 (m, 1H), 2.09-2.02 (m, 1H), 2.01-1.86 (m, 3H), 1.86-1.65 (m, 3H), 1.09-0.92 (m, 9H); 19F NMR (400 MHz, dimethylsulfoxide-d6+deuterium oside-d2) δ=−121.213, -171.797; LCMS (ESI, M+1): m/z=751.7.

### Example 286

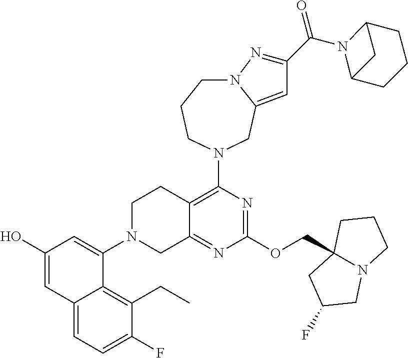 [SMILES: CCc1c(F)ccc2cc(O)cc(N3CCc4c(nc(OC[C@@]56CCCN5C[C@H](F)C6)nc4N4CCCn5nc(C(=O)N6C7CCCC6C7)cc5C4)C3)c12]

### 6-azabicyclo[3.1.1]heptan-6-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

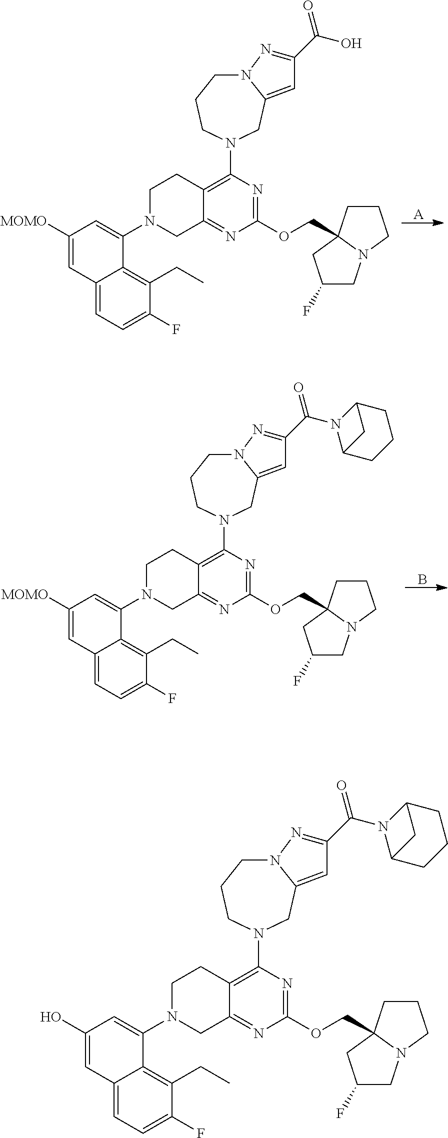

Synthesized according to Example 233 except that HCl instead of TFA was used in the last step. The title compound was obtained as yellow solid. 1HNMR (400 MHz, METHANOL-d4) δ=7.51 (dd, J=5.6, 8.8 Hz, 1H), 7.14 (t, J=9.4 Hz, 1H), 7.01-6.93 (m, 2H), 6.71-6.65 (m, 1H), 5.36-5.16 (m, 1H), 5.01 (td, J=3.2, 6.0 Hz, 2H), 4.56-4.44 (m, 3H), 4.27-4.15 (m, 1H), 4.11-3.96 (m, 4H), 3.71-3.61 (m, 1H), 3.54 (br dd, J=2.4, 8.8 Hz, 1H), 3.44-3.38 (m, 2H), 3.27-3.10 (m, 6H), 3.02-2.92 (m, 1H), 2.78-2.68 (m, 1H), 2.66-2.56 (m, 1H), 2.39-2.18 (m, 4H), 2.15-2.02 (m, 4H), 1.98-1.82 (m, 5H), 1.75-1.64 (m, 2H), 1.17-1.05 (m, 3H); LCMS (ESI, M+1): m/z=739.4

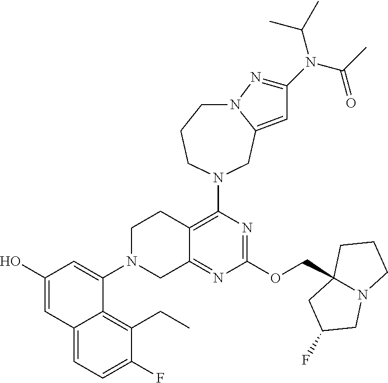 [SMILES: CCc1c(F)ccc2cc(O)cc(N3CCc4c(nc(OC[C@@]56CCCN5C[C@H](F)C6)nc4N4CCCn5nc(N(C(C)=O)C(C)C)cc5C4)C3)c12]

### N-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-i sopropyvlacetamide

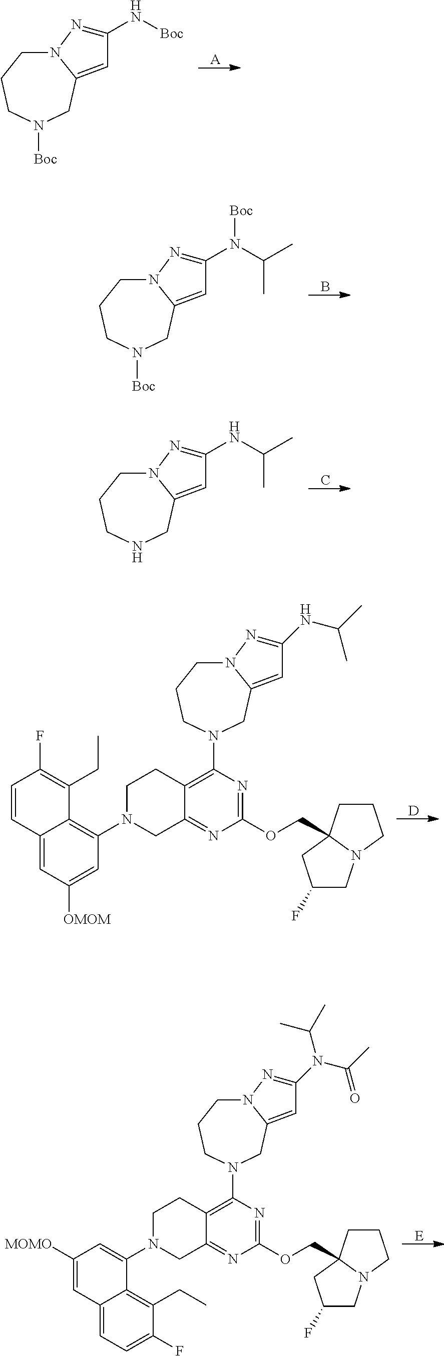

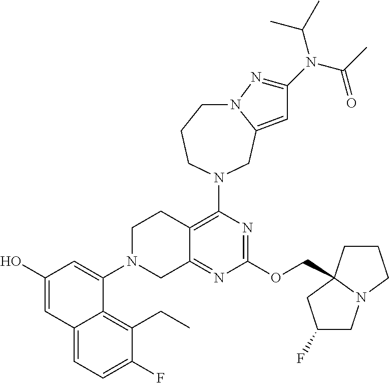 [SMILES: CCc1c(F)ccc2cc(O)cc(N3CCc4c(nc(OC[C@@]56CCCN5C[C@H](F)C6)nc4N4CCCn5nc(N(C(C)=O)C(C)C)cc5C4)C3)c12]

Step A. tert-butyl 2-((tert-butoxycarbonyl)(isopropyl)amino)-7,8-dihydro-4H-pyrazolo[1,5-a][1,4]diazepine-5(6H)-carboxylate: To a mixture of tert-butyl 2-((tert-butoxycarbonyl)amino)-7,8-dihydro-4H-pyrazolo[1,5-a][1,4]diazepine-5(6H)-carboxylate (100 mg, 1.0 equiv) in THF (1 mL) was added NaH (56.7 mg, 60% purity, 5.0 equiv). The reaction was stirred at 25° C. for 1 hour. 2-iodopropane (482 mg, 10 equiv) was added into above mixture. The reaction was stirred at 60° C. for 12 hours. The mixture was quenched with H₂O (3 mL) and extracted with EtOAc (3×5 mL). The combined organic layers were dried over anhydrous sodium sulfate, concentrated and purified by reversed phase flash chromatography [C18, 0.1% formic acid condition] to afford the title compound (50.0 mg, 40% yield) as yellow oil; LCMS (ESI, M+1): m/z=395.2.

Step B. N-isopropyl-5,6,7,8-tetrahydro-4H-pyrazolo[1,5-a][1,4]diazepin-2-amine: To a solution of tert-butyl 2-((tert-butoxycarbonyl)(isopropyl)amino)-7,8-dihydro-4H-pyrazolo[1,5-a][1,4]diazepine-5(6H)-carboxylate (50.0 mg, 1.0 equiv) in MeCN (0.5 mL) was added HCl•dioxane (4.0 M, 2.0 mL). The reaction was stirred at 0° C. for 1 hour. The mixture was adjusted pH to 8 with saturated NaHCO₃ aqueous solution (3.0 mL) and extracted with ethyl acetate (3×5 mL). The combined organic layers were dried over anhydrous sodium sulfate, concentrated to afford the title compound (23.0 mg, 89% yield) as yellow oil; LCMS (ESI, M+1): m/z=195.2.

Step C. 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-N-isopropyl-5,6,7,8-tetrahydro-4H-pyrazolo[1,5-a][1,4]diazepin-2-amine: To a mixture of tert-butyl 7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (50.0 mg, 1.0 equiv) and N-isopropyl-5,6,7,8-tetrahydro-4H-pyrazolo[1,5-a][1,4]diazepin-2-amine(21.0 mg, 1.5 equiv) in DMF (0.5 mL) was added DIEA (27.9 mg, 3.0 equiv). The reaction was stirred at 40° C. for 12 hours. The mixture was filtered and purified by reversed phase flash chromatography [C18, 0.1% formic acid condition] to afford the title compound (40.0 mg, 73% yield) as yellow oil; LCMS (ESI, M+1): m/z=717.5.

Step D. N-(5-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-isopropylacetamide: To a mixture of 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N-isopropyl-5,6,7,8-tetrahydro-4H-pyrazolo[1,5-a][1,4]diazepin-2-amine (60 mg, 1.0 equiv) in THE (2 mL) were added DMF (285 mg, 46 equiv) and NaH (5.02 mg, 60% purity, 1.5 equiv) at 0° C. The reaction was stirred at 20° C. for 30 minutes. To the resulting mixture was added Ac20 (17.1 mg, 2.0 equiv) in THF (1 mL) at 0° C. The reaction was stirred at 20° C. for 2 hours. The mixture was diluted with saturated NaHCO₃ aqueous solution (5 mL) and extracted with EtOAc (3×10 mL). The combined organic layers were washed with brine, dried over anhydrous sodium sulfate, concentrated, and purified by reversed phase flash chromatography [C18, 0.1% formic acid condition] to afford the title compound (50 mg, 77% yield) as white solid; 1H NMR (400 MHz, chloroform-d) δ=7.60-7.55 (m, 1H), 7.23-7.17 (m, 2H), 7.03-7.00 (m, 1H), 5.96-5.92 (m, 1H), 5.35-5.14 (m, 3H), 4.93-4.80 (m, 2H), 4.65-4.55 (m, 1H), 4.55-4.40 (m, 2H), 4.26-4.17 (m, 1H), 4.14-4.03 (m, 1H), 4.02-3.93 (m, 2H), 3.91-3.81 (m, 1H), 3.80-3.71 (m, 1H), 3.56-3.51 (m, 3H), 3.49-3.43 (m, 1H), 3.42-3.31 (m, 2H), 3.30-3.19 (m, 2H), 3.18-3.05 (m, 3H), 3.02-2.92 (m, 1H), 2.65-2.55 (m, 1H), 2.38-2.22 (m, 2H), 2.20-2.05 (m, 3H), 2.00-1.84 (m, 3H), 1.83-1.80 (m, 3H), 1.15-1.10 (m, 3H), 1.08-1.02 (m, 6H); 19F NMR (400 MHz, chloroform-d) 6 =−119.097, -173.140; LCMS (ESI, M+1): m/z=759.1.

Step E. N-(5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-isopropylacetamide: To a mixture of N-(5-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-isopropylacetamide (45 mg, 1.0 equiv) in ACN (112 μL) was added HCl•MeOH (4 M, 0.2 mL, 15 equiv). The reaction was stirred at 20° C. for 1 hour. The mixture was concentrated, dissolved in MeOH (1 mL), neutralized with solid NaHCO₃, filtered, and purified by prep-HPLC [Phenomenex luna C18 150×25 mm×10 μm; A: water (FA), B: ACN; B %: 21%-51% over 10 min] to afford the title compound (42 mg, 96% yield, 0.3 equiv FA) as yellow solid. 1H NMR (400 MHz, dimethylsulfoxide-d6) δ=10.75-8.99 (m, 1H), 7.63-7.56 (m, 1H), 7.29-7.19 (m, 1H), 7.02-6.95 (m, 2H), 6.08-6.02 (m, 1H), 5.33-5.12 (m, 1H), 4.96-4.84 (m, 1H), 4.80-4.70 (m, 1H), 4.68-4.58 (m, 1H), 4.52-4.38 (m, 2H), 4.09-3.97 (m, 1H), 3.96-3.89 (m, 1H), 3.88-3.77 (m, 3H), 3.59 (br d, J=16.0 Hz, 2H), 3.48-3.38 (m, 2H), 3.18-3.07 (m, 2H), 3.07-3.01 (m, 2H), 3.00-2.91 (m, 1H), 2.84-2.75 (m, 1H), 2.65-2.58 (m, 1H), 2.29-2.15 (m, 1H), 2.07-2.01 (m, 1H), 2.00-1.86 (m, 3H), 1.85-1.76 (m, 1H), 1.76-1.66 (m, 2H), 1.65-1.52 (m, 3H), 1.10-1.01 (m, 3H), 1.00-0.84 (m, 6H); 19F NMR (400 MHz, dimethylsulfoxide-d6) δ=−121.409, -172.099; LCMS (ESI, M+1): m/z=715.5.

### Example 288

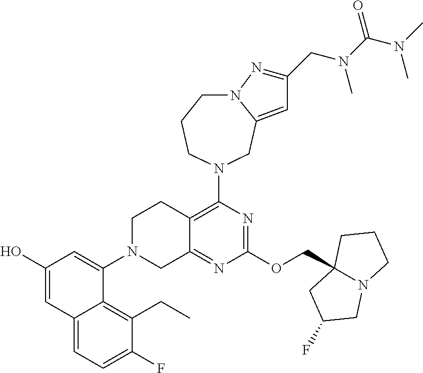 [SMILES: CCc1c(F)ccc2cc(O)cc(N3CCc4c(nc(OC[C@@]56CCCN5C[C@H](F)C6)nc4N4CCCn5nc(CN(C)C(=O)N(C)C)cc5C4)C3)c12]

### 1-((5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methyl)-1,3,3-trimethylurea

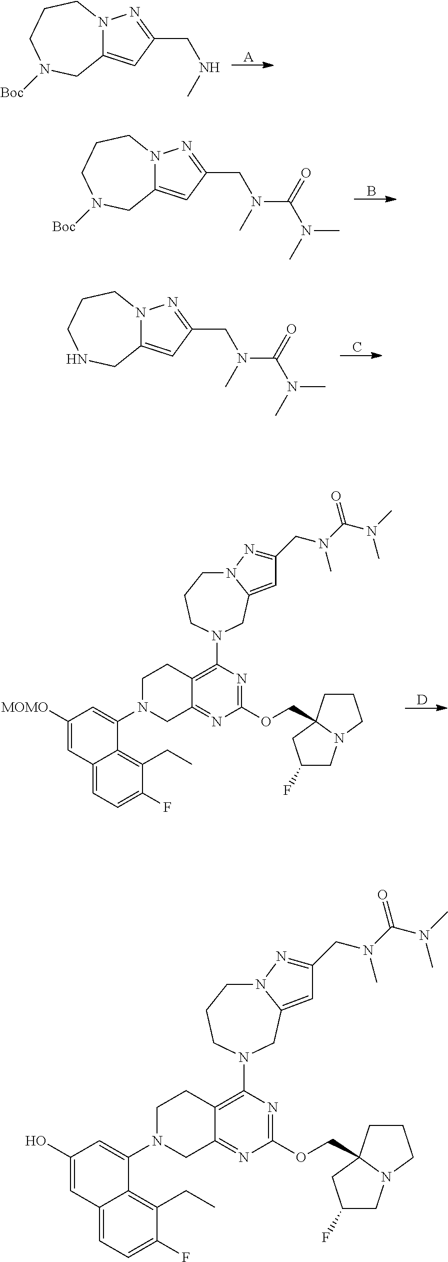

Step A. tert-butyl 2-((1,3,3-trimethylureido)methyl)-7,8-dihydro-4H-pyrazolo[1,5-a][1,4]diazepine-5(6H)-carboxylate: To a mixture of tert-butyl 2-((methylamino)methyl)-7,8-dihydro-4H-pyrazolo[1,5-a][1,4]diazepine-5(6H)-carboxylate (80.0 mg, 1.0 equiv) and N,N-diethylpropan-2-amine (742 mg, 20 equiv) in DCM (0.5 mL) was added dimethylcarbamic chloride (61.4 mg, 2.0 equiv). The reaction was stirred at 25° C. for 2 hours. The mixture was concentrated and purified by reversed phase flash chromatography [water (FA, 0.1%)/acetonitrile] to afford the title compound (40 mg, 39% yield) as yellow oil. LCMS (ESI, M+1): m/z=352.1.

Step B. 1,1,3-trimethyl-3-((5,6,7,8-tetrahydro-4H-pyrazolo[1,5-a][1,4]diazepin-2-yl)methyl)urea: A mixture of tert-butyl 2-((1,3,3-trimethylureido)methyl)-7,8-dihydro-4H-pyrazolo[1,5-a][1,4]diazepine-5(6H)-carboxylate (30.0 mg, 1.0 equiv) in HCl/dioxane (4 M, 1.0 mL, 47 equiv) was stirred at 20° C. for 0.5 hours. The mixture was concentrated under vacuum to afford the title compound (24.0 mg) as white oil and used into next step without further purification.

The last two steps were performed according to Example 248. The title compound was obtained as yellow solid (FA salt). 1H NMR (400 MHz, METHANOL-d4) δ=7.52 (dd, J=5.6, 8.8 Hz, 1H), 7.15 (t, J=9.6 Hz, 1H), 6.97 (s, 2H), 6.17 (s, 1H), 5.44 (s, 1H), 5.10-4.96 (m, 1H), 4.76 (br dd, J=3.6, 16.4 Hz, 2H), 4.51-4.37 (m, 2H), 4.34-4.15 (m, 4H), 4.13-4.00 (m, 2H), 3.98-3.84 (m, 1H), 3.69 (br d, J=17.6 Hz, 1H), 3.57-3.47 (m, 1H), 3.46-3.34 (m, 3H), 3.27-3.14 (m, 3H), 3.12-3.01 (m, 1H), 2.93-2.53 (m, 10H), 2.41-2.18 (m, 3H), 2.17-1.97 (m, 4H), 1.95-1.83 (m, 1H), 1.18-1.01 (m, 3H). LCMS (ESI, M+1): m/z=730.3.

### Example 289

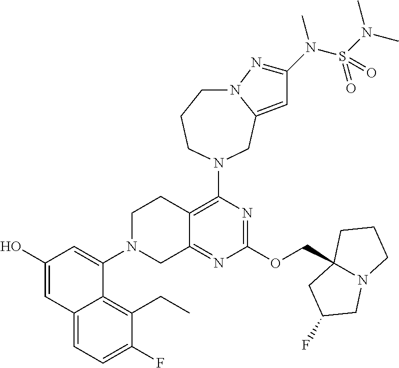 [SMILES: CCc1c(F)ccc2cc(O)cc(N3CCc4c(nc(OC[C@@]56CCCN5C[C@H](F)C6)nc4N4CCCn5nc(N(C)S(=O)(=O)N(C)C)cc5C4)C3)c12]

### 7-(8-ethyl-7-fluoro-3-hydroxy-1-naphthyl)-2-(((2R,7aS)-2-fluorotetrahydro-1H-pyrrolizin-7a(5H)-yl)methoxy)-4-(2-((dimethylsulfamoyl)methylamino)-7,8-dihydro-4H-pyrazolo[1,5-a][1,4]diazepin-5(6H)-yl)-6,8-dihydro-5H-pyrido[3,4-d]pyrimidine

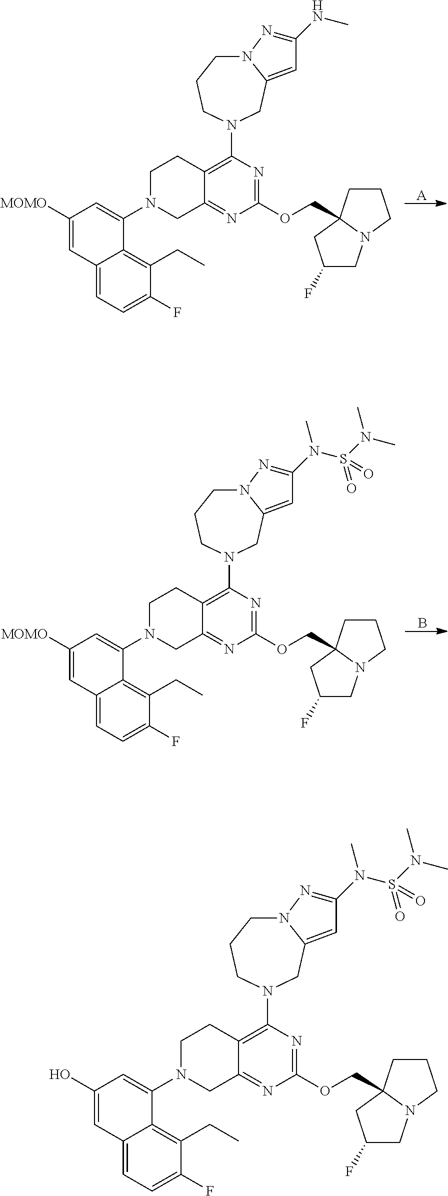

Step A. 7-(8-ethyl-7-fluoro-3-(methoxymethoxy)-1-naphth1)-2-(((2R,7aS)-2-fluorotetrahydro-1H-pyrrolizin-7a(5H)-yl)methoxy)-4-(2-((dimethylsulfamoyl)methylamino)-7,8-dihydro-4H-pyrazolo[1,5-a][1,4]diazepin-5(6H)-yl)-6,8-dihydro-5H-pyrido[3,4-d] pyrimidine: To a mixture of 5-(7-(8-ethyl-7-fluoro-3-(methoxymethoxy)naphthalen-1-yl)-2-(((2R,7aS)-2-fluorotetrahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepin-2-amine (200 mg, 1.0 equiv) in pyridine (490 mg, 21 equiv) were added dimethylsulfamoyl chloride (104 mg, 2.5 equiv) and THF (0.50 mL). The reaction was stirred at 20° C. for 2 hours. The mixture was concentrated and purified by reversed phase flash chromatography [C18, 0.1% formic acid condition] to afford the title compound (77.0 mg, 31% yield) as yellow solid; LCMS (ESI, M+1): m/z=796.5.

Step B. 7-(8-ethyl-7-fluoro-3-hydroxy-1-naphthyl)-2-(((2R,7aS)-2-fluorotetrahydro-1H-pyrrolizin-7a(5H)-yl)methoxy)-4-(2-((dimethylsulfamoyl)methylamino)-7,8-dihydro-4H-pyrazolo[1,5-a][1,4]diazepin-5(6H)-yl)-6,8-dihydro-5H-pyrido[3,4-d]pyrimidine: To a mixture of 7-(8-ethyl-7-fluoro-3-(methoxymethoxy)-1-naphthyl)-2-(((2R,7aS)-2-fluorotetrahydro-1H-pyrrolizin-7a(5H)-yl)methoxy)-4-(2-((dimethylsulfamoyl)methylamino)-7,8-dihydro-4H-pyrazolo[1,5-a][1,4]diazepin-5(6H)-yl)-6,8-dihydro-5H-pyrido[3,4-d]pyrimidine (77.0 mg, 1.0 equiv) in ACN (0.50 mL) was added HCl-MeOH (4 M, 1.0 mL, 41 equiv). The reaction was stirred at 20° C. for 0.5 hours. The mixture was concentrated, dissolved in methanol (3.0 mL), neutralized with solid NaHCO₃, filtered, purified by prep-HPLC [Phenomenex Luna C18 250×50 mm×15 μm; A: water (FA), B: ACN; B %: 22%-52% over 15 min] and lyophilized to afford the title compound (72.0 mg, 98% yield, FA salt) as yellow solid. 1H NMR (400 MHz, dimethylsulfoxide-d6+deuterium oxide-d2) δ=7.58 (dd, J=6.0, 8.8 Hz, 1H), 7.23 (t, J=9.2 Hz, 1H), 6.99 (s, 2H), 6.13 (s, 1H), 5.35-5.14 (m, 1H), 4.90 (br d, J=16.4 Hz, 1H), 4.69 (br d, J=16.4 Hz, 1H), 4.36 (br s, 2H), 4.10 (br d, J=12.8 Hz, 1H), 4.00-3.74 (m, 4H), 3.59 (br s, 1H), 3.41 (br d, J=8.0 Hz, 1H), 3.34-3.22 (m, 2H), 3.15 (s, 4H), 3.13-3.07 (m, 3H), 3.03 (br s, 1H), 2.87-2.78 (m, 1H), 2.66 (s, 7H), 2.22-2.10 (m, 1H), 2.09-1.97 (m, 2H), 1.96-1.86 (m, 2H), 1.84-1.66 (m, 3H), 1.05 (br t, J=7.2 Hz, 3H); 19F NMR (400 MHz, dimethylsulfoxide-d6+deuterium oxide-d2) δ=−121.273, -171.969; LCMS (ESI, M+1): m/z=752.5.

### Example 290

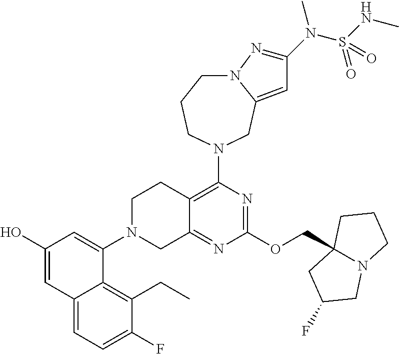 [SMILES: CCc1c(F)ccc2cc(O)cc(N3CCc4c(nc(OC[C@@]56CCCN5C[C@H](F)C6)nc4N4CCCn5nc(N(C)S(=O)(=O)NC)cc5C4)C3)c12]

### 7-(8-ethyl-7-fluoro-3-hydroxy-1-naphthyl)-2-(((2R,7aS)-2-fluorotetrahydro-1H-pyrrolizin-7a(5H)-yl)methoxy)-4-(2-((methylsulfamoyl)methylamino)-7,8-dihydro-4H-pyrazolo[1,5-a][1,4]diazepin-5(6H)-yl)-6,8-dihydro-5H-pyrido[3,4-d]pyrimidine

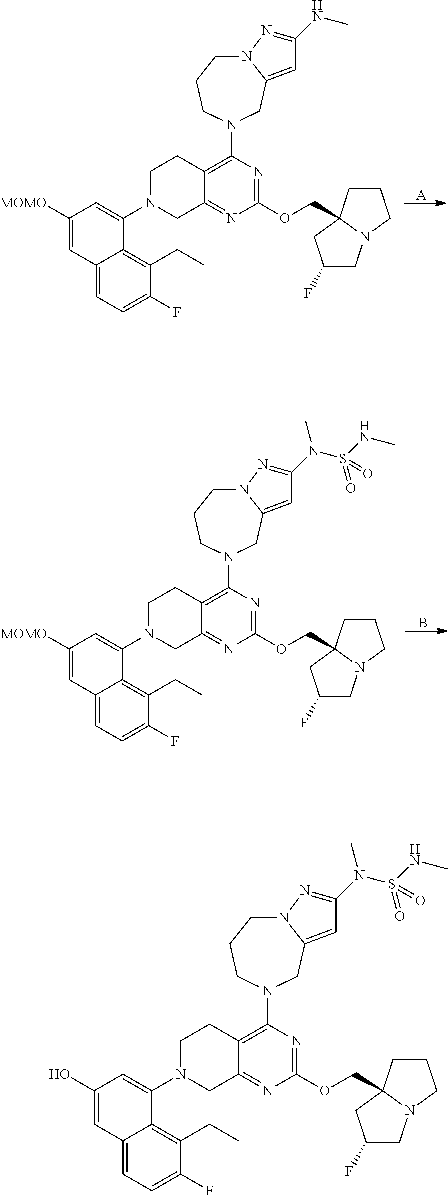

Step A. 7-(8-ethyl-7-fluoro-3-(methoxymethoxy)-1-naphthyl)-2-(((2R,7aS)-2-fluorotetrahydro-1H-pyrrolizin-7a(5H)-yl)methoxy)-4-(2-((methylsulfamoyl)methylamino)-7,8-dihydro-4H-pyrazolo[1,5-a][1,4]diazepin-5(6H)-yl)-6,8-dihydro-5H-pyrido[3,4-d]pyrimidine: To a mixture of 5-(7-(8-ethyl-7-fluoro-3-(methoxymethoxy)naphthalen-1-yl)-2-(((2R,7aS)-2-fluorotetrahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepin-2-amine (120 mg, 1.0 equiv) in pyridine (490 mg, 35 equiv) were added methylsulfamoyl chloride (56.4 mg, 2.5 equiv) and THF (0.50 mL). The reaction was stirred at 20° C. for 2 hours. The mixture was concentrated and purified by reversed phase flash chromatography [C18, 0.1% formic acid condition] to afford the title compound (52.0 mg, 38% yield) as yellow solid; LCMS (ESI, M+1): m/z=782.4.

Step B. 7-(8-ethyl-7-fluoro-3-hydroxy-1-naphthyl)-2-(((2R,7aS)-2-fluorotetrahydro-1H-pyrrolizin-7a(5H)-yl)methoxy)-4-(2-((methylsulfamoyl)methylamino)-7,8-dihydro-4H-pyrazolo[1,5-a][1,4]diazepin-5(6H)-yl)-6,8-dihydro-5H-pyrido[3,4-d]pyrimidine: To a mixture of 7-(8-ethyl-7-fluoro-3-(methoxymethoxy)-1-naphthyl)-2-(((2R,7aS)-2-fluorotetrahydro-1H-pyrrolizin-7a(5H)-yl)methoxy)-4-(2-((methylsulfamoyl)methylamino)-7,8-dihydro-4H-pyrazolo[1,5-a][1,4]diazepin-5(6H)-yl)-6,8-dihydro-5H-pyrido[3,4-d]pyrimidine (52.0 mg, 1.0 equiv) in ACN (0.50 mL) was added HCl-MeOH (4M, 1.0 mL, 60 equiv). The reaction was stirred at 20° C. for 0.5 hours. The mixture was concentrated, dissolved in methanol (3.0 mL), neutralized with solid NaHCO₃, filtered, purified by prep-HPLC [Phenomenex Luna C18 150×25 mm×10 μm; A: water (FA), B: ACN; B %: 23%-53% over 58 min] and lyophilized to afford the title compound (12.2 mg, 24% yield, FA salt) as off-white solid. 1H NMR (400 MHz, dimethylsulfoxide-d6) δ=9.84-9.58 (m, 1H), 7.59 (dd, J=6.0, 8.8 Hz, 1H), 7.52-7.37 (m, 1H), 7.24 (t, J=9.2 Hz, 1H), 6.99 (s, 2H), 6.15 (s, 1H), 5.35-5.14 (m, 1H), 4.98-4.85 (m, 1H), 4.68 (br d, J=16.0 Hz, 1H), 4.42-4.29 (m, 2H), 4.10 (br dd, J=2.4, 12.0 Hz, 1H), 3.96-3.76 (m, 4H), 3.59 (br d, J=17.6 Hz, 1H), 3.42 (br d, J=7.2 Hz, 2H), 3.16-3.01 (m, 7H), 2.98 (s, 1H), 2.87-2.75 (m, 1H), 2.70-2.62 (m, 1H), 2.45 (d, J=4.8 Hz, 3H), 2.15 (br dd, J=6.8, 7.6 Hz, 1H), 2.06 (br d, J=7.6 Hz, 1H), 2.04-1.91 (m, 3H), 1.77 (br s, 2H), 1.76-1.65 (m, 2H), 1.06 (t, J=7.2 Hz, 3H); 19F NMR (400 MHz, dimethylsulfoxide-d6) δ=−121.371, -171.962; LCMS (ESI, M+1): m/z=738.4.

### Example 291

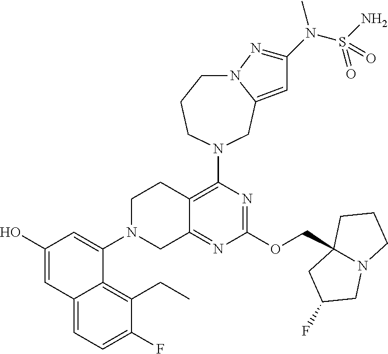 [SMILES: CCc1c(F)ccc2cc(O)cc(N3CCc4c(nc(OC[C@@]56CCCN5C[C@H](F)C6)nc4N4CCCn5nc(N(C)S(N)(=O)=O)cc5C4)C3)c12]

### 7-(8-ethyl-7-fluoro-3-hydroxy-1-naphthyl)-2-(((2R,7aS)-2-fluorotetrahydro-1H-pyrrolizin-7a(5H)-yl)methoxy)-4-(2-((sulfamoyl)methylamino)-7,8-dihydro-4H-pyrazolo[1,5-a][1,4]diazepin-5(6H)-yl)-6,8-dihydro-5H-pyrido[3,4-d]pyrimidine

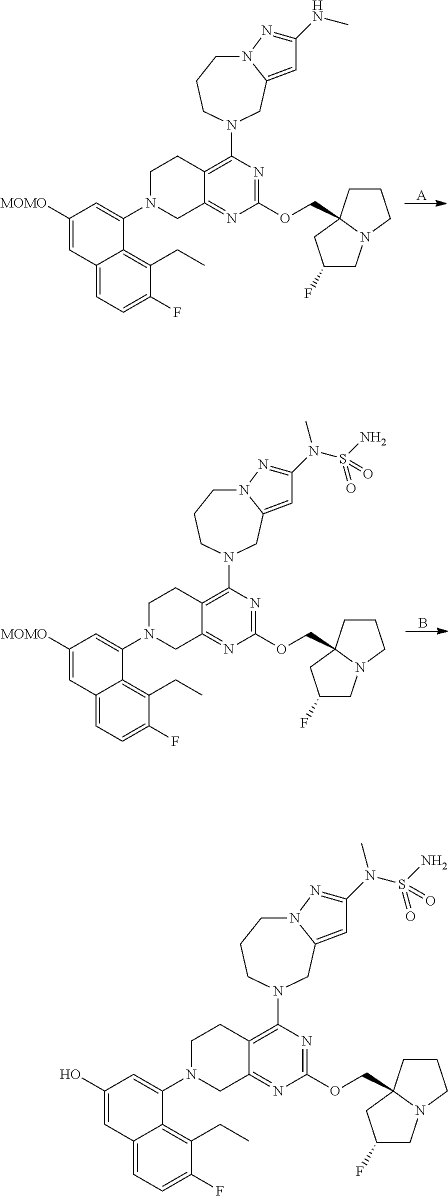

Step A. 7-(8-ethyl-7-fluoro-3-(methoxymethoxy)-1-naphthyl)-2-(((2R,7aS)-2-fluorotetrahydro-1H-pyrrolizin-7a(5H)-yl)methoxy)-4-(2-((sulfamoyl)methylamino)-7,8-dihydro-4H-pyrazolo[1,5-a][1,4]diazepin-5(6H)-yl)-6,8-dihydro-5H-pyrido[3,4-d]pyrimidine: To a mixture of 5-(7-(8-ethyl-7-fluoro-3-(methoxymethoxy)naphthalen-1-yl)-2-(((2R,7aS)-2-fluorotetrahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepin-2-amine (120 mg, 1.0 equiv) in pyridine (490 mg, 35 equiv) was added sulfamoyl chloride (50.3 mg, 2.5 equiv) in THF (0.50 mL). The reaction was stirred at 20° C. for 2 hours. The mixture was concentrated and purified by reversed phase flash chromatography [C18, 0.1% formic acid condition] to afford the title compound (46.0 mg, 34% yield) as yellow solid; LCMS (ESI, M+1): m/z=768.4.

Step B. 7-(8-ethyl-7-fluoro-3-hydroxy-1-naphthyl)-2-(((2R,7aS)-2-fluorotetrahydro-1H-pyrrolizin-7a(5H)-yl)methoxy)-4-(2-((sulfamoyl)methylamino)-7,8-dihydro-4H-pyrazolo[1,5-a][1,4]diazepin-5(6H)-yl)-6,8-dihydro-5H-pyrido[3,4-d]pyrimidine: To a mixture of 7-(8-ethyl-7-fluoro-3-(methoxymethoxy)-1-naphthyl)-2-(((2R,7aS)-2-fluorotetrahydro-1H-pyrrolizin-7a(5H)-yl)methoxy)-4-(2-((sulfamoyl)methylamino)-7,8-dihydro-4H-pyrazolo[1,5-a][1,4]diazepin-5(6H)-yl)-6,8-dihydro-5H-pyrido[3,4-d]pyrimidine (46.0 mg, 1.0 equiv) in ACN (0.50 mL) was added HCl•MeOH (1.0 mL, 66 equiv). The reaction was stirred at 20° C. for 0.5 hours. The mixture was concentrated, dissolved in methanol (3.0 mL), neutralized with solid NaHCO₃, filtered, purified by prep-HPLC [Phenomenex Luna C18 150 x 25 mm×10 μm; A: water (FA), B: ACN; B %: 20%-50% over 58 min] and lyophilized to afford the title compound (12.4 mg, 27% yield, FA salt) as orange solid. 1H NMR (400 MHz, dimethylsulfoxide-d6+deuterium oxide-d2) δ=7.57 (dd, J=6.0, 8.8 Hz, 1H), 7.23 (br t, J=9.2 Hz, 1H), 6.98 (s, 2H), 6.17 (s, 1H), 5.41-5.17 (m, 1H), 4.94-4.83 (m, 1H), 4.68 (br d, J=16.0 Hz, 1H), 4.32 (br s, 2H), 4.14-4.06 (m, 1H), 4.06-3.94 (m, 2H), 3.93-3.77 (m, 3H), 3.42 (br d, J=9.2 Hz, 1H), 3.27-3.10 (m, 6H), 3.06 (s, 3H), 2.91-2.83 (m, 1H), 2.66 (br d, J=4.4 Hz, 1H), 2.13 (br d, J=4.0 Hz, 2H), 2.06 (br s, 1H), 2.02-1.96 (m, 1H), 1.95-1.71 (m, 5H), 1.04 (br t, J=7.2 Hz, 3H); 19F NMR (400 MHz, dimethylsulfoxide-d6+deuterium oxide-d2) δ=−121.153, -171.917; LCMS (ESI, M+1): m/z=724.6.

### Example 292

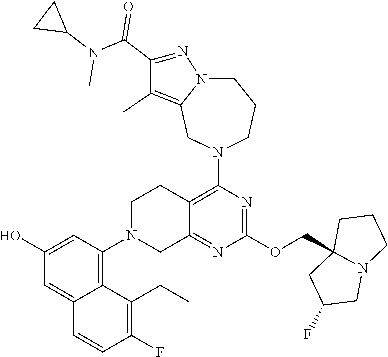 [SMILES: CCc1c(F)ccc2cc(O)cc(N3CCc4c(nc(OC[C@@]56CCCN5C[C@H](F)C6)nc4N4CCCn5nc(C(=O)N(C)C6CC6)c(C)c5C4)C3)c12]

### N-cyclopropyl-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,3-dimethyl-5,6,7,8-tetrahydro-4H-pyrazolo[1,5-a][1,4]diazepine-2-carboxamide

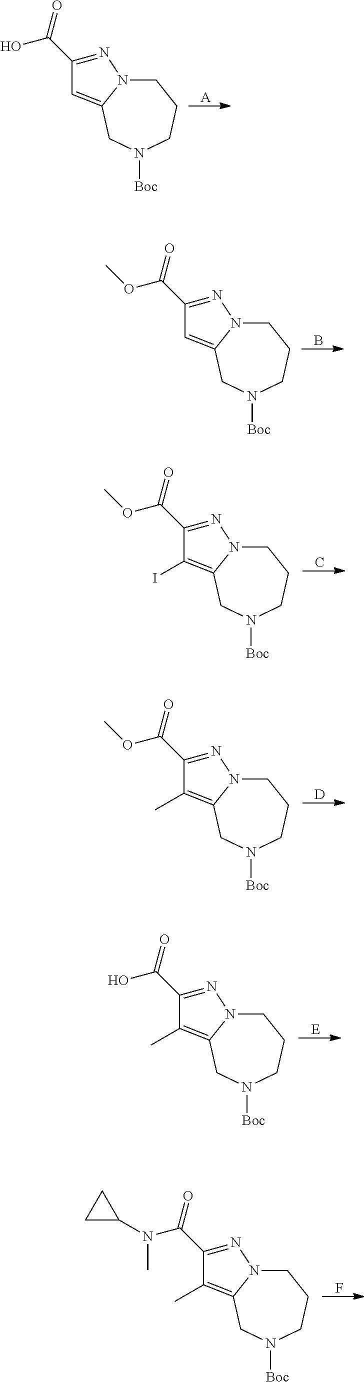

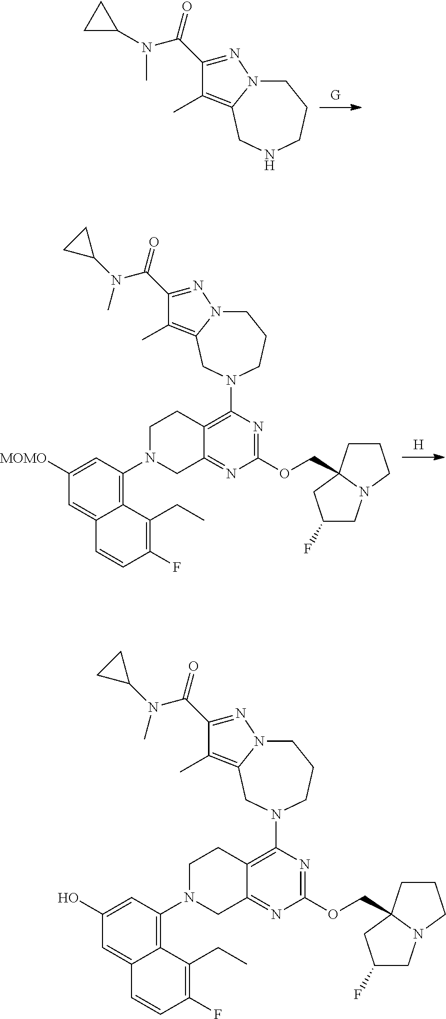

Step A. 5-tert-butyl 2-methyl 7,8-dihydro-4H-pyrazolo[1,5-a][1,4]diazepine-2,5(6H)-dicarboxylate: To a solution of 5-(tert-butoxycarbonyl)-5,6,7,8-tetrahydro-4H-pyrazolo[1,5-a][1,4]diazepine-2-carboxylic acid (5.0 g, 1.0 equiv) in MeOH (25 mL) was added diazomethyl(trimethyl)silane (2 M, 18 mL, 2.0 equiv). The mixture was stirred at 25° C. for 0.5 hours. The reaction was filtered and concentrated to afford the title compound (2.40 g, 81% yield) as yellow solid. LCMS (ESI, M+1): m/z=296.2.

Step B. 5-tert-butyl 2-methyl 3-iodo-7,8-dihydro-4H-pyrazolo[1,5-a][1,4]diazepine-2,5(6H)-dicarboxylate: To a solution of 5-tert-butyl 2-methyl 7,8-dihydro-4H-pyrazolo[1,5-a][1,4]diazepine-2,5(6H)-dicarboxylate (2.40 g, 1.0 equiv) in CH3COOH (25 mL) was added NIS (3.70 g, 2.0 equiv). The mixture was stirred at 80° C. for 0.5 hours. The reaction was quenched with saturated NaHCO₃ solution (80 mL) at 0° C. The filtrate was extracted with EtOAc (3×100 mL). The combined organic layers were washed with brine (3×50 mL), dried over anhydrous sodium sulfate, concentrated, and purified by reversed phase flash chromatography [C18, 0.1% formic acid condition] to afford the title compound (2.05 g, 59% yield) as yellow solid; LCMS (ESI, M+1): m/z=422.1.

Step C. 5-tert-butyl 2-methyl 3-methyl-7,8-dihydro-4H-pyrazolo[1,5-a][1,4]diazepine-2,5(6H)-dicarboxylate: A mixture of 5-tert-butyl 2-methyl 3-iodo-7,8-dihydro-4H-pyrazolo[1,5-a][1,4]diazepine-2,5(6H)-dicarboxylate (2.0 g, 1.0 equiv), 2,4,6-trimethyl-1,3,5,2,4,6-trioxatriborinane (4.80 g, 50% purity, 4.0 equiv), Pd(dppf)C12 (347 mg, 0.1 equiv), and K2CO3 (2.0 g, 3.0 equiv) in DMF (20 mL) was degassed and purged with N2 3 times. The mixture was stirred at 100° C. for 5 hours under N2 atmosphere. The reaction was filtered, concentrated, and purified by reversed phase flash chromatography [C18, 0.1% formic acid condition] to afford the title compound (1.20 g, 82% yield) as white solid; LCMS (ESI, M+1): m/z=310.2.

Step D. 5-(tert-butoxycarbonyl)-3-methyl-5,6,7,8-tetrahydro-4H-pyrazolo[1,5-a][1,4]diazepine-2-carboxylic acid: To a solution of 5-tert-butyl 2-methyl 3-methyl-7,8-dihydro-4H-pyrazolo[1,5-a][1,4]diazepine-2,5(6H)-dicarboxylate (3.0 g, 1.0 equiv) in THF (15 mL) were added LiOH H₂O (1.20 g, 3.0 equiv), MeOH (7.5 mL) and H₂O (15 mL). The mixture was stirred at 25° C. for 1 hour. The reaction was concentrated. The residue was adjusted to pH 8 by HCl (2.5 ml, 2M). The residue was extracted with EtOAc (3×100 mL). The combined organic layers were dried over anhydrous sodium sulfate, concentrated to afford the title compound (2.50 g, crude) as white solid; LCMS (ESI, M+1): m/z=296.2.

Step E. tert-butyl 2-(cyclopropyl(methyl)carbamoyl)-3-methyl-7,8-dihydro-4H-pyrazolo[1,5-a][1,4]diazepine-5(6H)-carboxylate: To a solution of 5-(tert-butoxycarbonyl)-3-methyl-5,6,7,8-tetrahydro-4H-pyrazolo[1,5-a][1,4]diazepine-2-carboxylic acid (1.0 g, 1.0 equiv) in DMF (10 mL) were added HATU (2.0 g, 1.5 equiv), N-methylcyclopropanamine (1.20 g, 3.0 equiv, HCl) and N,N-diethylpropan-2-amine (3.50 g, 8.0 equiv). The mixture was stirred at 25° C. for 12 hours. The reaction was filtered. The filtrate was purified with reversed phase flash chromatography [C18, 0.1% formic acid condition] to afford the title compound (1.10 g, 93% yield) as white solid; LCMS (ESI, M+1): m/z=349.2.

Step F. N-cyclopropyl-N,3-dimethyl-5,6,7,8-tetrahydro-4H-pyrazolo[1,5-a][1,4]diazepine-2-carboxamide: To a solution of tert-butyl 2-(cyclopropyl(methyl)carbamoyl)-3-methyl-7,8-dihydro-4H-pyrazolo[1,5-a][1,4]diazepine-5(6H)-carboxylate (1.10 g, 1.0 equiv) in ACN (12 mL) was added HCl•dioxane (4 M, 11 mL) at 0° C. The mixture was stirred at 25° C. for 1 hour. The reaction was concentrated. The residue was adjusted to pH 10 by saturated NaOH solution (10 mL) at 0° C. The reaction was extracted with DCM (3×100 mL). The combined organic layers were dried over anhydrous sodium sulfate and concentrated to afford the title compound (877 mg, crude) as yellow solid; LCMS (ESI, M+1): m/z=249.1.

The last two steps were performed according to Example 248. The title compound was obtained as yellow solid (FA salt). 1H NMR (400 MHz, METHANOL-d4) δ=8.51 (s, 1H), 7.51-7.48 (m, 1H), 7.13 (t, J=9.4 Hz, 1H), 6.96 (s, 2H), 5.46-5.30 (m, 1H), 4.75 (d, J=7.4 Hz, 1H), 4.53-4.36 (m, 2H), 4.25-3.94 (m, 5H), 3.68-3.63 (m, 1H), 3.55-3.36 (m, 6H), 3.26-3.12 (m, 3H), 3.11-3.03 (m, 3H), 2.89 (s, 1H), 2.68 (d, J=14.8 Hz, 1H), 2.51-2.21 (m, 4H), 2.21-1.85 (m, 8H), 1.08 (t, J=7.2 Hz, 3H), 0.88-0.34 (m, 4H); LCMS (ESI, M+1): m/z=727.5.

### Example 293

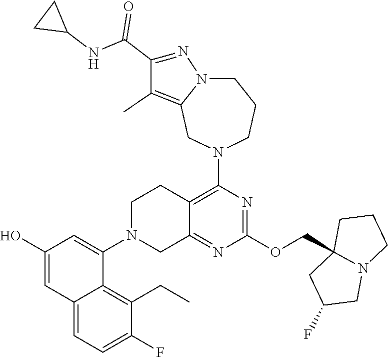 [SMILES: CCc1c(F)ccc2cc(O)cc(N3CCc4c(nc(OC[C@@]56CCCN5C[C@H](F)C6)nc4N4CCCn5nc(C(=O)NC6CC6)c(C)c5C4)C3)c12]

### N-cyclopropyl-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-3-methyl-5,6,7,8-tetrahydro-4H-pyrazolo[1,5-a][1,4]diazepine-2-carboxamide

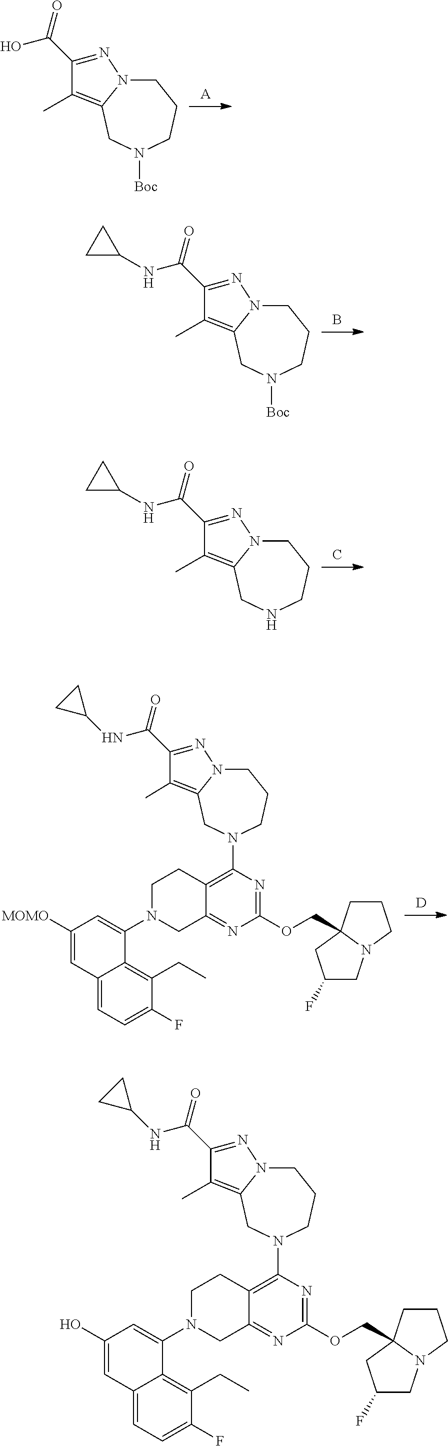

Step A. tert-butyl 2-(cyclopropylcarbamoyl)-3-methyl-7,8-dihydro-4H-pyrazolo[1,5-a][1,4]diazepine-5(6H)-carboxylate: To a solution of 5-(tert-butoxycarbonyl)-3-methyl-5,6,7,8-tetrahydro-4H-pyrazolo[1,5-a][1,4]diazepine-2-carboxylic acid (1.0 g, 1.0 equiv) in DMF (10 mL) were added HATU (1.9 g, 1.5 equiv), cyclopropanamine (0.966 g, 5.0 equiv) and N,N-diethylpropan-2-amine (1.31 g, 3.0 equiv). The mixture was stirred at 25° C. for 12 hours. The reaction was filtered. The filtrate was purified with reversed phase flash chromatography [C18, 0.1% formic acid condition] to afford the title compound (1.00 g, 87% yield) as white solid.

Step B. N-cyclopropyl-3-methyl-5,6,7,8-tetrahydro-4H-pyrazolo[1,5-a][1,4]diazepine-2-carboxamide: To a solution of tert-butyl 2-(cyclopropylcarbamoyl)-3-methyl-7,8-dihydro-4H-pyrazolo[1,5-a][1,4]diazepine-5(6H)-carboxylate (1.00 g, 1.0 equiv) in ACN (10 mL) was added HCl•dioxane (4 M, 10 mL) at 0° C. The mixture was stirred at 25° C. for 1 hour. The reaction was concentrated. The residue was adjusted to pH 10 by saturated NaOH solution (10 mL) at 0° C. The reaction was extracted with DCM (3×100 mL). The combined organic layers were dried over anhydrous sodium sulfate and concentrated to afford the title compound (650 mg, crude) as yellow solid.

The last two steps were performed according to Example 248. The title compound was obtained as white solid (FA salt). 1H NMR (400 MHz, METHANOL-d4) δ=8.53-8.49 (m, 1H), 7.54-7.47 (m, 1H), 7.18-7.10 (m, 1H), 6.99-6.90 (m, 2H), 5.46-5.24 (m, 1H), 4.87-4.81 (m, 1H), 4.75-4.64 (m, 1H), 4.53-4.43 (m, 1H), 4.43-4.33 (m, 1H), 4.19-4.02 (m, 4H), 4.00-3.90 (m, 1H), 3.69-3.59 (m, 1H), 3.36 (s, 6H), 3.27-3.09 (m, 3H), 2.81-2.65 (m, 2H), 2.45-2.33 (m, 1H), 2.32 (s, 3H), 2.23 (s, 2H), 2.16-2.01 (m, 4H), 1.96-1.86 (m, 1H), 1.14-1.05 (m, 3H), 0.82-0.73 (m, 2H), 0.62-0.54 (m, 2H); LCMS (ESI, M+1): m/z=713.2.

### Example 294

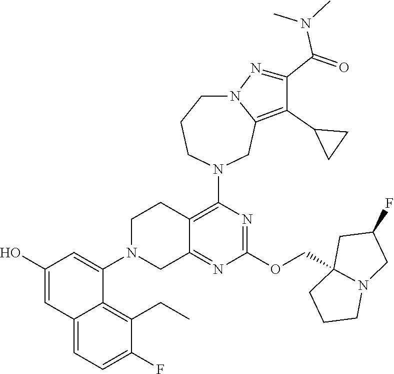 [SMILES: CCc1c(F)ccc2cc(O)cc(N3CCc4c(nc(OC[C@@]56CCCN5C[C@H](F)C6)nc4N4CCCn5nc(C(=O)N(C)C)c(C6CC6)c5C4)C3)c12]

### 3-cyclopropyl-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide

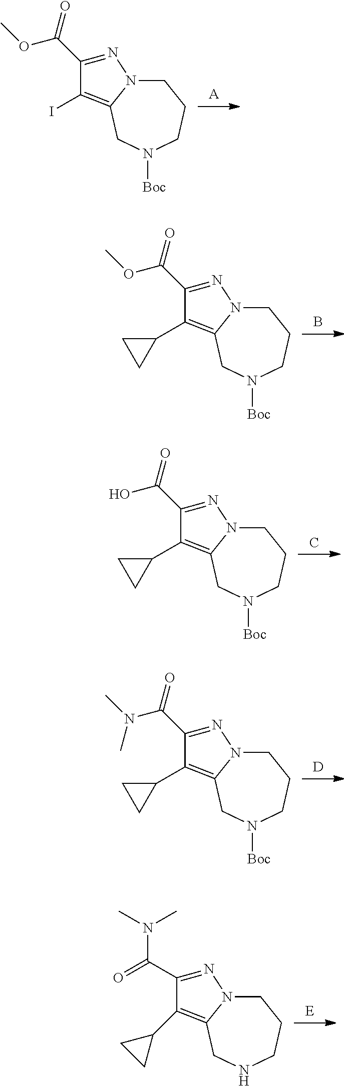

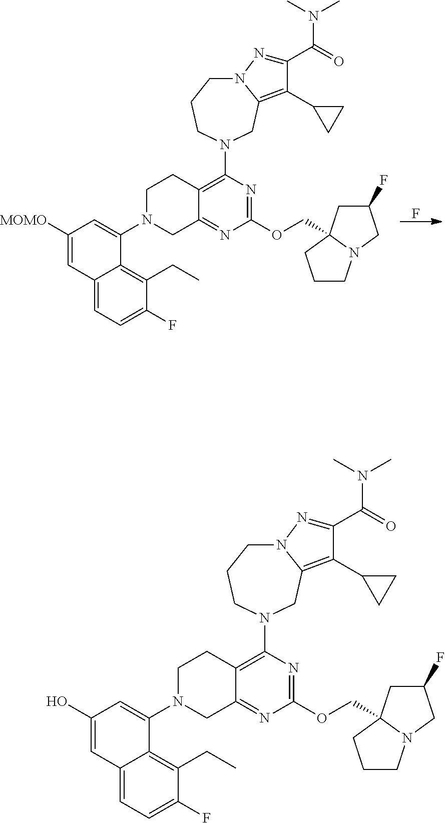

Step A. 5-tert-butyl 2-methyl 3-cyclopropyl-7,8-dihydro-4H-pyrazolo[1,5-a][1,4]diazepine-2,5(6H)-dicarboxylate: To a solution of 5-tert-butyl 2-methyl 3-iodo-7,8-dihydro-4H-pyrazolo[1,5-a][1,4]diazepine-2,5(6H)-dicarboxylate (3.0 g, 1 equiv) in DMF (48 mL) were added cyclopropylboronic acid (1.22 g, 2 equiv) and K2CO₃ (2.95 g, 3 equiv) and was then degassed and purged with N2 atmosphere. To the reaction was added Pd(dppf)C12 (521 mg, 0.1 equiv) under N2. The reaction was stirred at 100° C. for 4 hours. The reaction was filtered by Celite, diluted with water (500 mL) and extracted with ethyl acetate (4×150 mL). The organic phase was dried over Na₂SO₄, concentrated, and purified by column chromatography (SiO₂, petroleum ether/ethyl acetate=30/1 to 1/2) It was then concentrated and purified by reversed phase chromatography [0.1% FA condition] to afford the title compound (1.9 g, 79% yield) as brown oil; LCMS (ESI, M+1): m/z=349.1.

Step B. 5-(tert-butoxycarbonyl)-3-cyclopropyl-5,6,7,8-tetrahydro-4H-pyrazolo[1,5-a][1,4]diazepine-2-carboxylic acid: To a solution of 5-tert-butyl 2-methyl 3-cyclopropyl-7,8-dihydro-4H-pyrazolo[1,5-a][1,4]diazepine-2,5(6H)-dicarboxylate (1.8 g, 1.0 equiv) in MeOH (18 mL) was added NaOH (1 M, 10.7 mL, 2.0 equiv) at 0° C. The mixture was stirred at 25° C. for 1 hour. The pH of the mixture was adjusted to 3 with 1M HCl aqueous at 0° C. The mixture was diluted with water (30 mL) and extracted with ethyl acetate (3×20 mL). The organic phase was dried over Na₂SO₄ and concentrated to afford the title compound (1.6 g) as brown solid; LCMS (ESI, M+1): m/z=322.1.

Step C. tert-butyl 3-cyclopropyl-2-(dimethylcarbamoyl)-7,8-dihydro-4H-pyrazolo[1,5-a][1,41diazepine-5(6H)-carboxylate: To a solution of 5-(tert-butoxycarbonyl)-3-cyclopropyl-5,6,7,8-tetrahydro-4H-pyrazolo[1,5-a][1,4]diazepine-2-carboxylic acid (1.4 g, 1.0 equiv) in DMF (5 mL) were added EDCI (1.67 g, 2.0 equiv), HOBt (706 mg, 1.2 equiv), TEA (4.4 g, 10 equiv) and N-methylmethanamine (2 M, 11 mL, 5.0 equiv). The mixture was stirred at 40° C. for 66 hours. The reaction was filtered and purified by reversed phase flash chromatography {0.1% FA condition] to afford the title compound (1.3 g, 88% yield) as brown oil; LCMS (ESI, M+1): m/z=349.1.

Step D. 3-cyclopropyl-N,N-dimethyl-5,6,7,8-tetrahydro-4H-pyrazolo[1,5-a][1,4]diazepine-2-carboxamide: To a solution of tert-butyl 3-cyclopropyl-2-(dimethylcarbamoyl)-7,8-dihydro-4H-pyrazolo[1,5-a][1,4]diazepine-5(6H)-carboxylate (200 mg, 1.0 equiv) in ACN (1 mL) was added HCl•dioxane (4 M, 3.00 mL, 21 equiv) slowly at 0° C. The reaction was stirred at 0° C. for 1 hour. The reaction was concentrated at 0° C. Then to the crude product was added MeOH (4 mL) and NaHCO₃slowly at 0° C. (pH=8) and was then filtered and concentrated. The residue was dissolved in DCM:MeOH=10:1 (5 mL) and stirred for 10 minutes. The mixture was filtered and the filter cake was washed with DCM:MeOH=10:1 (2×5 mL). The organic phase was concentrated to afford the title compound (120 mg, crude) as brown oil; LCMS (ESI, M+1): m/z=249.1.

The last two steps were performed according to Example 248. The title compound was obtained as white solid (FA salt). 1H NMR (400 MHz, METHANOL-d4) δ=8.53 (s, 1H), 7.51 (dd, J=5.8, 8.8 Hz, 1H), 7.15 (t, J=9.2 Hz, 1H), 6.93 (br d, J=2.8 Hz, 2H), 5.41-5.22 (m, 1H), 5.07 (br d, J=16.4 Hz, 1H), 4.82 (br d, J=16.4 Hz, 1H), 4.51-4.34 (m, 2H), 4.22-4.08 (m, 3H), 4.06-3.91 (m, 2H), 3.79-3.70 (m, 1H), 3.53-3.32 (m, 5H), 3.28 (br s, 1H), 3.24-3.13 (m, 2H), 3.13-3.03 (m, 4H), 3.00 (s, 3H), 2.78-2.66 (m, 1H), 2.38-2.09 (m, 5H), 2.08-1.83 (m, 3H), 1.68-1.58 (m, 1H), 1.08 (t, J=7.2 Hz, 3H), 0.86-0.59 (m, 2H), 0.53-0.20 (m, 2H); LCMS (ESI, M+1): m/z=727.5.

### Example 295

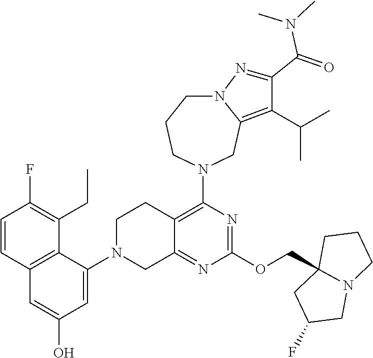 [SMILES: CCc1c(F)ccc2cc(O)cc(N3CCc4c(nc(OC[C@@]56CCCN5C[C@H](F)C6)nc4N4CCCn5nc(C(=O)N(C)C)c(C(C)C)c5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-3-isopropyl-N,N-dimethyl-5,6,7,8-tetrahydro-4H-pyrazolo[1,5-a][1,4]diazepine-2-carboxamide

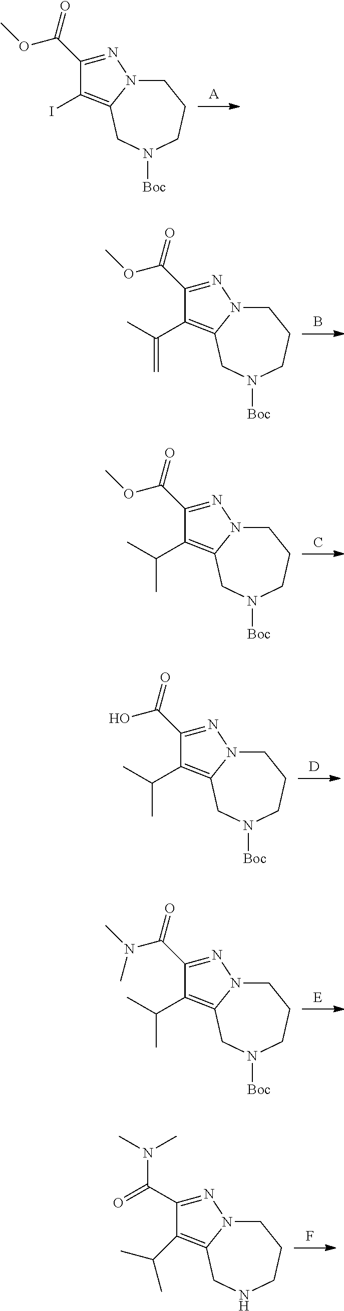

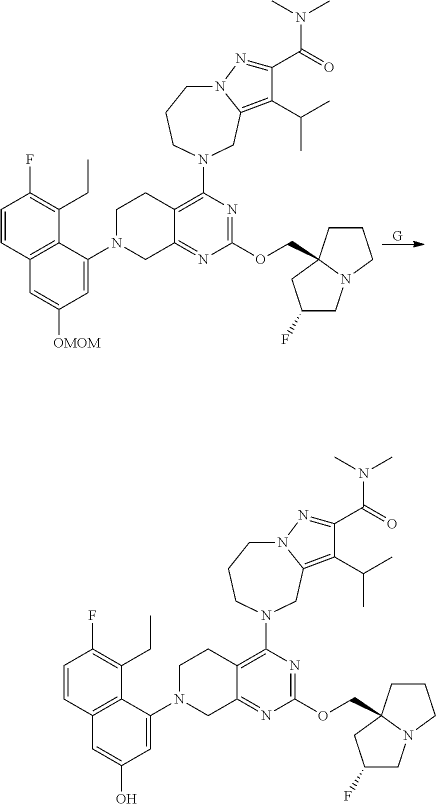

Step A. 5-tert-butyl 2-methyl 3-(prop-1-en-2-yl)-78-dihydro-4H-pyrazolo[1,5-a][1,4]diazepine-2,5(6H)-dicarboxylate: To a mixture of 5-tert-butyl 2-methyl 3-iodo-7,8-dihydro-4H-pyrazolo[1,5-a][1,4]diazepine-2,5(6H)-dicarboxylate (2 g, 1 equiv), 4,4,5,5-tetramethyl-2-(prop-1-en-2-yl)-1,3,2-dioxaborolane (1.60 g, 2 equiv) and Cs2CO3 (4.64 g, 3 equiv) in dioxane (12 mL) and H₂O (3 mL) was degassed and purged with N2 3 times. Pd(dppf)C12 (347 mg, 0.1 equiv) was added, and the mixture was stirred at 90° C. for 12 hours under N2 atmosphere. The mixture was diluted with H₂O (10 mL) and extracted with ethyl acetate (2×10 mL). The combined organic layers were dried over anhydrous Na₂SO₄, concentrated, and purified with reversed phase flash chromatography [C18, 0.1% formic acid condition] to afford the tittle compound (1.35 g, 84% yield) as yellow oil; LCMS (ESI, M+1): m/z=336.1,

Step B. 5-tert-butyl 2-methyl 3-isopropyl-7,8-dihydro-4H-pyrazolo[1,5-a][1,4]diazepine-2,5(6H)-dicarboxylate: Pd/C (200 mg, 10% purity) was added into MeOH (10 mL) under N2 atmosphere. 5-tert-butyl 2-methyl 3-(prop-i-en-2-yl)-7,8-dihydro-4H-pyrazolo[1,5-a][1,4]diazepine-2,5(6H)-dicarboxylate (1.8 g, 1 equiv) was added and the mixture was degassed and purged with H2 3 times. The mixture was stirred at 25° C. for 12 hours under H2 (15 Psi) atmosphere. The mixture was filtered through a pad of Celite. The filter cake was washed with MeOH (50 mL). The filtrate was concentrated to afford the title compound (1.75 g, crude) as light-yellow oil; LCMS (ESI, M+1): m/z=338.2.

Step C. 5-(tert-butoxycarbonyl)-3-isopropyl-5,6,7,8-tetrahydro-4H-pyrazolo[1,5-a][1,4]diazepine-2-carboxylic acid: To a solution of 5-tert-butyl 2-methyl 3-isopropyl-7,8-dihydro-4H-pyrazolo[1,5-a][1,4]diazepine-2,5(6H)-dicarboxylate (1.65 g, 1 equiv) in MeOH (10 mL) was added NaOH (998 mg, 5 equiv). The mixture was stirred at 25° C. for 12 hours. The pH of the mixture was adjusted to 3 with IM HCl below 10° C. The mixture was extracted with ethyl acetate (3×10 mL). The combined organic layers were dried over anhydrous Na₂SO₄, concentrated, and purified with reversed phase flash chromatography [C18, 0.1% formic acid condition] to afford the tittle compound (1.5 g, 94% yield) as white solid; LCMS (ESI, M+1): m/z =324.1.

Step D. tert-butyl 2-(dimethylcarbamoyl)-3-isopropyl-7,8-dihydro-4H-pyrazolo[1,5-a][1,4]diazepine-5(6H)-carboxylate: To a solution of 5-(tert-butoxycarbonyl)-3-isopropyl-5,6,7,8-tetrahydro-4H-pyrazolo[1,5-a][1,4]diazepine-2-carboxylic acid (500 mg, 1 equiv) and Me2NH (2 M in THF, 2.32 mL, 3 equiv) in DMF (4 mL) were added HATU (1.18 g, 2 equiv) and N,N-diethylpropan-2-amine (400 mg, 2 equiv). The mixture was stirred at 25° C. for 2 hours. The mixture was diluted with H₂O (10 mL) and extracted with ethyl acetate (2×10 mL). The combined organic layers were dried over anhydrous Na₂SO₄, concentrated, and purified with reversed phase flash chromatography [C18, 0.1% formic acid condition] to afford the tittle compound (450 mg, 82% yield) as red oil; LCMS (ESI, M+1): m/z=351.2.

Step E. 3-isopropyl-N,N-dimethyl-5,6,7,8-tetrahydro-4H-pyrazolo[1,5-a][1,4]diazepine-2-carboxamide: To a solution of tert-butyl 2-(dimethylcarbamoyl)-3-isopropyl-7,8-dihydro-4H-pyrazolo[1,5-a][1,4]diazepine-5(6H)-carboxylate (250 mg, 1 equiv) in MeOH (2 mL) was added HCl•MeOH (4 M, 2 mL). The mixture was stirred at 25° C. for 2 hours. The reaction was concentrated. The residue was dissolved in MeOH (3 mL). Na2HCO3 (1 g) was added, and the mixture was stirred for 0.5 hours. The mixture was filtered. The filter cake was washed with MeOH (10 mL). The filtrate was concentrated to afford the title compound (150 mg, crude) as red oil; LCMS (ESI, M+1): m/z=251.1.

The last two steps were performed according to Example 248. The title compound was obtained as white solid. 1H NMR (400 MHz, METHANOL-d4) δ=7.50 (dd, J=5.6, 9.2 Hz, 1H), 7.14 (t, J=9.4 Hz, 1H), 6.99-6.94 (m, 2H), 5.39-5.33 (m, 1H), 5.33-5.17 (m, 1H), 4.58 (br dd, J=4.4, 16.2 Hz, 1H), 4.52-4.39 (m, 2H), 4.16-4.01 (m, 4H), 3.76 (ddd, J=3.2, 10.0, 14.0 Hz, 1H), 3.69-3.61 (m, 1H), 3.48-3.35 (m, 3H), 3.26-3.16 (m, 3H), 3.16-3.11 (m, 2H), 3.08 (s, 3H), 3.04-2.97 (m, 2H), 2.95 (s, 3H), 2.63 (br d, J=12.8 Hz, 1H), 2.32-1.78 (m, 9H), 1.21 (d, J=7.2 Hz, 3H), 1.17 (d, J=7.2 Hz, 3H), 1.11 (t, J=7.2 Hz, 3H); LCMS (ESI, M+1). m/z=729.3.

### Example 296

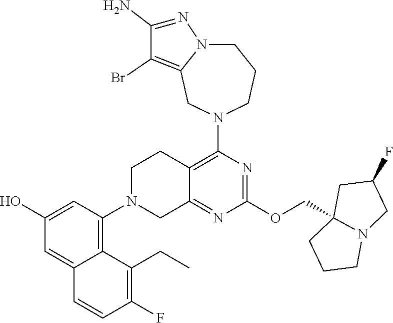 [SMILES: CCc1c(F)ccc2cc(O)cc(N3CCc4c(nc(OC[C@@]56CCCN5C[C@H](F)C6)nc4N4CCCn5nc(N)c(Br)c5C4)C3)c12]

### 4-(4-(2-amino-3-bromo-7,8-dihydro-4H-pyrazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

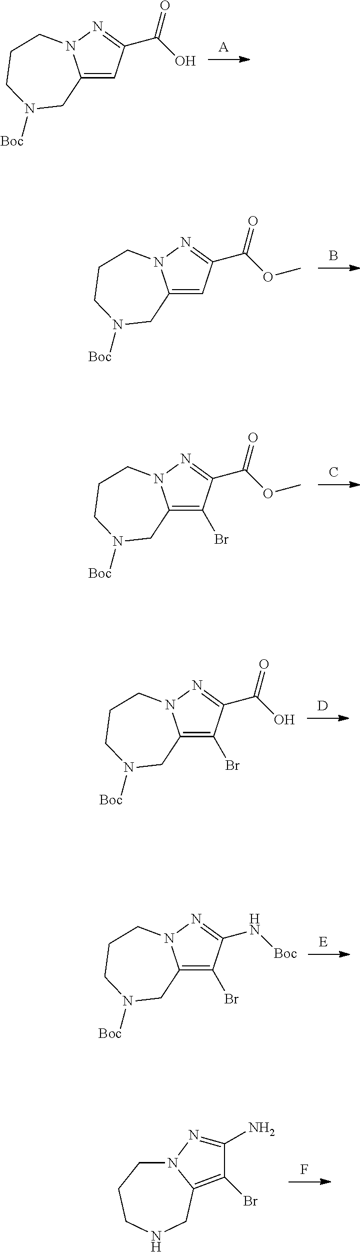

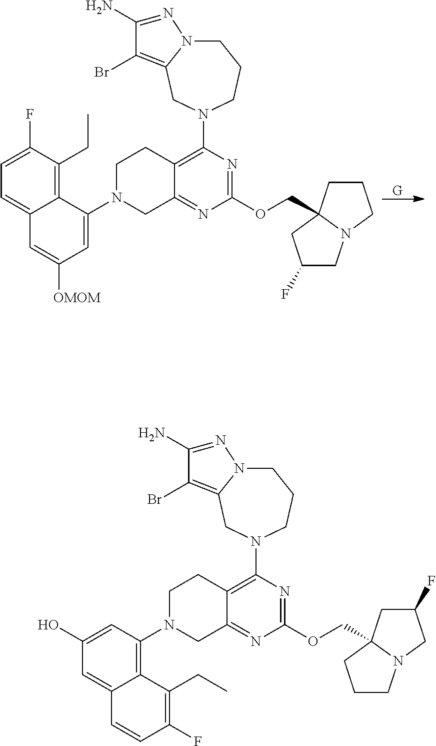

Step A. 5-tert-butyl 2-methyl 7,8-dihydro-4H-pyrazolo[1,5-a][1,4]diazepine-2,5(6H)-dicarboxylate: To a solution of 5-tert-butoxycarbonyl-4,6,7,8-tetrahydropyrazolo[1,5-a][1,4]diazepine-2-carboxylic acid (2.00 g, 1.0 equiv) in MeOH (10 mL) and DCM (20 mL) was added diazomethyl(trimethyl) silane (2 M, 2.0 equiv). The mixture was washed with saturated NaHCO₃ solution (50 mL) and extracted with EtOAc (30 mL×2). The combined organic layers were washed with brine (30 mL), dried over anhydrous sodium sulfate, and concentrated to afford the title compound (1.92 g, 90% yield) as white solid. LCMS (ESI, M+1): m/z=296.1

Step B. 5-tert-butyl 2-methyl 3-bromo-7,8-dihydro-4H-pyrazolo[1,5-a][1,4]diazepine-2,5(6H)-dicarboxylate: To a solution of 05-tert-butyl 02-methyl 4,6,7,8-tetrahydropyrazolo[1,5-a][1,4]diazepine-2,5-dicarboxylate (1.00. g, 1.0 equiv) in AcOH (10 mL) and ACN (5 mL) was added NBS (1.21 g, 2.0 equiv). The mixture was stirred at 40° C. for 5 hours. NaHCO₃ solution was added dropwise to the mixture (200 mL) slowly and then was extracted with EtOAc (2×100 mL). The combined organic layers were washed with brine (100 mL), dried over anhydrous sodium sulfate, concentrated, and purified by column chromatography (SiO2, Petroleum ether/Ethyl acetate=I/O to Petroleum ether/Ethyl acetate=0/1) to afford the title compound (1.20 mg, 92% yield) as yellow oil; LCMS (ESI, M+1): m/z=374.0

Step C. 3-bromo-5-(tert-butoxycarbonyl)-5,6,7,8-tetrahydro-4H-pyrazolo[1,5-a][1,4]diazepine-2-carboxylic acid: To a solution of 05-tert-butyl 02-methyl 3-bromo-4,6,7,8-tetrahydropyrazolo[1,5-a][1,4]diazepine-2,5-dicarboxylate (1.20 g, 1.0 equiv) in MeOH (12 mL) and H₂O (4 mL) was added LiOH (384 mg, 5.0 equiv). The mixture was stirred at 20° C. for 16 hours. The reaction was washed with EtOAc (20 mL×2). The aqueous phase was acidified by 2M HCl solution (50 mL), and then extracted with EtOAc (20 mL×3). The combined organic layers were washed with brine (30 mL), dried over anhydrous sodium sulfatesulfate, and concentrated to afford the title compound (803 mg, 69% yield) as white solid. LCMS (ESI, M+1): m/z=360.0

Step D. tert-butyl 3-bromo-2-((tert-butoxycarbonyl)amino)-7,8-dihydro-4H-pyrazolo[1,5-a][1,4]diazepine-5(6H)-carboxylate: To a mixture of 3-bromo-5-(tert-butoxycarbonyl)-5,6,7,8-tetrahydro-4H-pyrazolo[1,5-a][1,4]diazepine-2-carboxylic acid (703 mg, 1.0 equiv) in toluene (6 mL) were added 4 A molecular sieve (200 mg, 1.0 equiv), TEA (815 L, 3.0 equiv), DPPA (1.5 equiv) and 2-methylpropan-2-ol (4.34 g, 30 equiv) and was stirred at 110° C. for 16 hours under nitrogen. The mixture was filtered and the filtrate was diluted with H₂O (30 mL) and extracted with EtOAc (2×20 mL). The combined organic layers were washed with brine (20 mL), dried over anhydrous sodium sulfate, concentrated, and purified by column chromatography [column SiO2: Welch Ultimate XB-SiOH 250×50×10 m; A: Hexane, B: EtOH (0.1% FA), B %: 1%-30%, over 15 min] to afford the title compound (366 mg, 43% yield) as yellow oil.

Step E. 3-bromo-5,6,7,8-tetrahydro-4H-pyrazolo[1,5-a][1,4]diazepin-2-amine: To a solution of tert-butyl 3-bromo-2-(tert-butoxycarbonylamino)-4,6,7,8-tetrahydropyrazolo[1,5-a][1,4]diazepine-5-carboxylate (200 mg, 1.0 equiv) in MeOH (1 mL) was added HCl-MeOH (4 M, 5 mL). The mixture was stirred at 25° C. for 1 hour. The reaction was concentrated to afford the title compound (803 mg, 69% yield) as white solid; 1H NMR (400 MHz, METHANOL-d4) 6 =7.46-7.32 (m, 1H), 7.29-7.14 (m, 1H), 4.61-4.47 (m, 4H), 3.66-3.57 (m, 2H), 2.20 (br s, 2H).

The last two steps were performed according to Example 248. The title compound was obtained as yellow solid (FA salt). 1H NMR (400 MHz, METHANOL-d4) δ=7.50 (dd, J=5.6, 9.2 Hz, 1H), 7.14 (t, J=9.2 Hz, 1H), 7.00-6.93 (m, 2H), 5.45-5.26 (m, 1H), 4.78-4.63 (m, 2H), 4.07 (s, 5H), 4.01-3.84 (m, 2H), 3.73-3.60 (m, 1H), 3.52-3.44 (m, 2H), 3.43-3.35 (m, 4H), 3.20-3.06 (m, 2H), 2.74-2.63 (m, 1H), 2.47-1.85 (m, 9H), 1.15-1.07 (m, 3H); LCMS (ESI, M+1): m/z=711.3;

### Example 297

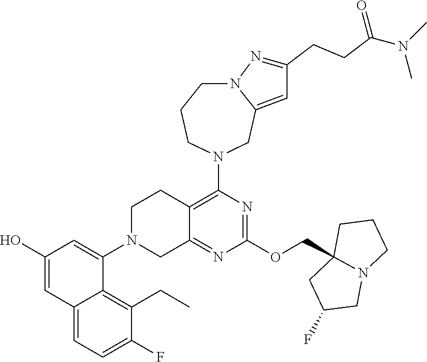 [SMILES: CCc1c(F)ccc2cc(O)cc(N3CCc4c(nc(OC[C@@]56CCCN5C[C@H](F)C6)nc4N4CCCn5nc(CCC(=O)N(C)C)cc5C4)C3)c12]

### 3-(5-(7-(8-ethyl-7-fluoro-3-hydroxynaphthalen-1-yl)-2-(((2R,7aS)-2-fluorohexahydro-1H-pyrrolizin-7a-yl)methoxy)-5, 6,7, 8-tetrahydropyrido[3,4-d]pyrimidin-4-yl)-5, 6,7, 8-tetrahydro-4H-pyrazolo[1,5-a][1,4]diazepin-2-yl)-N,N-dimethylpropanamide

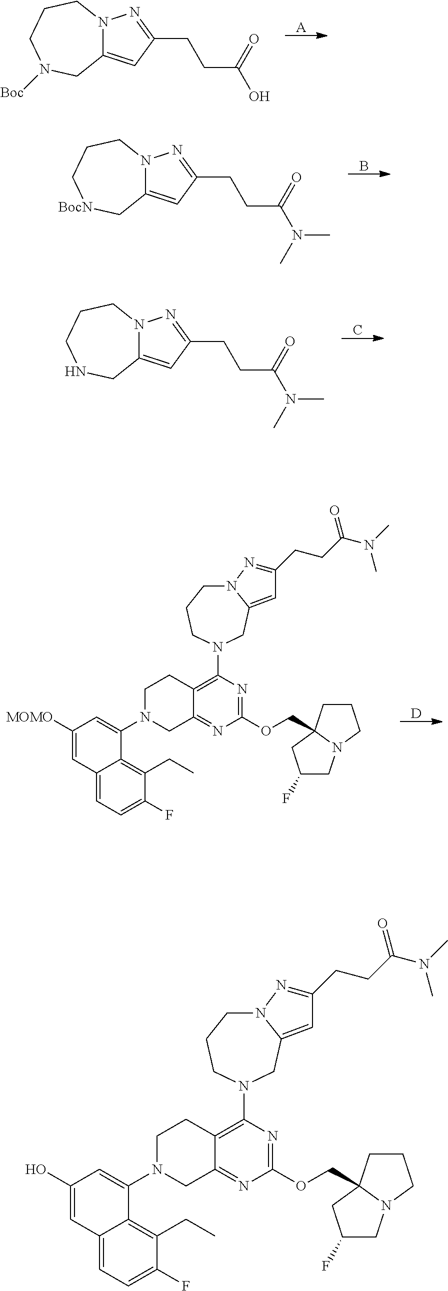

Step A. tert-butyl 2-(3-(dimethylamino)-3-oxopropyl)-7,8-dihydro-4H-pyrazolo[1,5-a][1,4]diazepine-5(6H)-carboxylate: To a solution of 3-(5-(tert-butoxycarbonyl)-5,6,7,8-tetrahydro-4H-pyrazolo[1,5-a][1,4]diazepin-2-yl)propanoic acid (300 mg, 1.0 equiv) in THF (6.0 mL) were added HATU (553 mg, 1.5 equiv), N,N-diethylpropan-2-amine (376 mg, 3.0 equiv) and (CH₃)₂NH (727 μL, 2.0 M, 1.5 equiv). The mixture was stirred at 20° C. for 2 hours. The reaction was diluted with water (20 mL) and extracted with DCM (3×20 mL). The combined organic layers were washed with brine (20 mL), dried over Na₂SO₄, concentrated, and purified with prep-HPLC [column: Phenomenex luna C18 150×40 mm×15 μm; mobile phase: [water (FA)-ACN]; B %: 23%-53%, 10 minutes] to afford the title compound (100 mg, 31% yield) as white solid. LCMS (ESI, M+1): m/z=337.1.

Step B. N,N-dimethyl-3-(5,6,7,8-tetrahydro-4H-pyrazolo[1,5-a][1,4]diazepin-2-yl)propanamide: To a solution of tert-butyl 2-(3-(dimethylamino)-3-oxopropyl)-7,8-dihydro-4H-pyrazolo[1,5-a][1,4]diazepine-5(6H)-carboxylate (100 mg, 1.0 equiv) was added HCl/MeOH (4.0 mL, 4.0 M). The mixture was stirred at 20° C. for 1 hour. The reaction was concentrated under reduced pressure to give a residue. The residue was purified with prep-HPLC [column: Waters Xbridge 150×25 mm×5 μm; mobile phase: [water (NH4HCO3)-ACN]; B %: 2%-32%, 8 minutes] to afford the title compound (50.0 mg, 71% yield) as white solid. LCMS (ESI, M+1): m/z=237.1.

The last two steps were performed according to Example 248. The title compound was obtained as white solid. 1H NMR (400 MHz, METHANOL-d4) δ=7.51 (dd, J=6.0, 9.2 Hz, 1H), 7.14 (t, J=9.2 Hz, 1H), 7.00-6.87 (m, 2H), 6.05 (d, J=1.6 Hz, 1H), 5.35-5.17 (m, 1H), 4.95 (br d, J=16.0 Hz, 1H), 4.78-4.69 (m, 1H), 4.47-4.34 (m, 2H), 4.26-3.82 (m, 5H), 3.70 (br d, J=18.8 Hz, 1H), 3.56-3.34 (m, 3H), 3.24-3.09 (m, 5H), 3.03-2.95 (m, 4H), 2.90 (s, 3H), 2.82 (br d, J=8.0 Hz, 2H), 2.74-2.63 (m, 3H), 2.32-2.12 (m, 3H), 2.09-1.82 (m, 5H), 1.18-1.05 (m, 3H); LCMS (ESI, M+1): m/z=715.4.

### Example 298

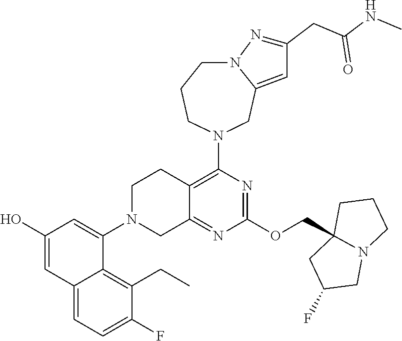 [SMILES: CCc1c(F)ccc2cc(O)cc(N3CCc4c(nc(OC[C@@]56CCCN5C[C@H](F)C6)nc4N4CCCn5nc(CC(=O)NC)cc5C4)C3)c12]

### 2-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-methylacetamide

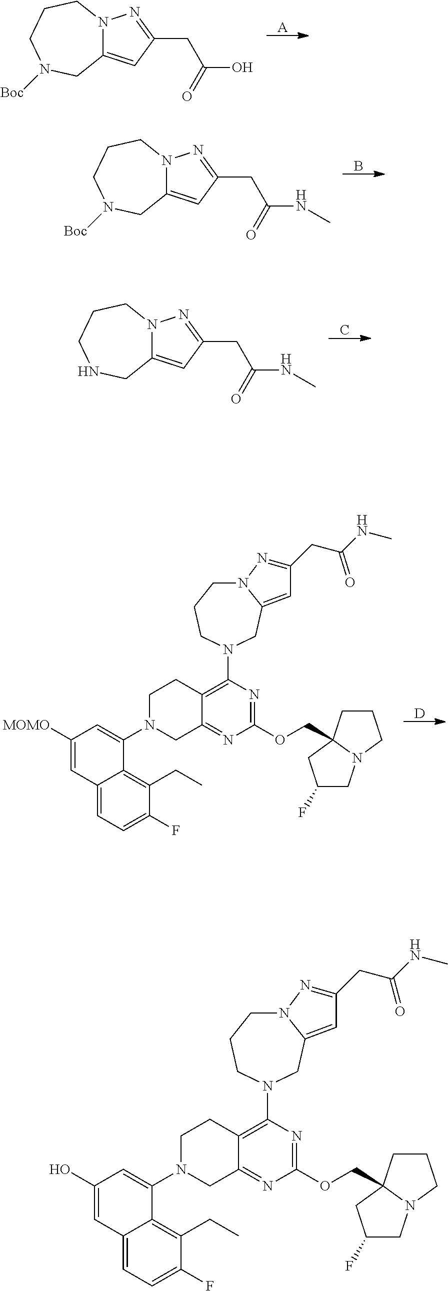

Step A. tert-butyl 2-(2-(methylamino)-2-oxoethyl)-7,8-dihydro-4H-pyrazolo[1,5-a][1,4]diazepine-5(6H)-carboxylate: To a solution of 2-(5-(tert-butoxycarbonyl)-5,6,7,8-tetrahydro-4H-pyrazolo[1,5-a][1,4]diazepin-2-yl)acetic acid (200 mg, 1.0 equiv) in THE (6.0 mL) were added HATU (386 mg, 1.5 equiv), N,N-diethylpropan-2-amine (262 mg, 3.0 equiv) and MeNH2 (8.37 mL, 2.0 M, 25 equiv). The mixture was stirred at 20° C. for 2 hours. The reaction was quenched by NH₄Cl solution (20 mL) at 20° C., and then extracted with DCM (3×20 mL). The combined organic layers were washed with brine (20 mL), dried over Na₂SO₄, and concentrated to afford the title compound (200 mg, crude) as yellow oil. LCMS (ESI, M+1): m/z =309.1.

Step B. N-methyl-2-(5,6,7,8-tetrahydro-4H-pyrazolo[1,5-a][1,4]diazepin-2-yl)acetamide: To a solution of tert-butyl 2-(2-(methylamino)-2-oxoethyl)-7,8-dihydro-4H-pyrazolo[1,5-a][1,4]diazepine-5(6H)-carboxylate (200 mg, 1.0 equiv) in MeOH (2.0 mL) was added HCl/MeOH (1.33 mL, 4.0 M). The mixture was stirred at 20° C. for 1 hour. The reaction was concentrated to afford the title compound (200 mg, crude) as yellow oil. LCMS (ESI, M+1): m/z=209.1.

The last two steps were performed according to Example 248. The title compound was obtained as white solid. 1H NMR (400 MHz, METHANOL-d4) δ=7.51 (dd, J=6.0, 8.8 Hz, 1H), 7.15 (t, J=9.2 Hz, 1H), 6.96 (br d, J=4.4 Hz, 2H), 6.15 (s, 1H), 5.33-5.19 (m, 1H), 5.00-4.93 (m, 2H), 4.80-4.72 (m, 2H), 4.64-4.55 (m, 2H), 4.50-4.34 (m, 2H), 4.27-3.86 (m, 5H), 3.76-3.62 (m, 1H), 3.56-3.36 (m, 5H), 3.14 (br s, 1H), 3.25-3.13 (m, 1H), 3.03-2.94 (m, 1H), 2.73 (s, 3H), 2.28 (br s, 5H), 2.00-1.84 (m, 3H), 1.11 (br t, J=6.8 Hz, 3H); LCMS (ESI, M+1): m/z=687.5 EXAMPLE 299

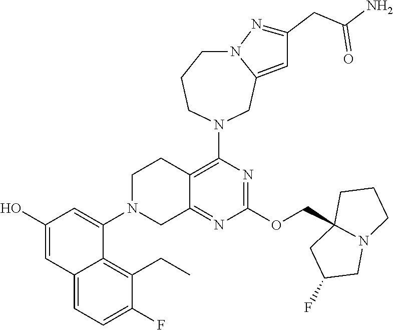 [SMILES: CCc1c(F)ccc2cc(O)cc(N3CCc4c(nc(OC[C@@]56CCCN5C[C@H](F)C6)nc4N4CCCn5nc(CC(N)=O)cc5C4)C3)c12]

### 2-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)acetamide

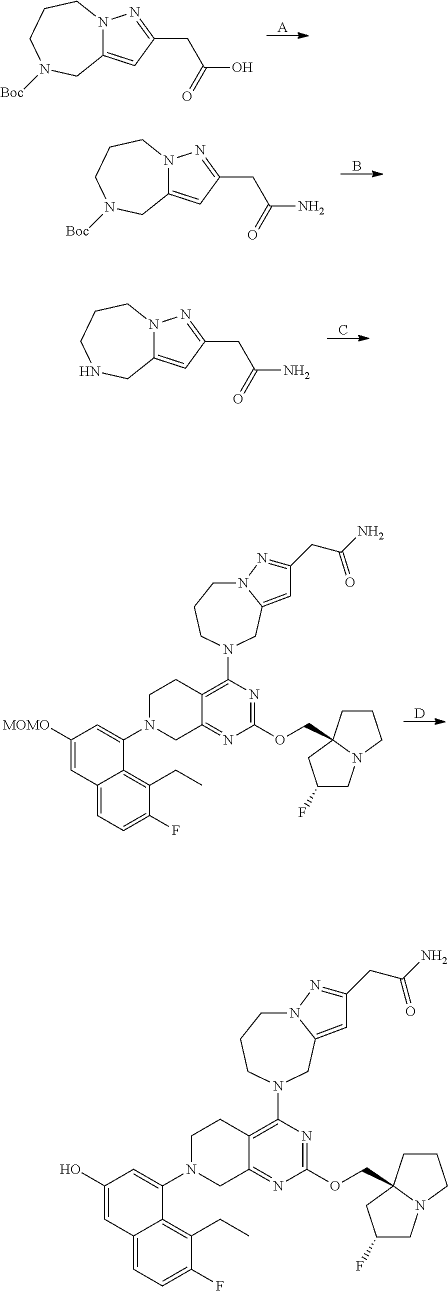

Synthesized according to Example 298 except that NH₄C1 instead of MeNH2 was used in the first step. The title compound was obtained as yellow solid. 1H NMR (400 MHz, METHANOL-d4) δ=7.51 (dd, J=6.0, 8.8 Hz, 1H), 7.14 (t, J=9.2 Hz, 1H), 6.96 (br d, J=5.2 Hz, 2H), 6.19 (s, 1H), 5.40-5.23 (m, 1H), 4.84-4.73 (m, 2H), 4.44 (br s, 2H), 4.24-3.99 (m, 5H), 3.69 (br d, J=18.4 Hz, 1H), 3.54-3.34 (m, 6H), 3.27 (br s, 1H), 3.18 (br d, J=8.8 Hz, 2H), 3.07 (br d, J=5.6 Hz, 1H), 2.72 (br d, J=10.0 Hz, 1H), 2.44-1.81 (m, 9H), 1.11 (br t, J=7.2 Hz, 3H); LCMS (ESI, M+1). m/z=673.4.

### Example 300

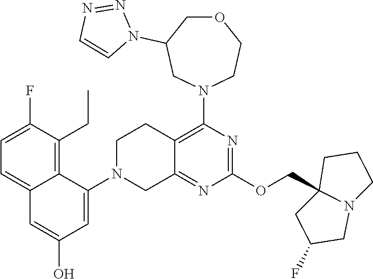 [SMILES: CCc1c(F)ccc2cc(O)cc(N3CCc4c(nc(OC[C@@]56CCCN5C[C@H](F)C6)nc4N4CCOCC(n5ccnn5)C4)C3)c12]

### 4-(4-(6-(1H-1,2,3-triazol-1-yl)-1,4-oxazepan-4-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

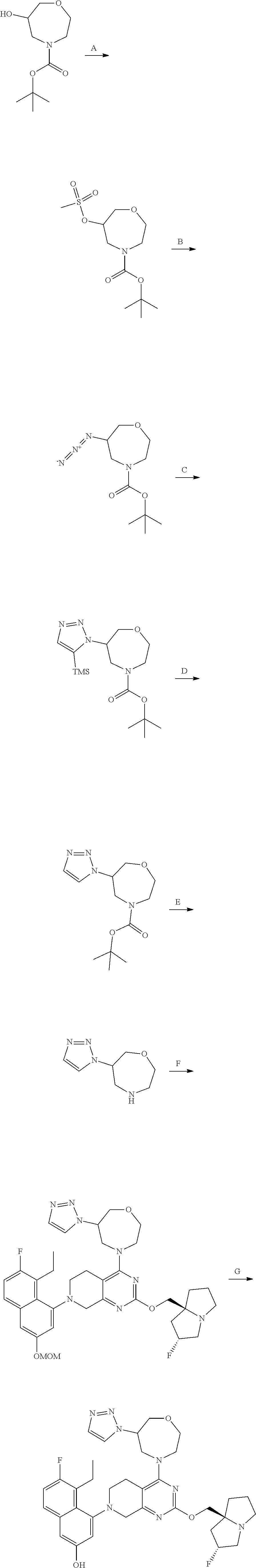

Step A. tert-butyl 6-((methylsulfonyl)oxy)-1,4-oxazepane-4-carboxylate: To a solution of tert-butyl 6-hydroxy-1,4-oxazepane-4-carboxylate (2 g, 1.0 equiv) and TEA (2.33 g, 2.5 equiv) in THE (20 mL) was added MsC1 (2.9 g, 2.8 equiv) dropwise at 0° C. The mixture was stirred at 0° C. for 1 hour. The resulting mixture was quenched with saturated NaHCO₃ aqueous (5 mL) at 0° C., diluted with H₂O (40 mL), and extracted with ethyl acetate (3×30 mL). The combined organic layers were dried over Na₂SO₄, filtered, concentrated, and purified by column chromatography [SiO2, petroleum ether/ethyl acetate=10/1 to 1/2] to afford the title compound (2.8 g, 98% yield) as off-white solid; 1H NMR (400 MHz, DMSO-d6) δ=4.84 (br d, J=4.4 Hz, 1H), 3.99-3.34 (m, 8H), 3.20 (s, 3H), 1.42 (s, 9H)

Step B. tert-butyl 6-azido-1,4-oxazepane-4-carboxylate: To a solution of tert-butyl 6-methylsulfonyloxy-1,4-oxazepane-4-carboxylate (1.8 g, 1.0 equiv) in DMF (18 mL) was added NaN3 (2.46 g, 6.2 equiv) slowly. The mixture was stirred at 70° C. for 12 hours. To the reaction was added saturated Na₂CO₃ aqueous until about pH 9 at 0° C. The reaction was quenched by addition of H₂O (40 mL) and extracted with ethyl acetate (3×40 mL). The combined organic layers were washed with H₂O (40 mL), dried over Na₂SO₄, filtered, concentrated, and purified by column chromatography [SiO2, Petroleum ether/Ethyl acetate=100/0 to 8/1] to afford the title compound (1.3 g, 84% yield) as a brown oil; 1H NMR (400 MHz, DMSO-d6) δ=3.85-3.35 (m, 9H), 1.41 (d, J=6.4 Hz, 9H)

Step C. tert-butyl 6-(5-(trimethylsilyl)-1H-1,2,3-triazol-1-yl)-1,4-oxazepane-4-carboxylate: To a solution of tert-butyl 6-azido-1,4-oxazepane-4-carboxylate (1.2 g, 1.0 equiv) in THF (12 mL) were added CuI (94 mg, 0.1 equiv), TEA (200 mg, 0.4 equiv) and ethynyl(trimethyl)silane (1.95 g, 4.0 equiv) under N2 atmosphere. The reaction was stirred at 50° C. for 12 hours under N2 atmosphere. The reaction was filtered by Celite. The resulting mixture was diluted with water (40 mL) and extracted with ethyl acetate (3×20 mL). The organic phase was dried over Na₂SO₄, filtered, concentrated, and purified by reversed-phase [0.1% FA condition] to afford the title compound (1.55 g, 80% yield) as a brown oil; 1H NMR (400 MHz, METHANOL-d4) δ=8.08 (s, 1H), 3.92-3.41 (m, 9H), 1.53-1.43 (m, 9H), 0.31 (s, 9H)

Step D. tert-butyl 6-(1H-1,2,3-triazol-1-yl)-1,4-oxazepane-4-carboxylate: To a solution of tert-butyl 6-(5-(trimethylsilyl)-1H-1,2,3-triazol-1-yl)-1,4-oxazepane-4-carboxylate (1.3 g, 1.0 equiv) in THF (5 mL) was added TBAF (1 M, 15.27 mL, 4.0 equiv). The reaction was stirred at 45° C. for 2 hours. The reaction was diluted with water (40 mL) and extracted with ethyl acetate (3×20 mL). The organic phase was dried over Na₂SO₄, concentrated, and purified by reversed phase flash chromatography {₀0.1% FA condition] to afford the title compound (0.68 g, 66% yield) as brown solid; LCMS (ESI, M+1): m/z=269.1.

Step E. 6-(1H-1,2,3-triazol-1-yl)-1,4-oxazepane: To a solution of tert-butyl 6-(triazol-1-yl)-1,4-oxazepane-4-carboxylate (0.68 g, 1.0 equiv) in ACN (3 mL) was added HCl•dioxane (4 M, 7 mL, 11 equiv) slowly at 0° C. The reaction was stirred at 0° C. for 1 hour. The reaction was concentrated. The crude product was dissolved in MeOH (4 mL). NaHCO₃ (0.5 g) was added, and the mixture was stirred for 0.2 hours. The mixture was filtered, concentrated, and dissolved in [DCM:MeOH=10:1 (3×5 mL)]. The mixture was filtered and concentrated to afford the title compound (420 mg, 98.5% yield) as brown oil.

The last two steps were performed according to Example 248. The title compound was obtained as pink solid (FA salt). 1H NMR (400 MHz, METHANOL-d4) δ=8.52 (s, 1H), 8.21-8.14 (m, 1H), 7.81-7.75 (m, 11H), 7.56-7.46 (m, 11H), 7.15 (t, J=9.2 Hz, 1H), 7.03-6.93 (m, 2H), 5.70-5.56 (m, 1H), 5.51-5.32 (m, 1H), 4.81-4.66 (m, 1H), 4.60-4.42 (m, 1H), 4.40-4.14 (m, 5H), 4.09-3.95 (m, 3H), 3.93-3.74 (m, 2H), 3.68-3.58 (m, 1H), 3.56-3.46 (m, 3H), 3.38 (dt, J=2.4, 7.2 Hz, 2H), 3.26-3.05 (m, 3H), 2.81-2.68 (m, 1H), 2.53-2.30 (m, 2H), 2.29-2.19 (m, 1H), 2.17-2.07 (m, 2H), 2.06-1.94 (m, 1H), 1.11 (dt, J=2.8, 7.2 Hz, 3H); LCMS (ESI, M+1): m/z=647.5.

### Example 301

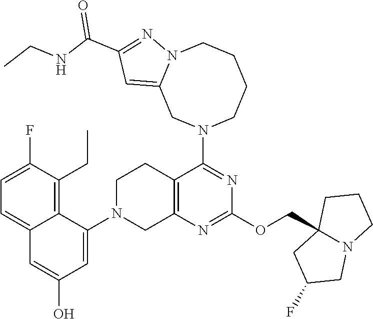 [SMILES: CCNC(=O)c1cc2n(n1)CCCCN(c1nc(OC[C@@]34CCCN3C[C@H](F)C4)nc3c1CCN(c1cc(O)cc4ccc(F)c(CC)c14)C3)C2]

### N-ethyl-5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-4,5,6,7,8,9-hexahydropyrazolo[1,5-a][1,4]diazocine-2-carboxamide

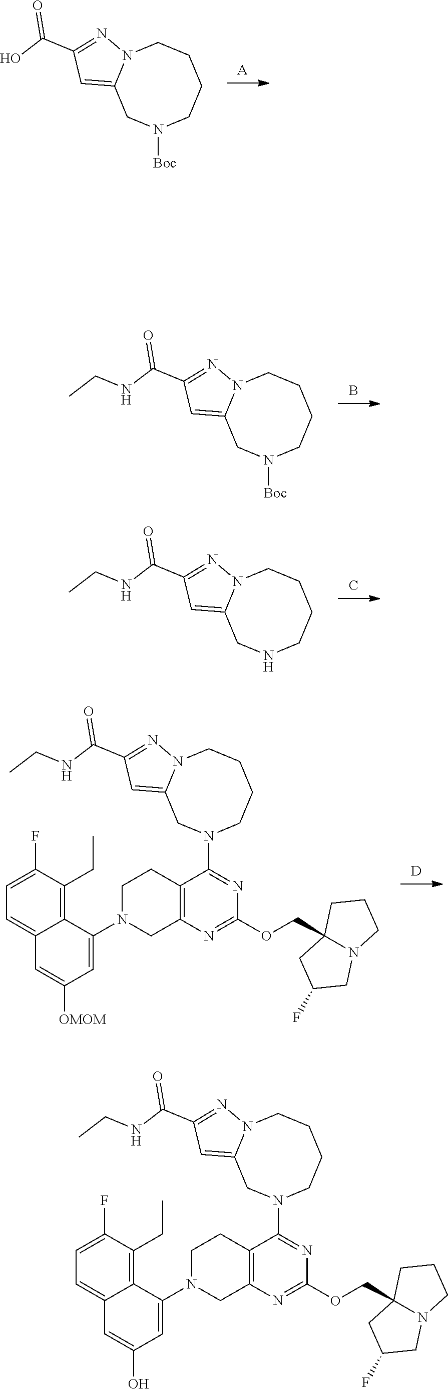

Step A. tert-butyl 2-(ethylcarbamoyl)-6,7,8,9-tetrahydropyrazolo[1,5-a][1,4]diazocine-5(4H)-carboxylate: To a solution of 5-tert-butoxycarbonyl-6,7,8,9-tetrahydro-4H-pyrazolo[1,5-a][1,4]diazocine-2-carboxylic acid (100 mg, 1.0 equiv) and ethanamine (15.3 mg, 1.0 equiv) in DMF (3.0 mL) were added HATU (386 mg, 3.0 equiv) and DIEA (438 mg, 10 equiv). The mixture was stirred at 40° C. for 12 hours. The reaction was diluted with water (30 mL) and extracted with ethyl acetate (30 mL). The combined organic layers were washed with brine (30 mL), dried over anhydrous sodium sulfate, filtered, and concentrated under reduced pressure to give a residue. The residue was purified by prep-TLC (SiO2, petroleum ether/ethyl acetate=0:1) to give the title compound (20 mg, 18% yield) as colorless oil; LCMS (ESI, M+1): m/z=323.2.

Step B. N-ethyl-4,5,6,7,8,9-hexahydropyrazolo[1,5-a][1,4]diazocine-2-carboxamide: A solution of tert-butyl 2-(ethylcarbamoyl)-6,7,8,9-tetrahydropyrazolo[1,5-a][1,4]diazocine-5(4H)-carboxylate (20 mg, 1.0 equiv) in MeCN (1.0 mL) and HCl/dioxane (4 M, 1.0 mL) was stirred at 25° C. for 1 hour. The reaction was concentrated to afford the title compound (50 mg, crude), LCMS (ESI, M+1): m/z=223.2.

The last two steps were performed according to Example 248. The title compound was obtained as yellow solid. 1H NMR (400 MHz, DMSO-d6) δ=9.71 (br s, 1H), 8.08 (t, J=6.0 Hz, 1H), 7.59 (dd, J=6.0, 8.8 Hz, 1H), 7.24 (t, J=9.2 Hz, 1H), 6.98 (s, 2H), 6.52 (s, 1H), 5.32-5.13 (m, 1H), 4.97-4.82 (m, 2H), 4.55-4.35 (m, 2H), 3.94-3.76 (m, 3H), 3.68-3.58 (m, 3H), 3.44-3.37 (m, 1H), 3.31-3.17 (m, 4H), 3.10-2.95 (m, 4H), 2.82-2.74 (m, 1H), 2.61-2.55 (m, 2H), 2.32 (s, 1H), 2.06-1.67 (m, 9H), 1.56-1.41 (m, 1H), 1.06 (q, J=6.8 Hz, 6H); LCMS (ESI, M+1): m/z=701.3.

### Example 302

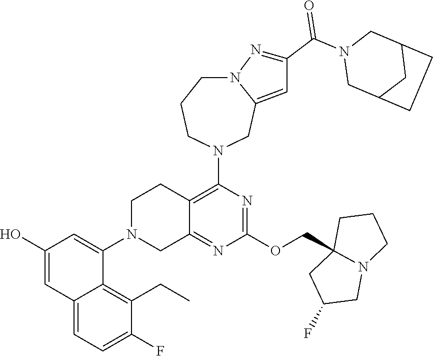 [SMILES: CCc1c(F)ccc2cc(O)cc(N3CCc4c(nc(OC[C@@]56CCCN5C[C@H](F)C6)nc4N4CCCn5nc(C(=O)N6CC7CCC(C7)C6)cc5C4)C3)c12]

### 3-azabicyclo[3.2.1]octan-3-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

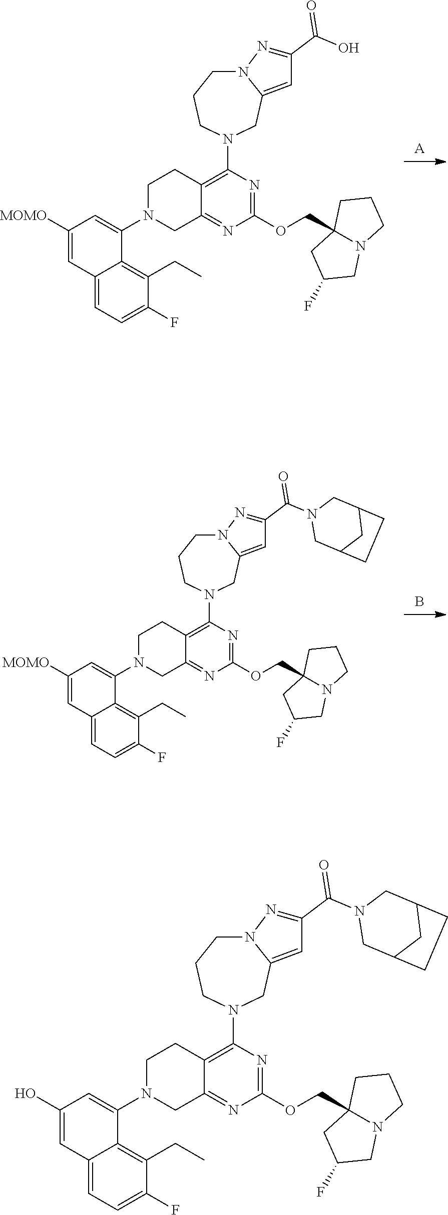

Synthesized according to Example 233 except that HCl instead of TFA was used in the last step. The title compound was obtained as off-white solid (FA salt). 1H NMR (400 MHz, METHANOL-d4) δ=7.53 (dd, J=5.6, 9.2 Hz, 1H), 7.16 (t, J=9.6 Hz, 1H), 6.99 (s, 2H), 6.66-6.51 (m, 1H), 5.49-5.25 (m, 1H), 5.11-4.95 (m, 1H), 4.90 (br s, 1H), 4.86-4.83 (m, 1H), 4.62-4.52 (m, 2H), 4.42 (br t, J=13.6 Hz, 1H), 4.36-4.13 (m, 4H), 4.07 (br dd, J=5.2, 17.2 Hz, 2H), 3.68 (br d, J=17.6 Hz, 1H), 3.61-3.53 (m, 1H), 3.41 (br d, J=4.0 Hz, 4H), 3.31-3.19 (m, 3H), 3.18-3.09 (m, 1H), 2.88 (br d, J=12.4 Hz, 1H), 2.82-2.71 (m, 1H), 2.44-2.15 (m, 6H), 2.13-1.87 (m, 4H), 1.79-1.50 (m, 6H), 1.13 (br dd, J=2.4, 4.4 Hz, 3H). LCMS (ESI, M+1). m/z=753.7.

### Example 303

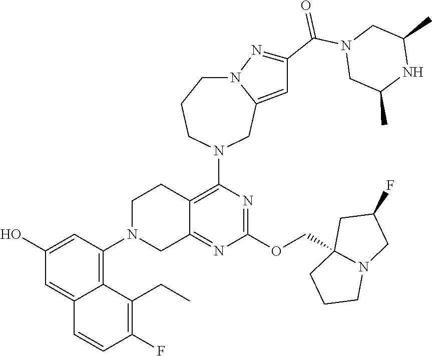 [SMILES: CCc1c(F)ccc2cc(O)cc(N3CCc4c(nc(OC[C@@]56CCCN5C[C@H](F)C6)nc4N4CCCn5nc(C(=O)N6C[C@@H](C)N[C@@H](C)C6)cc5C4)C3)c12]

### ((3 S,5R)-3,5-dimethylpiperazin-1-yl)(5-(7-(8-ethyl -7-fluoro-3-hydroxynaphthalen-1-yl)-2-(((2R,7aS)-2-fluorohexahydro-wH-pyrrolizin-7a-yl)methoxy)-5,6,7,8-tetrahydropyri do[3,4-d]pyrimidin-4-yl)-5,6,7, 8-tetrahydro-4H-pyrazolo[1, 5-a][1,4]diazepin-2-yl)methanone

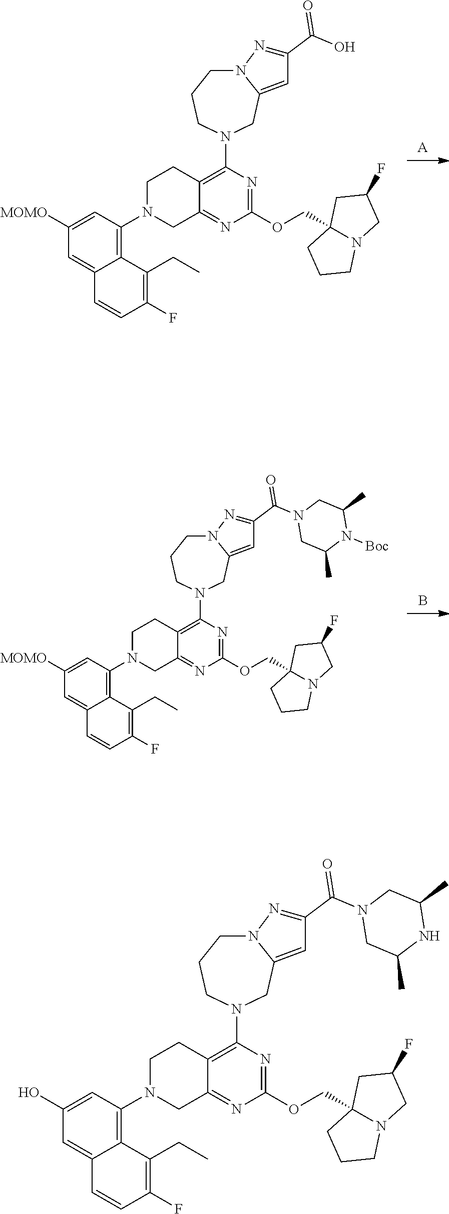

Synthesized according to Example 233 except that HCl instead of TFA was used in the last step. The title compound was obtained as white solid. 1H NMR (400 MHz, dimethylsulfoxide-d6) δ=9.68 (s, 1H), 7.66-7.52 (m, 1H), 7.24 (t, J=9.6 Hz, 1H), 6.99 (s, 2H), 6.48 (s, 1H), 5.34-5.12 (m, 1H), 5.00 (br d, J=15.2 Hz, 1H), 4.74 (br d, J=17.2 Hz, 1H), 4.62-4.43 (m, 3H), 4.41-4.29 (m, 1H), 4.24-4.06 (m, 1H), 3.95-3.76 (m, 4H), 3.60 (br d, J=16.8 Hz, 1H), 3.51-3.39 (m, 1H), 3.31-3.24 (m, 4H), 3.22-3.13 (m, 1H), 3.12-3.01 (m, 3H), 2.97 (br s, 1H), 2.85-2.74 (m, 1H), 2.64-2.59 (m, 2H), 2.27-2.10 (m, 3H), 2.08-2.03 (m, 1H), 2.00-1.90 (m, 3H), 1.83-1.68 (m, 3H), 1.06 (br t, J=6.8 Hz, 3H), 0.99 (br d, J=2.8 Hz, 3H), 0.92 (br s, 3H); 19F NMR (376 MHz, dimethylsulfoxide-d6) δ=−121.318, -171.999; LCMS (ESI, M+1). m/z=756.2.

### Example 304

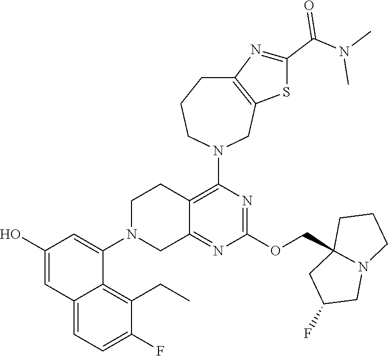 [SMILES: CCc1c(F)ccc2cc(O)cc(N3CCc4c(nc(OC[C@@]56CCCN5C[C@H](F)C6)nc4N4CCCc5nc(C(=O)N(C)C)sc5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-thiazolo[5,4-c]azepine-2-carboxamide

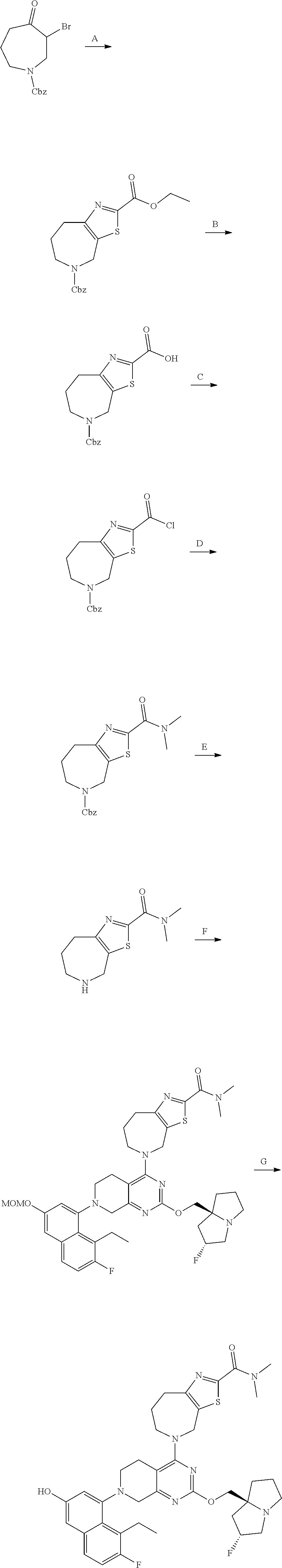

Step A. 5-benzyl 2-ethyl 7,8-dihydro-4H-thiazolo[5,4-c]azepine-2,5(6H)-dicarboxylate: To a solution of benzyl 3-bromo-4-oxoazepane-1-carboxylate (1.50 g, 1.0 equiv) in EtOH (15 mL) was added ethyl 2-amino-2-thioxoacetate (1.22 g, 2.0 equiv). The reaction was stirred at 80° C. for 24 hours. The mixture was filtered and purified by prep-HPLC (column: UniSil 10-120 C18 50×250 mm; mobile phase: [water (FA) -ACN]; B %: 35%-65%, 22 min) to afford the title compound (300 mg, 34% yield) as yellow oil; LCMS (ESI, M+1): m/z=361.2.

Step B. 5-((benzyloxy)carbonyl)-5,6,7,8-tetrahydro-4H-thiazolo[5,4-c]azepine-2-carboxylic acid: To a solution of 05-benzyl 02-ethyl 4,6,7,8-tetrahydrothiazolo[5,4-c]azepine-2,5-dicarboxylate (620 mg, 1.0 equiv) in THF (5 mL) and H₂O (1.5 mL) was added NaOH (688 mg, 10 equiv). The reaction was stirred at 20° C. for 0.5 hours. After completion, the pH of residue was adjusted to 4 with HCl (2 M). The mixture was extracted with ethyl acetate (3×10 mL). The combined organic layers were washed with saturated NaCl aqueous solution (2×15 mL), dried over Na₂SO₄, filtered, and concentrated under reduced pressure to afford the title compound (540 mg, 94% yield) as yellow oil.

Step C. benzyl 2-(chlorocarbonyl)-7,8-dihydro-4H-thiazolo[5,4-c]azepine-5(6H)-carboxylate: To a solution of 5-benzyloxycarbonyl-4,6,7,8-tetrahydrothiazolo[5,4-c]azepine-2-carboxylic acid (500 mg, 1.0 equiv) in DCM (1 mL) and DMF (0.1 mL) was added oxalyl dichloride (382 mg, 2.0 equiv) in DCM (1 mL). The reaction was stirred at 25° C. for 5 minutes. The reaction was concentrated to afford the title compound (520 mg, 98% yield) as yellow oil.

Step D. benzyl 2-(dimethylcarbamoyl)-7,8-dihydro-4H-thiazolo[5,4-c]azepine-5(6H)-carboxylate: To a solution of benzyl 2-(chlorocarbonyl)-7,8-dihydro-4H-thiazolo[5,4-c]azepine-5(6H)-carboxylate (560 mg, 1.0 equiv) in DCM (5 mL) was added dimethylamine (2 M, 1.60 mL, 2.0 equiv). The reaction was stirred at 20° C. for 10 minutes. The reaction was concentrated and purified by reversed-phase HPLC (0.1% FA condition) to afford the title compound (210 mg, 37% yield) as yellow oil; LCMS (ESI, M+1): m/z=360.6.

Step E. N,N-dimethyl-5,6,7,8-tetrahydro-4H-thiazolo[5,4-c]azepine-2-carboxamide: To a solution of benzyl 2-(dimethylcarbamoyl)-7,8-dihydro-4H-thiazolo[5,4-c]azepine-5(6H)-carboxylate (30.0 mg, 1.0 equiv) in DCM (0.5 mL) was added TMSI (735 mg, 44 equiv) at 0° C. The reaction was stirred at 20° C. for 1 hour. The mixture was diluted with water (5 mL) and washed with DCM (2×5 mL). The aqueous solution was lyophilized to afford the title compound (55.0 mg, crude) as yellow solid.

The last two steps were performed according to Example 248. The title compound was obtained as orange solid (FA salt). 1H NMR (400 MHz, METHANOL-d4) δ=7.51 (dd, J=5.6, 8.8 Hz, 1H), 7.14 (t, J=9.2 Hz, 1H), 6.97 (d, J=3.2 Hz, 2H), 5.45-5.21 (m, 1H), 5.10 (br dd, J=11.2, 16.0 Hz, 1H), 4.82-4.75 (m, 1H), 4.67-4.47 (m, 1H), 4.26-4.10 (m, 3H), 4.09-4.00 (m, 2H), 3.65 (br d, J=17.6 Hz, 1H), 3.59-3.47 (m, 4H), 3.38 (br d, J=5.8 Hz, 2H), 3.29-3.14 (m, 5H), 3.13-2.99 (m, 5H), 2.75 (br d, J=14.0 Hz, 1H), 2.32-2.09 (m, 4H), 2.07-1.85 (m, 4H), 1.11 (t, J=7.2 Hz, 3H); LCMS (ESI, M+1): m/z=704.3.

### Example 305

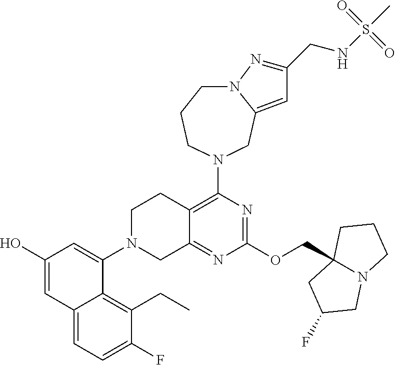 [SMILES: CCc1c(F)ccc2cc(O)cc(N3CCc4c(nc(OC[C@@]56CCCN5C[C@H](F)C6)nc4N4CCCn5nc(CNS(C)(=O)=O)cc5C4)C3)c12]

### N-((5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methyl)methanesulfonamide

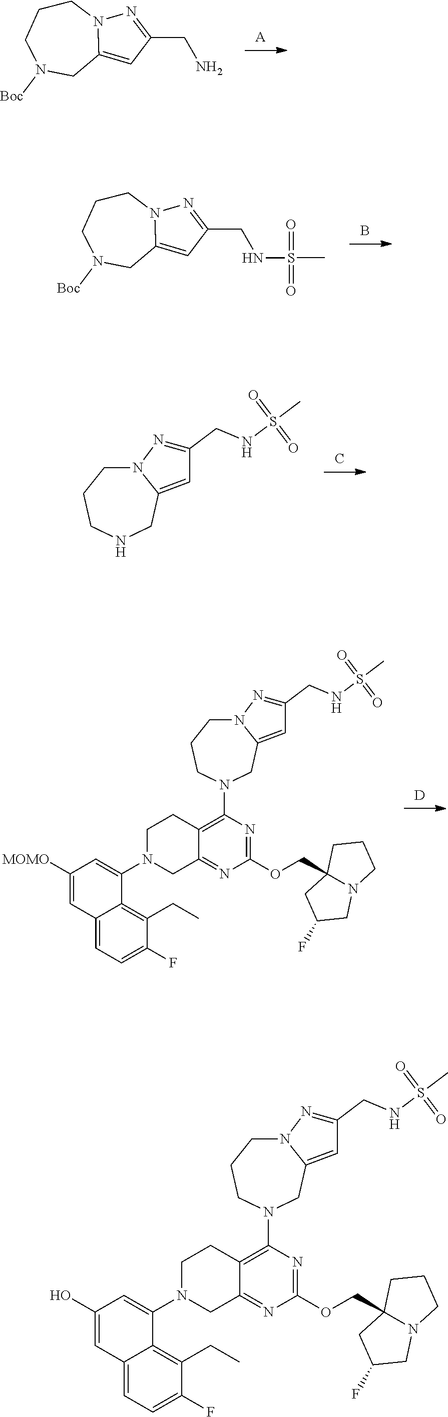

Step A. tert-butyl 2-(methylsulfonamidomethyl)-7,8-dihydro-4H-pyrazolo[1,5-a][1,4]diazepine-5(6H)-carboxylate: To a mixture of tert-butyl 2-(aminomethyl)-7,8-dihydro-4H-pyrazolo[1,5-a][1,4]diazepine-5(6H)-carboxylate (100 mg, 1.0 equiv) and TEA (114 mg, 3.0 equiv) in DCM (1 mL) was added methanesulfonyl chloride (0.370 g, 8.6 equiv) at 0° C. The reaction was stirred at 20° C. for 1 hour. The mixture was quenched with ice water (10 mL) and extracted with DCM (2×30 mL). The combined organic layers were washed with brine (10 mL), dried over anhydrous sodium sulfate, concentrated, and purified by reversed phase flash chromatography [water (FA 0.10%)/acetonitrile] to afford the title compound (80 mg, 62% yield) as yellow solid; LCMS (ESI, M+1): m/z=344.9.

Step B. N-((5,6,7,8-tetrahydro-4H-pyrazolo[1,5-a][1,4]diazepin-2-yl)methyl)methanesulfonamide: To a mixture of tert-butyl 2-(methylsulfonamidomethyl)-7,8-dihydro-4H-pyrazolo[1,5-a][1,4]diazepine-5(6H)-carboxylate (118 mg, 1.0 equiv) in MeCN (1 mL) was added HCl•dioxane (4 M, 5.8 mL, 67 equiv). The reaction was stirred at 25° C. for 1 hour. The mixture was concentrated to afford the title compound (80.6 mg, crude) as yellow solid and used into next step without further purification.

The last two steps were performed according to Example 248. The title compound was obtained as off-white solid. 1H NMR (400 MHz, METHANOL-d4) δ=7.49 (br d, J=1.2 Hz, 1H), 7.16-7.08 (m, 1H), 6.94 (s, 2H), 6.28 (d, J=2.8 Hz, 1H), 5.38-5.12 (m, 1H), 4.94-4.87 (m, 2H), 4.51-4.38 (m, 2H), 4.17 (s, 2H), 4.13-3.98 (m, 5H), 3.72 (br d, J=2.0 Hz, 1H), 3.54-3.45 (m, 1H), 3.44-3.34 (m, 2H), 3.25-3.09 (m, 5H), 2.98 (dt, J=5.6, 9.2 Hz, 1H), 2.83 (d, J=1.2 Hz, 3H), 2.75-2.65 (m, 1H), 2.30-2.11 (m, 3H), 2.11-1.91 (m, 4H), 1.89-1.78 (m, 1H), 1.12 (dt, J=2.0, 7.2 Hz, 3H), LCMS (ESI, M+1): m/z=723.3.

### Example 306

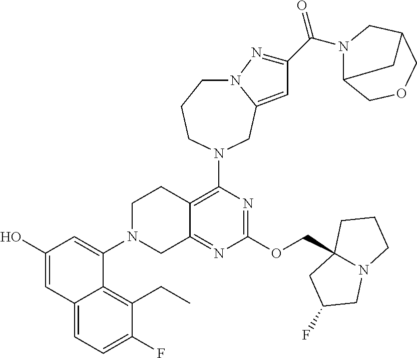 [SMILES: CCc1c(F)ccc2cc(O)cc(N3CCc4c(nc(OC[C@@]56CCCN5C[C@H](F)C6)nc4N4CCCn5nc(C(=O)N6CC7COCC6C7)cc5C4)C3)c12]

### 3-oxa-6-azabicyclo[3.2.1]octan-6-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

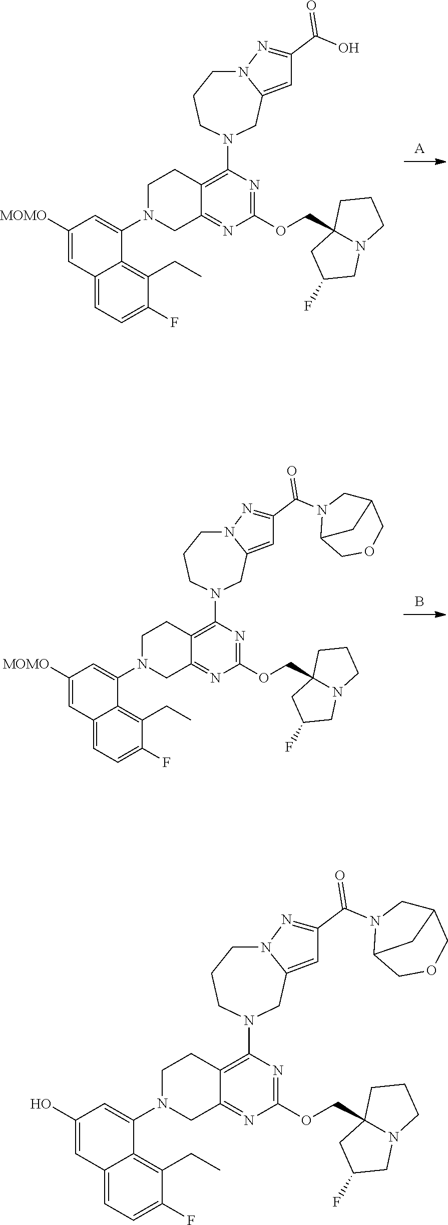

Synthesized according to Example 233 except that HCl instead of TFA was used in the last step. The title compound was obtained as yellow solid. 1HNMR (400 MHz, METHANOL-d4) δ=7.51 (dd, J=6.0, 8.8 Hz, 1H), 7.14 (t, J=9.2 Hz, 1H), 6.97 (s, 2H), 6.78-6.70 (m, 1H), 5.46-5.27 (m, 1H), 4.96 (br d, J=18.8 Hz, 2H), 4.62-4.39 (m, 3H), 4.32-3.89 (m, 7H), 3.81-3.62 (m, 4H), 3.55 (br t, J=11.6 Hz, 2H), 3.49-3.33 (m, 5H), 3.28-3.09 (m, 3H), 2.73 (br d, J=14.4 Hz, 1H), 2.46-2.17 (m, 5H), 2.14-1.88 (m, 6H), 1.11 (q, J=6.8 Hz, 3H); LCMS (ESI, M+1): m/z =755.9.

### Example 307

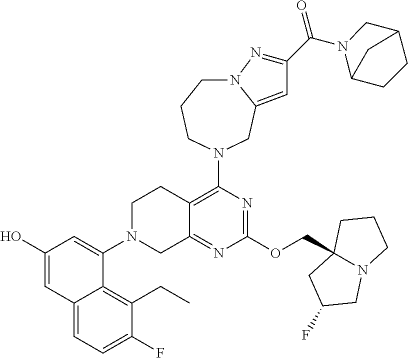 [SMILES: CCc1c(F)ccc2cc(O)cc(N3CCc4c(nc(OC[C@@]56CCCN5C[C@H](F)C6)nc4N4CCCn5nc(C(=O)N6CC7CCC6C7)cc5C4)C3)c12]

### 2-azabicyclo[2.2.1]heptan-2-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

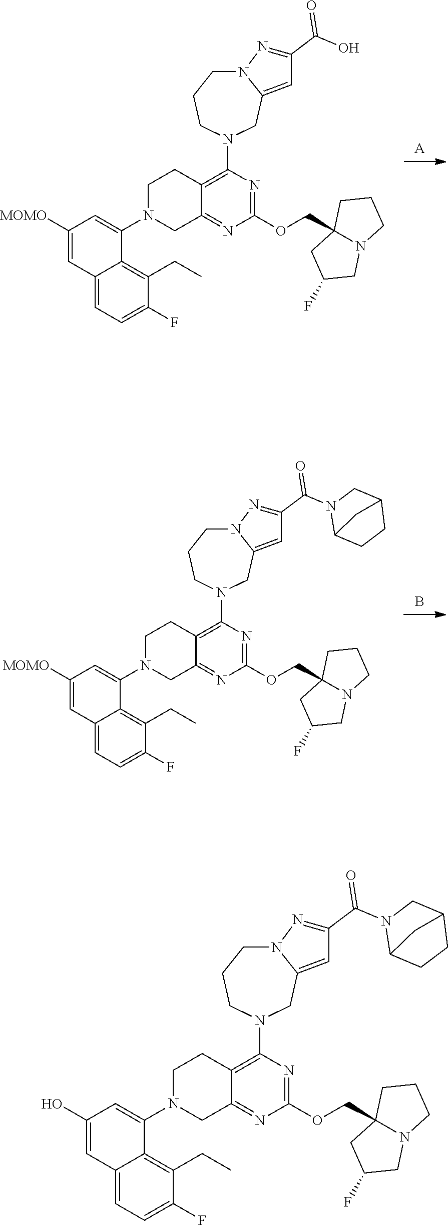

Synthesized according to Example 233 except that HCl instead of TFA was used in the last step. The title compound was obtained as orange solid (FA salt). 1H NMR (400 MHz, METHANOL-d4) δ=7.53 (dd, J=5.6, 8.8 Hz, 1H), 7.16 (t, J=9.2 Hz, 1H), 6.99 (s, 2H), 6.68 (br dd, J=3.2, 6.0 Hz, 1H), 5.48-5.24 (m, 1H), 5.21-4.86 (m, 2H), 4.74-4.67 (m, 1H), 4.60-4.49 (m, 2H), 4.29-3.97 (m, 5H), 3.87-3.61 (m, 2H), 3.59-3.46 (m, 2H), 3.45-3.36 (m, 3H), 3.25-2.95 (m, 5H), 2.83-2.70 (m, 1H), 2.66 (br s, 1H), 2.47-2.14 (m, 4H), 2.12-1.88 (m, 4H), 1.85-1.62 (m, 4H), 1.59-1.44 (m, 2H), 1.13 (br d, J=7.2 Hz, 3H). LCMS (ESI, M+1). m/z=739.5.

### Example 308

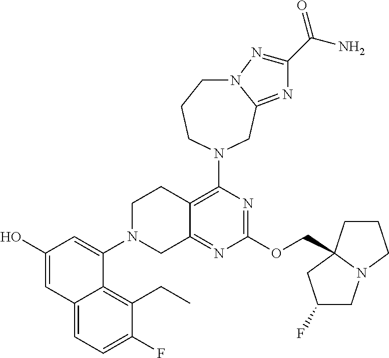 [SMILES: CCc1c(F)ccc2cc(O)cc(N3CCc4c(nc(OC[C@@]56CCCN5C[C@H](F)C6)nc4N4CCCn5nc(C(N)=O)nc5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepine-2-carboxamide

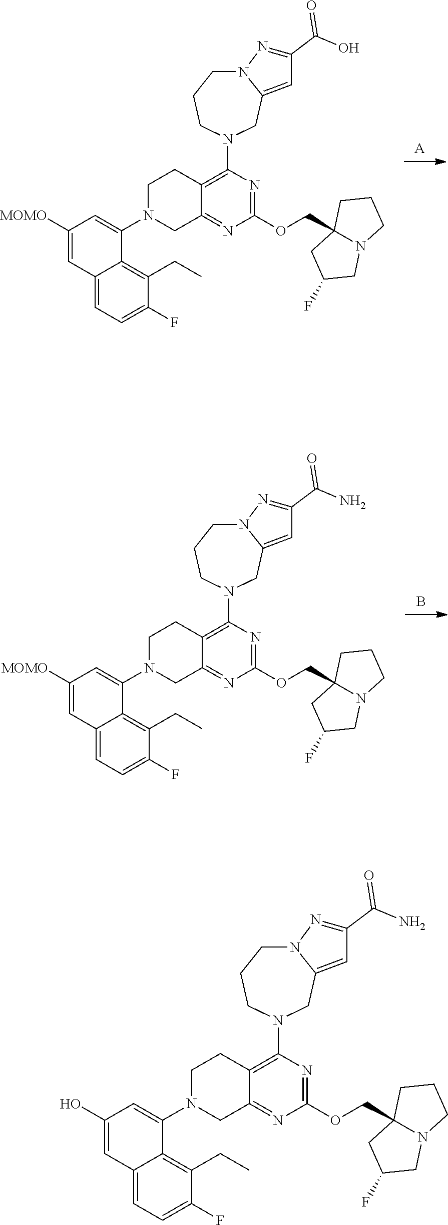

Synthesized according to Example 233 except that HCl instead of TFA was used in the last step. The title compound was obtained as white solid (FA salt). 1H NMR (400 MHz, METHANOL-d4) δ=7.53 (dd, J=5.6, 8.8 Hz, 1H), 7.16 (t, J=9.6 Hz, 1H), 6.93 (s, 2H), 6.74 (s, 1H), 5.49-5.27 (m, 1H), 5.04-4.94 (m, 1H), 4.84 (br s, 1H), 4.64-4.47 (m, 2H), 4.29-3.99 (m, 5H), 3.68 (br d, J=17.6 Hz, 1H), 3.56 (br d, J=9.2 Hz, 1H), 3.49 (br s, 5H), 3.29-3.09 (m, 3H), 2.76 (br d, J=14.0 Hz, 1H), 2.48-2.18 (m, 4H), 2.15-1.90 (m, 4H), 1.13 (t, J=7.2 Hz, 3H); LCMS (ESI, M+1). m/z=659.5.

### Example 309

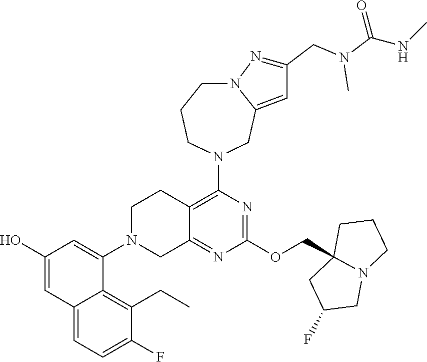 [SMILES: CCc1c(F)ccc2cc(O)cc(N3CCc4c(nc(OC[C@@]56CCCN5C[C@H](F)C6)nc4N4CCCn5nc(CN(C)C(=O)NC)cc5C4)C3)c12]

### 1-((5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methyl)-1,3-dimethylurea

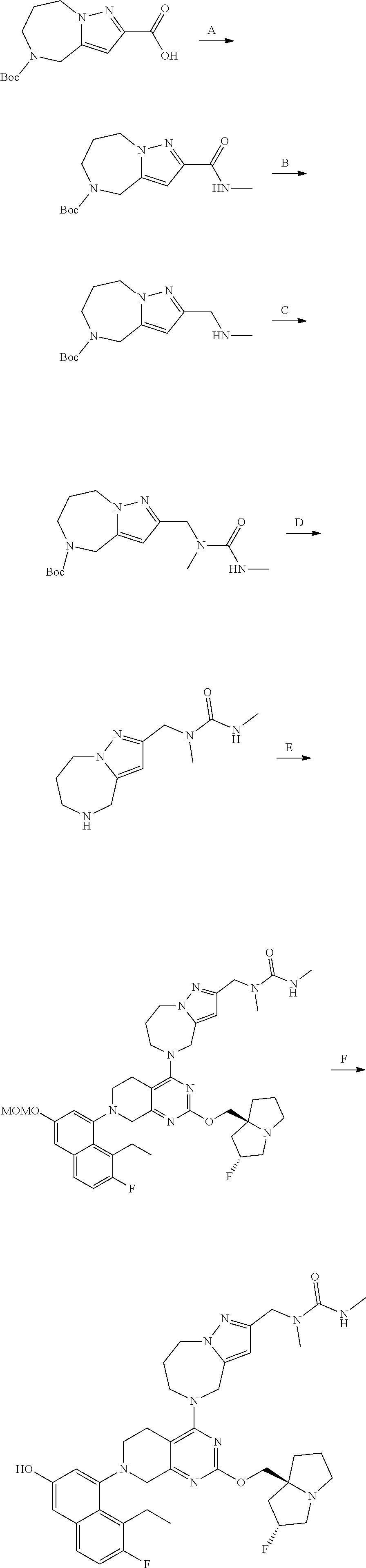

Step A. tert-butyl 2-(methylcarbamoyl)-7,8-dihydro-4H-pyrazolo[1,5-a][1,4]diazepine-5(6H)-carboxylate: To a solution of 5-(tert-butoxycarbonyl)-5,6,7,8-tetrahydro-4H-pyrazolo[1,5-a][1,4]diazepine-2-carboxylic acid (20.0 g, 1.0 equiv) in DMF (20 mL) were added HATU (5.41 g, 2.0 equiv), methanamine (960 mg, 2.0 equiv, HCl) and N,N-diethylpropan-2-amine (2.76 g, 3.0 equiv). The reaction was stirred at 20° C. for 2 hours. The mixture was filtered and purified by reversed phase HPLC (0.1% FA condition) to afford the title compound (1.28 mg, 59% yield) as white solid; LCMS (ESI, M+1): m/z=295.1.

Step B. tert-butyl 2-((methylamino)methyl)-7,8-dihydro-4H-pyrazolo[1,5-a][1,4]diazepine-5(6H)-carboxylate: To a solution of tert-butyl 2-(methylcarbamoyl)-7,8-dihydro-4H-pyrazolo[1,5-a][1,4]diazepine-5(6H)-carboxylate (1.00 g, 1.0 equiv) in THF (10 mL) was added BH3Me2S (10 M, 3.4 mL, 10 equiv) at 0° C. The reaction was stirred at 80° C. for 2 hours. The mixture was quenched with HCl (2 M) and stirred at 40° C. for 0.5 hours. The mixture was concentrated and purified by reversed phase HPLC (0.1% FA condition) to afford the title compound (270 mg, 25% yield) as white solid; LCMS (ESI, M+1): m/z=281.1.

Step C. tert-butyl 2-((1,3-dimethylureido)methyl)-7,8-dihydro-4H-pyrazolo[1,5-a][1,4]diazepine-5(6H)-carboxylate: To a solution of tert-butyl 2-((methylamino)methyl)-7,8-dihydro-4H-pyrazolo[1,5-a][1,4]diazepine-5(6H)-carboxylate (60.0 mg, 1.0 equiv) in DCM (0.5 mL) were added TEA (65.0 mg, 3.0 equiv) and methylcarbamic chloride (40.0 mg, 2.0 equiv). The reaction was stirred at 20° C. for 10 minutes. The mixture was concentrated and purified by reversed phase HPLC (0.1% FA condition) to afford the title compound (50.0 mg, 65% yield) as white solid; LCMS (ESI, M+1): m/z=338.0.

Step D. 1,3-dimethyl-1-((5,6,7,8-tetrahydro-4H-pyrazolo[1,5-a][1,4]diazepin-2-yll)methyll)urea: To a solution of tert-butyl 2-((1,3-dimethylureido)methyl)-7,8-dihydro-4H-pyrazolo[1,5-a][1,4]diazepine-5(6H)-carboxylate (40.0 mg, 1.0 equiv) in HCl•dioxane (4 M, 1.00 mL, 67 equiv) was stirred at 20° C. for 0.5 hours. The reaction was concentrated to afford the title compound (35.0 mg, crude, HCl) as white solid.

The last two steps were performed according to Example 248. The title compound was obtained as white solid (FA salt); 1H NMR (400 MHz, METHANOL-d4) 8=7.51 (dd, J=5.6, 8.8 Hz, 1H), 7.19-7.11 (m, 1H), 6.98-6.93 (m, 2H), 6.11 (s, 1H), 5.39-5.20 (m, 1H), 5.02-4.92 (m, 1H), 4.73 (br d, J=4.8 Hz, 1H), 4.48-4.39 (m, 3H), 4.37-4.29 (m, 1H), 4.20-4.12 (m, 2H), 4.11-4.04 (m, 2H), 4.01-3.90 (m, 1H), 3.73-3.65 (m, 1H), 3.54-3.46 (m, 1H), 3.43-3.36 (m, 2H), 3.24 (br s, 2H), 3.18 (br d, J=8.4 Hz, 2H), 3.05 (td, J=4.4, 9.6 Hz, 1H), 2.84-2.73 (m, 6H), 2.71 (br d, J=11.6 Hz, 1H), 2.32-2.23 (m, 2H), 2.23-2.17 (m, 1H), 2.14-1.94 (m, 5H), 1.92-1.84 (m, 1H), 1.15-1.09 (m, 3H); LCMS (ESI, M+1): m/z=716.6.

### Example 310

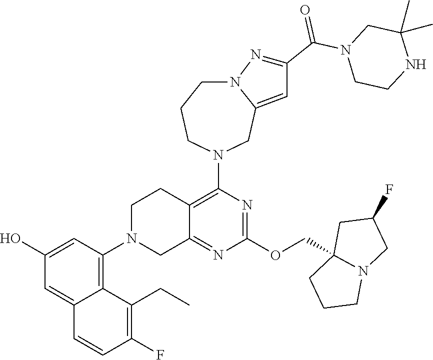 [SMILES: CCc1c(F)ccc2cc(O)cc(N3CCc4c(nc(OC[C@@]56CCCN5C[C@H](F)C6)nc4N4CCCn5nc(C(=O)N6CCNC(C)(C)C6)cc5C4)C3)c12]

### (3,3-dimethylpiperazin-1-yl)(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

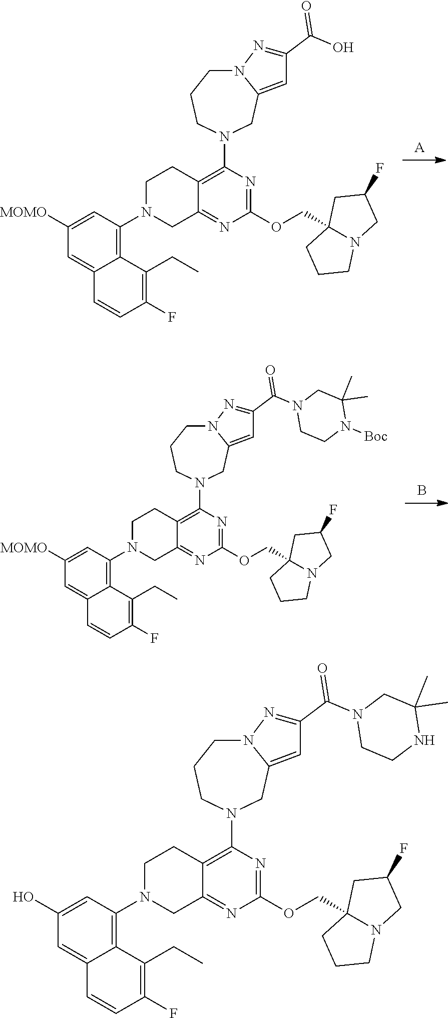

Synthesized according to Example 233 except that HCl instead of TFA was used in the last step. The title compound was obtained as orange solid. 1H NMR (400 MHz, dimethylsulfoxide-d6) δ=9.69 (s, 1H), 7.69-7.53 (m, 1H), 7.24 (t, J=9.2 Hz, 1H), 6.99 (s, 2H), 6.61-6.32 (m, 1H), 5.33-5.11 (m, 1H), 5.09-4.91 (m, 1H), 4.74 (br d, J=16.4 Hz, 1H), 4.60-4.38 (m, 2H), 4.16 (br d, J=2.4 Hz, 1H), 3.94-3.73 (m, 5H), 3.65-3.51 (m, 2H), 3.44 (br d, J=12.0 Hz, 2H), 3.28-3.14 (m, 3H), 3.13-3.02 (m, 3H), 2.97 (br s, 1H), 2.86-2.77 (m, 1H), 2.73 (br t, J=4.8 Hz, 2H), 2.63 (br s, 1H), 2.26-2.02 (m, 3H), 1.98-1.89 (m, 3H), 1.84-1.68 (m, 3H), 1.06 (br t, J=6.4 Hz, 3H), 1.01 (br s, 3H), 0.92 (br d, J=4.8 Hz, 3H); 19F NMR (376 MHz, dimethylsulfoxide-d6) δ=−121.333, -171.977;LCMS (ESI, M+1): m/z=756.7.

### Example 311

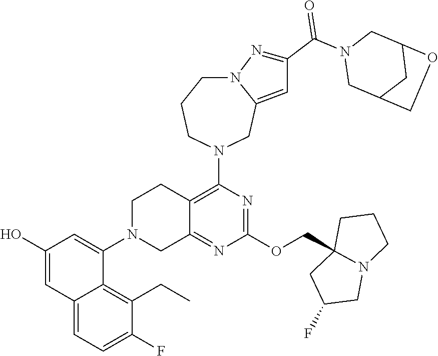 [SMILES: CCc1c(F)ccc2cc(O)cc(N3CCc4c(nc(OC[C@@]56CCCN5C[C@H](F)C6)nc4N4CCCn5nc(C(=O)N6CC7COC(C7)C6)cc5C4)C3)c12]

### 6-oxa-3-azabicyclo[3.2.1]octan-3-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

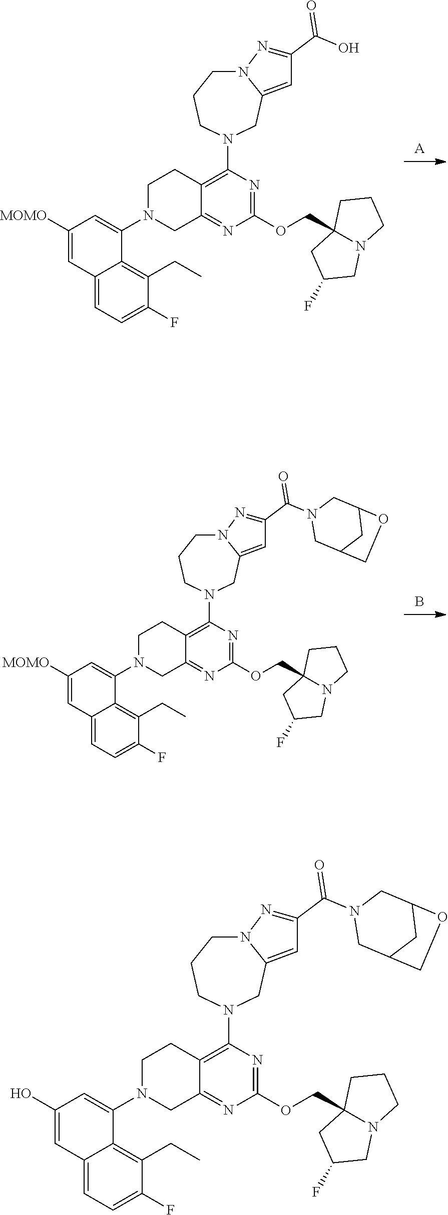

Synthesized according to Example 233 except that HCl instead of TFA was used in the last step. The title compound was obtained as white solid (FA salt). 1H NMR (400 MHz, METHANOL-d4) δ=7.53 (dd, J=5.6, 8.8 Hz, 1H), 7.16 (t, J=9.2 Hz, 1H), 6.99 (s, 2H), 6.64-6.57 (m, 1H), 5.44-5.22 (m, 1H), 5.11-4.90 (m, 2H), 4.68-4.31 (m, 4H), 4.24-3.78 (m, 7H), 3.69 (br d, J=17.6 Hz, 2H), 3.56 (br d, J=9.2 Hz, 1H), 3.48-3.34 (m, 6H), 3.30-2.86 (m, 5H), 2.67-2.50 (m, 1H), 2.41-2.22 (m, 4H), 2,21-1.91 (m, 6H), 1,19-1.06 (m, 3H); LCMS (ESI, M+1): m/z =755.6.

### Example 312

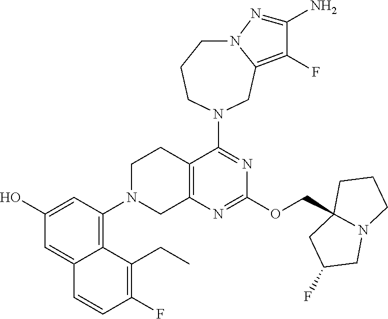 [SMILES: CCc1c(F)ccc2cc(O)cc(N3CCc4c(nc(OC[C@@]56CCCN5C[C@H](F)C6)nc4N4CCCn5nc(N)c(F)c5C4)C3)c12]

### 4-(4-(2-amino-3-fluoro-7,8-dihydro-4H-pyrazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

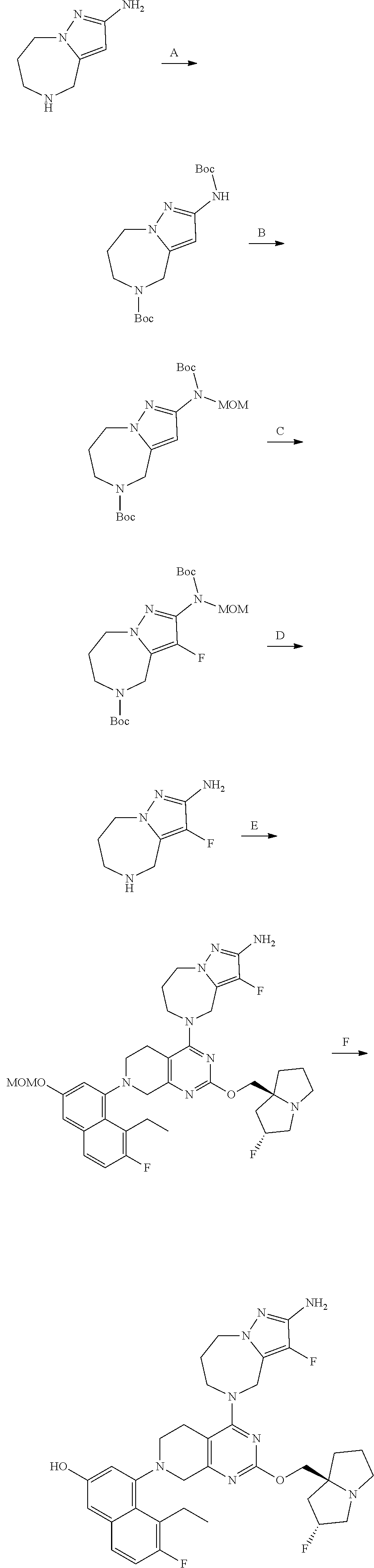

Step A. tert-butyl 2-((tert-butoxycarbonyl)amino)-7,8-dihydro-4H-pyrazolo[1,5-a][1,4]diazepine-5(6H)-carboxylate: 5,6,7,8-tetrahydro-4H-pyrazolo[1,5-a][1,4]diazepin-2-amine (1.00 g, 1.0 equiv) was dissolved into (Boc)20 (10 mL), and the mixture was stirred at 60° C. for 2 hours. The mixture was quenched with water (10 mL) and extracted with ethyl acetate (2×8 mL). The combined organic layers were dried over anhydrous sodium sulfate, concentrated, and purified by column chromatography (SiO2, petroleum ether/ethyl acetate=10/1 to 1/1) to afford the title compound (2.20 g, 93% yield) as a white solid; LCMS (ESI, M+1, M-55): m/z=353.2, 297.2.

Step B. tert-butyl 2-((tert-butoxycarbonyl)(methoxymethyl)amino)-7,8-dihydro-4H-pyrazolo[1,5-a][1,4]diazepine-5(6H)-carboxylate: To a solution of tert-butyl 2-((tert-butoxycarbonyl)amino)-7,8-dihydro-4H-pyrazolo[1,5-a][1,4]diazepine-5(6H)-carboxylate (2.00 g, 1.0 equiv) in THE (50 mL) was added NaH (454 mg, 60% purity, 2.0 equiv) in portions slowly at 0° C. The mixture was stirred at 0° C. for 30 minutes, and then bromo(methoxy)methane (1.06 g, 1.5 equiv) was added dropwise slowly at 0° C. The mixture was stirred at 25° C. for 30 minutes. The mixture was quenched with water (50 mL) and extracted with ethyl acetate (2×50 mL). The combined organic layers were dried over anhydrous sodium sulfate, concentrated, and purified by column chromatography (SiO2, petroleum ether/ethyl acetate=5/1 to 1/1) to afford the title compound (1.60 g, 67% yield) as a white solid; 1H NMR (400 MHz, CHLOROFORM-d) δ=6.36-6.08 (m, 1H), 5.15 (s, 2H), 4.38 (br s, 2H), 4.32-4.28 (m, 2H), 3.68 (br s, 2H), 3.41 (s, 3H), 1.92 (br s, 2H), 1.51 (s, 9H), 1.43 (s, 9H). LCMS (ESI, M+1, M-55): m/z=397.2, 341.2.

Step C. tert-butyl 2-((tert-butoxycarbonyl)(methoxymethyl)amino)-3-fluoro-7,8-dihydro-4H-pyrazolo[1,5-a][1,4]diazepine-5(6H)-carboxylate: To a solution of tert-butyl 2-((tert-butoxycarbonyl)(methoxymethyl)amino)-7,8-dihydro-4H-pyrazolo[1,5-a][1,4]diazepine-5(6H)-carboxylate (1.50 g, 1.0 equiv) in acetonitrile (5 mL) was added Select F (1.34 g, 1.0 equiv). The mixture was stirred at 40° C. for 2 hours. The reaction was filtered and washed with methanol (50 mL). The filtrate was concentrated under reduced pressure to dryness. The mixture was purified by prep-HPLC (column: Phenomenex luna C18 150×40 mm×15 μm; A: [water(FA)];B:ACN, B %: 45%-75%,10 min) and (column: Phenomenex luna C18 150×40 mm×15 m; mobile phase: [water(FA)-ACN]; B %: 45%-75%,10 min)) to afford the title compound (600 mg, 38% yield) as a white solid. LCMS (ESI, M+23): m/z=437.2.

Step D. 3-fluoro-5,6,7,8-tetrahydro-4H-pyrazolo[1,5-a][1,4]diazepin-2-amine: To a solution of tert-butyl 2-((tert-butoxycarbonyl)(methoxymethyl)amino)-3-fluoro-7,8-dihydro-4H-pyrazolo[1,5-a][1,4]diazepine-5(6H)-carboxylate (500 mg, 1.0 equiv) in methanol (12.5 mL) was added HCl•MeOH (4 M, 12.5 mL). The mixture was stirred at 0° C. for 0.5 hours. The reaction was concentrated under reduced pressure to dryness. The residue was dissolved in methanol (3 mL) and adjusted to pH=8 with NaHCO₃. The mixture was diluted with methanol (2 mL) for 0.5 hours and filtered. The filtrate was concentrated under reduced pressure to dryness. The residue was diluted with dichloromethane/methanol (10/1, 3 mL) and filtered. The filtrate was concentrated under reduced pressure to afford the title compound (140 mg, 68% yield) as a white solid.

The last two steps were performed according to Example 248. The title compound was obtained as a white solid. 1H NMR (400 MHz, METHANOL-d4) δ=7.51 (dd, J=5.6, 9.2 Hz, 1H), 7.14 (t, J=9.2 Hz, 1H), 7.02-6.92 (m, 2H), 5.34 (br s, 1H), 4.80-4.60 (m, 1H), 4.24-4.16 (m, 2H), 4.12-3.96 (m, 4H), 3.66 (br d, J=17.2 Hz, 1H), 3.56-3.44 (m, 2H), 3.44-3.36 (m, 3H), 3.24-3.12 (m, 4H), 3.04-2.92 (m, 1H), 2.72 (br d, J=14.0 Hz, 1H), 2.332-2.16 (m, 3H), 2.12-2.04 (m, 2H), 2.04-1.76 (m, 4H), 1.11 (t, J=7.2 Hz, 3H) LCMS (ESI, M+1): m/z=649.4.

### Example 313

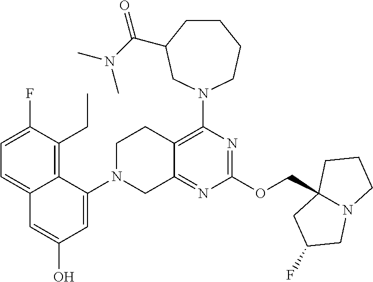 [SMILES: CCc1c(F)ccc2cc(O)cc(N3CCc4c(nc(OC[C@@]56CCCN5C[C@H](F)C6)nc4N4CCCCC(C(=O)N(C)C)C4)C3)c12]

### 1-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethylazepane-3-carboxamide

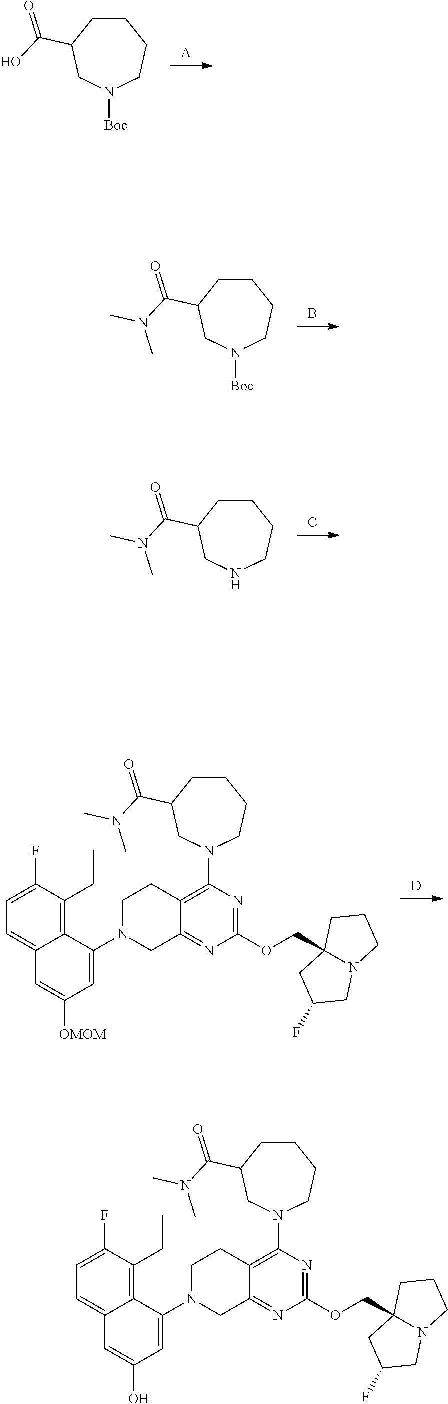

Step A. tert-butyl 3-(dimethylcarbamoyl)azepane-1-carboxylate: To a solution of 1-(tert-butoxycarbonyl)azepane-3-carboxylic acid (100 mg, 1.0 equiv) in DCM (0.1 mL) were added HATU (234 mg, 1.5 equiv), N,N-diethylpropan-2-amine (159 mg, 3.0 equiv) and N-methylmethanamine (50.2 mg, 1.5 equiv). The mixture was stirred at 20° C. for 12 hours. The reaction was concentrated and purified with flash silica gel chouromatography [ISCO®; 4 g SepaFlash® Silica Flash Column, Eluent of 20˜80% Ethyl acetate/Petroleum ether gradient @15 mL/minutes] to afford the title compound (105 mg, 95% yield) as yellow gum. 1H NMR (400 MHz, CHLOROFORM-d) δ=4.02-3.75 (m, 2H), 3.16-3.08 (m, 3H), 3.06-2.96 (m, 2H), 2.95-2.90 (m, 3H), 1.95-1.79 (m, 2H), 1.78-1.57 (m, 3H), 1.47 (d, J=2.0 Hz, 9H), 1.44-1.32 (m, 1H).

Step B. N,N-dimethylazepane-3-carboxamide: To a solution of tert-butyl 3-(dimethylcarbamoyl)azepane-1-carboxylate (100 mg, 1.0 equiv) in dioxane (0.5 mL) was added HCl/dioxane (0.5 mL, 4.0 M, 5.4 equiv). The mixture was stirred at 20° C. for 12 hours. The reaction was concentrated to afford the title compound (60 mg, crude) as yellow gum. 1H NMR (400 MHz, METHANOL-d4) δ=3.47-3.32 (m, 3H), 3.30-3.23 (m, 1H), 3.19-3.13 (m, 1H), 3.11 (s, 3H), 2.95 (s, 3H), 2.18-2.06 (m, 1H), 2.05-1.93 (m, 1H), 1.93-1.81 (m, 2H), 1.80-1.65 (m, 2H).

The last two steps were performed according to Example 248. The title compound was obtained as yellow solid (FA salt). 1H NMR (400 MHz, METHANOL-d4) δ=8.50 (br s, 1H), 7.56-7.44 (m, 1H), 7.35 (br d, J=8.0 Hz, 1H), 7.28-7.22 (m, 1H), 7.14 (t, J=9.2 Hz, 1H), 7.03-6.91 (m, 2H), 5.51-5.27 (m, 1H), 4.45-3.97 (m, 5H), 3.78-3.59 (m, 2H), 3.57-3.43 (m, 5H), 3.42-3.33 (m, 2H), 3.23 (br d, J=3.2 Hz, 1H), 3.19 (s, 3H), 3.16-3.07 (m, 2H), 2.96 (s, 3H), 2.81-2.69 (m, 1H), 2.49-2.25 (m, 2H), 2.23-2.06 (m, 3H), 2.02-1.83 (m, 4H), 1.83-1.62 (m, 2H), 1.60-1.22 (m, 2H), 1.09 (q, J=6.4 Hz, 3H); LCMS (ESI, M+1): m/z=649.4.

### Example 314

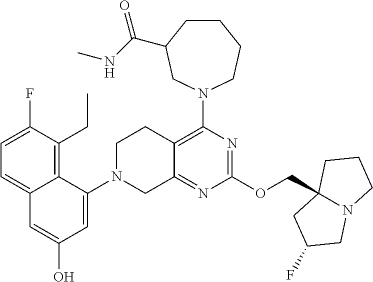 [SMILES: CCc1c(F)ccc2cc(O)cc(N3CCc4c(nc(OC[C@@]56CCCN5C[C@H](F)C6)nc4N4CCCCC(C(=O)NC)C4)C3)c12]

### 1-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N-methylazepane-3-carboxamide

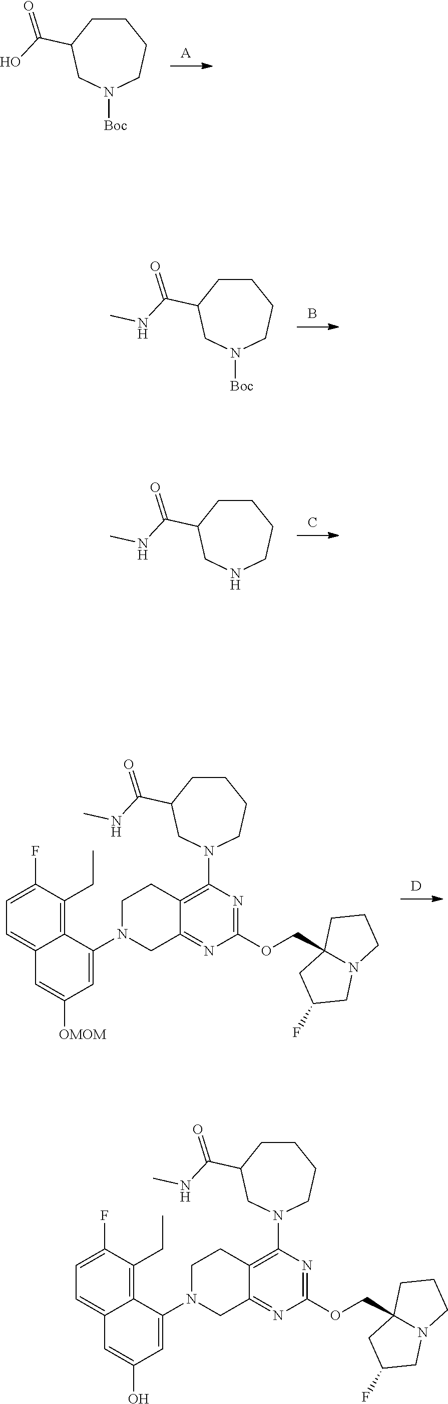

Step A. tert-butyl 3-(methylcarbamoyl)azepane-1-carboxylate: To a solution of 1-(tert-butoxycarbonyl)azepane-3-carboxylic acid (100 mg, 1.0 equiv) in DCM (1.0 mL) were added HATU (234 mg, 1.5 equiv), N,N-diethylpropan-2-amine (159 mg, 3.0 equiv) and methanamine (36.1 mg, 1.3 equiv). The mixture was stirred at 20° C. for 12 hours. The reaction was concentrated and purified with flash silica gel chouromatography [ISCO®; 4 g SepaFlash® Silica Flash Column, Eluent of 0-100% Ethyl acetate/Petroleum ether; gradient: 15 mL/minutes] to afford the title compound (100 mg, 76% yield) as yellow oil. 1H NMR (400 MHz, CHLOROFORM-d) δ=7.07 (br d, J=3.6 Hz, 1H), 3.64-3.45 (m, 2H), 3.25 (ddd, J=5.2, 9.2, 14.0 Hz, 1H), 2.78 (br s, 3H), 2.67-2.55 (m, 1H), 1.98-1.80 (m, 2H), 1.79-1.54 (m, 3H), 1.47 (s, 9H), 1.42-1.34 (m, 1H).

Step B. N-methylazepane-3-carboxamide: To a solution of tert-butyl 3-(methylcarbamoyl)azepane-1-carboxylate (100 mg, 1.0 equiv) in dioxane (0.5 mL) was added HCl/dioxane (0.5 mL, 4.0 M, 5.4 equiv). The mixture was stirred at 20° C. for 12 hours. The reaction was concentrated to afford the title compound (60 mg, crude) as yellow gum. 1H NMR (400 MHz, METHANOL-d4) δ=3.46-3.38 (m, 1H), 3.28 (br d, J=4.4 Hz, 1H), 3.15 (ddd, J=4.0, 8.8, 13.2 Hz, 1H), 2.86 (s, 5H), 2.84-2.79 (m, 1H), 2.12-2.00 (m, 1H), 1.98-1.78 (m, 4H), 1.77-1.64 (m, 1H).

The last two steps were performed according to Example 248. The title compound was obtained as yellow solid. 1H NMR (400 MHz, METHANOL-d4) δ=8.53 (s, 1H), 7.60-7.40 (m, 1H), 7.14 (t, J=9.2 Hz, 1H), 7.06-6.87 (m, 2H), 5.42-5.25 (m, 1H), 4.68-4.62 (m, 1H), 4.45-4.24 (m, 1H), 4.24-3.89 (m, 4H), 3.78-3.64 (m, 1H), 3.64-3.57 (m, 3H), 3.56-3.35 (m, 5H), 3.28-2.92 (m, 4H), 2.76-2.73 (m, 3H), 2.44-2.17 (m, 2H), 2.15-1.99 (m, 3H), 1.99-1.84 (m, 4H), 1.73 (q, J=12.0 Hz, 2H), 1.55-1.33 (m, 1H), 1.18 (t, J=7.2 Hz, 3H), 1.14-1.05 (m, 3H); LCMS (ESI, M+1): m/z=635.4.

### Example 315

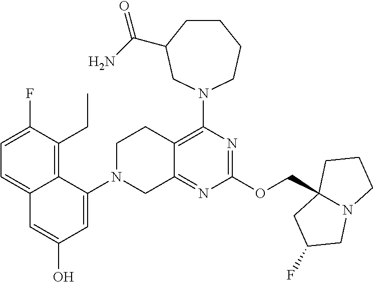 [SMILES: CCc1c(F)ccc2cc(O)cc(N3CCc4c(nc(OC[C@@]56CCCN5C[C@H](F)C6)nc4N4CCCCC(C(N)=O)C4)C3)c12]

### 1-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)azepane-3-carboxamide

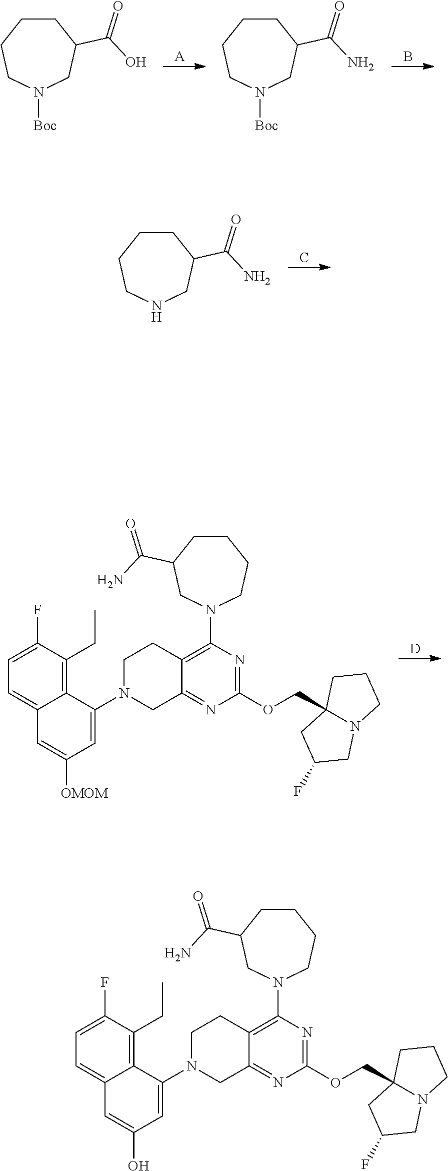

Step A. tert-butyl 3-carbamovlazepane-1-carboxylate. To a solution of 1-(tert-butoxycarbonyl)azepane-3-carboxylic acid (100 mg, 1.0 equiv) in DCM (1.0 mL) were added HA TU (234 mg, 1.5 equiv), N,N-diethylpropan-2-amine (159 mg, 3.0 equiv) and NH₄Cl (28.6 mg, 1.3 equiv). The mixture was stirred at 20° C. for 12 hours. The reaction was concentrated to afford the title compound (90 mg, 90% yield) as white solid. 1H NMR (400 MHz, DMSO-d6) 6S=13.80 (br s, 1H), 8.76 (dd, J=1.2, 4.4 Hz, 1H), 8.53 (dd, J=1.2, 8.4 Hz, 1H), 7.31 (br d, J=16.0 Hz, 1H), 6.76 (br s, 1H), 3.75-3.42 (m, 3H), 3.20-2.94 (m, 3H), 1.79-1.60 (m, 3H), 1.59-1.44 (m, 2H), 1.39 (s, 9H).

Step B. azepane-3-carboxamide: To a solution of tert-butyl 3-carbamoylazepane-1-carboxylate (90.0 mg, 1.0 equiv) in dioxane (0.5 mL) was added HCl/dioxane (0.5 mL, 4.0 M, 5.4 equiv). The mixture was stirred at 20° C. for 12 hours. The reaction was concentrated to afford the title compound (60 mg, crude) as yellow gum. 1H NMR (400 MHz, METHANOL-d4) 6S=8.50 (dd, J=1.2, 4.4 Hz, 1H), 8.16 (dd, J=1.2, 8.4 Hz, 1H), 7.31 (dd, J=4.4, 8.4 Hz, 1H), 3.07-3.00 (m, 4H), 2.91 (d, J=8.4 Hz, 2H), 2.60-2.51 (m, 4H).

The last two steps were performed according to Example 248. The title compound was obtained as brown solid. 1H NMR (400 MHz, METHANOL-d4) δ=7.59-7.49 (m, 1H), 7.18 (t, J=9.2 Hz, 1H), 7.11-6.99 (m, 2H), 5.69-5.47 (m, 1H), 4.72-4.60 (m, 1H), 4.52-4.38 (m, 1H), 4.33-4.09 (m, 2H), 4.06-3.82 (m, 5H), 3.80 (br s, 1H), 3.59-3.33 (m, 5H), 3.29-2.73 (m, 4H), 2.72-2.50 (m, 2H), 2.48-2.17 (m, 4H), 2.10-1.93 (m, 4H), 1.81-1.44 (m, 2H), 1.23-1.08 (m, 3H); LCMS (ESI, M+1): m/z=621.4.

### Example 316

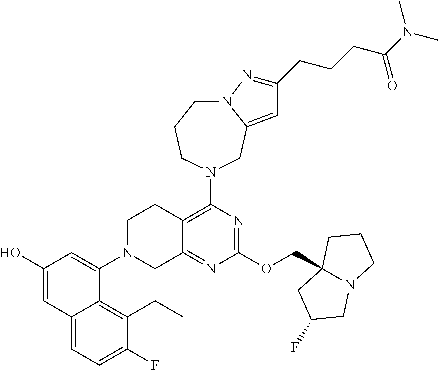 [SMILES: CCc1c(F)ccc2cc(O)cc(N3CCc4c(nc(OC[C@@]56CCCN5C[C@H](F)C6)nc4N4CCCn5nc(CCCC(=O)N(C)C)cc5C4)C3)c12]

### 4-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N,N-dimethylbutanamide

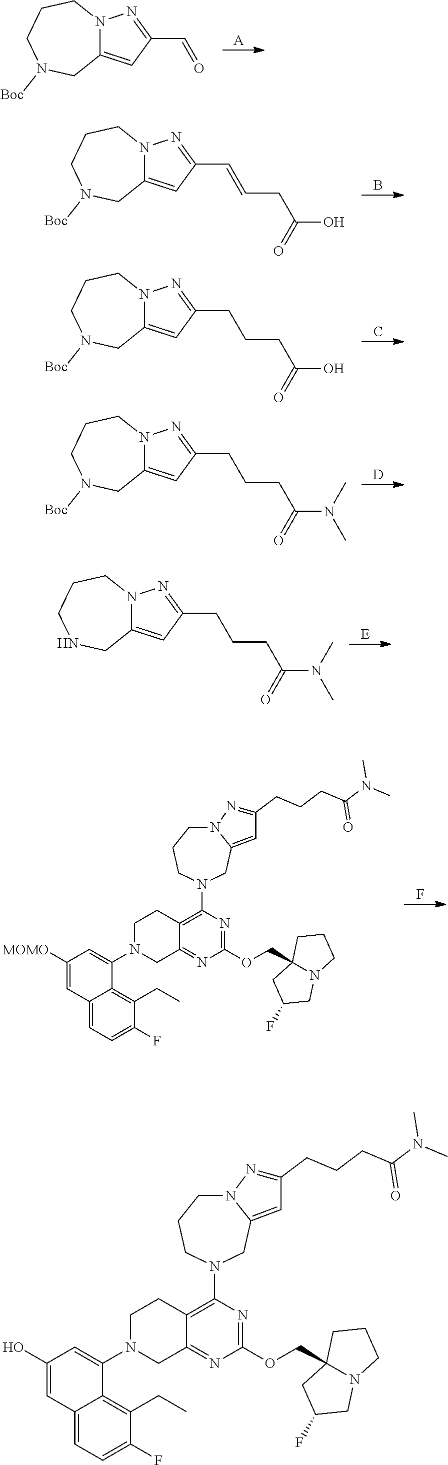

Step A. (E)-4-(5-(tert-butoxycarbonyl)-5,6,7,8-tetrahydro-4H-pyrazolo[1,5-a][1,4]diazepin-2-yl)but-3-enoic acid. A mixture of tert-butyl 2-formyl-7,8-dihydro-4H-pyrazolo[1,5-a][1,4]diazepine-5(6H)-carboxylate (500 mg, 1.0 equiv) and 2-carboxyethyl(triphenyl)phosphonium;bromide (860 mg, 1.1 equiv) in THF (12 mL) was added t-BuOK (3.77 mL, 1.0 M, 2.0 equiv) at 0° C. for 1 hour. The mixture was degassed and purged with N2 3 times, and then stirred at 20° C. for 16 hours under N2 atmosphere. The reaction was concentrated and purified with prep-HPLC [column: Phenomenex luna C18 150×40 mm×15 pm; mobile phase: [water (FA)-ACN]; B %: 23%-53%, 10 minutes] to afford the title compound (100 mg, 17% yield) as white solid. LCMS (ESI, M+1): m/z=322.2.

Step B. 4-(5-(tert-butoxycarbonyl)-5,6,7,8-tetrahydro-4H-pyrazolo[1,5-a][1,4]diazepin-2-yl)butanoic acid: To a solution of (E)-4-(5-(tert-butoxycarbonyl)-5,6,7,8-tetrahydro-4H-pyrazolo[1,5-a][1,4]diazepin-2-yl)but-3-enoic acid (100 mg, 1.0 equiv) in EtOH (4.0 mL) was added Pd/C (30 mg) under N2 atmosphere. The mixture was degassed and purged with H2 3 times. The mixture was stirred under H2 (15 Psi) at 20° C. for 1 hour. The reaction was filtered and concentrated to afford the title compound (100 mg, crude) as yellow oil. LCMS (ESI, M+1): m/z=324.1

Step C. tert-butyl 2-(4-(dimethylamino)-4-oxobutyl)-7,8-dihydro-4H-pyrazolo[1,5-a][1,4]diazepine-5(6H)-carboxylate: To a solution of 4-(5-(tert-butoxycarbonyl)-5,6,7,8-tetrahydro-4H-pyrazolo[1,5-a][1,4]diazepin-2-yl)butanoic acid (100 mg, 1.0 equiv) in THF (2.0 mL) were added HATU (176 mg, 1.5 equiv), N,N-diethylpropan-2-amine (120 mg, 3.0 equiv) and (CH₃)₂NH (309 μL, 2.0 M, 2.0 equiv). The mixture was stirred at 20° C. for 1 hour. The reaction was diluted with water (20 mL) and extracted with DCM (3×20 mL). The combined organic layers were washed with brine (20 mL), dried over Na₂SO₄, and concentrated to afford the title compound (100 mg, crude) as yellow oil. LCMS (ESI, M+1): m/z=351.2.

Step D. N,N-dimethyl-4-(5,6,7,8-tetrahydro-4H-pyrazolo[1,5-a][1,4]diazepin-2-1llbutanamide: To a solution of tert-butyl 2-(4-(dimethylamino)-4-oxobutyl)-7,8-dihydro-4H-pyrazolo[1,5-a][1,4]diazepine-5(6H)-carboxylate (100 mg, 1.0 equiv) was added HCl/MeOH (2.0 mL, 4.0 M). The mixture was stirred at 20° C. for 1 hour. The reaction was concentrated and purified with prep-HPLC [column: Waters Xbridge 150×25 mm×5 μm; mobile phase: [water (NH₄HCO₃)-ACN]; B %: 1%-30%, 10 minutes] to afford the title compound (50.0 mg, 70% yield) as white solid. LCMS (ESI, M+1): m/z=251.2.

The last two steps were performed according to Example 248. The title compound was obtained as white solid (FA salt). 1H NMR (400 MHz, METHANOL-d4) δ=8.64-8.42 (m, 1H), 7.51 (dd, J=5.8, 9.2 Hz, 1H), 7.15 (t, J=9.2 Hz, 1H), 6.98-6.91 (m, 2H), 6.07 (s, 1H), 5.44-5.25 (m, 1H), 4.80-4.72 (m, 2H), 4.41 (br d, J=4.4 Hz, 2H), 4.26-4.13 (m, 3H), 4.08 (br d, J=17.6 Hz, 1H), 4.00-3.89 (m, 1H), 3.70 (br d, J=17.6 Hz, 1H), 3.55-3.48 (m, 1H), 3.47-3.33 (m, 5H), 3.18 (br d, J=8.8 Hz, 2H), 2.99 (d, J=1.6 Hz, 3H), 2.90 (s, 3H), 2.72 (br s, 1H), 2.62-2.55 (m, 2H), 2.45-2.05 (m, 9H), 1.95-1.84 (m, 3H), 1.11 (br t, J=6.8 Hz, 3H); LCMS (ESI, M+1): m/z=729.5.

### Example 317

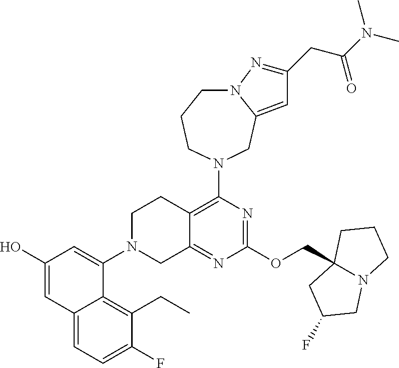 [SMILES: CCc1c(F)ccc2cc(O)cc(N3CCc4c(nc(OC[C@@]56CCCN5C[C@H](F)C6)nc4N4CCCn5nc(CC(=O)N(C)C)cc5C4)C3)c12]

### 2-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N,N-dimethylacetamide

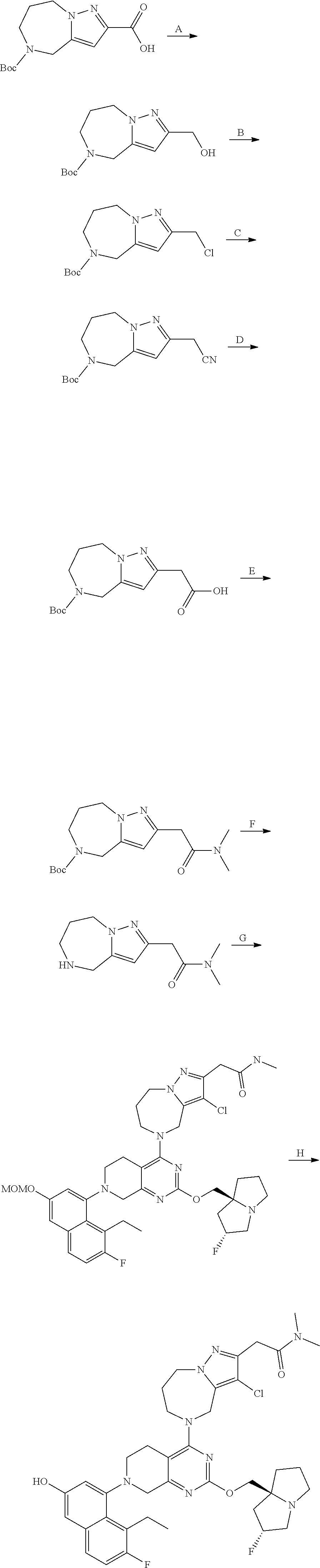

Step A. tert-butyl 2-(hydroxymethyl)-7,8-dihydro-4H-pyrazolo[1,5-a][1,4]diazepine-5(6H)-carboxylate: A mixture of 5-(tert-butoxycarbonyl)-5,6,7,8-tetrahydro-4H-pyrazolo[1,5-a][1,4]diazepine-2-carboxylic acid (8.5 g, 1.0 equiv) in THE (150 mL) was added BH3.THF (121 mL, 1.0 M, 4.0 equiv) at 0° C. under N2. The mixture was stirred at 60° C. for 12 hours under N2 atmosphere. The reaction was quenched by MeOH (200 mL) at 0° C., concentrated, and purified with prep-HPLC [Column: 120 g Flash Column WelchUltimate XB_C18 20-40 μm; 120 A; Mobile phase: MeCN/H₂O; Flow rate: 85 mL/minute; Gradient B %: 5-40%, 20 minutes; 40% 5 minutes] to afford the title compound (5.7 g, 71% yield) as colorless oil. 1H NMR (400 MHz, DMSO-d6) δ=8.13 (s, 1H), 6.06 (s, 1H), 4.92 (br s, 1H), 4.50-4.23 (m, 6H), 3.62 (br s, 2H), 1.71 (br s, 2H), 1.34 (br s, 9H); LCMS (ESI, M+1): m/z=268.2.

Step B. tert-butyl 2-(chloromethyl)-7,8-dihydro-4H-pyrazolo[1,5-a][1,4]diazepine-5(6H)-carboxylate: A mixture of tert-butyl 2-(hydroxymethyl)-7,8-dihydro-4H-pyrazolo[1,5-a][1,4]diazepine-5(6H)-carboxylate (2.50 g, 1.0 equiv), MsCl (4.05 g, 3.8 equiv) and TEA (2.84 g, 3.0 equiv) in DCM (30 mL) was degassed and purged with N2 3 times at 0° C. The mixture was stirred at 20° C. for 3 hours under N2 atmosphere. The reaction was quenched by NaHCO₃ solution (50 mL) at 0° C., and then extracted with EtOAc (3×50 mL). The combined organic layers were washed with brine (50 mL), dried over Na₂SO₄, and concentrated to afford the title compound (2.80 g, crude) as yellow oil. LCMS (ESI, M+1): m/z=286.1.

Step C. tert-butyl 2-(cyanomethyl)-7,8-dihydro-4H-pyrazolo[1,5-a][1,4]diazepine-5(6H)-carboxylate: To a solution of tert-butyl 2-(chloromethyl)-7,8-dihydro-4H-pyrazolo[1,5-a][1,4]diazepine-5(6H)-carboxylate (2.80 g, 1.0 equiv) in DMF (30 mL) was added NaCN (1.55 g, 3.2 equiv). The mixture was stirred at 60° C. for 3 hours. The reaction was diluted with water (200 mL) at 0° C. and extracted with EtOAc (3×200 mL). The combined organic layers were washed with brine (200 mL), dried over Na₂SO₄, concentrated, and purified with column chromatography [SiO₂, Petroleum ether/Ethyl acetate=5/1 to 0/1] to afford the title compound (1.40 g, 52% yield) as yellow solid. 1H NMR (400 MHz, DMSO-d6) δ=6.12 (s, 1H), 4.42 (s, 2H), 4.38-4.31 (m, 2H), 3.88 (s, 2H), 3.62 (br s, 2H), 1.72 (br s, 2H), 1.33 (br s, 9H); LCMS (ESI, M+1): m/z=277.1.

Step D. 2-(5-(tert-butoxycarbonyl)-5,6,7,8-tetrahydro-4H-pyrazolo[1,5-a][1,4]diazepin-2-yl)acetic acid: To a solution of tert-butyl 2-(cyanomethyl)-7,8-dihydro-4H-pyrazolo[1,5-a][1,4]diazepine-5(6H)-carboxylate (700 mg, 1.0 equiv) in EtOH (15 mL) and water (3 mL) was added NaOH (203 mg, 2.0 equiv). The mixture was stirred at 90° C. for 3 hours. The reaction was then diluted with water (30 mL) and extracted with EtOAc (3×30 mL). The combined organic layers were washed with brine (30 mL), dried over Na₂SO₄, and concentrated to afford the title compound (700 mg, 94% yield) as yellow solid. LCMS (ESI, M+1): m/z=296.1.

Step E. tert-butyl 2-(2-(dimethylamino)-2-oxoethyl)-7,8-dihydro-4H-pyrazolo[1,5-a][1,4]diazepine-5(6H)-carboxylate: To a solution of 2-(5-(tert-butoxycarbonyl)-5,6,7,8-tetrahydro-4H-pyrazolo[1,5-a][1,4]diazepin-2-yl)acetic acid (200 mg, 1.0 equiv) in THE (6 mL) were added N,N-diethylpropan-2-amine (262 mg, 3.0 equiv), HATU (386 mg, 1.5 equiv) and (CH₃)₂NH (677 L, 2 M, 2.0 equiv) The mixture was stirred at 20° C. for 2 hours. The reaction was diluted with water (30 mL) and extracted with DCM (3×30 mL). The combined organic layers were washed with brine (30 mL), dried over Na₂SO₄, and concentrated to afford the title compound (200 mg, crude) as yellow oil. LCMS (ESI, M+1): m/z=323.2.

Step F. N,N-dimethyl-2-(5,6,7,8-tetrahydro-4H-pyrazolo[1,5-a][1,4]diazepin-2-yl)acetamide: To a solution of tert-butyl 2-(2-(dimethylamino)-2-oxoethyl)-7,8-dihydro-4H-pyrazolo[1,5-a][1,4]diazepine-5(6H)-carboxylate (200 mg, 1.0 equiv) in MeOH (3.0 mL) was added HCl/MeOH (3.0 mL, 4.0 M, 19 equiv). The mixture was stirred at 20° C. for 1 hour. The reaction was concentrated to afford the title compound (200 mg, crude) as yellow oil. LCMS (ESI, M+1): m/z=223.2.

The last two steps were performed according to Example 248. The title compound was obtained as white solid (FA salt). 1H NMR (400 MHz, METHANOL-d4) δ=7.52 (dd, J=6.0, 9.2 Hz, 1H), 7.15 (t, J=9.2 Hz, 1H), 6.99-6.91 (m, 2H), 6.13 (s, 1H), 5.46-5.26 (m, 1H), 4.83-4.77 (m, 2H), 4.47-4.39 (m, 2H), 4.28-4.20 (m, 1H), 4.20-4.12 (m, 2H), 4.11-3.98 (m, 2H), 3.75-3.63 (m, 3H), 3.55-3.36 (m, 6H), 3.23-3.13 (m, 3H), 3.10 (s, 3H), 2.95 (s, 3H), 2.78-2.63 (m, 1H), 2.45-2.17 (m, 4H), 2.14-1.94 (m, 4H), 1.14-1.05 (m, 3H); LCMS (ESI, M+1): m/z=701.4.

### Example 318

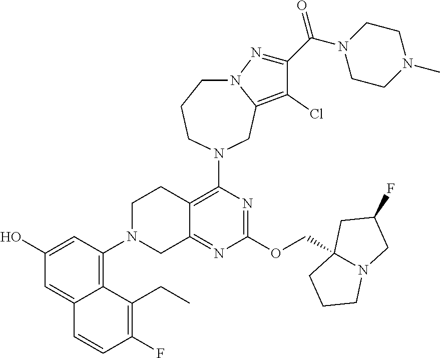 [SMILES: CCc1c(F)ccc2cc(O)cc(N3CCc4c(nc(OC[C@@]56CCCN5C[C@H](F)C6)nc4N4CCCn5nc(C(=O)N6CCN(C)CC6)c(Cl)c5C4)C3)c12]

### (3-chloro-5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-prazolo[1,5-a][1,4]diazepin-2-yl)(4-methylpiperazin-1-yl)methanone

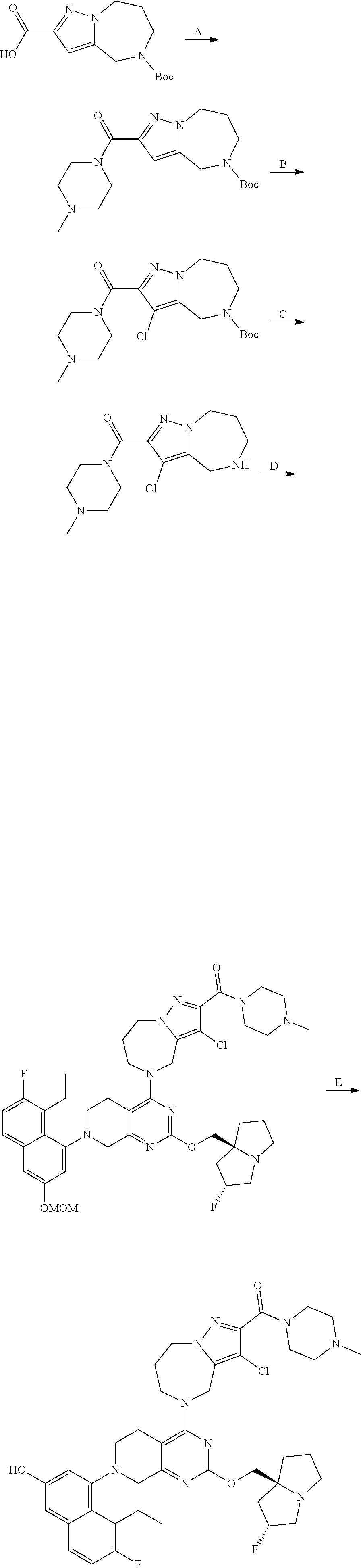

Step A tert-butyl 2-(4-methylpiperazine-1-carbonyl)-7,8-dihydro-4H-pyrazolo[1,5-a][1,4]diazepine-5(6H)-carboxylate: To a solution of 5-tert-butoxycarbonyl-4,6,7,8-tetrahydropyrazolo[1,5-a][1,4]diazepine-2-carboxylic acid (1.00 g, 1.0 equiv) in DMF (10.0 mL) was added 1-methylpiperazine (356 mg, 1.0 equiv), HATU (4.05 g, 3.0 equiv) and N,N-diethylpropan-2-amine (4.59 g, 10.0 equiv).The mixture was stirred at 20° C. for 1 hour. After, the reaction was diluted with water (100 mL) and extracted with ethyl acetate (100 mL×2). The combined organic layers were washed with brine (100 mL), dried over anhydrous sodium sulfate, filtered, concentrated, and purified with column chromatography [SiO2, dichloromethane/methanol=10/1) to afford the title compound (1.20 g, 90% yield, 97% purity) as a red solid. LCMS (ESI, M+1): m/z=364.3.

Step B: tert-butyl 3-chloro-2-(4-methylpiperazine-1-carbonyl)-7,8-dihydro-4H-pyrazolo[1,5-a][1,4]diazepine-5(6H)-carboxylate: To a solution of tert-butyl 2-(4-methylpiperazine-1-carbonyl)-4,6,7,8-tetrahydropyrazolo[1,5-a][1,4]diazepine-5-carboxylate (1.20 g, 1.0 equiv) in DMF (15.0 mL) was added NCS (553 mg, 1.3 equiv). The mixture was stirred at 20° C. for 24 hours. The reaction was diluted with water (200 mL) and extracted with ethyl acetate (200 mL×2). The combined organic layers were washed with brine (200 mL), dried over anhydrous sodium sulfate, filtered, concentrated, and purified with column chromatography (SiO2, dichloromethane/methanol=20/1 to 10/1) to afford the title compound (400 mg, 32% yield) as a yellow solid. 1H NMR (400 MHz, DMSO-d6) δ=4.53 (s, 2H), 4.46-4.39 (m, 2H), 3.67 (br s, 2H), 3.27-2.90 (m, 4H), 2.80-2.60 (m, 4H), 2.52 (br s, 3H), 1.82 (br s, 2H), 1.36 (s, 9H). LCMS (ESI, M+1): m/z=398.2.

Step C: (3-chloro-5,6,7,8-tetrahydro-4H-pyrazolo[1,5-a][1,4]diazepin-2-yl)(4-methylpiperazin-1-yl)methanone: A solution of tert-butyl 3-chloro-2-(4-methylpiperazine-1-carbonyl)-4,6,7,8-tetrahydropyrazolo[1,5-a][1,4]diazepine-5-carboxylate (400 mg, 1.0 equiv) in MeCN (4.0 mL) and HCl/dioxane (4 M, 4.00 mL, 15.9 equiv) was stirred at 20° C. for 1 hour. The reaction was concentrated to afford the title compound (500 mg, crude) as a colorless oil. LCMS (ESI, M+1): m/z=298.1.

The last two steps were performed according to Example 248. The title compound was obtained as yellow solid. 1H NMR (400 MHz, CD3OD) δ=7.51 (dd, J=6.0, 8.8 Hz, 1H), 7.15 (t, J=9.6 Hz, 1H), 7.00-6.93 (m, 2H), 5.37-5.19 (m, 1H), 5.05-4.89 (m, 2H), 4.83-4.56 (m, 2H), 4.55-4.38 (m, 2H), 4.15-3.95 (m, 5H), 3.80-3.62 (m, 5H), 3.56-3.36 (m, 4H), 3.25-3.12 (m, 4H), 3.05-2.96 (m, 1H), 2.71 (br d, J=15.2 Hz, 1H), 2.55-2.43 (m, 4H), 2.30-1.80 (m, 8H), 1.40-1.25 (m, 1H), 1.10 (t, J=6.8 Hz, 3H); LCMS (ESI, M+1): m/z=776.3.

### Example 319

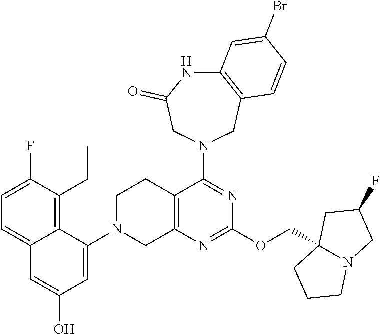 [SMILES: CCc1c(F)ccc2cc(O)cc(N3CCc4c(nc(OC[C@@]56CCCN5C[C@H](F)C6)nc4N4CC(=O)Nc5cc(Br)ccc5C4)C3)c12]

### 8-bromo-4-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,4,5-tetrahydro-2H-benzo[e][1,4]diazepin-2-one

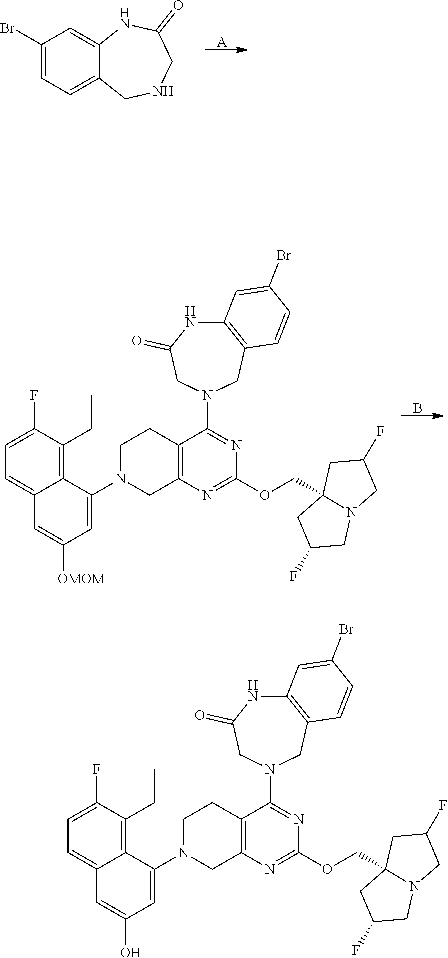

Synthesized according to Example 248 except that TFA instead of HCl was used in the last step. The title compound was obtained as white solid (TFA salt). 1H NMR (400 MHz, METHANOL-d4) δ=7.52 (dd, J=5.6, 8.8 Hz, 1H), 7.28-7.10 (m, 5H), 7.02-6.90 (m, 2H), 5.66-5.42 (m, 1H), 5.12-5.02 (m, 2H), 4.60-4.59 (m, 1H), 4.66 (d, J=5.6 Hz, 3H), 4.05-3.67 (m, 6H), 3.62-3.44 (m, 2H), 3.27-3.07 (m, 2H), 2.89-2.07 (m, 8H), 0.93 (q, J=7.6 Hz, 3H); 1 LCMS (ESI, M+1): m/z=721.2

### Example 320

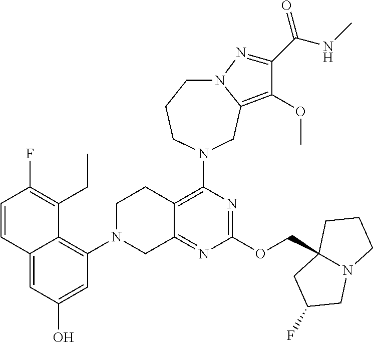 [SMILES: CCc1c(F)ccc2cc(O)cc(N3CCc4c(nc(OC[C@@]56CCCN5C[C@H](F)C6)nc4N4CCCn5nc(C(=O)NC)c(OC)c5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-3-methoxy-N-methyl-5,6,7,8-tetrahydro-4H-pyrazolo[1,5-a][1,4]diazepine-2-carboxamide

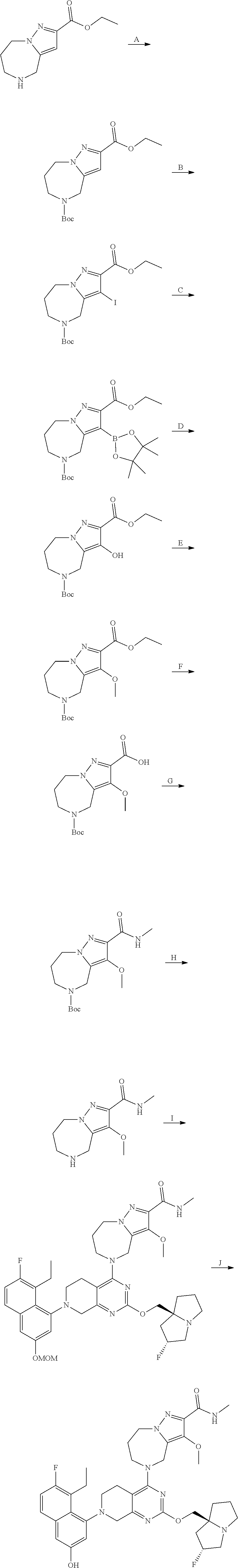

Step A. 5-tert-butyl 2-ethyl 7,8-dihydro-4H-pyrazolo[1,5-a][1,4]diazepine-2,5(6H)-dicarboxylate: To a solution of ethyl 5,6,7,8-tetrahydro-4H1-pyrazolo[1,5-a][1,4]diazepine-2-carb oxyl ate (8.40 g, 1.0 equiv, HCi) in DCM (100 mL) were added TEA (10.4 g, 3.0 equiv) and Boc20 (14.9 g, 2.0 equiv). The reaction was stirred at 25° C. for 1 hour. The mixture was diluted with water (50 mL) and extracted with DCM (2×30 mL). The combined organic layers were dried over anhydrous sodium sulfate, concentrated, and purified by column chromatography (SiO₂, petroleum ether/ethyl acetate=10/1 to 1/1) to afford the title compound (9.90 g, 92% yield) as yellow solid; LCMS (ESI, M+1): m/z=310.0.

Step B. 5-tert-butyl 2-ethyl 3-iodo-7,8-dihydro-4H-pyrazolo[1,5-a][1,4]diazepine-2,5(6H)-dicarboxylate: To a solution of 5-tert-butyl 2-ethyl 7,8-dihydro-4H-pyrazolo[1,5-a][1,4]diazepine-2,5(6H)-dicarboxylate (4.00 g, 1.0 equiv) in AcOH (40 mL) was added 1-iodopyrrolidine-2,5-dione (5.82 g, 2.0 equiv). The reaction was stirred at 80° C. for 1 hour. The reaction was neutralized with saturated NaHCO₃ aqueous solution (100 mL) and extracted with EtOAc (3×100 mL). The combined organic layers were washed with Na2SO3 (100 mL) and brine (100 mL), dried over anhydrous sodium sulfate, concentrated, and purified by reversed phase HPLC (0.1% FA condition) to afford the title compound (4.70 g, 82% yield) as yellow solid; LCMS (ESI, M+1): m/z=436.0.

Step C. 5-tert-butyl 2-ethyl 3-(4,4,5,5-tetramethyl-1,3,2-dioxaborolan-2-yl)-7,8-dihydro-4H-pyrazolo[1,5-a][1,4]diazepine-2,5(6H)-dicarboxylate: To a solution of 05-tert-butyl 02-ethyl 3-iodo-4,6,7,8-tetrahydropyrazolo[1,5-a][1,4]diazepine-2,5-dicarboxylate (1.80 g, 1.0 equiv) and 4,4,5,5-tetramethyl-1,3,2-dioxaborolane (5.29 g, 10 equiv) in MeCN (18 mL) were added bis(triphenylphosphine)palladium(II) chloride (303 mg, 0.10 equiv) and TEA (1.26 g, 3.0 equiv). The reaction was stirred at 40° C. for 12 hours. The mixture was filtered and purified by reversed-phase HPLC (0.1% FA condition) to afford the title compound (1.00 g, 50% yield) as white solid; LCMS (ESI, M-137): m/z=297.8.

Step D. 5-tert-butyl 2-ethyl 3-hydroxy-7,8-dihydro-4H-pyrazolo[1,5-a][1,4]diazepine-2,5(6H)-dicarboxylate: To a solution of 5-tert-butyl 2-ethyl 3-(4,4,5,5-tetramethyl-1,3,2-dioxaborolan-2-yl)-7,8-dihydro-4H-pyrazolo[1,5-a][1,4]diazepine-2,5(6H)-dicarboxylate (970 mg, 1.0 equiv) in THF (10 mL) and H₂O (3 mL) were added H₂O₂ (1.10 g, 30% purity, 4.3 equiv) and NaOH (178 mg, 2.0 equiv). The reaction was stirred at 20° C. for 1 hour. The reaction was quenched with saturated Na2SO3 solution (10 mL) and extracted with DCM (10×3 mL). The combined organic layers were washed with brine (30 mL), dried over anhydrous sodium sulfate, and concentrated to afford the title compound (550 mg, 26% yield) as yellow solid; LCMS (ESI, M+1): m/z=326.1.

Step E 5-tert-butyl 2-ethyl 3-methoxy-7,8-dihydro-4H-pyrazolo[1,5-a][1,4]diazepine-2,5(6H)-dicarboxylate: To a solution of 5-tert-butyl 2-ethyl 3-hydroxy-7,8-dihydro-4H-pyrazolo[1,5-a][1,4]diazepine-2,5(6H)-dicarboxylate (540 mg, 1.0 equiv) in DMF (5 mL) were added K2CO₃ (688 mg, 3.0 equiv) and Mel (2.36 g, 10 equiv). The reaction was stirred at 20° C. for 4 hours. The reaction was quenched by addition of NH₄Cl aqueous solution (4 mL) at 20° C. and extracted with EtOAc (3×3 mL). The combined organic layers were dried over anhydrous sodium sulfate, concentrated, and purified by reversed phase HPLC (0.1% FA condition) to afford the title compound (260 mg, 40% yield) as yellow solid.

Step F. 5-(tert-butoxycarbonyl)-3-methoxy-5,6,7,8-tetrahydro-4H-pyrazolo[1,5-a][1,4]diazepine-2-carboxylic acid: To a solution of 5-tert-butyl 2-ethyl 3-methoxy-7,8-dihydro-4H-pyrazolo[1,5-a][1,4]diazepine-2,5(6H)-dicarboxylate (100 mg, 1.0 equiv) in THE (1 mL) and H₂O (0.3 mL) was added NaOH (118 mg, 10 equiv). The reaction was stirred at 20° C. for 12 hours. The pH of the residue was adjusted to 4 with HCl (2 M). The mixture was extracted with EtOAc (3×3 mL). The combined organic layers were washed with brine (2×5 mL), dried over anhydrous sodium sulfate, and concentrated to afford the title compound (70.0 mg, 65% yield) as yellow solid; LCMS (ESI, M-73): m/z=237.7.

Step G. tert-butyl 3-methoxy-2-(methylcarbamoyl)-7,8-dihydro-4H-pyrazolo[1,5-a][1,4]diazepine-5(6H)-carboxylate: To a solution of 5-tert-butoxycarbonyl-3-methoxy-4,6,7,8-tetrahydropyrazolo[1,5-a][1,4]diazepine-2-carboxylic acid (60.0 mg, 1.0 equiv) and methanamine (65.0 mg, 5.0 equiv) in DCM (1 mL) were added HATU (110 mg, 1.5 equiv) and N,N-diethylpropan-2-amine (199 mg, 8.0 equiv). The reaction was stirred at 20° C. for 1 hour. The mixture was filtered and purified by reversed phase HPLC (0.1% FA condition) to afford the title compound (60.0 mg, 96% yield) as white solid; LCMS (ESI, M+1): m/z=325.3.

Step H. 3-methoxy-N-methyl-5,6,7,8-tetrahydro-4H-pyrazolo[1,5-a][1,4]diazepine-2-carboxamide: The mixture of tert-butyl 3-methoxy-2-(methylcarbamoyl)-7,8-dihydro-4H-pyrazolo[1,5-a][1,4]diazepine-5(6H)-carboxylate (50.0 mg, 1.0 equiv) in HCl•dioxane (4 M, 1 mL, 26 equiv) was stirred at 20° C. for 0.5 hours. The mixture was concentrated to afford the title compound (50 mg, crude, HCl) as white solid.

The last two steps were performed according to Example 248. The title compound was obtained as off-white solid. 1H NMR (400 MHz, METHANOL-d4) δ=7.57-7.48 (m, 1H), 7.15 (t, J=9.6 Hz, 1H), 6.97 (s, 2H), 5.66-5.45 (m, 1H), 5.04-4.93 (m, 1H), 4.71 (br t, J=14.8 Hz, 1H), 4.54-4.36 (m, 4H), 4.32-4.21 (m, 1H), 4.11-3.96 (m, 2H), 3.91-3.77 (m, 6H), 3.70 (d, J=17.6 Hz, 1H), 3.59-3.47 (m, 1H), 3.40-3.34 (m, 3H), 3.28-3.12 (m, 2H), 2.88 (s, 3H), 2.80-2.50 (m, 3H), 2.48-2.37 (m, 1H), 2.36-2.12 (m, 4H), 2.07-1.95 (m, 1H), 1.10 (t, J=7.2 Hz, 3H); LCMS (ESI, M+1): m/z=703.3.

### Example 321

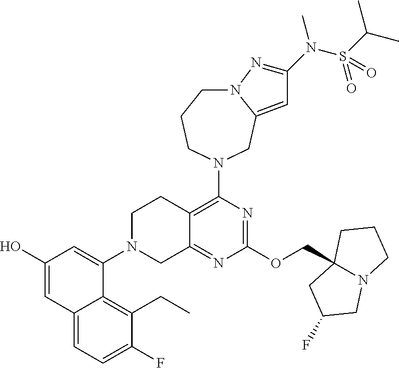 [SMILES: CCc1c(F)ccc2cc(O)cc(N3CCc4c(nc(OC[C@@]56CCCN5C[C@H](F)C6)nc4N4CCCn5nc(N(C)S(=O)(=O)C(C)C)cc5C4)C3)c12]

### N-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-methylpropane-2-sulfonamide

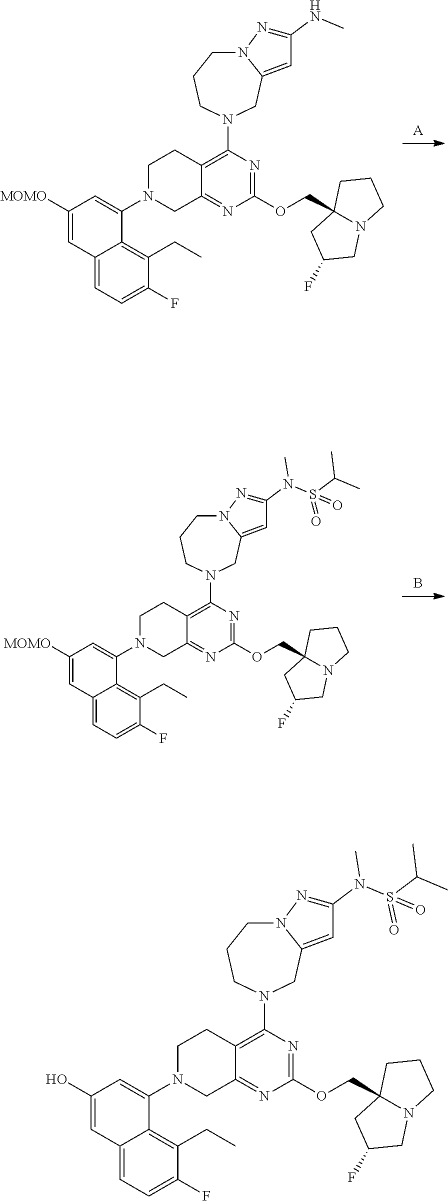

Step A. N-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-methylpropane-2-sulfonamide: To a mixture of 5-(7-(8-ethyl-7-fluoro-3-(methoxymethoxy)naphthalen-1-yl)-2-(((2R,7aS)-2-fluorotetrahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepin-2-amine (100 mg, 1.0 equiv) in pyridine (138 mg, 12 equiv) was added propane-2-sulfonyl chloride (62.1 mg, 3.0 equiv) in THF (141 μL). The reaction was stirred at 20° C. for 4 hours. The mixture was concentrated and purified by reversed phase flash chromatography [C18, 0.1% formic acid condition] to afford the title compound (64.0 mg, 55% yield) as yellow solid; LCMS (ESI, M+1): m/z=795.6.

Step B. N-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-methylpropane-2-sulfonamide: To a mixture of N-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-methylpropane-2-sulfonamide (59.0 mg, 1.0 equiv) in ACN (0.50 mL) was added HCl-MeOH (4M, 1.0 mL, 54 equiv). The reaction was stirred at 20° C. for 0.5 hours. The mixture was concentrated, dissolved in methanol (3.0 mL), neutralized with solid NaHCO₃, filtered, and purified by prep-HPLC [Phenomenex Luna C18 150 x 25 mm×10 μm; A: water (FA), B: ACN; B %: 20%-50% over 10 min] and lyophilized to afford the title compound (18.0 mg, 31% yield) as orange solid (FA salt). 1H NMR (400 MHz, dimethylsulfoxide-d6) δ=9.83-9.60 (m, 1H), 7.59 (dd, J=6.0, 8.8 Hz, 1H), 7.24 (t, J=9.6 Hz, 1H), 6.99 (s, 2H), 6.15 (s, 1H), 5.38-5.09 (m, 1H), 4.91 (br d, J=16.4 Hz, 1H), 4.69 (br d, J=16.0 Hz, 1H), 4.47-4.26 (m, 2H), 4.16-4.01 (m, 1H), 3.97-3.75 (m, 4H), 3.59 (br d, J=17.6 Hz, 1H), 3.54-3.46 (m, 1H), 3.41 (br d, J=7.6 Hz, 2H), 3.22 (s, 3H), 3.18-3.03 (m, 4H), 3.02-2.95 (m, 1H), 2.86-2.75 (m, 1H), 2.64 (br d, J=13.6 Hz, 1H), 2.16 (br s, 1H), 2.11-1.84 (m, 5H), 1.83-1,65 (m, 3H), 1.20-1.14 (m, 6H), 1.07 (br t, J=7.2 Hz, 3H); 19F NMR (376 MHz, dimethylsulfoxide-d6) δ=−121.393, -172.014; LCMS (ESI, M+1): m/z=751.5.

### Example 322

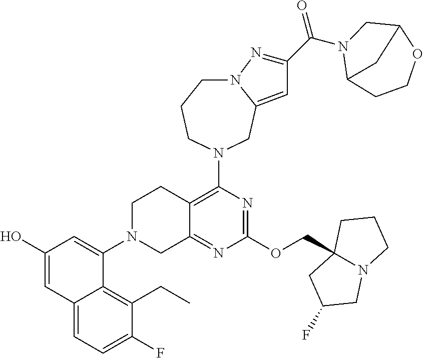 [SMILES: CCc1c(F)ccc2cc(O)cc(N3CCc4c(nc(OC[C@@]56CCCN5C[C@H](F)C6)nc4N4CCCn5nc(C(=O)N6CC7CC6CCO7)cc5C4)C3)c12]

### 2-oxa-6-azabicyclo[3.2.1]octan-6-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

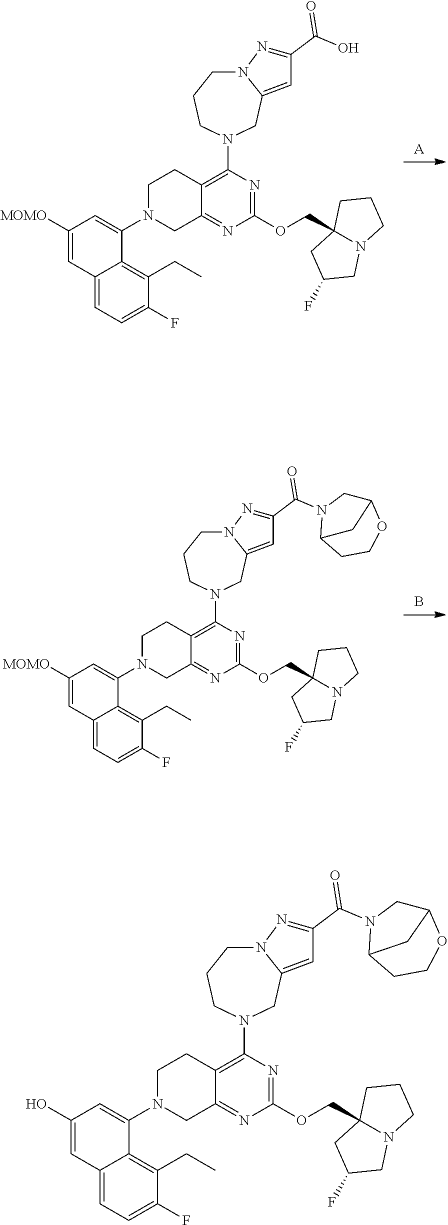

Synthesized according to Example 233 except that HCl instead of TFA was used in the last step. The title compound was obtained as white solid (31.7 mg, 21% yield); 1H NMR (400 MHz, METHANOL-d4) δ=7.51 (dd, J=6.0, 8.8 Hz, 1H), 7.20-7.09 (m, 1H), 7.01-6.88 (m, 2H), 6.78-6.68 (m, 1H), 5.34-5.01 (m, 2H), 5.00-4.87 (m, 1H), 4.64-4.44 (m, 4H), 4.33-3.90 (m, 6H), 3.86-3.62 (m, 4H), 3.58-3.50 (m, 1H), 3.44-3.38 (m, 1H), 3.24-3.11 (m, 6H), 3.02-2.89 (m, 1H), 2.79-2.65 (m, 1H), 2.42-2.03 (m, 5H), 1.99 (br s, 7H), 1.20-0.99 (m, 3H); LCMS (ESI, M+1): m/z =755.7.

### Example 323

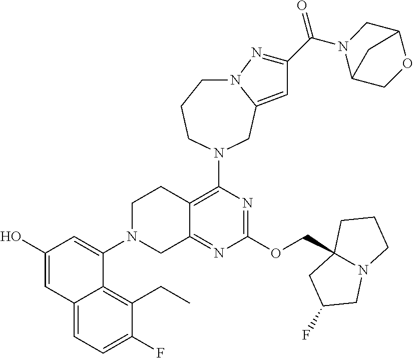 [SMILES: CCc1c(F)ccc2cc(O)cc(N3CCc4c(nc(OC[C@@]56CCCN5C[C@H](F)C6)nc4N4CCCn5nc(C(=O)N6CC7CC6CO7)cc5C4)C3)c12]

### (2-oxa-5-azabicyclo[2.2.1]heptan-5-yl)(5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

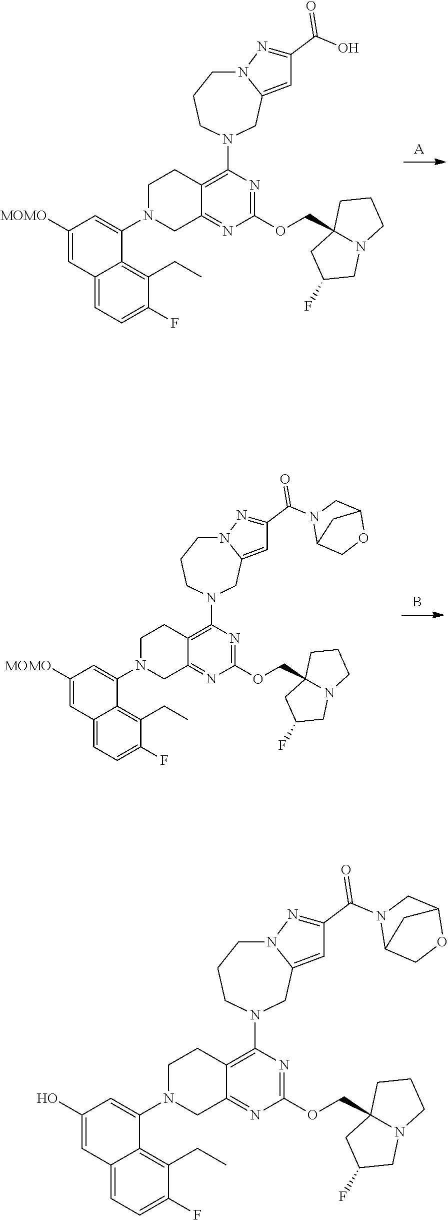

Synthesized according to Example 233. The title compound was obtained as yellow solid. 15 (t, J=12.0 Hz, 1H), 7.03-6.93 (m, 2H), 6.73 (d, J=8.0 Hz, 1H), 5.35-5.20 (m, 1H), 5.10-4.95 (m, 1H), 4.69 (br d, J=4.0 Hz, 1H), 4.60-4.50 (m, 2H), 4.33-4.17 (m, 1H), 4.16-3.97 (m, 5H), 3.95-3.86 (m, 3H), 3.73-3.64 (m, 1H), 3.61-3.52 (m, 2H), 3.45-3.38 (m, 2H), 3.27-3.11 (m, 5H), 3.03-2.94 (m, 1H), 2.74 (br d, J=16.0 Hz, 1H), 2.44-2.02 (m, 6H), 2.01-1.83 (m, 5H), 1.19-1.06 (m, 3H); LCMS (ESI, M+1). m/z=741.6.

### Example 324

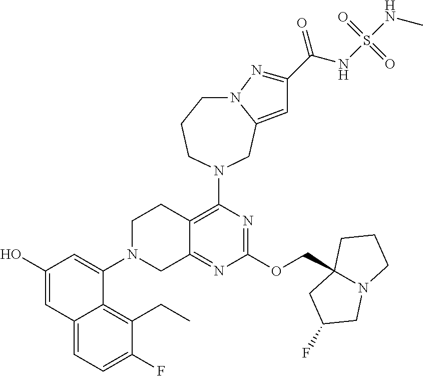 [SMILES: CCc1c(F)ccc2cc(O)cc(N3CCc4c(nc(OC[C@@]56CCCN5C[C@H](F)C6)nc4N4CCCn5nc(C(=O)NS(=O)(=O)NC)cc5C4)C3)c12]

### 7-(8-ethyl-7-fluoro-3-hydroxy-1-naphthyl)-2-(((2R,7aS)-2-fluorotetrahydro-1H-pyrrolizin-7a(5H)-yl)methoxy)-4-(2-((methylsulfamoyl)amino)methyl-7,8-dihydro-4H-pyrazolo[1,5-a][1,4]diazepin-5(6H)-yl)-6,8-dihydro-5H-pyrido[3,4-d]pyrimidine

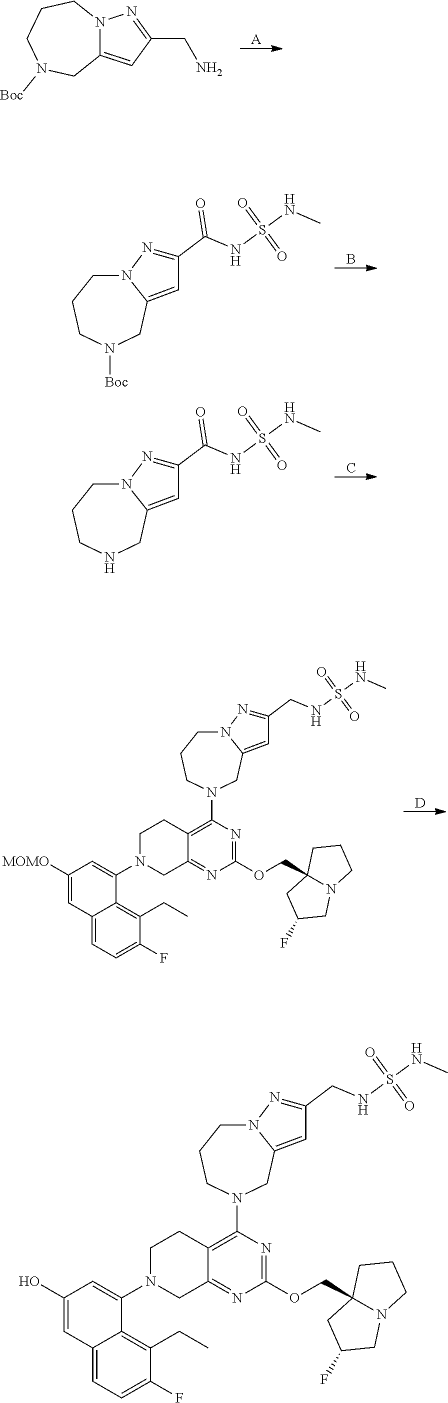

Step A. tert-butyl 2-(((N-methylsulfamoyl)amino)methyl)-7,8-dihydro-4H-pyrazolo[1,5-a][1,4]diazepine-5(6H)-carboxylate: To a solution of tert-butyl 2-(aminomethyl)-7,8-dihydro-4H-pyrazolo[1,5-a][1,4]diazepine-5(6H)-carboxylate (140 mg, 1.0 equiv) and methylsulfamoyl chloride (102 mg, 1.5 equiv) in DCM (2.0 mL) was added TEA (159 mg, 3.0 equiv). The reaction was stirred at 20° C. for 1 hour. The mixture was diluted with water (15 mL) and extracted with ethyl acetate (3×6 mL). The combined organic layers were dried over anhydrous sodium sulfate, concentrated, and purified by reversed phase HPLC (0.1% FA condition) to afford the title compound (115 mg, 60% yield) as white oil; LCMS (ESI, M+1): m/z =360.0.

Step B. 2-[(methylsulfamoylamino)methyl]-5,6,7,8-tetrahydro-4H-pyrazolo[1,5-a][1,4]diazepine: To a solution of tert-butyl 2-(((N-methylsulfamoyl)amino)methyl)-7,8-dihydro-4H-pyrazolo[1,5-a][1,4]diazepine-5(6H)-carboxylate (95.0 mg, 1.0 equiv) in MeCN (1.0 mL) was added HCl/dioxane (4 M, 1.0 mL, 15 equiv). The reaction was stirred at 25° C. for 0.5 hours. The mixture was concentrated under vacuum to afford the title compound (110 mg, crude) as white solid and used into next step without further purification.

The last two steps were performed according to Example 248. The title compound was obtained as off-white solid. 1H NMR (400 MHz, METHANOL-d4) δ=7.51 (dd, J=5.6, 8.8 Hz, 1H), 7.19-7.10 (m, 1H), 6.96 (br d, J=6.4 Hz, 2H), 6.29 (s, 1H), 5.37-5.19 (m, 1H), 4.85 (br d, J=7.2 Hz, 2H), 4.47-4.37 (m, 2H), 4.16-3.99 (m, 7H), 3.73-3.64 (m, 1H), 3.55-3.47 (m, 1H), 3.45-3.34 (m, 2H), 3.25-3.12 (m, 5H), 3.05-2.96 (m, 1H), 2.72 (br d, J=11.6 Hz, 1H), 2.55 (s, 3H), 2.32-2.13 (m, 3H), 2.12-1.93 (m, 4H), 1.91-1.83 (m, 1H), 1.13 (br t, J=6.8 Hz, 3H); LCMS (ESI, M+1): m/z=738.4.

### Example 325

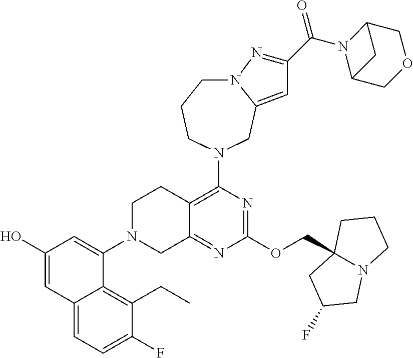 [SMILES: CCc1c(F)ccc2cc(O)cc(N3CCc4c(nc(OC[C@@]56CCCN5C[C@H](F)C6)nc4N4CCCn5nc(C(=O)N6C7COCC6C7)cc5C4)C3)c12]

### 3-oxa-6-azabicyclo[3.1.1]heptan-6-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

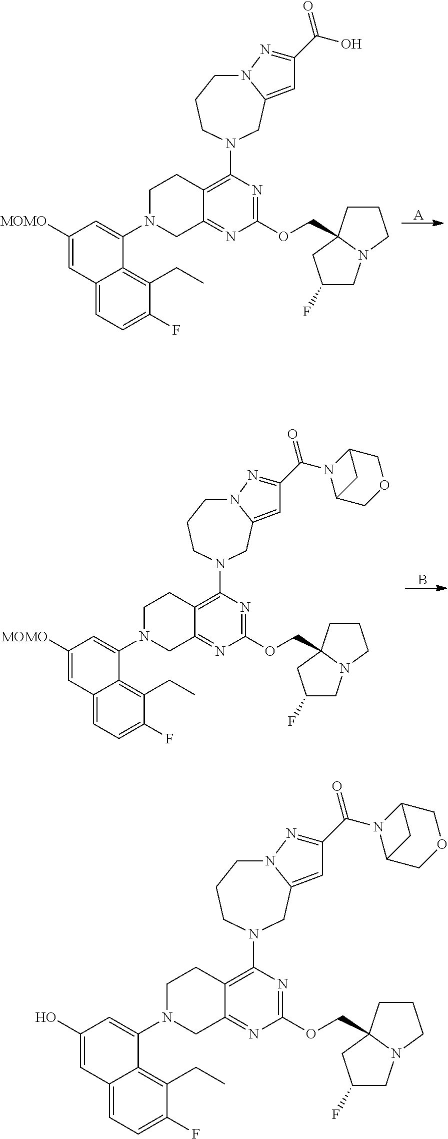

Synthesized according to Example 233 except that HCl instead of TFA was used in the last step. The title compound was obtained as off-white solid. 1H NMR (400 MHz, METHANOL-d4) δ=7.51 (dd, J=5.6, 8.8 Hz, 1H), 7.14 (t, J=9.2 Hz, 1H), 7.01-6.95 (m, 2H), 6.72 (s, 1H), 5.36-5.14 (m, 1H), 5.09 (br s, 1H), 5.05-4.92 (m, 1H), 4.51 (br s, 3H), 4.28-4.14 (m, 3H), 4.11-3.97 (m, 4H), 3.91 (br dd, J=10.0, 16.4 Hz, 2H), 3.71-3.61 (m, 1H), 3.57-3.50 (m, 1H), 3.49-3.35 (m, 2H), 3.24-3.12 (m, 5H), 3.01-2.93 (m, 1H), 2.80-2.69 (m, 2H), 2.38-2.04 (m, 5H), 2.03-1.70 (m, 5H), 1.12 (br s, 3H); LCMS (ESI, M+1): m/z=741.4.

### Example 326

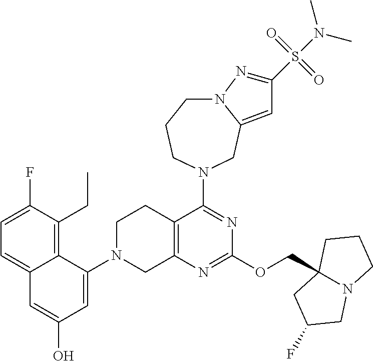 [SMILES: CCc1c(F)ccc2cc(O)cc(N3CCc4c(nc(OC[C@@]56CCCN5C[C@H](F)C6)nc4N4CCCn5nc(S(=O)(=O)N(C)C)cc5C4)C3)c12]

### 5-(7-(8-ethyl-7-fluoro-3-hydroxynaphthalen-1-yl)-2-(((2R,7aS)-2-fluorohexahydro-11H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-sulfonamide

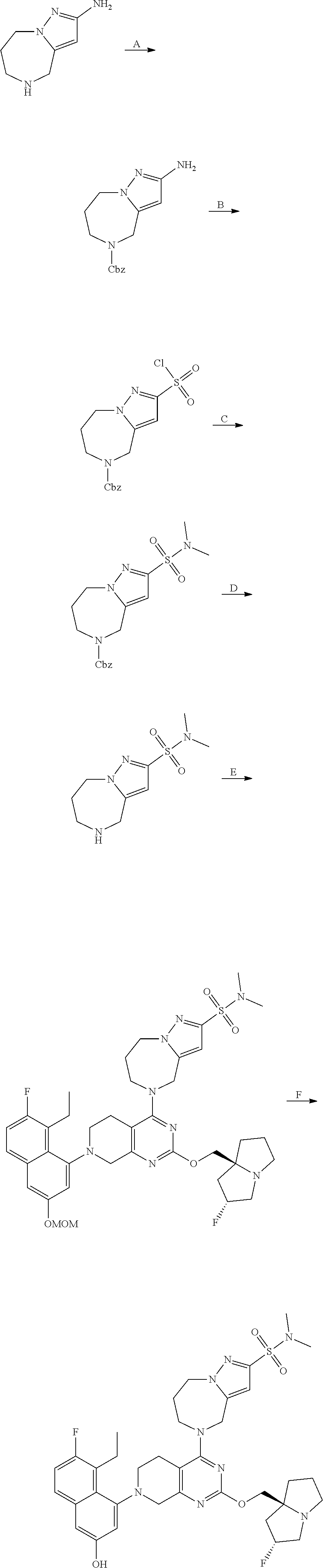

Step A. benzyl 2-amino-7,8-dihydro-4H-pyrazolo[1,5-a][1,4]diazepine-5(6H)-carboxylate: To a mixture of 5,6,7,8-tetrahydro-4H-pyrazolo[1,5-a][1,4]diazepin-2-amine (500 mg, 1.0 equiv, HCQ), TEA (1.07 g, 4.0 equiv) in THE (10 mL) was added CbzCl (452 mg, 1.0 equiv) at 0° C. The reaction was stirred at 20° C. for 2 hours. To the mixture was added NaHCO₃ aqueous solution (20 mL) and the mixture was extracted with EtOAc (3×10 mL). The combined organic layers were washed with brine, dried over anhydrous sodium sulfate, concentrated, and purified by reversed phase flash chromatography [C18, 0.1% formic acid condition] to afford the title compound (450 mg, 56% yield) as yellow oil; 1H NMR (400 MHz, chloroform-d) δ=7.39-7.29 (m, 5H), 5.67-5.45 (m, 1H), 5.13-5.09 (m, 2H), 4.46-4.39 (m, 2H), 4.23-4.18 (m, 2H), 3.77-3.71 (m, 2H), 1.97-1.85 (m, 2H).

Step B. benzyl 2-(chlorosulfonyl)-7,8-dihydro-4H-pyrazolo[1,5-a][1,4]diazepine-5(6H)-carboxylate: To a mixture of benzyl 2-amino-7,8-dihydro-4H-pyrazolo[1,5-a][1,4]diazepine-5(6H)-carboxylate (500 mg, 1.0 equiv) in ACN (40 mL) at 0° C. was added a solution of HCl (2.55 g, 37% purity, 14.8 equiv) in H₂O (0.9 mL) followed by an aqueous solution of NaNO2 (241 mg, 2.0 equiv) in H₂O (0.8 mL). After stirring at 0° C. for 45 minutes, to the mixture were added AcOH (945 mg, 9.0 equiv), CuCl (86.4 mg, 0.5 equiv) and CuCl2 (141 mg, 0.6 equiv) and the reaction was purged with S02 gas at 0° C. for 25 minutes. The reaction was stirred at 0-15° C. for 12 hours. The mixture was poured into ice-water (10 mL) and extracted with EtOAc (3×15 mL). The combined organic layers were washed with brine, dried over anhydrous sodium sulfate, and concentrated to afford the title compound (600 mg, crude) as dark green oil.

Step C. benzyl 2-(N,N-dimethylsulfamoyl)-7,8-dihydro-4H-pyrazolo[1,5-a][1,4]diazepine-5(6H)-carboxylate: A mixture of benzyl 2-(chlorosulfonyl)-7,8-dihydro-4H-pyrazolo[1,5-a][1,4]diazepine-5(6H)-carboxylate (200 mg, 1.0 equiv) in N-methylmethanamine (2 M in THF, 6.67 mL, 24 equiv) was stirred at 20° C. for 2 hours. The mixture was concentrated, diluted with saturated NH₄Cl aqueous solution (5 mL), and extracted with EtOAc (3×10 mL). The combined organic layers were washed with brine (10 mL), dried over anhydrous sodium sulfate, concentrated, and purified by reversed phase flash chromatography [C18, 0.1% formic acid condition] to afford the title compound (80 mg, 38% yield) as yellow oil; 1H NMR (400 MHz, chloroform-d) δ=7.41-7.29 (m, 5H), 6.67-6.44 (m, 1H), 5.14-5.08 (m, 2H), 4.61-4.56 (m, 1H), 4.56-4.48 (m, 3H), 3.86-3.79 (m, 2H), 2.83-2.77 (m, 6H), 2.05-1.94 (m, 2H); LCMS (ESI, M+1): m/z=378.9.

Step D. N,N-dimethyl-5,6,7,8-tetrahydro-4H-pyrazolo[1,5-a][1,4]diazepine-2-sulfonamide: To a mixture of benzyl 2-(N,N-dimethylsulfamoyl)-7,8-dihydro-4H-pyrazolo[1,5-a][1,4]diazepine-5(6H)-carboxylate (80 mg, 1.0 equiv) in MeOH (5 mL) were added NH₃-MeOH (12 M, 0.5 mL, 27 equiv) and Pd/C (30 mg, 10%, 1.0 equiv) under nitrogen. The reaction was degassed and purged with hydrogen 3 times. The reaction was stirred at 20° C. for 1 hour under hydrogen (15 psi) atmosphere. The mixture was filtered and concentrated to afford the title compound (40 mg, crude) as yellow oil.

The last two steps were performed according to Example 248. The title compound was obtained as yellow oil. 1H NMR (400 MHz, dimethylsulfoxide-d6) δ=9.73-9.64 (m, 1H), 7.65-7.55 (m, 1H), 7.28-7.20 (m, 1H), 7.01-6.96 (m, 2H), 6.64-6.60 (m, 1H), 5.33-5.14 (m, 1H), 5.05-4.97 (m, 1H), 4.84-4.76 (m, 1H), 4.61-4.53 (m, 2H), 4.17-4.06 (m, 1H), 3.95-3.88 (m, 1H), 3.87-3.83 (m, 2H), 3.83-3.76 (m, 1H), 3.64-3.55 (m, 1H), 3.46-3.39 (m, 1H), 3.25-3.13 (m, 2H), 3.13-3.02 (m, 3H), 3.01-2.95 (m, 1H), 2.84-2.75 (m, 1H), 2.67-2.62 (m, 7H), 2.26-2.18 (m, 1H), 2.09-2.02 (m, 1H), 2.01-1.91 (m, 3H), 1.90-1.66 (m, 4H), 1.10-1.03 (m, 3H); 1H NMR (400 MHz, dimethylsulfoxide-d6+deuterium oxide-d2) δ=7.58-7.54 (m, 1H), 7.27-7.20 (m, 1H), 7.02-6.95 (m, 2H), 6.64-6.60 (m, 1H), 5.32-5.14 (m, 1H), 5.08-4.94 (m, 1H), 4.84-4.75 (m, 1H), 4.63-4.48 (m, 2H), 4.18-4.08 (m, 1H), 3.97-3.74 (m, 4H), 3.63-3.52 (m, 1H), 3.31-3.22 (m, 2H), 3.21-3.17 (m, 1H), 3.12-3.01 (m, 3H), 3.00 (m, 1H), 2.83-2.75 (m, 1H), 2.70-2.61 (m, 7H), 2.26-2.16 (m, 1H), 2.07-2.01 (m, 1H), 2.01-1.90 (m, 3H), 1.89-1.66 (m, 4H), 1.09-1.01 (m, 3H); 19F NMR (400 MHz, dimethylsulfoxide-d6+deuterium oside-d2) δ=−121.265, -171.924; LCMS (ESI, M+1): m/z=723.3.

### Example 327

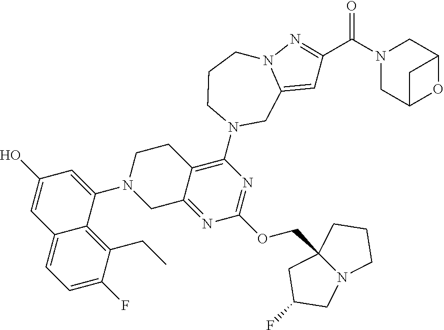 [SMILES: CCc1c(F)ccc2cc(O)cc(N3CCc4c(nc(OC[C@@]56CCCN5C[C@H](F)C6)nc4N4CCCn5nc(C(=O)N6CC7CC(C6)O7)cc5C4)C3)c12]

### 6-oxa-3-azabicyclo[3.1.1]heptan-3-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

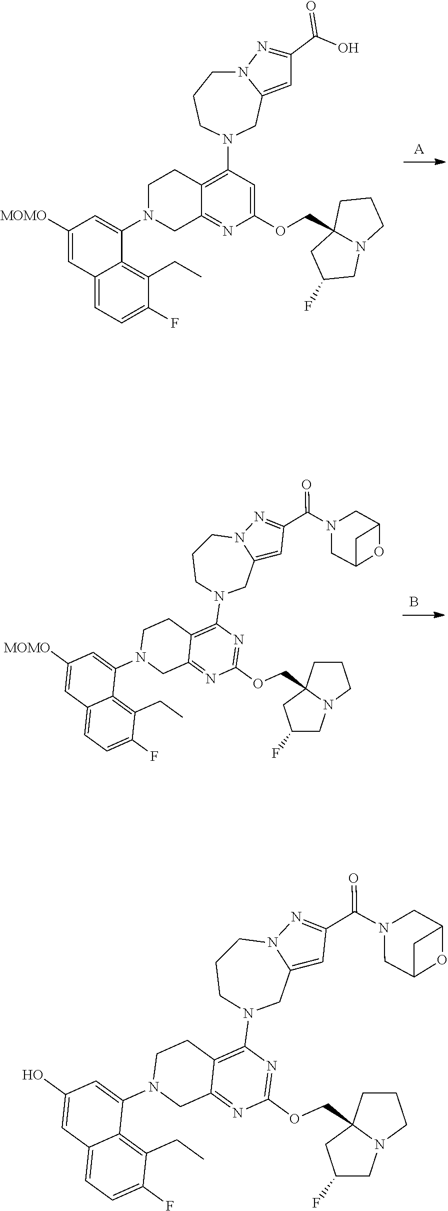

Synthesized according to Example 233. The title compound was obtained as off-white solid (TFA salt). 1H NMR (400 MHz, METHANOL-d4) δ=7.52 (dd, J=6.0, 9.2 Hz, 1H), 7.15 (t, J=9.2 Hz, 1H), 6.98 (s, 2H), 6.84-6.69 (m, 1H), 5.66-5.44 (m, 1H), 5.03-4.92 (m, 2H), 4.75-4.62 (m, 2H), 4.61-4.45 (m, 4H), 4.44-4.33 (m, 1H), 4.27-3.89 (m, 6H), 3.83 (br s, 2H), 3.77-3.66 (m, 2H), 3.62-3.52 (m, 1H), 3.49-3.35 (m, 3H), 3.29-3.12 (m, 3H), 2.79 (br d, J=14.8 Hz, 1H), 2.72-2.50 (m, 2H), 2.48-2.38 (m, 1H), 2.37-2.25 (m, 3H), 2.22-2.00 (m, 2H), 1.94-1.83 (m, 1H), 1.18-1.04 (m, 3H); LCMS (ESI, M+1): m/z=741.6.

### Example 328

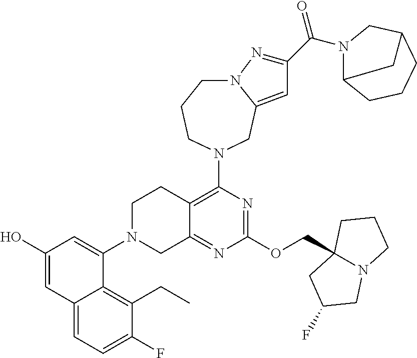 [SMILES: CCc1c(F)ccc2cc(O)cc(N3CCc4c(nc(OC[C@@]56CCCN5C[C@H](F)C6)nc4N4CCCn5nc(C(=O)N6CC7CCCC6C7)cc5C4)C3)c12]

### 6-azabicyclo[3.2.1]octan-6-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

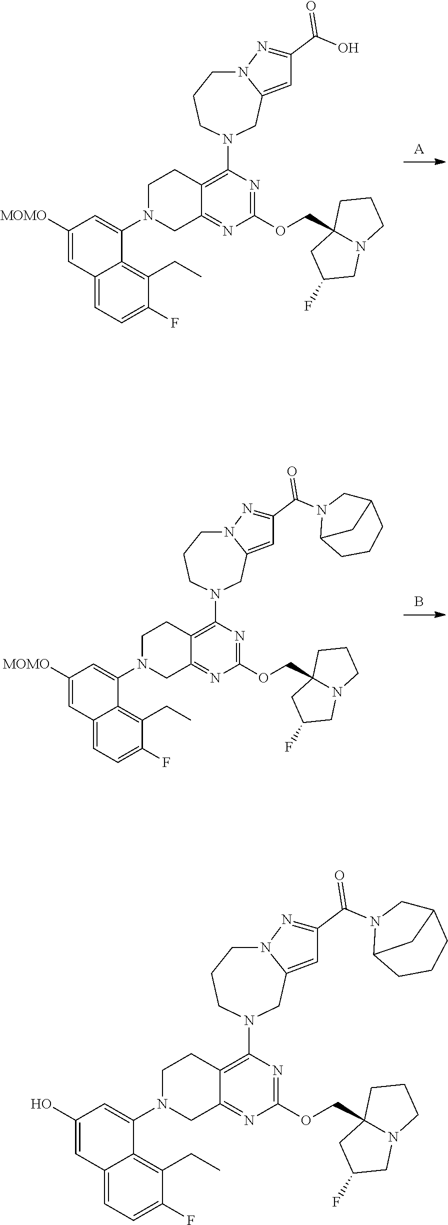

Synthesized according to Example 233 except that HCl instead of TFA was used in the last step. The title compound was obtained as off-white solid (FA salt);.1H NMR (400 MHz, METHANOL-d4) δ=7.51 (dd, J=6.0, 9.2 Hz, 1H), 7.14 (t, J=9.2 Hz, 1H), 7.01-6.88 (m, 2H), 6.78-6.60 (m, 1H), 5.46-5.23 (m, 1H), 5.09-4.89 (m, 2H), 4.62-4.45 (m, 3H), 4.31-4.10 (m, 3H), 4.10-3.85 (m, 3H), 3.72-3.50 (m, 3H), 3.46-3.34 (m, 4H), 3.27-3.05 (m, 4H), 2.74 (br d, J=14.0 Hz, 1H), 2.53-2.22 (m, 4H), 2.21-2.14 (m, 1H), 2.12-1.99 (m, 4H), 1.97-1.84 (m, 2H), 1.74-1.41 (m, 6H), 1.18-1.01 (m, 3H); LCMS (ESI, M+1): m/z=753.4.

### Example 329

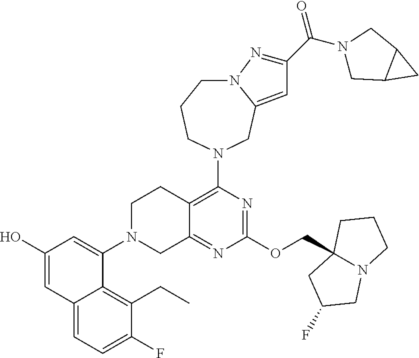 [SMILES: CCc1c(F)ccc2cc(O)cc(N3CCc4c(nc(OC[C@@]56CCCN5C[C@H](F)C6)nc4N4CCCn5nc(C(=O)N6CC7CC7C6)cc5C4)C3)c12]

### 3-azabicyclo[3.1.0]hexan-3-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

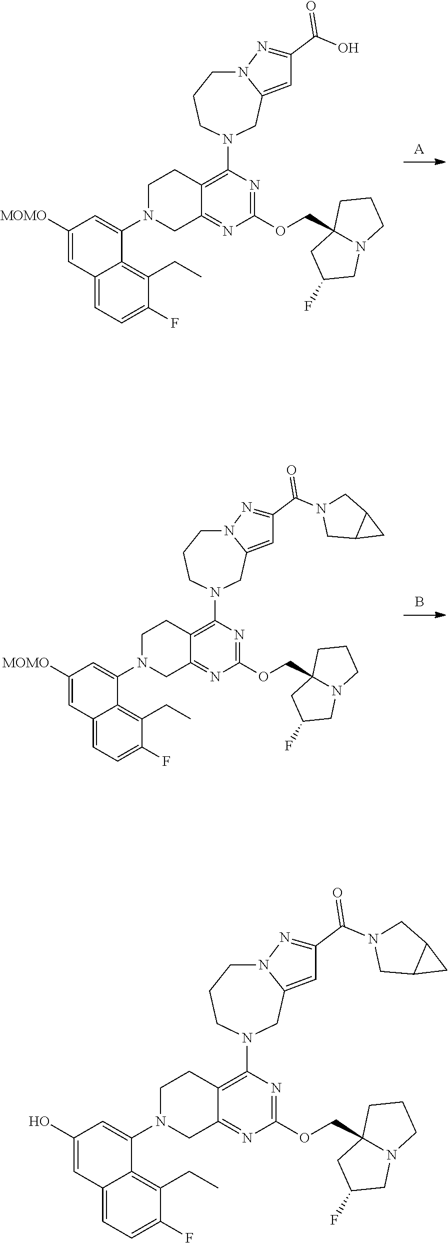

Synthesized according to Example 233 except that HCl instead of TFA was used in the last step. The title compound was obtained as white solid;.1H NMR (400 MHz, METHANOL-d4) δ=7.51 (dd, J=5.6, 9.2 Hz, 1H), 7.14 (t, J=9.2 Hz, 1H), 7.01-6.93 (m, 2H), 6.67-6.63 (m, 1H), 5.35-5.16 (m, 1H), 5.08-4.93 (m, 1H), 4.59-4.51 (m, 2H), 4.30-4.13 (m, 2H), 4.12-3.91 (m, 5H), 3.87-3.79 (m, 1H), 3.71-3.62 (m, 1H), 3.58-3.36 (m, 4H), 3.26-3.09 (m, 6H), 3.03-2.94 (m, 1H), 2.72 (br d, J=16.4 Hz, 1H), 2.38-2.11 (m, 3H), 2.11-2.02 (m, 2H), 1.99-1.80 (m, 3H), 1.70-1.57 (m, 2H), 1.15-1.05 (m, 3H), 0.80-0.72 (m, 1H), 0.10 (q, J=4.0 Hz, 1H); LCMS (ESI, M+1): m/z=725.6.

### Example 330

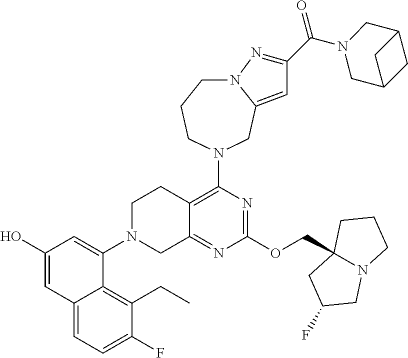 [SMILES: CCc1c(F)ccc2cc(O)cc(N3CCc4c(nc(OC[C@@]56CCCN5C[C@H](F)C6)nc4N4CCCn5nc(C(=O)N6CC7CC(C7)C6)cc5C4)C3)c12]

### 3-azabicyclo[3.1]heptan-3-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

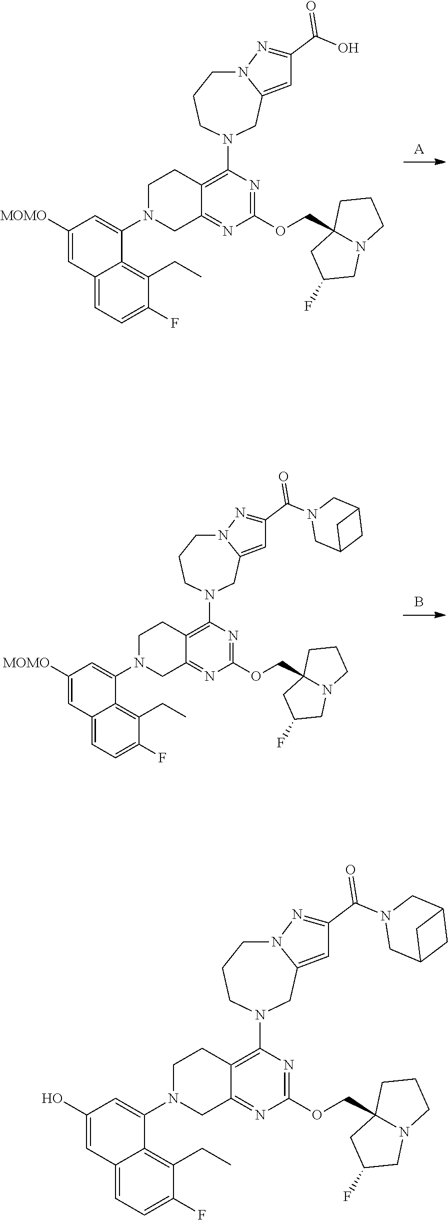

Synthesized according to Example 233 except that HCl instead of TFA was used in the last step. The title compound was obtained as off-white solid (FA salt).1H NMR (400 MHz, METHANOL-d4) δ=7.51 (dd, J=5.6, 9.2 Hz, 1H), 7.14 (t, J=9.6 Hz, 1H), 6.97 (s, 2H), 6.67 (d, J=2.4 Hz, 1H), 5.49-5.25 (m, 1H), 5.07-4.82 (m, 2H), 4.53 (br d, J=4.8 Hz, 2H), 4.30-4.16 (m, 3H), 4.15-3.98 (m, 4H), 3,89-3.75 (m, 2H), 3.68 (br d, J=17.6 Hz, 1H), 3.59-3.36 (m, 5H), 3.27-3.12 (m, 4H), 2.75 (br d, J=14.4 Hz, 1H), 2.59-2.43 (m, 2H), 2.43-1.91 (m, 10H), 1.52-1.30 (m, 2H), 1.10 (br t, J=7.2 Hz, 3H). LCMS (ESI, M+1): m/z=739.5.

### Example 331

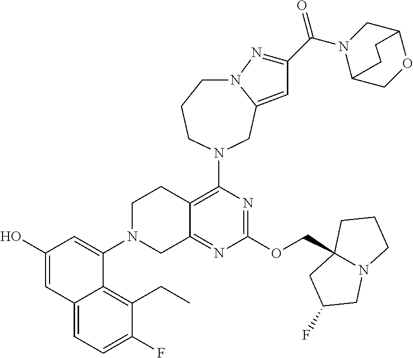 [SMILES: CCc1c(F)ccc2cc(O)cc(N3CCc4c(nc(OC[C@@]56CCCN5C[C@H](F)C6)nc4N4CCCn5nc(C(=O)N6CC7CCC6CO7)cc5C4)C3)c12]

### 2-oxa-5-azabicyclo[2.2.2]octan-5-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

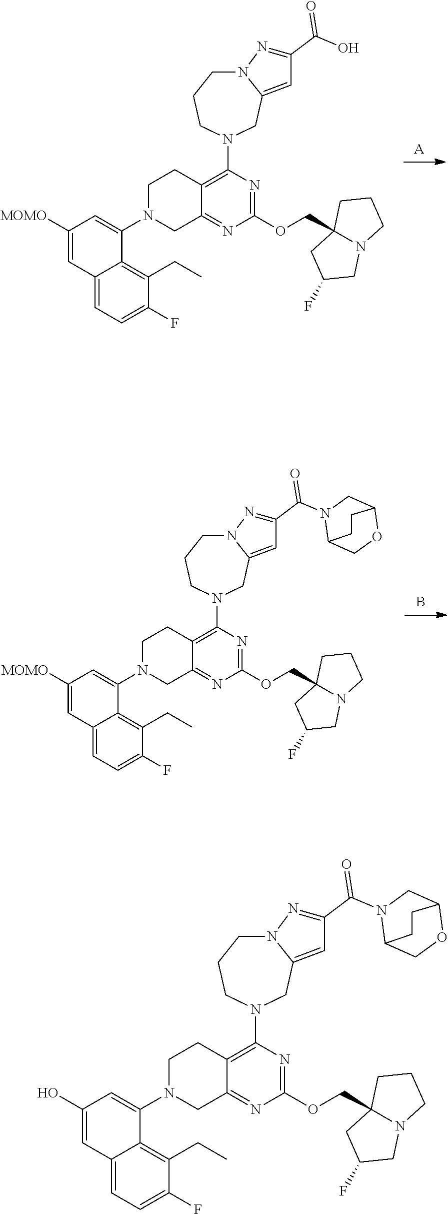

Synthesized according to Example 233 except that HCl instead of TFA was used in the last step. The title compound was obtained as off-white solid (FA salt).1H NMR (400 MHz, METHANOL-d4) δ=7.51 (dd, J=5.6, 9.2 Hz, 1H), 7.14 (t, J=9.6 Hz, 1H), 6.97 (s, 2H), 6.76-6.63 (m, 1H), 5.43-5.24 (m, 1H), 5.13-4.88 (m, 2H), 4.57-4.51 (m, 3H), 4.24-4.02 (m, 7H), 3.97 (br d, J=9.6 Hz, 1H), 3.92-3.83 (m, 1H), 3.72-3.50 (m, 3H), 3.48-3.36 (m, 3H), 3.27-3.07 (m, 5H), 2.79-2.70 (m, 1H), 2.42-2.22 (m, 3H), 2.21-2.10 (m, 3H), 2.09-1.90 (m, 5H), 1.86-1.73 (m, 1H), 1.11 (br t, J=7.2 Hz, 3H). LCMS (ESI, M+1): m/z=755.2.

### Example 332

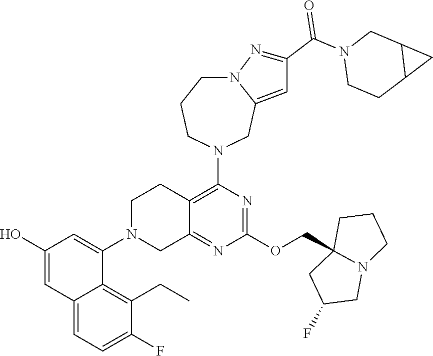 [SMILES: CCc1c(F)ccc2cc(O)cc(N3CCc4c(nc(OC[C@@]56CCCN5C[C@H](F)C6)nc4N4CCCn5nc(C(=O)N6CCC7CC7C6)cc5C4)C3)c12]

### 3-azabicyclo[4.1.0]heptan-3-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

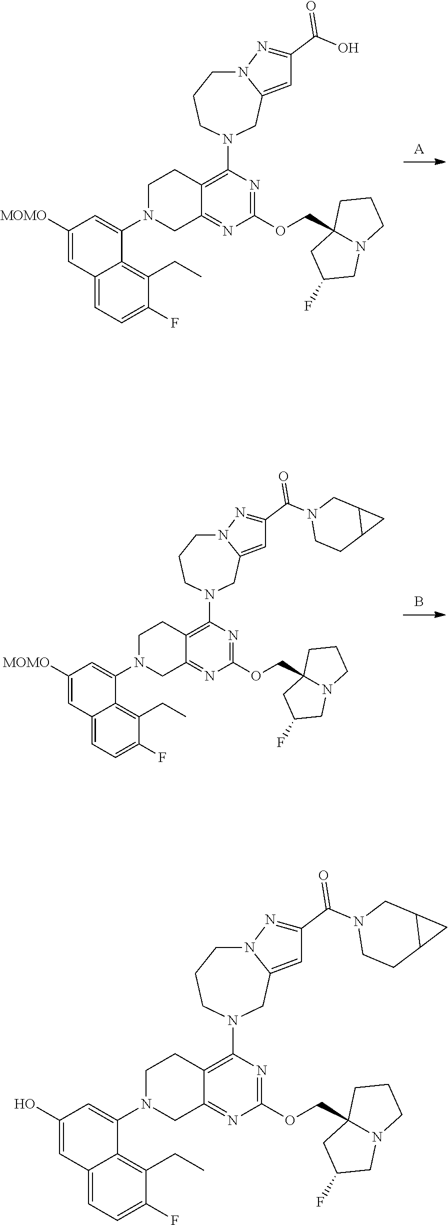

Synthesized according to Example 233 except that HCl instead of TFA was used in the last step. The title compound was obtained as off-white solid (FA salt); 1H NMR (400 MHz, METHANOL-d4) δ=7.51 (dd, J=5.6, 8.8 Hz, 1H), 7.14 (s, 1H), 6.97 (s, 2H), 6.65-6.53 (m, 1H), 5.41-5.20 (m, 1H), 5.10-4.95 (m, 1H), 4.81 (br s, 1H), 4.53 (br s, 3H), 4.32-3.97 (m, 7H), 3.89-3.61 (m, 2H), 3.58-3.35 (m, 4H), 3.27-3.03 (m, 5H), 2.79-2.66 (m, 1H), 2.50-1.65 (m, 12H), 1.10 (br t, J=6.8 Hz, 4H), 0.76-0.15 (m, 1H), LCMS (ESI, M+1): m/z=739.4.

### Example 333

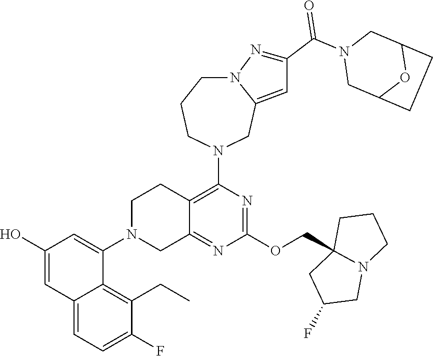 [SMILES: CCc1c(F)ccc2cc(O)cc(N3CCc4c(nc(OC[C@@]56CCCN5C[C@H](F)C6)nc4N4CCCn5nc(C(=O)N6CC7CCC(C6)O7)cc5C4)C3)c12]

### 8-oxa-3-azabicyclo[3.2.1]octan-3-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

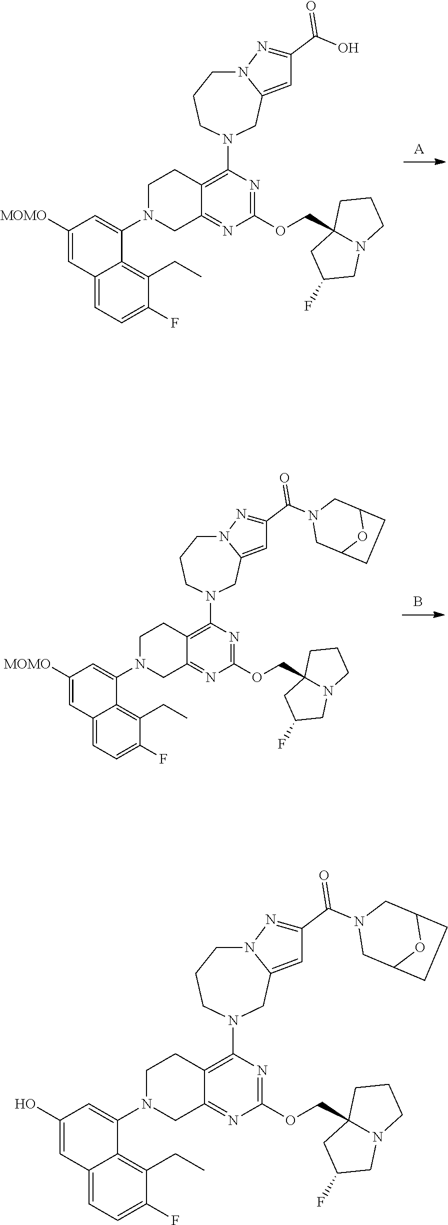

Synthesized according to Example 233 except that HCl instead of TFA was used in the last step. The title compound was obtained as off-white solid (FA salt). 1H NMR (400 MHz, METHANOL-d4) δ=7.51 (dd, J=6.0, 8.8 Hz, 1H), 7.14 (s, 1H), 6.97 (s, 2H), 6.61 (s, 1H), 5.41-5.21 (m, 1H), 5.08-4.94 (m, 1H), 4.83-4.74 (m, 1H), 4.65-4.47 (m, 3H), 4.43 (br s, 1H), 4.35-3.97 (m, 7H), 3.67 (br d, J=18.0 Hz, 1H), 3.58-3.50 (m, 1H), 3.47-3.34 (m, 4H), 3.29-3.11 (m, 4H), 3.10-3.01 (m, 2H), 2.74 (br d, J=13.6 Hz, 1H), 2.37-2.17 (m, 3H), 1.91 (br s, 9H), 1.11 (br t, J=7.2 Hz, 3H), LCMS (ESI, M+1): m/z=755.5.

### Example 334

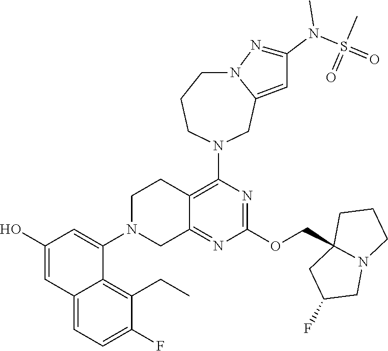 [SMILES: CCc1c(F)ccc2cc(O)cc(N3CCc4c(nc(OC[C@@]56CCCN5C[C@H](F)C6)nc4N4CCCn5nc(N(C)S(C)(=O)=O)cc5C4)C3)c12]

### N-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-methylmethanesulfonamide

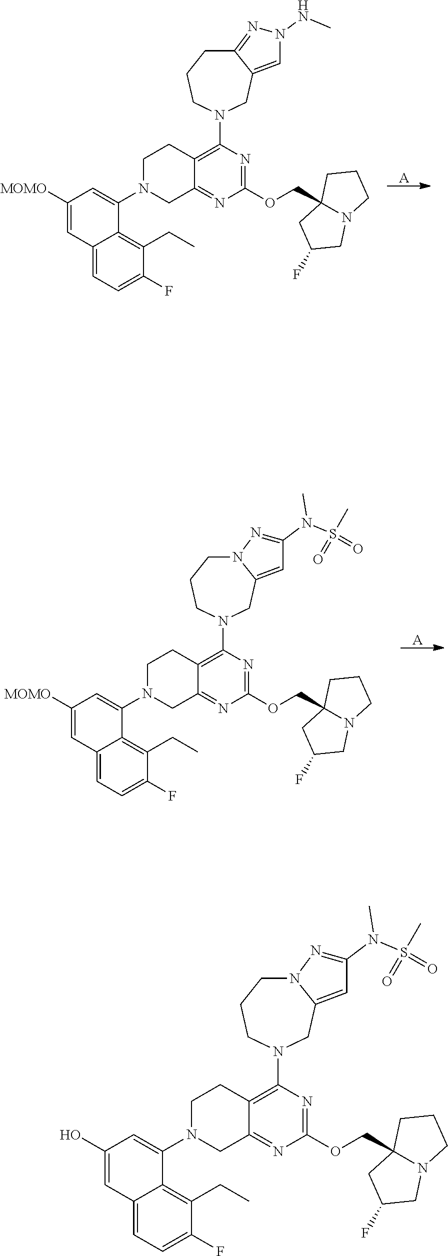

Step A. N-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-5,6,7,8-tetrahydro-4H-pyrazolo[1,5-a][1,4]diazepin-2-yl)-N-methylmethanesulfonamide: To a mixture of 5-(7-(8-ethyl-7-fluoro-3-(methoxymethoxy)naphthalen-1-yl)-2-(((2R,7aS)-2-fluorotetrahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepin-2-amine (95 mg, 1.0 equiv) in pyridine (218 mg, 20 equiv) at 0° C. was added a solution of methanesulfonic anhydride (60 mg, 2.5 equiv) in THE (0.2 mL). The reaction was stirred to 20° C. for 2 hours. To the mixture was added water (1 mL) and extracted with EtOAc (3×15 mL). The combined organic layers were washed with brine, dried over anhydrous sodium sulfate, concentrated, and purified by prep-HPLC [Phenomenex luna C18 150 x 25 mm×10 μm; A: water (FA), B: ACN; B %: 29%-59% over 10 min] to afford the title compound (50 mg, 46% yield) as yellow oil; 1H NMR (400 MHz, dimethylsulfoxide-d6) δ=7.76-7.69 (m, 1H), 7.36-7.28 (m, 2H), 7.15-7.11 (m, 1H), 6.18-6.15 (m, 1H), 5.35-5.13 (m, 3H), 4.96-4.88 (m, 1H), 4.75-4.66 (m, 1H), 4.41-4.35 (m, 2H), 4.14-4.06 (m, 1H), 3.96-3.90 (m, 1H), 3.90-3.78 (m, 3H), 3.68-3.60 (m, 1H), 3.48-3.41 (m, 4H), 3.30-3.24 (m, 2H), 3.23-3.12 (m, 5H), 3.10 (br s, 2H), 3.00-2.97 (m, 1H), 2.97-2.92 (m, 3H), 2.84-2.75 (m, 1H), 2.68-2.61 (m, 1H), 2.33 (s, 2H), 2.08-2.03 (m, 1H), 1.97-1.90 (m, 2H), 1.85-1.78 (m, 1H), 1.77 (br s, 2H), 1.20-1.15 (m, 3H); LCMS (ESI, M+1): m/z=767.5.

Step B. N-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-methylmethanesulfonamide: To a mixture of N-(5-(7-(8-ethyl-7-fluoro-3-(methoxymethoxy)naphthalen-1-yl)-2-(((2R,7aS)-2fluorohexahydro-1H-pyrrolizin-7a-yl)methoxy)-5,6,7,8-tetrahydropyrido[3,4-d]pyrimidin-4-yl)-5,6,7,8tetrahydro-4H-pyrazolo[1,5-a][1,4]diazepin-2-yl)-N-methylmethanesulfonamide (40 mg, 1.0 equiv) in ACN (0.1 mL) was added HCl•MeOH (4 M, 196 μL 15 equiv). The reaction was stirred at 15° C. for 1 hour. The mixture was concentrated, dissolved in MeOH (1 mL), neutralized with solid NaHCO₃, filtered, and purified by prep-HPLC [Phenomenex luna C18 150×25 mm×10 μm; A: water (FA), B: ACN; B %: 24%-54% over 10 min] to afford the title compound (34.2 mg) as orange solid (FA salt). 1H NMR (400 MHz, dimethylsulfoxide-d6) δ=9.79-9.60 (m, 1H), 7.63-7.54 (m, 1H), 7.28-7.20 (m, 1H), 7.02-6.93 (m, 2H), 6.20-6.14 (m, 1H), 5.35-5.15 (m, 1H), 4.95-4.87 (m, 1H), 4.74-4.66 (m, 1H), 4.45-4.32 (m, 2H), 4.14-4.04 (m, 1H), 3.99-3.88 (m, 2H), 3.86-3.80 (m, 2H), 3.63-3.56 (m, 1H), 3.25-3.23 (m, 1H), 3.18-3.14 (m, 4H), 3.13-3.05 (m, 4H), 3.04-2.99 (m, 1H), 2.96 (s, 3H), 2.87-2.77 (m, 1H), 2.68-2.60 (m, 1H), 2.21-2.00 (m, 3H), 1.99-1.78 (m, 4H), 1.77-1.67 (m, 2H), 1.10-1.02 (m, 3H); 1HNMR (400 MHz, dimethylsulfoxide-d6+deuterium oside-d2) δ=7.62-7.55 (m, 1H), 7.28-7.20 (m, 1H), 7.03-6.96 (m, 2H), 6.21-6.15 (m, 1H), 5.37-5.18 (m, 1H), 4.95-4.86 (m, 1H), 4.70 (br d, J=16 Hz, 1H), 4.41-4.32 (m, 2H), 4.16-4.05 (m, 1H), 4.03-3.80 (m, 4H), 3.46-3.38 (m, 1H), 3.25 (br d, J=6.0 Hz, 2H), 3.18-3.11 (m, 1H), 3.08 (br s, 2H), 2.97-2.92 (m, 3H), 2.90-2.81 (m, 1H), 2.70-2.61 (m, 1H), 2.20-2.01 (m, 3H), 2.02-1.80 (m, 4H), 1.80-1.69 (m, 2H), 1.09-1.00 (m, 3H); 19F NMR (400 MHz, dimethylsulfoxide-d6) δ=−121.318, -172.029; LCMS (ESI, M+1): m/z=723.3.

### Example 335

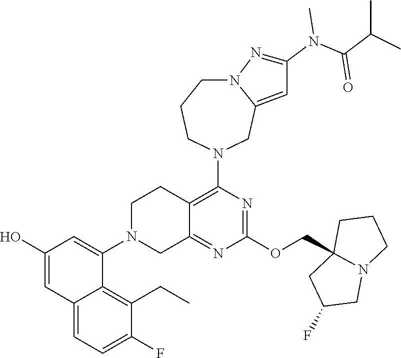 [SMILES: CCc1c(F)ccc2cc(O)cc(N3CCc4c(nc(OC[C@@]56CCCN5C[C@H](F)C6)nc4N4CCCn5nc(N(C)C(=O)C(C)C)cc5C4)C3)c12]

### N-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-methylisobutyramide

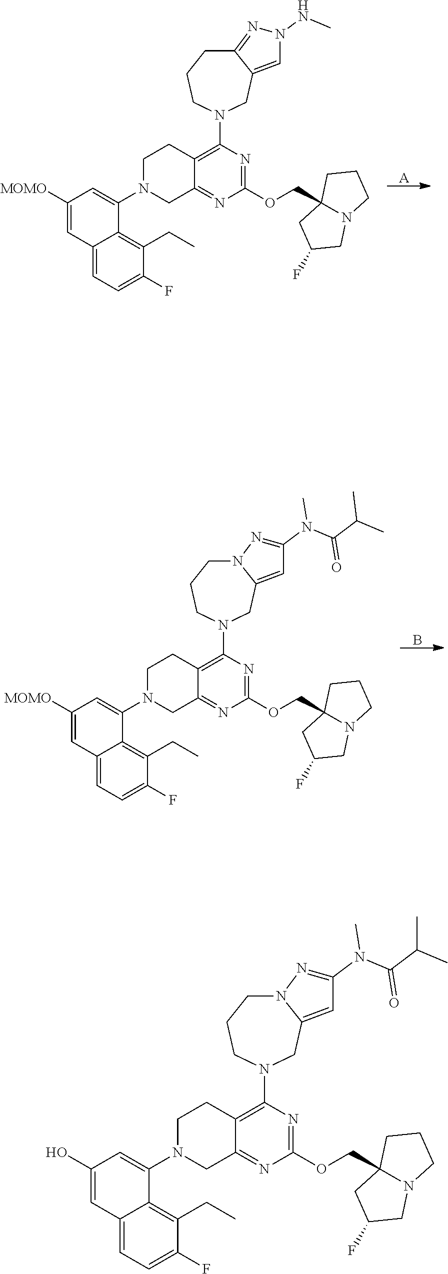

Step A. N-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-methylisobutyramide: To a mixture of 5-(7-(8-ethyl-7-fluoro-3-(methoxymethoxy)naphthalen-1-yl)-2-(((2R,7aS)-2-fluorotetrahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepin-2-amine (100 mg, 1.0 equiv) in pyridine (230 mg, 20 equiv) was added isobutyryl chloride (38.7 mg, 2.5 equiv) in THF (0.50 mL). The reaction was stirred at 20° C. for 2 hours. The mixture was concentrated and purified by reversed phase flash chromatography [C18, 0.1% formic acid condition] to afford the title compound (93.0 mg, 78% yield) as colorless oil; LCMS (ESI, M+1): m/z=759.6.

Step B. N-(5-(7-(8-ethyl-7-fluoro-3-hydroxynaphthalen-1-yl-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-methylisobutyramide: To a mixture of N-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-methylisobutyramide (88.0 mg, 1.0 equiv) in ACN (0.50 mL) was added HCl•MeOH (1.0 mL, 34 equiv). The reaction was stirred at 20° C. for 0.5 hours. The mixture was concentrated, dissolved in methanol (3.0 mL), neutralized with solid NaHCO₃, filtered, purified by prep-HPLC [Phenomenex Luna C18 150×25 mm×10 μm; A: water (FA), B: ACN; B %: 18%-48% over 15 min] and lyophilized to afford the title compound (36.7 mg) as off-white solid (FA salt). 1H NMR (400 MHz, methanol-d4) δ=7.51 (dd, J=5.6, 8.8 Hz, 1H), 7.14 (t, J=9.2 Hz, 1H), 6.96 (s, 2H), 6.21 (br s, 1H), 5.50-5.24 (m, 1H), 5.03 (br d, J=16.8 Hz, 1H), 4.48 (br s, 2H), 4.40-4.11 (m, 4H), 4.10-3.93 (m, 2H), 3.70 (br d, J=17.6 Hz, 1H), 3.56-3.35 (m, 6H), 3.26-3.08 (m, 6H), 2.81-2.69 (m, 1H), 2.67-2.56 (m, 1H), 2.46-2.24 (m, 3H), 2.21-2.03 (m, 4H), 1.99-1.84 (m, 1H), 1.21-1.06 (m, 4H), 1.00 (br s, 5H); 19F NMR (400 MHz, methanol-d4) δ=−122.940, -173.773; LCMS (ESI, M+1): m/z=715.3.

### Example 336

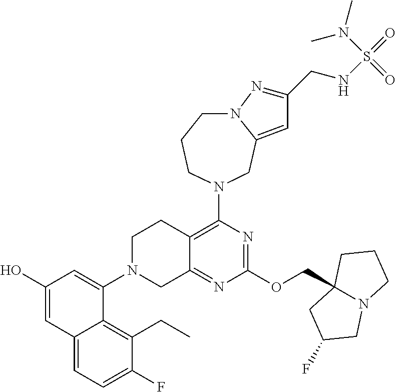 [SMILES: CCc1c(F)ccc2cc(O)cc(N3CCc4c(nc(OC[C@@]56CCCN5C[C@H](F)C6)nc4N4CCCn5nc(CNS(=O)(=O)N(C)C)cc5C4)C3)c12]

### 7-(8-ethyl-7-fluoro-3-hydroxy-1-naphthyl)-2-(((2R,7aS)-2-fluorotetrahydro-1H-pyrrolizin-7a(5H)-yl)methoxy)-4-(2-((dimethylsulfamoyl)amino)methyl-7,8-dihydro-4H-pyrazolo[1,5-a][1,4]diazepin-5(6H)-yl)-6,8-dihydro-5H-pyrido[3,4-d]pyriimidine

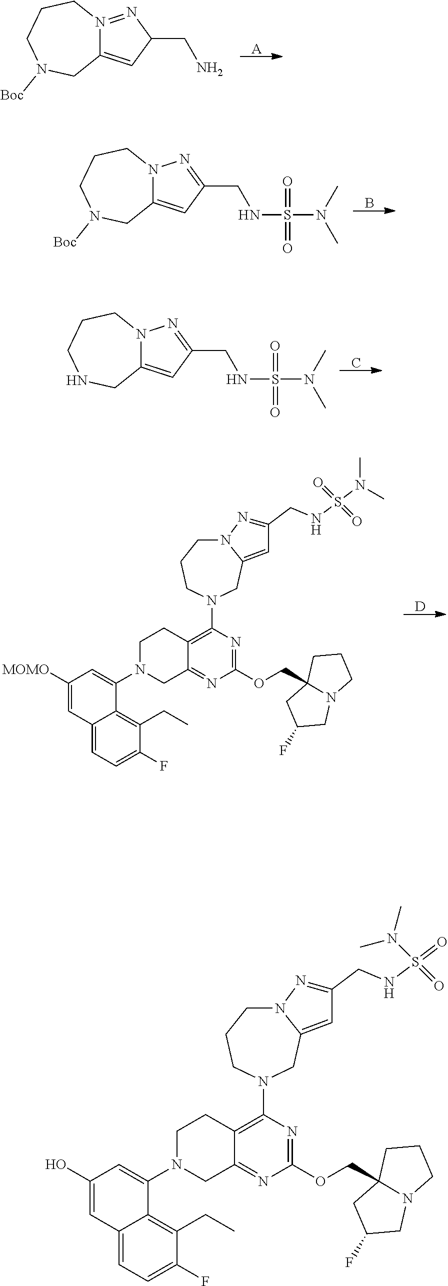

Step A. tert-butyl 2-(((N,N-dimethylsulfamoyl)amino)methyl)-7,8-dihydro-4H-pyrazolo[1,5-a][1,4]diazepine-5(6H)-carboxylate: To a solution of tert-butyl 2-(aminomethyl)-7,8-dihydro-4H-pyrazolo[1,5-a][1,4]diazepine-5(6H)-carboxylate (123 mg, 1.0 equiv) in DCM (1 mL) were added TEA (141 mg, 3.0 equiv), DABCO (156 mg, 3.0 equiv) and dimethylsulfamoyl chloride (200 mg, 3.0 equiv). The reaction was stirred at 0° C. for 12 hours. After completion, the reaction was concentrated and purified by reversed phase HPLC (0.1% FA condition) to afford the title compound (240 mg, crude) as white solid; LCMS (ESI, M+1): m/z=373.9.

Step B. 2-[(dimethylsulfamoylamino)methyl]-5,6,7,8-tetrahydro-4H-pyrazolo[1,5-a][1,4]diazepine. A solution of tert-butyl 2-(((N,N-dimethylsulfamoyl)amino)methyl)-7,8-dihydro-4H-pyrazolo[1,5-a][1,4]diazepine-5(6H)-carboxylate (100 mg, 1.0 equiv) in HCl•dioxane (4 M, 1 mL, 15 equiv) was stirred at 25° C. for 1 hour. The reaction was concentrated to afford the title compound (60.0 mg, crude, HCl) as yellow oil.

The last two steps were performed according to Example 248. The title compound was obtained as white solid (FA salt); 1H NMR (400 MHz, METHANOL-d4) δ=7.52 (dd, J=6.0, 8.8 Hz, 1H), 7.15 (t, J=9.2 Hz, 1H), 7.01-6.92 (m, 2H), 6.30 (s, 1H), 5.47-5.26 (m, 1H), 4.96-4.88 (m, 2H), 4.50-4.38 (m, 2H), 4.32-4.17 (m, 2H), 4.17-4.03 (m, 5H), 3.69 (dd, J=5.2, 17.6 Hz, 1H), 3.59-3.44 (m, 3H), 3.44-3.34 (m, 3H), 3.27-3.11 (m, 3H), 2.76 (br d, J=2.4 Hz, 1H), 2.71 (s, 6H), 2.46-2.35 (m, 1H), 2.35-2.29 (m, 1H), 2.29-2.17 (m, 2H), 2.16-2.06 (m, 2H), 2.06-1.92 (m, 2H), 1.17-1.08 (m, 3H); LCMS (ESI, M+1): m/z=752.5.

### Example 337

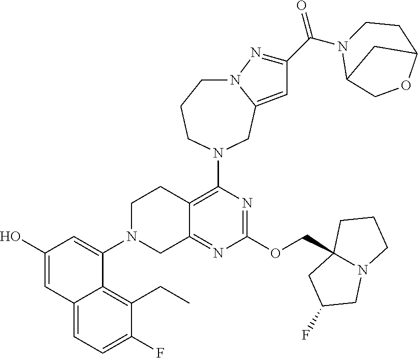 [SMILES: CCc1c(F)ccc2cc(O)cc(N3CCc4c(nc(OC[C@@]56CCCN5C[C@H](F)C6)nc4N4CCCn5nc(C(=O)N6CCC7CC6CO7)cc5C4)C3)c12]

### 6-oxa-2-azabicyclo[3.2.1]octan-2-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

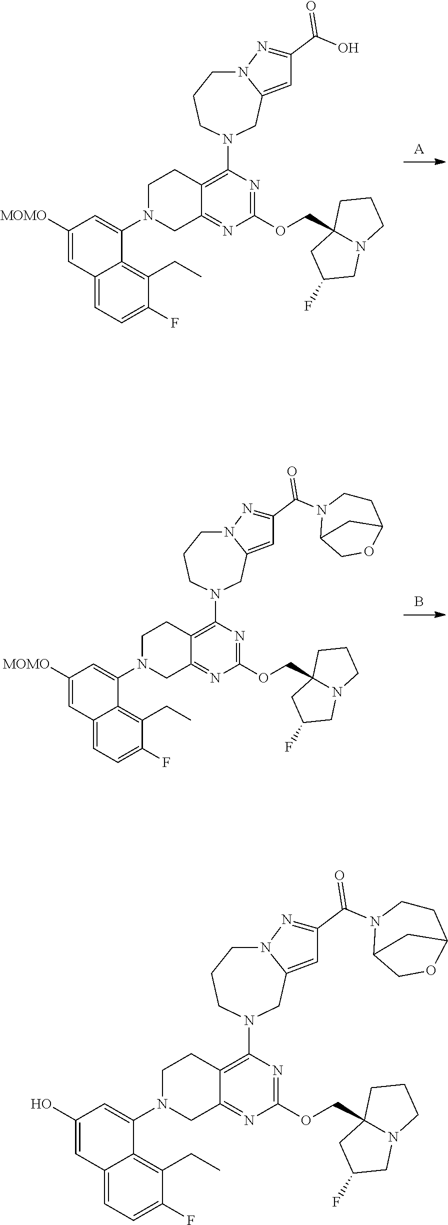

Synthesized according to Example 233 except that HCl instead of TFA was used in the last step. The title compound was obtained as off-white solid (FA salt). 1H NMR (400 MHz, METHANOL-d4) δ=7.52 (dd, J=5.6, 8.8 Hz, 1H), 7.15 (t, J=9.6 Hz, 1H), 7.00-6.94 (m, 2H), 6.66-6.59 (m, 1H), 5.54-5.43 (m, 1H), 5.36-5.24 (m, 1H), 5.05-4.88 (m, 2H), 4.61-4.15 (m, 7H), 4.14-3.85 (m, 4H), 3.73-3.37 (m, 7H), 3.30-3.10 (m, 4H), 2.74 (br d, J=15.6 Hz, 1H), 2.51-2.38 (m, 1H), 2.37-2.19 (m, 3H), 2.17-1.61 (m, 8H), 1.15-1.05 (m, 3H); LCMS (ESI, M+1): m/z=755.8.

### Example 338

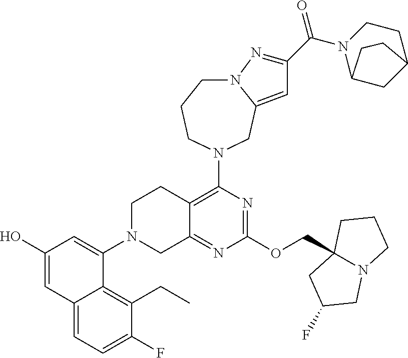 [SMILES: CCc1c(F)ccc2cc(O)cc(N3CCc4c(nc(OC[C@@]56CCCN5C[C@H](F)C6)nc4N4CCCn5nc(C(=O)N6CCC7CCC6C7)cc5C4)C3)c12]

### (1S,5S)-2-azabicyclo[3.2.1]octan-2-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

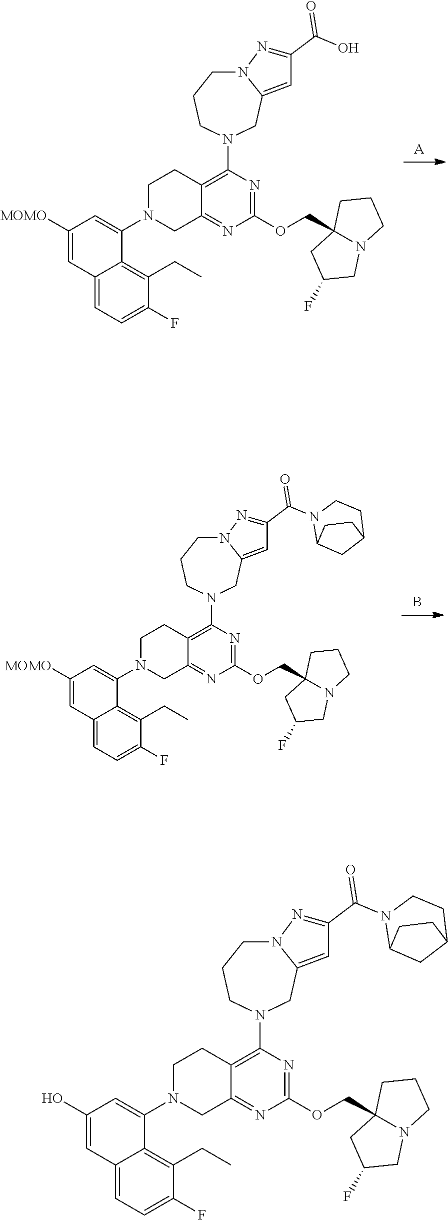

Synthesized according to Example 233 except that HCl instead of TFA was used in the last step. The title compound was obtained as white solid. 1H NMR (400 MHz, METHANOL-d4) δ=7.51 (dd, J=5.6, 8.8 Hz, 1H), 7.14 (t, J=9.2 Hz, 1H), 6.96 (s, 2H), 6.55-6.49 (m, 1H), 5.34-5.16 (m, 1H), 5.11-5.03 (m, 1H), 4.95 (br d, J=9.6 Hz, 1H), 4.62-4.49 (m, 2H), 4.36-4.13 (m, 2H), 4.12-3.90 (m, 4H), 3.66 (br d, J=17.6 Hz, 1H), 3.52 (br s, 1H), 3.46-3.33 (m, 3H), 3.27-3.12 (m, 5H), 3.11-2.93 (m, 2H), 2.72 (br d, J=14.8 Hz, 1H), 2.43 (br s, 1H), 2.32-2.22 (m, 1H), 2.20-2.01 (m, 4H), 2.00-1.88 (m, 3H), 1.88-1.77 (m, 3H), 1.72 (br d, J=11.2 Hz, 2H), 1.61 (br s, 1H), 1.53 (br s, 1H), 1.43-1.28 (m, 1H), 1.10 (q, J=7.6 Hz, 3H); LCMS (ESI, M+1): m/z=753.6.

### Example 339

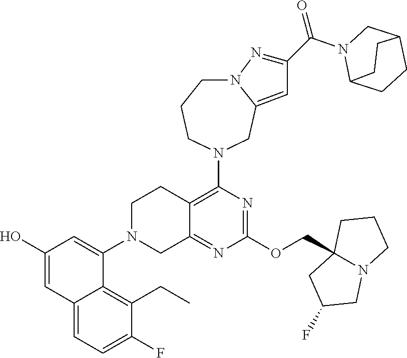 [SMILES: CCc1c(F)ccc2cc(O)cc(N3CCc4c(nc(OC[C@@]56CCCN5C[C@H](F)C6)nc4N4CCCn5nc(C(=O)N6CC7CCC6CC7)cc5C4)C3)c12]

### 2-azabicyclo[2.2.2]octan-2-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

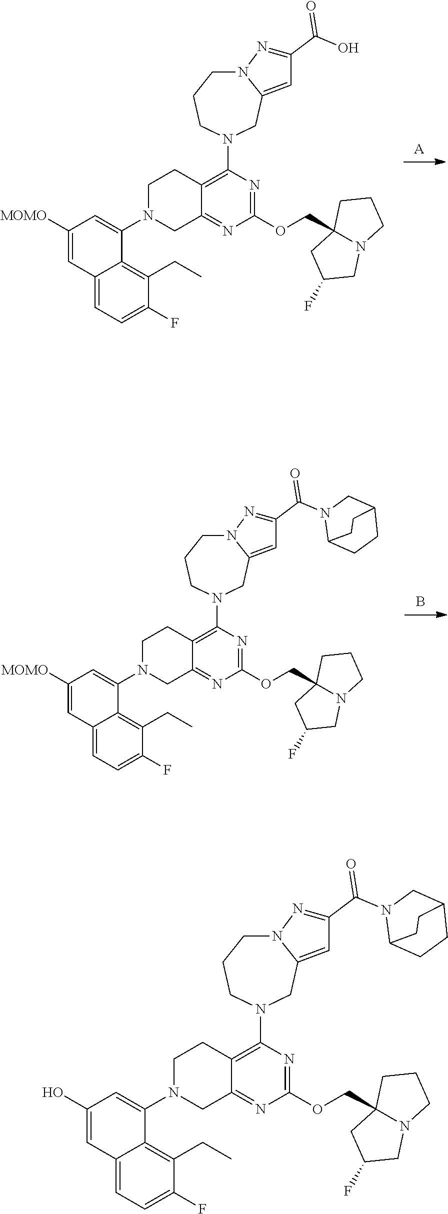

Synthesized according to Example 233 except that HCl instead of TFA was used in the last step. The title compound was obtained as yellow solid (FA salt). 1H NMR (400 MHz, METHANOL-d4) δ=7.51 (dd, J=5.6, 8.8 Hz, 1H), 7.14 (t, J=9.6 Hz, 1H), 6.97 (s, 2H), 6.69-6.55 (m, 1H), 5.45-5.27 (m, 1H), 5.04-4.93 (m, 2H), 4.69-4.46 (m, 4H), 4.27-4.13 (m, 3H), 4.09-3.98 (m, 2H), 3.86 (br d, J=11.2 Hz, 1H), 3.67 (br d, J=17.6 Hz, 1H), 3.55-3.51 (m, 2H), 3.46-3.36 (m, 4H), 3.28-3.12 (m, 3H), 2.74 (br d, J=14.4 Hz, 1H), 2.45-2.25 (m, 3H), 2.19 (br d, J=10.4 Hz, 1H), 2.14-2.00 (m, 4H), 1.99-1.87 (m, 3H), 1.82-1.66 (m, 6H), 1.15-1.04 (m, 3H); LCMS (ESI, M+1): m/z=753.8.

### Example 340

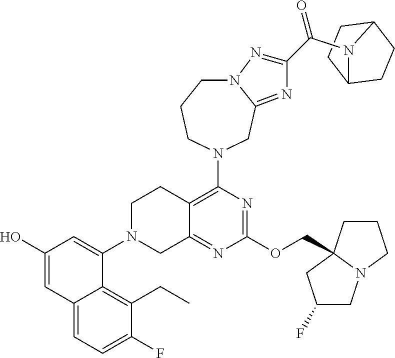 [SMILES: CCc1c(F)ccc2cc(O)cc(N3CCc4c(nc(OC[C@@]56CCCN5C[C@H](F)C6)nc4N4CCCn5nc(C(=O)N6C7CCC6CC7)nc5C4)C3)c12]

### 7-azabicyclo[2.2.1]heptan-7-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

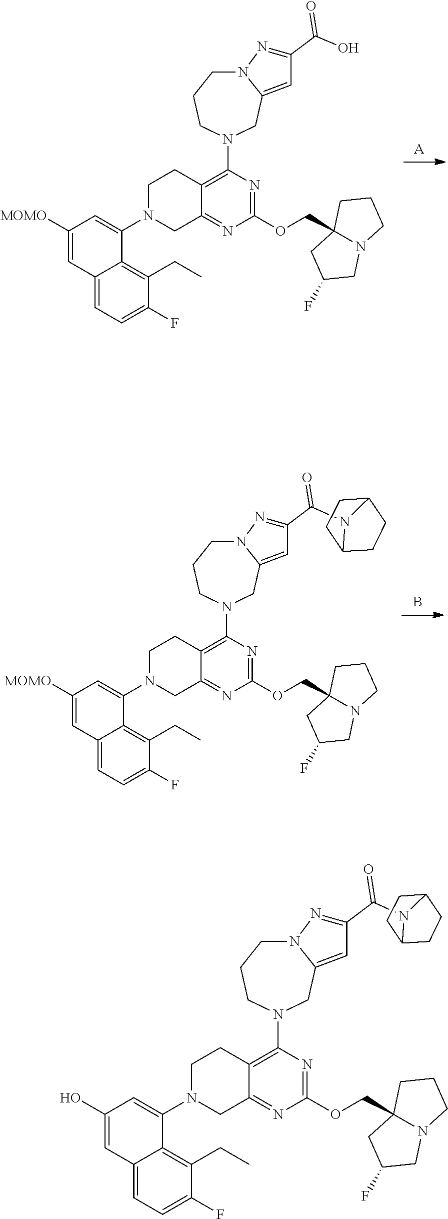

Synthesized according to Example 233 except that HCl instead of TFA was used in the last step. The title compound was obtained as white solid (FA salt). 1H NMR (400 MHz, METHANOL-d4) δ=7.51 (dd, J=5.6, 8.8 Hz, 1H), 7.14 (t, J=9.6 Hz, 1H), 6.97 (s, 2H), 6.67 (d, J=1.2 Hz, 1H), 5.43-5.18 (m, 2H), 5.06-4.94 (m, 1H), 4.85 (br s, 1H), 4.70 (br s, 2H), 4.58-4.50 (m, 2H), 4.26-4.15 (m, 2H), 4.15-4.09 (m, 1H), 4.08-3.97 (m, 2H), 3.66 (br d, J=17.6 Hz, 1H), 3.54 (br d, J=10.4 Hz, 1H), 3.44-3.34 (m, 3H), 3.28-3.13 (m, 3H), 3.12-3.05 (m, 1H), 2.73 (br d, J=14.8 Hz, 1H), 2.41-2.20 (m, 3H), 2.19-2.01 (m, 4H), 1.98-1.76 (m, 5H), 1.64-1.51 (m, 4H), 1.15-1.02 (m, 3H); LCMS (ESI, M+1): m/z=739.6.

### Example 341

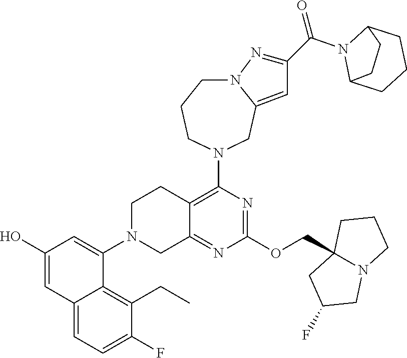 [SMILES: CCc1c(F)ccc2cc(O)cc(N3CCc4c(nc(OC[C@@]56CCCN5C[C@H](F)C6)nc4N4CCCn5nc(C(=O)N6C7CCCC6CC7)cc5C4)C3)c12]

### 8-azabicyclo[3.2.1]octan-8-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

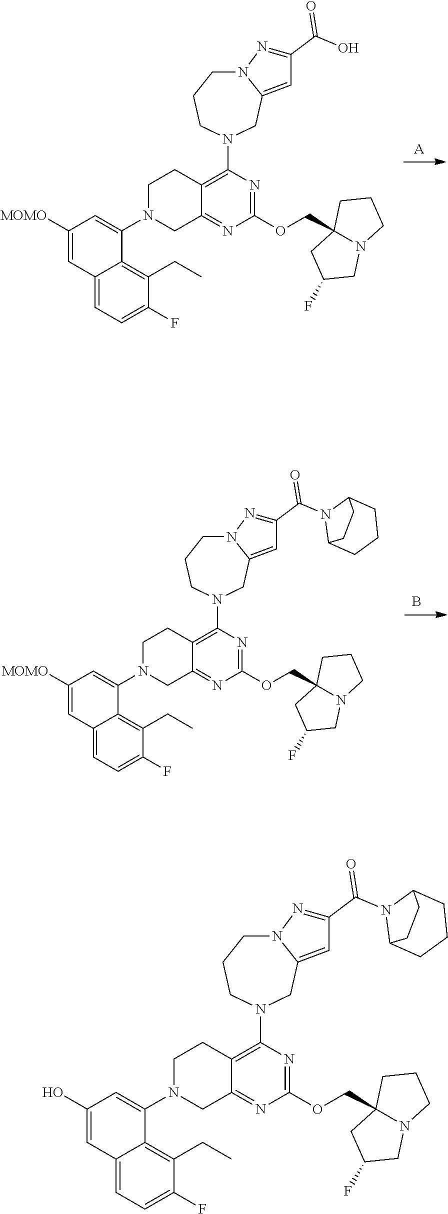

Synthesized according to Example 233 except that HCl instead of TFA was used in the last step. The title compound was obtained as off-white solid; 1H NMR (400 MHz, METHANOL-d4) δ=7.50 (dd, J=6.4, 8.4 Hz, 1H), 7.13 (t, J=9.2 Hz, 1H), 6.96 (br s, 2H), 6.65 (s, 1H), 5.35-5.16 (m, 1H), 5.13 (br d, J=2.8 Hz, 1H), 5.08-4.94 (m, 1H), 4.72 (br s, 1H), 4.54 (br s, 2H), 4.30-4.15 (m, 1H), 4.13-3.96 (m, 4H), 3.73-3.61 (m, 1H), 3.53 (br d, J=10.8 Hz, 1H), 3.46-3.35 (m, 2H), 3.23-3.17 (m, 3H), 3.13 (br s, 2H), 3,00-2,93 (m, 1H), 2.72 (br d, J=14.4 Hz, 1H), 2.40-2.12 (m, 3H), 2.11-1.91 (m, 7H), 1.90-1.76 (m, 6H), 1.67-1.53 (m, 3H), 1.15-1.07 (m, 3H); LCMS (ESI, M+1): m/z=753.4.

### Example 342

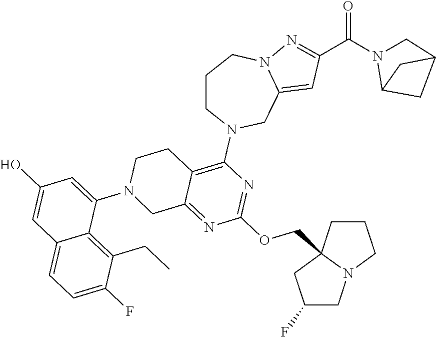 [SMILES: CCc1c(F)ccc2cc(O)cc(N3CCc4c(nc(OC[C@@]56CCCN5C[C@H](F)C6)nc4N4CCCn5nc(C(=O)N6CC7CC6C7)cc5C4)C3)c12]

### 2-azabicyclo[2.1.1]hexan-2-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

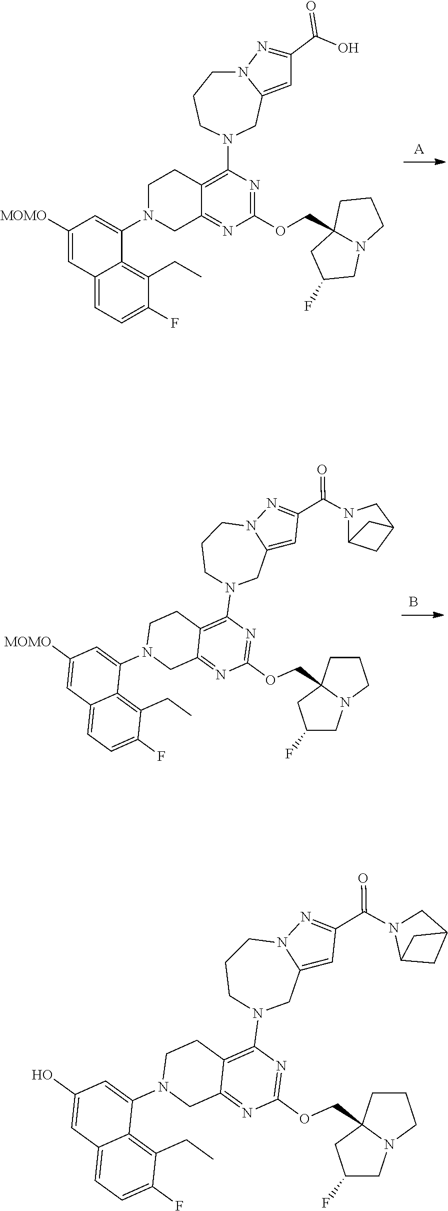

Synthesized according to Example 233 except that HCl instead of TFA was used in the last step. The title compound was obtained as white solid (FA salt). 1H NMR (400 MHz, METHANOL-d4) δ=7.51 (dd, J=6.0, 8.8 Hz, 1H), 7.14 (t, J=9.2 Hz, 1H), 6.97 (s, 2H), 6.70 (d, J=16.4 Hz, 1H), 5.41-5.18 (m, 1H), 5.06-4.94 (m, 1H), 4.90-4.85 (m, 1H), 4.66-4.45 (m, 3H), 4.27-4.00 (m, 5H), 3.96-3.84 (m, 1H), 3.67 (br d, J=17.6 Hz, 1H), 3.54 (br s, 2H), 3.38 (br d, J=7.2 Hz, 2H), 3.28-3.16 (m, 4H), 3.10-3.01 (m, 1H), 2.95 (br d, J=3.6 Hz, 1H), 2.73 (br d, J=13.6 Hz, 1H), 2.46-2.21 (m, 3H), 2.20-2.01 (m, 6H), 2.00-1.82 (m, 2H), 1.52-1.39 (m, 2H), 1.11 (br t, J=7.2 Hz, 3H); LCMS (ESI, M+1): m/z=725.4.

### Example 343

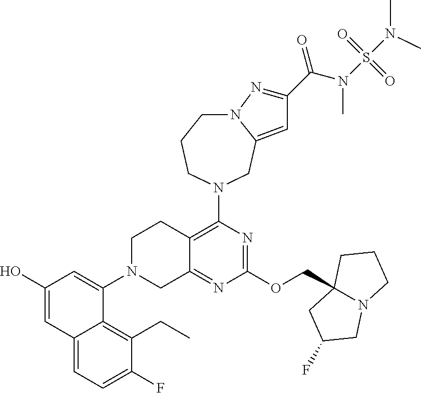 [SMILES: CCc1c(F)ccc2cc(O)cc(N3CCc4c(nc(OC[C@@]56CCCN5C[C@H](F)C6)nc4N4CCCn5nc(C(=O)N(C)S(=O)(=O)N(C)C)cc5C4)C3)c12]

### 7-(8-ethyl-7-fluoro-3-hydroxy-1-naphthyl)-2-(((2R,7aS)-2-fluorotetrahydro-1H-pyrrolizin-7a(5H)-yl)methoxy)-4-(2-((dimethylsulfamoyl)methylamino)methyl-7,8-dihydro-4H-pyrazolo[1,5-a][1,4]diazepin-5(6H)-yl)-6,8-dihydro-5H-pyrido[3,4-d]pyrimidine

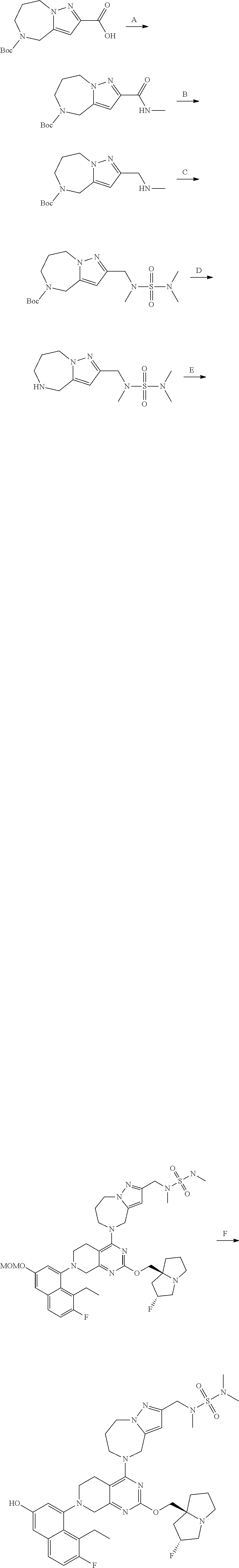

Step A. tert-butyl 2-(methylcarbamoyl)-7,8-dihydro-4H-pyrazolo[1,5-a][1,4]diazepine-5(6H)-carboxylate: To a solution of 5-(tert-butoxycarbonyl)-5,6,7,8-tetrahydro-4H-pyrazolo[1,5-a][1,4]diazepine-2-carboxylic acid (200 mg, 1.0 equiv), DIEA (276 mg, 3.0 equiv) and HATU (541 mg, 2.0 equiv) in DMF (2 mL) was added methanamine hydrochloride (96.0 mg, 2.0 equiv). The reaction was stirred at 20° C. for 2 hours. The mixture was filtered and purified with reversed phase flash (0.1% FA condition) to afford the title compound (200 mg, 93% yield) as white solid.

Step B. tert-butyl 2-((methylamino)methyl)-7,8-dihydro-4H-pyrazolo[1,5-a][1,4]diazepine-5(6H)-carboxylate: To a solution of tert-butyl 2-(methylcarbamoyl)-7,8-dihydro-4H-pyrazolo[1,5-a][1,4]diazepine-5(6H)-carboxylate (180 mg, 1.0 equiv) in THF (1 mL) was added BH3Me2S (10 M, 0.6 mL, 10 equiv) at 0° C. The reaction was stirred at 60° C. for 12 hours. The mixture was quenched with HCl (2 M, 2 mL) and stirred at 60° C. for 0.5 hours. The mixture was concentrated and purified by reversed phase flash (0.1% FA condition) to afford the title compound (240 mg, crude) as white solid; LCMS (ESI, M+1): m/z=280.9.

Step C. tert-butyl 2-(((N,N-dimethylsulfamoyl)(methyl)amino)methyl)-7,8-dihydro-4H-pyrazolo[1,5-a][1,4]diazepine-5(6H)-carboxylate: To a solution of tert-butyl 2-((methylamino)methyl)-7,8-dihydro-4H-pyrazolo[1,5-a][1,4]diazepine-5(6H)-carboxylate (225 mg, 1.0 equiv) in DCM (2 mL) were added TEA (244 mg, 3.0 equiv) and dimethylsulfamoyl chloride (230 mg, 2.0 equiv). The reaction was stirred at 20° C. for 3 hours. The mixture was concentrated and purified with reversed phase flash (0.1% FA condition) to afford the title compound (60.0 mg, 19% yield) as white solid; LCMS (ESI, M+1): m/z=388.1.

Step D. N-(dimethylsulfamoyl)-N-methyl-1-(5,6,7,8-tetrahydro-4H-pyrazolo[1,5-a][1,4]diazepin-2-yl)methanamine: A solution of tert-butyl 2-[[dimethylsulfamoyl(methyl)amino]methyl]-4,6,7,8-tetrahydropyrazolo[1,5-a][1,4]diazepine-5-carboxylate (50.0 mg, 1.0 equiv) in HCl•dioxane (4 M, 0.5 mL, 15 equiv) was stirred at 20° C. for 1 hour. The reaction was concentrated to afford the title compound (50.0 mg, crude, HCl) as white solid.

The last two steps were performed according to Example 248. The title compound was obtained as off-white solid (FA salt). 1H NMR (400 MHz, METHANOL-d4) δ=7.51 (dd, J=5.8, 9.2 Hz, 1H), 7.14 (t, J=9.2 Hz, 1H), 6.97 (s, 2H), 6.29 (s, 1H), 5.44-5.21 (m, 1H), 5.03-4.91 (m, 1H), 4.85 (br d, J=6.4 Hz, 1H), 4.50-4.42 (m, 2H), 4.28-4.22 (m, 2H), 4.21-4.12 (m, 3H), 4.10-4.02 (m, 2H), 3.68 (br dd, J=2.4, 17.6 Hz, 1H), 3.56-3.47 (m, 1H), 3.45-3.33 (m, 4H), 3.24-3.12 (m, 3H), 3.11-3.05 (m, 1H), 2.78 (s, 6H), 2.73 (s, 3H), 2.41-2.29 (m, 1H), 2.29-2.21 (m, 2H), 2.20-2.02 (m, 4H), 2.01-1.86 (m, 2H), 1.12 (t, J=7.2 Hz, 3H); LCMS (ESI, M+1): m/z=766.5.

### Example 344

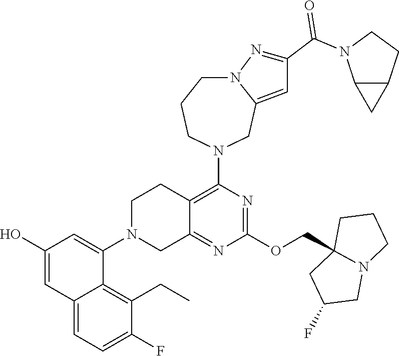 [SMILES: CCc1c(F)ccc2cc(O)cc(N3CCc4c(nc(OC[C@@]56CCCN5C[C@H](F)C6)nc4N4CCCn5nc(C(=O)N6CCC7CC76)cc5C4)C3)c12]

### 2-azabicyclo[3.1.0]hexan-2-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

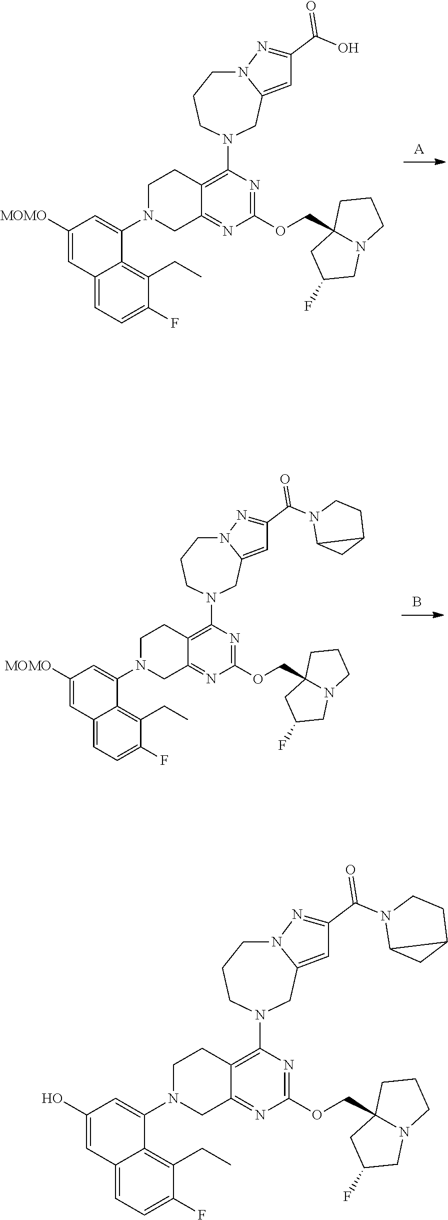

Synthesized according to Example 233 except that HCl instead of TFA was used in the last step. The title compound was obtained as white solid (FA salt). 1H NMR (400 MHz, METHANOL-d4) δ=7.51 (dd, J=6.0, 9.2 Hz, 1H), 7.14 (t, J=9.2 Hz, 1H), 6.97 (s, 2H), 6.77-6.64 (m, 1H), 5.4-5.21 (m, 1H), 5.09-4.97 (m, 1H), 4.88 (br d, J=3.2 Hz, 1H), 4.58-4.50 (m, 2H), 4.48-4.29 (m, 2H), 4.24-3.91 (m, 6H), 3.67 (br d, J=17.2 Hz, 1H), 3.59-3.50 (m, 1H), 3.49-3.36 (m, 3H), 3.15 (br s, 4H), 3.10 (br d, J=6.4 Hz, 1H), 2.79-2.69 (m, 1H), 2.39-2.16 (m, 4H), 2.15-1.96 (m, 5H), 1.95-1.86 (m, 1H), 1.83-1.67 (m, 1H), 1.11 (br t, J=6.8 Hz, 3H), 0.96-0.77 (m, 1H), 0.74-0.60 (m, 1H); LCMS (ESI, M+1): m/z=725.4.

### Example 345

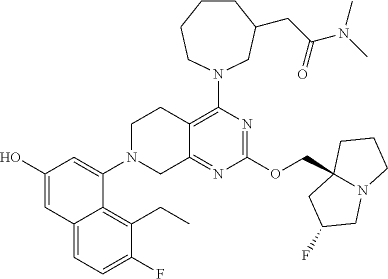 [SMILES: CCc1c(F)ccc2cc(O)cc(N3CCc4c(nc(OC[C@@]56CCCN5C[C@H](F)C6)nc4N4CCCCC(CC(=O)N(C)C)C4)C3)c12]

### 2-(1-(7-(8-ethyl-7-fluoro-3-hydroxynaphthalen-1-yl)-2-(((2R,7aS)-2-fluorohexahydro-1H-pyrrolizin-7a-yl)methoxy)-5, 6,7,8-tetrahydropyrido[3,4-d]pyrimidin-4-yl)azepan-3-yl)-N,N-dimethylacetamide

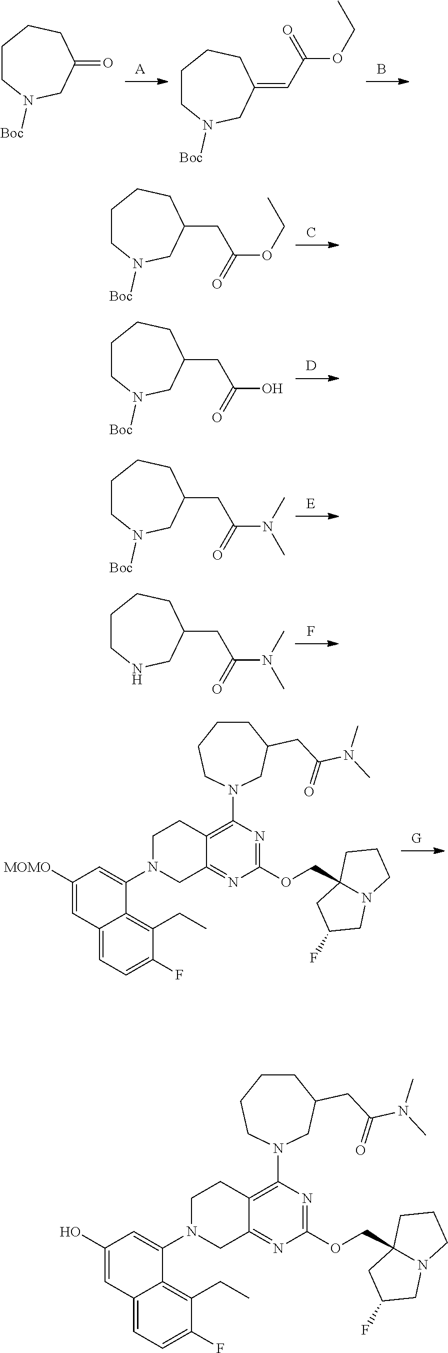

Step A. (E)-tert-butyl 3-(2-ethoxy-2-oxoethylidene)azepane-1-carboxylate: To a solution of ethyl 2-diethoxyphosphorylacetate (23.6 g, 1.5 equiv) in THE (100 mL) was added NaH (4.78 g, 60% purity, 1.7 equiv) at 0° C. To the mixture was added tert-butyl 3-oxoazepane-1-carboxylate (15.0 g, 1.0 equiv) dropwise at 0° C. for 5 minutes. The mixture was stirred at 15° C. for 1 hour. The mixture was quenched with water (50 mL) slowly and extracted with ethyl acetate (2×30 mL). The organic layer was washed with brine (20 mL) and dried over Na₂SO₄. The solvent was concentrated and purified by reversed phase flash chromatography [C18, 0.1% formic acid condition] to afford the title compound (16.0 g, 79% yield) as yellow oil; 1H NMR (400 MHz, METHANOL-d4) δ=6.49-5.69 (m, 1H), 4.22-4.01 (m, 3H), 3.61 (br s, 1H), 3.41-3.22 (m, 1H), 3.00 (s, 1H), 2.84-2.68 (m, 1H), 2.28-2.16 (m, 1H), 1.82-1.61 (m, 4H), 1.57-1.39 (m, 9H), 1.29-1.22 (m, 3H); LCMS (ESI, M-100+1): m/z=184.2.

Step B. tert-butyl 3-(2-ethoxy-2-oxoethyl)azepane-1-carboxylate: To a mixture of Pd/C (1.00 g, 10% purity, 1.0 equiv) in MeOH (40 mL) was added (E)-tert-butyl 3-(2-ethoxy-2-oxoethylidene)azepane-1-carboxylate (5.00 g, 1.0 equiv) slowly under N2 atmosphere. The suspension was degassed and purged with H2 3 times. The mixture was stirred under H2 (15 psi) at 15° C. for 10 hours. The mixture was filtered and concentrated to afford the title compound (5.00 g, crude) as white oil.

Step C. 2-(1-(tert-butoxycarbonyl)azepan-3-yl)acetic acid: To a solution of tert-butyl 3-(2-ethoxy-2-oxo-ethyl)azepane-1-carboxylate (1.00 g, 1.0 equiv) in THE (10 mL) was added LiOH H₂O (588 mg, 4.0 equiv) at 0° C. The mixture was stirred at 15° C. for 3 hours. The mixture was diluted with water (10 mL) and extracted with MTBE (2×10 mL). The pH of the water layer was adjusted to 2-3 with 1 M of HCl. The mixture was extracted with ethyl acetate (2×10 mL). The organic layer was washed with brine (10 mL) and dried over Na₂SO₄. The solvent was concentrated to afford the title compound (800 mg, 89% yield) as colorless oil; LCMS (ESI, M-100+1): m/z=158.2

Step D. tert-butyl 3-(2-(dimethylamino)-2-oxoethyl)azepane-1-carboxylate: To a solution of 2-(1-(tert-butoxycarbonyl)azepan-3-yl)acetic acid (1.50 g, 1.0 equiv) in DMF (10 mL) were added Me2NH (2 M, 8.7 mL, 3.0 equiv), TEA (4.13 g, 7.0 equiv), EDCI (2.23 g, 2.0 equiv) and HOBt (1.58 g, 2.0 equiv) at 0° C. The mixture was stirred at 15° C. for 10 hours. The mixture was diluted with water (10 mL) and extracted with ethyl acetate (2×10 mL). The organic layer was washed with brine (10 mL) and dried over Na₂SO₄. The solvent was concentrated and purified by reversed phase flash chromatography [C18, 0.1% formic acid condition] to afford the title compound (190 mg, 9.4% yield) as yellow oil; LCMS (ESI, M+1): m/z=285.1.

Step E. 2-(azepan-3-yl)-N,N-dimethylacetamide: To a solution of tert-butyl 3-[2-(dimethylamino)-2-oxo-ethyl]azepane-1-carboxylate (170 mg, 1.0 equiv) in MeOH (1 mL) was added HCl•MeOH (4 M, 1.62 mL, 11 equiv) at 0° C. The mixture was concentrated to dryness. To the residue was added NaHCO₃ (200 mg) in MeOH (5 mL). The mixture was stirred for 0.5 hours. The mixture was filtered and concentrated to afford the title compound (150 mg, crude) as white oil.

The last two steps were performed according to Example 248. The title compound was obtained as white solid; 1H NMR (400 MHz, METHANOL-d4) δ=7.567.42 (m, 1H), 7.19-7.08 (m, 1H), 7.04-6.90 (m, 2H), 5.39-5.09 (m, 1H), 4.35-3.96 (m, 5H), 3.77-3.45 (m, 3H), 3.42-3.34 (m, 2H), 3.18 (br d, J=5.6 Hz, 3H), 3.14 (br d, J=4.4 Hz, 2H), 3.09-3.05 (m, 3H), 3.03-2.88 (m, 4H), 2.82-2.52 (m, 2H), 2.48-2.25 (m, 3H), 2.23-2.03 (m, 3H), 2.00-1.65 (m, 7H), 1.56-1.28 (m, 2H), 1.15-1.05 (m, 3H); LCMS (ESI, M+1): m/z=663.5.

### Example 346

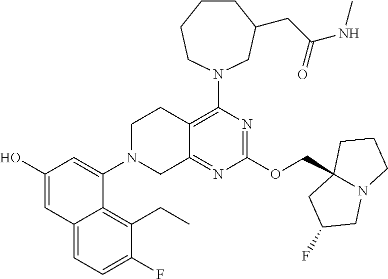 [SMILES: CCc1c(F)ccc2cc(O)cc(N3CCc4c(nc(OC[C@@]56CCCN5C[C@H](F)C6)nc4N4CCCCC(CC(=O)NC)C4)C3)c12]

### 2-(1-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)azepan-3-yl)-N-methylacetamide

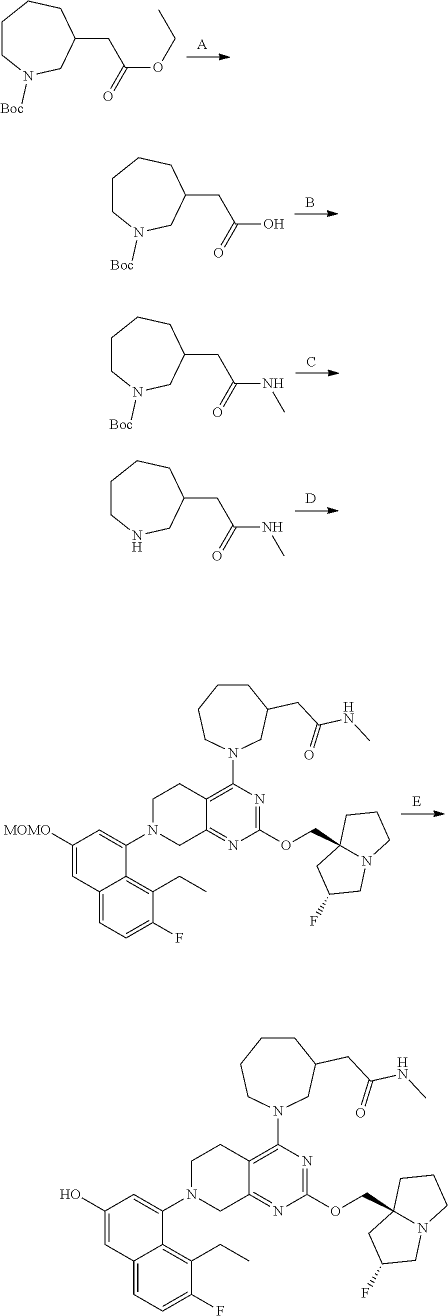

Step A. 2-(1-(tert-butoxycarbonyl)azepan-3-yl)acetic acid: To a mixture of Pd/C (1.00 g, 10% purity, 1.0 equiv) in MeOH (30 mL) was added tert-butyl 3-(2-ethoxy-2-oxoethyl)azepane-1-carboxylate (4.00 g, 1.0 equiv) slowly under N2 atmosphere. The suspension was degassed and purged with H2 3 times. The mixture was stirred under H2 (15 psi) at 15° C. for 10 hours. The mixture was filtered by MeOH (20 mL) and concentrated to afford the title compound (3.30 g, crude) as white oil.

Step B. tert-butyl 3-(2-(methylamino)-2-oxoethyl)azepane-1-carboxylate: To a solution of 2-(1-(tert-butoxycarbonyl)azepan-3-yl)acetic acid (1.50 g, 1.0 equiv) in DMF (10 mL) were added methanamine;hydrochloride (1.18 g, 3.0 equiv), TEA (4.13 g, 7.0 equiv), EDCI (2.24 g, 2.0 equiv) and HOBt (1.58 g, 2.0 equiv) at 0° C. The mixture was stirred at 25° C. for 10 hours. The mixture was diluted with water (10 mL) and extracted with ethyl acetate (2×10 mL). The organic layer was washed with brine (10 mL) and dried over Na₂SO₄. The solvent was concentrated and purified by reversed phase flash chromatography [C18, 0.1% formic acid condition] to afford the title compound (600 mg, 35% yield) as yellow oil; LCMS (ESI, M-100+1): m/z=171.3

Step C. 2-(azepan-3-yl)-N-methylacetamide: To a solution of tert-butyl 3-(2-(methylamino)-2-oxoethyl)azepane-1-carboxylate (250 mg, 1.0 equiv) in MeOH (2 mL) was added HCl•MeOH (4 M, 3 mL, 13 equiv) at 0° C. The mixture was stirred at 0° C. for 1 hour. The mixture was concentrated to dryness. To the residue was added NaHCO₃ (200 mg) in MeOH (5 mL). The mixture was stirred at 25° C. for 0.5 hours. The mixture was filtered and concentrated to afford the title compound (200 mg, crude) as white oil.

The last two steps were performed according to Example 248. The title compound was obtained as pink solid; 1H NMR (400 MHz, METHANOL-d4) 8=7.50 (ddd, J=2.8, 6.0, 8.8 Hz, 1H), 7.13 (t, J=9.2 Hz, 1H), 7.05-6.91 (m, 2H), 5.38-5.16 (m, 1H), 4.43-3.95 (m, 5H), 3.75-3.45 (m, 3H), 3.44-3.35 (m, 2H), 3.25-3.18 (m, 2H), 3.17-3.05 (m, 3H), 3.03-2.93 (m, 1H), 2.78 (br d, J=14.4 Hz, 1H), 2.73-2.67 (m, 3H), 2.63-2.47 (m, 1H), 2.37-2.05 (m, 6H), 2.03-1.62 (m, 7H), 1.50-1.29 (m, 2H), 1.10 (t, J=7.2 Hz, 3H); LCMS (ESI, M+1): m/z=649.5.

### Example 347

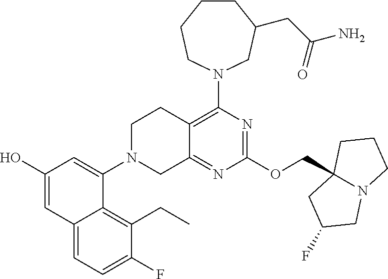 [SMILES: CCc1c(F)ccc2cc(O)cc(N3CCc4c(nc(OC[C@@]56CCCN5C[C@H](F)C6)nc4N4CCCCC(CC(N)=O)C4)C3)c12]

### 2-(1-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)azepan-3-yl)acetamide

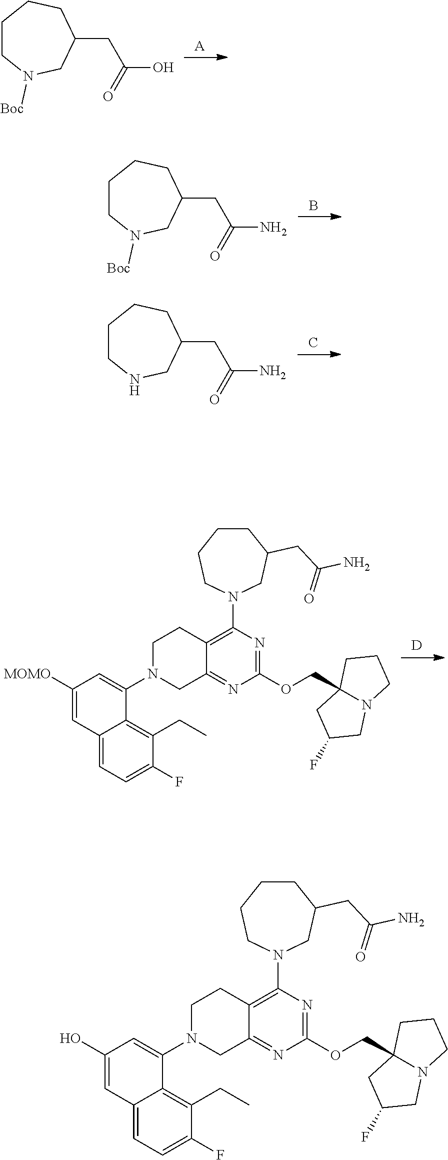

Step A. tert-butyl 3-(2-amino-2-oxoethyl)azepane-1-carboxylate: To a solution of 2-(1-(tert-butoxycarbonyl)azepan-3-yl)acetic acid (800 mg, 1.0 equiv) in DMF (8 mL) were added NH₄Cl (832 mg, 5.0 equiv), TEA (3.15 g, 10 equiv), EDCI (715 mg, 1.2 equiv) and HOBt (840 mg, 2.0 equiv) at 0° C. The mixture was stirred at 25° C. for 10 hours. The mixture was diluted with water (10 mL) and extracted with ethyl acetate (2×10 mL). The organic layer was washed with brine (10 mL) and dried over Na₂SO₄. The solvent was concentrated and purified by reversed phase flash chromatography [C18, 0.1% formic acid condition] to afford the title compound (200 mg, 25% yield) as white oil; LCMS (ESI, M-100+1): m/z=157.2.

Step B: 2-(azepan-3-yl)acetamide: To a solution of tert-butyl 3-(2-amino-2-oxoethyl)azepane-1-carboxylate (200 mg, 1.0 equiv) in MeOH (1.5 mL) was added HCl-MeOH (4 M, 2.5 mL, 13 equiv) at 0° C. The mixture was stirred at 0° C. for 1 hour. The mixture was concentrated to dryness. To the residue was added NaHCO₃ (200 mg) in MeOH (5 mL). The mixture was stirred at 25° C. for 0.5 hours. The mixture was filtered and concentrated to afford the title compound (190 mg, crude) as white oil.

The last two steps were performed according to Example 248. The title compound was obtained as yellow solid; 1H NMR (400 MHz, METHANOL-d4) δ=7.50 (ddd, J=3.6, 5.6, 9.2 Hz, 1H), 7.20-7.09 (m, 1H), 7.04-6.92 (m, 2H), 5.39-5.16 (m, 1H), 4.54-3.92 (m, 5H), 3.76-3.46 (m, 3H), 3.44-3.36 (m, 2H), 3.27-3.19 (m, 2H), 3.18-3.05 (m, 3H), 2.98 (br d, J=6.0 Hz, 1H), 2.83-2.67 (m, 1H), 2.56 (br dd, J=5.2, 6.4 Hz, 1H), 2.412.04 (m, 6H), 2.03-1.63 (m, 7H), 1.49-1.29 (m, 2H), 1.11 (br t, J=7.2 Hz, 3H); LCMS (ESI, M+1): m/z=635.5.

### Example 348

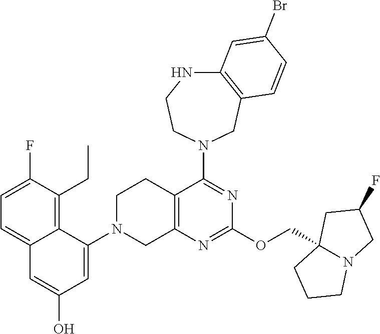 [SMILES: CCc1c(F)ccc2cc(O)cc(N3CCc4c(nc(OC[C@@]56CCCN5C[C@H](F)C6)nc4N4CCNc5cc(Br)ccc5C4)C3)c12]

### 4-(4-(8-bromo-1,2,3,5-tetrahydro-4H-benzo[e][1,4]diazepin-4-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-ol

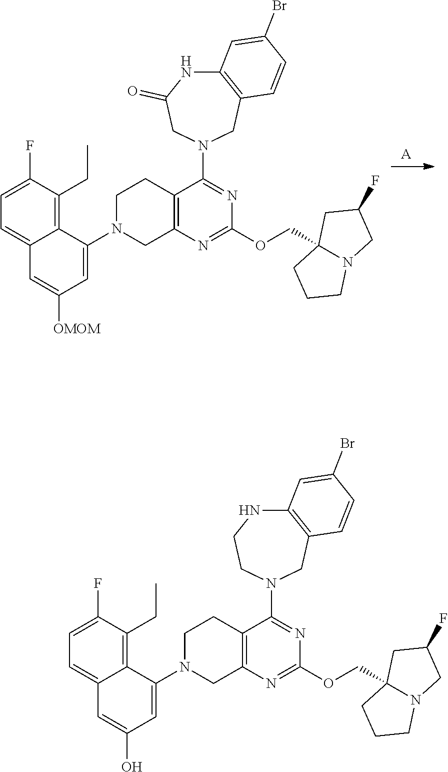

Step A 4-(4-(8-bromo-1,2,3,5-tetrahydro-4H-benzo[e][1,4]diazepin-4-yl)-2-(((2R,7aS)-2-fluorotetrahydro-1H-pyrrolizin-7a(5H)-yl)methoxy)-5, 8-dihydropyrido[3,4-d]pyrimidin-7(6H)-yl)-5-ethyl-6-fluoronaphthalen-2-ol: A mixture of 8-bromo-4-(7-(8-ethyl-7-fluoro-3-(methoxymethoxy) naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,4,5-tetrahydro-2H-benzo[e][1,4]diazepin-2-one (20.0 mg, 1.00 equiv) and borane dimethyl sulfide complex (5.24 L, 2.00 equiv) in THE (1.00 mL) was stirred at 20° C. for 12 hours. The reaction was quenched with HCl (aq., 3.00 mL) at 0° C. and stirred at 50° C. for 3 hours. The reaction was diluted with water (10 mL) and extracted with DCM/MeOH=10/1 (10.00 mL×3). The combined organic layers were washed with brine (20 mL) and dried over anhydrous sodium sulfate. The reaction was filtered and concentrated under reduced pressure to give a residue. The residue was purified with prep-HPLC [column: Phenomenex luna C18 150×25 mm×10 μm; mobile phase: [water(FA)-ACN]; B %: 27%-57%, 10 minutes] to afford the title compound (4.07 mg) as yellow solid (FA salt). 1H NMR (400 MHz, METHANOL-d4) δ=8.59-8.46 (m, 1H), 7.51 (br dd, J=5.6, 8.8 Hz, 1H), 7.19-7.06 (m, 2H), 7.04-6.94 (m, 2H), 6.94-6.82 (m, 2H), 5.50-5.14 (m, 1H), 4.86-4.57 (m, 4H), 4.17-3.81 (m, 5H), 3.69-3.37 (m, 5H), 3.00-2.95 (m, 1H), 3.23-2.95 (m, 3H), 2.76-2.62 (m, 1H), 2.42-1.71 (m, 7H), 1.12 (br t, J=7.2 Hz, 3H); LCMS (ESI, M+1): m/z=705.3.

### Example 349

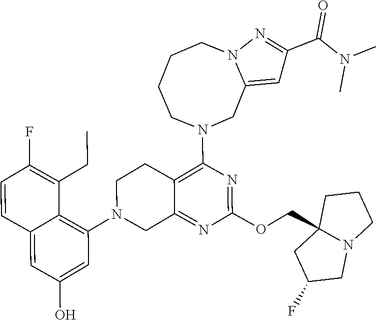 [SMILES: CCc1c(F)ccc2cc(O)cc(N3CCc4c(nc(OC[C@@]56CCCN5C[C@H](F)C6)nc4N4CCCCn5nc(C(=O)N(C)C)cc5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-4,5,6,7,8,9-hexahydropyrazolo[1,5-a][1,4]diazocine-2-carboxamide

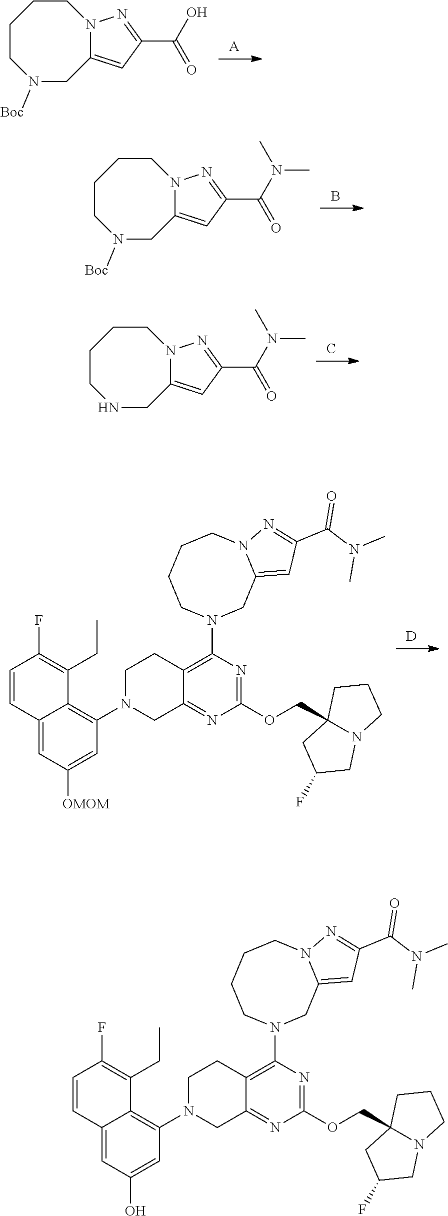

Step A tert-butyl 2-(dimethylcarbamoyl)-6,7,8,9-tetrahydropyrazolo[1,5-a][1,4]diazocine-5(4H)-carboxylate: To a solution of 5-(tert-butoxycarbonyl)-4,5,6,7,8,9-hexahydropyrazolo[1,5-a][1,4]diazocine-2-carboxylic acid (300 mg, 1.0 equiv) in DMF (5.0 mL) were added DIEA (1.3 g, 10 equiv), HATU (1.2 g, 3.0 equiv) and methanamine hydrochloride (172 mg, 1.5 equiv). The reaction was stirred at 25° C. for 1 hour. The mixture was diluted with ethyl acetate (100 mL) and filtered. The filtrate was washed with brine (50 mL×3). The organic phase was dried over anhydrous sodium sulfate, concentrated, and purified by column chromatography (SiO2, petroleum ether/ethyl acetate=2/1 to I/O) to afford the title compound (210 mg, 64.1% yield) as a colorless oil; LCMS (ESI, M+1): m/z=323.2.

Step B N,N-dimethyl-4,5,6,7,8,9-hexahydropyrazolo[1,5-a][1,4]diazocine-2-carboxamide: A solution of tert-butyl 2-(dimethylcarbamoyl)-6,7,8,9-tetrahydropyrazolo[1,5-a][1,4]diazocine-5(4H)-carboxylate (190 mg, 1.0 equiv) in MeCN (1.00 mL) and HCl/dioxane (4 M, 1.0 mL) was stirred at 25° C. for 1 hour. The reaction was concentrated to afford the title compound (120 mg, crude), LCMS (ESI, M+1): m/z=223.2.

The last two steps were performed according to Example 248. The title compound was obtained as pink solid (FA salt). 1H NMR (400 MHz, METHANOL-d4) δ=7.50 (dd, J=6.0, 10.0 Hz, 1H), 7.14 (t, J=10.0 Hz, 1H), 7.01-6.92 (m, 2H), 6.56 (d, J=1.2 Hz, 1H), 5.37-5.20 (m, 1H), 5.06-4.93 (m, 3H), 4.49-4.40 (m, 2H), 4.15-4.09 (m, 1H), 4.08-3.98 (m, 2H), 3.91-3.83 (m, 1H), 3.82-3.73 (m, 1H), 3.69 (br d, J=18.0 Hz, 1H), 3.52-3.46 (m, 1H), 3.40 (br d, J=6.8 Hz, 2H), 3.35-3.31 (m, 8H), 3.26-3.12 (m, 4H), 3.09 (s, 3H), 3.05-3.00 (m, 1H), 2.75-2.64 (m, 1H), 2.35-2.15 (m, 2H), 2.12-2.03 (m, 1H), 2.02-1.91 (m, 4H), 1.89-1.78 (m, 2H), 1.78-1.66 (m, 1H), 1.11 (t, J=7.2 Hz, 3H); LCMS (ESI, M+1): m/z=701.5.

### Example 350

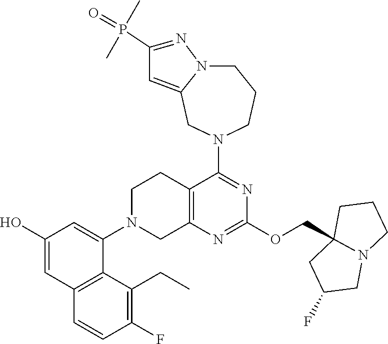 [SMILES: CCc1c(F)ccc2cc(O)cc(N3CCc4c(nc(OC[C@@]56CCCN5C[C@H](F)C6)nc4N4CCCn5nc(P(C)(C)=O)cc5C4)C3)c12]

### (5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)dimethylphosphine oxide

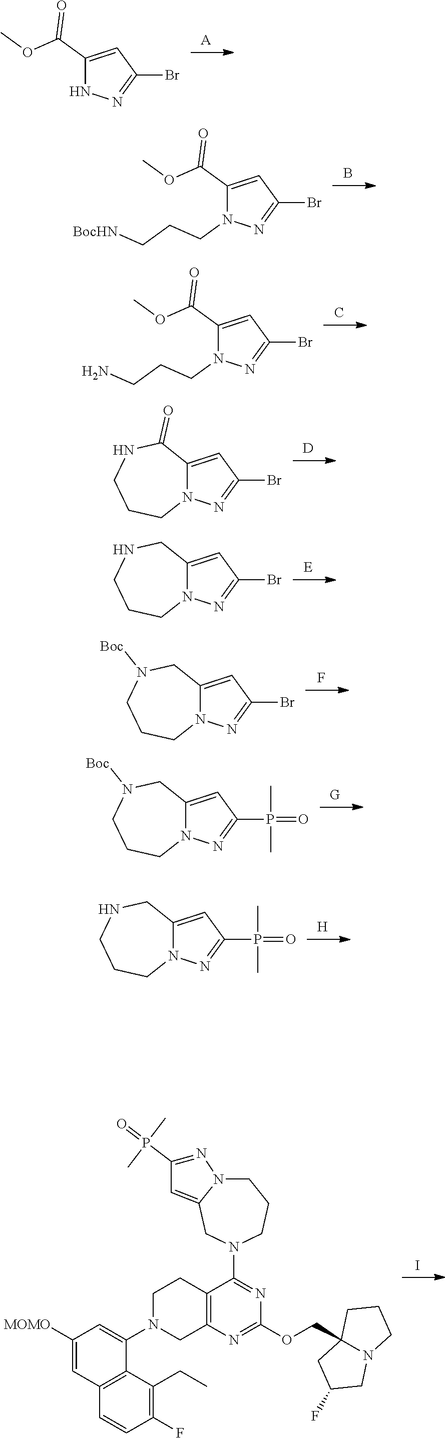

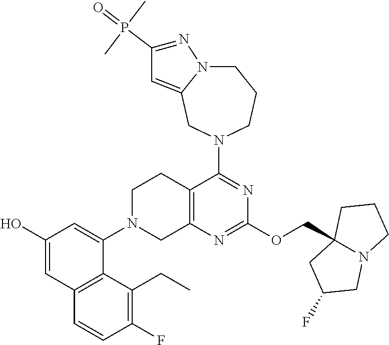 [SMILES: CCc1c(F)ccc2cc(O)cc(N3CCc4c(nc(OC[C@@]56CCCN5C[C@H](F)C6)nc4N4CCCn5nc(P(C)(C)=O)cc5C4)C3)c12]

Step A. methyl 3-bromo-1-(3-((tert-butoxycarbonyl)amino)propyl)-1H-pyrazole-5-carboxylate: To a solution of methyl 3-bromo-1H-pyrazole-5-carboxylate (1.0 g, 1.0 equiv) in ACN (10.0 mL) were added Cs2CO₃ (3.2 g, 2.0 equiv) and tert-butyl N-(3-bromopropyl)carbamate (1.4 g, 1.2 equiv). The reaction was stirred at 15° C. for 2 hours. The mixture was filtered and purified by flash silica gel chromatography (ISCO®; 12 g SepaFlash® Silica Flash Column, Eluent of 0-30% Ethyl acetate/Petroleum ether gradient @36 mL/min) to afford the title compound (1.3 g, 68.3% yield, 95% purity) as a colorless clear liquid. 1H NMR (400 MHz, CHLOROFORM-d) δ=6.81 (s, 1H), 4.88 (br s, 1H), 4.67-4.57 (m, 2H), 3.96-3.85 (m, 3H), 3.10 (br d, J=5.6 Hz, 2H), 2.09-1.98 (m, 3H), 1.45 (s, 1OH)

Step B. methyl 1-(3-aminopropyl)-3-bromo-1H-pyrazole-5-carboxylate: To a solution methyl 3-bromo-1-(3-((tert-butoxycarbonyl)amino)propyl)-1H-pyrazole-5-carboxylate (1.3 g,1.0 equiv) in dioxane (5.0 mL) was added HCl•dioxane (4 M, 15 mL, 17.1 equiv). The reaction was stirred at 20° C. for 12 hours. The mixture was concentrated under reduced pressure to afford the title compound (1.1 g, HCl) as a white solid.

Step C. 2-bromo-5,6,7,8-tetrahydro-4H-pyrazolo[1,5-a][1,4]diazepin-4-one: A solution of methyl 1-(3-aminopropyl)-3-bromo-1H-pyrazole-5-carboxylate hydrochloride (1.1 g, 1.0 equiv) in saturated Na₂CO₃ (20 mL) solution was stirred at 20° C. for 12 hours. The reaction was diluted with H₂O (30 mL) and extracted with DCM 100 mL (20 mL×5). The combined organic layers were washed with brine (30 mL), dried over Na₂SO₄, filtered, concentrated, and triturated with DCM/PE at 20° C. for 5 minutes to afford the title compound (690 mg, 79.3% yield). 1H NMR (400 MHz, CHLOROFORM-d) δ=7.00-6.93 (m, 1H), 6.84 (s, 1H), 4.49 (t, J=6.8 Hz, 2H), 3.43-3.38 (m, 3H), 2.32-2.22 (m, 3H); LCMS (ESI, M+1): m/z=232.0.

Step D. 2-bromo-5,6,7,8-tetrahydro-4H-pyrazolo[1,5-a][1,4]diazepine: A mixture of 2-bromo-5,6,7,8-tetrahydro-4H-pyrazolo[1,5-a][1,4]diazepin-4-one (610 mg, 1.0 equiv) in THF (5.0 mL) was degassed and purged with N2 3 times. BH3Me2S (10 M, 3.0 equiv) was added at 0° C. and the reaction was stirred at 20° C. for 30 minutes then at 60° C. for 12 hours. The mixture was concentrated under reduced pressure to afford the title compound (650 mg, 97% yield, HCl) as a white solid. 1H NMR (400 MHz, DMSO-d6) δ=6.55 (s, 1H), 4.48-4.34 (m, 5H), 3.38 (br dd, J=6.0, 12.0 Hz, 3H), 2.01 (br d, J=4.0 Hz, 2H).

Step E. tert-butyl 2-bromo-7,8-dihydro-4H-pyrazolo[1,5-a][1,4]diazepine-5(6H)-carboxylate: To a solution of 2-bromo-5,6,7,8-tetrahydro-4H-pyrazolo[1,5-a][1,4]diazepine (650 mg, 1.0 equiv, HCl) in DCM (8 mL) were added TEA (520 mg, 716 μL 2.0 equiv) and Boc20 (842 mg, 1.5 equiv). The mixture was stirred at 25° C. for 2 hours. The reaction was concentrated and purified by flash silica gel chromatography (ISCO®; 12 g SepaFlash® Silica Flash Column, Eluent of 0˜30% Ethyl acetate/Petroleum ether gradient @30 mL/min) to afford the title compound (250 mg, 28.9% yield, 94.0% purity) as a white solid. 1H NMR (400 MHz, CHLOROFORM-d) δ=6.32-6.12 (m, 1H), 4.48-4.35 (m, 4H), 3.78-3.65 (m, 2H), 1.91 (br d, J=4.4 Hz, 2H), 1.51-1.40 (m, 9H); LCMS (ESI, M+1): m/z=318.0.

Step F. tert-butyl 2-(dimethylphosphoryl)-7,8-dihydro-4H-pyrazolo[1,5-a][1,4]diazepine-5(6H)-carboxylate: A mixture of tert-butyl 2-bromo-7,8-dihydro-4H-pyrazolo[1,5-a][1,4]diazepine-5(6H)-carboxylate (250 mg, 1.0 equiv), methylphosphonoylmethane (74.1 mg, 1.2 equiv), Pd(OAc)₂ (17.7 mg, 0.1 equiv), K3PO4 (201 mg, 1.2 equiv) and (5-diphenylphosphanyl-9,9-dimethyl-xanthen-4-yl)-diphenyl-phosphane (45.7 mg, 1.0 equiv) in DMF (4.0 mL) was degassed and purged with N2 3 times, and then stirred at 140° C. for 12 hours under N2 atmosphere. The mixture was filtered through a pad of celite, and the cake was washed with ethyl acetate. The filtrate was concentrated and purified with prep-HPLC [C18, 0.1% formic acid condition] to afford the title compound (120 mg, 48.4% yield) as a yellow solid. LCMS (ESI, M+1): m/z=314.2.

Step G. dimethyl(5,6,7,8-tetrahydro-4H-pyrazolo[1,5-a][1,4]diazepin-2-yl)phosphine oxide: To a tert-butyl 2-(dimethylphosphoryl)-7,8-dihydro-4H-pyrazolo[1,5-a][1,4]diazepine-5(6H)-carboxylate (120 mg, 1.0 equiv) in dioxane (1.0 mL) was added HCl•dioxane (4 M, 2.4 mL). The reaction was stirred at 20° C. for 2 hours. The reaction was concentrated under reduced pressure to afford the title compound (90 mg, 94.1% yield, HCl) as a white solid. 1H NMR (400 MHz, METHANOL-d4) δ=6.94 (s, 1H), 4.69-4.63 (m, 2H), 4.58 (s, 2H), 3.68 (s, 3H), 3.62-3.58 (m, 2H), 2.23-2.15 (m, 2H), 1.82 (dd, J=1.6, 14.0 Hz, 6H).

The last two steps were performed according to Example 248. The title compound was obtained as white solid (FA salt). 1H NMR (400 MHz, METHANOL-d4) δ=9.17 (s, 1H), 8.57-8.51 (m, 1H), 7.68 (dd, J=6.0, 9.0 Hz, 1H), 7.31 (d, J=2.4 Hz, 1H), 7.26 (t, J=9.2 Hz, 1H), 7.05 (d, J=2.4 Hz, 1H), 6.91 (s, 1H), 5.46-5.37 (m, 1H), 5.36-5.27 (m, 2H), 4.63 (br d, J=5.6 Hz, 2H), 4.45 (br d, J=4.4 Hz, 2H), 4.40-4.30 (m, 2H), 3.37 (br s, 1H), 3.16-3.06 (m, 1H), 2.54-2.37 (m, 4H), 2.36-2.26 (m, 2H), 2.25-2.03 (m, 5H), 2.00-1.93 (m, 1H), 1.81 (s, 3H), 1.78 (s, 3H), 1.38-1.24 (m, 1H), 0.79 (t, J=6.8 Hz, 3H); 19F NMR (376 MHz, methanol-d4) δ=−122, -173; LCMS (ESI, M+1): m/z=692.3.

### Example 351

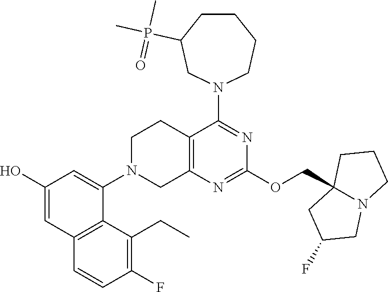 [SMILES: CCc1c(F)ccc2cc(O)cc(N3CCc4c(nc(OC[C@@]56CCCN5C[C@H](F)C6)nc4N4CCCCC(P(C)(C)=O)C4)C3)c12]

### (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azepan-3-yl)dimethylphosphine oxide

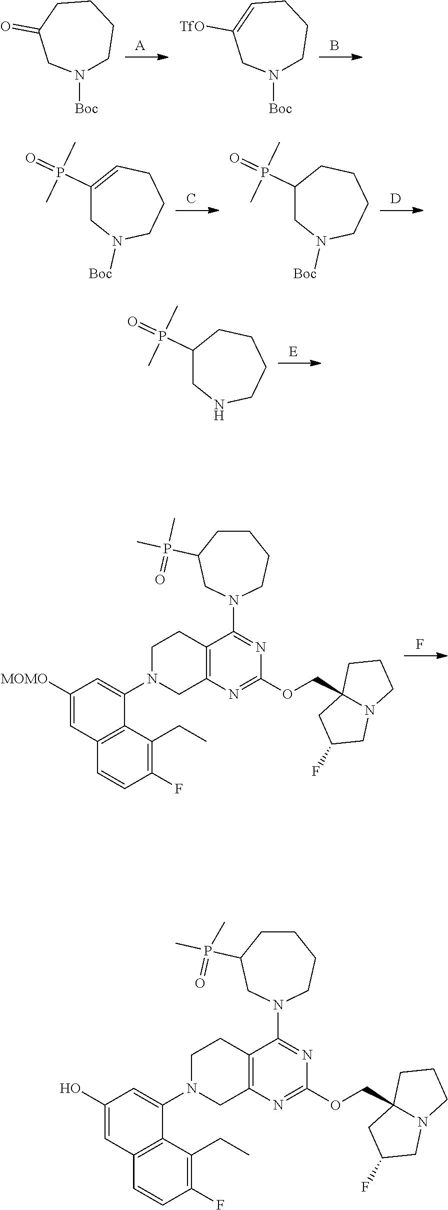

Step A. tert-butyl 6-Ui(trifluoromethyl)sulfonyl)oxy)-2.3.4.7-tetrahydro-1H-azepine-1-carboxylate: To a solution of tert-butyl 3-oxoazepane-1-carboxylate (5.00 g, 1.0 equiv) in THE (15 mL) was slowly added LiHMDS (1.0 M, 28.1 mL, 1.2 equiv) at −78° C., and the reaction was stirred at −78° C. for 1 hour. A solution of 1,1,1-trifluoro-N-phenyl-N-(trifluoromethylsulfonyl)methanesulfonamide (10.9 g, 1.3 equiv) in THF (15 mL) was added dropwise at −78° C. and the reaction was stirred at −78° C. for 15 minutes and then at 25° C. for 12 hours. The mixture was concentrated and purified with column chromatography [SiO2, petroleum ether/ethyl acetate=I/O to 10/1] to afford the title compound (6.6 g, 78% yield) as a yellow oil. 1H NMR (400 MHz, CHLOROFORM-d) δ=7.14-6.79 (m, 1H), 3.70 (br t, J=5.6 Hz, 2H), 2.65-2.55 (m, 2H), 1.92-1.75 (m, 4H), 1.50 (s, 9H); LCMS (ESI, M-56+1): m/z=290.1.

Step B. tert-butyl 6-(dimethylphosphoryl)-2,3,4,7-tetrahydro-1H-azepine-1-carboxylate: To a solution of tert-butyl 6-(((trifluoromethyl)sulfonyl)oxy)-2,3,4,7-tetrahydro-1H-azepine-1-carboxylate (500 mg, 1.0 equiv), methylphosphonoylmethane (136 mg, 1.2 equiv) and TEA (220 mg, 1.5 equiv) in ACN (5 mL) was added Pd(PPh3)4 (50.2 mg, 0.03 equiv). The reaction was degassed and purged with N2 3 times and stirred at 90° C. for 3 hours under N2 atmosphere. The mixture was filtered, concentrated, and purified with prep-HPLC [C18, 0.1% TFA condition] to afford the title compound (180 mg, 43% yield) as a yellow gum. 1H NMR (400 MHz, CHLOROFORM-d) S=7.37 (d, J=16.0 Hz, 1H), 3.80 (br s, 2H), 2.33 (br d, J=4.0 Hz, 2H), 1.88 (br s, 4H), 1.64 (d, J=12.8 Hz, 6H), 1.51 (s, 9H); LCMS (ESI, M+1): m/z=274.3.

Step C. tert-butyl 3-(dimethylphosphoryl)azepane-1-carboxylate: To a solution of tert-butyl 6-(dimethylphosphoryl)-2,3,4,7-tetrahydro-1H-azepine-1-carboxylate (180 mg, 1.0 equiv) in MeOH (2 mL) was added Pd/C (10%, 20 mg) under N2 atmosphere. The suspension was degassed and purged with H2 3 times. The reaction was stirred under H2 (15 Psi) at 25° C. for 12 hours. The mixture was filtered and concentrated to afford the title compound (160 mg) as a yellow oil. 1H NMR (400 MHz, CHLOROFORM-d) δ=4.02-3.85 (m, 1H), 3.33-3.24 (m, 1H), 3.21-3.06 (m, 1H), 2.58-2.28 (m, 4H), 2.19-1.89 (m, 4H), 1.64-1.57 (m, 6H), 1.49-1.44 (m, 9H).

Step D. azepan-3-yldimethylphosphine oxide: To a solution of tert-butyl 3-(dimethylphosphoryl) azepane-1-carboxylate (160 mg, 1.0 equiv) in dichloromethane (1.0 mL) was added HCl•dioxane (4 M, 1 mL, 6.9 equiv). The mixture was stirred at 25° C. for 2 hours. The reaction was concentrated under reduced pressure to afford the title compound (120 mg, HCl salt) as a yellow oil.

The last two steps were performed according to Example 248 except that K3P04 instead of N,N-diethylpropan-2-amine was used in step E. The title compound was obtained as yellow solid (FA salt). 1H NMR (400 MHz, METHANOL-d4) δ=8.54 (s, 1H), 7.50 (dd, J=6.0, 8.4 Hz, 1H), 7.13 (t, J=9.2 Hz, 1H), 7.02-6.91 (m, 2H), 5.41-5.16 (m, 1H), 4.60-4.45 (m, 1H), 4.25-3.91 (m, 4H), 3.76-3.57 (m, 2H), 3.53-3.32 (m, 4H), 3.28-3.09 (m, 4H), 3.07-2.98 (m, 1H), 2.80-2.69 (m, 1H), 2.33 (br dd, J=3.6, 14.8 Hz, 1H), 2.28-2.09 (m, 2H), 2.08-1.89 (m, 6H), 1.89-1.64 (m, 3H), 1.64-1.56 (m, 6H), 1.48-1.26 (m, 1H), 1.09 (t, J=7.2 Hz, 3H); LCMS (ESI, M+1): m/z=654.3.

### Example 352

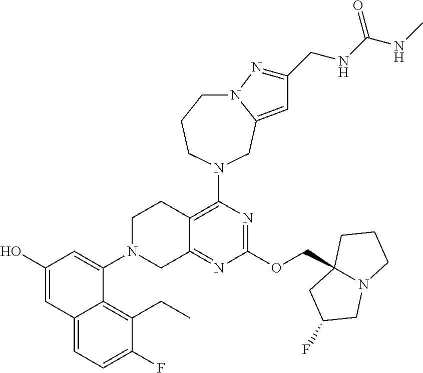 [SMILES: CCc1c(F)ccc2cc(O)cc(N3CCc4c(nc(OC[C@@]56CCCN5C[C@H](F)C6)nc4N4CCCn5nc(CNC(=O)NC)cc5C4)C3)c12]

### 1-((5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methyl)-3-methylurea

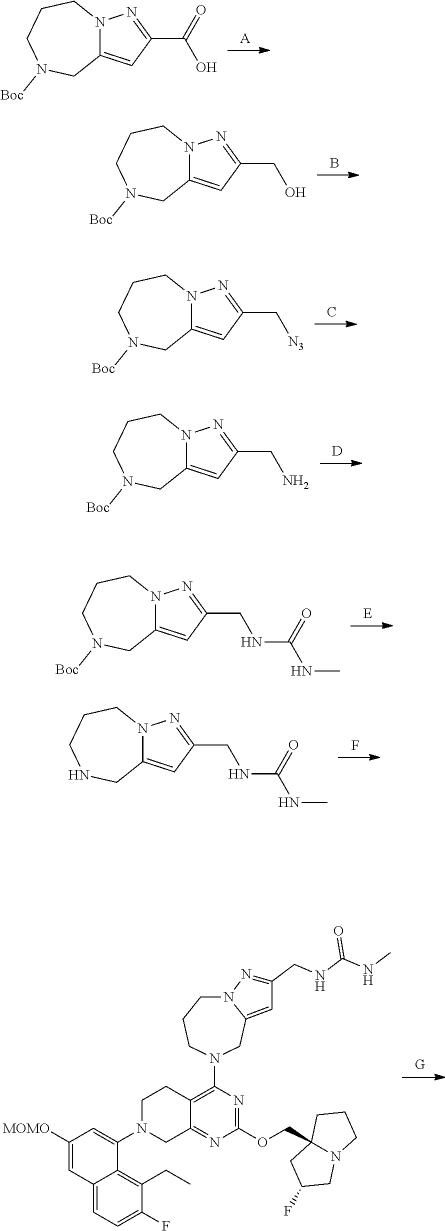

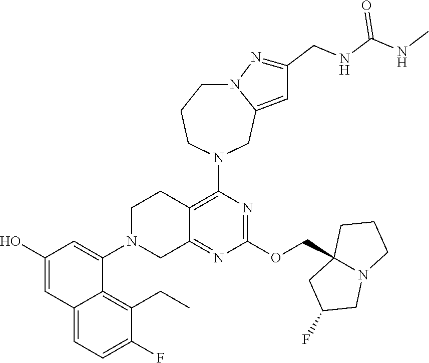 [SMILES: CCc1c(F)ccc2cc(O)cc(N3CCc4c(nc(OC[C@@]56CCCN5C[C@H](F)C6)nc4N4CCCn5nc(CNC(=O)NC)cc5C4)C3)c12]

Step A. tert-butyl 2-(hydroxymethyl)-7,8-dihydro-4H-pyrazolo[1,5-a][1,4]diazepine-5(6H)-carboxylate: To a solution of 5-(tert-butoxycarbonyl)-5,6,7,8-tetrahydro-4H-pyrazolo[1,5-a][1,4]diazepine-2-carboxylic acid (5.00 g, 1.0 equiv) in THE (50 mL) was added BH3Me2S (10 M, 18 mL, 10 equiv) at 0° C. The reaction was stirred at 60° C. for 1 hour. MeOH was added dropwise at 0° C. and then the reaction was stirred at 60° C. for 0.5 hours. The mixture was concentrated to afford the title compound (5.00 g, crude) as white solid; LCMS (ESI, M+1): m/z=268.2.

Step B. tert-butyl 2-(azidomethyl)-7,8-dihydro-4H-pyrazolo[1,5-a][1,4]diazepine-5(6H)-carboxylate: To a solution of tert-butyl 2-(hydroxymethyl)-7,8-dihydro-4H-pyrazolo[1,5-a][1,4]diazepine-5(6H)-carboxylate (5.00 g, 1.0 equiv) in DCM (50 mL) were added DPPA (7.72 g, 1.5 equiv) and DBU (4.27 g, 1.5 equiv). The reaction was stirred at 40° C. for 12 hours. The reaction was concentrated and purified by reversed phase HPLC (0.1% FA condition) to afford the title compound (2.78 g, 42% yield) as yellow oil, LCMS (ESI, M+1): m/z=293.0.

Step C. tert-butyl 2-(aminomethyl)-7,8-dihydro-4H-pyrazolo[1,5-alr1,4]diazepine-5(6H)-carboxylate: To a solution of tert-butyl 2-(azidomethyl)-7,8-dihydro-4H-pyrazolo[1,5-a][1,4]diazepine-5(6H)-carboxylate (2.00 g, 1.0 equiv) in THF (20 mL) was added PPh3 (3.59 g, 2.0 equiv). The reaction was stirred at 60° C. for 2 hours. The mixture was concentrated and purified by prep-HPLC (column: YMC Triart C18 250×50 mm×7 μm; mobile phase: [water (NH₃H20) ACN]; B %: 16%-36%, 17 min) and lyophilized to afford the title compound (0.8 g, 43% yield) as black oil; LCMS (ESI, M+1): m/z=267.0.

Step D. tert-butyl 2-((3-methylureido)methyl)-7,8-dihydro-4H-pyrazolo[1,5-a][1,4]diazepine-5(6H)-carboxylate: To a solution of tert-butyl 2-(aminomethyl)-7,8-dihydro-4H-pyrazolo[1,5-a][1,4]diazepine-5(6H)-carboxylate (68.0 mg, 1.5 equiv) in DCM (1 mL) was added TEA (148 mg, 3.0 equiv). The reaction was stirred at 20° C. for 0.5 hours. The reaction was concentrated and purified by reversed phase HPLC (0.1% FA condition) to afford the title compound (140 mg, 83% yield) as yellow solid; LCMS (ESI, M+1): m/z=324.2.

Step E. 1-methyl-3-((5,6,7,8-tetrahydro-4H-pyrazolo[1,5-a][1,4]diazepin-2-yl)methyl)urea: To a solution of tert-butyl 2-((3-methylureido)methyl)-7,8-dihydro-4H-pyrazolo[1,5-a][1,4]diazepine-5(6H)-carboxylate (130 mg, 1.0 equiv) in MeCN (0.5 mL) was added HCl•dioxane (4 M, 1 mL, 10 equiv). The reaction was stirred at 20° C. for 0.5 hours. The reaction was concentrated under reduced pressure to afford the title compound (90 mg, crude) as yellow solid.

The last two steps were performed according to Example 248. The title compound was obtained as off-white solid (FA salt). 1H NMR (400 MHz, METHANOL-d4) δ=7.52 (dd, J=6.0, 9.2 Hz, 1H), 7.15 (t, J=9.2 Hz, 1H), 6.99-6.94 (m, 2H), 6.16 (s, 1H), 5.48-5.30 (m, 1H), 5.01-4.92 (m, 1H), 4.82-4.75 (m, 1H), 4.50-4.37 (m, 2H), 4.31-4.15 (m, 5H), 4.07 (d, J=17.6 Hz, 1H), 4.02-3.92 (m, 1H), 3.69 (br d, J=17.6 Hz, 1H), 3.59-3.44 (m, 4H), 3.43-3.35 (m, 2H), 3.26-3.15 (m, 3H), 2.71 (s, 4H), 2.50-2.36 (m, 1H), 2.35-2.17 (m, 3H), 2.16-1.95 (m, 4H), 1.15-1.09 (m, 3H); LCMS (ESI, M+1): m/z=702.3.

### Example 353

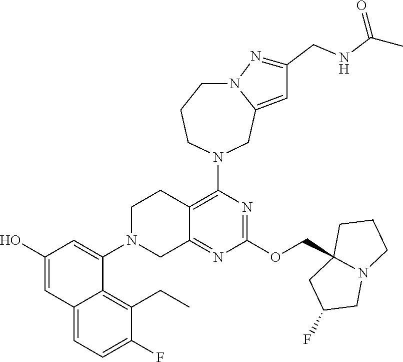 [SMILES: CCc1c(F)ccc2cc(O)cc(N3CCc4c(nc(OC[C@@]56CCCN5C[C@H](F)C6)nc4N4CCCn5nc(CNC(C)=O)cc5C4)C3)c12]

### N-((5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methyl)acetamide

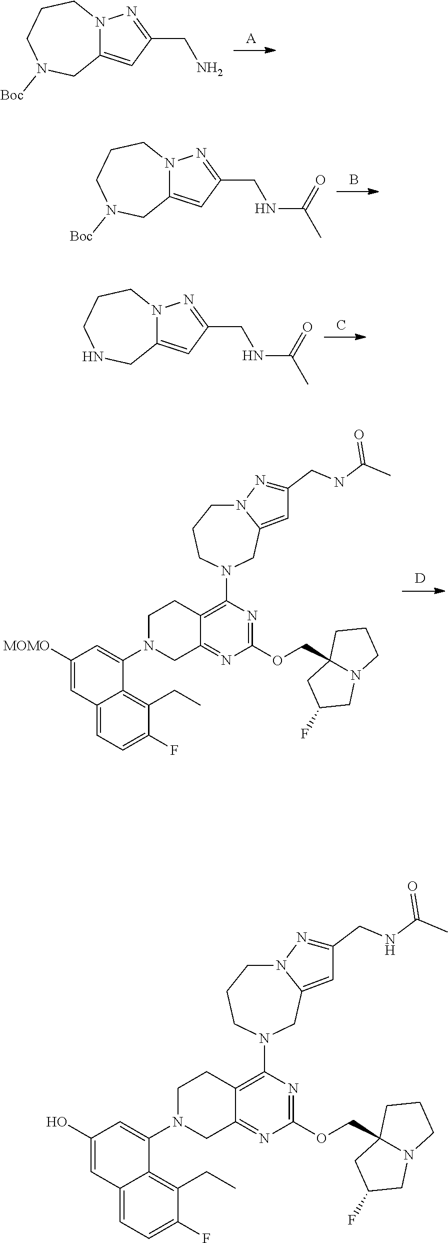

Step A. tert-butyl 2-(acetamidomethyl)-7,8-dihydro-4H-pyrazolo[1,5-a][1,4]diazepine-5(6H)-carboxylate: To a mixture of tert-butyl 2-(aminomethyl)-7,8-dihydro-4H-pyrazolo[1,5-a][1,4]diazepine-5(6H)-carboxylate (120 mg, 1.0 equiv) and acetic anhydride (69.0 mg, 1.5 equiv) in DCM (1.5 mL) was added TEA (137 mg, 3.0 equiv). The reaction was stirred at 25° C. for 2 hours. The mixture was concentrated and purified by reversed phase flash chromatography [water (FA, 0.1%)/acetonitrile] to afford the title compound (120 mg, 86% yield) as a yellow oil. LCMS (ESI, M+1): m/z=309.1.

Step B. N-((5,6,7,8-tetrahydro-4H-pyrazolo[1,5-a][1,4]diazepin-2-yl)methyl)acetamide: To a mixture of tert-butyl 2-(acetamidomethyl)-7,8-dihydro-4H-pyrazolo[1,5-a][1,4]diazepine-5(6H)-carboxylate (100 mg, 1.0 equiv) in MeCN (1.0 mL) was added HCl•dioxane (4 M, 0.8 mL, 10 equiv). The reaction was stirred at 25° C. for 1 hour. The mixture was concentrated to afford the title compound (60 mg, crude, HCl) as yellow solid and used into next step without further purification.

The last two steps were performed according to Example 248. The title compound was obtainedas off-white solid (FA salt). 1H NMR (400 MHz, METHANOL-d4) δ=7.58-7.55 (m, 1H), 7.15 (t, J=9.6 Hz, 1H), 6.97 (br d, J=4.4 Hz, 2H), 6.17 (s, 1H), 5.51-5.26 (m, 1H), 5.07-4.92 (m, 1H), 4.79-4.76 (m, 1H), 4.44 (br d, J=4.4 Hz, 2H), 4.24-4.14 (m, 3H), 4.38-4.13 (m, 3H), 4.11-4.01 (m, 1H), 4.00-3.90 (m, 1H), 3.69 (br d, J=17.6 Hz, 1H), 3.55-3.46 (m, 2H), 3.45-3.36 (m, 3H), 3.24-3.15 (m, 3H), 2.73 (br d, J=12.8 Hz, 1H), 2.47-2.33 (m, 1H), 2.33-2.16 (m, 3H), 2.16-1.98 (m, 4H), 1.97 (s, 3H), 1.18-1.05 (m, 3H). LCMS (ESI, M+1): m/z=687.5.

### Example 354

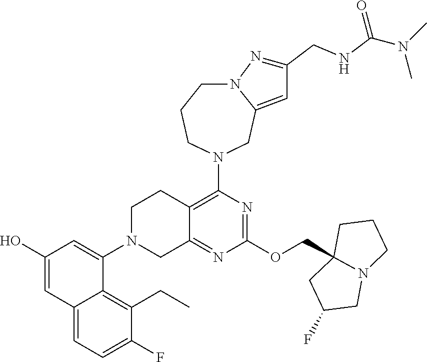 [SMILES: CCc1c(F)ccc2cc(O)cc(N3CCc4c(nc(OC[C@@]56CCCN5C[C@H](F)C6)nc4N4CCCn5nc(CNC(=O)N(C)C)cc5C4)C3)c12]

### 3-((5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methyl)-1,1-dimethylurea

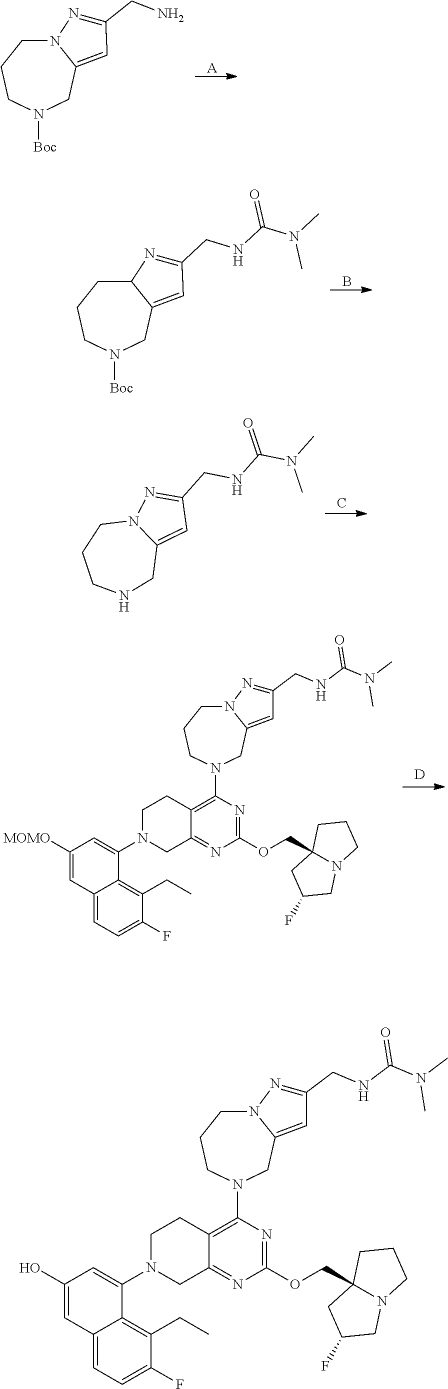

Step A. tert-butyl 2-((3,3-dimethylureido)methyl)-7,8-dihydro-4H-pyrazolo[1,5-a][1,4]diazepine-5(6H)-carboxylate: To a mixture of tert-butyl 2-(aminomethyl)-7,8-dihydro-4H-pyrazolo[1,5-a][1,4]diazepine-5(6H)-carboxylate (130 mg, 1.0 equiv) and TEA (148 mg, 3.0 equiv) in DCM (2 mL) was added dimethylcarbamic chloride (79.0 mg, 1.5 equiv). The reaction was stirred at 25° C. for 1 hour. The mixture was concentrated and purified by reversed phase flash chromatography [C18, 0.1% formic acid condition] to afford the title compound (150 mg, 90% yield) as yellow solid. LCMS (ESI, M+1): m/z=338.2.

Step B. 1,1-dimethyl-3-((5,6,7,8-tetrahydro-4H-pyrazolo[1,5-a][1,4]diazepin-2-yl)methyl)urea: To a mixture of tert-butyl 2-((3,3-dimethylureido)methyl)-7,8-dihydro-4H-pyrazolo[1,5-a][1,4]diazepine-5(6H)-carboxylate (140 mg, 1.0 equiv) in MeCN (0.5 mL) was added HCl•dioxane (4 M, 0.5 mL). The reaction was stirred at 25° C. for 0.5 hours. The mixture was filtered and concentrated to afford the title compound (150 mg, crude, HCl) as white oil.

The last two steps were performed according to Example 248. The title compound was obtained as off-white solid. 1H NMR (400 MHz, METHANOL-d4) δ=7.52 (dd, J=5.6, 8.8 Hz, 1H), 7.15 (t, J=9.2 Hz, 1H), 6.97 (s, 2H), 6.13 (s, 1H), 5.41-5.16 (m, 1H), 5.02 (br d, J=16.0 Hz, 1H), 4.70 (br d, J=3.2 Hz, 1H), 4.44 (br d, J=5.6 Hz, 2H), 4.36-4.30 (m, 1H), 4.26-4.20 (m, 2H), 4.13-4.04 (m, 2H), 4.04-3.99 (m, 1H), 3.93-3.82 (m, 1H), 3.67 (br d, J=17.6 Hz, 1H), 3.55-3.46 (m, 1H), 3.39 (br d, J=5.6 Hz, 2H), 3.23-3.17 (m, 4H), 3.13 (br s, 1H), 3.02-2.86 (m, 7H), 2.76-2.68 (m, 1H), 2.35-2.18 (m, 2H), 2.14 (br d, J=4.8 Hz, 1H), 2.10-2.02 (m, 2H), 2.00-1.79 (m, 3H), 1.13 (br t, J=7.2 Hz, 3H); LCMS (ESI, M+1): m/z=716.6.

### Example 355

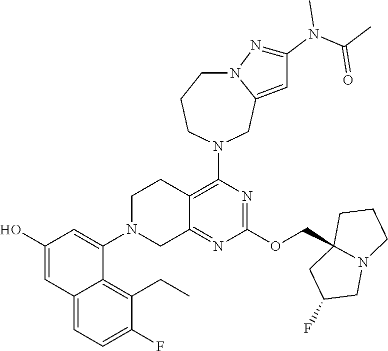 [SMILES: CCc1c(F)ccc2cc(O)cc(N3CCc4c(nc(OC[C@@]56CCCN5C[C@H](F)C6)nc4N4CCCn5nc(N(C)C(C)=O)cc5C4)C3)c12]

### N-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1_4]diazepin-2-yl)-N-methylacetamide

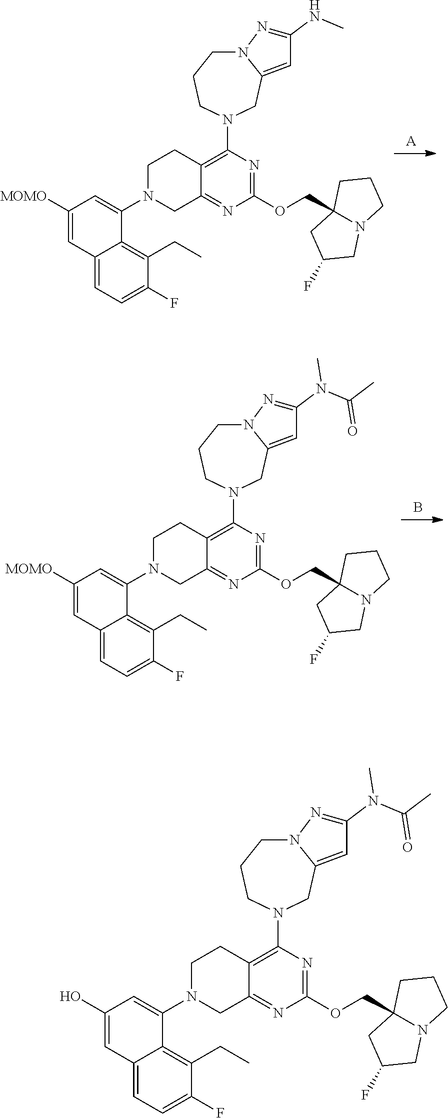

Step A. N-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-methylacetamide: To a mixture of 5-(7-(8-ethyl-7-fluoro-3-(methoxymethoxy)naphthalen-1-yl)-2-(((2R,7aS)-2-fluorotetrahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepin-2-amine (50.0 mg, 1.0 equiv) in THF (0.5 mL) was added NaH (4.35 mg, 60% purity, 1.5 equiv) at 0° C. The reaction was stirred at 20° C. for 30 minutes. Ac20 (14.8 mg, 2.0 equiv) in THF (0.5 mL) was added at 0° C. The reaction was stirred at 20° C. for 1 hour. The mixture was diluted with saturated NaHCO₃ aqueous solution (5 mL) and extracted with EtOAc (3×5 mL). The combined organic layers were washed with brine (10 mL), dried over anhydrous sodium sulfate and concentrated to afford the title compound (50.0 mg, 94% yield) as yellow solid; LCMS (ESI, M+1): m/z=731.4.

Step B. N-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-methylacetamide: To a mixture ofN-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-methylacetamide (50.0 mg, 1.0 equiv) in ACN (0.5 mL) was added HCl-MeOH (4 M, 0.5 mL) at 0° C. The reaction was stirred at 20° C. for 30 minutes. The mixture was concentrated and purified by prep-HPLC [Welch Xtimate C18 150 x 25 mm×5 μm; A: water (NH₃H20), B: ACN; B %: 38%-68% over 8 min]followed by prep-HPLC [Phenomenex luna C18 150×25 mm×10 μm; A: water (FA), B: ACN; B %: 21%-51% over 10 min] to afford the title compound (4.61 mg) as off-white solid (FA salt). 1H NMR (400 MHz, methanol-d4) δ=7.57-7.49 (m, 1H), 7.21-7.11 (m, 1H), 7.04-6.91 (m, 2H), 6.28-6.19 (m, 1H), 5.45-5.24 (m, 1H), 5.09-4.94 (m, 1H), 4.83-4.57 (m, 1H), 4.53-4.41 (m, 2H), 4.27-4.17 (m, 2H), 4.16-3.98 (m, 3H), 3.76-3.64 (m, 1H), 3.59-3.52 (m, 1H), 3.51-3.36 (m, 5H), 3.25-3.17 (m, 4H), 3.16-3.14 (m, 1H), 2.82-2.72 (m, 1H), 2.41-2.19 (m, 4H), 2.17-1.83 (m, 8H), 1.16-1.09 (m, 3H); 19F NMR (400 MHz, methanol-d4) δ=−122.962, -173.748; LCMS (ESI, M+1): m/z=687.4.

### Example 356

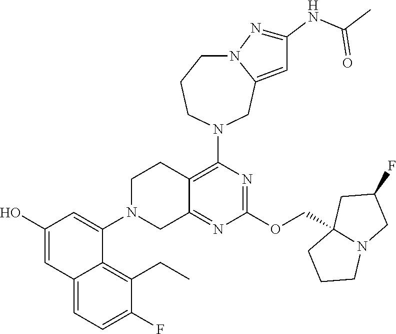 [SMILES: CCc1c(F)ccc2cc(O)cc(N3CCc4c(nc(OC[C@@]56CCCN5C[C@H](F)C6)nc4N4CCCn5nc(NC(C)=O)cc5C4)C3)c12]

### N-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)acetamide

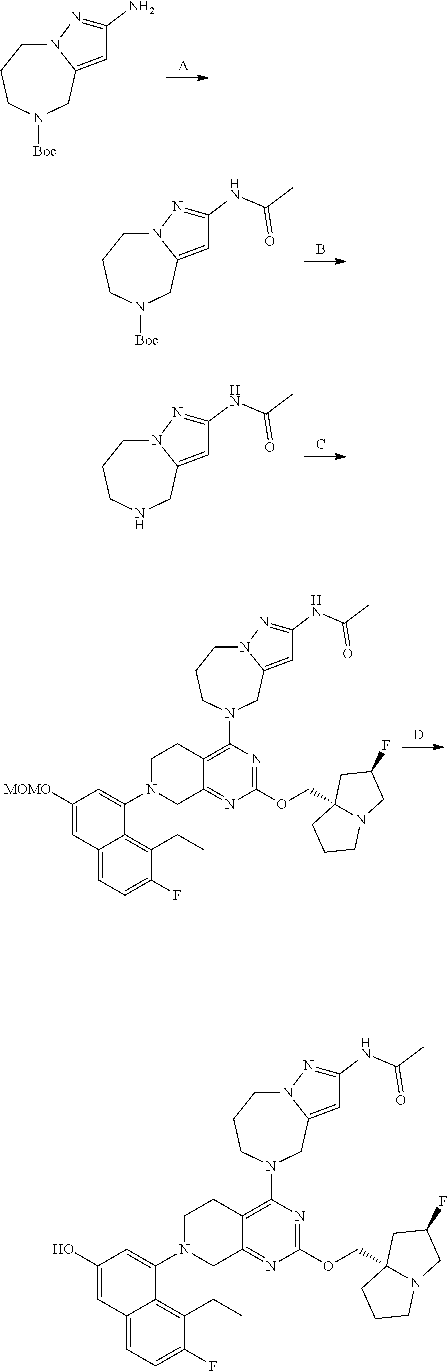

Step A. tert-butyl 2-acetamido-7,8-dihydro-4H-pyrazolo[1,5-a][1,4]diazepine-5(6H)-carboxylate: To a mixture of tert-butyl 2-amino-4,6,7,8-tetrahydropyrazolo[1,5-a][1,4]diazepine-5-carboxylate (100 mg, 1.0 equiv) and N,N-diethylpropan-2-amine (154 mg, 3.0 equiv) in DCM (1 mL) was added acetyl chloride (46.7 mg, 1.5 equiv) at 0° C. The reaction was stirred at 20° C. for 1 hour. The mixture was concentrated and purified by column chromatography (SiO2, DCM/MeOH=10/1) to afford the title compound (75.0 mg, 59% yield) as yellow solid; LCMS (ESI, M-55): m/z=239.0.

Step B. N-(5,6,7,8-tetrahydro-4H-pyrazolo[1,5-a][1,4]diazepin-2-yl)acetamide To a mixture of tert-butyl 2-acetamido-4,6,7,8-tetrahydropyrazolo[1,5-a][1,4]diazepine-5-carboxylate (150 mg, 1.0 equiv) in MeCN (1 mL) was added HCl•dioxane (4 M, 1 mL, 7.8 equiv). The reaction was stirred at 25° C. for 1 hour. The mixture was concentrated to afford the title compound (100 mg, crude) as yellow solid.

The last two steps were performed according to Example 248. The title compound was obtained as yellow solid. 1H NMR (400 MHz, METHANOL-d4) δ=7.59-7.41 (m, 1H), 7.23-7.05 (m, 1H), 7.03-6.91 (m, 2H), 6.62 (d, J=5.6 Hz, 1H), 5.68 (s, 1H), 4.98-4.89 (m, 1H), 4.78 (br d, J=3.2 Hz, 1H), 4.60-4.26 (m, 4H), 4.23-3.99 (m, 3H), 3.98-3.62 (m, 4H), 3.61-3.47 (m, 1H), 3.42-3.34 (m, 2H), 3.22 (br d, J=1.6 Hz, 4H), 2.83-2.38 (m, 4H), 2.35-2.20 (m, 3H), 2.10 (s, 3H), 2.04-1.93 (m, 1H), 1.10 (t, J=7.2 Hz, 3H), LCMS (ESI, M+1): m/z=673.4.

### Example 357

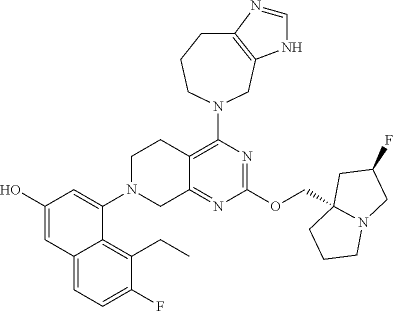 [SMILES: CCc1c(F)ccc2cc(O)cc(N3CCc4c(nc(OC[C@@]56CCCN5C[C@H](F)C6)nc4N4CCCc5nc[nH]c5C4)C3)c12]

### 4-(4-(7,8-dihydroimidazo[4,5-c]azepin-5(3H,4H,6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

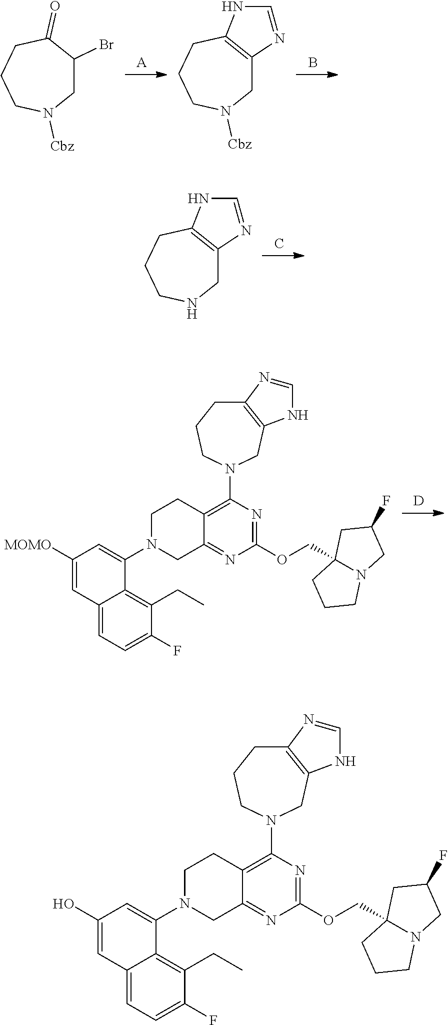

Step A. benzyl 4,6,7,8-tetrahydroimidazo[4,5-c]azepine-5(1H)-carboxylate: A mixture of benzyl 3-bromo-4-oxoazepane-1-carboxylate (1.00 g, 1.0 equiv) and formimidamide (958 mg, 3.0 equiv) in t-BuOH (10 mL) was stirred at 70° C. for 12 hours. The mixture was concentrated and purified by reversed phase flash chromatography [water (FA, 0.1%)/acetonitrile] to afford the title compound (240 mg, 28% yield) as a yellow oil. LCMS (ESI, M+1): m/z=271.8.

Step B. 1,4,5,6,7,8-hexahydroimidazo[4,5-c]azepine: To a mixture of benzyl 4,6,7,8-tetrahydroimidazo[4,5-c]azepine-5(1H)-carboxylate (100 mg, 1.0 equiv) in MeOH (2.0 mL) was added Pd/C (400 mg, 10%). The mixture was degassed and purged with N2 for 3 mins. The reaction was stirred at 25° C. for 1 hour under H2 at 15 psi. The mixture was filtered and concentrated to afford the title compound (80.0 mg, crude) as white oil and used into next step without further purification.

The last two steps were performed according to Example 248. The title compound was obtained as off-white solid (FA salt). 1H NMR (400 MHz, METHANOL-d4) δ=7.59 (d, J=4.0 Hz, 1H), 7.51 (dd, J=5.6, 8.8 Hz, 1H), 7.14 (t, J=9.6 Hz, 1H), 6.97 (s, 2H), 5.58-5.36 (m, 1H), 5.05 (s, 1H), 4.64 (br dd, J=6.4, 15.6 Hz, 1H), 4.41-4.34 (m, 1H), 4.34-4.26 (m, 2H), 4.03 (br d, J=17.6 Hz, 1H), 3.95-3.84 (m, 1H), 3.83-3.70 (m, 2H), 3.70-3.58 (m, 2H), 3.56-3.47 (m, 1H), 3.39-3.34 (m, 2H), 3.24-3.16 (m, 2H), 2.95-2.78 (m, 2H), 2.75 (br d, J=12.8 Hz, 1H), 2.64-2.48 (m, 1H), 2.48-2.36 (m, 2H), 2.35-2.15 (m, 3H), 2.14-1.99 (m, 2H), 1.93-1.78 (m, 1H), 1.10 (br t, J=7.2 Hz, 3H). LCMS (ESI, M+1): m/z=616.4

### Example 358

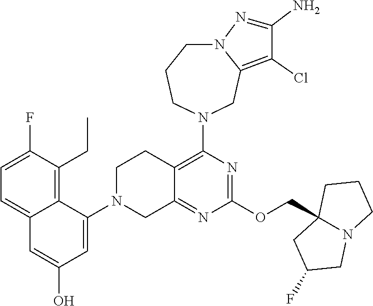 [SMILES: CCc1c(F)ccc2cc(O)cc(N3CCc4c(nc(OC[C@@]56CCCN5C[C@H](F)C6)nc4N4CCCn5nc(N)c(Cl)c5C4)C3)c12]

### 4-(4-(2-amino-3-chloro-7,8-dihydro-4H-pyrazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

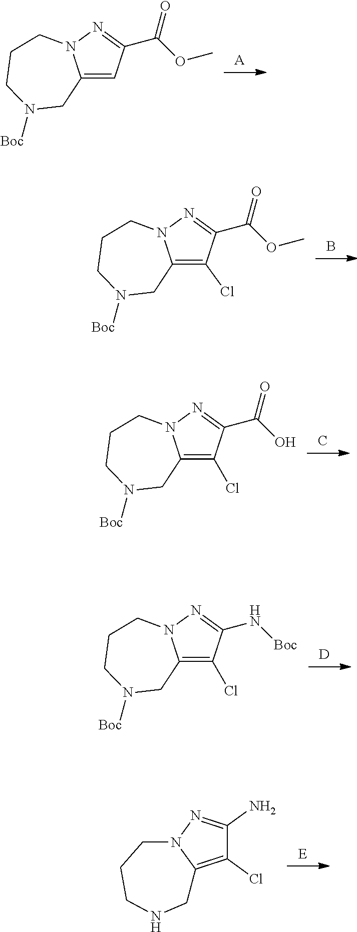

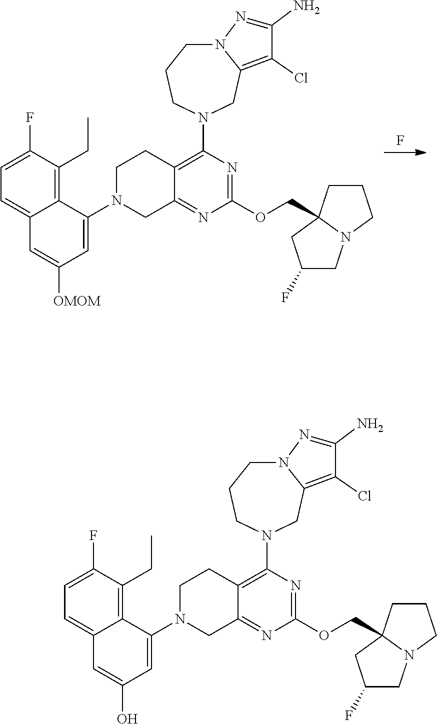

Step A. 5-tert-butyl 2-methyl 3-chloro-7,8-dihydro-4H-pyrazolo[1,5-a][1,4]diazepine-2,5(6H)-dicarboxylate: To a solution of 05-tert-butyl 02-methyl 4,6,7,8-tetrahydropyrazolo[1,5-a][1,4]diazepine-2,5-dicarboxylate (48.0 g, 1.0 equiv) in AcOH (500 mL) was added NCS (43.4 g, 2.0 equiv). The reaction was stirred at 80° C. for 2 hours. The mixture was concentrated under vacuum. Then the mixture was neutralized with saturated NaHCO₃ solution (1 L) and extracted with EtOAc (500 mL×2). The combined organic phase was washed with brine (300 mL), dried over anhydrous sodium sulfate, and concentrated to afford the title compound (65.6 g, crude) as yellow oil; 1H NMR (400 MHz, CHLOROFORM-d) δ=4.52 (br s, 2H), 4.51-4.47 (m, 2H), 3.94 (s, 3H), 3.82-3.69 (m, 2H), 1.98 (br s, 2H), 1.44 (s, 9H),

Step B. 5-(tert-butoxycarbonyl)-3-chloro-5,6,7,8-tetrahydro-4H-pyrazolo[1,5-a][1,4]diazepine-2-carboxylic acid: To a solution of 05-tert-butyl 02-methyl 3-chloro-4,6,7,8-tetrahydropyrazolo[1,5-a][1,4]diazepine-2,5-dicarboxylate (65.6 g, 1.0 equiv) in MeOH (450 mL) and H₂O (150 mL) was added LiOH (23.8 g, 5.0 equiv). The reaction was stirred at 20° C. for 16 hours. The mixture was concentrated under vacuum to remove MeOH. Then the mixture was diluted with H₂O (1 L) and extracted with ethyl acetate (1L x 2). Then the aqueous phase was neutralized with 1M HCl solution (50 mL) and extracted with EtOAc (500 mL). The combined organic layers were washed with brine (500 mL), dried over anhydrous sodium sulfate, and concentrated to afford the title compound (36.0 g, crude) as yellow solid; 1H NMR (400 MHz, CHLOROFORM-d) δ=4.52 (br dd, J=5.6, 10.9 Hz, 4H), 3.82-3.70 (m, 2H), 2.06-1.93 (m, 2H), 1.54-1.39 (m, 9H); LCMS (ESI, M+1): m/z=316.1.

Step C. tert-butyl 2-((tert-butoxycarbonyl)amino)-3-chloro-7,8-dihydro-4H-pyrazolo[1,5-a][1,4]diazepine-5(6H)-carboxylate: To a mixture of 5-tert-butoxycarbonyl-3-chloro-4,6,7,8-tetrahydropyrazolo[1,5-a][1,4]diazepine-2-carboxylic acid (26.0 g, 1.0 equiv) in toluene (260 mL) were added 4 A molecular sieve (1.00 g), TEA (34.9 mL, 3.0 equiv), DPPA (34.0 g, 1.5 equiv) and t-BuOH (183 g, 30 equiv) and the reaction was stirred at 110° C. for 5 hours under nitrogen. The reaction was diluted with ethyl acetate (1 L) and water (2 L) and extracted with ethyl acetate (500 mL). The combined organic layers were washed with brine (500 mL), dried over anhydrous sodium sulfate, filtered, and concentrated to give a residue. The residue was purified by column chromatography (SiO2, petroleum ether/ethyl acetate 30/1 to 1/1) to afford the title compound (18.0 g, 54% yield) as yellow solid; 1H NMR (400 MHz, CHLOROFORM-d) δ=6.29 (br s, 1H), 4.44 (br s, 2H), 4.38-4.33 (m, 2H), 3.71 (br s, 2H), 1.94 (br s, 2H), 1.51 (s, 9H), 1.44 (s, 9H); LCMS (ESI, M+1): m/z=387.2.

Step D. 3-chloro-5,6,7,8-tetrahydro-4H-pyrazolo[1,5-a][1,4]diazepin-2-amine: A mixture of tert-butyl 2-(tert-butoxycarbonylamino)-3-chloro-4,6,7,8-tetrahydropyrazolo[1,5-a][1,4]diazepine-5-carboxylate (12.0 g, 1.0 equiv) in HCl•MeOH (100 mL) was stirred at 20° C. for 3 hours. The mixture was concentrated under vacuum to afford the title compound (6.9 g, crude, HCl) as yellow solid; 1H NMR (400 MHz, METHANOL-d4) δ=4.59-4.48 (m, 4H), 3.66-3.57 (m, 2H), 2.24-2.14 (m, 2H); LCMS (ESI, M+1): m/z=187.0.

The last two steps were performed according to Example 248. The title compound was obtained as yellow oil (FA salt). 1 1H NMR (400 MHz, DMSO-d6) δ=9.70 (br s, 1H), 7.59 (br s, 1H), 7.24 (br t, J=8.4 Hz, 1H), 6.98 (br s, 2H), 5.36-5.12 (m, 1H), 4.92-4.79 (m, 1H), 4.71 (br s, 2H), 4.56 (br d, J=16.4 Hz, 1H), 4.24-4.07 (m, 2H), 3.73 (br s, 4H), 3.77-3.67 (m, 1H), 3.60 (br d, J=17.2 Hz, 1H), 3.38 (br s, 1H), 3.22 (br d, J=8.6 Hz, 1H), 3.14-2.91 (m, 4H), 2.79 (br d, J=5.6 Hz, 1H), 2.65-2.55 (m, 1H), 2.24-2.10 (m, 1H), 2.05 (br d, J=10.4 Hz, 1H), 2.02-1.86 (m, 3H), 1.86-1.64 (m, 3H), 1.05 (br s, 3H); LCMS (ESI, M+1): m/z=665.3.

### Example 359

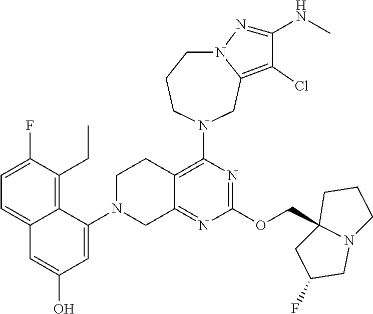 [SMILES: CCc1c(F)ccc2cc(O)cc(N3CCc4c(nc(OC[C@@]56CCCN5C[C@H](F)C6)nc4N4CCCn5nc(NC)c(Cl)c5C4)C3)c12]

### 4-(4-(3-chloro-2-(methylamino)-7,8-dihydro-4H-pyrazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

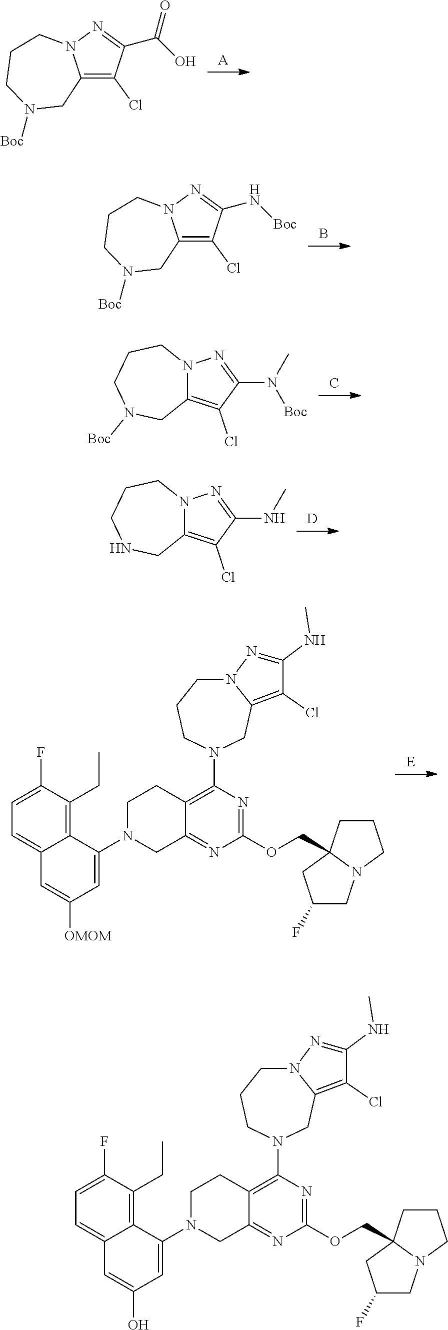

Step A. tert-butyl 2-((tert-butoxycarbonyl)amino)-3-chloro-7,8-dihydro-4H-pyrazolo[1,5-a][1,4]diazepine-5(6H)-carboxylate: To a mixture of 5-tert-butoxycarbonyl-3-chloro-4,6,7,8-tetrahydropyrazolo[1,5-a][1,4]diazepine-2-carboxylic acid (26.0 g, 1.0 equiv) in toluene (260 mL) were added 4 A molecular sieve (1.00 g), TEA (34.9 mL, 3.0 equiv), DPPA (34.0 g, 1.5 equiv) and t-BuOH (183 g, 30 equiv). The reaction was stirred at 110° C. for 5 hours under nitrogen. The reaction was diluted with EtOAc (1 L) and water (2 L), extracted with EtOAc (500 mL), and the combined organic layers were washed with brine (500 mL), dried over anhydrous sodium sulfate, filtered, and concentrated to give a residue. The residue was purified by column chromatography (SiO2, petroleum ether/ethyl acetate 30/1 to 1/1) to afford the title compound (18.0 g, 54% yield) as yellow solid; ¹H NMR (400 MHz, CHLOROFORM-d) δ=6.29 (br s, 1H), 4.44 (br s, 2H), 4.38-4.33 (m, 2H), 3.71 (br s, 2H), 1.94 (br s, 2H), 1.51 (s, 9H), 1.44 (s, 9H); LCMS (ESI, M+1): m/z=387.2.

Step B. tert-butyl 2-((tert-butoxycarbonyl)(methyl)amino)-3-chloro-7,8-dihydro-4H-pyrazolo[1,5-a][1,4]diazepine-5(6H)-carboxylate: To a solution of tert-butyl 2-(tert-butoxycarbonylamino)-3-chloro-4,6,7,8-tetrahydropyrazolo[1,5-a][1,4]diazepine-5-carboxylate (400 mg, 1.0 equiv) in THE (10 mL) was added NaH (82.7 mg, 60% purity, 2 0.0 equiv) in portions slowly at 0° C. for 1 hour. Then Mel (734 mg, 5.0 equiv) was added dropwised at 0° C. The mixture was stirred at 20° C. for 16 hours. The mixture was diluted with water (20 mL) at 0° C. for 1 minute and extracted with ethyl acetate (2×10 mL). The combined organic layers were dried over anhydrous sodium sulfate and concentrated to afford the title compound (420 mg, crude) as yellow oil; LCMS (ESI, M+1): m/z=401.1.

Step C. 3-chloro-N-methyl-5,6,7,8-tetrahydro-4H-pyrazolo[1,5-a][1,4]diazepin-2-amine: A mixture of tert-butyl 2-[tert-butoxycarbonyl(methyl)amino]-3-chloro-4,6,7,8-tetrahydropyrazolo[1,5-a][1,4]diazepine-5-carboxylate (420 mg, 1.0 equiv) in HCl•dioxane (5 mL) was stirred at 20° C. for 1 hour. The mixture was concentrated under vacuum to afford the title compound (210 mg, crude, HCl) as yellow solid.

The last two steps were performed according to Example 248. The title compound was obtained as white solid. 1H NMR (400 MHz, METHANOL-d4) δ=7.51 (dd, J=6.0, 9.0 Hz, 11H), 7.14 (t, J=9.2 Hz, 11H), 7.00-6.94 (m, 2H), 5.34-5.16 (m, 11H), 4.81 (dd, J=2.8, 16.4 Hz, 1H), 4.73-4.64 (m, 1H), 4.30-4.13 (m, 2H), 4.13-3.97 (m, 4H), 3.96-3.85 (m, 1H), 3.66 (d, J=18.0 Hz, 1H), 3.53-3.37 (m, 3H), 3.30-3.07 (m, 5H), 3.02-2.92 (m, 1H), 2.81 (s, 3H), 2.69 (br d, J=14.8 Hz, 1H), 2.37-2.17 (m, 2H), 2.17-2.01 (m, 3H), 2.00-1.79 (m, 3H), 1.10 (t, J=7.2 Hz, 3H); LCMS (ESI, M+1): m/z=679.3.

### Example 360

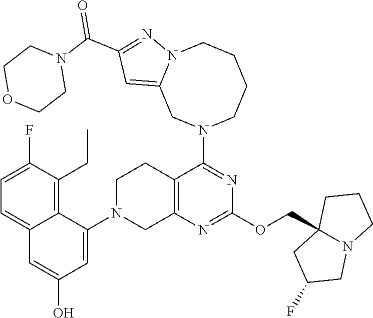 [SMILES: CCc1c(F)ccc2cc(O)cc(N3CCc4c(nc(OC[C@@]56CCCN5C[C@H](F)C6)nc4N4CCCCn5nc(C(=O)N6CCOCC6)cc5C4)C3)c12]

### (5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-4,5,6,7,8,9-hexahydropyrazolo[1,5-a][1,4]diazocin-2-yl)(morpholino)methanone

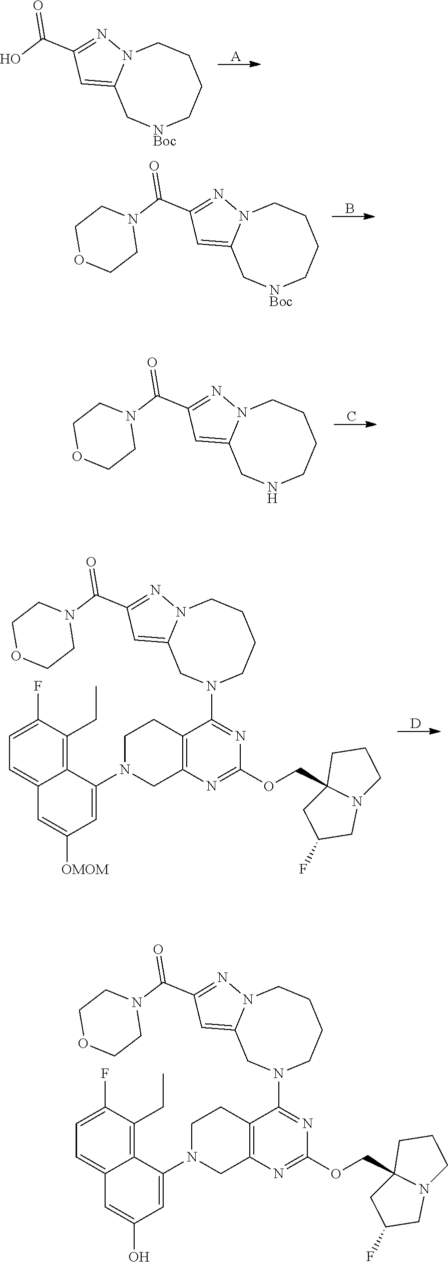

Synthesized according to Example 349. The title compound was obtained as light-yellow solid (FA salt). 1H NMR (400 MHz, CD3OD) 8=8.53 (s, 1H), 7.51 (dd, J=6.0, 8.8 Hz, 1H), 7.15 (t, J=9.2 Hz, 1H), 6.97 (br d, J=4.4 Hz, 2H), 6.59 (s, 1H), 5.44-5.20 (m, 1H), 5.08-4.93 (m, 2H), 4.45 (br t, J=5.2 Hz, 2H), 4.18-3.98 (m, 5H), 3.93-3.83 (mn, 1H), 3.80-3.65 (m, 8H), 3.49 (br d, J=7.2 Hz, 1H), 3.44-3.32 (m, 4H), 3.29 (br s, 1H), 3.25-3.15 (m, 2H), 3.12-3.03 (m, 1H), 2.70 (br d, J=13.6 Hz, 1H), 2.33-1.70 (m, 10H), 1.11 (t, J=7.2 Hz, 3H); LCMS (ESI, M+1). m/z=743.3.

### Example 361

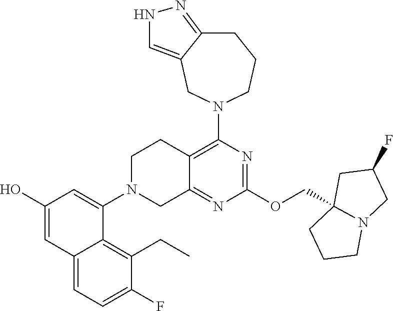 [SMILES: CCc1c(F)ccc2cc(O)cc(N3CCc4c(nc(OC[C@@]56CCCN5C[C@H](F)C6)nc4N4CCCc5n[nH]cc5C4)C3)c12]

### 4-(4-(7,8-dihydropyrazolo[4,3-c]azepin-5(2H,4H,6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

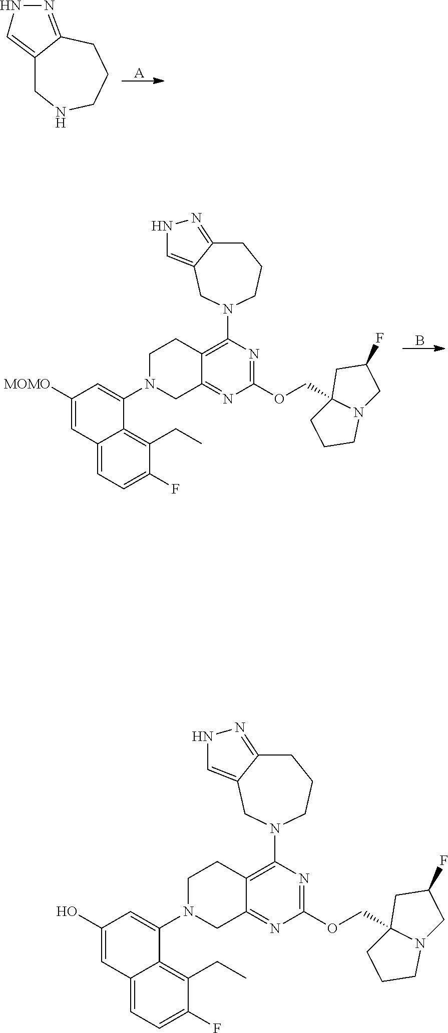

Synthesized according to Example 248. The title compound was obtained as white solid (FA salt). 1H NMR (400 MHz, METHANOL-d4) δ=7.56-7.46 (m, 2H), 7.14 (t, J=9.2 Hz, 1H), 6.96 (s, 2H), 5.45-5.28 (m, 1H), 4.77 (br s, 2H), 4.29-4.22 (m, 1H), 4.21-3.98 (m, 4H), 3.64 (br d, J=17.6 Hz, 1H), 3.55-3.39 (m, 4H), 3.37 (br d, J=7.2 Hz, 2H), 3.26-3.12 (m, 3H), 3.04-2.88 (m, 2H), 2.75 (br d, J=14.4 Hz, 1H), 2.42-1.84 (m, 8H), 1.15-1.01 (m, 3H). LCMS (ESI, M+1): m/z=616.4.

### Example 362

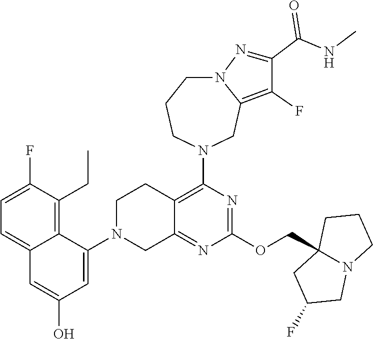 [SMILES: CCc1c(F)ccc2cc(O)cc(N3CCc4c(nc(OC[C@@]56CCCN5C[C@H](F)C6)nc4N4CCCn5nc(C(=O)NC)c(F)c5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-3-fluoro-N-methyl-5,6,7,8-tetrahydro-4H-pyrazolo[1,5-a][1,4]diazepine-2-carboxamide

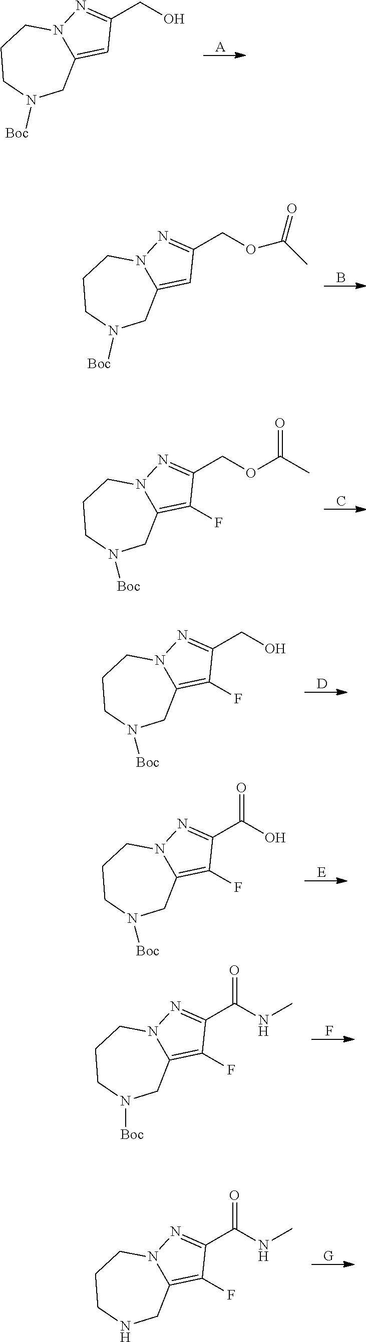

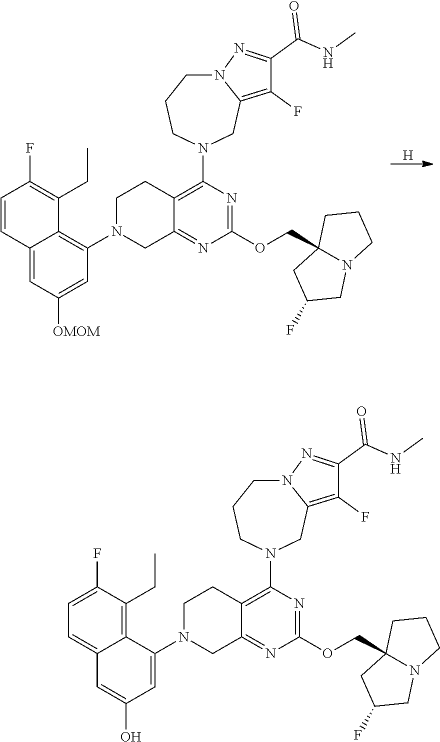

Step A. tert-butyl 2-(acetoxymethyl)-7,8-dihydro-4H-pyrazolo[1,5-a][1,4]diazepine-5(6H)-carboxylate: To a solution of tert-butyl 2-(hydroxymethyl)-7,8-dihydro-4H-pyrazolo[1,5-a][1,4]diazepine-5(6H)-carboxylate (20.0 g, 1.0 equiv) in THF (200 mL) were added TEA (22.7 g, 3.0 equiv) and acetyl chloride (11.8 g, 2.0 equiv). The mixture was stirred at 0° C. for 0.5 hours. The mixture was diluted with water (300 mL) and extracted with ethyl acetate (4×100 mL). The organic phase was dried over Na₂SO₄, concentrated, and purified by reversed phase flash chromatography [C18, 0.1% formic acid condition] to afford the title compound (16.0 g, 69% yield) as yellow oil; LCMS (ESI, M+1): m/z=310.2.

Step B. tert-butyl 2-(acetoxymethyl)-3-fluoro-7,8-dihydro-4H-pyrazolo[1,5-a][1,4]diazepine-5(6H)-carboxylate: To a solution of tert-butyl 2-(acetoxymethyl)-7,8-dihydro-4H-pyrazolo[1,5-a][1,4]diazepine-5(6H)-carboxylate (16.0 g, 1.0 equiv) in MeCN (160 mL) was added 1-(chloromethyl)-4-fluoro-1,4-diazoniabicyclo[2.2.2]octane;ditetrafluoroborate (91.6 g, 5.0 equiv). The mixture was stirred at 40° C. for 12 hours. The reaction was filtered and purified by reversed phase flash chromatography [C18, 0.1% formic acid condition] to afford the title compound (5.00 g, 29% yield) as yellow oil; LCMS (ESI, M+1): m/z=328.1.

Step C. tert-butyl 3-fluoro-2-(hydroxymethyl)-7,8-dihydro-4H-pyrazolo[1,5-a][1,4]diazepine-5(6H)-carboxylate: To a solution of tert-butyl 2-(acetoxymethyl)-3-fluoro-7,8-dihydro-4H-pyrazolo[1,5-a][1,4]diazepine-5(6H)-carboxylate (5.00 g, 1.0 equiv) in MeOH (10 mL) was added K2CO₃ (6.33 g, 3.0 equiv) in H₂O (5 mL). The mixture was stirred at 0° C. for 0.5 hours. The reaction was filtered and purified by reversed phase flash chromatography [C18, 0.1% formic acid condition] to afford the title compound (4.00 g, 90% yield) as yellow oil; LCMS (ESI, M+1): m/z=286.2

Step D. 5-(tert-butoxycarbonyl)-3-fluoro-5,6,7,8-tetrahydro-4H-pyrazolo[1,5-a][1,4]diazepine-2-carboxylic acid: To a solution of tert-butyl 3-fluoro-2-(hydroxymethyl)-7,8-dihydro-4H-pyrazolo[1,5-a][1,4]diazepine-5(6H)-carboxylate (600 mg, 1.0 equiv) in MeCN (6 mL) was added RuCl3·3H₂O (275 mg, 0.5 equiv) and then a solution of NaIO4 (1.35 g, 3.0 equiv) in H₂O (6 mL) was added dropwise. The mixture was stirred at 20° C. for 1 hour. The mixture was quenched with saturated Na2SO3 aqueous (20 mL) at 0° C. and extracted with ethyl acetate (4×20 mL). The combined organic layers were washed with saturated brine 20 mL, dried over Na₂SO₄, filtered, and purified by reversed phase flash chromatography [C18, 0.1% formic acid condition] to afford the title compound (200 mg, 31% yield) as yellow solid; LCMS (ESI, M+1): m/z=300.2.

Step E. tert-butyl 3-fluoro-2-(methylcarbamoyl)-7,8-dihydro-4H-pyrazolo[1,5-a][1,4]diazepine-5(6H)-carboxylate: To a solution of 5-(tert-butoxycarbonyl)-3-fluoro-5,6,7,8-tetrahydro-4H-pyrazolo[1,5-a][1,4]diazepine-2-carboxylic acid (200 mg, 1.0 equiv) and methanamine (226 mg, 5.0 equiv, HCl) in THF (2 mL) were added TEA (541 mg, 8.0 equiv) and HATU (508 mg, 2.0 equiv). The mixture was stirred at 40° C. for 2 hours. The mixture was diluted with water (15 mL) and extracted with ethyl acetate (4×15 mL). The organic phase was dried over Na₂SO₄, concentrated, and purified by reversed phase flash chromatography [C18, 0.1% formic acid condition] to afford the title compound (110 mg, 52% yield) as yellow solid; LCMS (ESI, M+1): m/z=313.1.

Step F. 3-fluoro-N-methyl-5,6,7,8-tetrahydro-4H-pyrazolo[1,5-a][1,4]diazepine-2-carboxamide: To a solution of tert-butyl 3-fluoro-2-(methylcarbamoyl)-7,8-dihydro-4H-pyrazolo[1,5-a][1,4]diazepine-5(6H)-carboxylate (110 mg, 1.0 equiv) in MeCN (0.5 mL) was added HCl•dioxane (4 M, 0.5 mL, 5.7 equiv). The mixture was stirred at 0° C. for 0.5 hours. The reaction was concentrated under reduced pressure to give a residue. The residue was slurried with MeOH (10 mL) and NaHCO₃solid (300 mg). The residue was filtered and concentrated to afford the title compound (50 mg, crude) as white solid.

The last two steps were performed according to Example 248. The title compound was obtained as yellow solid. ¹H NMR (400 MHz, METHANOL-d4) δ=7.52-7,48 (m, 1H), 7.16-7.11 (m, 1H), 6.97-6.96 (m, 2H), 5.35-5.17 (m, 1H), 4.85-4.81 (m, 2H), 4.55-4.39 (m, 2H), 4.14-3.96 (m, 5H), 3.66 (br d, J=17.6 Hz, 1H), 3.52-3.50 (m, 1H), 3.42-3.35 (m, 2H), 3.26-3.09 (m, 5H), 3.02-2.92 (m, 1H), 2.87 (s, 3H), 2.70 (br d, J=14.4 Hz, 1H), 2.32-2.16 (m, 2H), 2.15-1.99 (m, 3H), 1.98-1.79 (m, 3H), 1.10 (t, J=7.2 Hz, 3H); LCMS (ESI, M+1): m/z=691.4.

### Example 363

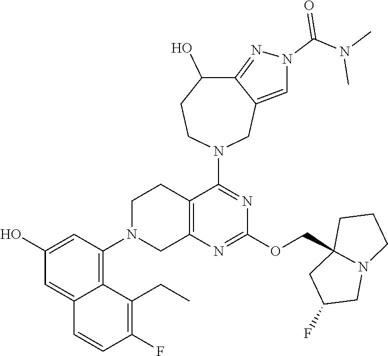 [SMILES: CCc1c(F)ccc2cc(O)cc(N3CCc4c(nc(OC[C@@]56CCCN5C[C@H](F)C6)nc4N4CCC(O)c5nn(C(=O)N(C)C)cc5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-8-hydroxy-N,N-dimethyl-5,6,7,8-tetrahydropyrazol o[4,3-c]azepine-2(4H)-carboxamide

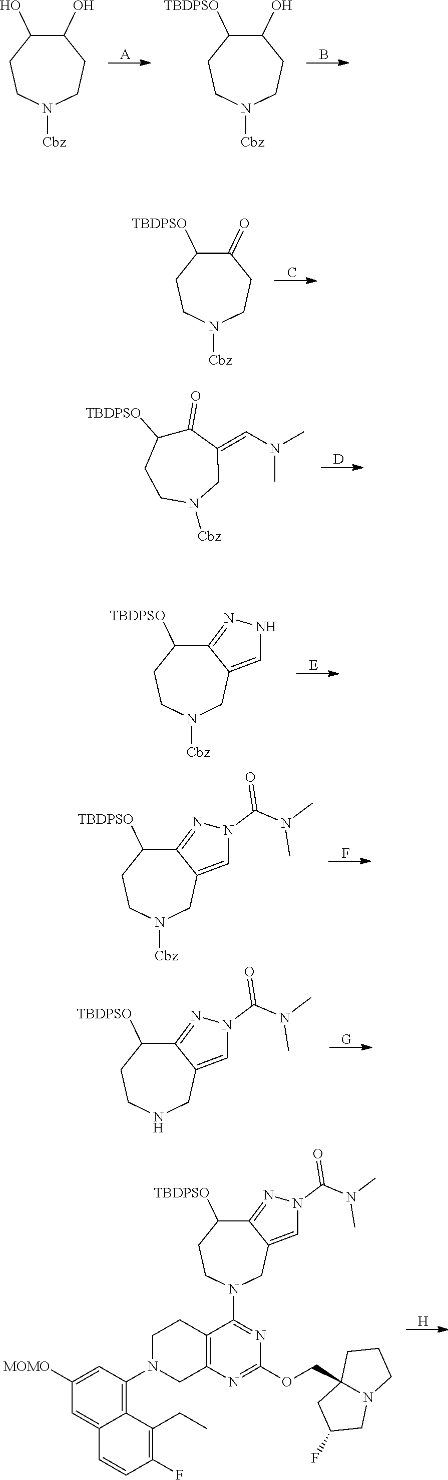

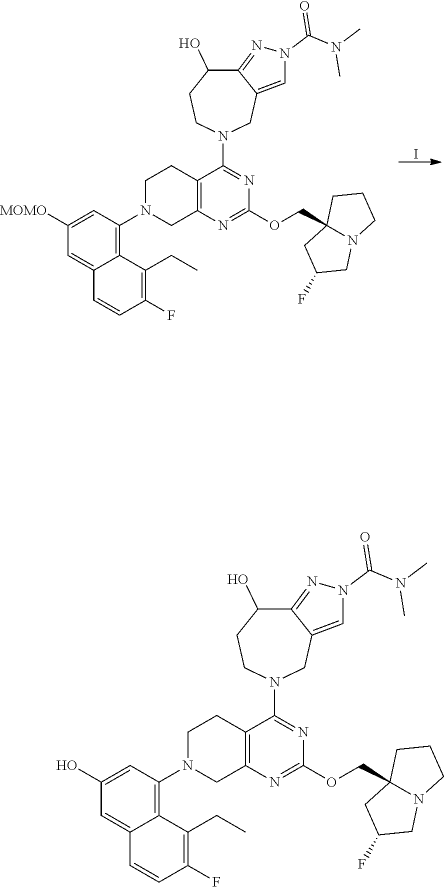

Step A. benzyl 4-((tert-butyldiphenvlsilyl)oxy)-5-hydroxyazepane-1-carboxylate: To a solution of benzyl 4,5-dihydroxyazepane-1-carboxylate (1.50 g, 1.0 equiv), imidazole (1.15 g, 3.0 equiv), and DMAP (6.91 mg, 0.01 equiv) in DCM (1 mL) was added TBDPS-C1 (1.09 g, 0.7 equiv) at 0° C. The reaction was stirred at 25° C. for 2 hours. The mixture was diluted with water (20 mL) and extracted with ethyl acetate (4×20 mL). The combined organic layers were dried over anhydrous sodium sulfate, concentrated, and purified by column chromatography [SiO₂, Petroleum ether/Ethyl acetate=3/1 to 1/1] to afford the title compound (1.20 g, 41% yield) as yellow liquid; 1H NMR (400 MHz, CHLOROFORM-d) δ=7.66-7.17 (m, 15H), 5.12-4.93 (m, 2H), 3.82-3.79 (m, 2H), 3.67-3.43 (m, 1H), 3.38-3.06 (m, 3H), 2.29-2.19 (m, 1H), 2.11-2.01 (m, 1H), 1.97-1.87 (m, 1H), 1.62-1.39 (m, 2H), 1.03 (br d, J=2.0 Hz, 9H); LCMS (ESI, M+1): m/z=504.3.

Step B. benzyl 4-((tert-butyldiphenylsilyl) oxy)-5-oxoazepane-1-carboxylate: To a solution of benzyl 4-((tert-butyldiphenylsilyl) oxy)-5-hydroxyazepane-1-carboxylate (1.20 g, 1.0 equiv) in DCM (12 mL) was added Dess-Martin (3.03 g, 3.0 equiv). The mixture was stirred at 25° C. for 2 hours. The reaction was filtered. The filtrate was diluted with water (15 mL) and extracted with ethyl acetate (4×15 mL). The combined organic layers were dried over anhydrous sodium sulfate, concentrated, and purified by column chromatography [SiO₂, Petroleum ether/Ethyl acetate=5/1] to afford the title compound (900 mg, 75% yield) as white liquid; ¹H NMR (400 MHz, CHLOROFORM-d) δ=7.57-7.44 (m, 15H), 5.05-4.94 (m, 2H), 4.41-4.27 (m, 1H), 3.95-3.57 (m, 3H), 3.46-3.25 (m, 1H), 2.80-2.40 (m, 2H), 1.71-1.41 (m, 2H), 1.05 (s, 9H).

Step C. benzyl (E)-5-((tert-butyldiphenvlsilyl)oxy)-3-((dimethylamino)methylene)-4-oxoazepane-1-carboxylate: To a solution of benzyl 4-[tert-butyl(diphenyl)silyl]oxy-5-oxo-azepane-1-carboxylate (2.00 g, 1.0 equiv) in THF (25 mL) was added 1-tert-butoxy-N,N,N′,N′-tetramethyl-methanediamine (834 mg, 1.2 equiv). The reaction was stirred at 60° C. for 8 hours. The mixture was diluted with water (10 mL) and extracted with ethyl acetate (4×10 mL). The organic phase was dried over anhydrous sodium sulfate and concentrated to afford the title compound (2.00 g, crude) as yellow oil.

Step D. benzyl 8-((tert-butyldiphenvlsilyl)oxy)-4,6,7,8-tetrahydropyrazolo[4,3-c]azepine-5(2H)-carboxylate: To a solution of benzyl (E)-5-((tert-butyldiphenylsilyl)oxy)-3-((dimethylamino)methylene)-4-oxoazepane-1-carboxylate (200 mg) in EtOH (3 mL) was added N2H4H₂O (630 mg, 85% purity). The mixture was stirred at 75° C. for 12 hours. The reaction was diluted with water (5 mL) and extracted with ethyl acetate (4×5 mL). The organic phase was dried over anhydrous sodium sulfate, concentrated, and purified by reversed phase flash chromatography [C18, 0.1% formic acid condition] to afford the title compound (20.0 mg, 10% yield) as white oil; ¹H NMR (400 MHz, CHLOROFORM-d) δ=7.60-7.20 (m, 16H), 5.08-4.88 (m, 3H), 4.78-4.48 (m, 1H), 4.29-4.08 (m, 1H), 3.86 (br d, J=6.4 Hz, 2H), 1.88-1.61 (m, 2H), 0.97-0.96 (m, 9H).

Step E. benzyl 8-((tert-butyldiphenvlsilyl)oxy)-2-(dimethylcarbamoyl)-4,6,7,8-tetrahydropyrazolo[4,3-c]azepine-5(2H)-carboxylate: To a solution of benzyl 8-((tert-butyldiphenylsilyl)oxy)-4,6,7,8-tetrahydropyrazolo[4,3-c]azepine-5(2H)-carboxylate (10.0 mg, 1.0 equiv) in THE (0.5 mL) was added NaH (2.28 mg, 60% purity, 3.0 equiv) at 0° C. The mixture was stirred at 0° C. for 0.5 hours. Then dimethylcarbamic chloride (4.09 mg, 2.0 equiv) was added into the mixture. The mixture was stirred at 0° C. for 0.5 hours. The mixture was diluted with water (5 mL) and extracted with ethyl acetate (4×5 mL). The organic phase was dried over anhydrous sodium sulfate, concentrated, and purified by reversed phase flash chromatography [C18, 0.1% formic acid condition] to afford the title compound (10.0 mg, crude) as yellow oil; ¹H NMR (400 MHz, CiLOROFORM-d) δ=7.96-7.75 (m, 1H), 7.61-7.60 (m, 2H), 7.40-7.11 (m, 13H), 5.09-4.75 (m, 4H), 4.19-4.07 (m, 1H), 4.03-3.97 (m, 1H), 3.87-3.69 (m, 1H), 2.91 (s, 6H), 1.89-1.79 (m, 1H), 1.77-1.62 (m, 1H), 0.96 (br s, 9H); LCMS (ESI, M+1): m/z=597.4.

Step F. 8-((tert-butyldiphenylsilyl)oxy)-N,N-dimethyl-5,6,7,8-tetrahydropyrazolo[4,3-c]azepine-2(4H)-carboxamide: To a solution of benzyl 8-((tert-butyldiphenylsilyl)oxy)-2-(dimethylcarbamoyl)-4,6,7,8-tetrahydropyrazolo[4,3-c]azepine-5(2H)-carboxylate (200 mg, 1.0 equiv) in MeOH (2 mL) was added Pd/C (50.0 mg, 10% purity) under N2 atmosphere. The suspension was degassed and purged with H2 3 times. The reaction was stirred under H2 (15 Psi) at 25° C. for 2 hours. The reaction was filtered and concentrated to afford the title compound (300 mg, crude) as yellow oil.

Step G. 8-((tert-butyldiphenvlsilyl)oxy)-5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-5,6,7,8-tetrahydropyrazolo[4,3-c]azepine-2(4H)-carboxamide: To a solution of 7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (150 mg, 1.0 equiv) and 8-((tert-butyldiphenylsilyl)oxy)-N,N-dimethyl-5,6,7,8-tetrahydropyrazolo[4,3-c]azepine-2(4H)-carboxamide (150 mg, 1.5 equiv) in DMF (0.5 mL) were added N,N-diethylpropan-2-amine (83.7 mg, 3.0 equiv) and 4A molecular sieve (50 mg). The mixture was stirred at 40° C. for 24 hours. The reaction was filtered and purified by reversed phase flash chromatography [C18, 0.1% formic acid condition] to afford the title compound (120 mg, 53% yield) as yellow solid; LCMS (ESI, M+1): m/z=985.7.

Step H. 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-8-hydroxy-N,N-dimethyl-5,6,7,8-tetrahydropyrazolo[4,3-c]azepine-2(4H)-carboxamide: To a solution of 8-((tert-butyldiphenylsilyl)oxy)-5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-5,6,7,8-tetrahydropyrazolo[4,3-c]azepine-2(4H)-carboxamide (60.0 mg, 1.0 equiv) in DMF (0.5 mL) was added CsF (92.5 mg, 10 equiv). The mixture was stirred at 20° C. for 24 hours. The reaction was filtered and purified by reversed phase flash chromatography [C18, 0.1% formic acid condition] to afford the title compound (30.0 mg, 66% yield) as white solid; LCMS (ESI, M+1): m/z=747.5.

Step I. 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-8-hydroxy-N,N-dimethyl-5,6,7,8-tetrahydropyrazolo[4,3-c]azepine-2(4H)-carboxamide: To a solution of 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-8-hydroxy-N,N-dimethyl-5,6,7,8-tetrahydropyrazolo[4,3-c]azepine-2(4H)-carboxamide (30.0 mg, 1.0 equiv) in MeOH (1 mL) was added HCl•MeOH (4 M, 1 mL, 1.0 equiv). The mixture was stirred at 0° C. for 0.5 hours. The mixture was quenched with saturated NaHCO₃ aqueous (5 mL) at 0° C. and extracted with EtOAc (4×5 mL). The combined organic layers were dried over anhydrous Na₂SO₄, concentrated, and purified with prep-HPLC [column: Waters Xbridge 150 x 25 mm×5 μm; A: water (10 mM NH₄HCO₃), B: ACN, B %: 43%-73% over 8 min] and prep-HPLC [column: Phenomenex luna C18 150×25 mm×10 μm; A: water (FA), B: ACN, B %: 14%-44% over 2 min] to afford the title compound (13.4 mg, 47% yield) as white solid; ¹H NMR (400 MHz, METHANOL-d4) δ=8.13-8.08 (m, 1H), 7.52-7.49 (m, 1H), 7.16-7.11 (m, 1H), 6.96 (d, J=2.8 Hz, 2H), 5.44-5.21 (m, 1H), 5.18-4.95 (m, 2H), 4.89 (br s, 1H), 4.62-4.57 (im, 1H), 4.49-4.37 (m, 1H), 4.31-4.07 (m, 3H), 4.05-3.95 (m, 1H), 3.65 (d, J=17.2 Hz, 1H), 3.54-3.51 (m, 1H), 3.40-3.31 (m, 5H), 3.24-3.17 (m, 6H), 3.17-3.02 (m, 2H), 2.77-2.74 (m, 1H), 2.41-2.13 (m, 4H), 2.10 (br s, 4H), 1.09 (br t, J=7.2 Hz, 3H), LCMS (ESI, M+1): m/z=703.4.

### Example 364

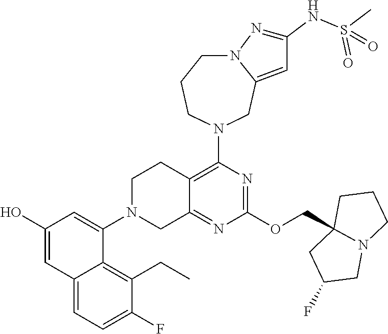 [SMILES: CCc1c(F)ccc2cc(O)cc(N3CCc4c(nc(OC[C@@]56CCCN5C[C@H](F)C6)nc4N4CCCn5nc(NS(C)(=O)=O)cc5C4)C3)c12]

### N-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanesulfonamide

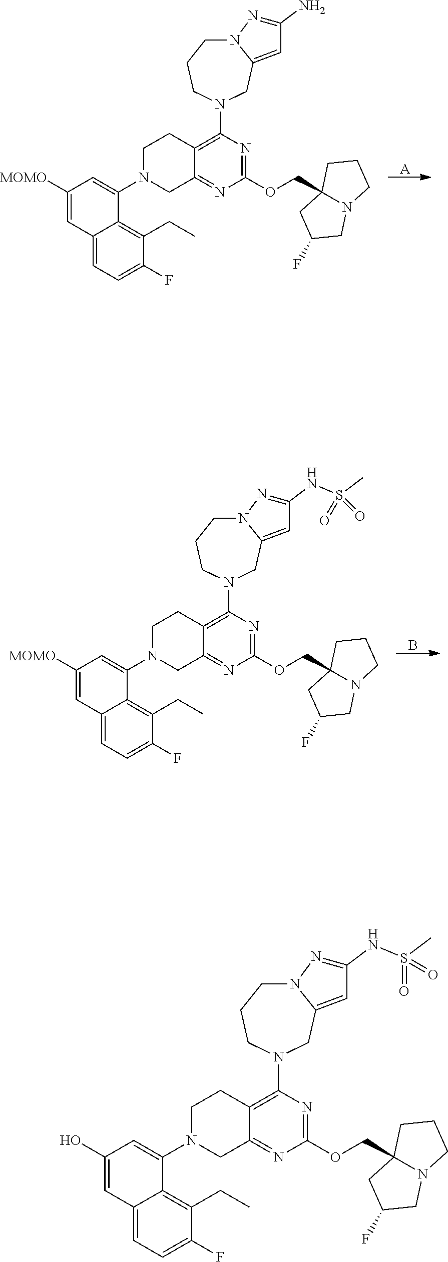

Step A. N-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5.6,7,8-tetrahydropyrido[3,4-d]pyrimidin-4-yl)-5,6,7,8-tetrahydro-4H-pyrazolo[1,5-a][1,4]diazepin-2-yl)methanesulfonamide: To a mixture of 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-amine (300 mg, 1.0 equiv) in pyridine (4 mL) was added Ms20 (116 mg, 1.5 equiv) in an ice-bath. The reaction was warmed to 20° C. and stirred for 4 hours. The mixture was diluted with water (10 mL) and extracted with EtOAc (3×20 mL). The combined organic layers were dried over anhydrous sodium sulfate, concentrated, and purified by reversed phase flash chromatography [C18, 0.1% formic acid condition] and lyophilized to afford the title compound (300 mg, 86% yield) as yellow solid; LCMS (ESI, M+1): m/z=753.2.

Step B. N-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanesulfonamide: To a mixture of N-(5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanesulfonamide (143 mg, 1.0 equiv) in MeCN (0.35 mL) was added HCl•MeOH (4 M, 712 μL, 15 equiv). The reaction was stirred at 20° C. for 1 hour. The mixture was filtered and purified with prep-HPLC [Phenomenex luna C18 150×25 mm×10 μm; A: water (FA), B: ACN, B %: 16%-46% over 10 min] and lyophilized to afford the title compound (18.5 mg) as orange solid (FA salt). ¹H NMR (400 MHz, dimethylsulfoxide-d6) δ=9.76 (br s, 1H), 9.68 (s, 1H), 7.59 (dd, J=6.0, 9.2 Hz, 1H), 7.24 (t, J=9.2 Hz, 1H), 6.99 (s, 2H), 5.97 (s, 1H), 5.38-5.17 (m, 1H), 4.93 (br d, J=16.0 Hz, 1H), 5.01-4.85 (m, 1H), 4.68 (br d, J=16.0 Hz, 1H), 4.35 (br d, J=5.6 Hz, 2H), 4.17-3.84 (m, 4H), 3.77 (br d, J=11.6 Hz, 2H), 3.49-3.38 (m, 2H), 3.17-3.03 (m, 5H), 3.00 (s, 3H), 2.91-2.79 (m, 1H), 2.22-2.09 (m, 2H), 2.08-1.87 (m, 4H), 1.86-1.66 (m, 3H), 1.12-1.01 (m, 3H); ¹⁹F NMR (400 MHz, dimethylsulfoxide-d6) 8=−121.311, -172.069; LCMS (ESI, M+1): m/z=709.6.

### Example 365

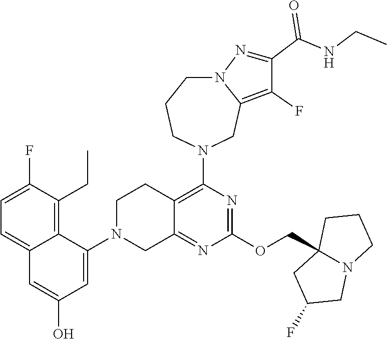 [SMILES: CCNC(=O)c1nn2c(c1F)CN(c1nc(OC[C@@]34CCCN3C[C@H](F)C4)nc3c1CCN(c1cc(O)cc4ccc(F)c(CC)c14)C3)CCC2]

### N-ethyl-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-3-fluoro-5,6,7,8-tetrahydro-4H-pyrazolo[1 5-a][14]diazepine-2-carboxamide

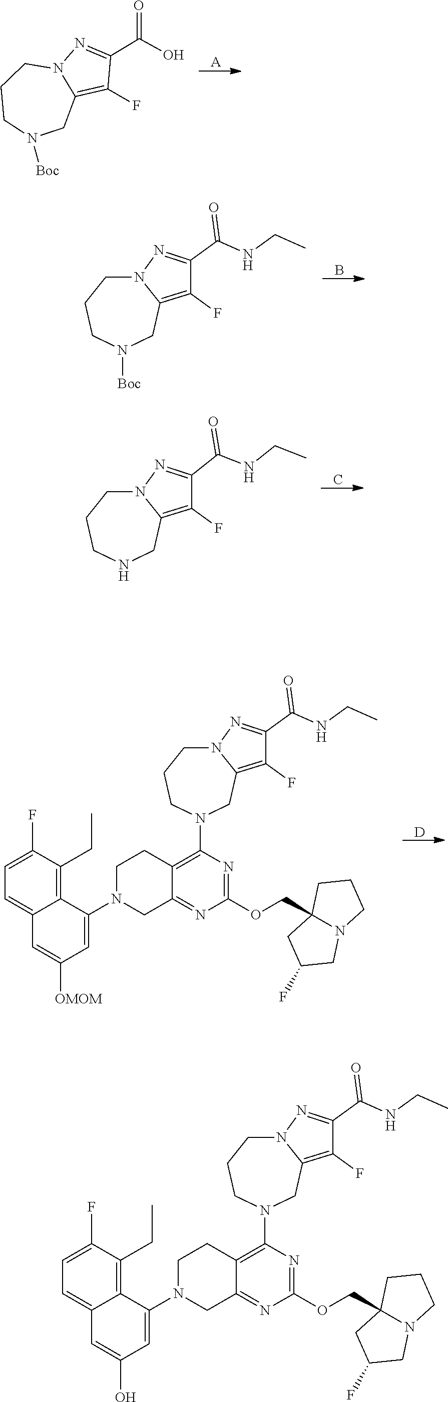

Step A. tert-butyl 2-(ethylcarbamoyl)-3-fluoro-7,8-dihydro-4H-pyrazolo[1,5-a][1,4]diazepine-5(6H)-carboxylate: To a solution of 5-(tert-butoxycarbonyl)-3-fluoro-5,6,7,8-tetrahydro-4H-pyrazolo[1,5-a][1,4]diazepine-2-carboxylic acid (100 mg, 1.0 equiv), TEA (101 mg, 3.0 equiv), and HATU (254 mg, 2.0 equiv) in THE (1 mL) was added ethanamine (112 mg, 67% purity, 5.0 equiv). The reaction was stirred at 40° C. for 12 hours. The reaction was filtered and purified by reversed phase flash chromatography [C18, 0.1% formic acid condition] to afford the title compound (40.0 mg, 36% yield) as yellow solid; LCMS (ESI, M+1): m/z=327.2.

Step B. N-ethyl-3-fluoro-5,6,7,8-tetrahydro-4H-pyrazolo[1,5-a][1,4]diazepine-2-carboxamide: To a solution of tert-butyl 2-(ethylcarbamoyl)-3-fluoro-7,8-dihydro-4H-pyrazolo[1,5-a][1,4]diazepine-5(6H)-carboxylate (40 mg, 1.0 equiv) in MeCN (0.5 mL) was added HCl•dioxane (4 M, 0.5 mL, 11.0 equiv) at 0° C. The reaction was stirred at 0° C. for 0.5 hours. The mixture was concentrated to afford the title compound (40.0 mg, HCl, crude) as yellow solid.

The last two steps were performed according to Example 248. The title compound was obtained as pink solid (FA salt). ¹H NMR (400 MHz, METHANOL-d4) δ=7.51 (dd, J=5.6, 8.8 Hz, 1H), 7.14 (t, J=9.6 Hz, 1H), 6.97 (br d, J=4.0 Hz, 2H), 5.47-5.21 (m, 1H), 4.90 (br s, 1H), 4.84-4.77 (m, 1H), 4.57-4.40 (m, 2H), 4.24-3.97 (m, 5H), 3.67 (br d, J=17.6 Hz, 1H), 3.52 (br d, J=9.6 Hz, 1H), 3.43-3.32 (m, 6H), 3.30-3.12 (m, 3H), 3.12-3.03 (m, 1H), 2.72 (br d, J=14.4 Hz, 1H), 2.42-2.11 (m, 4H), 2.08-1.86 (m, 4H), 1.19 (t, J=7.2 Hz, 3H), 1.10 (t, J=7.2 Hz, 3H); LCMS (ESI, M+1): m/z=705.4.

### Example 366

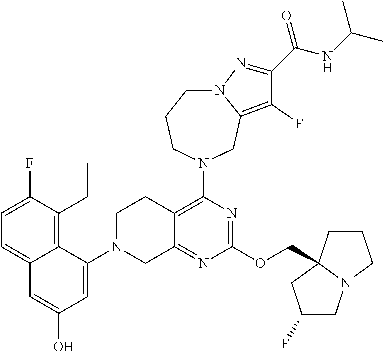 [SMILES: CCc1c(F)ccc2cc(O)cc(N3CCc4c(nc(OC[C@@]56CCCN5C[C@H](F)C6)nc4N4CCCn5nc(C(=O)NC(C)C)c(F)c5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-3-fluoro-N-isopropyl-5,6,7,8-tetrahydro-4H-pyrazolo[1,5-a][1,4]diazepine-2-carboxamide

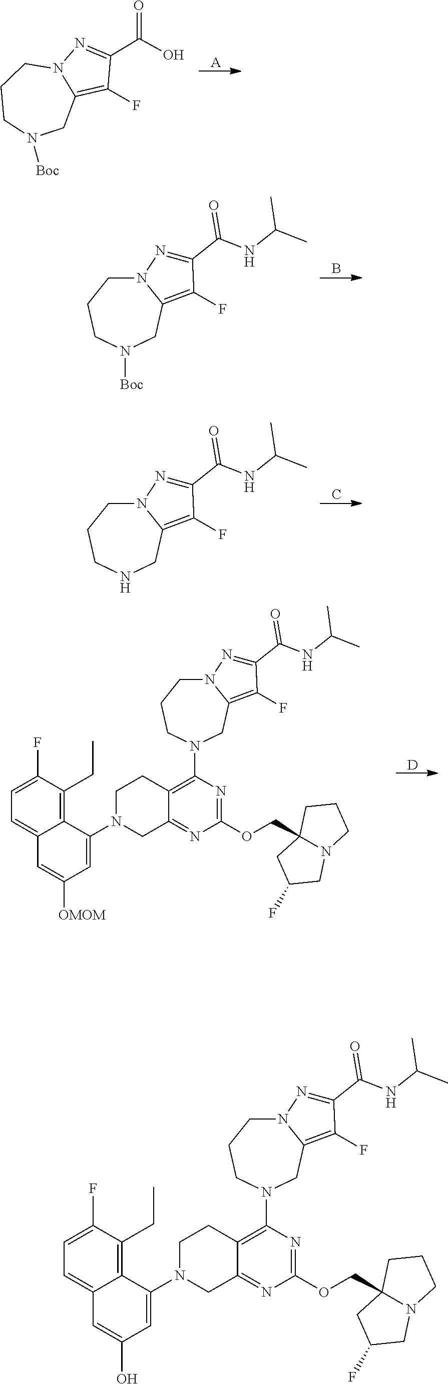

Synthesized according to Example 365. The title compound was obtained as white solid (FA salt). 1H NMR (400 MHz, METHANOL-d4) δ=7.52-7.48 (m, 1H), 7.14 (t, J=9.4 Hz, 1H), 6.97 (d, J=3.6 Hz, 2H), 5.46-5.28 (m, 1H), 4.82 (s, 1H), 4.48 (s, 2H), 4.26-4.12 (m, 4H), 4.11-4.01 (m, 2H), 3.66 (d, J=17.6 Hz, 1H), 3.55-3.44 (m, 2H), 3.43-3.35 (m, 4H), 3.28-3.07 (m, 3H), 2.72 (d, J=14.4 Hz, 1H), 2.50-2.12 (m, 5H), 2.10-1.87 (m, 4H), 1.23 (d, J=6.4 Hz, 6H), 1.09 (t, J=7.2 Hz, 3H); LCMS (ESI, M+1): m/z=719.5 o

### Example 367

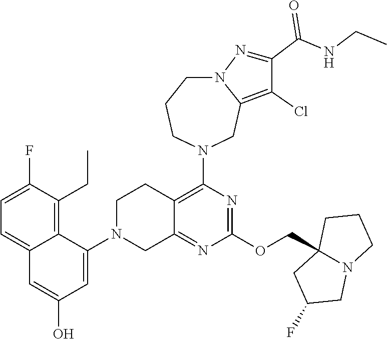 [SMILES: CCNC(=O)c1nn2c(c1Cl)CN(c1nc(OC[C@@]34CCCN3C[C@H](F)C4)nc3c1CCN(c1cc(O)cc4ccc(F)c(CC)c14)C3)CCC2]

### 3-chloro-N-ethyl-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepine-2-carboxamide

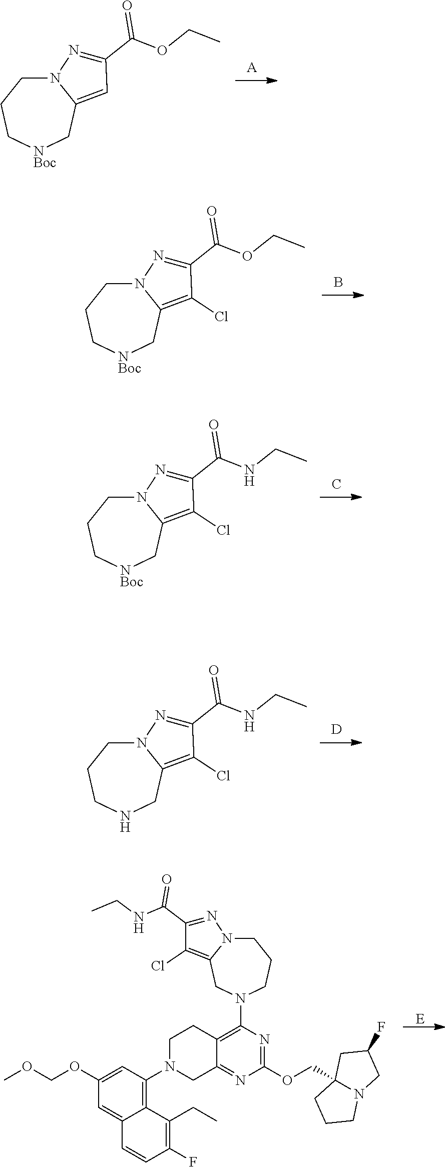

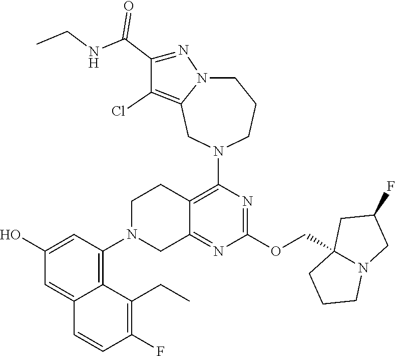 [SMILES: CCNC(=O)c1nn2c(c1Cl)CN(c1nc(OC[C@@]34CCCN3C[C@H](F)C4)nc3c1CCN(c1cc(O)cc4ccc(F)c(CC)c14)C3)CCC2]

Step A. 5-tert-butyl 2-ethyl 3-chloro-7,8-dihydro-4H-pyrazolo[1,5-a][1,4]diazepine-2,5(6H)-dicarboxylate: To a mixture of 5-tert-butyl 2-ethyl 7,8-dihydro-4H-pyrazolo[1,5-a][1,4]diazepine-2,5(6H)-dicarboxylate (800 mg, 1.0 equiv) in DMF (8.0 mL) was added 1-chloropyrrolidine-2,5-dione (518 mg, 1.5 equiv). The reaction was stirred at 55° C. for 1 hour. The mixture was diluted with H₂O (10 mL) and extracted with EtOAc (3×10 mL). The combined organic layers were washed with NaHCO₃ (3×10 mL), dried over anhydrous sodium sulfate, and concentrated to afford the title compound (888 mg, 99% yield) as colorless oil; 1H NMR (400 MHz, dimethylsulfoxide-d6) δ=4.54 (br s, 2H), 4.52-4.48 (m, 2H), 4.44 (q, J=7.2 Hz, 2H), 3.77 (br s, 2H), 1.99 (br s, 2H), 1.49-1.39 (m, 12H); LCMS (ESI, M+1): m/z=344.2.

Step B. tert-butyl 3-chloro-2-(ethylcarbamoyl)-7,8-dihydro-4H-pyrazolo[1,5-a][1,4]diazepine-5(6H)-carboxylate: To a mixture of ethanamine (592 mg, 12 equiv) in toluene (5.0 mL) was added trimethylaluminum (2.0 M, 113 mg, 2.0 equiv). The mixture was stirred at 0° C. for 0.5 hours, and then 5-tert-butyl 2-ethyl 3-chloro-7,8-dihydro-4H-pyrazolo[1,5-a][1,4]diazepine-2,5(6H)-dicarboxylate (376 mg, 1.0 equiv) was added. The reaction was stirred at 80° C. for 2 hours. The mixture was diluted with saturated sodium sulfate aqueous solution (1.0 mL) and extracted with THE (3×5.0 mL). The mixture was filtered, concentrated, and purified by reversed phase chromatography [C18, 0.1% formic acid condition] to afford the title compound (131 mg, 32% yield) as yellow solid; 1H NMR (400 MHz, chloroform-d) δ=6.73 (br s, 1H), 4.51 (br s, 2H), 4.43-4.35 (m, 2H), 3.75 (br s, 2H), 3.53-3.39 (m, 2H), 1.97 (br s, 2H), 1.44 (s, 9H), 1.27-1.21 (m, 3H).

Step C. 3-chloro-N-ethyl-5,6,7,8-tetrahydro-4H-pyrazolo[1,5-a][1,4]diazepine-2-carboxamide: To a mixture of tert-butyl 3-chloro-2-(ethylcarbamoyl)-7,8-dihydro-4H-pyrazolo[1,5-a][1,4]diazepine-5(6H)-carboxylate (131 mg, 1.0 equiv) in ACN (0.5 mL) was added HCl•dioxane (4 M, 2.0 mL, 21 equiv). The reaction was stirred at 20° C. for 0.5 hours. The mixture was concentrated to afford the title compound (211 mg, crude, HCl) as yellow solid; LCMS (ESI, M+1): m/z=243.2.

The last two steps were performed according to Example 248. The title compound was obtained as orange solid (FA salt). ¹H NMR (400 MHz, methanol-d4) δ=7.51 (dd, J=6.0, 8.8 Hz, 1H), 7.15 (t, J=9.2 Hz, 1H), 7.03-6.90 (m, 2H), 5.51-5.30 (m, 1H), 4.83 (s, 2H), 4.56-4.47 (m, 11H), 4.39 (td, J=7.2, 14.0 Hz, 11H), 4.24 (dd, J=9.2, 11.2 Hz, 11H), 4.16-4.01 (m, 4H), 3.67 (br d, J=18.0 Hz, 1H), 3.59-3.44 (m, 4H), 3.44-3.33 (m, 5H), 3.23-3.11 (m, 2H), 2.73 (br d, J=15.2 Hz, 1H), 2.53-2.25 (m, 3H), 2.24-2.08 (m, 4H), 2.03-1.88 (m, 1H), 1.20 (t, J=7.2 Hz, 3H), 1.11 (t, J=7.2 Hz, 3H); ¹⁹F NMR (400 MHz, methanol -d4) δ=−123.037, -173.651; LCMS (ESI, M+1): m/z=721.2.

### Example 368

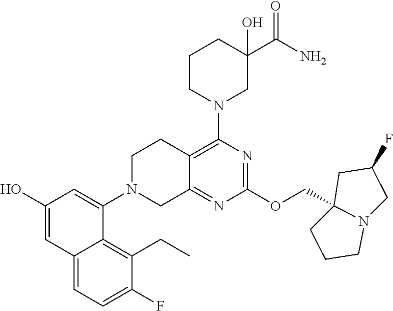 [SMILES: CCc1c(F)ccc2cc(O)cc(N3CCc4c(nc(OC[C@@]56CCCN5C[C@H](F)C6)nc4N4CCCC(O)(C(N)=O)C4)C3)c12]

### 1-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-3-hydroxypiperidine-3-carboxamide

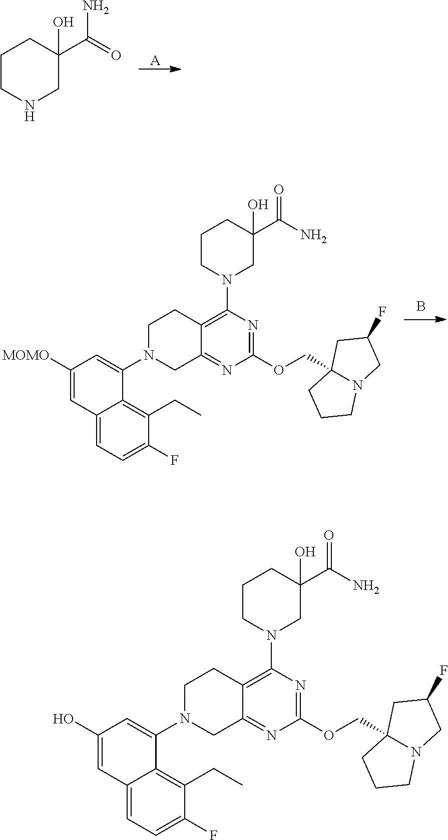

Synthesized according to Example 248. The title compound was obtained as white solid (FA salt). ¹H NMR (400 MHz, METHANOL-d4) δ=8.54-8.42 (m, 1H), 7.51 (dd, J=6.0, 9.0 Hz, 1H), 7.15 (t, J=9.6 Hz, 1H), 7.01-6.93 (m, 2H), 5.52-5.35 (m, 1H), 4.29 (br d, J=2.0 Hz, 2H), 4.27-4.13 (m, 1H), 4.07 (br d, J=17.2 Hz, 2H), 3.74-3.55 (m, 4H), 3.53-3.46 (m, 2H), 3.45-3.33 (m, 2H), 3.30-3.12 (m, 3H), 3.10-2.94 (m, 1H), 2.87-2.64 (m, 1H), 2.57-2.35 (m, 2H), 2.32-2.15 (m, 4H), 2.13-1.88 (m, 2H), 1.85-1.68 (m, 2H), 1.17-1.06 (m, 3H); LCMS (ESI, M+1). m/z=623.4.

### Example 369

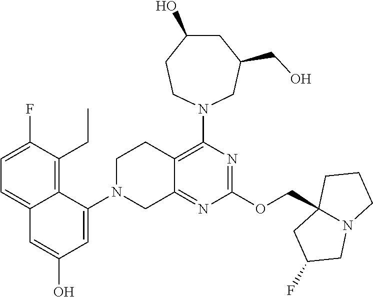 [SMILES: CCc1c(F)ccc2cc(O)cc(N3CCc4c(nc(OC[C@@]56CCCN5C[C@H](F)C6)nc4N4CC[C@@H](O)C[C@@H](CO)C4)C3)c12]

### (4S,6R)-1-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-6-(hydroxymethyl)azepan-4-ol

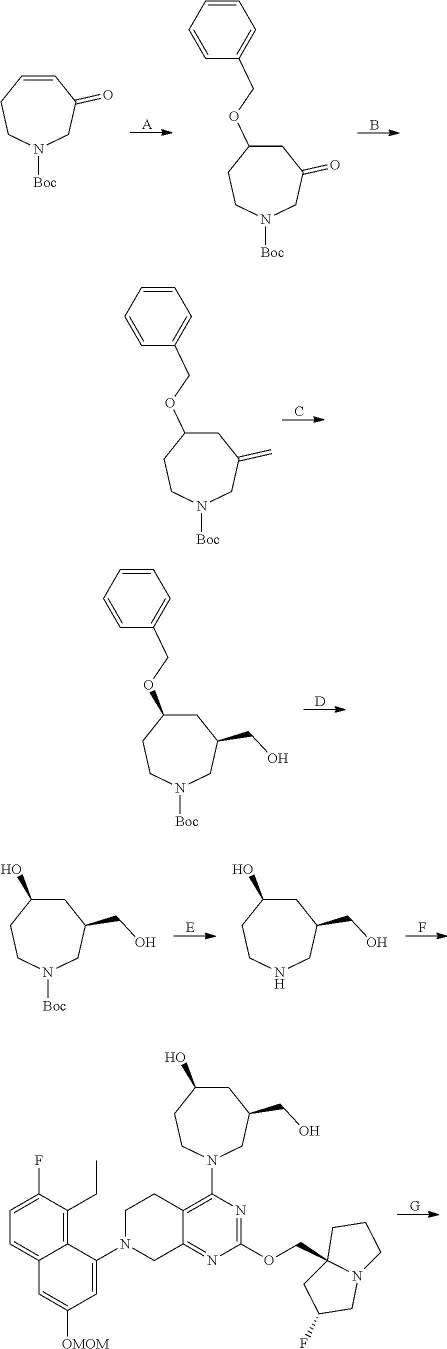

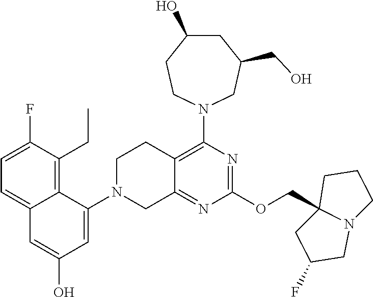 [SMILES: CCc1c(F)ccc2cc(O)cc(N3CCc4c(nc(OC[C@@]56CCCN5C[C@H](F)C6)nc4N4CC[C@@H](O)C[C@@H](CO)C4)C3)c12]

Step A. tert-butyl 5-(benzyloxy)-3-oxoazepane-1-carboxylate: To a solution of tert-butyl 3-oxo-2,3,6,7-tetrahydro-1H-azepine-1-carboxylate (2.30 g, 1.0 equiv) in phenylmethanol (14.3 g, 12 equiv) was added tBuONa (105 mg, 0.10 equiv) at 0° C. The mixture was stirred at 20° C. for 2 hours. The mixture was diluted with water (10 mL) and extracted with ethyl acetate (2×10 mL). The organic layer was washed with brine (10 mL) and dried over Na₂SO₄. The organic layer was concentrated and purified by reversed phase flash chromatography [C18, 0.1% formic acid condition] to afford the title compound (3.00 g, 85% yield) as yellow oil; LCMS (ESI, M+1): m/z=220.2.

Step B. tert-butyl 5-(benzyloxy)-3-methyleneazepane-1-carboxylate: To a mixture of methyl(triphenyl)phosphonium;bromide (8.39 g, 2.5 equiv) in THE (15 mL) was added n-BuLi (10.5 mL, 2.8 equiv) at 0° C. The mixture was stirred at 0° C. for 0.5 hours. To the mixture was added tert-butyl 5-(benzyloxy)-3-oxoazepane-1-carboxylate (3.00 g, 1.0 equiv) at 0° C. The mixture was stirred at 0° C. for 1 hour. The mixture was quenched by saturated NH₄Cl (10 mL) and extracted with ethyl acetate (2×10 mL). The organic layer was washed with brine (10 mL) and dried over Na₂SO₄. The solvent was concentrated and purified by column chromatography [SiO₂, Petroleum ether/Ethyl acetate=50/1 to 2/1] to afford the title compound (1.4 g, 37% yield) as yellow oil; ¹H NMR (400 MHz, METHANOL-d4) δ=7.44-7.26 (m, 4H), 5.08-4.91 (m, 2H), 4.84-4.42 (m, 3H), 4.23-4.08 (m, 1H), 4.04-3.84 (m, 1H), 3.67-3.38 (m, 2H), 3.22-3.05 (m, 1H), 2.61-2.25 (m, 2H), 2.03-1.67 (m, 2H), 1.46 (d, J=6.8 Hz, 9H); LCMS (ESI, M+1): m/z=218.2

Step C. (3R,5S)-tert-butyl 5-(benzyloxy)-3-(hydroxymethyl)azepane-1-carboxylate: To a solution of tert-butyl 5-benzyloxy-3-methylene-azepane-1-carboxylate (1.00 g, 1.0 equiv) in THE (8 mL) was added BH3Me2S (945 L, 3.0 equiv) at 0° C. The mixture was stirred at 0° C. for 1.5 hours. To the mixture were added H₂O₂ (13.0 g, 30% purity, 36 equiv) and NaOH (4.2 mL, 4.0 equiv). The mixture was stirred at 0° C. for 1 hour and then at 25° C. for 9 hours. The mixture was quenched by saturated Na2SO3 and extracted with ethyl acetate (2×10 mL) The organic layer was washed with brine (10 ml) and dried over Na₂SO₄. The solvent was concentrated to dryness. The crude product was purified by reversed phase flash chromatography [C18, 0.1% formic acid condition] and SFC [column: DAICEL CHIRALCEL OJ-H (250 mm×30 mm, 5 μm; A: water (10 mM NH₄HCO₃); B: MeOH, B %: 15%-15% over 6 minutes] to afford the title compound (250 mg, 17% yield) as colorless oil.

Step D. (3R,5S)-tert-butyl 5-hydroxy-3-(hydroxymethyl)azepane-1-carboxylate: To a mixture of Pd/C (100 mg, 10% purity) in MeOH (6 mL) was added (3R,5S)-tert-butyl 5-(benzyloxy)-3-(hydroxymethyl)azepane-1-carboxylate (280 mg, 1.0 equiv) in MeOH (2 mL) under N2. The suspension was degassed under vacuum and purged with H2 several times. The mixture was stirred under H₂ (15 psi) at 50° C. for 10 hours. The mixture was filtered and concentrated to afford the title compound (230 mg, crude) as yellow solid. Step E. (4S,6R)-6-(hydroxymethyl)azepan-4-ol: To a solution of tert-butyl (3R,5S)-5-hydroxy-3-(hydroxymethyl)azepane-1-carboxylate (230 mg, 1.0 equiv) in DCM (2 mL) was added HCl•dioxane (3 mL, 13 equiv) at 0° C. The mixture was stirred at 0° C. for 30 minutes. The mixture was filtered and concentrated to give a residue. To the mixture in MeOH (3 mL) was added NaHCO₃ (30.0 mg). The mixture was stirred for 15 minutes. The mixture was filtered and concentrated to afford the title compound (130 mg, crude) as yellow oil.

The last two steps were performed according to Example 248. The title compound was obtained as white solid (FA salt). ¹H NMR (400 MHz, METHANOL-d4) δ=7.51 (dd, J=5.9, 8.9 Hz, 1H), 7.14 (t, J=9.2 Hz, 1H), 7.05-6.93 (m, 2H), 5.53-5.29 (m, 1H), 4.42-4.25 (m, 2H), 4.20-3.98 (m, 2H), 3.90-3.72 (m, 3H), 3.69-3.48 (m, 7H), 3.46-3.32 (m, 4H), 3.25-3.09 (m, 2H), 2.88-2.68 (m, 1H), 2.57-2.33 (m, 2H), 2.33-2.07 (m, 5H), 2.07-1.86 (m, 3H), 1.59-1.43 (m, 1H), 1.23-1.03 (m, 3H); LCMS (ESI, M+1): m/z=624.5.

### Example 370

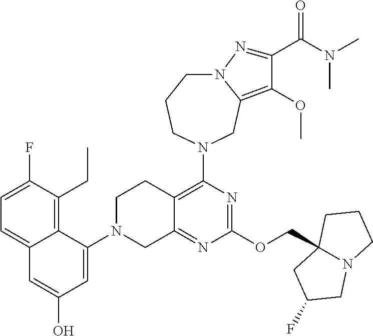 [SMILES: CCc1c(F)ccc2cc(O)cc(N3CCc4c(nc(OC[C@@]56CCCN5C[C@H](F)C6)nc4N4CCCn5nc(C(=O)N(C)C)c(OC)c5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-3-methoxy-N,N-dimethyl-5,6,7,8-tetrahydro-4H-pyrazolo[1,5-a][1,4]diazepine-2-carboxamide

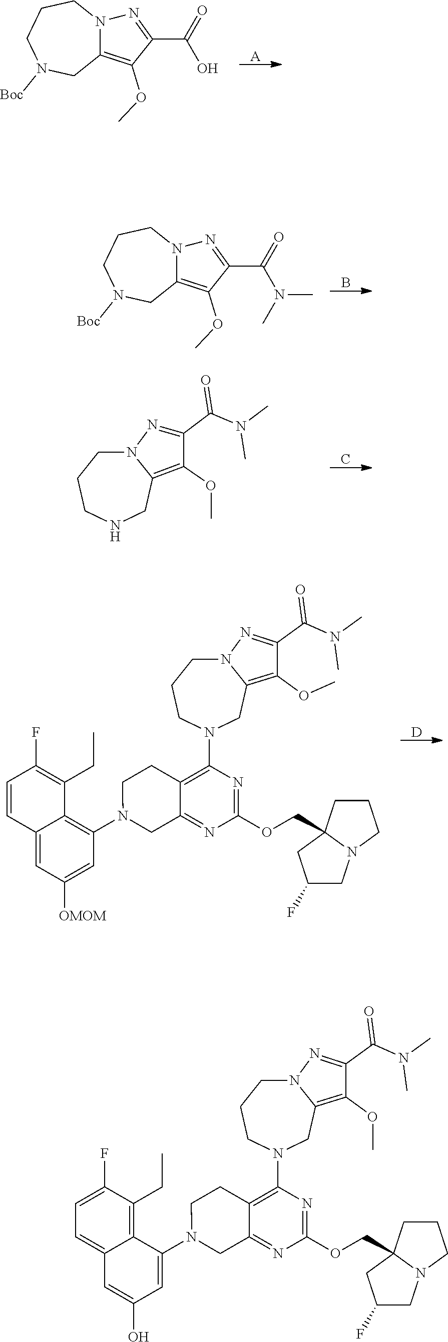

Synthesized according to Example 320 step G to J. The title compound was obtained as white solid (FA salt); 1H NMR (400 MHz, METHANOL-d4) δ=7.51 (dd, J=5.6, 8.8 Hz, 1H), 7.15 (t, J=9.2 Hz, 1H), 6.97 (br d, J=2.4 Hz, 2H), 5.52-5.30 (m, 1H), 4.90-4.87 (m, 2H), 4.52-4.37 (m, 2H), 4.36-4.26 (m, 2H), 4.20-4.02 (m, 3H), 3.77 (s, 3H), 3.68 (br d, J=18.0 Hz, 1H), 3.63-3.45 (m, 4H), 3.43-3.35 (m, 2H), 3.29-3.16 (m, 3H), 3.15 (s, 3H), 3.09 (s, 3H), 2.74 (br d, J=14.4 Hz, 1H), 2.54-2.32 (m, 2H), 2.32-2.20 (m, 2H), 2.19-1.98 (m, 4H), 1.11 (dt, J=2.4, 7.2 Hz, 3H); LCMS (ESI, M+1): m/z=717.5.

### Example 371

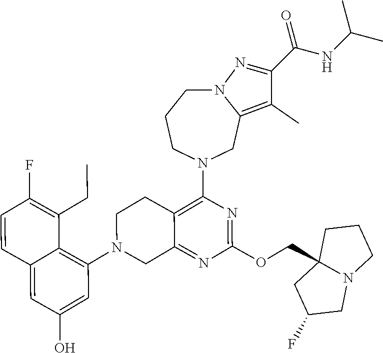 [SMILES: CCc1c(F)ccc2cc(O)cc(N3CCc4c(nc(OC[C@@]56CCCN5C[C@H](F)C6)nc4N4CCCn5nc(C(=O)NC(C)C)c(C)c5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N-isopropyl-3-methyl-5,6,7,8-tetrahydro-4H-pyrazolo[1,5-a][1,4]diazepine-2-carboxamide

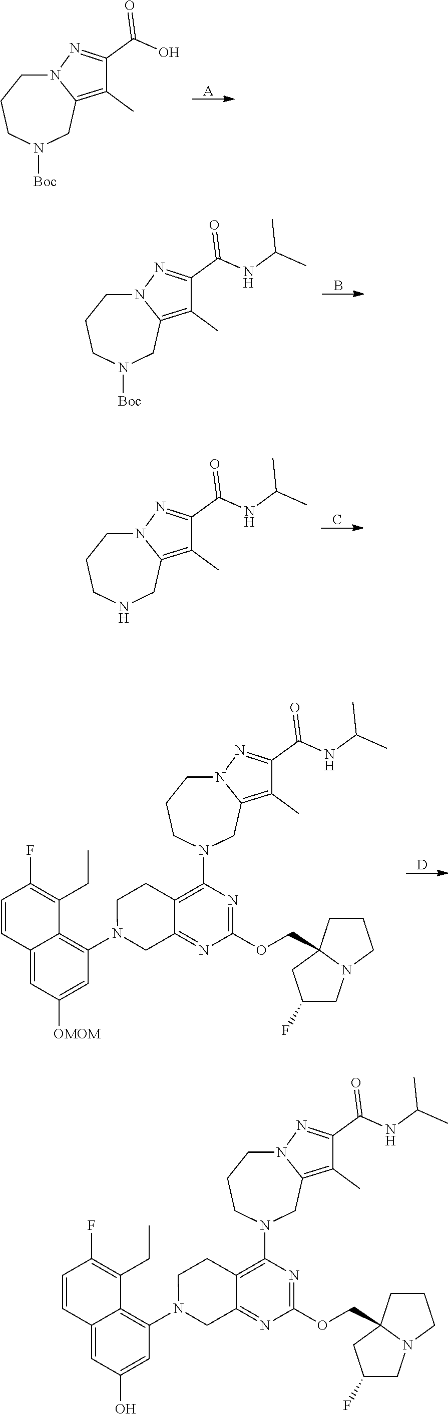

Step A. tert-butyl 2-(isopropylcarbamoyl)-3-methyl-7,8-dihydro-4H-pyrazolo[1,5-a][1,4]diazepine-5(6H)-carboxylate: To a mixture of 5-(tert-butoxycarbonyl)-3-methyl-5,6,7,8-tetrahydro-4H-pyrazolo[1,5-a][1,4]diazepine-2-carboxylic acid (100 mg, 1.0 equiv) and propan-2-amine (40.0 mg, 2.0 equiv) in DMF (2.5 mL) were added N,N-diethylpropan-2-amine (131 mg, 3.0 equiv) and HATU (258 mg, 2.0 equiv). The reaction was stirred at 20° C. for 1 hour. The mixture was diluted with H₂O (30 mL) and extracted with EtOAc (3×20 mL). The combined organic layers were dried over anhydrous sodium sulfate, concentrated, and purified by reversed phase chromatography [C18, 0.1% formic acid condition] to afford the title compound (110 mg, 94% yield) as a yellow soild; ¹H NMR (400 MHz, dimethylsulfoxide-d6) δ=7.61-7.45 (m, 1H), 4.50-4.27 (m, 4H), 4.08-3.96 (m, 1H), 3.65 (br s, 2H), 2.21-2.16 (m, 3H), 1.75 (br s, 2H), 1.36 (s, 9H), 1.12 (d, J=6.4 Hz, 6H).

Step B. N-isopropyl-3-methyl-5,6,7,8-tetrahydro-4H-pyrazolo[1,5-a][1,4]diazepine-2-carboxamide: To a mixture of tert-butyl 2-(isopropylcarbamoyl)-3-methyl-7,8-dihydro-4H-pyrazolo[1,5-a][1,4]diazepine-5(6H)-carboxylate (110 mg, 1.0 equiv) in ACN (0.61 mL) was added HCl•dioxane (4 M, 1.23 mL, 15 equiv). The reaction was stirred at 20° C. for 0.5 hours. The mixture was concentrated to afford the title compound (85 mg, crude HCl) as yellow solid.

The last two steps were performed according to Example 248. The title compound was obtained as off-white solid (FA salt). ¹H NMR (400 MHz, methanol-d4) δ=7.54-7.45 (m, 1H), 7.18-7.10 (m, 1H), 7.01-6.90 (m, 2H), 5.40-5.20 (m, 1H), 4.96-4.88 (m, 1H), 4.73-4.67 (m, 1H), 4.53-4.45 (m, 1H), 4.44-4.35 (m, 1H), 4.17-4.00 (m, 5H), 3.99-3.89 (m, 1H), 3.69-3.61 (m, 1H), 3.54-3.46 (m, 1H), 3.45-3.36 (m, 2H), 3.27-3.23 (m, 3H), 3.22-3.10 (m, 2H), 3.08-3.00 (m, 1H), 2.75-2.66 (m, 1H), 2.36-2.26 (m, 4H), 2.24-2.04 (m, 4H), 2.03-1.94 (m, 2H), 1.93-1.81 (m, 1H), 1.31-1.15 (m, 6H), 1.14-1.06 (m, 3H); ¹⁹F NMR (400 MHz, methanol-d4) δ=−123.030, -173.585; LCMS (ESI, M+1): m/z=715.3.

### Example 372

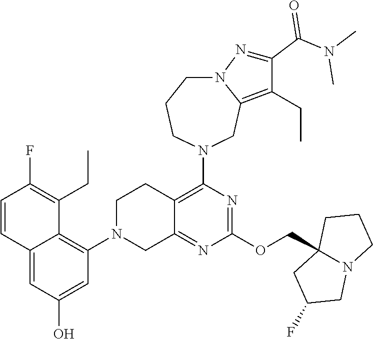 [SMILES: CCc1c(C(=O)N(C)C)nn2c1CN(c1nc(OC[C@@]34CCCN3C[C@H](F)C4)nc3c1CCN(c1cc(O)cc4ccc(F)c(CC)c14)C3)CCC2]

### 3-ethyl-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide

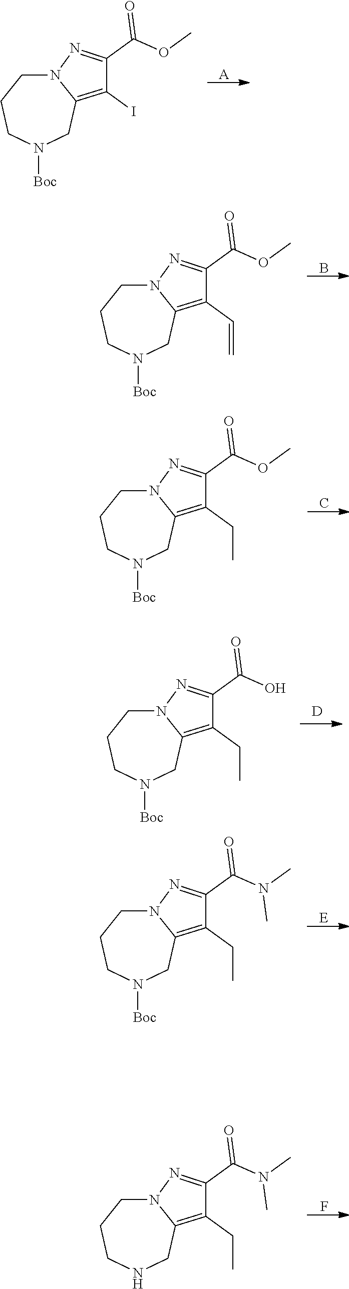

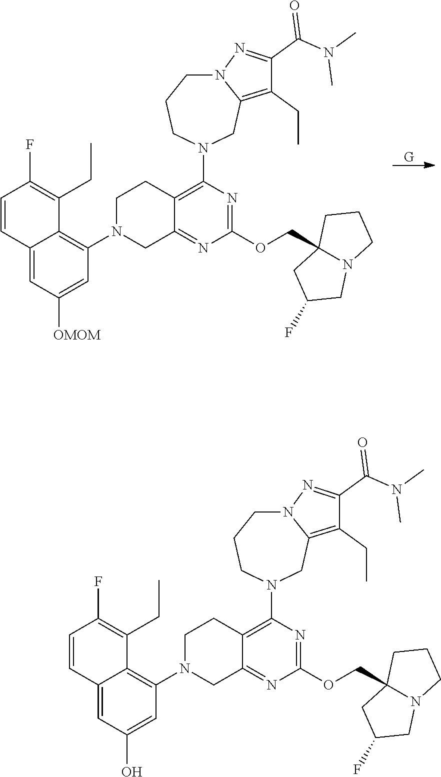

Step A. 5-tert-butyl 2-methyl 3-vinyl-7,8-dihydro-4H-pyrazolo[1,5-a][1,4]diazepine-2,5(6H)-dicarboxylate: A mixture of 5-tert-butyl 2-methyl 3-iodo-7,8-dihydro-4H-pyrazolo[1,5-a][1,4]diazepine-2,5(6H)-dicarboxylate (5.90 g, 1.0 equiv), 4,4,5,5-tetramethyl-2-vinyl-1,3,2-dioxaborolane (4.31 g, 2.0 equiv), [2-(2-aminophenyl)phenyl]palladium(1+);bis(1-adamantyl)-butyl-phosphane;methanesulfonate (1.02 g, 0.10 equiv), and NaHCO₃ (3.53 g, 3.0 equiv) in dioxane (70 mL) and H₂O (15 mL) was degassed and purged with nitrogen 3 times. The reaction was stirred at 80° C. for 2 hours under N2 atmosphere. The reaction was concentrated under vacuum to give a residue. The residue was diluted with H₂O (30 mL) and extracted with EtOAc (3×30 mL). The combined organic layers were washed with brine (30 mL), dried over anhydrous sodium sulfate, concentrated, and purified by reversed phase flash chromatography [C18, 0.1% formic acid condition] to afford the title compound (2.50 g, 55% yield) as yellow solid. LCMS (ESI, M+1): m/z=322.0.

Step B. 5-tert-butyl 2-methyl 3-ethyl-7,8-dihydro-4H-pyrazolo[1,5-a][1,4]diazepine-2,5(6H)-dicarboxylate: To a mixture of 5-tert-butyl 2-methyl 3-vinyl-7,8-dihydro-4H-pyrazolo[1,5-a][1,4]diazepine-2,5(6H)-dicarboxylate (2.50 g, 1.0 equiv) in MeOH (2 mL) was added Pd/C (400 mg, 10% purity). The mixture was degassed and purged with N2 3 times. The reaction was stirred at 25° C. for 1 hour under H2 at 15 psi. The mixture was filtered and concentrated under vacuum to afford the title compound (2.00 g, crude) as yellow solid.

Step C. 5-(tert-butoxycarbonyl)-3-ethyl-5,6,7,8-tetrahydro-4H-pyrazolo[1,5-a][1,4]diazepine-2-carboxylic acid: To a mixture of 5-tert-butyl 2-methyl 3-ethyl-7,8-dihydro-4H-pyrazolo[1,5-a][1,4]diazepine-2,5(6H)-dicarboxylate (2.90 g, 1.0 equiv) in THF (7 mL) and H₂O (7 mL) was added NaOH (5.38 g, 15 equiv). The reaction was stirred at 25° C. for 12 hours. The mixture was diluted with water (50 mL) and concentrated under vacuum to give a residue. The pH of the residue was adjusted to 4 with HCl (2 M). The residue was extracted with EtOAc (3×30 mL). The combined organic layers were dried over anhydrous sodium sulfate, concentrated, and purified by reversed phase flash chromatography [C18, 0.1% formic acid condition] to afford the title compound (3.50 g, 74% yield) as yellow oil.

Step D. tert-butyl 2-(dimethylcarbamoyl)-3-ethyl-7,8-dihydro-4H-pyrazolo[1,5-a][1,4]diazepine-5(6H)-carboxylate: To a mixture of 5-(tert-butoxycarbonyl)-3-ethyl-5,6,7,8-tetrahydro-4H-pyrazolo[1,5-a][1,4]diazepine-2-carboxylic acid (2.40 g, 1.0 equiv) and dimethylamine (3.50 g, 10 equiv) in DMF (10 mL) were added N,N-diethylpropan-2-amine (3.00 g, 3.0 equiv) and HATU (5.90 g, 2.0 equiv). The reaction was stirred at 20° C. for 2 hours. The mixture was filtered and purified by reversed phase flash chromatography [C18, 0.1% formic acid condition] to afford the title compound (1.60 g, 60% yield) as yellow oil. LCMS (ESI, M+1): m/z =337.5.

Step E. 3-ethyl-N,N-dimethyl-5,6,7,8-tetrahydro-4H-pyrazolo[1,5-a][1,4]diazepine-2-carboxamide: To a solution of tert-butyl 2-(dimethylcarbamoyl)-3-ethyl-7,8-dihydro-4H-pyrazolo[1,5-a][1,4]diazepine-5(6H)-carboxylate (4.70 g, 1.0 equiv) in MeCN (10 mL) was added HCl•dioxane (4 M, 5 mL). The reaction was stirred at 25° C. for 0.5 hours. The mixture was concentrated under vacuum to give a residue. The residue was diluted with MeOH (20 mL), Na₂CO₃ (10 g) was added, and the mixture was stirred for 0.5 hours. The mixture was filtered and concentrated under vacuum to afford the title compound (3.00 g, crude, HCl) as yellow oil.

The last two steps were performed according to Example 248. The title compound was obtained as off-white solid. ¹H NMR (400 MHz, METHANOL-d4) δ=7.50 (dd, J=5.6, 8.8 Hz, 1H), 7.14 (t, J=9.2 Hz, 1H), 7.02-6.89 (m, 2H), 5.35-5.16 (m, 1H), 5.11 (br dd, J=4.0, 16.0 Hz, 1H), 4.66 (br d, J=16.0 Hz, 1H), 4.54-4.38 (m, 2H), 4.16-3.97 (m, 4H), 3.94-3.80 (m, 1H), 3.67 (br d, J=17.6 Hz, 1H), 3.52-3.37 (m, 3H), 3.24-3.12 (m, 5H), 3.09 (d, J=6.8 Hz, 6H), 2.98 (dt, J=5.6, 9.2 Hz, 1H), 2.71-2.64 (m, 1H), 2.59 (q, J=7.4 Hz, 2H), 2.32-2.10 (m, 4H), 2.09-2.01 (m, 1H), 2.00-1.89 (m, 2H), 1.89-1.77 (m, 1H), 1.15-1.01 (m, 6H). LCMS (ESI, M+1): m/z=715.5.

### Example 373

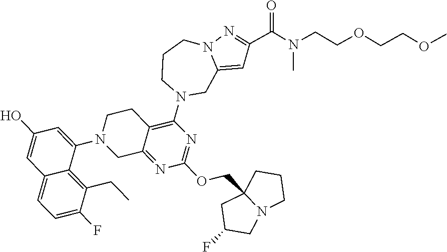 [SMILES: CCc1c(F)ccc2cc(O)cc(N3CCc4c(nc(OC[C@@]56CCCN5C[C@H](F)C6)nc4N4CCCn5nc(C(=O)N(C)CCOCCOC)cc5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N-(2-(2-methoxyethoxy)ethyl)-N-methyl-5,6,7,8-tetrahydro-4H-pyrazolo[1,5-a][1,4]diazepine-2-carboxamide

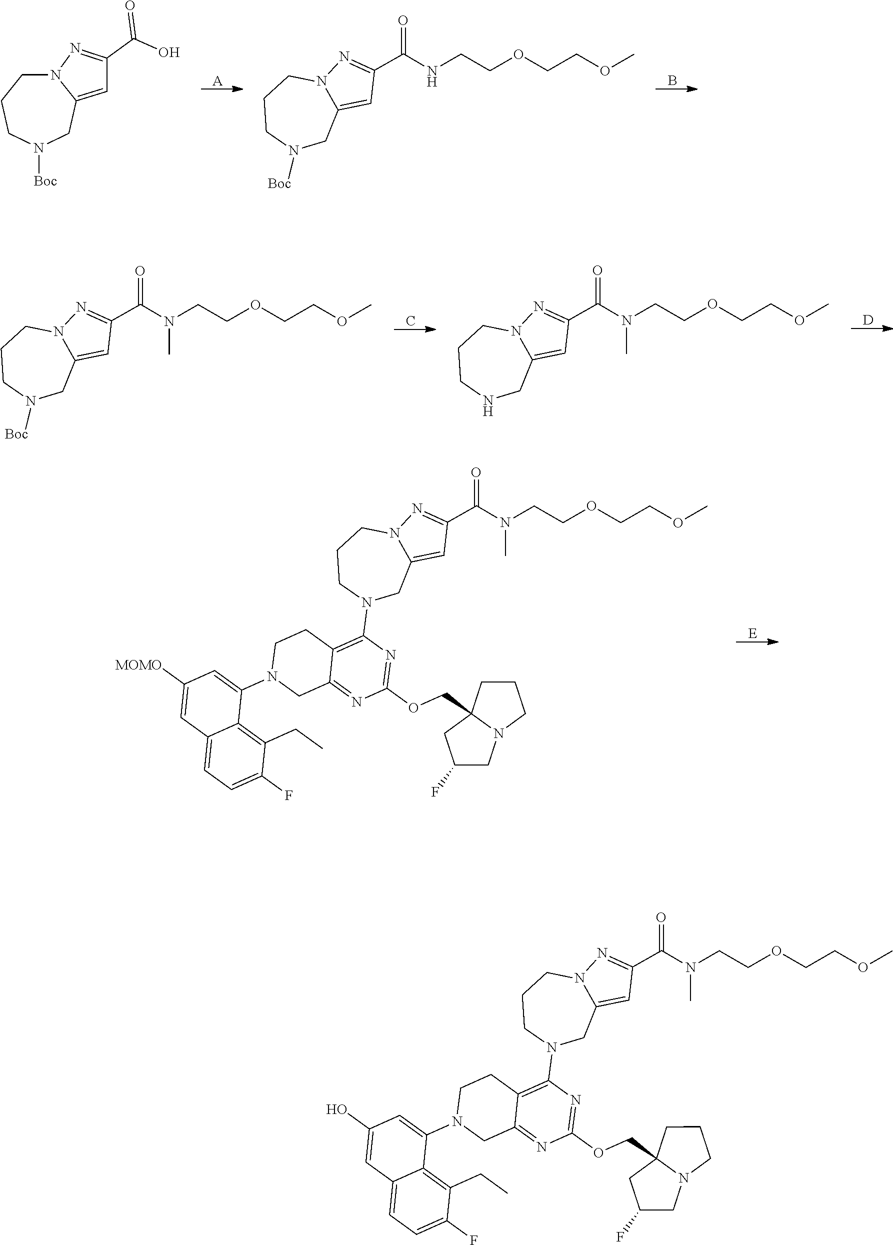

Step A. tert-butyl 2-((2-(2-methoxyethoxy)ethyl)carbamoyl)-7,8-dihydro-4H-pyrazolo[1,5-a][1,4]diazepine-5(6H)-carboxylate: To a mixture of 5-(tert-butoxycarbonyl)-5,6,7,8-tetrahydro-4H-pyrazolo[1,5-a][1,4]diazepine-2-carboxylic acid (150 mg, 1.0 equiv) and 2-(2-methoxyethoxy)ethanamine (127 mg, 2.0 equiv) in THF (2 mL) were added HATU (405 mg, 2.0 equiv) and TEA (162 mg, 3.0 equiv). The reaction was stirred at 25° C. for 2 hours. The mixture was concentrated and purified by reversed phase flash chromatography [C18, water (FA, 0.10%)/acetonitrile] to afford the title compound (160 mg, 71% yield) as yellow oil. LCMS (ESI, M+1): m/z=383.0.

Step B. tert-butyl 2-((2-(2-methoxyethoxy)ethyl)(methyl)carbamoyl)-7,8-dihydro-4H-pyrazolo[1,5-arl1,4]diazepine-5(6H)-carboxylate: To a mixture of tert-butyl 2-((2-(2-methoxyethoxy)ethyl)carbamoyl)-7,8-dihydro-4H-pyrazolo[1,5-a][1,4]diazepine-5(6H)-carboxylate (160 mg, 1.0 equiv) in THE (2 mL) was added NaH (53.3 mg, 3.2 equiv) while stirring at 0° C. The reaction was stirred at 0° C. for 0.5 hours. Then a solution of CH₃I (119 mg, 2.0 equiv) was added into the mixture at 0° C. The reaction was stirred at 25° C. for 2 hours. The mixture was diluted with water (10 mL) and extracted with EtOAc (3×10 mL). The combined organic layers were dried over anhydrous sodium sulfate and concentrated to afford the title compound (230 mg, crude) as yellow oil.

Step C. N-(2-(2-methoxyethoxy)ethyl)-N-methyl-5,6,7,8-tetrahydro-4H-pyrazolo[1,5-a][1,4]diazepine-2-carboxamide: To a mixture of tert-butyl 2-((2-(2-methoxyethoxy)ethyl)(methyl)carbamoyl)-7,8-dihydro-4H-pyrazolo[1,5-a][1,4]diazepine-5(6H)-carboxylate (220 mg, 1.0 equiv) in MeOH (1 mL) was added HCl•dioxane (0.5 mL, 4M). The reaction was stirred at 25° C. for 1 hour. The mixture was concentrated under vacuum to afford the title compound (230 mg, crude, HCl) as yellow oil.

The last two steps were performed according to Example 248. The title compound was obtained as off-white solid (FA salt). ¹H NMR (400 MHz, METHANOL-d4) δ=7.51 (dd, J=6.0, 8.4 Hz, 1H), 7.14 (t, J=9.2 Hz, 1H), 6.97 (s, 2H), 6.63 (br d, J=10.8 Hz, 1H), 5.50-5.27 (m, 1H), 5.04-4.85 (m, 2H), 4.60-4.45 (m, 2H), 4.33-4.13 (m, 3H), 4.06 (br d, J=17.2 Hz, 2H), 3.97 (br s, 1H), 3.78-3.59 (m, 5H), 3.57-3.43 (m, 7H), 3.36 (br d, J=18.0 Hz, 5H), 3.29-3.15 (m, 4H), 3.11 (s, 2H), 2.74 (br d, J=14.0 Hz, 1H), 2.51-2.36 (m, 1H), 2.36-2.19 (m, 3H), 2.18-2.03 (m, 3H), 2.03-1.93 (m, 1H), 1.10 (br t, J=6.4 Hz, 3H). LCMS (ESI, M+1): m/z=775.5.

### Example 374

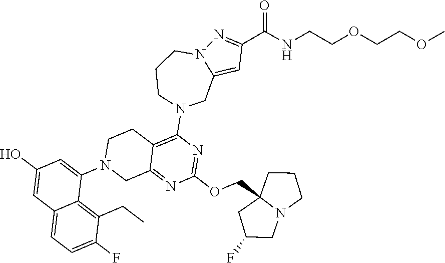 [SMILES: CCc1c(F)ccc2cc(O)cc(N3CCc4c(nc(OC[C@@]56CCCN5C[C@H](F)C6)nc4N4CCCn5nc(C(=O)NCCOCCOC)cc5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N-(2-(2-methoxyethoxy)ethyl)-5,6,7,8-tetrahydro-4H-pyrazolo[1,5-a][1,4]diazepine-2-carboxamide

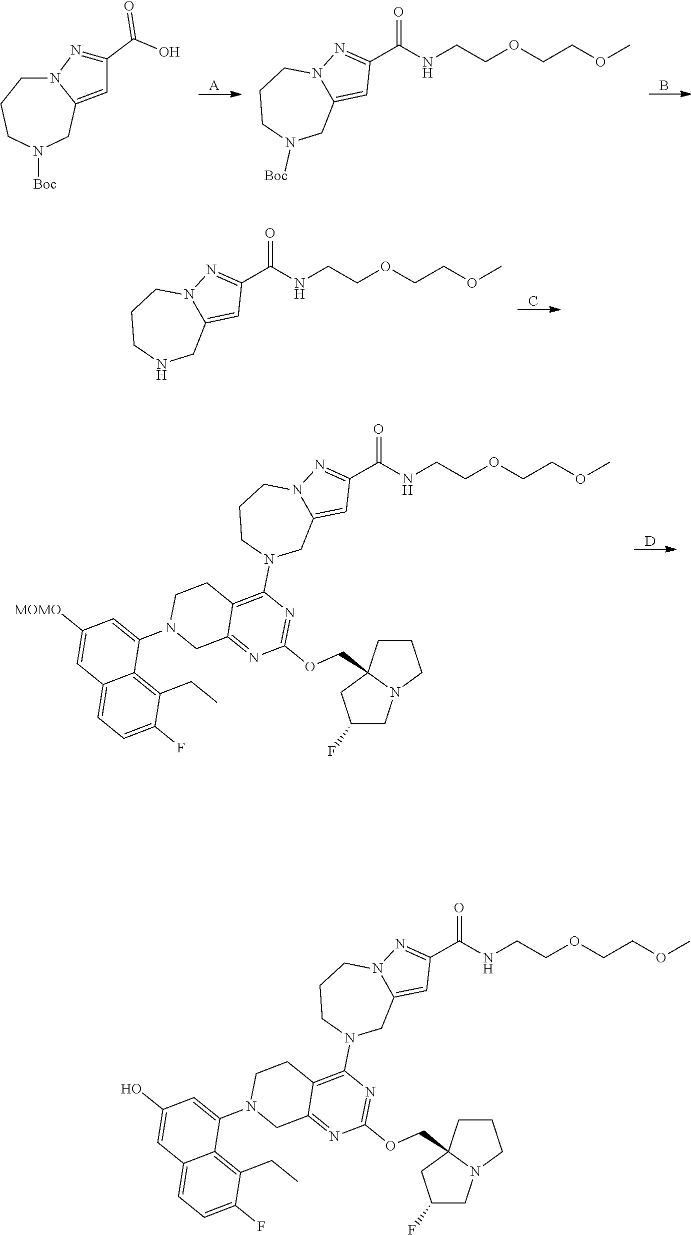

Step A. tert-butyl 2-((2-(2-methoxyethoxy)ethyl)carbamoyl)-7,8-dihydro-4H-pyrazolo[1,5-a][1,4]diazepine-5(6H)-carboxylate: To a mixture of 5-(tert-butoxycarbonyl)-5,6,7,8-tetrahydro-4H-pyrazolo[1,5-a][1,4]diazepine-2-carboxylic acid (150 mg, 1.0 equiv), and 2-(2-methoxyethoxy)ethanamine (127 mg, 2.0 equiv) in THF (2 mL) was added HATU (405 mg, 2.0 equiv) and TEA (162 mg, 3.0 equiv). The reaction was stirred at 25° C. for 2 hours. The mixture was concentrated and purified by reversed phase flash chromatography [C18, water (FA, 0.10%)/acetonitrile] to afford the title compound (150 mg, 70% yield) as yellow oil. LCMS (ESI, M+1): m/z=383.0.

Step B. N-(2-(2-methoxyethoxy)ethyl)-5,6,7,8-tetrahydro-4H-pyrazolo[1,5-a][1,4]diazepine-2-carboxamide: To a mixture of tert-butyl 2-((2-(2-methoxyethoxy)ethyl)carbamoyl)-7,8-dihydro-4H-pyrazolo[1,5-a][1,4]diazepine-5(6H)-carboxylate (150 mg, 1.0 equiv) in MeOH (0.5 mL) was added HCl•dioxane (4 M, 1 mL). The reaction was with stirred at 25° C. for 1 hour. The mixture was concentrated under vacuum to afford the title compound (180 mg, crude, HCl) as yellow oil.

The last two steps were performed according to Example 248. The title compound was obtained as off-white solid (FA salt). ¹H NMR (400 MHz, METHANOL-d4) δ=7.51 (dd, J=6.0, 8.8 Hz, 1H), 7.14 (t, J=9.2 Hz, 1H), 6.97 (s, 2H), 6.71 (s, 1H), 5.51-5.31 (m, 1H), 5.01-4.86 (m, 2H), 4.61-4.46 (m, 2H), 4.35-4.14 (m, 3H), 4.11-3.98 (m, 2H), 3.69 (s, 1H), 3.64-3.61 (m, 3H), 3.61 (br s, 1H), 3.57-3.53 (m, 5H), 3.49 (br s, 2H), 3.38 (br d, J=6.8 Hz, 2H), 3.35 (s, 3H), 3.28-3.13 (m, 4H), 2.74 (br d, J=13.6 Hz, 1H), 2.54-2.38 (m, 1H), 2.38-2.21 (m, 3H), 2.19-2.09 (m, 2H), 2.08-1.90 (m, 2H), 1.11 (br t, J=7.2 Hz, 3H). LCMS (ESI, M+1): m/z=761.4.

### Example 375

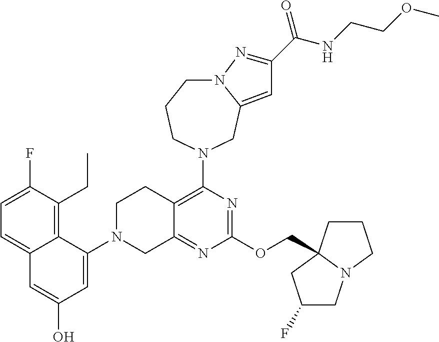 [SMILES: CCc1c(F)ccc2cc(O)cc(N3CCc4c(nc(OC[C@@]56CCCN5C[C@H](F)C6)nc4N4CCCn5nc(C(=O)NCCOC)cc5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N-(2-methoxyethyl)-5,6,7,8-tetrahydro-4H-pyrazolo[1,5-a][1,4]diazepine-2-carboxamide

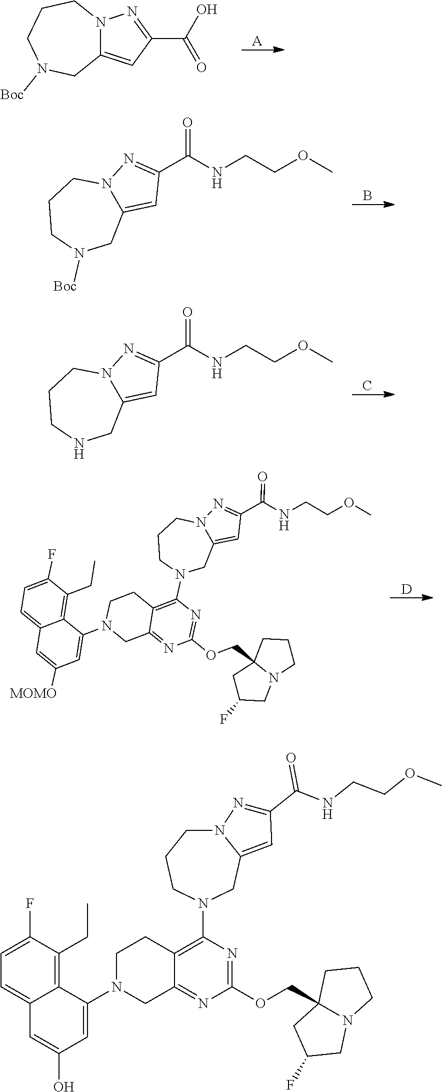

Step A. tert-butyl 2-((2-methoxyethyl)carbamoyl)-7,8-dihydro-4H-pyrazolo[1,5-a][1,4]diazepine-5(6H)-carboxylate: To a mixture of 5-tert-butoxycarbonyl-4,6,7,8-tetrahydropyrazolo[1,5-a][1,4]diazepine-2-carboxylic acid (150 mg, 1.0 equiv) and 2-methoxyethanamine (80.1 mg, 2.0 equiv) in DCM (1 mL) were added HATU (304 mg, 1.5 equiv) and N,N-diethylpropan-2-amine (275 mg, 4.0 equiv). The reaction was stirred at 25° C. for 12 hours. The mixture was concentrated and purified by reversed phase flash chromatography [water (0.1%, FA)/acetonitrile] to afford the title compound (150 mg, 83% yield) as yellow solid; LCMS (ESI, M+1): m/z=339.0.

Step B. N-(2-methoxyethyl)-5,6,7,8-tetrahydro-4H-pyrazolo[1,5-a][1,4]diazepine-2-carboxamide: A mixture of tert-butyl 2-(2-methoxyethylcarbamoyl)-4,6,7,8-tetrahydropyrazolo[1,5-a][1,4]diazepine-5-carboxylate (140 mg, 1.0 equiv) in HCl/dioxane (4 M, 68 equiv) and MeCN (1 mL) was stirred at 20° C. for 0.5 hours. The mixture was concentrated under vacuum to afford the title compound (70 mg, crude) as yellow solid.

The last two steps were performed according to Example 248. The title compound was obtained as orange solid. ¹H NMR (400 MHz, METHANOL-d4) δ=7.52 (dd, J=5.6 8.8 Hz, 1H), 7.20-7.13 (m, 1H), 6.99 (s, 2H), 6.71 (s, 1H), 5.37-5.18 (m, 1H), 5.03-4.97 (m, 1H), 4.81 (br s, 1H), 4.58-4.49 (m, 2H), 4.26-4.17 (m, 1H), 4.12-3.99 (m, 4H), 3.67 (br d, J=16.0 Hz, 1H), 3.54 (s, 4H), 3.42 (br d, J=7.2 Hz, 2H), 3.39 (s, 4H), 3.27-3.20 (m, 3H), 3.20-3.13 (m, 2H), 3.04-2.95 (m, 1H), 2.78-2.70 (m, 1H), 2.41-2.20 (m, 2H), 2.02-1.82 (m, 7H), 1.13 (br t, J=7.2 Hz, 3H) LCMS (ESI, M+1): m/z=717.2

### Example 376

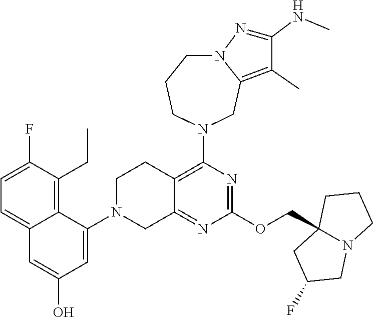 [SMILES: CCc1c(F)ccc2cc(O)cc(N3CCc4c(nc(OC[C@@]56CCCN5C[C@H](F)C6)nc4N4CCCn5nc(NC)c(C)c5C4)C3)c12]

### 5-ethyl-6-fluoro-4-(2-(((2R,7aS)-2-fluorohexahydro-1H-pyrrolizin-7a-yl)methoxy)-4-(3-methyl-2-(methylamino)-7,8-dihydro-4H-pyrazolo[1,5-a][1,4]diazepin-5(6H)-yl)-5,6-dihydropyrido[3,4-d]pyrimidin-7(8H)-yl)naphthalen-2-ol

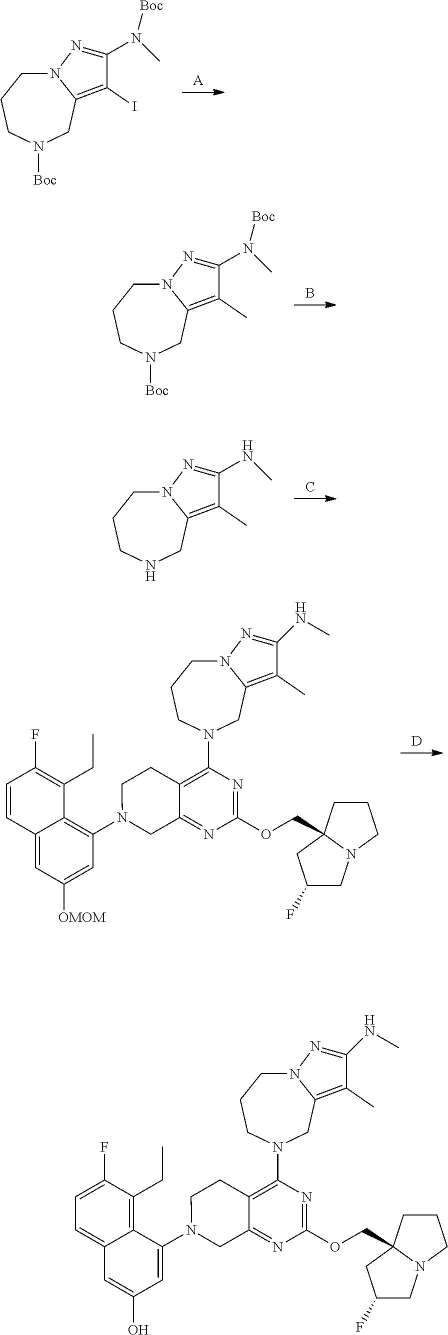

Step A. tert-butyl 2-((tert-butoxycarbonyl)(methyl)amino)-3-methyl-7,8-dihydro-4H-pyrazolo[1,5-a][1,4]diazepine-5(6H)-carboxylate: A mixture of tert-butyl 2-((tert-butoxycarbonyl)(methyl)amino)-3-iodo-7,8-dihydro-4H-pyrazolo[1,5-a][1,4]diazepine-5(6H)-carboxylate (150 mg, 1.0 equiv), 2,4,6-trimethyl-1,3,5,2,4,6-trioxatriborinane (3.5 M, 50% purity, 2.0 equiv), Cs2CO₃ (297 mg, 3.0 equiv), and Pd(dppf)C12 (11.1 mg, 0.05 equiv) in dioxane (6 mL) and H₂O (0.6 mL) was purged with N2 3 times and then the mixture was stirred at 90° C. for 1 hour. The mixture was quenched with water (10 mL) and extracted with ethyl acetate (2×8 mL). The combined organic layers were dried over anhydrous sodium sulfate, concentrated, and purified by prep-HPLC (column: Phenomenex luna C18 150×25 mm×10 m; A: water (FA); B: ACN, B %: 46%-76%, 10 min) to afford the title compound (60.0 mg, 48% yield) as white solid. LCMS (ESI, M+1): m/z=381.3.

Step B. N,3-dimethyl-5,6,7,8-tetrahydro-4H-pyrazolo[1,5-a][1,4]diazepin-2-amine: To a solution of tert-butyl 2-((tert-butoxycarbonyl)(methyl)amino)-3-methyl-7,8-dihydro-4H-pyrazolo[1,5-a][1,4]diazepine-5(6H)-carboxylate (50.0 mg, 1.0 equiv) in acetonitrile (2 mL) was added HCl•dioxane (4 M, 60 equiv). The mixture was stirred at 0° C. for 1 hour. The reaction was concentrated under reduced pressure to dryness. Methanol (3 mL) was added and the pH was adjusted to 8 with NaHCO₃solid. The mixture was filtered. The filtrate was concentrated under reduced pressure to dryness. The residue was diluted with dichloromethane/methanol (10/1, 3 mL) and filtered. The filtrate was concentrated under reduced pressure to afford the title compound (20.0 mg, 82% yield) as yellow oil. LCMS (ESI, M+1): m/z=181.2.

The last two steps were performed according to Example 248. The title compound was obtained as white solid. ¹H NMR (400 MHz, METHANOL-d4) δ=7.52 (br d, J=3.2 Hz, 1H), 7.15 (t, J=9.2 Hz, 1H), 6.97 (br d, J=3.6 Hz, 2H), 5.51-5.31 (m, 1H), 4.70-4.54 (m, 1H), 4.32-4.16 (m, 4H), 4.12-3.96 (m, 2H), 3.96-3.84 (m, 1H), 3.72-3.60 (m, 1H), 3.52-3.44 (m, 3H), 3.44-3.36 (m, 2H), 3.24-3.20 (m, 2H), 3.20-3.12 (m, 2H), 2.80 (s, 3H), 2.76-2.68 (m, 1H), 2.36-2.24 (m, 2H), 2.20-2.00 (m, 4H), 1.91 (s, 3H), 1.37 (d, J=6.8 Hz, 3H), 1.10 (t, J=7.2 Hz, 3H); LCMS (ESI, M+1): m/z=659.3.

### Example 377

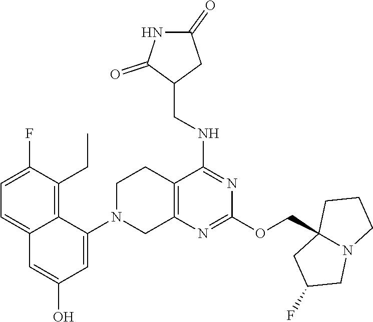 [SMILES: CCc1c(F)ccc2cc(O)cc(N3CCc4c(nc(OC[C@@]56CCCN5C[C@H](F)C6)nc4NCC4CC(=O)NC4=O)C3)c12]

### 5-(((7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)amino)methyl)imidazolidine-2,4-dione

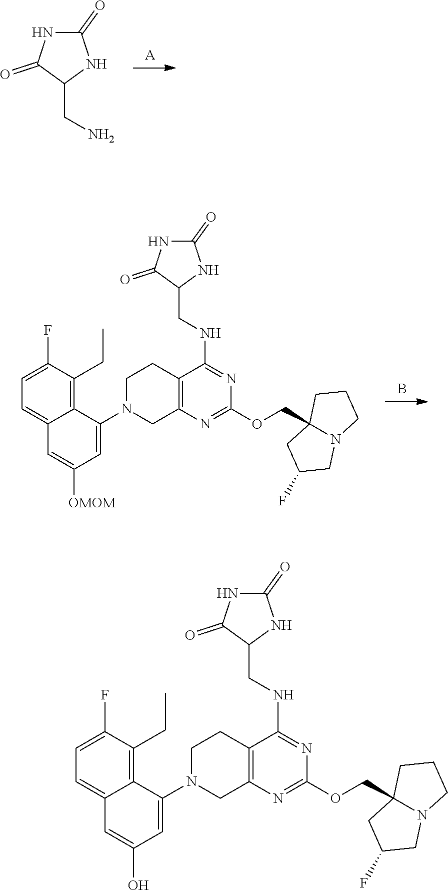

Synthesized according to Example 248. The title compound was obtained as orange solid (FA salt). 1H NMR (400 MHz, dimethylsulfoxide-d6+deuterium oxide-d2) δ=7.58 (dd, J=6.0, 9.2 Hz, 1H), 7.23 (s, 1H), 7.02-6.96 (m, 2H), 5.34-5.16 (m, 1H), 4.33 (dd, J=5.6, 8.8 Hz, 1H), 3.99-3.91 (m, 1H), 3.89-3.84 (m, 1H), 3.79-3.69 (m, 2H), 3.63-3.58 (m, 1H), 3.29-3.17 (m, 3H), 3.14-2.97 (m, 4H), 2.87-2.76 (m, 1H), 2.70-2.65 (m, 1H), 2.11-1.91 (m, 4H), 1.87-1.65 (m, 4H), 1.01-0.97 (m, 3H); 19F NMR (377 MHz, dimethylsulfoxide-d6+deuterium oxide-d2) 6=-121.356, -171.962; LCMS (ESI, M+1): m/z=608.3.

### Example 378

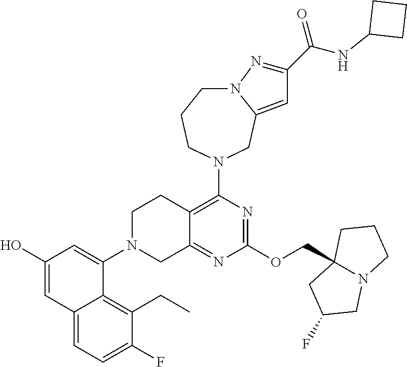 [SMILES: CCc1c(F)ccc2cc(O)cc(N3CCc4c(nc(OC[C@@]56CCCN5C[C@H](F)C6)nc4N4CCCn5nc(C(=O)NC6CCC6)cc5C4)C3)c12]

### N-cyclobutyl-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepine-2-carboxamide

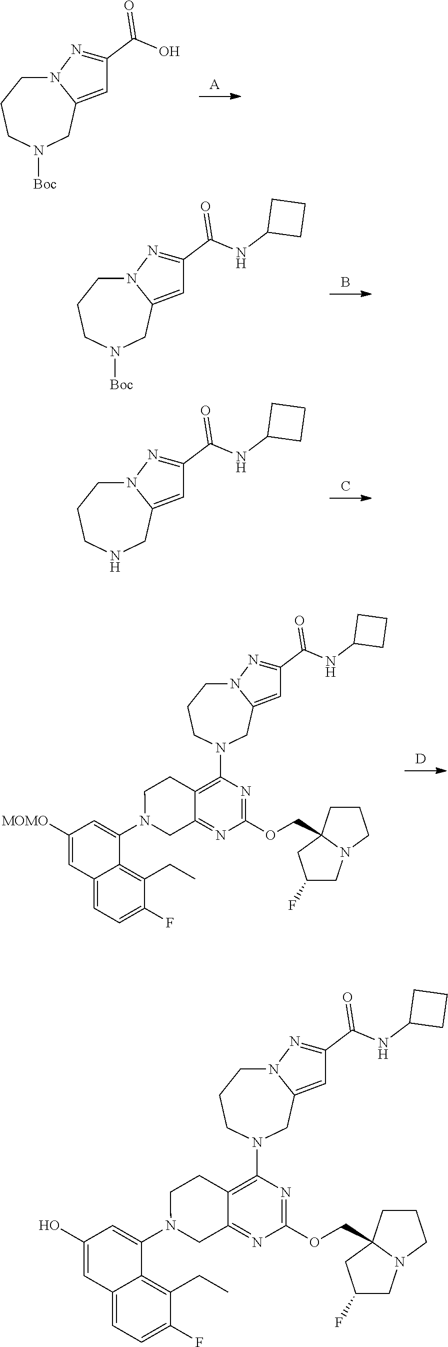

Step A. tert-butyl 2-(cyclobutylcarbamoyl)-7,8-dihydro-4H-pyrazolo[1,5-a][1,4]diazepine-5(6H)-carboxylate: To a mixture of 5-(tert-butoxycarbonyl)-5,6,7,8-tetrahydro-4H-pyrazolo[1,5-a][1,4]diazepine-2-carboxylic acid (150 mg, 1.0 equiv) and cyclobutanamine (75.8 mg, 2.0 equiv) in DCM (2 mL) were added HATU (304 mg, 1.5 equiv) and N,N-diethylpropan-2-amine (276 mg, 4.0 equiv). The reaction was stirred at 20° C. for 0.5 hours. The mixture was concentrated and purified with reversed phase flash chromatography [C18, 0.1% formic acid condition] to afford the title compound (75.0 mg, 41% yield) as a yellow solid; LCMS (ESI, M+1): m/z=335.1.

Step B. N-cyclobutyl-5,6,7,8-tetrahydro-4H-pyrazolo[1,5-a][1,4]diazepine-2-carboxamide: To a mixture of tert-butyl 2-(cyclobutylcarbamoyl)-7,8-dihydro-4H-pyrazolo[1,5-a][1,4]diazepine-5(6H)-carboxylate (80.0 mg, 1.0 equiv) in MeCN (1 mL) was add HCl•dioxane (4 M, 1.0 mL, 17 equiv). The reaction was stirred at 20° C. for 0.5 hours. The mixture was concentrated under vacuum to afford the title compound (49.0 mg, 69% yield) as a yellow solid; LCMS (ESI, M+1): m/z=235.1.

The last two steps were performed according to Example 248. The title compound was obtained as white solid (FA salt). ¹H NMR (400 MHz, METHANOL-d4) δ=7.53 (dd, J=5.6, 9.2 Hz, 1H), 7.16 (t, J=9.2 Hz, 1H), 7.03-6.96 (m, 2H), 6.71 (s, 1H), 5.42-5.24 (m, 1H), 5.02-4.94 (m, 1H), 4.83 (br d, J=2.0 Hz, 1H), 4.62-4.45 (m, 3H), 4.2-4.01 (m, 5H), 3.71-3.63 (m, 1H), 3.60-3.51 (m, 1H), 3.47-3.34 (m, 4H), 3.28-3.14 (m, 3H), 3.09 (dt, J=5.6, 9.6 Hz, 1H), 2.75 (br d, J=14.2 Hz, 1H), 2.43-2.18 (m, 5H), 2.17-1.93 (m, 6H), 1.92-1.71 (m, 3H), 1.13 (t, J=7.2 Hz, 3H). LCMS (ESI, M+1): m/z=713.4

### Example 379

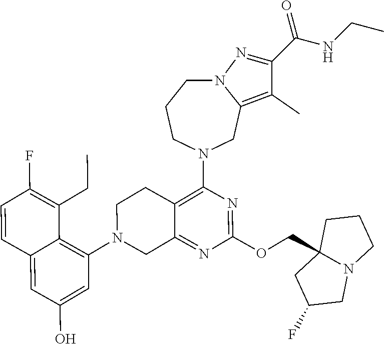 [SMILES: CCNC(=O)c1nn2c(c1C)CN(c1nc(OC[C@@]34CCCN3C[C@H](F)C4)nc3c1CCN(c1cc(O)cc4ccc(F)c(CC)c14)C3)CCC2]

### N-ethyl-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-3-methyl-5,6,7,8-tetrahydro-4H-pyrazolo[1,5-a][1,4]diazepine-2-carboxamide

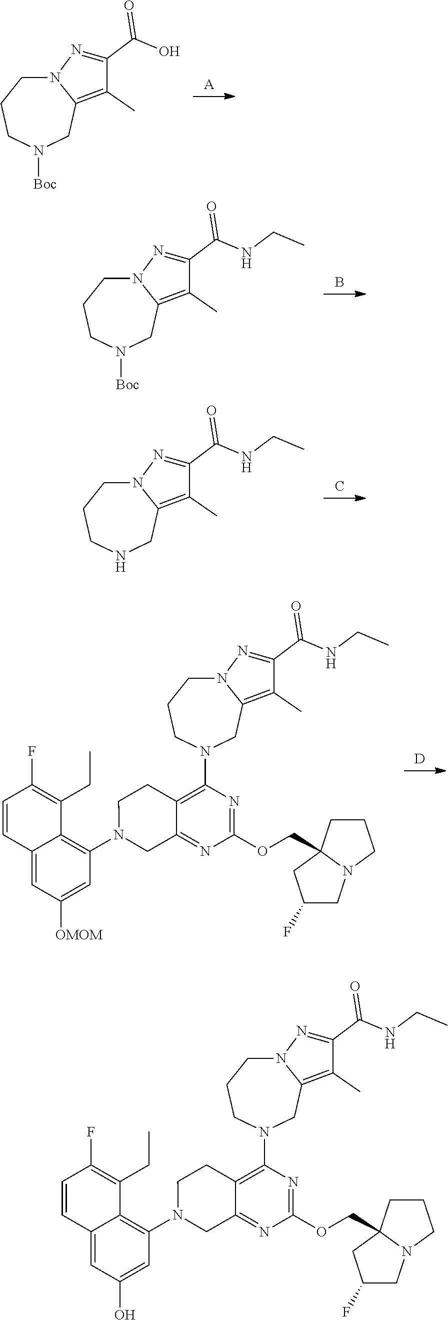

Step A. tert-butyl 2-(ethylcarbamoyl)-3-methyl-7,8-dihydro-4H-pyrazolo[1,5-a]l[1,4]diazepine-5(6H)-carboxylate: To a mixture of ethanamine (2 M, 271 μL, 2.0 equiv) and 5-(tert-butoxycarbonyl)-3-methyl-5,6,7,8-tetrahydro-4H-pyrazolo[1,5-a][1,4]diazepine-2-carboxylic acid (80.0 mg, 1.0 equiv) in DMF (2 mL) were added HATU (206 mg, 2.0 equiv) and N,N-diethylpropan-2-amine (105 mg, 3.0 equiv). The reaction was stirred at 25° C. for 1 hour. The mixture was diluted with H₂O (20 mL) and extracted with EtOAc (3×20 mL). The combined organic layers were dried over anhydrous sodium sulfate, concentrated, and purified by reversed phase flash chromatography [C18, 0.1% formic acid condition] to afford the title compound (85.0 mg, 97% yield) as brown solid; ¹H NMR (400 MHz, dimethylsulfoxide-d6) δ=7.89 (br d, J=5.2 Hz, 1H), 4.44-4.35 (m, 4H), 3.65 (br s, 2H), 3.25-3.15 (m, 2H), 2.19 (s, 3H), 1.75 (br s, 2H), 1.36 (s, 9H), 1.06 (t, J=7.2 Hz, 3H).

Step B. N-ethyl-3-methyl-5,6,7,8-tetrahydro-4H-pyrazolo[1,5-a][1,4]diazepine-2-carboxamide: To a mixture of tert-butyl 2-(ethylcarbamoyl)-3-methyl-7,8-dihydro-4H-pyrazolo[1,5-a][1,4]diazepine-5(6H)-carboxylate (90.0 mg, 1.0 equiv) in ACN (0.5 mL) was added HCl•dioxane (4 M, 1.05 mL, 15 equiv). The reaction was stirred at 20° C. for 0.5 hours. The mixture was concentrated to afford the title compound (70 mg, crude, HCl) as yellow solid.

The last two steps were performed according to Example 248. The title compound was obtained as orange solid (FA salt). ¹H NMR (400 MHz, methanol-d4) δ=7.54-7.47 (m, 1H), 7.18-7.11 (m, 1H), 6.99-6.91 (m, 2H), 5.42-5.22 (m, 1H), 4.96-4.88 (m, 1H), 4.74-4.65 (m, 1H), 4.54-4.35 (m, 2H), 4.14-4.02 (m, 4H), 4.01-3.90 (m, 1H), 3.69-3.61 (m, 1H), 3.54-3.46 (m, 1H), 3.46-3.32 (m, 6H), 3.27-3.02 (m, 4H), 2.75-2.66 (m, 1H), 2.40-2.29 (m, 4H), 2.29-2.19 (m, 2H), 2.18-1.98 (m, 4H), 1.96-1.81 (m, 1H), 1.22-1.15 (m, 3H), 1.13-1.06 (m, 3H); ¹⁹F NMR (400 MHz, methanol-d4) δ=−123.052, -173.651; LCMS (ESI, M+1): m/z=701.4.

### Example 380

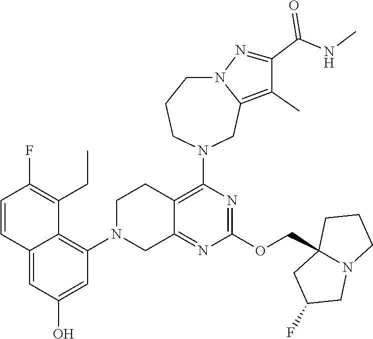 [SMILES: CCc1c(F)ccc2cc(O)cc(N3CCc4c(nc(OC[C@@]56CCCN5C[C@H](F)C6)nc4N4CCCn5nc(C(=O)NC)c(C)c5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,3-dimethyl-5,6,7,8-tetrahydro-4H-pyrazolo[1,5-a][1,4]diazepine-2-carboxamide

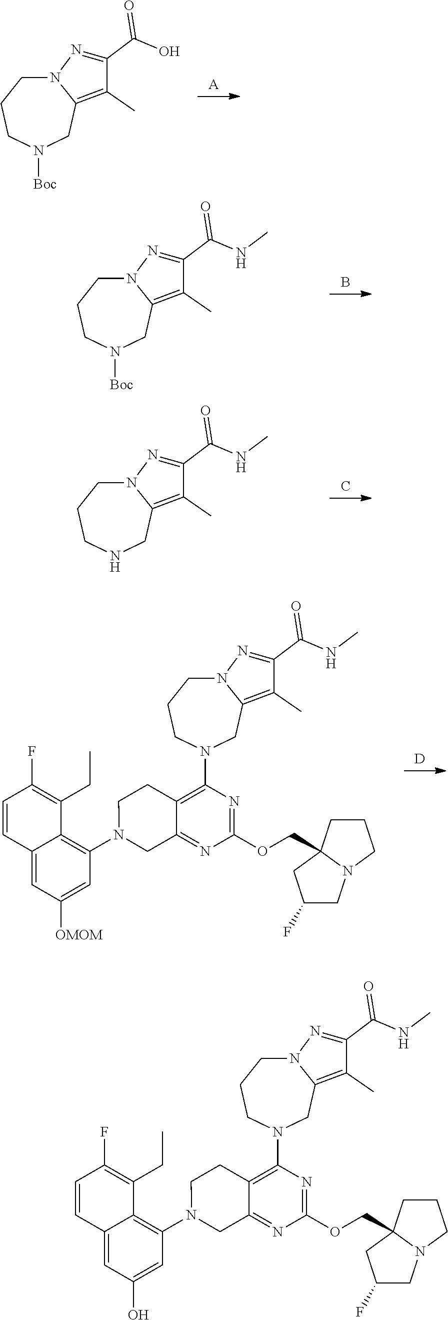

Synthesized according to Example 379. The title compound was obtained as orange solid (FA salt). ¹H NMR (400 MHz, methanol-d₄) δ=7.54-7.48 (m, 1H), 7.18-7.10 (m, 1H), 6.99-6.93 (m, 2H), 5.44-5.25 (m, 1H), 4.96-4.88 (m, 1H), 4.74-4.67 (m, 1H), 4.53-4.45 (m, 1H), 4.44-4.35 (m, 1H), 4.19-4.10 (m, 2H), 4.09-4.04 (m, 2H), 4.00-3.91 (m, 1H), 3.69-3.61 (m, 1H), 3.54-3.47 (m, 1H), 3.46-3.35 (m, 4H), 3.14 (br dd, J=10.8, 18.8 Hz, 4H), 2.82 (br s, 3H), 2.74-2.67 (m, 1H), 2.41-2.29 (m, 4H), 2.29-2.18 (m, 2H), 2.16-2.00 (m, 4H), 1.96-1.82 (m, 1H), 1.14-1.05 (m, 3H); ¹⁹F NMR (400 MHz, methanol-d4) δ=−123.044, -173.688; LCMS (ESI, M+1): m/z=687.6.

### Example 381

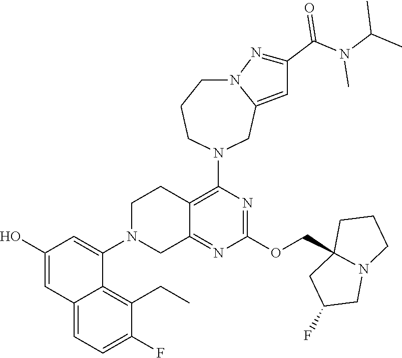 [SMILES: CCc1c(F)ccc2cc(O)cc(N3CCc4c(nc(OC[C@@]56CCCN5C[C@H](F)C6)nc4N4CCCn5nc(C(=O)N(C)C(C)C)cc5C4)C3)c12]

### 5-(7-(8-ethyl-7-fluoro-3-hydroxynaphthalen-1-yl)-2-(((2R,7aS)-2-fluorohexahydro-11H-pyrrolizin-7a-yl)methoxy)-5,6,7,8-tetrahydropyrido[3,4-d]pyrimidin-4-yl)-N-isopropyl-N-methyl-5,6,7,8-tetrahydro-4H-pyrazolo[1,5-a][1,4]diazepine-2-carboxamide

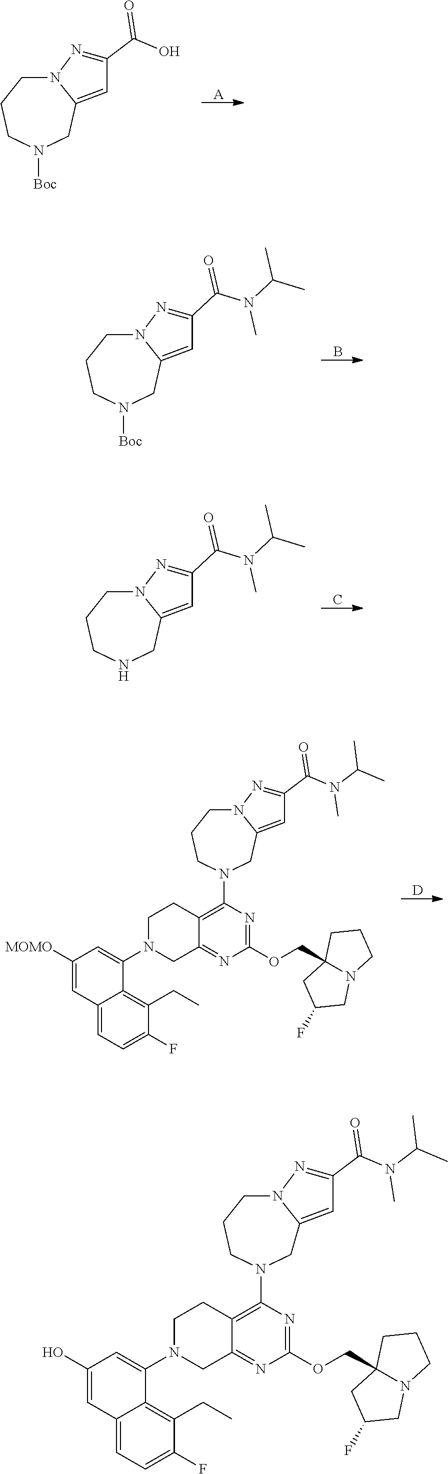

Synthesized according to Example 378. The title compound was obtained as orange solid (FA salt). ¹H NMR (400 MHz, METHANOL-d₄) δ=7.51 (dd, J=6.0, 9.2 Hz, 1H), 7.14 (t, J=9.2 Hz, 1H), 6.97 (s, 2H), 6.60-6.53 (m, 1H), 5.43-5.25 (m, 1H), 5.04-4.96 (m, 1H), 4.85-4.73 (m, 2H), 4.59-4.47 (m, 2H), 4.26-4.11 (m, 3H), 4.09-3.98 (m, 2H), 3.71-3.62 (m, 1H), 3.57-3.50 (m, 1H), 3.48-3.32 (m, 5H), 3.28-3.05 (m, 5H), 2.92 (s, 1H), 2.73 (br d, J=14.4 Hz, 1H), 2.41-2.22 (m, 3H), 2.21-2.12 (m, 1H), 2.10-2.00 (m, 3H), 1.97-1.88 (m, 1H), 1.25-1.17 (m, 6H), 1.10 (br t, J=6.8 Hz, 3H); LCMS (ESI, M+1): m/z=715.6.

### Example 382

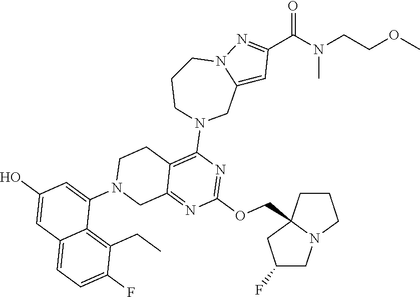 [SMILES: CCc1c(F)ccc2cc(O)cc(N3CCc4c(nc(OC[C@@]56CCCN5C[C@H](F)C6)nc4N4CCCn5nc(C(=O)N(C)CCOC)cc5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N-(2-methoxyethyl)-N-methyl-5,6,7,8-tetrahydro-4H-pyrazolo[1,5-a][1,4]diazepine-2-carboxamide

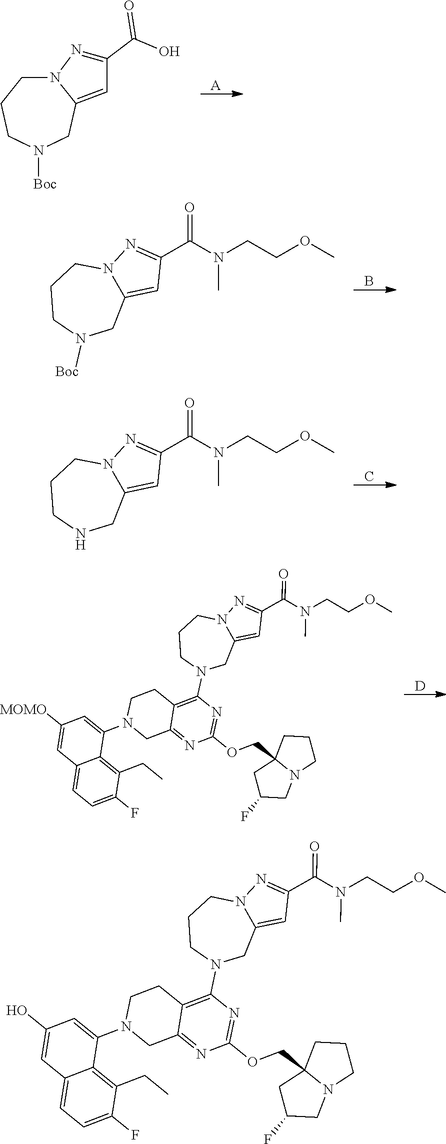

Synthesized according to Example 378. The title compound was obtained as white solid (FA salt). ¹H NMR (400 MHz, METHANOL-d4) δ=7.51 (dd, J=5.6, 8.8 Hz, 1H), 7.14 (t, J=9.2 Hz, 1H), 6.97 (s, 2H), 6.66-6.61 (m, 1H), 5.54-5.36 (m, 1H), 5.00-4.89 (m, 2H), 4.60-4.45 (m, 2H), 4.39-4.24 (m, 2H), 4.22-4.02 (m, 3H), 4.00-3.91 (m, 1H), 3.75-3.49 (m, 8H), 3.36 (s, 5H), 3.30-3.24 (m, 3H), 3.23-3.07 (m, 3H), 2.74 (br d, J=14.4 Hz, 1H), 2.60-2.38 (m, 2H), 2.35-2.14 (m, 4H), 2.13-1.98 (m, 2H), 1.10 (br t, J=6.8 Hz, 3H); LCMS (ESI, M+1): m/z=731.5.

### Example 383

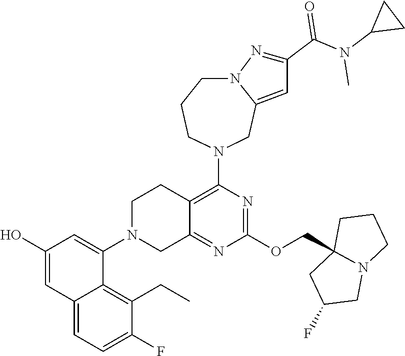 [SMILES: CCc1c(F)ccc2cc(O)cc(N3CCc4c(nc(OC[C@@]56CCCN5C[C@H](F)C6)nc4N4CCCn5nc(C(=O)N(C)C6CC6)cc5C4)C3)c12]

### N-cyclopropyl-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepine-2-carboxamide

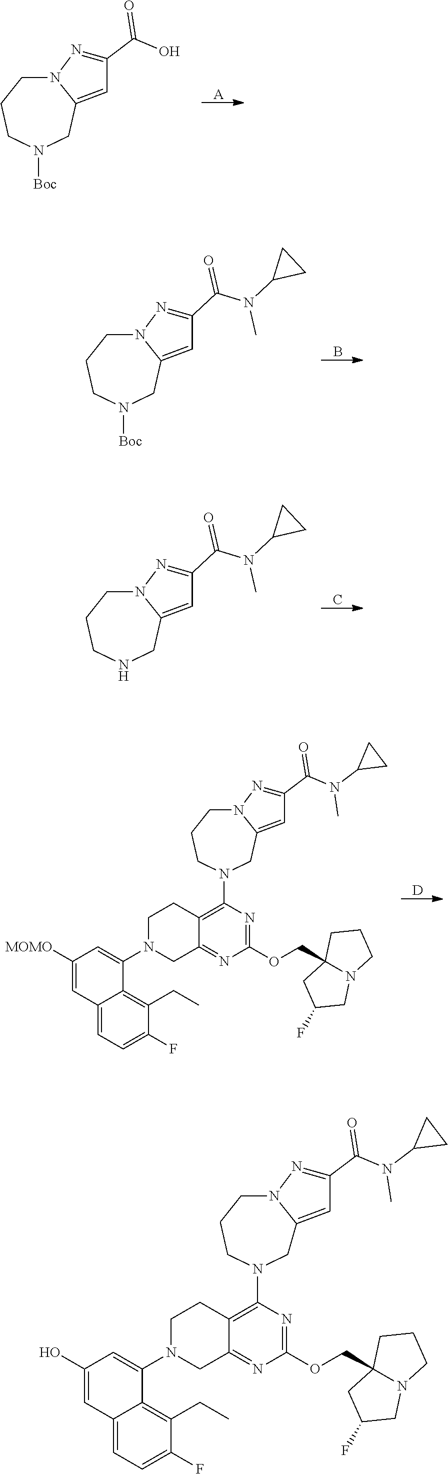

Synthesized according to Example 378. The title compound was obtained as yellow solid. ¹H NMR (400 MHz, METHANOL-d4) δ=7.51 (dd, J=5.2, 8.8 Hz, 1H), 7.14 (t, J=9.2 Hz, 1H), 6.96 (s, 2H), 6.56 (s, 1H), 5.39-5.14 (m, 1H), 5.01 (br d, J=16.6 Hz, 1H), 4.81 (br s, 1H), 4.54 (br s, 2H), 4.24-4.14 (m, 1H), 4.12-3.92 (m, 4H), 3.74-3.61 (m, 1H), 3.52 (br d, J=5.2 Hz, 1H), 3.39 (br s, 2H), 3.28-2.92 (m, 10H), 2.71 (br d, J=13.2 Hz, 1H), 2.38-2.17 (m, 2H), 2.15-2.01 (m, 3H), 2.00-1.78 (m, 3H), 1.10 (br t, J=7.2 Hz, 3H), 0.92-0.32 (m, 4H); LCMS (ESI, M+1): m/z=713.5.

### Example 384

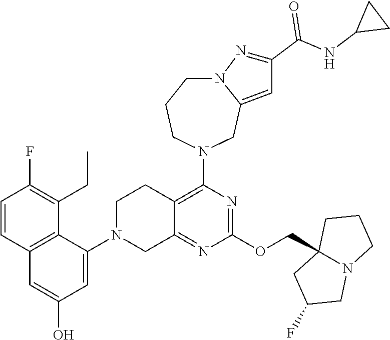 [SMILES: CCc1c(F)ccc2cc(O)cc(N3CCc4c(nc(OC[C@@]56CCCN5C[C@H](F)C6)nc4N4CCCn5nc(C(=O)NC6CC6)cc5C4)C3)c12]

### N-cyclopropyl-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepine-2-carboxamide

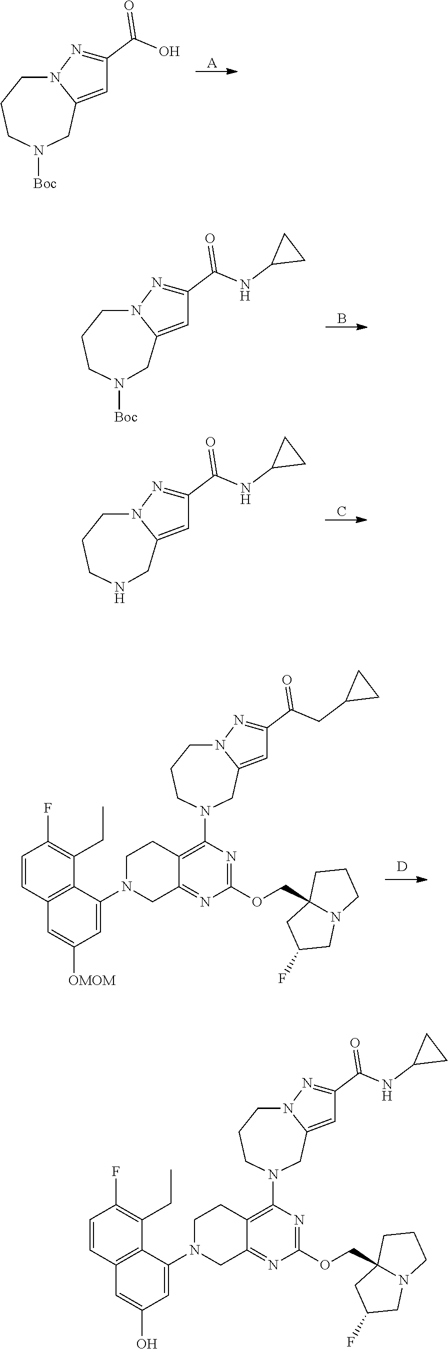

Synthesized according to Example 378. The title compound was obtained as yellow solid. ¹H NMR (400 MHz, MIETHANOL-d₄) δ=7.50-7.46 (m, 1H), 7.14-7.10 (m, 1H), 6.96 (s, 2H), 6.69 (d, J=1.2 Hz, 1H), 5.30-5.17 (m, 1H), 4.96-4.91 (m, 1H), 4.79-4.75 (m, 1H), 4.51-4.47 (m, 2H), 4.18-4.08 (m, 2H), 4.04-3.92 (m, 3H), 3.63 (br d, J=17.6 Hz, 1H), 3.49-3.46 (m, 1H), 3.43-3.40 (m, 2H), 3.25-3.07 (m, 5H), 2.99-2.93 (m, 1H), 2.80-2.74 (m, 1H), 2.66 (br d, J=14.4 Hz, 1H), 2.33-2.02 (m, 5H), 1.96-1.79 (m, 3H), 1.10 (t, J=7.2 Hz, 3H), 0.78-0.76 (m, 2H), 0.63-0.56 (m, 2H); LCMS (ESI, M+1): m/z=699.4.

### Example 385

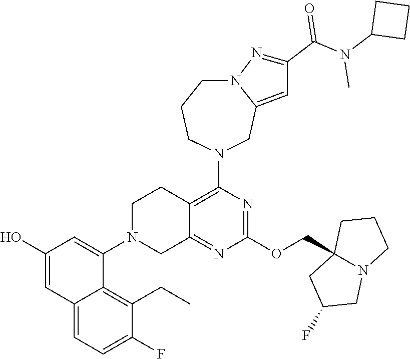 [SMILES: CCc1c(F)ccc2cc(O)cc(N3CCc4c(nc(OC[C@@]56CCCN5C[C@H](F)C6)nc4N4CCCn5nc(C(=O)N(C)C6CCC6)cc5C4)C3)c12]

### N-cyclobutyl-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N-methyl-56,7, 8-tetrahydro-4H-pyrazolo[1,5-a][1,4]diazepine-2-carboxamide

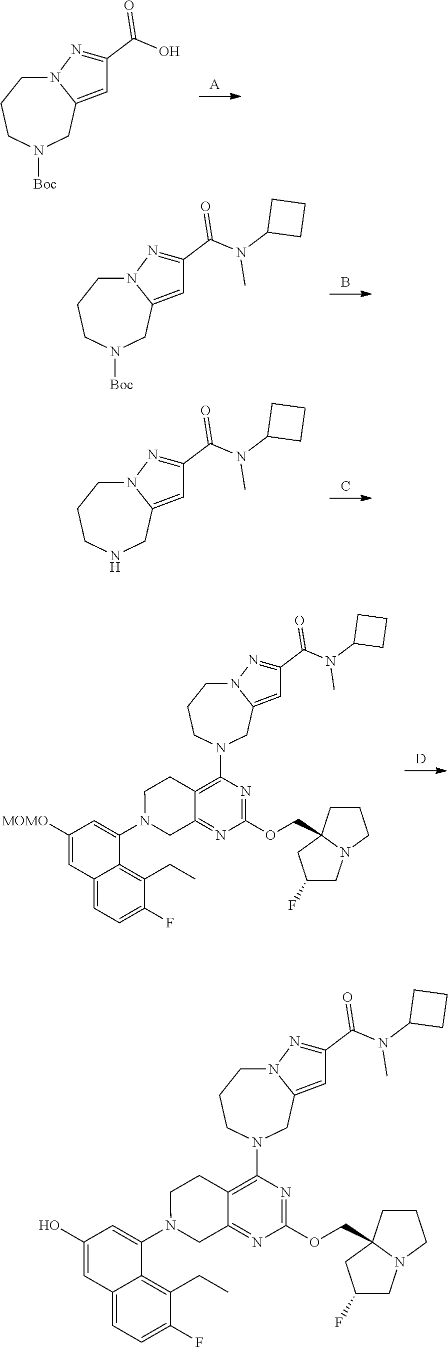

Synthesized according to Example 378. The title compound was obtained as orange solid (FA salt).¹H NMR (400 MHz, METHANOL-d4) δ=8.53 (s, 1H), 7.51 (dd, J=6.0, 8.8 Hz, 1H), 7.14 (t, J=9.4 Hz, 1H), 6.97 (s, 2H), 6.70-6.48 (m, 1H), 5.40-5.22 (m, 1H), 5.06-4.88 (m, 2H), 4.61-4.47 (m, 2H), 4.24-4.14 (m, 2H), 4.13-4.00 (m, 3H), 3.67 (br d, J=17.8 Hz, 1H), 3.54 (br d, J=10.0 Hz, 1H), 3.44-3.32 (m, 4H), 3.28-3.13 (m, 4H), 3.12-2.98 (m, 3H), 2.73 (br d, J=13.2 Hz, 1H), 2.40-2.17 (m, 6H), 2.16-1.48 (m, 9H), 1.10 (br t, J=7.2 Hz, 3H); LCMS (ESI, M+1): m/z=727.5.

### Example 386

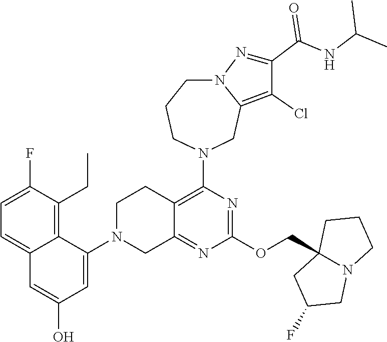 [SMILES: CCc1c(F)ccc2cc(O)cc(N3CCc4c(nc(OC[C@@]56CCCN5C[C@H](F)C6)nc4N4CCCn5nc(C(=O)NC(C)C)c(Cl)c5C4)C3)c12]

### 3-chloro-5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-N-isopropyl-5,6,7,8-tetrahydro-4H-pyrazolo[1,5-a][1,4]diazepine-2-carboxamide

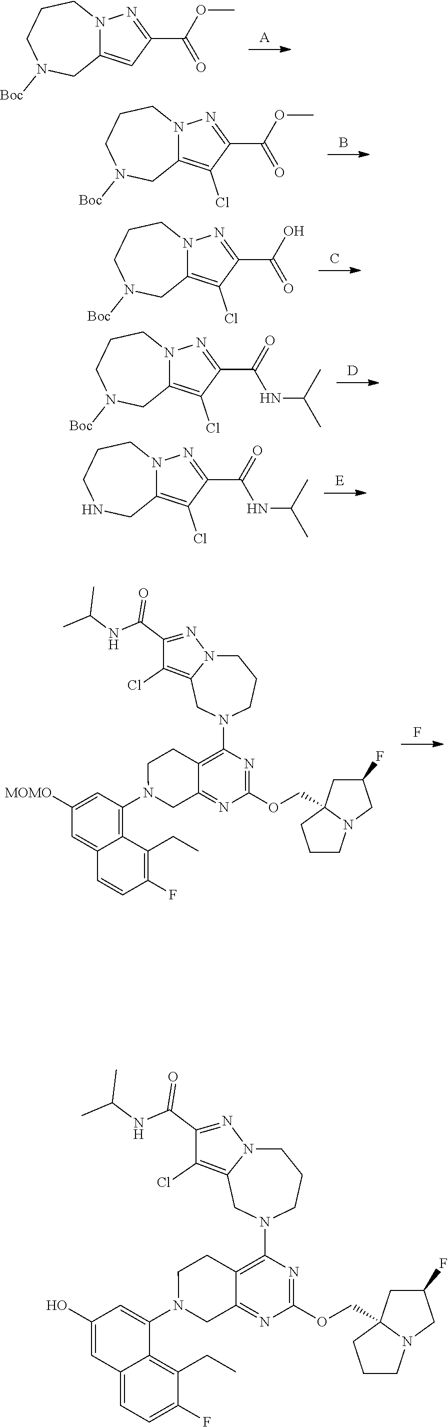

Step A. 5-tert-butyl 2-methyl 3-chloro-7,8-dihydro-4H-pyrazolo[1,5-a][1,4]diazepine-2,5(6H)-dicarboxylate: To a solution of 5-tert-butyl 2-methyl 7,8-dihydro-4H-pyrazolo[1,5-a][1,4]diazepine-2,5(6H)-dicarboxylate (8.00 g, 1.0 equiv) in DMF (80 mL) was added NCS (5.43 g, 1.5 equiv) at 0° C. The reaction was stirred at 55° C. for 1 hour. The mixture was quenched with H₂O (30 mL) and extracted with ethyl acetate (3×50 mL). The combined organic layers were washed with NaHCO₃ aqueous solution (3×50 mL), dried over anhydrous sodium sulfate, concentrated to afford the title compound (9.00 g, 96% yield) as yellow solid; LCMS (ESI, M+1): m/z=329.9.

Step B. 5-(tert-butoxycarbonyl)-3-chloro-5,6,7,8-tetrahydro-4H-pyrazolo[1,5-a][1,4]diazepine-2-carboxylic acid: To a solution of 5-tert-butyl 2-methyl 3-chloro-7,8-dihydro-4H-pyrazolo[1,5-a][1,4]diazepine-2,5(6H)-dicarboxylate (8.50 g, 1.0 equiv) in THE (60 mL) and H₂O (20 mL) was added NaOH (10.3 g, 10 equiv). The reaction was stirred at 20° C. for 0.5 hour. The mixture was adjusted to pH=4 with HCl (2 M) and extracted with ethyl acetate (3×100 mL). The combined organic layers were washed with brine (2×100 mL), dried over anhydrous sodium sulfate, concentrated to afford the title compound (8.40 g, 96% yield) as yellow solid; LCMS (ESI, M+1): m/z=316.0.

Step C. tert-butyl 3-chloro-2-(isopropylcarbamoyl)-7,8-dihydro-4H-pyrazolo[1,5-a][1,4]diazepine-5(6H)-carboxylate: To a solution of 5-(tert-butoxycarbonyl)-3-chloro-5,6,7,8-tetrahydro-4H-pyrazolo[1,5-a][1,4]diazepine-2-carboxylic acid (5.00 g, 1.0 equiv) and propan-2-amine (1.87 g, 2.0 equiv) in DMF (50 mL) were added HATU (9.03 g, 1.5 equiv) and N,N-diethylpropan-2-amine (6.14 g, 3.0 equiv). The reaction was stirred at 20° C. for 1 hour. The mixture was filtered and purified by reversed phase HPLC (0.1% FA condition) to afford the title compound (4.70 g, 82% yield) as pink solid.

Step D. 3-chloro-N-isopropyl-5,6,7,8-tetrahydro-4H-pyrazolo[1,5-a][1,4]diazepine-2-carboxamide: A solution of tert-butyl 3-chloro-2-(isopropylcarbamoyl)-7,8-dihydro-4H-pyrazolo[1,5-a][1,4]diazepine-5(6H)-carboxylate (2.5 g, 1.0 equiv) inHCl•dioxane (4 M, 12.5 mL, 7.1 equiv) was stirred at 20° C. for 0.5 hours. The mixture was concentrated to give a residue. The pH of the residue was adjusted to 8 with Na₂CO₃ in MeOH (30 mL). The mixture was filtered and purified by prep-HPLC (column: Welch Xtimate C18 250×70 mm×10 μm; mobile phase: [water (NH₃H20) -ACN]; B %: 9%-30%, 15 min) to afford the title compound (1.70 g, 93% yield) as yellow solid; LCMS (ESI, M+1): m/z=257.3.

The last two steps were performed according to Example 248. The title compound was obtained as yellow solid. ¹H NMR (400 MHz, CHLOROFORM-d) δ=7.35 (ddd, J=3.2, 5.6, 9.2 Hz, 11H), 7.06 (t, J=9.2 Hz, 1H), 6.89 (d, J=1.6 Hz, 1H), 6.85 (dd, J=2.4, 4.8 Hz, 11H), 6.59 (d, J=8.0 Hz, 1H), 5.32-5.13 (m, 1H), 4.65 (br d, J=5.2 Hz, 2H), 4.47-4.38 (m, 1H), 4.37-4.19 (m, 2H), 4.15-4.05 (m, 2H), 4.03-3.96 (m, 1H), 3.91-3.75 (m, 2H), 3.63 (br dd, J=6.4, 18.0 Hz, 1H), 3.35-3.24 (m, 4H), 3.23-3.03 (m, 3H), 3.01-2.89 (m, 2H), 2.46 (br dd, J=11.2, 14.0 Hz, 1H), 2.33-2.20 (m, 2H), 2.20-2.12 (m, 2H), 2.11-2.04 (m, 1H), 1.96-1.80 (m, 3H), 1.25 (d, J=6.4 Hz, 6H), 1.06 (t, J=7.2 Hz, 3H); LCMS (ESI, M+1): m/z=735.4

### Example 387

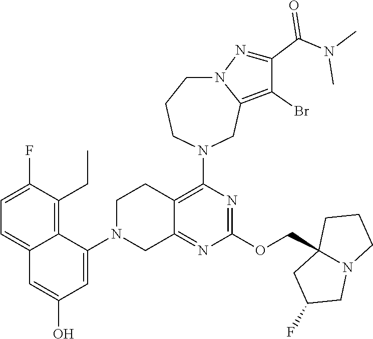 [SMILES: CCc1c(F)ccc2cc(O)cc(N3CCc4c(nc(OC[C@@]56CCCN5C[C@H](F)C6)nc4N4CCCn5nc(C(=O)N(C)C)c(Br)c5C4)C3)c12]

### 3-bromo-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide

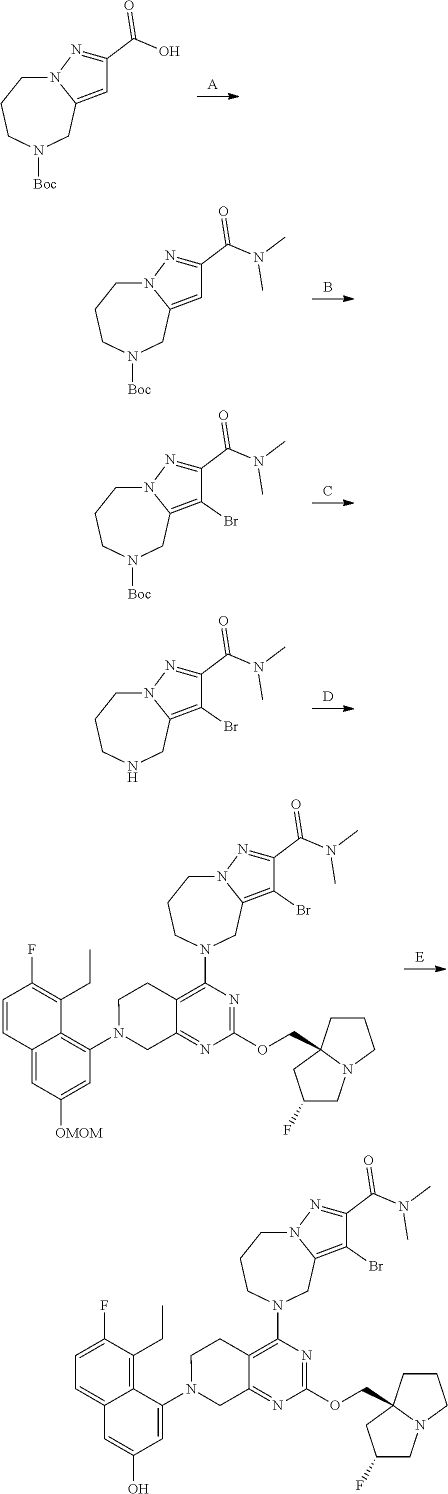

Step A. tert-butyl 2-(dimethylcarbamoyl)-7,8-dihydro-4H-pyrazolo[1,5-a][1,4]diazepine-5(6H)-carboxylate: To a solution of 5-(tert-butoxycarbonyl)-5,6,7,8-tetrahydro-4H-pyrazolo[1,5-a][1,4]diazepine-2-carboxylic acid (3.00 g, 1.0 equiv) and dimethylamine (2 M, 16.0 mL, 3.0 equiv) in DMF (30 mL) were added HATU (6.08 g, 1.5 equiv) and N,N-diethylpropan-2-amine (4.13 g, 3.0 equiv). The reaction was stirred at 20° C. for 1 hour. The mixture was diluted with water (50 mL) and extracted with EtOAc (2×40 mL). The combined organic layers were washed with brine (3×40 mL), dried over anhydrous sodium sulfate, concentrated, and purified with column chromatography (silicon dioxide, dichloromethane/methanol=I/O to 10/1). The cut fraction was filtered and concentrated under reduced pressure to afford the title compound (3.20 g, 96% yield) as a yellow solid; LCMS (ESI, M+1): m/z=309.0.

Step B. tert-butyl 3-bromo-2-(dimethylcarbamoyl)-7,8-dihydro-4H-pyrazolo[1,5-a][1,4]diazepine-5(6H)-carboxylate: To a solution of tert-butyl 2-(dimethylcarbamoyl)-7,8-dihydro-4H-pyrazolo[1,5-a][1,4]diazepine-5(6H)-carboxylate (3.20 g, 1.0 equiv) in DMF (32 mL) was added NBS (2.77 g, 1.5 equiv). The reaction was stirred at 60° C. for 1 hour. The mixture was diluted with water (80 mL) and extracted with EtOAc (2×40 mL). The combined organic layers were washed with brine (2×50 mL), dried over anhydrous sodium sulfate, concentrated, and purified with column chromatography (silicon dioxide, dichloromethane/methanol=I/O to 10/1). The cut fraction was filtered and concentrated under reduced pressure to afford the title compound (3.90 g, 97% yield) as a yellow solid; LCMS (ESI, M+1, M+3): m/z=386.9, 388.9.

Step C. 3-bromo-N,N-dimethyl-5,6,7,8-tetrahydro-4H-pyrazolo[1,5-a][1,4]diazepine-2-carboxamide: To a solution of tert-butyl 3-bromo-2-(dimethylcarbamoyl)-7,8-dihydro-4H-pyrazolo[1,5-a][1,4]diazepine-5(6H)-carboxylate (3.90 g, 1.0 equiv) in acetonitrile (20 mL) was added HCl•dioxane (4 M, 39.0 mL, 15 equiv). The reaction was stirred at 25° C. for 1 hour. To the mixture was added potassium carbonate saturated aqueous solution to adjust the pH >8. The mixture was extracted with dichloromethane (2×100 mL). The combined organic layers were washed with brine (20 mL), dried over anhydrous sodium sulfate, and concentrated to afford the title compound (2.30 g, 80% yield) as a yellow solid.

The last two steps were performed according to Example 248. The title compound was obtained as off-white solid. ¹H NMR (400 MHz, METHANOL-d4) δ=7.49 (dd, J=6.0, 8.8 Hz, 1H), 7.13 (t, J=9.6 Hz, 1H), 7.00-6.80 (m, 2H), 5.36-5.13 (m, 1H), 5.00-4.90 (m, 1H), 4.76 (dd, J=7.2, 16.6 Hz, 1H), 4.56-4.37 (m, 2H), 4.14-3.97 (m, 4H), 3.92 (ddd, J=4.4, 9.2, 13.6 Hz, 1H), 3.67 (br dd, J=5.2, 18.0 Hz, 1H), 3.50-3.36 (m, 3H), 3.29-3.01 (m, 11H), 3.00-2.92 (m, 1H), 2.66 (br d, J=15.2 Hz, 1H), 2.40-2.02 (m, 5H), 1.99-1.77 (m, 3H), 1.10 (t, J=7.2 Hz, 3H); LCMS (ESI, M+1, M+3): m/z=765.2, 767.2.

### Example 388

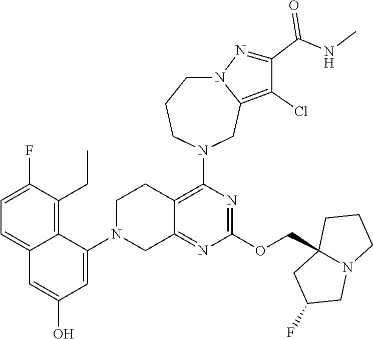 [SMILES: CCc1c(F)ccc2cc(O)cc(N3CCc4c(nc(OC[C@@]56CCCN5C[C@H](F)C6)nc4N4CCCn5nc(C(=O)NC)c(Cl)c5C4)C3)c12]

### 3-chloro-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepine-2-carboxamide

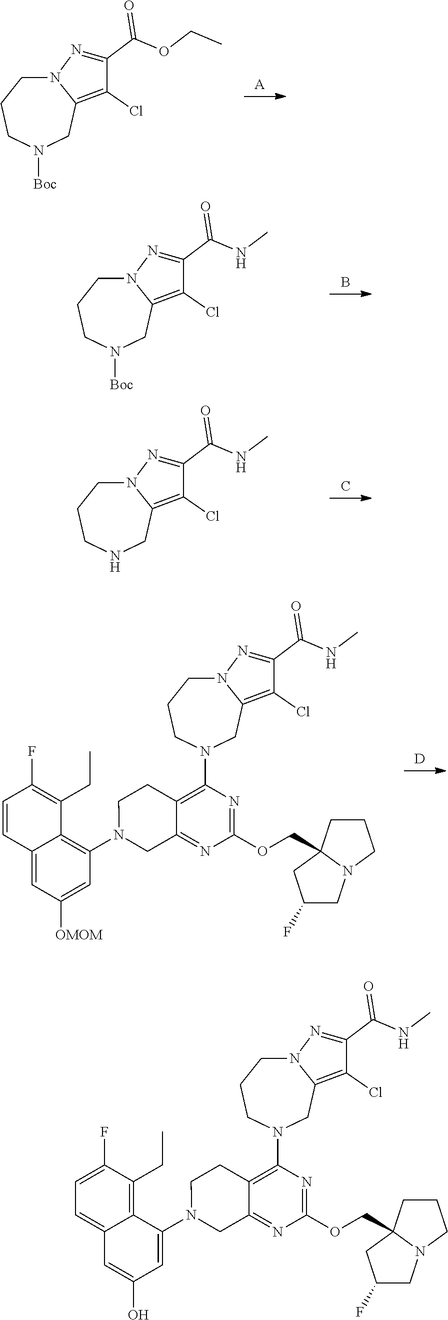

Synthesized according to Example 367 Steps B-E. The title compound was obtained as orange solid (FA salt). H NMR (400 MHz, chloroform-d) δ=7.38 (ddd, J=2.4, 6.0, 8.8 Hz, 1H), 7.07 (t, J=9.2 Hz, 1H), 6.95-6.86 (m, 2H), 6.81 (t, J=5.2 Hz, 1H), 5.40-5.17 (m, 1H), 4.71-4.56 (m, 2H), 4.46-4.27 (m, 2H), 4.24-4.00 (m, 3H), 3.88-3.72 (m, 3H), 3.52-3.36 (m, 2H), 3.36-3.27 (m, 3H), 3.26 (br s, 1H), 3.02 (br dd, J=4.8, 9.2 Hz, 2H), 2.98-2.87 (m, 4H), 2.50-2.30 (m, 2H), 2.29-2.09 (m, 4H), 2.05-1.83 (m, 3H), 1.07 (t, J=7.2 Hz, 3H); ¹⁹F NMR (400 MHz, chloroform-d) δ=−120.898,-172.435; LCMS (ESI,M+1): m/z=707.6.

### Example 389

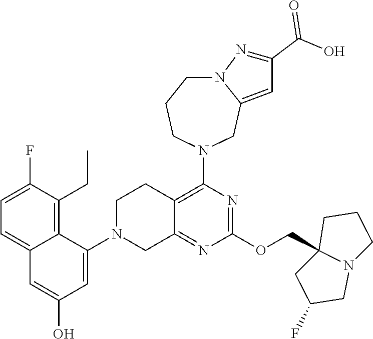 [SMILES: CCc1c(F)ccc2cc(O)cc(N3CCc4c(nc(OC[C@@]56CCCN5C[C@H](F)C6)nc4N4CCCn5nc(C(=O)O)cc5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepine-2-carboxylic acid

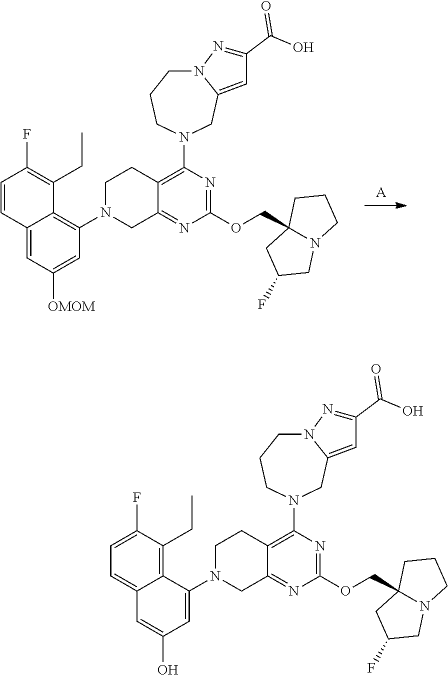

Step A. 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepine-2-carboxylic acid: A mixture of 5-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)5,6,7,8-tetrahydro-4H-pyrazolo[1,5-a][1,4]diazepine-2-carboxylic acid (90.0 mg, 1.0 equiv) in HCl/MeOH (4 M, 23 equiv) was stirred at 25° C. for 0.5 hours. The mixture was concentrated under vacuum. The pH of the residue was adjusted to 8 by NaHCO₃ solution. The mixture was extracted with EtOAc. (2×5 mL). The combined organic layers were dried over anhydrous sodium sulfate, concentrated, and purified with prep-HPLC column: Phenomenex C18 150*25 mm*10 um;mobile phase: [water(NH₄HCO₃)-ACN];B %: 20%-50%,8 min to afford the title compound (12 mg, 14% yield) as yellow solid; ¹H NMR (400 MHz, METHANOL-d4) δ=7.61 (s, 1H), 7.14 (t, J=9.2 Hz, 1H), 6.97 (s, 2H), 6.65 (br d, J=2.8 Hz, 1H), 5.64-5.41 (m, 1H), 4.92 (br d, J=4.4 Hz, 1H), 4.81 (br s, 1H), 4.55-4.25 (m, 4H), 4.16-3.99 (m, 3H), 3.88-3.63 (m, 4H), 3.55 (br d, J=9.2 Hz, 1H), 3.42-3.33 (m, 3H), 3.26-3.14 (m, 2H), 2.80-2.45 (m, 3H), 2.43-2.33 (m, 1H), 2.32-2.19 (m, 3H), 2.18-1.94 (m, 2H), 1.10 (br t, J=7.2 Hz, 3H); LCMS (ESI, M+1): m/z=660.2

### Example 390

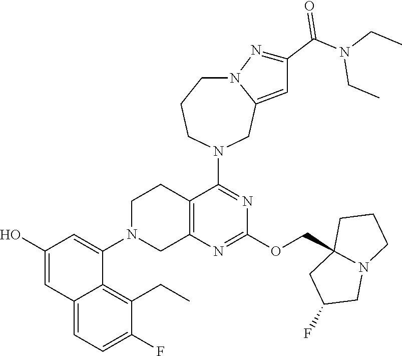 [SMILES: CCc1c(F)ccc2cc(O)cc(N3CCc4c(nc(OC[C@@]56CCCN5C[C@H](F)C6)nc4N4CCCn5nc(C(=O)N(CC)CC)cc5C4)C3)c12]

### N,N-diethyl-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepine-2-carboxamide

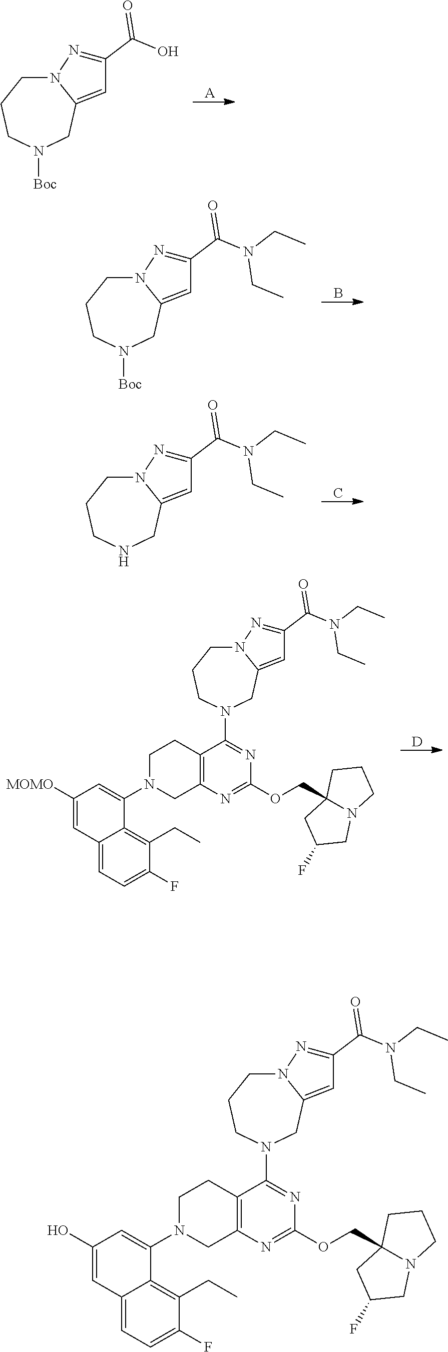

Synthesized according to Example 378. The title compound was obtained as orange solid. ¹H NMR (400 MHz, METHANOL-d4) δ=7.56-7.47 (m, 1H), 7.14 (t, J=9.2 Hz, 1H), 6.97 (s, 2H), 6.59 (d, J=3.2 Hz, 1H), 5.47-5.29 (m, 1H), 5.02-4.94 (m, 1H), 4.88 (br d, J=8.4 Hz, 1H), 4.56-4.49 (m, 2H), 4.21 (s, 3H), 4.03 (s, 2H), 3.75-3.64 (m, 3H), 3.51 (br d, J=7.2 Hz, 6H), 3.43-3.34 (m, 2H), 3.28-3.13 (m, 3H), 2.74 (br d, J=14.0 Hz, 1H), 2.50-2.35 (m, 1H), 2.35-2.25 (m, 2H), 2.25-2.17 (m, 1H), 2.16-2.08 (m, 2H), 2.07 (br s, 2H), 1.22 (t, J=7.2 Hz, 6H), 1.10 (br d, J=2.4 Hz, 3H), LCMS (ESI, M+1): m/z=715.3.

### Example 391

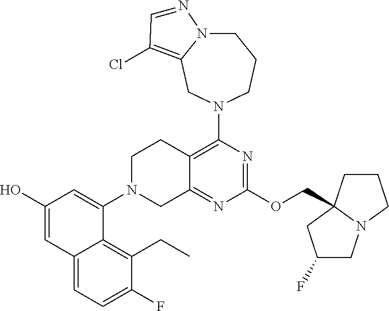 [SMILES: CCc1c(F)ccc2cc(O)cc(N3CCc4c(nc(OC[C@@]56CCCN5C[C@H](F)C6)nc4N4CCCn5ncc(Cl)c5C4)C3)c12]

### 4-(4-(3-chloro-7,8-dihydro-4H-[1,2,3]triazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

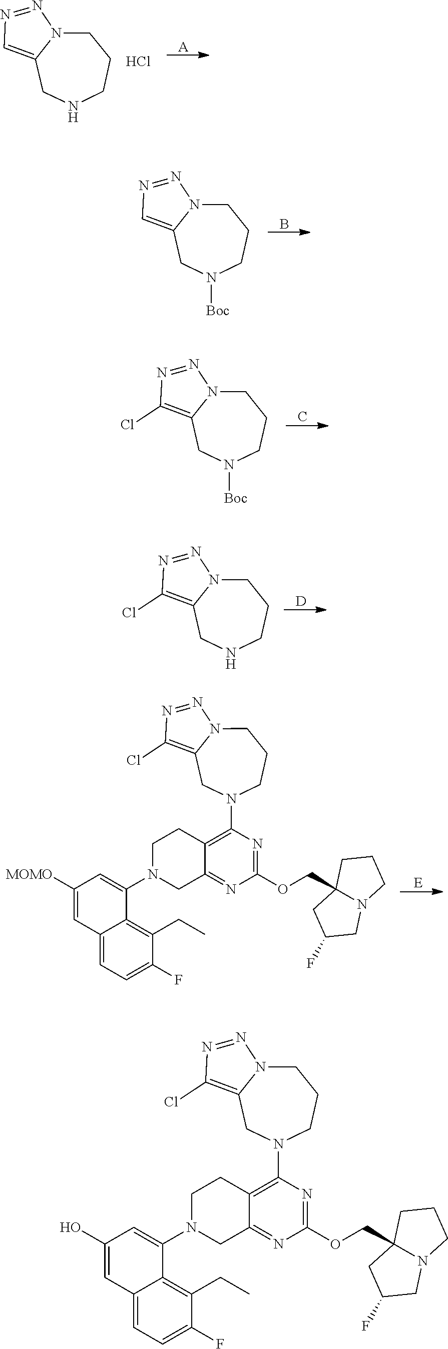

Step A. tert-butyl 7,8-dihydro-4H-[1,2,3]triazolo[1,5-a][1,4]diazepine-5(6H)-carboxylate: To a solution of 5,6,7,8-tetrahydro-4H-[1,2,3]triazolo[1,5-a][1,4]diazepine (1.00 g, 1.0 equiv, HCl) in tert-butoxycarbonyl tert-butyl carbonate (10 mL) was added DMAP (770 mg, 1.1 equiv). The mixture was stirred at 80° C. for 12 hours. The mixture was concentrated, diluted with water (10 mL), and extracted with ethyl acetate (10 mL×2). The combined organic layers were washed with brine (10 mL), dried over anhydrous sodium sulfate, and concentrated to afford the title compound. (1.0 g, 73% yield) as yellow solid; ¹H NMR (400 MHz, CHLOROFORM-d) δ=7.65-7.47 (m, 1H), 4.68-4.62 (m, 2H), 4.60-4.48 (m, 2H), 3.80-3.72 (m, 2H), 2.00-1.93 (m, 2H), 1.40 (s, 9H) LCMS (ESI, M+1): m/z=239.1.

Step B. tert-butyl 3-chloro-7,8-dihydro-4H-[1,2,3]triazolo[1,5-a][1,4]diazepine-5(6H)-carboxylate: To a solution of tert-butyl 7,8-dihydro-4H-[1,2,3]triazolo[1,5-a][1,4]diazepine-5(6H)-carboxylate (950 mg, 1.0 equiv) in MeCN (10 mL) was added NCS (639 mg, 1.2 equiv). The reaction was stirred at 60° C. for 3 hours. The mixtue was diluted with water (10 mL) and extracted with ethyl acetate (10 mL×3). The organic layers were washed with brine(10 mL), dried over anhydrous sodium sulfate, concentrated, and purified with reversed phase flash chromatography [C18, 0.1% formic acid condition] to afford the title compound (700 mg, 64% yield) as white solid; ¹H NMR (400 MHz, CHLOROFORM-d) δ=4.67-4.59 (m, 2H), 4.58-4.51 (m, 2H), 3.81-3.72 (m, 2H), 2.03-1.97 (m, 2H), 1.44 (s, 9H); LCMS (ESI, M+1): m/z=273.0.

Step C. 3-chloro-5,6,7,8-tetrahydro-4H-[1,2,3]triazolo[1,5-a][1,4]diazepine: To a solution of tert-butyl 3-chloro-7,8-dihydro-4H-[1,2,3]triazolo[1,5-a][1,4]diazepine-5(6H)-carboxylate (700 mg, 1.0 equiv) in MeOH (4 mL) was added HCl/dioxane (8 mL). The reaction was stirred at 0° C. for 1 hour. The mixture was concentrated and stirred with NaHCO₃ (258 mg,) in MeOH (5 mL) at 25° C. for 1 hour. The mixture was filtered and concentrated under reduced pressure to afford the title compound (480 mg, 90% yield) as white solid.

The last two steps were performed according to Example 248. The title compound was obtained as yellow solid (FA salt). ¹H NMR (400 MHz, METHANOL-d4) δ=7.55-7.47 (m, 1H), 7.14 (t, J=9.2 Hz, 1H), 6.97 (br d, J=2.4 Hz, 2H), 5.53-5.31 (m, 1H), 4.89 (br s, 2H), 4.79-4.59 (m, 2H), 4.25 (dd, J=5.6, 11.2 Hz, 1H), 4.18-4.03 (m, 4H), 3.74-3.64 (m, 1H), 3.60-3.48 (m, 4H), 3.42-3.35 (m, 2H), 3.28-3.14 (m, 3H), 2.71 (br d, J=14.4 Hz, 1H), 2.42 (br s, 3H), 2.23-2.09 (m, 4H), 1.99 (br d, J=2.5 Hz, 1H), 1.10 (t, J=7.3 Hz, 3H); LCMS (ESI, M+1): m/z=651.3.

### Example 392

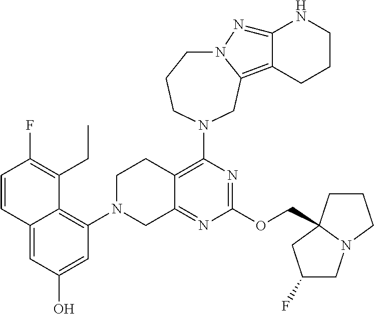 [SMILES: CCc1c(F)ccc2cc(O)cc(N3CCc4c(nc(OC[C@@]56CCCN5C[C@H](F)C6)nc4N4CCCn5nc6c(c5C4)CCCN6)C3)c12]

### 5-ethyl-6-fluoro-4-(2-(((2R,7aS)-2-fluorohexahydro-1H-pyrrolizin-7a-yl)methoxy)-4-(3,4,8,9-tetrahydro-1H-pyrido[2′,3′:3,4]pyrazolo[1,5-a][1,4]diazepin-6(2H,5H,7H)-yl)-5,6-dihydropyrido[3,4-d]pyrimidin-7(8H)-yl)naphthalen-2-ol

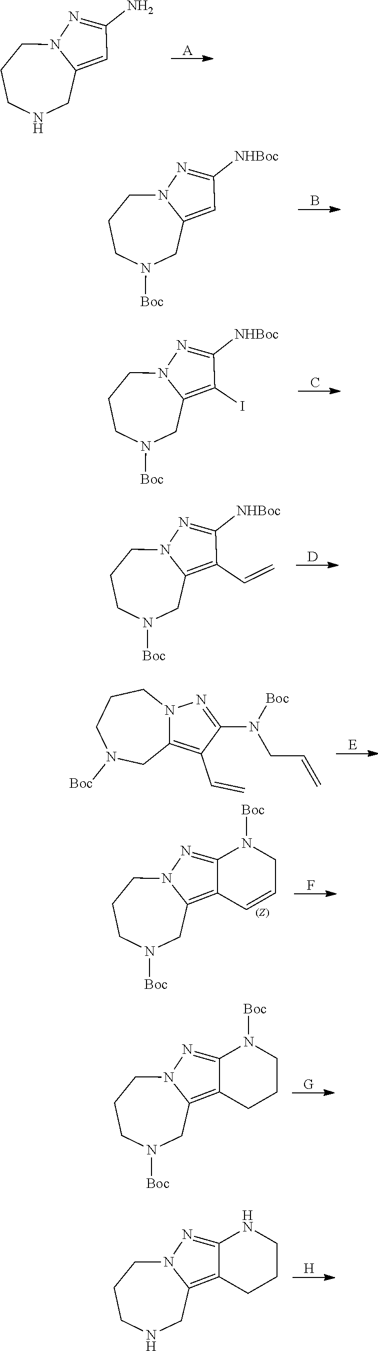

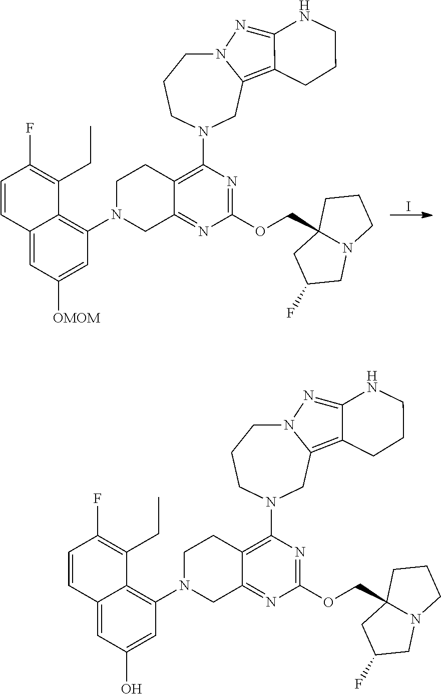

Step A. tert-butyl 2-(tert-butoxycarbonylamino)-4,6,7,8-tetrahydropyrazolo[1,5-a][1,4]diazepine-5-carboxylate: To a flask containing 5,6,7,8-tetrahydro-4H-pyrazolo[1,5-a][1,4]diazepin-2-amine (2.0 g, 1.0 equiv) was added (Boc)20 (19.0 g, 20 mL). The mixture was stirred at 60° C. for 3 hours. The crude product was triturated with Petroleum ether (50 mL) for 30 minutes at 25° C. The reaction was filtered and concentrated to afford the title compound (4.0 g, 86% yield) as white solid; LCMS (ESI, M+1): m/z=353.2.

Step B. tert-butyl2-(tert-butoxycarbonylamino)-3-iodo-4,6,7,8-tetrahydropyrazolo[1,5-a][1,4]diazepine-5-carboxylate: To a solution of tert-butyl 2-(tert-butoxycarbonylamino)-4,6,7,8-tetrahydropyrazolo[1,5-a][1,4]diazepine-5-carboxylate (3.80 g, 1.0 equiv) in CH3COOH (40 mL) was added NIS (4.85 g, 2.0 equiv). The mixture was stirred at 80° C. for 1 hour. The reaction was quenched with saturated NaHCO₃ solution (500 mL) at 0° C. The reaction was extracted with EtOAc (3×100 mL). The combined organic layers were washed with brine (200 mL), dried over anhydrous sodium sulfate, concentrated, and purified with prep-HPLC [C18, 0.1% formic acid condition] to afford the title compound (4.0 g, 78% yield) as yellow solid; LCMS (ESI, M+1): m/z=479.1.

Step C. tert-butyl 2-(tert-butoxycarbonylamino)-3-vinyl-4,6,7,8-tetrahydropyrazolo[1,5-a][1,4]diazepine-5-carboxylate: To a solution of tert-butyl 2-(tert-butoxycarbonylamino)-3-iodo-4,6,7,8-tetrahydropyrazolo[1,5-a][1,4]diazepine-5-carboxylate (2.40 g, 1.0 equiv) in dioxane (24 mL) and H₂O (2.4 mL) were added Pd(dppf)C12 (183.57 mg, 0.05 equiv), potassium;ethenyl(trifluoro)boranuide (2.69 g, 4.0 equiv), and Cs2CO₃ (8.17 g, 5.0 equiv). The mixture was stirred at 90° C. for 12 hours under N2. The mixture was poured into water (20 mL) and extracted with EtOAc (3×20 mL). The combined organic layers were dried over anhydrous sodium sulfate, concentrated, and purified by silica gel chromatography [SiO2, Petroleum ether/Ethyl acetate=I/O to 1/1] to afford the title compound (1.3 g, 68% yield) as yellow oil; LCMS (ESI, M+1): m/z=379.3.

Step D. tert-butyl 2-(allyl(tert-butoxycarbonyl)amino)-3-vinyl-7,8-dihydro-4H-pyrazolo[1,5-a][1,4]diazepine-5(6H)-carboxylate: To a solution of tert-butyl 2-(tert-butoxycarbonylamino)-3-vinyl-4,6,7,8-tetrahydropyrazolo[1,5-a][1,4]diazepine-5-carboxylate (1.3 g, 1.0 equiv) in THE (13 mL) was added NaH (412 mg, 60% purity, 3.0 equiv) slowly at 25° C. for 1 hour. Then 3-bromoprop-1-ene (2.08 g, 5.0 equiv) was added. The reaction was stirred at 25° C. for 11 hours. The mixture was poured into water (4 mL) and filtered. The filtrate was extracted with EtOAc (3×20 mL). The combined organic layers were dried over anhydrous sodium sulfate, concentrated, and purified with prep-HPLC [C18, 0.1% formic acid condition] to afford the title compound (1.30 g, 89% yield) as white solid; LCMS (ESI, M+1): m/z=419.3.

Step E. di-tert-butyl 5,7,8,9-tetrahydro-1H-pyrido[2′,3′:3,4]pyrazolo[1,5-a][1,4]diazepine-1,6(2H)-dicarboxylate: To a solution of tert-butyl 2-(allyl(tert-butoxycarbonyl)amino)-3-vinyl-7,8-dihydro-4H-pyrazolo[1,5-a][1,4]diazepine-5(6H)-carboxylate (2.2 g, 1.0 equiv) in DCM (110 mL) was added benzylidene-[1,3-bis(2,4,6-trimethylphenyl)imidazolidin-2-ylidene]-dichloro-ruthenium;tricyclohexylphosphane (892 mg, 0.2 equiv). The mixture was stirred at 45° C. for 12 hours. The mixture was poured into water (60 mL) and filtered. The filtrate was extracted with DCM (3×100 mL). The combined organic layers were dried over anhydrous sodium sulfate, concentrated, and purified by silica gel chromatography [SiO₂, Petroleum ether/Ethyl acetate=I/O to 1/1] to afford the title compound (417 mg, 20% yield) as white solid; ¹H NMR (400 MHz, CHLOROFORM-d) δ=6.43-6.23 (m, 1H), 5.51 (s, 1H), 4.51-4.28 (m, 5H), 4.13 (s, 1H), 3.68 (s, 2H), 2.05 (d, J=4.4 Hz, 2H), 1.89 (s, 2H), 1.57-1.34 (m, 18H); LCMS (ESI, M+1): m/z=391.2.

Step F. di-tert-butyl 3,4,5,7,8,9-hexahydro-1H-pyrido[2′,3′:3,4]pyrazolo[1,5-a][1,4]diazepine-1,6(2H)-dicarboxylate: To a solution of di-tert-butyl 5,7,8,9-tetrahydro-1H-pyrido[2′,3′:3,4]pyrazolo[1,5-a][1,4]diazepine-1,6(2H)-dicarboxylate (400 mg, 1.0 equiv) in MeOH (4 mL) was added Pd/C (40 mg, 10% purity) under N2 atmosphere. The suspension was degassed and purged with H2 3 times. The mixture was stirred under H2 (15 Psi) at 25° C. for 12 hours. The reaction was filtered, concentrated, and purified with reversed phase flash chromatography [C18, 0.1% formic acid condition] to afford the title compound (289 mg, 72% yield) as yellow solid; ¹H NMR (400 MHz, METHANOL-d4) δ=4.44-4.37 (m, 2H), 4.34-4.24 (m, 2H), 3.72 (d, J=3.2 Hz, 4H), 2.63-2.54 (m, 2H), 1.92-1.81 (m, 4H), 1.54 (s, 9H), 1.43 (s, 9H); LCMS (ESI, M+1): m/z=393.3.

Step G. 2,3,4,5,6,7,8,9-octahydro-1H-pyrido[2′,3′:3,4]pyrazolo[1,5-a][1,4]diazepine: To a solution of di-tert-butyl 3,4,5,7,8,9-hexahydro-1H-pyrido[2′,3′:3,4]pyrazolo[1,5-a][1,4]diazepine-1,6(2H)-dicarboxylate (110 mg, 1.0 equiv) in MeOH (1 mL) was added HCl•MeOH (4 M, 5 mL) at 0° C. The mixture was stirred at 25° C. for 1 hour. The reaction was quenched by addition saturated NaHCO₃ solution (4 mL) at 0° C. The reaction was extracted with EtOAc (3×5 mL). The combined organic layers were dried over anhydrous sodium sulfate and concentrated to afford the title compound (50.0 mg, crude) as white solid; LCMS (ESI, M+1): m/z=193.0.

The last two steps were performed according to Example 248. The title compound was obtained as pink solid. ¹H NMR (400 MHz, METHANOL-d4) δ=7.52-7.49 (m, 1H), 7.15 (t, J=9.4 Hz, 1H), 6.95 (s, 2H), 5.37-5.15 (m, 1H), 4.61-4.56 (m, 1H), 4.21 (t, J=5.2 Hz, 2H), 4.16-3.98 (m, 4H), 3.91-3.82 (m, 1H), 3.69 (d, J=18.0 Hz, 1H), 3.57-3.34 (m, 4H), 3.28-3.10 (m, 7H), 2.99-2.96 (m, 1H), 2.73-2.65 (m, 1H), 2.62-2.42 (m, 2H), 2.30-2.17 (m, 2H), 2.17-2.03 (m, 3H), 2.00-1.74 (m, 5H), 1.10 (t, J=7.2 Hz, 3H); LCMS (ESI, M+1): m/z=671.3.

### Example 393

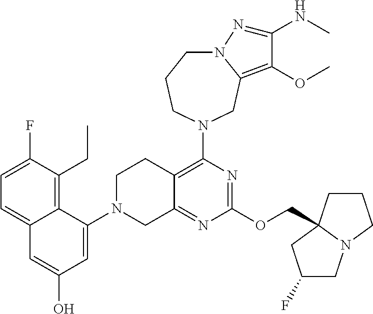 [SMILES: CCc1c(F)ccc2cc(O)cc(N3CCc4c(nc(OC[C@@]56CCCN5C[C@H](F)C6)nc4N4CCCn5nc(NC)c(OC)c5C4)C3)c12]

### 5-ethyl-6-fluoro-4-(2-(((2R,7aS)-2-fluorohexahydro-1H-pyrrolizin-7a-yl)methoxy)-4-(3-methoxy-2-(methylamino)-7,8-dihydro-4H-pyrazolo[1,5-a][1,4]diazepin-5(6H)-yl)-5,6-dihydropyrido[3,4-d]pyrimidin-7(8H)-yl)naphthalen-2-ol

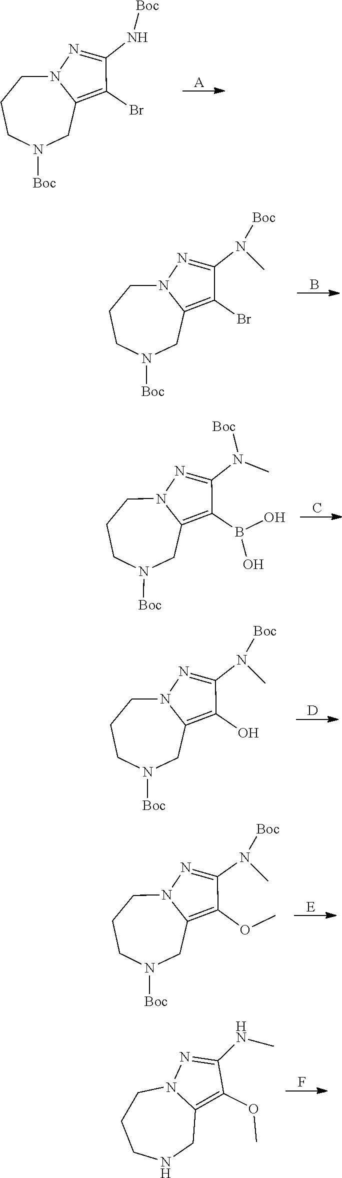

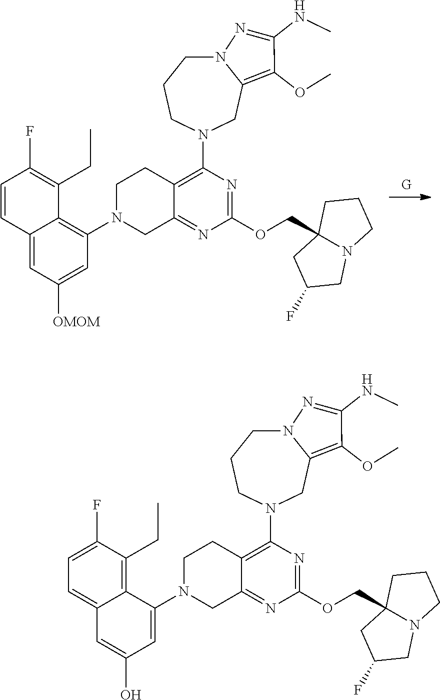

Step A. tert-butyl 3-bromo-2-((tert-butoxycarbonyl)(methyllamino)-7,8-dihydro-4H-pyrazolo[1,5-a][1,4]diazepine-5(6H)-carboxylate: To a solution of tert-butyl 3-bromo-2-((tert-butoxycarbonyl)amino)-7,8-dihydro-4H-pyrazolo[1,5-a][1,4]diazepine-5(6H)-carboxylate (3.30 g, 1.0 equiv) in THE (40 mL) was added NaH (749 mg, 60% purity, 2.0 equiv) at 0° C. After addition, the mixture was stirred at 0° C. for 0.5 hours, and then Mel (6.65 g, 5.0 equiv) was added dropwise at 0° C. The resulting mixture was stirred at 20° C. for 1 hour. The mixture was quenched with water (50 mL) and extracted with ethyl acetate (2×20 mL). The combined organic layers were washed with brine (20 mL), dried over anhydrous sodium sulfate, filtered, and concentrated to give a residue. The residue was purified by column chromatography (SiO2, petroleum ether/ethyl acetate 10/1 to 1/1) to afford the title compound (1.9 g, 55% yield) as yellow solid; LCMS (ESI, M-77): m/z=367.3.

Step B. (5-(tert-butoxycarbonyl)-2-((tert-butoxycarbonyl)(methyl)amino)-5,6,7,8-tetrahydro-4H-pyrazolo[1,5-a][1,4]diazepin-3-yl)boronic acid: To a solution of tert-butyl 3-bromo-2-[tert-butoxycarbonyl(methyl)amino]-4,6,7,8-tetrahydropyrazolo[1,5-a][1,4]diazepine-5-carboxylate (200 mg, 1.0 equiv) in THE (2 mL) was added n-BuLi (2.5 M, 2.0 equiv) dropwise at −78° C. The mixture was stirred at −78° C. for 1 hour. Then B(OMe)₃ (233 mg, 5.0 equiv) was added into the mixture and stirred at 20° C. for 2 hours. The mixture was diluted with water (10 mL) and extracted with ethyl acetate (2×10 mL). The combined organic layers were dried over anhydrous sodium sulfate, concentrated, purified by reversed phase flash chromatography [water (0.1% FA)/acetonitrile]. The desired fractions were neutralized with solid NaHCO₃, concentrated, and extracted with ethyl acetate (2×20 mL). The combined organic layers were dried over anhydrous sodium sulfate and concentrated to afford the title compound (173 mg, 78% yield) as yellow solid; LCMS (ESI, M+23): m/z=433.2.

Step C. tert-butyl 2-((tert-butoxycarbonyl)(methyl)amino)-3-hydroxy-7,8-dihydro-4H-pyrazolo[1,5-a][1,4]diazepine-5(6H)-carboxylate: To a solution of (5-(tert-butoxycarbonyl)-2-((tert-butoxycarbonyl)(methyl)amino)-5,6,7,8-tetrahydro-4H-pyrazolo[1,5-a][1,4]diazepin-3-yl)boronic acid (260 mg, 1.0 equiv) in THE (10 mL) was added H₂O₂ (4.77 g, 30% purity, 71 equiv). The reaction was stirred at 50° C. for 16 hours. The mixture was quenched with Na2SO3 (10 mL) and extracted with ethyl acetate (2×10 mL). The combined organic layers were dried over anhydrous sodium sulfate, concentrated, and purified by reversed phase flash chromatography [water (0.1% FA)/acetonitrile]. The desired fractions were collected, neutralized with solid NaHCO₃, concentrated, and extracted with ethyl acetate (2×20 mL). The combined organic layers were dried over anhydrous sodium sulfate and concentrated to afford the title compound (90.0 mg, 39% yield) as yellow solid; LCMS (ESI, M+1): m/z=383.1.

Step D. tert-butyl 2-((tert-butoxycarbonyl)(methyl)amino)-3-methoxy-7,8-dihydro-4H-pyrazolo[1,5-a][1,4]diazepine-5(6H)-carboxylate: To a solution of tert-butyl 2-[tert-butoxycarbonyl(methyl)amino]-3-hydroxy-4,6,7,8-tetrahydropyrazolo[1,5-a][1,4]diazepine-5-carboxylate (80.0 mg, 1.0 equiv) in THF (1 mL) was added NaH (16.7 mg, 60% purity, 2.0 equiv) at 0° C. The mixture was stirred at 0° C. for 0.5 hours, and then Mel (148 mg, 5.0 equiv) was added dropwise at 0° C. The resulting mixture was stirred at 20° C. for 2 hours. The reaction was quenched with water (15 mL) and extracted with EtOAc (5 mL×2). The combined organic layers were washed with brine (5 mL), dried over anhydrous sodium sulfate, filtered, concentrated, and purified by reversed phase flash chromatography [water (0.1% FA)/acetonitrile] to afford the title compound (70.0 mg, 83% yield) as yellow oil; LCMS (ESI, M+1): m/z=397.3.

Step E. 3-methoxy-N-methyl-5,6,7,8-tetrahydro-4H-pyrazolo[1,5-a][1,4]diazepin-2-amine: To a solution of tert-butyl 2-[tert-butoxycarbonyl(methyl)amino]-3-methoxy-4,6,7,8-tetrahydropyrazolo[1,5-a][1,4]diazepine-5-carboxylate (60.0 mg, 1.0 equiv) in MeCN (0.5 mL) was added HCl•dioxane (4 M, 1 mL). The reaction was stirred at 0° C. for 1 hour. Then the mixture was warmed to 20° C. and stirred for 2 hours. The mixture was concentrated to afford the title compound (35 mg, crude, HCl) as yellow solid.

The last two steps were performed according to Example 248. The title compound was obtained as off-white solid (FA salt). ¹H NMR (400 MHz, METHANOL-d₄) δ 7.50 (dd, J=5.6, 9.2 Hz, 1H), 7.13 (t, J=9.2 Hz, 1H), 6.98-6.93 (m, 2H), 5.40-5.20 (m, 1H), 4.85-4.66 (m, 2H), 4.23-3.96 (m, 7H), 3.70-3.62 (m, 4H), 3.52-3.44 (m, 1H), 3.43-3.33 (m, 4H), 3.30-3.23 (m, 2H), 3.18-3.02 (m, 2H), 2.79 (s, 3H), 2.71 (br d, J=15.0 Hz, 1H), 2.40-2.10 (m, 4H), 2.08-1.86 (m, 4H), 1.11 (t, J=7.2 Hz, 3H); LCMS (ESI, M+1): m/z=675.5.

### Example 394

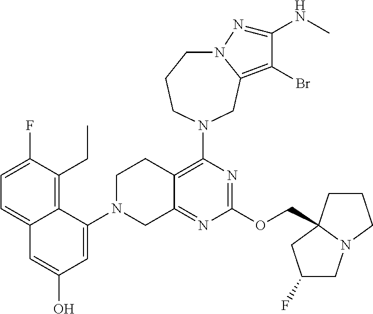 [SMILES: CCc1c(F)ccc2cc(O)cc(N3CCc4c(nc(OC[C@@]56CCCN5C[C@H](F)C6)nc4N4CCCn5nc(NC)c(Br)c5C4)C3)c12]

### 4-(4-(3-bromo-2-(methylamino)-7,8-dihydro-4H-pyrazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

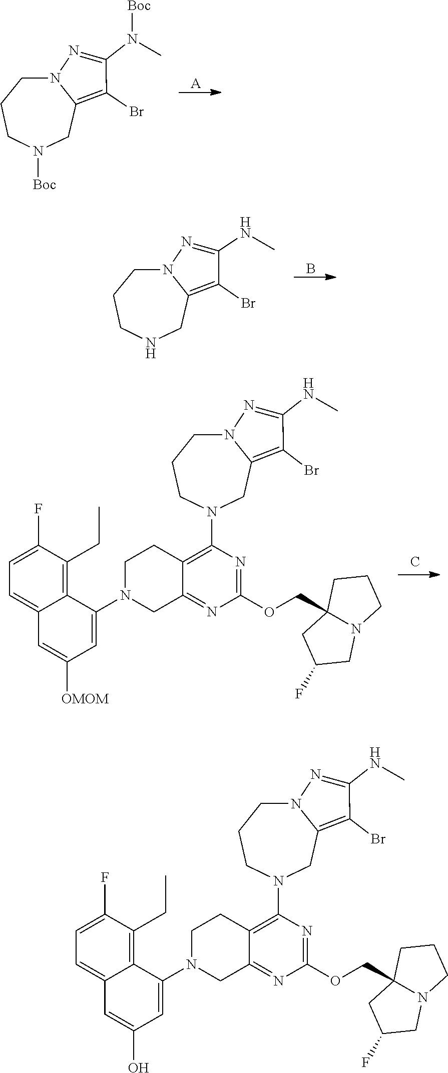

Step A. 3-bromo-N-methyl-5,6,7,8-tetrahydro-4H-pyrazolo[1,5-a][1,4]diazepin-2-amine: To a solution of tert-butyl 3-bromo-2-[tert-butoxycarbonyl(methyl)amino]-4,6,7,8-tetrahydropyrazolo[1,5-a][1,4]diazepine-5-carboxylate (150 mg, 1.0 equiv) in MeCN (0.5 mL) was added HCl•dioxane (4 M, 1 mL). The reaction was stirred at 0° C. for 1 hour. The reaction was concentrated to afford the title compound (80 mg, crude, HCl) as yellow solid.

The last two steps were performed according to Example 248. The title compound was obtained as off-white solid (FA salt). ¹H NMR (400 MHz, METHANOL-d₄) δ=7.50 (dd, J=5.6, 9.2 Hz, 1H), 7.14 (t, J=9.2 Hz, 1H), 7.00-6.93 (m, 2H), 5.40-5.21 (m, 1H), 4.79-4.63 (m, 2H), 4.31-4.22 (m, 1H), 4.22-4.04 (m, 4H), 4.03-3.86 (m, 2H), 3.66 (d, J=17.6 Hz, 1H), 3.48 (br d, J=10.4 Hz, 1H), 3.45-3.33 (m, 3H), 3.29-3.21 (m, 2H), 3.18-3.10 (m, 1H), 3.05 (dt, J=5.6, 9.6 Hz, 1H), 2.81 (s, 3H), 2.74-2.63 (m, 1H), 2.40-2.09 (m, 5H), 2.07-1.82 (m, 3H), 1.11 (t, J=7.2 Hz, 3H);LCMS (ESI, M+1): m/z=725.3.

### Example 395

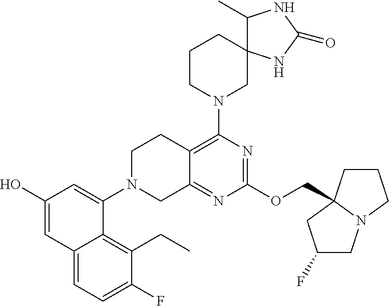 [SMILES: CCc1c(F)ccc2cc(O)cc(N3CCc4c(nc(OC[C@@]56CCCN5C[C@H](F)C6)nc4N4CCCC5(C4)NC(=O)NC5C)C3)c12]

### 7-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-4-methyl-1,3,7-triazaspiro[4.5]decan-2-one

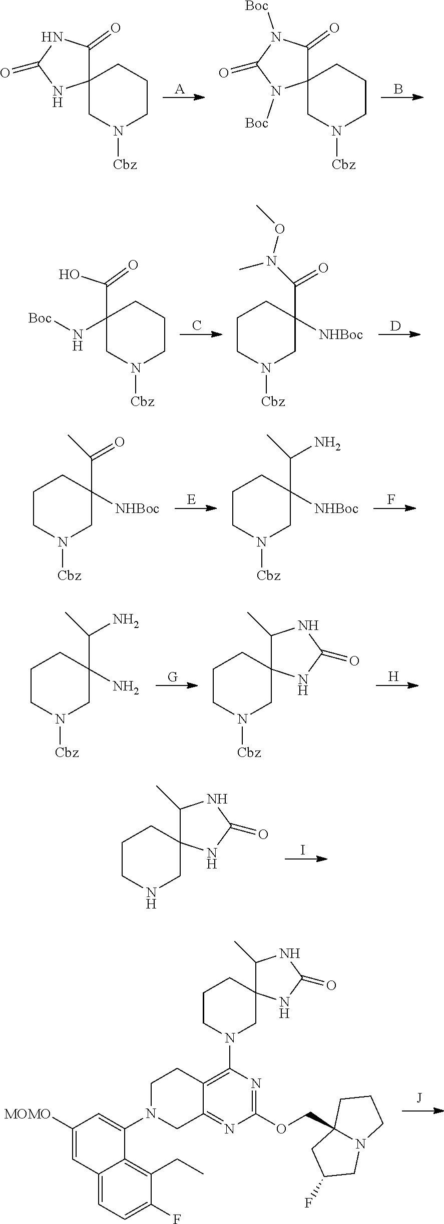

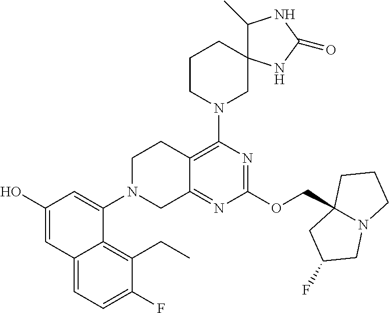 [SMILES: CCc1c(F)ccc2cc(O)cc(N3CCc4c(nc(OC[C@@]56CCCN5C[C@H](F)C6)nc4N4CCCC5(C4)NC(=O)NC5C)C3)c12]

Step A. 7-benzyl 1,3-di-tert-butyl 2,4-dioxo-1,3,7-triazaspiro[4.5]decane-1,3,7-tricarboxylate: A mixture of benzyl 2,4-dioxo-1,3,7-triazaspiro[4.5]decane-7-carboxylate (9 g, 1.0 equiv), (Boc)20 (25.9 g, 4.0 equiv), TEA (3.00 g, 1.0 equiv) and DMAP (93.0 mg, 0.02 equiv) in DME (300 mL) was stirred at 25° C. for 16 hours. The mixture was concentrated to give a yellow solid. The solid was dispersed in isopropyl ether (100 mL) and stirred for 10 minutes. The mixture was filtered and the solid was dried under reduced pressure to afford the title compound (11.8 g, crude) as white solid; LCMS (ESI, M-55): m/z=504.3.

Step B. 1-((benzyloxy)carbonyl)-3-((tert-butoxycarbonyllaminolpiperidine-3-carboxylic acid: To a solution of 7-benzyl 1,3-di-tert-butyl 2,4-dioxo-1,3,7-triazaspiro[4.5]decane-1,3,7-tricarboxylate (13.5 g, 1.0 equiv) in THF (135 mL) was added LiOH•H₂O (1 M in H20, 215 mL, 8.0 equiv) at 25° C. The mixture was stirred at 25° C. for 16 hours. The pH of the reaction was adjusted to 10 with 2N HCl below 5° C. (Boc)20 (20 mL) was added and the mixture was stirred at 25° C. for 60 hours. The mixture was extracted with MBTE (2×100 mL). The pH of the water phase was adjusted to 2˜3 with 2N HCl below 5° C. The mixture was extracted with ethyl acetate (4×100 mL). The combined organic layers were dried over anhydrous Na₂SO₄ and concentrated to afford the title compound (5.0 g, crude) as colorless gum; LCMS (ESI, M-55): m/z=323.1.

Step C. benzyl 3-((tert-butoxycarbonyl)amino)-3-(methoxy(methyl)carbamoyl)piperidine-1-carboxylate: To a solution of 1-((benzyloxy)carbonyl)-3-((tert-butoxycarbonyl)amino)piperidine-3-carboxylic acid (5.0 g, 1.0 equiv) and HATU (7.54 g, 1.5 equiv) in DMF (50 mL) were added N,0-dimethylhydroxylamine (1.61 g, 1.2 equiv, HCl) and DIPEA (8.54 g, 5.0 equiv). The mixture was stirred at 25° C. for 16 hours. The mixture was diluted with H₂O (400 mL) and extracted with ethyl acetate (4×50 mL). The combined organic layers were dried over anhydrous Na₂SO₄, concentrated, and purified with reversed phase flash chromatography [water (FA, 0.1%)/acetonitrile=3/7] to afford the title compound (2.4 g, 42% yield) as light yellow soild; LCMS (ESI, M+1): m/z=422.3.

Step D. benzyl 3-acetyl-3-((tert-butoxycarbonyl)amino)piperidine-1-carboxylate: To a solution of benzyl 3-((tert-butoxycarbonyl)amino)-3-(methoxy(methyl)carbamoyl)piperidine-1-carboxylate (2.2 g, 1.0 equiv) in THF (33 mL) was added MeMgBr (3 M, 4.35 mL, 2.5 equiv) dropwise at −10° C. The mixture was stirred between -10 and 15° C. for 6 hours. The mixture was quenched with saturated NH₄Cl aqueous (20 mL) and H₂O (40 mL) at 0° C. The mixture was extracted with ethyl acetate (4×30 mL). The combined organic layers was dried over anhydrous Na₂SO₄, concentrated, and purified with reversed phase flash chromatography [water (FA, 0.1%)/acetonitrile=1/1] to afford the title compound (0.48 g, 24% yield) as yellow solid; LCMS (ESI, M-55): m/z=321.2.

Step E. benzyl 3-(1-aminoethyl)-3-((tert-butoxycarbonyl)amino)piperidine-1-carboxylate: To a mixture of acetic acid;ammonia (1.38 g, 15 equiv) in MeOH (9 mL) was added benzyl 3-acetyl-3-((tert-butoxycarbonyl)amino)piperidine-1-carboxylate (0.45 g, 1.0 equiv) under N2 atmosphere. The mixture was stirred at 40° C. for 0.5 hours. NaBH3CN (113 mg, 1.5 equiv) was added, and the mixture was stirred at 40° C. for 110 hours. The mixture was quenched with saturated NaHCO₃ aqueous (10 mL) and stirred for 1 hour. The mixture was concentrated. The residue was diluted with H₂O (10 mL) and extracted with ethyl acetate (5×30 mL). The combined organic layers were dried over anhydrous Na₂SO₄, concentrated, and purified with reversed phase flash chromatography [water (FA, 0.1%)/acetonitrile=3/1] to afford the title compound (0.32 g, 70% yield,) as light-yellow gum; LCMS (ESI, M+1): m/z=378.3.

Step F. benzyl 3-amino-3-(1-aminoethyl)piperidine-1-carboxylate: To a solution of benzyl 3-(1-aminoethyl)-3-((tert-butoxycarbonyl)amino)piperidine-1-carboxylate (0.32 g, 1.0 equiv) in ACN (4 mL) was added HCl•dioxane (4 M, 8 mL) dropwise at 0° C. The mixture was stirred at 25° C. for 1 hour. The mixture was concentrated to afford the title compound (235 mg, 99% yield) as white solid.

Step G. benzyl 4-methyl-2-oxo-1,3,7-triazaspiro[4.5]decane-7-carboxylate: To a solution of benzyl 3-amino-3-(1-aminoethyl)piperidine-1-carboxylate (235 mg, 1.0 equiv) and DIPEA (816 mg, 7.5 equiv) in DMF (10 mL) was added CDI (165 mg, 1.2 equiv) in portions at 0° C. The mixture was stirred between 0 and 20° C. for 12 hours. The mixture was diluted with H₂O (150 mL) and extracted with ethyl acetate (4×20 mL). The combined organic layers were dried over anhydrous Na₂SO₄, concentrated, and purified with reversed phase flash chromatography [water (FA, 0.1%)/acetonitrile=3/2] to afford the tittle compound (0.2 g, 74% yield) as light-yellow gum; LCMS (ESI, M+1): m/z=304.2.

Step H. 4-methyl-1,3,7-triazaspiro[4.5]decan-2-one: Pd/C (40 mg, 10% purity) was added into MeOH (6 mL) under N2 atmosphere. Benzyl 4-methyl-2-oxo-1,3,7-triazaspiro[4.5]decane-7-carboxylate (0.2 g, 1.0 equiv) was added and the mixture was degassed and purged with H2 3 times. The mixture was stirred at 20° C. for 1 hour under H2 atmosphere (15 psi). The mixture was filtered through a pad of Celite. The filter cake was washed with DCM/MeOH=10/1 (20 mL). The combined organic phase was concentrated to afford the tittle compound (105 mg, 95% yield) as light-yellow gum.

The last two steps were performed according to Example 248. The title compound was obtained as white solid; ¹H NMR (400 MHz, methanol-d₄) δ=7.51 (br t, J=7.2 Hz, 1H), 7.14 (t, J=9.2 Hz, 1H), 6.99-6.96 (m, 2H), 5.26 (d, J=52.8 Hz, 1H), 4.37-3.93 (m, 4H), 3.80-3.58 (m, 3H), 3.55-3.35 (m, 7H), 3.28-2.88 (m, 4H), 2.80-2.73 (m, 1H), 2.47-1.76 (m, 10H), 1.23-1.15 (m, 3H), 1.10 (br t, J=7.2 Hz, 3H); LCMS (ESI, M+1): m/z=648.5.

### Example 396

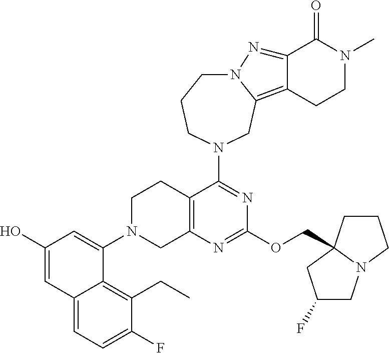 [SMILES: CCc1c(F)ccc2cc(O)cc(N3CCc4c(nc(OC[C@@]56CCCN5C[C@H](F)C6)nc4N4CCCn5nc6c(c5C4)CCN(C)C6=O)C3)c12]

### 2-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-9-methyl-2,3,4,5,10,11-hexahydro-1H-pyrido[3′,4′:3,4]pyrazolo[1,5-a][1,4]diazepin-8(9H)-one

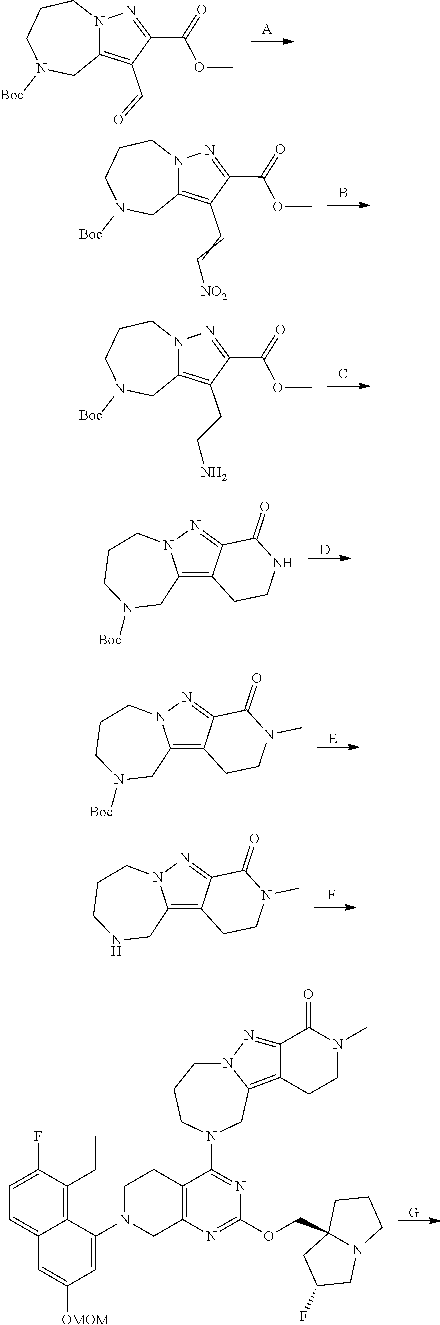

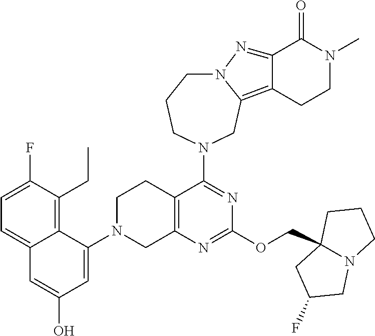 [SMILES: CCc1c(F)ccc2cc(O)cc(N3CCc4c(nc(OC[C@@]56CCCN5C[C@H](F)C6)nc4N4CCCn5nc6c(c5C4)CCN(C)C6=O)C3)c12]

Step A. 5-tert-butyl 2-methyl 3-(2-nitrovinyl)-7,8-dihydro-4H-pyrazolo[1,5-a][1,4]diazepine-2,5(6H)-dicarboxylate: To a solution of 5-tert-butyl 2-methyl 3-formyl-7,8-dihydro-4H-pyrazolo[1,5-a][1,4]diazepine-2,5(6H)-dicarboxylate (800 mg, 2 1.0 equiv) in AcOH (10 mL) were added nitromethane (8.15 g, 54 equiv) and NH40Ac (381 mg, 2.0 equiv). The mixture was stirred at 90° C. for 3 hours. The mixture was quenched with water (10 mL), neutralized with solid NaHCO3, and extracted with EtOAc (30 mL×2). The combined organic layers were washed with brine (30 mL), dried over anhydrous sodium sulfate, concentrated, and purified by reversed phase flash chromatography [water (0.1% FA)/acetonitrile]. The desired fractions were collected and neutralized with solid NaHCO₃ and concentrated under vacuum to remove acetonitrile. The aqueous layer was extracted with ethyl acetate (2×20 mL). The combined organic layers were dried over anhydrous sodium sulfate and concentrated under vacuum to afford the title compound (640 mg, 66% yield) as yellow solid; ¹H NMR (400 MHz, METHANOL-d₄) δ=4.45-4.33 (m, 4H), 3.71-3.61 (m, 2H), 3.47-3.36 (m, 2H), 2.71 (br t, J=6.6 Hz, 2H), 1.84-1.70 (m, 2H), 1.33 (br d, J=8.2 Hz, 9H); LCMS (ESI, M+l): m/z=367.2.

Step B. 5-tert-butyl 2-methyl 3-(2-aminoethyl)-7,8-dihydro-4H-pyrazolo[1,5-a][1,4]diazepine-2,5(6H)-dicarboxylate: To a solution of 05-tert-butyl 02-methyl 3-(2-nitrovinyl)-4,6,7,8-tetrahydropyrazolo[1,5-a][1,4]diazepine-2,5-dicarboxylate (100 mg, 273 umol, 1.0 equiv) in isopropanol (5 mL) were added HCl (12 M, 2.0 equiv) and Pd/C (20 mg, 10% purity) under N2 atmosphere. The suspension was degassed and purged with H2 3 times. The mixture was stirred under H2 (15 Psi) at 20° C. for 7 days. The mixture was filtered and the filtrate was concentrated and purified by reversed phase flash chromatography [water (0.10% FA)/acetonitrile]. The desired fractions were collected and neutralized with solid NaHCO₃ and concentrated under vacuum to remove acetonitrile. The mixture was extracted with ethyl acetate (2×10 mL). The combined organic layers were dried over anhydrous sodium sulfate and concentrated to afford the title compound (25 mg, 17.6% yield) as yellow solid; LCMS (ESI, M+1): m/z=339.2.

Step C. tert-butyl 8-oxo-4,5,8,9,10,11-hexahydro-1H-pyrido[3′,4′:3,4]pyrazolo[1,5-a][1,4]diazepine-2(3H)-carboxylate: To a solution of 05-tert-butyl 02-methyl 3-(2-aminoethyl)-4,6,7,8-tetrahydropyrazolo[1,5-a][1,4]diazepine-2,5-dicarboxylate (50 mg, 1.0 equiv) in MeOH (5 mL) was added N,N-diethylpropan-2-amine (77.2 L 3.0 equiv). The reaction was stirred at 80° C. for 16 hours. The mixture was concentrated and purified by reversed phase flash chromatography [water (0.1% FA)/acetonitrile]. The desired fractions were collected and neutralized with solid NaHCO₃ and concentrated under vacuum to remove acetonitrile. The aqueous layer was extracted with ethyl acetate (2×10 mL). The combined organic layers were dried over anhydrous sodium sulfate and concentrated under vacuum to afford the title compound (40 mg, 87% yield) as yellow solid; LCMS (ESI, M+1): m/z=307.2.

Step D. tert-butyl 9-methyl-8-oxo-4,5,8,9,10,11-hexahydro-1H-pyrido[3′,4′:3,4]pyrazolo[1,5-a][1,4]diazepine-2(3H)-carboxylate: To a solution of tert-butyl 6-oxo-5,8,9,13-tetrazatricyclo[7.5.0.02,7]tetradeca-1,7-diene-13-carboxylate (35 mg, 1.0 equiv) in THE (2 mL) was added NaH (9.14 mg, 60% purity, 2.0 equiv) at 0° C. After addition, the mixture was stirred at 0° C. for 0.5 hours, and then Mel (81.1 mg, 5.0 equiv) was added dropwise at 0° C. The resulting mixture was stirred at 20° C. for 16 hours. The mixture was quenched with water (1 mL) and extracted with ethyl acetate (2×1 mL). The combined organic layers were dried over anhydrous sodium sulfate, concentrated and purified by reversed phase flash chromatography [water (0.1% FA)/acetonitrile] to afford the title compound (30 mg, 82% yield) as yellow solid; LCMS (ESI, M+1): m/z=321.2.

Step E. 9-methyl-2,3,4,5,10,11-hexahydro-1H-pyrido[3′,4′:3,4]pyrazolo[1,5-a][1,4]diazepin-8(9H)-one: To a solution of tert-butyl 5-methyl-6-oxo-5,8,9,13-tetrazatricyclo[7.5.0.02,7]tetradeca-1,7-diene-13-carboxylate (30 mg, 1.0 equiv) in MeCN (0.1 mL) was added HCl•dioxane (4 M, 0.2 mL). The reaction was stirred at 0° C. for 1 hour. The mixture was concentrated under vacuum to afford the title compound (24 mg, crude, HCl) as yellow solid.

The last two steps were performed according to Example 248. The title compound was obtained white solid (FA salt). ¹H NMR (400 MHz, METHANOL-d₄) δ=7.57-7.48 (m, 1H), 7.19-7.11 (m, 1H), 6.91 (br d, J=4.4 Hz, 2H), 5.38-5.21 (m, 1H), 4.80-4.71 (m, 1H), 4.67-4.40 (m, 3H), 4.17-4.09 (m, 2H), 4.05 (br dd, J=3.6, 10.2 Hz, 3H), 3.72 (br d, J=18.0 Hz, 1H), 3.66-3.59 (m, 2H), 3.53-3.46 (m, 1H), 3.45-3.35 (m, 3H), 3.28-3.11 (m, 4H), 3.08 (s, 3H), 3.06-2.99 (m, 1H), 2.98-2.87 (m, 1H), 2.84-2.73 (m, 1H), 2.72-2.63 (m, 1H), 2.32-2.17 (m, 3H), 2.17-2.06 (m, 2H), 2.05-1.80 (m, 3H), 1.15-1.06 (m, 3H); LCMS (ESI, M+1): m/z=699.4.

### Example 397

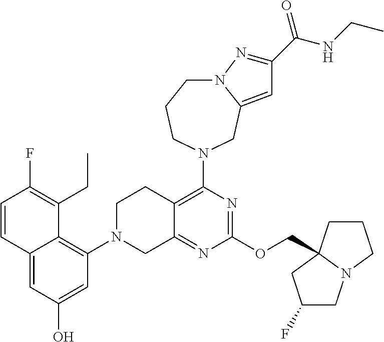 [SMILES: CCNC(=O)c1cc2n(n1)CCCN(c1nc(OC[C@@]34CCCN3C[C@H](F)C4)nc3c1CCN(c1cc(O)cc4ccc(F)c(CC)c14)C3)C2]

### N-ethyl-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepine-2-carboxamide

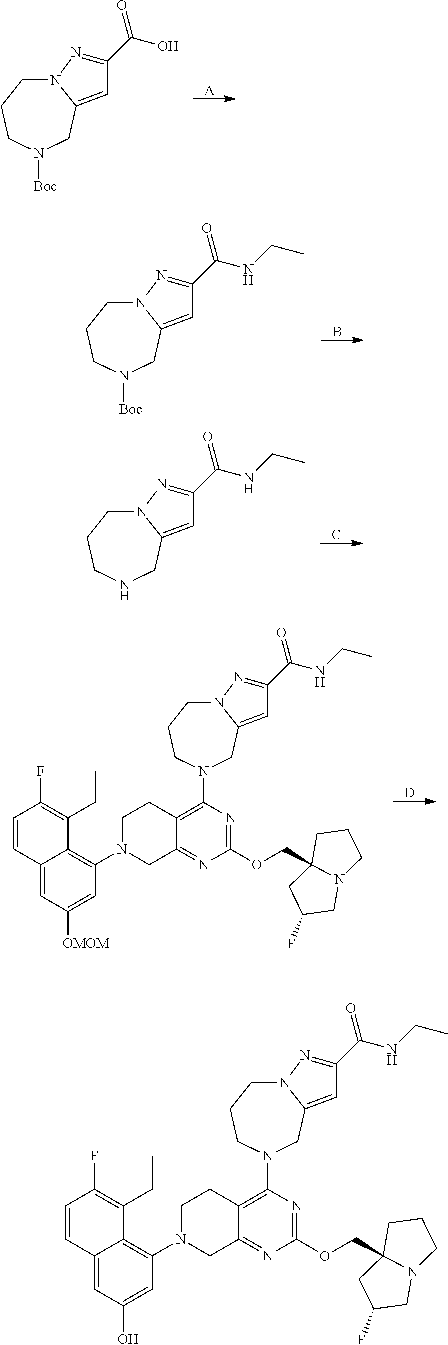

Synthesized according to Example 378. The title compound was obtained as white solid (FA salt). ¹H NMR (400 MHz, METHANOL-d₄) δ=7.51 (dd, J=5.6, 9.2 Hz, 1H), 7.14 (t, J=9.2 Hz, 1H), 7.01-6.93 (m, 2H), 6.69 (s, 1H), 5.47-5.19 (m, 1H), 5.02-4.93 (m, 1H), 4.81 (br d, J=3.2 Hz, 1H), 4.62-4.46 (m, 2H), 4.23-4.13 (m, 2H), 4.12-3.99 (m, 3H), 3.66 (d, J=17.6 Hz, 1H), 3.54 (br d, J=8.8 Hz, 1H), 3.41-3.32 (m, 6H), 3.28-3.12 (m, 3H), 3.07 (dt, J=5.6, 9.2 Hz, 1H), 2.73 (br d, J=14.4 Hz, 1H), 2.40-2.25 (m, 2H), 2.24-2.10 (m, 2H), 2.09-1.97 (m, 3H), 1.95-1.82 (m, 1H), 1.23-1.16 (m, 3H), 1.15-1.06 (m, 3H); LCMS (ESI, M+1): m/z=687.5.

### Example 398

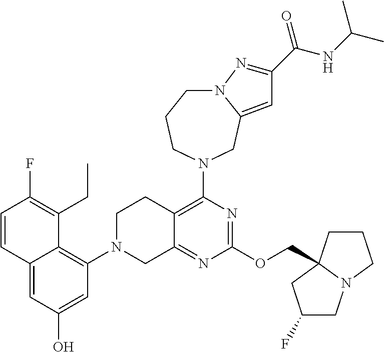 [SMILES: CCc1c(F)ccc2cc(O)cc(N3CCc4c(nc(OC[C@@]56CCCN5C[C@H](F)C6)nc4N4CCCn5nc(C(=O)NC(C)C)cc5C4)C3)c12]

### 3-bromo-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N-isopropyl-5,6,7,8-tetrahydro-4H-pyrazolo[1,5-a][1,4]diazepine-2-carboxamide

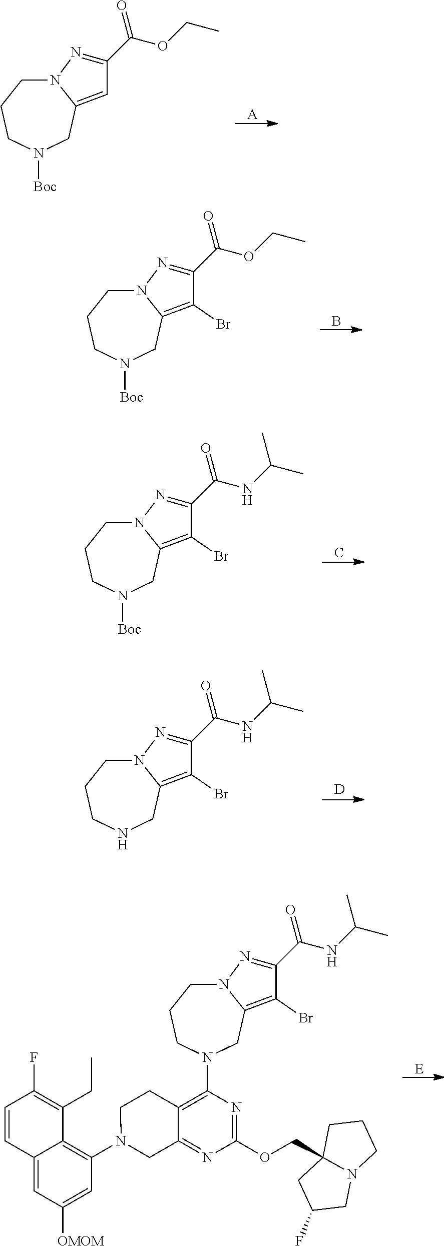

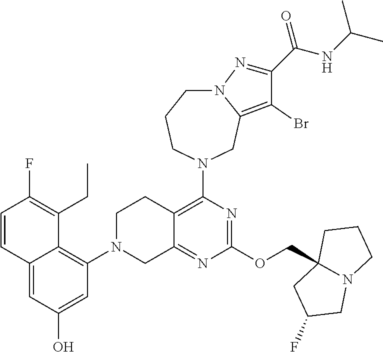 [SMILES: CCc1c(F)ccc2cc(O)cc(N3CCc4c(nc(OC[C@@]56CCCN5C[C@H](F)C6)nc4N4CCCn5nc(C(=O)NC(C)C)c(Br)c5C4)C3)c12]

Step A. 5-tert-butyl 2-ethyl 3-bromo-7,8-dihydro-4H-pyrazolo[1,5-a][1,4]diazepine-2,5(6H)-dicarboxylate: To a mixture of 5-tert-butyl 2-ethyl 7,8-dihydro-4H-pyrazolo[1,5-a][1,4]diazepine-2,5(6H)-dicarboxylate (200 mg, 1 equiv) in DMF (2 mL) was added NBS (173 mg, 1.5 equiv). The reaction was stirred at 60° C. for 1 hour under nitrogen atmosphere. The mixture was poured into H₂O (3 mL), extracted with ethyl acetate (3×3 mL), washed with brine (3×5 mL), dried over sodium sulfate, and concentrated to afford the title compound (130 mg, 50% yield); LCMS (ESI, M+1). m/z=389.8.

Step B. tert-butyl 3-bromo-2-(isopropylcarbamoyl)-7,8-dihydro-4H-pyrazolo[1,5-a][1,4]diazepine-5(6H)-carboxylate: To a solution of propan-2-amine (88.3 mg, 2 equiv) in toluene (2 mL) was added trimethylaluminum (2 M, 747 μL, 2 equiv) at 0° C. After stirring at 0° C. for 0.5 hours, 5-tert-butyl 2-ethyl 3-bromo-7,8-dihydro-4H-pyrazolo[1,5-a][1,4]diazepine-2,5(6H)-dicarboxylate (290 mg, 1 equiv) was added. The reaction was stirred at 80° C. for 2 hours. The mixture was diluted with saturated sodium sulfate aqueous solution (1.0 mL) and washed with THF (3×5.0 mL). The mixture was filtered, concentrated, and purified by reversed phase flash chromatography [C18, 0.1% formic acid condition] to afford the title compound (210 mg, 68% yield) as white solid; ¹H NMR (400 MHz, chloroform-d) δ=6.66-6.55 (m, 1H), 4.53 (br s, 2H), 4.45-4.37 (m, 2H), 4.26 (q, J=6.8, 8.0 Hz, 1H), 3.76 (br s, 2H), 1.99 (br s, 2H), 1.46 (s, 9H), 1.26 (s, 3H), 1.25 (s, 3H); LCMS (ESI, M+1): m/z=403.0.

Step C. 3-bromo-N-isopropyl-5,6,7,8-tetrahydro-4H-pyrazolo[1,5-a][1,4]diazepine-2-carboxamide: To a solution of tert-butyl 3-bromo-2-(isopropylcarbamoyl)-7,8-dihydro-4H-pyrazolo[1,5-a][1,4]diazepine-5(6H)-carboxylate (180 mg, 1 equiv) in ACN (0.9 mL) was added HCl•dioxane (4 M, 1.8 mL, 16 equiv) at 0° C. The reaction was stirred at 25° C. for 0.5 hours. The mixture was concentrated to afford the title compound (150 mg, crude, HCl) as white solid.

The last two steps were performed according to Example 248. The title compound was obtained as yellow solid. ¹H NMR (400 MHz,) δ=7.51 (dd, J=6.0, 9.2 Hz, 1H), 7.14 (t, J=9.6 Hz, 1H), 6.99-6.97 (m, 1H), 6.97-6.95 (m, 1H), 5.34--5.14 (m, 1H), 4.85-4.76 (m, 2H), 4.58-4.34 (m, 2H), 4.18-4.07 (m, 2H), 4.06-3.99 (m, 2H), 3.98-3.89 (m, 2H), 3.66 (br d, J=18.0 Hz, 1H), 3.53-3.47 (m, 1H), 3.42 (dq, J=2.8, 7.2 Hz, 2H), 3.26 (br d, J=3.2 Hz, 1H), 3.24-3.08 (m, 4H), 3.01-2.90 (m, 1H), 2.69 (br d, J=14.8 Hz, 1H), 2.44-2.32 (m, 1H), 2.31-2.15 (m, 2H), 2.15-2.01 (m, 2H), 1.97-1.86 (m, 2H), 1.86-1.75 (m, 1H), 1.23 (d, J=6.4 Hz, 6H), 1.11 (t, J=7.2 Hz, 3H); ¹⁹F NMR (400 MHz, chloroform-d) S=−123.059,-173.328; LCMS (ESI, M+1): m/z=781.2.

### Example 399

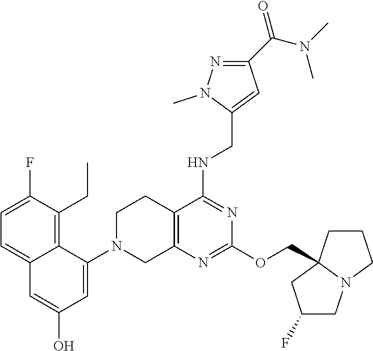 [SMILES: CCc1c(F)ccc2cc(O)cc(N3CCc4c(nc(OC[C@@]56CCCN5C[C@H](F)C6)nc4NCc4cc(C(=O)N(C)C)nn4C)C3)c12]

### 5-(((7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)amino)methyl)-N,N,1-trimethyl-1H-pyrazole-3-carboxamide

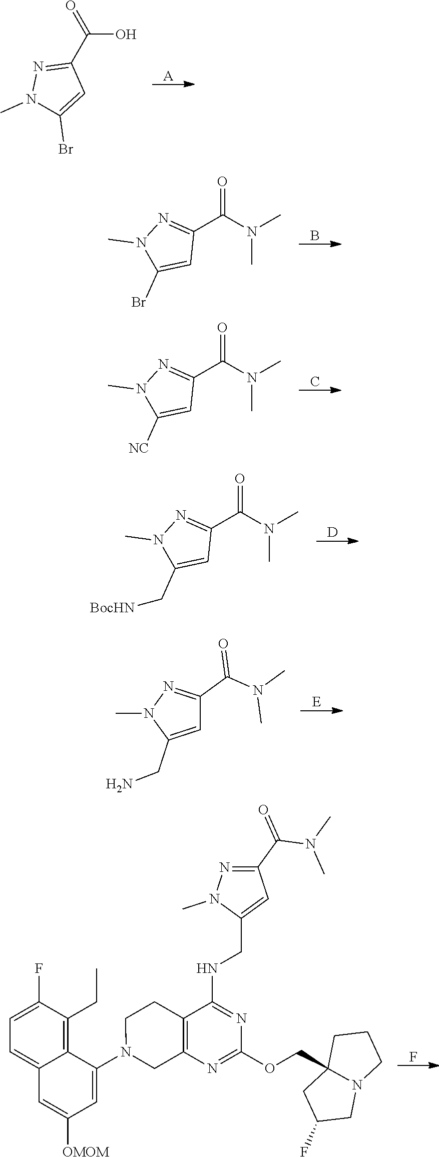

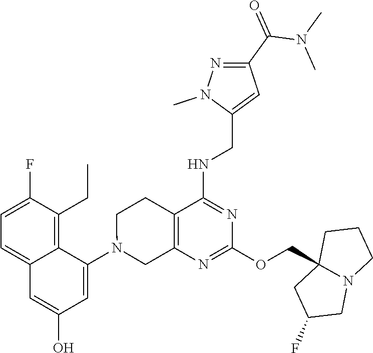 [SMILES: CCc1c(F)ccc2cc(O)cc(N3CCc4c(nc(OC[C@@]56CCCN5C[C@H](F)C6)nc4NCc4cc(C(=O)N(C)C)nn4C)C3)c12]

Step A. 5-bromo-N,N,1-trimethyl-1H-pyrazole-3-carboxamide: To a mixture of 5-bromo-1-methyl-1H-pyrazole-3-carboxylic acid (5.00 g, 1.0 equiv) in THF (50 mL) was added dimethylamine (2 M in THF, 24.4 mL, 2.0 equiv) and DIEA (15.8 g, 5.0 equiv) at 0° C. HATU (18.5 g, 2.0 equiv) was added. The reaction was stirred at 25° C. for 0.5 hours. The mixture was diluted with water (100 mL) and extracted with ethyl acetate (3×50 mL). The combined organic layers were washed with brine (30 mL), dried over anhydrous sodium sulfate, concentrated, and purified by column chromatography (SiO2, petroleum ether/ethyl acetate=I/O to 2/1) to afford the title compound (4.10 g, 72% yield) as yellow solid; LCMS (ESI, M+1, M+3): m/z=232.0, 234.0.

Step B. 5-cyano-N,N,1-trimethyl-1H-pyrazole-3-carboxamide: To a mixture of 5-bromo-N,N,1-trimethyl-1H-pyrazole-3-carboxamide (250 mg, 1.0 equiv) in DMF (2.5 mL) was added CuCN (193 mg, 2.0 equiv). The mixture was degassed and purged with nitrogen 3 times. The reaction was stirred at 150° C. for 4 hours under nitrogen atmosphere. The mixture was filtered, diluted with water (10 mL), and extracted with EtOAc (3×10 mL). The combined organic layers were dried, filtered, concentrated, and purified by silica gel chromatography [petroleum ether/ethyl acetate 5/1 to 1/1] to afford the title compound (90 mg, 40% yield) as white solid; ¹H NMR (400 MHz, chloroform -d) δ=7.23-7.19 (m, 1H), 4.13-4.06 (m, 3H), 3.34 (s, 3H), 3.12 (s, 3H); LCMS (ESI, M+1): m/z=179.1.

Step C. tert-butyl ((3-(dimethylcarbamoyl)-1-methyl-1H-pyrazol-5-yl)methyl)carbamate: To a mixture of 5-cyano-N,N,1-trimethyl-1H-pyrazole-3-carboxamide (80 mg, 1.0 equiv) in methanol (4 mL) were added Boc20 (294 mg, 3.0 equiv) and Pd/C (20 mg, 10% purity). The reaction was degassed and purged with hydrogen several times. The reaction was stirred at 20° C. for 14 hours under hydrogen (15 psi) atmosphere. The mixture was filtered, concentrated, and purified by prep-TLC [ethyl acetate/methanol 10/1] to afford the title compound (50 mg, 38% yield) as white solid; ¹H NMR (400 MHz, chloroform -d) δ=6.57 (s, 1H), 4.40-4.33 (m, 2H), 3.90-3.85 (m, 3H), 3.10 (br s, 6H), 1.48-1.43 (m, 9H); LCMS (ESI, M+1): m/z=283.0.

Step D. 5-(aminomethyl)-N,N,1-trimethyl-1H-pyrazole-3-carboxamide: To a mixture of tert-butyl ((3-(dimethylcarbamoyl)-1-methyl-1H-pyrazol-5-yl)methyl)carbamate (85 mg, 1.0 equiv) in ACN (0.6 mL) was added HCl•dioxane (4 M, 1.13 mL, 15 equiv). The reaction was stirred at 20° C. for 0.5 hours. The mixture was concentrated, dissolved in methanol (1 mL), neutralized with solid NaHCO₃, filtered, and concentrated to afford the title compound (50 mg, 91% yield) as white solid.

The last two steps were performed according to Example 248 except that K3P04 instead of N,N-diethylpropan-2-amine was used in step D. The title compound was obtained as pink solid (FA salt). ¹H NMR (400 MHz, methanol-d₄) δ=7.54-7.47 (m, 1H), 7.17-7.10 (m, 1H), 7.04-6.94 (m, 2H), 6.59-6.55 (m, 1H), 5.48-5.30 (m, 1H), 4.78 (dd, J=4.0, 18.4 Hz, 2H), 4.30-4.16 (m, 2H), 3.98-3.96 (m, 3H), 3.94-3.87 (m, 1H), 3.71-3.64 (m, 1H), 3.58-3.44 (m, 4H), 3.35-3.33 (m, 5H), 3.19-3.11 (m, 1H), 3.10-3.06 (m, 3H), 2.88-2.78 (m, 1H), 2.60-2.51 (m, 1H), 2.19 (br d, J=11.2 Hz, 4H), 2.15-2.06 (m, 2H), 2.03-1.90 (m, 1H), 1.07-1.01 (m, 3H); ¹⁹F NMR (400 MHz, methanol-d₄) δ=−123.116, -173.814; LCMS (ESI, M+1): m/z=661.1.

### Example 400

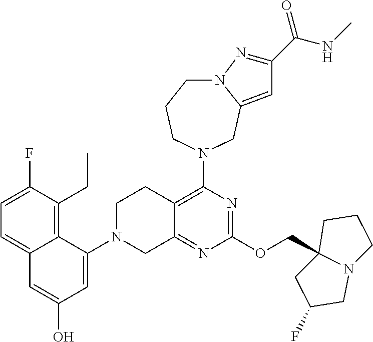 [SMILES: CCc1c(F)ccc2cc(O)cc(N3CCc4c(nc(OC[C@@]56CCCN5C[C@H](F)C6)nc4N4CCCn5nc(C(=O)NC)cc5C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepine-2-carboxamide

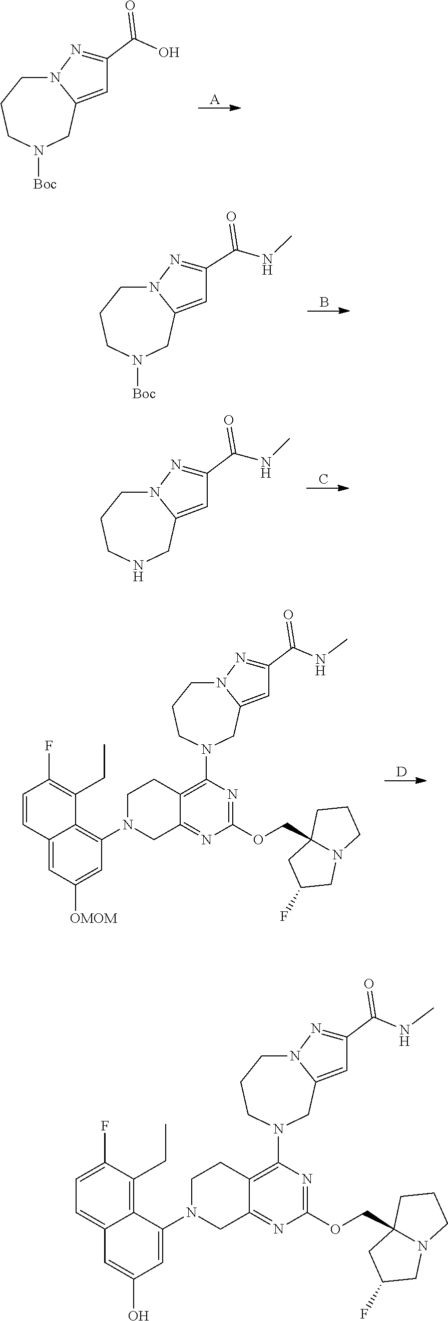

Step A. tert-butyl 2-(methylcarbamoyl)-7,8-dihydro-4H-pyrazolo[1,5-a][1,4]diazepine-5(6H)-carboxylate: To a solution of 5-(tert-butoxycarbonyl)-5,6,7,8-tetrahydro-4H-pyrazolo[1,5-a][1,4]diazepine-2-carboxylic acid (300 mg, 1.0 equiv) and methanamine (288 mg, 4.0 equiv, HCl) in DMF (3.0 mL) were added HATU (811 mg, 2.0 equiv) and N,N-diethylpropan-2-amine (1.38 g, 10 equiv). The reaction was stirred at 25° C. for 0.5 hours. The reaction was filtered and purified by reversed phase HPLC (0.1% FA condition) to afford the title compound (179 mg, 57% yield) as yellow oil; LCMS (ESI, M+1): m/z=294.9.

Step B. N-methyl-5,6,7,8-tetrahydro-4H-pyrazolo[1,5-a][1,4]diazepine-2-carboxamide: To a solution of tert-butyl 2-(methylcarbamoyl)-7,8-dihydro-4H-pyrazolo[1,5-a][1,4]diazepine-5(6H)-carboxylate (175 mg, 1.0 equiv) in MeCN (2.0 mL) was added HCl/dioxane (4 M, 0.8 mL, 5.1 equiv). The reaction was stirred at 25° C. for 0.5 hours. The mixture was concentrated under vacuum to afford the title compound (140 mg, crude) as white solid.

The last two steps were performed according to Example 248. The title compound was obtained as yellow solid (FA salt). ¹H NMR (400 MHz, METHANOL-d₄) δ=7.55-7.45 (m, 1H), 7.17-7.10 (m, 1H), 7.03-6.93 (m, 2H), 6.69 (d, J=1.6 Hz, 1H), 5.49-5.31 (m, 1H), 4.99-4.84 (m, 2H), 4.59-4.47 (m, 2H), 4.31-4.23 (m, 1H), 4.22-4.00 (m, 4H), 3.71-3.64 (m, 1H), 3.59-3.43 (m, 4H), 3.43-3.35 (m, 2H), 3.25-3.12 (m, 3H), 2.88 (s, 3H), 2.73 (br d, J=14.4 Hz, 1H), 2.51-2.21 (m, 4H), 2.17-2.09 (m, 2H), 2.08-1.93 (m, 2H), 1.16-1.05 (m, 3H); LCMS (ESI, M+1): m/z =673.6.

### Example 401

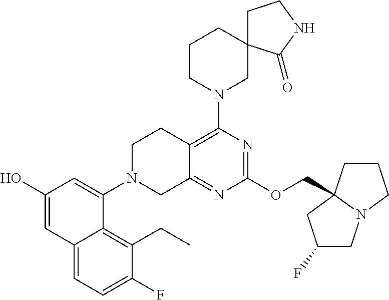 [SMILES: CCc1c(F)ccc2cc(O)cc(N3CCc4c(nc(OC[C@@]56CCCN5C[C@H](F)C6)nc4N4CCCC5(CCNC5=O)C4)C3)c12]

### 7-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-2,7-diazaspiro[4.5]decan-1-one

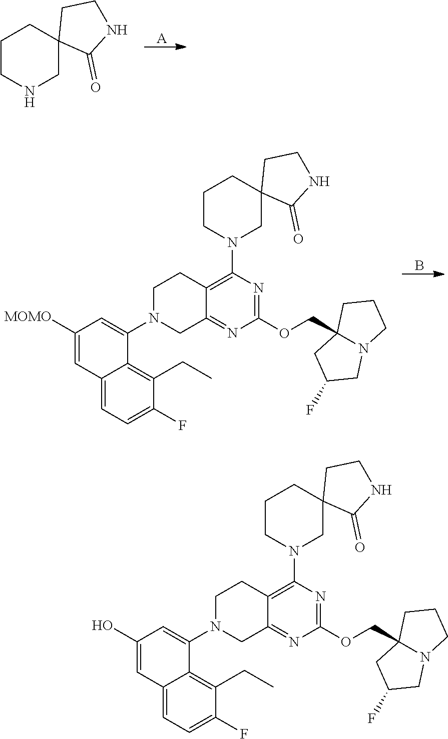

Synthesized according to Example 248. The title compound was obtained as yellow solid (FA salt). ¹H NMR (400 MHz, METHANOL-d₄) δ=7.57-7.44 (m, 1H), 7.20-7.10 (m, 1H), 7.04-6.88 (m, 2H), 5.48-5.25 (m, 1H), 4.34-4.11 (m, 3H), 4.09 (s, 2H), 3.67-3.34 (m, 8H), 3.26-3.09 (m, 4H), 3.05-2.93 (m, 1H), 2.73-2.59 (m, 1H), 2.47-1.63 (m, 13H), 1.15-1.06 (m, 3H); LCMS (ESI, M+1): m/z=633.7.

### Example 402

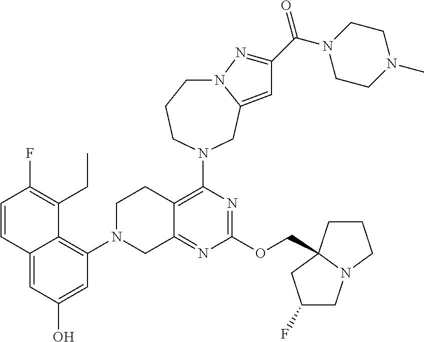 [SMILES: CCc1c(F)ccc2cc(O)cc(N3CCc4c(nc(OC[C@@]56CCCN5C[C@H](F)C6)nc4N4CCCn5nc(C(=O)N6CCN(C)CC6)cc5C4)C3)c12]

### (5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(4-methylpiperazin-1-yl)methanone

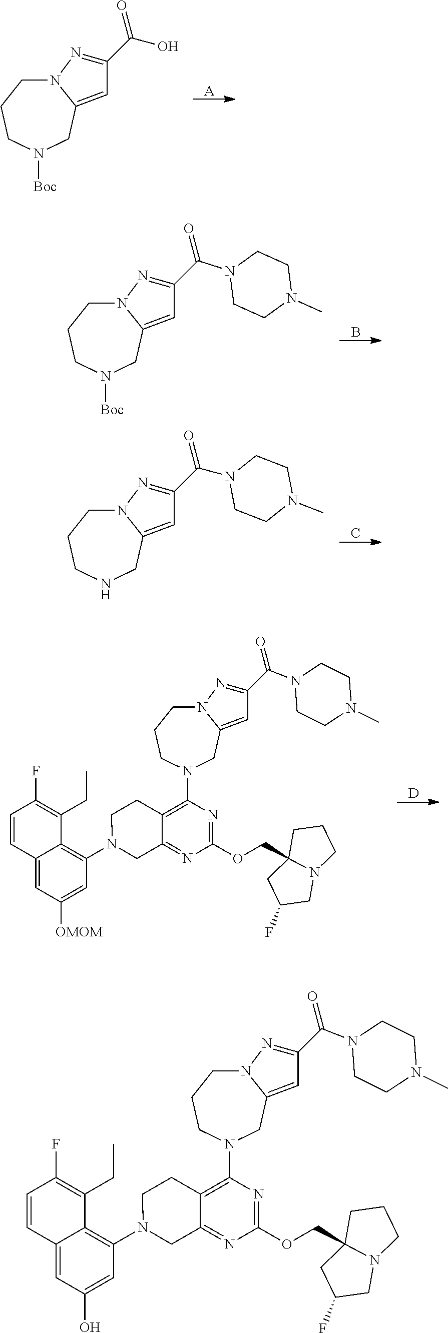

Step A. tert-butyl 2-(4-methylpiperazine-1-carbonyl)-7,8-dihydro-4H-pyrazolo[1,5-a][1,4]diazepine-5(6H)-carboxylate: To a solution of 5-(tert-butoxycarbonyl)-5,6,7,8-tetrahydro-4H-pyrazolo[1,5-a][1,4]diazepine-2-carboxylic acid (7.00 g, 1.0 equiv) in DMF (70 mL) were added HATU (28.4 g, 3.0 equiv), N,N-diethylpropan-2-amine (19.3 g, 6 equiv) and methylcyclohexane (4.98 g, 2.0 equiv). The reaction was stirred at 25° C. for 2 hours. The mixture was diluted with H₂O (50 mL) and extracted with EtOAc (3×50 mL). The combined organic layers were washed with brine (2×50 mL), dried over anhydrous sodium sulfate, concentrated, and purified by reversed phase HPLC (0.1% FA condition) to afford the title compound (7.00 g, 77% yield) as white solid; LCMS (ESI, M+1): m/z=364.2.

Step B. (4-methylpiperazin-1-yl)(5,6,7,8-tetrahydro-4H-pyrazolo[1,5-a][1,4]diazepin-2-yl)methanone: To a solution of tert-butyl 2-(4-methylpiperazine-1-carbonyl)-7,8-dihydro-4H-pyrazolo[1,5-a][1,4]diazepine-5(6H)-carboxylate (7.00 g, 1.0 equiv) in MeCN (10 mL) was added HCl•dioxane (4 M, 70 mL, 15 equiv). The reaction was stirred at 20° C. for 0.5 hours. The reaction was concentrated under reduced pressure to afford the title compound (5.50 g, crude, HCl) as white solid; LCMS (ESI, M+1): m/z=264.0. The last two steps were performed according to Example 248. The title compound was obtained as white solid; ¹H NMR (400 MHz, METHANOL-d₄) δ=7.51 (dd, J=5.6, 8.8 Hz, 1H), 7.14 (t, J=9.6 Hz, 1H), 6.96 (s, 2H), 6.60 (d, J=1.2 Hz, 1H), 5.36-5.16 (m, 1H), 5.01 (br d, J=16.4 Hz, 1H), 4.84-4.79 (m, 1H), 4.60-4.47 (m, 2H), 4.29-4.17 (m, 1H), 4.12-3.93 (m, 6H), 3.76 (br s, 2H), 3.66 (br d, J=17.6 Hz, 1H), 3.57-3.48 (m, 1H), 3.46-3.34 (m, 2H), 3.28-3.12 (m, 5H), 2.98 (dt, J =5.6, 9.2 Hz, 1H), 2.71 (br d, J=14.0 Hz, 1H), 2.48 (br d, J=13.6 Hz, 4H), 2.32 (s, 3H), 2.30-2.23 (m, 1H), 2.21-1.77 (m, 7H), 1.10 (dt, J=1.2, 7.2 Hz, 3H); LCMS (ESI, M+1): m/z=742.4.

### Example 403

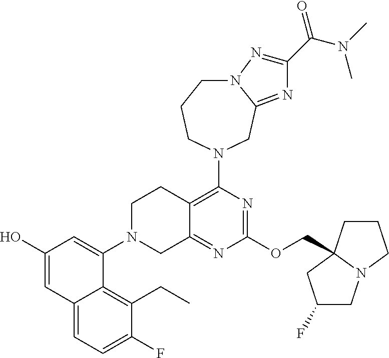 [SMILES: CCc1c(F)ccc2cc(O)cc(N3CCc4c(nc(OC[C@@]56CCCN5C[C@H](F)C6)nc4N4CCCn5nc(C(=O)N(C)C)nc5C4)C3)c12]

### 8-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-6,7,8,9-tetrahydro-5H-[1,2,4]triazolo[1,5-a][1,4]diazepine-2-carboxamide

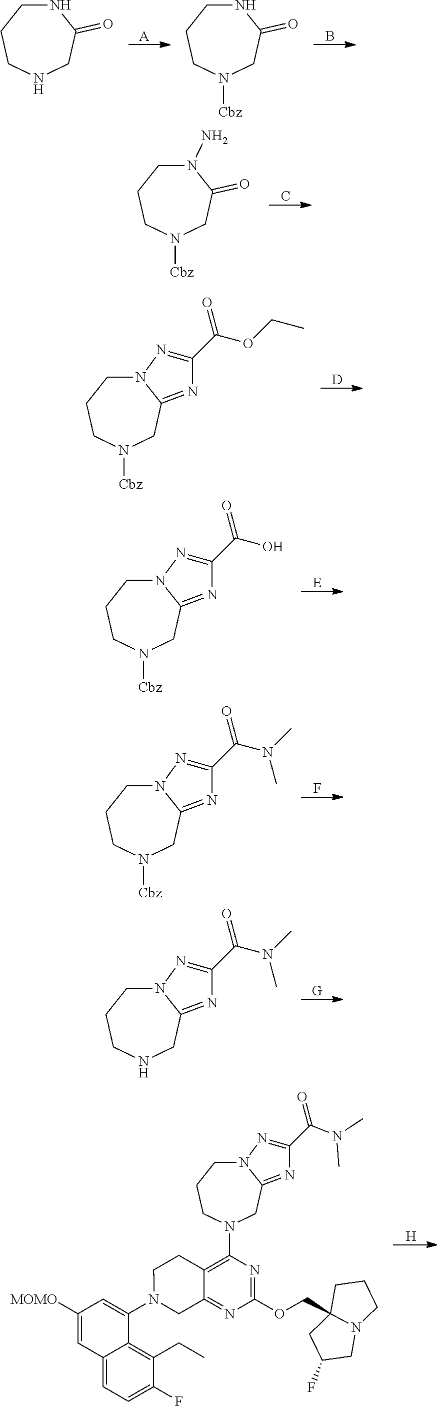

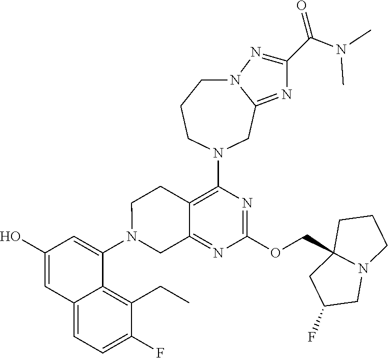 [SMILES: CCc1c(F)ccc2cc(O)cc(N3CCc4c(nc(OC[C@@]56CCCN5C[C@H](F)C6)nc4N4CCCn5nc(C(=O)N(C)C)nc5C4)C3)c12]

Step A. benzyl 3-oxo-1,4-diazepane-1-carboxylate: A mixture of 1,4-diazepan-2-one (18 g, 1 equiv, HCl) and NaHCO₃ (25 g, 2.5 equiv) in H₂O (150 mL) and THF (150 mL) was stirred at 20° C. for 0.1 hours. CbzCl (21.4 g, 1.05 equiv) was added dropwise, and the mixture was stirred at 20° C. for 12 hours. The reaction was extracted with ethyl acetate (3×250 mL) at 20° C., dried over NaSO₄, and concentrated. The residue was dispersed in [petroleum ether/ethyl acetate=l/1 (300 mL)] and stirred for 0.5 hours. The mixture was filtered and concentrated to afford the title compound (20 g, 60% yield) as white powder; 1H NMR (400 MHz, METHANOL-d₄) δ=7.44-7.20 (m, 5H), 5.15 (s, 2H), 4.14 (s, 2H), 3.67 (br d, J=4.8 Hz, 2H), 1.81 (br d, J=3.6 Hz, 2H); LCMS (ESI, M+1): m/z=249.2.

Step B. benzyl 4-amino-3-oxo-1,4-diazepane-1-carboxylate: A solution of benzyl 3-oxo-1,4-diazepane-1-carboxylate (27.5 g, 1.0 equiv) in DMF (250 mL) was cooled to 0° C. and the mixture was stirred for 20 minutes at 0° C. Then to the mixture were added t-BuOK (1 M, 277 mL, 2.5 equiv) and amino hydrogen sulfate (25 g, 2.0 equiv) at 0° C. The mixture was stirred at 0° C. for 20 minutes. The reaction was quenched with water (30 mL) at 0° C., extracted with ethyl acetate (3×50 mL), dried over NaSO₄, and concentrated to afford the title compound (30 g, crude) as yellow oil; LCMS (ESI, M+1): m/z=264.3

Step C. 8-benzyl 2-ethyl 6,7-dihydro-5H-[1,2,4]triazolo[1,5-ai[1,4]diazepine-2,8(9H)-dicarboxylate: To a solution of benzyl 4-amino-3-oxo-1,4-diazepane-1-carboxylate (30 g, 50% purity, 1.0 equiv) in EtOH (220 mL) were added ethyl 2-ethoxy-2-imino-acetate (16.5 g, 2.0 equiv) and AcOH (12 g, 3.5 equiv) at 23° C. Then the mixture was stirred at 78° C. for 18 hours. The reaction was concentrated, diluted with ethyl acetate (50 mL) and water (80 mL), extracted with ethyl acetate (6×40 mL), and dried over Na₂SO₄. The combined organic layers were concentrated and purified by reversed phase flash chromatography {0.1% FA condition] to afford the title compound (5.1 g, 20% yield) as yellow oil; LCMS (ESI, M+1): m/z=345.1

Step D. 8-((benzyloxy)carbonyl)-6,7,8,9-tetrahydro-5H-[1,2,4]triazolo[1,5-a][1,4]diazepine-2-carboxylic acid: To a solution of 8-benzyl 2-ethyl 6,7-dihydro-5H-[1,2,4]triazolo[1,5-a][1,4]diazepine-2,8(9H)-dicarboxylate (3.1 g, 1.0 equiv) in ETOH (20 mL) was added NaOH (1 M, 18.00 mL, 2.0 equiv) at 0° C. The mixture was stirred at 25° C. for 1 hour. The mixture was poured into HCl solution (40 mL) and filtered. The filtrate was extracted with ethyl acetate (2×20 mL), dried over Na₂SO₄, and concentrated to afford the title compound (2.8 g, 95% yield) as white foam; LCMS (ESI, M+1): m/z=317.1

Step E. benzyl 2-(dimethylcarbamoyl)-6,7-dihydro-5H-[1,2,4]triazolo[1,5-a][1,41diazepine-8(9H)-carboxylate: To a solution of 8-((benzyloxy)carbonyl)-6,7,8,9-tetrahydro-5H-[1,2,4]triazolo[1,5-a][1,4]diazepine-2-carboxylic acid (4 g, 1.0 equiv) in DMF (5 mL) were added EDCI (4.84 g, 2 equiv), HOBt (2.4 g, 1.2 equiv), TEA (10 g, 8.0 equiv) and N-methylmethanamine (2 M, 3.0 mL, 5.0 equiv). The mixture was stirred at 40° C. for 24 hours. The mixture was filtered and purified by reversed phase flash chromatography {0.1% FA condition] to afford the title compound (1.2 g, 30% yield) as brown oil; LCMS (ESI, M+1): m/z=344.2

Step F. N,N-dimethyl-6,7,8,9-tetrahydro-5H-[1,2,4]triazolo[1,5-a][1,4]diazepine-2-carboxamide: Pd/C (200 mg, 10% purity, 1.0 equiv) was added into MeOH (10 mL) under N2 atmosphere. Benzyl 2-(dimethylcarbamoyl)-6,7-dihydro-5H-[1,2,4]triazolo[1,5-a][1,4]diazepine-8(9H)-carboxylate (1.2 g, 1.0 equiv) in MeOH (10 mL) was added and the mixture was degassed and purged with H2 3 times. The mixture was stirred at 20° C. for 1 hour under H2 atmosphere (15 psi). The mixture was filtered through a pad of Celite. The organic phase was concentrated to afford the title compound (700 mg, 96% yield) as white solid; LCMS (ESI, M+1): m/z=210.1

The last two steps were performed according to Example 248. The title compound was obtained as yellow solid; 1H NMR (400 MHz, METHANOL-d₄) δ=7.60 (dd, J=6.0, 8.8 Hz, 1H), 7.13 (t, J=9.2 Hz, 1H), 7.04-6.90 (m, 2H), 5.34-5.15 (m, 1H), 5.06-4.98 (m, 1H), 4.95-4.88 (m, 1H), 4.55-4.46 (m, 1H), 4.44-4.35 (m, 1H), 4.17-3.86 (m, 5H), 3.66 (br d, J=17.6 Hz, 1H), 3.50 (br d, J=9.2 Hz, 1H), 3.41-3.33 (m, 2H), 3.29-3.05 (m, 8H), 3.01-2.92 (m, 1H), 2.70 (br d, J=14.8 Hz, 1H), 2.42-2.31 (m, 1H), 2.29-2.02 (m, 4H), 1.97-1.78 (m, 3H), 1.14-1.03 (m, 3H); LCMS (ESI, M+1): m/z=688.2.

### Example 404

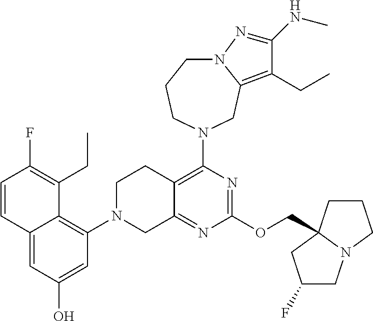 [SMILES: CCc1c(NC)nn2c1CN(c1nc(OC[C@@]34CCCN3C[C@H](F)C4)nc3c1CCN(c1cc(O)cc4ccc(F)c(CC)c14)C3)CCC2]

### 5-ethyl-4-(4-(3-ethyl-2-(methylamino)-7,8-dihydro-4H-pyrazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyridin-7(8H)-yl)-6-fluoronaphthalen-2-ol

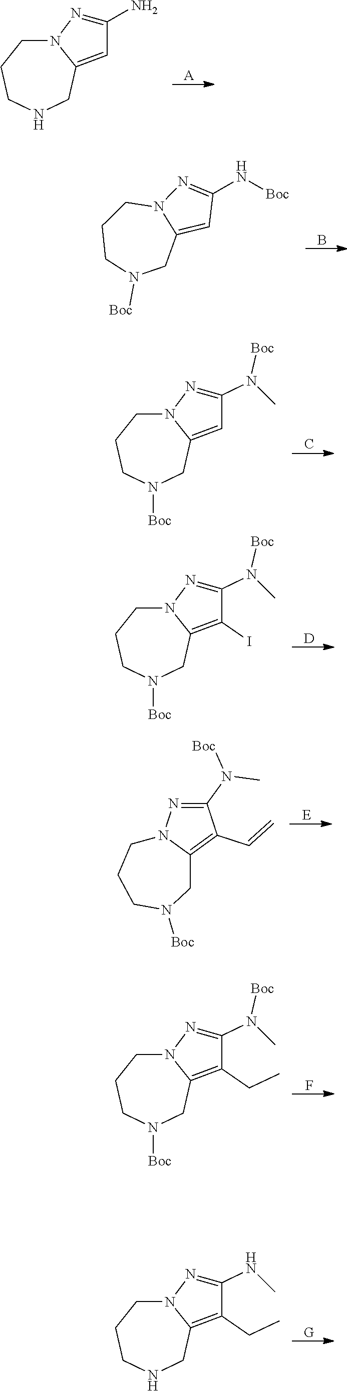

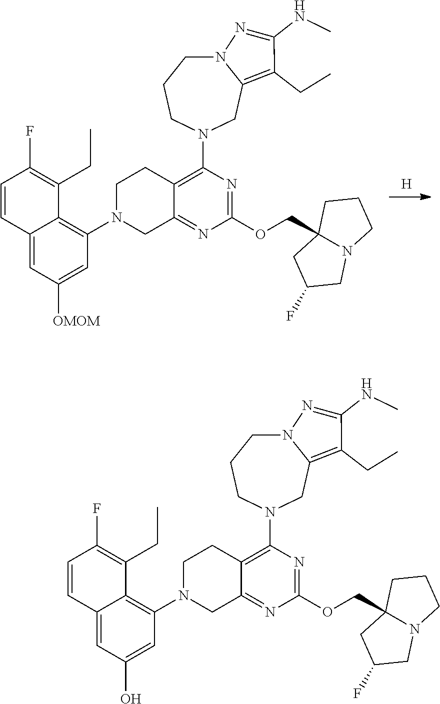

Step A. tert-butyl 2-((tert-butoxycarbonyl)amino)-7,8-dihydro-4H-pyrazolo[1,5-a][1,4]diazepine-5(6H)-carboxylate: A solution of 5,6,7,8-tetrahydro-4H-pyrazolo[1,5-a][1,4]diazepin-2-amine (2.00 g, 1.0 equiv) in (Boc)20 (20 mL) was stirred at 60° C. for 2 hours. The mixture was diluted with water (30 mL) and extracted with EtOAc (3×20 mL). The combined organic layers were dried over anhydrous sodium sulfate, concentrated, and purified by column chromatography [SiO₂, Petroleum ether/Ethyl acetate=100/1 to 0/1]. The crude product was dissolved in MeOH and stirred with K2CO₃ at 40° C. for 4 hours. The mixture was filtered and concentrated. Then the residue was triturated with H₂O (40 mL) at 25° C. for 10 minutes and filtered to afford the title compound (4.70 g, 92% yield) as white solid; LCMS (ESI, M-55): m/z=297.2.

Step B. tert-butyl 2-((tert-butoxycarbonyl)(methyl)amino)-7,8-dihydro-4H-pyrazolo[1,5-a][1,4]diazepine-5(6H)-carboxylate: To a solution of tert-butyl 2-((tert-butoxycarbonyl)amino)-7,8-dihydro-4H-pyrazolo[1,5-a][1,4]diazepine-5(6H)-carboxylate (3.50 g, 1.0 equiv) in THF (34 mL) was added NaH (794 mg, 60% purity, 2.0 equiv) at 0° C.. After addition, the mixture was stirred at 25° C. for 1 hour, and then Mel (7.05 g, 5.0 equiv) was added dropwise at 0° C. The resulting mixture was stirred at 25° C. for 1 hour. The mixture was diluted with water (20 mL) and extracted with EtOAc (3×30 mL). The combined organic layers were dried over anhydrous sodium sulfate, concentrated, and purified by column chromatography [SiO₂, Petroleum ether/Ethyl acetate=10/1 to 3/1] to afford the title compound (3.20 g, 87% yield) as white solid; ¹H NMR (400 MHz, CHLOROFORM-d) δ=6.77-5.82 (m, 1H), 4.50-4.33 (m, 2H), 4.32-4.23 (m, 2H), 3.68 (br s, 2H), 3.30 (s, 3H), 1.99-1.85 (m, 2H), 1.52 (s, 9H), 1.43 (s, 9H); LCMS (ESI, M+1): m/z=367.1.

Step C. tert-butyl 2-((tert-butoxycarbonyl)(methyl)amino)-3-iodo-7,8-dihydro-4H-pyrazolo[1,5-a][1,4]diazepine-5(6H)-carboxylate: To a solution of tert-butyl 2-((tert-butoxycarbonyl)(methyl)amino)-7,8-dihydro-4H-pyrazolo[1,5-a][1,4]diazepine-5(6H)-carboxylate (500 mg, 1.0 equiv) in AcOH (2.5 mL) was added NIS (614 mg, 2.0 equiv).The mixture was stirred at 80° C. for 1 hour. The mixture was filtered, washed with MeCN (1 mL), and purified by reversed phase flash chromatography [C18, 0.1% formic acid condition] to afford the title compound (550 mg, 81% yield) as yellow solid; LCMS (ESI, M-55): m/z=437.1.

Step D. tert-butyl 2-((tert-butoxycarbonyl)(methyl)amino)-3-vinyl-7,8-dihydro-4H-pyrazolo[1,5-a][1,4]diazepine-5(6H)-carboxylate: To a solution of tert-butyl 2-((tert-butoxycarbonyl)(methyl)amino)-3-iodo-7,8-dihydro-4H-pyrazolo[1,5-a][1,4]diazepine-5(6H)-carboxylate (550 mg, 1.0 equiv), potassium trifluoro(vinyl)borate (374 mg, 2.5 equiv), and Cs2CO₃ (1.09 g, 3.0 equiv) in dioxane (5.5 mL) and H2O (0.55 mL) was added Pd(dppf)C12 (40.9 mg, 0.05 equiv). The mixture was stirred at 90° C. for 2 hours. The mixture was diluted with water (10 mL) and extracted with ethyl acetate (3×10 mL). The combined organic layers were dried over anhydrous sodium sulfate, concentrated, and purified by reversed phase flash chromatography [C18, 0.1% formic acid condition] to afford the title compound (330 mg, 74% yield) as yellow solid; LCMS (ESI, M+1): m/z=393.2.

Step E. tert-butyl 2-((tert-butoxycarbonyl)(methyl)amino)-3-ethyl-7,8-dihydro-4H-pyrazolo[1,5-a][1,4]diazepine-5(6H)-carboxylate: To a solution of tert-butyl 2-((tert-butoxycarbonyl)(methyl)amino)-3-vinyl-7,8-dihydro-4H-pyrazolo[1,5-a][1,4]diazepine-5(6H)-carboxylate (330 mg, 1.0 equiv) in MeOH (3 mL) was added Pd/C (60 mg, 10% purity, 1.0 equiv) under N₂. The suspension was degassed under vacuum and purged with H₂ several times. The mixture was stirred under H₂ (15 psi) at 25° C. for 2 hours. The reaction was filtered, and the filtrate was concentrated to afford the title compound (300 mg, 90% yield) as yellow liquid; LCMS (ESI, M+1): m/z=395.1.

Step F. 3-ethyl-N-methyl-5,6,7,8-tetrahydro-4H-pyrazolor1,5-air1,41diazepin-2-amine: To a solution of tert-butyl 2-((tert-butoxycarbonyl)(methyl)amino)-3-ethyl-7,8-dihydro-4H-pyrazolo[1,5-a][1,4]diazepine-5(6H)-carboxylate (270 mg, 1.0 equiv) in MeCN (1 mL) was added HCl•dioxane (4 M, 2 mL, 12 equiv). The mixture was stirred at 0° C. for 0.5 hours. The mixture was concentrated. The residue was dissolved in MeOH and adjusted to pH >7 with NaHCO₃solid, then filtered and concentrated. The residue was dissolved in DCM/MeOH=10/1 and stirred for 10 minutes at 25° C. The mixture was then filtered and concentrated to afford the title compound (150 mg, crude) as yellow liquid.

The last two steps were performed according to Example 248. The title compound was obtained as yellow solid (FA salt). ¹H NMR (400 MHz, METHANOL-d₄) δ=7.50 (dd, J=5.6, 8.8 Hz, 1H), 7.14 (t, J=9.6 Hz, 1H), 6.96 (br d, J=3.2 Hz, 2H), 5.45-5.19 (m, 1H), 4.99-4.89 (m, 2H), 4.55 (dd, J=4.8, 16.0 Hz, 1H), 4.30-4.18 (m, 2H), 4.17-4.00 (m, 4H), 3.86-3.75 (m, 1H), 3.65 (br d, J=17.6 Hz, 1H), 3.52-3.33 (m, 5H), 3.25-3.04 (m, 3H), 2.79 (s, 3H), 2.73-2.61 (m, 1H), 2.43-2.34 (m, 2H), 2.34-2.14 (m, 3H), 2.14-1.97 (m, 4H), 1.96-1.83 (m, 1H), 1.11 (t, J=7.2 Hz, 3H), 1.03 (t, J=7.2 Hz, 3H); LCMS (ESI, M+1): m/z=673.4.

### Example 405

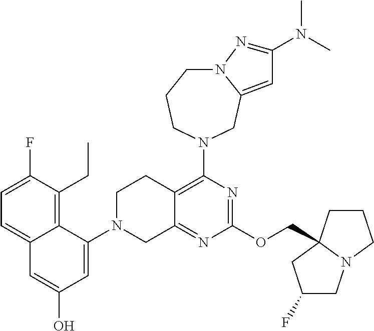 [SMILES: CCc1c(F)ccc2cc(O)cc(N3CCc4c(nc(OC[C@@]56CCCN5C[C@H](F)C6)nc4N4CCCn5nc(N(C)C)cc5C4)C3)c12]

### 4-(4-(2-(dimethylamino)-7,8-dihydro-4H-pyrazolo[1,5-a][1,4]diazepin-5(6H)-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl -6-fluoronaphthalen-2-ol

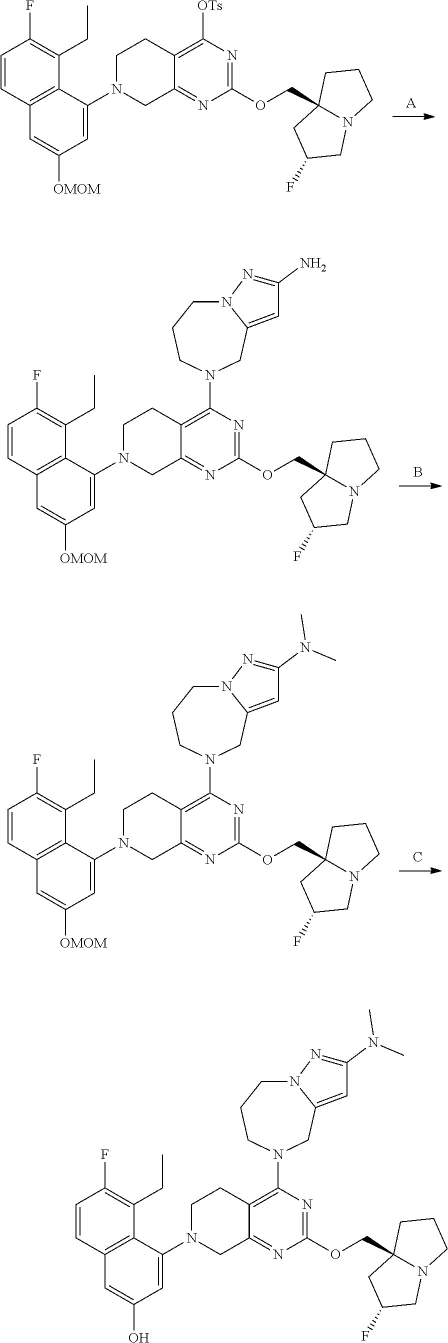

Step A. 5-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-amine: To a mixture of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (100 mg, 1.0 equiv) and 5,6,7,8-tetrahydro-4H-pyrazolo[1,5-a][1,4]diazepin-2-amine (26.3 mg, 1.2 equiv) in DMF (0.6 mL) was added N,N-diethylpropan-2-amine (37.2 mg, 2.0 equiv). The reaction was stirred at 40° C. for 12 hours. The mixture was filtered and purified by reversed phase flash chromatography to afford the title compound (90.0 mg, 83% yield) as white solid; LCMS (ESI, M+1): m/z=675.3.

Step B. 5-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d] pyrimidin-4-yl)-N,N-dimethyl-5,6,7,8-tetrahydro-4H-pyrazolo[1,5-a][1,4]diazepin-2-amine: A solution of (CHO)n (300 mg) in MeOH (1 mL) was stirred at 60° C. for 2 hours. A mixture of 5-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-amine (90.0 mg, 1 equiv), tetraisopropoxytitanium (113.7 mg, 3 equiv), and sodium cyanoborohydride (75.2 mg, 6 equiv) in MeOH (1 mL) was added to the mixture. The reaction was stirred at 25° C. for 12 hours. The solvent was removed and purified by column chromatography (Al₂O₃, Petroleum ether/Ethyl acetate=10/1 to 1/1) to afford the title compound (80.0 mg, 73% yield) as white solid; LCMS (ESI, M+1): m/z=703.2.

Step C. 4-(4-(2-(dimethylamino)-7,8-dihydro-4H-pyrazolo[1,5-a][1,4]diazepin-5(6H)-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-ol: To a solution of 5-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepin-2-amine (80.0 mg, 1.0 equiv) in MeOH (1 mL) was added HCl/MeOH (4 M, 1 mL). The reaction was stirred at 0° C. for 1 hour. The mixture was quenched with saturated NaHCO₃ solution (5.0 mL) and extracted with EtOAc (2×5.0 mL). The combined organic layers were dried, concentrated, and purified with HPLC to afford the title compound (9.00 mg, 11.7% yield) as off-white solid; ^{l}H NMR (400 MHz, METHONL-d) δ=7.74 (br d, J=1.6 Hz, 4H), 7.43 (br s, 6H), 3.80-3.72 (m, 1H), 3.65 (s, 3H), 3.58-3.55 (m, 1H), 3.04 (td, J=6.6, 10.6 Hz, 1H), 2.92-2.81 (m, 1H), 2.69 (td, J=5.6, 10.8 Hz, 1H), 2.45-2.35 (m, 1H), 2.24-2.13 (m, 1H), 2.07-1.98 (m, 1H), 1.86-1.72 (m, 4H), 1.62-1.56 (m, 1H), 1.07 (br s, 9H); LCMS (ESI, M+1): m/z=659.5.

### Example 406

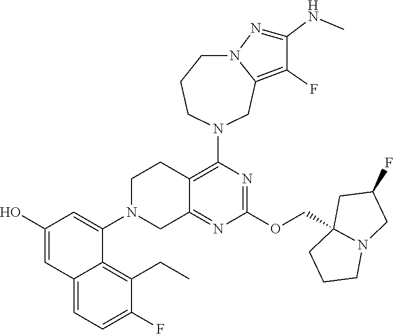 [SMILES: CCc1c(F)ccc2cc(O)cc(N3CCc4c(nc(OC[C@@]56CCCN5C[C@H](F)C6)nc4N4CCCn5nc(NC)c(F)c5C4)C3)c12]

### 5-ethyl-6-fluoro-4-(4-(3-fluoro-2-(methylamino)-7,8-dihydro-4H-pyrazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidin-7(8H)-yl)naphthalen-2-ol

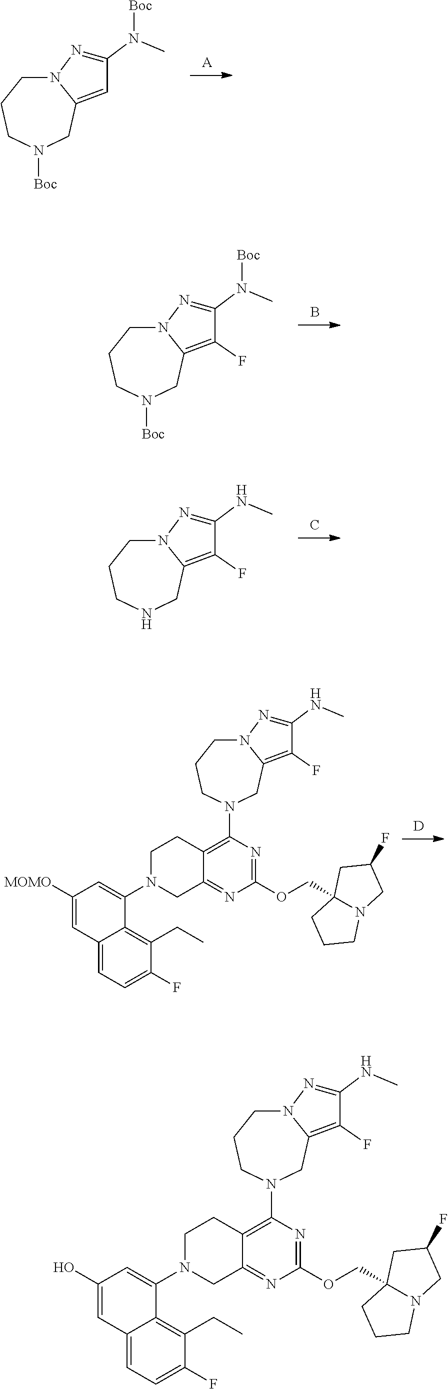

Step A. tert-butyl 2-((tert-butoxycarbonyl)(methyl)amino)-3-fluoro-7,8-dihydro-4H-pyrazolo[1,5-a][1,4]diazepine-5(6H)-carboxylate: To a solution of tert-butyl 2-((tert-butoxycarbonyl)(methyl)amino)-7,8-dihydro-4H-pyrazolo[1,5-a][1,4]diazepine-5(6H)-carboxylate (400 mg, 1.0 equiv) in ACN (4 mL) was added 1-(chloromethyl)-4-fluoro-1,4-diazoniabicyclo[2.2.2]octane;ditetrafluoroborate (1.93 g, 5.0 equiv). The reaction was stirred at 40° C. for 4 hours. The mixture was filtered, then the filtrate was concentrated and purified by prep-HPLC [column: Waters Xbridge C18 150×50 mm×10 μm; A: water (10 mM NH₄HCO₃), B: ACN, B %: 40%-70% over 10 minutes] to afford the title compound (95.0 mg, 23% yield) as yellow oil; LCMS (ESI, M+1): m/z=385.3.

Step B. 3-fluoro-N-methyl-5,6,7,8-tetrahydro-4H-pyrazolo[1,5-a][1,4]diazepin-2-amine: To a solution of tert-butyl 2-((tert-butoxycarbonyl)(methyl)amino)-3-fluoro-7,8-dihydro-4H-pyrazolo[1,5-a][1,4]diazepine-5(6H)-carboxylate (95.0 mg, 1.0 equiv) in MeOH (0.5 mL) was added HCl-MeOH (61.8 μL, 1.0 equiv) at 0° C. The mixture was stirred at 0° C. for 30 minutes. The mixture was concentrated to dryness. To the residue were added MeOH (5 mL) and NaHCO₃ (50.0 mg). The mixture was stirred and filtered. The filtrate was concentrated to afford the title compound (40.0 mg, crude) as yellow oil.

The last two steps were performed according to Example 248. The title compound was obtained as white solid (FA salt). ¹H NMR (400 MHz, METHANOL-d₄) δ=7.51 (dd, J=5.6, 9.2 Hz, 1H), 7.14 (t, J=9.2 Hz, 1H), 7.02-6.88 (m, 2H), 5.45-5.18 (m, 1H), 4.85-4.71 (m, 2H), 4.26-4.11 (m, 4H), 4.04 (br s, 3H), 3.66 (br d, J=17.6 Hz, 1H), 3.56-3.47 (m, 1H), 3.38 (br d, J=4.4 Hz, 4H), 3.28 (br d, J=8.0 Hz, 2H), 3.21-3.12 (m, 1H), 3.12-3.02 (m, 1H), 2.79 (s, 3H), 2.75 (br s, 1H), 2.42-2.09 (m, 4H), 2.09-1.84 (m, 4H), 1.10 (t, J=7.2 Hz, 3H); LCMS (ESI, M+1): m/z =663.4

### Example 407

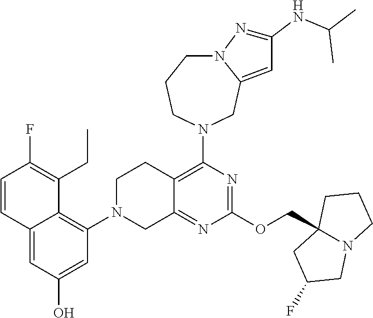 [SMILES: CCc1c(F)ccc2cc(O)cc(N3CCc4c(nc(OC[C@@]56CCCN5C[C@H](F)C6)nc4N4CCCn5nc(NC(C)C)cc5C4)C3)c12]

### 5-ethyl-6-fluoro-4-(2-(((2R,7aS)-2-fluorohexahydro-1H-pyrrolizin-7a-yl)methoxy)-4-(2-(isopropylamino)-7,8-dihydro-4H-pyrazolo[1,5-a][1,4]diazepin-5(6H)-yl)-5,6-dihydropyrido[3,4-d]pyrimidin-7(8H)-yl)naphthalen-2-ol

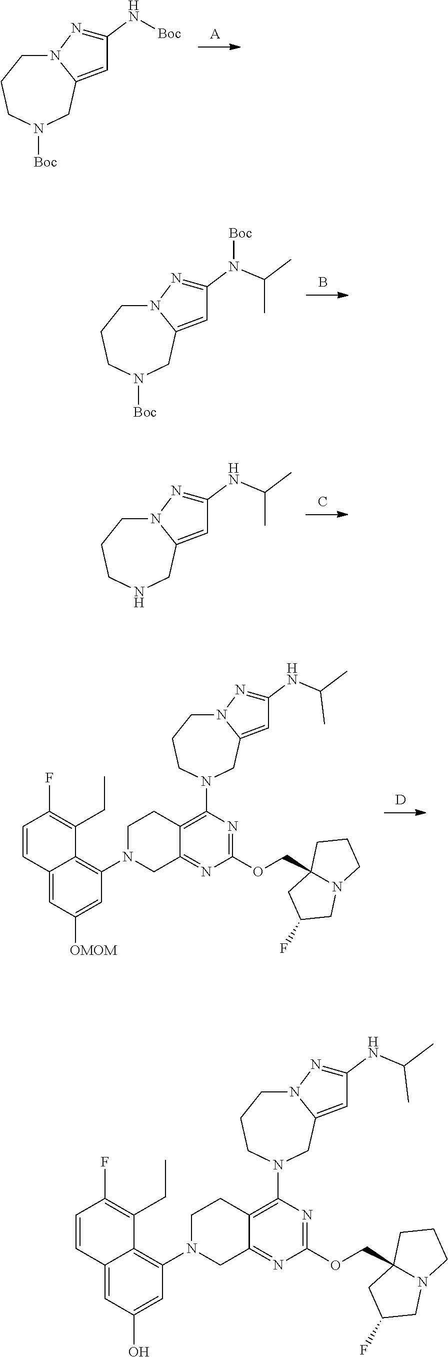

Step A. tert-butyl 2-((tert-butoxycarbonyl)(isopropyl)amino,)-7,8-dihydro-4H-pyrazolo[1,5-a][1,4]diazepine-5(6H)-carboxylate: To a mixture of tert-butyl 2-((tert-butoxycarbonyl)amino)-7,8-dihydro-4H-pyrazolo[1,5-a][1,4]diazepine-5(6H)-carboxylate (100 mg, 1.0 equiv) in THE (1 mL) was added NaH (56.7 mg, 60% purity, 5.0 equiv). The mixture was stirred at 25° C. for 1 hour. Then to the mixture was added 2-iodopropane (482 mg, 10 equiv) and the reaction was stirred at 60° C. for 12 hours under N₂ atmosphere. The mixture was stirred at 25° C. for 0.5 hours. The mixture was quenched with H₂O (3 mL) and extracted with EtOAc (3×5 mL). The combined organic layers were dried over anhydrous sodium sulfate, concentrated, and purified by reversed phase flash chromatography [C18, 0.1% formic acid condition] to afford the title compound (50.0 mg, 40% yield) as yellow oil; LCMS (ESI, M+1): m/z=395.2.

Step B. N-isopropyl-5,6,7,8-tetrahydro-4H-pyrazolo[1,5-a][1,4]diazepin-2-amine: To a solution of tert-butyl 2-((tert-butoxycarbonyl)(isopropyl)amino)-7,8-dihydro-4H-pyrazolo[1,5-a][1,4]diazepine-5(6H)-carboxylate (50.0 mg, 1.0 equiv) in MeCN (0.5 mL) was added HCl•dioxane (4.0 M, 2.0 mL). The mixture was stirred at 0° C. for 1 hour. The pH of the mixture was adjusted to 8 with saturated NaHCO₃ aqueous solution (3.0 mL) and the mixture was extracted with ethyl acetate (3×5 mL). The combined organic layers were dried over anhydrous sodium sulfate and concentrated to afford the title compound (23.0 mg, 89% yield) as yellow oil; LCMS (ESI, M+1): m/z=195.2.

The last two steps were performed according to Example 248. The title compound was obtained as yellow solid; ¹H NMR (400 MHz, METHANOL-d₄) δ=7.56-7.48 (m, 1H), 7.19-7.11 (m, 1H), 7.00-6.93 (m, 2H), 5.56 (d, J=2.8 Hz, 1H), 5.37-5.17 (m, 1H), 4.73-4.64 (m, 1H), 4.30-4.22 (m, 2H), 4.18-4.01 (m, 4H), 3.97-3.87 (m, 1H), 3.73-3.64 (m, 1H), 3.56-3.34 (m, 5H), 3.26-3.14 (m, 5H), 3.04-2.95 (m, 1H), 2.80-2.64 (m, 1H), 2.36-1.78 (m, 9H), 1.18-1.10 (m, 9H); LCMS (ESI, M+1): m/z=673.4.

### Example 408

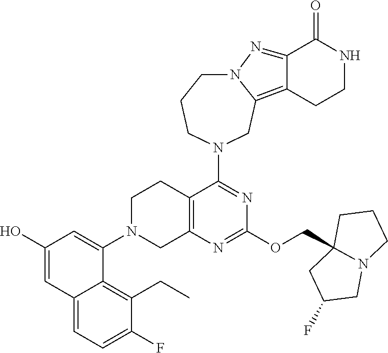 [SMILES: CCc1c(F)ccc2cc(O)cc(N3CCc4c(nc(OC[C@@]56CCCN5C[C@H](F)C6)nc4N4CCCn5nc6c(c5C4)CCNC6=O)C3)c12]

### 2-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-2,3,4,5,10,11-hexahydro-1H-pyrido[3′,4′:3,4]pyrazolo[1,5-a][1,4]diazepin-8(9H)-one

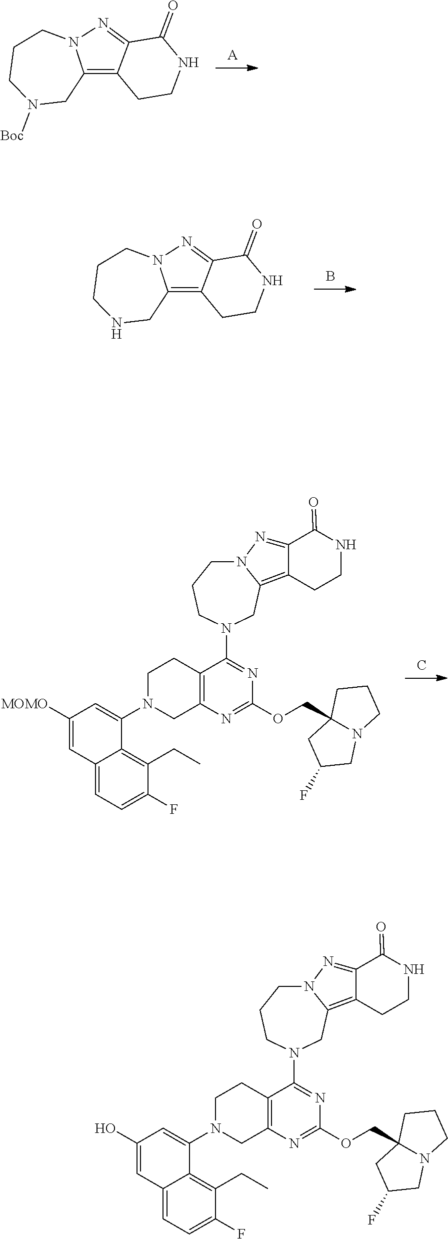

Step A. 2,3,4,5,10,11-hexahydro-1H-pyrido[3′,4′:3,4]pyrazolo[1,5-a][1,4]diazepin-8(9H)-one: To a solution of tert-butyl 6-oxo-5,8,9,13-tetrazatricyclo[7.5.0.02,7]tetradeca-1,7-diene-13-carboxylate (10.0 mg, 1.0 equiv) in MeCN (0.2 mL) was added HCl•dioxane (4 M, 0.4 mL). The mixture was stirred at 0° C. for 1 hour. The reaction was concentrated to afford the title compound (11 mg, crude) as yellow oil.

The last two steps were performed according to Example 248. The title compound was obtained as yellow solid; ¹H NMR (400 MHz, METHANOL-d₄) δ=7.54-7.47 (m, 1H), 7.18-7.11 (m, 1H), 6.98-6.93 (m, 2H), 5.34-5.30 (m, 1H), 5.33-5.30 (m, 1H), 5.18 (br s, 1H), 4.92 (br s, 1H), 4.77 (br d, J=3.4 Hz, 1H), 4.75-4.72 (m, 1H), 4.61-4.47 (m, 3H), 4.15 (br s, 1H), 4.08-3.97 (m, 4H), 3.75-3.62 (m, 2H), 3.53-3.48 (m, 2H), 3.45-3.40 (m, 2H), 3.18 (br d, J=7.6 Hz, 3H), 3.10 (br s, 2H), 3.01-2.95 (m, 1H), 2.94-2.87 (m, 1H), 2.79-2.65 (m, 2H), 2.33-2.18 (m, 2H), 2.15-2.07 (m, 2H), 2.04 (br dd, J=2.4, 11.1 Hz, 1H), 1.99-1.90 (m, 2H), 1.87-1.75 (m, 1H), 1.15-1.04 (m, 3H); LCMS (ESI, M+1): m/z=685.5.

### Example 409

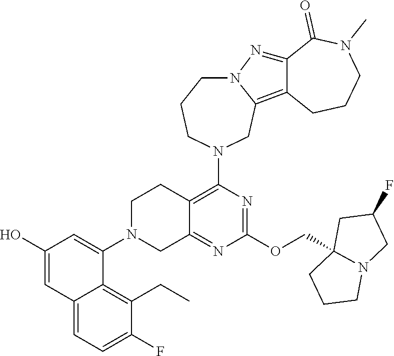 [SMILES: CCc1c(F)ccc2cc(O)cc(N3CCc4c(nc(OC[C@@]56CCCN5C[C@H](F)C6)nc4N4CCCn5nc6c(c5C4)CCCN(C)C6=O)C3)c12]

### 2-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-9-methyl-2,3,4,5,9,10,11,12-octahydroazepino[3′,4′:3,4]pyrazolo[1,5-a][1,4]diazepin-8(1H)-one

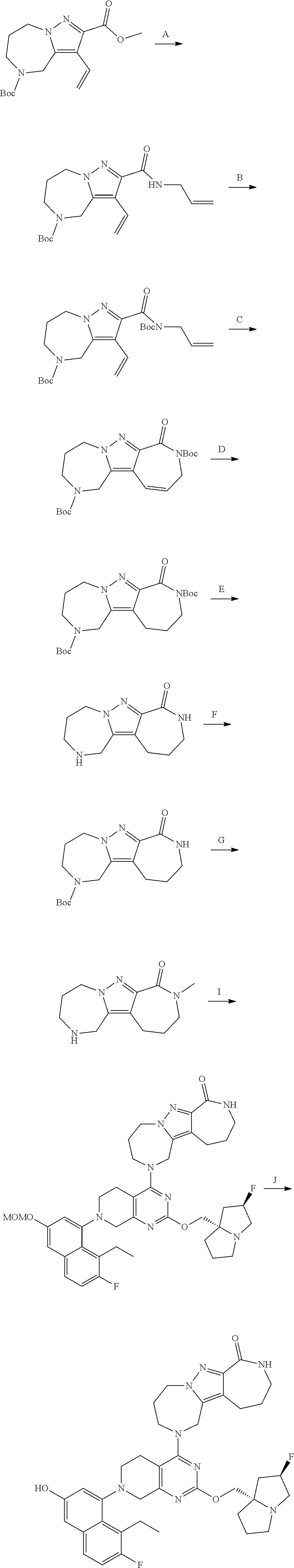

Step A. tert-butyl 2-(allylcarbamoyl)-3-vinyl-7,8-dihydro-4H-pyrazolo[1,5-a][1,4]diazepine-5(6H)-carboxylate: A mixture of 05-tert-butyl 02-methyl 3-vinyl-4,6,7,8-tetrahydropyrazolo[1,5-a][1,4]diazepine-2,5-dicarboxylate (900 mg, 1.0 equiv), prop-2-en-1-amine (2.62 g, 10 equiv, HCl), and TEA (2.83 g, 10 equiv) was stirred at 100° C. for 6 hours. The reaction was diluted with EtOAc (30 mL) and water (40 mL) and extracted with EtOAc (20 mL). The combined organic layers were washed with brine (20 mL), dried over anhydrous sodium sulfate, concentrated, and purified by reversed phase flash chromatography [water (0.1% F A)/acetonitrile] to afford the title compound (560 mg, 57% yield) as yellow solid; ¹H NMR (400 MHz, CHLOROFORM-d) δ=7.19-7.10 (m, 1H), 7.13 (dd, J=11.6, 18 Hz, 1H), 6.96 (br d, J=8.0 Hz, 1H), 5.99-5.86 (m, 1H), 5.46-5.33 (m, 2H), 5.26 (dd, J=1.2, 17.2 Hz, 1H), 5.16 (dd, J=0.8, 10.2 Hz, 1H), 4.58 (br s, 2H), 4.43-4.33 (m, 2H), 4.03 (br t, J=5.6 Hz, 2H), 3.74 (br s, 2H), 1.98 (br d, J=4.4 Hz, 2H), 1.38 (br s, 9H); LCMS (ESI, M+1): m/z=347.3.

Step B. tert-butyl 2-(allyl(tert-butoxycarbonyl)carbamoyl)-3-vinyl-7,8-dihydro-4H-pyrazolo[1,5-a][1,4]diazepine-5(6H)-carboxylate: To a solution of tert-butyl 2-(allylcarbamoyl)-3-vinyl-4,6,7,8-tetrahydropyrazolo[1,5-a][1,4]diazepine-5-carboxylate (490 mg, 1.0 equiv) in MeCN (10 mL) were added DMAP (173 mg, 1.0 equiv) and (Boc)20 (617 mg, 2.0 equiv). The reaction was stirred at 40° C. for 16 hours. The mixture was concentrated and purified by column chromatography (SiO2, petroleum ether/ethyl acetate 10/1 to 1/1) to afford the title compound (550 mg, 68.6% yield) as green oil; LCMS (ESI, M-99): m/z=347.3.

Step C. di-tert-butyl 8-oxo-4,5,8,10-tetrahydroazepino[3′,4′:3,4]pyrazolo[1,5-a][1,4]diazepine-2,9(1H,3H)-dicarboxylate: A mixture of tert-butyl 2-[allyl(tert-butoxycarbonyl)carbamoyl]-3-vinyl-4,6,7,8-tetrahydropyrazolo[1,5-a][1,4]diazepine-5-carboxylate (550 mg, 1.0 equiv) and benzylidene-[1,3-bis(2,4,6-trimethylphenyl)imidazolidin-2-ylidene]-dichloro-ruthenium;tricyclohexylphosphane (105 mg, 0.1 equiv) in DCM (20 mL) was degassed and purged with N₂ 3 times. The mixture was stirred at 40° C. for 16 hours under N₂ atmosphere. The reaction was diluted with EtOAc (5 mL) and water (10 mL) and extracted with EtOAc (5 mL). The combined organic layers were washed with brine (5 mL), dried over anhydrous sodium sulfate, concentrated, and purified by column chromatography (SiO₂, Petroleum ether: Ethyl acetate=5:1 to 1:1) to afford the title compound (340 mg, 65.7% yield) as yellow solid; i H NMR (400 MHz, METHANOL-d₄) δ=6.99-6.88 (m, 1H), 6.32-6.22 (m, 1H), 4.64-4.54 (m, 4H), 4.20-4.13 (m, 2H), 3.79 (br s, 2H), 1.96-1.88 (m, 2H), 1.53 (s, 9H), 1.41 (s, 9H); LCMS (ESI, M+1): m/z=319.2.

Step D. di-tert-butyl 8-oxo-4,5,8,10,11,12-hexahydroazepino[3′,4′:3,4]pyrazolo[1,5-a][1,4]diazepine-2,9(1H,3H)-dicarboxylate: To a solution of ditert-butyl 11-oxo-4,8,9,12-tetrazatricyclo[8.5.0.02,8]pentadeca-1,9,14-triene-4,12-dicarboxylate (330 mg, 1.0 equiv) in MeOH (10 mL) was added Pd/C (50 mg, 10% purity) under N₂ atmosphere. The suspension was degassed and purged with H₂ 3 times. The mixture was stirred under H₂ (15 Psi) at 20° C. for 3 hours. The mixture was filtered, and the filtrate was concentrated to afford the title compound (330 mg, crude) as white solid; LCMS (ESI, M+1): m/z=321.2.

Step E. 2,3,4,5,9,10,11,12-octahydroazepino[3′,4′:3,4]pyrazolo[1,5-a][1,4]diazepin-8(1H)-one: To a solution of ditert-butyl 11-oxo-4,8,9,12-tetrazatricyclo[8.5.0.02,8]pentadeca-1,9-diene-4,12-dicarboxylate (80 mg, 1.0 equiv) in MeCN (0.5 mL) was added HCl•dioxane (4 M, 1 mL). The reaction was stirred at 0° C. for 1 hour. The mixture was concentrated under vacuum to afford the title compound (41 mg, crude) as white solid.

Step F. tert-butyl 8-oxo-3,4,5,8,9,10,11,12-octahydroazepino[3′,4′:3,4]pyrazolo[1,5-a][1,4]diazepine-2(1H)-carboxylate: To a solution of 4,8,9,12-tetrazatricyclo[8.5.0.02,8]pentadeca-1,9-dien-11-one (30 mg, 1.0 equiv) in DCM (2 mL) were added (Boc)20 (44.6 mg, 1.5 equiv) and TEA (41.3 mg, 3.0 equiv). The reaction was stirred at 20° C. for 2 hours. The mixture was concentrated and purified by reversed phase flash chromatography [water (0.1% FA)/acetonitrile] to afford the title compound (43 mg, 98.2% yield) as white solid; ¹H NMR (400 MHz, METHANOL-d₄) δ=4.53-4.44 (m, 4H), 3.76 (br s, 2H), 3.32 (br s, 2H), 2,88 (m, 2H), 2.08-2.00 (m, 2H), 1.88 (br s, 2H), 1.43 (br d, J=10.4 Hz, 9H); LCMS (ESI, M+1): m/z=321.2.

Step G. tert-butyl 9-methyl-8-oxo-3,4,5,8,9,10,11,12-octahydroazepino[3′,4′:3,4]pyrazolo[1,5-a][1,4]diazepine-2(1H)-carboxylate: To a solution of tert-butyl 11-oxo-4,8,9,12-tetrazatricyclo[8.5.0.02,8]pentadeca-1,9-diene-4-carboxylate (47 mg, 1.0 equiv) in TUE (1 mL) was added NaH (11.7 mg, 60% purity, 2.0 equiv). The mixture was stirred at 0° C. for 0.5 hours. Then MeI (62.5 mg, 3.0 equiv) was added into the mixture and the mixture was stirred at 20° C. for 16 hours. The mixture was quenched with water (10 mL) and extracted with dichloromethane (2×10 mL). The combined organic layers were dried over anhydrous sodium sulfate and concentrated to afford the title compound (50 mg, crude) as white solid; ¹H NMR (400 MHz, CHLOROFORM-d) δ=4.52-4.47 (m, 1H), 4.52-4.46 (m, 1H), 4.39 (s, 2H), 3.79-3.67 (m, 2H), 3.38 (br s, 2H), 3.16 (s, 3H), 2.87-2.67 (m, 2H), 2.12-2.01 (m, 2H), 2.00-1.83 (m, 2H), 1.42 (br d, J=6.4 Hz, 9H); LCMS (ESI, M+1): m/z=335.2.

Step H. 9-methyl-2,3,4,5,9,10,11,12-octahydroazepino[3′,4′:3,4]pyrazolo[1,5-a][l,4]diazepin-8(1H)-one: To a solution of tert-butyl 12-methyl-11-oxo-4,8,9,12-tetrazatricyclo[8.5.0.02,8]pentadeca-1,9-diene-4-carboxylate (60 mg, 1.0 equiv) in MeCN (0.5 mL) was added HCl•dioxane (4 M, 1 mL). The reaction was stirred at 0° C. for 1 hour. The mixture was concentrated under vacuum to afford the title compound (49 mg, crude, HCl) as yellow oil.

The last two steps were performed according to Example 248. The title compound was obtained as white solid; ¹H NMR (400 MHz, METHANOL-d₄) δ=7.51 (dd, J=6.0, 9.2 Hz, 1H), 7.14 (t, J=9.2 Hz, 1H), 6.95 (s, 2H), 5.36-5.15 (m, 1H), 4.96 (dd, J=6.4, 16.4 Hz, 1H), 4.69 (dd, J=4.4, 16.0 Hz, 1H), 4.61-4.43 (m, 2H), 4.15-3.91 (m, 5H), 3.69 (d, J=17.6 Hz, 1H), 3.53-3.37 (m, 5H), 3.21-3.10 (m, 8H), 3.01-2.81 (m, 2H), 2.78-2.61 (m, 2H), 2.31-2.22 (m, 1H), 2.21-2.13 (m, 2H), 2.13-2.07 (m, 2H), 2.06-1.97 (m, 2H), 1.96-1.86 (m, 2H), 1.85-1.72 (m, 1H), 1.10 (t, J=7.2 Hz, 3H); LCMS (ESI, M+1): m/z=713.5.

### Example 410

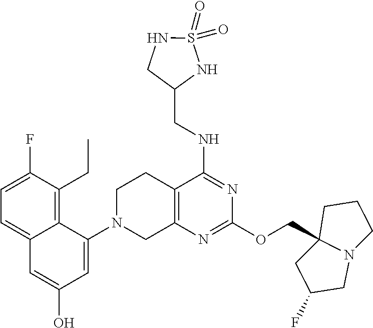 [SMILES: CCc1c(F)ccc2cc(O)cc(N3CCc4c(nc(OC[C@@]56CCCN5C[C@H](F)C6)nc4NCC4CNS(=O)(=O)N4)C3)c12]

### 3-(((7-(8-ethyl-7-fluoro-3-hydroxynaphthalen-1-yl)-2-(((2R,7aS)-2-fluorohexahydro-1H-pyrrolizin-7a-yl)methoxy)-5, 6,7,8-tetrahydropyrido[3,4-d]pyrimidin-4-yl)amino)methyl)-1,2,5-thiadiazolidine 1,1-dioxide

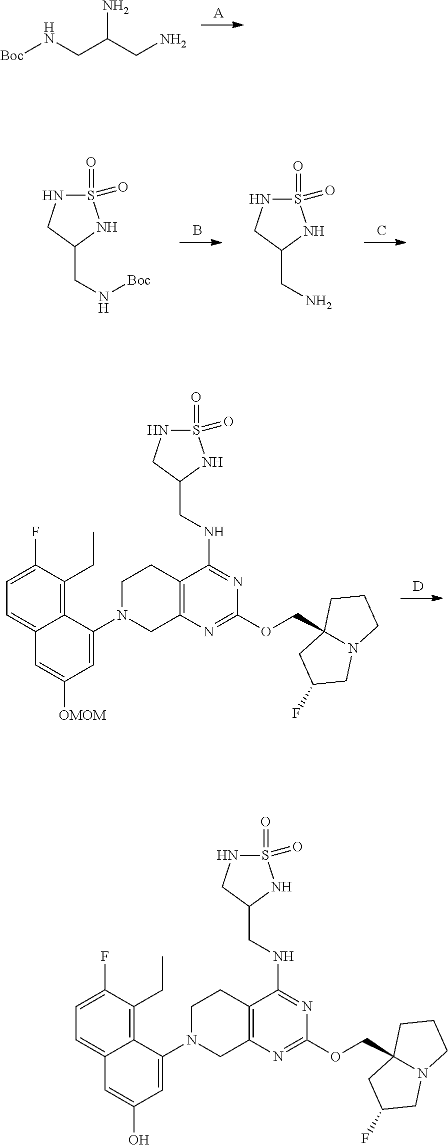

Step A. tert-butyl ((1,1-dioxido-1,2,5-thiadiazolidin-3-yl)methyll)carbamate: To a mixture of tert-butyl (2,3-diaminopropyl)carbamate (400 mg, 1.0 equiv) in pyridine (4 mL) was added dropwise sulfamide (203 mg, 1.0 equiv) at 25° C. The reaction was stirred at 115° C. for 20 hours. The mixture was filtered, concentrated and purified by prep-TLC (dichloromethane/methanol 10/1) to afford the title compound (100 mg, 16% yield over two steps) as yellow oil; ¹H NMR (400 MHz, dimethylsulfoxide-d₆) δ=6.96-6.85 (m, 3H), 3.65-3.55 (m, 1H), 3.33-3.30 (m, 1H), 3.09-2.97 (m, 3H), 1.38 (s, 9H).

Step B. 3-(aminomethyl)-1,2,5-thiadiazolidine 1,1-dioxide: To a mixture of tert-butyl ((1,1-dioxido-1,2,5-thiadiazolidin-3-yl)methyl)carbamate (41.0 mg, 1.0 equiv) in DCM (0.50 mL) was added HCl•dioxane (4 M, 12.6 equiv) at 0° C. The reaction was stirred at 25° C. for 1 hour. The mixture was concentrated to afford the title compound (30.0 mg, 98% yield, HCl) as brown solid.

The last two steps were performed according to Example 248. The title compound was obtained as orange solid (FA salt). ¹H NMR (400 MHz, dimethylsulfoxide-d₆) δ=9.98-9.47 (m, 1H), 7.61-7.55 (m, 1H), 7.26-7.19 (m, 1H), 7.15-7.08 (m, 1H), 7.03-6.89 (m, 4H), 5.34-5.15 (m, 1H), 3.97-3.88 (m, 2H), 3.86-3.79 (m, 1H), 3.78-3.69 (m, 1H), 3.66-3.53 (m, 2H), 3.52-3.38 (m, 4H), 3.24-3.09 (m, 4H), 3.09-3.02 (m, 2H), 3.00-2.96 (m, 1H), 2.84-2.75 (m, 1H), 2.70-2.58 (m, 1H), 2.09-1.94 (m, 3H), 1.85-1.78 (m, 1H), 1.76-1.66 (m, 2H), 1.03-0.94 (m, 3H); ¹⁹F NMR (400 MHz, dimethylsulfoxide-d₆) δ=−121.413, -171.975; LCMS (ESI, M+1): m/z=630.3.

### Example 411

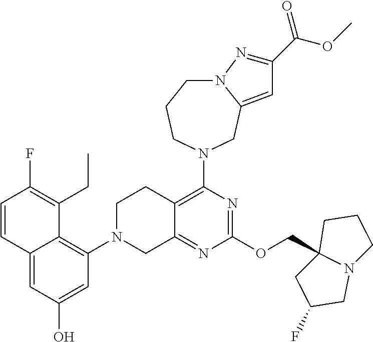 [SMILES: CCc1c(F)ccc2cc(O)cc(N3CCc4c(nc(OC[C@@]56CCCN5C[C@H](F)C6)nc4N4CCCn5nc(C(=O)OC)cc5C4)C3)c12]

### methyl 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepine-2-carboxylate

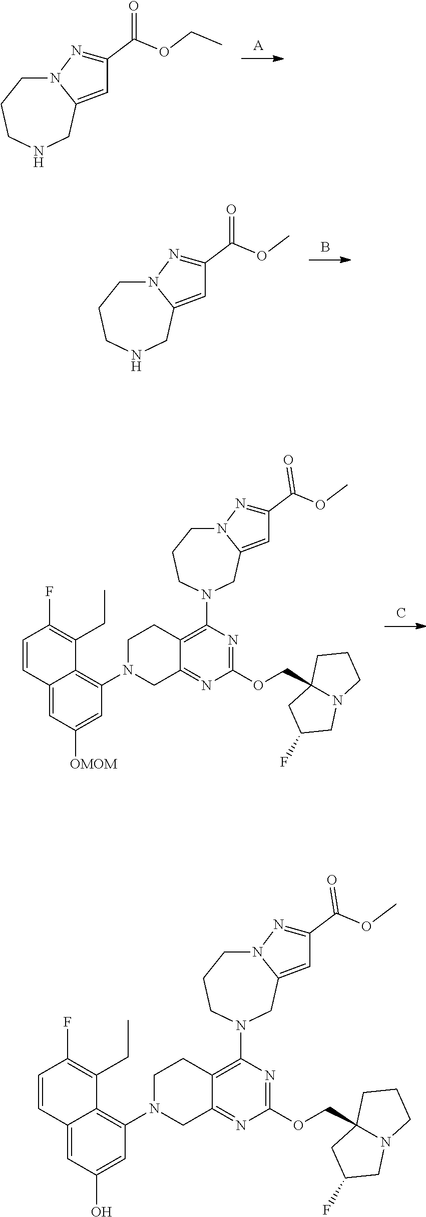

Step A. methyl 5,6,7,8-tetrahydro-4H-pyrazolo[1,5-a][1,4]diazepine-2-carboxylate: To a mixture of ethyl 5,6,7,8-tetrahydro-4H-pyrazolo[1,5-a][1,4]diazepine-2-carboxylate (20.0 mg, 1.0 equiv) in MeOH (1 mL) was added NaOMe (15.5 mg, 3.0 equiv). The reaction was stirred at 25° C. for 2 hours. The pH of the mixture was adjusted to 7 by dropwise HCl (1 M). The mixture was filtered and purified by prep-HPLC (column: Welch Xtimate C18 150*25 mm*5 um; mobile phase: [water (NH₃H₂O)-ACN]; B %: 0%-28%, 8 min) to afford the title compound (100 mg, 54% yield) as white oil. LCMS (ESI, M+1): m/z=196.1.

The last two steps were performed according to Example 248. The title compound was obtained as yellow solid (FA salt). ¹H NMR (400 MHz, METHANOL-d₄) δ=7.52 (dd, J=5.6, 8.8 Hz, 1H), 7.15 (t, J=9.2 Hz, 1H), 6.98 (br d, J=4.8 Hz, 2H), 6.80 (d, J=2.4 Hz, 1H), 5.45-5.27 (m, 1H), 5.07-4.99 (m, 1H), 4.81 (br s, 1H), 4.57 (br d, J=4.2 Hz, 2H), 4.30-4.09 (m, 3H), 4.36-3.94 (m, 2H), 3.88 (s, 3H), 3.69-3.52 (m, 2H), 3.49-3.35 (m, 6H), 3.25-3.11 (m, 2H), 2.82-2.68 (m, 1H), 2.46-2.25 (m, 3H), 2.24-2.15 (m, 1H), 2.13-1.83 (m, 4H), 1.11 (br t, J=6.4 Hz, 3H). LCMS (ESI, M+1): m/z=674.4.

### Example 412

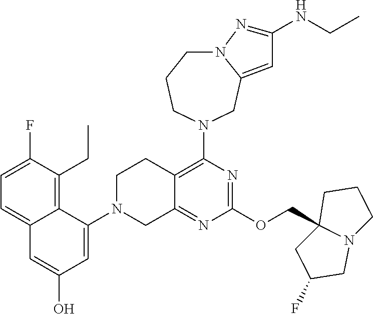 [SMILES: CCNc1cc2n(n1)CCCN(c1nc(OC[C@@]34CCCN3C[C@H](F)C4)nc3c1CCN(c1cc(O)cc4ccc(F)c(CC)c14)C3)C2]

### 5-ethyl-4-(4-(2-(ethylamino)-7,8-dihydro-4H-pyrazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidin-7(8H)-yl)-6-fluoronaphthalen-2-ol

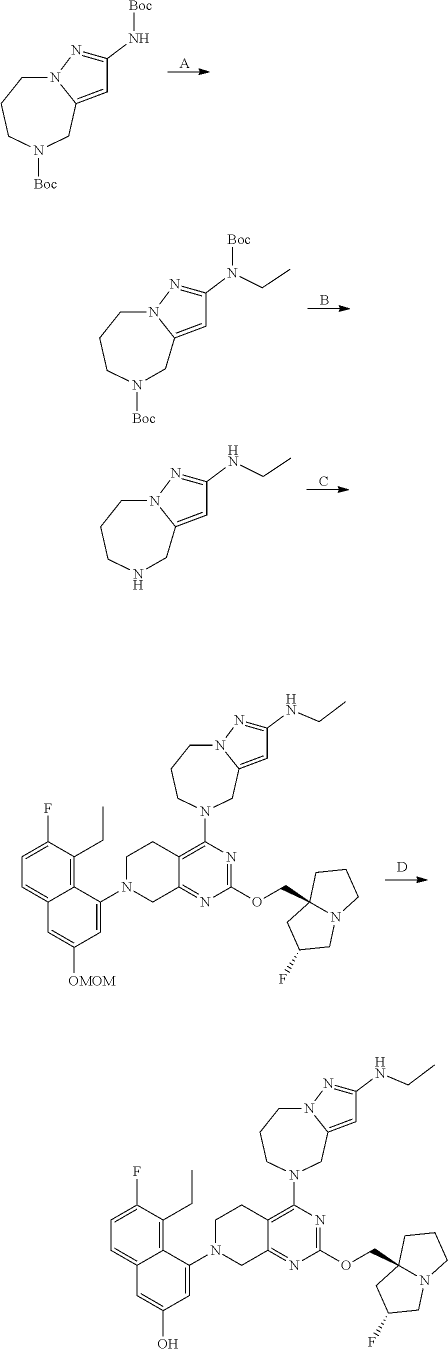

Step A. tert-butyl 2-((tert-butoxycarbonyl)(ethyl)amino)-7,8-dihydro-4H-pyrazolo[1,5-al[1,4]diazepine-5(6H)-carboxylate: To a solution of tert-butyl 2-(tert-butoxycarbonylamino)-4,6,7,8-tetrahydropyrazolo[1,5-a][1,4]diazepine-5-carboxylate (200 mg, 1.0 equiv) in DMF (1.5 mL) were added EtI (265 mg, 3.0 equiv) and Cs₂CO₃ (555 mg, 3.0 equiv). The mixture was stirred at 20° C. for 10 hours. The mixture was diluted with water (30 mL) and extracted with ethyl acetate (3×20 mL). The organic phase was dried over Na₂SO₄, concentrated, and purified with reversed phase flash chromatography {C18, 0.1% formic acid] to afford the title compound (200 mg, 92% yield) as yellow solid; LCMS (ESI, M+1): m/z=381.3.

Step B. N-ethyl-5,6,7,8-tetrahydro-4H-pyrazolo[1,5-a][1,4]diazepin-2-amine: To a solution of tert-butyl 2-[tert-butoxycarbonyl(ethyl)amino]-4,6,7,8-tetrahydropyrazolo[1,5-a][1,4]diazepine-5-carboxylate (190 mg, 1.0 equiv) in MeOH (0.9 mL) was added HCl•dioxane (4 M, 2 mL, 16 equiv) at 0° C. The mixture was stirred at 20° C. for 2 hours. To the mixture was added NaHCO₃The mixture was diluted with water (30 mL) and extracted with ethyl acetate (4×30 mL) at 0° C. The organic phase was dried over Na₂SO₄ and concentrated to afford the title compound (100 mg, 98% yield) as yellow oil; LCMS (ESI, M+1): m/z=181.2.

The last two steps were performed according to Example 248. The title compound was obtained as yellow solid (FA salt). 1H NMR (400 MHz, METHANOL-d₄) δ=8.50 (s, 1H), 7.52 (dd, J=6.0, 9.0 Hz, 1H), 7.15 (t, J=9.2 Hz, 1H), 6.97 (s, 2H), 5.56 (s, 1H), 5.50-5.29 (m, 1H), 4.83 (br d, J=8.0 Hz, 1H), 4.69 (dd, J=6.0, 16.4 Hz, 1H), 4.35-4.28 (m, 1H), 4.27-4.17 (m, 3H), 4.17-4.01 (m, 2H), 3.99-3.86 (m, 1H), 3.70 (br d, J=17.6 Hz, 1H), 3.64-3.45 (m, 4H), 3.45-3.35 (m, 2H), 3.27-3.15 (m, 3H), 3.10 (q, J=7.2 Hz, 2H), 2.81-2.64 (m, 1H), 2.55-1.91 (m, 8H), 1.18 (t, J=7.2 Hz, 3H), 1.12 (t, J=6.8 Hz, 3H); LCMS (ESI, M+1): m/z=659.4.

### Example 413

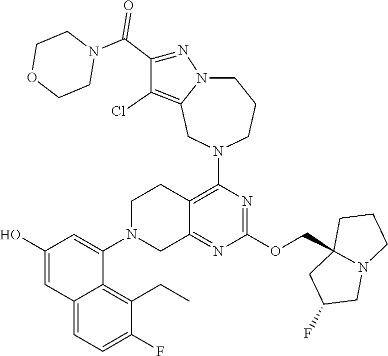 [SMILES: CCc1c(F)ccc2cc(O)cc(N3CCc4c(nc(OC[C@@]56CCCN5C[C@H](F)C6)nc4N4CCCn5nc(C(=O)N6CCOCC6)c(Cl)c5C4)C3)c12]

### (3-chloro-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(morpholino)methanone

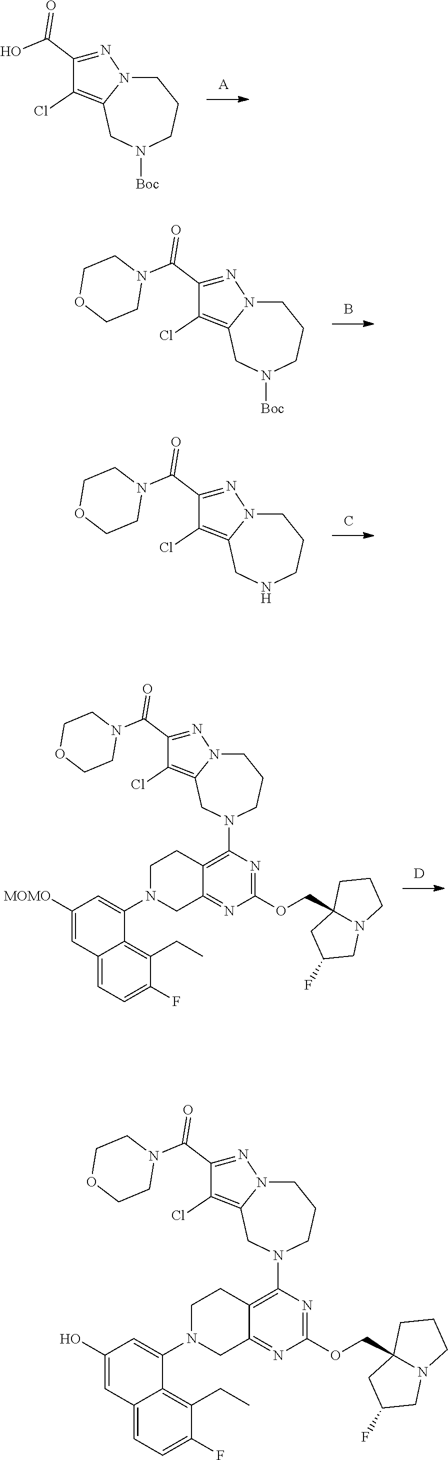

Step A. tert-butyl 3-chloro-2-(morpholine-4-carbonyl)-7,8-dihydro-4H-pyrazolo[1,5-a][1,4]diazepine-5(6H)-carboxylate: To a solution of 5-(tert-butoxycarbonyl)-3-chloro-5,6,7,8-tetrahydro-4H-pyrazolo[1,5-a][1,4]diazepine-2-carboxylic acid (32.5 g, 1.0 equiv), morpholine (11.7 g, 1.3 equiv), and N,N-diethylpropan-2-amine (33.3 g, 2.5 equiv) in DMF (130 mL) was added HATU (78.3 g, 2.0 equiv). The mixture was stirred at 15° C. for 2 hours. The mixture was diluted with water (1000 mL) and extracted with ethyl acetate (2×300 mL). The organic layer was washed with brine (300 mL) and dried over Na₂SO₄. The organic layer was concentrated and purified by column chromatography [SiO₂, Petroleum ether/Ethyl acetate=10/1 to 1/3] and reversed phase flash chromatography [C18, 0.1% formic acid condition] to afford the title compound (20.2 g, 51% yield) as white solid; ¹H NMR (400 MHz, METHANOL-d₄) δ=4.59 (s, 2H), 4.53-4.41 (m, 2H), 3.74 (m, 6H), 3.69-3.57 (m, 4H), 1.97-1.83 (m, 2H), 1.44 (s, 9H); LCMS (ESI, M+1): m/z=385.2.

Step B. (3-chloro-5,6,7,8-tetrahydro-4H-pyrazolo[1,5-a][1,4]diazepin-2-yl)(morpholino)methanone: To a solution of tert-butyl 3-chloro-2-(morpholine-4-carbonyl)-7,8-dihydro-4H-pyrazolo[1,5-a][1,4]diazepine-5(6H)-carboxylate (12.0 g, 1.0 equiv) in ACN (60 mL) was added HCl•dioxane (4 M, 62 mL, 8.0 equiv) at 0° C. The mixture was stirred at 0° C. for 0.5 hours. The mixture was concentrated to give a residue. The residue was adjusted to pH to 9 with saturated Na₂CO₃ and then extracted with DCM (6×60 mL). The combine organic layers were dried over Na₂SO₄ and concentrated to afford the title compound (8.90 g, 99% yield) as white solid; ¹H NMR (400 MHz, CHLOROFORM-d) δ=4.42-4.32 (m, 2H), 3.91 (m, 2H), 3.75 (br s, 4H), 3.70-3.59 (m, 4H), 3.27-3.13 (m, 2H), 1.92-1.81 (m, 2H).

The last two steps were performed according to Example 248. The title compound was obtained as yellow solid; ^{l}H NMR (400 MHz, METHANOL-d₄) δ=9.15-9.05 (m, 1H), 7.68 (dd, J=6.0, 9.0 Hz, 1H), 7.35-7.18 (m, 2H), 7.07 (d, J=1.8 Hz, 1H), 4.72-4.56 (m, 1H), 4.52-4.20 (m, 3H), 4.11 (br d, J=5.2 Hz, 1H), 4.04-3.91 (m, 1H), 3.89-3.64 (m, 2H), 3.13 (br s, 2H), 2.91 (br d, J=12.8 Hz, 7H), 2.55-2.34 (m, 1H), 2.30-2.02 (m, 6H), 1.99-1.68 (m, 7H), 0.80 (q, J =7.6 Hz, 3H); LCMS (ESI, M+1): m/z=763.4.

### Example 414

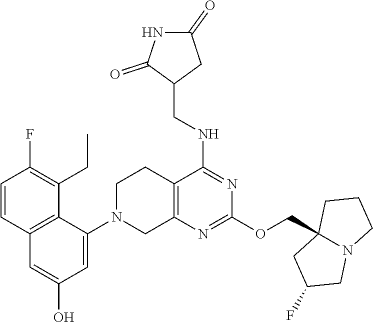 [SMILES: CCc1c(F)ccc2cc(O)cc(N3CCc4c(nc(OC[C@@]56CCCN5C[C@H](F)C6)nc4NCC4CC(=O)NC4=O)C3)c12]

### 3-(((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)amino)methyl)pyrrolidine-2,5-dione

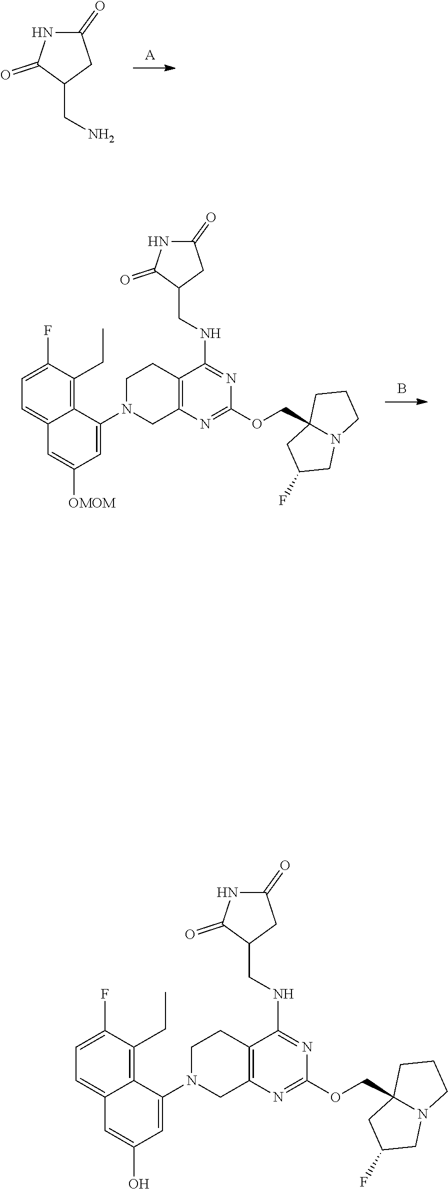

Synthesized according to Example 248 except that K3PO4 instead of N,N-diethylpropan-2-amine was used in step A. The title compound was obtained as brown solid (p-TSA salt). ¹H NMR (400 MHz, METHANOL-d₄) δ=7.73 (d, J=8.4 Hz, 1H), 7.54 (dd, J=5.6, 9.2 Hz, 1H), 7.39 (d, J=8.0 Hz, 1H), 7.17 (t, J=9.6 Hz, 1H), 7.10-7.06 (m, 1H), 7.02 (d, J=2.4 Hz, 1H), 5.67-5.50 (m, 1H), 4.79 (br d, J=5.6 Hz, 2H), 4.08-3.99 (m, 2H), 3.98-3.91 (m, 3H), 3.90-3.80 (m, 2H), 3.63-3.54 (m, 1H), 3.50-3.37 (m, 3H), 3.37-3.33 (m, 2H), 3.16 (d, J=5.6 Hz, 1H), 3.04-2.89 (m, 2H), 2.71-2.65 (m, 2H), 2.63-2.56 (m, 2H), 2.43 (s, 1H), 2.38-2.31 (m, 2H), 2.26-2.17 (m, 1H), 1.08 (t, J=7.2 Hz, 3H); LCMS (ESI, M+1): m/z=607.4.

### Example 415

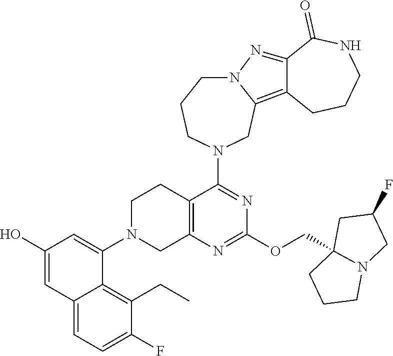 [SMILES: CCc1c(F)ccc2cc(O)cc(N3CCc4c(nc(OC[C@@]56CCCN5C[C@H](F)C6)nc4N4CCCn5nc6c(c5C4)CCCNC6=O)C3)c12]

### 2-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-2,3,4,5,9,10,11,12-octahydroazepino[3′,4′:3,4]pyrazolo[1,5-a][1,4]diazepin-8(1H)-one

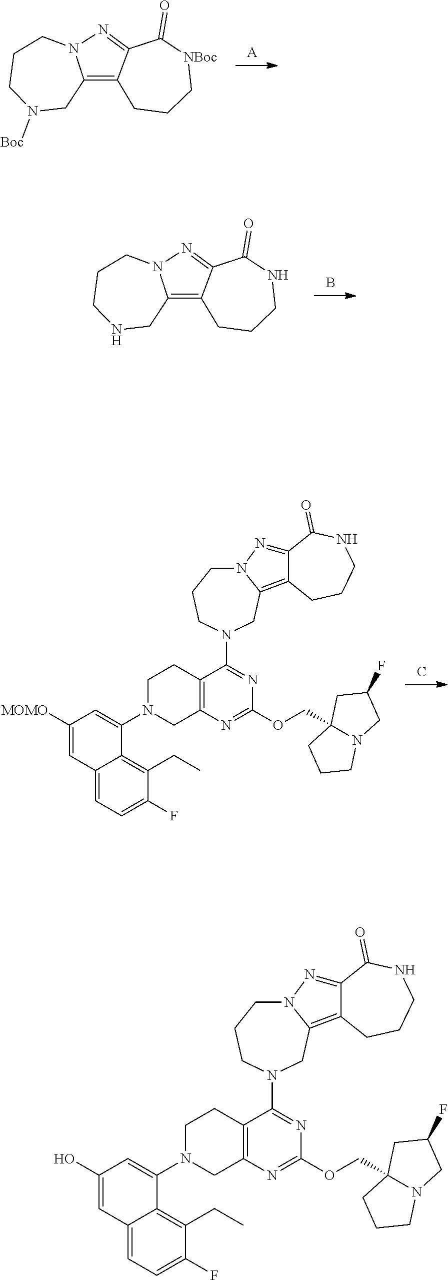

Step A. 2,3,4,5,9,10,11,12-octahydroazepino[3′,4′:3,4]pyrazolo[1,5-a][1,4]diazepin-8(1H)-one: To a solution of ditert-butyl 1 l-oxo-4,8,9,12-tetrazatricyclo[8.5.0.02,8]pentadeca-1,9-diene-4,12-dicarboxylate (200 mg, 1.0 equiv) in MeCN (2.5 mL) was added HCl•dioxane (4 M, 5 mL). The reaction was stirred at 0° C. for 2 hours. The mixture was concentrated to afford the title compound (120 mg, crude) as white solid.

The last two steps were performed according to Example 248. The title compound was obtained as orange solid; ¹H NMR (400 MHz, METHANOL-d₄) δ=7.51 (dd, J=6.0, 8.0 Hz, 1H), 7.14 (t, J=9.6 Hz, 1H), 6.95 (s, 2H), 5.34-5.15 (m, 1H), 4.97 (br dd, J=6.4, 16.4 Hz, 1H), 4.69 (dd, J=4.4, 16.4 Hz, 1H), 4.62-4.44 (m, 2H), 4.15-3.91 (m, 5H), 3.69 (d, J=16.4 Hz, 1H), 3.53-3.33 (m, 4H), 3.24-3.09 (m, 5H), 3.02-2.90 (m, 2H), 2.85-2.73 (m, 1H), 2.73-2.62 (m, 1H), 2.36-1.72 (m, 11H), 1.10 (t, J=7.2 Hz, 3H); LCMS (ESI, M+1): m/z=699.5.

### Example 416

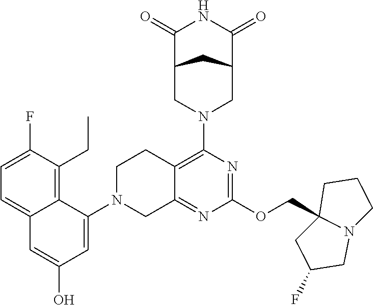 [SMILES: CCc1c(F)ccc2cc(O)cc(N3CCc4c(nc(OC[C@@]56CCCN5C[C@H](F)C6)nc4N4C[C@H]5C[C@@H](C4)C(=O)NC5=O)C3)c12]

### (1R,5S)-7-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-3,7-diazabicyclo[3.3.1]nonane-2,4-dione

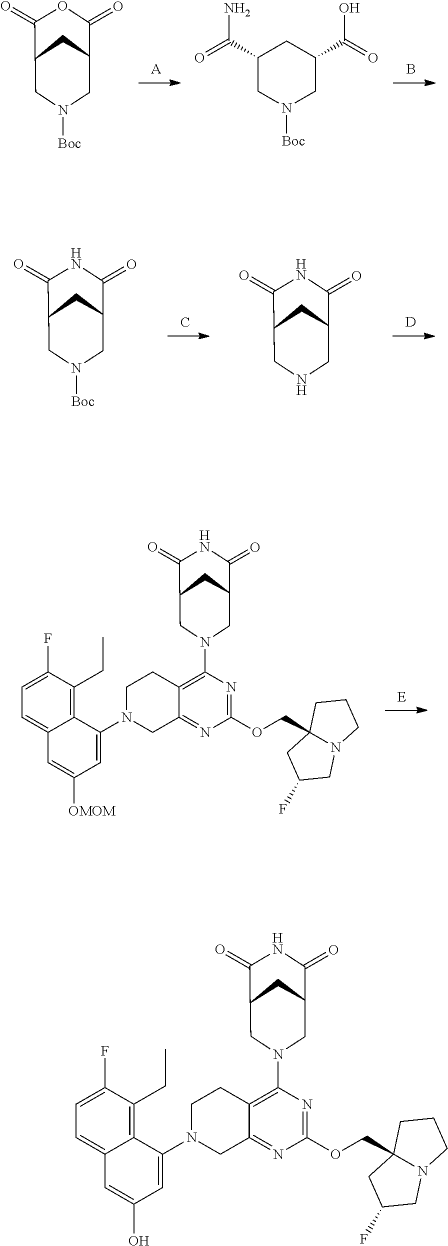

Step A. (3S,5R)-1-(tert-butoxycarbonyl)-5-carbamoylpiperidine-3-carboxylic acid: A solution of tert-butyl (1R,5S)-2,4-dioxo-3-oxa-7-azabicyclo[3.3.1]nonane-7-carboxylate (500 mg, 1.96 mmol, 1.00 eq) in NH₃.H₂O (32.5 g, 260 mmol, 35.7 mL, 28.0% purity, 132 eq) was stirred at 80° C. for 4 hours under N₂ atmosphere. The solvent was removed under reduced pressure to give the crude (3S,5R)-1-(tert-butoxycarbonyl)-5-carbamoylpiperidine-3-carboxylic acid (450 mg, crude) as a white solid.

Step B. tert-butyl (1R,5S)-6,8-dioxo-3,7-diazabicyclo[3.3.1]nonane-3-carboxylate: To a solution of (3S,5R)-1-(tert-butoxycarbonyl)-5-carbamoylpiperidine-3-carboxylic acid (3.30 g, 12.1 mmol, 1.00 eq) in THE (10.0 mL) was added CDI (7.86 g, 48.5 mmol, 4.00 eq). The reaction was stirred at 75° C. for 12 hours under N₂. The mixture was poured into ice water (20 mL), and then 1 N HCl was added until pH was adjusted to 4. The mixture was extracted with DCM (10.0 mL×3). The combined organic layers were dried over Na₂SO₄, filtered and concentrated under reduced pressure to give a residue. The residue was triturated with MTBE (3V) at 25° C. for 30 minutes to give the title compound (mmol, 14.6% yield) as a white solid. 1H NMR: (400 MHz, DMSO) 610.9 (s, 1H), 4.12-4.10 (m, 2H), 3.07-3.04 (m, 2H), 2.62 (s, 2H), 3.30-2.27 (m, 1H), 1.87-1.83 (m, 1H), 1.34 (s, 9H).

Step C. (1R,5S)-3,7-diazabicyclo[3.3.1]nonane-2,4-dione: To a solution of tert-butyl (1R,5S)-6,8-dioxo-3,7-diazabicyclo[3.3.1]nonane-3-carboxylate (250 mg, 983 umol, 1.00 eq) in DCM (2.50 mL) was added TFA (673 mg, 5.90 mmol, 437 L 6.00 eq) at 0° C. The reaction was stirred at 20° C. for 5 hours. The mixture was concentrated in vacuum. The crude product was triturated with EtOAc (10.0 ml) at 25° C. for 40 minutes to afford the title compound (140 mg, 889 umol, 90.4% yield, 98.0% purity) as a white solid. LCMS (ESI, M+1): m/z=155.1 (M+H)⁺.

The last two steps were performed according to Example 248 except that K3PO4 instead of N,N-diethylpropan-2-amine was used in step D. The title compound was obtained as yellow solid (FA salt). ¹H NMR (400 MHz, METHANOL-d₄) δ=7.53-7.49 (m, 1H), 7.17-7.12 (m, 1H), 6.97 (s, 2H), 5.48-5.23 (m, 1H), 4.37-4.26 (m, 2H), 4.15-3.99 (m, 2H), 3.69-3.35 (m, 8H), 3.25 (br s, 1H), 3.14-3.12 (m, 3H), 2.85 (br d, J=9.6 Hz, 2H), 2.59-2.35 (m, 3H), 2.34-2.12 (m, 4H), 2.11-2.00 (m, 3H), 1.11 (br t, J=7.2 Hz, 3H); LCMS (ESI, M+1): m/z=633.4.

### Example 417

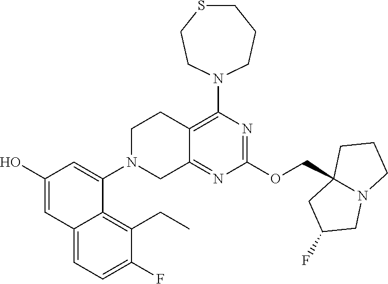 [SMILES: CCc1c(F)ccc2cc(O)cc(N3CCc4c(nc(OC[C@@]56CCCN5C[C@H](F)C6)nc4N4CCCSCC4)C3)c12]

### 5-ethyl-6-fluoro-4-(2-(((2R,7aS)-2-fluorohexahydro-1H-pyrrolizin-7a-yl)methoxy)-4-(1,4-thiazepan-4-yl)-5,6-dihydropyrido[3,4-d]pyrimidin-7(8H)-yl)naphthalen-2-ol

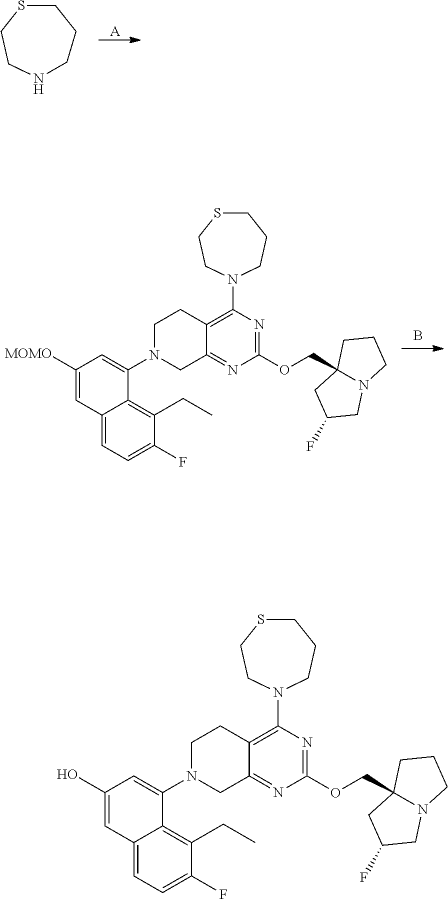

Synthesized according to Example 248. The title compound was obtained as yellow solid (FA salt). ¹H NMR (400 MHz, METHANOL-d₄) δ=7.51 (dd, J=5.6, 8.8 Hz, 1H), 7.14 (t, J=9.2 Hz, 1H), 7.00-6.95 (m, 2H), 5.46-5.29 (m, 1H), 4.37-4.30 (m, 1H), 4.27 (d, J=9.2 Hz, 1H), 4.24-4.19 (m, 1H), 4.19-4.12 (m, 1H), 4.06-3.93 (m, 2H), 3.79-3.71 (m, 1H), 3.68 (d, J=17.6 Hz, 1H), 3.58-3.49 (m, 2H), 3.45 (br d, J=19.2 Hz, 2H), 3.40-3.34 (m, 2H), 3.23-3.11 (m, 4H), 2.84 (td, J=4.4, 14.4 Hz, 1H), 2.79-2.70 (m, 2H), 2.57-2.49 (m, 1H), 2.45-2.25 (m, 2H), 2.19 (br d, J=10.4 Hz, 1H), 2.14-2.06 (m, 2H), 2.06-1.93 (m, 3H), 1.13-1.05 (m, 3H); LCMS (ESI, M+1): m/z=596.1.

### Example 418

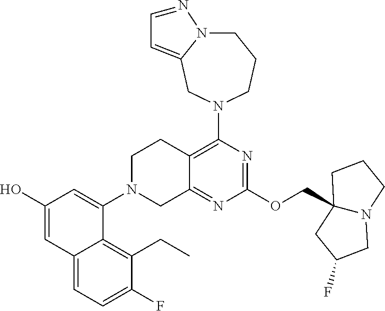 [SMILES: CCc1c(F)ccc2cc(O)cc(N3CCc4c(nc(OC[C@@]56CCCN5C[C@H](F)C6)nc4N4CCCn5nccc5C4)C3)c12]

### 4-(4-(7,8-dihydro-4H-pyrazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

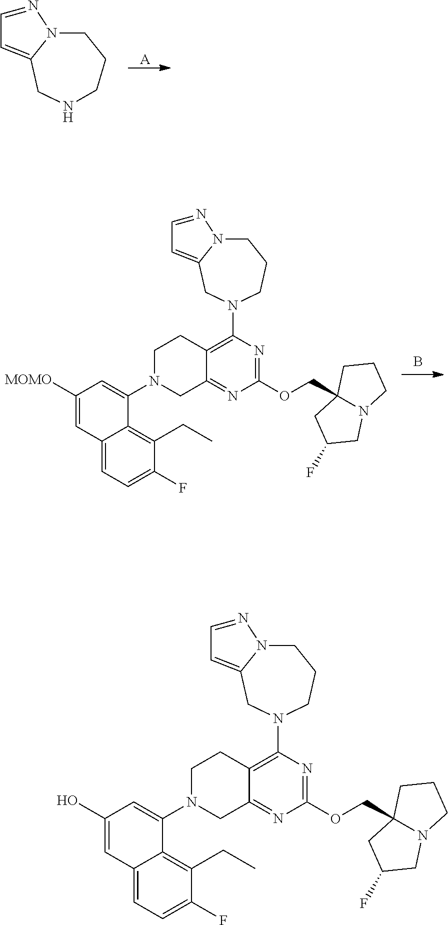

Synthesized according to Example 248. The title compound was obtained as as yellow solid (FA salt). ¹H NMR (400 MHz, METHANOL-d₄) δ=7.50 (dd, J=6.0, 8.8 Hz, 1H), 7.31 (d, J=1.6 Hz, 1H), 7.13 (t, J=9.2 Hz, 1H), 6.96-6.94 (m, 2H), 6.23 (t, J=1.6 Hz, 11H), 5.39 (br d, J=53.2 Hz, 1H), 5.02 (br dd, J=6.4, 16.4 Hz, 1H), 4.82-4.75 (m, 1H), 4.49-4.48 (m, 2H), 4.27-4.21 (m, 3H), 4.06 (br d, J=17.6 Hz, 1H), 3.97 (br dd, J=6.4, 13.6 Hz, 1H), 3.68 (br d, J=17.6 Hz, 1H), 3.62-3.47 (m, 4H), 3.43-3.38 (m, 2H), 3.27-3.18 (m, 3H), 2.82-2.65 (m, 1H), 2.48-1.95 (m, 8H), 1.12-1.08 (m, 3H); LCMS (ESI, M+1): m/z=616.4.

### Example 419

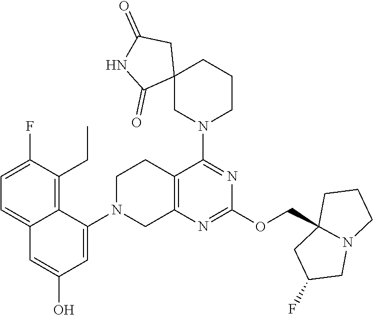 [SMILES: CCc1c(F)ccc2cc(O)cc(N3CCc4c(nc(OC[C@@]56CCCN5C[C@H](F)C6)nc4N4CCCC5(CC(=O)NC5=O)C4)C3)c12]

### 7-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-2,7-diazaspiro[4.5]decane-1,3-dione

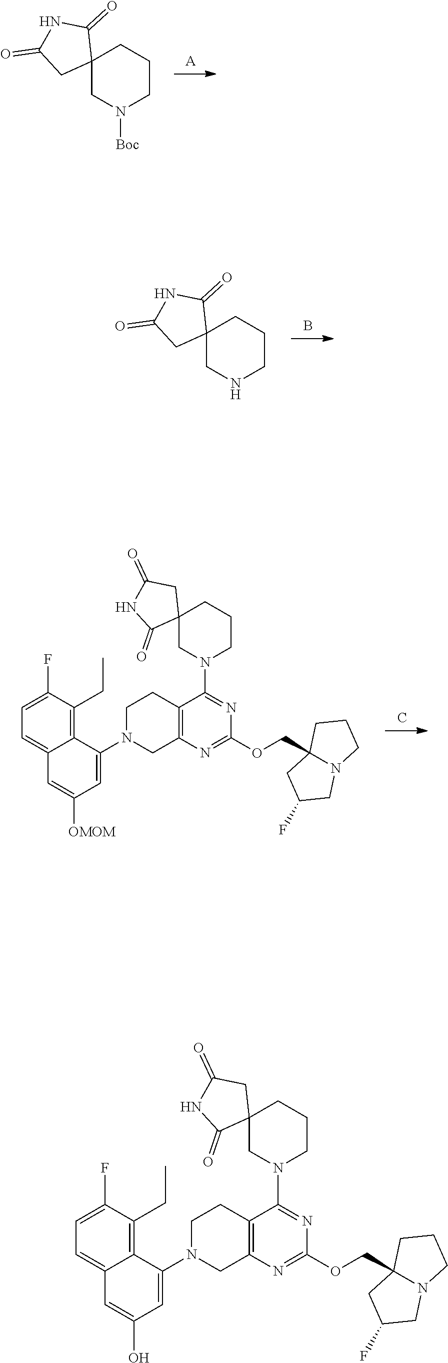

Step A. 2,7-diazaspiro[4.5]decane-1,3-dione: To a mixture of tert-butyl 1,3-dioxo-2,7-diazaspiro[4.5]decane-7-carboxylate (100 mg, 1.0 equiv) in ACN (0.5 mL) was added HCl•dioxane (4M, 1.40 mL, 15 equiv). The reaction was stirred at 20° C. for 0.5 hours. The mixture was concentrated to afford the title compound (70 mg, 92% yield, HCl) as white solid.

The last two steps were performed according to Example 248. The title compound was obtained as yellow solid (FA salt). ¹H NMR (400 MHz, methanol-d₄) δ=7.56-7.45 (m, 1H), 7.20-7.10 (m, 1H), 7.04-6.91 (m, 2H), 5.56-5.32 (m, 1H), 4.42-3.95 (m, 5H), 3.74-3.47 (m, 5H), 3.46-3.32 (m, 4H), 3.27-2.98 (m, 4H), 2.73-2.37 (m, 4H), 2.35-2.00 (m, 5H), 1.97-1.57 (m, 3H), 1.17-1.02 (m, 3H); LCMS (ESI, M+1): m/z=647.2.

### Example 420

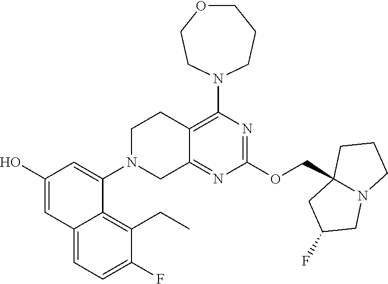 [SMILES: CCc1c(F)ccc2cc(O)cc(N3CCc4c(nc(OC[C@@]56CCCN5C[C@H](F)C6)nc4N4CCCOCC4)C3)c12]

### 5-ethyl-6-fluoro-4-(2-(((2R,7aS)-2-fluorohexahydro-1H-pyrrolizin-7a-yl)methoxy)-4-(1,4-oxazepan-4-yl)-5,6-dihydropyrido[3,4-d]pyrimidin-7(8H)-yl)naphthalen-2-ol

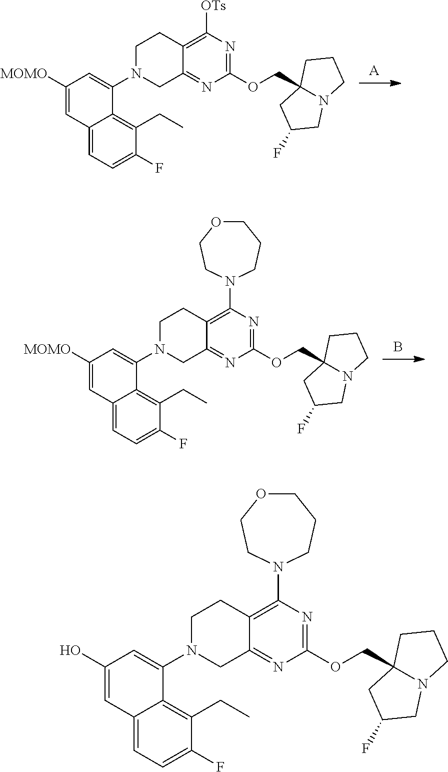

Synthesized according to Example 248. The title compound was obtained as pink solid (FA salt). ¹H NMR (400 MHz, METHANOL-d₄) δ=7.51 (dd, J=6.0, 8.8 Hz, 1H), 7.14 (t, J=9.2 Hz, 1H), 6.97 (br d, J=7.6 Hz, 2H), 5.41-5.19 (m, 1H), 4.23-4.09 (m, 2H), 4.08-3.97 (m, 2H), 3.93 (br dd, J=6.8, 12.8 Hz, 3H), 3.88-3.80 (m, 3H), 3.73 (td, J=4.2, 8.4 Hz, 1H), 3.66 (br d, J=17.6 Hz, 1H), 3.50 (br d, J=6.0 Hz, 1H), 3.44-3.32 (m, 4H), 3.25 (br s, 1H), 3.22-3.12 (m, 2H), 3.10-3.00 (m, 1H), 2.80-2.67 (m, 1H), 2.39-2.24 (m, 1H), 2.24-2.07 (m, 3H), 2.07-1.97 (m, 2H), 1.96-1.84 (m, 2H), 1.10 (br t, J=7.2 Hz, 3H); LCMS (ESI, M+1): m/z=580.3.

### Example 421

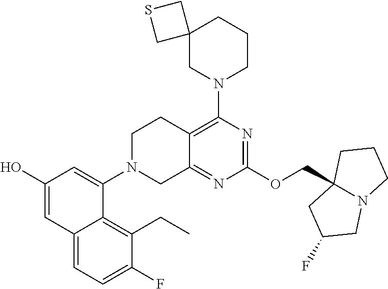 [SMILES: CCc1c(F)ccc2cc(O)cc(N3CCc4c(nc(OC[C@@]56CCCN5C[C@H](F)C6)nc4N4CCCC5(CSC5)C4)C3)c12]

### 5-ethyl-6-fluoro-4-(2-(((2R,7aS)-2-fluorohexahydro-1H-pyrrolizin-7a-yl)methoxy)-4-(2-thia-6-azaspiro[3.5]nonan-6-yl)-5,6-dihydropyrido[3,4-d]pyrimidin-7(8H)-yl)naphthalen-2-ol

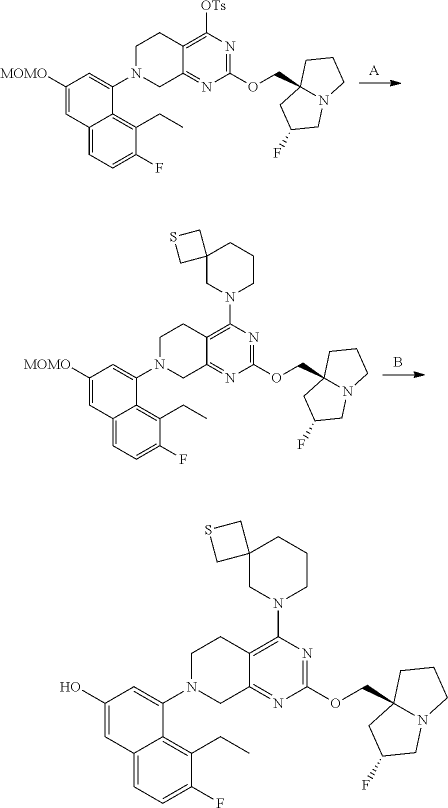

Synthesized according to Example 248. The title compound was obtained as white solid (FA salt). ¹H NMR (400 MHz, METHANOL-d₄) δ=7.51 (dd, J=6.0, 9.2 Hz, 1H), 7.14 (t, J=9.6 Hz, 1H), 7.00-6.94 (m, 2H), 5.39-5.22 (m, 1H), 4.26-4.21 (m, 1H), 4.20-4.11 (m, 2H), 4.10-4.04 (m, 1H), 3.87-3.79 (m, 1H), 3.73-3.68 (m, 1H), 3.67-3.62 (m, 1H), 3.51 (br d, J=8.4 Hz, 1H), 3.45-3.33 (m, 3H), 3.25 (br s, 3H), 3.22-3.13 (m, 2H), 3.08-3.02 (m, 3H), 2.92 (d, J=9.2 Hz, 1H), 2.73-2.66 (m, 2H), 2.39-2.20 (m, 2H), 2.18-2.10 (m, 1H), 2.07-1.99 (m, 2H), 1.97 (br d, J=6.0 Hz, 1H), 1.92 (br d, J=14.4 Hz, 1H), 1.75-1.66 (m, 2H), 1.64-1.55 (m, 1H), 1.12 (t, J=7.2 Hz, 3H); LCMS (ESI, M+1): m/z=622.6.

### Example 422

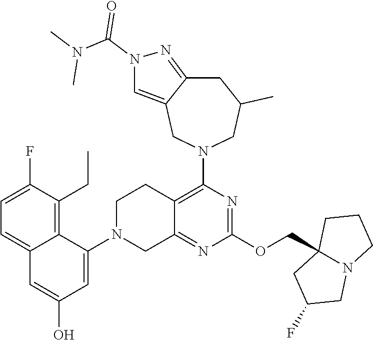 [SMILES: CCc1c(F)ccc2cc(O)cc(N3CCc4c(nc(OC[C@@]56CCCN5C[C@H](F)C6)nc4N4Cc5cn(C(=O)N(C)C)nc5CC(C)C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N,7-trimethyl-5,6,7,8-tetrahydropyrazolo[4,3-c]azepine-2(4H)-carboxamide

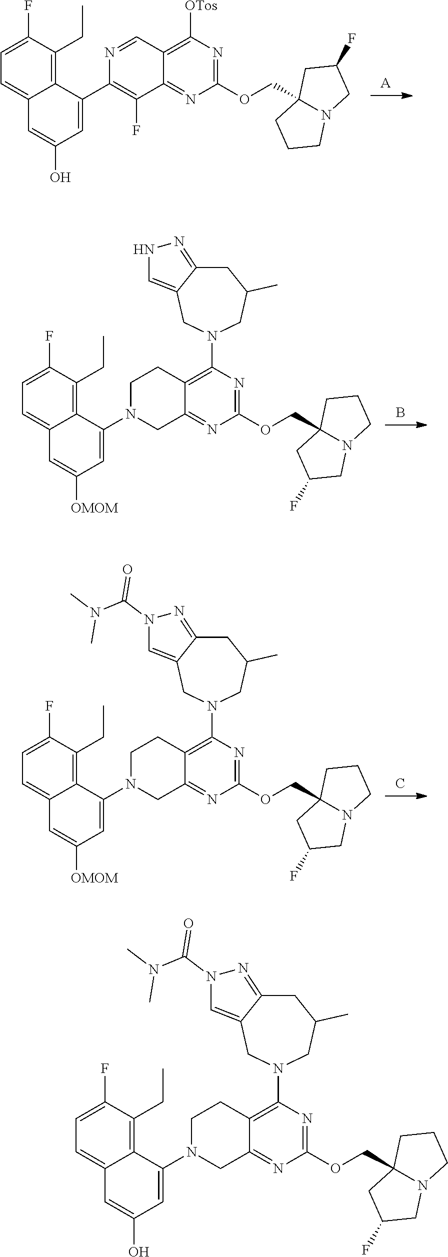

Step A. 5-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d] pyrimidin-4-yl)-7-methyl-2,4,5,6,7,8-hexahydropyrazolo[4,3-c]azepine: To a mixture of 7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl 4-methylbenzenesulfonate (200 mg, 1.0 equiv), 7-methyl-2,4,5,6,7,8-hexahydropyrazolo[4,3-c]azepine (108 mg, 2.0 equiv, HCl), and 4 A molecular sieve (20 mg) in DMF (0.6 mL) was added N,N-diethylpropan-2-amine (260 mg, 7.0 equiv). The mixture was stirred at 40° C. for 16 hours. The mixture was filtered and the filtrate was purified by reversed phase flash chromatography [water (0.10% FA)/acetonitrile] to afford the title compound (110 mg, crude) as a whit solid.

Step B. 5-(7-(8-ethyl-7-fluoro-3-(methoxymethoxy)naphthalen-1-yl)-2-(((2R,7aS)-2-fluorohexahydro-11H-pyrrolizin-7a-yl)methoxy)-5,6,7,8-tetrahydropyrido[3,4-d] pyrimidin-4-yl)-N,N,7-trimethyl-5,6,7,8-tetrahydropyrazolo[4,3-c]azepine-2(4H)-carboxamide: To a solution of 5-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-7-methyl-2,4,5,6,7,8-hexahydropyrazolo[4,3-c]azepine (110 mg, 1.0 equiv) in THF (2 mL) was added NaH (9.79 mg, 60% purity, 1.5 equiv). The mixture was stirred at 0° C. for 0.5 hours. Then N,N-dimethylcarbamoyl chloride (35.1 mg, 2.0 equiv) was added into the mixture and the mixture was stirred at 20° C. for 1 hour. The mixture was quenched with water (50 mL) and extracted with EtOAc (2×20 mL). The combined organic layers were washed with brine (20 mL), dried over anhydrous sodium sulfate, and concentrated to afford the title compound (110 mg, crude) as yellow oil; LCMS (ESI, M+1): m/z=745.6.

Step C. 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N,7-trimethyl-5,6,7,8-tetrahydropyrazolo[4,3-c]azepine-2(4H)-carboxamide: To a solution of 5-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-N,N,7-trimethyl-5,6,7,8-tetrahydropyrazolo[4,3-c]azepine-2(4H)-carboxamide (90 mg, 1.0 equiv) in MeOH (0.5 mL) was added HCl-MeOH (4 M, 1 mL). The reaction was stirred at 0° C. for 1 hour. The mixture was dropped into an ice-cold saturated NaHCO₃ solution (30 mL) and extracted with ethyl acetate (2×10 mL). The combined organic layers were dried over anhydrous sodium sulfate, concentrated, and purified by prep-HPLC [column: Waters Xbridge 150×25 mm x Sum; A: water(NH₄HCO₃), B:ACN, B %: 55%-85% over 8 min] to afford the title compound (44.4 mg, 52% yield) as white solid; ¹H NMR (400 MHz, METHANOL-d₄) δ=8.11-8.05 (m, 1H), 7.51 (dd, J=6.0, 9.0 Hz, 1H), 7.18-7.10 (m, 1H), 7.01-6.91 (m, 2H), 5.33 (br s, 1H), 5.21 (br s, 1H), 4.67-4.48 (m, 2H), 4.28-4.15 (m, 1H), 4.14-3.95 (m, 3H), 3.79-3.35 (m, 7H), 3.18 (br s, 9H), 3.04-2.92 (m, 2H), 2.79-2.64 (m, 2H), 2.40-2.24 (m, 1H), 2.21-1.80 (m, 6H), 1.12-1.04 (m, 4H), 1.02-0.95 (m, 2H); LCMS (ESI, M+1): m/z=701.5.

### Example 423

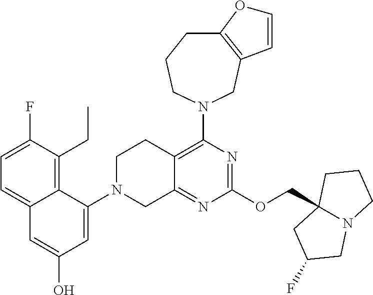 [SMILES: CCc1c(F)ccc2cc(O)cc(N3CCc4c(nc(OC[C@@]56CCCN5C[C@H](F)C6)nc4N4CCCc5occc5C4)C3)c12]

### 4-(4-(7,8-dihydro-4H-furo[3,2-c]azepin-5(6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

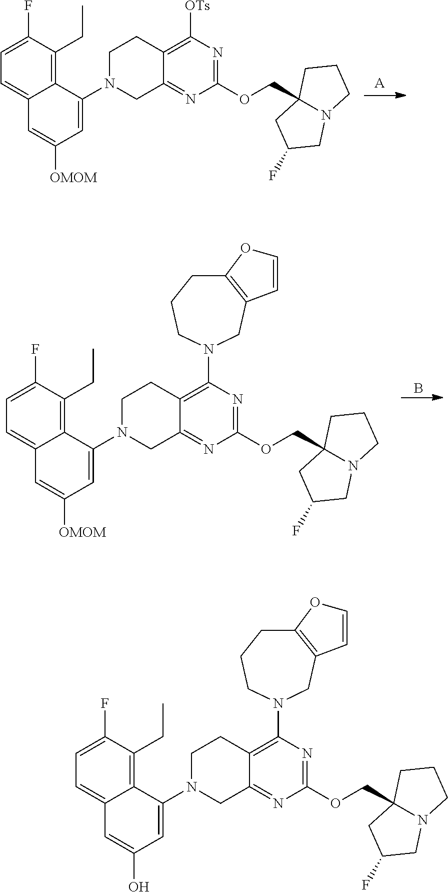

Synthesized according to Example 248. The title compound was obtained as white solid. ¹H NMR (400 MHz, METHANOL-d₄) δ=7.52-7.49 (m, 1H), 7.29-7.21 (m, 1H), 7.14 (t, J=9.4 Hz, 1H), 7.02-6.91 (m, 2H), 6.40-6.39 (m, 1H), 5.34-5.16 (m, 1H), 4.78-4.74 (m, 1H), 4.54-4.49 (m, 1H), 4.24-4.15 (m, 1H), 4.14-4.04 (m, 2H), 4.03-3.98 (m, 1H), 3.93-3.83 (m, 1H), 3.64 (d, J=17.6 Hz, 1H), 3.53-3.45 (m, 1H), 3.42-3.35 (m, 2H), 3.25-3.11 (m, 5H), 3.01-2.95 (m, 1H), 2.92-2.81 (m, 2H), 2.78-2.69 (m, 1H), 2.29-2.17 (m, 1H), 2.16-2.11 (m, 1H), 2.10-2.02 (m, 2H), 2.00-1.81 (m, 4H), 1.12-1.08 (m, 3H); LCMS (ESI, M+1). m/z=616.4.

### Example 424

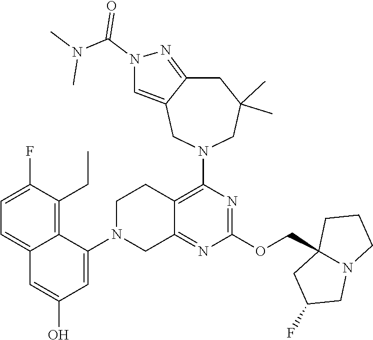 [SMILES: CCc1c(F)ccc2cc(O)cc(N3CCc4c(nc(OC[C@@]56CCCN5C[C@H](F)C6)nc4N4Cc5cn(C(=O)N(C)C)nc5CC(C)(C)C4)C3)c12]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N,7,7-tetramethyl-5,6,7,8-tetrahydropyrazolo[4,3-c]azepine-2(4H)-carboxamide

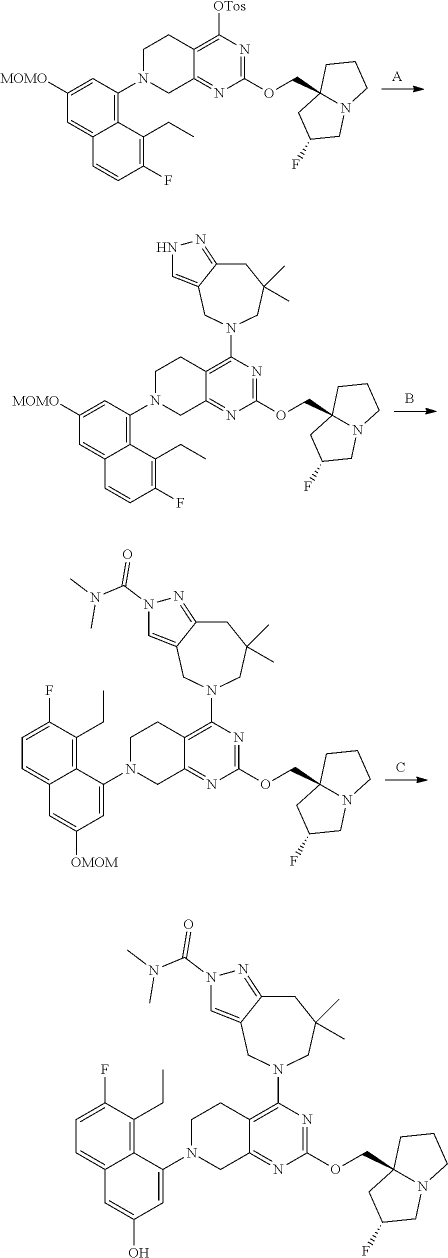

Step A. 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-7,7-dimethyl-2,4,5,6,7,8-hexahydropyrazolo[4,3-c]azepine: To a mixture of 7,7-dimethyl-4,5,6,8-tetrahydro-2H-pyrazolo[4,3-c]azepine (206 mg, 2.0 equiv, 2HCl) in DMF (1 mL) were added 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (300 mg, 1.0 equiv), N,N-diethylpropan-2-amine (391 mg, 7.0 equiv), and 4 A molecular sieve (50 mg). The mixture was stirred at 40° C. for 16 hours. The mixture was concentrated, filtered, and the filtrate was diluted with ethyl acetate (20 mL) and water (50 mL) and extracted with ethyl acetate (20 mL). The combined organic layers were washed with brine (20 mL), dried over anhydrous sodium sulfate, concentrated, and purified by reversed phase flash chromatography [water (0.1% FA)/acetonitrile] to afford the title compound (270 mg, 90% yield) as yellow solid; LCMS (ESI, M+1): m/z=688.5.

Step B. 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-N,N,7,7-tetramethyl-5,6,7,8-tetrahydropyrazolo[4,3-c]azepine-2(4H)-carboxamide: To a solution of 5-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-7,7-dimethyl-2,4,5,6,7,8-hexahydropyrazolo[4,3-c]azepine (230 mg, 1.0 equiv) in THE (5 mL) was added NaH (20.1 mg, 60% purity, 1.5 equiv) at 0° C. The mixture was stirred at 0° C. for 0.5 hours. Then N,N-dimethylcarbamoyl chloride (71.9 mg, 2.0 equiv) was added into the mixture and the mixture was stirred at 20° C. for 1 hour. The mixture was quenched with NH₄Cl saturated solution (20 mL) and extracted with ethyl acetate (2×10 mL). The combined organic layers were dried over anhydrous sodium sulfate and concentrated under vacuum to afford the title compound (275 mg, crude) as yellow oil; LCMS (ESI, M+1): m/z=759.5.

Step C. 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-N,N,7,7-tetramethyl-5,6,7,8-tetrahydropyrazolo[4,3-c]azepine-2(4H)-carboxamide: To a solution of 5-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-N,N,7,7-tetramethyl-5,6,7,8-tetrahydropyrazolo[4,3-c]azepine-2(4H)-carboxamide (250 mg, 1.0 equiv) in MeOH (1.5 mL) was added HCl-MeOH (4 M, 3 mL). The reaction was stirred at 0° C. for 0.5 hours. The mixture was dropped into an ice cold saturated NaHCO₃ solution (60 mL) and was extracted with ethyl acetate (2×20 mL). The combined organic layers were washed with brine (20 mL), dried over anhydrous sodium sulfate, concentrated, purified by prep-HPLC [column: Waters Xbridge 150×25 mm×5 um; A: water (NH₄HCO₃), B: ACN, B %: 56%-86% over 10 min] and lyophilized to afford the title compound (92.3 mg, 38% yield) as yellow solid; ¹H NMR (400 MHz, CHLOROFORM-d) δ=8.04 (d, J=7.2 Hz, 1H), 8.07-8.01 (m, 1H), 7.33 (td, J=6.4, 9.0 Hz, 1H), 7.05 (t, J=9.2 Hz, 1H), 6.90-6.81 (m, 2H), 5.38-5.09 (m, 1H), 4.93 (br t, J=14.8 Hz, 1H), 4.28 (br dd, J=9.6, 14.2 Hz, 1H), 4.21-3.94 (m, 4H), 3.61-3.48 (m, 1H), 3.42-3.22 (m, 8H), 3.19-2.61 (m, 9H), 2.53-2.04 (m, 5H), 2.03-1.81 (m, 3H), 1.13-0.99 (m, 6H), 0.67 (br d, J=4.4 Hz, 3H); LCMS (ESI, M+1): m/z=715.5.

### Example 425

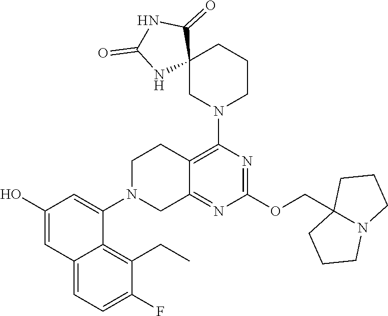 [SMILES: CCc1c(F)ccc2cc(O)cc(N3CCc4c(nc(OCC56CCCN5CCC6)nc4N4CCC[C@]5(C4)NC(=O)NC5=O)C3)c12]

### (R)-7-(7-(8-ethyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione

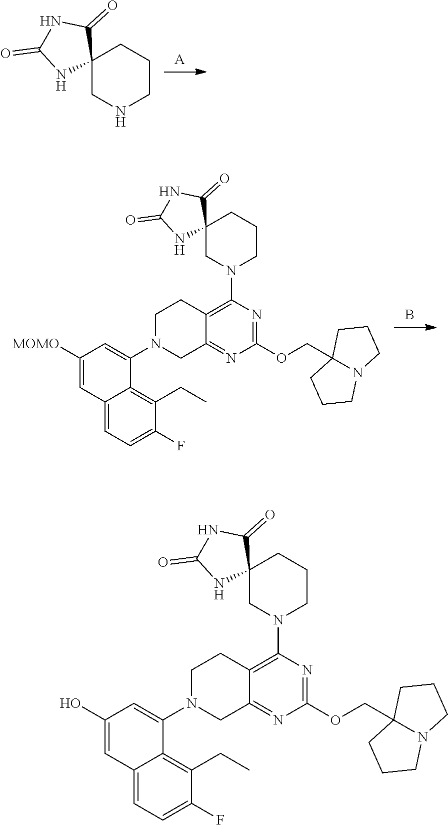

Synthesized according to Example 248. The title compound was obtained as a yellow solid. ¹H NMR (400 MHz, METHANOL-d₄) δ=7.57-7.48 (m, 1H), 7.22-7.10 (m, 1H), 6.98 (br s, 2H), 4.51-4.40 (m, 2H), 4.29-3.94 (m, 4H), 3.66 (br d, J=12.0 Hz, 4H), 3.57-3.44 (m, 3H), 2.92-2.64 (m, 2H), 2.32-1.94 (m, 12H), 1.29 (br s, 2H), 1.11 (br t, J=6.4 Hz, 3H), 0.10 (s, 1H), LCMS (ESI, M+1): m/z=630.4

### Example 426

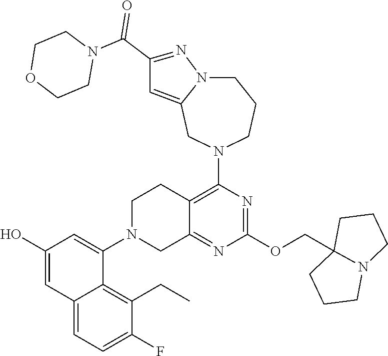 [SMILES: CCc1c(F)ccc2cc(O)cc(N3CCc4c(nc(OCC56CCCN5CCC6)nc4N4CCCn5nc(C(=O)N6CCOCC6)cc5C4)C3)c12]

### (5-(7-(8-ethyl-7-fluoro-3-hydroxynaphthalen-1-yl)-2-((tetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(morpholino)methanone

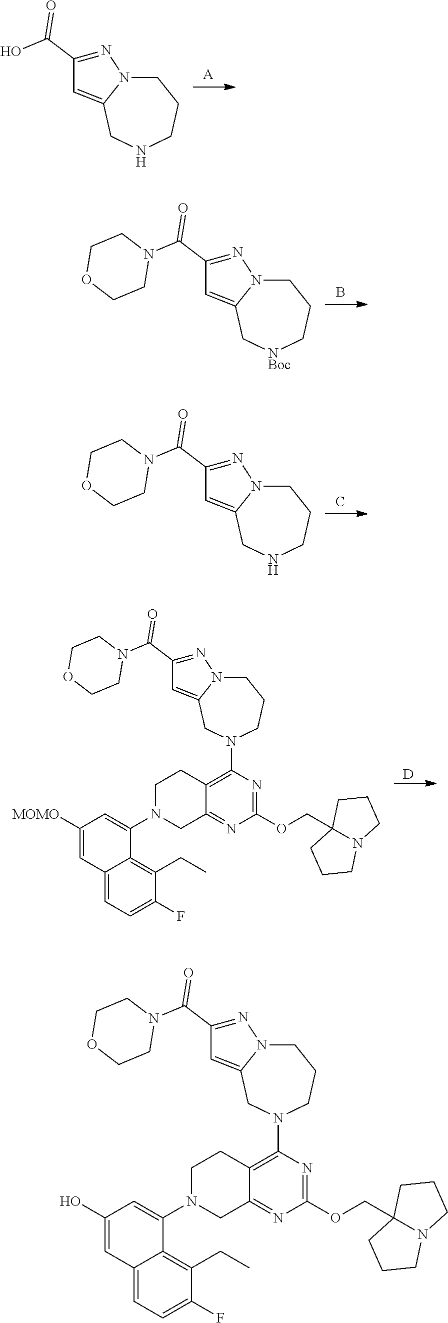

Step A. tert-butyl 2-(morpholine-4-carbonyl)-7,8-dihydro-4H-pyrazolo[1,5-a][1,4]diazepine-5(6H)-carboxylate: To a solution of 5-tert-butoxycarbonyl-4,6,7,8-tetrahydropyrazolo[1,5-a][1,4]diazepine-2-carboxylic acid (300 mg, 1.0 equiv), morpholine (278 mg, 3.0 equiv), and DIEA (1.65 g, 12 equiv) in ethyl acetate (3 mL) was added T3P (2.04 g, 6.0 equiv) at 0° C. The mixture was stirred at 0-25° C. for 2 hours. The mixture was concentrated under reduced pressure and purified by reversed phase flash chromatography [C18, 0.1% formic acid condition] to afford the title compound (320 mg, 85% yield) as white oil; LCMS (ESI, M+1): m/z=351.3.

Step B. morpholino(5,6,7,8-tetrahydro-4H-pyrazolo[1,5-a][1,4]diazepin-2-1llmethanone: To a solution of tert-butyl 2-(morpholine-4-carbonyl)-4,6,7,8-tetrahydropyrazolo[1,5-a][1,4]diazepine-5-carboxylate (310 mg, 1.0 equiv) in MeCN (3 mL) was added HCl•dioxane (4 M, 6.8 equiv) at 0° C. The mixture was stirred at 0-25° C. for 0.5 hours. The mixture was concentrated under reduced pressure to afford the title compound (215 mg, crude) as white solid.

The last two steps were performed according to Example 248. The title compound was obtained as pink solid (FA salt). ¹H NMR (400 MHz, DMSO-d₆) δ=8.23 (s, 1H), 7.64-7.44 (m, 1H), 7.29-7.09 (m, 1H), 6.99 (s, 2H), 6.57 (s, 1H), 5.02 (br d, J=16.4 Hz, 1H), 4.76 (br d, J=16.4 Hz, 1H), 4.56-4.44 (m, 2H), 4.21-4.13 (m, 1H), 4.01-3.92 (m, 4H), 3.88 (br d, J=17.6 Hz, 2H), 3.59 (br s, 9H), 3.48-3.41 (m, 2H), 3.16-3.00 (m, 4H), 2.73-2.63 (m, 3H), 2.34-2.10 (m, 2H), 1.90-1.76 (m, 5H), 1.62 (br dd, J=6.8, 11.9 Hz, 2H), 1.10-1.04 (m, 3H), LCMS (ESI, M+1): m/z =711.5.

### Example 427

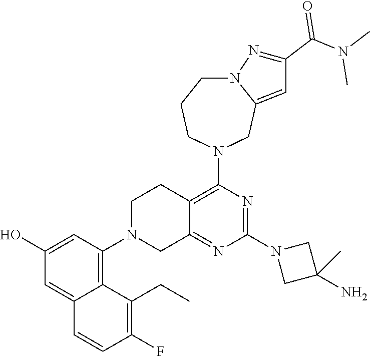 [SMILES: CCc1c(F)ccc2cc(O)cc(N3CCc4c(nc(N5CC(C)(N)C5)nc4N4CCCn5nc(C(=O)N(C)C)cc5C4)C3)c12]

### 5-(2-(3-amino-3-methylazetidin-1-yl)-7-(8-ethyl-7-fluoro-3-hydroxynaphthalen-1-yl)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide

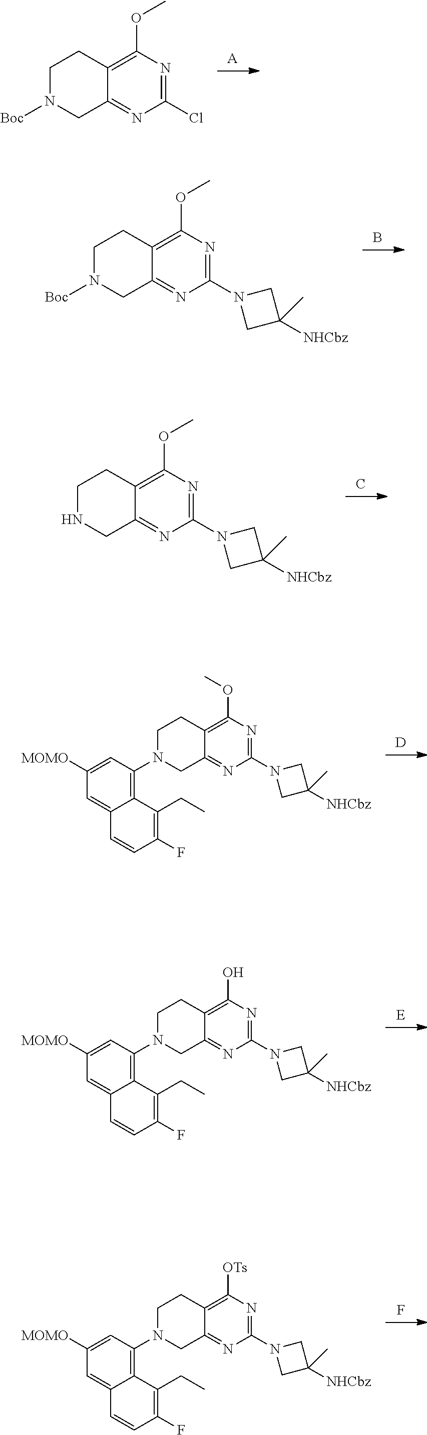

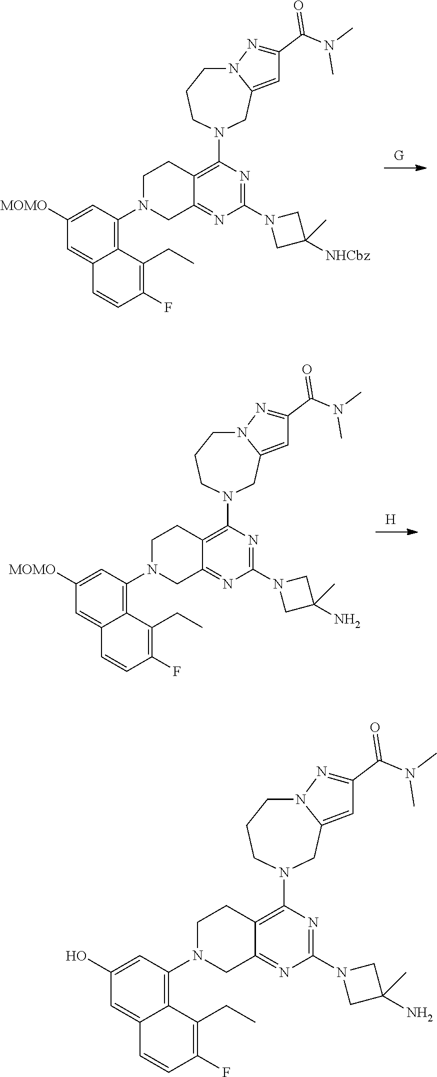

Step A. tert-butyl 2-(3-(((benzyloxy)carbonyl)amino)-3-methylazetidin-1-yl)-4-methoxy-5,6-dihydropyrido[3,4-d]pyrimidine-7(8H)-carboxylate: To a mixture of tert-butyl 2-chloro-4-methoxy-6,8-dihydro-5H-pyrido[3,4-d]pyrimidine-7-carboxylate (5.1 g, 1 equiv), benzyl N-(3-methylazetidin-3-yl)carbamate (4.50 g, 1.2 equiv) in toluene (50 mL) were added Pd(OAc)₂ (382 mg, 0.1 equiv), BINAP (2.12 g, 3.40 mmol, 0.2 equiv), and Cs₂CO3 (16.6 g, 3 equiv). The mixture was degassed and purged with nitrogen 3 times. The reaction was stirred at 110° C. for 2 hours under nitrogen atmosphere. The mixture was diluted with water (200 mL) and extracted with ethyl acetate (3×100 mL). The combined organic layers were dried over anhydrous sodium sulfate, concentrated, and purified by silica gel chromatography (petroleum ether/ethyl acetate 5/1 to ethyl acetate) to afford the title compound (12 g, 66% yield,) as yellow oil; ¹H NMR (400 MHz, chloroform-d) δ=7.41-7.29 (m, 5H), 5.13-5.08 (m, 2H), 5.06-4.98 (m, 1H), 4.40-4.35 (m, 2H), 4.28-4.18 (m, 2H), 3.97 (br d, J=8.8 Hz, 2H), 3.91-3.86 (m, 3H), 3.60 (br t, J=5.6 Hz, 2H), 2.56-2.48 (m, 2H), 1.67-1.63 (m, 3H), 1.48 (s, 9H); LCMS (ESI, M+1): m/z=484.4.

Step B. benzyl (1-(4-methoxy-5,6,7,8-tetrahydropyrido[3,4-d]pyrimidin-2-yl)-3-methylazetidin-3-yl)carbamate: To a solution of tert-butyl 2-[3-(benzyloxycarbonylamino)-3-methyl-azetidin-1-yl]-4-methoxy-6,8-dihydro-5H-pyrido[3,4-d]pyrimidine-7-carboxylate (13.2 g, 1 equiv) in ACN (50 mL) was added HCl-MeOH (4 M, 103 mL, 15 equiv). The reaction was stirred at 20° C. for 1 hour. The mixture was concentrated and diluted with water (70 mL). Solid NaOH was added until pH=10 in ice-water bath. The aqueous phase was extracted with dichloromethane (6×120 mL). Combined organic layers were dried over anhydrous sodium sulfate and concentrated to afford the title compound (10.5 g, crude) as a yellow solid.

Step C. benzyl (1-(7-(8-ethyl-7-fluoro-3-(methoxymethoxy)naphthalen-1-yl)-4-methoxy-5,6,7,8-tetrahydropyrido[3,4-d]pyrimidin-2-yl)-3-methylazetidin-3-yl)carbamate: To a mixture of benzyl N-[1-(4-methoxy-5,6,7,8-tetrahydropyrido[3,4-d]pyrimidin-2-yl)-3-methyl-azetidin-3-yl]carbamate (9 g, 1 equiv) and [8-ethyl-7-fluoro-3-(methoxymethoxy)-1-naphthyl]trifluoromethanesulfonate (10.77 g, 28.17 mmol, 1.2 equiv) in toluene (135 mL) were added Pd₂(dba)₃ (2.15 g, 0.1 equiv), Xantphos (2.72 g, 0.2 equiv), and Cs₂CO₃ (19.1 g, 2.5 equiv). The reaction was degassed and purged with nitrogen 3 times. The mixture was stirred at 110° C. for 14 hours under nitrogen atmosphere. The mixture was filtered and purified by chromatography (Al₂O₃, petroleum ether/ethyl acetate 100/1 to ethyl acetate) followed by reversed phase flash chromatography [C18, 0.1% formic acid condition] to afford the title compound (4.5 g, 25% yield) as brown solid; ¹H NMR (400 MHz, dimethylsulfoxide-d₆) δ=7.87-7.79 (m, 1H), 7.72 (dd, J=6.0, 8.8 Hz, 1H), 7.37-7.27 (m, 7H), 7.15 (d, J=2.4 Hz, 1H), 5.32-5.26 (m, 2H), 5.03 (s, 2H), 4.06 (s, 2H), 3.90 (s, 3H), 3.84-3.64 (m, 4H), 3.46-3.42 (m, 3H), 3.40-3.36 (m, 1H), 3.27-3.11 (m, 3H), 2.79-2.66 (m, 1H), 2.62-2.54 (m, 1H), 1.52-1.45 (m, 3H), 1.19-1.14 (m, 3H).

Step D. benzyl (1-(7-(8-ethyl-7-fluoro-3-(methoxymethoxy)naphthalen-1-yl)-4-hydroxy-5,6,7,8-tetrahydropyrido[3,4-d]pyrimidin-2-yl)-3-methylazetidin-3-yl)carbamate: To a solution of NaH (342 mg, 60% purity, 2 equiv) in DMAC (26 mL) was added EtSH (1.02 g, 3.84 equiv) at 20° C. The mixture was stirred at 20° C. for 0.5 hours. Benzyl (1-(7-(8-ethyl-7-fluoro-3-(methoxymethoxy)naphthalen-1-yl)-4-methoxy-5,6,7,8-tetrahydropyrido[3,4-d]pyrimidin-2-yl)-3-methylazetidin-3-yl)carbamate (2.63 g, 1 equiv) was added to the above mixture. The reaction was stirred at 60° C. for 1 hour. The mixture was diluted with water (60 mL) at 0° C. and extracted with ethyl acetate (3×30 mL). The combined organic layers were washed with brine (20 mL×3), dried over anhydrous sodium sulfate, filtered, and concentrated to afford the title compound (2.3 g, crude) as a brown oil; LCMS (ESI, M+1): m/z=602.3.

Step E. 2-(3-(((benzyloxy)carbonyl)amino)-3-methylazetidin-1-yl)-7-(8-ethyl-7-fluoro-3-(methoxymethoxy)naphthalen-1-yl)-5,6,7,8-tetrahydropyrido[3,4-d]pyrimidin-4-yl 4-methylbenzenesulfonate: To a solution of benzyl N-[1-[7-[8-ethyl-7-fluoro-3-(methoxymethoxy)-1-naphthyl]-4-hydroxy-6,8-dihydro-5H-pyrido[3,4-d]pyrimidin-2-yl]-3-methyl-azetidin-3-yl]carbamate (2.3 g, 71% purity, 1 equiv), N,N-diethylpropan-2-amine (1.05 g, 3.0 equiv), and DMAP (33.2 mg, 0.1 equiv) in DCM (50 mL) was added TosCl (776 mg, 1.5 equiv) at 0° C. The reaction was stirred at 20° C. for 2 hours. The mixture was diluted with water (50 mL) and the layers were separated. The aqueous phase was extracted with ethyl acetate (2×40 mL). The combined organic layers were dried over anhydrous sodium sulfate, filtered, concentrated, and purified by silica gel chromatography (petroleum ether/ethyl acetate 10/1 to 0/1) to afford the title compound (2 g, 93% yield) as a yellow solid; LCMS (ESI, M+1): m/z=756.3.

Step F. benzyl (1-(4-(2-(dimethylcarbamoyl)-7,8-dihydro-4H-pyrazolo[1,5-a][1,4]diazepin-5(6H)-yl)-7-(8-ethyl-7-fluoro-3-(methoxymethoxy)naphthalen-1-yl)-5,6,7,8-tetrahydropyrido[3,4-d]pyrimidin-2-yl)-3-methylazetidin-3-yl)carbamate: To a mixture of 2-(3-(((benzyloxy)carbonyl)amino)-3-methylazetidin-1-yl)-7-(8-ethyl-7-fluoro-3-(methoxymethoxy)naphthalen-1-yl)-5,6,7,8-tetrahydropyrido[3,4-d]pyrimidin-4-yl 4-methylbenzenesulfonate (150 mg, 1.0 equiv) in DMF (0.1 mL) were added N,N-diethylpropan-2-amine (513 mg, 20 equiv), N,N-dimethyl-5,6,7,8-tetrahydro-4H-pyrazolo[1,5-a][1,4]diazepine-2-carboxamide (62.0 mg, 1.5 equiv), and 4A molecular sieve (70 mg). The reaction was stirred at 60° C. for 72 hours. The mixture was filtered and purified by prep-HPLC [Phenomenex luna C18 150 x 25 mm×10 μm; A: water (FA), B: ACN; B %: 32%-62% over 58 min] to afford the title compound (100 mg, 62% yield) as yellow solid; LCMS (ESI, M+1): m/z=792.6.

Step G. 5-(2-(3-amino-3-methylazetidin-1-yl)-7-(8-ethyl-7-fluoro-3-(methoxymethoxy)naphthalen-1-yl)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide: To a mixture of benzyl (1-(4-(2-(dimethylcarbamoyl)-7,8-dihydro-4H-pyrazolo[1,5-a][1,4]diazepin-5(6H)-yl)-7-(8-ethyl-7-fluoro-3-(methoxymethoxy)naphthalen-1-yl)-5,6,7,8-tetrahydropyrido[3,4-d]pyrimidin-2-yl)-3-methylazetidin-3-yl)carbamate (100 mg, 1.0 equiv) in MeOH (2 mL) were added NH₃MeOH (0.04 mL, 20% purity) and Pd/C (20.0 mg, 10% purity) under nitrogen atmosphere. The suspension was degassed and purged with hydrogen 3 times. The reaction was stirred under hydrogen (50 psi) at 40° C. for 2 hours. The mixture was filtered and concentrated to afford the title compound (58.0 mg, crude) as yellow oil.

Step H. 5-(2-(3-amino-3-methylazetidin-1-yl)-7-(8-ethyl-7-fluoro-3-hydroxynaphthalen-1-yl)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide: To a mixture of 5-(2-(3-amino-3-methylazetidin-1-yl)-7-(8-ethyl-7-fluoro-3-(methoxymethoxy)naphthalen-1-yl)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide (58.0 mg, 1.0 equiv) in ACN (165 μL) was added HCl-MeOH (4 M, 330 μL, 15 equiv). The reaction was stirred at 20° C. for 0.5 hours. The mixture was concentrated, dissolved in methanol (1 mL), neutralized with solid NaHCO₃, filtered, purified by prep-HPLC [Welch Ultimate C 18×150×25 mm×5 μm; A: water (FA), B:ACN; B %: 9%-39% over 10 min] and lyophilized to afford the title compound (23.5 mg, 40% yield, 0.64FA) as yellow solid (FA salt). ¹H NMR (400 MHz, methanol-d₄) δ=7.54-7.47 (m, 1H), 7.18-7.10 (m, 1H), 6.98-6.92 (m, 2H), 6.56-6.50 (m, 1H), 4.96-4.90 (m, 1H), 4.79-4.73 (m, 1H), 4.58-4.46 (m, 2H), 4.16-4.08 (m, 1H), 4.07-4.01 (m, 1H), 4.00-3.90 (m, 5H), 3.63-3.55 (m, 1H), 3.53-3.47 (m, 1H), 3.44-3.35 (m, 2H), 3.35-3.32 (m, 3H), 3.22-3.12 (m, 2H), 3.11-3.05 (m, 3H), 2.70-2.60 (m, 1H), 2.35-2.23 (m, 1H), 2.05-1.94 (m, 1H), 1.59-1.50 (m, 3H), 1.15-1.07 (m, 3H); 19F NMR (400 MHz, methanol-d₄) δ=−123.052; LCMS (ESI, M+1): m/z=614.5.

### Example 428

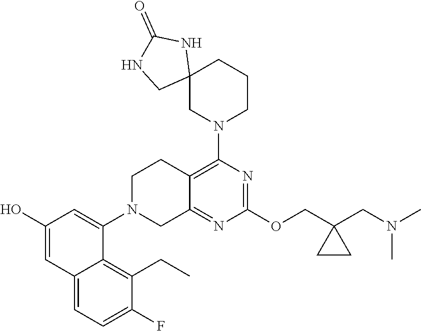 [SMILES: CCc1c(F)ccc2cc(O)cc(N3CCc4c(nc(OCC5(CN(C)C)CC5)nc4N4CCCC5(CNC(=O)N5)C4)C3)c12]

### 7-(2-((1-((dimethylamino)methyl)cyclopropyl)methoxy)-7-(8-ethyl-7-fluoro-3-hydroxynaphthalen-1-yl)-5,6,7,8-tetrahydropyrido[3,4-d]pyrimidin-4-yl)-1,3,7-triazaspiro[4.5]decan-2-one

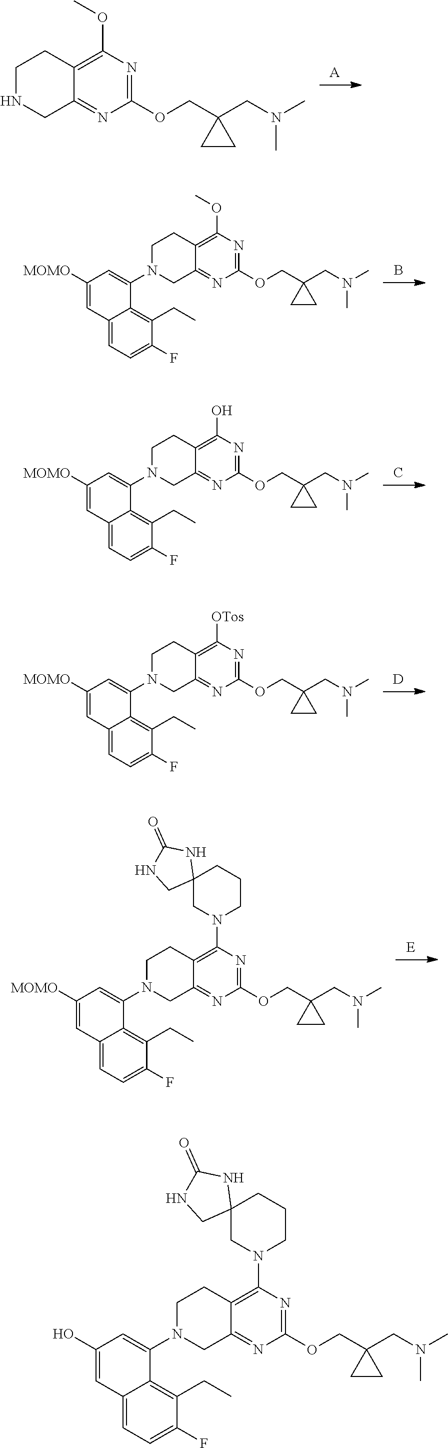

Step A. 1-(1-(((7-(8-ethyl-7-fluoro-3-(methoxymethoxy)naphthalen-1-yl)-4-methoxy-5,6,7,8-tetrahydropyrido[3,4-d]pyrimidin-2-yl)oxy)methyl)cyclopropyl)-N,N-dimethylmethanamine: To a mixture of 1-(1-(((4-methoxy-5,6,7,8-tetrahydropyrido[3,4-d]pyrimidin-2-yl)oxy)methyl)cyclopropyl)-N,N-dimethylmethanamine (0.7 g, 1.0 equiv) and 8-ethyl-7-fluoro-3-(methoxymethoxy)naphthalen-1-yl trifluoromethanesulfonate (915 mg, 1.0 equiv) in toluene (10 mL) was added Pd₂(dba)₃ (219 mg, 0.1 equiv), Cs₂CO₃ (2.34 g, 3.0 equiv), and Xantphos (693 mg, 0.5 equiv). The mixture was degassed and purged with nitrogen 3 times. The reaction was stirred at 110° C. for 12 hours. The mixture was diluted with water (50 mL) and extracted with ethyl acetate (3×30 mL). The combined organic layers were washed with brine (100 mL), dried over anhydrous sodium sulfate, filtered, concentrated, and purified by reversed phase chromatography [water (0.1% formic acid)/acetonitrile)] to afford the title compound (0.5 g, 40% yield) as yellow solid; LCMS (ESI, M+1): m/z=525.2.

Step B. 2-((1-((dimethylamino)methyl)cyclopropyl)methoxy)-7-(8-ethyl-7-fluoro-3-(methoxymethoxy)naphthalen-1-yl)-5,6,7,8-tetrahydropyrido[3,4-d]pyrimidin-4-ol: To a solution of EtSH (189 mg, 4.0 equiv) in DMAC (4 mL) was added NaH (61.0 mg, 60% purity, 2.0 equiv) at 10° C. The reaction was stirred at 10° C. for 0.5 hours. 1-(1-(((7-(8-ethyl-7-fluoro-3-(methoxymethoxy)naphthalen-1-yl)-4-methoxy-5,6,7,8-tetrahydropyrido[3,4-d]pyrimidin-2-yl)oxy)methyl)cyclopropyl)-N,N-dimethylmethanamine (0.4 g, 1.0 equiv) was added to the resulting mixture. The reaction was stirred at 60° C. for 1 hour. The reaction was diluted with water (20 mL) and extracted with ethyl acetate (3×50 mL). The combined organic layers were washed with brine (50 mL×3), dried over anhydrous sodium sulfate, filtered, and concentrated to afford the title compound (380 mg, 98% yield) as white solid; LCMS (ESI, M+1): m/z=511.3.

Step C. 2-((1-((dimethylamino)methyl)cyclopropyl)methoxy)-7-(8-ethyl-7-fluoro-3-(methoxymethoxy)naphthalen-1-yl)-5,6,7,8-tetrahydropyrido[3,4-d]pyrimidin-4-yl 4-methylbenzenesulfonate: To a mixture of 2-((1-((dimethylamino)methyl)cyclopropyl)methoxy)-7-(8-ethyl-7-fluoro-3-(methoxymethoxy)naphthalen-1-yl)-5,6,7,8-tetrahydropyrido[3,4-d]pyrimidin-4-ol (0.38 g, 1.0 equiv), N,N-diethylpropan-2-amine (289 mg, 3.0 equiv), and DMAP (9.09 mg, 0.1 equiv) in DCM (4 mL) was added 4-methylbenzene-1-sulfonyl chloride (213 mg, 1.5 equiv) at 0° C. The reaction was stirred at 20° C. for 2 hours. The mixture was diluted with water (20 mL) and extracted with DCM (3×100 mL). The combined organic layers were washed with brine (50 mL), dried over anhydrous sodium sulfate, filtered, concentrated, and purified by reversed phase chromatography [C18, 0.1% formic acid condition] to afford the title compound (350 mg, 71% yield) as white solid; ¹H NMR (400 MHz, dimethylsulfoxide-d₆) δ=8.03-7.97 (m, 2H), 7.76-7.71 (m, 1H), 7.56-7.49 (m, 2H), 7.36-7.29 (m, 2H), 7.20 (d, J=2.4 Hz, 1H), 5.30 (s, 2H), 3.96-3.79 (m, 4H), 3.42 (s, 3H), 3.28-3.11 (m, 4H), 2.94-2.79 (m, 1H), 2.78-2.66 (m, 1H), 2.46-2.42 (m, 3H), 2.20-2.14 (m, 2H), 2.13 (s, 6H), 0.97 (t, J=7.2 Hz, 3H), 0.60-0.47 (m, 2H), 0.38 (s, 2H); LCMS (ESI, M+1): m/z=665.1.

Step D. 7-(2-((1-((dimethylamino)methyl)cyclopropyl)methoxy)-7-(8-ethyl-7-fluoro-3-(methoxymethoxy)naphthalen-1-yl)-5,6,7,8-tetrahydropyrido[3,4-d] pyrimidin-4-yl)-1,3,7-triazaspiro[4.5]decan-2-one: To a mixture of 2-((1-((dimethylamino)methyl)cyclopropyl)methoxy)-7-(8-ethyl-7-fluoro-3-(methoxymethoxy)naphthalen-1-yl)-5,6,7,8-tetrahydropyrido[3,4-d]pyrimidin-4-yl 4-methylbenzenesulfonate (100 mg, 1.0 equiv) and 1,3,7-triazaspiro[4.5]decan-2-one (46.7 mg, 2.0 equiv) in DMF (0.5 mL) were added N,N-diethylpropan-2-amine (58.3 mg, 3.0 equiv) and 4A molecular sieve (50 mg, 1.0 equiv). The reaction was stirred at 40° C. for 12 hours. The mixture was filtered, purified by prep-HPLC [Welch Xtimate C18 150×25 mm×5 μm; A: water (TFA), B: ACN; B %: 21%-51% over 10 min] and lyophilized to afford the title compound (60.0 mg, 52% yield) as yellow solid; ¹H NMR (400 MHz, chloroform-d) δ=7.60-7.51 (m, 1H), 7.26-7.15 (m, 2H), 7.10-6.95 (m, 1H), 5.35-5.16 (m, 2H), 5.06-4.69 (m, 1H), 4.42-4.13 (m, 2H), 3.94-3.67 (m, 2H), 3.60-3.51 (m, 3H), 3.50-3.41 (m, 2H), 3.40-3.29 (m, 3H), 3.25-3.04 (m, 4H), 3.01-2.86 (m, 7H), 2.31-2.09 (m, 2H), 2.08-1.67 (m, 4H), 1.47-1.37 (m, 1H), 1.20-1.04 (m, 3H), 1.03-0.85 (m, 2H), 0.84-0.66 (m, 2H).

Step E. 7-(2-((1-((dimethylamino)methyl)cyclopropyl)methoxy)-7-(8-ethyl-7-fluoro-3-hydroxynaphthalen-1-yl)-5,6,7,8-tetrahydropyrido[3,4-d] pyrimidin-4-yl)-1,3,7-triazaspiro[4.5]decan-2-one: To a mixture of 7-(2-((1-((dimethylamino)methyl)cyclopropyl)methoxy)-7-(8-ethyl-7-fluoro-3-(methoxymethoxy)naphthalen-1-yl)-5,6,7,8-tetrahydropyrido[3,4-d]pyrimidin-4-yl)-1,3,7-triazaspiro[4.5]decan-2-one (50.0 mg, 1.0 equiv) in ACN (150 μL) was added HCl•MeOH (4M, 289 μL, 15 equiv). The reaction was stirred at 20° C. for 1 hour. The mixture was concentrated, dissolved in methanol (1 mL), neutralized with solid NaHCO₃, filtered, purified by prep-HPLC [Phenomenex luna C18 150×25 mm×10 μm; A: water (FA), B: ACN; B %: 16%-46% over 10 min] and lyophilized to afford the title compound (0.91 mg, 1.7% yield, 1.24FA) as brown solid (FA salt). ¹H NMR (400 MHz, methanol-d₄) δ=7.56-7.48 (m, 1H), 7.19-7.11 (m, 1H), 7.03-6.95 (m, 2H), 4.61-4.57 (m, 2H), 4.33-4.17 (m, 2H), 4.16-4.05 (m, 1H), 3.76-3.65 (m, 2H), 3.64-3.60 (m, 1H), 3.60-3.47 (m, 3H), 3.45-3.40 (m, 2H), 3.20-3.03 (m, 4H), 2.87-2.76 (m, 6H), 2.75-2.67 (m, 1H), 2.00-1.72 (m, 4H), 1.16-1.07 (m, 3H), 0.90-0.83 (m, 2H), 0.78-0.71 (m, 2H); ¹⁹F NMR (400 MHz, methanol-d₄) δ=−123.052; LCMS (ESI, M+1): m/z=604.3.

### Example 429

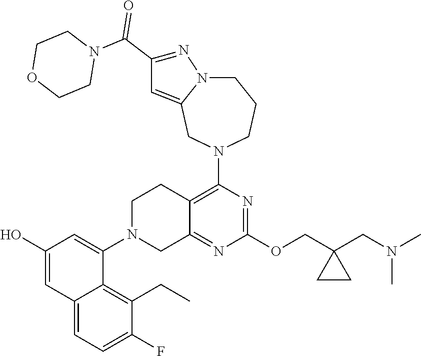 [SMILES: CCc1c(F)ccc2cc(O)cc(N3CCc4c(nc(OCC5(CN(C)C)CC5)nc4N4CCCn5nc(C(=O)N6CCOCC6)cc5C4)C3)c12]

### (5-(2-((1-((dimethylamino)methyl)cyclopropyl)methoxy)-7-(8-ethyl-7-fluoro-3-hydroxynaphthalen-1-yl)-5,6,7,8-tetrahydropyrido[3,4-d]pyrimidin-4-yl)-5,6,7,8-tetrahydro-4H-pyrazolo[1,5-a][1,4]diazepin-2-yl)(morpholino)methanone

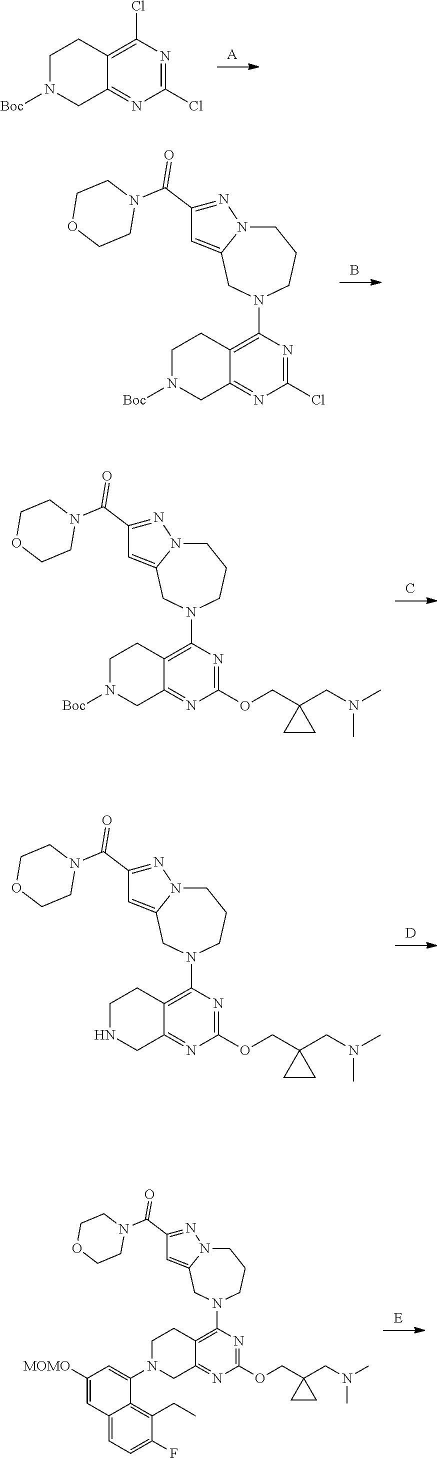

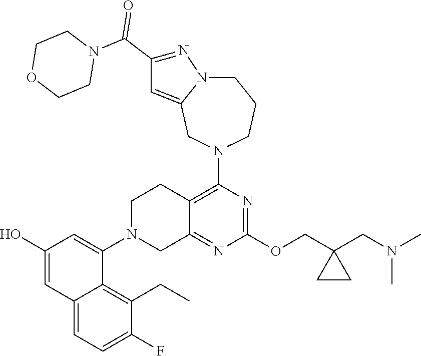 [SMILES: CCc1c(F)ccc2cc(O)cc(N3CCc4c(nc(OCC5(CN(C)C)CC5)nc4N4CCCn5nc(C(=O)N6CCOCC6)cc5C4)C3)c12]

Step A. tert-butyl 2-chloro-4-(2-(morpholine-4-carbonyl)-7,8-dihydro-4H-pyrazolo[1,5-a][1,4]diazepin-5(6H)-yl)-5,6-dihydropyrido[3,4-d]pyrimidine-7(8H)-carboxylate: To a mixture of tert-butyl 2,4-dichloro-5,6-dihydropyrido[3,4-d]pyrimidine-7(8H)-carboxylate (0.05 g, 1.0 equiv) and morpholino(5,6,7,8-tetrahydro-4H-pyrazolo[1,5-a][1,4]diazepin-2-yl)methanone (49.4 mg, 1.2 equiv) in DMF (1.0 mL) were added N,N-diethylpropan-2-amine (127 mg, 6.0 equiv) and 4A molecular sieve (50 mg, 1.0 equiv) under nitrogen. The mixture was stirred at 40° C. for 6 hours. The mixture was filtered and purified by reversed phase flash chromatography [C18, 0.1% formic acid condition] to afford the title compound (70.0 mg, 82% yield) as white solid. LCMS (ESI, M-55): m/z=462.0

Step B. tert-butyl 2-((1-((dimethylamino)methyl)cyclopropyl)methoxy)-4-(2-(morpholine-4-carbonyl)-7,8-dihydro-4H-pyrazolo[1,5-a][1,4]diazepin-5(6H)-yl)-5,6-dihydropyrido[3,4-d]pyrimidine-7(8H)-carboxylate: To a mixture of tert-butyl 2-chloro-4-(2-(morpholine-4-carbonyl)-7,8-dihydro-4H-pyrazolo[1,5-a][1,4]diazepin-5(6H)-yl)-5,6-dihydropyrido[3,4-d]pyrimidine-7(8H)-carboxylate (0.5 g, 1.0 equiv) and (1-((dimethylamino)methyl)cyclopropyl)methanol (150 mg, 1.2 equiv) in toluene (5.0 mL) were added Cs₂CO₃ (943 mg, 3 equiv), BINAP (120 mg, 0.2 equiv), and Pd(OAc)₂ (21.7 mg, 0.1 equiv). The reaction was degassed and purged with nitrogen 3 times. The reaction was heated to 110° C. and stirred for 2 hours. The reaction was diluted with water (10 mL) and extracted with ethyl acetate (3×5 mL). The combined organic layers were washed with brine (10 mL), dried over anhydrous sodium sulfate, filtered, and purified by reversed phase flash chromatography [C18, 0.1% formic acid condition] to afford the title compound (315 mg, 51% yield) as white solid; 1H NMR (400 MHz, dimethylsulfoxide-d₆) δ=6.56 (s, 1H), 4.73 (s, 2H), 4.48-4.39 (m, 2H), 4.26 (br s, 2H), 4.02-4.00 (m, 2H), 3.99-3.81 (m, 4H), 3.58 (br d, J=0.8 Hz, 6H), 3.45 (br s, 2H), 2.68-2.63 (m, 2H), 2.17 (s, 2H), 2.13 (s, 6H), 2.03 (br s, 2H), 1.44 (s, 9H), 0.58-0.52 (m, 2H), 0.38-0.31 (m, 2H); LCMS (ESI, M+1): m/z=611.5.

Step C. (5-(2-((1-((dimethylamino)methyl)cyclopropyl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(morpholino)methanone: To a solution of tert-butyl 2-((1-((dimethylamino)methyl)cyclopropyl)methoxy)-4-(2-(morpholine-4-carbonyl)-7,8-dihydro-4H-pyrazolo[1,5-a][1,4]diazepin-5(6H)-yl)-5,6-dihydropyrido[3,4-d]pyrimidine-7(8H)-carboxylate (0.31 g, 1 equiv) in DCM (3.0 mL) was added TFA (4.62 g, 79 equiv) at 0° C. under nitrogen. The mixture was stirred at 20° C. for 2 hours. The mixture was concentrated and adjusted to pH=10 with saturated Na₂CO₃ aqueous solution and 15% NaOH aqueous solution, extracted with a mixture solution (dichloromethane/methanol 10/1, 8 mL×3), dried over anhydrous sodium sulfate, filtered, and purified by reversed phase flash chromatography [C18, 0.1% formic acid condition] to afford the title compound (170 mg, 63% yield) as white solid; LCMS (ESI, M+1): m/z=511.4

Step D. (5-(2-((1-((dimethylamino)methyl)cyclopropyl)methoxy)-7-(8-ethyl-7-fluoro-3-(methoxymethoxy)naphthalen-1-yl)-5,6,7,8-tetrahydropyrido[3,4-d]pyrimidin-4-yl)-5,6,7,8-tetrahydro-4H-pyrazolo[1,5-a][1,4]diazepin-2-yl)(morpholino)methanone: To a mixture of (5-(2-((1-((dimethylamino)methyl)cyclopropyl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(morpholino)methanone (150 mg, 1.0 equiv) and 8-ethyl-7-fluoro-3-(methoxymethoxy)naphthalen-1-yl trifluoromethanesulfonate (146 mg, 1.3 equiv) in toluene (1.5 mL) were added Pd₂(dba)₃ (26.9 mg, 0.1 equiv), Cs₂CO3 (287 mg, 3.0 equiv), and Xantphos (34.0 mg, 0.2 equiv) under nitrogen. The reaction was stirred at 110° C. for 8 hours. The reaction was diluted with water (20 mL) and extracted with ethyl acetate (3×30 mL). The combined organic layers were washed with brine (30 mL), dried over anhydrous sodium sulfate, filtered, and purified by reversed phase flash chromatography [C18, 0.1% formic acid condition] to afford the title compound (150 mg, 66% yield) as yellow solid; LCMS (ESI, M+1): m/z=743.7

Step E. (5-(2-((1-((dimethylamino)methyl)cyclopropyl)methoxy)-7-(8-ethyl-7-fluoro-3-hydroxynaphthalen-1-yl)-5,6,7,8-tetrahydropyrido[3,4-d]pyrimidin-4-yl)-5,6,7,8-tetrahydro-4H-pyrazolo[1,5-al[1,4]diazepin-2-yl)(morpholino)methanone: To a solution of (5-(2-((1-((dimethylamino)methyl)cyclopropyl)methoxy)-7-(8-ethyl-7-fluoro-3-(methoxymethoxy)naphthalen-1-yl)-5,6,7,8-tetrahydropyrido[3,4-d]pyrimidin-4-yl)-5,6,7,8-tetrahydro-4H-pyrazolo[1,5-a][1,4]diazepin-2-yl)(morpholino)methanone (130 mg, 1.0 equiv) in ACN (1.5 mL) was added HCl•MeOH (4 M, 1.5 mL, 34 equiv) at 0° C. under nitrogen. The reaction was stirred at 20° C. for 1 hour. The reaction was concentrated and purified by prep-HPLC [Waters Xbridge C18 150×50 mm×10 μm; A: water (NH4HCO3), B: ACN]; B %: 31%-61% over 8 min] to afford the title compound (57.6 mg, 47% yield) as orange solid; ¹H NMR (400 MHz, dimethylsulfoxide-d₆) δ=9.65 (s, 1H), 7.66-7.52 (m, 1H), 7.24 (t, J=9.6 Hz, 1H), 6.99 (s, 2H), 6.55 (s, 1H), 5.06-4.94 (m, 1H), 4.74 (br d, J=16.8 Hz, 1H), 4.50 (br d, J=4.8 Hz, 2H), 4.18-4.07 (m, 1H), 4.06-3.97 (m, 3H), 3.92-3.75 (m, 3H), 3.65-3.54 (m, 7H), 3.49-3.36 (m, 2H), 3.27 (br s, 2H), 3.21-3.03 (m, 4H), 2.18-2.11 (m, 8H), 1.07 (t, J=7.2 Hz, 3H), 0.58-0.52 (m, 2H), 0.39-0.31 (m, 2H); ¹⁹F NMR (400 MHz, dimethylsulfoxide-d₆) δ=−121.334; LCMS (ESI, M+1): m/z =699.9.

### Example 430

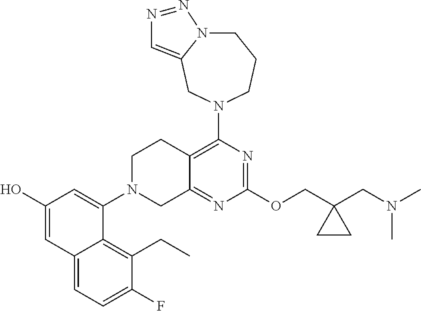 [SMILES: CCc1c(F)ccc2cc(O)cc(N3CCc4c(nc(OCC5(CN(C)C)CC5)nc4N4CCCn5nncc5C4)C3)c12]

### 4-(4-(7,8-dihydro-4H-[1,2,3]triazolo[1,5-a][1,4]diazepin-5(6H)-yl)-2-((1-((dimethylamino)methyl)cyclopropyl)methoxy)-5,6-dihydropyrido[3,4-d]pyrimidin-7(8H)-yl)-5-ethyl-6-fluoronaphthalen-2-ol

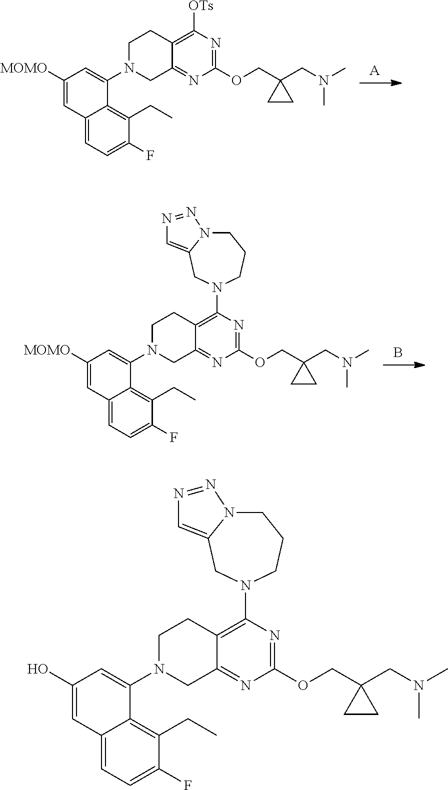

Synthesized according to Example 248. The title compound was obtained as yellow solid (FA salt). 1H NMR (400 MHz, METHANOL-d₄) δ=7.66 (s, 1H), 7.54-7.49 (m, 1H), 7.19 (s, 1H), 6.99-6.95 (m, 2H), 5.11-5.04 (m, 1H), 4.95-4.90 (m, 1H), 4.75-4.69 (m, 2H), 4.22-4.03 (m, 5H), 3.70-3.64 (m, 1H), 3.57-3.51 (m, 1H), 3.43-3.34 (m, 2H), 3.26-3.13 (m, 2H), 2.77-2.61 (m, 3H), 2.56-2.38 (m, 6H), 2.35-2.26 (m, 1H), 2.13-2.03 (m, 1H), 1.11 (t, J=6.0 Hz, 3H), 0.75-0.68 (m, 2H), 0.64-0.54 (m, 2H); LCMS (ESI, M+1): m/z=587.3.

### Example 431

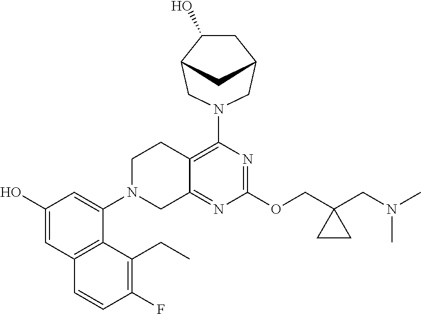 [SMILES: CCc1c(F)ccc2cc(O)cc(N3CCc4c(nc(OCC5(CN(C)C)CC5)nc4N4C[C@@H]5C[C@H](C4)[C@H](O)C5)C3)c12]

### (1R,5R,6R)-3-(2-((1-((dimethylamino)methyl)cyclopropyl)methoxy)-7-(8-ethyl-7-fluoro-3-hydroxynaphthalen-1-yl)-5,6,7,8-tetrahydropyrido[3,4-d]pyrimidin-4-yl)-3-azabicyclo[3.2.1]octan-6-ol

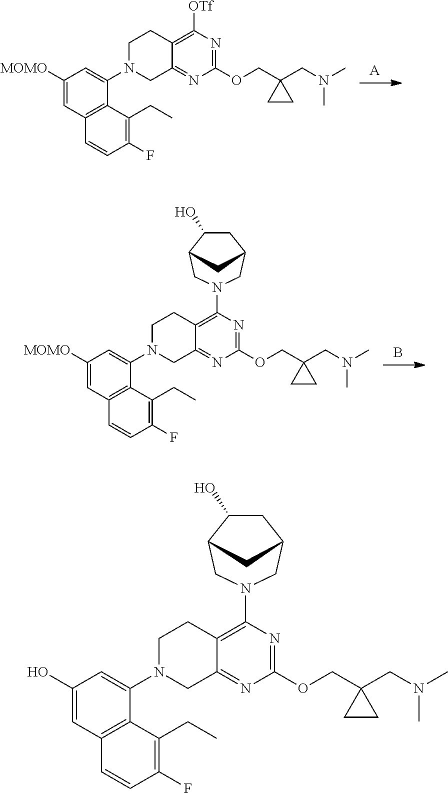

Synthesized according to Example 248. The title compound was obtained as white solid (FA salt). ¹H NMR (400 MHz, METHANOL-d₄) δ=7.54-7.47 (m, 1H), 7.17-7.10 (m, 1H), 7.03-6.90 (m, 2H), 4.67-4.45 (m, 1H), 4.41-4.13 (m, 4H), 4.11-4.01 (m, 1H), 3.91-3.52 (m, 2H), 3.48-3.34 (m, 3H), 3.26-3.10 (m, 2H), 3.09-2.91 (m, 2H), 2.90-2.73 (m, 2H), 2.72-2.59 (m, 6H), 2.36-2.12 (m, 3H), 1.82-1.65 (m, 2H), 1.62-1.21 (m, 1H), 1.34-1.10 (m, 3H), 0.85-0.72 (m, 2H), 0.66 (br d, J=4.8 Hz, 2H); LCMS (ESI, M+1): m/z=576.5.

### Example 432

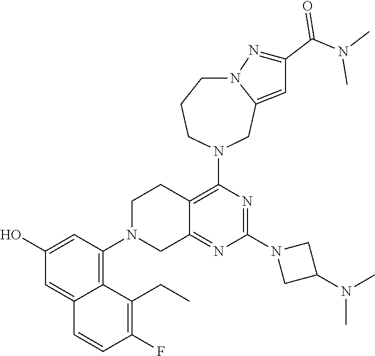 [SMILES: CCc1c(F)ccc2cc(O)cc(N3CCc4c(nc(N5CC(N(C)C)C5)nc4N4CCCn5nc(C(=O)N(C)C)cc5C4)C3)c12]

### 5-(2-(3-(dimethylamino)azetidin-1-yl)-7-(8-ethyl-7-fluoro-3-hydroxynaphthalen-1-yl)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide

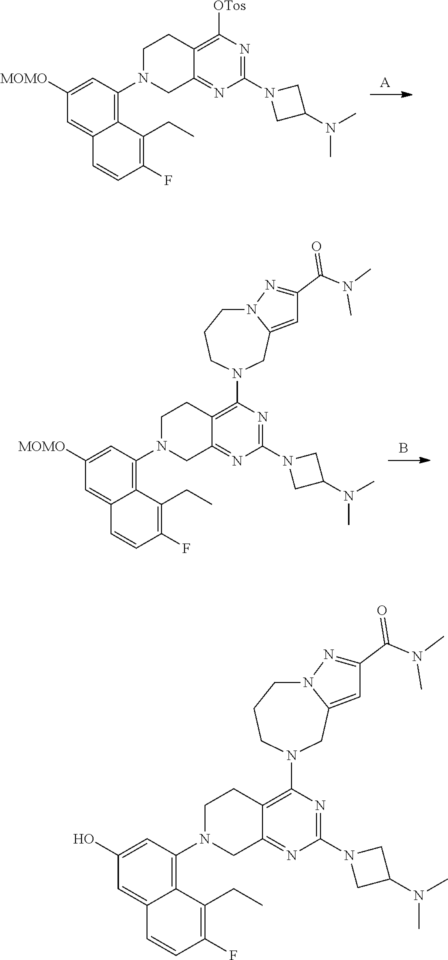

Synthesized according to Example 248. The title compound was obtained as yellow solid (FA salt). ¹H NMR (400 MHz, METHANOL-d₄) δ=7.50 (dd, J=5.6, 8.8 Hz, 1H), 7.14 (t, J=9.2 Hz, 1H), 6.95 (s, 2H), 6.56 (s, 1H), 4.80-4.70 (m, 2H), 4.57-4.46 (m, 2H), 4.19-4.01 (m, 4H), 3.99-3.84 (m, 3H), 3.60 (br d, J=17.2 Hz, 1H), 3.52-3.45 (m, 1H), 3.44-3.35 (m, 3H), 3.35-3.32 (m, 3H), 3.21-3.11 (m, 2H), 3.07 (s, 3H), 2.68-2.58 (m, 1H), 2.39-2.22 (m, 7H), 2.05-1.94 (m, 1H), 1.11 (t, J=7.2 Hz, 3H) LCMS (ESI, M+1): m/z=628.3.

### Example 433

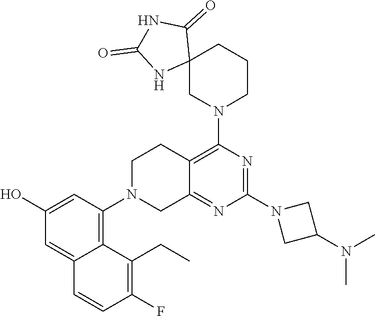 [SMILES: CCc1c(F)ccc2cc(O)cc(N3CCc4c(nc(N5CC(N(C)C)C5)nc4N4CCCC5(C4)NC(=O)NC5=O)C3)c12]

### 7-(2-(3-(dimethylamino)azetidin-1-yl)-7-(8-ethyl-7-fluoro-3-hydroxynaphthalen-1-yl)-5,6,7,8-tetrahydropyrido[3,4-d]pyrimidin-4-yl)-1,3,7-triazaspiro[4.5]decane-2,4-dione

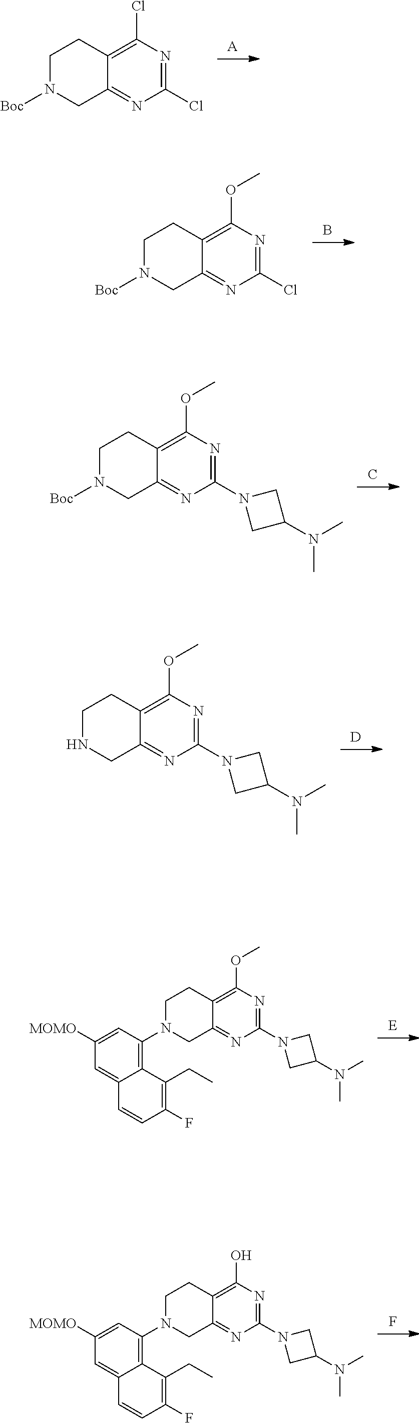

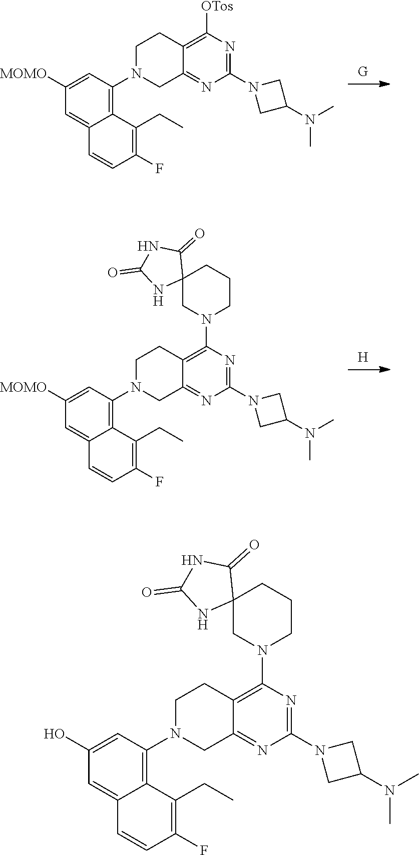

Step A. tert-butyl 2-chloro-4-methoxy-5,6-dihydropyrido[3,4-d]pyrimidine-7(8H)-carboxylate: To a solution of tert-butyl 2,4-dichloro-5,6-dihydropyrido[3,4-d]pyrimidine-7(8H)-carboxylate (15.0 g, 1.0 equiv) in MeOH (100 mL) was added a solution of NaOMe (5.33 g, 2.0 equiv) in MeOH (50 mL) dropwise at 0° C. The reaction was stirred at 20° C. for 1 hour. The mixture was quenched with water (100 mL), concentrated under vacuum to remove MeOH, and extracted with ethyl acetate (2×200 mL). The combined organic layers were dried over anhydrous sodium sulfate, concentrated, and purified with reversed phase flash chromatography [water (FA, 0.1%)/acetonitrile] to afford the title compound (14.0 g, 87% yield) as white solid.

Step B. tert-butyl 2-(3-(dimethylamino)azetidin-1-yl)-4-methoxy-5,6-dihydropyrido[3,4-d]pyrimidine-7(8H)-carboxylate: To a mixture of N,N-dimethylazetidin-3-amine (11.6 g, 2 equiv, 2HCl) and tert-butyl 2-chloro-4-methoxy-6,8-dihydro-5H-pyrido[3,4-d]pyrimidine-7-carboxylate (10.0 g, 1.0 equiv) in toluene (100 mL) were added Pd(OAc)₂ (374 mg, 0.05 equiv), BINAP (4.15 g, 0.20 equiv), and Cs₂CO₃ (54.3 g, 5.00 equiv). The mixture was degassed and purged with N₂ 3 times. The reaction was stirred at 100° C. for 12 hours under N₂. The mixture was diluted with ethyl acetate (300 mL) and washed with water (300 mL) and brine (300 mL). The organic phase was dried over anhydrous sodium sulfate, concentrated, and purified with reversed phase flash chromatography [water (FA, 0.1%)/acetonitrile] to afford the title compound (11.0 g, 78% yield) as white solid, LCMS (ESI, M+1): m/z=364.2.

Step C. 1-(4-methoxy-5,6,7,8-tetrahydropyrido[3,4-d]pyrimidin-2-yl)-N,N-dimethylazetidin-3-amine: A mixture of tert-butyl 2-[3-(dimethylamino)azetidin-1-yl]-4-methoxy-6,8-dihydro-5H-pyrido[3,4-d]pyrimidine-7-carboxylate (10.0 g, 1.0 equiv) in HCl-MeOH (4 M, 14 equiv) was stirred at 25° C. for 0.5 hours. The reaction was concentrated under reduced pressure to remove HCl MeOH and dissolved in water (100 mL). The pH value of the mixture was adjusted to 11 with NaOH solution and the mixture was extracted with ethyl acetate (2×100 mL). The combined organic layers were dried over anhydrous sodium sulfate and concentrated to afford the title compound (5.5 g, 53%) as yellow solid.

Step D. 1-[7-[8-ethyl-7-fluoro-3-(methoxymethoxy)-1-naphthyl]-4-methoxy-6,8-dihydro-5H-pyrido[3,4-d]pyrimidin-2-yl]-N,N-dimethyl-azetidin-3-amine: To a mixture of 1-(4-methoxy-5,6,7,8-tetrahydropyrido[3,4-d]pyrimidin-2-yl)-N,N-dimethyl-azetidin-3-amine (3.00 g, 1.0 equiv) and [8-ethyl-7-fluoro-3-(methoxymethoxy)-1-naphthyl]trifluoromethanesulfonate (8.71 g, 2.0 equiv) in toluene (30 mL) were added Pd₂(dba)₃ (1.04 g, 0.1 equiv), Xantphos (1.32 g, 0.2 equiv), and Cs₂CO₃ (11.1 g, 3.0 equiv). The mixture was degassed and purged with N₂ 3 times. The reaction was stirred at 100° C. for 36 hours under N₂ atmosphere. The mixture was diluted with ethyl acetate (200 mL) and washed with water (100 mL) and brine (100 mL). The organic layer was dried over anhydrous sodium sulfate, concentrated, and purified with column chromatography (Al₂O₃, Petroleum ether/Ethyl acetate=50/1 to 1/1) to afford the title compound (9.00 g, 63.8% yield) as brown solid. LCMS (ESI, M+1): m/z=496.1.

Step E. 2-(3-(dimethylamino)azetidin-1-yl)-7-(8-ethyl-7-fluoro-3-(methoxymethoxy)naphthalen-1-yl)-5,6,7,8-tetrahydropyrido[3,4-d]pyrimidin-4-ol: To a solution of EtSH (1.48 g, 5.9 equiv) in DMAC (10 mL) was added NaH (484 mg, 60% purity, 3.0 equiv). The mixture was stirred at 0° C. for 0.5 hours. A solution of 1-[7-[8-ethyl-7-fluoro-3-(methoxymethoxy)-1-naphthyl]-4-methoxy-6,8-dihydro-5H-pyrido[3,4-d]pyrimidin-2-yl]-N,N-dimethyl-azetidin-3-amine (2.00 g, 1.0 equiv) in DMAC (8 mL) was added into the mixture. The reaction was stirred at 60° C. for 0.5 hours. The mixture was quenched with water (50 ml). The pH value of the mixture was adjusted to 5 with 2 N HCl. The mixture was extracted with ethyl acetate (100 mL×3) and washed with brine (100 mL). The combined organic layers were dried over anhydrous sodium sulfate and concentrated to afford the title compound (2.5 g, crude) as yellow solid. LCMS (ESI, M+1): m/z=482.3

Step F 2-(3-(dimethylamino)azetidin-1-yl)-7-(8-ethyl-7-fluoro-3-(methoxymethoxy)naphthalen-1-yl)-5,6,7,8-tetrahydropyrido[3,4-d]pyrimidin-4-yl 4-methylbenzenesulfonate: To a mixture of 4-methylbenzenesulfonyl chloride (594 mg, 1 equiv), DMAP (38.1 mg, 0.10 equiv), and 2-[3-(dimethylamino)azetidin-1-yl]-7-[8-ethyl-7-fluoro-3-(methoxymethoxy)-1-naphthyl]-6,8-dihydro-5H-pyrido[3,4-d]pyrimidin-4-ol (1.50 g, 1.0 equiv) in DCM (15 mL) was added N,N-diethylpropan-2-amine (1.21 g, 3.0 equiv). The reaction was stirred at 25° C. for 1 hour. The mixture was diluted with DCM (50 mL) and washed with water (50 mL) and brine (50 mL). The organic layer was dried over anhydrous sodium sulfate, concentrated, and purified with reversed phase flash chromatography [water (FA, 0.1%)/acetonitrile] to afford the title compound (330 mg, 16% yield) as white solid, LCMS (ESI, M+1): m/z=636.4.

The last two steps were performed according to Example 248. The title compound was obtained as orange solid (FA salt). ¹H NMR (400 MHz, METHANOL-d₄) δ=7.51 (dd, J=5.2, 8.4 Hz, 1H), 7.14 (t, J=9.2 Hz, 1H), 7.05-6.87 (m, 2H), 4.14 (br s, 2H), 4.01 (br s, 5H), 3.60 (br dd, J=2.8, 17.2 Hz, 1H), 3.53-3.38 (m, 3H), 3.34 (br d, J=7.2 Hz, 1H), 3.29-3.24 (m, 1H), 3.20-3.03 (m, 2H), 2.96-2.64 (m, 2H), 2.41 (d, J=3.6 Hz, 6H), 2.13-2.03 (m, 1H), 2.01-1.77 (m, 3H), 1.10 (dt, J=3.2, 7.2 Hz, 3H); LCMS (ESI, M+1): m/z=589.2.

### Example 434

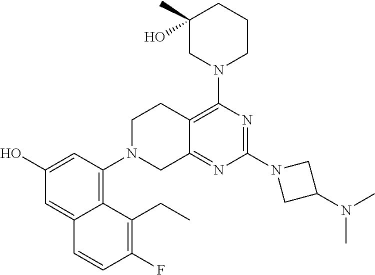 [SMILES: CCc1c(F)ccc2cc(O)cc(N3CCc4c(nc(N5CC(N(C)C)C5)nc4N4CCC[C@@](C)(O)C4)C3)c12]

### (R)-1-(2-(3-(dimethylamino) azetidin-1-yl)-7-(8-ethyl-7-fluoro-3-hydroxynaphthalen-1-yl) -5,6,7,8-tetrahydropyrido [3,4-d]pyrimidin-4-yl)-3-methylpiperidin-3-ol

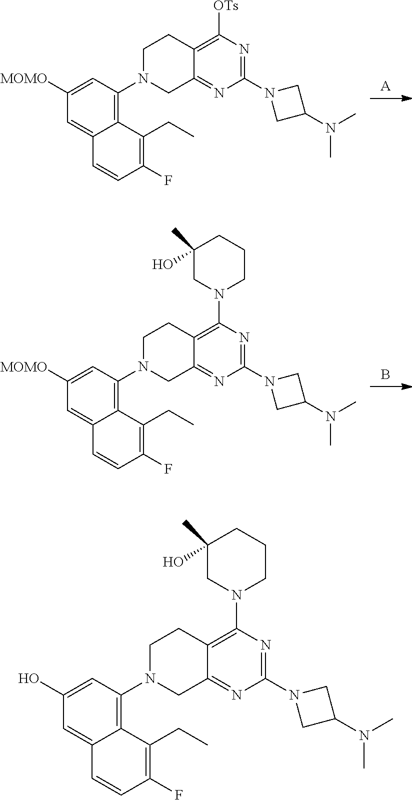

Synthesized according to Example 248. The title compound was obtained as white solid (FA salt). ¹H NMR (400 MHz, METHANOL-d₄) δ=7.55-7.46 (m, 1H), 7.14 (t, J=9.6 Hz, 1H), 7.01-6.93 (m, 1H), 4.21-4.01 (m, 3H), 3.96-3.86 (m, 2H), 3.79-3.39 (m, 6H), 3.38-3.32 (m, 2H), 3.26-2.98 (m, 3H), 2.65 (br dd, J=4.4, 11.2 Hz, 1H), 2.33 (d, J=4.8 Hz, 6H), 2.05-1.58 (m, 4H), 1.30-1.18 (m, 3H), 1.11 (t, J=7.2 Hz, 3H); LCMS (ESI, M+1): m/z=535.4.

### Example 435

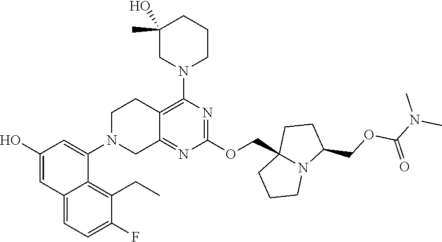 [SMILES: CCc1c(F)ccc2cc(O)cc(N3CCc4c(nc(OC[C@]56CCCN5[C@H](COC(=O)N(C)C)CC6)nc4N4CCC[C@@](C)(O)C4)C3)c12]

### ((3S,7aR)-7a-(((7-(8-ethyl-7-fluoro-3-hydroxynaphthalen-1-yl)-4-((R)-3-hydroxy-3-methylpiperidin-1-yl)-5,6,7,8-tetrahydropyrido[3,4-d]pyrimidin-2-yl)oxy)methyl)hexahydro-1H-pyrrolizin-3-yl)methyl dimethylcarbamate

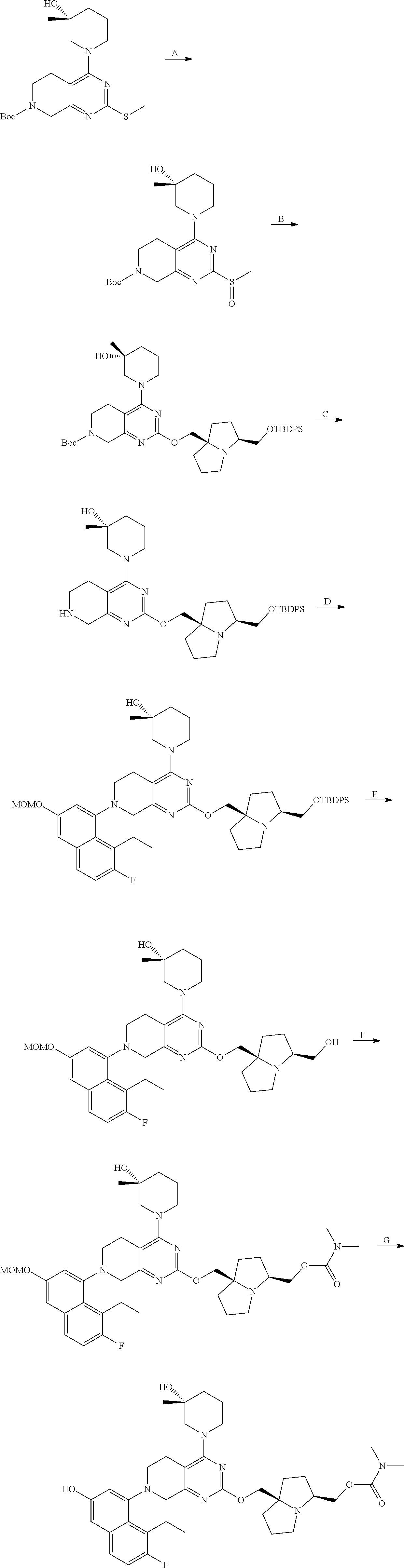

Step A. tert-butyl 4-((R)-3-hydroxy-3-methylpiperidin-1-yl)-2-(methylsulfinyl)-5,6-dihydropyrido[3,4-d] pyrimidine-7(8H)-carboxylate: To a solution of (R)-tert-butyl 4-(3-hydroxy-3-methylpiperidin-1-yl)-2-(methylthio)-5,6-dihydropyrido[3,4-d]pyrimidine-7(8H)-carboxylate (1.80 g, 1.0 equiv) in DCM (20 mL) was added m-CPBA (926 mg, 85% purity, 1.0 equiv). The reaction was stirred at 20° C. for 0.5 hours. To the mixture was added saturated NaHCO₃ aqueous solution (2×15 mL). The combined organic layers were washed brine (2×15 mL), dried over anhydrous sodium sulfate, and concentrated under reduced pressure to afford the title compound (1.70 g, 91% yield) as yellow solid; LCMS (ESI, M+1): m/z=411.2.

Step B. tert-butyl 2-(((3 S,7aR)-3-(((tert-butyldiphenvlsilyl)oxy)methyl)hexahydro-1H-pyrrolizin-7a-yl)methoxy)-4-((R)-3-hydroxy-3-methylpiperidin-1-yl)-5,6-dihydropyrido[3,4-d] pyrimidine-7(8H)-carboxylate: To a mixture of tert-butyl 4-((R)-3-hydroxy-3-methylpiperidin-1-yl)-2-(methylsulfinyl)-5,6-dihydropyrido[3,4-d]pyrimidine-7(8H)-carboxylate (1.70 g, 1.0 equiv), ((3S,7aS)-3-(((tert-butyldiphenylsilyl)oxy)methyl)tetrahydro-1H-pyrrolizin-7a(5H)-yl)methanol (1.36 g, 0.80 equiv), and 4A molecular sieve (200 mg) in toluene (20 mL) was added sodium;2-methylpropan-2-olate (1.19 g, 3.0 equiv). The mixture was stirred at 0° C. for 0.5 hours. The reaction was filtered to give a solution. The mixture was extracted with EtOAc (3×30 mL). The combined organic layers were dried over anhydrous sodium sulfate, concentrated, and purified by reversed-phase HPLC (0.1% FA condition) to afford the title compound (1.60 g, 51% yield) as yellow solid; 1H NMR (400 MHz, CHLOROFORM-d) δ=7.75-7.68 (m, 4H), 7.43-7.34 (m, 6H), 4.55 (br d, J=18.4 Hz, 1H), 4.38-4.32 (m, 1H), 4.20-4.05 (m, 2H), 4.00-3.94 (m, 1H), 3.93-3.88 (m, 1H), 3.72-3.61 (m, 2H), 3.57-3.48 (m, 2H), 3.36-3.26 (m, 1H), 3.02-2.93 (m, 2H), 2.92-2.89 (m, 1H), 2.88-2.76 (m, 2H), 2.69-2.56 (m, 3H), 2.22-2.13 (m, 1H), 2.05 (s, 2H), 2.04-1.98 (m, 1H), 1.94-1.87 (m, 1H), 1.83-1.78 (m, 3H), 1.71-1.63 (m, 3H), 1.50-1.47 (m, 9H), 1.27 (t, J=7.2 Hz, 3H), 1.06 (s, 9H); LCMS (ESI, M+1): m/z=757.1.

Step C. (R)-1-(2-(((3S,7aR)-3-(((tert-butyldiphenvlsilyl)oxy)methyl)hexahydro-1H-pyrrolizin-7a-yl)methoxy)-5,6,7,8-tetrahydropyrido[3,4-d]pyrimidin-4-yl)-3-methylpiperidin-3-ol: A solution of tert-butyl 2-(((3S,7aR)-3-(((tert-butyldiphenylsilyl)oxy)methyl)hexahydro-1H-pyrrolizin-7a-yl)methoxy)-4-((R)-3-hydroxy-3-methylpiperidin-1-yl)-5,6-dihydropyrido[3,4-d]pyrimidine-7(8H)-carboxylate (1.00 g, 1.0 equiv) in DCM (5 mL) and TFA (5 mL) was stirred at 25° C. for 0.5 hours. The reaction was concentrated under reduced pressure to remove solvent. To the residue was added saturated NaHCO₃ aqueous solution to adjust the pH˜7 and extracted with EtOAc (3×10 mL). The combined organic layers were dried over anhydrous sodium sulfate, concentrated, and purified by reversed phase HPLC (0.1% FA condition) to afford the title compound (634 mg, 73% yield) as yellow solid.

Step D. (R)-1-(2-(((3S,7aR)-3-(((tert-butyldiphenvlsilyl)oxy)methyl)hexahydro-1H-pyrrolizin-7a-yl)methoxy)-7-(8-ethyl-7-fluoro-3-(methoxymethoxy)naphthalen-1-yl)-5,6,7,8-tetrahydropyrido[3,4-d]pyrimidin-4-yl)-3-methylpiperidin-3-ol: To a solution of (R)-1-(2-(((3S,7aR)-3-(((tert-butyldiphenylsilyl)oxy)methyl)hexahydro-1H-pyrrolizin-7a-yl)methoxy)-5,6,7,8-tetrahydropyrido[3,4-d]pyrimidin-4-yl)-3-methylpiperidin-3-ol (500 mg, 1.0 equiv), 8-ethyl-7-fluoro-3-(methoxymethoxy)naphthalen-1-yl trifluoromethanesulfonate (729 mg, 2.5 equiv), and Cs₂CO3 (745 mg, 3.0 equiv) in toluene (10 mL) was added methanesulfonato[(4,5-bis(diphenylphosphino)-9,9-dimethylxanthene)-2-(2V′-amino-1,1-biphenyl)]palladium(ii) (72.0 mg, 0.1 equiv) and Xantphos (44.0 mg, 0.1 equiv). The mixture was degassed and purged with nitrogen 3 times. The reaction was stirred at 100° C. for 24 hours under nitrogen. The mixture was diluted with H₂O (20 mL) and extracted with EtOAc (3×15 mL). The combined organic layers were dried over anhydrous sodium sulfate, concentrated, and purified by column chromatography (SiO2, EtOAc/MeOH=30/1 to 1/1) and purified by prep-HPLC (column: Phenomenex Luna C18 200×40 mm×10 μm; mobile phase: [water (FA)-ACN]; B %: 43%-73%, 10 min) to afford the title compound (100 mg, 15% yield) as purple solid; LCMS (ESI, M+1): m/z=888.8.

Step E. (R)-1-(7-(8-ethyl-7-fluoro-3-(methoxymethoxy)naphthalen-1-yl)-2-(((3S,7aR)-3-(hydroxymethyl)hexahydro-1H-pyrrolizin-7a-yl)methoxy)-5,6,7,8-tetrahydropyrido[3,4-d]pyrimidin-4-yl)-3-methylpiperidin-3-ol: To a solution of (R)-1-(2-(((3S,7aR)-3-(((tert-butyldiphenylsilyl)oxy)methyl)hexahydro-1H-pyrrolizin-7a-yl)methoxy)-7-(8-ethyl-7-fluoro-3-(methoxymethoxy)naphthalen-1-yl)-5,6,7,8-tetrahydropyrido[3,4-d]pyrimidin-4-yl)-3-methylpiperidin-3-ol (170 mg, 1.0 equiv) in DMF (2 mL) was added CsF (291 mg, 10 equiv). The reaction was stirred at 40° C. for 2 hours. The reaction was filtered and purified by reversed phase HPLC (0.1% FA condition) to afford the title compound (120 mg, 96% yield) as yellow solid; LCMS (ESI, M+1): m/z=650.7.

Step F. ((3S,7aR)-7a-(((7-(8-ethyl-7-fluoro-3-(methoxymethoxy)naphthalen-1-yl)-4-((R)-3-hydroxy-3-methylpiperidin-1-yl)-5,6,7,8-tetrahydropyrido[3,4-d]pyrimidin-2-yl)oxy)methyl)hexahydro-1H-pyrrolizin-3-yl)methyl dimethylcarbamate: To a solution of (R)-1-(7-(8-ethyl-7-fluoro-3-(methoxymethoxy)naphthalen-1-yl)-2-(((3S,7aR)-3-(hydroxymethyl)hexahydro-1H-pyrrolizin-7a-yl)methoxy)-5,6,7,8-tetrahydropyrido[3,4-d]pyrimidin-4-yl)-3-methylpiperidin-3-ol (170 mg, 1.0 equiv) in THE (2 mL) was added NaH (157 mg, 60% purity, 15 equiv). After the reaction was stirred 0° C. for 0.5 hours, dimethylcarbamic chloride (56.0 mg, 2.0 equiv) was added into the mixture. The reaction was stirred at 0° C. for 0.5 hours. The reaction was quenched with H₂O (5 mL) at 0° C. and extracted with EtOAc (3×2 mL). The combined organic layers were dried over anhydrous sodium sulfate and concentrated under reduced pressure to afford the title compound (200 mg, crude) as yellow solid; LCMS (ESI, M+1): m/z=721.4.

Step G. ((3S,7aR)-7a-(((7-(8-ethyl-7-fluoro-3-hydroxynaphthalen-1-yl)-4-((R)-3-hydroxy-3-methylpiperidin-1-yl)-5,6,7,8-tetrahydropyrido[3,4-d]pyrimidin-2-yl)oxy)methyl)hexahydro-1H-pyrrolizin-3-yl)methyl dimethylcarbamate: A solution of ((3S,7aR)-7a-(((7-(8-ethyl-7-fluoro-3-(methoxymethoxy)naphthalen-1-yl)-4-((R)-3-hydroxy-3-methylpiperidin-1-yl)-5,6,7,8-tetrahydropyrido[3,4-d]pyrimidin-2-yl)oxy)methyl)hexahydro-1H-pyrrolizin-3-yl)methyl dimethylcarbamate (100 mg, 1.0 equiv) in HCl-MeOH (4 M, 1 mL, 29 equiv) was stirred at 25° C. for 0.5 hours. The mixture was concentrated under reduced pressure to remove solvent. To the residue was added saturated NaHCO₃ aqueous solution to adjust the pH-7 and extracted with EtOAc (3×2 mL). The combined organic layers were dried over anhydrous sodium sulfate, concentrated, and purified by prep-HPLC (column: Welch Ultimate C18 150×25 mm×5 μm; mobile phase: [water (TFA)-ACN]; B %: 23%-53%, 10 min) to afford the title compound (18 mg) as yellow solid (TFA salt). ¹H NMR (400 MHz, METHANOL-d₄) δ=7.53 (dd, J=6.0, 9.2 Hz, 1H), 7.17 (t, J=9.2 Hz, 1H), 7.04-6.99 (m, 2H), 4.67-4.54 (m, 2H), 4.53-4.43 (m, 1H), 4.35 (dd, J=7.6, 12.8 Hz, 1H), 4.21-4.00 (m, 2H), 3.97-3.78 (m, 2H), 3.77-3.61 (m, 2H), 3.58-3.49 (m, 2H), 3.46-3.32 (m, 3H), 3.26-3.13 (m, 2H), 2.92 (s, 3H), 2.89-2.83 (m, 3H), 2.74 (br d, J=14.4 Hz, 1H), 2.49-2.37 (m, 1H), 2.34-2.06 (m, 8H), 1.97-1.86 (m, 1H), 1.98-1.66 (m, 3H), 1.26 (d, J=18.0 Hz, 3H), 1.13 (dt, J=4.0, 7.2 Hz, 3H); LCMS (ESI, M+1): m/z=677.6.

### Example 436

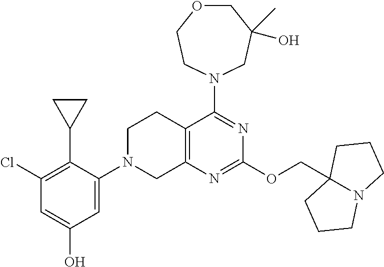 [SMILES: CC1(O)COCCN(c2nc(OCC34CCCN3CCC4)nc3c2CCN(c2cc(O)cc(Cl)c2C2CC2)C3)C1]

### 4-(7-(3-chloro-2-cyclopropyl-5-hydroxyphenyl)-2-((tetrahydro-1H-pyrrolizin-7a(5H)-yl)methoxy)-5,6,7,8-tetrahydropyrido[3,4-d]pyrimidin-4-yl)-6-methyl-1,4-oxazepan-6-ol

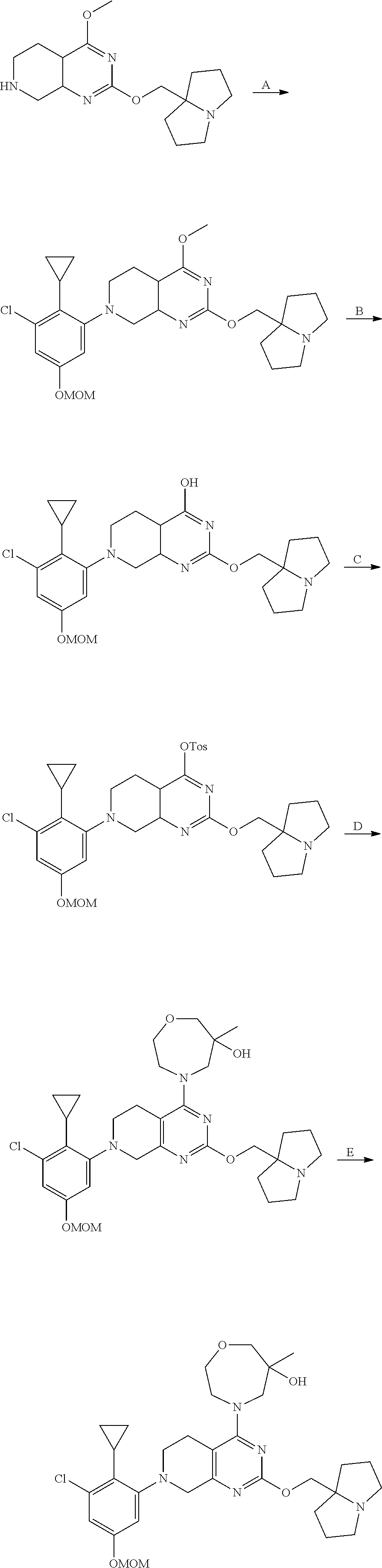

Step A: 7-(3-chloro-2-cyclopropyl-5-(methoxymethoxy)phenyl)-4-methoxy-2-((tetrahydro-1H-pyrrolizin-7a(5H)-yl)methoxy)-5,6,7,8-tetrahydropyrido[3,4-d]pyrimidine: A mixture of 4-methoxy-2-((tetrahydro-1H-pyrrolizin-7a(5H)-yl)methoxy)-5,6,7,8-tetrahydropyrido[3,4-d]pyrimidine (1.50 g, 1.0 equiv), 1-bromo-3-chloro-2-cyclopropyl-5-(methoxymethoxy)benzene (1.44 g, 1.0 equiv), Pd₂(dba)₃ (451 mg, 0.10 equiv), Xantphos (570 mg, 0.20 equiv), and cesium carbonate (4.82 g, 3.00 equiv) in toluene (15.0 mL) was degassed and purged with nitrogen three times. Then the mixture was stirred at 110° C. for 12 hours under nitrogen atmosphere. The reaction was diluted with water (200 mL) and extracted with ethyl acetate (200 mL×2). The combined organic layers were dried, filtered, concentrated, and purified withprep-HPLC [FA condition; column: Phenomenex luna C18 (250×70 mm, 10 μm); mobile phase: [water (FA)-ACN]; B %: 45%-50%, 10 minutes] to afford the title compound (1.70 g, 67% yield) as a yellow oil. ¹H NMR (400 MHz, CD3OD) δ=8.08 (s, 1H), 6.80 (d, J=2.4 Hz, 1H), 6.63 (d, J=2.4 Hz, 1H), 5.14 (s, 2H), 4.55 (s, 2H), 4.06 (s, 3H), 3.74-3.62 (m, 2H), 3.44 (s, 3H), 3.39 (br t, J=5.2 Hz, 2H), 3.36-3.32 (m, 2H), 3.30-3.26 (m, 2H), 2.77 (t, J=5.6 Hz, 2H), 2.35-2.08 (m, 8H), 1.69 (tt, J=5.6, 8.4 Hz, 1H), 1.05-0.92 (m, 2H), 0.69-0.59 (m, 2H).

Step B: 7-(3-chloro-2-cyclopropyl-5-(methoxymethoxy)phenyl)-2-((tetrahydro-1H-pyrrolizin-7a(5H)-yl)methoxy)-5,6,7,8-tetrahydropyrido[3,4-d]pyrimidin-4-ol: To a solution of ethanethiol (800 mg, 3.9 equiv) in N,N-dimethylacetamide (20.0 mL) was added sodium hydride (264 mg, 60% purity, 2.0 equiv) at 10° C. The mixture was stirred at 10° C. for 0.5 hours. Then 7-(3-chloro-2-cyclopropyl-5-(methoxymethoxy)phenyl)-4-methoxy-2-((tetrahydro-1H-pyrrolizin-7a(5H)-yl)methoxy)-5,6,7,8-tetrahydropyrido[3,4-d]pyrimidine (1.70 g, 1.0 equiv) was added to the above mixture. The reaction was stirred at 60° C. for 1 hour. The combined organic layers were dried, filtered and concentrated to afford the title compound (1.70 g, crude) as a yellow oil. LCMS (ESI, M+1): m/z=501.3.

Step C: 7-(3-chloro-2-cyclopropyl-5-(methoxymethoxy)phenyl)-2-((tetrahydro-1H-pyrrolizin-7a(5H)-yl)methoxy)-5,6,7,8-tetrahydropyrido[3,4-d]pyrimidin-4-yl 4-methylbenzenesulfonate To a solution of 7-(3-chloro-2-cyclopropyl-5-(methoxymethoxy)phenyl)-2-((tetrahydro-1H-pyrrolizin-7a(5H)-yl)methoxy)-5,6,7,8-tetrahydropyrido[3,4-d]pyrimidin-4-ol (1.70 g, 1.0 equiv) in dichloromethane (10.0 mL) were added paratoluensulfonyl chloride (970 mg, 1.5 equiv), N,N-diethylpropan-2-amine (1.32 g, 3.00 equiv), and DMAP (41.5 mg, 0.10 equiv).The mixture was stirred at 25° C. for 2 hours. The combined organic layers were dried, filtered, concentrated, and purified with column chromatography [SiO2, petroleum ether/ethyl acetate=5/1 to 0/1] to afford the title compound (700 mg, 32% yield) as a yellow oil. LCMS (ESI, M+1): m/z=655.4.

Step D: 4-(7-(3-chloro-2-cyclopropyl-5-(methoxymethoxy)phenyl)-2-((tetrahydro-1H-pyrrolizin-7a(5H)-yl)methoxy)-5,6,7,8-tetrahydropyrido[3,4-d]pyrimidin-4-yl)-6-methyl-1,4-oxazepan-6-ol: To a solution of 7-(3-chloro-2-cyclopropyl-5-(methoxymethoxy)phenyl)-2-((tetrahydro-1H-pyrrolizin-7a(5H)-yl)methoxy)-5,6,7,8-tetrahydropyrido[3,4-d]pyrimidin-4-yl 4-methylbenzenesulfonate (70.0 mg, 1.0 equiv) and 6-methyl-1,4-oxazepan-6-ol (16.8 mg, 1.2 equiv) in DMF (1.50 mL) were added N,N-diethylpropan-2-amine (41.4 mg, 3.0 equiv) and 4A molecular sieve (10.7 μmol, 0.10 equiv). The mixture was stirred at 40° C. for 16 hours. The reaction was filtered and concentrated to afford the title compound (50.0 mg, crude) as yellow solid; LCMS (ESI, M+1): m/z=614.4

Step E: 4-(7-(3-chloro-2-cyclopropyl-5-hydroxyphenyl)-2-((tetrahydro-1H-pyrrolizin-7a(5H)-yl)methoxy)-5,6,7,8-tetrahydropyrido[3,4-d]pyrimidin-4-yl)-6-methyl-1,4-oxazepan-6-ol: To a solution of 4-(7-(3-chloro-2-cyclopropyl-5-(methoxymethoxy)phenyl)-2-((tetrahydro-1H-pyrrolizin-7a(5H)-yl)methoxy)-5,6,7,8-tetrahydropyrido[3,4-d]pyrimidin-4-yl)-6-methyl-1,4-oxazepan-6-ol (50.0 mg, 1.0 equiv) in chloromethane (5.0 mL) was added HCl/MeOH (0.50 mL). The mixture was stirred at 0° C. for 1 hour. The reaction was filtered, concentrated, and purified withprep-HPLC [FA condition; column: Phenomenex C18 75×30 mm×3 μm; mobile phase: [water(FA)-ACN];B %: 12%-42%,7 minutes] to give a white solid and then was purified with prep-SFC [column: DAICEL CHIRALPAK IC (250 mm×30 mm, 10 μm); mobile phase: [0.1% NH₃H₂O ETOH]; B %: 60%-60%,3.4 minutes] to afford the title compound (7.33 mg, 15% yield, 97% purity) as off-white solid; ¹H NMR (400 MHz, DMSO-d₆) δ=9.92-9.40 (m, 1H), 8.27 (s, 1H), 6.49 (d, J=1.6 Hz, 1H), 6.36 (d, J=2.0 Hz, 1H), 4.02-3.62 (m, 10H), 3.39 (br d, J=4.0 Hz, 2H), 3.09 (br dd, J=5.2, 5.6 Hz, 1H), 2.97-2.91 (m, 2H), 2.78 (br s, 2H), 2.60-2.54 (m, 2H), 1.89-1.71 (m, 6H), 1.59-1.52 (m, 2H), 1.23 (br s, 1H), 1.04 (s, 3H), 1.01-0.92 (m, 2H), 0.56 (dt, J=5.2, 9.6 Hz, 2H); LCMS [M+1]⁺: 570.3

### Example 437

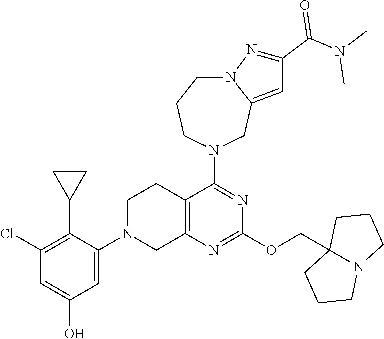 [SMILES: CN(C)C(=O)c1cc2n(n1)CCCN(c1nc(OCC34CCCN3CCC4)nc3c1CCN(c1cc(O)cc(Cl)c1C1CC1)C3)C2]

### 5-(7-(3-chloro-2-cyclopropyl-5-hydroxyphenyl)-2-((tetrahydro-1H-pyrrolizin-7a(5H)-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide

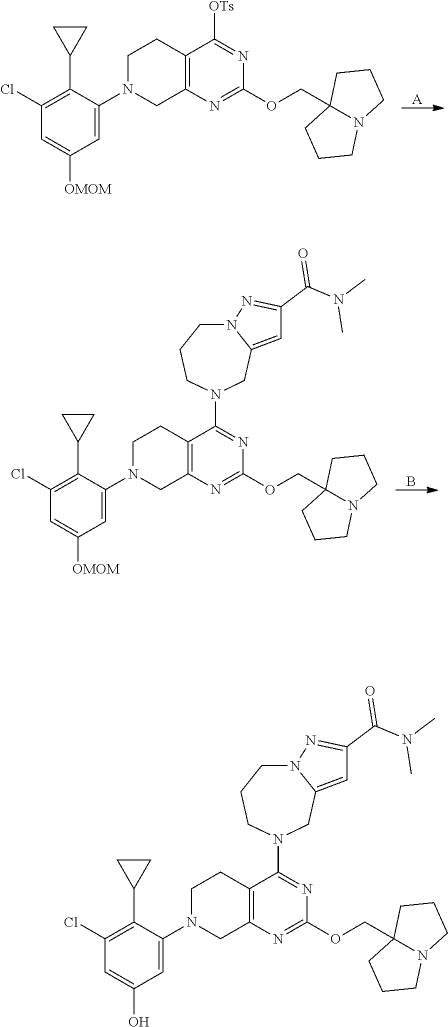

Synthesized according to Example 436. The title compound was obtained as red oil (FA salt). ¹H NMR (400 MHz, METHANOL-d₄) δ=8.54 (s, 1H), 6.59 (s, 1H), 6.54 (d, J=2.4 Hz, 1H), 6.37 (d, J=2.4 Hz, 1H), 4.56-4.47 (m, 2H), 4.29-4.21 (m, 2H), 4.12-4.02 (m, 4H), 3.45-3.37 (m, 2H), 3.34-3.31 (m, 2H), 3.31-3.28 (m, 5H), 3.10-2.97 (m, 5H), 2.91 (br s, 2H), 2.25-2.18 (m, 1H), 2.16 (br d, J=5.7 Hz, 2H), 2.14-2.11 (m, 1H), 2.11-2.05 (m, 2H), 2.05-1.97 (m, 2H), 1.95-1.87 (m, 2H), 1.70-1.60 (m, 1H), 1.05-0.96 (m, 2H), 0.72-0.65 (m, 2H); LCMS (ESI, M+1): m/z=647.5.

### Example 438

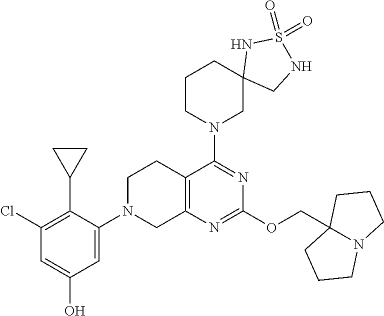 [SMILES: O=S1(=O)NCC2(CCCN(c3nc(OCC45CCCN4CCC5)nc4c3CCN(c3cc(O)cc(Cl)c3C3CC3)C4)C2)N1]

### 7-(7-(3-chloro-2-cyclopropyl-5-hydroxyphenyl)-2-((tetrahydro-1H-pyrrolizin-7a(5H)-yl)methoxy)-5,6,7,8-tetrahydropyrido[3,4-d]pyrimidin-4-yl)-2-thia-1,3,7-triazaspiro[4.5]decane 2,2-dioxide

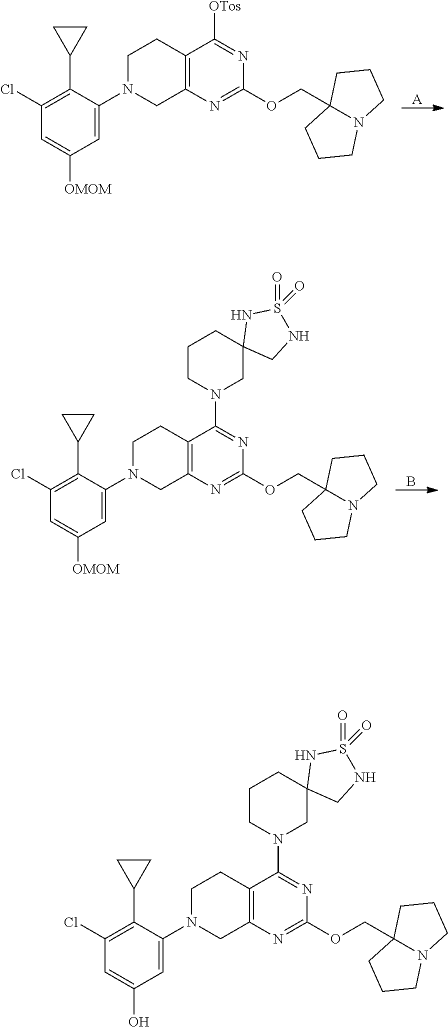

Synthesized according to Example 436. The title compound was obtained as white solid (FA salt). ¹H NMR (400 MHz, METHANOL-d₄) δ=8.55-8.53 (m, 1H), 6.54 (d, J=2.4 Hz, 1H), 6.40 (d, J=2.4 Hz, 1H), 4.52-4.39 (m, 2H), 4.18-4.10 (m, 1H), 4.09-3.99 (m, 2H), 3.84-3.75 (m, 1H), 3.64-3.55 (m, 2H), 3.54-3.47 (m, 1H), 3.45-3.33 (m, 3H), 3.24-3.14 (m, 4H), 2.95-2.81 (m, 2H), 2.34-2.23 (m, 2H), 2.22-2.08 (m, 4H), 2.06-1.96 (m, 3H), 1.91-1.80 (m, 2H), 1.79-1.73 (m, 1H), 1.69-1.59 (m, 1H), 1.06-0.95 (m, 2H), 0.73-0.63 (m, 2H); LCMS (ESI, M+1): m/z=630.3.

### Example 439

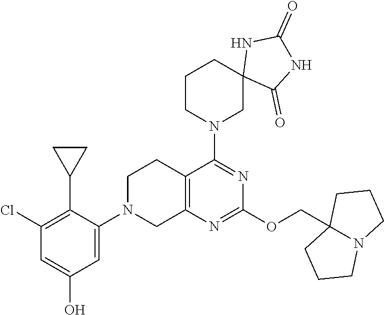 [SMILES: O=C1NC(=O)C2(CCCN(c3nc(OCC45CCCN4CCC5)nc4c3CCN(c3cc(O)cc(Cl)c3C3CC3)C4)C2)N1]

### 7-(7-(3-chloro-2-cyclopropyl-5-hydroxyphenyl)-2-((tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione

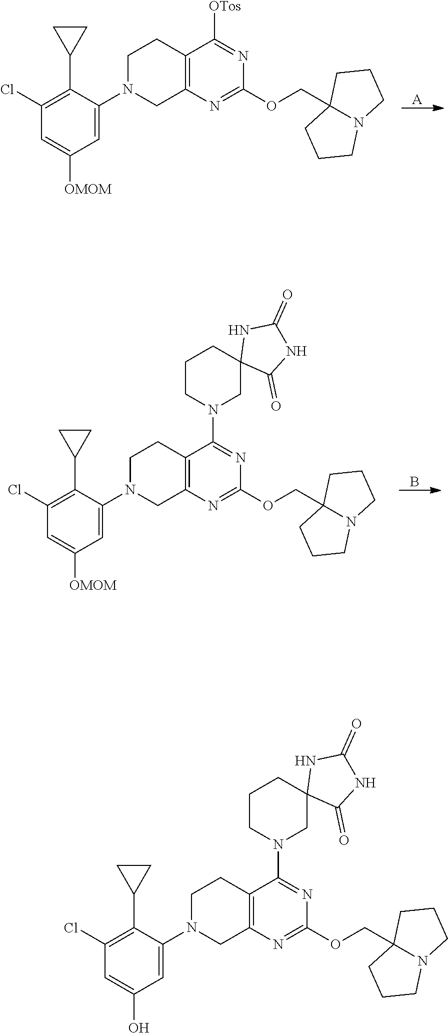

Synthesized according to Example 436. The title compound was obtained as a white solid (FA salt). ¹H NMR (400 MHz, CD3OD) δ=8.53 (s, 1H), 6.54 (d, J=2.4 Hz, 1H), 6.40 (d, J=2.4 Hz, 1H), 4.49-4.36 (m, 2H), 4.19 (br d, J=13.2 Hz, 1H), 4.10 (s, 2H), 4.07-4.00 (m, 1H), 3.65-3.55 (m, 2H), 3.40 (d, J=13.2 Hz, 1H), 3.30-3.26 (m, 2H), 3.25-3.14 (m, 3H), 2.99-2.79 (m, 2H), 2.31-1.83 (m, 12H), 1.70-1.61 (m, 1H), 1.05-0.93 (m, 2H), 0.68 (br d, J=3.6 Hz, 2H); LCMS (ESI, M+1): m/z=608.2.

### Example 440

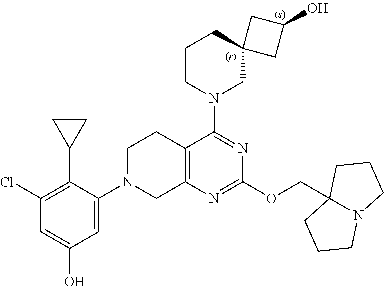 [SMILES: Oc1cc(Cl)c(C2CC2)c(N2CCc3c(nc(OCC45CCCN4CCC5)nc3N3CCC[C@]4(C3)C[C@H](O)C4)C2)c1]

### (2s,4r)-6-(7-(3-chloro-2-cyclopropyl-5-hydroxyphenyl)-2-((tetrahydro-1H-pyrrolizin-7a(5H)-yl)methoxy)-5,6,7,8-tetrahydropyrido[3,4-d]pyrimidin-4-yl)-6-azaspiro[3.5]nonan-2-ol

### Example 441

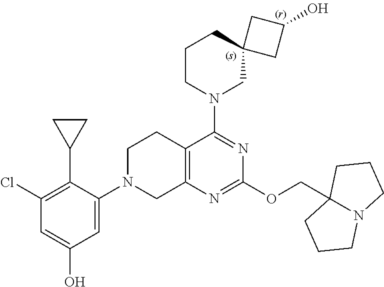 [SMILES: Oc1cc(Cl)c(C2CC2)c(N2CCc3c(nc(OCC45CCCN4CCC5)nc3N3CCC[C@]4(C3)C[C@@H](O)C4)C2)c1]

### (2r,4s)-6-(7-(3-chloro-2-cyclopropyl-5-hydroxyphenyl)-2-((tetrahydro-1H-pyrrolizin-7a(5H)-yl)methoxy)-5,6,7,8-tetrahydropyrido[3,4-d]pyrimidin-4-yl)-6-azaspiro[3.5]nonan-2-ol

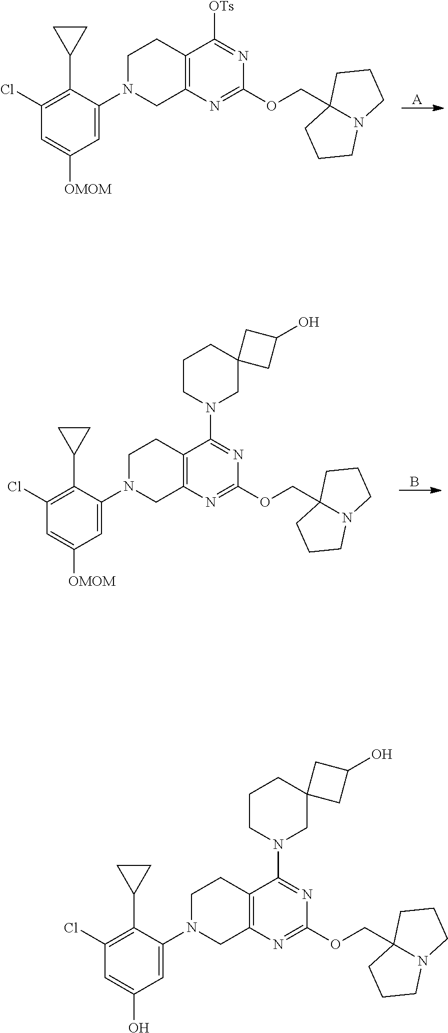

Synthesized according to Example 436. The two isomers were separated with SFC [column: DAICEL CHIRALPAK IG (250 mm×30 mm, 10 μm); mobile phase: [0.1% NH₃H₂O EtOH]; B %: 65%-65%, 5.2 minutes] to afford Example 440 as a white solid and Example 441 as a white solid.

Example 440: ¹H NMR (400 MHz, METHANOL-d₄) δ=8.54 (s, 1H), 6.54 (d, J=2.3 Hz, 1H), 6.42-6.37 (m, 1H), 4.29 (s, 2H), 4.27-4.18 (m, 1H), 4.11-4.01 (m, 2H), 3.52 (s, 4H), 3.40-3.33 (m, 2H), 3.30-3.21 (m, 2H), 3.04-2.93 (m, 2H), 2.89-2.79 (m, 2H), 2.30-2.21 (m, 2H), 2.20-2.12 (m, 2H), 2.10-1.95 (m, 4H), 1.94-1.84 (m, 2H), 1.72-1.59 (m, 7H), 1.04-0.94 (m, 2H), 0.71-0.64 (m, 2H);

Example 441: ¹H NMR (400 MHz, METHANOL-d₄) δ=8.56-8.51 (m, 1H), 6.54 (d, J=2.4 Hz, 1H), 6.40 (d, J=2.3 Hz, 1H), 4.34 (s, 2H), 4.28-4.21 (m, 1H), 4.06 (s, 2H), 3.55-3.52 (m, 2H), 3.48 (br s, 2H), 3.45-3.38 (m, 2H), 3.27 (br s, 2H), 3.07-2.99 (m, 2H), 2.86 (br s, 2H), 2.25-2.17 (m, 2H), 2.15-2.06 (m, 3H), 2.06-1.98 (m, 2H), 1.97-1.89 (m, 2H), 1.76-1.64 (m, 7H), 1.04-0.96 (m, 2H), 0.71-0.64 (m, 2H).LCMS (ESI, M+1): m/z=580.4

### Example 442

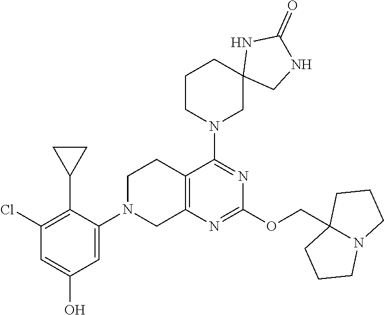 [SMILES: O=C1NCC2(CCCN(c3nc(OCC45CCCN4CCC5)nc4c3CCN(c3cc(O)cc(Cl)c3C3CC3)C4)C2)N1]

### 7-(7-(3-chloro-2-cyclopropyl-5-hydroxyphenyl)-2-((tetrahydro-1H-pyrrolizin-7a(5H)-yl)methoxy)-5,6,7,8-tetrahydropyrido[3,4-d]pyrimidin-4-yl)-1,3,7-triazaspiro[4.5]decan-2-one

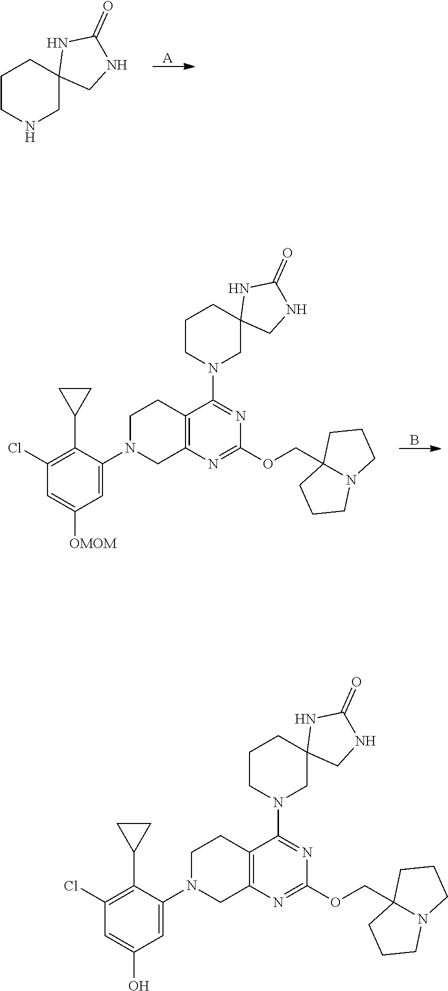

Synthesized according to Example 436. The title compound was obtained as white solid (FA salt). ¹H NMR (400 MHz, METHANOL-d₄) δ=8.65-8.39 (m, 1H), 6.55 (d, J=2.4 Hz, 1H), 6.39 (d, J=2.4 Hz, 1H), 4.53-4.42 (m, 2H), 4.19-4.10 (m, 1H), 4.08-3.99 (m, 1H), 3.77-3.63 (m, 3H), 3.62-3.46 (m, 3H), 3.43-3.34 (m, 2H), 3.30-3.22 (m, 3H), 3.22-3.13 (m, 1H), 3.00-2.90 (m, 1H), 2.86-2.75 (m, 1H), 2.35-2.25 (m, 2H), 2.24-2.11 (m, 4H), 2.11-2.03 (m, 2H), 1.86 (br s, 3H), 1.80-1.69 (m, 1H), 1.69-1.59 (m, 1H), 1.07-0.92 (m, 2H), 0.75-0.60 (m, 2H); LCMS (ESI, M+1): m/z=594.2.

### Example 443

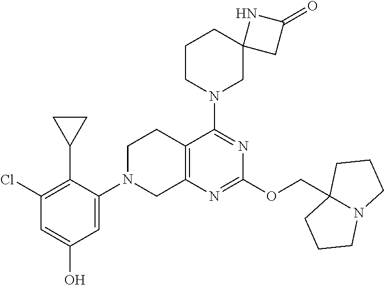 [SMILES: O=C1CC2(CCCN(c3nc(OCC45CCCN4CCC5)nc4c3CCN(c3cc(O)cc(Cl)c3C3CC3)C4)C2)N1]

### 6-(7-(3-chloro-2-cyclopropyl-5-hydroxyphenyl)-2-((tetrahydro-1H-pyrrolizin-7a(5H)-yl)methoxy)-5,6,7,8-tetrahydropyrido[3,4-d]pyrimidin-4-yl)-1,6-diazaspiro[3.5]nonan-2-one

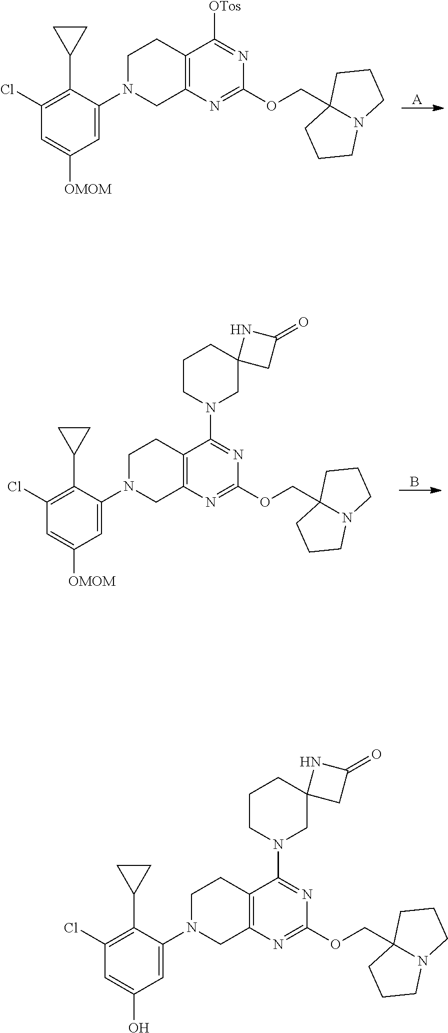

Synthesized according to Example 436. The title compound was obtained as a yellow gum (FA salt). ¹H NMR (400 MHz, DMSO-d₆) δ=8.42 (s, 1H), 8.21 (s, 1H), 6.50 (d, J=2.4 Hz, 1H), 6.37 (d, J=2.4 Hz, 1H), 3.99 (br d, J=9.2 Hz, 4H), 3.22 (br d, J=5.6 Hz, 2H), 3.07-2.97 (m, 3H), 2.75 (br s, 2H), 2.71-2.62 (m, 4H), 2.58 (s, 2H), 1.95-1.55 (m, 14H), 1.05-0.89 (m, 2H), 0.57 (br t, J=6.0 Hz, 2H); LCMS (ESI, M+1): m/z=579.4.

### Example 444

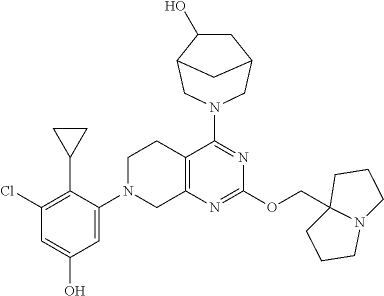 [SMILES: Oc1cc(Cl)c(C2CC2)c(N2CCc3c(nc(OCC45CCCN4CCC5)nc3N3CC4CC(O)C(C4)C3)C2)c1]

### 3-(7-(3-chloro-2-cyclopropyl-5-hydroxyphenyl)-2-((tetrahydro-1H-pyrrolizin-7a(5H)-yl)methoxy)-5,6,7,8-tetrahydropyrido[3,4-d]pyrimidin-4-yl)-3-azabicyclo[3.2.1]octan-6-ol

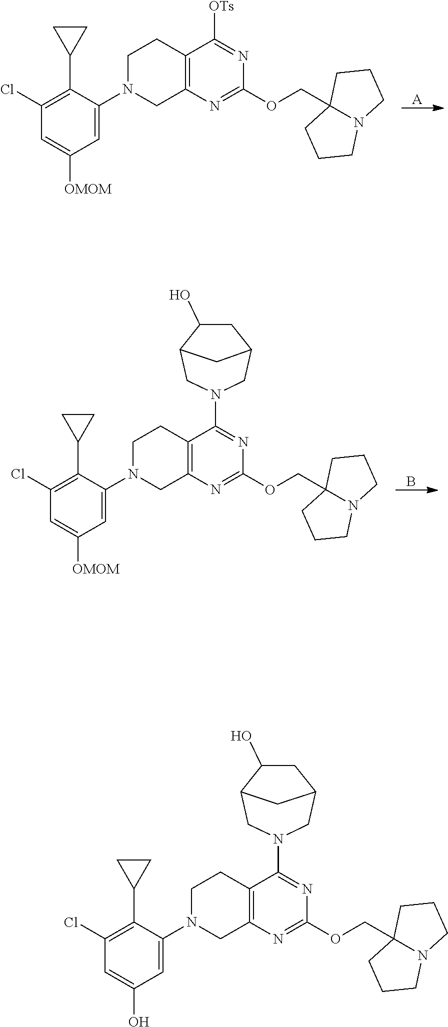

Synthesized according to Example 436. The title compound was obtained as white solid (FA salt). ¹H NMR (400 MHz, METHANOL-d₄) δ=8.54 (s, 1H), 6.53 (d, J=2.4 Hz, 1H), 6.39 (d, J=2.4 Hz, 1H), 4.47-4.27 (m, 4H), 4.13 (br d, J=12.4 Hz, 1H), 4.06 (s, 2H), 3.53-3.43 (m, 2H), 3.27-2.97 (m, 7H), 2.93-2.82 (m, 1H), 2.27-1.93 (m, 11H), 1.80-1.60 (m, 3H), 1.45-1.35 (m, 1H), 1.02-0.96 (m, 2H), 0.71-0.64 (m, 2H); LCMS (ESI, M+1): m/z=566.3.

### Example 445

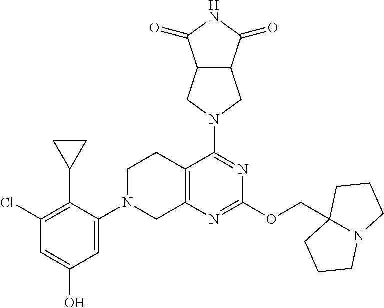 [SMILES: O=C1NC(=O)C2CN(c3nc(OCC45CCCN4CCC5)nc4c3CCN(c3cc(O)cc(Cl)c3C3CC3)C4)CC12]

### 5-(7-(3-chloro-2-cyclopropyl-5-hydroxyphenyl)-2-((tetrahydro-1H-pyrrolizin-7a(5H)-yl)methoxy)-5,6,7,8-tetrahydropyrido[3,4-d]pyrimidin-4-yl)tetrahydropyrrolo[3,4-c]pyrrole-1,3(2H,3aH)-dione

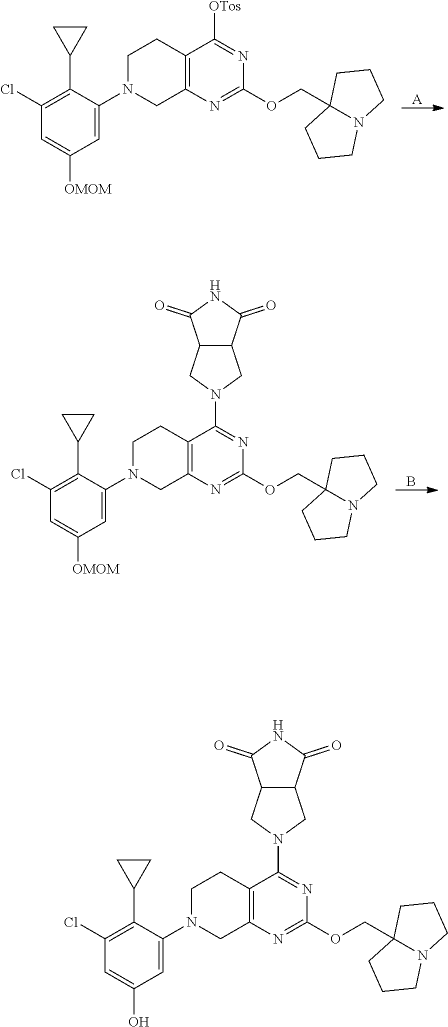

Synthesized according to Example 436. The title compound was obtained as white solid (FA salt). ¹H NMR (400 MHz, DMSO-d₆) δ=8.23 (s, 1H), 6.50 (d, J=2.4 Hz, 1H), 6.36 (d, J=2.0 Hz, 1H), 4.11 (br d, J=11.6 Hz, 2H), 4.00-3.94 (m, 4H), 3.60-3.55 (m, 2H), 3.45 (br d, J=6.8 Hz, 2H), 3.23 (br s, 2H), 3.06-3.00 (m, 2H), 2.76 (br s, 2H), 2.68-2.62 (m, 2H), 1.95-1.55 (m, 10H), 0.99-0.92 (m, 2H), 0.58-0.53 (m, 2H); LCMS (ESI, M+1): m/z=579.1.

### Example 446

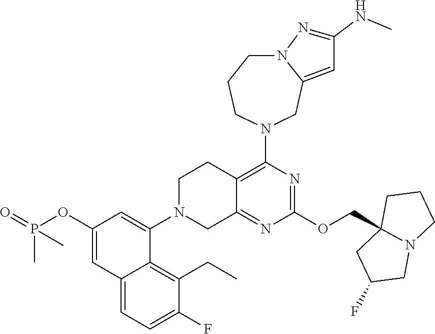 [SMILES: CCc1c(F)ccc2cc(OP(C)(C)=O)cc(N3CCc4c(nc(OC[C@@]56CCCN5C[C@H](F)C6)nc4N4CCCn5nc(NC)cc5C4)C3)c12]

### 5-ethyl-6-fluoro-4-(2-(((2R,7aS)-2-fluorohexahydro-1H-pyrrolizin-7a-yl)methoxy)-4-(2-(methylamino)-7,8-dihydro-4H-pyrazolo[1,5-a][1,4]diazepin-5(6H)-yl)-5,6-dihydropyrido[3,4-d]pyrimidin-7(8H)-yl)naphthalen-2-yl dimethylphosphinate

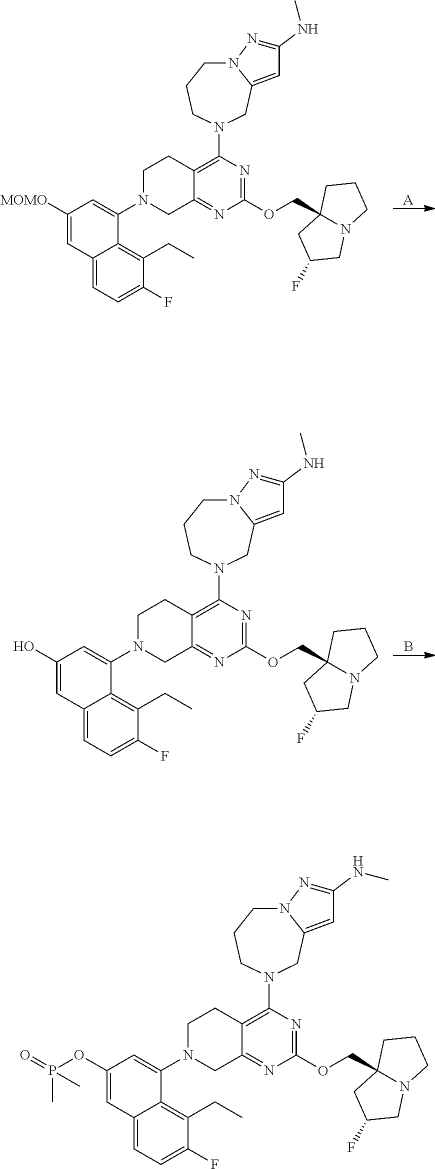

Step A. 5-ethyl-6-fluoro-4-(2-((2R,7aS)-2-fluorohexahydro-1H-pyrrolizin-7a-yl)methoxy)-4-(2-(methylamino)-7,8-dihydro-4H-pyrazolo[1,5-a][1,4]diazepin-5(6H)-yl)-5,6-dihydropyrido[3,4-d] pyrimidin-7(8H)-yl)naphthalen-2-ol: To a mixture of 5-(7-(8-ethyl-7-fluoro-3-(methoxymethoxy)naphthalen-1-yl)-2-(((2R,7aS)-2-fluorotetrahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepin-2-amine (300 mg, 1.0 equiv) in ACN (1.0 mL) was added HCl-MeOH (2.0 mL, 18 equiv). The reaction was stirred at 25° C. for 0.5 hours. The mixture was concentrated and purified by reversed phase flash chromatography [C18, 0.1% formic acid condition] to afford the title compound (180 mg, 60% yield) as yellow solid; LCMS (ESI, M+1): m/z=645.4.

Step B. 5-ethyl-6-fluoro-4-(2-(((2R,7aS)-2-fluorohexahydro-1H-pyrrolizin-7a-yl)methoxy)-4-(2-(methylamino)-7,8-dihydro-4H-pyrazolo[1,5-a][1,4]diazepin-5(6H)-yl)-5,6-dihydropyrido[3,4-d]pyrimidin-7(8H)-yl)naphthalen-2-yl dimethylphosphinate: To a mixture of 5-ethyl-6-fluoro-4-(2-(((2R,7aS)-2-fluorohexahydro-1H-pyrrolizin-7a-yl)methoxy)-4-(2-(methylamino)-7,8-dihydro-4H-pyrazolo[1,5-a][1,4]diazepin-5(6H)-yl)-5,6-dihydropyrido[3,4-d]pyrimidin-7(8H)-yl)naphthalen-2-ol (49.0 mg, 1.0 equiv) in DCM (0.5 mL) were added N,N-diethylpropan-2-amine (98.2 mg, 10 equiv) and dimethylphosphinic chloride (6.84 mg, 0.8 equiv). The reaction was stirred at 20° C. for 2 hours. The mixture was concentrated, purified by prep-HPLC [Waters xbridge C18 150×25 mm×10 μm; A: water (NH₄HCO₃), B: ACN; B %: 30%-60% over 8 min] and lyophilized to afford the title compound (3.43 mg, 5.9% yield) as pink solid. ¹H NMR (400 MHz, dimethylsulfoxide-d₆) δ=7.83 (dd, J=6.0, 9.2 Hz, 1H), 7.58 (s, 1H), 7.41 (t, J=9.6 Hz, 1H), 7.25 (d, J=1.6 Hz, 1H), 5.40-5.35 (m, 1H), 5.32-5.14 (m, 1H), 4.97-4.80 (m, 2H), 4.56 (br dd, J=2.8, 16.4 Hz, 1H), 4.27-4.06 (m, 3H), 3.95-3.86 (m, 2H), 3.81 (dd, J=8.0, 10.0 Hz, 1H), 3.73-3.61 (m, 2H), 3.49-3.41 (m, 1H), 3.18-3.10 (m, 2H), 3.04 (br s, 2H), 3.01-2.95 (m, 1H), 2.84-2.76 (m, 1H), 2.69-2.63 (m, 1H), 2.58 (br d, J=4.0 Hz, 3H), 2.19-2.09 (m, 1H), 2.08-2.00 (m, 1H), 2.00-1.78 (m, 5H), 1.78-1.69 (m, 2H), 1.65 (d, J=14.4 Hz, 6H), 1.09 (t, J=7.2 Hz, 3H); ¹⁹F NMR (400 MHz, dimethylsulfoxide-d₆) δ=−117.498, -172.045; LCMS (ESI, M+1): m/z=721.5.

### Example 447

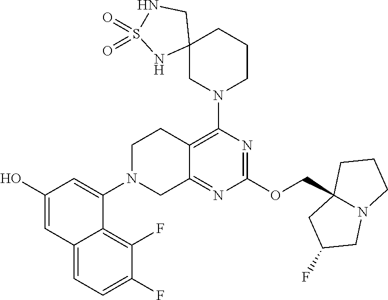 [SMILES: O=S1(=O)NCC2(CCCN(c3nc(OC[C@@]45CCCN4C[C@H](F)C5)nc4c3CCN(c3cc(O)cc5ccc(F)c(F)c35)C4)C2)N1]

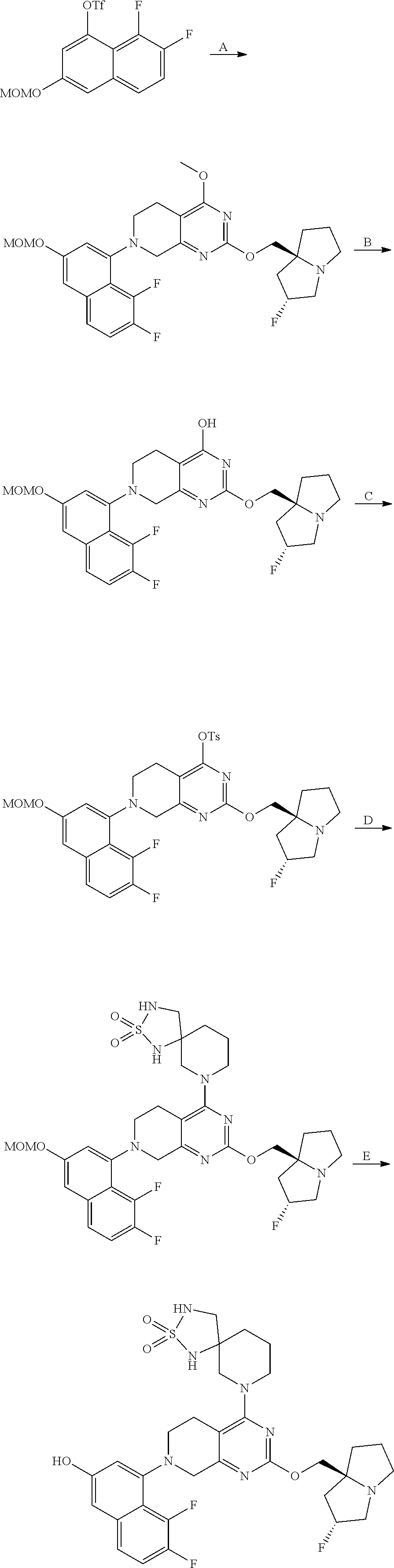

### 7-(7-(7,8-difluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-2-thia-1,3,7-triazaspiro[4.5]decane 2,2-dioxide

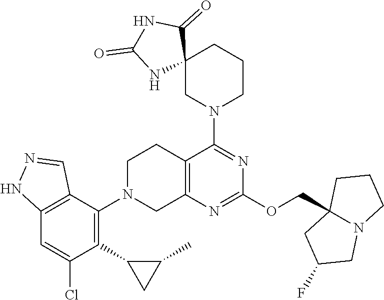 [SMILES: C[C@@H]1C[C@@H]1c1c(Cl)cc2[nH]ncc2c1N1CCc2c(nc(OC[C@@]34CCCN3C[C@H](F)C4)nc2N2CCC[C@]3(C2)NC(=O)NC3=O)C1]

Step A 7-(7.8-difluoro-3-(methoxymethoxy)naphthalen-1-yl)-2-(((2R,7aS)-2-fluorohexahydro-1H-pyrrolizin-7a-yl)methoxy)-4-methoxy-5,6,7,8-tetrahydropyrido[3,4-d] pyrimidine: To a solution of 2-(((2R,7aS)-2-fluorotetrahydro-1H-pyrrolizin-7a(5H)-yl)methoxy)-4-methoxy-5,6,7,8-tetrahydropyrido[3,4-d]pyrimidine (500 mg, 1.0 equiv) and [7,8-difluoro-3-(methoxymethoxy)-1-naphthyl]trifluoromethanesulfonate (866 mg, 1.5 equiv) in toluene (5 mL) were added Xantphos (179 mg, 0.20 equiv), (1E,4E)-1,5-diphenylpenta-1,4-dien-3-one; palladium (142 mg, 0.10 equiv), and Cs₂CO₃ (1.52 g, 3.0 equiv). The mixture was degassed and purged with nitrogen 3 times. The reaction was stirred at 100° C. for 12 hours. The mixture was diluted with H₂O (5 mL) and extracted with EtOAc (3×5 mL). The combined organic layers were washed with brine (5 mL), dried over anhydrous sodium sulfate, concentrated, and purified by reversed phase HPLC (0.1% FA condition) to afford the title compound (430 mg, 51% yield) as yellow solid; LCMS (ESI, M+1): m/z=545.4.

Step B. 7-(7,8-difluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-ol: To a mixture of EtSH (0.51 g, 15 equiv) in DMAc (3 mL) was added NaH (44.1 mg, 60% purity, 2.0 equiv) at 0° C. The reaction was stirred at 0° C. for 0.5 hours. 7-(7,8-difluoro-3-(methoxymethoxy)naphthalen-1-yl)-2-(((2R,7aS)-2-fluorohexahydro-1H-pyrrolizin-7a-yl)methoxy)-4-methoxy-5,6,7,8-tetrahydropyrido[3,4-d]pyrimidine (300 mg, 1.0 equiv) was added to the mixture at 0° C. The reaction was stirred at 60° C. for another 1 hour. The mixture was diluted with water (4 mL) and the pH was adjusted to 6 with HCl (2 M) aqueous solution. The aqueous solution was extracted with EtOAc (3×4 mL). The organic layers were washed with brine (2×4 mL), dried over anhydrous sodium sulfate. concentrated, and purified by reversed phase HPLC (0.1% FA condition) to afford the title compound (200 mg, 60% yield) as yellow solid; LCMS (ESI, M+1): m/z=531.2.

Step C. 7-(7,8-difluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl 4-methylbenzenesulfonate: To a mixture of 7-(7,8-difluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-ol (200 mg, 1.0 equiv), N,N-diethylpropan-2-amine (146 mg, 3.0 equiv) and DMAP (4.61 mg, 0.1 equiv) in DCM (2 mL) was added TosCl (180 mg, 2.5 equiv) at 0° C. The reaction was stirred at 25° C. for 0.5 hours. The reaction was concentrated and purified by column chromatography (Al₂O₃, petroleum ether/ethyl acetate=10/1 to 0/1) to afford the title compound (100 mg, 35% yield) as yellow soild; LCMS (ESI, M+1): m/z=685.5.

Step D. 7-(7-(7,8-difluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-2-thia-1,3,7-triazaspiro[4.5]decane 2,2-dioxide: To a solution of 7-(7,8-difluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (90.0 mg, 1.0 equiv) in DMF (1 mL) were added N,N-diethylpropan-2-amine (136 mg, 8.0 equiv), 4A molecular sieve (5.00 mg, 1.0 equiv), and 2-thia-1,3,7-triazaspiro[4.5]decane 2,2-dioxide (50.3 mg, 2.0 equiv). The reaction was stirred at 40° C. for 12 hours. The mixture was filtered and purified by reversed phase HPLC (0.1% FA condition) to afford the title compound (90.0 mg, 91% yield) as yellow solid; LCMS (ESI, M+1): m/z=704.3.

Step E. 7-(7-(7,8-difluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-2-thia-1,3,7-triazaspiro[4.5]decane 2,2-dioxide: A solution of 7-(7-(7,8-difluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-2-thia-1,3,7-triazaspiro[4.5]decane 2,2-dioxide (50.0 mg, 1.0 equiv) in HCl•MeOH (4 M, 0.5 mL, 1.0 equiv) was stirred at 20° C. for 0.5 hours. The reaction was concentrated under reduced pressure to remove solvent. The pH of residue was adjusted to 8 with Na₂CO₃ aqueous solution. The mixture was extracted with EtOAc (3×2 mL). The combined organic layers were washed with brine (3×2 mL), dried over anhydrous sodium sulfate, concentrated, and purified by prep-HPLC (column: Welch Xtimate C18 150×25 mm×5 μm; mobile phase: [water (NH₃•H₂O)—ACN]; B %: 30%-60%, 8 min) to afford the title compound (57.0 mg, 81% yield) as pink solid; ¹H NMR (400 MHz, METHANOL-d₄) δ=7.43 (dd, J=4.8, 9.2 Hz, 1H), 7.30 (dt, J=7.6, 9.2 Hz, 1H), 6.85 (br s, 2H), 5.39-5.19 (m, 1H), 4.67-4.51 (m, 1H), 4.27-4.10 (m, 3H), 3.93 (br d, J=16.0 Hz, 2H), 3.70-3.39 (m, 4H), 3.28-3.14 (m, 5H), 3.07-2.83 (m, 2H), 2.73-2.57 (m, 1H), 2.33-2.07 (m, 3H), 2.03-1.91 (m, 4H), 1.90-1.73 (m, 3H); LCMS (ESI, M+1): m/z=660.3.

### Example 448

### (R)-7-(7-(6-chloro-5-((1S,2R)-2-methylcyclopropyl)-1H-indazol-4-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione

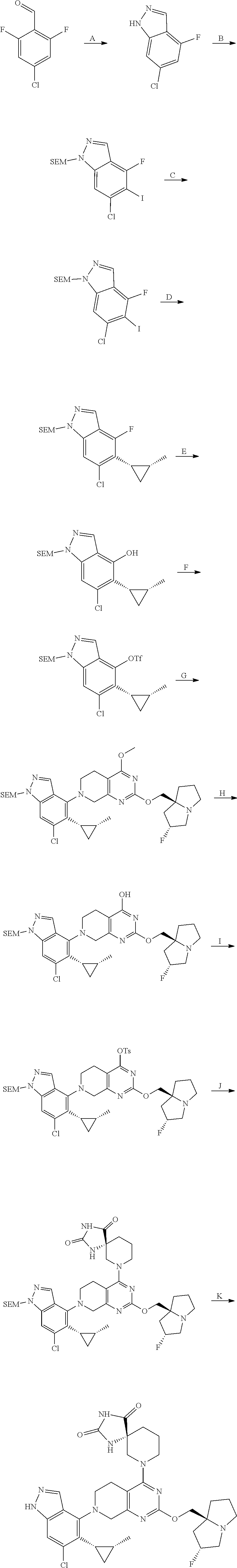

Step A. 6-chloro-4-fluoro-1H-indazole: To a solution of 4-chloro-2,6-difluoro-benzaldehyde (100 g, 1.0 equiv) in dioxane (1.0 L) was added N2H4 H₂O (58.1 g, 2.0 equiv) dropwise at 25° C. over 10 minutes. The mixture was stirred at 25° C. for 0.5 hours, and at 95° C. for 15.5 hours. The reaction was diluted with H₂O (500 mL) at 25° C. and extracted with EtOAc (2×500 mL). The combined organic layers were washed with brine (200 mL), dried over anhydrous sodium sulfate, and concentrated to afford the title compound (95.0 g, crude) as a white solid; ¹H NMR (400 MHz, DMSO-d₆) δ=14.08-12.38 (m, 1H), 8.23 (d, J=0.4 Hz, 1H), 7.51 (s, 1H), 7.06 (dd, J=1.2, 9.6 Hz, 1H); LCMS (ESI, M+1): m/z=171.0.

Step B. 6-chloro-4-fluoro-1-((2-(trimethylsilyl)ethoxy)methyl)-1H-indazole: To a solution of 6-chloro-4-fluoro-1H-indazole (40.0 g, 1.0 equiv) in THF (200 mL) was added NaH (14.1 g, 1.5 equiv) in portions at 0° C. for 30 minutes. The mixture was stirred at 25° C. for 0.5 hours. Then SEM-C1 (46.9 g, 1.2 equiv) was added to the mixture dropwise at 0° C. for 20 minutes. The mixture was stirred at 25° C. for 1 hour. The mixture was quenched with H₂O (300 mL) in dropwise addition at 0° C. for 30 minutes. The mixture was extracted with EtOAc (2×300 mL). The combined organic layers were dried over anhydrous sodium sulfate, concentrated, and purified by column chromatography [SiO₂, petroleum ether/ethyl acetate=I/O to 10/1] to afford the title compound (42.0 g, 60% yield) as yellow solid; ¹H NMR (400 MHz, CDCl₃-d) δ=8.09-8.00 (m, 1H), 7.45-7.37 (m, 1H), 6.88 (dd, J=1.2, 9.6 Hz, 1H), 5.70 (s, 2H), 3.63-3.46 (m, 2H), 0.96-0.84 (m, 2H), −0.01-0.06 (m, 9H); LCMS (ESI, M+1): m/z=301.3.

Step C. 6-chloro-4-fluoro-5-iodo-1-((2-(trimethylsilyl)ethoxy)methyl)-1H-indazole: To a solution of 2-[(6-chloro-4-fluoro-indazol-1-yl)methoxy]ethyl-trimethyl-silane (20.0 g, 1.0 equiv) in THF (100 mL) was added LDA (43.2 mL, 1.3 equiv) dropwise at −65° C. for 5 minutes. The mixture was stirred at −65° C. for 55 minutes. Then a solution of I₂ (21.9 g, 1.3 equiv) in THE (50.0 mL) was added to the mixture slowly for 15 minutes and stirred at −65° C. for 1 hour. The mixture was quenched with H₂O (100 mL) at 0° C. for 15 minutes. The mixture was extracted with EtOAc (2×100 mL). The combined organic layers were dried over anhydrous sodium sulfate, concentrated, and purified by reversed phase flash chromatography [C18, 0.1% formic acid condition] to afford the title compound (12.0 g, 42% yield) as yellow solid; ¹H NMR (400 MHz, CDCl₃-d) δ=8.03 (s, 1H), 7.61 (s, 1H), 5.70 (s, 2H), 3.58-3.50 (m, 2H), 0.93-0.86 (m, 2H), −0.04 (s, 9H). LCMS (ESI, M+1): m/z=427.2.

Step D. 6-chloro-4-fluoro-5-((1S,2R)-2-methylcyclopropyl)-1-((2-(trimethylsilyl)ethoxy)methyl)-1H-indazole: To a solution of 6-chloro-4-fluoro-5-iodo-1-((2-(trimethylsilyl)ethoxy)methyl)-1H-indazole (2.0 g, 1.0 equiv) and 4,4,5,5-tetramethyl-2-(2-methylcyclopropyl)-1,3,2-dioxaborolane (1.02 g, 1.2 equiv) in dioxane (20 mL) were added K₃PO₄ (1.5 M, 9.37 mL, 3.0 equiv) and Pd(dppf)Cl₂ (343 mg, 0.1 equiv) under N₂. The mixture was stirred at 100° C. for 10 hours. The mixture was diluted with H₂O (50 mL) and extracted with EtOAc (2×20 mL). The combined organic layers were dried over anhydrous sodium sulfate, concentrated, and purified by reversed phase flash chromatography [C18, 0.1% formic acid condition] to afford the title compound (1.0 g, 60% yield) as yellow liquid; LCMS (ESI, M+1): m/z=355.1.

Step E. 6-chloro-5-((1S,2R)-2-methylcyclopropyl)-1-((2-(trimethylsilyl)ethoxy)methyl)-1H-indazol-4-ol: To a solution of 2-[[6-chloro-4-fluoro-5-(2-methylcyclopropyl)indazol-1-yl]methoxy]ethyl-trimethyl-silane (1.0 g, 1.0 equiv) and 2-methylsulfonylethanol (5.25 g, 15 equiv) in THE (4 mL) was added NaH (1.69 g, 60% purity, 15 equiv) at 0° C. The mixture was stirred at 40° C. for 12 hours. The mixture was quenched with H₂O (10 mL) in dropwise addition at 0° C. for 15 minutes. The mixture was extracted with EtOAc (3×10 mL). The combined organic layers were dried over anhydrous sodium sulfate, concentrated, and purified by reversed phase flash chromatography [C18, 0.1% formic acid condition] to afford the title compound (0.79 g, 79% yield) as yellow liquid; LCMS (ESI, M+1): m/z=353.1.

Step F. 6-chloro-5-((1S,2R)-2-methylcyclopropyl)-1-((2-(trimethylsilyl)ethoxy)methyl)-1H-indazol-4-yl trifluoromethanesulfonate: To a solution of 6-chloro-5-((1S,2R)-2-methylcyclopropyl)-1-((2-(trimethylsilyl)ethoxy)methyl)-1H-indazol-4-ol (0.79 g, 1.0 equiv) in DCM (10 mL) were added N,N-diethylpropan-2-amine (579 mg, 2.0 equiv) and Tf₂O (947 mg, 1.5 equiv) at −40° C. The mixture was stirred at −40° C. for 0.5 hours. The mixture was diluted with H₂O (10 mL) and extracted with DCM (2×10 mL). The combined organic layers were dried over anhydrous sodium sulfate, concentrated, and purified by column chromatography [SiO₂, petroleum ether/ethyl acetate=100/1 to 10/1] to afford the title compound (0.71 g, 63% yield) as yellow liquid; LCMS (ESI, M+1): m/z=485.1.

Step G. 7-(6-chloro-5-((1S,2R)-2-methylcyclopropyl)-1-((2-(trimethylsilyl)ethoxy)methyl)-1H-indazol-4-yl)-2-(((2R,7aS)-2-fluorohexahydro-1H-pyrrolizin-7a-yl)methoxy)-4-methoxy-5,6,7,8-tetrahydropyrido[3,4-d]pyrimidine: To a mixture of 2-(((2R,7aS)-2-fluorohexahydro-1H-pyrrolizin-7a-yl)methoxy)-4-methoxy-5,6,7,8-tetrahydropyrido[3,4-d]pyrimidine (500 mg, 1.0 equiv), 6-chloro-5-((1S,2R)-2-methylcyclopropyl)-1-((2-(trimethylsilyl)ethoxy)methyl)-1H-indazol-4-yl trifluoromethanesulfonate (72 mg, 1.0 equiv), and Cs₂CO₃ (1.52 g, 3.0 equiv) in dioxane (5 mL) was added Xantphos-Pd-G4 (149 mg, 0.10 equiv). The mixture was degassed and purged with N₂ 3 times, and then the mixture was stirred at 90° C. for 16 hours under N₂ atmosphere. The mixture was diluted with water (10 mL) and extracted with EtOAc (3×10 mL). The combined organic layers were dried over anhydrous sodium sulfate, concentrated, and purified by column chromatography [SiO₂, Petroleum ether/Ethyl acetate=100/1 to 0/1] and reversed phase flash chromatography [C18, 0.1% formic acid condition] to afford the title compound (160 mg, 15% yield) as white solid; LCMS (ESI, M+1): m/z=657.2.

Step H. 7-(6-chloro-5-((1S,2R)-2-methylcyclopropyl)-1-((2-(trimethylsilyl)ethoxy)methyl)-1H-indazol-4-yl)-2-(((2R,7aS)-2-fluorohexahydro-1H-pyrrolizin-7a-yl)methoxy)-5,6,7,8-tetrahydropyrido[3,4-d] pyrimidin-4-ol: To a solution of EtSH (104 mg, 10 equiv) in DMAC (1 mL) was added NaH (33.5 mg, 60% purity, 5.0 equiv) at 10° C. The mixture was stirred at 10° C. for 0.5 hours. Then 7-(6-chloro-5-((1S,2R)-2-methylcyclopropyl)-1-((2-(trimethylsilyl)ethoxy)methyl)-1H-indazol-4-yl)-2-(((2R,7aS)-2-fluorohexahydro-1H-pyrrolizin-7a-yl)methoxy)-4-methoxy-5,6,7,8-tetrahydropyrido[3,4-d]pyrimidine (110 mg, 1.0 equiv) was added to the above mixture. The reaction was stirred at 60° C. for 1 hour. The mixture was diluted with water (10 mL) and extracted with EtOAc (3×10 mL). The combined organic layers were washed with brine (3×30 mL), dried over anhydrous sodium sulfate, and concentrated to afford the title compound (170 mg, crude) as yellow liquid; LCMS (ESI, M+1): m/z=643.4.

Step I. 7-(6-chloro-5-((1S,2R)-2-methylcyclopropyl)-1-((2-(trimethylsilyl)ethoxy)methyl)-1H-indazol-4-yl)-2-(((2R,7aS)-2-fluorohexahydro-1H-pyrrolizin-7a-yl)methoxy)-5,6,7,8-tetrahydropyrido[3,4-d] pyrimidin-4-yl 4-methylbenzenesulfonate: To a solution of 7-(6-chloro-5-((1S,2R)-2-methylcyclopropyl)-1-((2-(trimethylsilyl)ethoxy)methyl)-1H-indazol-4-yl)-2-(((2R,7aS)-2-fluorohexahydro-1H-pyrrolizin-7a-yl)methoxy)-5,6,7,8-tetrahydropyrido[3,4-d]pyrimidin-4-ol (170 mg, 1.0 equiv), N,N-diethylpropan-2-amine (102 mg, 3.0 equiv), and DMAP (3.23 mg, 0.10 equiv) in DCM (2 mL) was added TosCl (75.6 mg, 1.5 equiv) at 0° C. The mixture was stirred at 20° C. for 2 hours. The mixture was diluted with water (10 mL) and extracted with DCM (3×10 mL). The combined organic layers were dried over anhydrous sodium sulfate, concentrated, and purified by column chromatography [Al₂O₃, Petroleum ether/Ethyl acetate=10/1 to 0/1] to afford the title compound (120 mg, 55% yield) as yellow solid; LCMS (ESI, M+1): m/z=797.5.

Step J. (R)-7-(7-(6-chloro-5-((1S,2R)-2-methylcyclopropyl)-1-((2-(trimethylsilyl)ethoxy)methyl)-1H-indazol-4-yl)-2-(((2R,7aS)-2-fluorohexahydro-1H-pyrrolizin-7a-yl)methoxy)-5,6,7,8-tetrahydropyrido[3,4-d] pyrimidin-4-yl)-1,3,7-triazaspiro[4.5]decane-2,4-dione: To a solution of 7-(6-chloro-5-((1S,2R)-2-methylcyclopropyl)-1-((2-(trimethylsilyl)ethoxy)methyl)-1H-indazol-4-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (120 mg, 1.0 equiv), (R)-1,3,7-triazaspiro[4.5]decane-2,4-dione (127 mg, 5.0 equiv), and 4 Å molecular sieve (50.0 mg) in DMF (0.5 mL) was added N,N-diethylpropan-2-amine (58.3 mg, 3.0 equiv). The mixture was stirred at 40° C. for 12 hours. The residue was filtered, washed with DMF (1 mL), and purified by reversed phase flash chromatography [C18, 0.1% formic acid condition] to afford the title compound (90.0 mg, 74% yield) as yellow solid; LCMS (ESI, M+1): m/z=794.3.

Step K. (R)-7-(7-(6-chloro-5-((1S,2R)-2-methylcyclopropyl)-1H-indazol-4-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione: To a solution of (R)-7-(7-(6-chloro-5-((1S,2R)-2-methylcyclopropyl)-1-((2-(trimethylsilyl)ethoxy)methyl)-1H-indazol-4-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione (80.0 mg, 1.0 equiv) in DCM (0.5 mL) was added TFA (1.54 g, 134 equiv). The mixture was stirred at 20° C. for 0.5 hours. The reaction was concentrated, purified with prep-HPLC [Phenomenex C18 150×25 mm×10 μm; A: water (FA), B: ACN; B %: 21%-41% over 10 min] and lyophilized to afford the title compound (4.03 mg) as white solid (FA salt). ¹H NMR (400 MHz, METHANOL-d₄) δ=8.07 (d, J=3.6 Hz, 1H), 7.41-7.21 (m, 1H), 5.47-5.23 (m, 1H), 4.49-4.30 (m, 2H), 4.27-4.10 (m, 3H), 4.08-3.96 (m, 1H), 3.76 (br s, 1H), 3.45-3.37 (m, 3H), 3.22-3.05 (m, 2H), 2.99-2.79 (m, 2H), 2.45-2.13 (m, 4H), 2.13-1.82 (m, 8H), 1.45-1.27 (m, 2H), 1.17 (br dd, J=6.0, 8.4 Hz, 2H), 1.10-0.98 (m, 1H), 0.95-0.83 (m, 1H), 0.80-0.71 (m, 1H); LCMS (ESI, M+1): m/z=664.3.

### Example 449

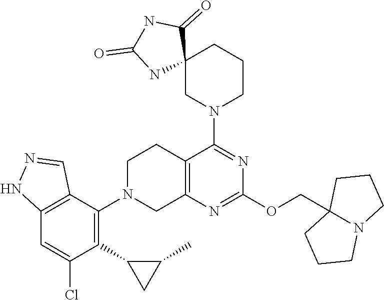 [SMILES: C[C@@H]1C[C@@H]1c1c(Cl)cc2[nH]ncc2c1N1CCc2c(nc(OCC34CCCN3CCC4)nc2N2CCC[C@]3(C2)NC(=O)NC3=O)C1]

### (R)-7-(7-(6-chloro-5-((1S,2R)-2-methylcyclopropyl)-1H-indazol-4-yl)-2-((hexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione

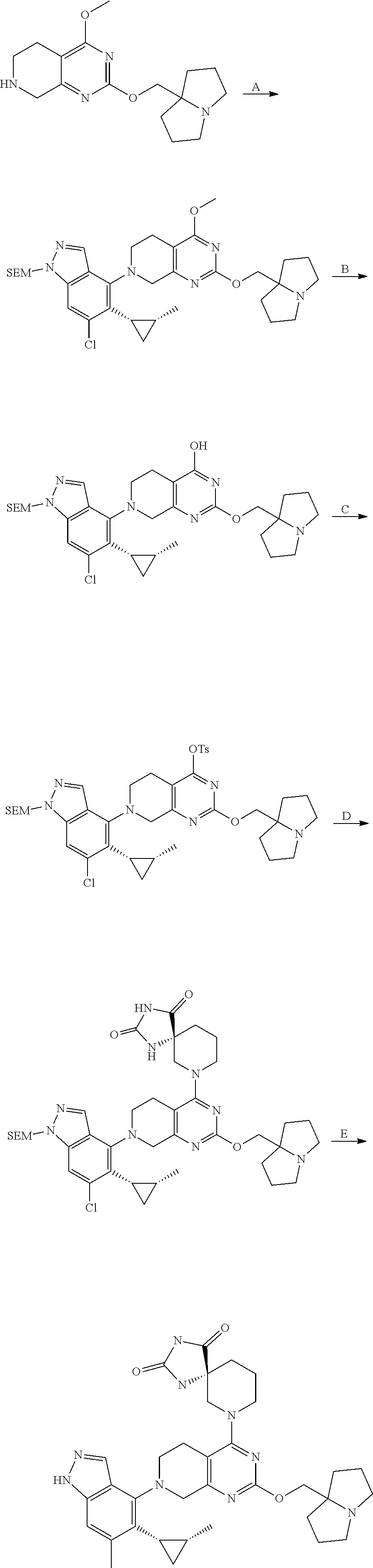

Synthesized according to Example (step GT-K) except that 2-((hexahydro-1H-pyrrolizin-7a-yl)methoxy)-4-methoxy-5,6,7,8-tetrahydropyrido[3,4-d]pyrimidine instead of 2-(((2R,7aS)-2-fluorohexahydro-1H-pyrrolizin-7a-yl)methoxy)-4-methoxy-5, 6,7, 8-tetrahydropyri do[3,4-d]pyrimidine was used in the first step. The title compound was obtained as white solid. ¹H NMR (400 MHz, methanol-d₄) δ=8.07 (br d, J=8.0 Hz, 1H), 7.32-7.29 (m, 1H), 4.38-4.28 (m, 2H), 4.19-4.12 (m, 2H), 4.11-3.98 (m, 2H), 3.83-3.62 (m, 2H), 3.42-3.35 (m, 1H), 3.17-3.02 (m, 3H), 2.96-2.80 (m, 2H), 2.72-2.65 (m, 2H), 2.07-2.00 (m, 3H), 1.95-1.81 (m, 7H), 1.78-1.67 (m, 2H), 1.45-1.36 (m, 1H), 1.20-1.14 (m, 2H), 1.12-0.63 (m, 4H); LCMS (ESI, M+1): m/z=646.2.

### Example 450

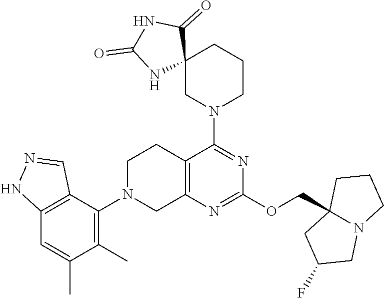 [SMILES: Cc1cc2[nH]ncc2c(N2CCc3c(nc(OC[C@@]45CCCN4C[C@H](F)C5)nc3N3CCC[C@]4(C3)NC(=O)NC4=O)C2)c1C]

### (R)-7-(7-(5,6-dimethyl-1H-indazol-4-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-triazaspiro[4.5m] odecane-2,4-dione

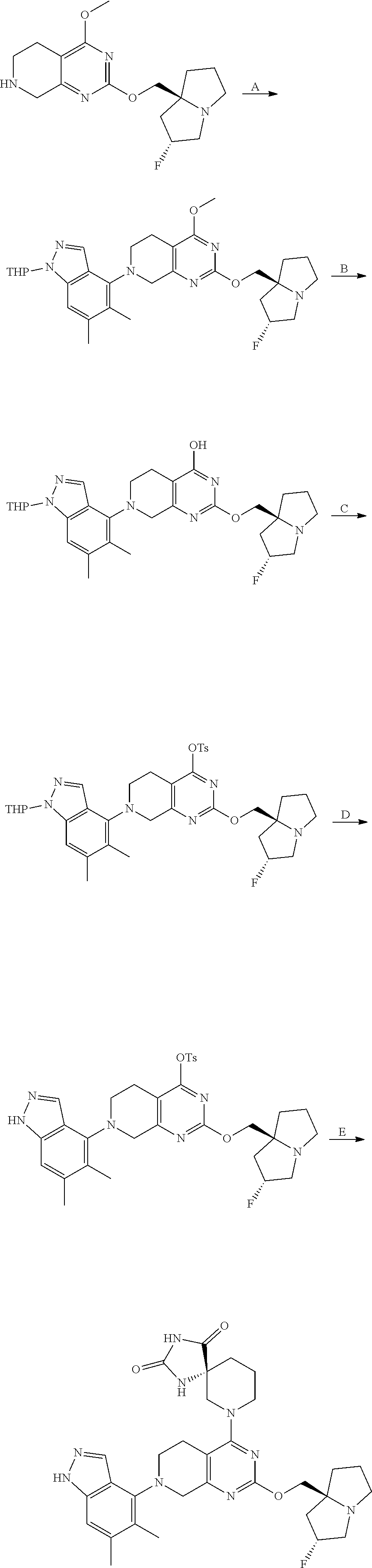

Step A. 7-(5,6-dimethyl-1-(tetrahydro-2H-pyran-2-yl)-1H-indazol-4-yl)-2-(((2R,7aS)-2-fluorohexahydro-11H-pyrrolizin-7a-yl)methoxy)-4-methoxy-5,6,7,8-tetrahydropyrido[3,4-d]pyrimidine: To a solution of 2-(((2R,7aS)-2-fluorohexahydro-1H-pyrrolizin-7a-yl)methoxy)-4-methoxy-5,6,7,8-tetrahydropyrido[3,4-d]pyrimidine (2.6 g, 1.0 equiv), 4-bromo-5,6-dimethyl-1-tetrahydropyran-2-yl-indazole (2.74 g, 1.1 equiv), CS₂CO₃ (7.88 g, 3.0 equiv), and RuPhos (941 mg, 0.25 equiv) in toluene (35 mL) was added Pd₂(dba)₃ (1.11 g, 0.15 equiv). The suspension was degassed under vacuum and purged with N₂ several times. The mixture was stirred at 110° C. for 14 hours. The mixture was filtered. The solution was diluted with water (100 mL) and extracted with ethyl acetate (4×60 mL). The organic phase was dried over Na₂SO₄, concentrated, and purified by reversed phase flash chromatography {0.1% FA condition] to afford the title compound (1.65 g, 37% yield, over 2 steps) as brown solid; LCMS (ESI, M+1): m/z=551.4.

Step B. 7-(5,6-dimethyl-1-(tetrahydro-2H-pyran-2-yl)-1H-indazol-4-yl)-2-(((2R,7aS)-2-fluorohexahydro-1H-pyrrolizin-7a-yl)methoxy)-5,6,7,8-tetrahydropyrido[3,4-d] pyrimidin-4-ol: To a mixture of NaH (219 mg, 60% purity, 2.0 equiv) in DMAC (20 mL) was added EtSH (677 mg, 4.0 equiv) dropwise at 0° C. for 0.2 hours under N₂ atmosphere. 7-(5,6-dimethyl-1-(tetrahydro-2H-pyran-2-yl)-1H-indazol-4-yl)-2-(((2R,7aS)-2-fluorohexahydro-1H-pyrrolizin-7a-yl)methoxy)-4-methoxy-5,6,7,8-tetrahydropyrido[3,4-d]pyrimidine (1.5 g, 1.0 equiv) in DMAC (15 mL) was added below 15° C. The mixture was stirred at 60° C. for 1.2 hours. The reaction was quenched with saturated NH₄Cl aqueous (20 mL) at 0° C. and diluted with H₂O (500 mL). The mixture was extracted with ethyl acetate (5×30 mL) and the combined organic layers were dried over anhydrous Na₂SO₄, filtered and concentrated to afford the title compound (1.6 g, crude) as yellow foam; LCMS (ESI, M+1): m/z=537.4.

Step C. 7-(5,6-dimethyl-1-(tetrahydro-2H-pyran-2-yl)-1H-indazol-4-yl)-2-(((2R,7aS)-2-fluorohexahydro-1H-pyrrolizin-7a-yl)methoxy)-5,6,7,8-tetrahydropyrido[3,4-d] pyrimidin-4-yl 4-methylbenzenesulfonate: To a solution of 7-(5,6-dimethyl-1-(tetrahydro-2H-pyran-2-yl)-1H-indazol-4-yl)-2-(((2R,7aS)-2-fluorohexahydro-1H-pyrrolizin-7a-yl)methoxy)-5,6,7,8-tetrahydropyrido[3,4-d]pyrimidin-4-ol (1.6 g, 1.0 equiv) and N,N-diethylpropan-2-amine (1.16 g, 3.0 equiv) in THF (25 mL) were added DMAP (36.4 mg, 0.1 equiv) and TosCl (853 mg, 1.5 equiv) at 0° C. The mixture was stirred at 20° C. for 12 hours. The mixture was diluted with water (50 mL) and extracted with ethyl acetate (6×40 mL). The organic phase was dried over Na₂SO₄, concentrated, and purified by column chromatography [Al₂O₃, petroleum ether/ethyl acetate=10/1 to 1/2] to afford the title compound (1.53 g, 73% yield, over 2 steps) as yellow solid; LCMS (ESI, M+1): m/z=691.4.

Step D. 7-(5,6-dimethyl-1H-indazol-4-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate: To a solution of 7-(5,6-dimethyl-1-(tetrahydro-2H-pyran-2-yl)-1H-indazol-4-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (600 mg, 1.0 equiv) in DCM (3 mL) was added TFA (4.6 g, 47 equiv) at 0° C. The mixture was stirred at 20° C. for 0.5 hours. The mixture was added into saturated NaHCO₃ aqueous (40 mL) and DCM (20 mL) slowly at 0° C. (pH=8). The mixture was extracted with DCM (4×15 mL) at 0° C. The organic phase was dried over Na₂SO₄ and concentrated to afford the title compound (370 mg, 65% yield) as yellow foam; LCMS (ESI, M+1): m/z=607.4.

Step E. (R)-7-(7-(5,6-dimethyl-1H-indazol-4-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione: To a solution of 7-(5,6-dimethyl-1H-indazol-4-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (100 mg, 1.0 equiv) in DMF (0.5 mL) were added N,N-diethylpropan-2-amine (43 mg, 2.0 equiv) and (5R)-1,3,9-triazaspiro[4.5]decane-2,4-dione (41.8 mg, 1.5 equiv). The mixture was stirred at 40° C. for 12 hours. The reaction was filtered, purified by reversed phase flash chromatography {0.1% FA condition] and lyophilized to obtain the product. Then the product was purified again with prep-HPLC [column: Phenomenex luna C18 150×25 mm×10 m; mobile phase: [water (FA)-ACN]; B %: 12%-42%, 10 min] to afford the title compound (45.6 mg, 43% yield, 0.43 FA) as white solid (FA salt). 1H NMR (400 MHz, METHANOL-d₄) δ=8.48 (s, 1H), 8.02 (s, 1H), 7.19 (s, 1H), 5.59-5.27 (m, 1H), 4.41-4.25 (m, 2H), 4.19 (br s, 2H), 4.16 (br s, 1H), 4.04 (br d, J=12.4 Hz, 1H), 3.74-3.62 (m, 1H), 3.62-3.55 (m, 2H), 3.51 (br t, J=4.8 Hz, 2H), 3.41 (d, J=13.2 Hz, 1H), 3.28-3.16 (m, 2H), 2.90 (dt, J=1.6, 13.2 Hz, 2H), 2.62-2.43 (m, 1H), 2.41 (s, 4H), 2.36 (s, 3H), 2.30-2.21 (m, 1H), 2.21-2.02 (m, 4H), 1.99-1.80 (m, 3H); LCMS (ESI, M+1): m/z=604.4.

### Example 451

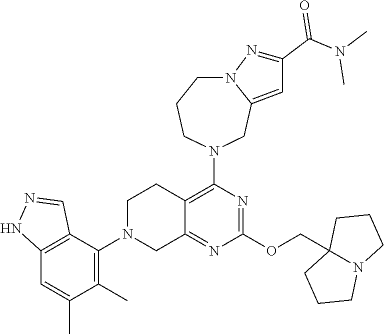 [SMILES: Cc1cc2[nH]ncc2c(N2CCc3c(nc(OCC45CCCN4CCC5)nc3N3CCCn4nc(C(=O)N(C)C)cc4C3)C2)c1C]

### 5-(7-(5,6-dimethyl-1H-indazol-4-yl)-2-((hexahydro-1H-pyrrolizin-7a-yl)methoxy)-5,6,7,8-tetrahydropyrido[3,4-d]pyrimidin-4-yl)-N,N-dimethyl-5, 6,7,8-tetrahydro-4H-pyrazolo[1,5-a][1,4]diazepine-2-carboxamide

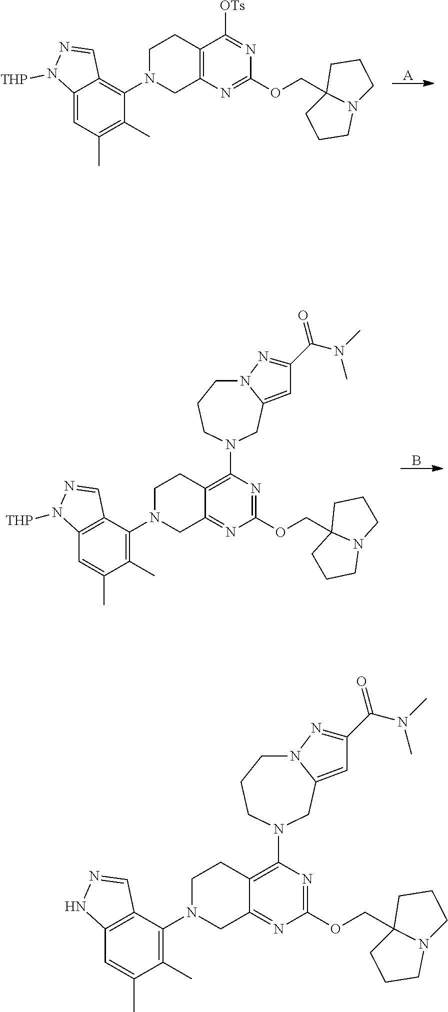

Step A. 5-(7-(5,6-dimethyl-1-(tetrahydro-2H-pyran-2-yl)-1H-indazol-4-yl)-2-((hexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide: To a solution of 7-(5,6-dimethyl-1-(tetrahydro-2H-pyran-2-yl)-1H-indazol-4-yl)-2-((hexahydro-1H-pyrrolizin-7a-yl)methoxy)-5,6,7,8-tetrahydropyrido[3,4-d]pyrimidin-4-yl 4-methylbenzenesulfonate (100 mg, 1.0 equiv) and N,N-dimethyl-5,6,7,8-tetrahydro-4H-pyrazolo[1,5-a][1,4]diazepine-2-carboxamide (79.5 mg, 2.6 equiv) in DMF (1 mL) were added N,N-diethylpropan-2-amine (153 mg, 8.0 equiv) and 4 Å molecular sieve (5.00 mg, 1.0 equiv). The reaction was stirred at 40° C. for 12 hours. The mixture was filtered and purified by reversed phase HPLC (0.1% FA condition) to afford the title compound (38.0 mg, 36% yield) as yellow solid.

Step B. 5-(7-(5,6-dimethyl-1H-indazol-4-yl)-2-((hexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide: A solution of 5-(7-(5,6-dimethyl-1-(tetrahydro-2H-pyran-2-yl)-1H-indazol-4-yl)-2-((hexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide (38.0 mg, 1.0 equiv) in HCl-MeOH (4M, 1.0 mL) was stirred at 20° C. for 1 hour. The mixture was concentrated under reduced pressure to remove solvent. The pH of residue was adjusted to 8 with Na₂CO₃ aqueous solution. The mixture was extracted with EtOAc (3×2 mL). The combined organic layers were washed with brine (2×3 mL), dried over anhydrous sodium sulfate, concentrated, and purified by prep-HPLC (column: Phenomenex luna C18 150×25 mm×10 μm; mobile phase: [water (FA) -ACN]; B %: 14%-44%, 10 min) to afford the title compound (10.5 g, 31% yield) as white solid (FA salt). ¹H NMR (400 MHz, METHANOL-d₄) δ=8.01 (s, 1H), 7.19 (s, 1H), 6.62 (s, 1H), 4.89 (s, 2H), 4.53-4.49 (m, 2H), 4.24 (s, 2H), 4.17 (br s, 2H), 4.09 (br t, J=5.2 Hz, 2H), 3.55-3.49 (m, 2H), 3.33 (br s, 3H), 3.07 (s, 3H), 3.01-2.86 (m, 4H), 2.41 (s, 3H), 2.36 (s, 3H), 2.22-1.82 (m, 12H); LCMS (ESI, M+1): m/z=625.6.

### Example 452

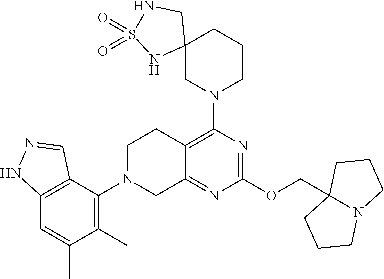 [SMILES: Cc1cc2[nH]ncc2c(N2CCc3c(nc(OCC45CCCN4CCC5)nc3N3CCCC4(CNS(=O)(=O)N4)C3)C2)c1C]

### 7-(7-(5,6-dimethyl-1H-indazol-4-yl)-2-((hexahydro-1H-pyrrolizin-7a-yl)methoxy)-5,6,7,8-tetrahydropyrido[3,4-d]pyrimidin-4-yl)-2-thia-1,3,7-triazaspiro[4.5]decane 2,2-dioxide

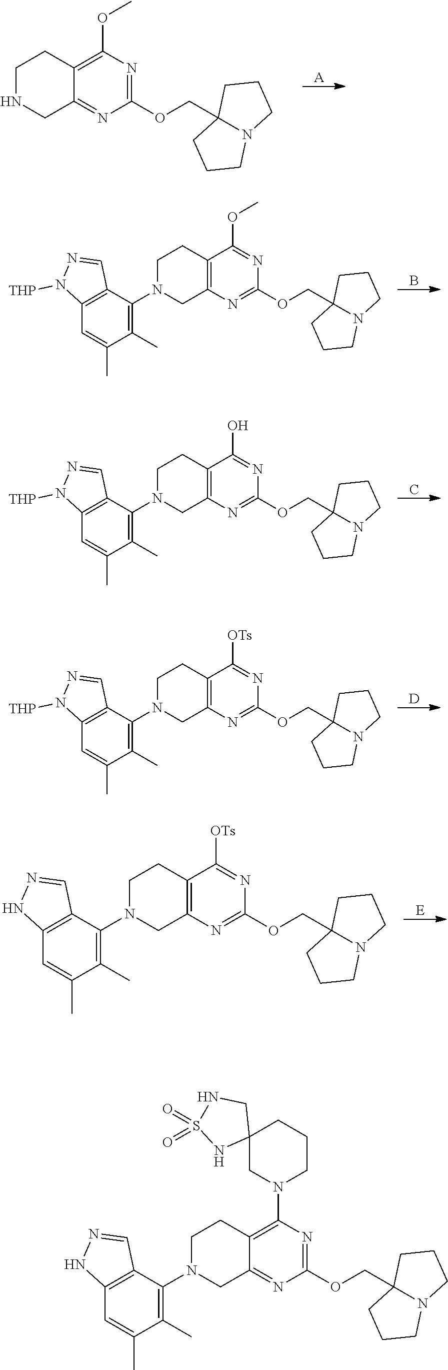

Step A. 7-(5,6-dimethyl-1-(tetrahydro-2H-pyran-2-yl)-1H-indazol-4-yl)-2-((hexahydro-1H-pyrrolizin-7a-yl)methoxy)-4-methoxy-5,6,7,8-tetrahydropyrido[3,4-d]pyrimidine: A mixture of 2-((hexahydro-1H-pyrrolizin-7a-yl)methoxy)-4-methoxy-5,6,7,8-tetrahydropyrido[3,4-d]pyrimidine (1.8 g, 1.0 equiv), 4-bromo-5,6-dimethyl-1-(tetrahydro-2H-pyran-2-yl)-1H-indazole (3.66 g, 2.0 equiv), Cs₂CO₃ (5.78 g, 3.0 equiv) and RuPhos (552 mg, 0.2 equiv) in toluene (25 mL) was degassed and purged with N₂ 3 times, Pd₂(dba)₃ (541 mg, 0.1 equiv) was added and the mixture was stirred at 110° C. for 12 hours under N₂ atmosphere. The reaction mixture was filtered through a pad of Celit. The filter cake was washed with ethyl acetate (50 mL). The filtrate was concentrated and purified with reversed phase flash [C18, 0.1% formic acid condition] to afford the title compound (1.2 g, 37% yield) as yellow solid; LCMS (ESI, M+1): m/z=533.3.

Step B. 7-(5,6-dimethyl-1-(tetrahydro-2H-pyran-2-yl)-1H-indazol-4-yl)-2-((hexahydro-1H-pyrrolizin-7a-yl)methoxy)-5,6,7,8-tetrahydropyrido[3,4-d]pyrimidin-4-ol: To a mixture of NaH (165 mg, 2 equiv) in DMAC (10 mL) was added EtSH (480 mg, 4.0 equiv) dropwise at 0° C. under N₂ atmosphere. The mixture was stirred at 5° C. for 0.1 hour. 7-(5,6-dimethyl-1-(tetrahydro-2H-pyran-2-yl)-1H-indazol-4-yl)-2-((hexahydro-1H-pyrrolizin-7a-yl)methoxy)-4-methoxy-5,6,7,8-tetrahydropyrido[3,4-d]pyrimidine (1.1 g, 1 equiv) in DMAC (10 mL) was added below 15 C. The mixture was stirred at 60° C. for 1.2 hours. The reaction mixture was quenched with saturated NH₄Cl aqueous (20 mL) at 0° C. and diluted with H₂O (500 mL). The mixture was extracted with ethyl acetate (5×30 mL). The combined organic layers were dried over anhydrous Na₂SO₄ and concentrated to afford the title compound (1 g, crude) as a red oil; LCMS (ESI, M+1): m/z=519.3.

Step C. 7-(5,6-dimethyl-1-(tetrahydro-2H-pyran-2-yl)-1H-indazol-4-yl)-2-((hexahydro-1H-pyrrolizin-7a-yl)methoxy)-5,6,7,8-tetrahydropyrido[3,4-d] pyrimidin-4-yl 4-methylbenzenesulfonate: To a solution of 7-(5,6-dimethyl-1-(tetrahydro-2H-pyran-2-yl)-1H-indazol-4-yl)-2-((hexahydro-1H-pyrrolizin-7a-yl)methoxy)-5,6,7,8-tetrahydropyrido[3,4-d]pyrimidin-4-ol (1 g, 1.0 equiv), DIPEA (748 mg, 3.0 equiv) and DMAP (23.6 mg, 0.1 equiv) in THF (10 mL) was added TsCl (551 mg, 1.5 equiv) at 0° C. The mixture was stirred at 25° C. for 3 hours. The reaction mixture was diluted with H₂O (50 mL) and extracted with DCM (3×10 mL). The combined organic phase was dried over anhydrous Na₂SO₄, concentrated and purified by column chromatography [Al₂O₃, petroleum ether/ethyl acetate=5/1 to 0/1 and ethyl acetate/MeOH=20/1] to afford the title compound (720 mg, 44% yield) as light-yellow solid; LCMS (ESI, M+1): m/z=673.3.

Step D. 7-(5,6-dimethyl-1H-indazol-4-yl)-2-((hexahydro-1H-pyrrolizin-7a-yl)methoxy)-5,6,7,8-tetrahydropyrido[3,4-d]pyrimidin-4-yl 4-methylbenzenesulfonate: To a solution of 7-(5,6-dimethyl-1-(tetrahydro-2H-pyran-2-yl)-1H-indazol-4-yl)-2-((hexahydro-1H-pyrrolizin-7a-yl)methoxy)-5,6,7,8-tetrahydropyrido[3,4-d]pyrimidin-4-yl 4-methylbenzenesulfonate (700 mg, 1 equiv) in DCM (6 mL) was added TFA (7 mL) at 0° C. The mixture was stirred at 25° C. for 2 hours. The reaction mixture was quenched with saturated NaHCO₃ aqueous (30 mL) at 0° C. The pH of the mixture was adjusted to 8 with NaHCO₃solid below 10° C. The mixture was extracted with DCM (4×10 mL). The combined organic layers were dried over anhydrous Na₂SO₄ and concentrated to compound (600 mg, crude) as yellow solid; LCMS (ESI, M+1): m/z=589.3.

Step E. 7-(7-(5,6-dimethyl-1H-indazol-4-yl)-2-((hexahydro-1H-pyrrolizin-7a-yl)methoxy)-5,6,7,8-tetrahydropyrido[3,4-d] pyrimidin-4-yl)-2-thia-1,3,7-triazaspiro[4.51decane 2,2-dioxide: To a solution of 7-(5,6-dimethyl-1H-indazol-4-yl)-2-((hexahydro-1H-pyrrolizin-7a-yl)methoxy)-5,6,7,8-tetrahydropyrido[3,4-d]pyrimidin-4-yl 4-methylbenzenesulfonate (160 mg, 1.0 equiv) and 2-thia-1,3,7-triazaspiro[4.5]decane 2,2-dioxide (90 mg, 1.7 equiv) in DMF (0.8 mL) were added DIPEA (70.2 mg, 2.0 equiv) and 4 Å molecular sieve (20 mg). The mixture was stirred at 40° C. for 14 hours under N₂ atmosphere. The mixture was filtered. The filtrate was purified with reversed phase flash chromatography [water (FA, 0.1%)/acetonitrile=7/3] and prep-HPLC [column: Waters Xbridge 150×25 mm×5 μm; mobile phase: water (NH₄HCO₃)-ACN]; B %: 36%-66%, 8 minutes] to afford the title compound (37.4 mg) as white solid; ¹H NMR (400 MHz, methanol-d₄) δ=8.05 (s, 1H), 7.17 (s, 1H), 4.16-4.12 (m, 4H), 3.98-3.88 (m, 1H), 3.80-3.62 (br s, 1H), 3.55-3.45 (m, 3H), 3.39 (d, J=12.0 Hz, 2H), 3.17 (d, J=12.0 Hz, 1H), 3.12-3.04 (m, 2H), 2.95-2.78 (m, 2H), 2.74-2.66 (m, 2H), 2.40 (s, 3H), 2.35 (s, 3H), 2.09-1.99 (m, 2H), 1.98-1.81 (m, 7H), 1.77-1.66 (m, 3H); LCMS (ESI, M+1): m/z=608.4.

### Example 453

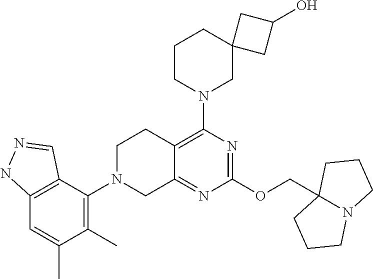 [SMILES: Cc1cc2[nH]ncc2c(N2CCc3c(nc(OCC45CCCN4CCC5)nc3N3CCCC4(CC(O)C4)C3)C2)c1C]

### 6-(7-(5,6-dimethyl-1H-indazol-4-yl)-2-((hexahydro-1H-pyrrolizin-7a-yl)methoxy)-5,6,7,8-tetrahydropyrido[3,4-d]pyrimidin-4-yl)-6-azaspiro[3.5]nonan-2-ol (isomer 1)

### Example 454

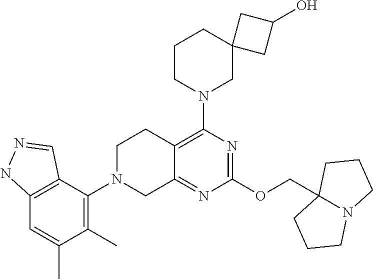 [SMILES: Cc1cc2[nH]ncc2c(N2CCc3c(nc(OCC45CCCN4CCC5)nc3N3CCCC4(CC(O)C4)C3)C2)c1C]

6-(7-(5,6-dimethyl-1H-indazol-4-yl)-2-((hexahydro-1H-pyrrolizin-7a-yl)methoxy)-5,6,7,8-tetrahydropyrido[3,4-d]pyrimidin-4-yl)-6-azaspiro[3.5]nonan-2-ol (isomer 2)

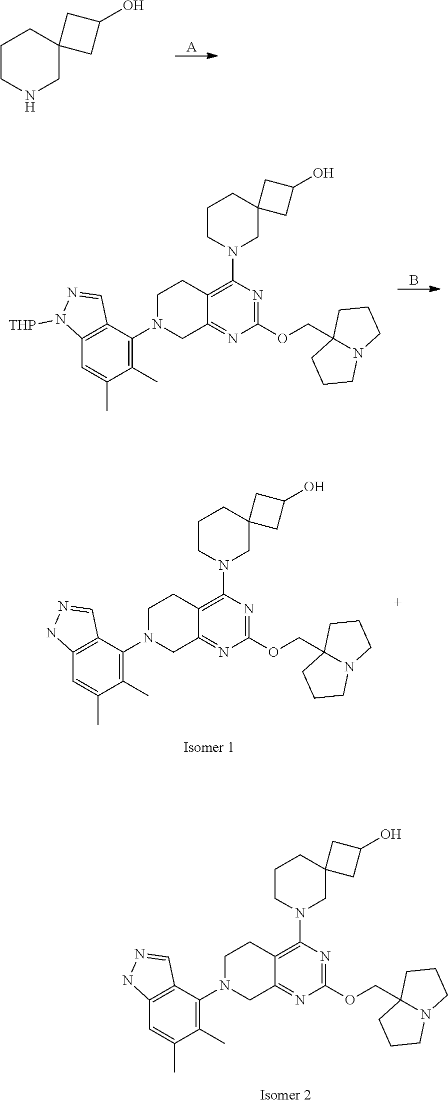

Step A. 6-(7-(5,6-dimethyl-1-(tetrahydro-2H-pyran-2-yl)-1H-indazol-4-yl)-2-((tetrahydro-1H-pyrrolizin-7a(5H)-yl)methoxy)-5,6,7,8-tetrahydropyrido[3,4-d]pyrimidin-4-yl)-6-azaspiro[3.5]nonan-2-ol: To a mixture of 7-(5,6-dimethyl-1-(tetrahydro-2H-pyran-2-yl)-1H-indazol-4-yl)-2-((hexahydro-1H-pyrrolizin-7a-yl)methoxy)-5,6,7,8-tetrahydropyrido[3,4-d]pyrimidin-4-yl 4-methylbenzenesulfonate (200 mg, 1.0 equiv) and 6-azaspiro[3.5]nonan-2-ol (100 mg, 1.9 equiv, HCl) in DMF (1 mL) were added N,N-diethylpropan-2-amine (576 mg, 15 equiv) and 4 Å molecular sieve (30 mg, 1.0 equiv). The reaction was stirred 40° C. for 12 hours. The mixture was filtered and purified by reversed phase flash chromatography [water (0.1%, FA)/acetonitrile] to afford the title compound (50 mg, 25% yield) as yellow solid. LCMS (ESI, M+1): m/z=642.7.

Step B. 6-(7-(5,6-dimethyl-1H-indazol-4-yl)-2-((hexahydro-1H-pyrrolizin-7a-yl)methoxy)-5,6,7,8-tetrahydropyrido[3,4-d]pyrimidin-4-yl)-6-azaspiro[3.5]nonan-2-ol: A mixture of 6-(7-(5,6-dimethyl-1-(tetrahydro-2H-pyran-2-yl)-1H-indazol-4-yl)-2-((tetrahydro-1H-pyrrolizin-7a(5H)-yl)methoxy)-5,6,7,8-tetrahydropyrido[3,4-d]pyrimidin-4-yl)-6-azaspiro[3.5]nonan-2-ol (110 mg, 1.0 equiv) in HCl/MeOH (4 M, 1.0 equiv) was stirred at 25° C. for 0.5 hours. The mixture was concentrated under vacuum. The pH value of the residue was adjusted to 8 with NaHCO₃ solution. The mixture was extracted with ethyl acetate (2×5 mL). The combined organic layers were washed with brine (5 mL), dried over anhydrous sodium sulfate, concentrated, and purified with prep-HPLC (column: Phenomenex luna C18 150*25 mm*10 μm; mobile phase: [water (FA)-ACN];B %: 16%-46%,10 min. to afford the title compounds.

The first peak as Example 453 (10.5 mg yellow solid, FA salt). ¹H NMR (400 MHz, METHANOL-d₄) δ=8.21-7.95 (m, 1H), 7.19 (s, 1H), 4.45-4.29 (m, 2H), 4.28-4.07 (m, 3H), 3.60-3.46 (m, 5H), 3.45-3.33 (m, 3H), 3.05-2.95 (m, 2H), 2.92-2.77 (m, 1H), 2.41 (s, 3H), 2.27-2.12 (m, 4H), 2.10-1.97 (m, 4H), 1.96-1.82 (m, 2H), 1.81-1.53 (m, 6H) LCMS (ESI, M+1): m/z=558.2.

The second peak as Example 454 (7.80 mg yellow solid, FA salt). ¹H NMR (400 MHz, METHANOL-d₄) δ=8.55 (s, 1H), 8.03 (s, 1H), 7.19 (s, 1H), 4.33 (s, 2H), 4.27-4.16 (m, 3H), 3.58-3.47 (m, 5H), 3.44-3.35 (m, 3H), 3.06-2.96 (m, 2H), 2.93-2.71 (m, 2H), 2.41 (s, 3H), 2.37 (s, 3H), 2.22-2.13 (m, 4H), 2.12-1.99 (m, 4H), 1.88 (br s, 2H), 1.79-1.65 (m, 6H) LCMS (ESI, M+1): m/z=558.4.

### Example 455

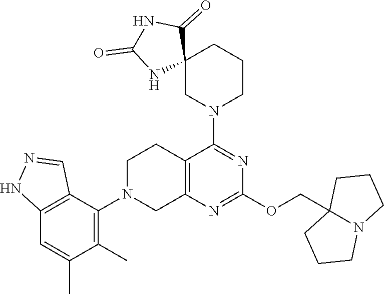 [SMILES: Cc1cc2[nH]ncc2c(N2CCc3c(nc(OCC45CCCN4CCC5)nc3N3CCC[C@]4(C3)NC(=O)NC4=O)C2)c1C]

### (R)-7-(7-(5,6-dimethyl-1H-indazol-4-yl)-2-((hexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione

Synthesized according to Example 452. The title compound was obtained white solid. ¹H NMR (400 MHz, METHANOL-d₄) δ=8.05 (s, 1H), 7.18 (s, 1H), 4.23-4.04 (m, 6H), 3.50 (br d, J=4.8 Hz, 2H), 3.38 (br d, J=13.2 Hz, 1H), 3.16-3.02 (m, 3H), 2.89 (br dd, J=3.6, 7.2 Hz, 2H), 2.76-2.67 (m, 2H), 2.41 (s, 3H), 2.36 (s, 3H), 2.16-1.98 (m, 4H), 1.94-1.83 (m, 6H), 1.79-1.65 (m, 2H); LCMS (ESI, M+1): m/z=586.3.

### Example 456

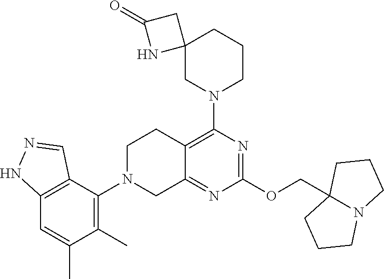 [SMILES: Cc1cc2[nH]ncc2c(N2CCc3c(nc(OCC45CCCN4CCC5)nc3N3CCCC4(CC(=O)N4)C3)C2)c1C]

### 6-(7-(5,6-dimethyl-1H-indazol-4-yl)-2-((hexahydro-1H-pyrrolizin-7a-yl)methoxy)-5,6,7,8-tetrahydropyrido[3,4-d]pyrimidin-4-yl)-1,6-diazaspiro[3.5]nonan-2-one

Synthesized according to Example 452. The title compound was obtained as white solid (FA salt). ¹H NMR (400 MHz, METHANOL-d₄) δ=8.02 (s, 1H), 7.18 (s, 1H), 4.37-4.28 (m, 2H), 4.22-4.16 (br s, 2H), 3.82 (br d, J=13.2 Hz, 1H), 3.77-3.63 (m, 1H), 3.58 (br d, J=13.2 Hz, 1H), 3.50 (br t, J=4.4 Hz, 2H), 3.46-3.33 (m, 3H), 3.05-2.95 (m, 2H), 2.92-2.61 (m, 4H), 2.45-2.30 (m, 6H), 2.16 (br dd, J=6.4, 12.4 Hz, 2H), 2.11-1.93 (m, 6H), 1.92-1.78 (m, 4H); LCMS (ESI, M+1): m/z=557.3.

### Example 457

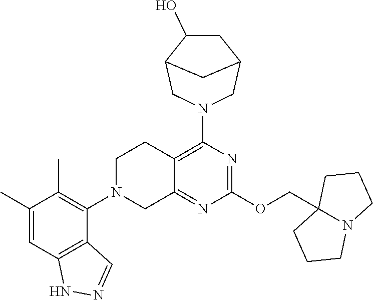 [SMILES: Cc1cc2[nH]ncc2c(N2CCc3c(nc(OCC45CCCN4CCC5)nc3N3CC4CC(O)C(C4)C3)C2)c1C]

### 3-(7-(5,6-dimethyl-1H-indazol-4-yl)-2-((hexahydro-1H-pyrrolizin-7a-yl)methoxy)-5,6,7,8-tetrahydropyrido[3,4-d]pyrimidin-4-yl)-3-azabicyclo[3.2.1]octan-6-ol

Synthesized according to Example 452. The title compound was obtained as white solid (FA salt). ¹H NMR (400 MHz, METHANOL-d₄) δ=8.03 (s, 1H), 7.18 (s, 1H), 4.61-4.21 (m, 5H), 4.17 (s, 2H), 3.52-3.38 (m, 4H), 3.21 (br d, J=10.8 Hz, 2H), 3.11-2.94 (m, 3H), 2.91-2.75 (m, 1H), 2.40 (s, 3H), 2.35 (s, 3H), 2.30-2.16 (m, 5H), 2.14-1.99 (m, 4H), 1.98-1.90 (m, 2H), 1.74 (q, J=12.0 Hz, 2H), 1.42 (br d, J=7.2 Hz, 1H); LCMS (ESI, M+1): m/z=544.4.

### Example 458

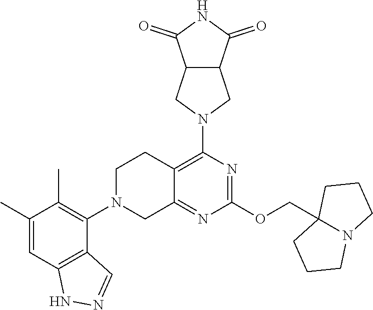 [SMILES: Cc1cc2[nH]ncc2c(N2CCc3c(nc(OCC45CCCN4CCC5)nc3N3CC4C(=O)NC(=O)C4C3)C2)c1C]

### 5-(7-(5,6-dimethyl-1H-indazol-4-yl)-2-((hexahydro-1H-pyrrolizin-7a-yl)methoxy)-5,6,7,8-tetrahydropyrido[3,4-d]pyrimidin-4-yl)tetrahydropyrrolo[3,4-c]pyrrole-1,3(2H,3aH)-dione

Synthesized according to Example 452. The title compound was obtained as white solid (FA salt). ¹H NMR (400 MHz, DMSO+D₂O) δ=8.04 (s, 1H), 7.16 (s, 1H), 4.19 (br s, 4H), 4.05 (br s, 2H), 3.57 (br d, J=1.6 Hz, 2H), 3.49-3.38 (m, 4H), 3.3-3.20 (m, 2H), 2.97-2.87 (m, 2H), 2.82-2.62 (m, 2H), 2.32 (s, 3H), 2.23 (s, 3H), 1.98 (br s, 8H; LCMS (ESI, M+1): m/z=557.4.

### Example 459

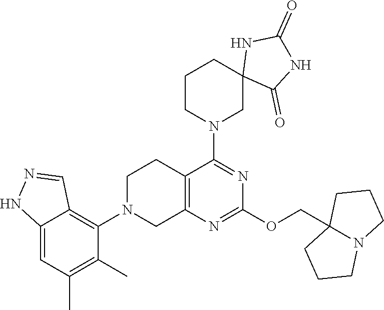 [SMILES: Cc1cc2[nH]ncc2c(N2CCc3c(nc(OCC45CCCN4CCC5)nc3N3CCCC4(C3)NC(=O)NC4=O)C2)c1C]

### 7-(7-(5,6-dimethyl-1H-indazol-4-yl)-2-((hexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione

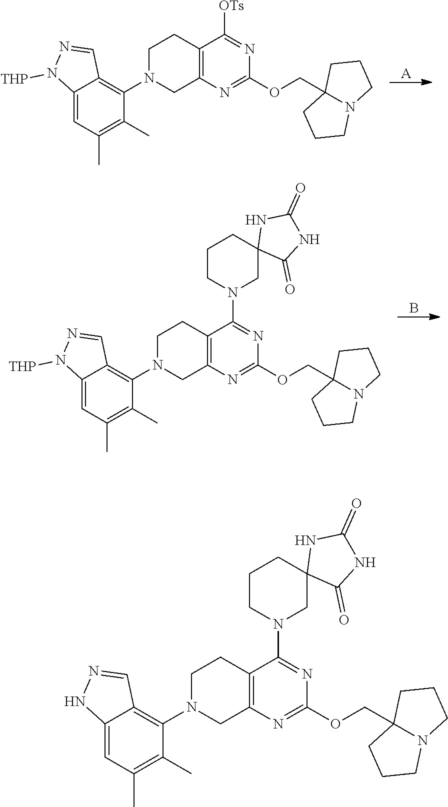

Synthesized according to Example 451. The title compound was obtained as yellow solid. ¹H NMR (400 MHz, METHANOL-d₄) δ=8.04 (s, 1H), 7.17 (s, 1H), 4.22-4.01 (m, 6H), 3.50 (br t, J=4.8 Hz, 2H), 3.37-3.32 (m, 2H), 3.08-3.02 (m, 2H), 2.97-2.77 (m, 2H), 2.67 (td, J=6.4, 10.4 Hz, 2H), 2.40 (s, 3H), 2.35 (s, 3H), 2.11-1.98 (m, 3H), 1.96-1.77 (m, 7H), 1.75-1.62 (m, 2H). LCMS (ESI, M+1): m/z=586.3.

### Example 460

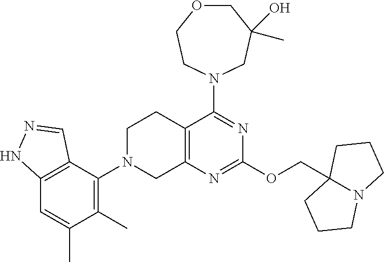 [SMILES: Cc1cc2[nH]ncc2c(N2CCc3c(nc(OCC45CCCN4CCC5)nc3N3CCOCC(C)(O)C3)C2)c1C]

### 4-(7-(5,6-dimethyl-1H-indazol-4-yl)-2-((hexahydro-1H-pyrrolizin-7a-yl)methoxy)-5,6,7,8-tetrahydropyrido[3,4-d]pyrimidin-4-yl)-6-methyl-1,4-oxazepan-6-ol

Synthesized according to Example 452. The title compound was obtained as white solid; ¹H NMR (400 MHz, METHANOL-d₄) δ=8.05 (br s, 1H), 7.17 (s, 1H), 4.18 (br s, 2H), 4.13-3.99 (m, 4H), 3.96-3.84 (m, 2H), 3.70-3.57 (m, 3H), 3.56-3.44 (m, 3H), 3.16-3.01 (m, 2H), 2.98-2.77 (m, 2H), 2.71 (td, J=6.4, 10.4 Hz, 2H), 2.40 (s, 3H), 2.35 (s, 3H), 2.07-1.97 (m, 2H), 1.95-1.81 (m, 4H), 1.77-1.67 (m, 2H), 1.19 (s, 3H); LCMS (ESI, M+1): m/z=548.3.

### Example 461

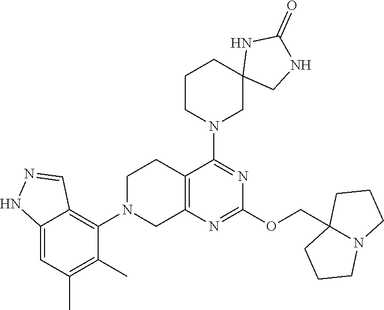 [SMILES: Cc1cc2[nH]ncc2c(N2CCc3c(nc(OCC45CCCN4CCC5)nc3N3CCCC4(CNC(=O)N4)C3)C2)c1C]

### 7-(7-(5,6-dimethyl-1H-indazol-4-yl)-2-((hexahydro-1H-pyrrolizin-7a-yl)methoxy)-5,6,7,8-tetrahydropyrido[3,4-d]pyrimidin-4-yl)-1,3,7-triazaspiro[4.5]decan-2-one

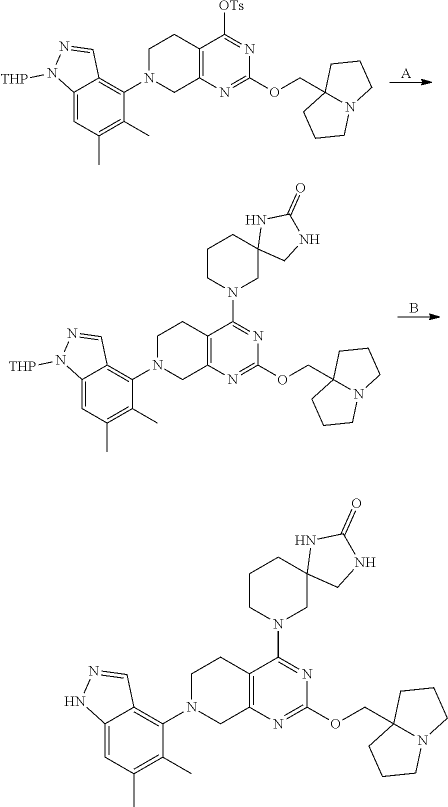

Synthesized according to Example 451. The title compound was obtained as white solid (FA salt). ¹H NMR (400 MHz, METHANOL-d₄) δ=8.02 (s, 1H), 7.19 (s, 1H), 4.50-4.35 (m, 2H), 4.21 (br s, 2H), 3.71-3.47 (m, 8H), 3.38 (br d, J=9.2 Hz, 1H), 3.25 (d, J=9.6 Hz, 1H), 3.22-3.12 (m, 2H), 3.04-2.71 (m, 2H), 2.41 (s, 3H), 2.36 (s, 3H), 2.30-2.21 (m, 2H), 2.20-2.07 (m, 4H), 2.06-1.96 (m, 2H), 1.94-1.68 (m, 4H). LCMS (ESI, M+1): m/z=572.6.

### Example 462

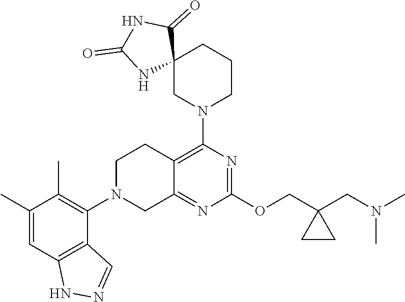

### (R)-7-(7-(5,6-dimethyl-1H-indazol-4-yl)-2-((1-((dimethylamino)methyl)cyclopropyl)methoxy)-5,6,7,8-tetrahydropyrido[3,4-d]pyrimidin-4-yl)-1,3,7-triazaspiro[4.5]decane-2,4-dione

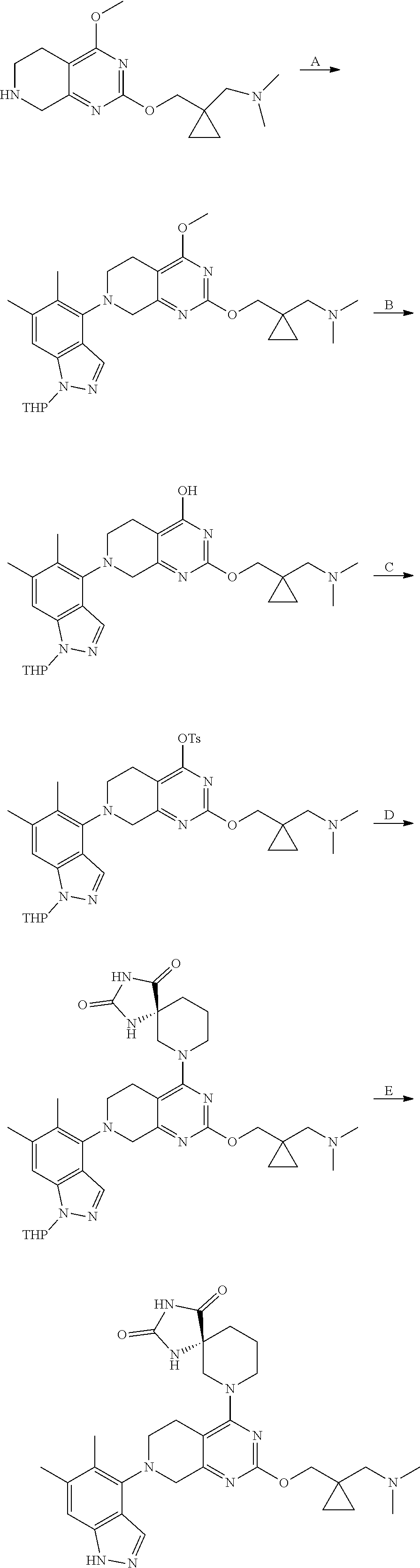

Step A. 1-(1-(((7-(5,6-dimethyl-1-(tetrahydro-2H-pyran-2-yl)-1H-indazol-4-yl-4-methoxy-5,6,7.8-tetrahydropyrido[3,4-d] pyrimidin-2-yloxy)methyllcyclopropyll-N,N-dimethylmethanamine: A mixture of 1-(1-(((4-methoxy-5,6,7,8-tetrahydropyrido[3,4-d]pyrimidin-2-yl)oxy)methyl)cyclopropyl)-N,N-dimethylmethanamine (500 mg, 1.0 equiv), 4-bromo-5,6-dimethyl-1-(tetrahydro-2H-pyran-2-yl)-1H-indazole (528 mg, 1.0 equiv), Cs₂CO₃ (1.67 g, 3.0 equiv), and Xantphos Pd G4 (164 mg, 0.1 equiv) in dioxane (20 mL) was degassed and purged with N₂ 3 times, and then the mixture was stirred at 90° C. for 12 hours under N₂ atmosphere. The reaction was filtered and washed with ethyl acetate (30 mL). The filtrate was concentrated under reduced pressure to dryness. The mixture was purified by column chromatography (SiO₂, petroleum ether/ethyl acetate=1/1 to dichloromethane/methanol=10/1). The crude product was purified by reversed-phase flash (0.1% FA condition) to afford the title compound (500 mg, 23% yield) as a white solid; LCMS (ESI, M+1): m/z=521.3.

Step B. 7-(5,6-dimethyl-1-(tetrahydro-2H-pyran-2-yl)-1H-indazol-4-yll-2-((1-((dimethylamino)methyllevelopropylmethoxy)-5,6,7,8-tetrahydropyrido[3,4-d] pyrimidin-4-ol: To a solution of EtSH (262 mg, 4.0 equiv) in DMAc (5 mL) was added NaH (84.5 mg, 60% purity, 2.0 equiv) slowly at 0° C. under N₂ atmosphere. The mixture was stirred at 25° C. for 30 minutes, and then 1-(1-(((7-(5, 6-dimethyl-1-(tetrahydro-2H-pyran-2-yl)-1H-indazol-4-yl)-4-methoxy-5,6,7,8-tetrahydropyrido[3,4-d]pyrimidin-2-yl)oxy)methyl)cyclopropyl)-N,N-dimethylmethanamine (550 mg, 1.0 equiv) in DMAc (5 mL) was added dropwise at 25° C. The resulting mixture was stirred at 60° C. for 1 hour. The mixture was quenched with water (10 mL) slowly at 0° C. for 5 minutes. The crude product was purified by reversed-phase flash (C18, 0.1% FA condition) to afford the title compound (250 mg, 46% yield) as a yellow solid; LCMS (ESI, M+1): m/z=507.2.

Step C. 7-(5,6-dimethyl-1-(tetrahydro-2H-pyran-2-yl)-1H-indazol-4-yl)-2-((1-((dimethylamino)methyl)cyclopropyl)methoxy)-5,6,7,8-tetrahydropyrido[3,4-d] pyrimidin-4-yl 4-methylbenzenesulfonate: To a solution of 7-(5,6-dimethyl-1-(tetrahydro-2H-pyran-2-yl)-1H-indazol-4-yl)-2-((1-((dimethylamino)methyl)cyclopropyl)methoxy)-5,6,7,8-tetrahydropyrido[3,4-d]pyrimidin-4-ol (150 mg, 1.0 equiv) in DCM (3 mL) were added N,N-diethylpropan-2-amine (114 mg, 3.0 equiv) and DMAP (3.62 mg, 0.1 equiv). The mixture was stirred at 0° C. for 10 minutes, and then 4-methylbenzenesulfonyl chloride (84.7 mg, 1.5 equiv) in DCM (3 mL) was added dropwise at 0° C. The mixture was stirred at 25° C. for 2 hours. The mixture was quenched with water (10 mL) and extracted with ethyl acetate (2×8 mL). The combined organic layers were dried over anhydrous sodium sulfate, concentrated, and purified by reversed-phase flash (0.1% FA condition) to afford the title compound (120 mg, 52% yield) as a white solid; LCMS (ESI, M+1): m/z=661.1

Step D. (5R)-7-(7-(5,6-dimethyl-1-(tetrahydro-2H-pyran-2-yl)-1H-indazol-4-yl)-2-((1-((dimethylamino)methyl)cyclopropyl)methoxy)-5,6,7,8-tetrahydropyrido[3,4-d] pyrimidin-4-yl)-1,3,7-triazaspiro[4.5]decane-2,4-dione: To a solution of 7-(5,6-dimethyl-1-(tetrahydro-2H-pyran-2-yl)-1H-indazol-4-yl)-2-((1-((dimethylamino)methyl)cyclopropyl)methoxy)-5,6,7,8-tetrahydropyrido[3,4-d]pyrimidin-4-yl 4-methylbenzenesulfonate (100 mg, 1.0 equiv) and (R)-1,3,7-triazaspiro[4.5]decane-2,4-dione (38.4 mg, 1.5 equiv) in DMF (0.3 mL) were added N,N-diethylpropan-2-amine (19.6 mg, 1.0 equiv) and 4 Å MS (25.0 mg). The mixture was stirred at 40° C. for 12 hours. The mixture was quenched with water (10 mL) and extracted with ethyl acetate (2×8 mL). The combined organic layers were dried over anhydrous sodium sulfate, concentrated, purified by prep-HPLC (column: Waters Xbridge 150×25 mm×5 μm; A: water (NH₄HCO₃); B: ACN, B %: 48%-78%, 8 min) and lyophilized to afford the title compound (20.0 mg, 17% yield) as a white solid; LCMS (ESI, M+1): m/z=658.5.

Step E. (R)-7-(7-(5,6-dimethyl-1H-indazol-4-yl)-2-((1-((dimethylamino)methyl)cyclopropyl)methoxy)-5,6,7,8-tetrahydropyrido[3,4-d]pyrimidin-4-yl)-1,3,7-triazaspiro[4.5]decane-2,4-dione: To a solution of (5R)-7-(7-(5,6-dimethyl-1-(tetrahydro-2H-pyran-2-yl)-1H-indazol-4-yl)-2-((1-((dimethylamino)methyl)cyclopropyl)methoxy)-5,6,7,8-tetrahydropyrido[3,4-d]pyrimidin-4-yl)-1,3,7-triazaspiro[4.5]decane-2,4-dione (30.0 mg, 1.0 equiv) in DCM (0.1 mL) was added TFA (1.39 g, 266 equiv). The mixture was stirred at 0° C. for 0.5 hours. The mixture was quenched with water (3 mL) and extracted with ethyl acetate (3×2 mL). The combined organic layers were washed with brine (3×2 mL), dried over anhydrous sodium sulfate, concentrated, purified by prep-HPLC [column: Phenomenex luna C18 150 x 25 mm×10 μm; A: water (FA); B: ACN, B %: 12%-32%, over 2 min] and lyophilized to afford the title compound (7.97 mg, 29% yield) as a yellow solid (FA salt). 1H NMR (400 MHz, METHANOL-d₄) δ=8.72-8.40 (m, 1H), 8.02 (s, 1H), 7.20 (s, 1H), 4.27 (s, 2H), 4.24-4.20 (m, 2H), 4.16-4.12 (m, 1H), 4.06-3.98 (m, 1H), 3.52-3.48 (m, 2H), 3.40 (br d, J=13.2 Hz, 2H), 3.20-3.12 (m, 2H), 2.88 (s, 8H), 2.42 (s, 3H), 2.36 (s, 3H), 2.16-2.04 (m, 1H), 1.96-1.84 (m, 3H), 0.91 (br s, 2H), 0.84-0.72 (m, 2H); LCMS (ESI, M+1): m/z=574.2.

### Example 463

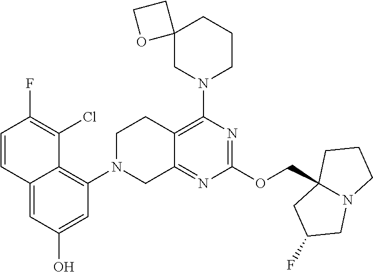 [SMILES: Oc1cc(N2CCc3c(nc(OC[C@@]45CCCN4C[C@H](F)C5)nc3N3CCCC4(CCO4)C3)C2)c2c(Cl)c(F)ccc2c1]

### 5-chloro-6-fluoro-4-(2-(((2R,7aS)-2-fluorohexahydro-1H-pyrrolizin-7a-yl)methoxy)-4-(1-oxa-6-azaspiro[3.5]nonan-6-yl)-5,6-dihydropyrido[3,4-d]pyrimidin-7(8H)-yl)naphthalen-2-ol

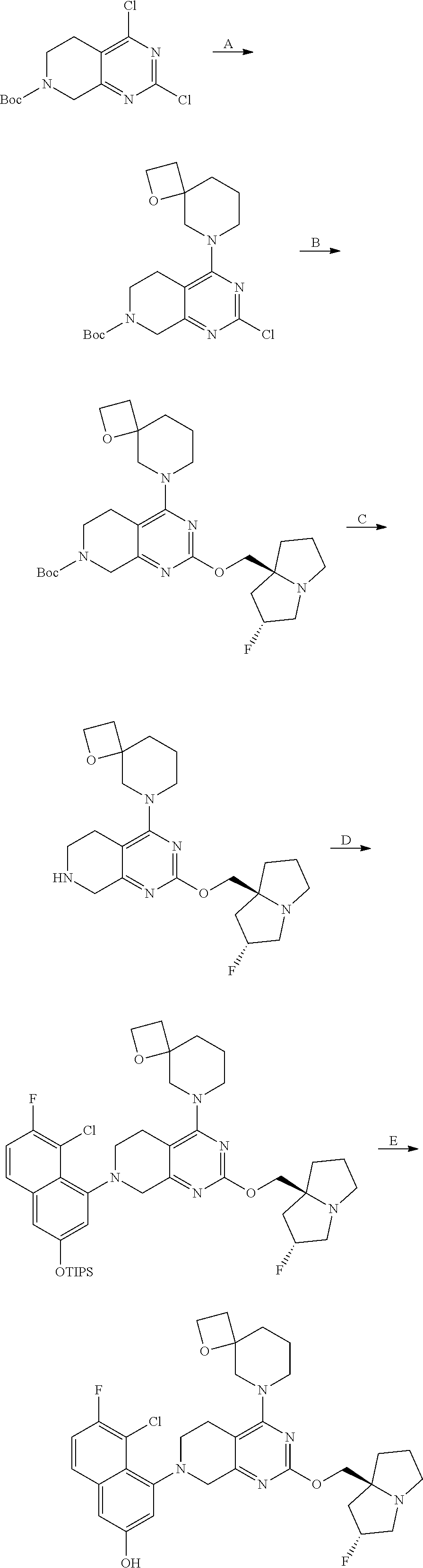

Step A. tert-butyl 2-chloro-4-(1-oxa-6-azaspiro[3.5]nonan-6-yl)-5,6-dihydropyrido[3,4-d]pyrimidine-7(8H)-carboxylate: To a solution of tert-butyl 2,4-dichloro-5,6-dihydropyrido[3,4-d]pyrimidine-7(8H)-carboxylate (1 g, 1.0 equiv) in DCM (10 mL) were added 1-oxa-8-azaspiro[3.5]nonane (714.1 mg, 1.0 equiv) and N,N-diethylpropan-2-amine (2.1 g, 5.0 equiv). The mixture was stirred at 20° C. for 2 hours. The reaction was diluted with water (20 mL) and extracted with DCM (20 mL×3). The combined organic layers were dried, filtered, concentrated, and purified by flash silica gel chromatography (ISCO®; 12 g SepaFlash® Silica Flash Column, Eluent of 10-50% Ethyl acetate/Petroleum ether gradient @50 mL/min) to afford the title compound (1.1 g, 82.2% yield) as yellow solid. ¹H NMR (400 MHz, CiLOROFORM-d) δ=4.61-4.44 (m, 4H), 3.85 (br d, J=13.2 Hz, 1H), 3.67-3.48 (m, 3H), 3.41 (br d, J=13.2 Hz, 1H), 3.13 (br t, J=10.0 Hz, 1H), 2.85-2.73 (m, 1H), 2.70-2.60 (m, 1H), 2.45-2.35 (m, 2H), 2.17-2.07 (m, 1H), 1.94-1.75 (m, 2H), 1.70-1.61 (m, 1H), 1.49 (s, 9H); LCMS (ESI, M+1): m/z=395.3.

Step B. tert-butyl 2-(((2R,7aS)-2-fluorohexahydro-1H-pyrrolizin-7a-yl)methoxy)-4-(1-oxa-6-azaspiro[3.5]nonan-6-yl)-5,6-dihydropyrido[3,4-d]pyrimidine-7(8H)-carboxylate: A mixture of tert-butyl 2-chloro-4-(1-oxa-6-azaspiro[3.5]nonan-6-yl)-5,6-dihydropyrido[3,4-d]pyrimidine-7(8H)-carboxylate (1.1 g, 1.0 equiv), ((2R,7aS)-2-fluorotetrahydro-1H-pyrrolizin-7a(5H)-yl)methanol (443 mg, 1.0 equiv), BINAP (347 mg, 0.2 equiv), Pd(OAc)₂ (62.5 mg, 0.1 equiv), and Cs₂CO₃ (2.7 g, 3.0 equiv) in toluene (11 mL) was degassed and purged with N₂ 3 times, and then the mixture was stirred at 110° C. for 12 hours under N₂ atmosphere. The reaction was filtered and the filtrate was diluted with water (40 mL) and extracted with EtOAc (30 mL×3). The combined organic layers were dried, filtered, concentrated, and purified by reversed phase HPLC [C8, 0.1% formic acid condition] to afford the title compound (950 mg, 65.9% yield) as green solid. ¹H NMR (400 MHz, CHLOROFORM-d) δ=5.37-5.16 (m, 1H), 4.54 (t, J=8.0 Hz, 2H), 4.45 (s, 2H), 3.99 (br d, J=9.2 Hz, 1H), 3.68 (br dd, J=2.8, 13.2 Hz, 1H), 3.54 (br s, 2H), 3.44 (br d, J=13.2 Hz, 2H), 3.31-3.21 (m, 2H), 3.15 (br d, J=6.8 Hz, 2H), 3.03-2.90 (m, 1H), 2.77-2.58 (m, 2H), 2.43-2.34 (m, 2H), 2.29-2.10 (m, 3H), 2.03 (br d, J=9.6 Hz, 1H), 1.98-1.79 (m, 5H), 1.62 (br s, 2H), 1.49 (s, 9H).

Step C. 6-(2-(((2R,7aS)-2-fluorohexahydro-1H-pyrrolizin-7a-yl)methoxy)-5,6,7,8-tetrahydropyrido[3,4-d] pyrimidin-4-yl)-1-oxa-6-azaspiro[3.5]nonane: To a solution of tert-butyl 2-(((2R,7aS)-2-fluorohexahydro-1H-pyrrolizin-7a-yl)methoxy)-4-(1-oxa-6-azaspiro[3.5]nonan-6-yl)-5,6-dihydropyrido[3,4-d]pyrimidine-7(8H)-carboxylate (300 mg, 1.0 equiv) in DCM (3.0 mL) was added TFA (2.3 g, 35.0 equiv). The mixture was stirred at 20° C. for 1 hour. The reaction was neutralized with aqueous NaOH (4 M) until pH=7, then was extracted with DCM (25 mL×3). The combined organic layers were dried, filtered, concentrated, and purified by prep-HPLC [C8, 0.1% NH₃—H₂O condition] to afford the title compound (196 mg, 79.4% yield) as brown oil. ¹H NMR (400 MHz, METHANOL-d₄) δ=5.40-5.17 (m, 1H), 4.58-4.47 (m, 1H), 4.20-4.11 (m, 1H), 4.09-4.02 (m, 1H), 4.00 (br s, 1H), 3.85-3.78 (m, 2H), 3.75-3.50 (m, 3H), 3.27-3.19 (m, 2H), 3.17 (s, 1H), 3.05-2.88 (m, 3H), 2.80-2.59 (m, 2H), 2.42 (t, J=8.0 Hz, 1H), 2.34-2.21 (m, 2H), 2.21-2.05 (m, 3H), 2.05-1.57 (m, 6H); LCMS (ESI, M+1): m/z=418.1.

Step D. 6-(7-(8-chloro-7-fluoro-3-((triisopropylsilyl)oxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-1-oxa-6-azaspiro[3.5]nonane: A mixture of 6-(2-(((2R,7aS)-2-fluorohexahydro-1H-pyrrolizin-7a-yl)methoxy)-5,6,7,8-tetrahydropyrido[3,4-d]pyrimidin-4-yl)-1-oxa-6-azaspiro[3.5]nonane (90 mg, 1.0 equiv), (8-chloro-7-fluoro-3-triisopropylsilyloxy-1-naphthyl) trifluoromethanesulfonate (129 mg, 1.2 equiv), Pd₂(dba)₃ (19.7 mg, 0.1 equiv), Cs₂CO₃ (210 mg, 3.0 equiv), and Xantphos (25.0 mg, 0.2 equiv) in toluene (1 mL) was degassed and purged with N₂ 3 times, and then the mixture was stirred at 110° C. for 12 hours under N₂ atmosphere. The reaction was extracted, dried, filtered, and purified by prep-TLC (SiO2, DCM: MeOH=10:1) to afford the title compound (17 mg, 7.2% yield) as a brown solid. LCMS (ESI, M+1): m/z=768.3.

Step E. 5-chloro-6-fluoro-4-(2-(((2R,7aS)-2-fluorohexahydro-1H-pyrrolizin-7a-yl)methoxy)-4-(1-oxa-6-azaspiro[3.5]nonan-6-yl)-5,6-dihydropyrido[3,4-d]pyrimidin-7(8H)-yl)naphthalen-2-ol: To a solution of 6-(7-(8-chloro-7-fluoro-3-((triisopropylsilyl)oxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1-oxa-6-azaspiro[3.5]nonane (17 mg, 1.0 equiv) in DMF (1 mL) was added CsF (50.4 mg, 15.0 equiv). The mixture was stirred at 20° C. for 1 hour. The reaction was extracted, filtered, concentrated, and purified by prep-HPLC [C18, 0.1% formic acid condition] to afford the title compound (5.5 mg) as white solid. ¹H NMR (400 MHz, METHANOL-d₄) δ=7.62 (dd, J=5.6, 9.2 Hz, 1H), 7.27 (t, J=8.8 Hz, 1H), 6.95 (s, 2H), 5.46-5.17 (m, 1H), 4.59-4.50 (m, 2H), 4.31-4.09 (m, 4H), 4.00-3.83 (m, 1H), 3.74-3.63 (m, 2H), 3.56-3.45 (m, 2H), 3.25 (br s, 2H), 3.18-3.00 (m, 3H), 2.79-2.59 (m, 1H), 2.55-2.31 (m, 3H), 2.28-2.21 (m, 1H), 2.20-2.09 (m, 2H), 2.04-1.79 (m, 6H), 1.76-1.57 (m, 1H); LCMS (ESI, M+1): m/z=612.3.

### Example 464

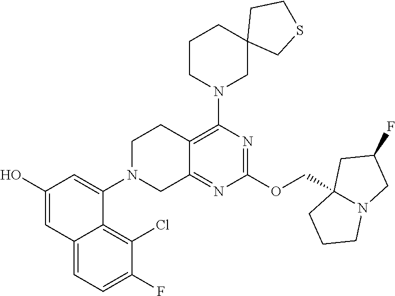 [SMILES: Oc1cc(N2CCc3c(nc(OC[C@@]45CCCN4C[C@H](F)C5)nc3N3CCCC4(CCSC4)C3)C2)c2c(Cl)c(F)ccc2c1]

### 5-chloro-6-fluoro-4-(2-(((2R,7aS)-2-fluorohexahydro-1H-pyrrolizin-7a-yl)methoxy)-4-(2-thia-7-azaspiro[4.5]decan-7-yl)-5,6-dihydropyrido[3,4-d]pyrimidin-7(8H)-yl)naphthalen-2-ol

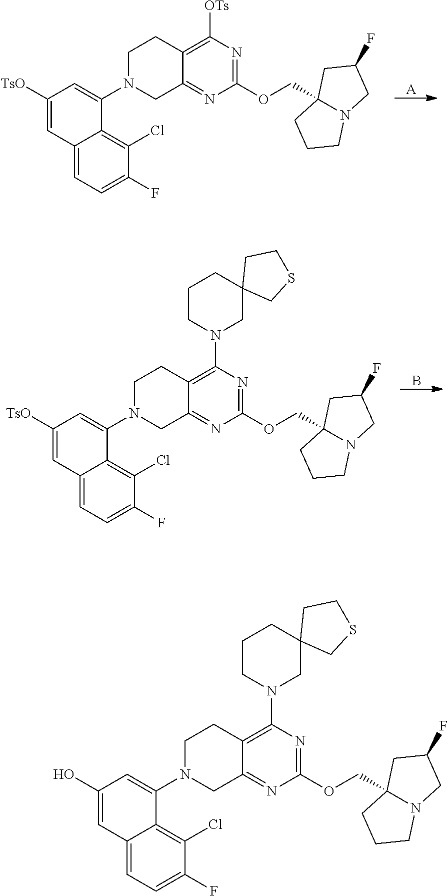

Step A. 5-chloro-6-fluoro-4-(2-(((2R,7aS)-2-fluorohexahy dro-1H-pyrrolizin-7a-yl)methoxy)-4-(2-thia-7-azaspiro[4.5]decan-7-yl)-5,6-dihydropyrido[3,4-d]pyrimidin-7(8H)-yl)naphthalen-2-yl 4-methylbenzenesulfonate: To a solution of 5-chloro-6-fluoro-4-(2-(((2R,7aS)-2-fluorotetrahydro-1H-pyrrolizin-7a(5H)-yl)methoxy)-4-(tosyloxy)-5,8-dihydropyrido[3,4-d]pyrimidin-7(6H)-yl)naphthalen-2-yl 4-methylbenzenesulfonate (75.0 mg, 1.0 equiv) and 2-thia-9-azaspiro[4.5]decane (29.1 mg, 2.0 equiv) in DMF (0.25 mL) were added N,N-diethylpropan-2-amine (59.7 mg, 5.0 equiv) and 4 Å MS (40.0 mg). The mixture was stirred at 40° C. for 12 hours. The mixture was diluted with water (10 mL) and extracted with ethyl acetate (3×10 mL). The combined organic layers were dried over anhydrous sodium sulfate, concentrated, and purified by reversed phase flash chromatography (C18, 0.1% formic acid condition) to afford the title compound (70.0 mg, 92% yield) as white solid; LCMS (ESI, M+1): m/z=796.3

Step B. 5-chloro-6-fluoro-4-(2-(((2R,7aS)-2-fluorohexahydro-1H-pyrrolizin-7a-yll)methoxy)-4-(2-thia-7-azaspiro[4.5]decan-7-yl)-5,6-dihydropyrido[3,4-d]pyrimidin-7(8H)-yl)naphthalen-2-ol: To a solution of 5-chloro-6-fluoro-4-(2-(((2R,7aS)-2-fluorohexahydro-1H-pyrrolizin-7a-yl)methoxy)-4-(2-thia-7-azaspiro[4.5]decan-7-yl)-5,6-dihydropyrido[3,4-d]pyrimidin-7(8H)-yl)naphthalen-2-yl 4-methylbenzenesulfonate (60.0 mg, 1.0 equiv) in MeOH (1.0 mL) was added LiOH•H₂O (94.8 mg, 30 equiv). The mixture was stirred at 20° C. for 12 hours. Water (10 mL) was added to the reaction at 0° C. and extracted with ethyl acetate (3×10 mL). The combined organic layers were washed with saturated brine (3×10 mL), dried over Na₂SO₄, concentrated, and purified by prep-HPLC (column: Waters Xbridge 150×25 mm×5 μm; mobile phase: phase A: water (FA), phase B: ACN]; B %: 65%-95% over 8 min) to afford the title compound (13.9 mg, 38% yield) as white solid. ¹H NMR (400 MHz, METHANOL-d₄) δ=7.66-7.58 (m, 1H), 7.32-7.23 (m, 1H), 6.98-6.90 (m, 2H), 5.42-5.18 (m, 1H), 4.30-4.17 (m, 2H), 3.97 (br d, J=13.2 Hz, 2H), 3.84-3.42 (m, 4H), 3.29-2.98 (m, 6H), 2.97-2.72 (m, 3H), 2.71-2.49 (m, 3H), 2.42-2.08 (m, 3H), 2.06-1.61 (m, 9H);LCMS (ESI, M+1): m/z=642.3

### Example 465

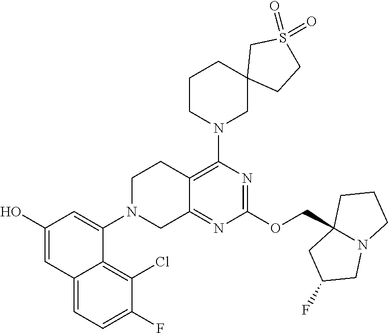 [SMILES: O=S1(=O)CCC2(CCCN(c3nc(OC[C@@]45CCCN4C[C@H](F)C5)nc4c3CCN(c3cc(O)cc5ccc(F)c(Cl)c35)C4)C2)C1]

### 7-(7-(8-chloro-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-2-thia-7-azaspiro[4.5]decane 2,2-dioxide

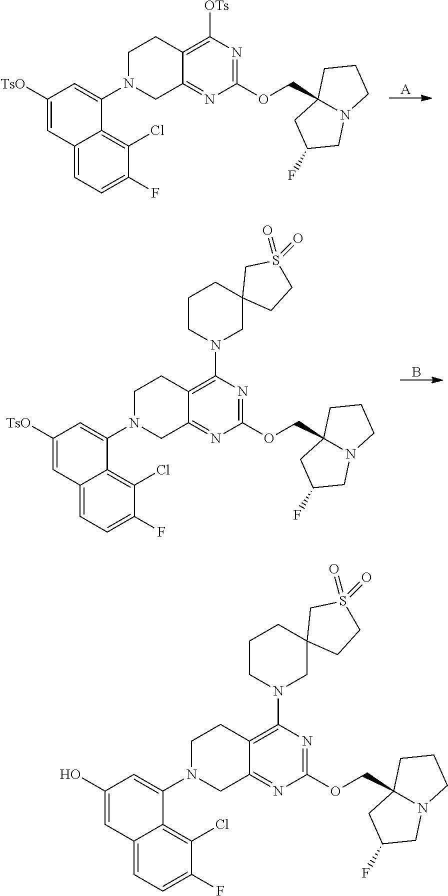

Synthesized according to Example 464. The title compound was obtained as white solid. ¹H NMR (400 MHz, METHANOL-d₄) δ=7.66-7.59 (m, 1H), 7.28 (t, J=8.8 Hz, 1H), 6.97-6.93 (m, 2H), 5.46-5.42 (m, 1H), 4.21-3.77 (m, 5H), 3.74-3.36 (m, 5H), 3.28-2.95 (m, 8H), 2.71-2.57 (m, 1H), 2.45-1.61 (m, 13H); LCMS (ESI, M+1): m/z=674.3.

### Example 466

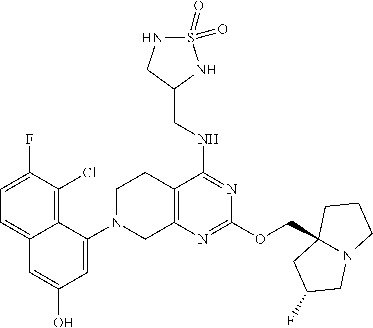 [SMILES: O=S1(=O)NCC(CNc2nc(OC[C@@]34CCCN3C[C@H](F)C4)nc3c2CCN(c2cc(O)cc4ccc(F)c(Cl)c24)C3)N1]

### 3-(((7-(8-chloro-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)amino)methyl)-1,2,5-thiadiazolidine 1,1-dioxide

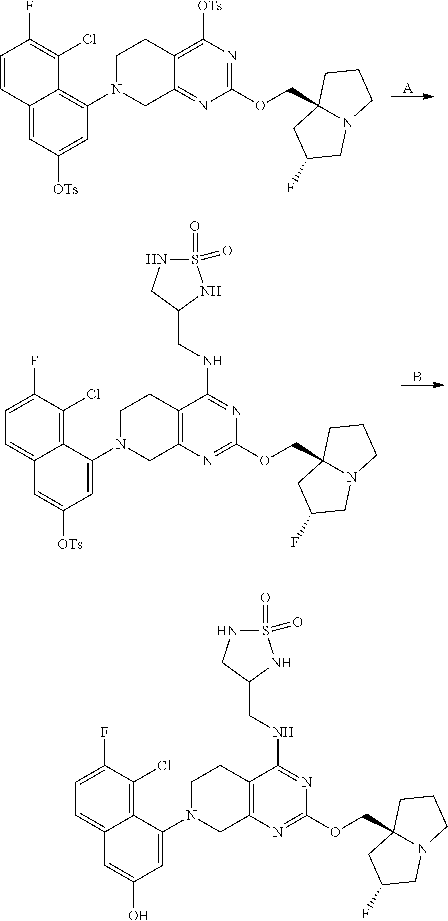

Synthesized according to Example 464. The title compound was obtained as pink solid; ¹H NMR (400 MHz, dimethylsulfoxide-d₆) δ=10.02-9.81 (m, 1H), 7.80-7.68 (m, 1H), 7.49-7.39 (m, 1H), 7.19-7.04 (m, 1H), 7.03-6.83 (m, 4H), 5.36-5.14 (m, 1H), 3.99-3.80 (m, 4H), 3.74-3.63 (m, 1H), 3.39 (br s, 4H), 3.25-3.01 (m, 6H), 3.00 (s, 1H), 2.86-2.78 (m, 1H), 2.11-2.06 (m, 1H), 2.05-1.94 (m, 2H), 1.88-1.69 (m, 3H); ¹⁹F NMR (377 MHz, dimethylsulfoxide-d₆) 6=-116.107, -171.945; LCMS (ESI, M+1). m/z=636.5.

### Example 467

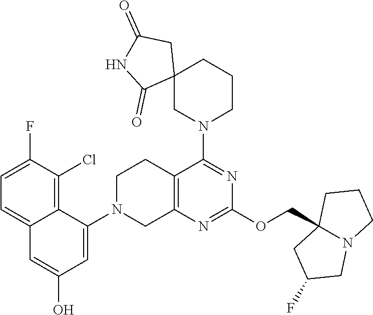 [SMILES: O=C1CC2(CCCN(c3nc(OC[C@@]45CCCN4C[C@H](F)C5)nc4c3CCN(c3cc(O)cc5ccc(F)c(Cl)c35)C4)C2)C(=O)N1]

### 7-(7-(8-chloro-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-2,7-diazaspiro[4.5]decane-1,3-dione

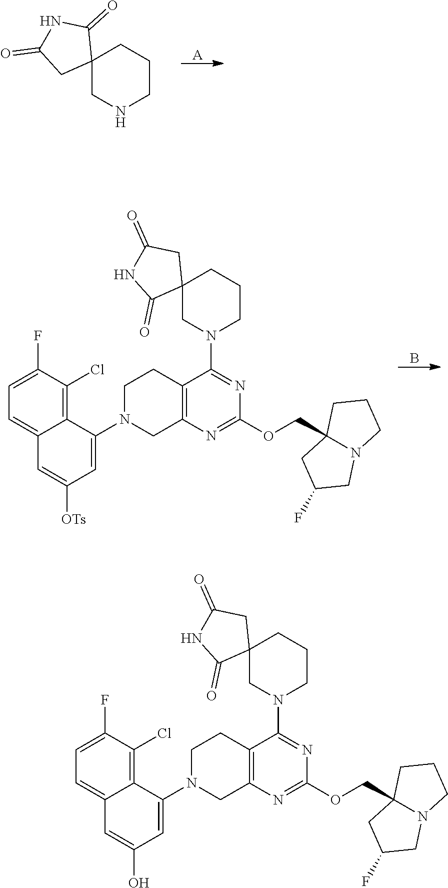

Synthesized according to Example 464. The title compound was contained as yellow solid (FA salt). ¹H NMR (400 MHz, methanol-d₄) δ=7.62 (dd, J=5.6, 9.2 Hz, 1H), 7.33-7.23 (m, 1H), 7.00-6.88 (m, 2H), 5.55-5.25 (m, 1H), 4.51-4.16 (m, 4H), 4.15-3.87 (m, 2H), 3.72-3.35 (m, 6H), 3.23-2.95 (m, 4H), 2.70-2.61 (m, 2H), 2.52-2.21 (m, 3H), 2.20-1.98 (m, 4H), 1.97-1.76 (m, 3H); LCMS (ESI, M+1): m/z=653.1.

### Example 468

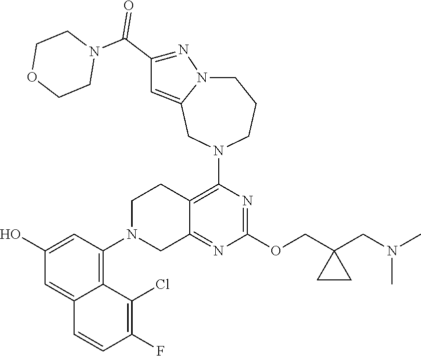 [SMILES: CN(C)CC1(COc2nc3c(c(N4CCCn5nc(C(=O)N6CCOCC6)cc5C4)n2)CCN(c2cc(O)cc4ccc(F)c(Cl)c24)C3)CC1]

### (5-(7-(8-chloro-7-fluoro-3-hydroxynaphthalen-1-yl)-2-((l-((dimethylamino)methyl)cyclopropyl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(morpholino)methanone

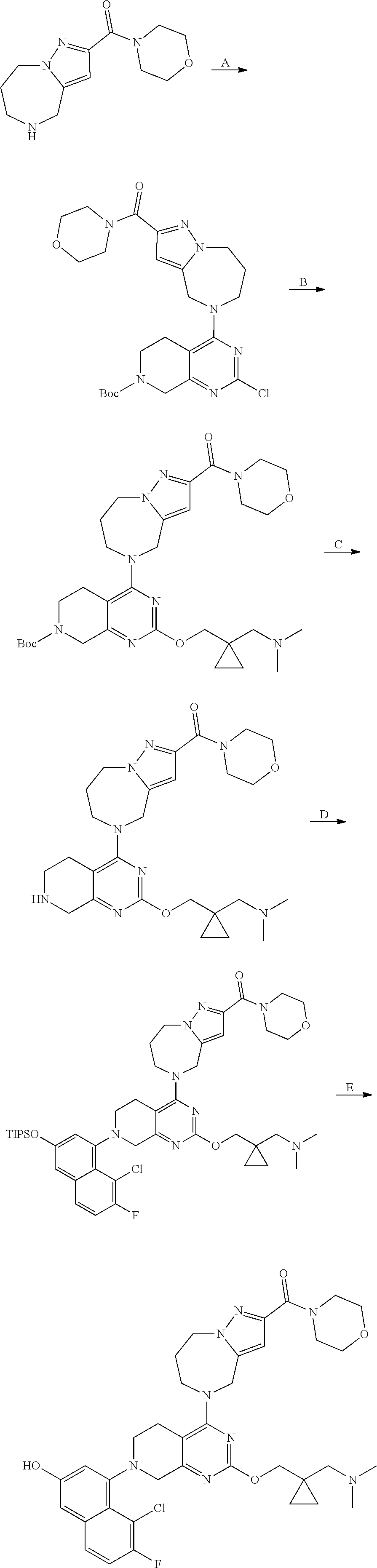

Step A. tert-butyl 2-chloro-4-(2-(morpholine-4-carbonyl)-7,8-dihydro-4H-pyrazolo[1,5-a][1,4]diazepin-5(6H)-yl)-5,6-dihydropyrido[3,4-d]pyrimidine-7(8H)-carboxylate: To a mixture of tert-butyl 2,4-dichloro-5,6-dihydropyrido[3,4-d]pyrimidine-7(8H)-carboxylate (0.05 g, 1.0 equiv) and morpholino(5,6,7,8-tetrahydro-4H-pyrazolo[1,5-a][1,4]diazepin-2-yl)methanone (49.4 mg, 1.2 equiv) in DMF (1.0 mL) were added DIEA (127 mg, 6.0 equiv) and 4 Å molecular sieve (50 mg, 1.0 equiv) under nitrogen. The reaction was stirred at 40° C. for 6 hours. The mixture was filtered and purified by reversed phase flash chromatography [C18, 0.1% formic acid condition] to afford the title compound (70.0 mg, 82% yield) as white solid. LCMS (ESI, M-55): m/z=462.0.

Step B. tert-butyl 2-((1-((dimethylamino)methyl)cyclopropyl)methoxy)-4-(2-(morpholine-4-carbonyl)-7,8-dihydro-4H-pyrazolo[1,5-a][1,4]diazepin-5(6H)-yl)-5,6-dihydropyrido[3,4-d]pyrimidine-7(8H)-carboxylate: To a mixture of tert-butyl 2-chloro-4-(2-(morpholine-4-carbonyl)-7,8-dihydro-4H-pyrazolo[1,5-a][1,4]diazepin-5(6H)-yl)-5,6-dihydropyrido[3,4-d]pyrimidine-7(8H)-carboxylate (0.50 g, 1.0 equiv) and (1-((dimethylamino)methyl)cyclopropyl)methanol (150 mg, 1.2 equiv) in toluene (5.0 mL) were added Cs₂CO₃ (943 mg, 3 equiv), BINAP (120 mg, 0.2 equiv), and Pd(OAc)₂ (21.7 mg, 0.1 equiv). The reaction was degassed and purged with nitrogen 3 times. The reaction was heated to 110° C. and stirred for 2 hours. The mixture was diluted with water (10 mL) and extracted with ethyl acetate (3×5 mL). The combined organic layers were washed with brine (10 mL), dried over anhydrous sodium sulfate, filtered, and purified by reversed phase flash chromatography [C18, 0.1% formic acid condition] to afford the title compound (315 mg, 51% yield) as white solid; ¹H NMR (400 MHz, dimethylsulfoxide-d₆) δ=6.56 (s, 1H), 4.73 (s, 2H), 4.48-4.39 (m, 2H), 4.26 (br s, 2H), 4.02-4.00 (m, 2H), 3.99-3.81 (m, 4H), 3.58 (br d, J=0.8 Hz, 6H), 3.45 (br s, 2H), 2.68-2.63 (m, 2H), 2.17 (s, 2H), 2.13 (s, 6H), 2.03 (br s, 2H), 1.44 (s, 9H), 0.58-0.52 (m, 2H), 0.38-0.31 (m, 2H); LCMS (ESI, M+1): m/z=611.5.

Step C. (5-(2-((1-((dimethylamino)methyl)cyclopropyl)methoxy)-5,6,7,8-tetrahydropyrido[3,4-d] pyrimidin-4-yl)-5,6,7,8-tetrahydro-4H-pyrazolo[1,5-a][1,4]diazepin-2-yl)(morpholino)methanone: To a solution of tert-butyl 2-((1-((dimethylamino)methyl)cyclopropyl)methoxy)-4-(2-(morpholine-4-carbonyl)-7,8-dihydro-4H-pyrazolo[1,5-a][1,4]diazepin-5(6H)-yl)-5,6-dihydropyrido[3,4-d]pyrimidine-7(8H)-carboxylate (0.31 g, 1 equiv) in DCM (3.0 mL) was added TFA (4.62 g, 79 equiv) at 0° C. under nitrogen. The mixture was stirred at 20° C. for 2 hours. The mixture was concentrated and adjusted to pH=10 with saturated Na₂CO₃ aqueous solution and 15% NaOH aqueous solution, extracted with a mixture solution (dichloromethane/methanol 10/1, 8 mL×3), dried over anhydrous sodium sulfate, filtered, and purified by reversed phase flash chromatography [C18, 0.1% formic acid condition] to afford the title compound (170 mg, 63% yield) as white solid; LCMS (ESI, M+1): m/z=511.4

Step D. (5-(7-(8-chloro-7-fluoro-3-((triisopropylsilyll)oxy)naphthalen-1-yl)-2-((1-((dimethylamino)methyl)cyclopropyl)methoxy)-5,6,7,8-tetrahydropyrido[3,4-d] pyrimidin-4-yl)-5,6,7,8-tetrahydro-4H-pyrazolo[1,5-a][1,4]diazepin-2-yl)(morpholino)methanone: To a mixture of (5-(2-((1-((dimethylamino)methyl)cyclopropyl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(morpholino)methanone (120 mg, 1.0 equiv) and 8-chloro-7-fluoro-3-((triisopropylsilyl)oxy)naphthalen-1-yl trifluoromethanesulfonate (141 mg, 1.2 equiv) in toluene (3 mL) were added Pd₂(dba)₃ (21.5 mg, 0.1 equiv), RuPhos (21.9 mg, 0.2 equiv), and Cs₂CO₃ (230 mg, 3.0 equiv). The suspension was degassed under vacuum and purged with nitrogen several times. The reaction was stirred at 100° C. for 12 hours. The reaction was filtered and purified by reversed phase chromatography [C18, 0.1% formic acid condition] to afford the title compound (55 mg, 63% yield) as yellow oil; LCMS (ESI, M+1): m/z=861.6.

Step E. (5-(7-(8-chloro-7-fluoro-3-hydroxynaphthalen-1-yl)-2-((1-((dimethylamino)methyl)cyclopropyl)methoxy)-5,6,7,8-tetrahydropyrido[3,4-d] pyrimidin-4-yl)-5,6,7,8-tetrahydro-4H-pyrazolo[1,5-a][1,4]diazepin-2-yl)(morpholino)methanone: To a solution of (5-(7-(8-chloro-7-fluoro-3-((triisopropylsilyl)oxy)naphthalen-1-yl)-2-((1-((dimethylamino)methyl)cyclopropyl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(morpholino)methanone (68 mg, 1.0 equiv) in DMF (0.5 mL) was added CsF (59.9 mg, 5.0 equiv). The reaction was stirred at 20° C. for 1 hour. The reaction was filtered, purified by prep-HPLC [Waters xbridge 150×25 mm×10 μm; A: water (NH₄HCO₃), B: ACN; B %: 34%-64% over 9 min] and lyophilized to afford the title compound (8.37 mg, 14% yield) as orange solid; ¹H NMR (400 MHz, methanol-d₄) δ=7.66-7.58 (m, 1H), 7.27 (t, J=8.8 Hz, 1H), 6.99-6.89 (m, 2H), 6.61-6.52 (m, 1H), 5.03-4.93 (m, 1H), 4.80-4.75 (m, 1H), 4.58-4.44 (m, 2H), 4.26-4.08 (m, 4H), 4.05-3.89 (m, 3H), 3.80-3.50 (m, 8H), 3.27-3.21 (m, 1H), 3.18-3.05 (m, 1H), 2.71-2.59 (m, 1H), 2.48-2.38 (m, 2H), 2.33-2.16 (m, 7H), 2.13-1.99 (m, 1H), 0.72-0.57 (m, 2H), 0.54-0.40 (m, 2H); ¹⁹F NMR (400 MHz, methanol-d₄) 6=-117.438; LCMS (ESI, M+1): m/z=705.4.

### Example 469

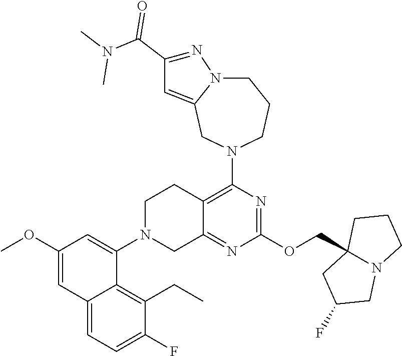 [SMILES: CCc1c(F)ccc2cc(OC)cc(N3CCc4c(nc(OC[C@@]56CCCN5C[C@H](F)C6)nc4N4CCCn5nc(C(=O)N(C)C)cc5C4)C3)c12]

### 5-(7-(8-ethyl-7-fluoro-3-methoxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide

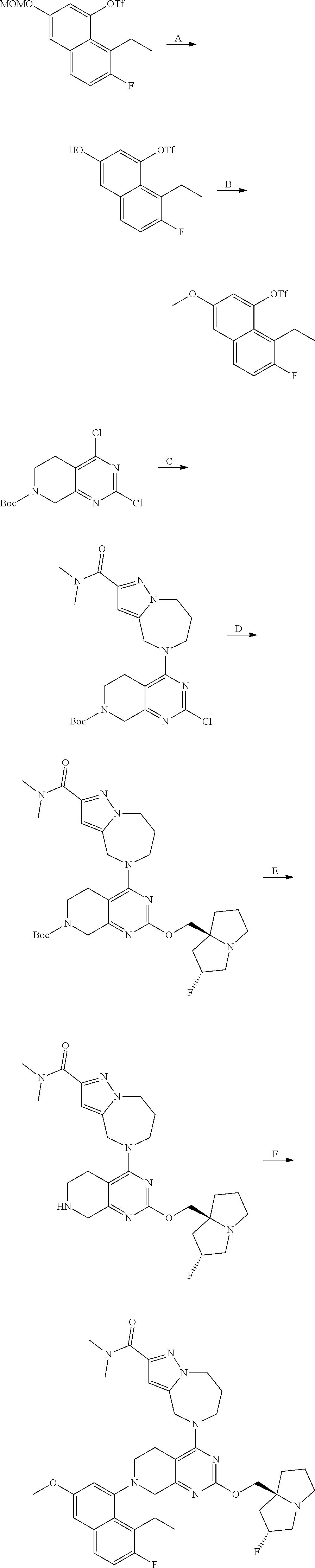

Step A. 8-ethyl-7-fluoro-3-hydroxynaphthalen-1-yl trifluoromethanesulfonate: To a solution of 8-ethyl-7-fluoro-3-(methoxymethoxy)naphthalen-1-yl trifluoromethanesulfonate (10.0 g, 1.0 equiv) in ACN (50 mL) was added HCl•dioxane (4 M, 50 mL) at 0° C. The mixture was stirred at 25° C. for 1 hour. The reaction was concentrated. The pH of the residue was adjusted to 10 by saturated NaOH solution (30 mL) at 0° C. The reaction was extracted with DCM (3×100 mL). The combined organic layers were dried over anhydrous sodium sulfate and concentrated to afford the title compound (8.8 g, crude) as yellow solid.

Step B. 8-ethyl-7-fluoro-3-methoxynaphthalen-1-yl trifluoromethanesulfonate: To a solution of 8-ethyl-7-fluoro-3-hydroxynaphthalen-1-yl trifluoromethanesulfonate (8.00 g, 1.0 equiv) in MeOH (40 mL) was added diazomethyl(trimethyl)silane (13.5 g, 5.0 equiv). The mixture was stirred at 15° C. for 12 hours. The reaction was concentrated in vacuum to give a residue. Then the residue was purified by silica gel chromatography to afford the title compound (8.00 g, 91% yield) as white solid; ¹H NMR (400 MHz, METHANOL-d₄) δ=7.77-7.57 (m, 1H), 7.23 (s, 3H), 3.95-3.78 (m, 3H), 3.25-3.10 (m, 2H), 1.20-1.09 (m, 3H).

Step C. tert-butyl 2-chloro-4-(2-(dimethylcarbamoyl)-7,8-dihydro-4H-pyrazolo[1,5-a][1,4]diazepin-5(6H)-yl)-5,6-dihydropyrido[3,4-d]pyrimidine-7(8H)-carboxylate: To a mixture of tert-butyl 2,4-dichloro-5,6-dihydropyrido[3,4-d]pyrimidine-7(8H)-carboxylate (16.0 g, 1.0 equiv) and N,N-dimethyl-5,6,7,8-tetrahydro-4H-pyrazolo[1,5-a][1,4]diazepine-2-carboxamide (16.4 g, 1.5 equiv) in DMF (40 mL) was added DIEA(20.4 g, 3.0 equiv), and then the mixture was stirred at 25° C. for 12 hours. The reaction was filtered. The filtrate was purified with reversed phase flash chromatography [C18, 0.1% formic acid condition] to afford the title compound (20.0 g, 77% yield) as yellow solid; LCMS (ESI, M+1): m/z=476.2.

Step D. tert-butyl 4-(2-(dimethylcarbamoyl)-7,8-dihydro-4H-pyrazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidine-7(8H)-carboxylate: A mixture of tert-butyl 2-chloro-4-(2-(dimethylcarbamoyl)-7,8-dihydro-4H-pyrazolo[1,5-a][1,4]diazepin-5(6H)-yl)-5,6-dihydropyrido[3,4-d]pyrimidine-7(8H)-carboxylate (20.0 g, 1.0 equiv), ((2R,7aS)-2-fluorohexahydro-1H-pyrrolizin-7a-yl)methanol (10.0 g, 1.5 equiv), Pd(OAc)₂ (943 mg, 0.1 equiv), CS₂CO₃ (27.4 g, 2.0 equiv), and BINAP (27.4 g, 2.0 equiv) in toluene (100 mL) was degassed and purged with N₂ 3 times, and then the mixture was stirred at 110° C. for 12 hours under N₂ atmosphere. The reaction was filtered, concentrated, and purified by reversed phase flash chromatography [C18, 0.1% formic acid condition] to afford the title compound (18.0 g, 71% yield) as white solid; LCMS (ESI, M+1): m/z=599.2.

Step E. 5-(2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide: To a solution of tert-butyl 4-(2-(dimethylcarbamoyl)-7,8-dihydro-4H-pyrazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidine-7(8H)-carboxylate (18.0 g, 1.0 equiv) in MeOH (40 mL) was added HCl•dioxane (4 M, 73 mL) at 0° C. The mixture was stirred at 25° C. for 1 hour. The reaction was concentrated. The pH of the residue was adjusted to 10 by saturated NaOH solution (20 mL) at 0° C. The reaction was extracted with DCM (3×100 mL). The combined organic layers were dried over anhydrous sodium sulfate and concentrated to afford the title compound (14.0 g, crude) as yellow solid; LCMS (ESI, M+1): m/z=499.5.

Step F. 5-(7-(8-ethyl-7-fluoro-3-methoxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide: A mixture of 5-(2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide (7.00 g, 1.0 equiv), 8-ethyl-7-fluoro-3-methoxynaphthalen-1-yl trifluoromethanesulfonate (7.42 g, 1.5 equiv), Pd₂(dba)₃ (1.93 g, 0.15 equiv), Cs₂CO₃ (13.7 g, 3.0 equiv), and Xantphos (2.03 g, 0.25 equiv) in toluene (70 mL) was degassed and purged with N₂ 3 times, and then the mixture was stirred at 110° C. for 12 hours under N₂ atmosphere. The mixture was quenched with H₂O (50 mL) and extracted with EtOAc (3×200 mL). The combined organic layers were dried over anhydrous sodium sulfate, concentrated, and purified by reversed phase flash chromatography [C18, 0.1% formic acid condition] to afford the title compound (8.20 g, 90% yield) as white solid; 1H NMR (400 MHz, METHANOL-d₄) δ=7.69-7.56 (m, 1H), 7.21-7.13 (m, 1H), 7.11-6.92 (m, 2H), 6.69-6.54 (m, 1H), 5.32-5.14 (m, 1H), 5.03-4.94 (m, 1H), 4.84-4.74 (m, 1H), 4.59-4.44 (m, 2H), 4.23-3.91 (m, 6H), 3.89-3.79 (m, 3H), 3.66-3.56 (m, 1H), 3.50-3.32 (m, 4H), 3.26-3.16 (m, 3H), 3.14-3.03 (m, 5H), 3.00-2.91 (m, 1H), 2.71-2.60 (m, 1H), 2.01 (s, 6H), 1.96-1.78 (m, 3H), 1.16-1.04 (m, 3H); LCMS (ESI, M+1): m/z=701.3.

### Example 470

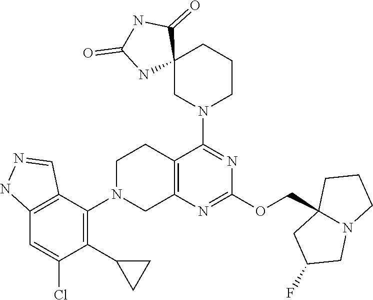 [SMILES: O=C1NC(=O)[C@]2(CCCN(c3nc(OC[C@@]45CCCN4C[C@H](F)C5)nc4c3CCN(c3c(C5CC5)c(Cl)cc5[nH]ncc35)C4)C2)N1]

### (R)-7-(7-(6-chloro-5-cyclopropyl-1H-indazol-4-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione

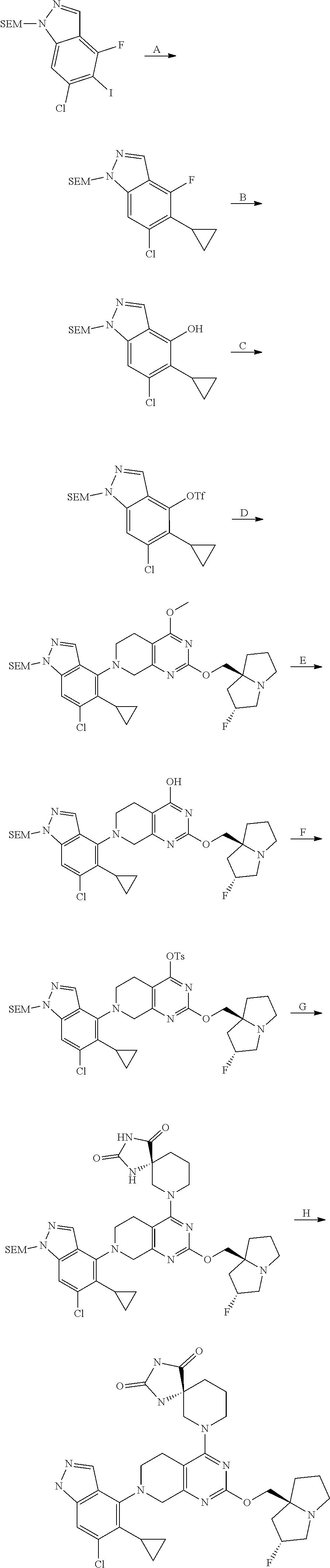

Step A. 6-chloro-5-cyclopropyl-4-fluoro-1-((2-(trimethylsilyl)ethoxy)methyl)-1H-indazole: To a solution of 2-[(6-chloro-4-fluoro-5-iodo-indazol-1-yl)methoxy]ethyl-trimethyl-silane (8.0 g, 1.0 equiv) and cyclopropylboronic acid (3.22 g, 2.0 equiv) in dioxane (80 mL) were added Pd(dppf)Cl₂ (1.37 g, 0.1 equiv) and a solution of K₃PO₄ (1.5 M, 37.5 mL, 3.0 equiv) in water. The reaction was degassed and purged with N₂ three times and stirred at 100° C. for 12 hours under N₂. The mixture was diluted with water (80 mL) and extracted with ethyl acetate (2×60 mL). The combined organic layers were dried over anhydrous sodium sulfate, concentrated, and purified by column chromatography [SiO₂, petroleum ether/ethyl acetate=I/O to 10/1] to afford the title compound (4.10 g, 58% yield) as yellow oil. ¹H NMR (400 MHz, CDCl₃-d) δ=8.00 (d, J=0.8 Hz, 1H), 7.44 (s, 1H), 5.79-5.55 (m, 2H), 3.62-3.48 (m, 2H), 1.90-1.75 (m, 1H), 1.16-1.02 (m, 2H), 0.96-0.80 (m, 4H), 0.01 (s, 9H); LCMS (ESI, M+1): m/z=341.1.

Step B. 6-chloro-5-cyclopropyl-1-((2-(trimethylsilyl)ethoxy)methyl)-1H-indazol-4-ol: To a solution of 2-[(6-chloro-5-cyclopropyl-4-fluoro-indazol-2-yl)methoxy]ethyl-trimethyl-silane (4.10 g, 1.0 equiv) and 2-methylsulfonylethanol (2.20 g, 1.5 equiv) in DMF (50 mL) was added NaH (2.40 g, 60% purity, 5.0 equiv) in portions at 0° C. Then the reaction was stirred at 25° C. for 0.5 hours and 40° C. for 11.5 hours. The mixture was quenched with H₂O (50 mL) and extracted with ethyl acetate (2×10 mL). The combined organic layers were dried over anhydrous sodium sulfate, concentrated and purified with reversed phase flash chromatography [0.1% formic acid condition] to afford the title compound (2.0 g, 50% yield) as yellow oil. ¹H NMR (400 MHz, CDCl₃-d) δ=8.12-7.98 (m, 1H), 7.20 (s, 1H), 6.54 (s, 1H), 5.80-5.58 (m, 2H), 3.66-3.41 (m, 2H), 1.78-1.60 (m, 1H), 1.27-1.14 (m, 2H), 0.98-0.83 (m, 2H), 0.80-0.69 (m, 2H), −0.04 (s, 9H); LCMS (ESI, M+1): m/z=339.3.

Step C. 6-chloro-5-cyclopropyl-1-((2-(trimethylsilyl)ethoxy)methyl)-1H-indazol-4-yl trifluoromethanesulfonate: To a solution of 6-chloro-5-cyclopropyl-1-(2-trimethylsilylethoxymethyl)indazol-4-ol (0.80 g, 1.0 equiv), 4 Å molecular sieve (100 mg) and DIEA (915 mg, 3.0 equiv) in DCM (8 mL) was added Tf₂O (999 mg, 1.5 equiv). The mixture was degassed and purged with N₂ three times and stirred at −40° C. for 0.5 hours. The mixture was diluted with water (3 mL) and extracted with ethyl acetate (2×5 mL). The combined organic layers were dried over anhydrous sodium sulfate, concentrated, and purified with column chromatography [SiO2, petroleum ether/ethyl acetate=I/O to 10/1] to afford the title compound (740 mg, 62% yield) as yellow oil. ¹H NMR (400 MHz, CDCl₃-d) δ=7.98 (s, 1H), 7.70 (s, 1H), 5.70 (s, 2H), 3.55 (t, J=8.4 Hz, 2H), 1.94-1.82 (m, 1H), 1.31-1.19 (m, 2H), 0.90 (t, J=8.4 Hz, 2H), 0.81-0.70 (m, 2H), −0.05 (s, 9H); LCMS (ESI, M+1): m/z=471.1.

Step D. 7-(6-chloro-5-cyclopropyl-1-((2-(trimethylsilyl)ethoxy)methyl)-1H-indazol-4-yl)-2-(((2R,7aS)-2-fluorohexahydro-1H-pyrrolizin-7a-yl)methoxy)-4-methoxy-5,6,7,8-tetrahydropyrido[3,4-d]pyrimidine: A mixture of 2-(((2R,7aS)-2-fluorotetrahydro-1H-pyrrolizin-7a(5H)-yl)methoxy)-4-methoxy-5,6,7,8-tetrahydropyrido[3,4-d]pyrimidine (700 mg, 1.0 equiv), [6-chloro-5-cyclopropyl-1-(2-trimethylsilylethoxymethyl)indazol-4-yl]trifluoromethanesulfonate (1.02 g, 1.0 equiv), Cs₂CO₃ (2.12 g, 3.0 equiv), and Xantphos Pd G4 (209 mg, 0.1 equiv) in dioxane (5 mL) was degassed and purged with N₂ 3 times, and then the reaction was stirred at 90° C. for 36 hours under N₂ atmosphere. The mixture was filtered and the filtrate was diluted with water (50 mL) and extracted with ethyl acetate (2×20 mL). The combined organic layers were washed with brine (20 mL), dried over anhydrous sodium sulfate, concentrated, and purified by prep-HPLC (column: Welch Ultimate XB-SiOH 250×50×10 um; mobile phase: [Hexane-EtOH (0.1% NH₃.H₂O)];B %: 1%-15%,15 min) to afford the title compound (350 mg, 20% yield) as yellow oil; H NMR (400 MHz, CHLOROFORM-d) δ=8.01-7.93 (m, 1H), 7.34 (s, 1H), 5.63 (s, 2H), 5.57-5.40 (m, 1H), 4.50 (s, 2H), 4.06-3.98 (m, 5H), 3.81-3.77 (m, 1H), 3.59-3.52 (m, 2H), 3.33-3.19 (m, 2H), 2.79-2.72 (m, 2H), 2.32-2.14 (m, 4H), 1.83-1.71 (m, 1H), 1.52-1.41 (m, 2H), 1.28-1.25 (m, 2H), 1.08-0.99 (m, 2H), 0.95-0.86 (m, 3H), 0.70-0.59 (m, 2H), −0.04 (s, 9H); LCMS (ESI, M+1): m/z=643.4.

Step E. 7-(6-chloro-5-cyclopropyl-1-((2-(trimethylsilyl)ethoxy)methyl)-1H-indazol-4-yl)-2-(((2R,7aS)-2-fluorohexahydro-1H-pyrrolizin-7a-yl)methoxy)-5,6,7,8-tetrahydropyrido[3,4-d]pyrimidin-4-ol: To a solution of ethanethiol (0.44 g, 17 equiv) in DMAc (3 mL) was added NaH (32.0 mg, 2.0 equiv) at 0° C. under N₂ atmosphere. After addition, the mixture was stirred at 0° C. for 0.5 hours, and then 7-(6-chloro-5-cyclopropyl-1-((2-(trimethylsilyl)ethoxy)methyl)-1H-indazol-4-yl)-2-(((2R,7aS)-2-fluorotetrahydro-1H-pyrrolizin-7a(5H)-yl)methoxy)-4-methoxy-5,6,7,8-tetrahydropyrido[3,4-d]pyrimidine (260 mg, 1.0 equiv) in DMAc (1 mL) was added dropwise at 25° C. The reaction was stirred at 60° C. for 1 hour. The mixture was quenched with water (20 mL) at 0° C. for 2 minutes and extracted with ethyl acetate (3×10 mL). The combined organic layers were dried over anhydrous sodium sulfate, concentrated and purified by reversed phase flash chromatography [water (0.1% FA)/acetonitrile] to afford the title compound (110 mg, 41% yield) as yellow solid; LCMS (ESI, M+1): m/z=629.2.

Step F. 7-(6-chloro-5-cyclopropyl-1-((2-(trimethylsilyl)ethoxy)methyl)-1H-indazol-4-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate: To a solution of 7-(6-chloro-5-cyclopropyl-1-((2-(trimethylsilyl)ethoxy)methyl)-1H-indazol-4-yl)-2-(((2R,7aS)-2-fluorotetrahydro-1H-pyrrolizin-7a(5H)-yl)methoxy)-5,6,7,8-tetrahydropyrido[3,4-d]pyrimidin-4-ol (155 mg, 1.0 equiv) in DCM (1 mL) were added N,N-diethylpropan-2-amine (96.0 mg, 3.0 equiv) and DMAP (3.0 mg, 0.1 equiv) at 0° C. After addition, the mixture was stirred at this temperature for 10 minutes, and then 4-methylbenzenesulfonyl chloride (70 mg, 1.5 equiv) in DCM (1 mL) was added dropwise at 0° C. The resulting mixture was stirred at 25° C. for 2 hours. The reaction was diluted with water (20 mL) and extracted with EtOAc (10 mL). The combined organic layers were washed with brine (10 mL), dried over anhydrous sodium sulfate, concentrated, and purified by reversed phase flash chromatography [water (0.1% FA)/acetonitrile] to afford the title compound (120 mg, 52% yield) as yellow oil; LCMS (ESI, M+1): m/z=783.2.

Step G. (R)-7-(7-(6-chloro-5-cyclopropyl-1-((2-(trimethylsilyl)ethoxy)methyl)-1H-indazol-4-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione: To a solution of (5R)-1,3,9-triazaspiro[4.5]decane-2,4-dione (77.4 mg, 3 equiv) in DMF (1.0 mL) were added N,N-diethylpropan-2-amine (39.6 mg, 2.0 equiv), 4 Å molecular sieve (20 mg), and 7-(6-chloro-5-cyclopropyl-1-((2-(trimethylsilyl)ethoxy)methyl)-1H-indazol-4-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (120 mg, 1.0 equiv). The reaction was stirred at 40° C. for 48 hours. The mixture was filtered and the filtrate was purified by reversed phase flash chromatography [water (0.1% FA)/acetonitrile] to afford the title compound (115 mg, 89% yield) as white solid; LCMS (ESI, M+1): m/z=780.5.

Step H. (R)-7-(7-(6-chloro-5-cyclopropyl-1H-indazol-4-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione: To a solution of (R)-7-(7-(6-chloro-5-cyclopropyl-1-((2-(trimethylsilyl)ethoxy)methyl)-1H-indazol-4-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (100 mg, 1.0 equiv) in DCM (2 mL) was added TFA (7.70 g) at 0° C. The reaction was stirred at 0° C. for 1 hour. Then the mixture was stirred at 20° C. for 2 hours. The mixture was concentrated, purified by prep-HPLC [column: Phenomenex luna C18 150 x 25mmx 10 um; A: water (FA)-, B: ACN, B %: 16%-46% over 10 min] followed by prep-HPLC [column: Unisil 3-100 C18 Ultra 150×50 mm×3 um; A: water (FA), B: ACN, B %: 15%-45% over 7 min] and lyophilized to afford the title compound (16 mg) as white solid (FA salt). ¹H NMR (400 MHz, METHANOL-d₄) δ=8.06 (s, 1H), 7.30 (d, J=0.8 Hz, 1H), 5.48-5.31 (m, 1H), 4.52-4.42 (m, 2H), 4.36-4.21 (m, 2H), 4.17 (br d, J=13.2 Hz, 1H), 4.02 (br d, J=12.8 Hz, 1H), 3.76 (t, J=5.2 Hz, 2H), 3.62-3.44 (m, 3H), 3.40 (d, J=13.2 Hz, 1H), 3.25-3.10 (m, 2H), 2.95-2.84 (m, 2H), 2.52-2.29 (m, 2H), 2.29-2.18 (m, 1H), 2.15-2.04 (m, 3H), 2.04-1.97 (m, 1H), 1.95-1.82 (m, 3H), 1.81-1.71 (m, 1H), 1.10-1.00 (m, 2H), 0.70 (br d, J=4.0 Hz, 2H); LCMS (ESI, M+1): m/z=650.3.

### Example 471

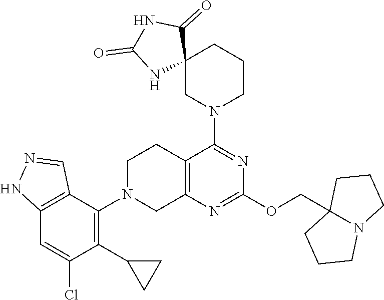 [SMILES: O=C1NC(=O)[C@]2(CCCN(c3nc(OCC45CCCN4CCC5)nc4c3CCN(c3c(C5CC5)c(Cl)cc5[nH]ncc35)C4)C2)N1]

### (R)-7-(7-(6-chloro-5-cyclopropyl-1H-indazol-4-yl)-2-((hexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione

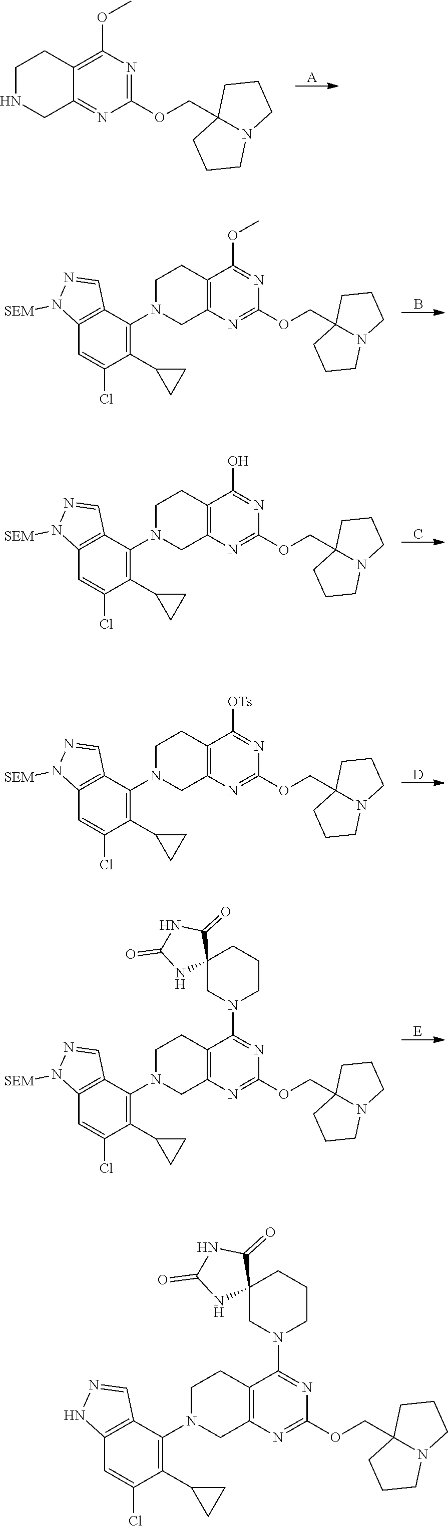

Synthesized according to Example 470 except that 2-((hexahydro-1H-pyrrolizin-7a-yl)methoxy)-4-methoxy-5,6,7,8-tetrahydropyrido[3,4-d]pyrimidine instead of 2-(((2R,7aS)-2-fluorotetrahydro-1H-pyrrolizin-7a(5H)-yl)methoxy)-4-methoxy-5,6,7,8-tetrahydropyrido[3,4-d]pyrimidine was used in step A. The title compound was obtained as yellow solid (FA salt). ¹H NMR (400 MHz, MeOH-d₄) δ 8.02 (s, 1H), 7.30 (s, 1H), 4.48 (s, 2H), 4.34 (br d, J=3.6 Hz, 2H), 4.16 (br d, J=12.8 Hz, 1H), 4.02 (br d, J=13.2 Hz, 1H), 3.77 (br t, J=5.2 Hz, 2H), 3.47-3.39 (m, 3H), 3.27-3.16 (m, 1H), 3.10-2.98 (m, 2H), 2.91 (br s, 2H), 2.24-2.16 (m, 2H), 2.14-2.02 (m, 5H), 1.98-1.84 (m, 5H), 1.81-1.73 (m, 1H), 1.09-1.00 (m, 2H), 0.71 (br d, J=3.6 Hz, 2H); LCMS (ESI, M+1): m/z=632.4.

### Example 472

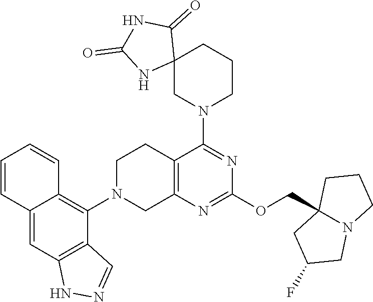 [SMILES: O=C1NC(=O)C2(CCCN(c3nc(OC[C@@]45CCCN4C[C@H](F)C5)nc4c3CCN(c3c5ccccc5cc5[nH]ncc35)C4)C2)N1]

### 7-(7-(1H-benzo[f]indazol-4-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-triazaspiro [4.5]decane-2,4-dione

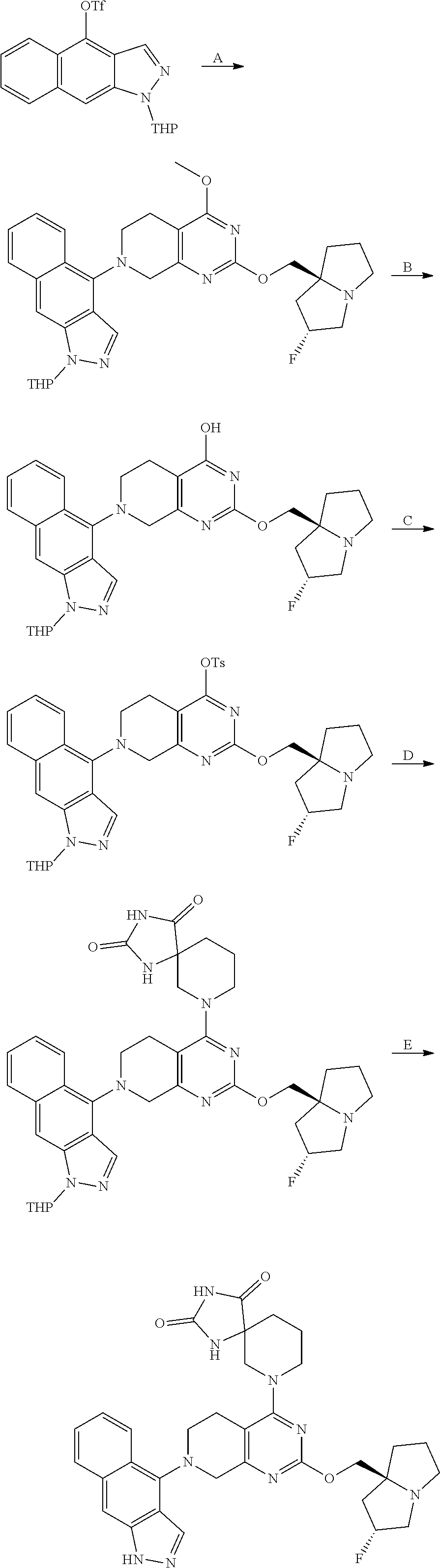

Step A. 4-(2-(((2R,7aS)-2-fluorohexahydro-1H-pyrrolizin-7a-yl)methoxy)-4-methoxy-5,6-dihydropyrido[3,4-d] pyrimidin-7(8H)-yl)-1-(tetrahydro-2H-pyran-2-yl)-1H-benzo[f]indazole: To a mixture of 2-(((2R,7aS)-2-fluorotetrahydro-1H-pyrrolizin-7a(5H)-yl)methoxy)-4-methoxy-5,6,7,8-tetrahydropyrido[3,4-d]pyrimidine (248 mg, 1.0 equiv), (1-tetrahydropyran-2-ylbenzo[f]indazol-4-yl) trifluoromethanesulfonate (400 mg, 1.3 equiv), and dicyclohexyl-[2-(2,6-diisopropoxyphenyl)phenyl]phosphane (71.7 mg, 0.2 equiv) in toluene (4 mL) were added Cs₂CO₃ (751 mg, 3.0 equiv) and Pd₂(dba)₃ (70.4 mg, 0.1 equiv). The reaction was stirred at 100° C. for 5 hours under nitrogen atmosphere. The mixture was diluted with H₂O (4 mL) and extracted with ethyl acetate (2×5 mL). The combined organic layers were washed with brine (5 mL), dried over anhydrous sodium sulfate, concentrated, and purified with reversed phase flash chromatography [water (FA 0.1%)/acetonitrile] to afford the title compound (376 mg, 82% yield) as yellow solid; LCMS (ESI, M+1): m/z=573.2.

Step B. 2-(((2R,7aS)-2-fluorohexahydro-1H-pyrrolizin-7a-yl)methoxy)-7-(1-(tetrahydro-2H-pyran-2-yl)-1H-benzo[f]indazol-4-yl)-5,6,7,8-tetrahydropyrido[3,4-d]pyrimidin-4-ol: To a solution of EtSH (940 mg, 18 equiv) in DMAc (24 mL) was added NaH (98.5 mg, 60% purity, 3.0 equiv). The reaction was stirred at 0° C. for 0.5 hours. A solution of 4-(2-(((2R,7aS)-2-fluorohexahydro-1H-pyrrolizin-7a-yl)methoxy)-4-methoxy-5,6-dihydropyrido[3,4-d]pyrimidin-7(8H)-yl)-1-(tetrahydro-2H-pyran-2-yl)-1H-benzo[f]indazole (470 mg, 1.0 equiv) in DMAC (6 mL) was added. The reaction was stirred at 60° C. for 0.5 hours. The mixture was diluted with water (20 ml). The pH was adjusted to 5 with 2 N HCl. The mixture was extracted with ethyl acetate (3×30 mL). The combined organic layers were washed with brine (20 mL), dried over anhydrous sodium sulfate, and concentrated afford the title compound (452 mg, crude) as yellow solid. LCMS (ESI, M+1): m/z=559.3.

Step C. 2-(((2R,7aS)-2-fluorohexahydro-1H-pyrrolizin-7a-yl)methoxy)-7-(1-(tetrahydro-2H-pyran-2-yl)-1H-benzo[f]indazol-4-yl)-5,6,7,8-tetrahydropyrido[3,4-d] pyrimidin-4-yl 4-methylbenzenesulfonate: To a mixture of 2-(((2R,7aS)-2-fluorohexahydro-1H-pyrrolizin-7a-yl)methoxy)-7-(1-(tetrahydro-2H-pyran-2-yl)-1H-benzo[f]indazol-4-yl)-5,6,7,8-tetrahydropyrido[3,4-d]pyrimidin-4-ol (450 mg, 1.0 equiv), DIEA (312 mg, 3.0 equiv), and DMAP (9.84 mg, 0.1 equiv) in DCM (5 mL) was added 4-methylbenzenesulfonyl chloride (177 mg, 1.1 equiv). The reaction was stirred at 25° C. for 0.5 hours. The mixture was diluted with H₂O (5 mL) and extracted with DCM (2×20 mL). The combined organic layers were washed with brine (10 mL), dried over anhydrous sodium sulfate, concentrated, and purified with reversed phase flash chromatography [water (FA 0.1%)/acetonitrile] to afford the title compound (358 mg, 58% yield) as yellow solid; LCMS (ESI, M+1): m/z=713.4.

Step D. 7-(2-(((2R,7aS)-2-fluorohexahydro-1H-pyrrolizin-7a-yl)methoxy)-7-(1-(tetrahydro-2H-pyran-2-yll)-1H-benzo[f]indazol-4-yl)-5,6,7,8-tetrahydropyrido[3,4-d]pyrimidin-4-yl)-1,3,7-triazaspiro[4.5]decane-2,4-dione: To a mixture of 2-(((2R,7aS)-2-fluorohexahydro-1H-pyrrolizin-7a-yl)methoxy)-7-(1-(tetrahydro-2H-pyran-2-yl)-1H-benzo[f]indazol-4-yl)-5,6,7,8-tetrahydropyrido[3,4-d]pyrimidin-4-yl 4-methylbenzenesulfonate (80.0 mg, 1.0 equiv), 4 Å molecular sieve (8 mg) and N,N-diethylpropan-2-amine (145 mg, 10 equiv) in DMF (0.8 mL) was added 1,3,9-triazaspiro[4.5]decane-2,4-dione (47.5 mg, 2.5 equiv). The reaction was stirred at 40° C. for 12 hours. The mixture was filtered, concentrated and purified by reversed phase flash chromatography [water (FA 0.10%)/acetonitrile] to afford the title compound (46.2 mg, 50% yield) as yellow solid; LCMS (ESI, M+1): m/z=710.4.

Step E. 7-(7-(1H-benzo[f]indazol-4-yl)-2-(((2R,7aS)-2-fluorohexahydro-1H-pyrrolizin-7a-yl)methoxy)-5,6,7,8-tetrahydropyrido[3,4-d] pyrimidin-4-yl)-1,3,7-triazaspiro[4.5]decane-2,4-dione: A mixture of 7-(2-(((2R,7aS)-2-fluorohexahydro-1H-pyrrolizin-7a-yl)methoxy)-7-(1-(tetrahydro-2H-pyran-2-yl)-1H-benzo[f]indazol-4-yl)-5,6,7,8-tetrahydropyrido[3,4-d]pyrimidin-4-yl)-1,3,7-triazaspiro[4.5]decane-2,4-dione (25.0 mg, 1.0 equiv) in TFA (1.93 g, 480 equiv) was stirred at 20° C. for 1 hour. The mixture was concentrated and purified by prep-HPLC (column: Welch Xtimate C18 150*25 mm*5 um; mobile phase: [water (TFA)-ACN]; B %: 14%-44%,10 min). The residue was purified by prep-HPLC (column: Welch Xtimate C18 150*25 mm*5 um;mobile phase: [water (ammonia hydroxide v/v)-ACN];B %: 21%) to afford the title compound (3.17 mg) as off-white solid; ¹H NMR (400 MHz, METHANOL-d₄) δ=8.40 (s, 2H), 7.91 (d, J=8.4 Hz, 1H), 7.77 (s, 1H), 7.41 (s, 1H), 7.32 (br d, J=8.8 Hz, 1H), 5.34 (br s, 1H), 4.46 (s, 2H), 4.24-4.02 (m, 4H), 3.82-3.74 (m, 2H), 3.51-3.35 (m, 2H), 3.25-3.10 (m, 4H), 3.07-2.95 (m, 2H), 2.30 (br d, J=4.4 Hz, 4H), 2.00 (br s, 6H), LCMS (ESI, M+1): m/z=626.4.

### Example 473

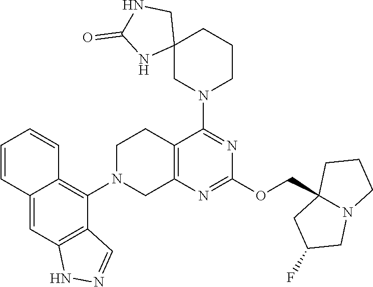 [SMILES: O=C1NCC2(CCCN(c3nc(OC[C@@]45CCCN4C[C@H](F)C5)nc4c3CCN(c3c5ccccc5cc5[nH]ncc35)C4)C2)N1]

7-(7-(1H-benzo[f]indazol-4-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-triazaspiro[4.5]decan-2-one

Synthesized according to Example 472. The title compound was obtained as yellow solid; ¹H NMR (400 MHz, METHANOL-d₄) δ=8.41-8.33 (m, 2H), 7.91 (d, J=8.0 Hz, 1H), 7.77 (s, 1H), 7.44-7.39 (m, 1H), 7.34-7.29 (m, 1H), 5.42-5.23 (m, 1H), 4.47 (s, 2H), 4.29-4.12 (m, 2H), 3.84-3.74 (m, 2H), 3.62 (br d, J=16.8 Hz, 4H), 3.50-3.36 (m, 4H), 3.17-3.02 (m, 2H), 2.32-2.12 (m, 3H), 2.10-1.95 (m, 3H), 1.93-1.63 (m, 6H); LCMS (ESI, M+1): m/z=612.3.

### Example 474

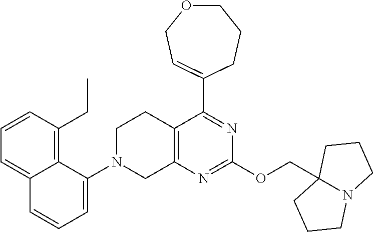 [SMILES: CCc1cccc2cccc(N3CCc4c(nc(OCC56CCCN5CCC6)nc4C4=CCOCCC4)C3)c12]

### 7-(8-ethylnaphthalen-1-yl)-2-((hexahydro-1H-pyrrolizin-7a-yl)methoxy)-4-(2,5,6,7-tetrahydrooxepin-4-yl)-5,6,7,8-tetrahydropyrido[3,4-d]pyrimidine

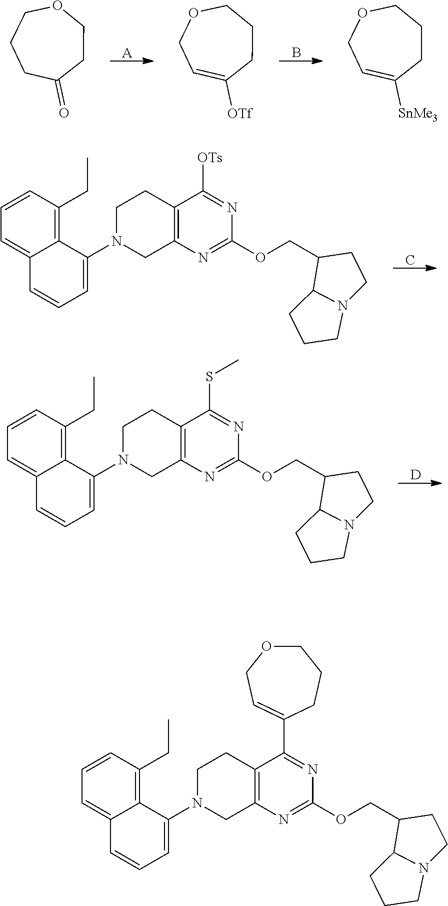

Step A. 2,5,6,7-tetrahydrooxepin-4-yl trifluoromethanesulfonate: A mixture of oxepan-4-one (3.00 g, 1.0 equiv) and 1,1,1-trifluoro-N-phenyl-N-(trifluoromethylsulfonyl)methanesulfonamide (10.3 g, 1.1 equiv) in THF (70 mL) was degassed and purged with nitrogen 3 times, then LDA (2.0 M, 15.8 mL, 1.2 equiv) was added dropwise at -65° C. The mixture was stirred at −65° C. for 0.5 hours. The mixture was quenched with ammonium chloride (aq., 100 mL) and extracted with ethyl acetate (100 mL×2). The combined organic layers were dried, filtered, concentrated, and purified by column chromatography (SiO₂, petroleum ether/ethyl acetate=10/1 to 1/1) to afford the title compound (7.2 g, crude) as yellow liquid; ¹H NMR (400 MHz, CHLOROFORM-d) δ=5.88 (t, J=6.0 Hz, 1H), 5.73 (t, J=4.4 Hz, 1H), 4.12-4.08 (m, 2H), 3.76 (t, J=6.0 Hz, 2H), 3.70 (t, J=5.2 Hz, 3H), 2.64 (t, J=5.2 Hz, 1H), 2.60-2.51 (m, 2H), 2.27 (br d, J=4.8 Hz, 1H), 1.93-1.82 (m, 2H).

Step B. trimethyl(2,5,6,7-tetrahydrooxepin-4-yl)stannane: A mixture of 2,5,6,7-tetrahydrooxepin-4-yl trifluoromethanesulfonate (4.3 g, 1.0 equiv), trimethyl(trimethylstannyl)stannane (6.9 g, 1.2 equiv), Pd(PPh3)4 (2.0 g, 0.10 equiv), and LiCi (2.2 g, 3.0 equiv) in THE (50 mL) was degassed and purged with nitrogen 3 times, and then the mixture was stirred at 60° C. for 16 hours under nitrogen atmosphere. The mixture was filtered. The filtrate was concentrated and purified with column chromatography [SiO₂, petroleum ether/ethyl acetate=I/O to 5/1] to afford the title compound (1.9 g, crude) as colorless liquid; ¹H NMR (400 MHz, CHLOROFORM-d) δ=5.97-5.86 (m, 1H), 4.28-4.20 (m, 3H), 3.94-3.88 (m, 2H), 3.69 (br dd, J=4.4, 5.6 Hz, 2H), 1.92-1.86 (m, 2H), 0.19-0.17 (m, 9H)

Step C. 7-(8-ethylnaphthalen-1-yl)-2-((hexahydro-1H-pyrrolizin-7a-yl)methoxy)-4-(methylthio)-5,6,7,8-tetrahydropyrido[3,4-d] pyrimidine: To a solution of 7-(8-ethylnaphthalen-1-yl)-2-((tetrahydro-1H-pyrrolizin-7a(5H)-yl)methoxy)-5,6,7,8-tetrahydropyrido[3,4-d]pyrimidin-4-yl 4-methylbenzenesulfonate (500 mg, 1.0 equiv) in THF (20 mL) was added NaSMe (540 mg, 9.0 equiv). The mixture was stirred at 20° C. for 0.5 hours. The organic phase was concentrated and purified with prep-HPLC [column: Unisil 3-100 C18 Ultra 150×50mm×3 μm; mobile phase: [water (FA)-ACN]; B %: 29%-59%,7 minutes] to afford the title compound (200 mg, 50% yield) as yellow solid; LCMS (ESI, M+1): m z=475.4

Step D. 7-(8-ethylnaphthalen-1-yl)-2-((hexahydro-1H-pyrrolizin-7a-yl)methoxy)-4-(2,5,6,7-tetrahydrooxepin-4-yl)-5,6,7,8-tetrahydropyrido[3,4-d] pyrimidine: A mixture of 7-(8-ethylnaphthalen-1-yl)-2-((hexahydro-1H-pyrrolizin-7a-yl)methoxy)-4-(methylthio)-5,6,7,8-tetrahydropyrido[3,4-d]pyrimidine (200 mg, 1.0 equiv), trimethyl(2,3,6,7-tetrahydrooxepin-4-yl)stannane (165 mg, 1.5 equiv), thiophene-2-carbonyloxycopper (121 mg, 1.5 equiv), (1E,4E)-1,5-diphenylpenta-1,4-dien-3-one;palladium (57.9 mg, 0.15 equiv), and tris(2-furyl)phosphane (29.4 mg, 0.30 equiv) in dioxane (5.0 mL) was degassed and purged with nitrogen 3 times, and then the mixture was stirred at 80° C. for 2 hours under nitrogen atmosphere. The organic phase was dried, filtered, concentrated, and purified prep-TLC (SiO₂, dichloromethane/methyl alcohol=10:1) and then further purified with prep-HPLC [column: Phenomenex C18 75×30 mmx 3 μm; mobile phase: [water(FA)-ACN];B %: 35%-45%,7 minutes] to give the title compound (3 mg, 1.25% yield) as yellow solid (FA salt). ¹H NMR (400 MHz, METHANOL-d₄) δ=8.56 (s, 1H), 7.72 (dd, J=7.6, 14.4 Hz, 2H), 7.48-7.41 (m, 1H), 7.40-7.34 (m, 2H), 7.32-7.27 (m, 1H), 6.03 (t, J=4.4 Hz, 1H), 4.42-4.25 (m, 4H), 4.17 (br d, J=17.6 Hz, 1H), 3.96 (t, J=6.0 Hz, 2H), 3.83 (br d, J=17.6 Hz, 1H), 3.63-3.45 (m, 2H), 3.32-3.25 (m, 3H), 3.24-3.01 (m, 2H), 2.98-2.75 (m, 4H), 2.68-2.55 (m, 1H), 2.18-1.83 (m, 10H), 1.13 (t, J=7.2 Hz, 3H) LCMS (ESI, M+1): m/z=525.4

### Example 475

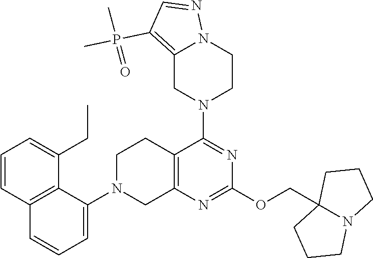 [SMILES: CCc1cccc2cccc(N3CCc4c(nc(OCC56CCCN5CCC6)nc4N4CCn5ncc(P(C)(C)=O)c5C4)C3)c12]

### (5-(7-(8-ethylnaphthalen-1-yl)-2-((tetrahydro-1H-pyrrolizin-7a(5H)-yl)methoxy)-5,6,7,8-tetrahydropyrido[3,4-d]pyrimidin-4-yl)-4,5,6,7-tetrahydropyrazolo[1,5-a]pyrazin-3-yl)dimethylphosphine oxide

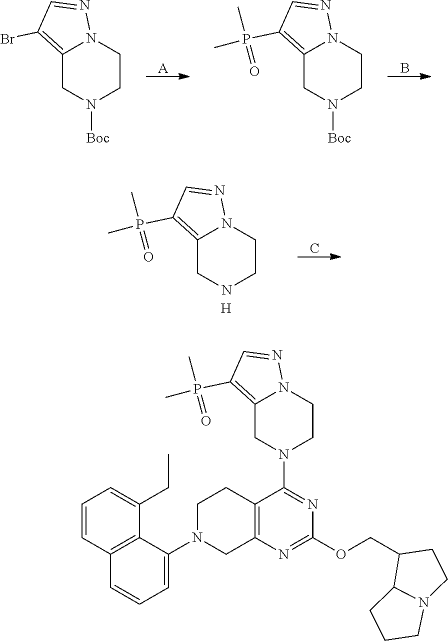

Step A. tert-butyl 3-(dimethylphosphoryl)-6,7-dihydropyrazolo[1,5-a]pyrazine-5(4H)-carboxylate: A mixture of tert-butyl 3-bromo-6,7-dihydro-4H-pyrazolo[1,5-a]pyrazine-5-carboxylate (500 mg, 1.0 equiv), methylphosphonoylmethane (155 mg, 1.2 equiv), palladium acetate (37.15 mg, 0.10 equiv), Xantphos (95.7 mg, 0.10 equiv) and potassium phosphate (386 mg, 1.1 equiv) in dimethylformamide (5 mL) was degassed and purged with N₂ 3 times, and then the mixture was stirred at 130° C. for 16 hours under N₂ atmosphere. The organic phase was separated, dried, filtered, concentrated, and purified with prep-HPLC [column: Phenomenex C18 75 * 30 mm*3 μm; mobile phase: [water (FA)-ACN]; B %: 12%-42%,7 minutes] to give the title compound (130 mg, 26% yield) as a yellow oil. LCMS (ESI, M+1): m/z=300.1

Step B. Dimethyl(4,5,6,7-tetrahydropyrazolo[1,5-a]pyrazin-3-yl)phosphine oxide: To a solution of tert-butyl 3-dimethylphosphoryl-6,7-dihydro-4H-pyrazolo[1,5-a]pyrazine-5-carboxylate (70.0 mg, 1.0 equiv) in methanol (1.0 mL) was added HCl/MeOH (8.77 mg, 1.0 equiv).The mixture was stirred at 25° C. for 2 hours. The reaction was concentrated and triturated with ethyl acetate at 25° C. for 30 minutes to give the title compound (40.0 mg, 86% yield) as a yellow oil. LCMS (ESI, M+1): m/z=200.1

Step C. (5-(7-(8-ethylnaphthalen-1-yl)-2-((tetrahydro-1H-pyrrolizin-7a(5H)-yl)methoxy)-5,6,7,8-tetrahydropyrido[3,4-d]pyrimidin-4-yl)-4,5,6,7-tetrahydropyrazolo[1,5-a]pyrazin-3-yl)dimethylphosphine oxide: To a solution of 3-dimethylphosphoryl-4,5,6,7-tetrahydropyrazolo[1,5-a]pyrazine (70.0 mg, 4.0 equiv, HCl) in dimethylformamide (0.5 mL) were added 7-(8-ethylnaphthalen-1-yl)-2-((tetrahydro-1H-pyrrolizin-7a(5H)-yl)methoxy)-5,6,7,8-tetrahydropyrido[3,4-d]pyrimidin-4-yl 4-methylbenzenesulfonate, diisopropylethylamine (28.8 mg, 3.0 equiv) and 4 Å molecular sieve (7 mg). The mixture was stirred at 25° C. for 2 hours. The organic phase was separated, dried, filtered, concentrated, and purified by reversed-phase HPLC [column: Phenomenex luna C18 150 * 25 mm*10 μm; mobile phase: [water (FA)-ACN]; B %: 18%-48%,10 minutes] to give the title compound (3.00 mg) as a yellow oil (FA salt). ¹H NMR (400 MHz, DMSO-d₆) δ=8.28 (s, 1H), 7.77 (s, 1H), 7.75-7.69 (m, 2H), 7.48 (s, 1H), 7.39 (d, J=7.2 Hz, 2H), 7.32 (s, 1H), 5.03 (s, 1H), 4.88 (s, 1H), 4.42 (br s, 1H), 4.32-4.15 (m, 2H), 4.01-3.88 (m, 3H), 3.79 (br s, 2H), 3.68 (br d, J=17.6 Hz, 2H), 3.25-3.10 (m, 4H), 3.07-2.91 (m, 4H), 2.73-2.65 (m, 2H), 2.56-2.55 (m, 1H), 2.37-2.26 (m, 1H), 2.37-2.25 (m, 1H), 1.91-1.68 (m, 6H), 1.65 (s, 3H), 1.61 (s, 3H), 1.57 (br d, J=12.0 Hz, 2H), 1.11 (t, J=7.2 Hz, 3H).

### Example 476

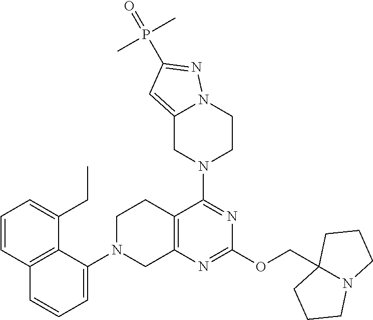 [SMILES: CCc1cccc2cccc(N3CCc4c(nc(OCC56CCCN5CCC6)nc4N4CCn5nc(P(C)(C)=O)cc5C4)C3)c12]

### (5-(7-(8-ethylnaphthalen-1-yl)-2-((tetrahydro-1H-pyrrolizin-7a(5H)-yl)methoxy)-5,6,7,8-tetrahydropyrido[3,4-d]pyrimidin-4-yl)-4,5,6,7-tetrahydropyrazolo[1,5-a]pyrazin-2-yl)dimethylphosphine oxide

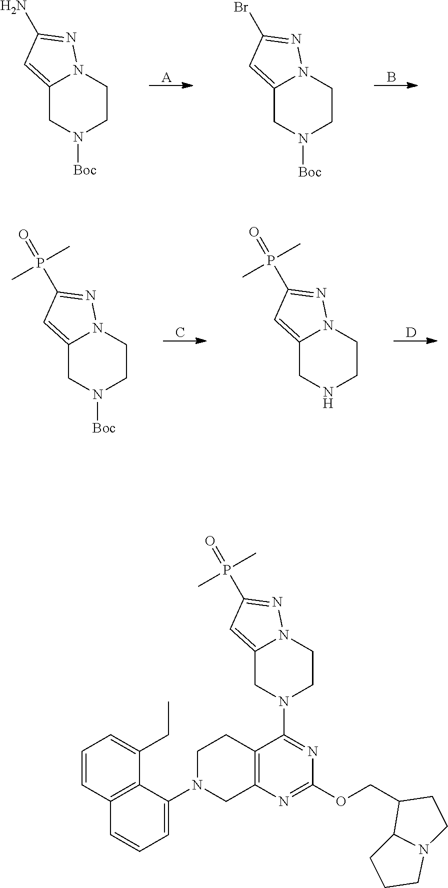

Step A. tert-butyl 2-bromo-6,7-dihydropyrazolo[1,5-a]pyrazine-5(4H)-carboxylate: To a solution of tert-butyl nitrite (454 mg, 1.5 equiv) and lithium bromide (306 mg, 1.2 equiv) in acetonitrile (10 mL) was added dropwise cuprous bromide (632.11 mg, 1.5 equiv) at 25° C. After addition, the mixture was stirred at this temperature for 10 minutes, and then tert-butyl 2-amino-6,7-dihydro-4H-pyrazolo[1,5-a]pyrazine-5-carboxylate (700 mg, 1 eq) in acetonitrile (10 mL) was added dropwise at 25° C. The resulting mixture was stirred at 50° C. for 1 hour. The reaction was extracted dried, filtered, concentrated, and purified with column chromatography [SiO₂, dichloromethane: methanol=10:1] to give the title compound (300 mg, 34% yield) was obtained as a yellow solid. ¹H NMR (400 MHz, CDCl₃) δ=6.01 (s, 1H), 4.54 (s, 2H), 4.06 (br d, J=5.4 Hz, 2H), 3.80 (br s, 2H), 1.42 (s, 9H). LCMS (ESI, M+1): m/z=302.1

Step B. tert-butyl 2-(dimethylphosphoryl)-6,7-dihydropyrazolo[1,5-a]pyrazine-5(4H1)-carboxylate: A mixture of tert-butyl 2-bromo-6,7-dihydro-4H-pyrazolo[1,5-a]pyrazine-5-carboxylate (300 mg, 1.0 equiv), methylphosphonoylmethane (77.5 mg, 1.0 equiv), palladium acetate (22.3 mg, 0.10 equiv), Xantphos (57.5 mg, 0.10 equiv), and potassium phosphate (231.82 mg, 1.1 equiv) in dimethylformamide (10 mL) was degassed and purged with N₂ 3 times, and then the mixture was stirred at 130° C. for 10 hours under N₂ atmosphere. The reaction was quenched by addition water (5 mL) at 25° C., and then diluted with water (10 mL) and extracted with ethyl acetate (20 mL). The combined organic layers were dried, filtered and concentrated to give the title compound. ¹H NMR (400 MHz, CDCl₃) δ=6.59-6.37 (m, 1H), 4.59 (s, 2H), 4.14 (br t, J=5.0 Hz, 2H), 3.83 (br t, J=5.2 Hz, 2H), 1.69 (s, 3H), 1.67-1.62 (m, 3H), 1.43 (s, 9H). LCMS (ESI, M+1): m/z=300.2

Step C: Dimethyl(4,5,6,7-tetrahydropyrazolo[1,5-a]pyrazin-2-yl)phosphine oxide: To a solution of tert-butyl 2-dimethylphosphoryl-6,7-dihydro-4H-pyrazolo[1,5-a]pyrazine-5-carboxylate (100 mg, 1.0 equiv) in methanol (2.0 mL) was added HCl/MeOH (4 M, 83.53 pL, 1 eq) and the mixture was stirred at 25° C. for 1 hour. The reaction was concentrated and triturated with petroleum ether at 25° C. for 10 minutes to give the title compound (70.0 mg, 89% yield, HCl) as a yellow oil. LCMS (ESI, M+1): m/z=200.2

Step D. (5-(7-(8-ethylnaphthalen-1-yl)-2-((tetrahydro-1H-pyrrolizin-7a(5H)-yl)methoxy)-5,6,7,8-tetrahydropyrido[3,4-d]pyrimidin-4-yl)-4,5,6,7-tetrahydropyrazolo[1,5-a]pyrazin-2-yl)dimethylphosphine oxide: To a solution of 7-(8-ethylnaphthalen-1-yl)-2-((tetrahydro-1H-pyrrolizin-7a(5H)-yl)methoxy)-5,6,7,8-tetrahydropyrido[3,4-d]pyrimidin-4-yl 4-methylbenzenesulfonate (45.8 mg, 1.0 equiv) in dimethylformamide (0.50 mL) and acetonitrile (0.5 mL) was added N,N-diethylpropan-2-amine (28.8 mg, 3.0 equiv) and 2-dimethylphosphoryl-4,5,6,7-tetrahydropyrazolo[1,5-a]pyrazine (70.0 mg, 4.0 equiv, HCl). The mixture was stirred at 40° C. for 2 hours. The reaction was quenched by addition of water (5 mL) at 25° C., and then diluted further with water (10 mL) and extracted, dried, filtered, concentrated, and purified with prep-HPLC [column: Phenomenex luna C18 150 * 25 mm*10 μm; mobile phase: [water(FA)-ACN]; B %: 18%-48%,10 minutes] to give the title compound (2.2 mg) as a white solid (FA salt). ¹H NMR (400 MHz, DMSO-d₆) δ=7.80-7.76 (m, 1H), 7.73 (br d, J=8.0 Hz, 1H), 7.50-7.45 (m, 1H), 7.38 (br dd, J=8.0, 10.4 Hz, 2H), 7.32 (br d, J=6.8 Hz, 1H), 6.64 (s, 1H), 4.82 (s, 1H), 4.77-4.70 (m, 1H), 4.45 (br s, 2H), 4.34 (br s, 2H), 4.19 (br d, J=14.4 Hz, 2H), 3.97 (s, 3H), 3.76-3.66 (m, 4H), 3.20 (br d, J=9.2 Hz, 3H), 3.08-2.98 (m, 4H), 2.29 (s, 1H), 1.93-1.73 (m, 6H), 1.66 (s, 3H), 1.62 (s, 3H), 1.61-1.55 (m, 1H), 1.12 (t, J=7.2 Hz, 2H). LCMS (ESI, M+1): m/z=626.3.

### Example 477

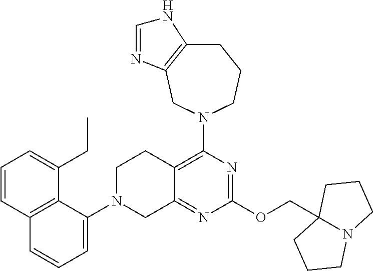 [SMILES: CCc1cccc2cccc(N3CCc4c(nc(OCC56CCCN5CCC6)nc4N4CCCc5[nH]cnc5C4)C3)c12]

### 5-(7-(8-ethylnaphthalen-1-yl)-2-((hexahydro-1H-pyrrolizin-7a-yl)methoxy)-5,6,7,8-tetrahydropyrido[3,4-d]pyrimidin-4-yl)-1,4,5,6,7,8-hexahydroimidazo[4,5-c]azepine

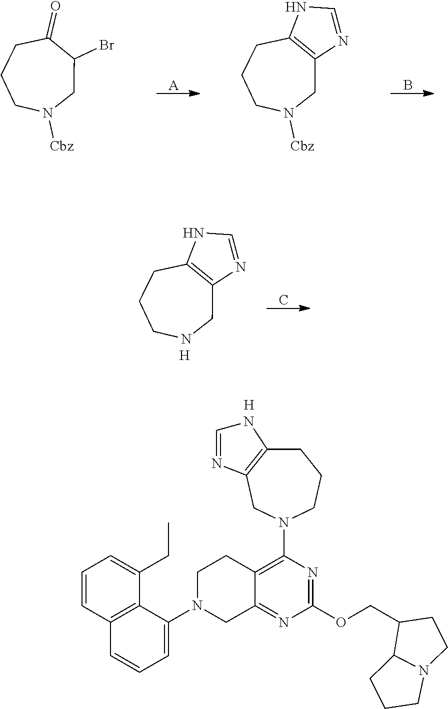

Step A. benzyl 4,6,7,8-tetrahydroimidazo[4,5-c]azepine-5(1H)-carboxylate: To a mixture of benzyl 3-bromo-4-oxoazepane-1-carboxylate (1.00 g, 1.0 equiv) in t-BuOH (10 mL) was added formimidamide (958 mg, 3.0 equiv)). The reaction was stirred at 70° C. for 12 hours. The mixture was concentrated and purified by prep-HPLC (column: Unisil 3-100 C18 Ultra 150 x 50 mm×3 m; mobile phase: [water (FA) -CAN]; B %: 5%-40%, 27 min) to afford the title compound (143 mg, 16% yield) as a yellow oil. LCMS (ESI, M+1): m/z=271.9.

Step B. 1,4,5,6,7,8-hexahydroimidazo[4,5-c]azepine: To a mixture of benzyl 4,6,7,8-tetrahydroimidazo[4,5-c]azepine-5(1H)-carboxylate (100 mg, 1.0 equiv) in MeOH (2.0 mL) was added Pd/C (400 mg, 10% purity). The mixture was degassed and purged with nitrogen 3 times. The reaction was stirred at 25° C. for 1 hour under H2 at 15 psi. The mixture was filtered and concentrated under vacuum to afford the title compound (80 mg, crude) as white oil.

Step C. 5-(7-(8-ethylnaphthalen-1-yl)-2-((hexahydro-1H-pyrrolizin-7a-yl)methoxy)-5,6,7,8-tetrahydropyrido[3,4-d]pyrimidin-4-yl)-1,4,5,6,7,8-hexahydroimidazo[4,5-c]azepine: To a mixture of 7-(8-ethylnaphthalen-1-yl)-2-((hexahydro-1H-pyrrolizin-7a-yl)methoxy)-5,6,7,8-tetrahydropyrido[3,4-d]pyrimidin-4-yl 4-methylbenzenesulfonate (142 mg, 1.0 equiv) and 1,4,5,6,7,8-hexahydroimidazo[4,5-c]azepine (65.0 mg, 2.0 equiv) in DMF (1.0 mL) were added N,N-diethylpropan-2-amine (306 mg, 10 equiv) and 4 Å molecular sieve (10.0 mg, 1.0 equiv). The reaction was stirred at 40° C. for 12 hours. The mixture was concentrated and purified by prep-HPLC (column: Phenomenex luna C18 150×25 mm×10 μm; mobile phase: [water (FA)-ACN]; B %: 12%-42%, 10 min) to afford the title compound (29 mg) as yellow solid (FA salt). ¹H NMR (400 MHz, DMSO-d₆) δ=7.73-7.64 (m, 2H), 7.62 (s, 1H), 7.42 (t, J=7.6 Hz, 1H), 7.37-7.25 (m, 3H), 5.01 (br d, J=15.6 Hz, 1H), 4.63 (br d, J=15.6 Hz, 1H), 4.40-4.26 (m, 3H), 4.04 (br d, J=17.6 Hz, 1H), 3.88 (br dd, J=8.4, 15.2 Hz, 1H), 3.70-3.51 (m, 5H), 3.27-3.17 (m, 4H), 3.10-2.99 (m, 1H), 2.93-2.84 (m, 1H), 2.83-2.71 (m, 2H), 2.33-2.22 (m, 2H), 2.18 (td, J=6.4, 12.8 Hz, 2H), 2.13-1.97 (m, 5H), 1.91-1.77 (m, 1H), 1.14 (t, J=7.2 Hz, 3H). LCMS (ESI, M+1): m/z=564.5.

### Example 478

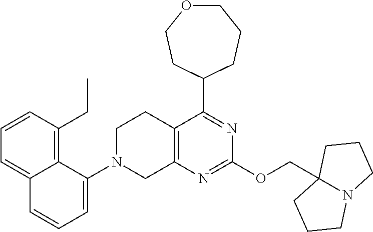 [SMILES: CCc1cccc2cccc(N3CCc4c(nc(OCC56CCCN5CCC6)nc4C4CCCOCC4)C3)c12]

### 7-(8-ethylnaphthalen-1-yl)-2-((hexahydro-1H-pyrrolizin-7a-yl)methoxy)-4-(oxepan-4-yl)-5,6,7,8-tetrahydropyrido[3,4-d]pyrimidine

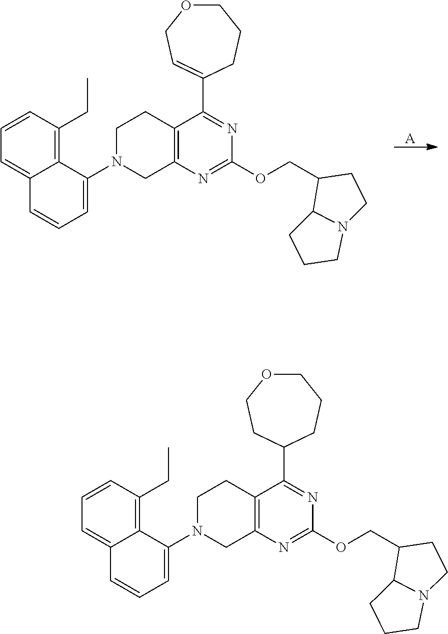

Step A. 7-(8-ethylnaphthalen-1-yl)-2-((hexahydro-1H-pyrrolizin-7a-yl)methoxy)-4-(oxepan-4-yl)-5,6,7,8-tetrahydropyrido[3,4-d]pyrimidine: A mixture of 7-(8-ethylnaphthalen-1-yl)-2-((tetrahydro-1H-pyrrolizin-7a(5H)-yl)methoxy)-4-(2,3, 6,7-tetrahydrooxepin-4-yl)-5,6,7,8-tetrahydropyrido[3,4-d]pyrimidine (15.0 mg, 1.0 equiv) and Pd/C (14.3 mg, 10% purity, 1.0 equiv) in ethyl acetate (1.0 mL) was degassed and purged with hydrogen 3 times, and then the mixture was stirred at 20° C. for 0.1 hour under hydrogen atmosphere. The mixture was diluted with ethyl acetate (20 mL) and filtered. The filtrate was concentrated and purified by prep-TLC (SiO₂, dichloromethane/methyl alcohol=10:1) and then further purified by prep-HPLC (column: Phenomenex C18 75×30 mm×3 μm; mobile phase: [water(FA)-ACN];B %: 25%-55%,7 min).to afford the title compound (3 mg, 20% yield) as white solid; ¹H NMR (400 MHz, METHANOL-d₄) δ=7.76-7.65 (m, 2H), 7.47-7.40 (m, 1H), 7.35 (dd, J=7.3, 9.5 Hz, 2H), 7.31-7.24 (m, 1H), 4.30-4.20 (m, 2H), 4.08 (br d, J=17.2 Hz, 1H), 4.01-3.87 (m, 2H), 3.86-3.68 (m, 4H), 3.62 (br dd, J=6.0, 12.0 Hz, 1H), 3.46-3.35 (m, 2H), 3.19 (br dd, J=5.6, 11.2 Hz, 2H), 3.14-3.01 (m, 2H), 2.92 (br d, J=15.6 Hz, 1H), 2.86-2.78 (m, 2H), 2.22-2.04 (m, 4H), 2.00-1.88 (m, 8H), 1.85-1.74 (m, 2H), 1.12-1.04 (m, 3H); LCMS (ESI, M+1): m/z=527.2

### Example 479

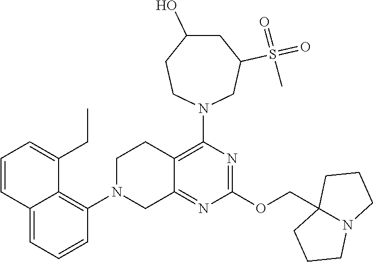 [SMILES: CCc1cccc2cccc(N3CCc4c(nc(OCC56CCCN5CCC6)nc4N4CCC(O)CC(S(C)(=O)=O)C4)C3)c12]

### 1-(7-(8-ethylnaphthalen-1-yl)-2-((tetrahydro-1H-pyrrolizin-7a(5H)-yl)methoxy)-5,6,7,8-tetrahydropyrido[3,4-d]pyrimidin-4-yl)-6-(methylsulfonyl)azepan-4-ol (isomer 1)

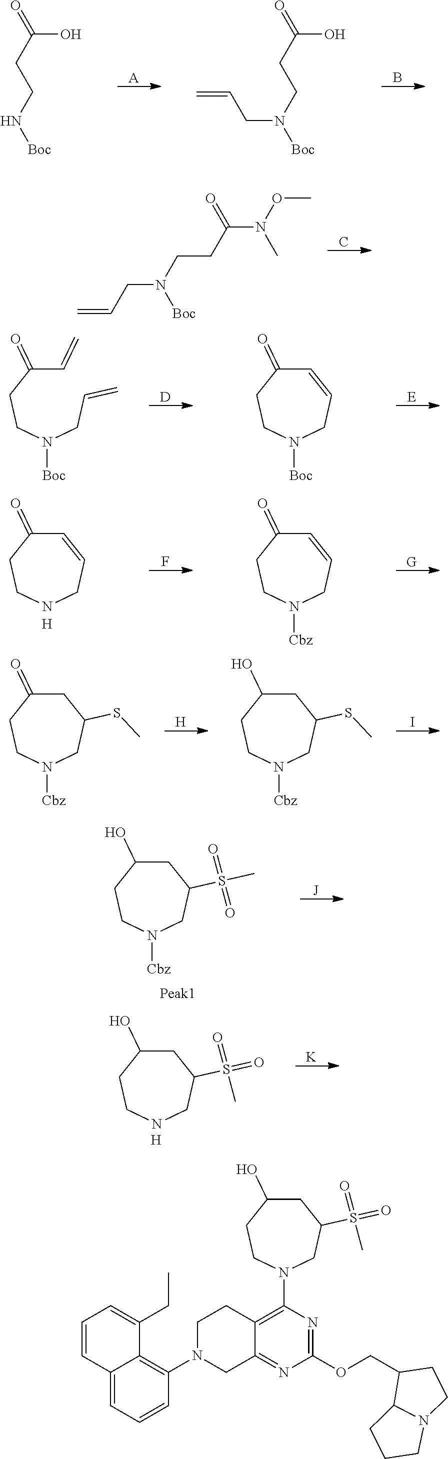

Step A. 3-(allyl(tert-butoxycarbonyl)amino)propanoic acid: To a solution of 3-(tert-butoxycarbonylamino)propanoic acid (25.0 g, 1.0 equiv) in THE (500 mL) was added NaH (26.4 g, 60% purity, 5.0 equiv) at 0° C. After stirring for 1 hour, 3-bromoprop-1-ene (48.0 g, 3.0 equiv) was added to the mixture dropwise. The reaction was stirred at 25° C. for 12 hours. The mixture was cooled to 0° C., quenched with H20, and washed with hexanes (2×50 mL). The aqueous layer was acidified with 1 M HCl aqueous solution until pH=3 and then the solution was extracted with EtOAc (3×75 mL). The combined organic layers were dried, filtered, and concentrated to afford the title compound (30 g, 99.0% yield) as yellow oil which was used into the next step directly without further purification; ¹H NMR (400 MHz, CDCl₃) δ=5.87-5.68 (m, 1H), 5.14 (br d, J=10.8 Hz, 2H), 3.86 (br s, 2H), 3.49 (br d, J=6.0 Hz, 2H), 2.63 (br s, 2H), 1.46 (s, 9H).

Step B. tert-butyl allyl(3-(methoxy(methyl)amino)-3-oxopropyl)carbamate: To a mixture of 3-(allyl(tert-butoxycarbonyl)amino)propanoic acid (30.0 g, 1.0 equiv), TEA (59.6 g, 4.5 equiv), and EDCI (30.1 g, 1.2 equiv) in DCM (1.5 L) were added N-methoxymethanamine (25.5 g, 2 equiv, HCl) and DMAP (17.6 g, 1.1 equiv). The reaction was stirred at 25° C. for 12 hours. The mixture was diluted with water (500 mL) and extracted with DCM (1 L×3). The combined organic phases were washed with citric acid (500 mL), saturated NaHCO₃ (500 mL), brine (500 mL), dried, concentrated, and purified by column chromatography (SiO2, Petroleum ether/Ethyl acetate=10/1 to 3/1) to afford the title compound (23.0 g, 65% yield) as yellow oil; ¹H NMR (400 MHz, CDCl₃) δ=5.83-5.67 (m, 1H), 5.09 (br d, J=8.1 Hz, 2H), 3.84 (br s, 2H), 3.66 (s, 3H), 3.47 (br s, 2H), 3.15 (s, 3H), 2.67 (br s, 2H), 1.51-1.32 (m, 9H).

Step C. tert-butyl allyl(3-oxopent-4-en-1-yl)carbamate: To a solution of tert-butyl allyl(3-(methoxy(methyl)amino)-3-oxopropyl)carbamate (19.0 g, 1.0 equiv) in THF (160 mL) was added bromo(vinyl)magnesium (1 M, 209 mL, 3.0 equiv). The reaction was stirred at 25° C. for 1 hour. The mixture was quenched with water (10.0 mL) and extracted with ethyl acetate (20.0 mL×3). The combined organic layers were washed with brine (20.0 mL), dried, concentrated, and purified by column chromatography (SiO2, Petroleum ether/Ethyl acetate=50/1 to 10/1) to afford the title compound (3.7 g, 22.2% yield) as yellow oil; ¹H NMR (400 MHz, CDCl₃) δ=6.47-6.17 (m, 2H), 5.88 (br d, J=10.2 Hz, 1H), 5.83-5.70 (m, 1H), 5.12 (br d, J=10.8 Hz, 2H), 3.91-3.76 (m, 2H), 3.48 (br d, J=6.0 Hz, 2H), 2.89 (br s, 2H), 1.45 (s, 9H).

Step D. tert-butyl 4-oxo-2,3,4,7-tetrahydro-1H-azepine-1-carboxylate: A mixture of tert-butyl allyl(3-oxopent-4-en-1-yl)carbamate (3.70 g, 1.0 equiv) and Ti(Oi-Pr)₄ (879 mg, 0.2 equiv) in DCM (370 mL) was added Ru(CHPh)Cl₂(1,3-dimesityl-4,5-dihydroimidazol-2-ylidene)(PCy₃) (656 mg, 0.05 equiv). The mixture was degassed and purged with N₂ 3 times. The reaction was stirred at 40° C. for 14 hours. The mixture was concentrated and purified by flash silica gel chromatography (ISCO®; 40 g SepaFlash® Silica Flash Column, Eluent of 0˜30% Ethyl acetate/Petroleum ether gradient @100 mL/min) to afford the title compound (2.5 g, 92.7% purity) as yellow oil; ¹H NMR (400 MHz, CHLOROFORM-d) δ=6.47-6.30 (m, 1H), 6.04 (br d, J=12.6 Hz, 1H), 4.44-4.28 (m, 2H), 3.58 (br d, J=4.9 Hz, 2H), 2.82 (br d, J=5.3 Hz, 2H), 1.48 (s, 9H); LCMS (ESI, M-55): m/z=156.0.

Step E. 1,2,3,7-tetrahydro-4H-azepin-4-one: To a solution of tert-butyl 4-oxo-2,3,4,7-tetrahydro-1H-azepine-1-carboxylate (2.40 g, 1.0 equiv) in ACN (6 mL) was added HCl•dioxane (4 M, 2.84 mL). The reaction was stirred at 0° C. for 1 hour. The mixture was concentrated at 25° C. to afford the title compound (1.26 g, crude) as brown solid.

Step F. benzyl 4-oxo-2,3,4,7-tetrahydro-1H-azepine-1-carboxylate: To a solution of 1,2,3,7-tetrahydro-4H-azepin-4-one (1.26 g, 1.0 equiv) in THE (20 mL) were added TEA (3.44 g, 3.0 equiv) and CbzCl (2.90 g, 1.5 equiv). The reaction was stirred at 25° C. for 12 hours. The mixture was diluted with water (12.0 mL) and extracted with ethyl acetate (12.0 mL×3). The combined organic layers were washed with brine (12.0 mL), dried, concentrated, and purified by flash silica gel chromatography (ISCO®; 20 g SepaFlash® Silica Flash Column, Eluent of 0˜30% Ethylacetate/Petroleum ether gradient @75 mL/min) to afford the title compound (0.99 g, 35.6% yield) as brown oil; ¹H NMR (400 MHz, CDCl₃) δ=7.51-7.32 (m, 5H), 6.44-6.27 (m, 1H), 6.11-6.02 (m, 1H), 5.25-5.09 (m, 2H), 4.49-4.34 (m, 2H), 3.74-3.59 (m, 2H), 2.93-2.75 (m, 2H).

Step G. benzyl 3-(methylthio)-5-oxoazepane-1-carboxylate: To a solution of benzyl 4-oxo-2,3,4,7-tetrahydro-1H-azepine-1-carboxylate (490 mg, 1.0 equiv) in toluene (6 mL) were added NaSMe (1.40 g, 20% in water, 2.0 equiv) and AcOH (239.93 mg, 2 equiv). The mixture was stirred at 40° C. for 12 hours. Additional NaSMe (1.40 g, 20% in water, 2.0 equiv) was added to the mixture. The reaction was stirred at 40° C. for 2 hours. The mixture was diluted with water (2.00 mL) and extracted with ethyl acetate (2.00 mL×3). The combined organic phases were washed with brine (2.0 mL), dried, concentrated, and purified by prep-TLC (PE:EA=3:1) to afford the title compound (305 mg, 52.04% yield) as colorless oil; ¹H NMR (400 MHz, CDCl₃) δ=7.28 (br s, 5H), 5.16-4.99 (m, 2H), 4.21-3.94 (m, 1H), 3.88-3.72 (m, 1H), 3.52-3.40 (m, 1H), 3.38-3.23 (m, 1H), 2.96-2.72 (m, 3H), 2.70-2.49 (m, 2H), 2.19-1.92 (m, 3H); LCMS (ESI, M+1): m/z=294.0.

Step H. benzyl 5-hydroxy-3-(methylthio)azepane-1-carboxylate: To a solution of benzyl 3-methylsulfanyl-5-oxo-azepane-1-carboxylate (300 mg, 1 equiv) in MeOH (5 mL) was added NaBH4 (77.4 mg, 2 equiv) at 0 C. The reaction was stirred at 20° C. for 2 hours. The mixture was quenched with NH₄Cl (2.00 mL) and extracted with ethyl acetate (2.00 mL×3). The combined organic layers were washed with brine (2.0 mL), dried, and concentrated to afford the title compound (300 mg, crude) as colorless oil, which was used into next step directly without further purification; ¹H NMR (400 MHz, CDCl₃) δ=7.52-7.28 (m, 5H), 5.15 (br s, 2H), 4.26-3.98 (m, 1H), 3.96-3.83 (m, 1H), 3.79-3.55 (m, 1H), 3.43-2.65 (m, 3H), 2.32-1.95 (m, 5H), 1.93-1.72 (m, 2H); LCMS (ESI, M+1): m/z=296.0.

Step I. benzyl 5-hydroxy-3-(methylsulfonyl)azepane-1-carboxylate: To a solution of oxone (1.25 g, 2.0 equiv) in MeOH (5 mL) was added a solution of benzyl 5-hydroxy-3-methylsulfanylazepane-1-carboxylate (300 mg, 1.0 equiv) in H₂O (5 mL). The reaction was stirred at 0° C. for 1 hour. The mixture was quenched with Na₂SO₃ (10.0 mL) and extracted with ethyl acetate (10.0 mL×3). The combined organic layers were washed with brine (10.0 mL), dried, and concentrated to afford benzyl 5-hydroxy-3-(methylsulfonyl)azepane-1-carboxylate (0.6 g, 91.2 purity). The residue was then separated by SFC {column: DAICEL CHIRALPAK AD (250 mm×30 mm,10 um); mobile phase: [0.1% NH₃H₂O ETOH]; B %: 30%-30%,10.1 min} to afford 5-hydroxy-3-methylsulfonyl-azepane-1-carboxylate (peak 1) (240 mg) as colorless oil and benzyl 5-hydroxy-3-methylsulfonyl-azepane-1-carboxylate (peak 2) (360 mg) as a colorless oil.

Peak1: ¹H NMR (400 MHz, CDCl₃) δ=7.40-7.22 (m, 5H), 5.18-5.02 (m, 2H), 4.10-3.85 (m, 1H), 3.83 (br d, J=8.2 Hz, 1H), 3.78-3.41 (m, 3H), 3.39-3.02 (m, 1H), 2.99-2.55 (m, 3H), 2.43-2.17 (m, 1H), 2.10-1.79 (m, 3H); SFC: “Column: Chiralpak AD-3 50×4.6 mm I.D., 3 μm; Mobile phase: Phase A for CO₂, and Phase B for EtOH(0.05% DEA); Gradient elution:EtOH (0.05% DEA) in CO₂ from 5% to 40%; Flow rate: 3 mL/min;Detector: PDA Column Temp: 35C;Back Pressure: 100Bar”; LCMS (ESI, M+1): m/z=328.2.

Peak 2: ¹H NMR (400 MHz, CDCl₃) δ=7.42-7.21 (m, 5H), 5.14-5.02 (m, 2H), 4.13-3.87 (m, 1H), 3.86-3.79 (m, 1H), 3.76-3.34 (m, 4H), 2.97-2.84 (m, 2H), 2.68-2.56 (m, 1H), 2.46-2.15 (m, 1H), 1.92-1.80 (m, 3H); SFC: “Column: Chiralpak AD-3 50×4.6 mm I.D., 3 μm; Mobile phase: Phase A for CO₂, and Phase B for EtOH(0.05% DEA); Gradient elution:EtOH (0.05% DEA) in CO₂ from 5% to 40%; Flow rate: 3 mL/min;Detector: PDA Column Temp: 35C;Back Pressure: 100Bar”; LCMS (ESI, M+1): m/z=328.2.

Step J. 6-(methylsulfonyl)azepan-4-ol: To a solution of benzyl 5-hydroxy-3-(methylsulfonyl)azepane-1-carboxylate (120 mg, 1.0 equiv) in MeOH (3 mL) was added Pd/C (10%, 12.0 mg) under N₂ atmosphere. The suspension was degassed and purged with H2 3 times. The reaction was stirred under H₂ (15 Psi) at 20° C. for 2 hours. The mixture was filtered and concentrated to afford the title compound (70.0 mg, 99% yield) as a colorless oil, which was used into next step without further purification.

Step K. 1-(7-(8-ethylnaphthalen-1-yl)-2-((tetrahydro-1H-pyrrolizin-7a(5H)-yl)methoxy)-5,6,7,8-tetrahydropyrido[3,4-d]pyrimidin-4-yl)-6-(methylsulfonyl)azepan-4-ol: To a mixture of 7-(8-ethylnaphthalen-1-yl)-2-((tetrahydro-1H-pyrrolizin-7a(5H)-yl)methoxy)-5,6,7,8-tetrahydropyrido[3,4-d]pyrimidin-4-yl 4-methylbenzenesulfonate (217 mg, 1.0 equiv) and N,N-diethylpropan-2-amine (140 mg, 3.0 equiv) in DMF (2 mL) were added 6-(methylsulfonyl)azepan-4-ol (peak 1, 70.0 mg, 1.0 equiv) and 4 Å molecular sieve (100 mg). The reaction was stirred at 40° C. for 12 hours. The mixture was filtered and purified with HPLC to afford the title compound (72.8 mg, 31% yield) as white solid; 1H NMR (400 MHz, METHANOL-d₄) δ=7.73-7.63 (m, 2H), 7.42 (tt, J=2.3, 7.7 Hz, 1H), 7.37-7.24 (m, 3H), 4.56-4.28 (m, 2H), 4.27-4.01 (m, 4H), 3.98-3.76 (m, 3H), 3.74-3.60 (m, 2H), 3.59-3.45 (m, 2H), 3.26-3.16 (m, 1H), 3.13-2.96 (m, 6H), 2.84-2.66 (m, 3H), 2.49-2.10 (m, 2H), 2.07-1.95 (m, 3H), 1.93-1.77 (m, 5H), 1.75-1.63 (m, 2H), 1.20-1.10 (m, 3H); SFC: “Column: Chiralpak AD-3 50×4.6 mm I.D., 3 μm Mobile phase: Phase A for CO₂, and Phase B for EtOH(0.05% DEA); Gradient elution: 40% EtOH (0.05% DEA) in CO₂; Flow rate: 3 mL/min;Detector: PDA Column Temp: 35C;Back Pressure: 100Bar”; LCMS (ESI, M+1): m/z=620.3.

### Example 480

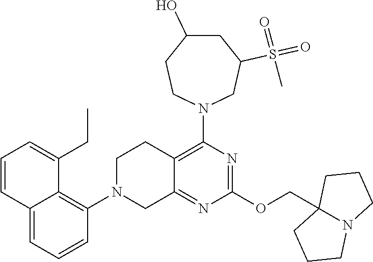 [SMILES: CCc1cccc2cccc(N3CCc4c(nc(OCC56CCCN5CCC6)nc4N4CCC(O)CC(S(C)(=O)=O)C4)C3)c12]

### 1-(7-(8-ethylnaphthalen-1-yl)-2-((tetrahydro-1H-pyrrolizin-7a(5H)-yl)methoxy)-5,6,7,8-tetrahydropyrido[3,4-d]pyrimidin-4-yl)-6-(methylsulfonyl)azepan-4-ol (isomer 2)

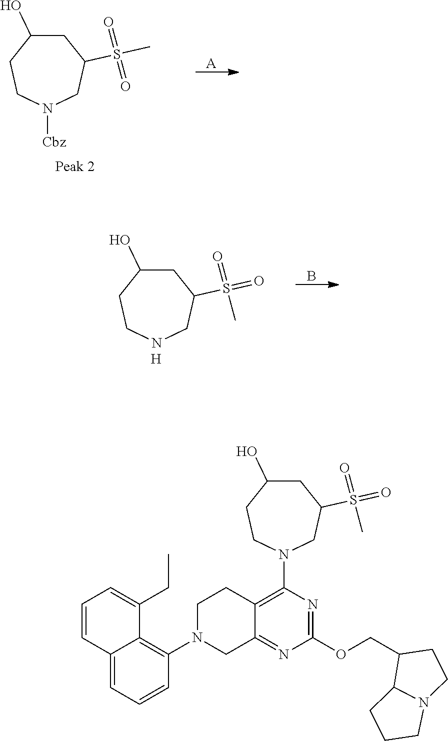

Step A. 6-(methylsulfonyl)azepan-4-ol: To a solution of benzyl 5-hydroxy-3-(methylsulfonyl)azepane-1-carboxylate (Peak 2 from step I in Example 479, 120 mg 1.0 equiv) in MeOH (2 mL) was added Pd/C (10%, 5 mg) under N₂ atmosphere. The suspension was degassed and purged with H₂ 3 times. The reaction was stirred under H₂ (15 Psi) at 20° C. for 2 hours. The mixture was filtered and concentrated to afford the title compound (70 mg, crude) as colorless oil which was used into next step directly without further purification.

Step B. 1-(7-(8-ethylnaphthalen-1-yl)-2-((tetrahydro-1H-pyrrolizin-7a(5H)-yl)methoxy)-5,6,7,8-tetrahydropyrido[3,4-d] pyrimidin-4-yl)-6-(methylsulfonyl)azepan-4-ol: To a mixture of 7-(8-ethylnaphthalen-1-yl)-2-((tetrahydro-1H-pyrrolizin-7a(5H)-yl)methoxy)-5,6,7,8-tetrahydropyrido[3,4-d]pyrimidin-4-yl 4-methylbenzenesulfonate (200 mg, 1.0 equiv) and N,N-diethylpropan-2-amine (130 mg, 3 equiv) in DMF (2 mL) were added 6-(methylsulfonyl)azepan-4-ol (67.8 mg, 1.1 equiv) and 4 Å molecular sieve (100 mg). The reaction was stirred at 40° C. for 12 hours. The mixture was filtered and purified with HPLC to afford the title compound (82.5 mg, 37% yield) as off-white solid (FA salt). ¹H NMR (400 MHz, METHANOL-d₄) δ=8.54 (s, 1H), 7.70 (dd, J=7.8, 15.2 Hz, 2H), 7.47-7.40 (m, 1H), 7.38-7.31 (m, 2H), 7.30-7.18 (m, 11H), 4.67-4.22 (m, 4H), 4.21-4.08 (m, 1H), 4.07-3.83 (m, 3H), 3.82-3.64 (m, 2H), 3.62-3.47 (m, 4H), 3.25-3.13 (m, 3H), 3.12-2.97 (m, 4H), 2.88-2.70 (m, 1H), 2.53-1.57 (m, 13H), 1.25-1.09 (m, 3H); SFC: “Column: Chiralpak AD-3 50×4.6 mm I.D., 3 μm Mobile phase: Phase A for CO₂, and Phase B for EtOH(0.05% DEA); Gradient elution: 40% EtOH (0.05% DEA) in CO₂; Flow rate: 3 mL/min;Detector: PDA Column Temp: 35C;Back Pressure: 100Bar”; LCMS (ESI, M+1): m/z=620.3.

### Example 481

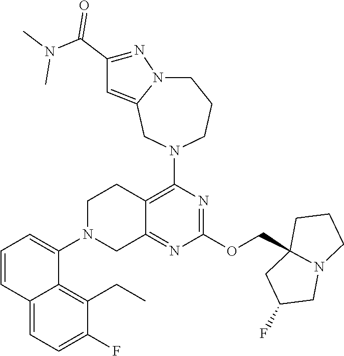 [SMILES: CCc1c(F)ccc2cccc(N3CCc4c(nc(OC[C@@]56CCCN5C[C@H](F)C6)nc4N4CCCn5nc(C(=O)N(C)C)cc5C4)C3)c12]

### 5-(7-(8-ethyl-7-fluoronaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide

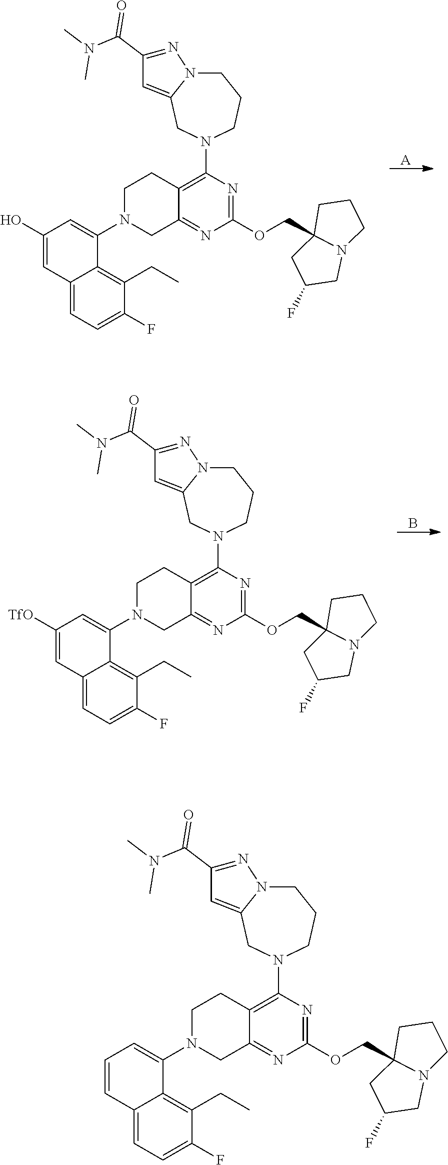

Step A. 4-(4-(2-(dimethylcarbamoyl)-7,8-dihydro-4H-pyrazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-yl trifluoromethanesulfonate: To a solution of 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide (200 mg, 1.0 equiv) and N-phenyl-O-((trifluoromethyl)sulfonyl)-N-(((trifluoromethyl)sulfonyl)oxy)hydroxylamine (156 mg, 1.5 equiv) in THE (4.0 mL) was added LiHDMS (1 M, 437 uL) at −60° C. The mixture was stirred at −60° C. for 0.5 hours. The mixture was quenched with saturated NH₄Cl aqueous solution (2 mL) and extracted with EtOAc (3×5 mL). The combined organic layers were dried over anhydrous sodium sulfate and concentrated. Then the residue was purified by silica gel chromatography [SiO2, DCM/MeOH=50/1 to 10/1] to afford the title compound (55.0 mg, 23% yield) as white solid; LCMS (ESI, M+1): m/z=819.2.

Step B. 5-(7-(8-ethyl-7-fluoronaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide: To a mixture of Pd/C (50.0 mg, 10% purity, 1.0 equiv) in EtOH (10 mL) was added 4-(4-(2-(dimethylcarbamoyl)-7,8-dihydro-4H-pyrazolo[1,5-a][1,4]diazepin-5(6H)-yl)-2-(((2R,7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-yl trifluoromethanesulfonate (55.0 mg, 1.0 equiv). The mixture was degassed and purged with H₂ 3 times and stirred at 60° C. for 12 hours. The reaction was filtered, concentrated and purified with prep-HPLC [Phenomenex Synergi C18 75×30 mm×3 μm; A: water (10 mM FA), B: ACN, B %: 20%-50% over 10 min] to afford the title compound (8.23 mg, 18% yield) as white solid. ¹H NMR (400 MHz, METHANOL-d₄) δ=8.53 (s, 1H), 7.76 (dd, J=6.0, 8.8 Hz, 1H), 7.69 (dd, J=1.6, 7.6 Hz, 1H), 7.45-7.36 (m, 2H), 7.30-7.21 (m, 1H), 6.60 (d, J=1.6 Hz, 1H), 5.41-5.21 (m, 1H), 5.03-4.96 (m, 1H), 4.83 (br d, J=2.4 Hz, 1H), 4.60-4.48 (m, 2H), 4.15 (br dd, J=4.0, 10.8 Hz, 2H), 4.11-4.00 (m, 3H), 3.70 (d, J=17.6 Hz, 1H), 3.59-3.42 (m, 3H), 3.33 (s, 4H), 3.29-3.20 (m, 4H), 3.08 (s, 3H), 3.06-3.00 (m, 1H), 2.74 (br d, J=12.0 Hz, 1H), 2.37-2.22 (m, 2H), 2.22-2.06 (m, 3H), 2.04-1.94 (m, 2H), 1.93-1.82 (m, 1H), 1.17-1.09 (m, 3H); LCMS (ESI, M+1): m/z=671.2.

### Example 482

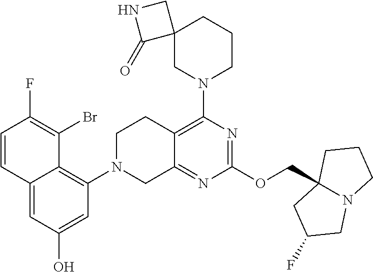 [SMILES: O=C1NCC12CCCN(c1nc(OC[C@@]34CCCN3C[C@H](F)C4)nc3c1CCN(c1cc(O)cc4ccc(F)c(Br)c14)C3)C2]

### 6-(7-(8-bromo-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-2,6-diazaspiro[3.5]nonan-1-one

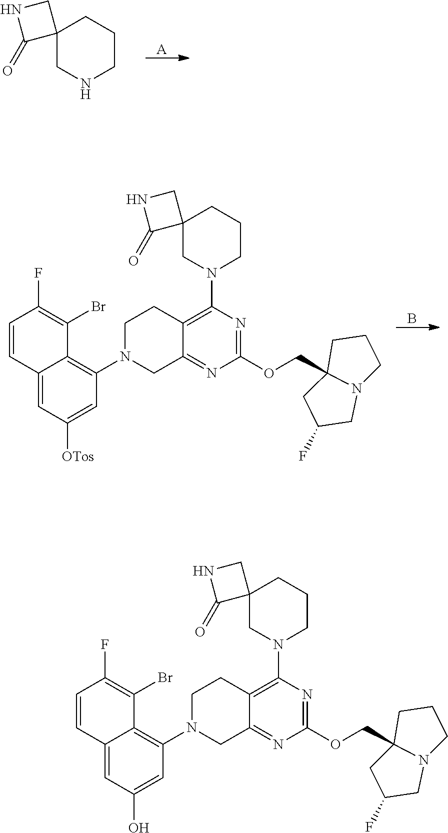

Step A. 5-bromo-6-fluoro-4-(2-(((2R,7aS)-2-fluorohexahydro-1H-pyrrolizin-7a-yl)methoxy)-4-(1-oxo-2,6-diazaspiro[3.5]nonan-6-yl)-5,6-dihydropyrido[3,4-d]pyrimidin-7(8H)-yl)naphthalen-2-yl 4-methylbenzenesulfonate: To a mixture of 5-bromo-6-fluoro-4-(2-(((2R,7aS)-2-fluorohexahydro-1H-pyrrolizin-7a-yl)methoxy)-4-(tosyloxy)-5,6-dihydropyrido[3,4-d]pyrimidin-7(8H)-yl)naphthalen-2-yl 4-methylbenzenesulfonate (40.0 mg, 1.0 equiv), 4 Å molecular sieve (5.0 mg) and N,N-diethylpropan-2-amine (121 mg, 20 equiv) in DMF (1 mL) was added 2,6-diazaspiro[3.5]nonan-1-one (29.7 mg, 2.5 equiv, TFA). The reaction was stirred at 40° C. for 12 hours. The mixture was filtered and purified by reversed phase HPLC (0.1% FA condition) to afford the title compound (30 mg, 77% yield) as yellow solid; LCMS (ESI, M+1): m/z=823.0 825.0.

Step B. 6-(7-(8-bromo-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-2,6-diazaspiro[3.5]nonan-1-one: A mixture of 5-bromo-6-fluoro-4-(2-(((2R,7aS)-2-fluorohexahydro-1H-pyrrolizin-7a-yl)methoxy)-4-(1-oxo-2,6-diazaspiro[3.5]nonan-6-yl)-5,6-dihydropyrido[3,4-d]pyrimidin-7(8H)-yl)naphthalen-2-yl 4-methylbenzenesulfonate (30.0 mg, 1.0 equiv) and NaOH (29.1 mg, 20 equiv) in t-BuOH (1 mL) was stirred at 40° C. for 2 hours. The mixture was filtered and purified by prep-HPLC (column: Phenomenex luna C18 150×25 mm x m; mobile phase: [water (FA)-ACN]; B %: 18%-48%, 10 min) to afford the title compound (4.4 mg) as white solid (FA salt). ¹H NMR (400 MHz, METHANOL-d₄) δ=7.73-7.62 (m, 1H), 7.30-7.19 (m, 1H), 7.04-6.93 (m, 2H), 5.47-5.27 (m, 1H), 4.34-4.24 (m, 2H), 4.23-4.16 (m, 1H), 4.04-3.93 (m, 1H), 3.91-3.81 (m, 1H), 3.69-3.57 (m, 2H), 3.55-3.43 (m, 4H), 3.42-3.37 (m, 2H), 3.21-3.10 (m, 4H), 2.69-2.58 (m, 1H), 2.46-2.27 (m, 2H), 2.24-2.17 (m, 1H), 2.15-2.04 (m, 3H), 2.03-1.82 (m, 4H); LCMS (ESI, M+1): m/z=669.0, 671.0.

### Example 483

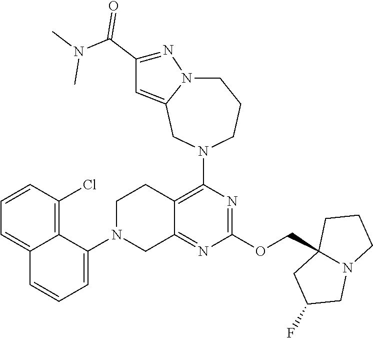 [SMILES: CN(C)C(=O)c1cc2n(n1)CCCN(c1nc(OC[C@@]34CCCN3C[C@H](F)C4)nc3c1CCN(c1cccc4cccc(Cl)c14)C3)C2]

### 5-(7-(8-chloronaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide

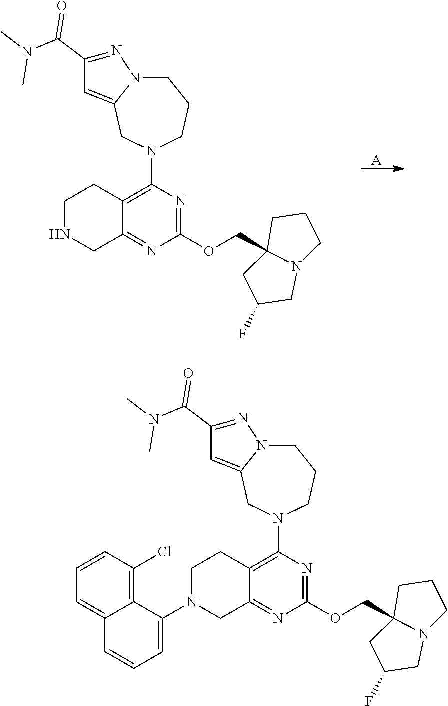

Step A. 5-(7-(8-chloronaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide: A mixture of 5-(2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide (70.0 mg, 1.0 equiv), 1-bromo-8-chloro-naphthalene (44.1 mg, 1.3 equiv), Pd₂(dba)₃ (12.9 mg, 0.1 equiv), RuPhos (13.1 mg, 0.2 equiv), and Cs₂CO₃ (137 mg, 3.0 equiv) in toluene (2 mL) were degassed and purged with nitrogen 3 times, and then the mixture was stirred at 90° C. for 4 hours under N₂ atmosphere. The reaction was diluted with water (20 mL) and extracted with EtOAc (40 mL). The combined organic layers were washed with brine (30 mL), dried over anhydrous sodium sulfate, concentrated, and purified with prep-HPLC [column: Phenomenex Synergi Max-RP 250×50 mm×10 μm; mobile phase: [water (FA)-ACN]; B %: 20%-45%, 21 minutes] to afford the title compound (11.6 mg, 12% yield) as white solid (FA salt). ¹H NMR (400 MHz, METHANOL-d₄) δ=8.71-8.43 (m, 1H), 7.82 (d, J=8.0 Hz, 1H), 7.67 (d, J=8.0 Hz, 1H), 7.53 (br d, J=7.2 Hz, 1H), 7.48 (t, J=8.0 Hz, 1H), 7.40-7.33 (m, 1H), 7.29 (d, J=7.2 Hz, 1H), 6.58 (s, 1H), 5.49-5.20 (m, 1H), 5.04-4.91 (m, 3H), 4.81 (br d, J=3.2 Hz, 2H), 4.59-4.43 (m, 2H), 4.26 (br d, J=17.2 Hz, 1H), 4.22-4.09 (m, 3H), 4.05 (br d, J=4.0 Hz, 1H), 3.69 (br d, J=17.4 Hz, 1H), 3.64-3.54 (m, 1H), 3.48-3.33 (m, 3H), 3.26 (br d, J=5.6 Hz, 1H), 3.20-3.10 (m, 2H), 3.08 (s, 3H), 2.67 (br d, J=15.2 Hz, 1H), 2.42-2.12 (m, 4H), 2.11-1.83 (m, 4H); LCMS (ESI, M+1): m/z=659.4

### Example 484

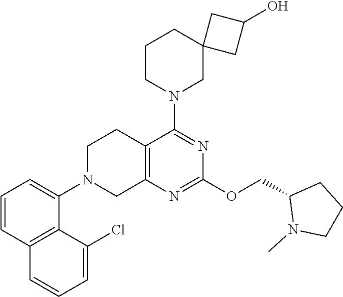 [SMILES: CN1CCC[C@H]1COc1nc2c(c(N3CCCC4(CC(O)C4)C3)n1)CCN(c1cccc3cccc(Cl)c13)C2]

### (S)-6-(7-(8-chloronaphthalen-1-yl)-2-((1-methylpyrrolidin-2-yl)methoxy)-5,6,7,8-tetrahydropyrido[3,4-d]pyrimidin-4-yl)-6-azaspiro[3.5]nonan-2-ol

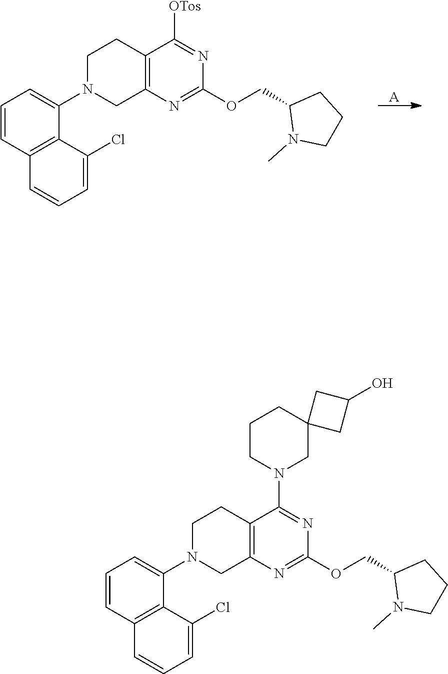

Step A. (S)-6-(7-(8-chloronaphthalen-1-yl)-2-((1-methylpyrrolidin-2-yl)methoxy)-5,6,7,8-tetrahydropyrido[3,4-d]pyrimidin-4-yl)-6-azaspiro[3.5]nonan-2-ol: To a solution of (S)-7-(8-chloronaphthalen-1-yl)-2-((1-methylpyrrolidin-2-yl)methoxy)-5,6,7,8-tetrahydropyrido[3,4-d]pyrimidin-4-yl 4-methylbenzenesulfonate (50.0 mg, 1.0 equiv) and 6-azaspiro[3.5]nonan-2-ol (14.6 mg, 1.2 equiv) in DMF (1.5 mL) and MeCN (1.5 mL) were added K₃PO₄ (55.0 mg, 3.0 equiv) and 4 Å molecular sieve (5.0 mg). The mixture was stirred at 60° C. for 12 hours. The reaction was concentrated and purified with prep-HPLC [column: Phenomenex Synergi C18 150 x 25 mm×10 μm; mobile phase: [water (FA)-ACN]; B %: 15%-45%, 10 minutes] and [column: DAICEL CHIRALPAK AD (250 mm×30 mm×10 μm); mobile phase: [0.1% NH₃•H₂O IPA]; B %: 45%-45%, 6 minutes] to afford the title compound (3.60 mg, 7.6% yield) as yellow solid. ¹H NMR (400 MHz, METHANOL-d₄) δ=7.72 (dd, J=0.8, 8.2 Hz, 1H), 7.57 (d, J=7.6 Hz, 1H), 7.43 (dd, J=1.2, 7.4 Hz, 1H), 7.39 (t, J=7.6 Hz, 1H), 7.27 (t, J=7.6 Hz, 1H), 7.21 (d, J=7.6 Hz, 1H), 4.31-4.24 (m, 1H), 4.24-4.11 (m, 3H), 3.71-3.63 (m, 1H), 3.58 (d, J=17.6 Hz, 1H), 3.53-3.43 (m, 2H), 3.34 (d, J=12.8 Hz, 1H), 3.18-2.98 (m, 4H), 2.76-2.65 (m, 1H), 2.50 (br d, J=14.0 Hz, 1H), 2.43 (s, 3H), 2.34-2.20 (m, 2H), 2.09-1.95 (m, 2H), 1.78-1.69 (m, 2H), 1.68-1.50 (m, 7H); LCMS (ESI, M+1): m/z=548.3.

### Example 485

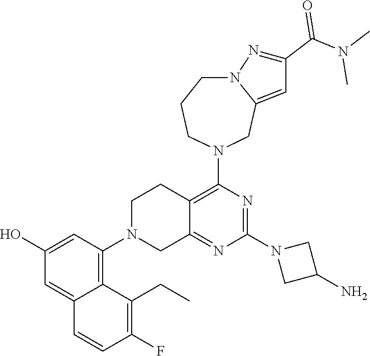 [SMILES: CCc1c(F)ccc2cc(O)cc(N3CCc4c(nc(N5CC(N)C5)nc4N4CCCn5nc(C(=O)N(C)C)cc5C4)C3)c12]

### 5-(2-(3-aminoazetidin-1-yl)-7-(8-ethyl-7-fluoro-3-hydroxynaphthalen-1-yl)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide

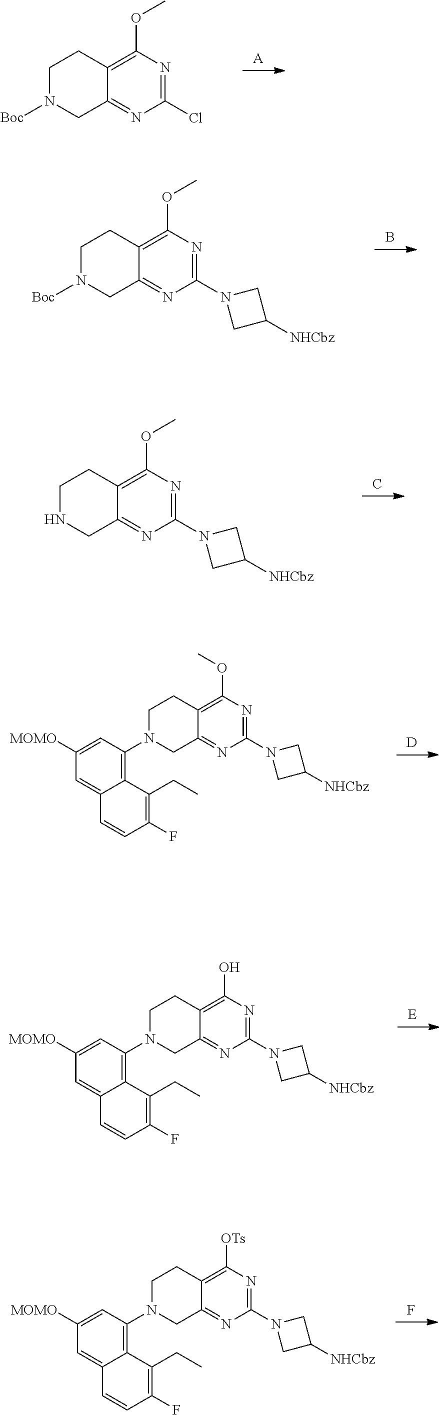

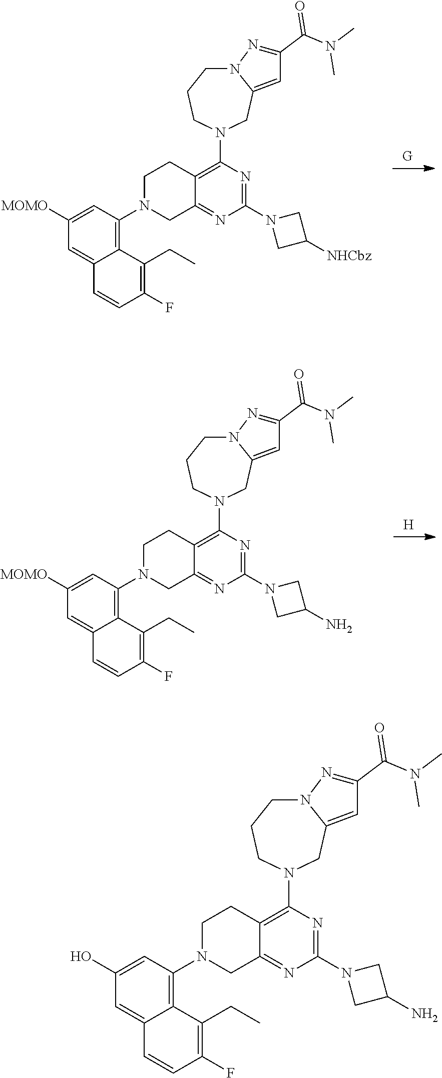

Step A. tert-butyl 2-(3-(((benzyloxy)carbonyl)amino)azetidin-1-yl)-4-methoxy-5,6-dihydropyrido[3,4-d]pyrimidine-7(8H)-carboxylate: To a mixture of tert-butyl 2-chloro-4-methoxy-5,6-dihydropyrido[3,4-d]pyrimidine-7(8H)-carboxylate (2.91 g, 1.0 equiv), benzyl azetidin-3-ylcarbamate (3.00 g, 1.5 equiv), (R)-(+)-2,2′-bis(diphenylphosphino)-1,1′-binaphthyl (845 mg, 0.20 equiv) and cesium carbonate (6.64 g, 3.0 equiv) in toluene (30 mL) was added palladium acetate (152 mg, 0.10 equiv). The reaction was degassed and purged with nitrogen 3 times. The reaction was stirred at 100° C. for 2 hours. The mixture was diluted with dichloromethane (40 mL) and methanol (10 mL), filtered, concentrated, and purified with column chromatography (silicon dioxide, Petroleum ether/Ethyl acetate=10/1 to 1/1). The product was further purified by reversed phase chromatography HPLC (0.1% FA condition) to afford the title compound (1.50 g, 45% yield) as yellow solid. LCMS (ESI, M+1): m/z=470.2.

Step B. benzyl (1-(4-methoxy-5,6,7,8-tetrahydropyrido[3,4-d]pyrimidin-2-yl)azetidin-3-yl)carbamate: A mixture of tert-butyl 2-(3-(((benzyloxy)carbonyl)amino)azetidin-1-yl)-4-methoxy-5,6-dihydropyrido[3,4-d]pyrimidine-7(8H)-carboxylate (1.40 g, 1.0 equiv) in HCl•MeOH (4 M, 10 mL, 13 equiv) was stirred at 20° C. for 1 hour. The mixture was concentrated under vacuum and the pH was adjusted to 11 with NaOH solution. The mixture was extracted with EtOAc (3×50 mL). The combined organic phase was washed with brine (50 mL), dried over anhydrous sodium sulfate, and concentrated to afford the title compound (0.910 g, 79% yield) as white solid. LCMS (ESI, M+1):m/z=370.1.

Step C. benzyl (1-(7-(8-ethyl-7-fluoro-3-(methoxymethoxy)naphthalen-1-yl)-⁴-methoxy-5,6,7,8-tetrahydropyrido[3,4-d] pyrimidin-2-yl)azetidin-3-yl)carbamate: To a mixture of benzyl (1-(4-methoxy-5,6,7,8-tetrahydropyrido[3,4-d]pyrimidin-2-yl)azetidin-3-yl)carbamate (0.91 g, 1.0 equiv), 8-ethyl-7-fluoro-3-(methoxymethoxy)naphthalen-1-yl trifluoromethanesulfonate (1.88 g, 2.0 equiv), Xantphos (285 mg, 0.20 equiv) and cesium carbonate (2.41 g, 3.0 equiv) in toluene was added tris(dibenzylideneacetone)dipalladium(0) (226 mg, 0.10 equiv). The reaction was degassed and purged with nitrogen 3 times. The reaction was stirred at 100° C. for 16 hours. The mixture was diluted with dichloromethane (20 mL) and methanol (10 mL), filtered, concentrated, and purified with column chromatography (silicon dioxide, Petroleum ether/Ethyl acetate=10/1 to 3/1) to afford the title compound (0.500 g, 32% yield) as white solid. LCMS (ESI, M+1): m/z=602.3.

Step D. benzyl (1-(7-(8-ethyl-7-fluoro-3-(methoxymethoxy)naphthalen-1-yl)-4-hydroxy-5,6,7,8-tetrahydropyrido[3,4-d]pyrimidin-2-yl)azetidin-3-yl)carbamate: To a solution of ethanethiol (310 mg, 6.0 equiv) in dimethylacetamide (2 mL) was added NaH (100 mg, 3.0 equiv, 60% purity) at 0° C. The reaction was stirred at 0° C. for 0.5 hour, and then a solution of benzyl (1-(7-(8-ethyl-7-fluoro-3-(methoxymethoxy)naphthalen-1-yl)-4-methoxy-5,6,7,8-tetrahydropyrido[3,4-d]pyrimidin-2-yl)azetidin-3-yl)carbamate (0.500 g, 1.0 equiv) in dimethylacetamide (3 mL) was added. The reaction was stirred at 60° C. for 0.5 hour. The mixture was diluted with water (10 mL) and extracted with EtOAc (2×20 mL). The combined organic phase was washed with brine (30 mL), dried over anhydrous sodium sulfate, and concentrated to afford the title compound (0.500 g, 32% yield) as black oil. LCMS (ESI, M+1):m/z=588.6.

Step E. 2-(3-(((benzyloxy)carbonyl)amino)azetidin-1-yl)-7-(8-ethyl-7-fluoro-3-(methoxymethoxy)naphthalen-1-yl)-5,6,7,8-tetrahydropyrido[3,4-d]pyrimidin-4-yl 4-methylbenzenesulfonate: To a solution of benzyl (1-(7-(8-ethyl-7-fluoro-3-(methoxymethoxy)naphthalen-1-yl)-4-hydroxy-5,6,7,8-tetrahydropyrido[3,4-d]pyrimidin-2-yl)azetidin-3-yl)carbamate (200 mg, 1.0 equiv) and 4-methylbenzenesulfonyl chloride (71.4 mg, 1.1 equiv) in dichloromethane (2.0 mL) were added diisopropylethylamine (132 mg, 3.0 equiv) and DMAP (4.16 mg, 0.10 equiv). The reaction was stirred at 25° C. for 1 hour. The mixture was concentrated and purified with column chromatography (silicon dioxide, Petroleum ether/Ethyl acetate=5/1 to 1/1) to afford the title compound (170 mg, 64% yield) as orange solid. LCMS (ESI, M+1): m/z=742.7.

Step F. benzyl (1-(5-(2-(dimethylcarbamoyl)-7,8-dihydro-4H-pyrazolo[1,5-a][1,4]diazepin-5(6H)-yl)-7-(8-ethyl-7-fluoro-3-(methoxymethoxy)naphthalen-1-yl)-5,6,7,8-tetrahydropyrido[3,4-d]pyrimidin-2-yl)azetidin-3-yl)carbamate: To a mixture 2-(3-(((benzyloxy)carbonyl)amino)azetidin-1-yl)-7-(8-ethyl-7-fluoro-3-(methoxymethoxy)naphthalen-1-yl)-5,6,7,8-tetrahydropyrido[3,4-d]pyrimidin-4-yl 4-methylbenzenesulfonate (100 mg, 1.0 equiv), N,N-dimethyl-5,6,7,8-tetrahydro-4H-pyrazolo[1,5-a][1,4]diazepine-2-carboxamide (140 mg, 5.0 equiv) and 4 Å molecular sieve (20.0 mg) in DMF (1 mL) was added DIEA (87.0 mg, 5.0 equiv). The reaction was stirred at 40° C. for 12 hours. The mixture was filtered and purified by reversed phase chromatography chromatography [C18, 0.1% formic acid condition] to afford the title compound (95.0 mg, 89% yield) as white solid. LCMS (ESI, M+1): m/z=778.6.

Step G. 5-(2-(3-aminoazetidin-1-yl)-7-(8-ethyl-7-fluoro-3-(methoxymethoxy)naphthalen-1-yl)-⁵,6,7,8-tetrahydropyrido[3,4-d] pyrimidin-5-yl)-N,N-dimethyl-5,6,7,8-tetrahydro-4H-pyrazolo[1,5-a][1,4]diazepine-2-carboxamide: To a mixture of benzyl (1-(5-(2-(dimethylcarbamoyl)-7,8-dihydro-4H-pyrazolo[1,5-a][1,4]diazepin-5(6H)-yl)-7-(8-ethyl-7-fluoro-3-(methoxymethoxy)naphthalen-1-yl)-5,6,7,8-tetrahydropyrido[3,4-d]pyrimidin-2-yl)azetidin-3-yl)carbamate (90.0 mg, 1.0 equiv) in MeOH (5 mL) was added Pd/C (10 mg, 10% purity) under N₂ atmosphere. The reaction was stirred at 20° C. for 10 min under H₂ at 15 psi. The mixture was filtered and concentrated under vacuum to afford the title compound (80.0 mg, 92% yield) as white solid. LCMS (ESI, M+1): m/z=644.7

Step H. 5-(2-(3-aminoazetidin-1-yl)-7-(8-ethyl-7-fluoro-3-hydroxynaphthalen-1-yl)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide: A mixture of 5-(2-(3-aminoazetidin-1-yl)-7-(8-ethyl-7-fluoro-3-(methoxymethoxy)naphthalen-1-yl)-5,6,7,8-tetrahydropyrido[3,4-d]pyrimidin-5-yl)-N,N-dimethyl-5,6,7,8-tetrahydro-4H-pyrazolo[1,5-a][1,4]diazepine-2-carboxamide (50.0 mg, 1 equiv) and HCl•MeOH (4 M, 1.5 mL, 77.0 equiv) was stirred at 20° C. for 0.5 hour. The reaction was concentrated under vacuum with oil pump, neutralized to pH=8 with NaHCO₃ solution (2 ml) and extracted with ethyl acetate (2×3 mL). The combined organic layers were washed with brine (3 mL), dried over anhydrous sodium sulfate, concentrated, and purified with prep-HPLC [column: Phenomenex luna C18 150×25 mm×10 μm; mobile phase: [water (FA)-ACN]; B %: 6%-36%, 10 min] to afford the title compound (6.60 mg, 13% yield, HCOOH salt) as yellow solid. ¹H NMR (400 MHz, METHANOL-d₄) δ=7.6 (dd, J=5.6, 8.8 Hz, 1H), 7.25-7.10 (m, 1H), 7.03-6.90 (m, 2H), 6.62-6.52 (m, 1H), 4.95 (br s, 1H), 4.82-4.76 (m, 1H), 4.61-4.51 (m, 2H), 4.36-4.24 (m, 2H), 4.17-3.87 (m, 6H), 3.66-3.48 (m, 3H), 3.40 (br d, J=7.6 Hz, 4H), 3.25-3.07 (m, 5H), 2.74-2.63 (m, 1H), 2.37-2.23 (m, 1H), 2.09-1.95 (m, 1H), 1.19-1.05 (m, 3H); LCMS (ESI, M+1): m/z=600.4.

### Example 486

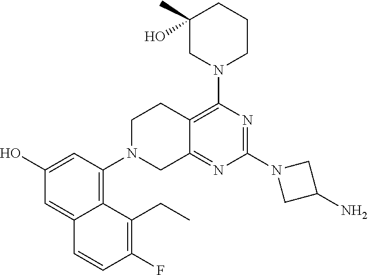 [SMILES: CCc1c(F)ccc2cc(O)cc(N3CCc4c(nc(N5CC(N)C5)nc4N4CCC[C@@](C)(O)C4)C3)c12]

### (R)-1-(2-(3-aminoazetidin-1-yl)-7-(8-ethyl-7-fluoro-3-hydroxynaphthalen-1-yl)-5,6,7,8-tetrahydropyrido[3,4-d]pyrimidin-4-yl)-3-methylpiperidin-3-ol

Synthesized according to Example 485. The title compound was obtained as yellow solid (TFA salt). ¹H NMR (400 MHz, METHANOL-d₄) δ=7.53 (dd, J=6.0, 9.2 Hz, 1H), 7.17 (t, J=9.2 Hz, 1H), 7.07-6.97 (m, 2H), 4.62-4.51 (m, 2H), 4.47-4.08 (m, 5H), 4.07-3.88 (m, 1H), 3.83-3.53 (m, 2H), 3.52-3.44 (m, 1H), 3.43-3.34 (m, 2H), 3.30-3.03 (m, 3H), 2.85 (br d, J=15.2 Hz, 0.5H), 2.66 (br d, J=13.6 Hz, 0.5H), 2.13-1.86 (m, 1H), 1.84-1.62 (m, 3H), 1.24 (d, J=15.6 Hz, 3H), 1.14 (dt, J=4.8, 7.2 Hz, 3H); LCMS (ESI, M+1): m/z=507.3.

### Example 487

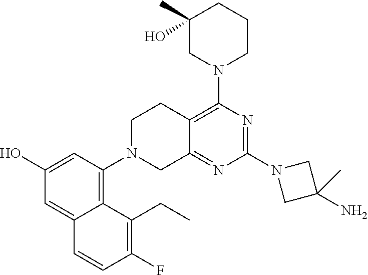 [SMILES: CCc1c(F)ccc2cc(O)cc(N3CCc4c(nc(N5CC(C)(N)C5)nc4N4CCC[C@@](C)(O)C4)C3)c12]

### (R)-1-(2-(3-amino-3-methylazetidin-1-yl)-7-(8-ethyl-7-fluoro-3-hydroxynaphthalen-1-yl)-5,6,7,8-tetrahydropyrido[3,4-d]pyrimidin-4-yl)-3-methylpiperidin-3-ol

Synthezied according to Example 427. The title compound was obtained as orange solid (FA salt). ¹H NMR (400 MHz, methanoL-d₄) δ=7.55-7.47 (m, 1H), 7.18-7.10 (m, 1H), 7.01-6.93 (m, 2H), 4.13-3.95 (m, 5H), 3.78-3.70 (m, 0.5H), 3.65-3.53 (m, 2H), 3.53-3.39 (m, 3H), 3.39-3.32 (m, 1.5H), 3.25-3.00 (m, 3H), 2.72-2.58 (m, 1H), 2.02-1.63 (m, 4H), 1.61 (s, 3H), 1.31-1.17 (m, 3H), 1.16-1.06 (m, 3H); ¹⁹F NMR (400 MHz, methanoL-d₄) δ=−123.187; LCMS (ESI, M+1): m/z=521.2.

### Example 488

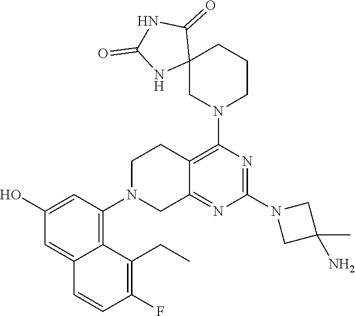 [SMILES: CCc1c(F)ccc2cc(O)cc(N3CCc4c(nc(N5CC(C)(N)C5)nc4N4CCCC5(C4)NC(=O)NC5=O)C3)c12]

### 7-(2-(3-amino-3-methylazetidin-1-yl)-7-(8-ethyl-7-fluoro-3-hydroxynaphthalen-1-yl)-5,6,7,8-tetrahydropyrido[3,4-d]pyrimidin-4-yl)-1,3,7-triazaspiro[4.5]decane-2,4-dione

Synthezied according to Example 427. The title compound was obtained as yellow solid (FA salt). ¹H NMR (400 MHz, methanol-d₄) δ=7.55-7.47 (m, 1H), 7.19-7.10 (m, 1H), 7.01-6.91 (m, 2H), 4.15-3.79 (m, 7H), 3.67-3.54 (m, 1H), 3.53-3.32 (m, 3.5H), 3.28-3.23 (m, 0.5H), 3.20-2.85 (m, 3H), 2.82-2.65 (m, 1H), 2.15-1.72 (m, 4H), 1.66-1.49 (m, 3H), 1.18-0.99 (m, 3H); ¹⁹F NMR (400 MHz, methanol-d₄) δ=−123.179; LCMS (ESI, M+1): m/z=575.2.

### Example 489

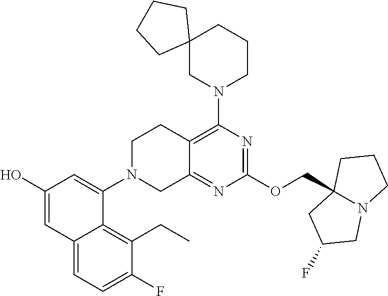 [SMILES: CCc1c(F)ccc2cc(O)cc(N3CCc4c(nc(OC[C@@]56CCCN5C[C@H](F)C6)nc4N4CCCC5(CCCC5)C4)C3)c12]

### 5-ethyl-6-fluoro-4-(2-(((2R,7aS)-2-fluorohexahydro-1H-pyrrolizin-7a-yl)methoxy)-4-(7-azaspiro[4.5]decan-7-yl)-5,6-dihydropyrido[3,4-d]pyrimidin-7(8H)-yl)naphthalen-2-ol

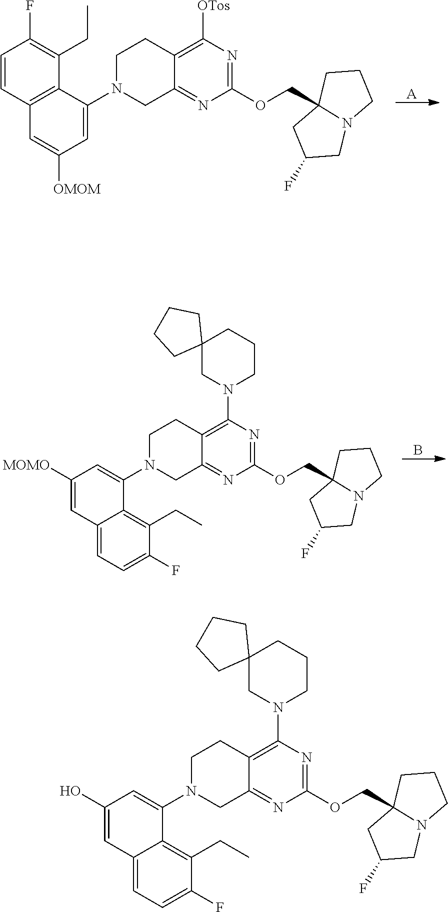

Synthesized according to Example 248. The title compound was obtained yellow oil (FA salt). ¹H NMR (400 MHz, METHANOL-d₄) δ=7.51 (dd, J=6.0, 9.2 Hz, 1H), 7.14 (t, J=9.6 Hz, 1H), 6.99-6.94 (m, 2H), 5.49-5.29 (m, 1H), 4.38-4.21 (m, 2H), 4.07 (br d, J=17.6 Hz, 1H), 4.02-3.94 (m, 1H), 3.71-3.58 (m, 2H), 3.57-3.47 (m, 3H), 3.44-3.34 (m, 2H), 3.28-3.11 (m, 5H), 2.71-2.58 (m, 1H), 2.54-2.28 (m, 2H), 2.26-2.06 (m, 3H), 2.05-1.93 (m, 1H), 1.89-1.77 (m, 1H), 1.76-1.27 (m, 12H), 1.10 (t, J=7.2 Hz, 3H); LCMS (ESI, M+1): m/z=618.4.

### Example 490

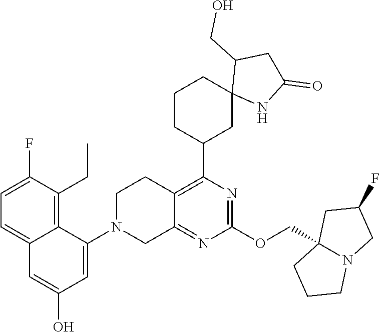 [SMILES: CCc1c(F)ccc2cc(O)cc(N3CCc4c(nc(OC[C@@]56CCCN5C[C@H](F)C6)nc4C4CCCC5(C4)NC(=O)CC5CO)C3)c12]

### 7-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-4-(hydroxymethyl)-1,7-diazaspiro[4.5]decan-2-one

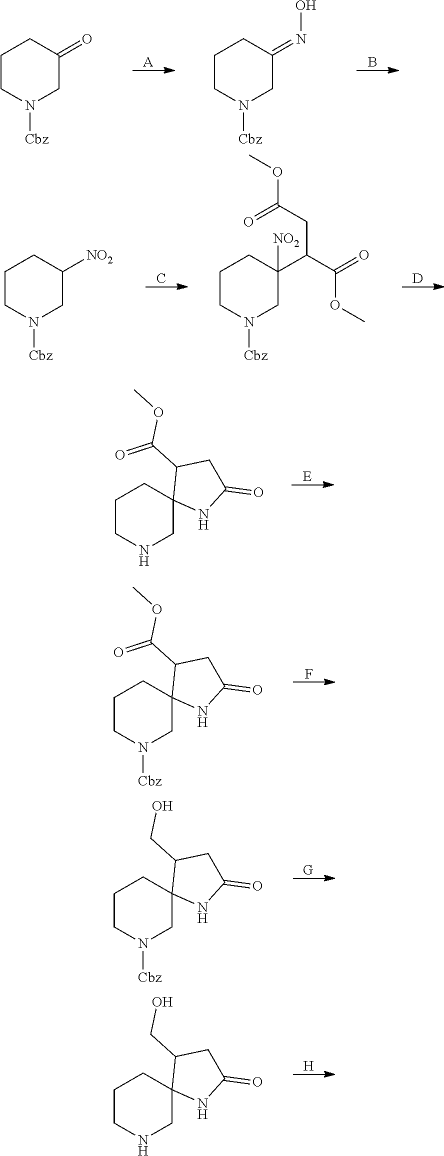

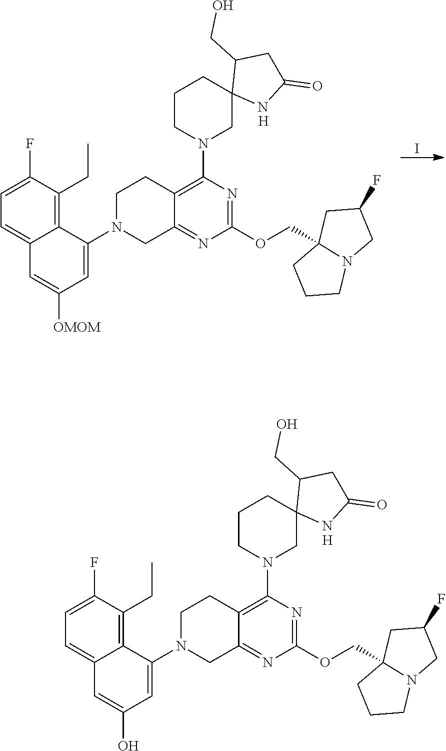

Step A. (E)-benzyl 3-(hydroxyimino)piperidine-1-carboxylate: To a mixture of benzyl 3-oxopiperidine-1-carboxylate (8.00 g, 1.0 equiv) and sodium acetate (11.2 g, 4.0 equiv) in methanol (80 mL) was added hydroxylamine hydrochloride (4.80 g, 2.0 equiv) under N₂ atmosphere. The reaction was stirred at 60° C. for 2 hours. The mixture was concentrated. The residue was diluted with water (40 mL) and extracted with ethyl acetate (3×30 mL). The combined organic phase was dried over Na₂SO₄ and concentrated to afford the title compound (10 g, crude) as yellow oil; LCMS (ESI, M+1): m/z=249.2.

Step B. benzyl 3-nitropiperidine-1-carboxylate: To a solution of urea hydrogen peroxide (12.0 g, 3.2 equiv) in acetonitrile (50 mL) was added (2,2,2-trifluoroacetyl) 2,2,2-trifluoroacetate (21.0 g, 2.5 equiv) dropwise at 0° C. The mixture was stirred at 0° C. for 2 hours and then added dropwise into a mixture of benzyl (3E)-3-hydroxyiminopiperidine-1-carboxylate (10.0 g, 1.0 equiv) and NaHCO₃ (17.0 g, 5.0 equiv) in acetonitrile (80 mL) at 70° C. The reaction was stirred at 80° C. for 2 hours. The mixture was quenched with saturated sodium sulfite aqueous (100 mL) below 15° C. The mixture was concentrated, diluted with water (200 mL) and extracted with dichloromethane (3×80 mL). The organic phase was dried over Na₂SO₄, concentrated and purified with reversed phase chromatography [0.1% formic acid condition] to afford the title compound (3.60 g, 35% yield) as yellow oil; LCMS (ESI, M+1): m/z=265.1.

Step C. dimethyl 2-(1-((benzyloxy)carbonyll)-3-nitropiperidin-3-yl)succinate: To a solution of benzyl 3-nitropiperidine-1-carboxylate (3.60 g, 1.0 equiv) in dioxane (20 mL) were added dimethyl (E)-but-2-enedioate (11.8 g, 6.0 equiv) and KF (4.70 g, 6.0 equiv). The reaction was stirred at 90° C. for 36 hours. The mixture was diluted with water (80 mL) and extracted with ethyl acetate (3×30 mL). The combined organic phase was dried over Na₂SO₄, concentrated and purified with reversed phase chromatography [0.1% formic acid condition] to afford the title compound (4.15 g, 74% yield) as yellow oil; LCMS (ESI, M+1): m/z=409.1.

Step D. methyl 2-oxo-1,7-diazaspiro[4.5]decane-4-carboxylate: To a solution of dimethyl 2-(1-benzyloxycarbonyl-3-nitro-3-piperidyl)butanedioate (1.00 g, 1.0 equiv) in ethanol (10 mL) was added Raney-Ni (100 mg). The mixture was degassed and purged with N₂ and then H₂ 3 times. The reaction was stirred at 60° C. for 5 hours under H₂ atmosphere (15 psi). The mixture was filtered and concentrated to afford the title compound (0.55 g, crude) as white solid; LCMS (ESI, M+1): m/z=213.1.

Step E. 7-benzyl 4-methyl 2-oxo-1,7-diazaspiro[4.5]decane-4,7-dicarboxylate: To a mixture of methyl 2-oxo-1,9-diazaspiro[4.5]decane-4-carboxylate (550 mg, 1.0 equiv) in H₂O (5.0 mL) and tetrahydrofuran (5.0 mL) was added NaHCO₃ (435 mg, 2.0 equiv). The reaction was stirred at 20° C. for 0.1 hour and benzyl chloroformate (464 mg, 1.0 equiv) was added dropwise. The reaction was stirred at 20° C. for 12 hours. The mixture was diluted with water (50 mL) and extracted with ethyl acetate (3×30 mL). The combined organic phase was dried over Na₂SO₄, concentrated and purified with reversed phase chromatography [0.1% formic acid condition] to afford the title compound (300 mg, 33% yield) as yellow oil; LCMS (ESI, M+1): m/z=347.1.

Step F. benzyl 4-(hydroxymethyl)-2-oxo-1,7-diazaspiro[4.5]decane-7-carboxylate: To a solution of 7-benzyl 4-methyl 2-oxo-1,7-diazaspiro[4.5]decane-4,7-dicarboxylate (0.30 g, 1.0 equiv) in tetrahydrofuran (3.0 mL) was added LiBH₄ (240 mg, 13 equiv) slowly at 0° C. under N₂ atmosphere. The reaction was stirred at 20° C. for 4 hours. The mixture was quenched with saturated NH₄Cl aqueous (20 mL) at 0° C., diluted with water (10 mL) and extracted with ethyl acetate (3×10 mL). The combined organic phase was dried over Na₂SO₄ and concentrated to afford the title compound (230 mg, crude) as yellow oil; LCMS (ESI, M+1): m/z=319.1.

Step G. 4-(hydroxymethyl)-1,7-diazaspiro[4.5]decan-2-one: To a solution of benzyl 4-(hydroxymethyl)-2-oxo-1,9-diazaspiro[4.5]decane-9-carboxylate (230 mg, 1.0 equiv) in methanol (8.0 mL) was added Pd/C (50 mg, 10% purity). The mixture was degassed and purged with N₂ and then H₂ for 3 times. The reaction was stirred at 20° C. for 1 hour under H₂ atmosphere (15 psi). The mixture was filtered and concentrated to afford the title compound (130 mg, crude) as white solid; 1H NMR (400 MHz, DMSO-d₆) δ=7.92-7.67 (m, 1H), 5.00-4.69 (m, 1H), 3.50 (br dd, J=6.8, 10.4 Hz, 1H), 3.32-3.15 (m, 2H), 2.79-2.57 (m, 2H), 2.46-2.23 (m, 2H), 2.19-2.13 (m, 1H), 2.12-1.87 (m, 2H), 1.70-1.38 (m, 4H).

The last two steps were performed according to Example 248. The title compound was obtained as white solid (FA salt). ¹H NMR (400 MHz, METHANOL-d₄) δ=8.50 (s, 1H), 7.51 (dd, J=6.0, 8.8 Hz, 1H), 7.15 (t, J=9.2 Hz, 1H), 6.98 (br d, J=4.4 Hz, 2H), 5.52-5.31 (m, 1H), 4.42-4.20 (m, 2H), 4.16-3.83 (m, 3H), 3.83-3.46 (m, 7H), 3.46-3.32 (m, 3H), 3.29-2.94 (m, 4H), 2.85-2.68 (m, 1H), 2.60-2.28 (m, 5H), 2.27-1.74 (m, 8H), 1.19-0.99 (m, 3H). LCMS (ESI, M+1): m/z=663.3.

### Example 491

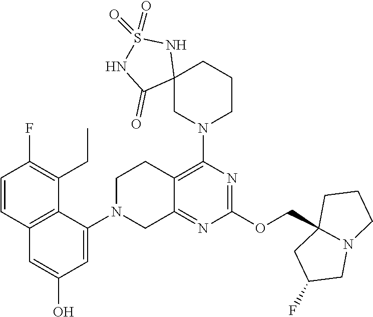 [SMILES: CCc1c(F)ccc2cc(O)cc(N3CCc4c(nc(OC[C@@]56CCCN5C[C@H](F)C6)nc4N4CCCC5(C4)NS(=O)(=O)NC5=O)C3)c12]

### 7-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-2-thia-1,3,7-triazaspiro[4.5]decan-4-one 2,2-dioxide

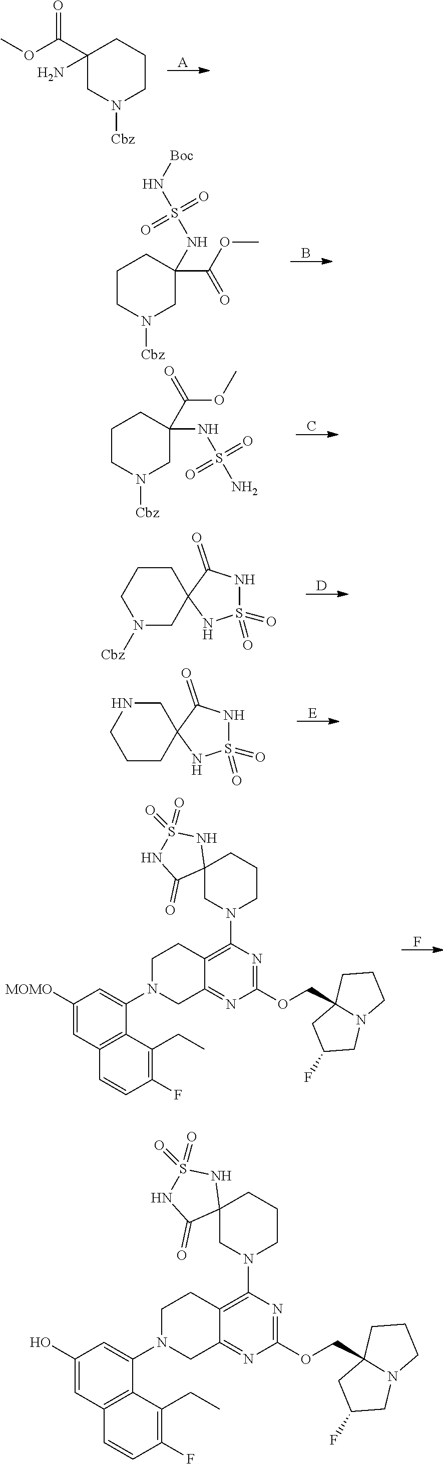

Step A. 1-benzyl 3-methyl 3-((N-(tert-PGP-⁵⁵²,C³ butoxycarbonyl)sulfamoyl)amino)piperidine-1,3-dicarboxylate: To a solution chlorosulfonyl isocyanate (96.8 mg, 1.0 equiv) in dichloromethhane (0.5 mL) was added a solution of tert-butyl alcohol (50.7 mg, 1.0 equiv) in dichloromethhane (0.5 mL) at 0° C. The mixture was stirred at 0° C. for 30 minutes. A mixture of 1-benzyl 3-methyl 3-aminopiperidine-1,3-dicarboxylate (200 mg, 1.0 equiv) and triethylamine (69.2 mg, 1.0 equiv) in dichloromethhane (2.5 mL) was added at 0° C. for 5 minutes. The reaction was stirred at 20° C. for 10 hours. The mixture was diluted with water (20 mL) and extracted with dichloromethhane (2×5 mL). The combined organic layers were washed with brine (20 mL), dried over Na₂SO₄, concentrated and purified with reversed phase chromatography [C18, 0.1% formic acid condition] to afford the title compound (50.0 mg, 6.6% yield) as white solid; LCMS (ESI, M-100+1): m/z=372.0.

Step B. 1-benzyl 3-methyl 3-(sulfamovlamino)piperidine-1,3-dicarboxylate: To a solution of 1-benzyl 3-methyl 3-((N-(tert-butoxycarbonyl)sulfamoyl)amino)piperidine-1,3-dicarboxylate (300 mg, 1.0 equiv) in methanol (2 mL) was added HCl•MeOH (4 M, 3.0 mL, 19 equiv) at 0° C. The reaction was stirred at 0° C. for 0.5 hours and then at 30° C. for 0.5 hours. The mixture was concentrated to afford the title compound (300 mg, crude) as yellow solid.

Step C. benzyl 4-oxo-2-thia-1,3,7-triazaspiro[4.5]decane-7-carboxylate 2,2-dioxide: To a solution of sodium methylate (174 mg, 4.0 equiv) in methanol (2 mL) was added 1-benzyl 3-methyl 3-(sulfamoylamino)piperidine-1,3-dicarboxylate (300 mg, 1.0 equiv) in methanol (1.5 mL) slowly. The mixture was stirred at 68° C. for 18 hours. The mixture was concentrated, diluted with water (10 mL) and extracted with ethyl acetate (2×10 mL). The combined organic layers were washed with brine (10 mL), dried over Na₂SO₄ and concentrated to afford the title compound (300 mg, crude) as white solid.

Step D. 2-thia-1,3,7-triazaspiro[4.5]decan-4-one 2,2-dioxide: To a mixture of Pd/C (50.0 mg, 10% purity) and methanol (2 mL) was added benzyl 4-oxo-2-thia-1,3,7-triazaspiro[4.5]decane-7-carboxylate 2,2-dioxide (150 mg, 1.0 equiv) under N₂ atmosphere. The mixture was degassed and purged with N₂ and then H₂ for 3 times. The mixture was stirred at 30° C. for 1 hour under H₂ atmosphere (15 psi). The mixture was filtered and concentrated to afford the title compound (80.0 mg, crude) as white solid.

The last two steps were performed according to Example 248. The title compound was obtained as white solid. 1H NMR (400 MHz, METHANOL-d₄) δ=7.56-7.47 (m, 1H), 7.15 (dt, J=3.2, 9.2 Hz, 1H), 7.03-6.90 (m, 2H), 5.65-5.43 (m, 1H), 4.64-4.54 (m, 2H), 4.54-4.37 (m, 2H), 4.16-4.05 (m, 2H), 4.04-3.83 (m, 2H), 3.82-3.67 (m, 2H), 3.64-3.46 (m, 2H), 3.45-3.34 (m, 4H), 3.21-3.06 (m, 2H), 2.77-2.56 (m, 2H), 2.56-2.35 (m, 2H), 2.33-2.09 (m, 4H), 1.93-1.80 (m, 2H), 1.15-1.03 (m, 3H); LCMS (ESI, M+1): m/z=684.3.

### Example 492

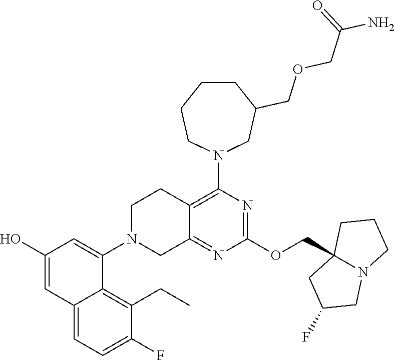 [SMILES: CCc1c(F)ccc2cc(O)cc(N3CCc4c(nc(OC[C@@]56CCCN5C[C@H](F)C6)nc4N4CCCCC(COCC(N)=O)C4)C3)c12]

### 2-((1-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)azepan-3-yl)methoxy)acetamide

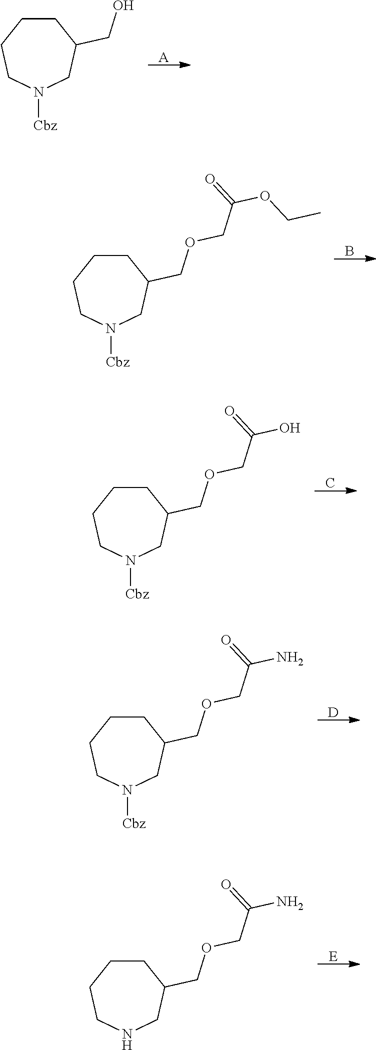

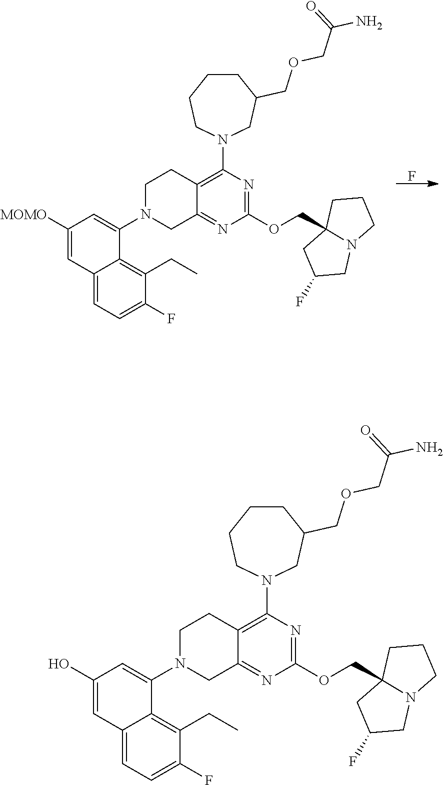

Step A. benzyl 3-((2-ethoxy-2-oxoethoxy) methyl)azepane-1-carboxylate: To a solution of benzyl 3-(hydroxymethyl)azepane-1-carboxylate (500 mg, 1.0 equiv) in THE (6.0 mL) were added NaH (228 mg, 60% purity, 3.0 equiv) and ethyl 2-bromoacetate (634 mg, 2.0 equiv) at 0° C. The reaction was stirred at 20° C. for 6 hours. The mixture was quenched with saturated NH₄Cl (10.0 mL at 0° C.) and extracted with DCM (3×10 mL). The combined organic layers were washed with brine (10 mL), dried over Na₂SO₄, filtered and purified by prep-TLC [SiO₂, petroleum ether: ethyl acetate=2:1] to afford the title compound (350 mg, 53% yield) as a white solid; LCMS (ESI, M+1): m/z=350.2.

Step B. 2-((1-((benzyloxy)carbonyl)azepan-3-yl)methoxy)acetic acid: To a solution of benzyl 3-((2-ethoxy-2-oxoethoxy) methyl) azepane-1-carboxylate (350 mg, 1.0 equiv) in MeOH (5.0 mL) and water (1.0 mL) was added NaOH (120 mg, 3.0 equiv). The reaction was stirred at 60° C. for 2 hours. The mixture was filtered and concentrated to afford the title compound (300 mg, crude) as a white solid; LCMS (ESI, M+1): m/z=322.4.

Step C. benzyl 3-((2-amino-2-oxoethoxy) methyl) azepane-1-carboxylate: To a solution of 2-((1-((benzyloxy)carbonyl)azepan-3-yl)methoxy)acetic acid (300 mg, 1.0 equiv), HATU (710 mg, 2.0 equiv) and DIEA (362 mg, 3.0 equiv) in THF (5.0 mL) was added NH₄Cl (100 mg, 2.0 equiv). The reaction was stirred at 20° C. for 2 hours. The mixture was diluted with water (20 mL), extracted with DCM (3×20 mL). The combined organic layers were washed with brine (20 mL), dried over Na₂SO₄, filtered, and purified with prep-TLC [SiO₂, petroleum ether: ethyl acetate=0:1] to afford the title compound (200 mg, 67% yield) as a white solid; LCMS (ESI, M+1): m/z=321.4.

Step D. 2-(azepan-3-ylmethoxy) acetamide: To a solution of benzyl 3-((2-amino-2-oxoethoxy)methyl)azepane-1-carboxylate (30.0 mg, 1.0 equiv) in EtOAc (2.0 mL) was added Pd/C (20.0 mg, 10% purity, 1.0 equiv). The reaction was stirred under H₂ (15 psi) at 20° C. for 2 hours. The mixture was filtered and concentrated to afford the title compound (20.0 mg, crude) as a white solid.

The last two steps were performed according to Example 248. The title compound was obtained as as a yellow solid; ¹H NMR (400 MHz, METHANOL-d₄) δ=7.51 (dd, J=6.0, 9.2 Hz, 1H), 7.14 (t, J=9.2 Hz, 1H), 7.01-6.93 (m, 2H), 5.37-5.17 (m, 1H), 4.35-3.95 (m, 1H), 3.94 (d, J=0.8 Hz, 2H), 3.75-3.34 (m, 7H), 3.29-3.09 (m, 6H), 3.05-2.91 (m, 1H), 2.82-2.70 (m, 1H), 2.52-2.24 (m, 1H), 2.21-1.66 (m, 10H), 1.52-1.35 (m, 2H), 1.17-1.02 (m, 3H);LCMS (ESI, M+1): m/z=665.6.

### Example 493

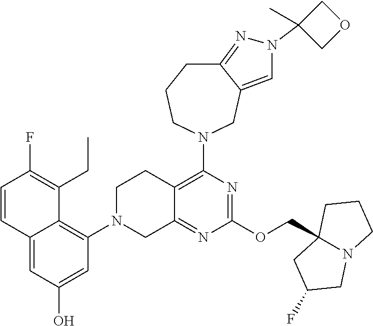 [SMILES: CCc1c(F)ccc2cc(O)cc(N3CCc4c(nc(OC[C@@]56CCCN5C[C@H](F)C6)nc4N4CCCc5nn(C6(C)COC6)cc5C4)C3)c12]

### 5-ethyl-6-fluoro-4-(2-(((2R,7aS)-2-fluorohexahydro-1H-pyrrolizin-7a-yl)methoxy)-4-(2-(3-methyloxetan-3-yl)-7,8-dihydropyrazolo[4,3-c]azepin-5(2H,4H,6H)-yl)-5, 6-dihydropyrido[3,4-d]pyrimidin-7(8H)-yl)naphthalen-2-ol

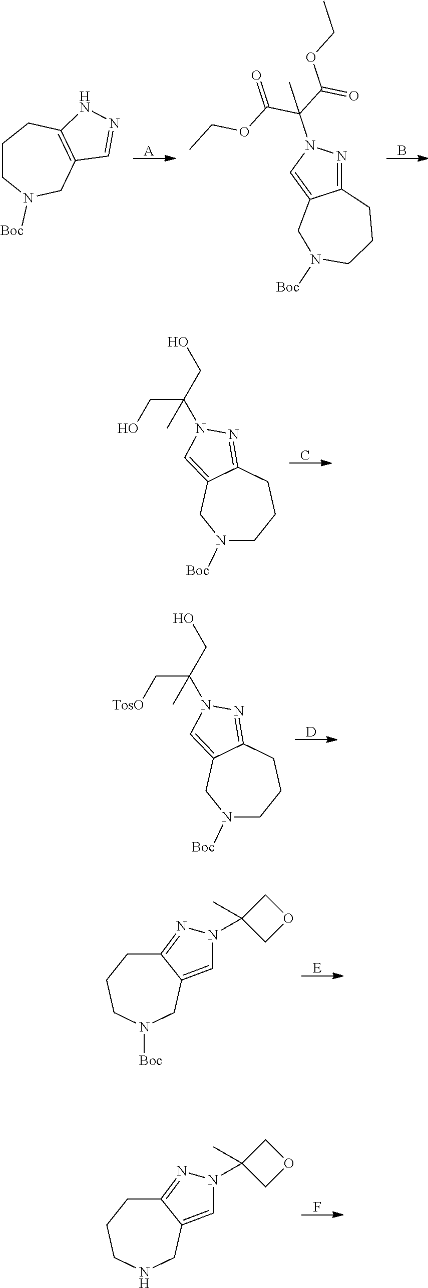

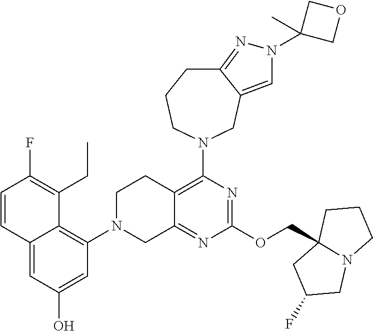 [SMILES: CCc1c(F)ccc2cc(O)cc(N3CCc4c(nc(OC[C@@]56CCCN5C[C@H](F)C6)nc4N4CCCc5nn(C6(C)COC6)cc5C4)C3)c12]

Step A. diethyl 2-(5-(tert-butoxycarbonyl)-5,6,7,8-tetrahydropyrazolo[4,3-c]azepin-2(4H)-yl)-2-methylmalonate: To a solution of tert-butyl 4,6,7,8-tetrahydropyrazolo[4,3-c]azepine-5(1H)-carboxylate (7.00 g, 1.0 equiv) in THF (100 mL) was added portion-wise NaH (1.77 g, 60% purity, 1.5 equiv) at 0° C. The reaction was stirred at 0° C. for 30 minutes. Then diethyl 2-bromo-2-methyl-propanedioate (8.96 g, 1.2 equiv) was added dropwise at 0° C. The reaction was stirred at 20° C. for 11.5 hours. The mixture was diluted with water (10 mL) and extracted with ethyl acetate (5×50 mL). The combined organic layers were washed with brine (90 mL), dried over anhydrous sodium sulfate, concentrated and purified with column chromatography [SiO₂, Petroleum ether/Ethyl acetate=I/O to 1/1] to afford the title compound (7.00 g, 58% yield) as colorless liquid; 1H NMR (400 MHz, CHLOROFORM-d) δ=7.44 (d, J=17.6 Hz, 1H), 4.36-4.19 (m, 6H), 3.62 (s, 2H), 2.90-2.78 (m, 2H), 2.05 (s, 3H), 1.78 (s, 2H), 1.38 (d, J=9.6 Hz, 9H), 1.25 (d, J=7.2 Hz, 6H); LCMS (ESI, M+1): m/z=410.3.

Step B. tert-butyl 2-(1,3-dihydroxy-2-methylpropan-2-yl)-4,6,7,8-tetrahydropyrazolo[4,3-c]azepine-5(2H)-carboxylate: To a solution of diethyl 2-(5-(tert-butoxycarbonyl)-5,6,7,8-tetrahydropyrazolo[4,3-c]azepin-2(4H)-yl)-2-methylmalonate (7.00 g, 1.0 equiv) in ethanol (70 mL) was added portion-wise NaBH4 (3.25 g, 5.0 equiv) at 0° C. The reaction was stirred at 20° C. for 12 hours. The mixture was concentrated, diluted with water (10 mL) and extracted with ethyl acetate (3×10 mL). The combined organic layers were dried over anhydrous sodium sulfate, concentrated, and purified with reversed phase chromatography chromatography [C18, 0.1% formic acid condition] to afford the title compound (3.00 g, 53% yield) as colorless liquid; LCMS (ESI, M+1): m/z=326.2.

Step C. tert-butyl 2-(1-hydroxy-2-methyl-3-(tosvloxy)propan-2-yl)-4,6,7,8-tetrahydropyrazolo[4,3-c]azepine-5(2H)-carboxylate: To a solution of tert-butyl 2-(1,3-dihydroxy-2-methylpropan-2-yl)-4,6,7,8-tetrahydropyrazolo[4,3-c]azepine-5(2H)-carboxylate (1.65 g, 1.0 equiv) in THE (160 mL) was added dropwise n-BuLi (2.5 M, 2.23 mL, 1.1 equiv) at 0° C. The reaction was stirred at 0° C. for 0.5 hours. A solution of TsCl (1.06 g, 1.1 equiv) in THE (10 mL) was added dropwise at 0° C. The reaction was stirred at 0° C. for 1 hour. The mixture was diluted with water (20 mL) and extracted with ethyl acetate (3×20 mL). The combined organic layers were dried over anhydrous sodium sulfate, concentrated and purified with reversed phase chromatography chromatography [C18, 0.1% formic acid condition] to afford the title compound (1.30 g, 53% yield) as colorless liquid; ¹H NMR (400 MHz, CHLOROFORM-d) δ=7.68 (br s, 2H), 7.36-7.27 (m, 3H), 4.55-4.39 (m, 1H), 4.29-4.19 (m, 3H), 3.96-3.81 (m, 1H), 3.80-3.45 (m, 3H), 2.87-2.65 (m, 2H), 2.45 (s, 3H), 1.86-1.70 (m, 2H), 1.49 (br d, J=7.6 Hz, 3H), 1.43 (br s, 9H); LCMS (ESI, M+1): m/z=480.2.

Step D. tert-butyl 2-(3-methyloxetan-3-yl)-4,6,7,8-tetrahydropyrazolo[4,3-c]azepine-5(2H)-carboxylate: To a solution of tert-butyl 2-(1-hydroxy-2-methyl-3-(tosyloxy)propan-2-yl)-4,6,7,8-tetrahydropyrazolo[4,3-c]azepine-5(2H)-carboxylate (700 mg, 1.0 equiv) in THE (7 mL) was added portion-wise NaH (70.0 mg, 60% purity, 1.2 equiv) at 0° C. The reaction was stirred at 65° C. for 2 hours. The reaction was diluted with water (20 mL) and extracted with ethyl acetate (3×20 mL). The combined organic layers were dried over anhydrous sodium sulfate, concentrated and purified with reversed phase chromatography chromatography [C18, 0.1% formic acid condition] to afford the title compound (340 mg, 75% yield) as colorless liquid; ¹H NMR (400 MHz, CHLOROFORM-d) δ=7.32 (br s, 1H), 5.13 (br d, J=5.6 Hz, 2H), 4.75-4.48 (m, 2H), 4.41-4.18 (m, 2H), 3.66 (br s, 2H), 3.01-2.81 (m, 2H), 1.89 (s, 3H), 1.82 (br s, 2H), 1.42 (br s, 9H); LCMS (ESI, M+1): m/z=308.2.

Step E. 2-(3-methyloxetan-3-yl)-²,4,5,6,7,8-hexahydropyrazolo[4,3-c]azepine: To a solution of tert-butyl 2-(3-methyloxetan-3-yl)-4,6,7,8-tetrahydropyrazolo[4,3-c]azepine-5-carboxylate (80.0 mg, 1.0 equiv) in dichloromethane (0.5 mL) was added TFA (770 mg, 26 equiv) at 0° C. The reaction was stirred at 0° C. for 0.5 hours. The reaction was concentrated to afford the title compound (80.0 mg, crude, TFA salt) as yellow liquid.

Step F. 5-ethyl-6-fluoro-4-(2-(((2R,7aS)-2-fluorohexahydro-1H-pyrrolizin-7a-yl)methoxy)-4-(2-(3-methyloxetan-3-yl)-⁷,8-dihydropyrazolo[4,3-c]azepin-5(2H,4H,6H)-yl)-5,6-dihydropyrido[3,4-d] pyrimidin-7(8H)-yl)naphthalen-2-ol: To a mixture of 7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (100 mg, 1.0 equiv), 2-(3-methyloxetan-3-yl)-2,4,5,6,7,8-hexahydropyrazolo[4,3-c]azepine (74.1 mg, 1.5 equiv, TFA salt) and 4 Å molecular sieve (50.0 mg, 1.0 equiv) in DMF (1 mL) was added DIEA (99.3 mg, 5.0 equiv). The reaction was stirred at 40° C. for 12 hours. The reaction was filtered and purified with prep-HPLC [Phenomenex C18 75×30 mm×3 m; A: water (FA), B: ACN, B %: 15%-45% over 7 min] to afford the title compound (37.3 mg, 35% yield, HCOOH salt) as yellow solid; ¹H NMR (400 MHz, METHANOL-d₄) δ=7.70 (s, 1H), 7.50 (dd, J=5.6, 8.8 Hz, 1H), 7.13 (t, J=9.6 Hz, 1H), 6.95 (s, 2H), 5.38-5.22 (m, 1H), 5.09-5.03 (m, 2H), 4.76 (br s, 2H), 4.63 (d, J=6.4 Hz, 2H), 4.23-3.99 (m, 5H), 3.65 (br d, J=17.6 Hz, 1H), 3.48 (br d, J=8.8 Hz, 1H), 3.35 (br d, J=8.8 Hz, 4H), 3.28 (br s, 1H), 3.23-3.12 (m, 2H), 3.11-3.03 (m, 1H), 3.02-2.88 (m, 2H), 2.73 (br d, J=13.2 Hz, 1H), 2.37-2.25 (m, 1H), 2.21 (br d, J=2.0 Hz, 1H), 2.13 (br d, J=9.2 Hz, 1H), 2.11-1.97 (m, 3H), 1.90 (br d, J=7.6 Hz, 2H), 1.85 (s, 3H), 1.08 (br t, J=6.8 Hz, 3H); LCMS (ESI, M+1): m/z=686.6.

### Example 494

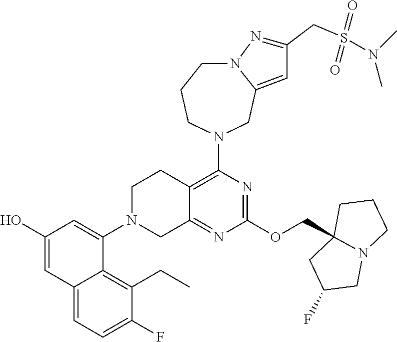 [SMILES: CCc1c(F)ccc2cc(O)cc(N3CCc4c(nc(OC[C@@]56CCCN5C[C@H](F)C6)nc4N4CCCn5nc(CS(=O)(=O)N(C)C)cc5C4)C3)c12]

### 1-(5-(7-(8-ethyl-7-fluoro-3-hydroxynaphthalen-1-yl)-2-(((2R,7aS)-2-fluorohexahydro-1H-pyrrolizin-7a-yl)methoxy)-5, 6,7, 8-tetrahydropyrido[3,4-d]pyrimidin-4-yl)-5, 6,7, 8-tetrahydro-4H-pyrazolo[1, 5-a][1,4]diazepin-2-yl)-N,N-dimethylmethanesulfonamide

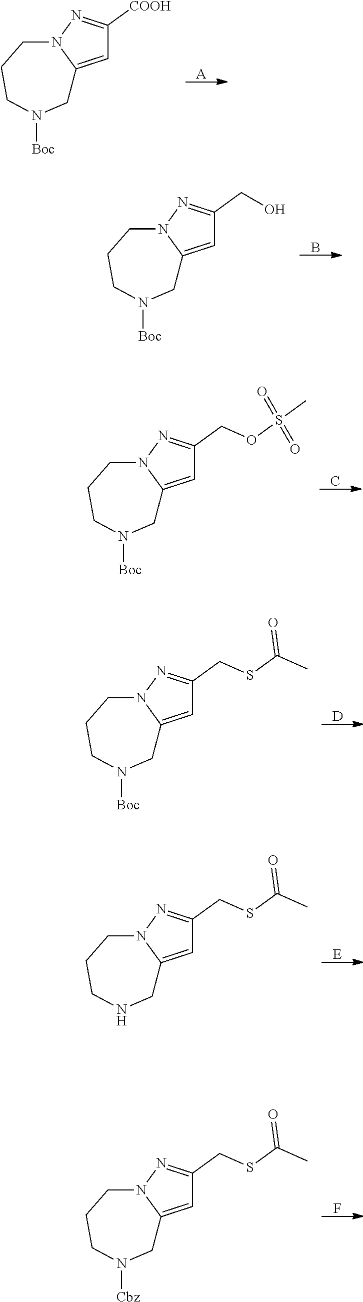

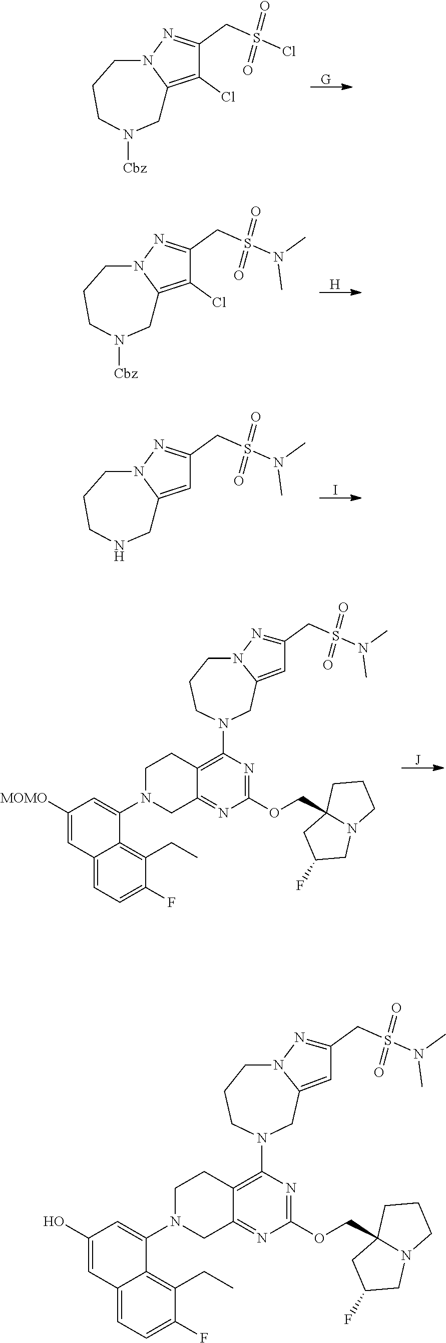

Step A. tert-butyl 2-(hydroxymethyl)-7,8-dihydro-4H-pyrazolo[1,5-a][1,4]diazepine-5(6H)-carboxylate: To a solution of 5-tert-butoxycarbonyl-4,6,7,8-tetrahydropyrazolo[1,5-a][1,4]diazepine-2-carboxylic acid (10.0 g, 1.0 equiv) in THe (100 mL) was added BH3-Me2S (10 M, 17.8 mL, 5.0 equiv) at 0° C. under N₂ atmosphere. The reaction was stirred at 20° C. for 0.5 hours and at 60° C. for another 2 hours. The mixture was quenched with methanol (40 mL) at 0° C. and stirred at 20° C. for 1 hour under N₂ atmosphere. The mixture was concentrated, diluted with water (20 mL) and extracted with ethyl acetate (3×20 mL). The combined organic layers were washed with brine (10 mL), dried over sodium sulfate, and concentrated to afford the title compound (8.50 g, 85% yield) as white solid; LCMS (ESI, M+1): m/z=268.1.

Step B. tert-butyl 2-(((methylsulfonyl)oxy)methyl)-7,8-dihydro-4H-pyrazolo[1,5-a][1,4]diazepine-5(6H)-carboxylate To a solution of tert-butyl 2-(hydroxymethyl)-7,8-dihydro-4H-pyrazolo[1,5-a][1,4]diazepine-5(6H)-carboxylate (8.50 g, 1.0 equiv) and TEA (9.65 g, 3.0 equiv) in dichloromethane (80 mL) was added MsCl (5.61 g, 1.5 equiv) at 0° C. under N₂ atmosphere. The reaction was degassed and purged with N₂ for 3 times and stirred at 20° C. for 0.5 hours under N₂ atmosphere. The mixture was diluted with ice-water (30 mL) and extracted with dichloromethane (3×30 mL). The combined organic layers were washed with brine (30 mL), dried over sodium sulfate, and concentrated to afford the title compound (3.78 g, 19% yield) as colorless liquid.

Step C. tert-butyl 2-((acetylthio)methyl)-7,8-dihydro-4H-pyrazolo[1,5-a][1,4]diazepine-5(6H)-carboxylate: To a solution of tert-butyl 2-(((methylsulfonyl)oxy)methyl)-7,8-dihydro-4H-pyrazolo[1,5-a][1,4]diazepine-5(6H)-carboxylate (3.78 g, 1.0 equiv) in DMF (50 mL) was added acetylsulfanylpotassium (6.60 g, 5.3 equiv). The reaction was stirred at 95° C. for 2 hours. The mixture was diluted with water (20 mL) and extracted with ethyl acetate (4×20 mL). The organic layers were washed with brine (3×20 mL), dried over sodium sulfate, concentrated, and purified by column chromatography (SiO₂, Petroleum ether/Ethyl acetate=10/1 to 2/1) to afford the title compound (3 g, 76% yield) as brown liquid; LCMS (ESI, M+1): m/z=326.2

Step D. S-((5,6,7,8-tetrahydro-4H-pyrazolo[1,5-a][1,4]diazepin-2-yl)methyl) ethanethioate: To a solution of tert-butyl 2-((acetylthio)methyl)-7,8-dihydro-4H-pyrazolo[1,5-a][1,4]diazepine-5(6H)-carboxylate (2.50 g, 1.0 equiv) in ACN (20 mL) was added HCl/dioxane (4 M, 20 mL, 10 equiv). The reaction was stirred at 20° C. for 0.5 hour. The mixture was concentrated to afford the title compound (2.00 g, crude) as brown liquid.

Step E. benzyl 2-((acetylthio)methyl)-7,8-dihydro-4H-pyrazolo[1,5-a][1,4]diazepine-5(6H)-carboxylate: To a mixture of S-((5,6,7,8-tetrahydro-4H-pyrazolo[1,5-a][1,4]diazepin-2-yl)methyl) ethanethioate (2.00 g, 1.0 equiv, HCl) and NaHCO₃ (4.52 g, 7.0 equiv) in EtOAc (12 mL) and water (8 mL) was added benzyl carbonochloridate (1.70 g, 1.3 equiv) at 0° C. The reaction was stirred at 20° C. for 12 hours. The mixture was extracted with EtOAc (10 mL×3). The combined organic layers were washed with brine (20 mL), dried over sodium sulfate, and concentrated to afford the title compound (3.00 g, crude) as brown liquid; LCMS (ESI, M+1): m/z=360.2

Step F. benzyl 3-chloro-2-((chlorosulfonyll)methyl)-7,8-dihydro-4H-pyrazolo[1,5-a]l[1,4]diazepine-5(6H)-carboxylate: To a solution of NCS (2.23 g, 3.0 equiv) and HCl (3.0 M, 984 mg, 4.8 equiv) in ACN (20 mL) was added benzyl 2-((acetylthio)methyl)-7,8-dihydro-4H-pyrazolo[1,5-a][1,4]diazepine-5(6H)-carboxylate (2.00 g, 1.0 equiv). The reaction was stirred at 10˜20° C. for 2 hours. The mixture was extracted with DCM (10 mL×3). The combined organic layers were washed with brine (15 mL), dried over sodium sulfate, and concentrated to afford the title compound (2.10 g, crude) as white solid; LCMS (ESI, M+1, M+3): m/z=418.1, 420.1

Step G. benzyl 3-chloro-2-((N,N-dimethylsulfamoyl)methyl)-7,8-dihydro-4H-pyrazolo[1,5-a][1,4]diazepine-5(6H)-carboxylate: To a solution of benzyl 3-chloro-2-((chlorosulfonyl)methyl)-7,8-dihydro-4H-pyrazolo[1,5-a][1,4]diazepine-5(6H)-carboxylate (1.00 g, 1.0 equiv) in DCM (10 mL) was added N-methylmethanamine (2 M, 992 mg, 9.2 equiv). The mixture was stirred at 0-20° C. for 3 hours. The mixture was filtered and purified by reversed phase chromatography (C 18, A: water [(0.1% FA)-ACN]; B %: 45%-65% over 25 min) to afford the title compound (300 mg, 24% yield) as brown solid; LCMS (ESI, M+1): m/z=427.1

Step H. N,N-dimethyl-1-(5,6,7,8-tetrahydro-4H-pyrazolo[1,5-a][1,4]diazepin-2-yl)methanesulfonamide: To a solution of benzyl 3-chloro-2-((N,N-dimethylsulfamoyl)methyl)-7,8-dihydro-4H-pyrazolo[1,5-a][1,4]diazepine-5(6H)-carboxylate (150 mg, 1.0 equiv) in isopropanol (3 mL) was added Pd/C (15.0 mg, 10% purity) under N₂ atmosphere. The suspension was degassed and purged with N₂ and then H₂ for 3 times. Then the reaction was stirred at 40° C. for 1 hour under H₂ (15 Psi). The mixture was filtered and concentrated to afford the title compound (80.0 mg, 65% yield) as yellow solid.

The last two steps were performed according to Example 248. The title compound was obtained as off-white solid (FA salt). ¹H NMR (400 MHz, DMSO-d₆) δ=9.77-9.64 (m, 1H), 8.26 (s, 1H), 7.60 (dd, J=6.0, 9.2 Hz, 1H), 7.25 (t, J=9.6 Hz, 1H), 6.99 (d, J=2.4 Hz, 1H), 6.97 (s, 1H), 6.20 (d, J=1.2 Hz, 1H), 5.34-5.15 (m, 1H), 4.98-4.90 (m, 1H), 4.74 (br dd, J=2.8, 16.4 Hz, 1H), 4.47 (br d, J=4.4 Hz, 2H), 4.34-4.23 (m, 2H), 4.12-4.01 (m, 1H), 3.99-3.76 (m, 5H), 3.61 (br d, J=17.6 Hz, 2H), 3.11 (br d, J=8.4 Hz, 2H), 3.06 (br d, J=3.6 Hz, 2H), 2.98 (br s, 1H), 2.84-2.77 (m, 1H), 2.62 (br s, 2H), 2.58 (d, J=1.6 Hz, 6H), 2.19-2.10 (m, 1H), 2.08-2.03 (m, 1H), 2.00-1.90 (m, 3H), 1.85-1.70 (m, 3H), 1.08 (br t, J=7.2 Hz, 3H); LCMS (ESI, M+1): m/z=737.3.

### Example 495

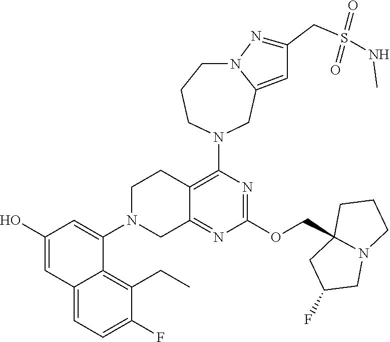 [SMILES: CCc1c(F)ccc2cc(O)cc(N3CCc4c(nc(OC[C@@]56CCCN5C[C@H](F)C6)nc4N4CCCn5nc(CS(=O)(=O)NC)cc5C4)C3)c12]

### 1-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)-N-methylmethanesulfonamide

Synthesized according to Example 494. The title compound was obtained as off-white solid (FA salt). ¹H NMR (400 MHz, DMSO-d₆) δ=9.68 (br s, 1H), 8.17-8.14 (m, 1H), 7.59 (dd, J=6.0, 9.2 Hz, 1H), 7.24 (t, J=9.6 Hz, 1H), 6.98 (d, J=2.4 Hz, 1H), 6.96 (d, J=2.4 Hz, 1H), 6.93-6.88 (m, 1H), 6.19 (s, 1H), 5.34-5.16 (m, 1H), 4.95 (br d, J=16 Hz, 1H), 4.72 (br d, J=16.4 Hz, 1H), 4.44 (br t, J=5.2 Hz, 2H), 4.23-4.13 (m, 2H), 4.12-4.04 (m, 1H), 3.95-3.90 (m, 1H), 3.90-3.87 (m, 1H), 3.85 (br s, 1H), 3.82-3.77 (m, 1H), 3.62 (br d, J=17.2 Hz, 1H), 3.40 (br s, 3H), 3.11-3.04 (m, 4H), 2.99 (br s, 1H), 2.85-2.77 (m, 1H), 2.63 (br d, J=9.6 Hz, 1H), 2.49-2.48 (m, 3H), 2.20-2.10 (m, 1H), 2.07 (br d, J=4.0 Hz, 1H), 2.00-1.90 (m, 3H), 1.85-1.68 (m, 3H), 1.07 (t, J=7.2 Hz, 3H). LCMS (ESI, M+1): m/z=723.2.

### Example 496

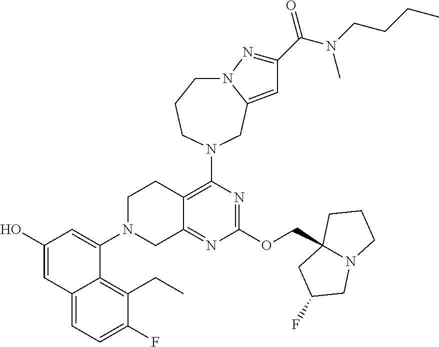 [SMILES: CCCCN(C)C(=O)c1cc2n(n1)CCCN(c1nc(OC[C@@]34CCCN3C[C@H](F)C4)nc3c1CCN(c1cc(O)cc4ccc(F)c(CC)c14)C3)C2]

### N-butyl-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepine-2-carboxamide

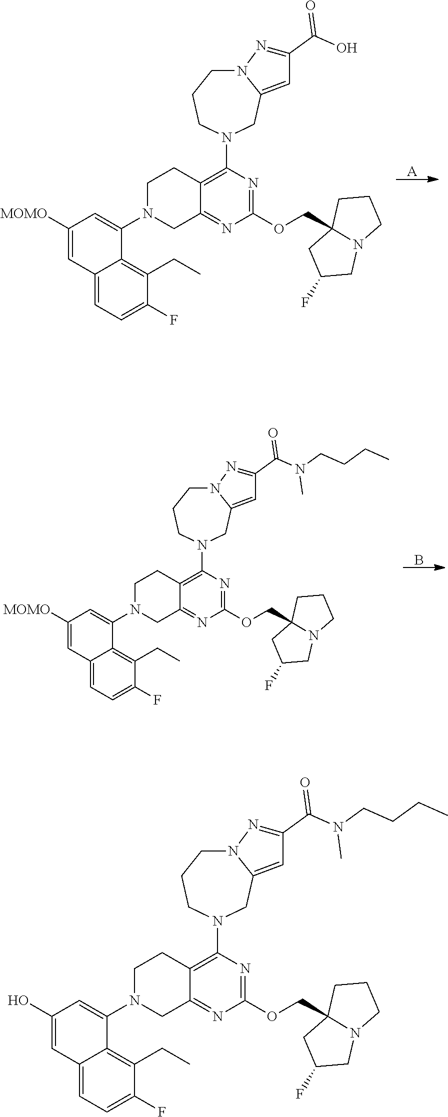

Synthesized according to Example 233 except that HCl instead of TFA was used in the last step. The title compound was obtained as off-white solid (FA salt). ¹H NMR (400 MHz, METHANOL-d₄) δ=7.53 (dd, J=6.0, 8.8 Hz, 1H), 7.16 (t, J=9.2 Hz, 1H), 6.99 (s, 2H), 6.68-6.56 (m, 1H), 5.45-5.21 (m, 1H), 5.07-4.98 (m, 1H), 4.84 (br d, J=3.6 Hz, 1H), 4.62-4.50 (m, 2H), 4.28-3.97 (m, 5H), 3.81-3.64 (m, 2H), 3.59-3.50 (m, 2H), 3.37 (br d, J=13.2 Hz, 4H), 3.30 (s, 5H), 3.11-3.05 (m, 2H), 2.75 (br d, J=14.4 Hz, 1H), 2.41-2.22 (m, 3H), 2.21-2.00 (m, 4H), 1.99-1.84 (m, 1H), 1.72-1.53 (m, 2H), 1.47-1.34 (m, 1H), 1.33-1.20 (m, 1H), 1.13 (br s, 3H), 1.05-0.79 (m, 3H); LCMS (ESI, M+1): m/z=729.7.

### Example 497

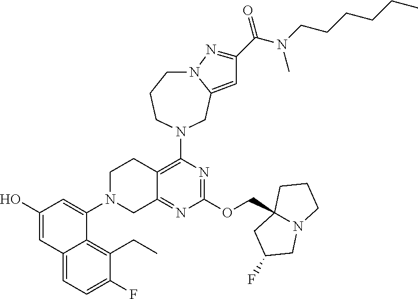 [SMILES: CCCCCCN(C)C(=O)c1cc2n(n1)CCCN(c1nc(OC[C@@]34CCCN3C[C@H](F)C4)nc3c1CCN(c1cc(O)cc4ccc(F)c(CC)c14)C3)C2]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N-hexyl-N-methyl-5,6,7,8-tetrahydro-4H-pyrazolo[1,5-a][1,4]diazepine-2-carboxamide

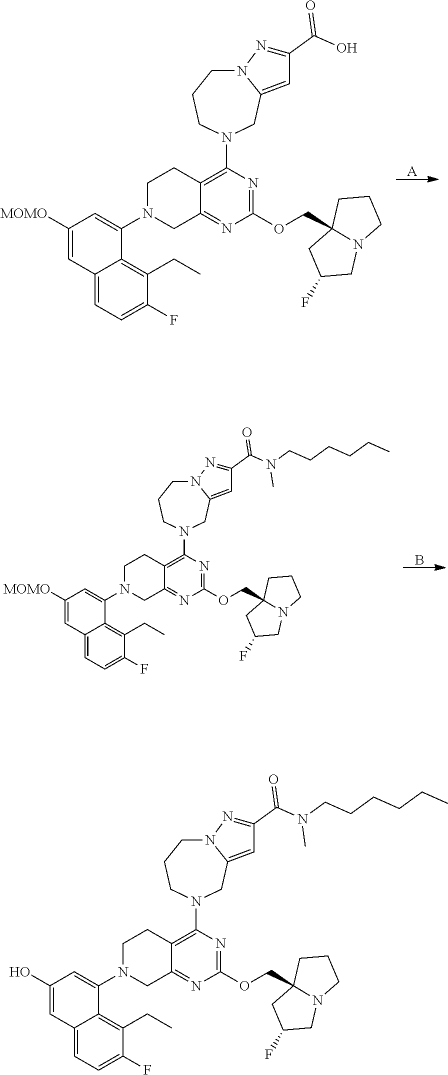

Synthesized according to Example 233 except that HCl instead of TFA was used in the last step. The title compound was obtained as pink solid (FA salt). ¹H NMR (400 MHz, METHANOL-d₄) δ=7.51 (dd, J=6.0, 9.2 Hz, 1H), 7.14 (t, J=9.2 Hz, 1H), 7,02-6.92 (m, 2H), 6.66-6.52 (m, 1H), 5.42-5.14 (m, 1H), 5.08-4.94 (m, 1H), 4.81 (br d, J=5.2 Hz, 1H), 4.62-4.45 (m, 2H), 4.29-3.93 (m, 5H), 3.78-3.60 (m, 2H), 3.57-3.45 (m, 2H), 3.44-3.34 (m, 2H), 3.29-3.20 (m, 4H), 3.20-3.12 (m, 2H), 3.09-2.89 (m, 3H), 2.72 (br d, J=14.4 Hz, 1H), 2.37-2.13 (m, 3H), 2.12-2.02 (m, 2H), 2.01-1.91 (m, 2H), 1.86 (br dd, J=1.6, 4.8 Hz, 1H), 1.63 (br d, J=6.0 Hz, 2H), 1.36 (br s, 3H), 1.22 (br s, 3H), 1.11 (br t, J=6.8 Hz, 3H), 0.99-0.73 (m, 3H); LCMS (ESI, M+1): m/z=757.5.

### Example 498

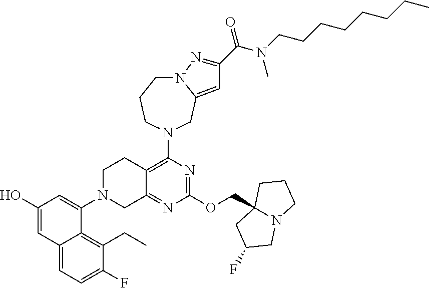 [SMILES: CCCCCCCCN(C)C(=O)c1cc2n(n1)CCCN(c1nc(OC[C@@]34CCCN3C[C@H](F)C4)nc3c1CCN(c1cc(O)cc4ccc(F)c(CC)c14)C3)C2]

### 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N-methyl-N-octyl-5,6,7,8-tetrahydro-4H-pyrazolo[1,5-a][1,4]diazepine-2-carboxamide

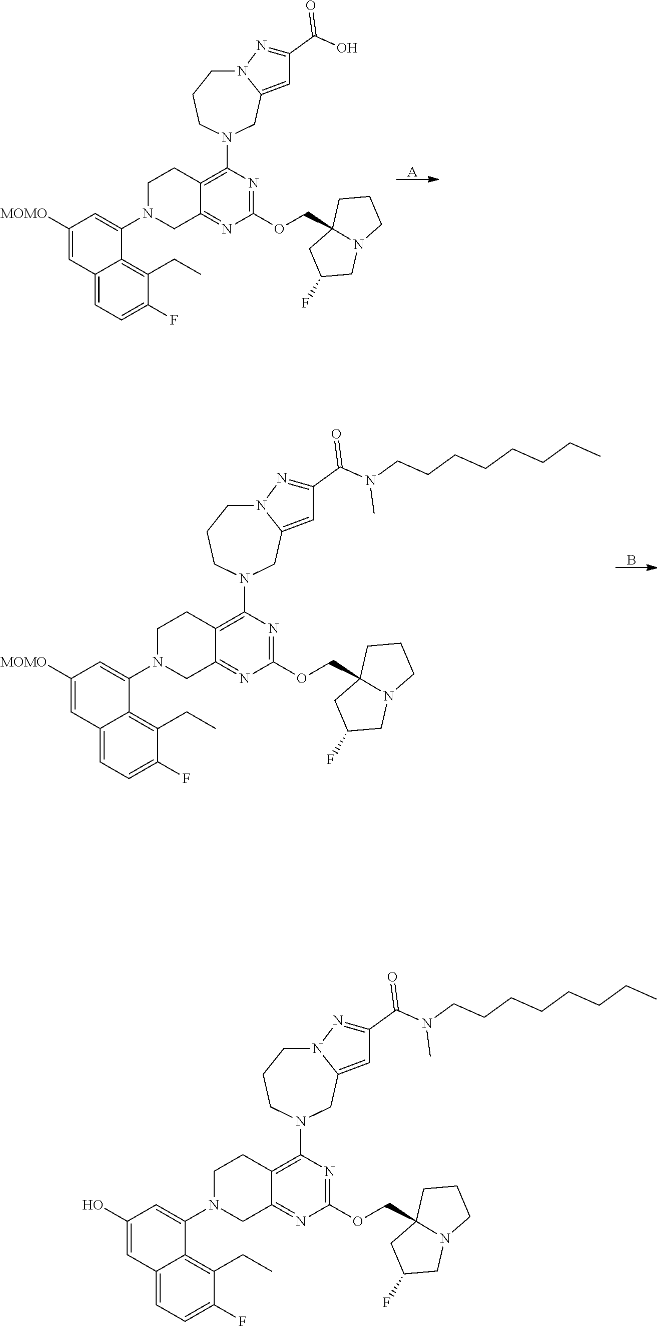

Synthesized according to Example 233 except that HCl instead of TFA was used in the last step. The title compound was obtained as off-white solid (FA salt). ¹H NMR (400 MHz, METHANOL-d₄) δ=7.51 (dd, J=5.6, 8.9 Hz, 1H), 7.14 (t, J=9.2 Hz, 1H), 6.97 (s, 2H), 6.63-6.52 (m, 1H), 5.39-5.18 (m, 1H), 5.06-4.93 (m, 1H), 4.82-4.80 (m, 1H), 4.60-4.51 (m, 2H), 4.24-3.97 (m, 5H), 3.76-3.61 (m, 2H), 3.58-3.45 (m, 2H), 3.43-3.34 (m, 2H), 3.29-3.16 (m, 6H), 3.10-2.95 (m, 3H), 2.73 (br d, J=13.6 Hz, 1H), 2.37-2.22 (m, 2H), 2.21-2.15 (m, 1H), 2.13-1.94 (m, 4H), 1.88 (br d, J=6.4 Hz, 1H), 1.63 (br d, J=5.2 Hz, 2H), 1.40-1.28 (m, 5H), 1.24 (br s, 5H), 1.10 (br d, J=4.4 Hz, 3H), 0.92-0.83 (m, 3H); LCMS (ESI, M+1). m/z=785.5.

### Example 499

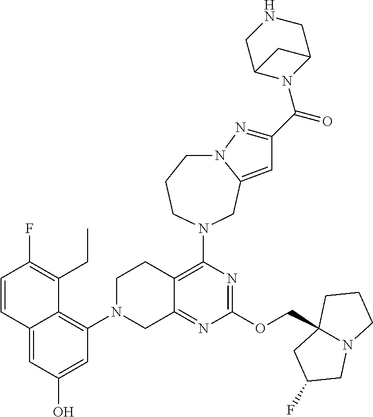 [SMILES: CCc1c(F)ccc2cc(O)cc(N3CCc4c(nc(OC[C@@]56CCCN5C[C@H](F)C6)nc4N4CCCn5nc(C(=O)N6C7CNCC6C7)cc5C4)C3)c12]

### (1R,5S)-3, 6-diazabicyclo[3.1.1]heptan-6-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

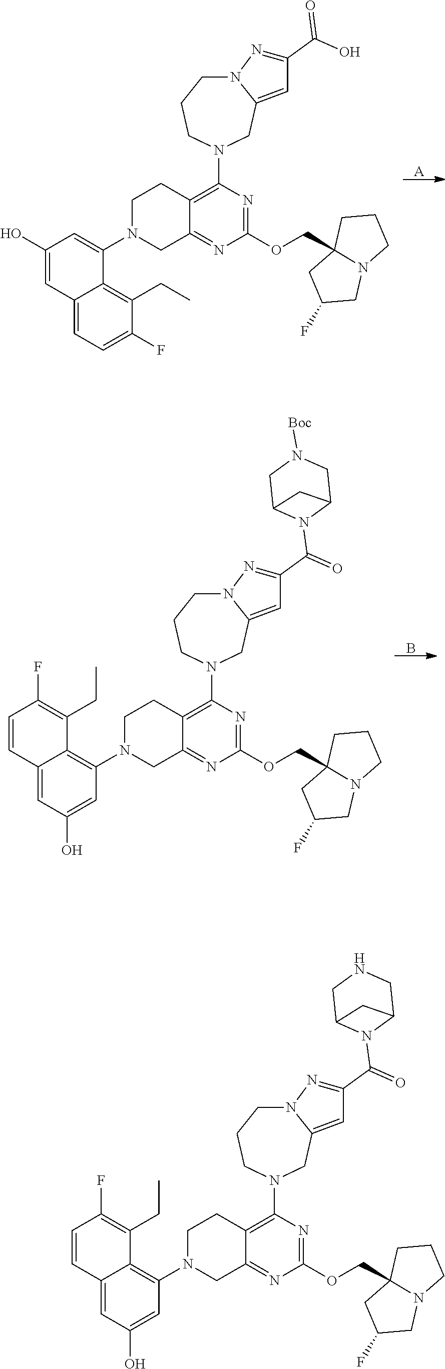

Step A. (1R,5S)-tert-butyl 6-(5-7-(8-ethyl-7-fluoro-3-hy droxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7, 8-tetrahydro-4H-pyrazolo[1,5-a][1,4]diazepine-2-carbonyl)-3,6-diazabicyclo[3.1.1]heptane-3-carboxylate: To a mixture of 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepine-2-carboxylic acid (55.0 mg, 1.0 equiv) and tert-butyl 3,6-diazabicyclo[3.1.1]heptane-3-carboxyl ate (23.0 mg, 1.4 equiv) in DMF (1.0 mL) were added EDCI (32.0 mg, 2.0 equiv) and HOBt (33.8 mg, 3.0 equiv). The reaction was stirred at 25° C. for 0.5 hour. The mixture was filtered and purified by prep-HPLC [Phenomenex luna C18 150×25 mm×10 μm; A: water (FA), B: ACN; B %:30%-60% over 10 min] to afford the title compound (44.0 mg, 63% yield) as yellow solid, LCMS (ESI, M+1): m/z=840.7.

Step B. (1R,5S)-3,6-diazabicyclo[3.1.1]heptan-6-vl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone: To a mixture of (1R,5S)-tert-butyl 6-(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepine-2-carbonyl)-3,6-diazabicyclo[3.1.1]heptane-3-carboxylate (40.0 mg, 1.0 equiv) and DCM (0.5 mL) was added TFA (770 mg, 142 equiv). The reaction was stirred at 0° C. for 1 hour. The mixture was concentrated, dissolved in methanol (3.0 mL), neutralized with solid NaHCO₃, filtered, and purified with prep-HPLC [Phenomenex luna C18 150×25 mm×10 μm; A: water (FA), B: ACN; B %: 7%-37% over 9 min] to afford the title compound (13.4 mg, 37% yield, HCOOH salt) as off-white solid. ¹H NMR (400 MHz, dimethylsulfoxide-d₆+deuterium oxide-d₂) δ=7.56 (dd, J=6.0, 8.8 Hz, 1H), 7.22 (t, J=9.6 Hz, 1H), 6.98 (s, 2H), 6.72-6.61 (m, 1H), 5.39-5.18 (m, 1H), 5.09-4.96 (m, 1H), 4.93 (br s, 1H), 4.81-4.68 (m, 1H), 4.47 (br s, 2H), 4.41 (br s, 1H), 4.28-4.11 (m, 1H), 4.01 (br d, J=10.4 Hz, 1H), 3.93 (br s, 1H), 3.76 (br s, 1H), 3.62-3.51 (m, 1H), 3.50-3.37 (m, 3H), 3.33-3.13 (m, 7H), 3.13-3.01 (m, 2H), 2.86 (br s, 1H), 2.74-2.59 (m, 2H), 2.23-2.10 (m, 2H), 2.09-1.86 (m, 4H), 1.86-1.63 (m, 4H), 1.04 (q, J=7.6 Hz, 3H); ¹⁹F NMR (400 MHz, dimethylsulfoxide-d₆+deuterium oxide-d₂) δ=−120.988, -171.806; LCMS (ESI, M+1): m/z=740.5.

### Example 500

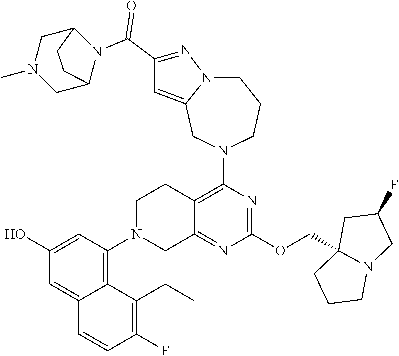 [SMILES: CCc1c(F)ccc2cc(O)cc(N3CCc4c(nc(OC[C@@]56CCCN5C[C@H](F)C6)nc4N4CCCn5nc(C(=O)N6C7CCC6CN(C)C7)cc5C4)C3)c12]

### (5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(6-methyl-3,6-diazabicyclo[3.2.1]octan-3-yl)methanone

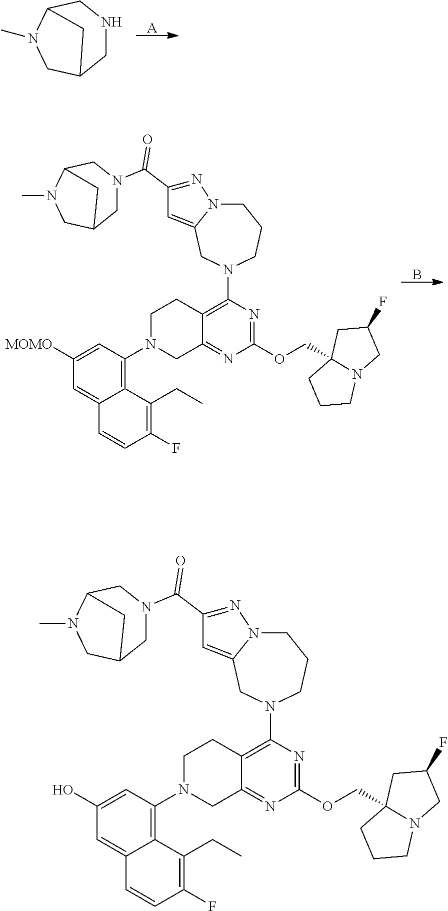

Synthesized according to Example 233 except that HCl instead of TFA was used in the last step. The title compound was obtained as yellow solid (FA salt). ¹H NMR (400 MHz, dimethylsulfoxide-d₆+deuterium oxide-d₂) δ=7.56 (dd, J=6.0, 9.2 Hz, 1H), 7.22 (t, J=9.6 Hz, 1H), 6.97 (br d, J=2.4 Hz, 2H), 6.56 (s, 1H), 5.40-5.18 (m, 1H), 4.99 (br d, J=16.0 Hz, 1H), 4.73 (br d, J=16.4 Hz, 1H), 4.46 (br s, 3H), 4.27-4.02 (m, 2H), 4.01-3.96 (m, 1H), 3.82 (br d, J=17.6 Hz, 2H), 3.55 (br d, J=17.6 Hz, 2H), 3.41 (br d, J=6.4 Hz, 1H), 3.27-3.12 (m, 8H), 3.10-2.97 (m, 2H), 2.96-2.84 (m, 2H), 2.68-2.57 (m, 5H), 2.15 (br s, 2H), 2.07 (br s, 2H), 2.06-1.88 (m, 4H), 1.87-1.68 (m, 3H), 1.02 (br t, J=6.8 Hz, 3H); ¹⁹F NMR (400 MHz, dimethylsulfoxide-d₆+deuterium oxide-d₂) δ=−120.978, -171.810; LCMS (ESI, M+1): m/z=768.6.

### Example 501

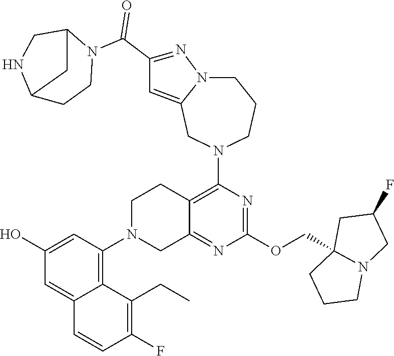 [SMILES: CCc1c(F)ccc2cc(O)cc(N3CCc4c(nc(OC[C@@]56CCCN5C[C@H](F)C6)nc4N4CCCn5nc(C(=O)N6CCC7CC6CN7)cc5C4)C3)c12]

### (5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(6-methyl-2,6-diazabicyclo[3.2.1]octan-2-yl)methanone

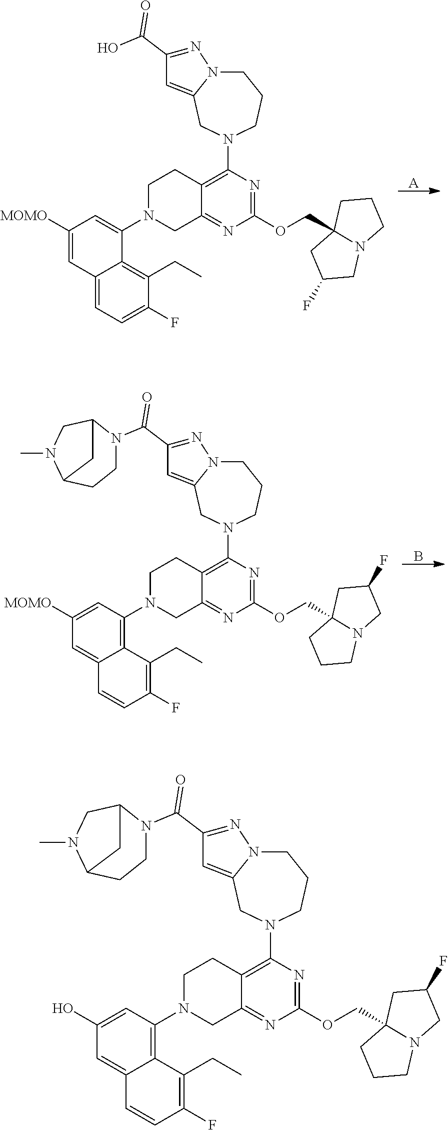

Synthesized according to Example 233 except that HCl instead of TFA was used in the last step. The title compound was obtained as off-white solid (FA salt). ¹H NMR (400 MHz, dimethylsulfoxide-d₆) δ=7.59 (dd, J=6.0, 8.8 Hz, 1H), 7.24 (t, J=9.2 Hz, 1H), 6.99 (s, 2H), 6.48 (br t, J=5.6 Hz, 1H), 5.40-4.92 (m, 3H), 4.83-4.69 (m, 1H), 4.65-4.25 (m, 3H), 4.24-4.02 (m, 2H), 3.99-3.72 (m, 5H), 3.66-3.55 (m, 2H), 3.18-3.12 (m, 2H), 3.11-3.02 (m, 3H), 3.01-2.87 (m, 2H), 2.86-2.69 (m, 3H), 2.68-2.60 (m, 1H), 2.39 (br d, J=6.4 Hz, 3H), 2.28-2.08 (m, 2H), 2.08-2.02 (m, 1H), 2.01-1.87 (m, 3H), 1.85-1.68 (m, 4H), 1.66-1.51 (m, 1H), 1.50-1.36 (m, 1H), 1.11-1.00 (m, 3H) 19F NMR (400 MHz, dimethylsulfoxide-d₆) δ=−121.371, -171.977; LCMS (ESI, M+1): m/z=768.6.

### Example 502

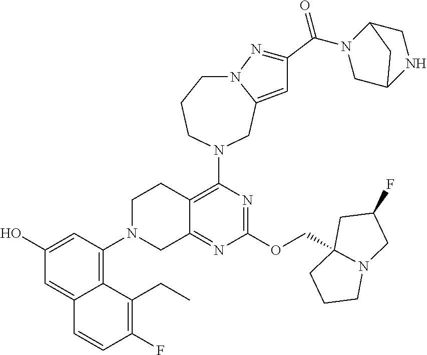 [SMILES: CCc1c(F)ccc2cc(O)cc(N3CCc4c(nc(OC[C@@]56CCCN5C[C@H](F)C6)nc4N4CCCn5nc(C(=O)N6CC7CC6CN7)cc5C4)C3)c12]

### 2,5-diazabicyclo[2.2.1]heptan-2-yl(5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

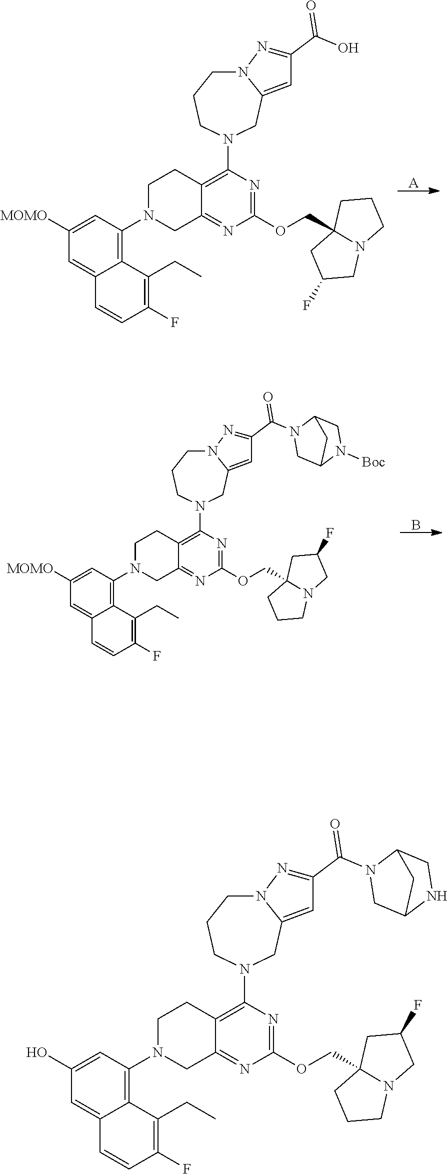

Synthesized according to Example 233 except that HCl instead of TFA was used in the last step. The title compound was obtained as yellow solid (FA salt). ¹H NMR (400 MHz, dimethylsulfoxide-d₆+deuterium oxide-d₂) δ=7.61-7.52 (m, 1H), 7.27-7.18 (m, 1H), 7.02-6.94 (m, 2H), 6.71-6.61 (m, 1H), 5.68-5.54 (m, 0.5H), 5.39-5.18 (m, 1H), 5.07-4.92 (m, 1H), 4.88-4.83 (m, 0.5H), 4.83-4.66 (m, 1H), 4.58-4.42 (m, 2H), 4.39-4.29 (m, 1H), 4.27-4.11 (m, 1H), 4.07-3.94 (m, 3H), 3.84-3.75 (m, 2H), 3.61-3.48 (m, 2H), 3.47-3.37 (m, 1H), 3.36-3.26 (m, 1H), 3.26-3.10 (m, 7H), 3.10-3.00 (m, 1H), 2.93-2.81 (m, 1H), 2.71-2.59 (m, 1H), 2.22-2.10 (m, 2H), 2.10-2.04 (m, 1H), 2.03-1.67 (m, 7H), 1.11-0.95 (m, 3H); ¹⁹F NMR (400 MHz, dimethylsulfoxide-d₆+deuterium oxide-d₂) δ=−120.951, -171.836; LCMS (ESI, M+1): m/z=740.7.

### Example 503

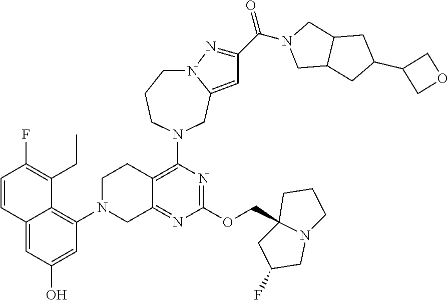 [SMILES: CCc1c(F)ccc2cc(O)cc(N3CCc4c(nc(OC[C@@]56CCCN5C[C@H](F)C6)nc4N4CCCn5nc(C(=O)N6CC7CC(C8COC8)CC7C6)cc5C4)C3)c12]

### (5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(5-(oxetan-3-yl)hexahydropyrrolo[3,4-c]pyrrol

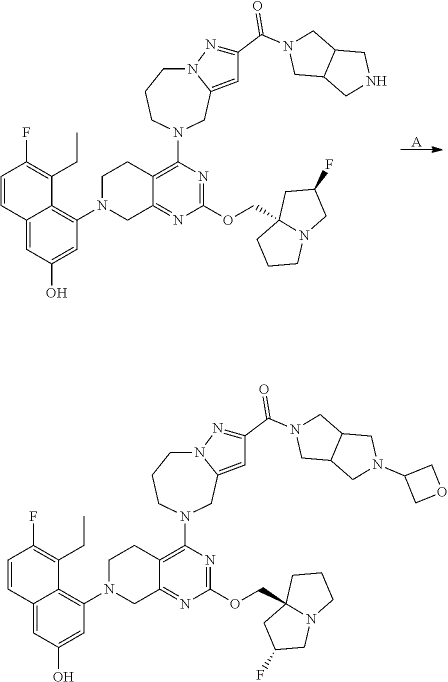

Step A. (5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d] pyrimidin-4-yl)-⁵,6,7,8-tetrahydro-4H-pyrazolo[1,5-a][1,4]diazepin-2-yl)(5-(oxetan-3-yl)hexahydropyrrolo[3,4-clpyrrol-2(1H)-yl)methanone: To a solution of (5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(hexahydropyrrolo[3,4-c]pyrrol-2(1H)-yl)methanone (9.00 mg, 1.0 equiv) and oxetan-3-one (2.58 mg, 3.0 equiv) in dichlomethane (2 mL) was added dropwise HOAc (7.17 mg, 10 equiv) at 25° C. The mixture was stirred at this temperature for 1 hours. NaBH(OAc)₃ (7.59 mg, 3.0 equiv) was added in portions at 25° C. The resulting mixture was stirred at 25° C. for 2 hours. The mixture was diluted with water (40 mL) and extracted with ethyl acetate (2×30 mL). The combined layers were washed with brine (40 mL), dried over sodium sulfate, concentrated and purified with prep-HPLC [column: Phenomenex luna C18 150×25 mm×10 μm; mobile phase: water(FA)-ACN]; B %: 7%-37%,10 minutes], and re-purified by prep-HPLC [column: Waters Xbridge 150×25 mm×5 μm; mobile phase: [water (ammonia hydroxide v/v)-ACN];B %: 37%-67%, 9 minutes] to afford the title compound (5.00 mg) as white solid. ¹H NMR (400 MHz, DMSO-d₆) 5=9.74 (s, 1H), 7.60 (dd, J=6.0, 8.8 Hz, 1H), 7.40 (d, J=4.0 Hz, 1H), 7.25 (br t, J=9.6 Hz, 1H), 7.00 (s, 2H), 6.67 (br s, 1H), 5.69-5.41 (m, 1H), 5.05-4.77 (m, 2H), 4.65-4.39 (m, 6H), 4.36-4.04 (m, 4H), 4.00-3.57 (m, 10H), 3.51-3.42 (m, 2H), 3.18-3.03 (m, 2H), 2.84-2.62 (m, 3H), 2.37-1.84 (m, 12H), 1.23 (s, 1H), 1.07 (br t, J=7.2 Hz, 3H). LCMS (ESI, M+1): m/z=810.6.

### Example 504

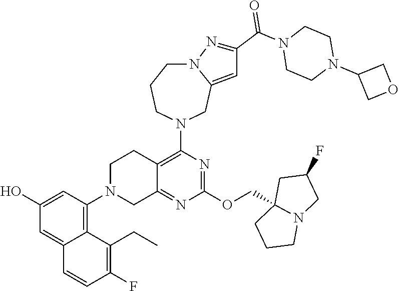 [SMILES: CCc1c(F)ccc2cc(O)cc(N3CCc4c(nc(OC[C@@]56CCCN5C[C@H](F)C6)nc4N4CCCn5nc(C(=O)N6CCN(C7COC7)CC6)cc5C4)C3)c12]

### (5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(4-(oxetan-3-yl)piperazin-1-yl)methanone

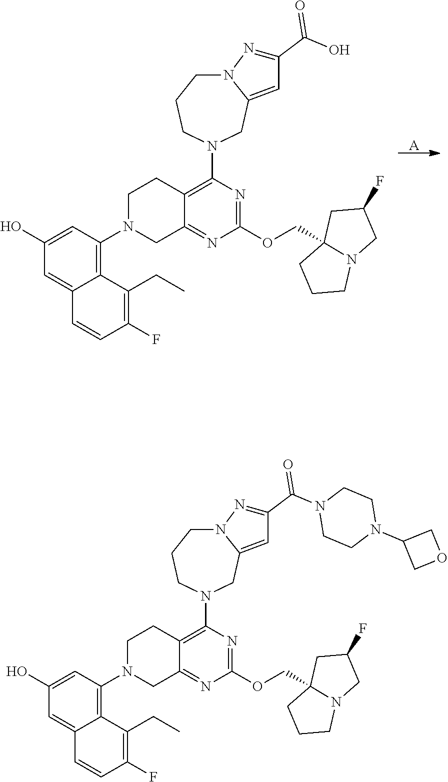

Step A. (5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(4-(oxetan-3-yl)piperazin-1-l)methanone: To a solution of 5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepine-2-carboxylic acid (20.0 mg, 1.0 equiv), TEA (9.20 mg, 3.0 equiv) and 1-(oxetan-3-yl) piperazine (6.47 mg, 1.5 equiv) in DMF (0.5 mL) was added EDCI (8.72 mg, 1.5 equiv) and HOBt (2.05 mg, 0.5 equiv). The reaction was stirred at 25° C. for 1 hour. The mixture was filtered, concentrated and purified by prep-HPLC [Phenomenex Luna C18 150×25 mm×10 μm; A: water (NH₄HCO₃), B:ACN; B %: 37%-67% over 8 min] to afford the title compound (1.89 mg, 7.4% yield) as white solid. ¹H NMR (400 MHz, dimethylsulfoxide-d₆) δ=9.68 (s, 1H), 7.60 (dd, J=6.0, 9.6 Hz, 1H), 7.25 (t, J=9.6 Hz, 1H), 6.99 (s, 2H), 6.55-6.50 (m, 1H), 5.33-5.13 (m, 1H), 5.01 (br d, J=16.0 Hz, 1H), 4.79-4.69 (m, 1H), 4.57-4.40 (m, 6H), 4.22-4.10 (m, 1H), 3.99-3.75 (m, 6H), 3.68-3.52 (m, 3H), 3.48-3.34 (m, 2H), 3.30-3.11 (m, 5H), 3.10-3.01 (m, 3H), 2.99-2.95 (m, 1H), 2.83-2.76 (m, 1H), 2.70-2.65 (m, 1H), 2.26 (br s, 3H), 2.05 (br dd, J=2.4, 9.6 Hz, 1H), 2.01-1.96 (m, 2H), 1.94-1.90 (m, 1H), 1.85-1.79 (m, 1H), 1.76-1.68 (m, 2H), 1.10-1.02 (m, 3H); ¹⁹F NMR (400 MHz, dimethylsulfoxide-d₆) δ=−121.341, -171.994; LCMS (ESI, M+1): m/z=784.6.

### Example 505

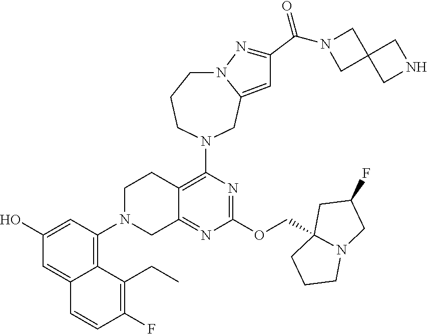 [SMILES: CCc1c(F)ccc2cc(O)cc(N3CCc4c(nc(OC[C@@]56CCCN5C[C@H](F)C6)nc4N4CCCn5nc(C(=O)N6CC7(CNC7)C6)cc5C4)C3)c12]

### (5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(2,6-diazaspiro[3.3]heptan-2-yl)methanone

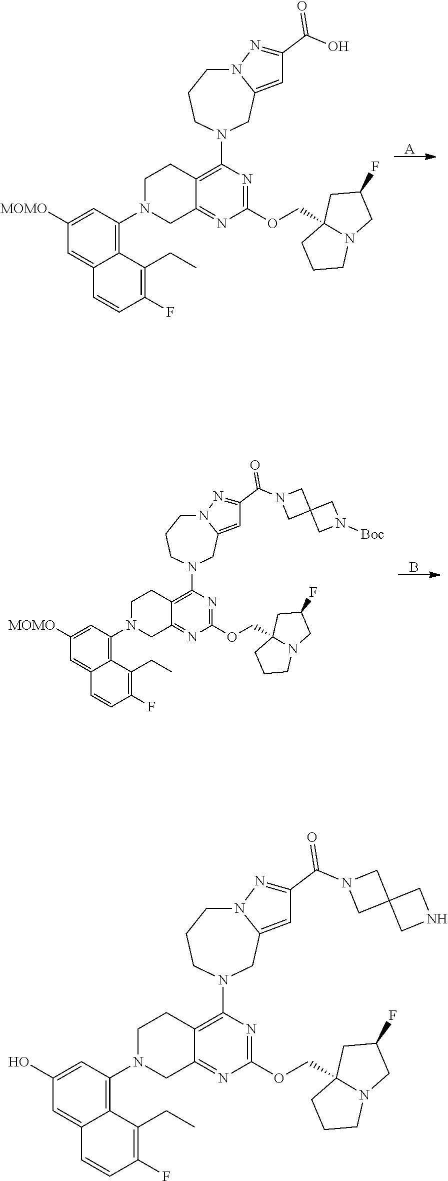

Synthesized according to Example 233. The title compound was obtained as yellow solid (FA salt). ¹H NMR (400 MHz, methanol-d₄) δ=7.51 (dd, J=5.7, 9.2 Hz, 1H), 7.14 (t, J=9.6 Hz, 1H), 6.99-6.96 (m, 2H), 6.69 (s, 1H), 5.38-5.14 (m, 3H), 5.06-4.93 (m, 2H), 4.59-4.52 (m, 4H), 4.30 (s, 2H), 4.15-3.94 (m, 6H), 3.58-3.38 (m, 5H), 3.27-3.18 (m, 5H), 3.05-2.98 (m, 1H), 2.77-2.70 (m, 1H), 2.12-1.94 (m, 8H), 1.14-1.09 (m, 3H); LCMS (ESI, M+1): m/z=740.5.

### Example 506

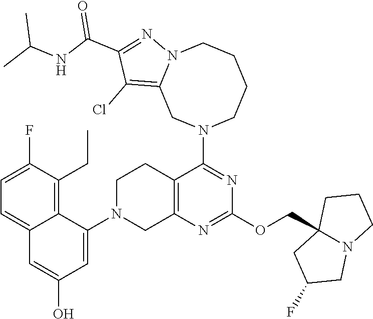 [SMILES: CCc1c(F)ccc2cc(O)cc(N3CCc4c(nc(OC[C@@]56CCCN5C[C@H](F)C6)nc4N4CCCCn5nc(C(=O)NC(C)C)c(Cl)c5C4)C3)c12]

### 3-chloro-5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5, 6,7,8-tetrahydropyrido[3,4-d]pyrimidin-4-yl)-N-isopropyl-4,5,6,7,8,9-hexahydropyrazolo[1,5-a][1,4] diazocine-2-carboxamide

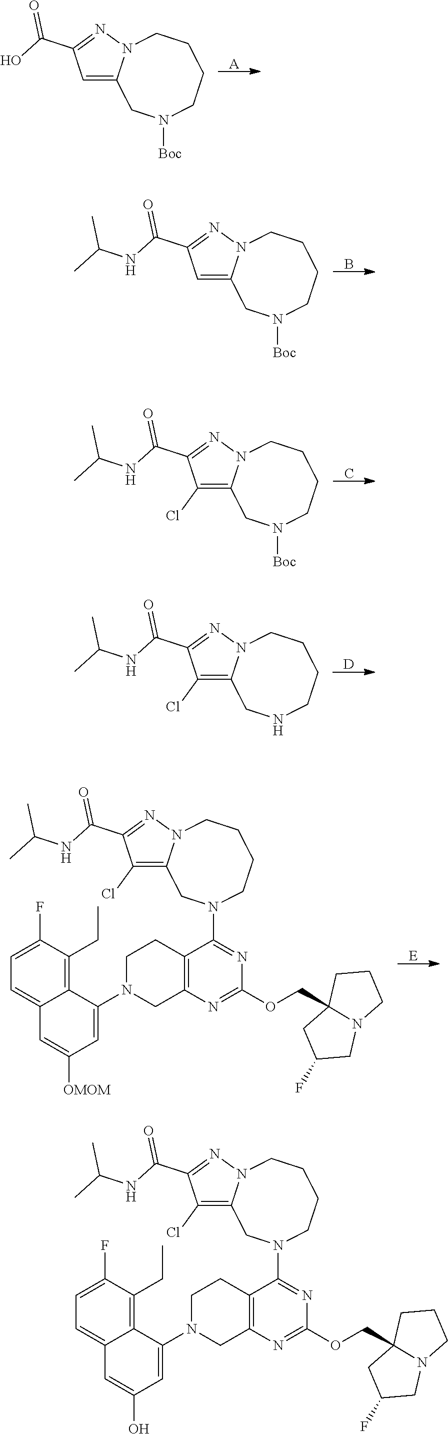

Step A. tert-butyl 2-(isopropylcarbamoyl)-6,7,8,9-tetrahydropyrazolo[1,5-a][1,4] diazocine-5(4H)-carboxylate: To a solution of 5-(tert-butoxycarbonyl)-4,5,6,7,8,9-hexahydropyrazolo[1,5-a][1,4] diazocine-2-carboxylic acid (500 mg, 1.0 equiv) in THF (5.0 mL) were added DIEA (656 mg, 3.0 equiv) and HATU (965 mg, 1.5 equiv). The reaction was stirred at 20° C. for 30 minutes. A solution of propan-2-amine (500 mg 5.0 equiv) in THF (2 mL) was added. The reaction was stirred at 20° C. for 5 hours. The mixture was diluted with water (10 mL), concentrated under reduced pressure and extracted, with EtOAc (3×40 mL). The combined organic layers were washed with brine (40 mL), dried over anhydrous sodium sulfate, concentrated, and purified by prep-TLC (SiO₂, petroleum ether/ethyl acetate=1/1) to afford the title compound (540 mg, 95% yield) as yellow solid; LCMS [ESI, M+1]: m/z=337.3.

Step B. tert-butyl 3-chloro-2-(isopropylcarbamoyl)-6,7,8,9-tetrahydropyrazolo[1,5-a][1,4]diazocine-5(4H)-carboxylate: To a solution of tert-butyl 2-(isopropylcarbamoyl)-6,7,8,9-tetrahydropyrazolo[1,5-a][1,4] diazocine-5(4H)-carboxylate (540 mg, 1.0 equiv) in DMA (0.5 mL) and AcOH (0.05 mL) was added 1-chloropyrrolidine-2,5-dione (39.7 mg, 2.0 equiv). The reaction was stirred at 25° C. for 3 hours. The mixture was poured into water (2 mL) and extracted with ethyl acetate (3×5 mL). The combined organic layers were washed with brine (10 mL), dried over anhydrous sodium sulfate, concentrated and purified by prep-TLC (SiO2, petroleum ether/ethyl acetate=1/1) to afford the title compound (500 mg, 79% yield) as white solid; LCMS (ESI, M+1): m/z=371.2

Step C. 3-chloro-N-isopropyl-4,5,6,7,8,9-hexahydropyrazolo[1,5-a][1,4] diazocine-2-carboxamide: To a solution of tert-butyl 3-chloro-2-(isopropylcarbamoyl)-6,7,8,9-tetrahydro-4H-pyrazolo[1,5-a][1,4] diazocine-5-carboxylate (500 mg, 1.0 equiv) in MeCN (1.0 mL) was added HCl•dioxane (4 M, 1.0 mL, 2.0 equiv). The reaction was stirred at 20° C. for 2 hours. The mixture was concentrated under reduced pressure to afford the title compound (320 mg, crude) as white solid; LCMS [ESI, M+1]: m/z=271.1

The last two steps were performed according to Example 248. The title compound was obtained as off-white solid (FA salt). ¹H NMR (400 MHz, METHANOL-d₄) δ=8.53 (br s, 1H), 7.51 (dd, J=5.8, 9.2 Hz, 1H), 7.15 (t, J=9.6 Hz, 1H), 7.01-6.94 (m, 2H), 5.35-5.20 (m, 1H), 4.47-4.24 (m, 2H), 4.17 (td, J=6.4, 13.2 Hz, 1H), 4.09 (br d, J=17.6 Hz, 1H), 4.01-3.79 (m, 3H), 3.76-3.64 (m, 2H), 3.55-3.48 (m, 1H), 3.45-3.36 (m, 2H), 3.29-3.12 (m, 5H), 3.08-2.95 (m, 1H), 2.73 (br d, J=12.8 Hz, 1H), 2.37-1.69 (m, 11H), 1.29 (br s, 1H), 1.24 (dd, J=2.3, 6.4 Hz, 6H), 1.12 (t, J=7.2 Hz, 3H); LCMS [ESI, M+1]: m/z=749.5.

### Example 507

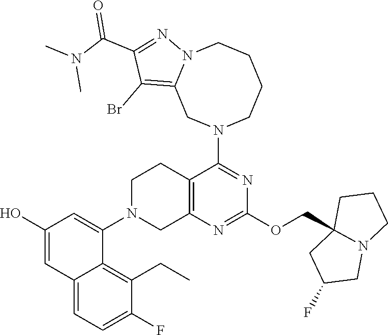 [SMILES: CCc1c(F)ccc2cc(O)cc(N3CCc4c(nc(OC[C@@]56CCCN5C[C@H](F)C6)nc4N4CCCCn5nc(C(=O)N(C)C)c(Br)c5C4)C3)c12]

### 3-bromo-5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-N,N-dimethyl-4,5,6,7,8,9-hexahydropyrazolo[1,5-a][1,4] diazocine-2-carboxamide

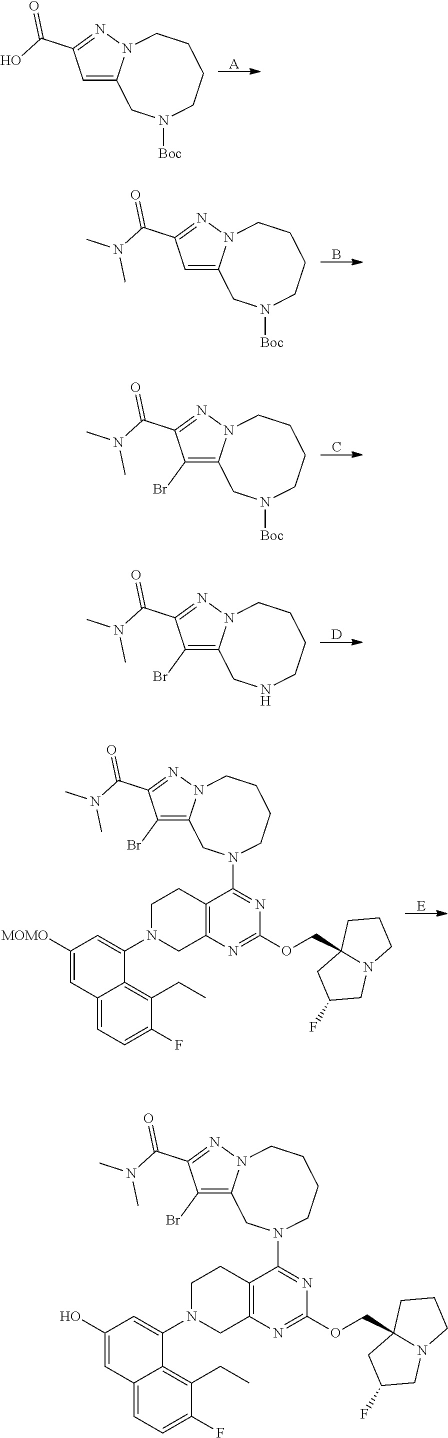

Step A. tert-butyl 2-(dimethylcarbamoyl)-6,7,8,9-tetrahydropyrazolo[1,5-a][1,4] diazocine-5(4H)-carboxylate: To a solution of 5-(tert-butoxycarbonyl)-4,5,6,7,8,9-hexahydropyrazolo[1,5-a][1,4] diazocine-2-carboxylic acid (1.00 g, 1.0 equiv), N-methylmethanamine (552 mg 2.0 equiv, HCl salt) and DIEA (2.19 g, 5.0 equiv) in dichlomethane (10 mL) was added HATU (2.57 g, 2 equiv). The reaction was stirred at 25° C. for 2 hours. The mixture was poured into ice water (10 mL) and extracted with ethyl acetate (3×30 mL). The combined organic layers were washed with brine (50 mL), dried over sodium sulfate, filtered, concentrated, and purified by column chromatography [SiO₂, petroleum ether/ethyl acetate=3/1 to 1/1] to afford the title compound (900 mg, 75% yield) as yellow solid.

Step B. tert-butyl 3-bromo-2-(dimethylcarbamoyl)-6,7,8,9-tetrahydropyrazolo[1,5-a][1,4] diazocine-5(4H)-carboxylate: To a solution of tert-butyl 2-(dimethylcarbamoyl)-6,7,8,9-tetrahydropyrazolo[1,5-a][1,4] diazocine-5(4H)-carboxylate (500 mg, 1.0 equiv) in DMA (2 mL) were added NBS (552 mg, 2.0 equiv) and TFA (0.2 mL, 2.25 equiv) at 25° C. The reaction was stirred at 60° C. for 2 hours. The mixture was poured into ice water (10 mL) and the resulting mixture was extracted with ethyl acetate (3×30 mL). The combined organic layers were filtered, concentrated, and purified by column chromatography, (SiO₂, Petroleum ether/Ethyl acetate=3/1 to 1/1) to afford the title compound (520 mg,74% yield) as yellow solid. ¹H NMR (400 MHz, CHLOROFORM-d) δ=7.31-7.08 (m, 1H), 4.36-4.17 (m, 2H), 3.63 (br s, 2H), 3.47 (br s, 1H), 2.94-2.80 (m, 2H), 1.79 (br s, 2H), 1.42 (br s, 9H), 1.09-1.01 (m, 2H), 1.00-0.92 (m, 2H); LCMS (ESI, M+1): m/z=403.1.

Step C. 3-bromo-N,N-dimethyl-4,5,6,7,8,9-hexahydropyrazolo[1,5-a][1,4] diazocine-2-carboxamide: To a solution of tert-butyl 3-bromo-2-(dimethylcarbamoyl)-6,7,8,9-tetrahydropyrazolo[1,5-a][1,4] diazocine-5(4H)-carboxylate (330 mg, 1.0 equiv) in MeOH (1 mL) was added HCl-MeOH (4 M, 5 mL, 24.3 equiv). The mixture was stirred at 25° C. for 1 hour. The reaction was concentrated to afford the title compound (240 mg, crude, HCl salt) as yellow oil.

The last two steps were performed according to Example 248. The title compound was obtained as light-yellow solid. ¹H NMR (400 MHz, METHANOL-d₄) δ=1.05-1.19 (m, 3H) 1.82-2.35 (m, 10H) 2.61-2.77 (m, 1H) 2.98-3.07 (m, 1H) 3.10 (s, 6H) 3.15-3.28 (m, 4H) 3.35-3.53 (m, 3H) 3.58-3.79 (m, 2H) 3.86-4.18 (m, 4H) 4.34-4.48 (m, 2H) 4.55-4.74 (m, 1H) 4.82 (br d, J=6.36 Hz, 1H) 4.91-4.98 (m, 1H) 5.18-5.40 (m, 1H) 6.94-7.01 (m, 2H) 7.15 (t, J=9.41 Hz, 1H) 7.48-7.55 (m, 1H); LCMS (ESI, M+1): m/z=779.5.

### Example 508

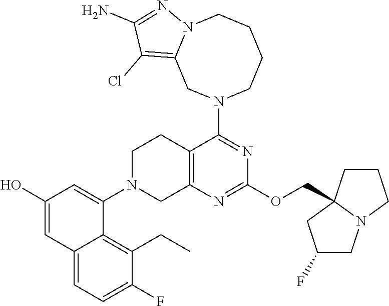 [SMILES: CCc1c(F)ccc2cc(O)cc(N3CCc4c(nc(OC[C@@]56CCCN5C[C@H](F)C6)nc4N4CCCCn5nc(N)c(Cl)c5C4)C3)c12]

### 4-(4-(2-amino-3-chloro-6,7,8,9-tetrahydropyrazolo[1,5-a][1,4] diazocin-5(4H)-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-ol

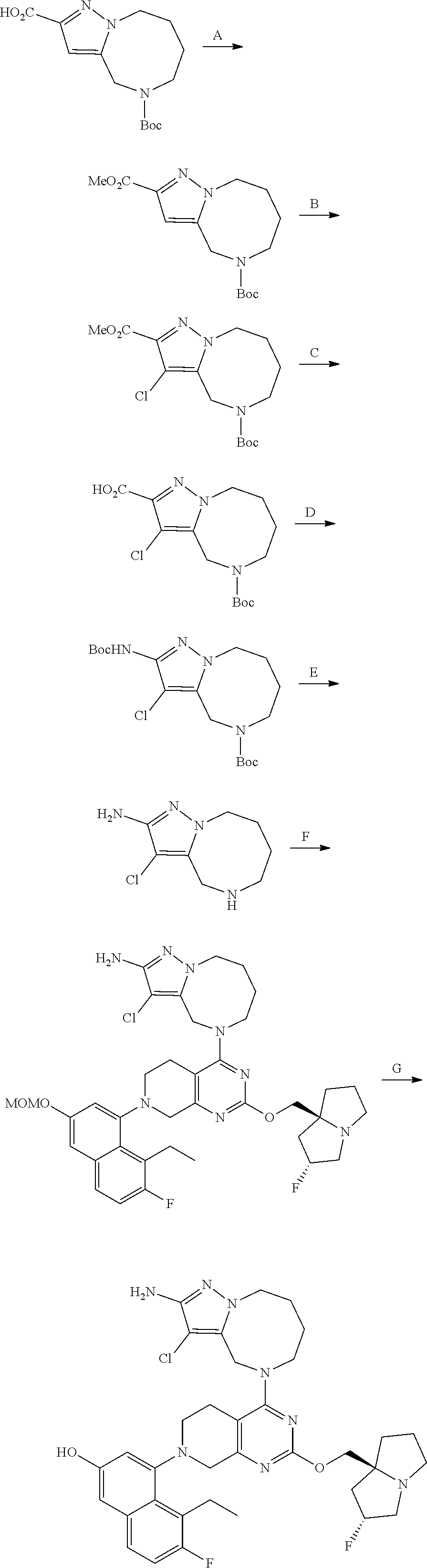

Step A. 5-(tert-butyl) 2-methyl 6,7,8,9-tetrahy dropyrazolo[1,5-a][1,4]diazocine-2,5(4H)-dicarboxylate: To a solution of 5-(tert-butoxycarbonyl)-4,5,6,7,8,9-hexahydropyrazolo[1,5-a][1,4] diazocine-2-carboxylic acid (500 mg, 1.0 equiv) in DCM (5 mL) and MeOH (2.5 mL) was added diazomethyl(trimethyl)silane (2 M, 4.23 mL, 5.0 equiv). The reaction was stirred at 25° C. for 0.5 hours. The mixture was concentrated, dissolved in DCM (5 mL) and washed with saturated NaHCO₃ solution (10 ml×2). The organic phase was washed with brine (10 mL), dried over anhydrous sodium sulfate, and concentrated to afford the title compound (380 mg, 72% yield) as yellow oil. ¹H NMR (400 MHz, DMSO-d₆) δ=6.62 (br s, 1H), 4.54 (s, 2H), 4.41 (br s, 2H), 3.77 (s, 3H), 3.35 (br s, 2H), 1.81 (br s, 2H), 1.39 (br d, J=5.2 Hz, 11H); L CMS (ESI, M+1): m/z=310.0.

Step B to G were performed according to Example 358. The title compound was obtained as as off-white solid (FA salt). ¹H NMR (400 MHz, DMSO-d₆) δ=9.85-9.57 (m, 1H), 8.18 (br s, 1H), 7.59 (dd, J=6.0, 9.2 Hz, IH), 7.24 (t, J=9.6 Hz, 1H), 6.98 (s, 2H), 5.33-5.13 (m, 1H), 4.83-4.71 (m, 3H), 4.69-4.60 (m, 1H), 4.30-4.17 (m, 1H), 4.02 (br d, J=14.0 Hz, 1H), 3.97-3.86 (m, 2H), 3.84-3.78 (m, 1H), 3.67-3.55 (m, 2H), 3.51-3.39 (m, 3H), 3.16-3.08 (m, 2H), 3.07-3.00 (m, 2H), 2.96 (br s, 1H), 2.83-2.75 (m, 1H), 2.52 (br s, 2H), 2.07-2.02 (m, 1H), 1.99-1.94 (m, 1H), 1.92 (br s, 1H), 1.84-1.76 (m, 2H), 1.75-1.62 (m, 4H), 1.61-1.47 (m, 1H), 1.05 (t, J=7.2 Hz, 3H). LCMS (ESI, M+1). m/z=679.4.

### Example 509

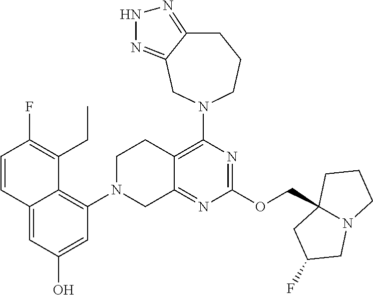 [SMILES: CCc1c(F)ccc2cc(O)cc(N3CCc4c(nc(OC[C@@]56CCCN5C[C@H](F)C6)nc4N4CCCc5n[nH]nc5C4)C3)c12]

### 4-(4-(7, 8-dihydro-[1,2,3]triazolo[4, 5-c]azepin-5 (2H,4H, 6H)-yl)-2-(((2R, 7aS)-2-fluorohexahydro-1H-pyrrolizin-7a-yl)methoxy)-5,6-dihydropyrido[3,4-d]pyrimidin-7(8H)-yl)-5-ethyl-6-fluoronaphthalen-2-ol

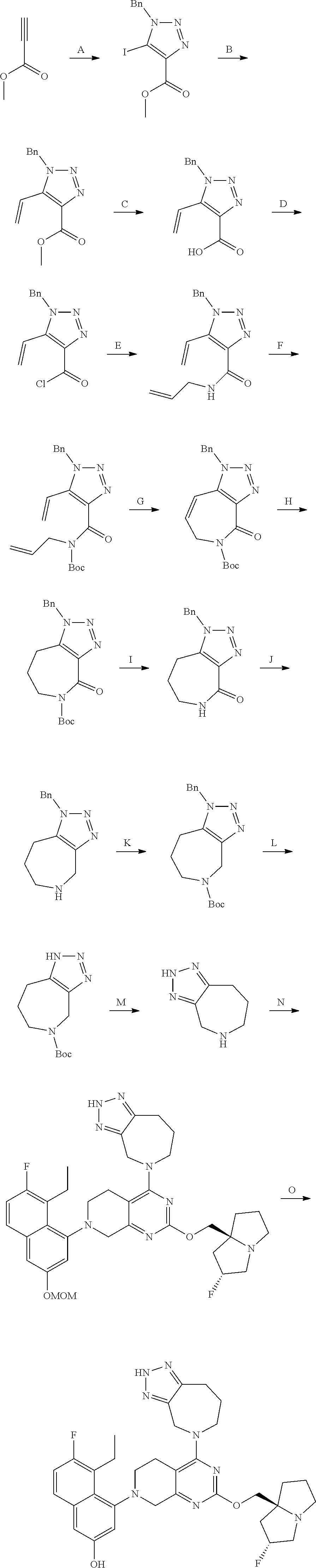

Step A. methyl 1-benzyl-5-iodo-1H-1,2,3-triazole-4-carboxylate: To a mixture of methyl propiolate (12.5 g, 1.0 equiv), CuI (31.1 g, 1.1 equiv), NBS (31.8 g, 1.2 equiv) and DIEA (19.2 g, 1.2 equiv) in tetrahydrofuran (2.5 L) was added azidomethylbenzene (21.0 g, 1.1 eq). The reaction was stirred at 25° C. for 6 hours. The mixture was diluted with water (200 mL) and extracted with ethyl acetate (3×200 mL). The organic layers were washed with brine (100 mL), dried over Na₂SO₄, concentrated, and purified with column chromatography (SiO₂, Petroleum ether/Ethyl acetate=10/1 to 3/1) to afford the title compound (30.0 g, 59% yield) as yellow solid. ¹H NMR (400 MHz, CHLOROFORM-d) δ=7.38-7.32 (m, 3H), 7.30-7.25 (m, 2H), 5.67 (s, 2H), 3.97 (s, 3H).

Step B. methyl 1-benzyl-5-vinyl-1H-1,2,3-triazole-4-carboxylate: To a mixture of methyl 1-benzyl-5-iodo-1H-1,2,3-triazole-4-carboxylate (8.00 g, 1.0 equiv), 4,4,5,5-tetramethyl-2-vinyl-1,3,2-dioxaborolane (7.18 g, 2.0 equiv) and Na₂CO₃ (7.41 g, 3.0 equiv) in water (16 mL) and dioxane (80 mL) was added Pd(dppf)Cl₂ (1.71 g, 0.1 equiv). The reaction was stirred at 60° C. for 2 hours under N₂ atmosphere. The mixture was diluted with water (50 mL) and extracted with ethyl acetate (3×50 mL). The combined organic layers were washed with brine (50 mL), dried over Na₂SO₄, concentrated, and purified with column chromatography (SiO₂, Petroleum ether/Ethyl acetate=10/1 to 5/1) to afford the title compound (3.90 g, 69% yield) as yellow solid. ¹H NMR (400 MHz, CHLOROFORM-d) δ=7.38-7.30 (m, 3H), 7.17-7.11 (m, 2H), 6.84 (dd, J=12.0, 18.0 Hz, 1H), 6.03-5.96 (m, 1H), 5.76 (d, J=12.0 Hz, 1H), 5.65 (s, 2H), 3.96 (s, 3H).

Step C. 1-benzyl-5-vinyl-1H-1,2,3-triazole-4-carboxylic acid. To a solution of methyl 1-benzyl-5-vinyl-1H-1,2,3-triazole-4-carboxylate (3.90 g, 1.0 equiv) in tetrahydrofuran (20 mL) and H₂O (20 mL) was added NaOH (6.41 g, 10 equiv). The reaction was stirred at 25° C. for 2 hours. The mixture was acidified with 1 M HCl aqueous solution to pH=1, filtered and concentrated to afford the title compound (3.5 g, 95% yield) as white solid. ¹H NMR (400 MHz, DMSO-d₆) 5=7.40-7.28 (m, 3H), 7.14 (br d, J=7.2 Hz, 2H), 6.92 (dd, J=12.0, 18.0 Hz, 1H), 6.08 (d, J=18.0 Hz, 1H), 5.80-5.73 (m, 3H).

Step D. 1-benzyl-5-vinyl-1H-1,2,3-triazole-4-carbonyl chloride: To a solution of 1-benzyl-5-vinyl-1H-1,2,3-triazole-4-carboxylic acid (3.5 g, 1.0 equiv) and dimethyl formamide (111 mg, 0.1 equiv) in dichloromethane (40 mL) was added oxalyl chloride (2.81 g, 1.5 equiv) at 0° C. The reaction was stirred at 25° C. for 0.5 hour. The mixture was concentrated to afford the title compound (3.78 g, 99% yield) as white solid.

Step E. N-allyl-1-benzyl-5-vinyl-1H-1,2,3-triazole-4-carboxamide: To a solution of prop-2-en-1-amine (2.15 g, 1.5 equiv, HCl salt) and trimethylamine (6.21 g, 4.0 equiv) in tetrahydrofuran (40 mL) was added 1-benzyl-5-vinyl-1H-1,2,3-triazole-4-carbonyl chloride (3.78 g, 1.0 equiv) at 0° C. The reaction was stirred at 25° C. for 1 hour. The mixture was diluted with water (20 mL) and extracted with ethyl acetate (3×20 mL). The combined organic layers were washed with brine (20 mL), dried over Na₂SO₄ and concentrated under reduced pressure to afford the title compound (3.50 g, 84% yield) as white solid; ¹H NMR (400 MHz, CHLOROFORM-d) 6=7.51 (br s, 1H), 7.37-7.25 (m, 3H), 7.16-7.08 (m, 2H), 6.98-6.86 (m, 1H), 6.20-6.10 (m, 1H), 5.98-5.86 (m, 1H), 5.73-5.66 (m, 1H), 5.65-5.59 (m, 2H), 5.32-5.22 (m, 1H), 5.20-5.11 (m, 1H), 4.07 (tt, J=1.6, 6.0 Hz, 2H); LCMS (ESI, M+1): m/z=269.1.

Step F. tert-butyl allyl(1-benzyl-5-vinyl-1H-1,2,3-triazole-4-carbonyl)carbamate: To a solution of N-allyl-1-benzyl-5-vinyl-1H-1,2,3-triazole-4-carboxamide (50.0 mg, 1.0 equiv) and (Boc)20 (81.3 mg, 2.0 equiv) in dichloromethane (2 mL) was added DMAP (22.8 mg, 1.0 equiv). The reaction was stirred at 40° C. for 6 hours. The mixture was diluted with water (1 mL) and extracted with ethyl acetate (3×1 mL). The combined organic layers were washed with brine (1 mL), dried over Na₂SO₄, concentrated and purified by reversed phase chromatography column [water (FA, 0.1%)/acetonitrile] to afford the title compound (34.0 mg, 50% yield) as white solid. ¹H NMR (400 MHz, CHLOROFORM-d) δ=7.38-7.31 (m, 3H), 7.22 (dd, J=1.6, 7.6 Hz, 2H), 6.70-6.59 (m, 1H), 6.02-5.90 (m, 2H), 5.69-5.63 (m, 1H), 5.61-5.56 (m, 2H), 5.37-5.28 (m, 1H), 5.22-5.16 (m, 1H), 4.45-4.38 (m, 2H), 1.28 (s, 9H).

Step G. tert-butyl 1-benzyl-4-oxo-4,6-dihydro-[1,2,3]triazolo[4,5-c]azepine-5(1H)-carboxylate: To a mixture of tert-butyl allyl(1-benzyl-5-vinyl-1H-1,2,3-triazole-4-carbonyl)carbamate (280 mg, 1.0 equiv) in dichloromethane (5 mL) was added (1,3-Bis(2,4,6-trimethylphenyl)-2-imidazolidinylidene)dichloro(phenylmethylene)(tricyclohexylphosphine)ruthenium (64.4 mg, 0.1 equiv). The reaction was stirred at 40° C. for 12 hours under N₂ atmosphere. The mixture was diluted with water (5 mL) and extracted with dichloromethane (3×5 mL). The combined organic layers were washed with brine (5 mL), dried over Na₂SO₄, concentrated and purified by reversed phase chromatography [water (FA, 0.1%)/acetonitrile] to afford the title compound (34.0 mg, 50% yield) as white solid; ¹H NMR (400 MHz, CHLOROFORM-d) 5=7.40-7.33 (m, 3H), 7.21 (dd, J=2.0, 7.2 Hz, 2H), 6.70-6.66 (m, 1H), 6.63-6.56 (m, 1H), 5.62 (s, 2H), 4.20 (d, J=6.5 Hz, 2H), 1.51 (s, 9H); LCMS (ESI, M-99): m/z=241.1.

Step H. tert-butyl 1-benzyl-4-oxo-4,6,7,8-tetrahydro-[1,2,3]triazolo[4,5-c]azepine-5(1H)-carboxylate: To a suspension of Pd/C (650 mg, 10% purity) in tetrahydrofuran (10 mL) was added tert-butyl 1-benzyl-4-oxo-4,6-dihydro-[1,2,3]triazolo[4,5-c]azepine-5(1H)-carboxylate (650 mg, 1.0 equiv) under N₂ atmosphere. The reaction was stirred at 25° C. for 2 hours under H₂ atmosphere (15 psi). The mixture was filtered and the filter cake was washed with EtOAc (3×10 mL). The filtrate was concentrated under reduced pressure to afford the title compound (550 mg, 84% yield) as yellow solid; ¹H NMR (400 MHz, CILOROFORM-d) 5=7.30-7.23 (m, 3H), 7.11-7.05 (m, 2H), 5.50-5.46 (m, 2H), 3.72-3.63 (m, 2H), 2.76-2.68 (m, 2H), 2.01-1.92 (m, 2H), 1.45 (s, 9H).

Step I. 1-benzyl-5,6,7,8-tetrahydro-[1,2,3]triazolo[4,5-c]azepin-4(1H)-one: To a solution of tert-butyl 1-benzyl-4-oxo-4,6,7,8-tetrahydro-[1,2,3]triazolo[4,5-c]azepine-5(1H)-carboxylate (50.0 mg, 1.0 equiv) in methanol (2 mL) was added HCl (4 M in methanol, 2 mL, 55 equiv) at 0° C. The reaction was stirred at 25° C. for 1 hour. The reaction was concentrated to afford the title compound (20.0 mg, 57% yield) as white solid.

Step J. 1-benzyl-1,4,5,6,7,8-hexahydro-[1,2,3]triazolo[4,5-c]azepine: To a solution of 1-benzyl-5,6,7,8-tetrahydro-[1,2,3]triazolo[4,5-c]azepin-4(1H)-one (600 mg, 1.0 equiv) in tetrahydrofuran (20 mL) was added BH3-Me2S (10 M, 743 μL, 3.0 equiv) at 0° C. The reaction was stirred at 65° C. for 12 hrous under N₂ atmosphere. The mixture was quenched with methanol 5 mL at 0° C. under N₂ and concentrated under reduce pressure to remove the solvent. The residue was diluted with water (5 mL) and extracted with ethyl acetate (3×5 mL). The combined organic layers were washed with brine (5 mL), dried over Na₂SO₄ and concentrated to afford the title compound (600 mg, 99% yield) as yellow solid.

Step K. tert-butyl 1-benzyl-4,6,7,8-tetrahydro-[1,2,3]triazolo[4,5-c]azepine-5(1H)-carboxylate: To a solution of 1-benzyl-1,4,5,6,7,8-hexahydro-[1,2,3]triazolo[4,5-c]azepine (600 mg, 1.0 equiv) and triethylamine (2.66 g, 10 equiv) in methanol (10 mL) was added (Boc)20 (2.29 g, 4.0 equiv). The reaction was stirred at 40° C. for 12 hours. The mixture was concentrated, dissolved in water (10 ml) and extracted with ethyl acetate (3×10 mL). The combined organic layers were washed with brine (10 mL), dried over Na₂SO₄, concentrated, and purified with prep-HPLC (Unisil 3-100 C18 Ultra 150×50 mm×3 μm; A: water (10 mM HCOOH); B:ACN, B: 38%-68% over 7 min) to afford the title compound (40.0 mg, 4.6% yield) as white solid. ¹H NMR (400 MHz, CHLOROFORM-d) δ=7.35-7.28 (m, 3H), 7.15-7.08 (m, 2H), 5.49-5.45 (m, 2H), 4.75-4.58 (m, 2H), 3.65-3.49 (m, 2H), 2.65-2.56 (m, 2H), 1.89-1.76 (m, 2H), 1.48-1.36 (m, 9H); LCMS (ESI, M+1): m/z=329.1.

Step L. tert-butyl 4,6,7,8-tetrahydro-[1,2,3]triazolo[4,5-c]azepine-5(1H)-carboxylate: To a suspension of Pd/C (20.0 mg, 10% purity) in ethanol (2 mL) were added tert-butyl 1-benzyl-4,6,7,8-tetrahydro-[1,2,3]triazolo[4,5-c]azepine-5(1H)-carboxylate (30.0 mg, 1.0 equiv) and AcOH (548 ug, 0.1 eq) under N₂ atmosphere. The reaction was stirred at 80° C. for 8 hours under H₂ atmosphere (15 psi). The mixture was filtered through celite. The filter cake was washed with ethyl acetate (3×1 mL), and the filtrate was concentrated to afford the title compound (20.0 mg, 87% yield) as white solid; LCMS (ESI, M+1): m/z=239.1.

Step M. 2,4,5,6,7,8-hexahydro-[1,2,3]triazolo[4,5-c]azepine hydrochloride: To a solution of tert-butyl 4,6,7,8-tetrahydro-[1,2,3]triazolo[4,5-c]azepine-5(1H)-carboxylate (100 mg, 1.0 equiv) in methanol (5 mL) was added HCl (4 M in methanol, 5 mL, 66 equiv) at 0° C. The reaction was stirred at 0° C. for 1 hour. The mixture was concentrated under reduced pressure to afford the title compound (70.0 mg, 96% yield, HCl) as white solid; ¹H NMR (400 MHz, DMSO-d₆) δ=9.96-9.73 (m, 2H), 4.35-4.21 (m, 2H), 3.43-3.37 (m, 2H), 2.95-2.83 (m, 2H), 2.03-1.92 (m, 2H).

The last two steps were performed according to Example 248. The title compound was obtained as white solid. ¹H NMR (400 MHz, METHANOL-d₄) δ=7.50 (dd, J=6.0, 8.8 Hz, 1H), 7.16-7.10 (m, 1H), 6.98-6.93 (m, 2H), 5.35-5.17 (m, 1H), 5.05 (dd, J=6.8, 15.6 Hz, 1H), 4.82-4.74 (m, 2H), 4.30-4.20 (m, 1H), 4.11-4.02 (m, 2H), 4.02-3.92 (m, 2H), 3.70-3.60 (m, 1H), 3.54-3.45 (m, 1H), 3.40-3.33 (m, 2H), 3.24-3.15 (m, 4H), 3.04-2.98 (m, 1H), 2.98-2.92 (m, 2H), 2.76-2.64 (m, 1H), 2.29-2.04 (m, 4H), 1.99-1.78 (m, 4H), 1.12-1.06 (m, 3H); LCMS (ESI, M+1): m/z=617.3.

### Example 510

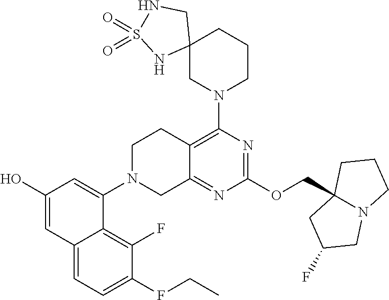

### 7-(7-(7-(ethylthio)-8-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-2-thia-1,3,7-triazaspiro[4.5]decane 2,2-dioxide

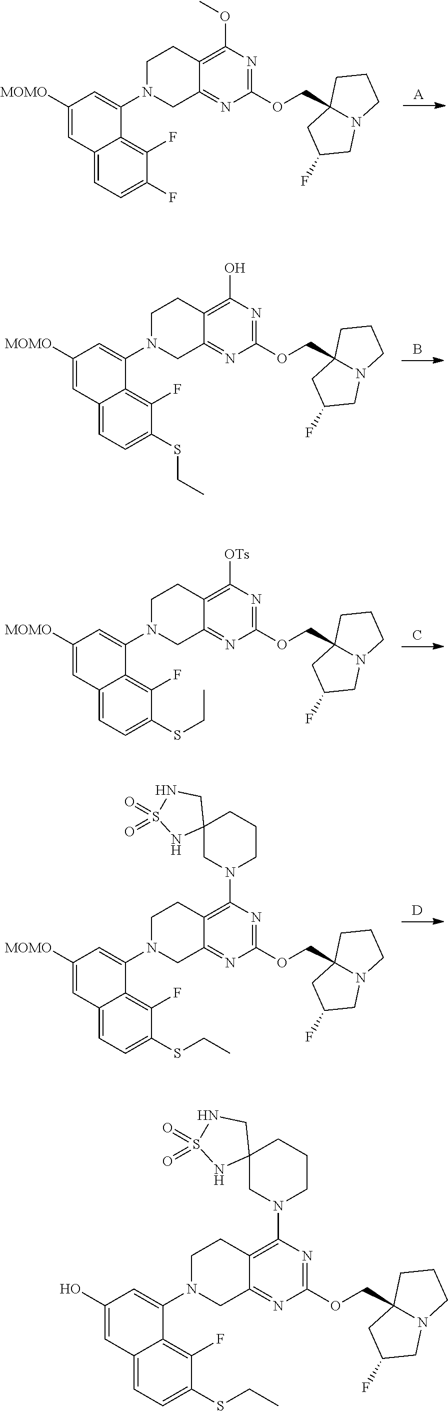

Step A. 7-(7-(ethylthio)-8-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-ol: To a solution of EtSH (1.03 g, 32 equiv) in DMAC (4 mL) was added NaH (164 mg, 60% purity, 8.0 equiv) at 0′° C. The mixture was stirred at 0° C. for 0.5 hour. 7-(7,8-difluoro-3-(methoxymethoxy)naphthalen-1-yl)-2-(((2R,7aS)-2-fluorohexahydro-1H-pyrrolizin-7a-yl)methoxy)-4-methoxy-5,6,7,8-tetrahydropyrido[3,4-d]pyri midine (280 mg, 1.0 equiv) was added. The reaction was stirred at 60° C. for another 1 hour. The mixture was diluted with water (4 mL) and the pH was adjusted to 6 with 2 M HCl aqueous solution. The mixture was extracted with EtOAc (3×4 mL). The combined organic layers were washed with brine (2×4 mL), dried over anhydrous sodium sulfate, concentrated, and purified with reversed phase chromatography HPLC (0.1% FA condition) to afford the title compound (310 mg, 89% yield) as yellow solid. LCMS (ESI, M+1):m/z=573.2.

Step B. 7-(7-(ethylthio)-8-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl 4-methylbenzenesulfonate: To a mixture of 7-(7-(ethylthio)-8-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-ol (300 mg, 1.0 equiv), DIEA (203 mg, 3.0 equiv) and DMAP (6.40 mg, 0.1 equiv) in DCM (3 mL) was added TsC1 (150 mg, 1.5 equiv) at 0° C. The reaction was stirred at 25° C. for 0.5 hour. The mixture was concentrated and purified with HPLC (0.1% FA condition) to afford the title compound (160 mg, 42% yield) as yellow solid. LCMS (ESI, M+1): m/z=727.4.

The last two steps were performed according to Example 248. The title compound was obtained as orange solid (FA salt). ¹H NMR (400 MHz, METHANOL-d₄) δ=7.46-7.34 (m, 2H), 6.81 (br d, J=17.6 Hz, 2H), 5.58-5.35 (m, 1H), 4.45-4.31 (m, 2H), 4.28-4.16 (m, 1H), 4.10-3.70 (m, 4H), 3.68-3.57 (m, 4H), 3.55-3.43 (m, 2H), 3.21 (d, J=11.6 Hz, 1H), 2.98-2.80 (m, 3H), 2.79-2.51 (m, 2H), 2.50-2.25 (m, 3H), 2.23-2.15 (m, 2H), 2.14-1.90 (m, 3H), 1.83 (br d, J=9.2 Hz, 3H), 1.25 (t, J=7.2 Hz, 3H); LCMS (ESI, M+1): m/z=702.7.

### Example 511

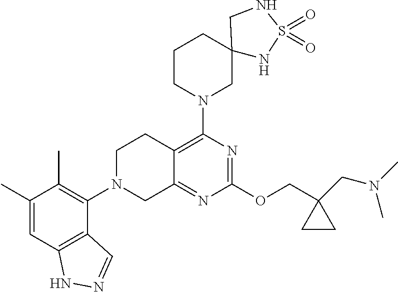

### 7-(7-(5,6-dimethyl-1H-indazol-4-yl)-2-((1-((dimethylamino)methyl)cyclopropyl)methoxy)-5,6,7,8-tetrahydropyrido[3,4-d]pyrimidin-4-yl)-2-thia-1,3,7-triazaspiro[4.5]decane 2,2-dioxide

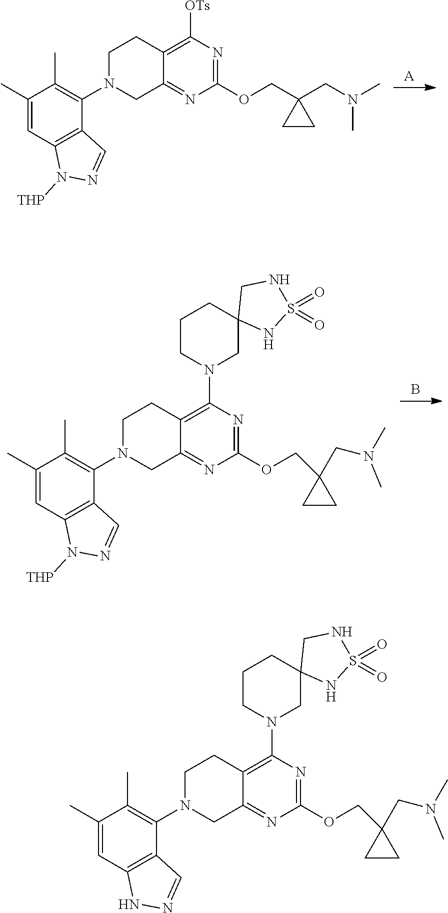

Synthesized according to Example 462. The title compound was obtained as yellow solid (FA salt). ¹H NMR (400 MHz, METHANOL-d₄) δ=8.04 (s, 1H), 7.20 (s, 1H), 4.60 (br s, 1H), 4.28 (s, 2H), 4.24-4.12 (m, 2H), 4.04-3.92 (m, 1H), 3.82-3.68 (m, 1H), 3.56-3.46 (m, 3H), 3.44-3.36 (m, 2H), 3.20 (br d, J=11.6 Hz, 1H), 3.12-3.00 (m, 2H), 2.82 (br s, 7H), 2.40 (s, 3H), 2.34 (s, 3H), 2.04-1.92 (m, 1H), 1.88-1.68 (m, 3H), 0.96-0.68 (m, 4H); LCMS (ESI, M+1): m/z=596.1.

### Example 512

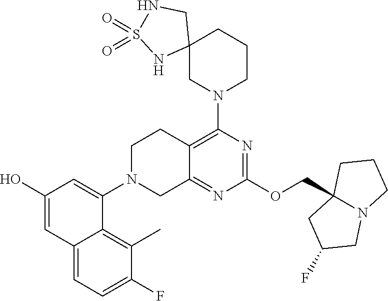 [SMILES: Cc1c(F)ccc2cc(O)cc(N3CCc4c(nc(OC[C@@]56CCCN5C[C@H](F)C6)nc4N4CCCC5(CNS(=O)(=O)N5)C4)C3)c12]

### 7-(7-(7-fluoro-3-hydroxy-8-methylnaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-2-thia-1,3,7-triazaspiro[4.5]decane 2,2-dioxide

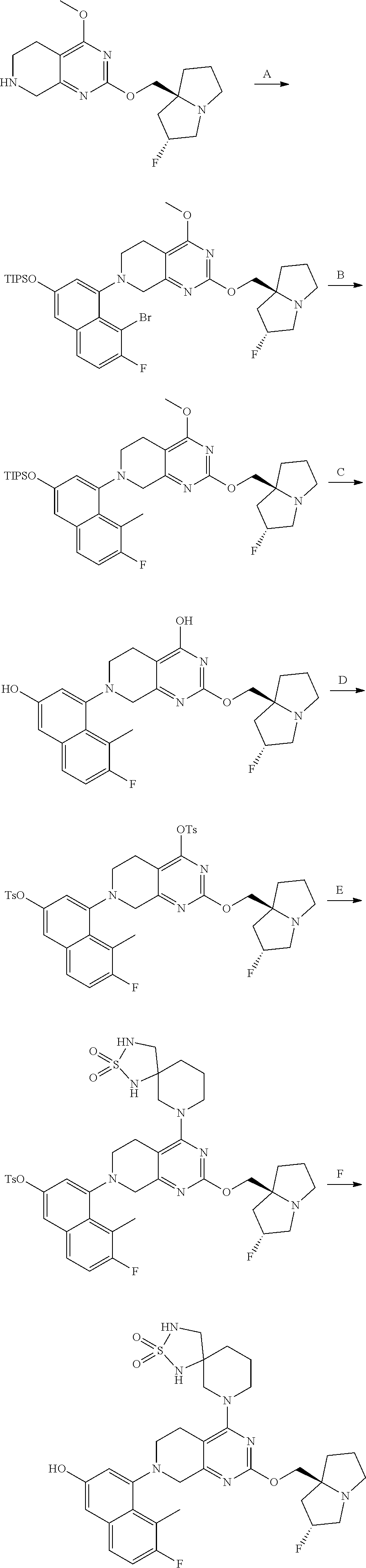

Step A. 7-(8-bromo-7-fluoro-3-((triisopropylsilyl)oxy)naphthalen-1-yl)-2-(((2R,7aS)-2-fluorohexahydro-11H-pyrrolizin-7a-yl)methoxy)-4-methoxy-5,6,7,8-tetrahydropyrido[3,4-d]pyrimidine: To a mixture of 2-(((2R,7aS)-2-fluorohexahydro-1H-pyrrolizin-7a-yl)methoxy)-4-methoxy-5,6,7,8-tetrahydropyrido[3,4-d]pyrimidine (800 mg, 1 equiv), Cs₂CO₃ (2.43 g, 3.0 equiv) and 8-bromo-7-fluoro-3-((triisopropylsilyl)oxy)naphthalen-1-yl trifluoromethanesulfonate (2.03 g, 1.5 equiv) in toluene (8 mL) were added tris(dibenzylideneacetone)dipalladium(0) (227 mg, 0.1 equiv) and BINAP (309 mg, 0.2 equiv). The reaction was stirred at 70° C. for 12 hours under N₂. The mixture was filtered, concentrated, and purified by HPLC (0.1% FA condition) to afford the title compound (330 mg, 17% yield) as brown solid. LCMS (ESI, M+1, M+3): m/z=717.4, 719.4.

Step B. 7-(7-fluoro-8-methyl-3-((triisopropylsilyl)oxy)naphthalen-1-yl)-2-(((2R,7aS)-2-fluorohexahydro-1H-pyrrolizin-7a-yl)methoxy)-4-methoxy-5,6,7,8-tetrahydropyrido[3,4-d] pyrimidine: To a mixture of 7-(8-bromo-7-fluoro-3-((triisopropylsilyl)oxy)naphthalen-1-yl)-2-(((2R,7aS)-2-fluorohexahydro-1H-pyrrolizin-7a-yl)methoxy)-4-methoxy-5,6,7,8-tetrahydropyrido[3,4-d]pyrimidine (190 mg, 1.0 equiv), methylboronic acid (317 mg, 20 equiv) and K₃PO₄ (169 mg, 3.0 equiv) in H₂O (0.6 mL) and methoxycyclopentane (2 mL) was added CataCXium A Pd G3 (19.3 mg, 0.1 equiv). The reaction was stirred at 90° C. for 2 hours under N₂. The mixture was diluted with H₂O (2 mL) and extracted with EtOAc (3×3 mL). The combined organic layers were washed with brine (2×3 mL), dried over anhydrous sodium sulfate, concentrated, and purified by reversed phase chromatography HPLC (0.1% FA condition) to afford the title compound (105 mg, 60% yield) as brown solid. LCMS (ESI, M+1): m/z=653.7.

Step C. 7-(7-fluoro-3-hydroxy-8-methylnaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-ol: To a solution of EtSH (38.1 mg, 2.5 equiv) in DMAC (1 mL) was added NaH (24.5 mg, 60% purity, 2.5 equiv) at 0° C. After stirred at 20° C. for 0.5 hour, 7-(7-fluoro-8-methyl-3-((triisopropylsilyl)oxy)naphthalen-1-yl)-2-(((2R,7aS)-2-fluorohexahydro-1H-pyrrolizin-7a-yl)methoxy)-4-methoxy-5,6,7,8-tetrahydropyrido[3,4-d]pyrimidine (160 mg, 1.0 equiv) in DMAC (1 mL) was added into the mixture. The reaction was stirred at 60° C. for 1 hour. The mixture was quenched with ice water (2 mL) and purified by reversed phase chromatography HPLC (0.1% FA condition) to afford the title compound (100 mg, 76% yield) as yellow solid. LCMS (ESI, M+1): m/z=483.4.

Step D. 6-fluoro-4-(2-(((2R,7aS)-2-fluorohexahydro-1H-pyrrolizin-7a-yl)methoxy)-4-(tosvloxy)-5,6-dihydropyrido[3,4-d]pyrimidin-7(8H)-yl)-5-methylnaphthalen-2-vl 4-methylbenzenesulfonate: To a mixture of 7-(7-fluoro-3-hydroxy-8-methylnaphthalen-1-yl)-2-(((2R,7aS)-2-fluorohexahydro-11H-pyrrolizin-7a-yl)methoxy)-5,6,7,8-tetrahydropyrido[3,4-d]pyrimidin-4-ol (100 mg, 207.24 μmol, 1 equiv), DIEA (214 mg, 8.0 equiv) and DMAP (2.53 mg, 0.1 equiv) in DCM (1 mL) was added TsCl (158 mg, 4.0 equiv) at 0° C. The reaction was stirred at 0° C. for 0.25 hr. The mixture was diluted with water (3 mL) and extracted with DCM (3 mL). The combined organic layers were washed with brine (2 ml), dried over anhydrous sodium sulfate, concentrated and purified by reversed phase chromatography HPLC (0.1% FA condition) to afford the title compound (60.0 mg, 33% yield) as brown solid. LCMS (ESI, M+1): m/z=791.3.

Step E. 4-(4-(2,2-dioxido-2-thia-1,3,7-triazaspiro[4.5]decan-7-yl)-2-(((2R,7aS)-2-fluorohexahydro-1H-pyrrolizin-7a-yl)methoxy)-5,6-dihydropyrido[3,4-d]pyrimidin-7(8H)-yl)-6-fluoro-5-methylnaphthalen-2-yl 4-methylbenzenesulfonate: To a mixture of 6-fluoro-4-(2-(((2R,7aS)-2-fluorohexahydro-1H-pyrrolizin-7a-yl)methoxy)-4-(tosyloxy)-5,6-dihydropyrido[3,4-d]pyrimidin-7(8H)-yl)-5-methylnaphthalen-2-yl 4-methylbenzenesulfonate (60.0 mg, 1.0 equiv) and 2-thia-1,3,7-triazaspiro[4.5]decane 2,2-dioxide (21.8 mg, 1.5 equiv) in DMF (0.5 mL) were added DIEA (371 mg, 38 equiv) and 4 Å molecular sieve (10.0 mg). The reaction was stirred at 40° C. for 12 hours. The mixture was filtered and purified by reversed phase chromatography HPLC (0.1% FA condition) to afford the title compound (55.0 mg, 90% yield) as white solid. LCMS (ESI, M+1): m/z=810.6.

Step F. 7-(7-(7-fluoro-3-hydroxy-8-methylnaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-2-thia-1,3,7-triazaspiro[4.5]decane 2,2-dioxide: To a solution of 4-(4-(2,2-dioxido-2-thia-1,3,7-triazaspiro[4.5]decan-7-yl)-2-(((2R,7aS)-2-fluorohexahydro-1H-pyrrolizin-7a-yl)methoxy)-5,6-dihydropyrido[3,4-d]pyrimidin-7(8H)-yl)-6-fluoro-5-methylnaphthalen-2-yl 4-methylbenzenesulfonate (65.0 mg, 1.0 equiv) in MeOH (1 mL) was added NaOH (32.1 mg, 10 equiv). The reaction was stirred at 20° C. for 0.5 hour. The mixture was diluted with H₂O (2 mL) and concentrated under reduced pressure to remove solvent. The residue extracted with DCM (3×3 mL). The combined organic layers were washed with brine (2×3 mL), dried over anhydrous sodium sulfate, concentrated, and purified by prep-HPLC [column: Welch Xtimate C18 150×25 mm×5 μm; mobile phase: [water(NH₄HCO₃)-ACN]; B %: 35%-65%, 2 min] and prep-HPLC [column: Welch Xtimate C18 150×25 mm×5 Im; mobile phase: [water(NH₃H₂O)-ACN]; B %: 50%-80%, 8 min] to afford the title compound (4.10 mg, 7.39% yield) as brown solid. ¹H NMR (400 MHz, METHANOL-d₄) δ=7.49 (td, J=4.4, 9.2 Hz, 1H), 7.13 (dt, J=3.2, 9.2 Hz, 1H), 6.95-6.84 (m, 2H), 5.38-5.16 (m, 1H), 4.18-4.06 (m, 3H), 4.05-3.80 (m, 2H), 3.70-3.62 (m, 1H), 3.61-3.46 (m, 3H), 3.45-3.36 (m, 1H), 3.19 (br d, J=7.2 Hz, 2H), 3.18-3.13 (m, 2H), 3.12-2.95 (m, 2H), 2.78 (br d, J=3.6 Hz, 3H), 2.74-2.59 (m, 1H), 2.36-2.03 (m, 4H), 2.01-1.89 (m, 4H), 1.87-1.71 (m, 3H); LCMS (ESI, M+1): m/z=656.2.

### Example 513

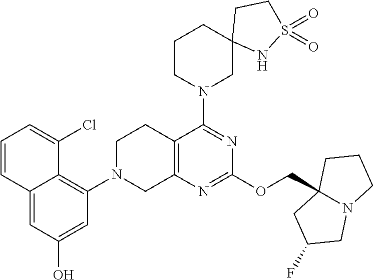 [SMILES: O=S1(=O)CCC2(CCCN(c3nc(OC[C@@]45CCCN4C[C@H](F)C5)nc4c3CCN(c3cc(O)cc5cccc(Cl)c35)C4)C2)N1]

### 7-(7-(8-chloro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-2-thia-1,7-diazaspiro[4.5]decane 2,2-dioxide

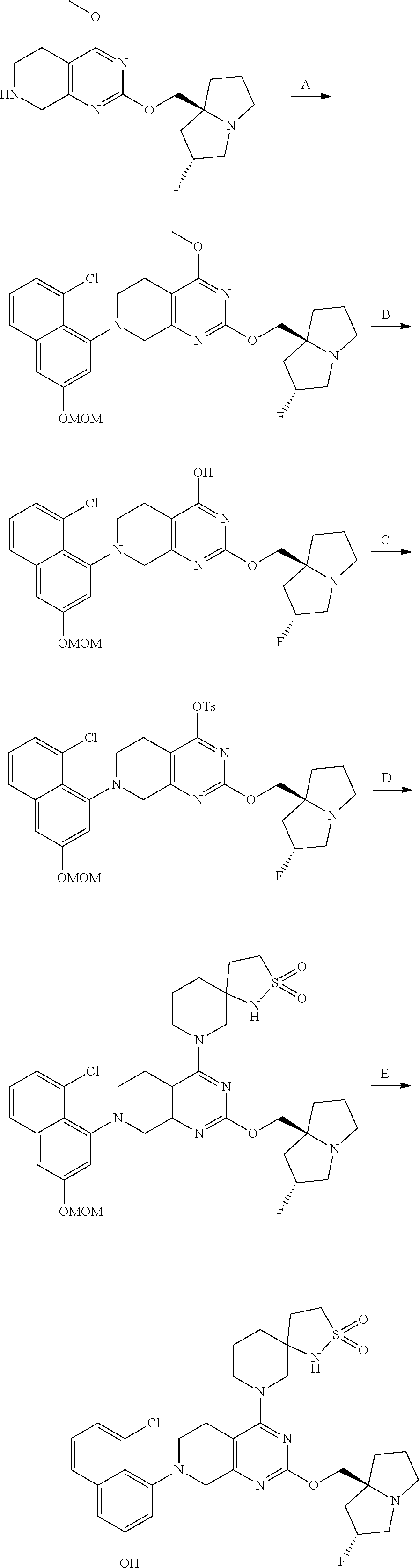

Step A. 7-(8-chloro-3-(methoxymethoxy)naphthalen-1-yl)-2-(((2R,7aS)-2-fluorohexahydro-1H-pyrrolizin-7a-yl)methoxy)-4-methoxy-5,6,7,8-tetrahydropyrido[3,4-d] pyrimidine: To a mixture of 2-(((2R,7aS)-2-fluorohexahydro-1H-pyrrolizin-7a-yl)methoxy)-4-methoxy-5,6,7,8-tetrahydropyrido[3,4-d]pyrimidine (1.30 g, 1.0 equiv), 1-bromo-8-chloro-3-(methoxymethoxy)naphthalene (1.22 g, 1.0 equiv) and CS₂CO₃ (3.94 g, 3.0 equiv) in dioxane (13 mL) was added Xantphos-Pd-G4 (388 mg, 0.10 equiv). The reaction was stirred at 90° C. for 12 hours under N₂ atmosphere. The reaction was diluted with water (20 mL) and extracted with ethyl acetate (3×30 mL). The combined organic layers were dried over anhydrous sodium sulfate, concentrated, and purified with reversed phase chromatography [C18, 0.1% formic acid condition] to afford the title compound (1.13 g, 50% yield) as yellow solid; LCMS (ESI, M+1): m/z=543.2.

Step B. 7-(8-chloro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-ol: To a solution of EtSH (137 mg, 6.0 equiv) in DMAc (2 mL) was added NaH (58.9 mg, 60% purity, 4.0 equiv) at 10° C. The reaction was stirred at 10° C. for 0.5 hours. A solution of 7-(8-chloro-3-(methoxymethoxy)naphthalen-1-yl)-2-(((2R,7aS)-2-fluorohexahydro-1H-pyrrolizin-7a-yl)methoxy)-4-methoxy-5,6,7,8-tetrahydropyrido[3,4-d]pyrimidine (200 mg, 1.0 equiv) in DMAc (1 mL) was added. The reaction was stirred at 60° C. for 1 hour. The mixture was diluted with water (10 mL) and extracted with ethyl acetate (3×10 mL). The combined organic layers were washed with brine (30 mL), dried over anhydrous sodium sulfate, concentrated to afford the title compound (300 mg, crude) as yellow liquid; LCMS (ESI, M+1): m/z=529.2.

Step C. 7-(8-chloro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate: To a solution of 7-(8-chloro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-ol (300 mg, 1.0 equiv), DIEA (220 mg, 3.0 equiv) and DMAP (6.93 mg, 0.1 equiv) in dichloromethane (2 mL) was added TsCl (162 mg, 1.5 equiv) at 0° C. The reaction was stirred at 20° C. for 2 hours. The reaction was diluted with water (10 mL) and extracted with dichloromethane (3×5 mL). The combined organic layers were dried over anhydrous sodium sulfate, concentrated, and purified with column chromatography [Al₂O₃, Petroleum ether/Ethyl acetate=10/1 to 0/1] to afford the title compound (120 mg, 30% yield) as yellow liquid; LCMS (ESI, M+1): m/z=683.0.

The last two steps were performed according to Example 248. The title compound was obtained as yellow solid (FA salt). ¹H NMR (400 MHz, DMSO-d₆) δ=10.21-9.53 (m, 1H), 7.65 (dt, J=1.6, 4.8 Hz, 1H), 7.32-7.27 (m, 2H), 7.21-7.14 (m, 1H), 6.94 (t, J=2.4 Hz, 1H), 6.89-6.81 (m, 1H), 5.34-5.17 (m, 1H), 4.14 (br dd, J=5.6, 17.2 Hz, 1H), 3.99-3.91 (m, 1H), 3.89-3.81 (m, 1H), 3.66 (br dd, J=3.6, 17.2 Hz, 1H), 3.57-3.50 (m, 2H), 3.45 (br d, J=10.0 Hz, 2H), 3.19-3.04 (m, 7H), 3.03-2.98 (m, 2H), 2.85-2.78 (m, 1H), 2.28-2.06 (m, 3H), 2.04-1.94 (m, 2H), 1.90-1.76 (m, 3H), 1.75-1.58 (m, 4H); LCMS (ESI, M+1): m/z=657.3.

### Example 514

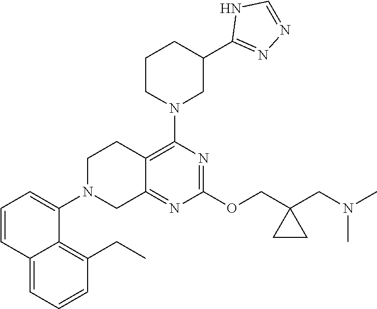 [SMILES: CCc1cccc2cccc(N3CCc4c(nc(OCC5(CN(C)C)CC5)nc4N4CCCC(c5nnc[nH]5)C4)C3)c12]

### 1-(1-(((4-(3-(4H-1,2,4-triazol-3-yl)piperidin-1-yl)-7-(8-ethylnaphthalen-1-yl)-5,6,7,8-tetrahydropyrido[3,4-d]pyrimidin-2-yl)oxy)methyl)cyclopropyl)-N,N-dimethylmethanamine

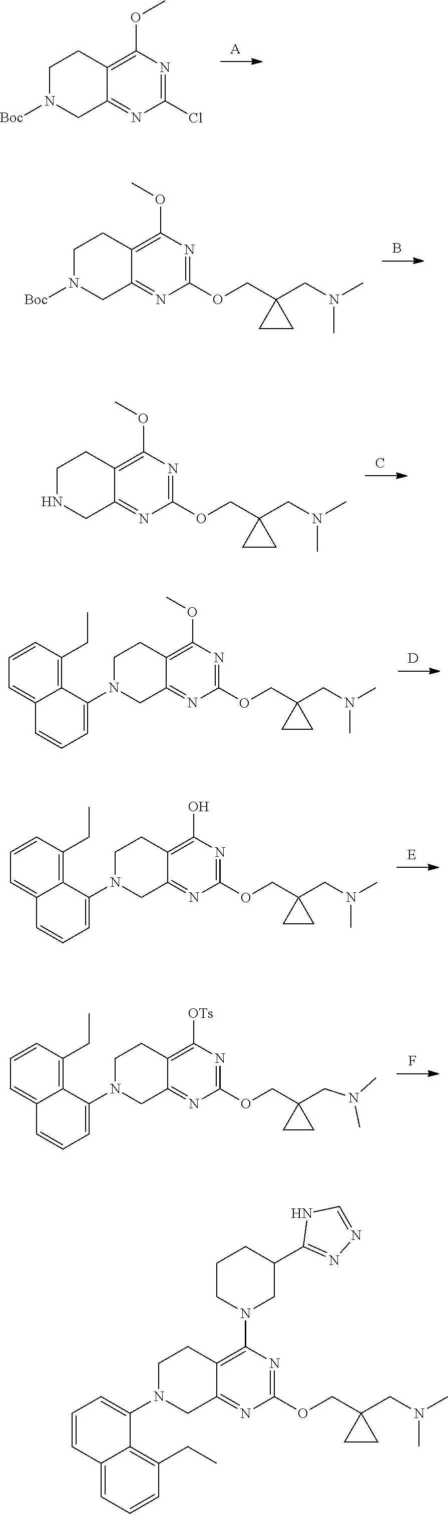

Step A. tert-butyl 2-((1-((dimethylamino)methyl)cyclopropyl)methoxy)-4-methoxy-5,6-dihydropyrido[3,4-d]pyrimidine-7(8H)-carboxylate: To a solution of tert-butyl 2-chloro-4-methoxy-5,6-dihydropyrido[3,4-d]pyrimidine-7(8H)-carboxylate (100 g, 334 mmol, 1.0 equiv) and (1-((dimethylamino)methyl)cyclopropyl)methanol (51.7 g, 400 mmol, 1.2 equiv) in toluene (1000 mL) were added Cs₂CO₃ (326 g, 3.0 equiv) and BINAP (41.6 g, 0.2 equiv). The suspension was degassed under vacuum and purged with N₂ two times, and then Pd(OAc)₂ (7.49 g, 0.1 equiv) was added. The suspension was degassed under vacuum and purged with N₂ three times. The reaction was heated to 110° C. and stirred for 2 hours. The reaction was diluted with water (1000 mL) and extracted with ethyl acetate (3×300 mL). The combined organic layers were washed with brine (1000 mL), dried over Na₂SO₄, concentrated and purified by column chromatography (Al₂O₃, Petroleum ether: Ethyl acetate=I/O to 20:1) to give the title compound (114 g, 87% yield) as yellow oil. ¹H NMR (400 MHz, dimethylsulfoxide-d₆) δ=4.32 (br s, 2H), 4.10 (s, 2H), 3.90 (s, 3H), 3.55 (br s, 2H), 2.49-2.44 (m, 2H), 2.19 (s, 2H), 2.14 (s, 6H), 1.42 (s, 9H), 0.63-0.56 (m, 2H), 0.42-0.33 (m, 2H). LCMS [ESI, M+1]: 393.3.

Step B. 1-(1-(((4-methoxy-5,6,7,8-tetrahydropyrido[3,4-d]pyrimidin-2-yl)oxy)methyl)cyclopropyl)-N,N-dimethylmethanamine: To a solution of tert-butyl 2-((1-((dimethylamino)methyl)cyclopropyl)methoxy)-4-methoxy-5,6-dihydropyrido[3,4-d]pyrimidine-7(8H)-carboxylate (75 g, 1.0 equiv) in DCM (300 mL) was added dropwise TFA (462 g, 300 mL, 21.2 equiv) with stirred at 0° C. The reaction was stirred at 20° C. for 2 hours. The mixture was concentrated, and its pH was adjusted to 10 with saturated Na₂CO₃ aqueous solution and 15% NaOH aqueous solution. Then the mixture was extracted with dichloromethane:methanol (10:1, 800 mL×3), dried over Na₂SO₄, and concentrated to afford the title compound (110 g, 98% yield) as white solid. ¹H NMR (400 MHz, dimethylsulfoxide-d₆) δ=4.09 (s, 2H), 3.88 (s, 3H), 3.62 (s, 2H), 2.89 (t, J=5.6 Hz, 2H), 2.37 (br t, J=5.6 Hz, 2H), 2.20 (s, 2H), 2.15 (s, 6H), 0.63-0.50 (m, 2H), 0.44-0.33 (m, 2H). LCMS [ESI, M+1]: 293.0

Step C. 1-(1-(((7-(8-ethylnaphthalen-1-yl)-4-methoxy-5,6,7,8-tetrahydropyrido[3,4-d]pyrimidin-2-yl)oxy)methyl)cyclopropyl)-N,N-dimethylmethanamine: To a mixture of 1-(1-(((4-methoxy-5,6,7,8-tetrahydropyrido[3,4-d]pyrimidin-2-yl)oxy)methyl)cyclopropyl)-N,N-dimethylmethanamine (20 g, 1.0 equiv), 1-bromo-8-ethylnaphthalene (24.1 g, 1.5 equiv), Cs₂CO₃ (66.9 g, 3.0 equiv) and Xantphos (9.9 g, 0.25 equiv) in toluene (200 mL) was added Pd₂(dba)₃ (6.3 g, 0.15 equiv). The suspension was degassed under vacuum and purged with N₂ several times. The reaction was stirred at 110° C. for 12 hours. The mixture was diluted with water (200 mL) and extracted with ethyl acetate (3×100 mL). The combined organic layers were dried over anhydrous sodium sulfate, concentrated, and purified by column chromatography (SiO2, Petroleum ether: Ethyl acetate=I/O to 1:1) to afford the title compound (26.5 g, 86% yield) as white solid; ¹H NMR (400 MHz, CHLOROFORM-d) δ=7.75-7.62 (m, 2H), 7.45-7.33 (m, 2H), 7.29-7.26 (m, 2H), 4.22 (s, 2H), 4.16-4.07 (m, 1H), 4.04 (s, 3H), 3.78 (d, J=17.2 Hz, 1H), 3.57-3.49 (m, 1H), 3.48-3.39 (m, 1H), 3.29-3.18 (m, 1H), 3.17-3.03 (m, 1H), 2.93-2.82 (m, 1H), 2.67 (br d, J=16.4 Hz, 1H), 2.37 (s, 2H), 2.27 (s, 6H), 1.11 (t, J=7.2 Hz, 3H), 0.70-0.60 (m, 2H), 0.50-0.41 (m, 2H); LCMS [ESI, M+1]: 447.1.

Step D. 2-((1-((dimethylamino)methyl)cyclopropyl)methoxy)-7-(8-ethylnaphthalen-1-yl)-5,6,7,8-tetrahydropyrido[3,4-d] pyrimidin-4-ol: To a solution of ethanethiol (66.8 g, 4.0 equiv) in N,N-dimethylacetamide (1200 mL) was added NaH (21.5 g, 60% purity, 2.0 equiv) at 10° C. The mixture was stirred at 10° C. for 0.5 hour. Then 1-(1-(((7-(8-ethylnaphthalen-1-yl)-4-methoxy-5,6,7,8-tetrahydropyrido[3,4-d]pyrimidin-2-yl)oxy)methyl)cyclopropyl)-N,N-dimethylmethanamine (120 g, 1.0 equiv) was added. The reaction was stirred at 60° C. for 1 hour. The mixture was diluted with water (2000 mL) at 0° C. and extracted with ethyl acetate (3×500 mL). The combined organic layers were washed with brine (500 mL×3), dried over Na₂SO₄, concentrated to afford the title compound (104 g, crude) as yellow oil; LCMS [ESI, M+1]: 433.1.

Step E. 2-((1-((dimethylamino)methyl)cyclopropyl)methoxy)-7-(8-ethylnaphthalen-1-yl)-5,6,7,8-tetrahydropyrido[3,4-d] pyrimidin-4-vl4-methylbenzenesulfonate:To a solution of 2-((1-((dimethylamino)methyl)cyclopropyl)methoxy)-7-(8-ethylnaphthalen-1-yl)-5,6,7,8-tetrahydropyrido[3,4-d]pyrimidin-4-ol (68 g, 1.0 equiv), DIEA (60.9 g, 3.0 equiv) and DMAP (1.92 g, 0.1 equiv) in DCM (680 mL) was added 4-methylbenzenesulfonyl chloride (45.0 g, 1.5 equiv) at 0° C. The reaction was stirred at 20° C. for 2 hours. The mixture was diluted with water (1000 mL) and extracted with ethyl acetate (3×300 mL). The combined organic layers were dried over anhydrous sodium sulfate, concentrated, and purified by column chromatography (SiO2, Petroleum ether: Ethyl acetate=I/O to 3:1) to afford the title compound (80 g, 85% yield) as brown oil; ¹H NMR (400 MHz, dimethylsulfoxide-d₆) 6=8.05-7.94 (m, 2H), 7.81-7.69 (m, 2H), 7.52 (br d, J=7.6 Hz, 2H), 7.46 (br t, J=7.6 Hz, 1H), 7.42-7.34 (m, 2H), 7.32-7.25 (m, 1H), 4.01-3.79 (m, 4H), 3.52-3.41 (m, 1H), 3.29-3.20 (m, 2H), 3.09-2.97 (m, 1H), 2.93-2.80 (m, 1H), 2.77-2.64 (m, 1H), 2.44 (s, 3H), 2.20-2.05 (m, 8H), 1.05-0.96 (m, 3H), 0.53 (br s, 2H), 0.37 (br s, 2H); LCMS [ESI, M+1]: 587.1.

Step F. 1-(1-(((4-(3-(4H-1,2,4-triazol-3-yl)piperidin-1-yl)-7-(8-ethylnaphthalen-1-yll-5,6,7,8-tetrahydropyrido[3,4-d] pyrimidin-2-yl)oxy)methyl)cyclopropyl)-N,N-dimethylmethanamine: A mixture of 2-((1-((dimethylamino)methyl)cyclopropyl)methoxy)-7-(8-ethylnaphthalen-1-yl)-5,6,7,8-tetrahydropyrido[3,4-d]pyrimidin-4-yl 4-methylbenzenesulfonate (0.0264 g, 44.9 mol, 1 eqv), 3-(4H-1,2,4-triazol -3-yl)piperi dine dihydrochloride (0.0203 g, 90.9 μmol, 2 eqv.) and ethylbis(propan-2-yl)amine (0.035 g, 271.3 μmol, 6 eqv.) in acetonitrile (1 mL) was heated with stirring at 50° C. for 16 hours. The resulting solution was cooled to room temperature and subjected to HPLC purification (deionized water/HPLC-grade acetonitrile, ammonia) to give the tile compound as yellow solid (0.0097 g, 17.1 tmol, 98.27%˜purity by LCMS, 38.1% yield, LCMS [M+1]: 567.4).

Example 515 to 573 were synthesized according to Example 514.

| Example No. | Observed M + 1 |
|---|---|
| 515 | 560.2 |
| 516 | 544.2 |
| 517 | 569.2 |
| 518 | 555.4 |
| 519 | 553.2 |
| 520 | 583.2 |
| 521 | 558.2 |
| 522 | 581.4 |
| 523 | 596.4 |
| 524 | 617.4 |
| 525 | 613.4 |
| 526 | 556.4 |
| 527 | 619.2 |
| 528 | 638.4 |
| 529 | 596.2 |
| 530 | 591.2 |
| 531 | 583.2 |
| 532 | 599.2 |
| 533 | 568.2 |
| 534 | 566.2 |
| 535 | 580.4 |
| 536 | 611.4 |
| 537 | 542.3 |
| 538 | 583.2 |
| 539 | 542.4 |
| 540 | 600.4 |
| 541 | 588.2 |
| 542 | 552.4 |
| 543 | 548.4 |
| 544 | 566.2 |
| 545 | 623.4 |
| 546 | 588.2 |
| 547 | 571.2 |
| 548 | 649.3 |
| 549 | 569.3 |
| 550 | 570.2 |
| 551 | 543.2 |
| 552 | 595.4 |
| 553 | 596.4 |
| 554 | 554.4 |
| 555 | 544.4 |
| 556 | 589.2 |
| 557 | 585.2 |
| 558 | 540.4 |
| 559 | 572.4 |
| 560 | 532.2 |
| 561 | 579.2 |
| 562 | 612.4 |
| 563 | 597.2 |
| 564 | 600.4 |
| 565 | 605.2 |
| 566 | 610.2 |
| 567 | 566.2 |
| 568 | 573.2 |
| 569 | 605.4 |
| 570 | 567.2 |
| 571 | 569.4 |
| 572 | 577.2 |
| 573 | 631.4 |

### Example 515

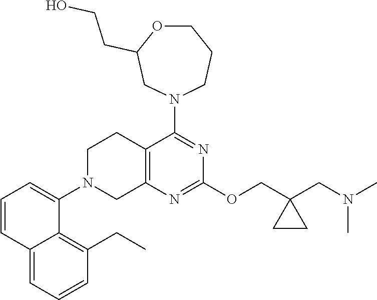 [SMILES: CCc1cccc2cccc(N3CCc4c(nc(OCC5(CN(C)C)CC5)nc4N4CCCOC(CCO)C4)C3)c12]

### 2-(4-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)-1,4-oxazepan-2-yl)ethan-1-ol

### Example 516

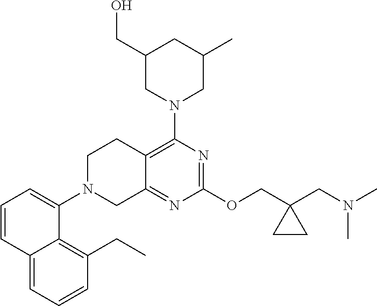 [SMILES: CCc1cccc2cccc(N3CCc4c(nc(OCC5(CN(C)C)CC5)nc4N4CC(C)CC(CO)C4)C3)c12]

### (1-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)-5-methylpiperidin-3-yl)methanol

### Example 517

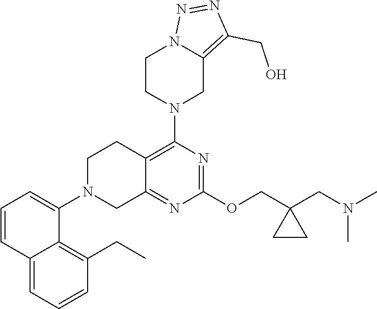 [SMILES: CCc1cccc2cccc(N3CCc4c(nc(OCC5(CN(C)C)CC5)nc4N4CCn5nnc(CO)c5C4)C3)c12]

### (5-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)-4,5,6,7-tetrahydro-[1,2,3]triazolo[1,5-a]pyrazin-3-yl)methanol

### Example 518

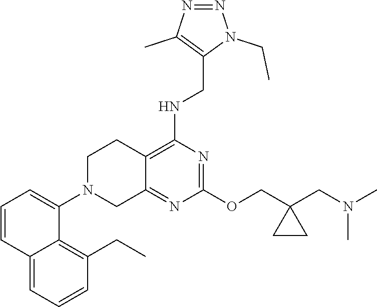 [SMILES: CCc1cccc2cccc(N3CCc4c(nc(OCC5(CN(C)C)CC5)nc4NCc4c(C)nnn4CC)C3)c12]

### 2-((1-((dimethylamino)methyl)cyclopropyl)methoxy)-N-((1-ethyl-4-methyl-1H-1,2,3-triazol-5-yl)methyl)-7-(8-ethylnaphthalen-1-yl)-5,6,7,8-tetrahydropyrido[3,4-d]pyrimidin-4-amine Example 519

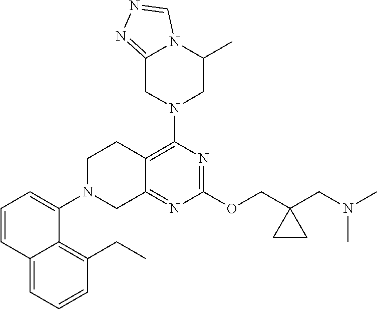 [SMILES: CCc1cccc2cccc(N3CCc4c(nc(OCC5(CN(C)C)CC5)nc4N4Cc5nncn5C(C)C4)C3)c12]

### 1-(1-(((7-(8-ethylnaphthalen-1-yl)-4-(5-methyl-5,6-dihydro-[1,2,4]triazolo[4,3-a]pyrazin-7(8H)-yl)-5,6,7,8-tetrahydropyrido[3,4-d]pyrimidin-2-yl)oxy)methyl)cyclopropyl)-N,N-dimethylmethanamine

### Example 520

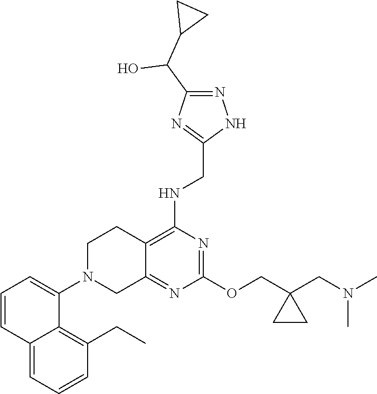 [SMILES: CCc1cccc2cccc(N3CCc4c(nc(OCC5(CN(C)C)CC5)nc4NCc4nc(C(O)C5CC5)n[nH]4)C3)c12]

### cyclopropyl(5-(((2-((1-((dimethylamino)methyl)cyclopropyl)methoxy)-7-(8-ethylnaphthalen-1-yl)-5,6,7,8-tetrahydropyrido[3,4-d]pyrimidin-4-yl)amino)methyl)-1H-1,2,4-triazol-3-yl)methanol

### Example 521

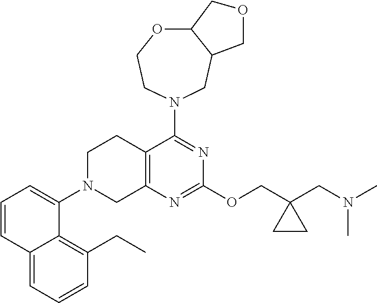 [SMILES: CCc1cccc2cccc(N3CCc4c(nc(OCC5(CN(C)C)CC5)nc4N4CCOC5COCC5C4)C3)c12]

### 1-(1-(((7-(8-ethylnaphthalen-1-yl)-4-(hexahydrofuro[3,4-f][1,4]oxazepin-4(5H)-yl)-5,6,7,8-tetrahydropyrido[3,4-d]pyrimidin-2-yl)oxy)methyl)cyclopropyl)-N,N-dimethylmethanamine

### Example 522

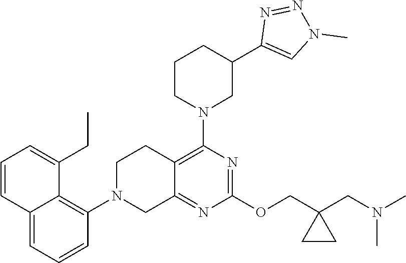 [SMILES: CCc1cccc2cccc(N3CCc4c(nc(OCC5(CN(C)C)CC5)nc4N4CCCC(c5cn(C)nn5)C4)C3)c12]

### 1-(1-(((7-(8-ethylnaphthalen-1-yl)-4-(3-(1-methyl-1H-1,2,3-triazol-4-yl)piperidin-1-yl)-5,6,7,8-tetrahydropyrido[3,4-d]pyrimidin-2-yl)oxy)methyl)cyclopropyl)-N,N-dimethylmethanamine

### Example 523

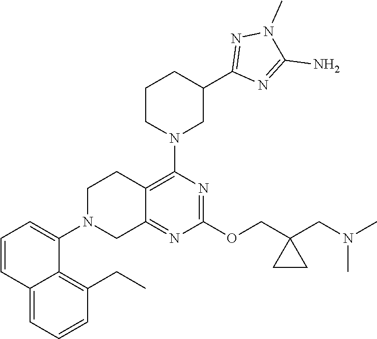 [SMILES: CCc1cccc2cccc(N3CCc4c(nc(OCC5(CN(C)C)CC5)nc4N4CCCC(c5nc(N)n(C)n5)C4)C3)c12]

### 3-(1-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)piperidin-3-yl)-1-methyl-1H-1,2,4-triazol-5-amine

### Example 524

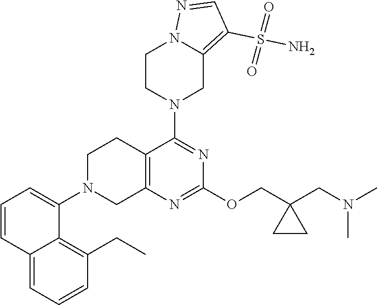 [SMILES: CCc1cccc2cccc(N3CCc4c(nc(OCC5(CN(C)C)CC5)nc4N4CCn5ncc(S(N)(=O)=O)c5C4)C3)c12]

### 5-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)-4,5,6,7-tetrahydropyrazolo[1,5-a]pyrazine-3-sulfonamide

### Example 525

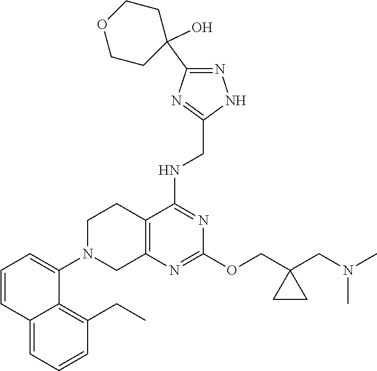 [SMILES: CCc1cccc2cccc(N3CCc4c(nc(OCC5(CN(C)C)CC5)nc4NCc4nc(C5(O)CCOCC5)n[nH]4)C3)c12]

### 4-(5-(((2-((1-((dimethylamino)methyl)cyclopropyl)methoxy)-7-(8-ethylnaphthalen-1-yl)-5,6,7,8-tetrahydropyrido[3,4-d]pyrimidin-4-yl)amino)methyl)-1H-1,2,4-triazol-3-yl)tetrahydro-2H-pyran-4-ol

### Example 526

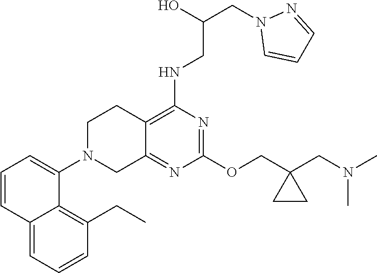 [SMILES: CCc1cccc2cccc(N3CCc4c(nc(OCC5(CN(C)C)CC5)nc4NCC(O)Cn4cccn4)C3)c12]

### 1-((2-((1-((dimethylamino)methyl)cyclopropyl)methoxy)-7-(8-ethylnaphthalen-1-yl)-5,6,7,8-tetrahydropyrido[3,4-d]pyrimidin-4-yl)amino)-3-(1H-pyrazol-1-yl)propan-2-ol

### Example 527

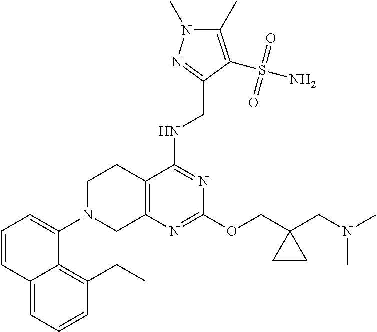 [SMILES: CCc1cccc2cccc(N3CCc4c(nc(OCC5(CN(C)C)CC5)nc4NCc4nn(C)c(C)c4S(N)(=O)=O)C3)c12]

### 3-(((2-((1-((dimethylamino)methyl)cyclopropyl)methoxy)-7-(8-ethylnaphthalen-1-yl)-5,6,7,8-tetrahydropyrido[3,4-d]pyrimidin-4-yl)amino)methyl)-1,5-dimethyl-1H-pyrazole-4-sulfonamide

### Example 528

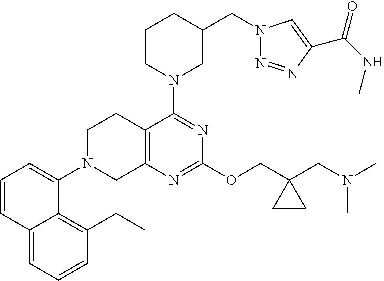 [SMILES: CCc1cccc2cccc(N3CCc4c(nc(OCC5(CN(C)C)CC5)nc4N4CCCC(Cn5cc(C(=O)NC)nn5)C4)C3)c12]

### 1-((1-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)piperidin-3-yl)methyl)-N-methyl-1H-1,2,3-triazole-4-carboxamide

### Example 529

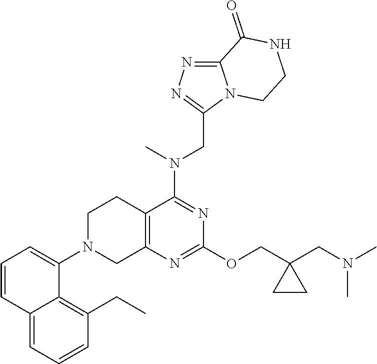 [SMILES: CCc1cccc2cccc(N3CCc4c(nc(OCC5(CN(C)C)CC5)nc4N(C)Cc4nnc5n4CCNC5=O)C3)c12]

### 3-(((2-((1-((dimethylamino)methyl)cyclopropyl)methoxy)-7-(8-ethylnaphthalen-1-yl)-5,6,7,8-tetrahydropyrido[3,4-d]pyrimidin-4-yl)(methyl)amino)methyl)-6,7-dihydro-[1,2,4]triazolo[4,3-a]pyrazin-8(5H)-one

### Example 530

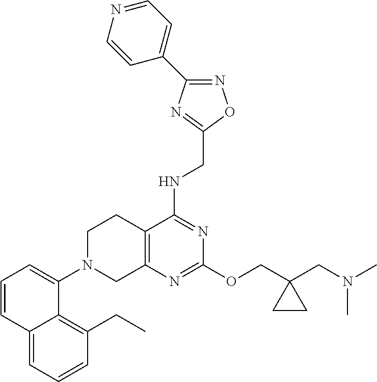 [SMILES: CCc1cccc2cccc(N3CCc4c(nc(OCC5(CN(C)C)CC5)nc4NCc4nc(-c5ccncc5)no4)C3)c12]

### 2-((1-((dimethylamino)methyl)cyclopropyl)methoxy)-7-(8-ethylnaphthalen-1-yl)-N-((3-(pyridin-4-yl)-1,2,4-oxadiazol-5-yl)methyl)-5,6,7,8-tetrahydropyrido[3,4-d]pyrimidin-4-amine

### Example 531

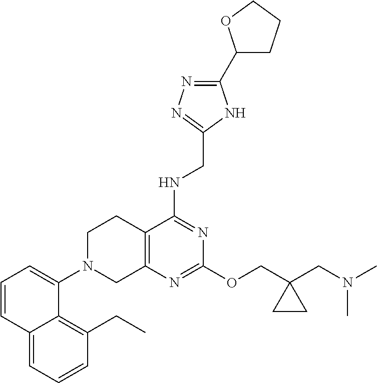 [SMILES: CCc1cccc2cccc(N3CCc4c(nc(OCC5(CN(C)C)CC5)nc4NCc4nnc(C5CCCO5)[nH]4)C3)c12]

### 2-((1-((dimethylamino)methyl)cyclopropyl)methoxy)-7-(8-ethylnaphthalen-1-yl)-N-((5-(tetrahydrofuran-2-yl)-4H-1,2,4-triazol-3-yl)methyl)-5,6,7,8-tetrahydropyrido[3,4-d]pyrimidin-4-amine

### Example 532

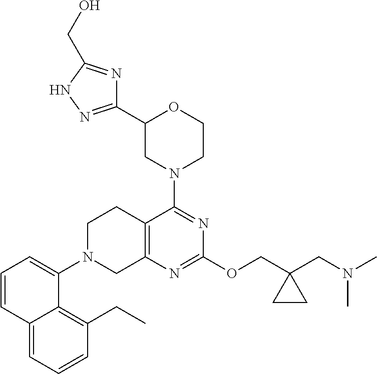 [SMILES: CCc1cccc2cccc(N3CCc4c(nc(OCC5(CN(C)C)CC5)nc4N4CCOC(c5n[nH]c(CO)n5)C4)C3)c12]

### (3-(4-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)morpholin-2-yl)-1H-1,2,4-triazol-5-yl)methanol

### Example 533

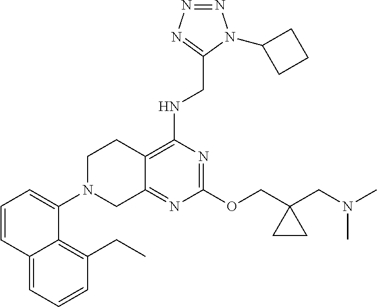 [SMILES: CCc1cccc2cccc(N3CCc4c(nc(OCC5(CN(C)C)CC5)nc4NCc4nnnn4C4CCC4)C3)c12]

N-((1-cyclobutyl-1H-tetrazol-5-yl)methyl)-2-((1-((dimethylamino)methyl)cyclopropyl)methoxy)-7-(8-ethylnaphthalen-1-yl)-5,6,7,8-tetrahydropyrido[3,4-d]pyrimidin-4-amine

### Example 534

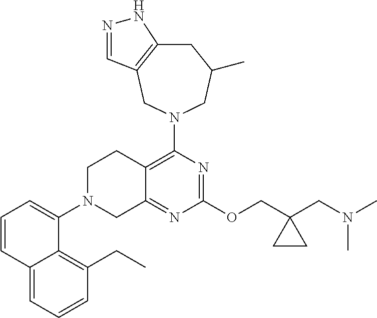 [SMILES: CCc1cccc2cccc(N3CCc4c(nc(OCC5(CN(C)C)CC5)nc4N4Cc5cn[nH]c5CC(C)C4)C3)c12]

### 1-(1-(((7-(8-ethylnaphthalen-1-yl)-4-(7-methyl-4,6,7,8-tetrahydropyrazolo[4,3-c]azepin-5(1H)-yl)-5,6,7,8-tetrahydropyrido[3,4-d]pyrimidin-2-yl)oxy)methyl)cyclopropyl)-N,N-dimethylmethanamine

### Example 535

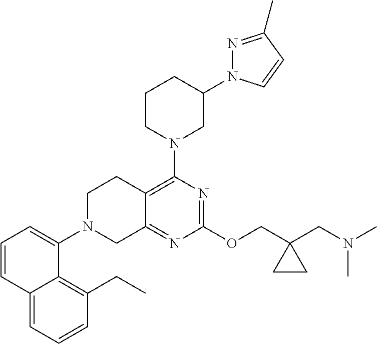 [SMILES: CCc1cccc2cccc(N3CCc4c(nc(OCC5(CN(C)C)CC5)nc4N4CCCC(n5ccc(C)n5)C4)C3)c12]

### 1-(1-(((7-(8-ethylnaphthalen-1-yl)-4-(3-(3-methyl-1H-pyrazol-1-yl)piperidin-1-yl)-5,6,7,8-tetrahydropyrido[3,4-d]pyrimidin-2-yl)oxy)methyl)cyclopropyl)-N,N-dimethylmethanamine

### Example 536

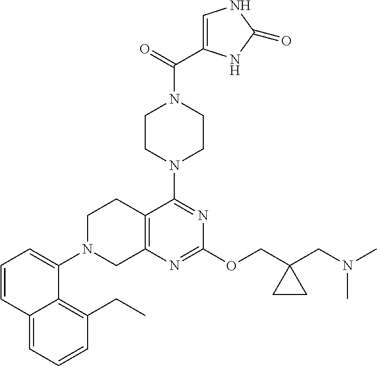 [SMILES: CCc1cccc2cccc(N3CCc4c(nc(OCC5(CN(C)C)CC5)nc4N4CCN(C(=O)c5c[nH]c(=O)[nH]5)CC4)C3)c12]

### 4-(4-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)piperazine-1-carbonyl)-1,3-dihydro-2H-imidazol-2-one

### Example 537

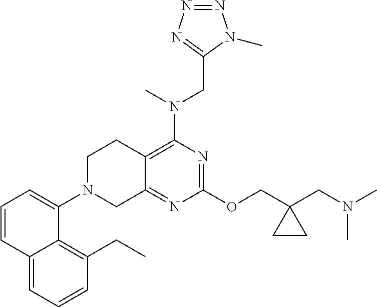 [SMILES: CCc1cccc2cccc(N3CCc4c(nc(OCC5(CN(C)C)CC5)nc4N(C)Cc4nnnn4C)C3)c12]

### 2-((1-((dimethylamino)methyl)cyclopropyl)methoxy)-7-(8-ethylnaphthalen-1-yl)-N-methyl-N-((1-methyl-1H-tetrazol-5-yl)methyl)-5,6,7,8-tetrahydropyrido[3,4-d]pyrimidin-4-amine

### Example 538

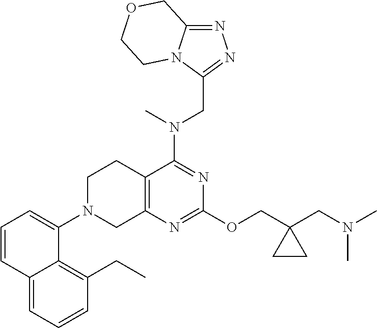 [SMILES: CCc1cccc2cccc(N3CCc4c(nc(OCC5(CN(C)C)CC5)nc4N(C)Cc4nnc5n4CCOC5)C3)c12]

### N-((5,6-dihydro-8H-[1,2,4]triazolo[3,4-c][1,4]oxazin-3-yl)methyl)-2-((1-((dimethylamino)methyl)cyclopropyl)methoxy)-7-(8-ethylnaphthalen-1-yl)-N-methyl-5,6,7,8-tetrahydropyrido[3,4-d]pyrimidin-4-amine

### Example 539

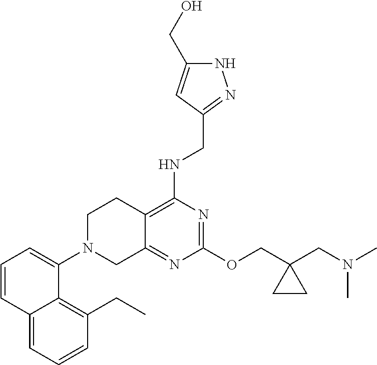 [SMILES: CCc1cccc2cccc(N3CCc4c(nc(OCC5(CN(C)C)CC5)nc4NCc4cc(CO)[nH]n4)C3)c12]

### (3-(((2-((1-((dimethylamino)methyl)cyclopropyl)methoxy)-7-(8-ethylnaphthalen-1-yl)-5,6,7,8-tetrahydropyrido[3,4-d]pyrimidin-4-yl)amino)methyl)-1H-pyrazol-5-yl)methanol

### Example 540

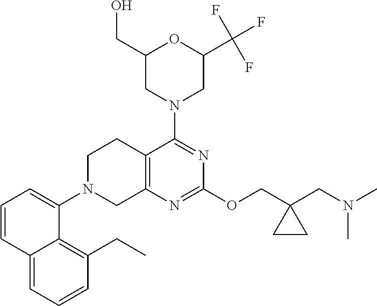 [SMILES: CCc1cccc2cccc(N3CCc4c(nc(OCC5(CN(C)C)CC5)nc4N4CC(CO)OC(C(F)(F)F)C4)C3)c12]

### (4-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)-6-(trifluoromethyl)morpholin-2-yl)methanol

### Example 541

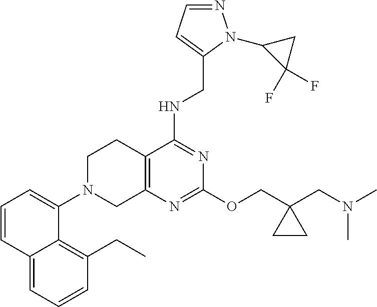 [SMILES: CCc1cccc2cccc(N3CCc4c(nc(OCC5(CN(C)C)CC5)nc4NCc4ccnn4C4CC4(F)F)C3)c12]

### N-((1-(2,2-difluorocyclopropyl)-1H-pyrazol-5-yl)methyl)-2-((1-((dimethylamino)methyl)cyclopropyl)methoxy)-7-(8-ethylnaphthalen-1-yl)-5,6,7,8-tetrahydropyrido[3,4-d]pyrimidin-4-amine

### Example 542

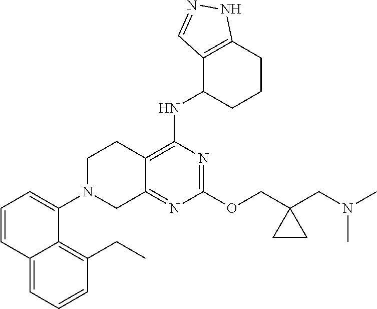 [SMILES: CCc1cccc2cccc(N3CCc4c(nc(OCC5(CN(C)C)CC5)nc4NC4CCCc5[nH]ncc54)C3)c12]

### 2-((1-((dimethylamino)methyl)cyclopropyl)methoxy)-7-(8-ethylnaphthalen-1-yl)-N-(4,5,6,7-tetrahydro-1H-indazol-4-yl)-5,6,7,8-tetrahydropyrido[3,4-d]pyrimidin-4-amine

### Example 543

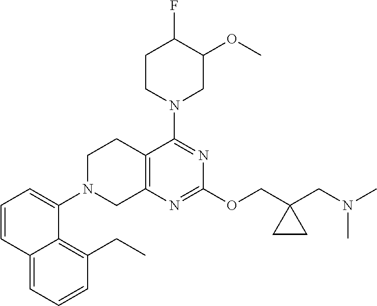 [SMILES: CCc1cccc2cccc(N3CCc4c(nc(OCC5(CN(C)C)CC5)nc4N4CCC(F)C(OC)C4)C3)c12]

### 1-(1-(((7-(8-ethylnaphthalen-1-yl)-4-(4-fluoro-3-methoxypiperidin-1-yl)-5,6,7,8-tetrahydropyrido[3,4-d]pyrimidin-2-yl)oxy)methyl)cyclopropyl)-N,N-dimethylmethanamine

### Example 544

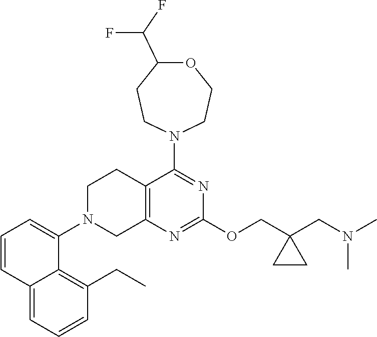 [SMILES: CCc1cccc2cccc(N3CCc4c(nc(OCC5(CN(C)C)CC5)nc4N4CCOC(C(F)F)CC4)C3)c12]

### 1-(1-(((4-(7-(difluoromethyl)-1,4-oxazepan-4-yl)-7-(8-ethylnaphthalen-1-yl)-5,6,7,8-tetrahydropyrido[3,4-d]pyrimidin-2-yl)oxy)methyl)cyclopropyl)-N,N-dimethylmethanamine

### Example 545

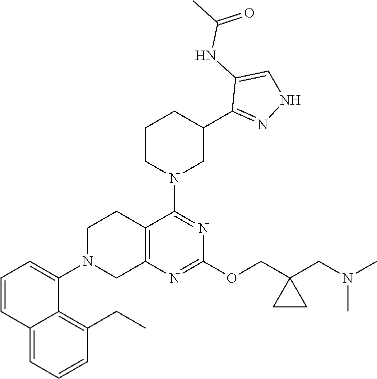 [SMILES: CCc1cccc2cccc(N3CCc4c(nc(OCC5(CN(C)C)CC5)nc4N4CCCC(c5n[nH]cc5NC(C)=O)C4)C3)c12]

### N-(3-(1-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)piperidin-3-yl)-1H-pyrazol-4-yl)acetamide

### Example 546

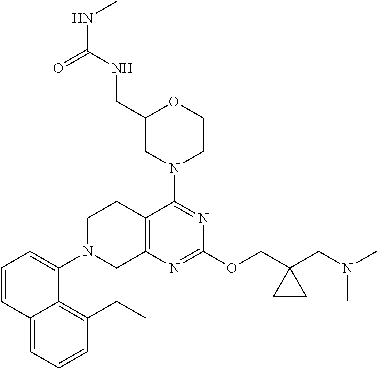 [SMILES: CCc1cccc2cccc(N3CCc4c(nc(OCC5(CN(C)C)CC5)nc4N4CCOC(CNC(=O)NC)C4)C3)c12]

### 1-((4-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)morpholin-2-yl)methyl)-3-methylurea

### Example 547

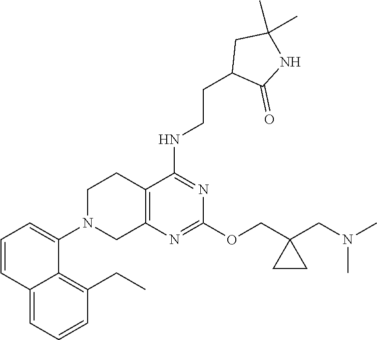 [SMILES: CCc1cccc2cccc(N3CCc4c(nc(OCC5(CN(C)C)CC5)nc4NCCC4CC(C)(C)NC4=O)C3)c12]

### 3-(2-((2-((1-((dimethylamino)methyl)cyclopropyl)methoxy)-7-(8-ethylnaphthalen-1-yl)-5,6,7,8-tetrahydropyrido[3,4-d]pyrimidin-4-yl)amino)ethyl)-5,5-dimethylpyrrolidin-2-one

### Example 548

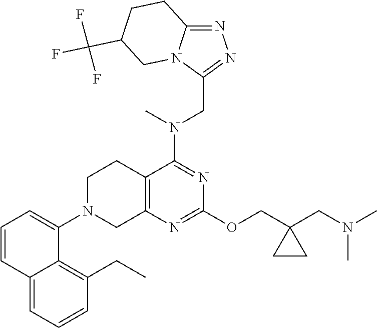 [SMILES: CCc1cccc2cccc(N3CCc4c(nc(OCC5(CN(C)C)CC5)nc4N(C)Cc4nnc5n4CC(C(F)(F)F)CC5)C3)c12]

### 2-((1-((dimethylamino)methyl)cyclopropyl)methoxy)-7-(8-ethylnaphthalen-1-yl)-N-methyl-N-((6-(trifluoromethyl)-5,6,7,8-tetrahydro-[1,2,4]triazolo[4,3-a]pyridin-3-yl)methyl)-5,6,7,8-tetrahydropyrido[3,4-d]pyrimidin-4-amine

### Example 549

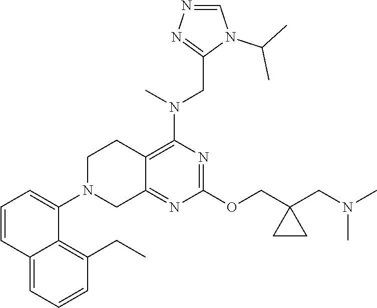 [SMILES: CCc1cccc2cccc(N3CCc4c(nc(OCC5(CN(C)C)CC5)nc4N(C)Cc4nncn4C(C)C)C3)c12]

### 2-((1-((dimethylamino)methyl)cyclopropyl)methoxy)-7-(8-ethylnaphthalen-1-yl)-N-((4-isopropyl-4H-1,2,4-triazol-3-yl)methyl)-N-methyl-5,6,7,8-tetrahydropyrido[3,4-d]pyrimidin-4-amine

### Example 550

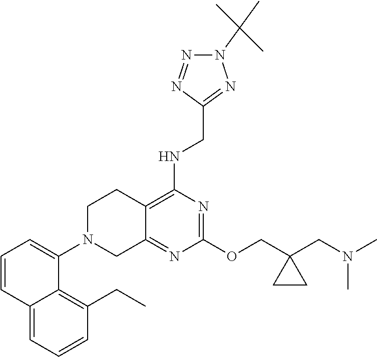 [SMILES: CCc1cccc2cccc(N3CCc4c(nc(OCC5(CN(C)C)CC5)nc4NCc4nnn(C(C)(C)C)n4)C3)c12]

### N-((2-(tert-butyl)-2H-tetrazol-5-yl)methyl)-2-((1-((dimethylamino)methyl)cyclopropyl)methoxy)-7-(8-ethylnaphthalen-1-yl)-5,6,7,8-tetrahydropyrido[3,4-d]pyrimidin-4-amine

### Example 551

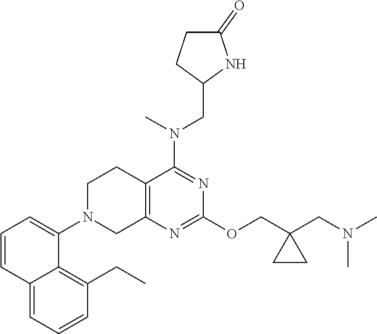 [SMILES: CCc1cccc2cccc(N3CCc4c(nc(OCC5(CN(C)C)CC5)nc4N(C)CC4CCC(=O)N4)C3)c12]

### 5-(((2-((1-((dimethylamino)methyl)cyclopropyl)methoxy)-7-(8-ethylnaphthalen-1-yl)-5,6,7,8-tetrahydropyrido[3,4-d]pyrimidin-4-yl)(methyl)amino)methyl)pyrrolidin-2-one

### Example 552

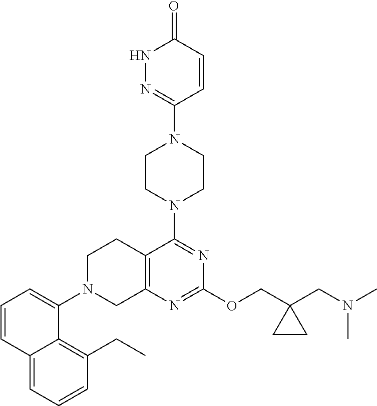 [SMILES: CCc1cccc2cccc(N3CCc4c(nc(OCC5(CN(C)C)CC5)nc4N4CCN(c5ccc(=O)[nH]n5)CC4)C3)c12]

### 6-(4-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)piperazin-1-yl)pyridazin-3(2H)-one

### Example 553

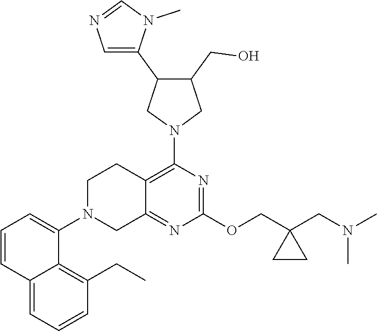 [SMILES: CCc1cccc2cccc(N3CCc4c(nc(OCC5(CN(C)C)CC5)nc4N4CC(CO)C(c5cncn5C)C4)C3)c12]

### (1-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)-4-(1-methyl-1H-imidazol-5-yl)pyrrolidin-3-yl)methanol

### Example 554

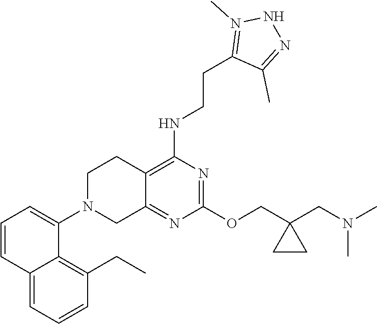

### N-(2-(3,5-dimethyl-1H-pyrazol-4-yl)ethyl)-2-((1-((dimethylamino)methyl)cyclopropyl)methoxy)-7-(8-ethylnaphthalen-1-yl)-5,6,7,8-tetrahydropyrido[3,4-d]pyrimidin-4-amine

### Example 555

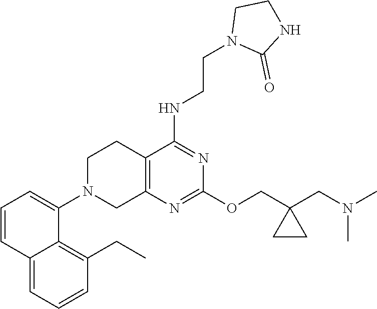 [SMILES: CCc1cccc2cccc(N3CCc4c(nc(OCC5(CN(C)C)CC5)nc4NCCN4CCNC4=O)C3)c12]

### 1-(2-((2-((1-((dimethylamino)methyl)cyclopropyl)methoxy)-7-(8-ethylnaphthalen-1-yl)-5,6,7,8-tetrahydropyrido[3,4-d]pyrimidin-4-yl)amino)ethyl)imidazolidin-2-one

### Example 556

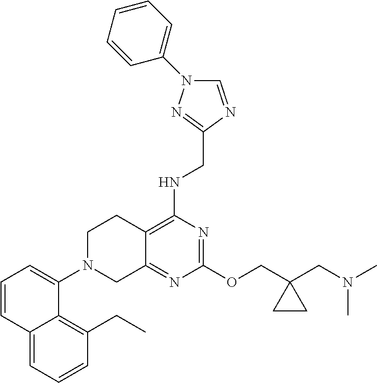 [SMILES: CCc1cccc2cccc(N3CCc4c(nc(OCC5(CN(C)C)CC5)nc4NCc4ncn(-c5ccccc5)n4)C3)c12]

### 2-((1-((dimethylamino)methyl)cyclopropyl)methoxy)-7-(8-ethylnaphthalen-1-yl)-N-((1-phenyl-1H-1,2,4-triazol-3-yl)methyl)-5,6,7,8-tetrahydropyrido[3,4-d]pyrimidin-4-amine

### Example 557

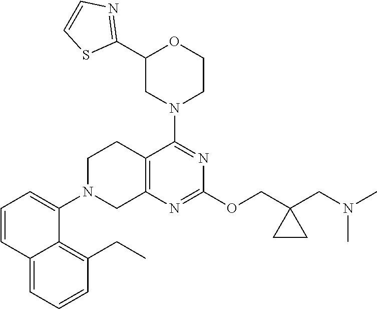 [SMILES: CCc1cccc2cccc(N3CCc4c(nc(OCC5(CN(C)C)CC5)nc4N4CCOC(c5nccs5)C4)C3)c12]

### rel-(R)-1-(1-(((7-(8-(dimenaphthalen-1-yl)-4-(2-(thiazol-2-yl)morpholino)-5,6,7,8-tetrahydropyrido[3,4-d]pyrimidin-2-yl)oxy)methyl)cyclopropyl)-N,N-dimethylmethanamine

### Example 558

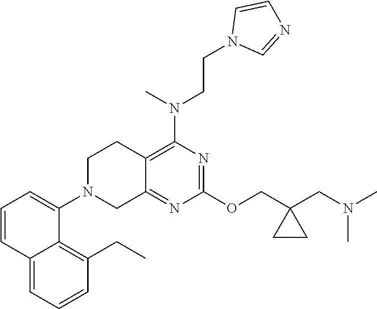 [SMILES: CCc1cccc2cccc(N3CCc4c(nc(OCC5(CN(C)C)CC5)nc4N(C)CCn4ccnc4)C3)c12]

### N-(2-(1H-imidazol-1-yl)ethyl)-2-((1-((dimethylamino)methyl)cyclopropyl)methoxy)-7-(8-ethylnaphthalen-1-yl)-N-methyl-5,6,7,8-tetrahydropyrido[3,4-d]pyrimidin-4-amine

### Example 559

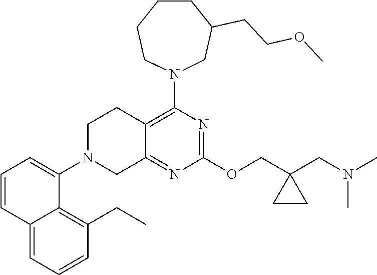 [SMILES: CCc1cccc2cccc(N3CCc4c(nc(OCC5(CN(C)C)CC5)nc4N4CCCCC(CCOC)C4)C3)c12]

### 1-(1-(((7-(8-ethylnaphthalen-1-yl)-4-(3-(2-methoxyethyl)azepan-1-yl)-5,6,7,8-tetrahydropyrido[3,4-d]pyrimidin-2-yl)oxy)methyl)cyclopropyl)-N,N-dimethylmethanamine

### Example 560

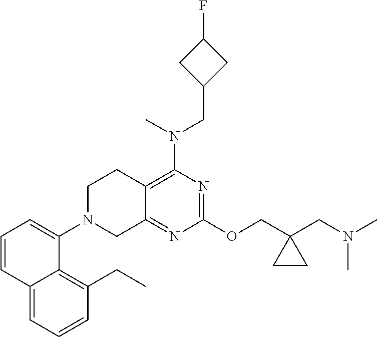 [SMILES: CCc1cccc2cccc(N3CCc4c(nc(OCC5(CN(C)C)CC5)nc4N(C)CC4CC(F)C4)C3)c12]

### 2-((1-((dimethylamino)methyl)cyclopropyl)methoxy)-7-(8-ethylnaphthalen-1-yl)-N-((3-fluorocyclobutyl)methyl)-N-methyl-5,6,7,8-tetrahydropyrido[3,4-d]pyrimidin-4-amine

### Example 561

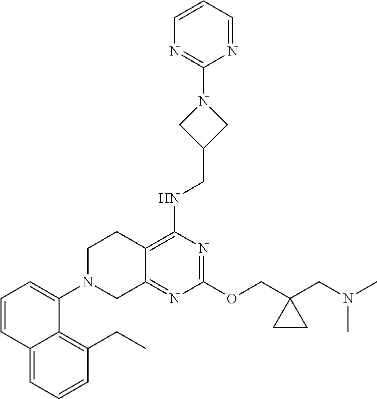 [SMILES: CCc1cccc2cccc(N3CCc4c(nc(OCC5(CN(C)C)CC5)nc4NCC4CN(c5ncccn5)C4)C3)c12]

### 2-((1-((dimethylamino)methyl)cyclopropyl)methoxy)-7-(8-ethylnaphthalen-1-yl)-N-((1-(pyrimidin-2-yl)azetidin-3-yl)methyl)-5,6,7,8-tetrahydropyrido[3,4-d]pyrimidin-4-amine

### Example 562

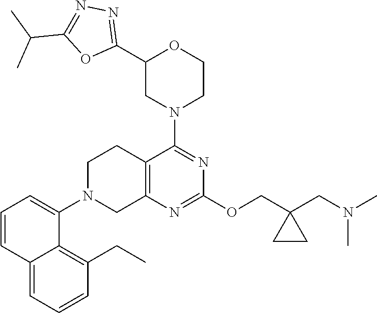 [SMILES: CCc1cccc2cccc(N3CCc4c(nc(OCC5(CN(C)C)CC5)nc4N4CCOC(c5nnc(C(C)C)o5)C4)C3)c12]

### 1-(1-(((7-(8-ethylnaphthalen-1-yl)-4-(2-(5-isopropyl-1,3,4-oxadiazol-2-yl)morpholino)-5,6,7,8-tetrahydropyrido[3,4-d]pyrimidin-2-yl)oxy)methyl)cyclopropyl)-N,N-dimethylmethanamine

### Example 563

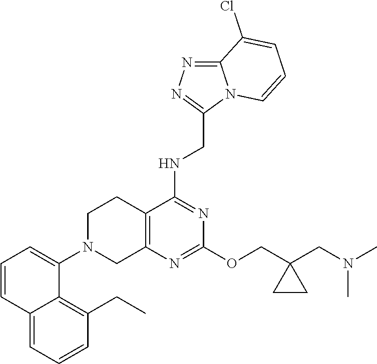 [SMILES: CCc1cccc2cccc(N3CCc4c(nc(OCC5(CN(C)C)CC5)nc4NCc4nnc5c(Cl)cccn45)C3)c12]

### N-((8-chloro-[1,2,4]triazolo[4,3-a]pyridin-3-yl)methyl)-2-((1-((dimethylamino)methyl)cyclopropyl)methoxy)-7-(8-ethylnaphthalen-1-yl)-5,6,7,8-tetrahydropyrido[3,4-d]pyrimidin-4-amine

### Example 564

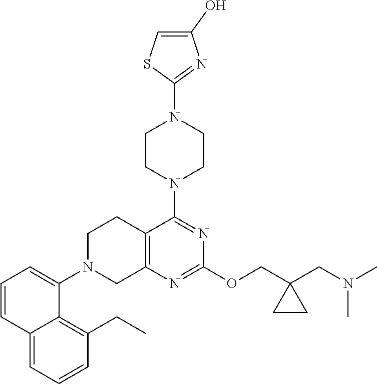 [SMILES: CCc1cccc2cccc(N3CCc4c(nc(OCC5(CN(C)C)CC5)nc4N4CCN(c5nc(O)cs5)CC4)C3)c12]

### 2-(4-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)piperazin-1-yl)thiazol-4-ol

### Example 565

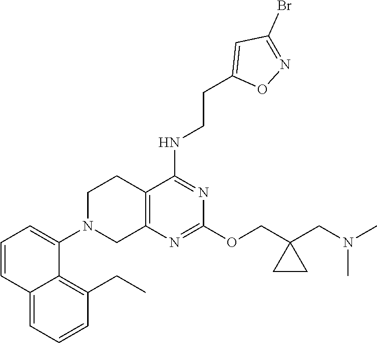 [SMILES: CCc1cccc2cccc(N3CCc4c(nc(OCC5(CN(C)C)CC5)nc4NCCc4cc(Br)no4)C3)c12]

### N-(2-(3-bromoisoxazol-5-yl)ethyl)-2-((1-((dimethylamino)methyl)cyclopropyl)methoxy)-7-(8-ethylnaphthalen-1-yl)-5,6,7,8-tetrahydropyrido[3,4-d]pyrimidin-4-amine

### Example 566

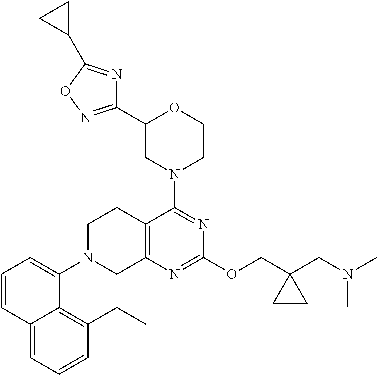 [SMILES: CCc1cccc2cccc(N3CCc4c(nc(OCC5(CN(C)C)CC5)nc4N4CCOC(c5noc(C6CC6)n5)C4)C3)c12]

### 1-(1-(((4-(2-(5-cyclopropyl-1,2,4-oxadiazol-3-yl)morpholino)-7-(8-ethylnaphthalen-1-yl)-5,6,7,8-tetrahydropyrido[3,4-d]pyrimidin-2-yl)oxy)methyl)cyclopropyl)-N,N-dimethylmethanamine

### Example 567

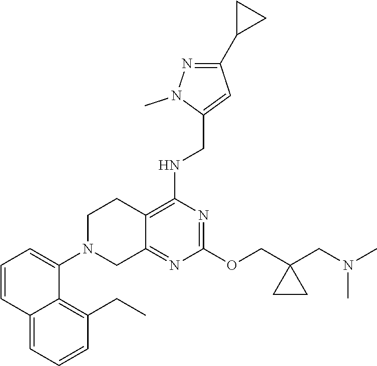 [SMILES: CCc1cccc2cccc(N3CCc4c(nc(OCC5(CN(C)C)CC5)nc4NCc4cc(C5CC5)nn4C)C3)c12]

### N-((3-cyclopropyl-1-methyl-1H-pyrazol-5-yl)methyl)-2-((1-((dimethylamino)methyl)cyclopropyl)methoxy)-7-(8-ethylnaphthalen-1-yl)-5,6,7,8-tetrahydropyrido[3,4-d]pyrimidin-4-amine

### Example 568

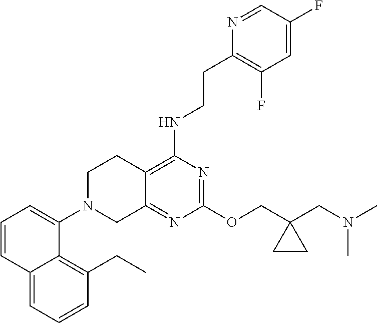 [SMILES: CCc1cccc2cccc(N3CCc4c(nc(OCC5(CN(C)C)CC5)nc4NCCc4ncc(F)cc4F)C3)c12]

### N-(2-(3,5-difluoropyridin-2-yl)ethyl)-2-((1-((dimethylamino)methyl)cyclopropyl)methoxy)-7-(8-ethylnaphthalen-1-yl)-5,6,7,8-tetrahydropyrido[3,4-d]pyrimidin-4-amine

### Example 569

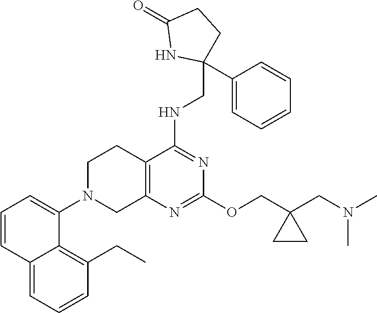 [SMILES: CCc1cccc2cccc(N3CCc4c(nc(OCC5(CN(C)C)CC5)nc4NCC4(c5ccccc5)CCC(=O)N4)C3)c12]

### 5-(((2-((1-((dimethylamino)methyl)cyclopropyl)methoxy)-7-(8-ethylnaphthalen-1-yl)-5,6,7,8-tetrahydropyrido[3,4-d]pyrimidin-4-yl)amino)methyl)-5-phenylpyrrolidin-2-one

### Example 570

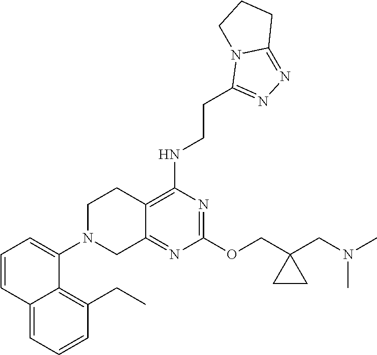 [SMILES: CCc1cccc2cccc(N3CCc4c(nc(OCC5(CN(C)C)CC5)nc4NCCc4nnc5n4CCC5)C3)c12]

### N-(2-(6,7-dihydro-5H-pyrrolo[2,1-c][1,2,4]triazol-3-yl)ethyl)-2-((1-((dimethylamino)methyl)cyclopropyl)methoxy)-7-(8-ethylnaphthalen-1-yl)-5,6,7,8-tetrahydropyrido[3,4-d]pyrimidin-4-amine

### Example 571

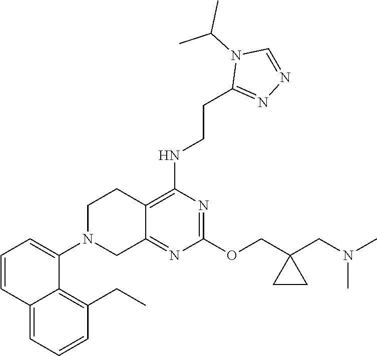 [SMILES: CCc1cccc2cccc(N3CCc4c(nc(OCC5(CN(C)C)CC5)nc4NCCc4nncn4C(C)C)C3)c12]

### 2-((1-((dimethylamino)methyl)cyclopropyl)methoxy)-7-(8-ethylnaphthalen-1-yl)-N-(2-(4-isopropyl-4H-1,2,4-triazol-3-yl)ethyl)-5,6,7,8-tetrahydropyrido[3,4-d]pyrimidin-4-amine

### Example 572

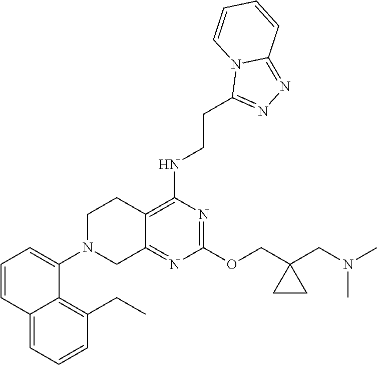 [SMILES: CCc1cccc2cccc(N3CCc4c(nc(OCC5(CN(C)C)CC5)nc4NCCc4nnc5ccccn45)C3)c12]

### N-(2-([1,2,4]triazolo[4,3-a]pyridin-3-yl)ethyl)-2-((1-((dimethylamino)methyl)cyclopropyl)methoxy)-7-(8-ethylnaphthalen-1-yl)-5,6,7,8-tetrahydropyrido[3,4-d]pyrimidin-4-amine

### Example 573

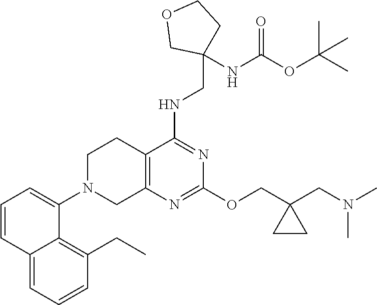 [SMILES: CCc1cccc2cccc(N3CCc4c(nc(OCC5(CN(C)C)CC5)nc4NCC4(NC(=O)OC(C)(C)C)CCOC4)C3)c12]

### tert-butyl (3-(((2-((1-((dimethylamino)methyl)cyclopropyl)methoxy)-7-(8-ethylnaphthalen-1-yl)-5,6,7,8-tetrahydropyrido[3,4-d]pyrimidin-4-yl)amino)methyl)tetrahydrofuran-3-yl)carbamate

### Example 574

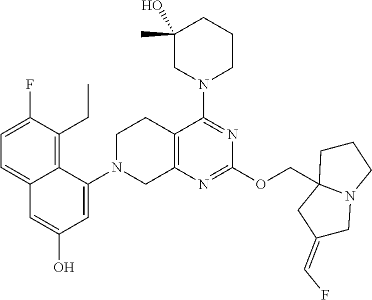 [SMILES: CCc1c(F)ccc2cc(O)cc(N3CCc4c(nc(OCC56CCCN5C/C(=C\F)C6)nc4N4CCC[C@@](C)(O)C4)C3)c12]

### (3R)-1-(7-(8-ethyl-7-fluoro-3-hydroxynaphthalen-1-yl)-2-(((Z)-2-(fluoromethylene)tetrahydro-1H-pyrrolizin-7a(5H)-yl)methoxy)-5,6,7,8-tetrahydropyrido[3,4-d]pyrimidin-4-yl)-3-methylpiperidin-3-ol

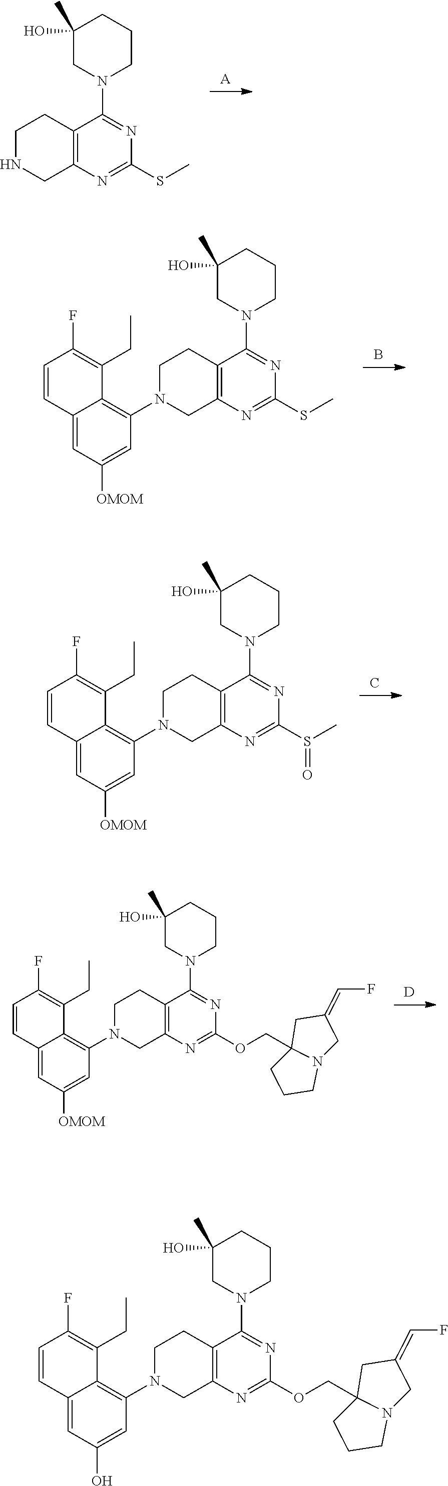

Step A. (R)-1-(7-(8-ethyl-7-fluoro-3-(methoxymethoxy)naphthalen-1-yl)-(methylthio)-5,6,7,8-tetrahydropyrido[3,4-d] pyrimidin-4-yl)-3-methylpiperidin-3-ol: To a mixture of (R)-3-methyl-1-(2-(methylthio)-5,6,7,8-tetrahydropyrido[3,4-d]pyrimidin-4-yl)piperidin-3-ol (1.65 g, 1.0 equiv), 8-ethyl-7-fluoro-3-(methoxymethoxy)naphthalen-1-yl trifluoromethanesulfonate (3.21 g, 1.5 equiv) and Cs₂CO₃ (5.48 g, 3.0equiv) in toluene (16 mL) were added tris(dibenzylideneacetone)dipalladium(0) (513 mg, 0.1 equiv) and Xantphos (649 mg, 0.2 equiv). The reaction was degassed and purged with nitrogen 3 times. The reaction was stirred at 110° C. for 16 hours. The mixture was diluted with water (30 mL) and extracted with ethyl acetate (3×20 mL). The combined organic layers were dried over anhydrous sodium sulfate, filtered, concentrated under vacuum and purified by reversed phase flash [C18, 0.1% formic acid condition] to afford the title compound (600 mg, 17% yield) as red solid;, LCMS (ESI, M+1): m/z=527.2.

Step B. (3R)-1-(7-(8-ethyl-7-fluoro-3-(methoxymethoxy)naphthalen-1-yl)-2-(methylsuliyl)-5,6,7,8-tetrahydropyrido[3,4-d]pyrimidin-4-yl)-3-methylpiperidin-3-ol: To a solution of (R)-1-(7-(8-ethyl -7-fluoro-3-(methoxymethoxy)naphthalen-1I-yl)-2-(methylthi o)-5,6,7,8-tetrahydropyrido [3,4-d]pyrimidin-4-yl)-3-methylpiperidin-3-ol (200 mg, 1.0 equiv) in DCM (2 mL) was added m-CPBA (77.1 mg, 85% purity, 1.0 equiv). The reaction was stirred at 0° C. for 0.5 hours. The mixture was quenched with sodium sulfite saturated aqueous solution (5 mL) and extracted with DCM (3×5 mL). The combined organic layers were dried over anhydrous sodium sulfate, filtered and concentrated in vacuum to afford the title compound (200 mg, 76% yield) as red solid; LCMS (ESI, M+1): m/z=543.2.

Step C. (3R)-1-(7-(8-ethyl-7-fluoro-3-(methoxymethoxy)naphthalen-1-yl)-2-((Z)-2-(fluoromethylene)tetrahydro-1H-pyrrolizin-7a(5H)-yl)methoxy)-5,6,7,8-tetrahydropyrido[3,4-d]pyrimidin-4-yl)-3-methylpiperidin-3-ol: To a solution of (3R)-1-(7-(8-ethyl-7-fluoro-3-(methoxymethoxy)naphthalen-1-yl)-2-(methylsulfinyl)-5,6,7,8-tetrahydropyrido[3,4-d]pyrimidin-4-yl)-3-methylpiperidin-3-ol (200 mg, 1.0 equiv) and (Z)-(2-(fluoromethylene)tetrahydro-1H-pyrrolizin-7a(5H)-yl)methanol (75.7 mg, 1.2 equiv) in THF (2 mL) was added t-BuOK (1 M, 1.5 equiv) at 0° C. The reaction was stirred at 0° C. for 1 hour. The mixture was diluted with water (5 mL) and extracted with ethyl acetate (3×5 mL). The combined organic layers were dried over anhydrous sodium sulfate, filtered and concentrated to afford the title compound (220 mg, 61% yield) as yellow solid; LCMS (ESI, M+1): m/z=650.3.

Step D. (3R)-1-(7-(8-ethyl-7-fluoro-3-hydroxynaphthalen-1-yl)-2-(((Z)-2-(fluoromethylene)tetrahydro-1H-pyrrolizin-7a(5H)-yl)methoxy)-5,6,7,8-tetrahydropyrido[3,4-d]pyrimidin-4-yl)-3-methylpiperidin-3-ol: A solution of (3R)-1-(7-(8-ethyl-7-fluoro-3-(methoxymethoxy)naphthalen-1-yl)-2-(((Z)-2-(fluoromethylene)tetrahydro-1H-pyrrolizin-7a(5H)-yl)methoxy)-5,6,7,8-tetrahydropyrido[3,4-d]pyrimidin-4-yl)-3-methylpiperidin-3-ol (220 mg, 1.0 equiv) in HCl/MeOH (4 M, 26 equiv) was stirred at 20° C. for 0.5 hours. The mixture was concentrated under vacuum. The residue was dissolved in MeOH (2 mL), neutralized with solid NaHCO₃ and filtered. The filtrate was concentrated under vacuum and purified with prep-HPLC (column: Waters xbridge C18 150×25 mm×10 μm;mobile phase: [water(NH₄HCO₃)-ACN];gradient:42%-72% B over 14 min) to afford the title compound (31.9 mg, 15% yield) as yellow solid; ¹H NMR (400 MHz, METHANOL-d₄) δ=7.51 (ddd, J=1.2, 6.0, 8.8 Hz, 1H), 7.14 (t, J=9.6 Hz, 1H), 7.01-6.97 (m, 1H), 6.97-6.94 (m, 1H), 6.76-6.49 (m, 1H), 4.21-4.04 (m, 3H), 3.91-3.79 (m, 1H), 3.72-3.59 (m, 2H), 3.54-3.33 (m, 6H), 3.25-3.05 (m, 4H), 2.77-2.62 (m, 3H), 2.39 (br d, J=16.0 Hz, 1H), 2.12-2.03 (m, 1H), 2.02-1.92 (m, 1H), 1.92-1.86 (m, 1H), 1.85-1.76 (m, 2H), 1.76-1.68 (m, 2H), 1.68-1.59 (m, 1H), 1.28-1.18 (m, 3H), 1.12 (t, J=7.2 Hz, 3H); LCMS (ESI, M+l): m/z=606.3.

### Example 575

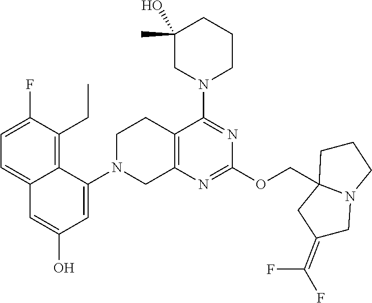 [SMILES: CCc1c(F)ccc2cc(O)cc(N3CCc4c(nc(OCC56CCCN5CC(=C(F)F)C6)nc4N4CCC[C@@](C)(O)C4)C3)c12]

### (3R)-1-(2-((2-(difluoromethylene)tetrahydro-1H-pyrrolizin-7a(5H)-yl)methoxy)-7-(8-ethyl-7-fluoro-3-hydroxynaphthalen-1-yl)-5,6,7,8-tetrahydropyrido[3,4-d]pyrimidin-4-yl)-3-methylpiperidin-3-ol

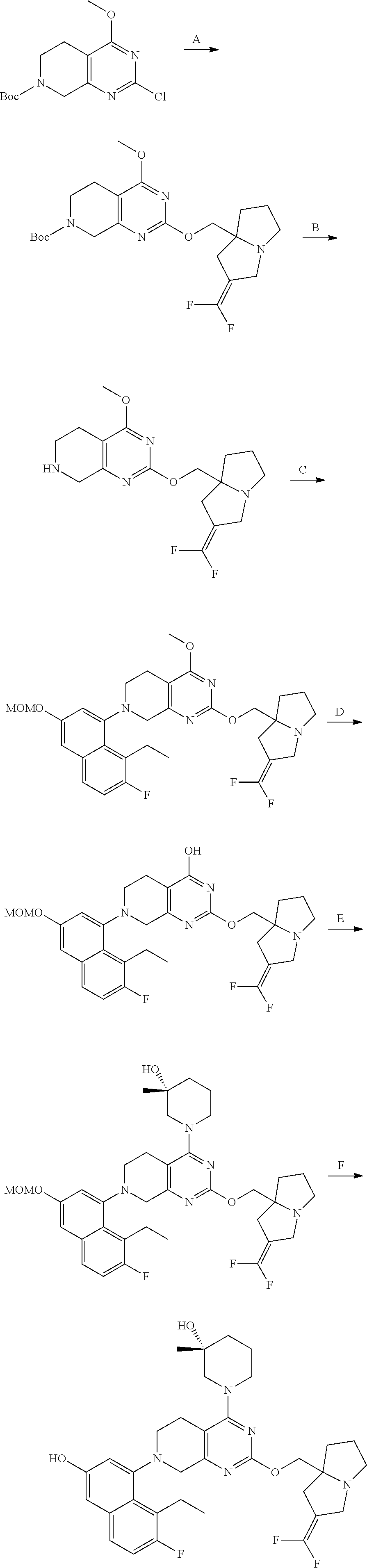

Step A. tert-butyl 2-((2-(difluoromethylene)tetrahydro-1H-pyrrolizin-7a(5H)-yl)methoxy)-4-methoxy-5,8-dihydropyrido[3,4-d] pyrimidine-7(6H)-carboxylate: To a solution of tert-butyl 2-chloro-4-methoxy-5,8-dihydropyrido[3,4-d]pyrimidine-7(6H)-carboxylate (4.00 g, 1.0 equiv) and (2-(difluoromethylene)tetrahydro-1H-pyrrolizin-7a(5H)-yl)methanol (3.28 g, 1.3 equiv) in THE (40 mL) and DMF (40 mL) were added DABCO (1.50 g, 1.0 equiv) and Cs₂CO₃ (13.0 g, 3.0 equiv). The reaction was degassed and purged with N₂ for 3 times. The reaction was stirred at 50° C. for 12 hours under N₂ atmosphere. The mixture was diluted with water (300 mL) and extracted with ethyl acetate (3×200 mL). The combined organic layers were washed with brine (500 mL), dried over anhydrous sodium sulfate, concentrated and purified with column chromatography [SiO2, petroleum ether/ethyl acetate=3/1 to 0/1] to afford the title compound (3.60 g, 52% yield) as brown oil; LCMS (ESI, M+1): m/z=453.3.

Step B. 2-((2-(difluoromethylene)tetrahydro-l1H-pyrrolizin-7a(5H)-yl)methoxy)-4-methoxy-5,6,7,8-tetrahydropyrido[3,4-d] pyrimidine: To a solution of tert-butyl 2-((2-(difluoromethylene)tetrahydro-1H-pyrrolizin-7a(5H)-yl)methoxy)-4-methoxy-5,8-dihydropyrido[3,4-d]pyrimidine-7(6H)-carboxylate (3.60 g, 1.0 equiv) in MeOH (40 mL) was added HCl•MeOH (40 mL). The reaction was stirred at 25° C. for 1 hour. The mixture was diluted with sodium hydroxide solution (1 N, 4×100 mL) and extracted with DCM/MeOH=10/1 (3×80 mL). The combined organic layers were washed with brine (200 mL), dried over anhydrous sodium sulfate, concentrated to afford the title compound (2.55 g, 82% yield) as brown oil; LCMS (ESI, M+1): m/z=353.2.

Step C. 2-((2-(difluoromethylene)tetrahydro-l1H-pyrrolizin-7a(5H)-yl)methoxy)-7-(8-ethyl-7-fluoro-3-(methoxymethoxy)naphthalen-1-yl)-4-methoxy-5,6,7,8-tetrahydropyrido[3,4-d] pyrimidine: To a solution of 2-((2-(difluoromethylene)tetrahydro-1H-pyrrolizin-7a(5H)-yl)methoxy)-4-methoxy-5,6,7,8-tetrahydropyrido[3,4-d]pyrimidine (1.30 g, 1.0 equiv) and 8-ethyl-7-fluoro-3-(methoxymethoxy)naphthalen-1-yl trifluoromethanesulfonate (2.12 g, 1.5 equiv) in dioxane (20 mL) were added Pd₂(dba)₃ (337 mg, 0.1 equiv), Xantphos (426 mg, 0.2 equiv) and Cs₂CO₃ (3.61 g, 3.0 equiv). The reaction was degassed and purged with N₂ for 3 times. The reaction was stirred at 90° C. for 12 hours under N₂ atmosphere. The mixture was diluted with water (50 mL) and extracted with ethyl acetate (3×30 mL). The combined organic layers were washed with brine (50 mL), dried over anhydrous sodium sulfate, concentrated and purified with column chromatography [SiO2, petroleum ether/ethyl acetate=10/1 to 1/1] to afford the title compound (370 mg, 10% yield) as brown solid; LCMS (ESI, M+1): m/z=585.4.

Step D. 2-((2-(difluoromethylene)tetrahydro-l1H-pyrrolizin-7a(5H)-yl)methoxy)-7-(8-ethyl-7-fluoro-3-(methoxymethoxy)naphthalen-1-yl)-5,6,7,8-tetrahydropyrido[3,4-d] pyrimidin-4-ol: To a solution of 2-((2-(difluoromethylene)tetrahydro-1H-pyrrolizin-7a(5H)-yl)methoxy)-7-(8-ethyl-7-fluoro-3-(methoxymethoxy)naphthalen-1-yl)-4-methoxy-5,6,7,8-tetrahydropyrido[3,4-d]pyrimidine (350 mg, 1.0 equiv) in DMAc (5 mL) was added NaSEt (307 mg, 10 equiv). The reaction was stirred at 60° C. for 0.25 hours. The mixture was diluted with water (25 mL), adjusted to pH=7 with 2N HCl and extracted with ethyl acetate (3×30 mL). The combined organic layers were washed with brine (30 mL), dried over anhydrous sodium sulfate, concentrated and purified with prep-HPLC [column: Phenomenex luna C18 150×40 mm×15 μm; mobile phase: water(FA)-ACN; gradient: 20%-50% B over 15 min] to afford the title compound (153 mg, 63% yield) as brown solid; LCMS (ESI, M+1): m/z=571.4.

Step E. (3R)-1-(2-((2-(difluoromethylene)tetrahydro-1H-pyrrolizin-7a(5H)-yl)methoxy)-7-(8-ethyl-7-fluoro-3-(methoxymethoxy)naphthalen-1-yl)-5,6,7,8-tetrahydropyrido[3,4-d]pyrimidin-4-yl)-3-methylpiperidin-3-ol: To a solution of 2-((2-(difluoromethylene)tetrahydro-1H-pyrrolizin-7a(5H)-yl)methoxy)-7-(8-ethyl-7-fluoro-3-(methoxymethoxy)naphthalen-1-yl)-5,6,7,8-tetrahydropyrido[3,4-d]pyrimidin-4-ol (78.0 mg, 1.0 equiv) in DMSO (0.5 mL) were added TEA (69.1 mg, 5.0 equiv) and PYBOP (106 mg, 1.5 equiv). The reaction was stirred at 25° C. for 0.5 hours and then (R)-3-methylpiperidin-3-ol (31.0 mg, 1.5 equiv, HCl) was added. The reaction was stirred at 25° C. for 2.5 hours. The mixture was diluted with water (10 mL) and extracted with ethyl acetate (3×15 mL). The combined organic layers were washed with brine (30 mL), dried over anhydrous sodium sulfate, concentrated and purified with prep-TLC [SiO₂, DCM/MeOH=10/1] to afford the title compound (54.0 mg, 52% yield) as brown solid; LCMS (ESI, M+1): m/z=668.4.

Step F. (3R)-1-(2-((2-(difluoromethylene)tetrahydro-1H-pyrrolizin-7a(5H)-yl)methoxy)-7-(8-ethyl-7-fluoro-3-hydroxynaphthalen-1-yl)-5,6,7,8-tetrahydropyrido[3,4-d] pyrimidin-4-yl)-3-methylpiperidin-3-ol: To a solution of (3R)-1-(2-((2-(difluoromethylene)tetrahydro-1H-pyrrolizin-7a(5H)-yl)methoxy)-7-(8-ethyl-7-fluoro-3-(methoxymethoxy)naphthalen-1-yl)-5,6,7,8-tetrahydropyrido[3,4-d]pyrimidin-4-yl)-3-methylpiperidin-3-ol (49.0 mg, 1.0 equiv) in MeOH (1.0 mL) was added HCl-MeOH (1.0 mL) at 0° C. The reaction was stirred at 0° C. for 1 hour. The mixture was concentrated, dissolved in MeOH (2 mL), adjusted to pH=8 with solid NaHCO₃at 0° C. and filtered. The filtration was concentrated and purified with prep-HPLC [column: Waters Xbridge 150×25 mm×5 μm; mobile phase: water (NH₄HCO₃)-ACN; gradient: 55%-85% B over 9 min] to afford the title compound (4.71 mg, 8% yield, CF₃COOH) as yellow solid; ¹H NMR (400 MHz, METHANOL-d₄) δ=7.51 (dd, J=6.4, 8.0 Hz, 1H), 7.14 (t, J=9.2 Hz, 1H), 7.02-6.94 (m, 2H), 4.29-4.05 (m, 3H), 3.92-3.79 (m, 1H), 3.72-3.61 (m, 2H), 3.57-3.34 (m, 6H), 3.16 (br d, J=8.4 Hz, 3H), 2.82-2.67 (m, 3H), 2.49 (br d, J=15.6 Hz, 1H), 2.15-1.59 (m, 9H), 1.28-1.19 (m, 3H), 1.12 (t, J=7.2 Hz, 3H); LCMS (ESI, M+1): m/z=624.4.

### Example 576

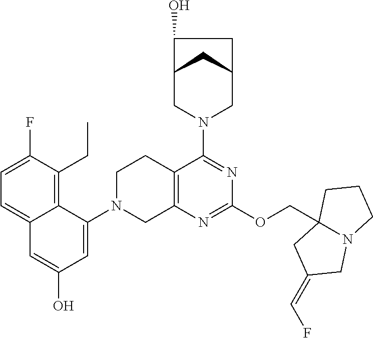 [SMILES: CCc1c(F)ccc2cc(O)cc(N3CCc4c(nc(OCC56CCCN5C/C(=C\F)C6)nc4N4C[C@@H]5C[C@H](C4)[C@H](O)C5)C3)c12]

### (1R,5R,6R)-3-(7-(8-ethyl-7-fluoro-3-hydroxynaphthalen-1-yl)-2-(((Z)-2-(fluoromethylene)tetrahydro-1H-pyrrolizin-7a(5H)-yl)methoxy)-5,6,7,8-tetrahydropyrido[3,4-d]pyrimidin-4-yl)-3-azabicyclo[3.2.1]octan-6-ol

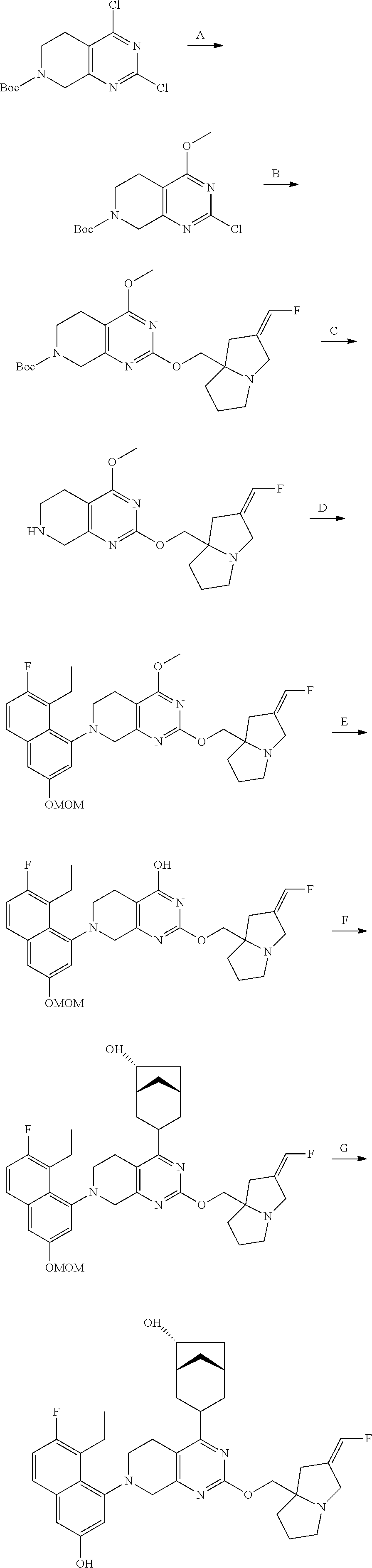

Step A. tert-butyl 2-chloro-4-methoxy-5,8-dihydropyrido[3,4-d]pyrimidine-7(6H)-carboxylate: To a solution of tert-butyl 2,4-dichloro-5,8-dihydropyrido[3,4-d]pyrimidine-7(6H)-carboxylate (10.0 g, 1.0 equiv) in MeOH (10.0 mL) was added NaOMe (2.13 g, 1.2 equiv) at 0° C. The reaction was stirred at 25° C. for 4 hours. The mixture was concentrated and purified by column chromatography (SiO2, Petroleum ether/Ethyl acetate=100/1 to 20/1) to afford the title compound (7.00 g, 71% yield) as yellow oil; ¹H NMR (400 MHz, METHANOL-d₄) δ=4.48 (s, 2H), 4.03 (s, 3H), 3.67 (br t, J=5.6 Hz, 2H), 2.64 (t, J=6.0 Hz, 2H), 1.49 (s, 9H); LCMS (ESI, M+1): m/z=300.0.

Step B. tert-butyl (Z)-2-((2-(fluoromethylene)tetrahydro-1H-pyrrolizin-7a(5H)-yl)methoxy)-4-methoxy-5,8-dihydropyrido[3,4-d]pyrimidine-7(6H)-carboxylate: To a solution of tert-butyl 2-chloro-4-methoxy-5,8-dihydropyrido[3,4-d]pyrimidine-7(6H)-carboxylate (637 mg, 1.0 equiv), (Z)-(2-(fluoromethylene)tetrahydro-1H-pyrrolizin-7a(5H)-yl)methanol (400 mg, 1.1 equiv) and Cs₂CO₃ (2.08 g, 3.0 equiv) in toluene (4.0 mL) were added Pd(OAc)₂ (47.7 mg, 0.10 equiv) and BINAP (264 mg, 0.20 equiv). The reaction was degassed and purged with N₂ for 3 times. The reaction was stirred at 110° C. for 1 hour. The mixture was concentrated and purified by reversed phase flash chromatography [C18, 0.1% formic acid condition] to afford the title compound (550 mg, 59% yield) as yellow solid; LCMS (ESI, M+1): m/z=435.4.

Step C. (Z)-2-((2-(fluoromethylene)tetrahydro-1H-pyrrolizin-7a(5H)-yl)methoxy)-4-methoxy-5,6,7,8-tetrahydropyrido[3,4-d] pyrimidine: A mixture of tert-butyl (Z)-2-((2-(fluoromethylene)tetrahydro-1H-pyrrolizin-7a(5H)-yl)methoxy)-4-methoxy-5,8-dihydropyrido[3,4-d]pyrimidine-7(6H)-carboxylate (550 mg, 1.0 equiv) and HCl•MeOH (4 M, 17.4 equiv) was stirred at 20° C. for 0.5 hours. The mixture was concentrated, diluted with water (3.0 mL), adjusted the pH to 11 with NaOH (4 N) and extracted with ethyl acetate (3×15 mL). The combined organic layers were washed with brine (40 mL), dried over anhydrous sodium sulfate, filtered and concentrated to afford the title compound (520 mg, crude) as yellow solid; LCMS (ESI, M+1): m/z=335.3.

Step D. (Z)-7-(8-ethyl-7-fluoro-3-(methoxymethoxy)naphthalen-1-yl)-2-((2-(fluoromethylene)tetrahydro-1H-pyrrolizin-7a(5H)-yl)methoxy)-4-methoxy-5,6,7,8-tetrahydropyrido[3,4-d]pyrimidine: To a mixture of (Z)-2-((2-(fluoromethylene)tetrahydro-1H-pyrrolizin-7a(5H)-yl)methoxy)-4-methoxy-5,6,7,8-tetrahydropyrido[3,4-d]pyrimidine (50.0 mg, 1.0 equiv), 8-ethyl-7-fluoro-3-(methoxymethoxy)naphthalen-1-yl trifluoromethanesulfonate (114 mg, 2.0 equiv) and Cs₂CO₃ (146 mg, 3.0 equiv) in toluene (1.0 mL) were added Pd₂(dba)₃ (13.7 mg, 0.10 equiv) and Xantphos (17.3 mg, 0.20 equiv). The reaction mixture was degassed and purged with N₂ for 3 times. The reaction was stirred at 110° C. for 12 hours. The mixture was quenched with water (5.0 mL) and extracted with ethyl acetate (3×5.0 mL). The combined organic layers were dried over anhydrous sodium sulfate, concentrated, and purified by reversed phase flash chromatography [C18, 0.1% formic acid condition] to afford the title compound (100 mg, 12% yield) as yellow solid; LCMS (ESI, M+1): m/z=567.5.

Step E. (Z)-7-(8-ethyl-7-fluoro-3-(methoxymethoxy)naphthalen-1-yl)-2-((2-(fluoromethylene)tetrahydro-1H-pyrrolizin-7a(5H)-yl)methoxy)-5,6,7,8-tetrahydropyrido[3,4-d]pyrimidin-4-ol: To a solution of (Z)-7-(8-ethyl-7-fluoro-3-(methoxymethoxy)naphthalen-1-yl)-2-((2-(fluoromethylene)tetrahydro-1H-pyrrolizin-7a(5H)-yl)methoxy)-4-methoxy-5,6,7,8-tetrahydropyrido[3,4-d]pyrimidine (110 mg, 1.0 equiv) in DMAC (1.0 mL) was added NaSEt (49.0 mg, 3.0 equiv). The reaction was stirred at 60° C. for 1 hour. The mixture was quenched with water (5 mL) and extracted with Ethyl acetate (3×5.0 mL). The combined organic layers were dried over anhydrous sodium sulfate and concentrated to afford the title compound (100 mg, 79% yield) as yellow solid; LCMS (ESI, M+1): m/z=553.5.

Step F. (1R,5R,6R)-3-(7-(8-ethyl-7-fluoro-3-(methoxymethoxy)naphthalen-1-yl)-2-(((Z)-2-(fluoromethylene)tetrahydro-1H-pyrrolizin-7a(5H)-yl)methoxy)-5,6,7,8-tetrahydropyrido[3,4-d]pyrimidin-4-yl)-3-azabicyclo[3.2.1]octan-6-ol: To a mixture of (Z)-7-(8-ethyl-7-fluoro-3-(methoxymethoxy)naphthalen-1-yl)-2-((2-(fluoromethylene)tetrahydro-1H-pyrrolizin-7a(5H)-yl)methoxy)-4-methoxy-5,6,7,8-tetrahydropyrido[3,4-d]pyrimidine (50.0 mg, 1.0 equiv), PYBOP (70.6 mg, 1.5 equiv) and TEA (27.5 mg, 3.0 equiv) in DMSO (0.50 mL) was added (1R,5R,6R)-3-azabicyclo[3.2.1]octan-6-ol (17.3 mg, 1.5 equiv). The reaction was stirred at 20° C. for 1 hour. The mixture was filtered and purified by reversed phase flash chromatography [C18, 0.1% formic acid condition] to afford the title compound (40.0 mg, 60% yield) as yellow solid; LCMS (ESI, M+1): m/z=662.4.

Step G. (1R,5R,6R)-3-(7-(8-ethyl-7-fluoro-3-hydroxynaphthalen-1-yl)-2-(((Z)-2-(fluoromethylene)tetrahydro-1H-pyrrolizin-7a(5H)-yl)methoxy)-5,6,7,8-tetrahydropyrido[3,4-d] pyrimidin-4-yl)-3-azabicyclo[3.2.1]octan-6-ol: A mixture of (1R,5R,6R)-3-(7-(8-ethyl-7-fluoro-3-(methoxymethoxy)naphthalen-1-yl)-2-(((Z)-2-(fluoromethylene)tetrahydro-1H-pyrrolizin-7a(5H)-yl)methoxy)-5,6,7,8-tetrahydropyrido[3,4-d]pyrimidin-4-yl)-3-azabicyclo[3.2.1]octan-6-ol (35.0 mg 1.0 equiv) and HCl•MeOH (4 M, 0.5 mL, 38 equiv) was stirred at 20° C. for 0.5 hours. The mixture was concentrated, diluted with saturated NaHCO₃ aqueous solution (1.0 mL) and extracted with Ethyl acetate (3×2.0 mL). The combined organic layers were washed with brine (3×2.0 mL), dried over anhydrous sodium sulfate, concentrated and purified with prep-HPLC [Phenomenex luna C18 150×25 mm×10 μm; A: water (0.1% FA), B: ACN, B %: 19%-49% over 10 min] to afford the title compound (6.14 mg, 18% yield, 0.19 HCOOH) as white solid; ¹H NMR (400 MHz, METHANOL-d₄) δ=7.60-7.41 (m, 1H), 7.16 (br t, J=9.4 Hz, 1H), 7.07-6.91 (m, 2H), 6.84-6.50 (m, 1H), 4.68-4.52 (m, 1H), 4.42-4.03 (m, 5H), 3.93-3.78 (m, 1H), 3.71-3.53 (m, 2H), 3.53-3.37 (m, 4H), 3.29-3.14 (m, 3H), 3.03 (br dd, J=6.9, 19.3 Hz, 1H), 2.75 (br d, J=15.6 Hz, 3H), 2.47 (br d, J=15.8 Hz, 1H), 2.32-2.11 (m, 4H), 2.06-1.85 (m, 3H), 1.81-1.69 (m, 2H), 1.32 (br d, J=5.9 Hz, 1H), 1.17-1.09 (m, 3H); LCMS (ESI, M+1): m/z=618.5.

### Example 577

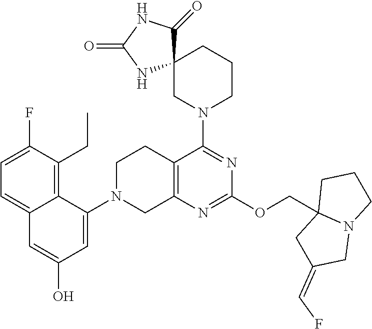 [SMILES: CCc1c(F)ccc2cc(O)cc(N3CCc4c(nc(OCC56CCCN5C/C(=C\F)C6)nc4N4CCC[C@]5(C4)NC(=O)NC5=O)C3)c12]

### (5R)-7-(7-(8-ethyl-7-fluoro-3-hydroxynaphthalen-1-yl)-2-(((Z)-2-(fluoromethylene)tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione

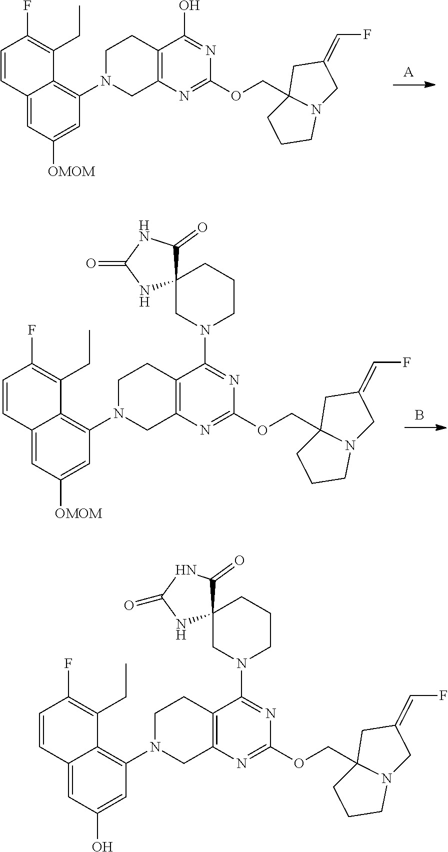

Step A. (5R)-7-(7-(8-ethyl-7-fluoro-3-(methoxymethoxy)naphthalen-1-yl)-2-(((Z)-2-(fluoromethylene)tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione: To a solution of (Z)-7-(8-ethyl-7-fluoro-3-(methoxymethoxy)naphthalen-1-yl)-2-((2-(fluoromethylene)tetrahydro-1H-pyrrolizin-7a(5H)-yl)methoxy)-5,6,7,8-tetrahydropyrido[3,4-d]pyrimidin-4-ol (30.0 mg, 1.0 equiv), PYBOP (42.4 mg, 1.5 equiv) and TEA (16.5 mg, 3.0 equiv) in DMSO (0.10 mL) was added (R)-1,3,7-triazaspiro[4.5]decane-2,4-dione (13.8 mg, 1.5 equiv). The reaction was stirred at 40° C. for 3 hours. The mixture was filtered and purified by reversed phase flash chromatography [C18, 0.1% formic acid condition] to afford the title compound (25.0 mg, 65% yield) as yellow solid; LCMS (ESI, M+1): m/z=704.5.

Step B. (5R)-7-(7-(8-ethyl-7-fluoro-3-hydroxynaphthalen-1-yl)-2-(((Z)-2-(fluoromethylene)tetrahydro-1H-pyrrolizin-7a(5H)-yl)methoxy)-5,6,7,8-tetrahydropyrido[3,4-d] pyrimidin-4-yl)-1,3,7-triazaspiro[4.5]decane-2,4-dione: A solution of (5R)-7-(7-(8-ethyl-7-fluoro-3-(methoxymethoxy)naphthalen-1-yl)-2-(((Z)-2-(fluoromethylene)tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (20.0 mg, 1.0 equiv) in HCl•MeOH (4 M, 7.10 μL, 1.0 equiv) was stirred at 20° C. for 10 minutes. The mixture was concentrated, diluted with saturated NaHCO₃ aqueous solution (1.0 mL) and extracted with Ethyl acetate (3×2.0 mL). The combined organic layers were washed with brine (3×2.0 mL), dried over anhydrous sodium sulfate, concentrated and purified by prep-HPLC [Phenomenex luna C18 150×25 mm×10 μm; A: water (0.1% FA), B: ACN, B %: 28%-48% over 10 min] to afford the title compound (4.38 mg, 22% yield, 0.52 HCOOH) as yellow solid; ¹H NMR (400 MHz, METHANOL-d₄) δ=7.60-7.46 (m, 1H), 7.14 (br t, J=9.2 Hz, 1H), 7.05-6.89 (m, 2H), 6.83-6.52 (m, 1H), 4.31-4.08 (m, 4H), 4.07-3.85 (m, 2H), 3.71-3.55 (m, 2H), 3.49 (br d, J=13.6 Hz, 1H), 3.45-3.36 (m, 3H), 3.24-3.12 (m, 3H), 3.10-2.98 (m, 1H), 2.88-2.68 (m, 3H), 2.55-2.41 (m, 1H), 2.21-2.07 (m, 2H), 2.04-1.79 (m, 6H), 1.17-1.03 (m, 3H); LCMS (ESI, M+1): m/z=660.5.

### Example 578

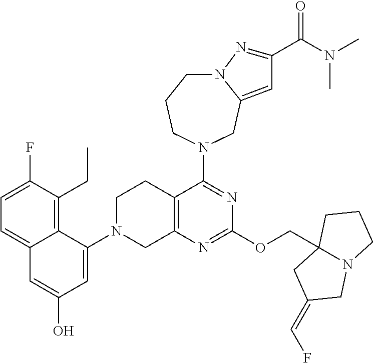 [SMILES: CCc1c(F)ccc2cc(O)cc(N3CCc4c(nc(OCC56CCCN5C/C(=C\F)C6)nc4N4CCCn5nc(C(=O)N(C)C)cc5C4)C3)c12]

### (Z)-5-(7-(8-ethyl-7-fluoro-3-hydroxynaphthalen-1-yl)-2-((2-(fluoromethylene)tetrahydro-1H-pyrrolizin-7a(5H)-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide

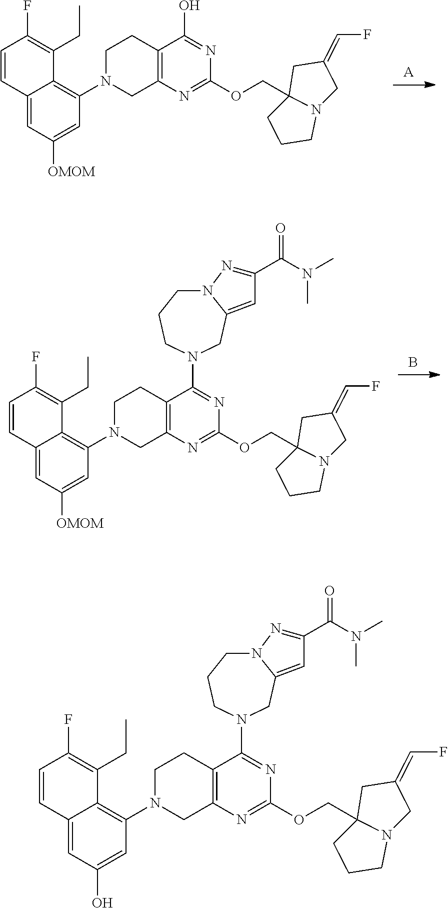

Synthesized according to Example 577. The title compound was obtained as white solid (0.2 eq HCOOH). H NMR (400 MHz, METHANOL-d₄) δ=7.55-7.47 (m, 1H), 7.14 (br t, J=9.2 Hz, 1H), 6.97 (s, 2H), 6.79-6.51 (m, 2H), 4.98 (br s, 1H), 4.60-4.52 (m, 4H), 4.23-4.00 (m, 5H), 3.93-3.84 (m, 1H), 3.67 (br d, J=17.2 Hz, 1H), 3.58-3.48 (m, 2H), 3.38 (br d, J=6.8 Hz, 2H), 3.25-3.16 (m, 3H), 3.08 (s, 3H), 2.79-2.63 (m, 3H), 2.50-2.24 (m, 2H), 2.12-1.82 (m, 6H), 1.38-1.25 (m, 1H), 1.11 (br t, J=7.2 Hz, 3H); LCMS (ESI, M+1). m/z=699.6.

### Example 579

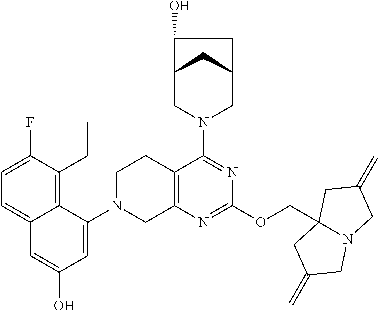 [SMILES: C=C1CN2CC(=C)CC2(COc2nc3c(c(N4C[C@@H]5C[C@H](C4)[C@H](O)C5)n2)CCN(c2cc(O)cc4ccc(F)c(CC)c24)C3)C1]

### (1R,5R,6R)-3-(2-((2,6-dimethylenetetrahydro-1H-pyrrolizin-7a(5H)-yl)methoxy)-7-(8-ethyl-7-fluoro-3-hydroxynaphthalen-1-yl)-5,6,7,8-tetrahydropyrido[3,4-d]pyrimidin-4-yl)-3-azabicyclo[3.2.1]octan-6-ol

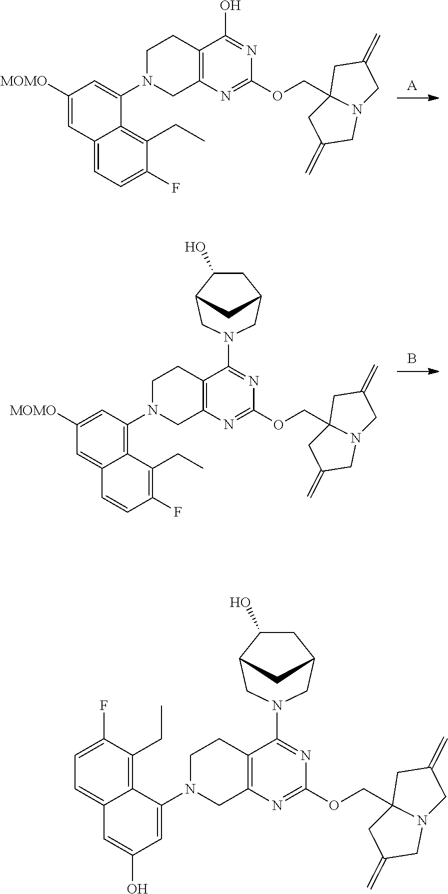

Synthesized according to Example 582. The title compound was as white solid; ¹H NMR (400 MHz, METHANOL-d₄) δ=7.50 (ddd, J=1.2, 6.0, 8.8 Hz, 1H), 7.18-7.10 (m, 1H), 7.02-6.93 (m, 2H), 5.00 (br dd, J=2.0, 4.8 Hz, 4H), 4.65-4.44 (m, 1H), 4.36-4.01 (m, 5H), 3.84-3.69 (m, 3H), 3.63 (dd, J=3.6, 17.6 Hz, 1H), 3.47-3.37 (m, 3H), 3.26-2.92 (m, 4H), 2.82-2.65 (m, 3H), 2.58-2.49 (m, 2H), 2.30-2.13 (m, 3H), 1.83-1.51 (m, 3H), 1.38-1.24 (m, 1H), 1.12 (dt, J=4.8, 7.2 Hz, 3H); LCMS (ESI, M+1): m/z=612.4.

### Example 580

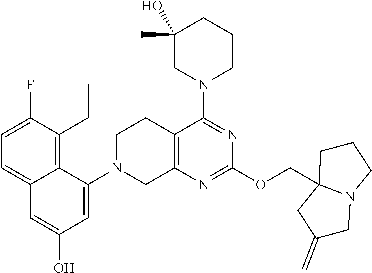 [SMILES: C=C1CN2CCCC2(COc2nc3c(c(N4CCC[C@@](C)(O)C4)n2)CCN(c2cc(O)cc4ccc(F)c(CC)c24)C3)C1]

### (3R)-1-(7-(8-ethyl-7-fluoro-3-hydroxynaphthalen-1-yl)-2-((2-methylenetetrahydro-1H-pyrrolizin-7a(5H)-yl)methoxy)-5,6,7,8-tetrahydropyrido[3,4-d]pyrimidin-4-yl)-3-methylpiperidin-3-ol

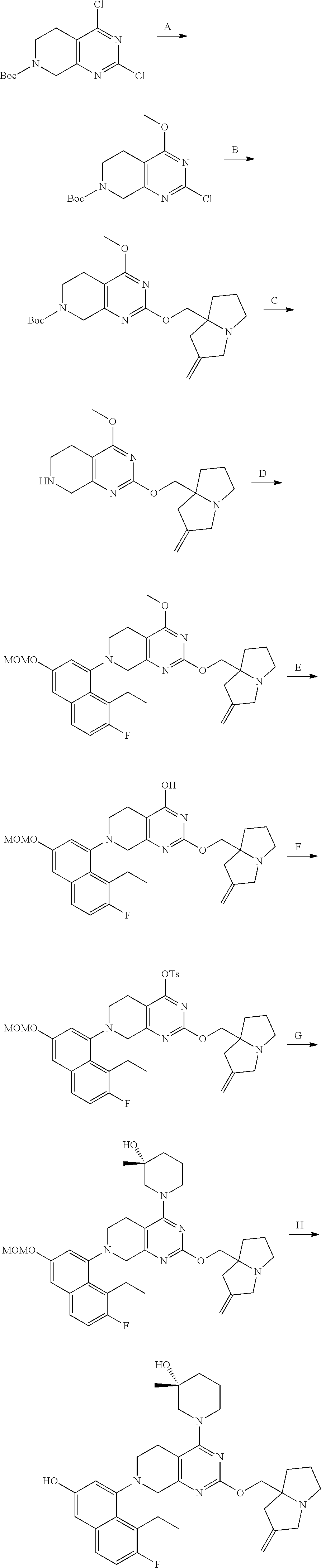

Step A. tert-butyl 2-chloro-4-methoxy-5,8-dihydropyrido[3,4-d]pyrimidine-7(6H)-carboxylate: To a solution of tert-butyl 2,4-dichloro-6,8-dihydro-5H-pyrido[3,4-d]pyrimidine-7-carboxylate (18.0 g, 1.0 equiv) in MeOH (100 mL) was added NaOMe (21.3 g, 30% purity, 2.0 equiv). The reaction was stirred at 0° C. for 2 hours. The mixture was diluted with water (100 mL) and extracted with ethyl acetate (2×120 mL). The combined organic layers were washed with brine (100 mL), dried over anhydrous sodium sulfate, concentrated and purified with column chromatography [SiO2, petroleum ether/ethyl acetate=0/1 to 10/1] to afford the title compound (13.7 g, 75% yield) as white solid.

Step B. tert-butyl 4-methoxy-2-((2-methylenetetrahydro-1H-pyrrolizin-7a(5H)-yl)methoxy)-5,8-dihydropyrido[3,4-d]pyrimidine-7(6H)-carboxylate: To a solution of (6-methylene-2,3,5,7-tetrahydro-1H-pyrrolizin-8-yl)methanol (3.10 g, 1.0 equiv) and tert-butyl 2-chloro-4-methoxy-6,8-dihydro-5H-pyrido[3,4-d]pyrimidine-7-carboxylate (6.00 g, 1.0 equiv) in toluene (25 mL) were added Pd(OAc)₂ (449 mg, 0.1 equiv), BINAP (2.50 g, 0.2 equiv) and Cs₂CO₃ (20.0 g, 3.0 equiv). The reaction was degassed and purged with N₂ for 3 times. The reaction was stirred at 110° C. for 0.5 hours under N₂ atmosphere. The mixture was diluted with water (50 mL) and extracted with ethyl acetate (2×50 mL). The combined organic layers were washed with brine (100 mL), dried over anhydrous sodium sulfate, concentrated and purified with prep-HPLC [Phenomenex luna C18 (250×70 mm, 10 μm); mobile phase: water (FA)-ACN; gradient: 10%-35% B over 25 min] to afford the title compound (226 mg, 76% yield) as yellow solid; LCMS (ESI, M+1): m/z=417.2.

Step C. 4-methoxy-2-((2-methylenetetrahydro-1H-pyrrolizin-7a(5H)-yl)methoxy)-5,6,7,8-tetrahydropyrido[3,4-d]pyrimidine: To a solution of tert-butyl 4-methoxy-2-((2-methylenetetrahydro-1H-pyrrolizin-7a(5H)-yl)methoxy)-5,8-dihydropyrido[3,4-d]pyrimidine-7(6H)-carboxylate(2.2 g, 1.0 equiv) in MeOH (8 mL) was added HCl-MeOH (8 mL). The reaction was stirred at 25° C. for 3 hours. The reaction was concentrated, diluted with DCM/MeOH=10/1 (60 mL) and washed with sodium hydroxide solution (1 N, 4×80 mL). The combined organic layers were washed with brine (40 mL), dried over anhydrous sodium sulfate, concentrated to afford the title compound (1.35 g, 81% yield) as yellow oil; LCMS (ESI, M+1): m/z=317.1.

Step D. 7-(8-ethyl-7-fluoro-3-(methoxymethoxy)naphthalen-1-yl)-4-methoxy-2-((2-methylenetetrahydro-1H-pyrrolizin-7a(5H)-yl)methoxy)-5,6,7,8-tetrahydropyrido[3,4-d]pyrimidine: To a solution of 4-methoxy-2-((2-methylenetetrahydro-1H-pyrrolizin-7a(5H)-yl)methoxy)-5,6,7,8-tetrahydropyrido[3,4-d]pyrimidine(1.25 g, 1.0 equiv) and [8-ethyl-7-fluoro-3-(methoxymethoxy)-1-naphthyl]trifluoromethanesulfonate (1.51 g, 1.0 equiv) in toluene (11.0 mL) were added Pd₂(dba)₃ (362 mg, 0.1 equiv), Xantphos (457 mg, 0.2 equiv) and Cs₂CO₃ (3.86 g, 3.0 equiv). The reaction was degassed and purged with N₂ for 3 times. The reaction was stirred at 110° C. for 12 hours under N₂ atmosphere. The mixture was diluted with water (50 mL) and extracted with ethyl acetate (2×30 mL). The combined organic layers were washed with brine (30 mL), dried over anhydrous sodium sulfate, concentrated and purified with prep-HPLC [column: Phenomenex luna C18 150×40 mm×15 μm; mobile phase: water(FA)-ACN; gradient: 35%-⁶⁵% B over 15 min; gradient: 30%-⁶⁰% B over min] to afford the title compound (178 mg, 8.1% yield) as yellow oil; LCMS (ESI, M+1): m/z=549.3.

Step E. 7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d] pyrimidin-4-ol: To a solution of 7-(8-ethyl-7-fluoro-3-(methoxymethoxy)naphthalen-1-yl)-4-methoxy-2-((2-methylenetetrahydro-1H-pyrrolizin-7a(5H)-yl)methoxy)-5,6,7,8-tetrahydropyrido[3,4-d]pyrimidine (175 mg, 1.0 equiv) in DMAc (2.0 mL) was added NaSEt (134 mg, 5.0 equiv). The reaction was stirred at 60° C. for 1.5 hours. The mixture was diluted with water (10 mL) and extracted with ethyl acetate (3×20 mL). The combined organic layers were washed with brine (10 mL), dried over anhydrous sodium sulfate, concentrated and purified with prep-HPLC [column: YMC Triart C18 150×25 mm×5 μm; mobile phase: water(FA)-ACN; gradient: 20%-50% B over 10 min] to afford the title compound (76 mg, 38% yield) as yellow solid; LCMS (ESI, M+1): m/z=535.3.

Step F. 7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d]pyrimidin-4-yl 4-methylbenzenesulfonate: To a solution of 7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d]pyrimidin-4-ol (70.0 mg, 1.0 equiv) and TEA (39.7 mg, 3.0 equiv) in DCM (1.5 mL) were added 4-methylbenzenesulfonyl chloride (37.4 mg, 1.5 equiv) and DMAP (3.20 mg, 0.2 equiv) at 0° C. The reaction was stirred at 25° C. for 4 hours. The mixture was diluted with water (10 mL) and extracted with ethyl acetate (2×10 mL). The combined organic layers were washed with brine (10 mL), dried over anhydrous sodium sulfate, concentrated and purified with column chromatography [SiO2, petroleum ether/ethyl acetate=5/1 to 0/1] to afford the title compound (45.0 mg, 55% yield) as yellow oil; LCMS (ESI, M+1): m/z=689.4.

Step G. (3R)-1-(7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d]pyrimidin-4-yl)-3-methylpiperidin-3-ol: To a solution of 7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d]pyrimidin-4-yl 4-methylbenzenesulfonate (30.0 mg, 1.0 equiv) and DIEA (28.1 mg, 5.0 equiv) in DMF (1 mL) were added (3R)-3-methylpiperidin-3-ol (6.60 mg, 1.0 equiv, HCl) and 4 Å molecular sieves (10.0 mg). The reaction was stirred at 80° C. for 1 hour. The mixture was diluted with water (10 mL) and extracted with ethyl acetate (2×10 mL). The combined organic layers were washed with brine (10 mL), dried over anhydrous sodium sulfate, concentrated to afford the title compound (20 mg, crude) as yellow oil; LCMS (ESI, M+1): m/z=632.3.

Step H. (3R)-1-(7-(8-ethyl-7-fluoro-3-hydroxynaphthalen-1-yl)-2-((2-methylenetetrahydro-1H-pyrrolizin-7a(5H)-yl)methoxy)-5,6,7,8-tetrahydropyrido[3,4-d] pyrimidin-4-yl)-3-methylpiperidin-3-ol: To a solution of (3R)-1-(7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d]pyrimidin-4-yl)-3-methylpiperidin-3-ol (20 mg, 1.0 equiv) in MeOH (0.5 mL) was added HCl•MeOH (0.5 mL). The reaction was stirred at 0° C. for 1 hour. The mixture was concentrated, dissolved in MeOH (5 mL), adjusted to pH=7 with solid NaHCO₃ and filtered. The filtration was concentrated and purified with prep-HPLC [column: Phenomenex luna C18 150×25 mm×10 μm; mobile phase: water(FA)-ACN; gradient: 18%-48% B over 10 min] to afford the title compound (4.42 mg, 24% yield) as off-white solid; ¹H NMR (400 MHz, METHANOL-d₄) δ=8.54 (s, 1H), 7.56-7.46 (m, 1H), 7.14 (t, J=9.2 Hz, 1H), 7.01-6.93 (m, 2H), 5.08 (br s, 2H), 4.34-4.20 (m, 2H), 4.08 (dd, J=4.0, 17.6 Hz, 1H), 3.92 (br d, J=13.2 Hz, 1H), 3.73-3.61 (m, 2H), 3.55-3.35 (m, 6H), 3.25-3.08 (m, 3H), 2.91-2.68 (m, 3H), 2.55 (br d, J=16.0 Hz, 1H), 2.31-1.50 (m, 9H), 1.28-1.19 (m, 3H), 1.12 (t, J=7.2 Hz, 3H); LCMS (ESI, M+1): m/z=588.3.

### Example 581

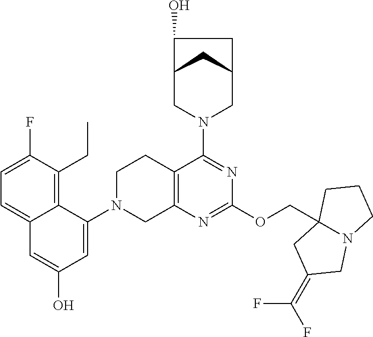 [SMILES: CCc1c(F)ccc2cc(O)cc(N3CCc4c(nc(OCC56CCCN5CC(=C(F)F)C6)nc4N4C[C@@H]5C[C@H](C4)[C@H](O)C5)C3)c12]

### (1R,5R,6R)-3-(2-((2-(difluoromethylene)tetrahydro-1H-pyrrolizin-7a(5H)-yl)methoxy)-7-(8-ethyl-7-fluoro-3-hydroxynaphthalen-1-yl)-5,6,7,8-tetrahydropyrido[3,4-d]pyrimidin-4-yl)-3-azabicyclo[3.2.1]octan-6-ol

Synthesized according to Example 575. The title compound was obtained as white solid; ¹H NMR (400 MHz, METHANOL-d₄) δ=7.54-7.47 (m, 1H), 7.17-7.10 (m, 1H), 7.00-6.97 (m, 1H), 6.97-6.94 (m, 1H), 4.67-4.43 (m, 1H), 4.39-3.96 (m, 5H), 3.85-3.53 (m, 3H), 3.40 (br d, J=13.2 Hz, 4H), 3.14-2.97 (m, 3H), 2.80-2.62 (m, 3H), 2.45 (br d, J=15.2 Hz, 1H), 2.30-2.14 (m, 3H), 2.11-1.85 (m, 4H), 1.82-1.53 (m, 3H), 1.33-1.28 (m, 1H), 1.12 (dt, J=4.4, 7.1 Hz, 3H); LCMS (ESI, M+1): m/z=636.4.

### Example 582

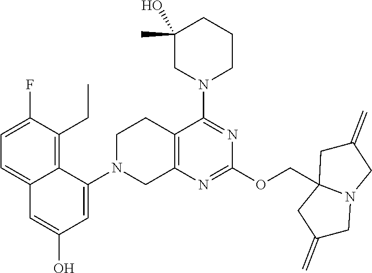 [SMILES: C=C1CN2CC(=C)CC2(COc2nc3c(c(N4CCC[C@@](C)(O)C4)n2)CCN(c2cc(O)cc4ccc(F)c(CC)c24)C3)C1]

### (R)-1-(2-((2,6-dimethylenetetrahydro-1H-pyrrolizin-7a(5H)-yl)methoxy)-7-(8-ethyl-7-fluoro-3-hydroxynaphthalen-1-yl)-5,6,7,8-tetrahydropyrido[3,4-d]pyrimidin-4-yl)-3-methylpiperidin-3-ol

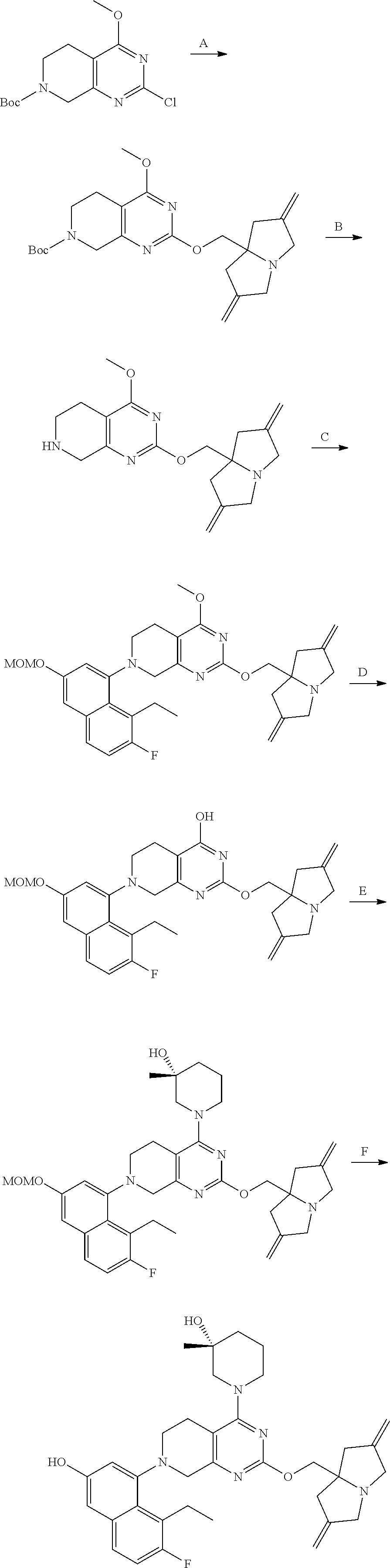

Step A. tert-butyl 2-((2,6-dimethylenetetrahydro-1H-pyrrolizin-7a(5H)-yl)methoxy)-4-methoxy-5,8-dihydropyrido[3,4-d] pyrimidine-7(6H)-carboxylate: To a solution of tert-butyl 2-chloro-4-methoxy-5,8-dihydropyrido[3,4-d]pyrimidine-7(6H)-carboxylate (4.00 g, 1.0 equiv) and (2,6-dimethylenetetrahydro-1H-pyrrolizin-7a(5H)-yl)methanol (2.65 g, 1.2 equiv) in DMF (20 mL) and THE (20 mL) were added DABCO (1.20 g, 0.8 equiv) and Cs₂CO₃ (13.0 g, 3.0 equiv). The reaction was stirred at 40° C. for 10 hours. The mixture was diluted with water (100 mL) and extracted with ethyl acetate (2×50 mL). The combined organic layers were washed with brine (100 mL), dried over anhydrous sodium sulfate, concentrated and purified with column chromatography [SiO₂, petroleum ether/ethyl acetate=I/O to 3/1] to afford the title compound (2.40 g, 37% yield) as yellow solid; LCMS (ESI, M+1): m/z=429.3.

Step B. 2-((2,6-dimethylenetetrahydro-1H-pyrrolizin-7a(5H)-yl)methoxy)-4-methoxy-5,6,7,8-tetrahydropyrido[3,4-d] pyrimidine: To a solution of tert-butyl 2-((2,6-dimethylenetetrahydro-1H-pyrrolizin-7a(5H)-yl)methoxy)-4-methoxy-5,8-dihydropyrido[3,4-d]pyrimidine-7(6H)-carboxylate (2.40 g, 1.0 equiv) in MeOH (10 mL) was added HCl-MeOH (10 mL). The reaction was stirred at 25° C. for 1 hour. The mixture was diluted with NaOH aqueous (1 N, 80 mL) and extracted with DCM/MeOH=10/1 (2×50 mL). The combined organic layers were washed with brine (100 mL), dried over anhydrous sodium sulfate and concentrated to afford the title compound (1.70 g, 91% yield) as yellow solid; LCMS (ESI, M+1): m/z=329.1.

Step C. 2-((2,6-dimethylenetetrahydro-1H-pyrrolizin-7a(5H)-yl)methoxy)-7-(8-ethyl-7-fluoro-3-(methoxymethoxy)naphthalen-1-yl)-4-methoxy-5,6,7,8-tetrahydropyrido[3,4-d] pyrimidine: To a solution of 2-((2,6-dimethylenetetrahydro-1H-pyrrolizin-7a(5H)-yl)methoxy)-4-methoxy-5,6,7,8-tetrahydropyrido[3,4-d]pyrimidine (1.70 g, 1.0 equiv) and 8-ethyl-7-fluoro-3-(methoxymethoxy)naphthalen-1-yl trifluoromethanesulfonate (2.97 g, 1.5 equiv) in toluene (20 mL) were added Pd₂(dba)₃ (474 mg, 0.1 equiv), Xantphos (599 mg, 0.2 equiv) and Cs₂CO₃ (5.06 g, 3 equiv). The reaction was degassed and purged with N₂ for 3 times and stirred at 90° C. for 12 hours under N₂ atmosphere. The mixture was diluted with water (100 mL) and extracted with ethyl acetate (2×50 mL). The combined organic layers were washed with brine (100 mL), dried over anhydrous sodium sulfate, concentrated and purified with reversed-phase HPLC [0.1% FA condition] to afford the title compound (145 mg, 5% yield) as brown solid; LCMS (ESI, M+1): m/z=561.3.

Step D. 2-((2,6-dimethylenetetrahydro-1H-pyrrolizin-7a(5H)-yl)methoxy)-7-(8-ethyl-7-fluoro-3-(methoxymethoxy)naphthalen-1-yll-5,6,7,8-tetrahydropyrido[3,4-d]pyrimidin-4-ol: To a solution of 2-((2,6-dimethylenetetrahydro-1H-pyrrolizin-7a(5H)-yl)methoxy)-7-(8-ethyl-7-fluoro-3-(methoxymethoxy)naphthalen-1-yl)-4-methoxy-5,6,7,8-tetrahydropyrido[3,4-d]pyrimidine (130 mg, 1.0 equiv) in DMAc (3 mL) was added NaSEt (97.5 mg, 5.0 equiv). The reaction was stirred at 60° C. for 1 hour. The mixture was diluted with water (30 mL) and extracted with DCM (2×30 mL). The combined organic layers were washed with brine (30 mL), dried over anhydrous sodium sulfate, concentrated and purified with prep-TLC [SiO₂, DCM/MeOH=10/1] to afford the title compound (80.0 mg, 58% yield) as brown solid; LCMS (ESI, M+1): m/z=547.4.

Step E. (R)-i-(2-(2,6-dimethylenetetrahydro-1H-pyrrolizin-7a(5H)-ylimethoxy)-7-(8-ethyl-7-fluoro-3-(methoxymethoxy)naphthalen-1-yl)-5,6,78-tetrahydropyrido[3,4-d]pyrimidin-4-yl)-3-methylpiperidin-3-ol: To a solution of 2-((2,6-dimethylenetetrahydro-1H-pyrrolizin-7a(5H)-yl)methoxy)-7-(8-ethyl-7-fluoro-3-(methoxymethoxy)naphthalen-1-yl)-5,6,7,8-tetrahydropyrido[3,4-d]pyrimidin-4-ol (35.0 mg, 1 equiv) in DMSO (0.4 mL) were added TEA (32.4 mg, 5.0 equiv) and PyBOP (50.0 mg, 1.5 equiv). The reaction was stirred at 25° C. for 20 minutes and (R)-3-methylpiperidin-3-ol (14.6 mg, 1.5 equiv, HCl) was added. The reaction was stirred at 25° C. for 1 hour. The mixture was diluted with water (10 mL) and extracted with ethyl acetate (2×10 mL). The combined organic layers were washed with brine (10 mL), dried over anhydrous sodium sulfate, concentrated and purified with prep-TLC [SiO₂, DCM/MeOH=10/1] to afford the title compound (42.0 mg, 98% yield) as yellow solid; LCMS (ESI, M+1): m/z=644.4.

Step F. (R)-1-(2-((2,6-dimethylenetetrahydro-1H-pyrrolizin-7a(5H)-yl)methoxy)-7-(8-ethyl-7-fluoro-3-hydroxynaphthalen-1-yl)-5,6,7,8-tetrahydropyrido[3,4-d]pyrimidin-4-yl)-3-methylpiperidin-3-ol: To a solution of (R)-1-(2-((2,6-dimethylenetetrahydro-1H-pyrrolizin-7a(5H)-yl)methoxy)-7-(8-ethyl-7-fluoro-3-(methoxymethoxy)naphthalen-1-yl)-5,6,7,8-tetrahydropyrido[3,4-d]pyrimidin-4-yl)-3-methylpiperidin-3-ol (40.0 mg, 1.0 equiv) in MeOH (2 mL) was added HCl•MeOH (2 mL). The reaction was stirred at 0° C. for 2 hours. The mixture was concentrated, dissolved in MeOH (2 mL), adjusted to pH=7 with solid NaHCO₃ and filtered. The filtration was concentrated and purified with prep-HPLC [column: Waters Xbridge 150×25 mm×5 μm; mobile phase: water(NH₄HCO₃)-ACN; gradient: 56%-86% B over 9 minutes] to afford the title compound (11.9 mg, 31% yield) as white solid; ¹H NMR (400 MHz, METHANOL-d₄) δ=7.51 (ddd, J=1.2, 6.0, 8.8 Hz, 1H), 7.14 (t, J=9.2 Hz, 1H), 6.99 (dd, J=2.4, 4.0 Hz, 1H), 6.97-6.94 (m, 1H), 5.00 (br d, J=4.4 Hz, 4H), 4.22-4.13 (m, 2H), 4.08 (dd, J=4.0, 18.0 Hz, 1H), 3.94-3.55 (m, 5H), 3.54-3.33 (m, 6H), 3.30-3.28 (m, 1H), 3.19-3.12 (m, 2H), 2.84-2.63 (m, 3H), 2.54 (br d, J=16.0 Hz, 2H), 2.16-1.80 (m, 1H), 1.79-1.56 (m, 3H), 1.28-1.18 (m, 3H), 1.12 (t, J=7.2 Hz, 3H); LCMS (ESI, M+1): m/z=600.4.

### Example 583

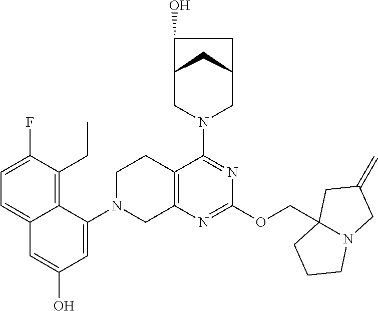 [SMILES: C=C1CN2CCCC2(COc2nc3c(c(N4C[C@@H]5C[C@H](C4)[C@H](O)C5)n2)CCN(c2cc(O)cc4ccc(F)c(CC)c24)C3)C1]

### (1R,5R,6R)-3-(7-(8-ethyl-7-fluoro-3-hydroxynaphthalen-1-yl)-2-((2-methylenetetrahydro-1H-pyrrolizin-7a(5H)-yl)methoxy)-5,6,7,8-tetrahydropyrido[3,4-d]pyrimidin-4-yl)-3-azabicyclo[3.2.1]octan-6-ol

Synthesized according to Example 580. The title compound was obtained as off-white solid (FA salt). ¹H NMR (400 MHz, METHANOL-d₄) δ=8.55 (s, 1H), 7.53-7.48 (m, 1H), 7.16-7.11 (m, 1H), 7.04-6.88 (m, 2H), 5.06 (br s, 2H), 4.65-4.50 (m, 1H), 4.35-4.16 (m, 4H), 4.07 (br dd, J=4.8, 18.0 Hz, 1H), 3.95-3.76 (m, 2H), 3.63 (br d, J=18.0 Hz, 1H), 3.51-3.35 (m, 5H), 3.15-2.95 (m, 3H), 2.89-2.68 (m, 3H), 2.52 (br d, J=15.6 Hz, 1H), 2.29-2.15 (m, 4H), 2.04-1.85 (m, 3H), 1.79-1.69 (m, 2H), 1.59-1.28 (m, 1H), 1.14-1.09 (m, 3H); LCMS (ESI, M+1): m/z=600.5.

### Example 584

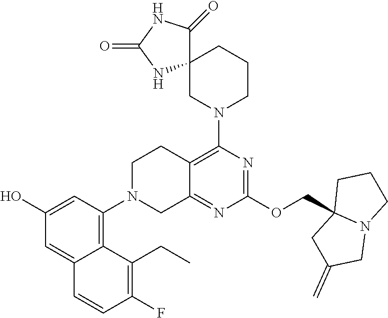 [SMILES: C=C1CN2CCC[C@@]2(COc2nc3c(c(N4CCC[C@]5(C4)NC(=O)NC5=O)n2)CCN(c2cc(O)cc4ccc(F)c(CC)c24)C3)C1]

### (R)-7-(7-(8-ethyl-7-fluoro-3-hydroxynaphthalen-1-yl)-2-(((S)-2-methylenetetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione

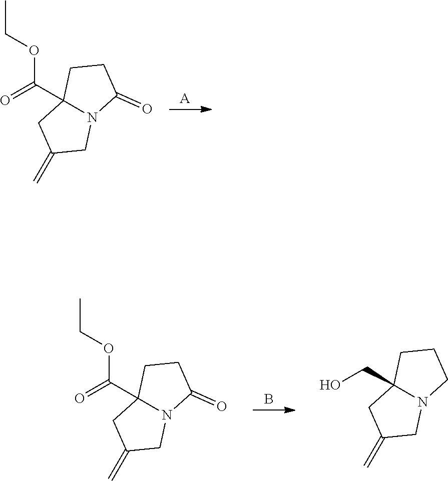

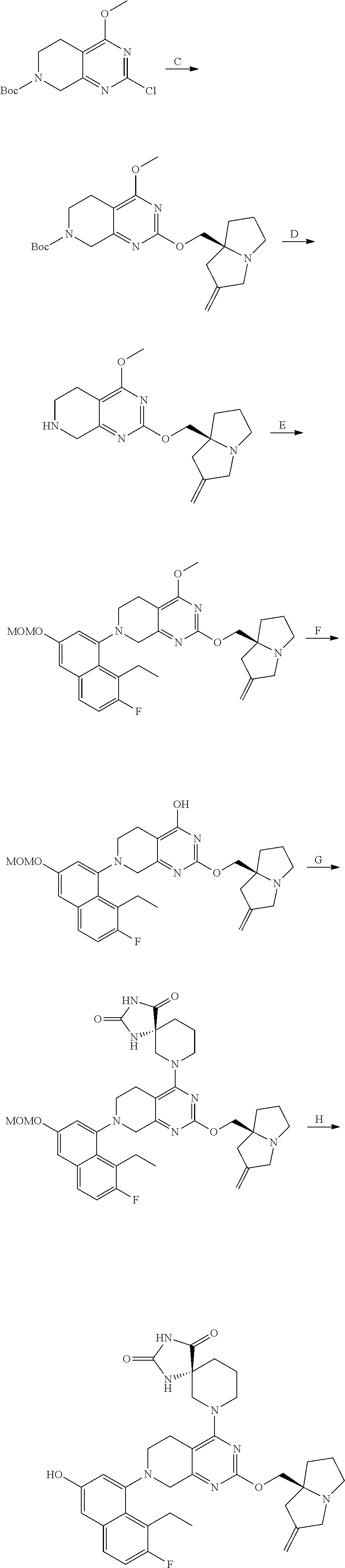

Step B. et (S)-2-methylene -5-xtetrahydro-IH-pyrrolizine-7a(5H)-ylmtao:T saroltn ofraei ethyl ( )₂-methylene-5-oxotetrahydro-H-pyrrolizine-7a(5H)-croyae(0.7g 1.0oylt equiv ineTHF(70tmL wash adde DIBl-Hn (1CE M,H323LA 0, equiv drpws atmm 0° mde N₂.tio The retion waS)stirredty0e° Cforxours.hermixtuyrerwaszquenchdcawithywate (2.97 mL, 15% NaOH (12.9 mL), and water (32.4 mL) under N₂ at 0° C. The mixture was stirred at room temperature for 15 minutes and filtered. The filtrate was dried over anhydrous sodium sulfate, concentrated and purified with chromatography (Al₂O₃, petroleum ether/ethyl acetate 1/1 to ethyl acetate/methanol 20/1) to afford the title compound (2.83 g, 51% yield) as yellow oil; ¹H NMR (400 MHz, CHLOROFORM-dl) 6 4.94-4.84 (m, 2H), 3.62 (br d, J=14.8 Hz, 1H), 3.32-3.20 (m, 3H), 3.11-3.03 (m, 1H), 3.03-2.83 (m, 1H), 2.70-2.60 (m, 1H), 2.50-2.40 (m, 1H), 2.36-2.28 (m, 1H), 1.95-1.83 (m, 2H), 1.82-1.73 (m, 2H).

Step C. tert-butyl (S)-4-methoxy-2-((2-methylenetetrahydro-1H-pyrrolizin-7a(5H)-yl)methoxy)-5,8-dihydropyrido[3,4-d]pyrimidine-7(6H)-carboxylate: To a mixture of tert-butyl 2-chloro-4-methoxy-5,8-dihydropyrido[3,4-d]pyrimidine-7(6H)-carboxylate (5 g, 1 equiv) and (S)-(2-methylenetetrahydro-1H-pyrrolizin-7a(5H)-yl)methanol (2.56 g, 1 equiv) in toluene (75 mL) were added Pd(OAc)₂ (374 mg, 0.1 equiv), BINAP (1.04 g, 0.1 equiv) and Cs₂CO₃ (13.6 g, 2.5 equiv). The reaction was degassed and purged with nitrogen 3 times. The reaction was stirred at 110° C. for 2 hours. The mixture was filtered, concentrated, and purified with silica gel chromatography (petroleum ether/ethyl acetate 3/1 to 1/1) followed by reversed phase flash chromatography [C18, 0.1% formic acid condition] to afford the title compound (4.5 g, 61% yield) as yellow oil; LCMS (ESI, M+1): m/z=417.2.

Step D. (S)-4-methoxy-2-((2-methylenetetrahydro-1H-pyrrolizin-7a(5H)-yll)methoxy)-5,6,7,8-tetrahydropyrido[3,4-d]pyrimidine: To a solution of tert-butyl (S)-4-methoxy-2-((2-methylenetetrahydro-1H-pyrrolizin-7a(5H)-yl)methoxy)-5,8-dihydropyrido[3,4-d]pyrimidine-7(6H)-carboxylate (5.85 g, 1 equiv) in MeCN (26 mL) was added HCl•MeOH (4 M, 52.7 mL, 15 equiv). The reaction was stirred at 25° C. for 2 hours. The mixture was concentrated. 15% NaOH aqueous solution (30 mL) was added dropwise under ice-water bath. The mixture was extracted with ethyl acetate (6×80 mL). The combined organic layers were dried over anhydrous sodium sulfate and concentrated to afford the title compound (4.3 g, 94% yield) as yellow oil; ¹H NMR (400 MHz, CHLOROFORM-d1) δ4.91 (s, 2H), 4.09-4.01 (m, 2H), 3.96 (s, 3H), 3.86 (s, 2H), 3.73-3.65 (m, 1H), 3.26 (br d, J=14.0 Hz, 1H), 3.18-3.12 (m, 1H), 3.12-3.03 (m, 2H), 2.76 (br d, J=15.6 Hz, 1H), 2.68-2.59 (m, 1H), 2.51 (t, J=5.6 Hz, 2H), 2.36 (br d, J=15.6 Hz, 1H), 2.19-2.09 (m, 1H), 1.96-1.83 (m, 2H), 1.79-1.75 (m, 2H).

Step E. (S)-7-(8-ethyl-7-fluoro-3-(methoxymethoxy)naphthalen-1-yl)-4-methoxy-2-((2-methylenetetrahydro-1H-pyrrolizin-7a(5H)-yl)methoxy)-5,6,7,8-tetrahydropyrido[3,4-d] pyrimidine: To a mixture of (S)-4-methoxy-2-((2-methylenetetrahydro-1H-pyrrolizin-7a(5H)-yl)methoxy)-5,6,7,8-tetrahydropyrido[3,4-d]pyrimidine (0.2 g, 1 equiv) and 8-ethyl-7-fluoro-3-(methoxymethoxy)naphthalen-1-yl trifluoromethanesulfonate (314 mg, 1.3 equiv) in dioxane (5 mL) were added 1,3-bis[2,6-bis(1-propylbutyl)phenyl]-4,5-dichloro-2H-imidazol-1-ium-2-ide;3-chloropyridine;dichloropalladium (61.5 mg, 0.1 equiv) and Cs₂CO₃ (515 mg, 2.5 equiv). The reaction was degassed and purged with nitrogen 3 times. The reaction was stirred at 90° C. for 12 hours. The mixture was filtered, concentrated and purified by reversed phase flash chromatography [C18, 0.1% formic acid condition] to afford the title compound (0.1 g, 27% yield) as yellow solid; ¹HNMR (400 MHz, CHLOROFORM-d1) δ 7.59-7.52 (m, 1H), 7.23-7.15 (m, 2H), 7.09-7.03 (m, 1H), 5.30-5.25 (m, 2H), 4.95-4.88 (m, 2H), 4.09-4.06 (m, 2H), 4.03 (s, 3H), 3.80-3.73 (m, 1H), 3.70 (br d, J=14.0 Hz, 1H), 3.55-3.52 (m, 3H), 3.52-3.46 (m, 1H), 3.38-3.29 (m, 2H), 3.29-3.18 (m, 2H), 3.18-3.11 (m, 1H), 2.95-2.83 (m, 1H), 2.82-2.74 (m, 1H), 2.73-2.60 (m, 2H), 2.41-2.33 (m, 1H), 2.22-2.11 (m, 1H), 1.96-1.84 (m, 2H), 1.80-1.69 (m, 1H), 1.67-1.61 (m, 1H), 1.06 (t, J=7.2 Hz, 3H); LCMS (ESI, M+1): m/z=549.3.

Step F. (S)-7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d]pyrimidin-4-ol: To a solution of (S)-7-(8-ethyl-7-fluoro-3-(methoxymethoxy)naphthalen-1-yl)-4-methoxy-2-((2-methylenetetrahydro-1H-pyrrolizin-7a(5H)-yl)methoxy)-5,6,7,8-tetrahydropyrido[3,4-d]pyrimidine (300 mg, 1 equiv) in DMF (3 mL) was added NaSEt (184 mg, 4 equiv). The reaction was stirred at 60° C. for 1 hour. The mixture was diluted with water (6 mL) and extracted with ethyl acetate (3 mL×5). The combined organic layers were dried over anhydrous sodium sulfate, concentrated, and purified by reversed phase flash chromatography [C18, 0.1% formic acid condition] to afford the title compound (225 mg, 75% yield) as yellow solid; LCMS (ESI, M+1): m/z=535.2.

Step G. (R)-7-(7-(8-ethyl-7-fluoro-3-(methoxymethoxy)naphthalen-1-yl)-2-(((S)-2-methylenetetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione: To a solution of (S)-7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d]pyrimidin-4-ol (235 mg, 1 equiv) in DMSO (2.35 mL) were added TEA (133 mg, 3 equiv) and PyBOP (343 mg, 1.5 equiv). The mixture was stirred at 25° C. for 20 minutes, and then (R)-1,3,7-triazaspiro[4.5]decane-2,4-dione (97 mg, 1.3 equiv) was added. The reaction was stirred at 25° C. for 2 hours. The mixture was filtered and purified with reversed phase flash chromatography [C18, 0.1% formic acid condition] to afford the title compound (290 mg, 86% yield) as yellow solid.

Step H. (R)-7-(7-(8-ethyl-7-fluoro-3-hydroxynaphthalen-1-yl)-2-(((S)-2-methylenetetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione: To a solution of (R)-7-(7-(8-ethyl-7-fluoro-3-(methoxymethoxy)naphthalen-1-yl)-2-(((S)-2-methylenetetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (250 mg, 1 equiv) in MeOH (0.9 mL) was added HCl-MeOH (4 M, 1.82 mL, 20 equiv) dropwise at 5° C. The reaction was stirred at 5° C. for 0.5 hour. The mixture was concentrated at room temperature. The residue was dissolved in MeOH (3 mL) under ice-water bath, neutralized with solid NaHCO₃, and filtered. The filtrate was purified by reversed phase flash chromatography [C18, 0.05% NH₃•H₂O condition] to afford 93 mg of the crude product. 30 mg of the crude product was purified by prep-HPLC [Waters Xbridge 150×25 mm×5 μm; A: water (NH₄HCO₃); B: ACN, B %:42%-72% over 9 min] to afford the title compound (7.51 mg, 9.6% yield) as yellow solid. ¹H NMR (400 MHz, METHANOL-d₄) δ 7.58-7.48 (m, 1H), 7.16 (t, J=9.6 Hz, 1H), 7.06-6.95 (m, 2H), 5.01-4.94 (m, 2H), 4.24-4.10 (m, 4H), 4.08-3.90 (m, 1H), 3.77-3.60 (m, 2H), 3.57-3.34 (m, 5H), 3.26-2.99 (m, 4H), 2.87-2.64 (m, 3H), 2.43 (br d, J=15.6 Hz, 1H), 2.20-2.07 (m, 2H), 2.06-1.76 (m, 6H), 1.17-1.08 (m, 3H); LCMS (ESI+1): m/z=642.2.

### Example 585B

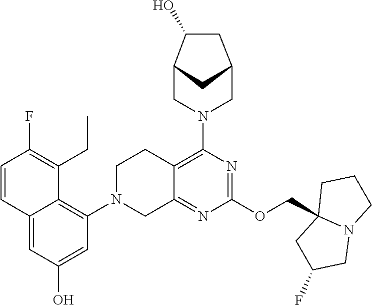 [SMILES: CCc1c(F)ccc2cc(O)cc(N3CCc4c(nc(OC[C@@]56CCCN5C[C@H](F)C6)nc4N4C[C@@H]5C[C@H](C4)[C@H](O)C5)C3)c12]

### (1R,5R,6R)-3-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-3-azabicyclo[3.2.1]octan-6-ol

Synthesized according to Example 248. The title compound was obtained as yellow solid (FA salt). ¹H NMR (400 MHz, METHANOL-d₄) δ=7.56-7.45 (m, 1H), 7.14 (t, J=9.2 Hz, 1H), 7.03-6.91 (m, 2H), 5.55-5.32 (m, 1H), 4.69-4.51 (m, 1H), 4.42-3.81 (m, 4H), 4.07 (br dd, J=3.6, 17.6 Hz, 1H), 3.69-3.52 (m, 4H), 3.51-3.33 (m, 4H), 3.29-3.10 (m, 3H), 3.10-2.70 (m, 2H), 2.57-2.36 (m, 2H), 2.32-2.11 (m, 6H), 2.08-1.98 (m, 1H), 1.82-1.68 (m, 2H), 1.62-1.22 (m, 1H), 1.11 (q, J=7.2 Hz, 3H); LCMS (ESI, M+1): m/z=606.3.

### Example 586

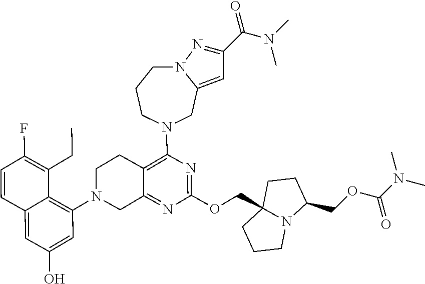 [SMILES: CCc1c(F)ccc2cc(O)cc(N3CCc4c(nc(OC[C@]56CCCN5[C@H](COC(=O)N(C)C)CC6)nc4N4CCCn5nc(C(=O)N(C)C)cc5C4)C3)c12]

### ((3S,7aR)-7a-(((4-(2-(dimethylcarbamoyl)-7,8-dihydro-4H-pyrazolo[1,5-a][1,4]diazepin-5(6H)-yl)-7-(8-ethyl-7-fluoro-3-hydroxynaphthalen-1-yl)-5,6,7,8-tetrahydropyrido[3,4-d]pyrimidin-2-yl)oxy)methyl)hexahydro-1H-pyrrolizin-3-yl)methyl dimethylcarbamate

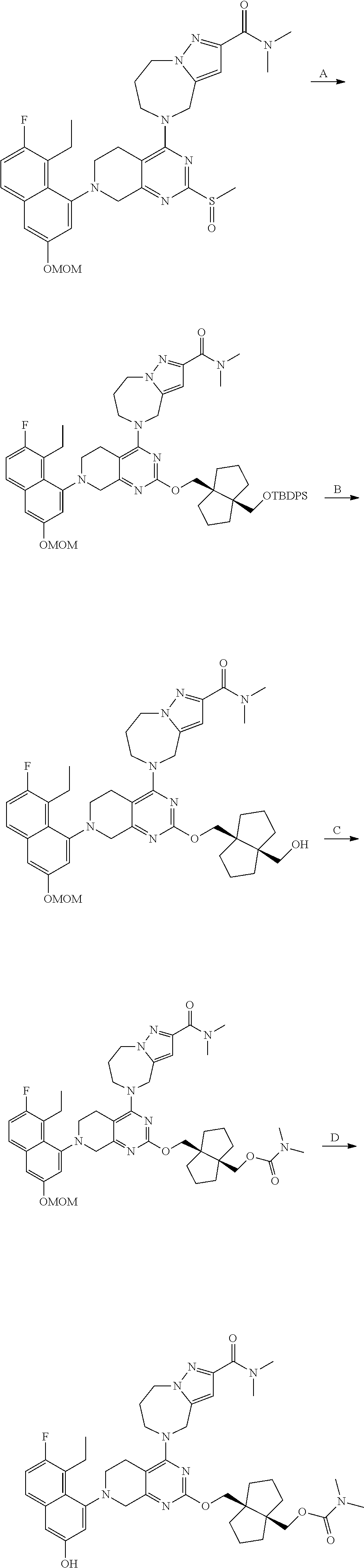

Step A. 5-(2-(((3S,7aR)-3-(((tert-butyldiphenylsilyl)oxy)methyl)hexahydro-1H-pyrrolizin-7a-yl)methoxy)-7-(8-ethyl-7-fluoro-3-(methoxymethoxy)naphthalen-1-yl)-⁵,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide: To a solution of 5-(7-(8-ethyl-7-fluoro-3-(methoxymethoxy)naphthalen-1-yl)-2-(methylsulfinyl)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide (800 mg, 1.0 equiv) and ((3S,7aR)-3-(((tert-butyldiphenylsilyl)oxy)methyl)hexahydro-1H-pyrrolizin-7a-yl)methanol (515 mg, 1.0 equiv) in THF (20 mL) was added t-BuONa (363 mg, 3.0 equiv) in portions at 0° C. The reaction was stirred at 25° C. for 1 hour. The mixture was quenched with ice-water (w/w=1/1, 10 mL) and extracted with ethyl acetate (3×15 mL). The combined organic layers were washed with brine (25 mL), dried with sodium sulfate, filtered, concentrated and purified by reversed-phase HPLC (0.1% FA condition) to afford the title compound (1.45 g, 36% yield) as yellow solid; LCMS (ESI, M+1): m/z=981.5.

Step B. 5-(7-(8-ethyl-7-fluoro-3-(methoxymethoxy)naphthalen-1-yl)-2-(((3S,7aR)-3-(hydroxymethyl)hexahydro-1H-pyrrolizin-7a-yl)methoxy)-5,6,7,8-tetrahydropyrido[3,4-d] pyrimidin-4-yl)-N,N-dimethyl-5,6,7,8-tetrahydro-4H-pyrazolo[1,5-a][1,4]diazepine-2-carboxamide: To a solution of 5-(2-(((3S,7aR)-3-(((tert-butyldiphenylsilyl)oxy)methyl)hexahydro-1H-pyrrolizin-7a-yl)methoxy)-7-(8-ethyl-7-fluoro-3-(methoxymethoxy)naphthalen-1-yl)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide (400 mg, 1.0 equiv) in DMF (5 mL) was added CsF (929 mg, 15.0 equiv). The reaction was stirred at 25° C. for 12 hours. The mixture was quenched with ethyl acetate (3×20 mL). The combined organic layers were washed with brine (25 mL), dried over sodium sulfate, filtered, concentrated and purified by prep-HPLC (column: Phenomenex luna C18 150*25 mm*10 μm; mobile phase: [water (FA)-ACN]; B %: 17%-47%, 10 min) to afford the title compound (100 mg, 33% yield) as yellow solid; LCMS (ESI, M+1): m/z=743.4.

Step C. ((3S,7aR)-7a-(((4-(2-(dimethylcarbamoyl)-7,8-dihydro-4H-pyrazolo[1,5-a][1,4]diazepin-5(6H)-yl)-7-(8-ethyl-7-fluoro-3-(methoxymethoxy)naphthalen-1-yl)-5,6,7,8-tetrahydropyrido[3,4-d] pyrimidin-2-yl)oxy)methyl)hexahydro-1H-pyrrolizin-3-yl)methyl dimethylcarbamate: To a solution of 5-(7-(8-ethyl-7-fluoro-3-(methoxymethoxy)naphthalen-1-yl)-2-(((3S,7aR)-3-(hydroxymethyl)hexahydro-1H-pyrrolizin-7a-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide (80.0 mg, 1.0 equiv) and N,N-dimethylcarbamoyl chloride (34.7 mg, 3.0 equiv) in THF (1 mL) was added NaH (21.5 mg, 60% purity, 5.0 equiv) in portions at 0° C. The reaction was stirred at 25° C. for 1 hour. The mixture was quenched with ice-water (w/w=1/1, 10 mL) and extracted with ethyl acetate (3×10 mL). The combined organic layers were washed with brine (20 mL), dried over sodium sulfate, filtered and concentrated to afford the title compound (80.0 mg, crude) as yellow oil; LCMS (ESI, M+1): m/z=814.4.

Step D. ((3S,7aR)-7a-(((4-(2-(dimethylcarbamoyl)-7,8-dihydro-4H-pyrazolo[1,5-a][1,4]diazepin-5(6H)-yl)-7-(8-ethyl-7-fluoro-3-hydroxynaphthalen-1-yl)-5,6,7,8-tetrahydropyrido[3,4-d]pyrimidin-2-yl)oxy)methyl)hexahydro-1H-pyrrolizin-3-yl)methyl dimethylcarbamate: A mixture of ((3S,7aR)-7a-(((4-(2-(dimethylcarbamoyl)-7,8-dihydro-4H-pyrazolo[1,5-a][1,4]diazepin-5(6H)-yl)-7-(8-ethyl-7-fluoro-3-(methoxymethoxy)naphthalen-1-yl)-5,6,7,8-tetrahydropyrido[3,4-d]pyrimidin-2-yl)oxy)methyl)hexahydro-1H-pyrrolizin-3-yl)methyl dimethylcarbamate (80.0 mg, 1.0 equiv) in HCl•MeOH (1 mL) was stirred at 25° C. for 1 hour. The mixture was concentrated and purified by prep-HPLC (column: Phenomenex Luna C18 150*25 mm*10 μm; mobile phase: [water (FA)-ACN]; B %: 15%-45%, 10 min) to afford the title compound (45.5 mg, FA salt) as yellow solid. ¹H NMR (400 MHz, METHANOL-d₄) δ=8.55 (br s, 1H), 7.53 (dd, J=6.0, 9.2 Hz, 1H), 7.16 (t, J=9.2 Hz, 1H), 6.99 (s, 2H), 6.62 (s, 1H), 5.06-4.95 (m, 1H), 4.87-4.82 (m, 1H), 4.70-4.49 (m, 3H), 4.26-4.15 (m, 4H), 4.15-3.99 (m, 3H), 3.69 (br d, J=17.6 Hz, 1H), 3.55 (br d, J=10.4 Hz, 1H), 3.40 (br d, J=7.2 Hz, 2H), 3.31-3.14 (m, 4H), 3.09 (s, 4H), 2.96-2.84 (m, 6H), 2.74 (br d, J=14.0 Hz, 1H), 2.38-2.20 (m, 2H), 2.16-1.74 (m, 9H), 1.13 (t, J=7.2 Hz, 3H); LCMS (ESI, M+1): m/z=770.3.

### Example 587

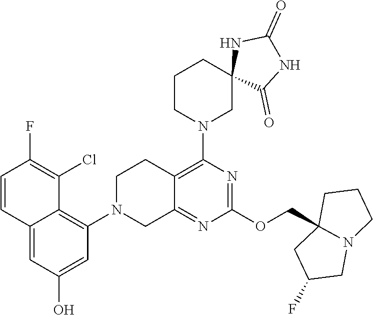 [SMILES: O=C1NC(=O)[C@]2(CCCN(c3nc(OC[C@@]45CCCN4C[C@H](F)C5)nc4c3CCN(c3cc(O)cc5ccc(F)c(Cl)c35)C4)C2)N1]

### (R)-7-(7-(8-chloro-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione

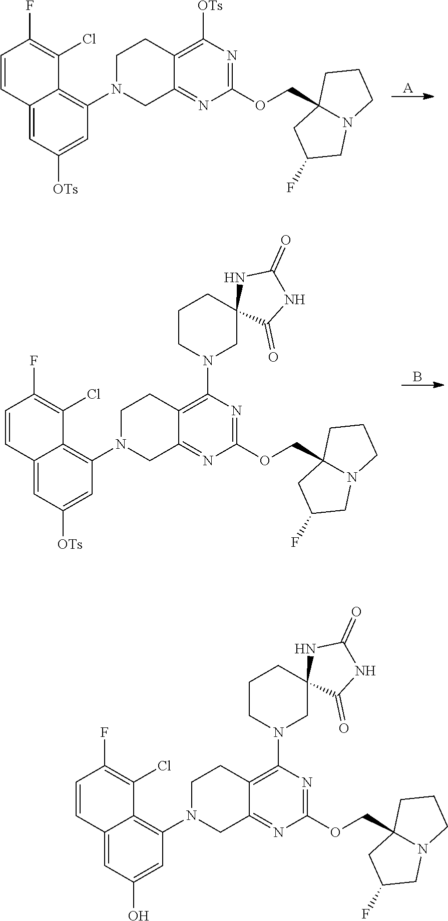

Step A. 5-chloro-4-(4-((R)-2,4-dioxo-1,3,7-triazaspiro[4.5]decan-7-yl)-2-(((2R,7aS)-2-fluorohexahydro-1H-pyrrolizin-7a-yl)methoxy)-5,6-dihydropyrido[3,4-d]pyrimidin-7(8H)-yl)-6-fluoronaphthalen-2-yl 4-methylbenzenesulfonate: To a solution of 5-chloro-6-fluoro-4-(2-(((2R,7aS)-2-fluorohexahydro-1H-pyrrolizin-7a-yl)methoxy)-4-(tosyloxy)-5,6-dihydropyrido[3,4-d]pyrimidin-7(8H)-yl)naphthalen-2-yl 4-methylbenzenesulfonate (40.0 mg, 1.0 equiv) and (5R)-1,3,9-triazaspiro[4.5]decane-2,4-dione (25.0 mg, 3.0 equiv) in DMF (1.0 mL) was added DIEA (31.9 mg, 5.0 equiv). The mixture was stirred at 60° C. for 2 hour. The mixture was filtered and purified with prep-TLC (SiO₂, Petroleum ether/Ethyl acetate=1/1) to afford the title compound as white solid; LCMS (ESI, M+1): m/z=808.4.

Step B. (R)-7-(7-(8-chloro-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione: To a solution of 5-chloro-4-(4-((R)-2,4-dioxo-1,3,7-triazaspiro[4.5]decan-7-yl)-2-(((2R,7aS)-2-fluorohexahydro-1H-pyrrolizin-7a-yl)methoxy)-5,6-dihydropyrido[3,4-d]pyrimidin-7(8H)-yl)-6-fluoronaphthalen-2-yl 4-methylbenzenesulfonate (10.0 mg, 1.0 equiv) in MeOH (1.0 mL) was added NaOH (9.90 mg, 20 equiv). The mixture was stirred at 20° C. for 0.5 hour. The mixture was diluted with ethyl acetate (10 mL) and washed with brine (5 mL×2). The organic layer were dried over anhydrous sodium sulfate, filtered, concentrated and purified with prep-HPLC [column: 3_Phenomenex Luna C18 75×30 mm×3 μm;mobile phase: [water(FA)-ACN];B %: 18%-38%,8 min] to afford the title compound as white solid (FA salt). 1H NMR (400 MHz, METHANOL-d₄) δ=8.49 (br s, 1H), 7.63 (dd, J=5.6, 9.2 Hz, 1H), 7.32-7.23 (m, 1H), 7.04-6.86 (m, 2H), 5.52-5.33 (m, 1H), 4.39-4.23 (m, 3H), 4.20-3.93 (m, 2H), 3.82-3.48 (m, 5H), 3.40 (br dd, J=5.4, 13.2 Hz, 2H), 3.24-2.98 (m, 3H), 2.81-2.59 (m, 1H), 2.37-1.74 (m, 8H); LCMS (ESI, M+1): m/z=654.4.

### Example 588

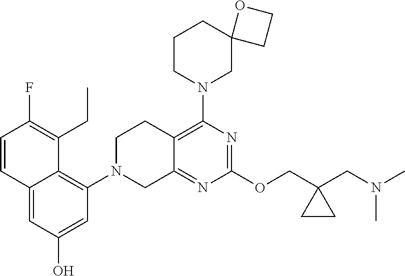 [SMILES: CCc1c(F)ccc2cc(O)cc(N3CCc4c(nc(OCC5(CN(C)C)CC5)nc4N4CCCC5(CCO5)C4)C3)c12]

### 4-(2-((1-((dimethylamino)methyl)cyclopropyl)methoxy)-4-(1-oxa-6-azaspiro[3.5]nonan-6-yl)-5,8-dihydropyrido[3,4-d]pyrimidin-7(6H)-yl)-5-ethyl-6-fluoronaphthalen-2-ol

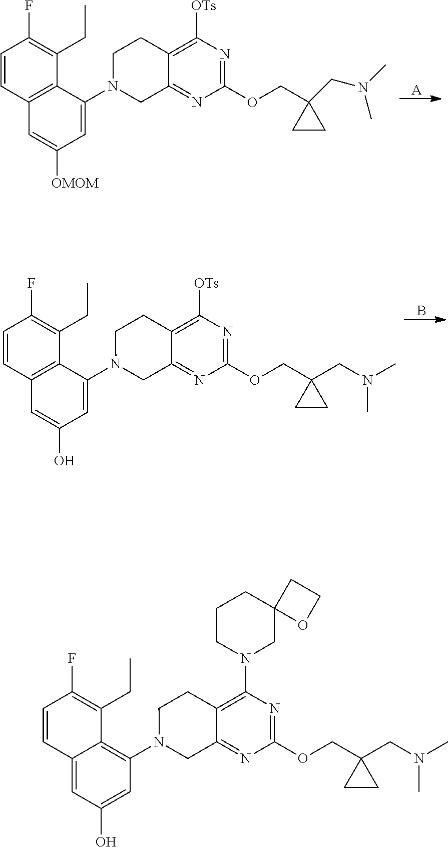

Step A. 2-((1-((dimethylamino)methyl)cyclopropyl)methoxy)-7-(8-ethyl-7-fluoro-3-hydroxynaphthalen-1-yl)-⁵,6,7,8-tetrahydropyrido[3,4-d]pyrimidin-4-yl4-methylbenzenesulfonate: To a solution of 2-((1-((dimethylamino)methyl)cyclopropyl)methoxy)-7-(8-ethyl-7-fluoro-3-(methoxymethoxy)naphthalen-1-yl)-5,6,7,8-tetrahydropyrido[3,4-d]pyrimidin-4-yl 4-methylbenzenesulfonate (50.0 mg, 1.0 equiv) in DCM (0.5 mL) was added TFA (129 mg, 15 equiv). The reaction was stirred at 0° C. for 2 hours. The mixture was quenched with NH₃•H₂O (1 mL), H₂O (5 mL) and extracted with DCM (3 mL). The organic layer was dried over sodium sulfate, filtered, concentrated under reduced pressure and purified by prep-HPLC (column: Phenomenex luna C18 150*25 mm*10 μm; mobile phase: [water (FA)-ACN]; B %: 25%-55%, 10 min) to afford the title compound (12.0 mg, 26% yield) as white solid. ¹H NMR (400 MHz, METHANOL-d₄) δ=8.69-8.38 (m, 1H), 7.53 (dd, J=5.6, 8.8 Hz, 1H), 7.17 (t, J=9.2 Hz, 1H), 7.03-6.95 (m, 2H), 4.72-4.51 (m, 4H), 4.31-4.21 (m, 2H), 4.21-3.93 (m, 2H), 3.84-3.61 (m, 2H), 3.60-3.43 (m, 3H), 3.28-2.98 (m, 3H), 2.87-2.65 (m, 3H), 2.54 (br d, J=9.6 Hz, 6H), 2.49-2,13 (m, 3H), 2.04-1.95 (m, 1H), 1.89-1.57 (m, 2H), 1.23-1.05 (m, 3H), 0.77 (br d, J=2.8 Hz, 2H), 0.66-0.53 (m, 2H); ¹⁹F NMR (376 MHz, METHANOL-d₄) δ=−123.06 (br d, J=14.3 Hz, 1F); LCMS (ESI, M+1): m/z=621.4.

Step B. 4-(2-((1-((dimethylamino)methyl)cyclopropyl)methoxy)-4-(1-oxa-6-azaspiro[3.5]nonan-6-yl)-5,6-dihydropyrido[3,4-d]pyrimidin-7(8H)-yl)-5-ethyl-6-fluoronaphthalen-2-ol: To a mixture of 2-((1-((dimethylamino)methyl)cyclopropyl)methoxy)-7-(8-ethyl-7-fluoro-3-hydroxynaphthalen-1-yl)-5,6,7,8-tetrahydropyrido[3,4-d]pyrimidin-4-yl 4-methylbenzenesulfonate (10.0 mg, 1.0 equiv) and 1-oxa-6-azaspiro[3.5]nonane (8.32 mg, 3.0 equiv, 0.5 oxalic salt) in DMF (0.5 mL) were added DIEA (6.25 mg, 3.0 equiv) and 4 Å molecular sieve (10 mg). The reaction was stirred at 40° C. for 12 hours. The mixture was filtered and purified by prep-HPLC (column: Phenomenex luna C18 150*25 mm*10 μm; mobile phase: [water (FA)-ACN]; B %: 16%-46%, 10 min) to afford the title compound (6.00 mg, FA salt) as a white solid. ¹H NMR (400 MHz, METHANOL-d₄) δ=8.69-8.38 (m, 1H), 7.53 (dd, J=5.6, 8.8 Hz, 1H), 7.17 (t, J=9.2 Hz, 1H), 7.03-6.95 (m, 2H), 4.72-4.51 (m, 4H), 4.31-4.21 (m, 2H), 4.21-3.93 (m, 2H), 3.84-3.61 (m, 2H), 3.60-3.43 (m, 3H), 3.28-2.98 (m, 3H), 2.87-2.65 (m, 3H), 2.54 (br d, J=9.6 Hz, 6H), 2.49-2.13 (m, 3H), 2.04-1.95 (m, 1H), 1.89-1.57 (m, 2H), 1.23-1.05 (m, 3H), 0.77 (br d, J=2.8 Hz, 2H), 0.66-0.53 (m, 2H); ¹⁹F NMR (376 MHz, METHANOL-d₄) δ=−123.06 (br d, J=14.3 Hz, 1F); LCMS (ESI, M+1): m/z=576.4.

### Example 589

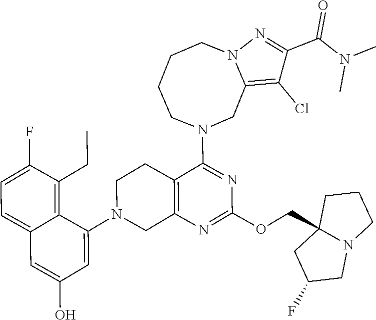 [SMILES: CCc1c(F)ccc2cc(O)cc(N3CCc4c(nc(OC[C@@]56CCCN5C[C@H](F)C6)nc4N4CCCCn5nc(C(=O)N(C)C)c(Cl)c5C4)C3)c12]

### 3-chloro-5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-N,N-dimethyl-4,5,6,7,8,9-hexahydropyrazolo[1,5-a][1,4] diazocine-2-carboxamide

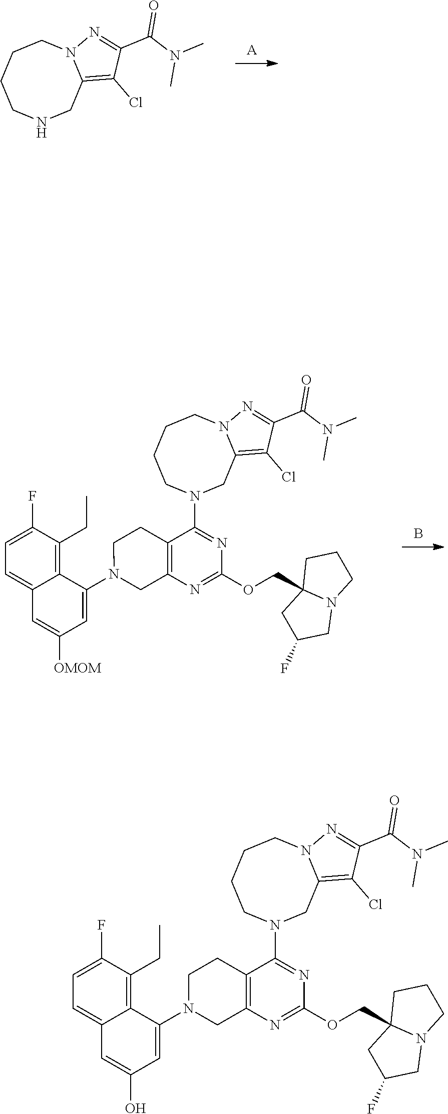

Step A. 3-chloro-5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-N,N-dimethyl-4,5,6,7,8,9-hexahydropyrazolo[1,5-a][1,4] diazocine-2-carboxamide: To a solution of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (150 mg, 1.0 equiv) and 3-chloro-N,N-dimethyl-4,5,6,7,8,9-hexahydropyrazolo[1,5-a][1,4] diazocine-2-carboxamide (160 mg, 2.0 equiv, TFA salt) in DMF (1.5 mL) was added DIEA (140 mg, 5.0 equiv) at 25° C. The reaction was stirred at 80° C. for 12 hours. The mixture was concentrated and purified by reversed-phase column chromatography (column: Phenomenex luna C18 150*25 mm*10 μm;mobile phase: [water(FA)-ACN];B %: 22%-55%,10 min) to afford the title compound (110 mg, 65% yield) as light yellow solid.

Step B. 3-chloro-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-N,N-dimethyl-4,5,6,7,8,9-hexahydropyrazolo[1,5-a][1,4] diazocine-2-carboxamide: To a solution of 3-chloro-5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-N,N-dimethyl-4,5,6,7,8,9-hexahydropyrazolo[1,5-a][1,4] diazocine-2-carboxamide (60.0 mg, 1.0 equiv) in MeOH (0.5 mL) was added HCl•MeOH (4 M, 1 mL, 52.0 equiv). The reaction was stirred at 0° C. for 2 hours. The mixture was concentrated and purified by reversed-phase column chromatography (column: Waters Xbridge 150*25 mm*Sum;mobile phase: [water(NH₄HCO₃)-ACN]; B %: 46%-76%, 8 min) to afford the title compound (30.0 mg, 53% yield) as light yellow solid. ¹H NMR (400 MHz, METHANOL-d₄) δ=1.07-1.16 (m, 3H) 1.82 (br dd, J=12.4, 11.2 Hz, 2H) 1.87-2.05 (m, 6H) 2.06-2.28 (m, 2H) 2.68 (br d, J=14.6 Hz, 1H) 2.91-3.03 (m, 1H) 3.08-3.14 (m, 7H) 3.16-3.22 (m, 3H) 3.23-3.28 (m, 1H) 3.35-3.54 (m, 4H) 3.60-3.77 (m, 2H) 3.84-4.14 (m, 4H) 4.34 (br d, J=3.6 Hz, 2H) 4.92 (br s, 1H) 5.16-5.35 (m, 1H) 6.91-7.02 (m, 2H) 7.10 (s, 1H) 7.51 (dd, J=9.0, 6.0 Hz, 1H); LCMS (ESI, M+1): m/z=735.3.

### Example 590

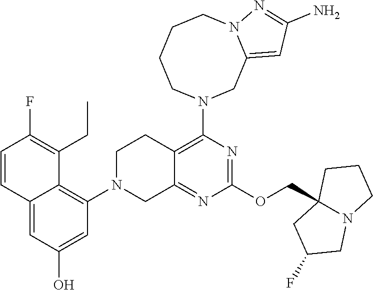 [SMILES: CCc1c(F)ccc2cc(O)cc(N3CCc4c(nc(OC[C@@]56CCCN5C[C@H](F)C6)nc4N4CCCCn5nc(N)cc5C4)C3)c12]

### 4-(4-(2-amino-6,7,8,9-tetrahydropyrazolo[1,5-a][1,4] diazocin-5(4H)-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-ol

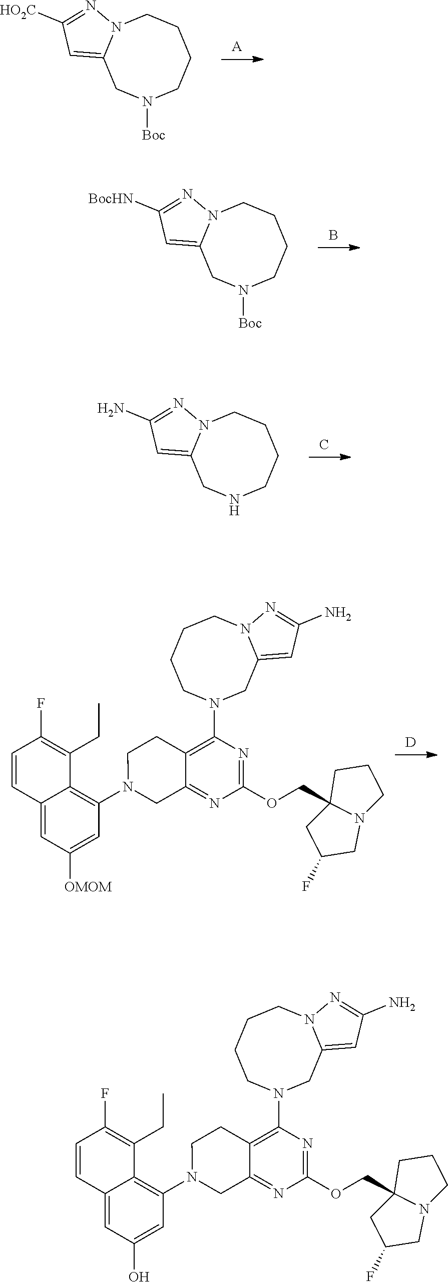

Step A. tert-butyl 2-((tert-butoxycarbonyl)amino)-6,7,8,9-tetrahydropyrazolo[1,5-a][1,4]diazocine-5(4H)-carboxylate: To a mixture of 5-(tert-butoxycarbonyl)-4,5,6,7,8,9-hexahydropyrazolo[1,5-a][1,4] diazocine-2-carboxylic acid (500 mg, 1.0 equiv), 4 Å molecular sieve (300 mg) and TEA (514 mg, 3.0 equiv) in toluene (5.0 mL) were added DPPA (699 mg, 1.5 equiv) and t-BuOH (3.76 g, 30 equiv). The reaction was stirred at 110° C. for 5 hours under N₂ atmosphere. The mixture was diluted with water (15 mL) and extracted with ethyl acetate (3×10 mL). The combined organic layers were washed with brine (2×10 mL), dried over Na₂SO₄, concentrated and purified by column chromatography (SiO₂, petroleum ether/Ethyl acetate=10/1 to 2/1) to afford the title compound (460 mg, 73% yield) as an off-white solid. ¹H NMR (400 MHz, chloroform-d) δ=6.37 (s, 1H), 4.53 (br d, J=16.8 Hz, 2H), 4.22-4.05 (m, 2H), 3.43-3.15 (m, 2H), 1.89-1.75 (m, 2H), 1.66-1.59 (m, 2H), 1.57-1.42 (m, 18H); LCMS (ESI, M+1): m/z=367.1.

Step B. 4,5,6,7,8,9-hexahydropyrazolo[1,5-a][1,4]diazocin-2-amine: A solution of tert-butyl 2-((tert-butoxycarbonyl)amino)-6,7,8,9-tetrahydropyrazolo[1,5-a][1,4] diazocine-5(4H)-carboxylate (460 mg, 1.0 equiv) in HCl-MeOH (4.6 mL) was stirred at 20° C. for 2 hours. The mixture was concentrated to afford the title compound (317 mg, crude, HCl salt) as an off-white solid. ¹H NMR (400 MHz, DMSO-d₆) δ=9.59 (br s, 2H), 6.33 (s, 1H), 4.40-4.33 (m, 4H), 2.88 (br s, 2H), 1.95-1.43 (m, 4H); LCMS (ESI, M+1): m/z=167.1.

Step C. 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-4,5,6,7,8,9-hexahydropyrazolo[1,5-a][1,4] diazocin-2-amine: To a solution of 7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (200 mg, 1.0 equiv) and DIEA (186 mg, 5.0 equiv) in DMF (2 mL) were added 4,5,6,7,8,9-hexahydropyrazolo[1,5-a][1,4] diazocin-2-amine (117 mg, 2.0 equiv, HCl salt) and 4 Å molecular sieve (30 mg). The reaction was stirred at 40° C. for 20 hours. The mixture was filtered, diluted with water (10 mL) and extracted with ethyl acetate (3×5 mL). The combined organic layers were washed with brine (2×5 mL), dried over Na₂SO₄, concentrated and purified by reversed-phase flash (0.1% FA condition) to afford the title compound (90 mg, 38% yield) as a light yellow oil; LCMS (ESI, M+1): m/z=689.4.

Step D. 4-(4-(2-amino-6,7,8,9-tetrahydropyrazolo[1,5-a][1,4] diazocin-5(4H)-yl)-2-(((2R,7aS)-2-fluorotetrahydro-11H-pyrrolizin-7a(5H)-yl)methoxy)-5,8-dihydropyrido[3,4-d]pyrimidin-7(6H)-yl)-5-ethyl-6-fluoronaphthalen-2-ol: To a solution of 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-4,5,6,7,8,9-hexahydropyrazolo[1,5-a][1,4] diazocin-2-amine (90.0 mg, 1.0 equiv) in MeOH (0.3 mL) was added HCl•MeOH (4 M, 0.6 mL, 18 equiv) at 0° C. The reaction was stirred at 0° C. for 1 hour. The mixture was quenched with ice cooled saturated NaHCO₃ solution (5.00 mL) dropwise at 0° C. and extracted with ethyl acetate (3×3 mL). The combined organic layers were dried over Na₂SO₄, concentrated and purified by reversed-phase HPLC (column: Welch Ultimate C18 150×25 mm x Sum; mobile phase: [water (FA)-ACN]; B %: 15%-45%, 10 min) to afford the title compound (37.2 mg, 40.1% yield, HCOOH salt) as a yellow solid. ¹H NMR (400 MHz, METHANOL-d₄) δ=7.51 (dd, J=6.0, 9.2 Hz, 1H), 7.15 (t, J=9.6 Hz, 1H), 6.97 (s, 2H), 5.52 (s, 1H), 5.49-5.28 (m, 1H), 4.82-4.76 (m, 2H), 4.32-4.04 (m, 5H), 3.91-3.80 (m, 1H), 3.76-3.69 (m, 1H), 3.68-3.60 (m, 1H), 3.59-3.44 (m, 4H), 3.43-3.36 (m, 2H), 3.25-3.09 (m, 3H), 2.77-2.63 (m, 1H), 2.50-2.29 (m, 2H), 2.25-2.07 (m, 3H), 2.02-1.81 (m, 3H), 1.78-1.61 (m, 2H), 1.11 (t, J=6.8 Hz, 3H); LCMS (ESI, M+1): m/z=645.3.

### Example 591

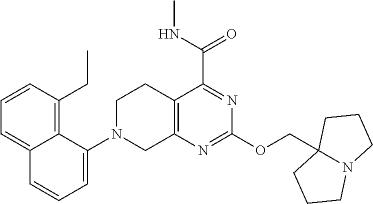 [SMILES: CCc1cccc2cccc(N3CCc4c(nc(OCC56CCCN5CCC6)nc4C(=O)NC)C3)c12]

### 7-(8-ethylnaphthalen-1-yl)-N-methyl-2-((tetrahydro-1H-pyrrolizin-7a(5H)-yl)methoxy)-5,6,7,8-tetrahydropyrido[3,4-d]pyrimidine-4-carboxamide

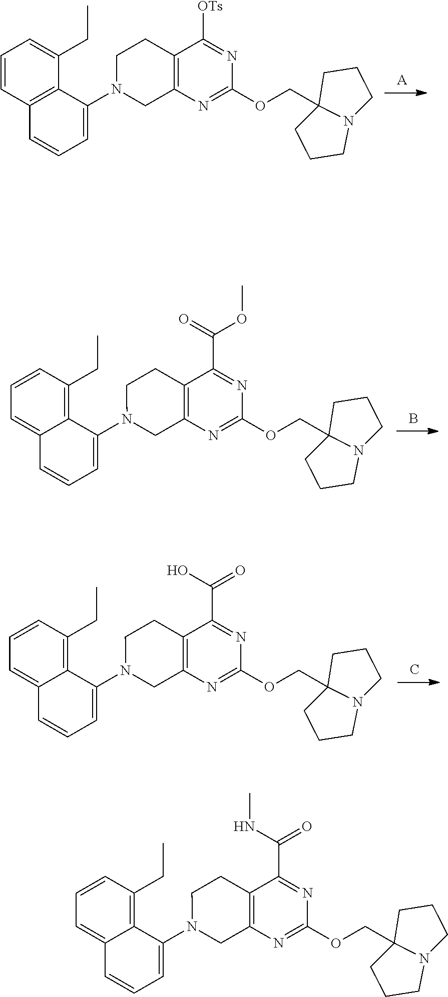

Step A. methyl 7-(8-ethylnaphthalen-1-yl)-2-((hexahydro-1H-pyrrolizin-7a-yl)methoxy)-5,6,7,8-tetrahydropyrido[3,4-d]pyrimidine-4-carboxylate: To a mixture of 7-(8-ethylnaphthalen-1-yl)-2-((hexahydro-1H-pyrrolizin-7a-yl)methoxy)-5,6,7,8-tetrahydropyrido[3,4-d]pyrimidin-4-yl 4-methylbenzenesulfonate (300 mg, 1.0 equiv), 1,3-Bis(diphenylphosphino)propane (41.3 mg, 0.2 equiv) and TEA (152 mg, 3.0 equiv) in MeOH (3 mL) and DMF (3 mL) was added Pd(OAc)₂ (11.2 mg, 0.1 equiv). The reaction was stirred at 80° C. for 24 hours under CO (50 Psi). The mixture was concentrated and diluted with water (30 mL). Then it was extracted with EtOAc (3×10 mL). The combined organic layers were dried over anhydrous sodium sulfate, filtered, and concentrated to afford the title compound (240 mg, crude) as a yellow solid.

Step B. 7-(8-ethylnaphthalen-1-yl)-2-((hexahydro-1H-pyrrolizin-7a-yl)methoxy)-5,6,7,8-tetrahydropyrido[3,4-d]pyrimidine-4-carboxylic acid: To a mixture of methyl 7-(8-ethylnaphthalen-1-yl)-2-((hexahydro-1H-pyrrolizin-7a-yl)methoxy)-5,6,7,8-tetrahydropyrido[3,4-d]pyrimidine-4-carboxylate (200 mg, 1.0 equiv) in THF (1 mL) and H₂O (0.2 mL) was added LiOH•H₂O (17.2 mg, 1.0 equiv). The reaction was stirred at 20° C. for 1 hour. The mixture was diluted with water (1 mL) and washed with EtOAc (1 mL×2). The aqueous layer was concentrated and purified by prep-HPLC [column: Phenomenex luna C18 150 * 25 mm*10 μm; mobile phase: [water (0.1% FA)-ACN]; B %: 26%-56%, 10 min] to afford the title compound (40.0 mg, 16% yield) as a yellow solid.

Step C. 7-(8-ethylnaphthalen-1-yl)-2-((hexahydro-1H-pyrrolizin-7a-yl)methoxy)-N-methyl-5,6,7,8-tetrahydropyrido[3,4-d] pyrimidine-4-carboxamide: To a mixture of 7-(8-ethylnaphthalen-1-yl)-2-((hexahydro-1H-pyrrolizin-7a-yl)methoxy)-5,6,7,8-tetrahydropyrido[3,4-d]pyrimidine-4-carboxylic acid (150 mg, 1.0 equiv), 4 Å molecular sieve (200 mg) and DIEA (205 mg, 5.0 equiv) in DMAc (1 mL) were added methanamine (42.9 mg, 2.0 equiv, HCl salt) and HATU (241 mg, 2.0 equiv). The reaction was stirred at 25° C. for 3 hours. The mixture was filtered and purified by prep-HPLC (column: YMC Triart C18 150*25 mm*5 μm; mobile phase: [water (HCl)-ACN];B %: 35%-65%,10 min]; B %: 28%-48%, 8 min) to afford the title compound (19.3 mg) as yellow solid. ¹HNMR (400 MHz, METHANOL-d₄) δ=7.71 (ddd, J=1.2, 8.0, 11.2 Hz, 2H), 7.48-7.41 (m, 1H), 7.41-7.32 (m, 2H), 7.31-7.25 (m, 1H), 4.69-4.56 (m, 3H), 4.19 (dd, J=1.2, 17.8 Hz, 1H), 3.92 (d, J=17.8 Hz, 1H), 3.72-3.63 (m, 2H), 3.62-3.55 (m, 1H), 3.51-3,36 (m, 3H), 3.30-3.25 (m, 2H), 3.05 (dd, J=7.2, 13.2 Hz, 1H), 2.97 (s, 3H), 2.39-2.28 (m, 2H), 2.28-2.14 (m, 4H), 2.14-2.06 (m, 2H), 1.08 (t, J=7.2 Hz, 3H); LCMS (ESI, M+1): m/z=486.3.

### Example 592

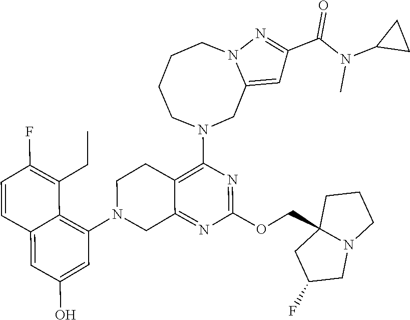 [SMILES: CCc1c(F)ccc2cc(O)cc(N3CCc4c(nc(OC[C@@]56CCCN5C[C@H](F)C6)nc4N4CCCCn5nc(C(=O)N(C)C6CC6)cc5C4)C3)c12]

### N-cyclopropyl-5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-N-methyl-4,5,6,7,8,9-hexahydropyrazolo[1,5-a][1,4] diazocine-2-carboxamide

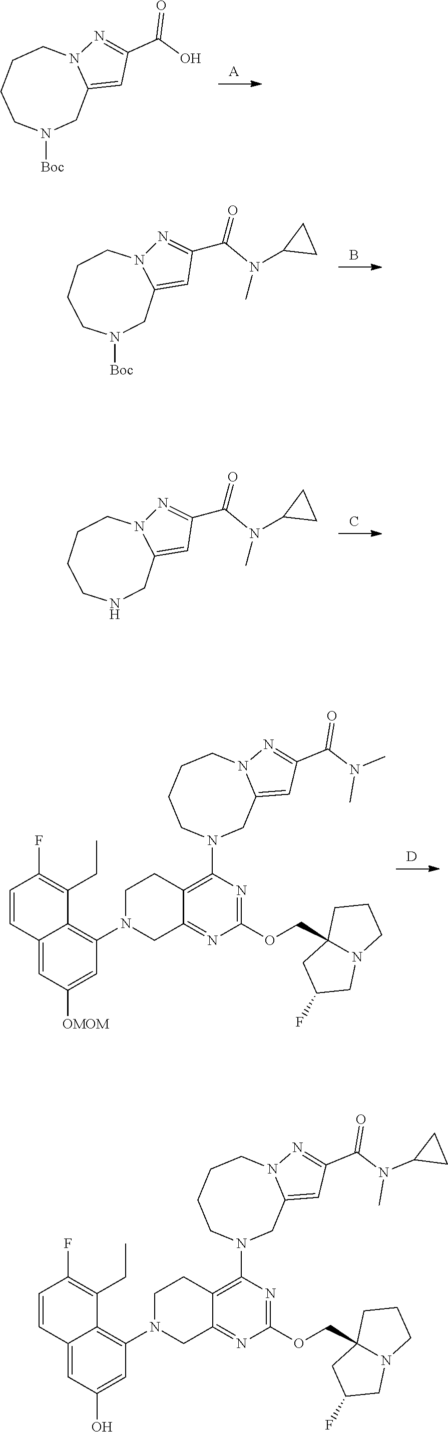

Step A. tert-butyl 2-(cyclopropyl(methyl)carbamoyl)-6,7,8,9-tetrahydropyrazolo[1,5-a][1,4] diazocine-5(4H)-carboxylate: To a mixture of 5-(tert-butoxycarbonyl)-4,5,6,7,8,9-hexahydropyrazolo[1,5-a][1,4] diazocine-2-carboxylic acid (400 mg, 1.0 equiv) and N-methylcyclopropanamine (218 mg, 1.5 equiv) in DCM (4 mL) were added HATU (772 mg, 1.5 eq) and TEA (548 mg, 4.0 equiv). The reaction was stirred at 20° C. for 2 hours under N₂. The mixture was diluted with water (100 mL) and extracted with ethyl acetate (3×100 mL). The combined organic layers were washed with brine (30 mL), dried over Na₂SO₄, filtered, concentrated under reduced pressure and purified by reversed phase flash [column: Phenomenex luna C18 150×25 mm×10 μm;mobile phase: water (0.1% formic acid)/acetonitrile)] to afford the title compound (470 mg, 99% yield) as white solid. LCMS (ESI, M+1): m/z=349.5.

Step B. N-cyclopropyl-N-methyl-4,5,6,7,8,9-hexahydropyrazolo[1,5-a][1,4]diazocine-2-carboxamide: A mixture of tert-butyl 2-(cyclopropyl(methyl)carbamoyl)-6,7,8,9-tetrahydropyrazolo[1,5-a][1,4] diazocine-5(4H)-carboxylate (470 mg, 1.0 equiv) and HCl-MeOH (4M, 5 mL) was stirred at 20° C. for 2 hours. The mixture was concentrated to afford the title compound (300 mg, 89% yield) as white solid.

Step C. N-cyclopropyl-5-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d] pyrimidin-4-yl)-N-methyl-4,5,6,7,8,9-hexahydropyrazolo[1,5-a][1,4]diazocine-2-carboxamide: To a mixture of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (100 mg, 1.0 equiv) and N-cyclopropyl-N-methyl-4,5,6,7,8,9-hexahydropyrazolo[1,5-a][1,4] diazocine-2-carboxamide (53.6 mg, 1.5 equiv) in THF (1 mL) were added DIEA (93.0 mg, 5 equiv) and 4 Å molecular sieve (50 mg). The reaction was stirred at 20° C. for 6 hours. The mixture is filtered and purified by prep-HPLC (column: Phenomenex luna C18 150×25 mm×10 μm; mobile phase: [water (FA)-ACN]; B %: 31%-61%,58 min) to afford the title compound (100 mg, 87% yield) as white solid. LCMS (ESI, M+1): m/z=771.5.

Step D. N-cyclopropyl-5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-N-methyl-4,5,6,7,8,9-hexahydropyrazolo[1,5-a][1,4]diazocine-2-carboxamide: A mixture of N-cyclopropyl-5-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-N-methyl-4,5,6,7,8,9-hexahydropyrazolo[1,5-a][1,4] diazocine-2-carboxamide (100 mg, 1.0 equiv) and HCl-MeOH (4M,1 mL) was stirred at 20° C. for 1 hour. The mixture was concentrated under reduced pressure and purified by prep-HPLC (column: Phenomenex luna C18 150×25 mm×10 μm; mobile phase: [water (FA)-ACN]; B %: 27%-57%, 58 min) to afford the title compound (4.4 mg) as an off-white solid. ¹H NMR (400 MHz, METHANOL-d₄) δ=7.52 (dd, J=5.6, 8.8 Hz, 1H), 7.15 (t, J=9.2 Hz, 1H), 7.01-6.93 (m, 2H), 6.54 (s, 1H), 5.41-5.19 (m, 1H), 5.07-4.93 (m, 2H), 4.54-4.45 (m, 2H), 4.24-3.96 (m, 4H), 3.91-3.82 (m, 1H), 3.79-3.68 (m, 2H), 3.54-3.46 (m, 1H), 3.45-3.35 (m, 3H), 3.19 (br d, J=8.4 Hz, 3H), 3.14-3.00 (m, 5H), 2.70 (br d, J=12.4 Hz, 1H), 2.38-2.24 (m, 1H), 2.23-2.17 (m, 1H), 2.12-2.06 (m, 1H), 2.04-1.94 (m, 4H), 1.89-1.79 (m, 2H), 1.77-1.64 (m, 1H), 1.16-1.09 (m, 3H), 0.73-0.61 (m, 2H), 0.60-0.38 (m, 2H); LCMS (ESI, M+1): m/z=727.4.

### Example 593

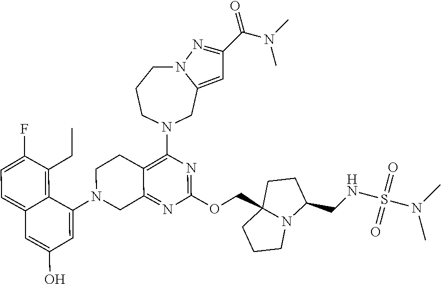 [SMILES: CCc1c(F)ccc2cc(O)cc(N3CCc4c(nc(OC[C@]56CCCN5[C@H](CNS(=O)(=O)N(C)C)CC6)nc4N4CCCn5nc(C(=O)N(C)C)cc5C4)C3)c12]

5-(2-(((3S,7aR)-3-(((N,N-dimethylsulfamoyl)amino)methyl)tetrahydro-1H-pyrrolizin-7a(5H)-yl)methoxy)-7-(8-ethyl-7-fluoro-3-hydroxynaphthalen-1-yl)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide

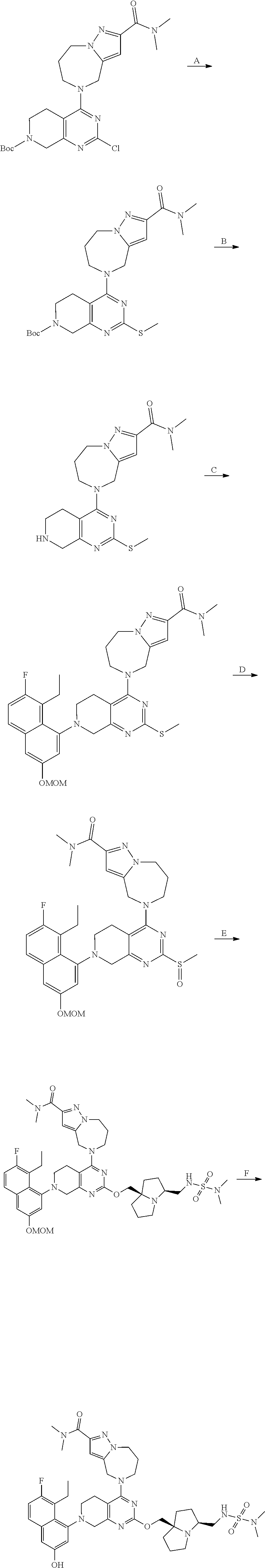

Step A. tert-butyl 4-(2-(dimethylcarbamoyl)-7,8-dihydro-4H-pyrazolo[1,5-a][1,4]diazepin-5(6H)-yl)-2-(methylthio)-5,6-dihydropyrido[3,4-d]pyrimidine-7(8H)-carboxylate: To a solution of tert-butyl 2-chloro-4-(2-(dimethylcarbamoyl)-7,8-dihydro-4H-pyrazolo[1,5-a][1,4]diazepin-5(6H)-yl)-5,6-dihydropyrido[3,4-d]pyrimidine-7(8H)-carboxylate (1.70 g, 1.0 equiv) in DMF (15 mL) was added NaSMe (751 mg, 3.0 equiv). The reaction was stirred at 25° C. for 1 hour. The mixture was poured into ice-water (w/w=1/1, 30 mL) and extracted with ethyl acetate (3×20 mL). The combined organic layers were washed with brine (50 mL), dried over sodium sulfate, filtered and concentrated to afford the title compound (1.70 g, 98% yield) as white solid. ¹HNMR (400 MHz, DMSO-d₆) δ=6.52 (s, 1H), 5.75 (s, 1H), 4.94-4.65 (m, 2H), 4.50-4.37 (m, 2H), 4.29 (br s, 2H), 3.89 (br s, 2H), 3.46 (br s, 2H), 3.24 (s, 3H), 2.93 (s, 3H), 2.68 (br t, J=4.3 Hz, 2H), 2.42-2.39 (m, 3H), 2.06 (br s, 2H), 1.44 (s, 9H); LCMS (ESI, M+1): m/z=488.3.

Step B. N,N-dimethyl-5-(2-(methylthio)-5,6,7,8-tetrahydropyrido[3,4-d] pyrimidin-4-yl)-5,6,7,8-tetrahydro-4H-pyrazolo[1,5-a][1,4]diazepine-2-carboxamide: To a solution of tert-butyl 4-(2-(dimethylcarbamoyl)-7,8-dihydro-4H-pyrazolo[1,5-a][1,4]diazepin-5(6H)-yl)-2-(methylthio)-5,6-dihydropyrido[3,4-d]pyrimidine-7(8H)-carboxylate (1.70 g, 1.0 equiv) in DCM (20 mL) was added TFA (17.5 g, 43.9 equiv). The reaction was stirred at 25° C. for 2 hours. The mixture was poured into sat. NaHCO₃ aqueous solution (30 mL) and then extracted with DCM (3×20 mL). The combined organic layers were washed with brine (50 mL), dried over sodium sulfate, filtered and concentrated under vacuum to afford the title compound (1.20 g, 89% yield) as white solid; LCMS (ESI, M+1): m/z=388.0.

Step C. 5-(7-(8-ethyl-7-fluoro-3-(methoxymethoxy)naphthalen-1-yl)-2-(methylthio)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide: A mixture of N,N-dimethyl-5-(2-(methylthio)-5,6,7,8-tetrahydropyrido[3,4-d]pyrimidin-4-yl)-5,6,7,8-tetrahydro-4H-pyrazolo[1,5-a][1,4]diazepine-2-carboxamide (1.20 g, 1.0 equiv) and [8-ethyl-7-fluoro-3-(methoxymethoxy)-1-naphthyl]trifluoromethanesulfonate (1.66 g, 1.4 equiv) in toluene (10 mL) were added Pd₂(dba)₃ (284 mg, 0.1 equiv), Cs₂CO₃ (3.03 g, 3.0 equiv) and dicyclohexyl-[2-(2,6-diisopropoxyphenyl)phenyl]phosphane (289 mg, 0.2 equiv). The reaction was degassed and purged with nitrogen 3 times. The reaction was stirred at 90° C. for 5 hours. The mixture was poured into ice-water (w/w=1/1, 20 mL) and extracted with ethyl acetate (3×30 mL). The combined organic layers were washed with brine (35 mL), dried over sodium sulfate, filtered, concentrated under vacuum and purified by flash silica gel chromatography (ISCO®; 20 g SepaFlash® Silica Flash Column, Eluent of 0˜100% Ethylacetate/Petroleum ether gradient and 60˜70% Ethylacetate (10% MeOH)/Petroleum ether gradient @100 mL/min) to afford the title compound (630 mg, 33% yield) as white solid; LCMS (ESI, M+1): m/z=620.3.

Step D. 5-(7-(8-ethyl-7-fluoro-3-(methoxymethoxy)naphthalen-1-yl)-2-(methylsulfinyl)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide: To a solution of 5-(7-(8-ethyl-7-fluoro-3-(methoxymethoxy)naphthalen-1-yl)-2-(methylthio)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide (630 mg, 1.0 equiv) in DCM (7 mL) was added m-CPBA (206 mg, 85% purity, 1.0 equiv) at 0° C. The reaction was stirred at 25° C. for 2 hours. The mixture was filtered and purified by prep-HPLC (column: Phenomenex luna C18 150*25 mm*10 μm; mobile phase: [water (FA)-ACN]; B %: 42%-72%, 10 min) to afford the title compound (250 mg, 39% yield) as white solid; LCMS (ESI, M+1): m/z=636.3.

Step E. 5-(2-(((3S,7aR)-3-((N,N-dimethylsulfamoyl)amino)methyl)hexahydro-1H-pyrrolizin-7a-yl)methoxy)-7-(8-ethyl-7-fluoro-3-(methoxymethoxy)naphthalen-1-yl)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide: To a mixture of 5-(7-(8-ethyl-7-fluoro-3-(methoxymethoxy)naphthalen-1-yl)-2-(methylsulfinyl)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide (60.0 mg, 1.0 equiv) and (3S,8R)-3-[(dimethylsulfamoylamino)methyl]-8-(hydroxymethyl)-1,2,3,5,6,7-hexahydropyrrolizine (30.5 mg, 1.0 equiv, FA salt) in THF (1 mL) was added t-BuONa (27.2 mg, 3.0 equiv) at 0° C. The reaction was stirred at 25° C. for 2 hours. The mixture was concentrated and purified by prep-HPLC (column: Waters Xbridge 150*25 mm*5 μm; mobile phase: [water (NH₄HCO₃)-ACN]; B %: 61%-91%, 8 min) to afford the title compound (30.0 mg, 37% yield) as a white solid; LCMS (ESI, M+1): m/z=849.5.

Step F. 5-(2-(((3S,7aR)-3-(((N,N-dimethylsulfamoyl)amino)methyll)hexahydro-1H-pyrrolizin-7a-yl)methoxy)-7-(8-ethyl-7-fluoro-3-hydroxynaphthalen-1-yl)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide: A mixture of 5-(2-(((3S,7aR)-3-(((N,N-dimethylsulfamoyl)amino)methyl)hexahydro-1H-pyrrolizin-7a-yl)methoxy)-7-(8-ethyl-7-fluoro-3-(methoxymethoxy)naphthalen-1-yl)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide (10.0 mg, 1.0 equiv) in HCl•MeOH (4 M, 0.5 mL) was stirred at 0° C. for 1 hour. The mixture was concentrated and purified by prep-HPLC (column: Phenomenex luna C18 150*25 mm*10 μm; mobile phase: [water (FA)-ACN]; B %: 18%-48%, 10 min) to afford the title compound (8.50 mg) as white solid. 1H NMR (400 MHz, METHANOL-d₄) δ=7.53 (dd, J=5.6, 8.8 Hz, 1H), 7.21-7.11 (m, 1H), 6.99 (s, 2H), 6.66-6.59 (m, 1H), 5.07-4.94 (m, 2H), 4.65-4.53 (m, 2H), 4.32-4.15 (m, 3H), 4.14-4.01 (m, 2H), 3.78-3.66 (m, 1H), 3.62-3.49 (m, 1H), 3.46-3.38 (m, 2H), 3.38-3.35 (m, 3H), 3.30-3.13 (m, 6H), 3.10 (s, 3H), 2.86-2.74 (m, 7H), 1.80 (br s, 6H), 1.20-1.04 (m, 3H); ¹⁹F NMR (376 MHz, METHANOL-d₄) δ=−76.91 (br s, 1F), -122.89 (br s, 1F); LCMS (ESI, M+1): m/z=805.6.

### Example 594

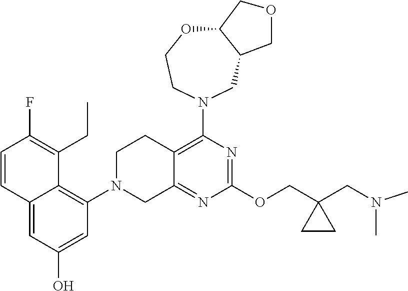 [SMILES: CCc1c(F)ccc2cc(O)cc(N3CCc4c(nc(OCC5(CN(C)C)CC5)nc4N4CCO[C@@H]5COC[C@@H]5C4)C3)c12]

### 4-(2-((1-((dimethylamino)methyl)cyclopropyl)methoxy)-4-((5aS,8aS)-hexahydrofuro[3,4-f][1,4]oxazepin-4(5H)-yl)-5,8-dihydropyrido[3,4-d]pyrimidin-7(6H)-yl)-5-ethyl-6-fluoronaphthalen-2-ol

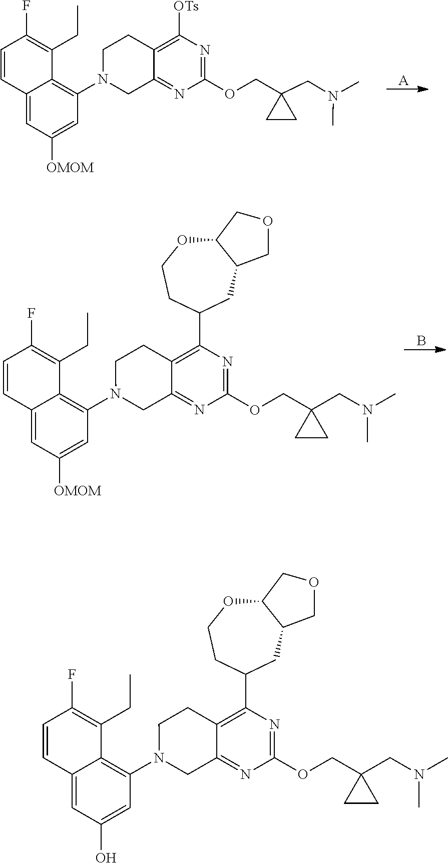

Step A: 1-(1-(((7-(8-ethyl-7-fluoro-3-(methoxymethoxy)naphthalen-1-yl)-4-((5aS,8aS)-hexahydrofuro[3,4-f][1,4]oxazepin-4(5H)-yl)-5,6,7,8-tetrahydropyrido[3,4-d] pyrimidin-2-yl)oxy)methyl)cyclopropyl)-N,N-dimethylmethanamine: To a solution of 2-((1-((dimethylamino)methyl)cyclopropyl)methoxy)-7-(8-ethyl-7-fluoro-3-(methoxymethoxy)naphthalen-1-yl)-5,6,7,8-tetrahydropyrido[3,4-d]pyrimidin-4-yl4-methylbenzenesulfonate (10.0 mg, 1.0 equiv) and (5aS,8aS)-octahydrofuro[3,4-f][1,4]oxazepine (4.31 mg, 2.0 equiv) in DMF (2 mL) were added DIEA (5.83 mg, 3.0 equiv) and 4 Å molecular sieve (1.94 mg, 2.58 equiv). The reaction was stirred at 40° C. for 12 hours. The mixture was diluted with water (2 mL) and extracted with EtOAc (3×2 mL). Combined organic phase was washed with brine, dried, filtered and concentrated to afford the title compound (9.0 mg, 94% yield) as yellow oil; LCMS (ESI, M+1): m/z=636.4.

Step B: 4-(2-((1-((dimethylamino)methyl)cyclopropyl)methoxy)-4-((5aS,8aS)-hexahydrofuro[3,4-f][1,4]oxazepin-4(5H)-yl)-5,8-dihydropyrido[3,4-d]pyrimidin-7(6H)-yl)-5-ethyl-6-fluoronaphthalen-2-ol: A solution of 1-(1-(((7-(8-ethyl-7-fluoro-3-(methoxymethoxy)naphthalen-1-yl)-4-((5aS,8aS)-hexahydrofuro[3,4-f][1,4]oxazepin-4(5H)-yl)-5,6,7,8-tetrahydropyrido[3,4-d]pyrimidin-2-yl)oxy)methyl)cyclopropyl)-N,N-dimethylmethanamine (300 mg, 1.0 equiv) in DMF or methanol was added HCl•MeOH (4 M, 10 equiv). The reaction was stirred at 25° C. for 0.5 hours. The mixture was adjusted to pH=8 with solid NaHCO₃, filtered, concentrated and purified by prep-HPLC (column: Phenomenex Luna C18 150×25 mm×10 μm; mobile phase: [water (FA)-ACN]B %: 14%-44%, 12 min) to afford the title compound (62.2 mg, FA salt) as orange solid. ¹H NMR (400 MHz, CD30D) δ=7.58-7.50 (m, 1H), 7.16 (m, 1H) 7.04-6.93 (m, 2H), 4.50-4.43 (m, 2H), 4.29-4.21 (m, 2H), 4.18-4.14 (m, 2H), 4.14-4.09 (m, 1H), 4.06-4.01 (m, 1H), 4.00-3.96 (m, 1H), 3.92-3.86 (m, 1H), 3.81-3.74 (m, 1H), 3.72-3.66 (m, 1H), 3.62-3.47 (m, 3H), 3.46-3.37 (m, 3H), 3.29-3.14 (m, 2H), 3.08-2.85 (m, 3H), 2.76-2.64 (m, 6H), 2.58-2.54 (m, 1H), 1.13-1.09 (m, 3H), 0.82 (br s, 2H), 0.71 (br s, 2H); LCMS (ESI, M+1): m/z=592.3.

Example 595 to 606 were synthesized according to Example 594.

### Example 595

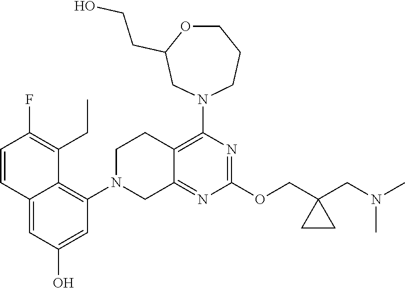 [SMILES: CCc1c(F)ccc2cc(O)cc(N3CCc4c(nc(OCC5(CN(C)C)CC5)nc4N4CCCOC(CCO)C4)C3)c12]

### 4-(2-((1-((dimethylamino)methyl)cyclopropyl)methoxy)-4-(2-(2-hydroxyethyl)-1,4-oxazepan-4-yl)-5,8-dihydropyrido[3,4-d]pyrimidin-7(6H)-yl)-5-ethyl-6-fluoronaphthalen-2-ol

The title compound was obtained as brown solid. ¹H NMR (400 MHz, DMSO-d₆) δ=9.68 (br dd, J=2.0, 4.5 Hz, 1H), 7.59 (br d, J=3.6 Hz, 1H), 7.29-7.17 (m, 1H), 6.98 (br d, J=7.2 Hz, 2H), 4.43 (br d, J=4.4 Hz, 1H), 4.35-4.20 (m, 1H), 4.15-3.77 (m, 7H), 3.72-3.44 (m, 5H), 3.17-2.92 (m, 3H), 2.75-2.54 (m, 2H), 2.27-1.99 (m, 10H), 1.88-1.76 (m, 1H), 1.58 (br d, J=5.6 Hz, 2H), 1.04 (br d, J=6.8 Hz, 3H), 0.55 (br s, 2H), 0.34 (br s, 2H); LCMS (ESI, M+1): m/z=594.4.

### Example 596

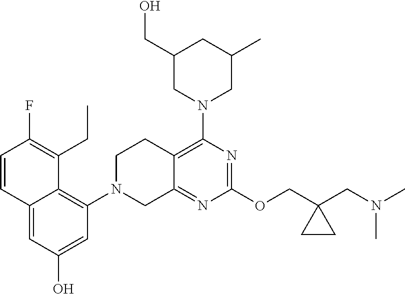 [SMILES: CCc1c(F)ccc2cc(O)cc(N3CCc4c(nc(OCC5(CN(C)C)CC5)nc4N4CC(C)CC(CO)C4)C3)c12]

### 4-(2-((1-((dimethylamino)methyl)cyclopropyl)methoxy)-4-(3-(hydroxymethyl)-5-methylpiperidin-1-yl)-5, 8-dihydropyrido[3,4-d]pyrimidin-7(6H)-yl)-5-ethyl-6-fluoronaphthalen-2-ol

The title compound was obtained as yellow solid (FA salt). ¹HNMR (400 MHz, METHANOL-d₄) δ=0.70-0.77 (m, 2H), 0.82-0.87 (m, 2H), 0.94-1.03 (m, 3H), 1.08-1.15 (m, 3H), 1.38-1.57 (m, 1H), 1.60-1.81 (m, 1H), 1.82-1.96 (m, 1H), 1.97-2.16 (m, 1H), 2.38-2.71 (m, 2H), 2.78 (s, 6H), 3.00-3.11 (m, 2H), 3.12-3.27 (m, 2H), 3.34-3.55 (m, 5H), 3.55-3.70 (m, 2H), 3.71-3.86 (m, 1H), 4.00-4.12 (m, 1H), 4.13-4.48 (m, 3H), 6.94-7.02 (m, 2H), 7.15 (t, J=9.6 Hz, 1H), 7.52 (dd, J=8.8, 6.0 Hz, 1H), 8.53 (s, 1H); LCMS (ESI, M+1): m/z=578.4.

### Example 597

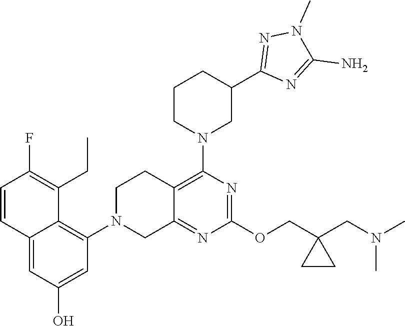 [SMILES: CCc1c(F)ccc2cc(O)cc(N3CCc4c(nc(OCC5(CN(C)C)CC5)nc4N4CCCC(c5nc(N)n(C)n5)C4)C3)c12]

### 4-(4-(3-(5-amino-i-methyl-1H-1,2,4-triazol-3-yl)piperidin-1-yl)-2-((1-((dimethylamino)methyl)cyclopropyl)methoxy)-5,8-dihydropyrido[3,4-d]pyrimidin-7(6H)-yl)-5-ethyl-6-fluoronaphthalen-2-ol

The title compound was obtained as brown solid; ¹H NMR (400 MHz, DMSO-d₆) δ=9.68 (br d, J=1.6 Hz, 1H), 7.59 (dd, J=6.0, 9.0 Hz, 1H), 7.24 (t, J=9.6 Hz, 1H), 6.98 (s, 2H), 6.05 (br d, J=3.2 Hz, 1H), 4.18 (br d, J=12.6 Hz, 1H), 4.08-3.92 (m, 4H), 3.62 (br dd, J=10.8, 17.2 Hz, 1H), 3.44 (d, J=3.2 Hz, 3H), 3.30 (s, 3H), 3.15-2.90 (m, 4H), 2.87-2.58 (m, 3H), 2.17 (br t, J=3.4 Hz, 2H), 2.13 (s, 6H), 1.77 (br s, 3H), 1.05 (dt, J=2.8, 7.2 Hz, 3H), 0.55 (br s, 2H), 0.34 (s, 2H); LCMS (ESI, M+1): m/z=630.4.

### Example 598

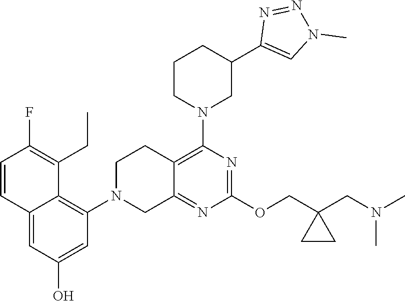 [SMILES: CCc1c(F)ccc2cc(O)cc(N3CCc4c(nc(OCC5(CN(C)C)CC5)nc4N4CCCC(c5cn(C)nn5)C4)C3)c12]

### 4-(2-((1-((dimethylamino)methyl)cyclopropyl)methoxy)-4-(3-(1-methyl-1H-1,2,3-triazol-4-yl)piperidin-1-yl)-5, 8-dihydropyrido[3,4-d]pyrimidin-7(6H)-yl)-5-ethyl-6-fluoronaphthalen-2-ol

The title compound was obtained as white solid. ¹HNMR (400 MHz, METHANOL-d₄) δ=7.68 (d, J=10.4 Hz, 1H), 7.39 (dd, J=5.6, 8.8 Hz, 1H), 7.02 (t, J=9.2 Hz, 1H), 6.91-6.86 (m, 1H), 6.85 (s, 1H), 4.21 (br d, J=12.4 Hz, 1H), 4.15-4.04 (m, 2H), 3.97 (d, J=2.0 Hz, 4H), 3.53 (br t, J=17.2 Hz, 1H), 3.41-3.22 (m, 3H), 3.18-3.10 (m, 1H), 3.08-2.79 (m, 4H), 2.60-2.45 (m, 1H), 2.36-2.23 (m, 2H), 2.15 (d, J=2.0 Hz, 6H), 2.11-2.03 (m, 1H), 1.95-1.40 (m, 4H), 1.20-1.10 (m, 2H), 1.06-0.90 (m, 3H), 0.65-0.47 (m, 2H), 0.36 (s, 2H); ¹⁹F NMR (376 MHz, METHANOL-d₄) 6=-123.02 (br s, 1F); LCMS (ESI, M+1): m/z=615.5.

### Example 599

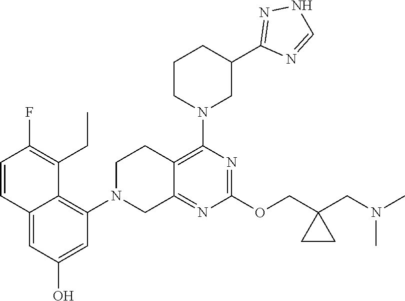 [SMILES: CCc1c(F)ccc2cc(O)cc(N3CCc4c(nc(OCC5(CN(C)C)CC5)nc4N4CCCC(c5nc[nH]n5)C4)C3)c12]

### 4-(4-(3-(1H-1,2,4-triazol-3-yl)piperidin-1-yl)-2-((1-((dimethylamino)methyl)cyclopropyl)methoxy)-5,8-dihydropyrido[3,4-d]pyrimidin-7(6H)-yl)-5-ethyl-6-fluoronaphthalen-2-ol

The title compound was obtained as a yellow solid; ¹H NMR (400 MHz, METHANOL-d₄) δ=8.21 (s, 1H), 7.50 (dd, J=6.0, 8.8 Hz, 1H), 7.14 (t, J=9.2 Hz, 1H), 7.02-6.91 (m, 2H), 4.46-4.31 (m, 1H), 4.28-4.02 (m, 4H), 3.65 (br t, J=17.6 Hz, 1H), 3.53-3.35 (m, 3H), 3.28-2.95 (m, 5H), 2.65 (br d, J=11.6 Hz, 1H), 2.42 (s, 2H), 2.28 (s, 6H), 2.22 (br s, 1H), 1.97-1.89 (m, 2H), 1.81-1.45 (m, 1H), 1.10 (br t, J=7.2 Hz, 3H), 0.66 (br s, 2H), 0.47 (s, 2H); CMS (ESI, M+1): m/z=601.3.

### Example 600

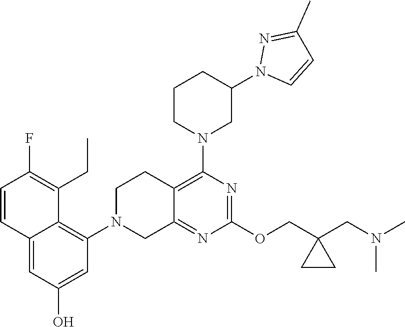 [SMILES: CCc1c(F)ccc2cc(O)cc(N3CCc4c(nc(OCC5(CN(C)C)CC5)nc4N4CCCC(n5ccc(C)n5)C4)C3)c12]

### 4-(2-((1-((dimethylamino)methyl)cyclopropyl)methoxy)-4-(3-(3-methyl-1H-pyrazol-1-yl)piperidin-1-yl)-5, 8-dihydropyrido[3,4-d]pyrimidin-7(6H)-yl)-5-ethyl-6-fluoronaphthalen-2-ol

The title compound was obtained as a white solid (FA salt). ¹H NMR (400 MHz, METHANOL-d₄) δ=8.56 (br s, 1H), 7.63 (d, J=2.0 Hz, 1H), 7.53 (dd, J=6.0, 9.2 Hz, 1H), 7.16 (t, J=9.2 Hz, 1H), 7.06-6.96 (m, 2H), 6.11 (s, 1H), 4.76-4.49 (m, 1H), 4.46-4.29 (m, 2H), 4.26-4.21 (m, 1H), 4.20-4.04 (m, 2H), 3.68 (br dd, J=10.4, 17.2 Hz, 1H), 3.58-3.35 (m, 4H), 3.31-3.25 (m, 1H), 3.19 (br d, J=9.2 Hz, 2H), 3.07-2.66 (m, 4H), 2.65-2.48 (m, 6H), 2.35-2.23 (m, 4H), 2.22-2.09 (m, 1H), 2.06-1.62 (m, 2H), 1.12 (q, J=7.2 Hz, 3H), 0.78 (br d, J=8.4 Hz, 2H), 0.64 (br d, J=7.2 Hz, 2H); ¹⁹F NMR (376 MHz, METHANOL-d₄) 6=-123.05 (br d, 1F); LCMS (ESI, M+1): m/z=614.5.

### Example 601

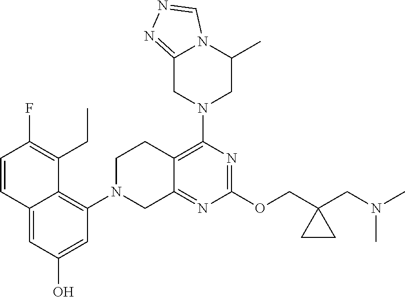 [SMILES: CCc1c(F)ccc2cc(O)cc(N3CCc4c(nc(OCC5(CN(C)C)CC5)nc4N4Cc5nncn5C(C)C4)C3)c12]

### 4-(2-((1-((dimethylamino)methyl)cyclopropyl)methoxy)-4-(5-methyl-5,6-dihydro-[1,2,4]triazolo[4,3-a]pyrazin-7(8H)-yl)-5,8-dihydropyrido[3,4-d]pyrimidin-7(6H)-yl)-5-ethyl-6-fluoronaphthalen-2-ol

The title compound was obtained as yellow solid; ¹H NMR (400 MHz, METHANOL-d₄) δ=8.65 (d, J=17.6 Hz, 1H), 7.51 (dd, J=6.0, 8.8 Hz, 1H), 7.15 (t, J=9.6 Hz, 1H), 7.06-6.88 (m, 2H), 5.16-4.99 (m, 1H), 4.91 (br d, J=16.8 Hz, 1H), 4.78-4.66 (m, 1H), 4.49-4.29 (m, 1H), 4.24-4.15 (m, 2H), 4.15-3.92 (m, 2H), 3.84-3.70 (m, 1H), 3.68-3.45 (m, 2H), 3.41-3.37 (m, 1H), 3.28-3.07 (m, 2H), 2.77 (br dd, J=8.8, 14.8 Hz, 1H), 2.44-2.35 (m, 2H), 2.28 (s, 6H), 1.60 (dd, J=6.8, 10.0 Hz, 3H), 1.12 (t, J=7.2 Hz, 3H), 0.69-0.58 (m, 2H), 0.52-0.40 (m, 2H); LCMS (ESI, M+1): m/z=587.8.

### Example 602

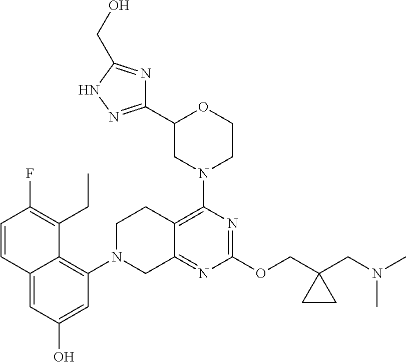 [SMILES: CCc1c(F)ccc2cc(O)cc(N3CCc4c(nc(OCC5(CN(C)C)CC5)nc4N4CCOC(c5n[nH]c(CO)n5)C4)C3)c12]

### 4-(2-((1-((dimethylamino)methyl)cyclopropyl)methoxy)-4-(2-(5-(hydroxymethyl)-1H-1,2,4-triazol-3-yl)morpholino)-5,8-dihydropyrido[3,4-d]pyrimidin-7(6H)-yl)-5-ethyl-6-fluoronaphthalen-2-ol

The title compound was obtained as brown solid (FA salt). ¹H NMR (400 MHz, CD30D) δ=8.53 (s, 1H), 7.52 (dd, J=5.6, 8.8 Hz, 1H), 7.15 (t, J=9.2 Hz, 1H), 6.99 (br d, J=8.4 Hz, 2H), 4.99-4.94 (m, 1H), 4.71 (s, 3H), 4.55-4.19 (m, 4H), 4.12 (br d, J=13.2 Hz, 2H), 4.07-3.78 (m, 2H), 3.70 (br dd, J=10.8, 17.6 Hz, 1H), 3.56-3.50 (m, 1H), 3.48-3.38 (m, 2H), 3.26-3.09 (m, 2H), 3.00 (br s, 2H), 2.74 (br s, 7H), 1.10 (dt, J=3.2, 7.2 Hz, 3H), 0.84 (br d, J=2.8 Hz, 2H), 0.71 (br s, 2H); LCMS (ESI, M+1): m/z=633.5.

### Example 603

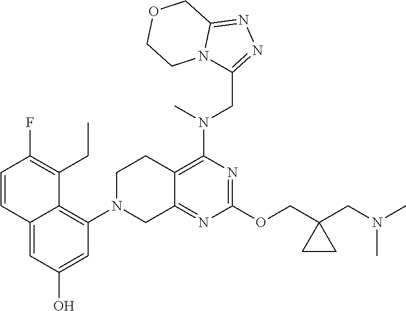 [SMILES: CCc1c(F)ccc2cc(O)cc(N3CCc4c(nc(OCC5(CN(C)C)CC5)nc4N(C)Cc4nnc5n4CCOC5)C3)c12]

### 4-(4-(((5,6-dihydro-8H-[1,2,4]triazolo[3,4-c][1,4]oxazin-3-yl)methyl)(methyl)amino)-2-((1-((dimethylamino)methyl)cyclopropyl)methoxy)-5,8-dihydropyrido[3,4-d]pyrimidin-7(6H)-yl)-5-ethyl-6-fluoronaphthalen-2-ol

The title compound was obtained as brown solid (FA salt). ¹H NMR (400 MHz, METHANOL-d₄) δ=7.52 (dd, J=6.0, 9.2 Hz, 1H), 7.15 (t, J=9.2 Hz, 1H), 7.01-6.93 (m, 2H), 5.07 (d, J=16.0 Hz, 1H), 4.94 (s, 2H), 4.83-4.77 (m, 2H), 4.16 (br s, 7H), 3.68 (br d, J=17.6 Hz, 1H), 3.55-3.48 (m, 1H), 3.45-3.36 (m, 2H), 3.34 (s, 3H), 3.28-3.07 (m, 2H), 2.97 (br d, J=1.6 Hz, 1H), 2.89 (br d, J=14.8 Hz, 1H), 2.72 (br s, 6H), 1.11 (t, J=7.2 Hz, 3H), 0.78 (br s, 2H), 0.69 (br s, 2H); LCMS (ESI, M+1): m/z=617.6.

### Example 604

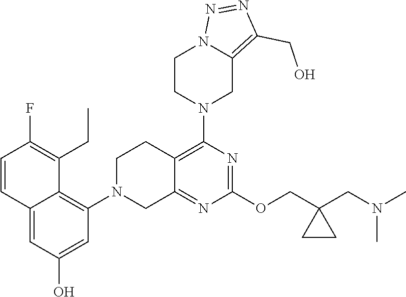 [SMILES: CCc1c(F)ccc2cc(O)cc(N3CCc4c(nc(OCC5(CN(C)C)CC5)nc4N4CCn5nnc(CO)c5C4)C3)c12]

### 4-(2-((1-((dimethylamino)methyl)cyclopropyl)methoxy)-4-(3-(hydroxymethyl)-6,7-dihydro-[1,2,3]triazolo[1,5-a]pyrazin-5(4H)-yl)-5,8-dihydropyrido[3,4-d]pyrimidin-7(6H)-yl)-5-ethyl-6-fluoronaphthalen-2-ol

The title compound was obtained as pink solid (FA salt). ¹H NMR (400 MHz, METHANOL-d₄) δ=7.52 (dd, J=6.0, 8.8 Hz, 1H), 7.15 (t, J=9.2 Hz, 1H), 7.02-6.94 (m, 2H), 5.05-4.90 (m, 2H), 4.65 (ddd, J=4.4, 8.4, 12.8 Hz, 2H), 4.59-4.44 (m, 2H), 4,38-4.29 (m, 1H), 4.28-4.21 (m, 2H), 4.13 (br d, J=18.0 Hz, 1H), 3.94-3.82 (m, 1H), 3.73 (br d, J=18.0 Hz, 1H), 3.60-3.50 (m, 1H), 3.46-3.34 (m, 2H), 3.27-3.13 (m, 3H), 3.04-2.86 (m, 2H), 2.83-2.76 (m, 1H), 2.72 (br s, 5H), 1.19-1.02 (m, 3H), 0.83 (s, 2H), 0.72 (s, 2H); LCMS (ESI, M+1): m/z=603.5.

### Example 605

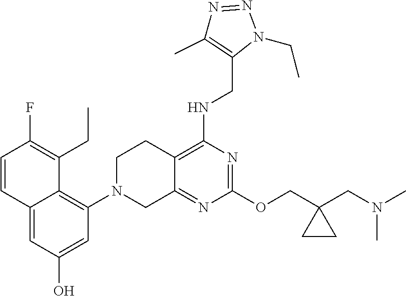 [SMILES: CCc1c(F)ccc2cc(O)cc(N3CCc4c(nc(OCC5(CN(C)C)CC5)nc4NCc4c(C)nnn4CC)C3)c12]

### 4-(2-((1-((dimethylamino)methyl)cyclopropyl)methoxy)-4-(((1-ethyl-4-methyl-1H-1,2,3-triazol-5-yl)methyl)amino)-5,8-dihydropyrido[3,4-d]pyrimidin-7(6H)-yl)-5-ethyl-6-fluoronaphthalen-2-ol

The title compound was obtained as brown solid (FA salt). ¹H NMR (400 MHz, METHANOL-d₄) δ=8.55 (br s, 1H), 7.51 (dd, J=6.0, 8.8 Hz, 1H), 7.14 (t, J=9.6 Hz, 1H), 7.05-6.92 (m, 2H), 4.92-4.89 (m, 2H), 4.81-4.79 (m, 3H), 4.46 (q, J=6.8 Hz, 2H), 4.19-4.10 (m, 2H), 3.95-3.85 (m, 1H), 3.69 (br d, J=17.2 Hz, 1H), 3.59-3.46 (m, 1H), 2.90-2.70 (m, 3H), 2.69-2.42 (m, 7H), 2.33 (s, 3H), 1.46 (t, J=7.2 Hz, 3H), 1.03 (t, J=7.3 Hz, 3H), 0.74 (br s, 2H), 0.63 (br d, J=4.8 Hz, 2H); LCMS (ESI, M+1): m/z=589.5.

### Example 606

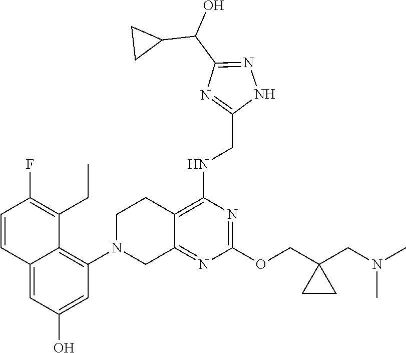 [SMILES: CCc1c(F)ccc2cc(O)cc(N3CCc4c(nc(OCC5(CN(C)C)CC5)nc4NCc4nc(C(O)C5CC5)n[nH]4)C3)c12]

### 4-(4-(((3-(cyclopropyl(hydroxy)methyl)-1H-1,2,4-triazol-5-yl)methyl)amino)-2-((1-((dimethylamino)methyl)cyclopropyl)methoxy)-5,8-dihydropyrido[3,4-d]pyrimidin-7(6H)-yl)-5-ethyl-6-fluoronaphthalen-2-ol

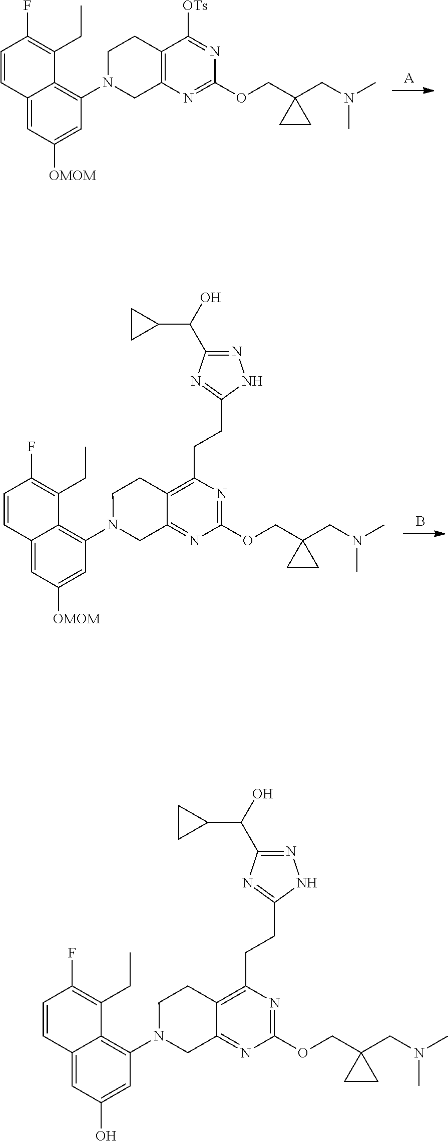

The title compound was obtained as brown solid (FA salt). ¹H NMR (400 MHz, METHANOL-d₄) δ=7.51 (dd, J=6.0, 9.0 Hz, 1H), 7.18-7.08 (m, 1H), 7.02 (d, J=2.4 Hz, 1H), 6.97 (d, J=2.4 Hz, 1H), 4.84-4.69 (m, 5H), 4.26-4.13 (m, 2H), 4.12-4.04 (m, 1H), 3.90 (br d, J=16.8 Hz, 1H), 3.67 (br d, J=16.4 Hz, 1H), 3.61-3.52 (m, 1H), 2.96-2.79 (m, 3H), 2.77-2.56 (m, 7H), 1.33 (br d, J=2.8 Hz, 1H), 1.05 (t, J=7.2 Hz, 3H), 0.81-0.73 (m, 2H), 0.68-0.59 (m, 3H), 0.58-0.48 (m, 2H), 0.41 (br dd, J=4.8, 9.2 Hz, 1H); LCMS (ESI, M+1): m/z=617.6.

### Example 607

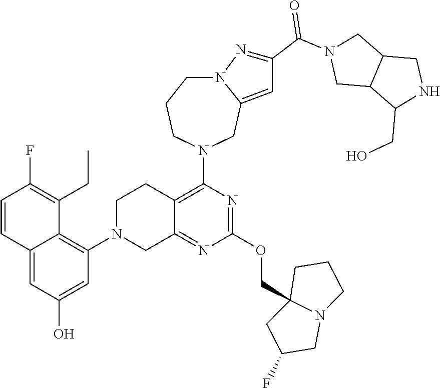 [SMILES: CCc1c(F)ccc2cc(O)cc(N3CCc4c(nc(OC[C@@]56CCCN5C[C@H](F)C6)nc4N4CCCn5nc(C(=O)N6CC7CNC(CO)C7C6)cc5C4)C3)c12]

### (5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(4-(hydroxymethyl)hexahydropyrrolo[3,4-c]pyrrol-2(1H)-yl)methanone

Synthesized according to Example 233 except that HCl was used instead of TFA in the last step. The title compound was obtained as FA salt. ¹H NMR (400 MHz, METHANOL-d₄) δ=8.51 (s, 1H), 7.53 (dd, J=5.6, 8.8 Hz, 1H), 7.16 (t, J=9.2 Hz, 1H), 6.99 (s, 2H), 6.75 (br s, 1H), 5.57-5.18 (m, 1H), 5.12-4.97 (m, 1H), 4.67-4.48 (m, 2H), 4.35-4.12 (m, 5H), 4.07 (br d, J=17.6 Hz, 2H), 3.94-3.67 (m, 5H), 3.65-3.37 (m, 8H), 3.30-3.09 (m, 5H), 3.06-2.85 (m, 1H), 2.82-2.68 (m, 1H), 2.52-2.20 (m, 4H), 2.18-1.88 (m, 4H), 1.13 (br t, J=6.8 Hz, 3H); ¹⁹F NMR (377 MHz, METHANOL-d₄) δ=−122.91 (br s, 1F), -173.59 (br s, 1F); LCMS (ESI, M+1). m/z=784.2.

### Example 608

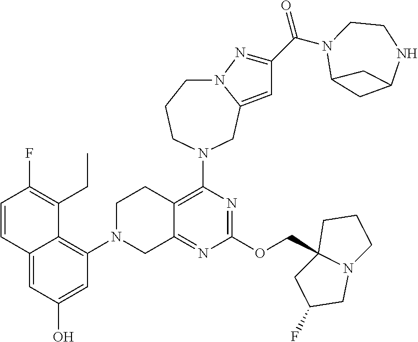 [SMILES: CCc1c(F)ccc2cc(O)cc(N3CCc4c(nc(OC[C@@]56CCCN5C[C@H](F)C6)nc4N4CCCn5nc(C(=O)N6CCNC7CC6C7)cc5C4)C3)c12]

### (2,5-diazabicyclo[4.1.1]octan-2-yl)(5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

Synthesized according to Example 233 except that HCl was used instead of TFA in the last step. The title compound was obtained as FA salt. ¹H NMR (400 MHz, METHANOL-d₄) δ=8.74-8.22 (m, 2H), 7.61-7.47 (m, 1H), 7.24-7.08 (m, 1H), 6.99 (s, 2H), 6.53 (br d, J=3.6 Hz, 1H), 5.61-5.29 (m, 1H), 5.25-5.01 (m, 2H), 4.56 (br s, 3H), 4.39-4.18 (m, 4H), 4.18-3.85 (m, 4H), 3.70 (br d, J=17.6 Hz, 1H), 3.64-3.45 (m, 6H), 3.44-3.35 (m, 2H), 3.26-3.14 (m, 3H), 3.07-2.83 (m, 2H), 2.82-2.68 (m, 1H), 2.54-2.22 (m, 6H), 2.19 (br s, 3H), 2.04-1.93 (m, 1H), 1.20-0.94 (m, 3H); ¹⁹F NMR (376 MHz, METHANOL-d₄) δ=−122.91 (br s, 1F), -173.77 (br d, J=42.9 Hz, 1F); LCMS (ESI, M+1): m/z=754.2.

### Example 609

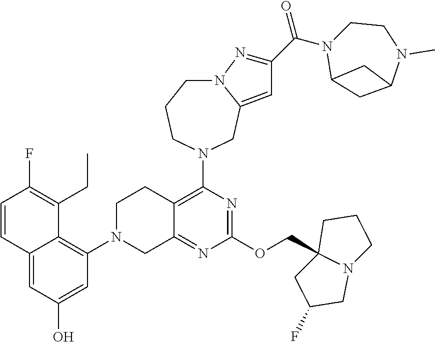 [SMILES: CCc1c(F)ccc2cc(O)cc(N3CCc4c(nc(OC[C@@]56CCCN5C[C@H](F)C6)nc4N4CCCn5nc(C(=O)N6CCN(C)C7CC6C7)cc5C4)C3)c12]

### (5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)(5-methyl-2,5-diazabicyclo[4.1.1]octan-2-yl)methanone

Synthesized according to Example 233 except that HCl was used instead of TFA in the last step. The title compound was obtained as white solid (FA salt). ¹H NMR (400 MHz, METHANOL-d₄) δ=8.49 (br s, 1H), 7.51 (dd, J=5.6, 9.2 Hz, 1H), 7.14 (t, J=9.2 Hz, 1H), 6.97 (s, 2H), 6.66-6.54 (m, 1H), 5.53-5.32 (m, 1H), 5.02-4.92 (m, 3H), 4.60-4.36 (m, 3H), 4.35-4.24 (m, 2H), 4.23-4.13 (m, 2H), 4.11-4.00 (m, 2H), 3.68 (br d, J=17.6 Hz, 1H), 3.64-3.48 (m, 5H), 3.37 (br dd, J=5.2, 7.2 Hz, 2H), 3.27-3.14 (m, 4H), 3.08 (br d, J=1.6 Hz, 1H), 2.68 (br d, J=2.0 Hz, 3H), 2.54-2.46 (m, 3H), 2.44-2.20 (m, 6H), 2.20-2.10 (m, 2H), 2.10-1.97 (m, 2H), 1.09 (br t, J=6.4 Hz, 3H); LCMS (ESI, M+1): m/z=767.4.

### Example 610

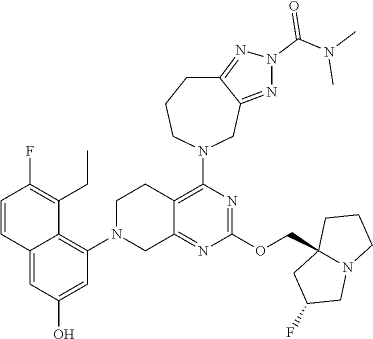 [SMILES: CCc1c(F)ccc2cc(O)cc(N3CCc4c(nc(OC[C@@]56CCCN5C[C@H](F)C6)nc4N4CCCc5nn(C(=O)N(C)C)nc5C4)C3)c12]

### 5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-N,N-dimethyl-5,6,7,8-tetrahydro-[1,2,3]triazolo[4,5-c]azepine-2(4H)-carboxamide

Synthesized according to Example 248. The title compound was obtained as yellow solid. ¹H NMR (400 MHz, METHANOL-d₄) δ=7.50 (dd, J=5.6, 8.8 Hz, 1H), 7.14 (t, J=9.2 Hz, 1H), 7.02-6.93 (m, 2H), 5.36-5.16 (m, 1H), 5.07 (dd, J=6.4, 16.0 Hz, 1H), 4.84-4.78 (m, 1H), 4.68-4.53 (m, 1H), 4.32-4.20 (m, 1H), 4.12-3.96 (m, 4H), 3.70-3.62 (m, 1H), 3.56-3.47 (m, 1H), 3.39-3.33 (m, 2H), 3.27-3.21 (m, 2H), 3.21-3.17 (m, 6H), 3.17-3.12 (m, 2H), 3.04-2.94 (m, 3H), 2.72 (br d, J=13.6 Hz, 1H), 2.32-2.02 (m, 4H), 2.01-1.80 (m, 4H), 1.14-1.04 (m, 3H); LCMS (ESI, M+1): m/z=688.3.

### Example 611

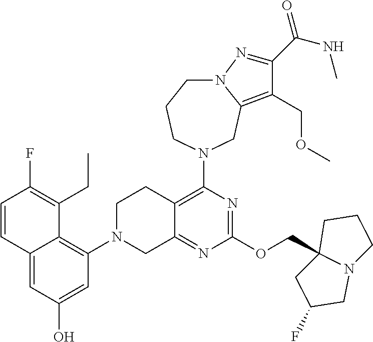 [SMILES: CCc1c(F)ccc2cc(O)cc(N3CCc4c(nc(OC[C@@]56CCCN5C[C@H](F)C6)nc4N4CCCn5nc(C(=O)NC)c(COC)c5C4)C3)c12]

### 5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-3-(methoxymethyl)-N-methyl-5,6,7,8-tetrahydro-4H-pyrazolo[1,5-a][1,4]diazepine-2-carboxamide

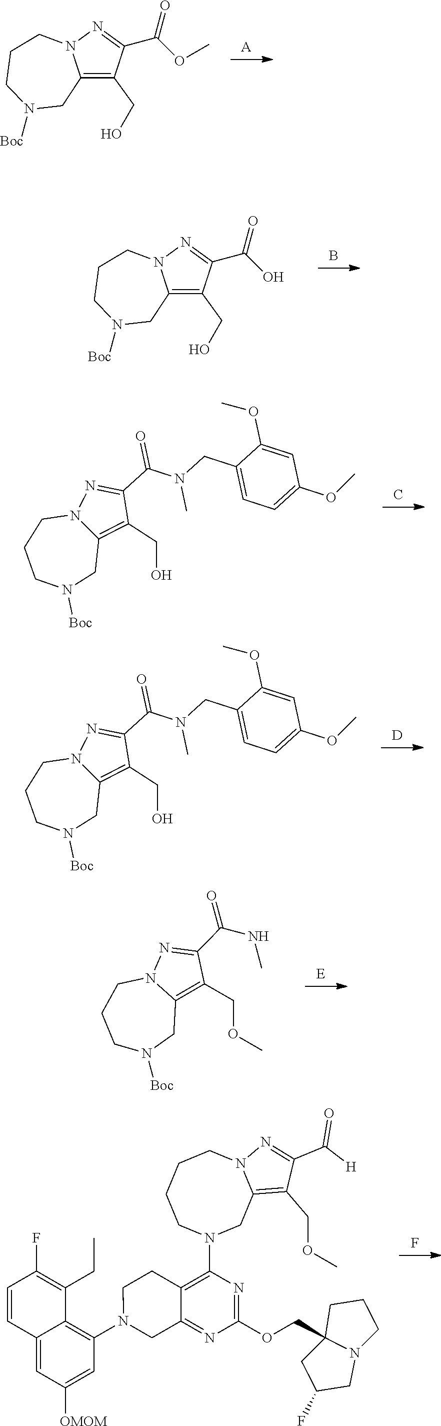

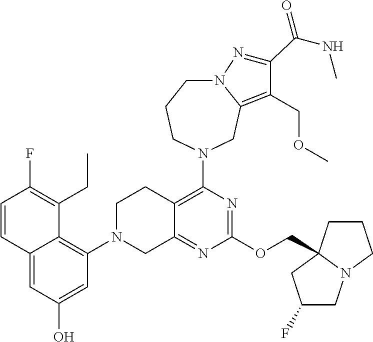 [SMILES: CCc1c(F)ccc2cc(O)cc(N3CCc4c(nc(OC[C@@]56CCCN5C[C@H](F)C6)nc4N4CCCn5nc(C(=O)NC)c(COC)c5C4)C3)c12]

Step A. 5-(tert-butoxycarbonyl)-3-(hydroxymethyl)-5,6,7,8-tetrahydro-4H-pyrazolo[1,5-a][1,4]diazepine-2-carboxylic acid: To a solution of 5-tert-butyl 2-methyl 3-(hydroxymethyl)-7,8-dihydro-4H-pyrazolo[1,5-a][1,4]diazepine-2,5(6H)-dicarboxylate (500 mg, 1.0 equiv) in THF (2 mL) and H₂O (2 mL) was added NaOH (1.23 g, 20 equiv). The reaction was stirred at 25° C. for 2 hours. The mixture was diluted with water (10 mL) and concentrated under vacuum to remove THE (2 mL). The mixture was adjusted to pH=4 with HCl (2 mL, 2 M) and extracted with ethyl acetate (3×15 mL). The combined organic layers were dried over anhydrous sodium sulfate, filtered, concentrated and purified with reversed phase flash [C18, water (FA, 0.1%)/acetonitrile] to afford title compound (450 mg, 90% yield) as yellow solid; LCMS (ESI, M-73): m/z=237.9

Step B. tert-butyl 2-((2,4-dimethoxybenzyl)(methyl)carbamoyl)-3-(hydroxymethyl)-7,8-dihydro-4H-pyrazolo[1,5-alr1,4]diazepine-5(6H)-carboxylate: To a solution of 5-(tert-butoxycarbonyl)-3-(hydroxymethyl)-5,6,7,8-tetrahydro-4H-pyrazolo[1,5-a][1,4]diazepine-2-carboxylic acid (100 mg, 1.0 equiv) and 1-(2,4-dimethoxyphenyl)-N-methylmethanamine (175 mg, 3.0 equiv) in DMF (0.5 mL) were added DIEA (415 mg, 10 equiv) and HATU (366 mg, 3.0 equiv). The reaction was stirred at 25° C. for 1 hour. The mixture was filtered and purified with reversed phase flash [C18, water (FA, 0.1%)/acetonitrile] to afford title compound (120 mg, 78% yield) as yellow oil; LCMS (ESI, M+1): m/z=475.3

Step C. tert-butyl 2-((2,4-dimethoxybenzyl)(methyl)carbamoyl)-3-(methoxymethyl)-7,8-dihydro-4H-pyrazolo[1,5-a][1,4]diazepine-5(6H)-carboxylate: To a mixture of tert-butyl 2-((2,4-dimethoxybenzyl)(methyl)carbamoyl)-3-(hydroxymethyl)-7,8-dihydro-4H-pyrazolo[1,5-a][1,4]diazepine-5(6H)-carboxylate (120 mg, 1.0 equiv) in THE (0.5 mL) was added NaH (101 mg, 10 equiv) at 0° C. The reaction was stirred at 0° C. for 0.5 hr. To the mixture was added Mel (718 mg, 20 equiv). The reaction was stirred at 25° C. for 1 hour. The mixture was quenched with NH₄Cl (5 mL) and extracted with ethyl acetate (3×10 mL). The combined organic layers were dried over anhydrous sodium sulfate, filtered and concentrated to afford title compound (120 mg, crude) as yellow solid and used into next step without further purification; LCMS (ESI, M+1): m/z=489.4.

Step D. 3-(methoxymethyl)-N-methyl-5,6,7,8-tetrahydro-4H-pyrazolo[1,5-a][1,4]diazepine-2-carboxamide: A mixture of tert-butyl 2-((2,4-dimethoxybenzyl)(methyl)carbamoyl)-3-(methoxymethyl)-7,8-dihydro-4H-pyrazolo[1,5-a][1,4]diazepine-5(6H)-carboxylate (100 mg, 1.0 equiv) in TFA (1.54 g, 66 equiv) was stirred at 25° C. for 0.5 hour. The mixture was concentrated to afford title compound (150 mg, crude) as pink solid.

Step E. 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-3-(methoxymethyl)-N-methyl-5,6,7,8-tetrahydro-4H-pyrazolo[1,5-a][1,4]diazepine-2-carboxamide: To a mixture of 7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (200 mg, 1.0 equiv) and 3-(methoxymethyl)-N-methyl-5,6,7,8-tetrahydro-4H-pyrazolo[1,5-a][1,4]diazepine-2-carboxamide (122 mg, 1.2 equiv, TFA) in DMF (1 mL) was added DIEA (742 mg, 20 equiv) and 4 Å molecular sieve (10 mg). The reaction was stirred at 40° C. for 12 hours. The mixture was filtered and purified with reversed phase flash [C18, water (FA, 0.1%)/acetonitrile] to afford title compound (120 mg, 54% yield) as yellow solid; LCMS (ESI, M+1): m/z=761.5.

Step F. 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-3-(methoxymethyl)-N-methyl-5,6,7,8-tetrahydro-4H-pyrazolo[1,5-a][1,4]diazepine-2-carboxamide: A mixture of 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-3-(methoxymethyl)-N-methyl-5,6,7,8-tetrahydro-4H-pyrazolo[1,5-a][1,4]diazepine-2-carboxamide (100 mg, 1.0 equiv) in HCl-MeOH (1 mL, 30 equiv) was stirred at 25° C. for 0.5 hour. The mixture was diluted with water (3 mL) and concentrated under vacuum to remove MeOH (1 mL). The mixture was adjusted to pH=7 with NaHCO₃ (3 mL) and extracted with ethyl acetate (3×5 mL). The combined organic layers were dried over anhydrous sodium sulfate, concentrated, and purified by prep-HPLC (column: Phenomenex C18 150*25 mm*10 μm; mobile phase: [water (NH₄HCO₃)-ACN];B %: 36%-66%,8 min) to afford title compound (58.7 mg, 61% yield) as orange solid; ¹H NMR (400 MHz, METHANOL-d₄) δ=7.51 (dd, J=6.0, 9.2 Hz, 1H), 7.14 (t, J=9.2 Hz, 1H), 6.95 (s, 2H), 5.38-5.16 (m, 1H), 5.00-4.91 (m, 1H), 4.79-4.68 (m, 3H), 4.60-4.38 (m, 3H), 4.14-3.92 (m, 5H), 3.68 (br d, J=17.6 Hz, 1H), 3.50-3.36 (m, 3H), 3.28 (s, 3H), 3.17 (br s, 2H), 3.16-3.11 (m, 2H), 2.97 (dt, J=5.6, 9.2 Hz, 1H), 2.86 (s, 3H), 2.71-2.60 (m, 1H), 2.33-2.10 (m, 4H), 2.09-1.99 (m, 1H), 1.97-1.78 (m, 3H), 1.11 (t, J=7.2 Hz, 3H); LCMS (ESI, M+1): m/z=717.7.

### Example 612

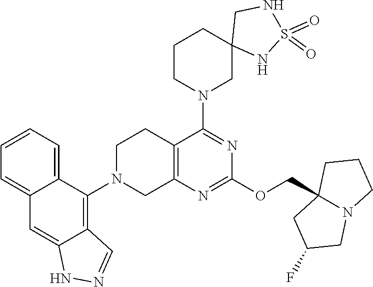 [SMILES: O=S1(=O)NCC2(CCCN(c3nc(OC[C@@]45CCCN4C[C@H](F)C5)nc4c3CCN(c3c5ccccc5cc5[nH]ncc35)C4)C2)N1]

### 7-(7-(1H-benzo[f]indazol-4-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-2-thia-1,3,7-triazaspiro[4.5]decane 2,2-dioxide

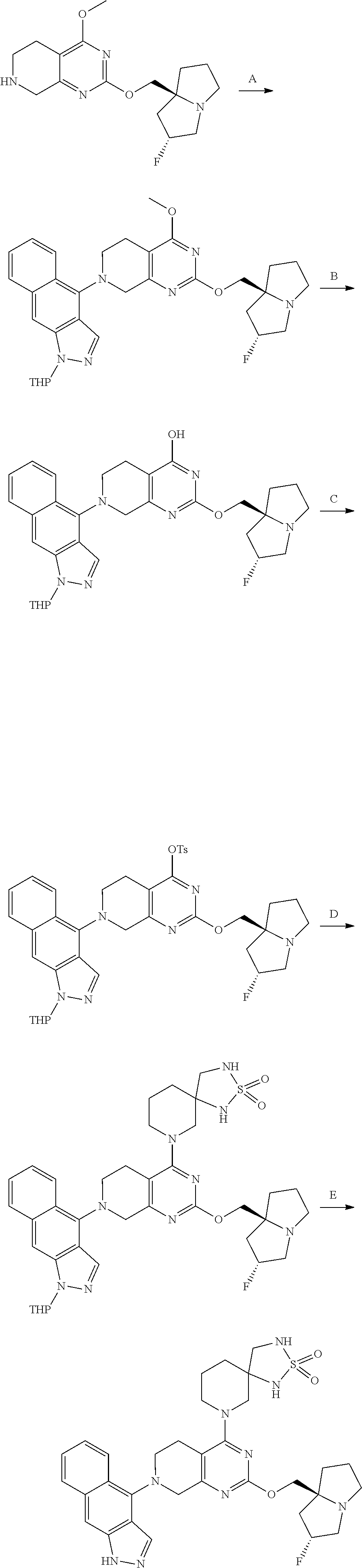

Step A. 4-(2-(((2R,7aS)-2-fluorohexahydro-1H-pyrrolizin-7a-yl)methoxy)-4-methoxy-5,6-dihydropyrido[3,4-d]pyrimidin-7(8H)-yl)-1-(tetrahydro-2H-pyran-2-yl)-1H-benzo[f]indazole: To a mixture of 2-(((2R,7aS)-2-fluorohexahydro-1H-pyrrolizin-7a-yl)methoxy)-4-methoxy-5,6,7,8-tetrahydropyrido[3,4-d]pyrimidine (1.40 g, 1.0 equiv), 4-bromo-1-(tetrahydro-2H-pyran-2-yl)-1H-benzo[f]indazole (1.44 g, 1.0 equiv) and dicyclohexyl-[2-(2,6-diisopropoxyphenyl)phenyl]phosphane (405 mg, 0.2 equiv) in toluene (15 mL) were added Cs₂CO₃ (4.24 g, 3.0 equiv) and Pd₂(dba)₃ (398 mg, 0.1 equiv). The reaction was degassed and purged with nitrogen 3 times. The mixture was stirred at 100° C. for 5 hours. The mixture was diluted with H₂O (20 mL) and extracted with ethyl acetate (2×30 mL). The combined organic layers were washed with brine (20 mL), dried over anhydrous sodium sulfate, concentrated, and purified with reversed phase flash [water (FA 0.1%)/acetonitrile] to afford the title compound (1.4 g, 54% yield) as yellow solid; LCMS (ESI, M+1): m/z=573.6.

Step B. 2-(((2R,7aS)-2-fluorohexahydro-1H-pyrrolizin-7a-yl)methoxy)-7-(1-(tetrahydro-2H-pyran-2-yl)-1H-benzo[f]indazol-4-yl)-5,6,7,8-tetrahydropyrido[3,4-d] pyrimidin-4-ol: To a solution of EtSH (2.69 g, 15.5 equiv) in DMAC (16 mL) was added NaH (335 mg, 60% purity, 3.0 equiv). The reaction was stirred at 0° C. for 0.5 hour. A solution of 4-(2-(((2R,7aS)-2-fluorohexahydro-1H-pyrrolizin-7a-yl)methoxy)-4-methoxy-5,6-dihydropyrido[3,4-d]pyrimidin-7(8H)-yl)-1-(tetrahydro-2H-pyran-2-yl)-1H-benzo[f]indazole (1.60 g, 1.0 equiv) in DMAC (4 mL) was added into above mixture. The reaction was stirred at 60° C. for 0.5 hour. The mixture was diluted with water (30 ml). The mixture was adjusted to pH=5 with 2 N HCl and extracted with ethyl acetate (2×50 mL). The combined organic layer was washed with brine (30 mL), dried over anhydrous sodium sulfate, concentrated to afford the title compound (1.40 g, 87% yield) as yellow solid; LCMS (ESI, M+1): m/z=559.3.

Step C. 2-(((2R,7aS)-2-fluorohexahydro-1H-pyrrolizin-7a-yl)methoxy)-7-(1-(tetrahydro-2H-pyran-2-yl)-1H-benzo[f]indazol-4-yl)-5,6,7,8-tetrahydropyrido[3,4-d]pyrimidin-4-yl 4-methylbenzenesulfonate: To a mixture of 2-(((2R,7aS)-2-fluorohexahydro-1H-pyrrolizin-7a-yl)methoxy)-7-(1-(tetrahydro-2H-pyran-2-yl)-1H-benzo[f]indazol-4-yl)-5,6,7,8-tetrahydropyrido[3,4-d]pyrimidin-4-ol (1.40 g 1.0 equiv), DIEA (972 mg, 3.0 equiv) and DMAP (30.6 mg, 0.1 equiv) in DCM (15 mL) was added 4-methylbenzenesulfonyl chloride (549 mg, 1.15 equiv). The reaction was stirred at 25° C. for 0.5 hours. The mixture was diluted with H₂O (10 mL) and extracted with DCM (2×50 mL). The combined organic layers were washed with brine (20 mL), dried over anhydrous sodium sulfate, concentrated and purified with reversed phase flash [water (FA 0.1%)/acetonitrile] to afford the title compound (1.22 g, 61% yield) as yellow solid; LCMS (ESI, M+1): m/z=713.3.

Step D. 7-(2-(((2R,7aS)-2-fluorohexahydro-1H-pyrrolizin-7a-yl)methoxy)-7-(1-(tetrahydro-2H-pyran-2-yl)-1H-benzo[f]indazol-4-yl)-5,6,7,8-tetrahydropyrido[3,4-d] pyrimidin-4-yl)-2-thia-1,3,7-triazaspiro[4.5]decane 2,2-dioxide: The mixture of 2-(((2R,7aS)-2-fluorohexahydro-1H-pyrrolizin-7a-yl)methoxy)-7-(1-(tetrahydro-2H-pyran-2-yl)-1H-benzo[f]indazol-4-yl)-5,6,7,8-tetrahydropyrido[3,4-d]pyrimidin-4-yl 4-methylbenzenesulfonate (200 mg 1.0 equiv) and 2-thia-1,3,7-triazaspiro[4.5]decane 2,2-dioxide (80.5 mg, 1.5 equiv) in DMF (0.5 mL) were added 4 Å molecular sieve (5 mg) and DIEA (363 mg, 10.0 equiv). The reaction was stirred at 60° C. for 4 hours. The mixture was filtered and purified with reversed phase flash [water (FA 0.1%)/acetonitrile] to afford the title compound (200 mg, 93% yield) as yellow solid; LCMS (ESI, M+1): m/z=732.2.

Step E. 7-(7-(1H-benzo[f]indazol-4-yl)-2-(((2R,7aS)-2-fluorohexahydro-1H-pyrrolizin-7a-yl)methoxy)-5,6,7,8-tetrahydropyrido[3,4-d] pyrimidin-4-yl)-2-thia-1,3,7-triazaspiro[4.5]decane 2,2-dioxide: A mixture of 7-(2-(((2R,7aS)-2-fluorohexahydro-1H-pyrrolizin-7a-yl)methoxy)-7-(1-(tetrahydro-2H-pyran-2-yl)-1H-benzo[f]indazol-4-yl)-5,6,7,8-tetrahydropyrido[3,4-d]pyrimidin-4-yl)-2-thia-1,3,7-triazaspiro[4.5]decane 2,2-dioxide (130 mg, 1.0 equiv) in TFA (2.31 g, 114 equiv) was stirred 20° C. for 0.5 hours. The mixture was concentrated. The residue was diluted with water (2 mL). The mixture was adjusted pH=7 with NaHCO₃ (3 mL) and extracted with DCM (2×10 mL). The combined organic layers were washed with brine (5 mL), dried over anhydrous sodium sulfate, concentrated and purified with prep-HPLC (column: Waters xbridge 150*25 mm 10 μm; mobile phase: [water(NH₄HCO₃)-ACN];B %: 35% -65%,10 min) to afford the title compound (4.68 mg, 4% yield) as yellow solid; ¹H NMR (400 MHz, DMSO-d₆) δ=13.10 (s, 1H), 8.44 (s, 1H), 8.31 (br d, J=8.8 Hz, 1H), 7.97 (br d, J=8.4 Hz, 1H), 7.78 (s, 1H), 7.42 (br t, J=7.2 Hz, 1H), 7.35-7.28 (m, 1H), 7.18-7.11 (m, 2H), 5.35-5.17 (m, 1H), 4.37 (br s, 2H), 4.00-3.95 (m, 1H), 3.91-3.86 (m, 1H), 3.68 (br s, 2H), 3.12-2.96 (m, 6H), 2.87-2.75 (m, 2H), 2.09 (br s, 1H), 2.06-1.92 (m, 3H), 1.87-1.68 (m, 8H), 1.68-1.47 (m, 2H); LCMS (ESI, M+1): m/z=648.1

### Example 613

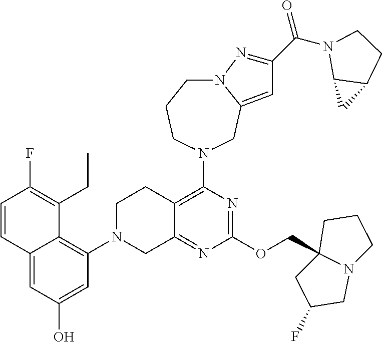 [SMILES: CCc1c(F)ccc2cc(O)cc(N3CCc4c(nc(OC[C@@]56CCCN5C[C@H](F)C6)nc4N4CCCn5nc(C(=O)N6CC[C@@H]7C[C@@H]76)cc5C4)C3)c12]

### ((1S,5R)-2-azabicyclo[3.1.0]hexan-2-yl)(5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

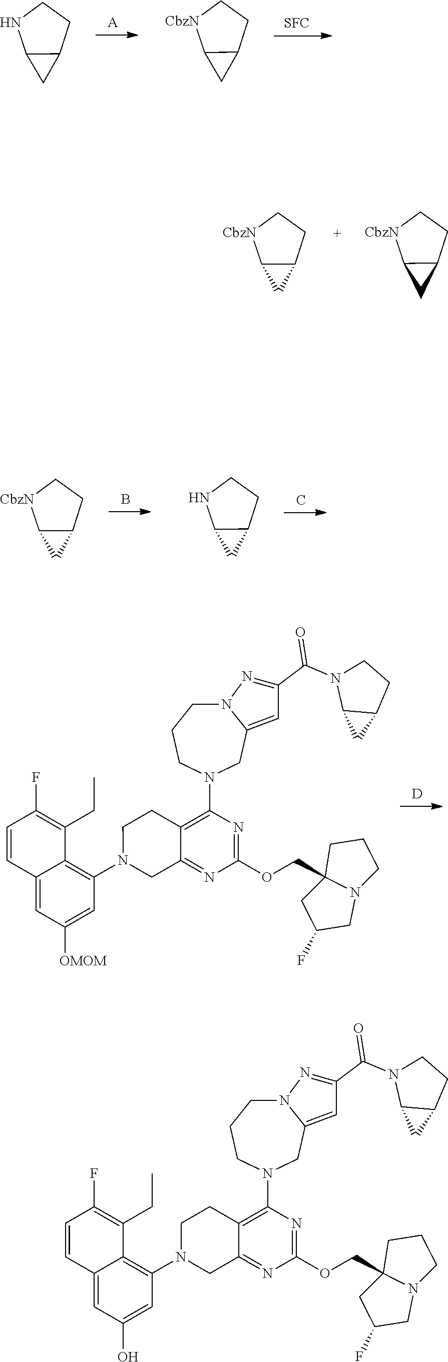

Step A. benzyl 2-azabicyclo[3.1.0]hexane-2-carboxylate: To a solution of 2-azabicyclo[3.1.0]hexane (3.00 g, 1.0 equiv) in DCM (30.0 mL) was added DIEA (14.0 g, 3.0 equiv). The mixture was stirred at 0° C. for 10 minutes. CbzCl (7.39 g, 1.2 equiv) was added and the reaction was stirred at 0° C. for 1 hour. The mixture was diluted with water (40 mL) and extracted with ethyl acetate (2×20 mL). The combined organic layers were washed with brine (20 mL), dried over anhydrous sodium sulfate, concentrated and purified with column chromatography (SiO2, petroleum ether/ethyl acetate 20/1 to 5/1) followed with SFC separation [column: REGIS (S,S) WHELK-01 (250 mm×50 mm,10 μm); A: CO₂; B, IPA(0.1% NH₃H₂O); B %: 35% over 2.8 min)] to afford two isomers. benzyl (1S,5R)-2-azabicyclo[3.1.0]hexane-2-carboxylate (2.3 g, 29% yield) as yellow oil; SFC>99% ee, column: (S,S)Whelk-01 50×4.6 mm I.D., 3 μm, mobile phase 5%-40% MeOH(0.05% DEA) in CO₂, flow rate: 3 mL/min, detector: 220 nm, t_{R}: 1.355 min;¹H NMR (400 MHz, CiLOROFORM-d) δ=7.44-7.28 (m, 5H), 5.31-5.06 (m, 2H), 3.74 (br s, 1H), 3.60-3.40 (m, 1H), 3.04 (br s, 1H), 2.23-2.06 (m, 1H), 1.99-1.89 (m, 1H), 1.62-1.50 (m, 1H), 0.72 (br s, 1H), 0.60-0.52 (m, 1H); LCMS (ESI, M-44): m/z=174.3. benzyl (1R,5S)-2-azabicyclo[3.1.0]hexane-2-carboxylate (3.4 g, 45% yield) as yellow oil; SFC: >99% ee, column: (S,S)Whelk-01 50×4.6 mm I.D., 3 gm, mobile phase 5%-40% MeOH(0.05% DEA) in CO₂, flow rate: 3 mL/min, detector: 220 nm, tR: 1.526 min; ¹H NMR (400 MHz, CiLOROFORM-d) δ=7.47-7.28 (m, 5H), 5.31-5.07 (m, 2H), 3.75 (br s, 1H), 3.62-3.40 (m, 1H), 3.05 (br s, 1H), 2.12 (br s, 1H), 2.02-1.90 (m, 1H), 1.64-1.49 (m, 1H), 0.73 (br s, 1H), 0.63-0.49 (m, 1H).

Step B. (1S,5R)-2-azabicyclo[3.1.0]hexane: To a solution of benzyl (1S,5R)-2-azabicyclo[3.1.0]hexane-2-carboxylate (100 mg, 1.0 equiv) in EtOH (10.0 mL) and HCl•dioxane (0.50 mL) was added Pd/C (10 mg, 10% purity, 1.0 equiv) under N₂ atmosphere. The suspension was degassed and purged with H₂ for 3 times. The reaction was stirred under H₂ (15 Psi) at 20° C. for 2 hours. The mixture was filtered and the filtrate was concentrated under vacuum to afford the title compound (65 mg, HCl salt) as white solid; ¹H NMR (400 MHz, CHLOROFORM-d) δ=3.54 (br s, 1H), 3.34 (br s, 1H), 2.93 (br s, 1H), 2.59 (br s, 1H), 2.25 (br s, 1H), 2.18-1.96 (m, 1H), 1.78 (br d, J=2.8 Hz, 1H), 0.96 (br s, 1H), 0.87 (br d, J=6.4 Hz, 1H).

Step C and D were according to Example 233 (step B and C) except that HCl was used instead of TFA in the last step. The title compound was obtained as light-yellow solid. ¹H NMR (400 MHz, DMSO-d₆) δ=9.71-9.65 (m, 1H), 7.59 (dd, J=6.0, 9.0 Hz, 1H), 7.24 (t, J=9.6 Hz, 1H), 6.99 (s, 2H), 6.63-6.55 (m, 1H), 5.34-5.11 (m, 1H), 5.07-4.94 (m, 1H), 4.82-4.68 (m, 1H), 4.60-4.44 (m, 2H), 4.43-4.26 (m, 1H), 4.22-4.07 (m, 1H), 3.96-3.72 (m, 5H), 3.60 (br d, J=17.2 Hz, 1H), 3.50-3.38 (m, 1H), 3.30-3.00 (m, 7H), 2.99-2.92 (m, 1H), 2.84-2.74 (m, 1H), 2.70-2.59 (m, 1H), 2.28-2.12 (m, 1H), 2.11-1.84 (m, 6H), 1.84-1.51 (m, 4H), 1.13-1.00 (m, 3H), 0.79-0.65 (m, 1H), 0.58 (br d, J=5.2 Hz, 1H); LCMS (ESI, M+1): m/z=725.4.

### Example 614

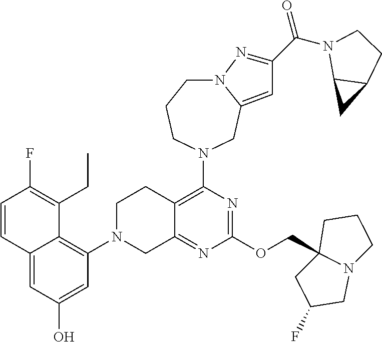 [SMILES: CCc1c(F)ccc2cc(O)cc(N3CCc4c(nc(OC[C@@]56CCCN5C[C@H](F)C6)nc4N4CCCn5nc(C(=O)N6CC[C@H]7C[C@H]76)cc5C4)C3)c12]

### ((1R,5S)-2-azabicyclo[3.1.0]hexan-2-yl)(5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

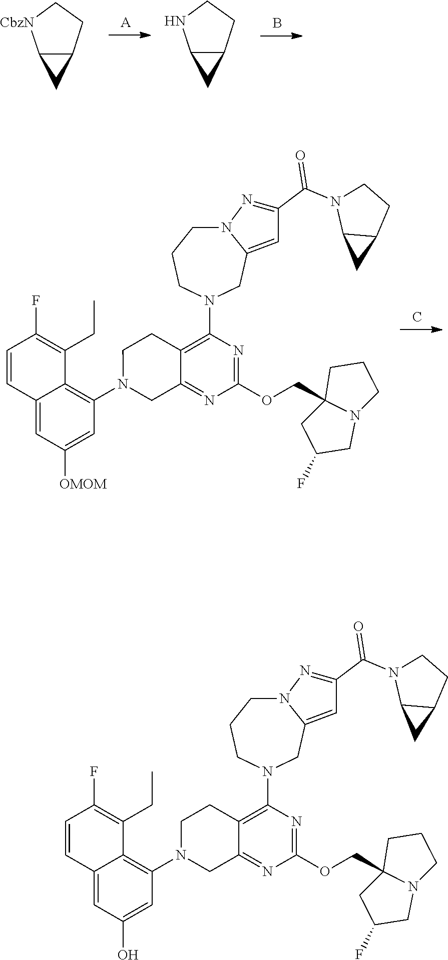

Step A. (1R,5S)-2-azabicyclo[3.1.0]hexane: To a solution of benzyl (1R,5S)-2-azabicyclo[3.1.0]hexane-2-carboxylate (100 mg, 1.0 equiv) in EtOH (10.0 mL) and HCl•dioxane (0.50 mL) was added Pd/C (10 mg, 10% purity) under N₂ atmosphere. The suspension was degassed and purged with H₂ for 3 times. The reaction was stirred under H₂ (15 Psi) at 20° C. for 2 hours. The mixture was filtered and the filtrate was concentrated under vacuum to afford the title compound (65.0 mg, crude, HCl salt) as white solid; ¹H NMR (400 MHz, CHLOROFORM-d) 6=3.54 (br s, 1H), 3.34 (br s, 1H), 2.93 (br s, 1H), 2.59 (br s, 1H), 2.25 (br s, 1H), 2.18-1.96 (m, 1H), 1.78 (br d, J=2.8 Hz, 1H), 0.96 (br s, 1H), 0.87 (br d, J=6.4 Hz, 1H).

Step B and C were according to Example 233 (step B and C) except that HCl was used instead of TFA in the last step. The title compound was obtained as yellow solid. ¹H NMR (400 MHz, DMSO-d₆) δ=9.71-9.65 (m, 1H), 7.59 (dd, J=6.0, 9.0 Hz, 1H), 7.24 (t, J=9.6 Hz, 1H), 6.99 (s, 2H), 6.63-6.55 (m, 1H), 5.34-5.11 (m, 1H), 5.07-4.94 (m, 1H), 4.82-4.68 (m, 1H), 4.60-4.44 (m, 2H), 4.43-4.26 (m, 1H), 4.22-4.07 (m, 1H), 3.96-3.72 (m, 5H), 3.60 (br d, J=17.6 Hz, 1H), 3.50-3.38 (m, 1H), 3.30-3.00 (m, 7H), 2.99-2.92 (m, 1H), 2.84-2.74 (m, 1H), 2.70-2.59 (m, 1H), 2.28-2.12 (m, 1H), 2.11-1.84 (m, 6H), 1.84-1.51 (m, 4H), 1.13-1.00 (m, 3H), 0.79-0.65 (m, 1H), 0.58 (br d, J=5.2 Hz, 1H); LCMS (ESI, M+1): m/z=725.4.

### Example 615

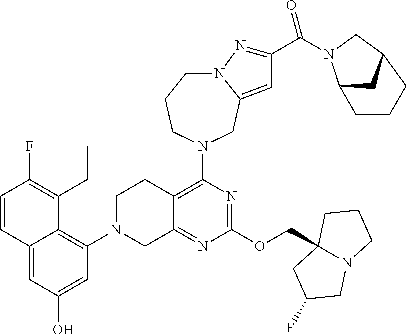 [SMILES: CCc1c(F)ccc2cc(O)cc(N3CCc4c(nc(OC[C@@]56CCCN5C[C@H](F)C6)nc4N4CCCn5nc(C(=O)N6C[C@@H]7CCC[C@H]6C7)cc5C4)C3)c12]

### ((1R,5S)-6-azabicyclo[3.2.1]octan-6-yl)(5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

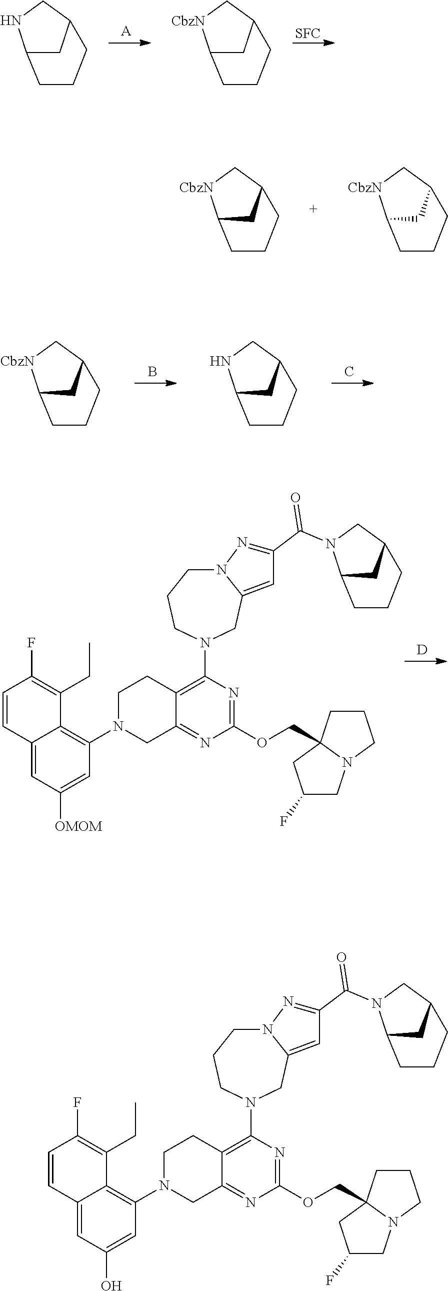

Step A. benzyl 6-azabicyclo[3.2]octane-6-carboxylate: To a solution of 6-azabicyclo[3.2.1]octane (2.00 g, 1.0 equiv) in DCM (20.0 mL) was added DIEA (6.97 g, 3.0 eq). The reaction was stirred at 0° C. for 10 minutes. Then CbzCl (3.68 g, 1.2 equiv) was added. The reaction was stirred at 0° C. for 1 hour. The mixture was extracted with EA (2×50 mL). The combined organic layers was washed with brine (20 mL), dried over anhydrous sodium sulfate, concentrated and purified with column chromatography (SiO₂, petroleum ether/ethyl acetate 100/1 to 30/1) followed with SFC separation [column: REGIS(S,S)WHELK-01, 250 mm×25 mm,10 μm; A: CO₂, B:IPA (0.1% NH₃H₂O); B %: 25% over 3.3 min] to afford two isomers.

benzyl (1R,5S)-6-azabicyclo[3.2.1]octane-6-carboxylate (0.84 g, 44% yield) as yellow oil; SFC>99% ee, column: (S,S)Whelk-01 100×4.6 mm I.D., 3.5 μm; mobile phase: 5% to 40% IPA (0.05% DEA) in CO₂, flow rate: 3 mL/min, detector: 220 nm, t_{R}: 2.789 min; H NMR (400 MHz, CHLOROFORM-d) δ=7.41-7.28 (m, 5H), 5.21-5.08 (m, 2H), 4.22-4.08 (m, 1H), 3.46 (m, 1H), 3.39-3.30 (m, 1H), 2.39 (m, 1H), 2.01-1.78 (m, 2H), 1.66-1.48 (m, 5H), 1.44-1.30 (m, 1H); LCMS (ESI, M+1): m/z=246.2.

benzyl (1S,5R)-6-azabicyclo[3.2.1]octane-6-carboxylate (0.84 g, 44% yield) as yellow oil; SFC>99% ee, column: (S,S)Whelk-01 100×4.6 mm I.D., 3.5 μm; mobile phase: 5% to 40% IPA (0.05% DEA) in CO₂, flow rate: 3 mL/min, detector: 220 nm, t_{R}: 2.965 min; ¹H NMR (400 MHz, CHLOROFORM-d) δ=7.42-7.28 (m, 5H), 5.25-5.07 (m, 2H), 4.16-4.09 (m, 1H), 3.51-3.40 (m, 1H), 3.39-3.30 (m, 1H), 2.39 (br s, 1H), 2.02-1.80 (m, 2H), 1.67-1.47 (m, 5H), 1.44-1.31 (m, 1H); LCMS (ESI, M+1): m/z=246.2.

Step B. (1R,5S)-6-azabicyclo[3.2.1]octane: To a solution of benzyl (1R,5S)-6-azabicyclo[3.2.1]octane-6-carboxylate (1.90 g, 1.0 equiv) in MeOH (20.0 mL) was added Pd/C (200 mg, 10% purity) and HCl•dioxane (4 M, 4.00 mL) under N₂ atmosphere. The suspension was degassed and purged with H₂ for 3 times. The reaction was stirred under H₂ (15 Psi) at 25° C. for 5 hours. The mixture was filtered and the filtrate was concentrated under vacuum to afford the title compound (1.14 g, HCl salt) as white solid; ¹H NMR (400 MHz, METHANOL-d₄) δ=3.96 (t, J=4.8 Hz, 1H), 3.34-3.34 (m, 1H), 3.30 (br d, J=2.0 Hz, 1H), 2.61 (br s, 1H), 2.00-1.83 (m, 3H), 1.78-1.61 (m, 5H).

Step C and D were according to Example 233 (step B and C) except that HCl was used instead of TFA in the last step. The title compound was obtained as white solid. ¹H NMR (400 MHz, DMSO-d₆) δ=9.69 (s, 1H), 7.59 (dd, J=6.0, 8.9 Hz, 1H), 7.24 (t, J=9.4 Hz, 1H), 6.99 (s, 2H), 6.63-6.53 (m, 1H), 5.35-5.11 (m, 1H), 5.10-4.92 (m, 1H), 4.89-4.78 (m, 1H), 4.76-4.69 (m, 1H), 4.62-4.30 (m, 2H), 4.28-4.07 (m, 1H), 3.97-3.69 (m, 5H), 3.59 (br d, J=16.8 Hz, 1H), 3.52-3.35 (m, 2H), 3.31-3.14 (m, 3H), 3.13-2.90 (m, 4H), 2.83-2.73 (m, 1H), 2.71-2.58 (m, 1H), 2.44-2.31 (m, 1H), 2.27-2.12 (m, 1H), 2.10-2.01 (m, 1H), 2.00-1.86 (m, 4H), 1.86-1.65 (m, 4H), 1.64-1.29 (m, 6H), 1.13-0.99 (m, 3H); LCMS (ESI, M+1): m/z=753.4.

### Example 616

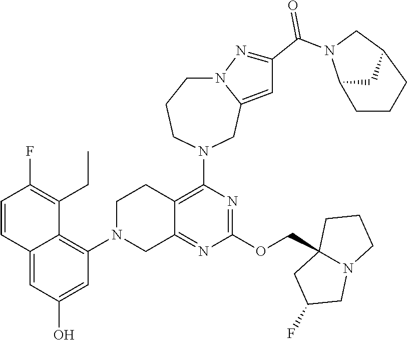 [SMILES: CCc1c(F)ccc2cc(O)cc(N3CCc4c(nc(OC[C@@]56CCCN5C[C@H](F)C6)nc4N4CCCn5nc(C(=O)N6C[C@H]7CCC[C@@H]6C7)cc5C4)C3)c12]

### ((1S,5R)-6-azabicyclo[3.2.1]octan-6-yl)(5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

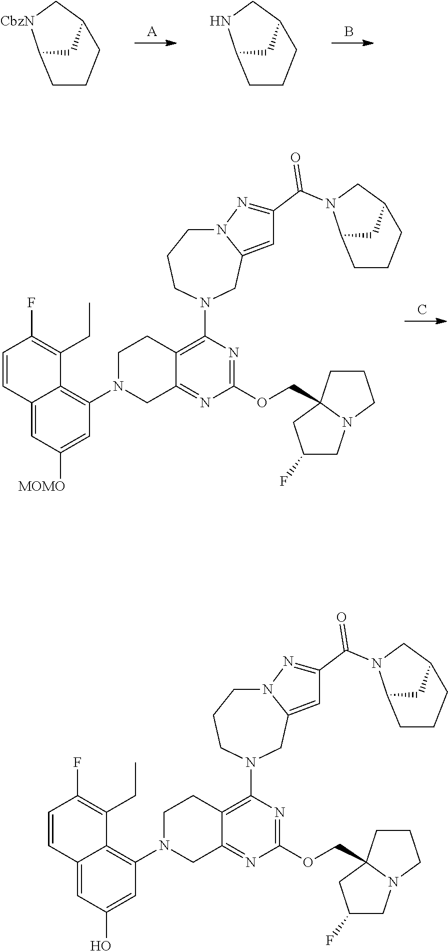

Step A. (1S,5R)-6-azabicyclo[3.2.1]octane: To a solution of benzyl (1S,5R)-6-azabicyclo[3.2.1]octane-6-carboxylate (2.10 g, 1.0 equiv) in MeOH (20.0 mL) was added Pd/C (210 mg, 10% purity) and HCl•dioxane (4 M, 4.00 mL) under N₂ atmosphere. The suspension was degassed and purged with H₂ for 3 times. The reaction was stirred under H₂ (15 Psi) at 25° C. for 5 hours. The mixture was filtered and the filtrate was concentrated under vacuum to afford the title compound (1.20 g, crude, HCl salt) as white solid. ¹H NMR (400 MHz, METHANOL-d₄) δ=3.93 (t, J=4.8 Hz, 1H), 3.31 (s, 1H), 3.29-3.28 (m, 1H), 2.57 (br s, 1H), 1.96-1.78 (m, 3H), 1.74-1.58 (m, 5H).

Step B and C were according to Example 233 (step B and C) except that HCl was used instead of TFA in the last step. The title compound was obtained as white solid. ¹H NMR (400 MHz, DMSO-d₆) δ=9.76-9.65 (m, 1H), 7.59 (dd, J=6.0, 9.0 Hz, 1H), 7.24 (t, J=9.4 Hz, 1H), 6.99 (s, 2H), 6.64-6.53 (m, 1H), 5.35-5.13 (m, 1H), 5.08-4.93 (m, 1H), 4.90-4.77 (m, 1H), 4.77-4.69 (m, 1H), 4.59-4.32 (m, 2H), 4.26-4.06 (m, 1H), 3.95-3.70 (m, 5H), 3.59 (br d, J=17.2 Hz, 1H), 3.51-3.35 (m, 2H), 3.31-3.14 (m, 3H), 3.13-2.91 (m, 4H), 2.86-2.73 (m, 1H), 2.71-2.60 (m, 1H), 2.44-2.31 (m, 1H), 2.26-2.12 (m, 1H), 2.09-1.86 (m, 5H), 1.86-1.64 (m, 4H), 1.63-1.29 (m, 6H), 1.13-0.98 (m, 3H); LCMS (ESI, M+1): m/z=753.4.

### Example 617

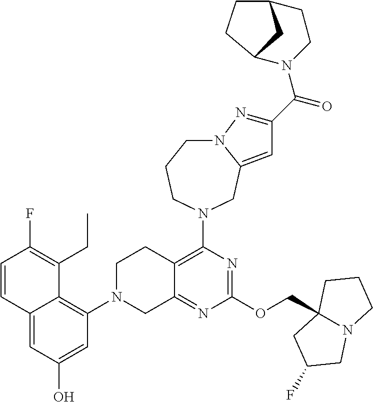 [SMILES: CCc1c(F)ccc2cc(O)cc(N3CCc4c(nc(OC[C@@]56CCCN5C[C@H](F)C6)nc4N4CCCn5nc(C(=O)N6CC[C@H]7CC[C@@H]6C7)cc5C4)C3)c12]

### ((1R,5R)-2-azabicyclo[3.2.1]octan-2-yl)(5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

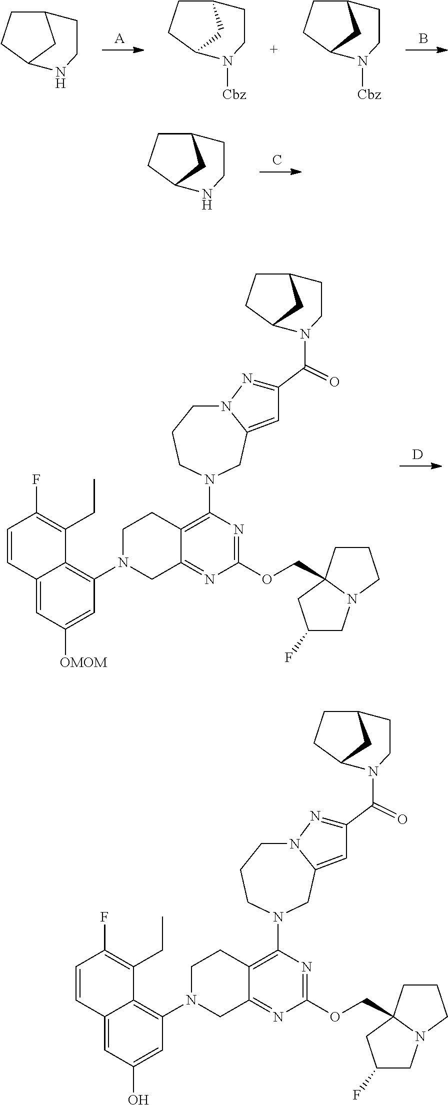

Step A. (1R,5R)-benzyl 2-azabicyclo[3.2.1]octane-2-carboxylate and (1S,5S)-benzyl 2-azabicyclo[3.2.1]octane-2-carboxylate: To a solution of 2-azabicyclo[3.2.1]octane hydrochloride (500 mg, 1.0 equiv, HCl) and TEA (1.71 g, 5.0 equiv) in dichloromethane (5 mL) was added CbzCl (1.16 g, 2.0 equiv) at 0° C. The reaction was stirred at 0° C. for 8 hours. The mixture was diluted with water (5 mL) and extracted with dichloromethane (3×5 mL). The combined organic layers were dried over anhydrous sodium sulfate, concentrated and purified with column chromatography [SiO₂, Petroleum ether/Ethyl acetate=100/1 to 10/1] and SFC [DAICEL CHIRALPAK AD-H (250 mm×30 mm, 5 μm); A: [0.1% NH₃H₂O MEOH]; B %: 15%-15% over 2 min] to afford two peaks. Peak 1 (1R,5R)-benzyl 2-azabicyclo[3.2.1]octane-2-carboxylate (250 mg, 41% yield) as yellow liquid; LCMS (ESI, M+1): m/z=246.1; SFC: 100% de, column: ChiralpakAD-3 50×4.6 mm I.D., 3 μm Mobile phase: Phase A for CO₂, and Phase B for MeOH (0.05% DEA); Gradient elution: MeOH (0.05% DEA) in CO₂ from 5% to 40% Flow rate: 3 mL/min; Detector: PDA Column Temp: 35° C.; Back Pressure: 100Bar, t_{R}: 0.893 min. Peak 2 (1S,5S)-benzyl 2-azabicyclo[3.2.1]octane-2-carboxylate (250 mg, 40% yield) as yellow liquid; LCMS (ESI, M+1): m/z=246.1; SFC: 98.9% de, column: ChiralpakAD-3 50×4.6 mm I.D., 3 μm Mobile phase: Phase A for CO₂, and Phase B for MeOH (0.05% DEA); Gradient elution: MeOH (0.05% DEA) in CO₂ from 5% to 40% Flow rate: 3 mL/min; Detector: PDA Column Temp: 35° C.; Back Pressure: 100Bar, t_{R}: 0.946 min.

Step B. (1R,5R)-2-azabicyclo[3.2.I]octane hydrochloride: To a solution of (1R,5R)-benzyl 2-azabicyclo[3.2.1]octane-2-carboxylate (250 mg, 1.0 equiv) and HCl•MeOH (4 M, 509 μL, 2.0 equiv) in MeOH (5 mL) was added Pd/C (50.0 mg, 10% purity, 1.0 equiv). The reaction was degassed and purged with H₂ 3 times. The reaction was stirred under H₂ (15 psi) at 20° C. for 12 hours. The mixture was filtered and the filter was concentrated to afford the title compound (150 mg, crude, HCl) as yellow solid.

Step C and D were according to Example 233 (step B and C) except that HCl was used instead of TFA in the last step. The title compound was obtained as yellow solid; ¹H NMR (400 MHz, METHANOL-d₄) δ=7.51 (dd, J=6.0, 8.8 Hz, 1H), 7.14 (t, J=9.6 Hz, 1H), 6.97 (s, 2H), 6.60-6.49 (m, 1H), 5.47-5.25 (m, 1H), 5.08-4.92 (m, 3H), 4.57-4.48 (m, 2H), 4.37-4.12 (m, 4H), 4.11-3.93 (m, 2H), 3.67 (br d, J=17.6 Hz, 1H), 3.57-3.35 (m, 6H), 3.28-2.95 (m, 4H), 2.80-2.63 (m, 1H), 2.48-2.14 (m, 5H), 2.13-1.65 (m, 9H), 1.64-1.34 (m, 3H), 1.10 (q, J=6.8 Hz, 3H); LCMS (ESI, M+1): m/z=753.6.

### Example 618

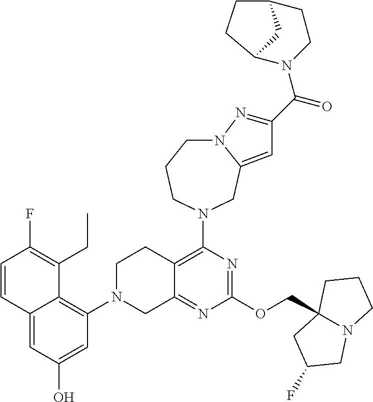 [SMILES: CCc1c(F)ccc2cc(O)cc(N3CCc4c(nc(OC[C@@]56CCCN5C[C@H](F)C6)nc4N4CCCn5nc(C(=O)N6CC[C@@H]7CC[C@H]6C7)cc5C4)C3)c12]

### ((1S,5S)-2-azabicyclo[3.2.1]octan-2-yl)(5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

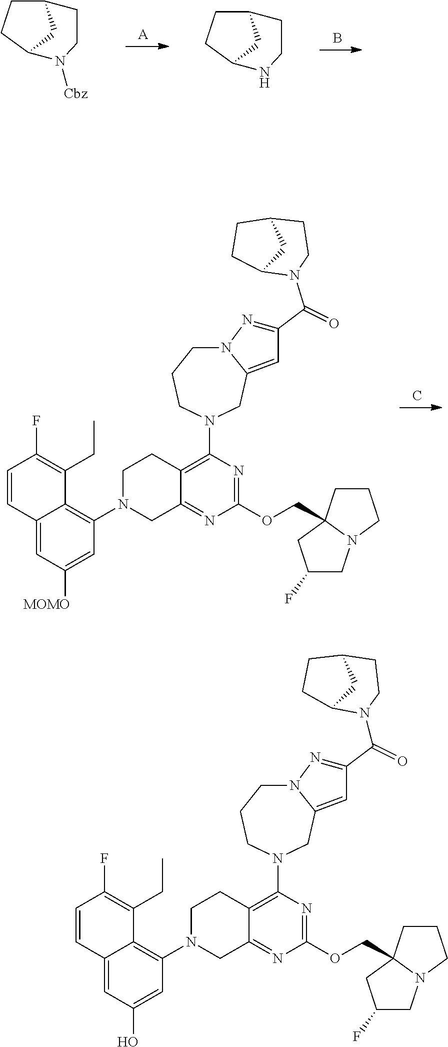

Step A. (1S,5S)-2-azabicyclo[3.2.1]octane hydrochloride: To a solution of (1S,5S)-benzyl 2-azabicyclo[3.2.1]octane-2-carboxylate (250 mg, 1.0 equiv) and HCl-MeOH (4 M, 509 μL, 2.0 equiv) in MeOH (5 mL) was added Pd/C (50.0 mg, 10% purity, 1.0 equiv). The reaction was degassed and purged with H₂3 times. The reaction was stirred under H₂ (15 psi) at 20° C. for 12 hours. The mixture was filtered and the filter was concentrated to afford the title compound (150 mg, crude, HCl) as yellow solid.

Step B and C were according to Example 233 (step B and C) except that HCl was used instead of TFA in the last step. The title compound was obtained as pink solid (FA salt). ¹H NMR (400 MHz, METHANOL-d₄) δ=7.51 (dd, J=6.0, 9.2 Hz, 1H), 7.14 (t, J=9.2 Hz, 1H), 7.05-6.81 (m, 2H), 6.62-6.49 (m, 1H), 5.52-5.28 (m, 1H), 5.11-4.91 (m, 3H), 4.59-4.46 (m, 2H), 4.37-4.12 (m, 4H), 4.05 (br dd, J=9.6, 17.6 Hz, 2H), 3.73-3.43 (m, 5H), 3.42-3.34 (m, 2H), 3.29-2.95 (m, 4H), 2.81-2.66 (m, 1H), 2.52-2.16 (m, 5H), 2.16-1.63 (m, 9H), 1.62-1.34 (m, 3H), 1.09 (td, J=7.2, 11.2 Hz, 3H); LCMS (ESI, M+1): m/z=753.4.

### Example 619

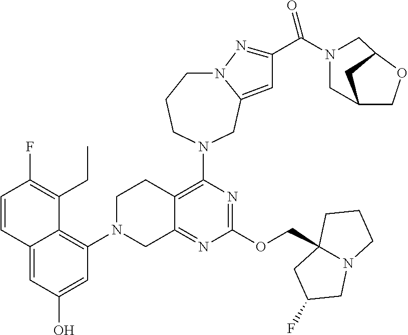 [SMILES: CCc1c(F)ccc2cc(O)cc(N3CCc4c(nc(OC[C@@]56CCCN5C[C@H](F)C6)nc4N4CCCn5nc(C(=O)N6C[C@H]7CO[C@@H](C7)C6)cc5C4)C3)c12]

### ((1S,5R)-6-oxa-3-azabicyclo[3.2.1]octan-3-yl)(5-(7-(8-ethyl-7-fluoro-³-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

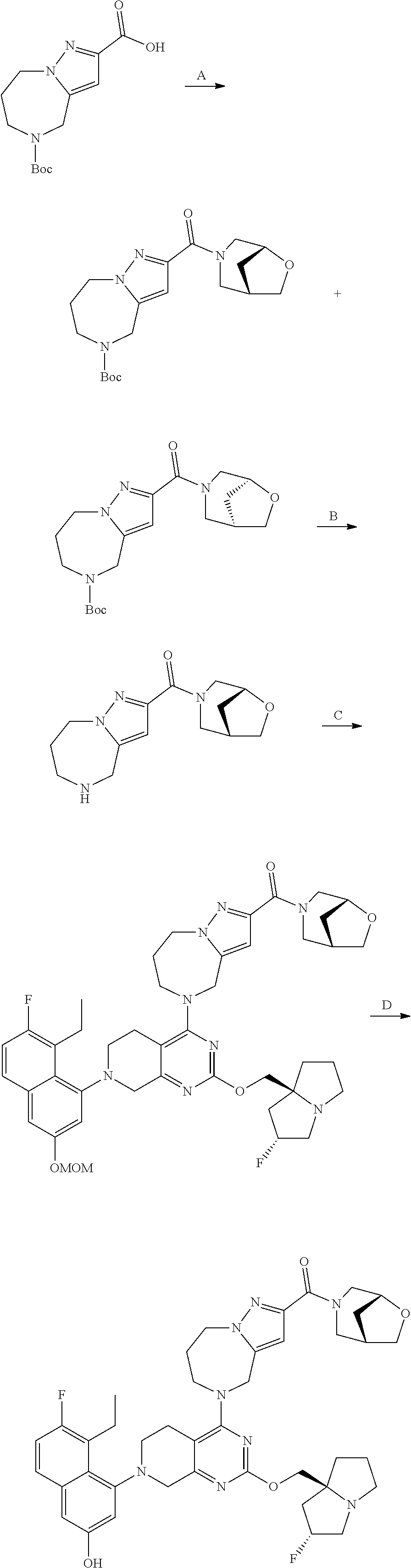

Step A. tert-butyl 2-((1S,5R)-6-oxa-3-azabicyclor3.2.11octane-3-carbonyl)-7,8-dihydro-4H-pyrazolo[1,5-a][1,4]diazepine-5(6H)-carboxylate: To a solution of 5-(tert-butoxycarbonyl)-5,6,7,8-tetrahydro-4H-pyrazolo[1,5-a][1,4]diazepine-2-carboxylic acid (3.00 g, 1.0 equiv) and 6-oxa-3-azabicyclo[3.2.1]octane (1.91 g, 1.2 equiv, HCl) in DMF (50 mL) were added DIEA (6.89 g, 5.0 equiv) and HATU (8.11 g, 2.0 equiv) at 0° C. The reaction was stirred at 25° C. for 1 hour. The mixture was diluted with water (100 mL) and extracted with ethyl acetate (200 mL). The organic layer was dried over sodium sulfate, concentrated and purified with reversed-phase HPLC [Cl8, 0.1% formic acid condition] and followed by SFC [column: DAICEL CHIRALPAK AD, 250×30 mm, 10 μm; A: CO₂, B: MeOH (0.1% NH₃H₂O), B %: 50% B over 4.0 min] to afford two isomers. tert-butyl 2-((1S,5R)-6-oxa-3-azabicyclo[3.2.1]octane-3-carbonyl)-7,8-dihydro-4H-pyrazolo[1,5-a][1,4]diazepine-5(6H)-carboxylate (1.67 g, 42% yield) as yellow solid; SFC: >99% ee, column: Chiralpak AD-3 50×4.6 mm ID., 3 Rm, mobile phase 5%-40% MeOH(0.05% DEA) in CO₂, flow rate: 3 mL/min, detector: 220 nm, t_{R}: 1.363 min; LCMS (ESI, M+1): m/z=377.4. tert-butyl 2-((1R,5S)-6-oxa-3-azabicyclo[3.2.1]octane-3-carbonyl)-7,8-dihydro-4H-pyrazolor1,5-al[1,4]diazepine-5(6H)-carboxylate (1.60 g, 40% yield) as yellow solid; SFC: >99% ee, column: Chiralpak AD-3 50×4.6 mm I.D., 3 μm, mobile phase 5%-40% MeOH(0.05% DEA) in CO₂, flow rate: 3 mL/min, detector: 220 nm, t_{R}: 1.625 min; LCMS (ESI, M+1): m/z=377.4.

Step B. ((1S,5R)-6-oxa-3-azabicyclo[3.2.1]octan-3-yl)(5,6,7,8-tetrahydro-4H-pyrazolo[1,5-a][1,4]diazepin-2-yl)methanone: To a solution of tert-butyl 2-((1S,5R)-6-oxa-3-azabicyclo[3.2.1]octane-3-carbonyl)-7,8-dihydro-4H-pyrazolo[1,5-a][1,4]diazepine-5(6H)-carboxylate (1.67 g, 1.0 equiv) in MeOH (10 mL) was added HCl•MeOH (4 M, 20 mL, 18 equiv) at 0° C. The reaction was stirred at 25° C. for 3 hours. The mixture was concentrated under vacuum to afford the title compound (1.39 g, crude, HCl) as yellow solid.

Step C and D were according to Example 233 (step B and C) except that HCl was used instead of TFA in the last step. The title compound was obtained as yellow solid; ¹H NMR (400 MHz, methanol-d₄) δ=7.50 (dd, J=5.6, 8.8 Hz, 1H), 7.13 (t, J=9.6 Hz, 1H), 6.96 (s, 2H), 6.62-6.54 (m, 1H), 5.35-5.16 (m, 1H), 5.10-4.92 (m, 1H), 4.82-4.71 (m, 1H), 4.65-4.44 (m, 3H), 4.42-4.12 (m, 3H), 4.11-3.83 (m, 5H), 3.82-3.73 (m, 1H), 3.66 (br d, J=17.6 Hz, 1H), 3.51 (br d, J=9.6 Hz, 1H), 3.46-3.34 (m, 2H), 3.29-3.09 (m, 6H), 3.08-2.84 (m, 2H), 2.70 (br d, J=13.6 Hz, 1H), 2.62-2.45 (m, 1H), 2.37-2.10 (m, 3H), 2.10-2.00 (m, 2H), 1.99-1.77 (m, 5H), 1.17-1.03 (m, 3H); SFC: >99% ee, column: Chiralpak IC-3 50×4.6 mm I.D., 3 μm, mobile phase 100% ACN (0.05% DEA) in CO₂, flow rate: 1.0 mL/min, detector: 220 nm, t_{R}: 6.315 min; LCMS (ESI, M+1): m/z=755.6.

### Example 620

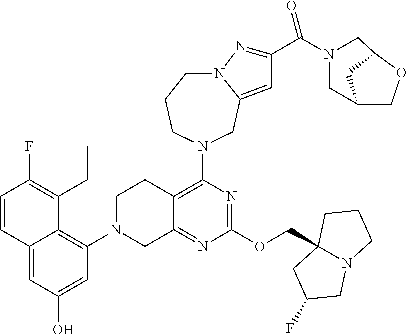 [SMILES: CCc1c(F)ccc2cc(O)cc(N3CCc4c(nc(OC[C@@]56CCCN5C[C@H](F)C6)nc4N4CCCn5nc(C(=O)N6C[C@@H]7CO[C@H](C7)C6)cc5C4)C3)c12]

### ((1R,5S)-6-oxa-3-azabicyclo[3.2.1]octan-3-yl)(5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

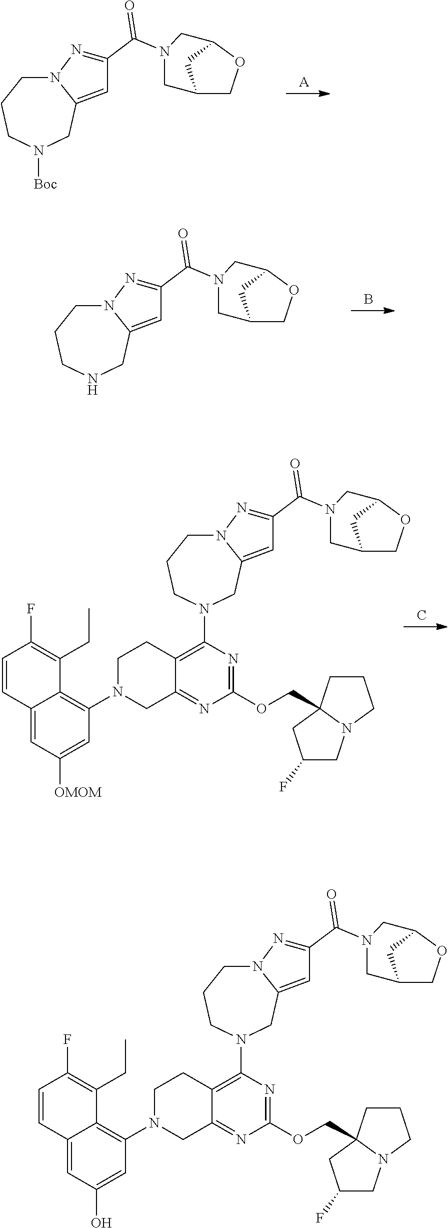

Step A. ((1R,5S)-6-oxa-3-azabicyclo[3.2.1]octan-3-yl)(5,6,7,8-tetrahydro-4H-pyrazolo[1,5-a][1,4]diazepin-2-yl)methanone: To a solution of tert-butyl 2-((1R,5S)-6-oxa-3-azabicyclo[3.2.1]octane-3-carbonyl)-7,8-dihydro-4H-pyrazolo[1,5-a][1,4]diazepine-5(6H)-carboxylate (1.60 g, 1.0 equiv) in MeOH (10 mL) was added HCl•MeOH (4 M, 20 mL, 19 equiv) at 0° C. The reaction was stirred at 25° C. for 3 hours. The mixture was concentrated under vacuum to afford the title compound (1.33 g, crude, HCl) as yellow solid.

Step B and C were according to Example 233 (step B and C) except that HCl was used instead of TFA in the last step. The title compound was obtained as yellow solid; ¹H NMR (400 MHz, methanol-d₄) δ=7.50 (dd, J=5.6, 8.8 Hz, 1H), 7.14 (t, J=9.2 Hz, 1H), 6.96 (s, 2H), 6.62-6.53 (m, 1H), 5.36-5.15 (m, 1H), 5.10-4.94 (m, 1H), 4.83-4.74 (m, 1H), 4.64-4.46 (m, 3H), 4.42-4.13 (m, 3H), 4.12-3.83 (m, 5H), 3.82-3.75 (m, 1H), 3.66 (dd, J=3.2, 17.6 Hz, 1H), 3.57-3.47 (m, 1H), 3.46-3.34 (m, 2H), 3.30-3.11 (m, 6H), 3.09-2.84 (m, 2H), 2.71 (br d, J=14.4 Hz, 1H), 2.62-2.47 (m, 1H), 2.36-2.11 (m, 3H), 2.11-2.02 (m, 2H), 2.00-1.78 (m, 5H), 1.18-1.02 (m, 3H); SFC: >99% ee, column: Chiralpak IC-3 50×4.6 mm I.D., 3 μm, mobile phase:100% ACN (0.05% DEA) in CO₂, flow rate:1.0 mL/min, detector: 220 nm, t_{R1}: 2.418 min, t_{R}2: 2.673 min; LCMS (ESI, M+1, M/2+1): m/z=755.4. 378.3.

### Example 621

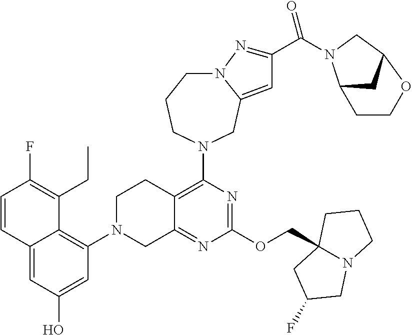 [SMILES: CCc1c(F)ccc2cc(O)cc(N3CCc4c(nc(OC[C@@]56CCCN5C[C@H](F)C6)nc4N4CCCn5nc(C(=O)N6C[C@H]7C[C@@H]6CCO7)cc5C4)C3)c12]

### ((1R,5S)-2-oxa-6-azabicyclo[3.2.1]octan-6-yl)(5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

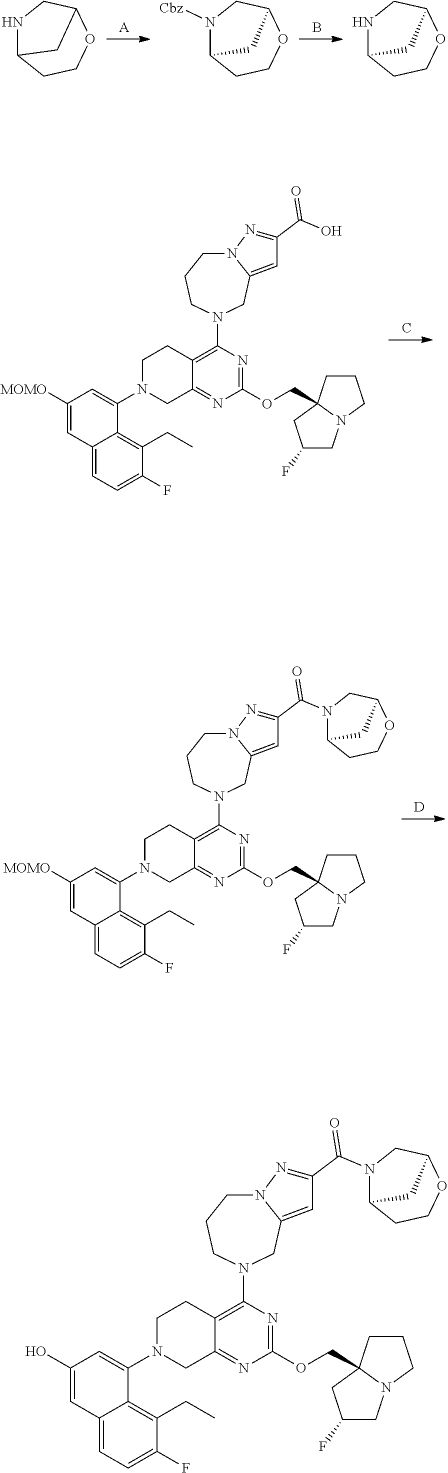

Step A. benzyl (1S,5R)-2-oxa-6-azabicyclo[3.2.1]octane-6-carboxylate: To a solution of 2-oxa-6-azabicyclo[3.2.1]octane (500 mg, 1.0 equiv, HCl) and TEA (1.35 g, 4.0 equiv) in DCM (10 mL) was added benzyl carbonochloridate (855 mg, 1.5 equiv) at −40° C. The reaction was stirred at −40° C. for 0.5 hours and 0° C. for 6 hours. The mixture was diluted with water (10 mL) and extracted with DCM (4×10 mL). The combined organic layers were washed with brine (20 mL), dried over anhydrous sodium sulfate, concentrated and purified with column chromatography [SiO₂, Petroleum ether/Ethyl acetate=5/1 to 1/1] and SFC [column: DAICEL CHIRALPAK IC (250 mm×30 mm, 10 μm); A: (0.1% NH₃H₂O IPA); B %: 35%-35% over 3.0 min] to afford two peaks.

Peak 1: benzyl (1S,5R)-2-oxa-6-azabicyclo[3.2.1]octane-6-carboxylate (200 mg, 33% yield) as yellow liquid; LCMS (ESI, M+1): m/z=248.1; SFC [Column: Cellulose-2 50×4.6 mm ID, 3 μm: Mobile phase: Phase A for CO₂ and Phase B for MeOH (0.05% DEA); Gradient elution: MeOH (0.05% DEA) in CO₂ from 5% to 40% Flow rate: 3 mL/min; Detector: PDA Column Temp: 35C; Back Pressure: 100Bar]. t_{R}: 1.051 min.

Peak 2: benzyl (1R,5S)-2-oxa-6-azabicyclo[3.2.1]octane-6-carboxylate (154 mg, 24% yield) as yellow liquid; LCMS (ESI, M+1): m/z=248.1; SFC [Column: Cellulose-2 50×4.6 mm ID, 3 μm, Mobile phase: Phase A for CO₂ and Phase B for MeOH (0.05% DEA); Gradient elution: MeOH (0.05% DEA) in CO₂ from 5% to 40% Flow rate: 3 mL/min; Detector: PDA Column Temp: 35C; Back Pressure: 100Bar]. t_{R}: 1.255 min.

Step B. (1S,5R)-2-oxa-6-azabicyclo[3.2.1]octane: To a solution of benzyl (lS,5R)-2-oxa-6-azabicyclo[3.2.1]octane-6-carboxylate (200 mg, 1.0 equiv) in MeOH (2 mL) were added Pd/C (50.0 mg, 10% purity) and HCl•MeOH (4 M, 1.0 mL, 5.0 equiv) under N₂ atmosphere. The reaction was degassed and purged with H₂ 3 times. The reaction was stirred under H₂ (15 Psi or atm) at 25° C. for 12 hours. The mixture was filtered and concentrated to afford the title compound (100 mg, crude, HCl) as yellow oil.

Step C and D were according to Example 233 (step B and C) except that HCl was used instead of TFA in the last step. The title compound was obtained as light yellow solid (FA salt). ¹H NMR (400 MHz, METHANOL-d₄) δ=7.53-7.49 (m, 1H), 7.17-7.12 (m, 1H), 6.98-6.97 (m, 2H), 6.77-6.74 (m, 1H), 5.51-5.30 (m, 1H), 5.29-4.95 (m, 2H), 4.66-4.44 (m, 4H), 4.32-4.23 (m, 2H), 4.22-4.14 (m, 1H), 4.03-4.02 (m, 2H), 3.90-3.43 (m, 9H), 3.39-3.36 (m, 2H), 3.28-3.12 (m, 3H), 2.74 (br d, J=14.4 Hz, 1H), 2.51-2.37 (m, 1H), 2.36-2.19 (m, 3H), 2.18-2.00 (m, 4H), 1.98-1.78 (m, 4H), 1.15-1.05 (m, 3H); LCMS (ESI, M+1): m/z=755.3.

### Example 622

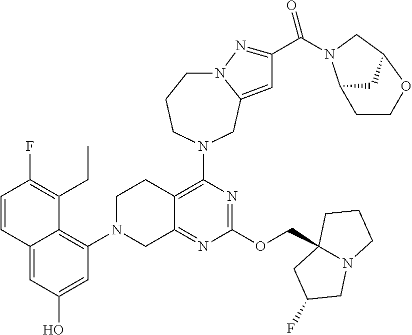 [SMILES: CCc1c(F)ccc2cc(O)cc(N3CCc4c(nc(OC[C@@]56CCCN5C[C@H](F)C6)nc4N4CCCn5nc(C(=O)N6C[C@@H]7C[C@H]6CCO7)cc5C4)C3)c12]

### ((1S,5R)-2-oxa-6-azabicyclo[3.2.1]octan-6-yl)(*5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

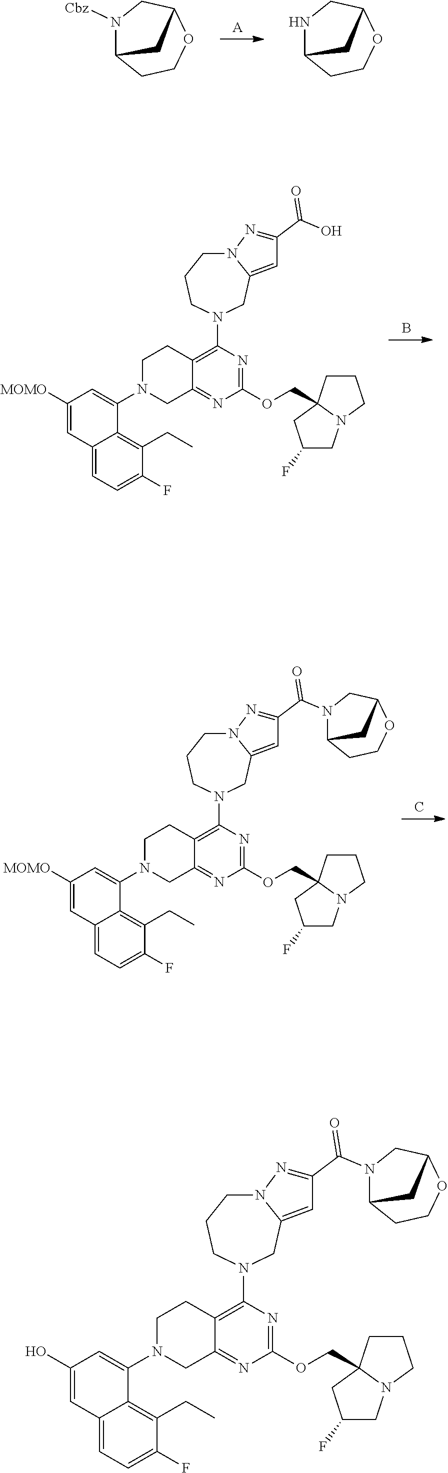

Step A. (1R,5S)-2-oxa-6-azabicyclo[3.2.1]octane: To a solution of benzyl (1R,5S)-2-oxa-6-azabicyclo[3.2.1]octane-6-carboxylate (150 mg, 1.0 equiv) in MeOH (2.0 mL) were added Pd/C (50.0 mg, 10% purity) and HCl•MeOH (4 M, 758 μL, 5.0 equiv) under N₂ atmosphere. The reaction was stirred under H₂ (15 Psi or atm) at 25° C. for 12 hours. The mixture was filtered and concentrated to afford the title compound (100 mg, crude, HCl) as yellow solid.

Step B and C were according to Example 233 (step B and C) except that HCl was used instead of TFA in the last step. The title compound was obtained as orange solid (FA salt). ¹H NMR (400 MHz, METHANOL-d₄) δ=7.53-7.49 (m, 1H), 7.16-7.12 (m, 1H), 6.98-6.97 (m, 2H), 6.79-6.74 (m, 1H), 5.61-5.39 (m, 1H), 5.34-4.97 (m, 2H), 4.62-4.42 (m, 4H), 4.42-4.25 (m, 2H), 4.24-4.15 (m, 1H), 4.11-3.99 (m, 2H), 3.89-3.60 (m, 9H), 3.32-3.29 (m, 3H), 3.28-3.12 (m, 2H), 2.75 (br d, J=14.4 Hz, 1H), 2.65-2.42 (m, 2H), 2.39-2.32 (m, 1H), 2.29-2.00 (m, 5H), 2.00-1.76 (m, 4H), 1.15-1.05 (m, 3H); LCMS (ESI, M+1): m/z=755.3.

### Example 623

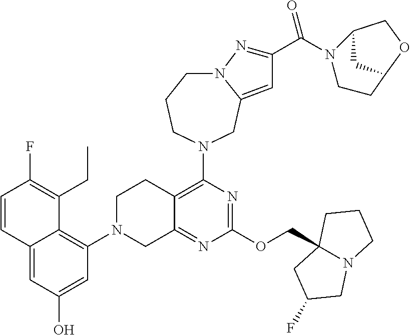 [SMILES: CCc1c(F)ccc2cc(O)cc(N3CCc4c(nc(OC[C@@]56CCCN5C[C@H](F)C6)nc4N4CCCn5nc(C(=O)N6CC[C@@H]7C[C@@H]6CO7)cc5C4)C3)c12]

### ((1R,5S)-6-oxa-2-azabicyclo[3.2.1]octan-2-yl)(5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

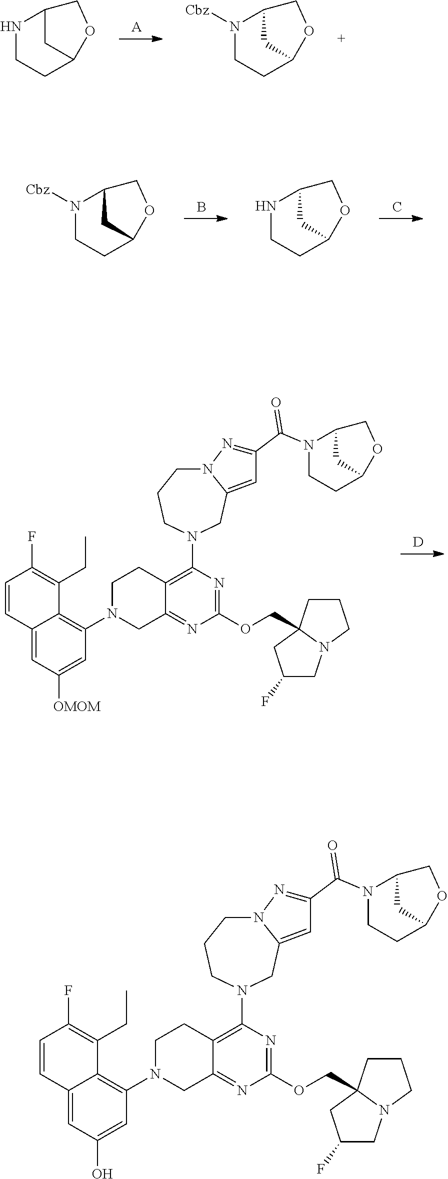

Step A. (1R,5S)-benzyl 6-oxa-2-azabicyclo[3.2.1]octane-2-carboxylate and (1S,5R)-benzyl 6-oxa-2-azabicyclo[3.2.1]octane-2-carboxylate: To a solution of 6-oxa-2-azabicyclo[3.2.1]octane hydrochloride (4.00 g, 1.0 equiv, HCl) in dichloromethane (40 mL) was added TEA (13.5 g, 5.0 equiv) and CbzCl (4.56 g, 1.0 equiv) at −40° C. The reaction was stirred at −40° C. for 0.5 hours. The mixture was diluted with water (20 mL) and extracted with dichloromethane (3×20 mL). The combined organic layers were dried over anhydrous sodium sulfate, concentrated and purified with column chromatography [SiO₂, Petroleum ether/Ethyl acetate=100/1 to 10/1] and SFC [DAICEL CHIRALCEL OX (250 mm×30 mm, 10 μm); A: 0.1% NH₃•H₂O, B: IPA; B %: 15%-15% over 2.9 min] to afford two peaks.

Peak 1 (1R,5S)-benzyl 6-oxa-2-azabicyclo[3.2.1]octane-2-carboxylate (2.00 g, 37% yield) as colorless liquid; LCMS (ESI, M+1): m/z=248.1; SFC: 99.3% de, column: Chiralcel OX-3 50×4.6 mm I.D., 3 μm Mobile phase: Phase A for CO₂, and Phase B for IPA (0.05% DEA); Gradient elution: IPA (0.05% DEA) in CO₂ from 5% to 40% Flow rate: 3 mL/min; Detector: PDA; Column Temp: 35° C.; Back Pressure: 100 Bar, t_{R}: 1.058 min.

Peak 2 (1S,5R)-benzyl 6-oxa-2-azabicyclo[3.2.1]octane-2-carboxylate (2.20 g, 41% yield) as colorless liquid; LCMS (ESI, M+1): m/z=248.1; SFC: 98.0% de, column: Chiralcel OX-3 50×4.6 mm I.D., 3 μm Mobile phase: Phase A for CO₂, and Phase B for IPA (0.05% DEA); Gradient elution: IPA (0.05% DEA) in CO₂ from 5% to 40% Flow rate: 3 mL/min; Detector: PDA; Column Temp: 35° C.; Back Pressure: 100 Bar, t_{R}: 1.143 min.

Step B. (1R,5S)-6-oxa-2-azabicyclo[3.2.1]octane hydrochloride: To a solution of (1R,5S)-benzyl 6-oxa-2-azabicyclo[3.2.1]octane-2-carboxylate (2.00 g, 1.0 equiv) and HCl•MeOH (4 M, 4.04 mL, 2.0 equiv) in MeOH (20 mL) was added Pd/C (500 mg, 10% purity, 1.0 equiv). The reaction was degassed and purged with H₂ 3 times. The reaction was stirred under H₂ (15 psi) at 20° C. for 12 hours. The mixture was filtered and the filter was concentrated to afford the title compound (1.00 g, crude, HCl) as yellow solid.

Step C and D were according to Example 233 (step B and C) except that HCl was used instead of TFA in the last step. The title compound was obtained as off-white solid; ¹H NMR (400 MHz, METHANOL-d₄) δ=7.51 (dd, J=6.0 9.2 Hz, 1H), 7.14 (t, J=9.2 Hz, 1H), 7.02-6.91 (m, 2H), 6.60 (dd, J=5.2, 11.6 Hz, 1H), 5.53-5.29 (m, 1H), 5.28-5.15 (m, 1H), 5.09-4.91 (m, 2H), 4.70-4.47 (m, 4H), 4.47-4.12 (m, 2H), 4.12-3.82 (m, 6H), 3.67 (br dd, J=5.6, 18.0 Hz, 1H), 3.64-3.41 (m, 2H), 3.40-3.35 (m, 1H), 3.26-3.12 (m, 5H), 2.98 (dt, J=5.6, 9.2 Hz, 1H), 2.72 (br d, J=14.8 Hz, 1H), 2.41-2.17 (m, 2H), 2.16-2.03 (m, 3H), 2.02-1.83 (m, 5H), 1.82-1.52 (m, 2H), 1.10 (td, J=7.2, 10.0 Hz, 3H); LCMS (ESI, M+1): m/z=755.5.

### Example 624

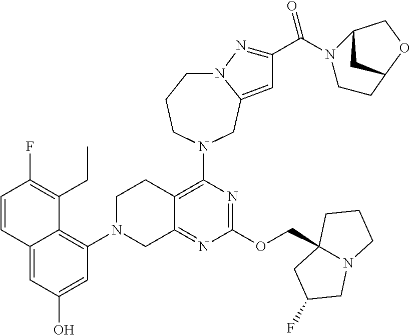 [SMILES: CCc1c(F)ccc2cc(O)cc(N3CCc4c(nc(OC[C@@]56CCCN5C[C@H](F)C6)nc4N4CCCn5nc(C(=O)N6CC[C@H]7C[C@H]6CO7)cc5C4)C3)c12]

### ((1 S,5R)-6-oxa-2-azabicyclo[3.2.1]octan-2-yl)(5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepin-2-yl)methanone

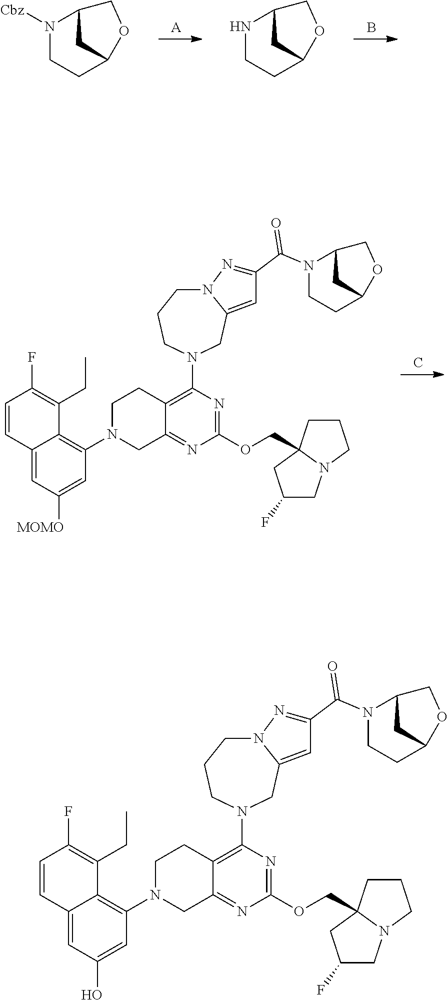

Step A. (1S,5R)-6-oxa-2-azabicyclo[3.2.1]octane hydrochloride: To a solution of (1S,5R)-benzyl 6-oxa-2-azabicyclo[3.2.1]octane-2-carboxylate (2.40 g, 1.0 equiv) and HCl•MeOH (4 M, 4.85 mL, 2.0 equiv) in MeOH (20 mL) was added Pd/C (500 mg, 10% purity, 1.0 equiv). The reaction was degassed and purged with H₂ 3 times. The reaction was stirred under H₂ (15 psi) at 20° C. for 12 hours. The mixture was filtered and the filter was concentrated to afford the title compound (1.20 g, crude, HCl) as yellow solid.

Step B and C were according to Example 233 (step B and C) except that HCl was used instead of TFA in the last step. The title compound was obtained as yellow solid; ¹H NMR (400 MHz, METHANOL-d₄) δ=7.51 (dd, J=6.0, 8.8 Hz, 1H), 7.14 (t, J=9.6 Hz, 1H), 7.01-6.89 (m, 2H), 6.65-6.55 (m, 1H), 5.51-5.29 (m, 1H), 5.27-5.15 (m, 1H), 5.11-4.91 (m, 2H), 4.69-4.48 (m, 4H), 4.46-4.12 (m, 2H), 4.11-3.85 (m, 6H), 3.71-3.64 (m, 1H), 3.63-3.41 (m, 2H), 3.38 (td, J=3.6, 7.2 Hz, 1H), 3.26-3.12 (m, 5H), 2.98 (dt, J=5.2, 9.2 Hz, 1H), 2.71 (br d, J=14.4 Hz, 1H), 2.38-2.17 (m, 2H), 2.16-2.03 (m, 3H), 2.03-1.83 (m, 5H), 1.83-1.59 (m, 2H), 1.10 (q, J=7.2 Hz, 3H); LCMS (ESI, M+1): m/z=755.5.

### Example 625

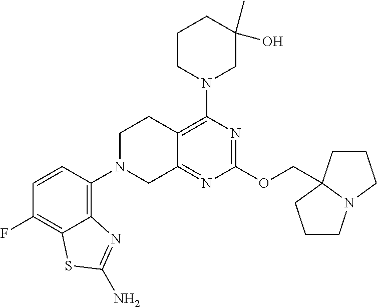 [SMILES: CC1(O)CCCN(c2nc(OCC34CCCN3CCC4)nc3c2CCN(c2ccc(F)c4sc(N)nc24)C3)C1]

### 1-(7-(2-amino-7-fluorobenzo[d]thiazol-4-yl)-2-((tetrahydro-1H-pyrrolizin-7a(5H)-yl)methoxy)-5,6,7,8-tetrahydropyrido[3,4-d]pyrimidin-4-yl)-3-methylpiperidin-3-ol

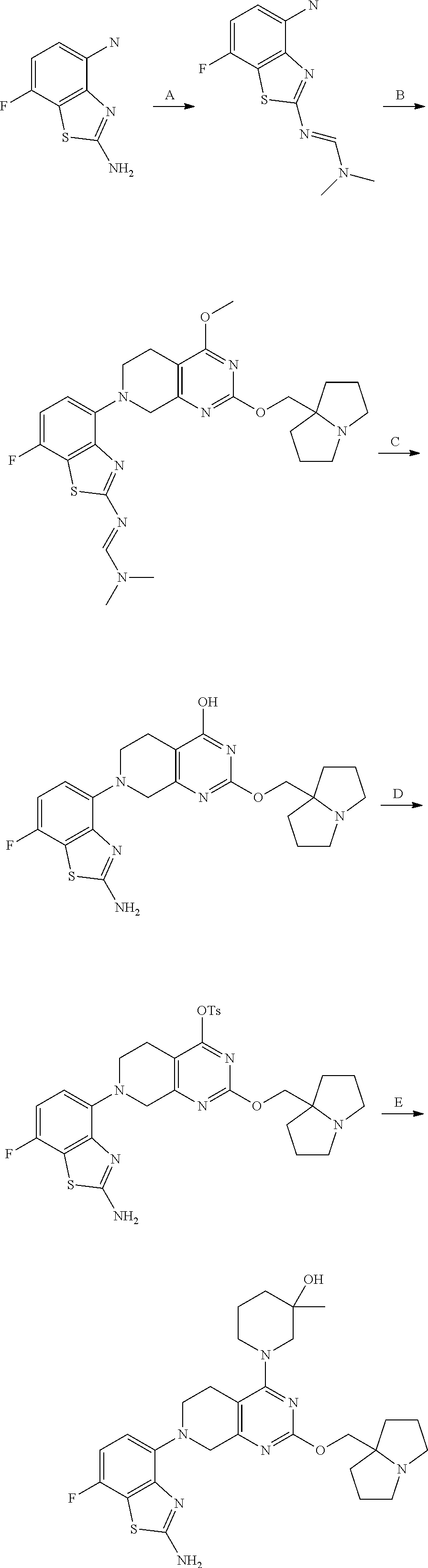

Step A. (E)-N′-(4-bromo-7-fluorobenzo[d]thiazol-2-yl)-N,N-dimethylformimidamide: To a solution of 4-bromo-7-fluorobenzo[d]thiazol-2-amine (5.00 g, 1.0 equiv) in DMF-DMA (10 mL) was stirred at 100° C. for 12 hours. The reaction mixture was triturated with petroleum ether (100 mL) to afford the title compound (4.70 g, 76% yield) as brown solid; LCMS (ESI, M+1): m/z=302.1.

Step B. (E)-N′-(7-fluoro-4-(2-((hexahydro-1H-pyrrolizin-7a-yl)methoxy)-4-methoxy-5,6-dihydropyrido[3,4-d] pyrimidin-7(8H)-yl)benzo[d]thiazol-2-yl)-N,N-dimethylformimidamide: To a solution of 2-((hexahydro-1H-pyrrolizin-7a-yl)methoxy)-4-methoxy-5,6,7,8-tetrahydropyrido[3,4-d]pyrimidine (1.50 g, 1.0 equiv) and (E)-N′-(4-bromo-7-fluorobenzo[d]thiazol-2-yl)-N,N-dimethylformimidamide (2.23 g, 1.5 equiv) in dioxane (15 mL) were added Cs₂CO₃ (4.82 g, 3.0 equiv) and CPhos Pd G3 (397 mg, 0.1 equiv). The reaction was degassed and purged with nitrogen 3 times. The reaction was stirred at 90° C. for 12 hours. The mixture was quenched with H₂O (50 mL) and extracted with EtOAc (3×100 mL). The combined organic layers were dried over anhydrous sodium sulfate, concentrated and purified by reversed phase flash chromatography [C18, 0.1% formic acid condition] to afford the title compound (1.40 g, 54% yield) as yellow solid; LCMS (ESI, M+1): m/z=526.1.

Step C. 7-(2-amino-7-fluorobenzo[d]thiazol-4-yl)-2-((hexahydro-1H-pyrrolizin-7a-yl)methoxy)-5,6,7,8-tetrahydropyrido[3,4-d] pyrimidin-4-ol: To a solution of EtSH (1.07 g, 9.0 equiv) in DMAc (10 mL) was added NaH (456 mg, 60% purity, 6.0 equiv) at 0° C. The reaction was stirred at 20° C. for 1 hour. To the mixture was added a solution of (E)-N′-(7-fluoro-4-(2-((hexahydro-1H-pyrrolizin-7a-yl)methoxy)-4-methoxy-5,6-dihydropyrido[3,4-d]pyrimidin-7(8H)-yl)benzo[d]thiazol-2-yl)-N,N-dimethylformimidamide(1.00 g, 1.0 equiv) in DMAc (2 mL). The reaction was stirred at 80° C. for 12 hours. The mixture was quenched with H₂O (50 mL) and extracted with EtOAc (3×100 mL). The combined organic layers were dried over anhydrous sodium sulfate, concentrated and purified with reversed phase flash chromatography [C18, 0.1% formic acid condition] to afford the title compound (140 mg, 14% yield) as yellow solid; LCMS (ESI, M+1): m/z=457.1.

Step D. 7-(2-amino-7-fluorobenzo[d]thiazol-4-yl)-2-((hexahydro-1H-pyrrolizin-7a-yl)methoxy)-5,6,7,8-tetrahydropyrido[3,4-d]pyrimidin-4-yl 4-methylbenzenesulfonate: To a mixture of 7-(2-amino-7-fluorobenzo[d]thiazol-4-yl)-2-((hexahydro-1H-pyrrolizin-7a-yl)methoxy)-5,6,7,8-tetrahydropyrido[3,4-d]pyrimidin-4-ol (50 mg, 1.0 equiv) and 4-methylbenzene-1-sulfonyl chloride (31.3 mg, 1.5 equiv) in DCM (1.0 mL) were added DIEA(42.5 mg, 3.0 equiv) and DMAP (1.32 mg, 0.1 equiv). The reaction was stirred at 25° C. for 12 hours. The mixture was quenched with H₂O (5 mL) and extracted with EtOAc (3×20 mL). The combined organic layers were dried over anhydrous sodium sulfate, concentrated to afford the title compound (55.0 mg, 76% yield) as yellow solid; LCMS (ESI, M+1): m/z=611.1.

Step E. 1-(7-(2-amino-7-fluorobenzo[d]thiazol-4-yl)-2-((hexahydro-1H-pyrrolizin-7a-yl)methoxy)-5,6,7,8-tetrahydropyrido[3,4-d]pyrimidin-4-yl)-3-methylpiperidin-3-ol: To a solution of 7-(2-amino-7-fluorobenzo[d]thiazol-4-yl)-2-((hexahydro-1H-pyrrolizin-7a-yl)methoxy)-5,6,7,8-tetrahydropyrido[3,4-d]pyrimidin-4-yl 4-methylbenzenesulfonate (200 mg, 1.0 equiv) and 3-methylpiperidin-3-ol (113 mg, 3.0 equiv) in DMF (0.5 mL) was added DIEA (127 mg, 3.0 equiv). The reaction was stirred at 25° C. for 12 hours. The mixture was concentrated and purified with prep-HPLC [Waters Xbridge C18 150×25 mm×5 μm; A: water (NH₄HCO₃), B: ACN, B %: 39%-69% over 8 min] to afford the title compound (26.1 mg, 14% yield) as off-white solid; ¹H NMR (400 MHz, METHANOL-d₄) δ=6.88 (br dd, J=3.6, 5.1 Hz, 1H), 6.85-6.75 (m, 1H), 4.23 (br d, J=8.1 Hz, 2H), 4.13 (s, 2H), 3.80-3.70 (m, 1H), 3.64 (br d, J=13.3 Hz, 1H), 3.49 (br d, J=6.0 Hz, 1H), 3.42 (br d, J=5.1 Hz, 1H), 3.36-3.34 (m, 1H), 3.31 (br s, 1H), 3.14-3.03 (m, 2H), 2.99-2.80 (m, 2H), 2.77-2.66 (m, 2H), 2.09-1.81 (m, 7H), 1.79-1.64 (m, 5H), 1.24 (s, 3H); LCMS (ESI, M+1): m/z=554.2.

### Example 626

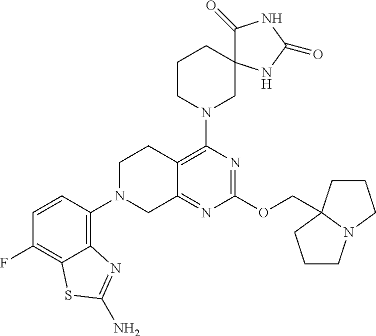 [SMILES: Nc1nc2c(N3CCc4c(nc(OCC56CCCN5CCC6)nc4N4CCCC5(C4)NC(=O)NC5=O)C3)ccc(F)c2s1]

### 7-(7-(2-amino-7-fluorobenzo[d]thiazol-4-yl)-2-((tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione

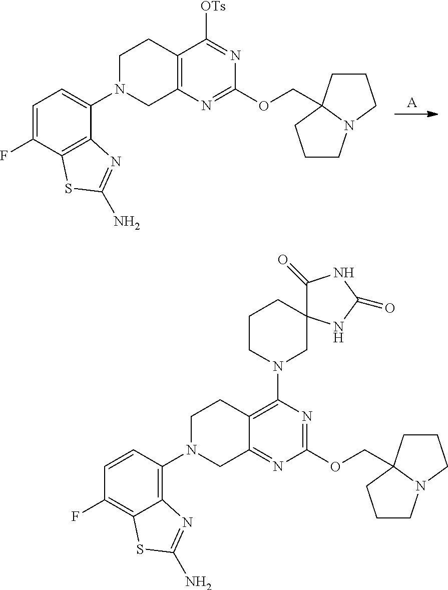

Step A. 7-(7-(2-amino-7-fluorobenzo[d]thiazol-4-yl)-2-((hexahydro-1H-pyrrolizin-7a-yl)methoxy)-5,6,7,8-tetrahydropyrido[3,4-d]pyrimidin-4-yl)-1,3,7-triazaspiro[4.5]decane-2,4-dione: To a solution of 7-(2-amino-7-fluorobenzo[d]thiazol-4-yl)-2-((hexahydro-1H-pyrrolizin-7a-yl)methoxy)-5,6,7,8-tetrahydropyrido[3,4-d]pyrimidin-4-yl 4-methylbenzenesulfonate (55.0 mg, 1.0 equiv) and 1,3,7-triazaspiro[4.5]decane-2,4-dione (30.5 mg, 2.0 equiv) in DMF (0.5 mL) was added DIEA (34.9 mg, 3.0 equiv). The reaction was stirred at 25° C. for 12 hours. The mixture was concentrated and purified with prep-HPLC [Phenomenex luna C18 150×25 mm×10 μm; A: water (FA), B: ACN, B %: 11%-41% over 10 min] to afford the title compound (18.1 mg, FA salt) as white solid; ¹H NMR (400 MHz, METHANOL-d₄) δ=8.49-8.45 (m, 1H), 6.91-6.84 (m, 1H), 6.83-6.76 (m, 1H), 4.52-4.40 (m, 2H), 4.34-4.13 (m, 3H), 4.09-3.99 (m, 1H), 3.71-3.62 (m, 2H), 3.51-3.37 (m, 3H), 3.28-3.18 (m, 3H), 3.01-2.84 (m, 2H), 2.34-2.05 (m, 9H), 1.96-1.82 (m, 3H); LCMS (ESI, M+1): m/z=608.1.

### Example 627

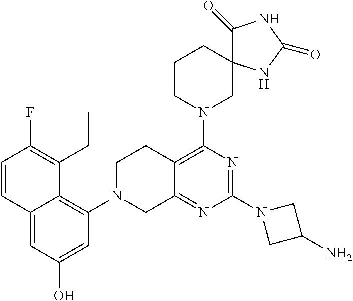 [SMILES: CCc1c(F)ccc2cc(O)cc(N3CCc4c(nc(N5CC(N)C5)nc4N4CCCC5(C4)NC(=O)NC5=O)C3)c12]

### 7-(2-(3-aminoazetidin-1-yl)-7-(8-ethyl-7-fluoro-3-hydroxynaphthalen-1-yl)-5,6,7,8-tetrahydropyrido[3,4-d]pyrimidin-4-yl)-1,3,7-triazaspiro[4.5]decane-2,4-dione

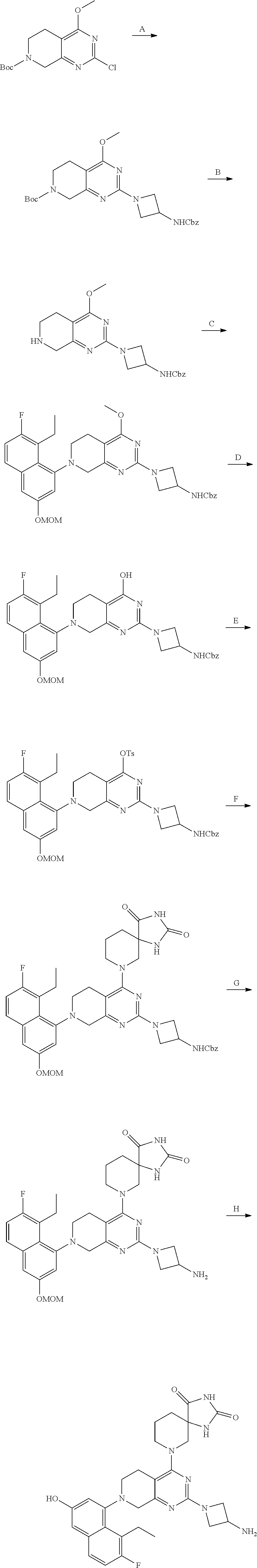

Step A. tert-butyl 2-(3-(((benzyloxy)carbonyl)amino)azetidin-1-yl)-4-methoxy-5,6-dihydropyrido[3,4-d]pyrimidine-7(8H)-carboxylate: To a mixture of tert-butyl 2-chloro-4-methoxy-5,6-dihydropyrido[3,4-d]pyrimidine-7(8H)-carboxylate (2.91 g, 1.0 equiv), benzyl azetidin-3-ylcarbamate (3.00 g, 1.5 equiv), (R)-(+)-2,2′-bis(diphenylphosphino)-1,1′-binaphthyl (845 mg, 0.20 equiv) and cesium carbonate (6.64 g, 3.0 equiv) in methylbenzene (30 mL) was added palladium acetate (152 mg, 0.10 equiv). The reaction was degassed and purged with nitrogen 3 times. The reaction was stirred at 100° C. for 2 hours. The mixture was diluted with dichloromethane (40 mL) and methanol (10 mL), filtered and concentrated. The residue was purified with column chromatography (silicon dioxide, Petroleum ether/Ethyl acetate=10/1 to 1/1) and reversed-phase HPLC (0.1% FA condition) to afford the title compound (1.50 g, 45% yield) as yellow solid. LCMS (ESI, M+1): m/z=470.2.

Step B. benzyl (1-(4-methoxy-5,6,7,8-tetrahydropyrido[3,4-d]pyrimidin-2-yl)azetidin-3-yl)carbamate: A mixture of tert-butyl 2-(3-(((benzyloxy)carbonyl)amino)azetidin-1-yl)-4-methoxy-5,6-dihydropyrido[3,4-d]pyrimidine-7(8H)-carboxylate (1.40 g, 1.0 equiv) in HCl•MeOH (4 M, 10 mL, 13 equiv) was stirred at 20° C. for 1 hour. The mixture was concentrated under vacuum. The residue was diluted with water (10 mL). The mixture was adjusted pH to 11 with NaOH solution. The mixture was extracted with EtOAc (3×50 mL). The combined organic layers were washed with brine (50 mL), dried over anhydrous sodium sulfate, concentrated to afford the title compound (0.91 g, 79% yield) as white solid. LCMS (ESI, M+1): m/z=370.1.

Step C. benzyl (1-(7-(8-ethyl-7-fluoro-3-(methoxymethoxy)naphthalen-1-yl)-⁴-methoxy-5,6,7,8-tetrahydropyrido[3,4-d] pyrimidin-2-yl)azetidin-3-yl)carbamate: To a mixture of benzyl (1-(4-methoxy-5,6,7,8-tetrahydropyrido[3,4-d]pyrimidin-2-yl)azetidin-3-yl)carbamate (0.91 g, 1.0 equiv), 8-ethyl-7-fluoro-3-(methoxymethoxy)naphthalen-1-yl trifluoromethanesulfonate (1.88 g, 2.0 equiv), Xantphos (285 mg, 0.20 equiv) and cesium carbonate (2.41 g, 3.0 equiv) in methylbenzene was added tris(dibenzylideneacetone)dipalladium(0) (226 mg, 0.10 equiv). The reaction was degassed and purged with nitrogen 3 times. The reaction was stirred at 100° C. for 16 hours. The mixture was diluted with dichloromethane (20 mL) and methanol (10 mL). The mixture was filtered and purified with column chromatography (silicon dioxide, Petroleum ether/Ethyl acetate=10/1 to 3/1) to afford the title compound (0.5 g, 32% yield) as white solid. LCMS (ESI, M+1): m/z=602.3.

Step D. benzyl (1-(7-(8-ethyl-7-fluoro-3-(methoxymethoxy)naphthalen-1-yl)-4-hydroxy-5,6,7,8-tetrahydropyrido[3,4-d]pyrimidin-2-yl)azetidin-3-yl)carbamate: To a solution of ethanethiol (310 mg, 6.0 equiv) in dimethylacetamide (2 mL) was added NaH (100 mg, 3.0 equiv, 60% purity). The reaction was stirred at 0° C. for 0.5 hour. A solution of benzyl (1-(7-(8-ethyl-7-fluoro-3-(methoxymethoxy)naphthalen-1-yl)-4-methoxy-5,6,7,8-tetrahydropyrido[3,4-d]pyrimidin-2-yl)azetidin-3-yl)carbamate (0.5 g, 1.0 equiv) in dimethylacetamide (3 mL) was added into the mixture. The reaction was stirred at 60° C. for 0.5 hours. The mixture was poured with water (10 mL) and extracted with EtOAc (2×20 mL). The combined organic layers were washed with brine (30 mL), dried with anhydrous sodium sulfate, concentrated to afford the title compound (0.500 g, 32% yield) as black oil and used into next step with further purification. LCMS (ESI, M+1): m/z=588.6.

Step E. 2-(3-(((benzyloxy)carbonyl)amino)azetidin-1-yl)-7-(8-ethyl-7-fluoro-3-(methoxymethoxy)naphthalen-1-yl)-5,6,7,8-tetrahydropyrido[3,4-d]pyrimidin-4-yl 4-methylbenzenesulfonate: To a solution of benzyl (1-(7-(8-ethyl-7-fluoro-3-(methoxymethoxy)naphthalen-1-yl)-4-hydroxy-5,6,7,8-tetrahydropyrido[3,4-d]pyrimidin-2-yl)azetidin-3-yl)carbamate (200 mg, 1.0 equiv) and 4-methylbenzenesulfonyl chloride (71.4 mg, 1.1 equiv) in dichloromethane (2.0 mL) were added diisopropylethylamine (132 mg, 3.0 equiv) and DMAP (4.16 mg, 0.10 equiv). The reaction was stirred at 25° C. for 1 hour. The mixture was concentrated and purified with column chromatography (silicon dioxide, Petroleum ether/Ethyl acetate=5/1 to 1/1) to afford the title compound (170 mg, 64% yield) as orange solid. LCMS (ESI, M+1): m/z=742.7.

Step F. benzyl (1-(4-(2,4-dioxo-1,3,7-triazaspiro[4.5]decan-7-yl)-7-(8-ethyl-7-fluoro-3-(methoxymethoxy)naphthalen-1-yl)-5,6,7,8-tetrahydropyrido[3,4-d]pyrimidin-2-yl)azetidin-3-yl)carbamate: To a mixture of 2-(3-(((benzyloxy)carbonyl)amino)azetidin-1-yl)-7-(8-ethyl-7-fluoro-3-(methoxymethoxy)naphthalen-1-yl)-⁵,6,7,8-tetrahydropyrido[3,4-d] pyrimidin-4-yl 4-methylbenzenesulfonate (830 mg, 1.0 equiv) and 1,3,7-triazaspiro[4.5]decane-2,4-dione (378 mg, 2.0 equiv) in DMF (3.0 mL) were added DIEA (868 mg, 6.0 equiv) and 4 Å molecular sieve (10.0 mg, 1.0 equiv). The reaction was stirred at 60° C. for 12 hours. The mixture was filtered and purified by reversed phase flash [water (FA, 0.10%)/acetonitrile] to afford the title compound (260 mg, 30% yield) as white oil; LCMS (ESI, M+1): m/z=739.5.

Step G. 7-(2-(3-aminoazetidin-1-yl)-7-(8-ethyl-7-fluoro-3-(methoxymethoxy)naphthalen-1--yl)-5,6,7,8-tetrahydropyrido[3,4-d]pyrimidin-4-yl)-1,3,7-triazaspiro[4.5]decane-2,4-dione: To a mixture of benzyl (1-(4-(2,4-dioxo-1,3,7-triazaspiro[4.5]decan-7-yl)-7-(8-ethyl-7-fluoro-3-(methoxymethoxy)naphthalen-1-yl)-5,6,7,8-tetrahydropyrido[3,4-d]pyrimidin-2-yl)azetidin-3-yl)carbamate (250 mg, 1.0 equiv) in MeOH (1.5 mL) and H₂O (1.5 mL) was added LiOH•H₂O (710 mg, 50 equiv). The reaction was stirred at 90° C. for 2 hours. The mixture was diluted with water (15 mL) and extracted with EtOAc (10 mL×3). The combined organic layers were dried over anhydrous sodium sulfate and concentrated to afford the title compound (140 mg, 53% yield) as brown solid; LCMS (ESI, M+1): m/z=605.4.

Step H. 7-(2-(3-aminoazetidin-1-yl)-7-(8-ethyl-7-fluoro-3-hydroxynaphthalen-1-yl)-5,6,7,8-tetrahydropyrido[3,4-d]pyrimidin-4-yl)-1,3,7-triazaspiro[4.5]decane-2,4-dione: To a mixture of 7-(2-(3-aminoazetidin-1-yl)-7-(8-ethyl-7-fluoro-3-(methoxymethoxy)naphthalen-1-yl)-5,6,7,8-tetrahydropyrido[3,4-d]pyrimidin-4-yl)-1,3,7-triazaspiro[4.5]decane-2,4-dione (100 mg, 1.0 equiv) in HCl-MeOH (4 M, 4.0 mL, 97 equiv). The reaction was stirred at 25° C. for 0.5 hour. The mixture was concentrated. The residue was diluted with water (1 mL). The mixture was adjusted to pH=7 with saturated NaHCO₃ (5 mL) and extracted with ethyl acetate (2×5 mL). The combined organic layers were dried over anhydrous sodium sulfate, concentrated, and purified with prep-HPLC (column: Welch Ultimate C18 150*25 mm*5 μm;mobile phase: [water(FA)-ACN];B %: 8%-38%,10 min) to afford the title compound (15.5 mg, 16% yield, HCOOH) as orange solid; ¹H NMR (400 MHz, METHANOL-d₄) δ=7.59-7.44 (m, 1H), 7.14 (t, J=9.2 Hz, 1H), 7.05-6.84 (m, 2H), 4.44-4.25 (m, 2H), 4.18-3.95 (m, 4H), 3.94-3.76 (m, 2H), 3.61 (s, 1H), 3.48 (br dd, J=1.6, 3.2 Hz, 2H), 3.41-3.34 (m, 1H), 3.30-2.84 (m, 4H), 2.79 (s, 1H), 1.96 (br s, 2H), 1.88 (br s, 2H), 1.17-1.03 (m, 3H). LCMS (ESI, M+1): m/z=561.6.

### Example 628

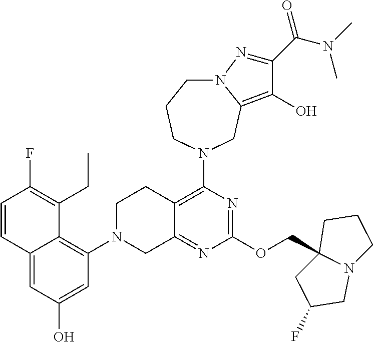 [SMILES: CCc1c(F)ccc2cc(O)cc(N3CCc4c(nc(OC[C@@]56CCCN5C[C@H](F)C6)nc4N4CCCn5nc(C(=O)N(C)C)c(O)c5C4)C3)c12]

### 5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-3-hydroxy-N,N-dimethyl-5,6,7,8-tetrahydro-4H-pyrazolo[1,5-a][1,4]diazepine-2-carboxamide

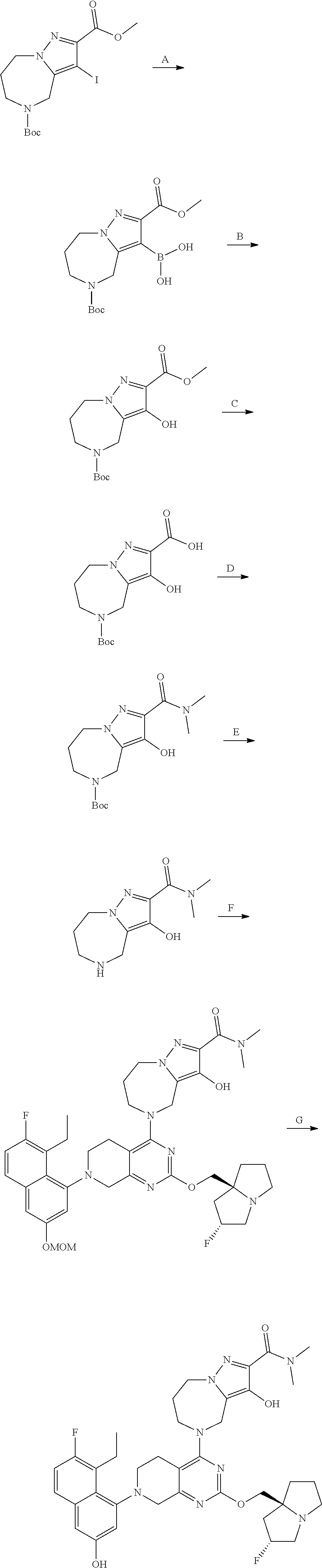

Step A. (5-(tert-butoxycarbonyl)-2-(methoxycarbonyl)-5,6,7,8-tetrahydro-4H-pyrazolo[1,5-a][1,4]diazepin-3-yl)boronic acid. To a mixture of 5-tert-butyl 2-methyl 3-iodo-7,8-dihydro-4H-pyrazolo[1,5-a][1,4]diazepine-2,5(6H)-dicarboxylate (1.40 g, 1.0 equiv) and 4,4,5,5-tetramethyl-1,3,2-dioxaborolane (4.25 g, 10 equiv) in MeCN (14 mL) were added cis-dichloro-1,1′-bis(diphenylphosphino)ferrocene palladium(II) (486 mg, 0.2 equiv) and TEA (1.01 g, 3.0 equiv). The reaction was degassed and purged with nitrogen 3 times. The reaction was stirred at 80° C. for 2 hours. The mixture was concentrated and purified by reversed phase flash [water (FA, 0.1%)/acetonitrile] to afford the title compound (0.66 g, 53% yield) as brown oil; LCMS (ESI, M+1): m/z=339.9.

Step B. 5-tert-butyl 2-methyl 3-hydroxy-7,8-dihydro-4H-pyrazolo[1,5-a][1,4]diazepine-2,5(6H)-dicarboxylate: To a mixture of (5-(tert-butoxycarbonyl)-2-(methoxycarbonyl)-5,6,7,8-tetrahydro-4H-pyrazolo[1,5-a][1,4]diazepin-3-yl)boronic acid (600 mg, 1.0 equiv) in H₂O (2 mL) and THE (6 mL) were added H₂O₂ (1.73 g, 30% purity, 8.6 equiv) and NH₄CO₃ (280 mg, 2.0 equiv). The reaction was stirred at 25° C. for 12 hours. The mixture was quenched with Na₂SO₃ (2 mL) and extracted with DCM (5 mL×3). The combined organic layers were dried over anhydrous sodium sulfate and concentrated to afford the title compound (440 mg, 77% yield) as brown solid;

Step C. 5-(tert-butoxycarbonyl)-3-hydroxy-5,6,7,8-tetrahydro-4H-pyrazolo[1,5-a][1,4]diazepine-2-carboxylic acid: To a mixture of 5-tert-butyl 2-methyl 3-hydroxy-7,8-dihydro-4H-pyrazolo[1,5-a][1,4]diazepine-2,5(6H)-dicarboxylate (250 mg, 1.0 equiv) in MeOH (3 mL) was added LiOH•H₂O (1.68 g, 50 equiv). The reaction was stirred at 25° C. for 12 hours. The mixture was diluted with water (5 mL) and concentrated under vacuum to remove MeOH. The residue was diluted with water (5 mL). The pH of the residue was adjusted to 4 by adding HCl dropwise (2 M) and extracted with ethyl acetate (2×10 mL). The combined organic layers were dried over anhydrous sodium sulfate and concentrated to afford the title compound (290 mg, crude) as brown solid; LCMS (ESI, M+1): m/z=298.1.

Step D. tert-butyl 2-(dimethylcarbamoyl)-3-hydroxy-7,8-dihydro-4H-pyrazolo[1,5-a][1,4]diazepine-5(6H)-carboxylate: To a mixture of 5-(tert-butoxycarbonyl)-3-hydroxy-5,6,7,8-tetrahydro-4H-pyrazolo[1,5-a][1,4]diazepine-2-carboxylic acid (50.0 mg, 1.0 equiv) and dimethylamine (2 M, 2.0 equiv) in DMF (0.5 mL) were added HATU (192 mg, 3.0 equiv) and DIEA (217 mg, 10 equiv). The reaction was stirred at 25° C. for 2 hours. The mixture was filtered and purified by reversed phase flash [water (FA, 0.1%)/acetonitrile] to afford the title compound (27 mg, 48% yield) as yellow solid; LCMS (ESI, M+1): m/z=325.0.

Step E. 3-hydroxy-N,N-dimethyl-5,6,7,8-tetrahydro-4H-pyrazolo[1,5-a][1,4]diazepine-2-carboxamide: To a mixture of tert-butyl 2-(dimethylcarbamoyl)-3-hydroxy-7,8-dihydro-4H-pyrazolo[1,5-a][1,4]diazepine-5(6H)-carboxylate (75.0 mg, 1.0 equiv) in ACN (1.0 mL) was added HCl•dioxane (4 M, 1.0 mL, 17 equiv). The reaction was stirred 25° C. for 1 hour. The mixture was concentrated to afford the title compound (90.0 mg, HCl) as yellow oil.

Step F. 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-3-hydroxy-N,N-dimethyl-5,6,7,8-tetrahydro-4H-pyrazolo[1,5-a][1,4]diazepine-2-carboxamide: To a mixture of 7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-ol (120 mg, 1.0 equiv) and 3-hydroxy-N,N-dimethyl-5,6,7,8-tetrahydro-4H-pyrazolo[1,5-a][1,4]diazepine-2-carboxamide (77.5 mg 1.7 equiv, HCl) in DMF (0.5 mL) were added DIEA (335 mg, 15 equiv) and 4 Å molecular sieve (10.0 mg, 1.0 equiv). The reaction was stirred 60° C. for 12 hours. The mixture was filtered and purified by reversed phase flash [water (FA, 0.1%)/acetonitrile] to afford the title compound (77 mg, 58% yield) as brown solid. LCMS (ESI, M+1): m/z=747.6.

Step G. 5-(7-(8-ethyl-7-fluoro-3-hydroxynaphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-3-hydroxy-N,N-dimethyl-5,6,7,8-tetrahydro-4H-pyrazolo[1,5-a][1,4]diazepine-2-carboxamide: A mixture of 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl)-3-hydroxy-N,N-dimethyl-5,6,7,8-tetrahydro-4H-pyrazolo[1,5-a][1,4]diazepine-2-carboxamide (70.0 mg, 1.0 equiv) in HCl-MeOH (4 M, 1.0 mL, 43 equiv) was stirred at 25° C. for 0.5 hour. The mixture was concentrated. The residue was diluted with water (1.0 mL). The mixture was adjusted to pH=7 with saturated NaHCO₃ (2 mL) and extracted with ethyl acetate (2×2 mL). The combined organic layers were dried over anhydrous sodium sulfate, concentrated, and purified with prep-HPLC (column: Phenomenex luna C18 150*25 mm*10 μm; mobile phase: [water (FA)-ACN]; B %: 12%-42%, 10 min) to afford the title compound (34.1 mg, 49% yield, 0.23 HCOOH) as orange solid. ¹H NMR (400 MHz, METHANOL-d₄) δ=7.50 (dd, J=5.6, 9.0 Hz, 1H), 7.14 (t, J=9.6 Hz, 1H), 7.03-6.91 (m, 2H), 5.43-5.15 (m, 1H), 4.96 (br d, J=2.4 Hz, 1H), 4.73 (br s, 1H), 4.46 (br t, J=5.2 Hz, 2H), 4.23-4.09 (m, 3H), 4.05 (br d, J=17.6 Hz, 1H), 4.01-3.91 (m, 1H), 3.64 (br d, J=17.6 Hz, 1H), 3.55-3.34 (m, 7H), 3.27-3.12 (m, 4H), 3.11-2.93 (m, 4H), 2.74 (br d, J=15.2 Hz, 1H), 2.41-2.13 (m, 3H), 2.12-1.82 (m, 5H), 1.09 (dt, J=1.6, 7.2 Hz, 3H); LCMS (ESI, M+1): m/z=703.6.

### Example 629

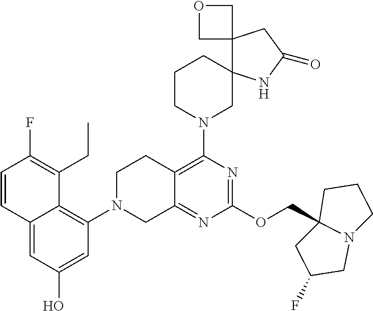 [SMILES: CCc1c(F)ccc2cc(O)cc(N3CCc4c(nc(OC[C@@]56CCCN5C[C@H](F)C6)nc4N4CCCC5(C4)NC(=O)CC54COC4)C3)c12]

### 7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-2-oxa-7,11-diazadispiro[3.0.55.34]tridecan-12-one

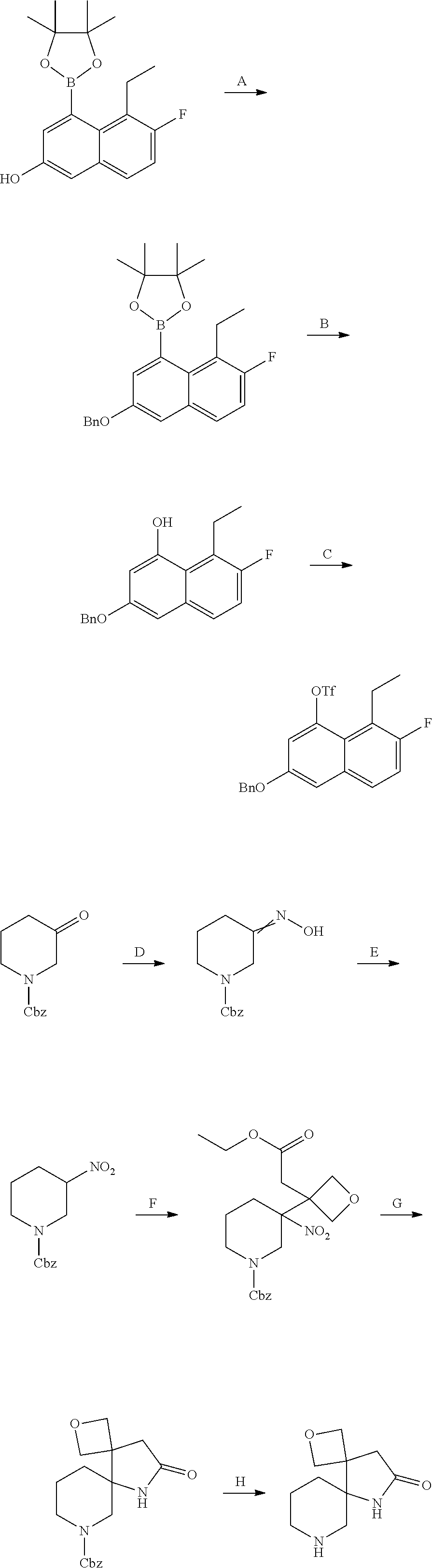

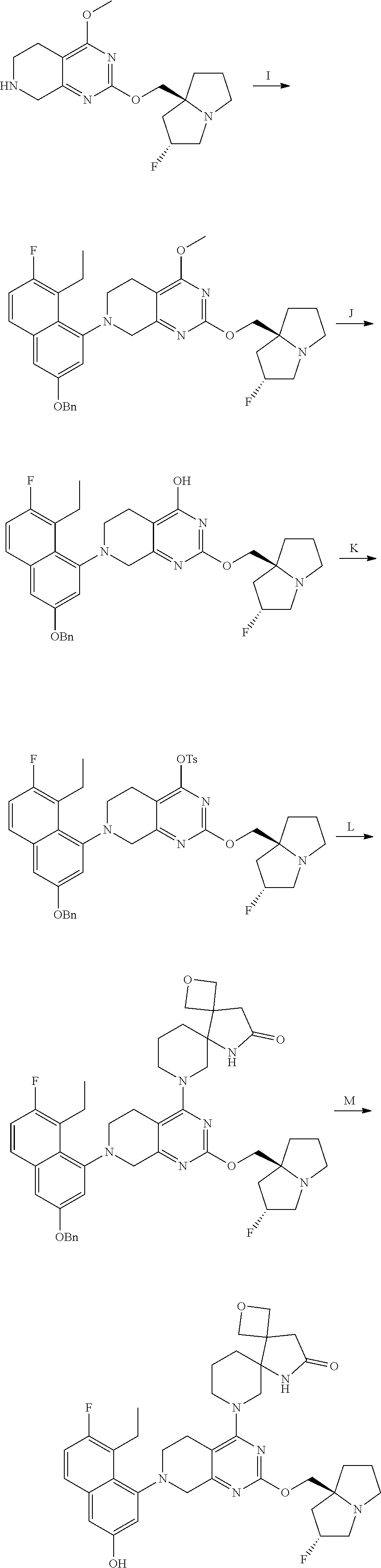

Step A. 2-(3-(benzyloxy)-8-ethyl -7-fluoronaphthalen-1-yl)-4,4,5,5-tetramethyl-1,3,2-dioxaborolane: To a mixture of 5-ethyl-6-fluoro-4-(4,4,5,5-tetramethyl-1,3,2-dioxaborolan-2-yl)naphthalen-2-ol (30.0 g, 1.0 equiv) and BnBr (17.9 g, 1.1 equiv) in MeCN (300 mL) was added K₂CO₃ (39.3 g, 3.0 equiv). The reaction was stirred at 90° C. for 2 hours. The mixture was concentrated under vacuum to remove MeCN. The residue was diluted with water (200 mL) and extracted with EtOAc (2×200 mL). The combined organic layers were washed with brine (200 mL), dried over anhydrous sodium sulfate, concentrated. The residue was washed with MeCN (100 mL), dried over vacuum to to afford the title compound (38.0 g, 86% yield) as orange solid;

Step B. 3-(benzyloxy)-8-ethyl-7-fluoronaphthalen-1-ol: To a mixture of 2-(3-(benzyloxy)-8-ethyl-7-fluoronaphthalen-1-yl)-4,4,5,5-tetramethyl-1,3,2-dioxaborolane (38.0 g, 1.0 equiv) and NaHCO₃ (39.3 g, 5.0 equiv) in THE (600 mL) and H₂O (300 mL) was added dropwise H₂O₂ (156 g, 30% purity, 15 equiv) at 0° C. in 1 hour. The reaction was stirred at 20° C. for 1 hour. The mixture was quenched with saturated Na₂SO₃ (300 mL) and extracted with ethyl acetate (2×400 mL). The combined organic layer was washed with brine (300 mL), dried over anhydrous sodium sulfate, concentrated to afford the title compound (29.0 g, crude) as yellow solid; LCMS (ESI, M+1): m/z=297.0.

Step C. 3-(benzyloxy)-8-ethyl-7-fluoronaphthalen-1-yl trifluoromethanesulfonate: To a solution of 3-(benzyloxy)-8-ethyl-7-fluoronaphthalen-1-ol (29.0 g, 1.0 equiv) and DIEA (37.9 g, 3.0 equiv) in DCM (300 mL) was added Trifluoroacetyl Trifluoromethanesulfonate (41.4 g, 1.5 equiv). The mixture was stirred at −40° C. for 0.5 hours. The mixture was diluted with water (200 mL) and extracted with DCM (3×100 mL). The combined organic layers were washed with brine (100 mL), dried over anhydrous sodium sulfate, concentrated, and purified with column chromatography (SiO₂, petroleum ether/ethyl acetate=40/1 to 1/1) and reversed phase flash (0.1% FA condition) to afford the title compound (37.0 g, 87% yield) as yellow oil; ¹H NMR (400 MHz, CHLOROFORM-d) δ=7.44 (dd, J=5.6, 8.8 Hz, 1H), 7.39-7.03 (m, 8H), 5.02 (s, 2H), 3.13 (dq, J=2.8, 7.6 Hz, 2H), 1.13 (t, J=7.6 Hz, 3H).

Step D. benzyl 3-(hydroxyimino)piperidine-1-carboxylate: To a solution of benzyl 3-oxopiperidine-1-carboxylate (10.0 g, 1.0 equiv) and KOAc (12.6 g, 3.0 equiv) in MeOH (100 mL) and H₂O (35 mL) was added NH₂OH•HCl (8.94 g, 3.0 equiv). The reaction was stirred at 60° C. for 1 hour. The mixture was concentrated to remove MeOH. The residue was extracted with ethyl acetate (3×200 mL). The organic layers were dried over anhydrous sodium sulfate, concentrated, and purified with reversed phase flash (C18, 0.1% formic acid condition) to afford the title compound (10.4 g, 98% yield) as yellow oil; ¹H NMR (400 MHz, CHLOROFORM-d) 6=8.44-8.02 (m, 1H), 7.43-7.28 (m, 5H), 5.18-5.08 (m, 2H), 4.44 (s, 1H), 4.11 (s, 1H), 3.61-3.51 (m, 2H), 2.68-2.35 (m, 2H), 1.86-1.71 (m, 2H); LCMS (ESI, M+1): m/z=249.3.

Step E. benzyl 3-nitropiperidine-1-carboxylate: To a solution of urea hydrogen peroxide (9.77 g, 2.87 equiv) in ACN (45 mL) was added a solution of TFAA (19.1 g, 12.6 mL, 2.50 equiv) in ACN (20 mL) dropwise at 0° C. The reaction was stirred at 0° C. for 1 hour. The resulting solution was added dropwise into a mixture of benzyl 3-(hydroxyimino)piperidine-1-carboxylate (9.0 g, 1.0 equiv) and NaHCO₃ (15.2 g, 7.05 mL, 5.0 equiv) in ACN (45 mL) at 80° C. The reaction was stirred at 80° C. for 1 hour. The mixture was quenched with saturated Na₂SO₃ aqueous solution (100 mL) and extracted with DCM (2×100 mL). The organic layers were dried over anhydrous sodium sulfate, concentrated, and purified with reversed phase flash (C18, 0.1% formic acid condition) to afford the title compound (5.30 g, 53% yield) as yellow oil.

Step F. benzyl 3-(3-(2-ethoxy-2-oxoethyl)oxetan-3-yl)-3-nitropiperidine-1-carboxylate: To a solution of benzyl 3-nitropiperidine-1-carboxylate (500 mg, 1.0 equiv) in dioxane (10 mL) were added ethyl 2-(oxetan-3-ylidene)acetate (538 mg, 2.0 equiv) and CsF (1.72 g, 6.0 equiv). The reaction was stirred at 90° C. for 12 hours. The mixture was diluted with H₂O (10 mL) and extracted with ethyl acetate (3×10 mL). The organic layer was dried over anhydrous sodium sulfate, concentrated and purified with reversed phase flash (C18, 0.1% formic acid condition) to afford the title compound (1.40 g, 43% yield) as yellow oil; LCMS (ESI, M+1): m/z=407.3.

Step G. benzyl 12-oxo-2-oxa-7,11-diazadispiro[3.0.55.34]tridecane-7-carboxylate: To a solution of benzyl 3-(3-(2-ethoxy-2-oxoethyl)oxetan-3-yl)-3-nitropiperidine-1-carboxylate (500 mg, 1.0 equiv) in AcOH (7 mL) was added Fe (344 mg, 5.0 equiv). The reaction was stirred at 65° C. for 12 hours. The mixture was diluted with H₂O (5 mL) and extracted with ethyl acetate (3×5 mL). The organic layer was dried over anhydrous sodium sulfate, concentrated and purified with reversed phase flash (C18, 0.1% formic acid condition) to afford the title compound (200 mg, 49% yield) as yellow oil; ¹H NMR (400 MHz, CHLOROFORM-d) δ=7.42-7.30 (m, 5H), 6.41 (br s, 1H), 5.24-5.03 (m, 2H), 4.90 (d, J=6.8 Hz, 2H), 4.40 (dd, J=4.0, 6.4 Hz, 2H), 4.25-4.14 (m, 1H), 4.07-3.88 (m, 1H), 3.13 (d, J=13.2 Hz, 1H), 2.96-2.81 (m, 3H), 1.89-1.75 (m, 3H), 1.67-1.58 (m, 1H); LCMS (ESI, M+1): m/z=331.1.

Step H. 2-oxa-7,11-diazadispiro[3.0.550.3⁴]tridecan-12-one: To a solution of benzyl 12-oxo-2-oxa-7,11-diazadispiror3.0.550.341tridecane-7-carboxylate (100 mg, 1.0 equiv) in MeOH (2 mL) was added Pd/C (20 mg, 10% purity) under N₂ atmosphere. The suspension was degassed under vacuum and purged with H₂ several times. The mixture was stirred under H₂ (15 Psi) at 20° C. for 3 hours. The mixture was filtered and concentrated to afford the title compound (70 mg, crude) as yellow oil.

Step I. 7-(3-(benzyloxy)-8-ethyl-7-fluoronaphthalen-1-yl)-2-(((2R,7aS)-2-fluorohexahydro-1H-pyrrolizin-7a-yl)methoxy)-4-methoxy-5,6,7,8-tetrahydropyrido[3,4-d]pyrimidine: To a mixture of 2-(((2R,7aS)-2-fluorohexahydro-1H-pyrrolizin-7a-yl)methoxy)-4-methoxy-5,6,7,8-tetrahydropyrido[3,4-d]pyrimidine (17.5 g, 1.0 equiv), Cs₂CO₃ (53.1 g, 3.0 equiv) and 3-(benzyloxy)-8-ethyl-7-fluoronaphthalen-1-yl trifluoromethanesulfonate (30.2 g, 1.3 equiv) in toluene (300 mL) were added Xantphos (6.28 g, 0.20 equiv) and Pd₂(dba)₃ (4.97 g, 0.1 equiv). The reaction was degassed and purged with nitrogen 3 times. The reaction was stirred at 110° C. for 12 hours. The mixture was diluted with water (300 mL) and extracted with ethyl acetate (2×300 mL). The combined organic layers were washed with brine (200 mL), dried over anhydrous sodium sulfate, concentrated and purified with by reversed-phase HPLC [water (FA, 0.1%)/acetonitrile] to afford the title compound (5.60 g, 16% yield) as brown solid; LCMS (ESI, M+1): m/z=601.3.

Step J. 7-(3-(benzyloxy)-8-ethyl-7-fluoronaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-ol: To a mixture of EtSH (2.12 g, 3.7 equiv) in DMAC (55 mL) was added NaH (915 mg, 60% purity, 2.5 equiv). The reaction was stirred at 0° C. for 0.5 hours. A solution of 7-(3-(benzyloxy)-8-ethyl-7-fluoronaphthalen-1-yl)-2-(((2R,7aS)-2-fluorohexahydro-1H-pyrrolizin-7a-yl)methoxy)-4-methoxy-5,6,7,8-tetrahydropyrido[3,4-d]pyrimidine (5.5 g, 1.0 equiv) in DMAc (20 mL) was added into above mixture. The reaction was stirred at 60° C. for 1 hour. The mixture was diluted with water (55 mL). The mixture was adjusted to pH=6 with 2M HCl aqueous and extracted with ethyl acetate (3×50 mL). The combined organic layers were washed with brine (2×50 mL), dried over anhydrous sodium sulfate, concentrated to afford the title compound (7.00 g, crude) as brown oil.

Step K. 7-(3-(benzyloxy)-8-ethyl-7-fluoronaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate: To a mixture of 7-(3-(benzyloxy)-8-ethyl-7-fluoronaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-ol (7 g, 1.0 equiv) and DIEA (4.63 g, 3.0 equiv) in DCM (70 mL) were added DMAP (145.8 mg, 0.1 equiv) and 4-methylbenzene-1-sulfonyl chloride (3.41 g, 1.5 equiv). The reaction was stirred at 0° C. for 1 hour. The mixture was concentrated and purified by reversed-phase HPLC [water (FA, 0.1%)/acetonitrile] to afford the title compound (4.80 g, 52% yield) as brown solid; LCMS (ESI, M+1): m/z=741.2

Step L. 7-(7-(3-benzyloxy-8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d] pyrimidin-4-yl)-2-oxa-7,11-diazadispiro[3.0.5³.3⁴]tridecan-12-one: To a solution of 7-(3-(benzyloxy)-8-ethyl-7-fluoronaphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (100 mg, 1.0 equiv), 4 Å molecular sieve (5 mg) and DIEA (87.2 mg, 5.0 equiv) in DMF (2 mL) was added 2-oxa-7,11-diazadispiro[3.0.550.34]tridecan-12-one (53.0 mg, 2.0 equiv). The reaction was stirred at 40° C. for 12 hours. The mixture was concentrated and purified by reversed-phase HPLC [water (FA, 0.1%)/acetonitrile] to afford the title compound (100 mg, 97% yield) as yellow oil; LCMS (ESI, M+1): m/z=765.7.

Step M. 7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-2-oxa-7,11-diazadispiro[3.0.5⁵0.3⁴]tridecan-12-one: To a solution of 7-[7-(3-benzyloxy-8-ethyl-7-fluoro-1-naphthyl)-2-[[(2R,8S)-2-fluoro-1,2,3,5,6,7-hexahydropyrrolizin-8-yl]methoxy]-6,8-dihydro-5H-pyrido[3,4-d]pyrimidin-4-yl]-2-oxa-7,11-diazadispiro[3.0.55.3⁴]tridecan-12-one (100 mg, 1.0 equiv) in MeOH (1 mL) was added Pd/C (20 mg, 10% purity) under N₂. The reaction was degassed and purged with H₂ 3 times. The reaction was stirred at 20° C. for 12 hours under H₂ (15 Psi). The mixture was concentrated and purified by prep-HPLC (column: Phenomenex Luna C18 150×25 mm×10 μm; mobile phase: [water(FA)-ACN]; B %: 16%-46%, 10 min) to afford the title compound (24.1 mg, 26% yield, 0.6 HCOOH) as yellow solid; ¹H NMR (400 MHz, METHANOL-d₄) δ=7.51 (dd, J=6.0, 8.8 Hz, 1H), 7.14 (t, J=9.2 Hz, 1H), 7.04-6.91 (m, 2H), 5.49-5.26 (m, 1H), 5.08-4.97 (m, 2H), 4.50 (d, J=6.8 Hz, 1H), 4.42-4.38 (m, 1H), 4.33-4.24 (m, 1H), 4.24-4.16 (m, 2H), 4.16-4.05 (m, 2H), 3.71-3.60 (m, 1H), 3.52-3.40 (m, 4H), 3.38-3.35 (m, 1H), 3.29 (br s, 1H), 3.20-3.11 (m, 3H), 3.11-3.02 (m, 1H), 3.02-2.89 (m, 2H), 2.82-2.68 (m, 1H), 2.49-2.26 (m, 2H), 2.23-1.96 (m, 6H), 1.95-1.77 (m, 3H), 1.15-1.06 (m, 3H); LCMS (ESI, M+1): m/z=675.1.

### Example 630

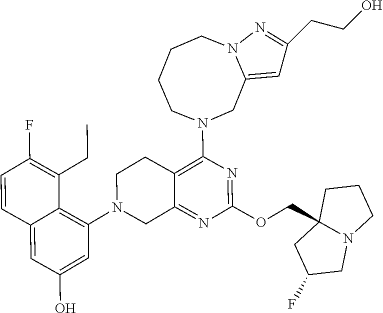 [SMILES: CCc1c(F)ccc2cc(O)cc(N3CCc4c(nc(OC[C@@]56CCCN5C[C@H](F)C6)nc4N4CCCCn5nc(CCO)cc5C4)C3)c12]

### 5-ethyl-6-fluoro-4-(2-(((2R,7aS)-2-fluorotetrahydro-1H-pyrrolizin-7a(5H)-yl)methoxy)-4-(2-(2-hydroxyethyl)-7,8-dihydro-4H-pyrazolo[1,5-a][1,4]diazepin-5(6H)-yl)-5,8-dihydropyrido[3,4-d]pyrimidin-7(6H)-yl)naphthalen-2-ol

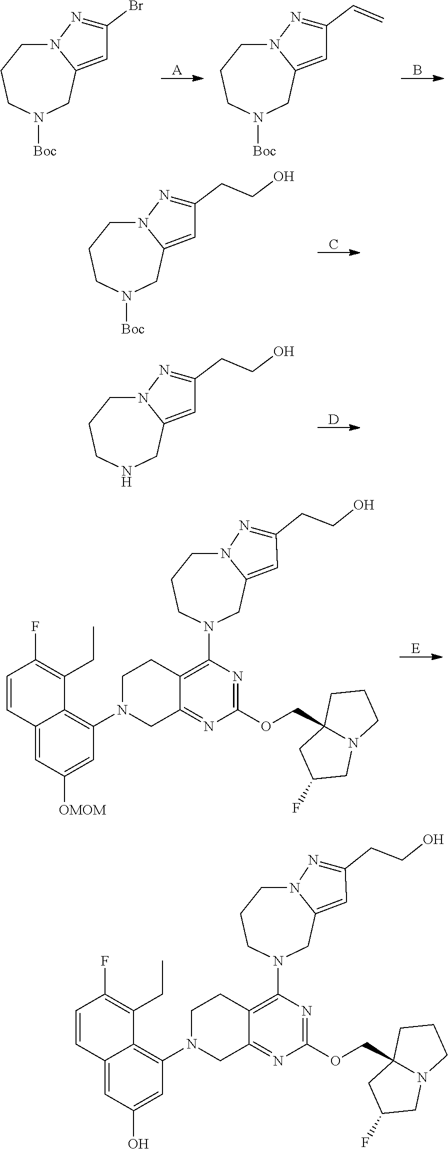

Step A. tert-butyl 2-vinyl-7,8-dihydro-4H-pyrazolo[1,5-a][1,4]diazepine-5(6H)-carboxylate: To a mixture of tert-butyl 2-bromo-7,8-dihydro-4H-pyrazolo[1,5-a][1,4]diazepine-5(6H)-carboxylate (1.00 g, 1.0 equiv), 4,4,5,5-tetramethyl-2-vinyl-1,3,2-dioxaborolane (977 mg, 2.0 equiv), cataCXium A Pd G3 (462 mg, 0.2 equiv) in dioxane (20 mL) and H₂O (4 mL) was added NaHCO₃ (799 mg, 3.0 equiv). The mixture was degassed under vacuum and purged with N₂ several times. The reaction was stirred at 80° C. for 3 hours. The mixture was diluted with water (12 mL) and extracted with ethyl acetate (20 mL). The organic layer was dried over sodium sulfate, concentrated to afford the title compound (260 mg, 28% yield) as yellow oil; LCMS (ESI, M+1). m/z=264.3.

Step B. tert-butyl 2-(2-hydroxyethyl)-7,8-dihydro-4H-pyrazolo[1,5-a][1,4]diazepine-5(6H)-carboxylate: To a solution of tert-butyl 2-vinyl-7,8-dihydro-4H-pyrazolo[1,5-a][1,4]diazepine-5(6H)-carboxylate (260 mg, 1.0 equiv) in THF (20 mL) was added dropwise NaOH (3 M in water, 1.32 mL, 4.0 equiv) at 0° C. The mixture was stirred at 0° C. for 2 hours. BH3-Me2S (10 M, 296 μL, 3.0 equiv) and H₂O₂ (1.38 g, 30% purity, 12 equiv) was added dropwise at 0° C. The reaction was stirred at 25° C. for 14 hours. The mixture was quenched with saturated Na₂SO₃ solution (50 mL) at 0° C., diluted with water (50 mL) and ethyl acetate (80 mL). The organic layer was dried over sodium sulfate, concentrated and purified by reversed-phase HPLC (C18, 0.1% formic acid condition) to afford the title compound (76.0 mg, 27% yield) as yellow oil; LCMS (ESI, M+1): m/z=282.2.

Step C. 2-(5,6,7,8-tetrahydro-4H-pyrazolo[1,5-a][1,4]diazepin-2-yl)ethanol: To a solution of tert-butyl 2-(2-hydroxyethyl)-7,8-dihydro-4H-pyrazolo[1,5-a][1,4]diazepine-5(6H)-carboxylate (76.0 mg, 1.0 equiv) in MeCN (2 mL) was added HCl/dioxane (4 M, 3.17 mL, 47 equiv) at 25° C. The reaction was stirred at 25° C. for 0.5 hours. The mixture was concentrated to afford the title compound (170 mg, crude, HCl salt) as yellow solid.

The last two steps were according to Example 248. The title compound was obtained as a brown solid (FA salt). ¹H NMR (400 MHz, methanol-d₄) δ=7.52 (dd, J=6.0, 8.8 Hz, 1H), 7.15 (t, J=9.2 Hz, 1H), 6.97 (s, 2H), 6.12 (s, 1H), 5.48-5.27 (m, 1H), 5.02-4.95 (m, 1H), 4.81-4.73 (m, 1H), 4.47-4.36 (m, 2H), 4.31-4.15 (m, 3H), 4.07 (br d, J=17.6 Hz, 1H), 4.00-3.90 (m, 1H), 3.80-3.73 (m, 2H), 3.69 (br d, J=17.4 Hz, 1H), 3.58-3.46 (m, 3H), 3.45-3.38 (m, 2H), 3.25-3.13 (m, 3H), 2.82-2.68 (m, 3H), 2.49-1.93 (m, 9H), 1.16-1.06 (m, 3H); LCMS (ESI, M+1): m/z=660.3.

### Example 631

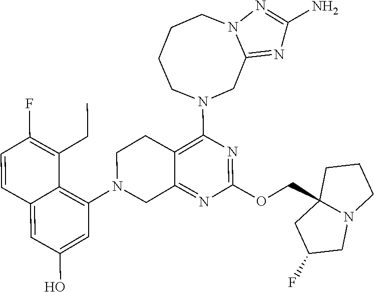 [SMILES: CCc1c(F)ccc2cc(O)cc(N3CCc4c(nc(OC[C@@]56CCCN5C[C@H](F)C6)nc4N4CCCCn5nc(N)nc5C4)C3)c12]

### 4-(4-(2-amino-6,7-dihydro-5H-[1,2,4]triazolo[1,5-a][1,4]diazepin-8(9H)-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-ol

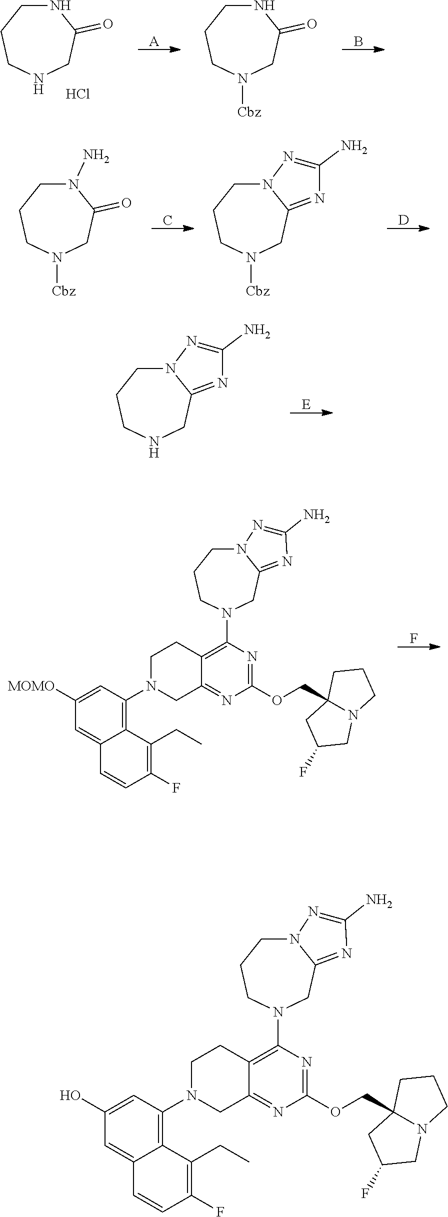

Step A. benzyl 3-oxo-1,4-diazepane-1-carboxylate: To a mixture of 1,4-diazepan-2-one (4.50 g, 1.0 equiv, HCl) and benzyl carbonochloridate (7.65 g, 1.5 equiv) in DCM (45 mL) was added DIEA (38.6 g, 10 equiv). The reaction was stirred at 20° C. for 1 hour. The mixture was diluted with water (80 mL) and extracted with ethyl acetate (30 mL×3). The combined organic layers were washed with brine (2×20 mL), dried over anhydrous sodium sulfate, concentrated, and purified with reversed-phase HPLC [water (FA, 0.1%)/acetonitrile] to afford the title compound (6.30 g, 84% yield) as off-white solid; LCMS (ESI, M+1): m/z=249.0.

Step B. benzyl 4-amino-3-oxo-1,4-diazepane-1-carboxylate: To a mixture of benzyl 3-oxo-1,4-diazepane-1-carboxylate (700 mg, 1.0 equiv) and amino hydrogen sulfate (638 mg, 2.0 equiv) in DMF (7 mL) was added t-BuOK (1.0 M, 7.1 mL, 2.5 equiv). The reaction was stirred at 20° C. for 3 hours. The mixture concentrated and purified by reversed phase flash [water (FA, 0.1%)/acetonitrile] and prep-HPLC (column: Phenomenex luna C18 150*25 mm*10 μm; mobile phase: [water (FA)-ACN]; B %: 8%-38%, 10 min) to afford the title compound (380 mg, 8.0% yield) as yellow oil; LCMS (ESI, M+1): m/z=263.9.

Step C. benzyl 2-amino-6,7-dihydro-5H-[1,2,4]triazolo[1,5-a][1,4]diazepine-8(9H)-carboxylate: To a mixture of benzyl 4-amino-3-oxo-1,4-diazepane-1-carboxylate (320 mg, 1.0 equiv) and cyanamide (307 mg, 6.0 equiv) in EtOH (3.5 mL) was TsOH•H₂O (347 mg, 1.5 equiv). The reaction was stirred at 80° C. for 12 hours. TEA (246 mg, 2.0 equiv) was added into above mixture. The reaction was stirred at 80° C. for 0.5 hours. The mixture was filtered and purified by reversed-phase HPLC [water (FA, 0.1%)/acetonitrile] to afford the title compound (150 mg, 42% yield) as yellow solid;

Step D. 6,7,8,9-tetrahydro-5H-[1,2,4]triazolo[1,5-a][1,4]diazepin-2-amine: To a mixture of benzyl 2-amino-5,6,7,9-tetrahydro-[1,2,4]triazolo[1,5-a][1,4]diazepine-8-carboxylate (120 mg, 1.0 equiv) in MeOH (3.6 mL) and NH₃/MeOH (0.9 mL, 25% purity) was added Pd/C (120 mg, 10% purity). The reaction was degassed and purged with H₂ 3 times. The reaction was stirred at 20° C. for 0.5 hour. The mixture was filtered and concentrated to afford the title compound (63.0 mg, 94% yield) as white solid; LCMS (ESI, M+1): m/z=154.2

The last two steps were according to Example 248. The title compound was obtained as orange solid (FA salt). ¹H NMR (400 MHz, METHANOL-d₄) δ=7.51 (dd, J=6.0, 8.8 Hz, 1H), 7.14 (t, J=9.6 Hz, 1H), 6.98 (br d, J=3.6 Hz, 2H), 5.48-5.27 (m, 1H), 4.67 (br d, J=16.4 Hz, 2H), 4.37-4.16 (m, 2H), 4.15-3.91 (m, 5H), 3.74-3.63 (m, 1H), 3.56-3.36 (m, 6H), 3.27-3.08 (m, 3H), 2.77-2.66 (m, 1H), 2.50-2.15 (m, 5H), 2.12-1.91 (m, 3H), 1.11 (br t, J=7.2 Hz, 3H); LCMS (ESI, M+1): m/z=632.5.

### Example 632

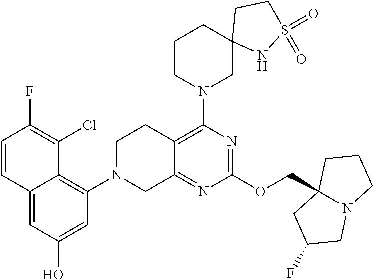 [SMILES: O=S1(=O)CCC2(CCCN(c3nc(OC[C@@]45CCCN4C[C@H](F)C5)nc4c3CCN(c3cc(O)cc5ccc(F)c(Cl)c35)C4)C2)N1]

### 7-(7-(8-chloro-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-2-thia-1,7-diazaspiro[4.5]decane 2,2-dioxide

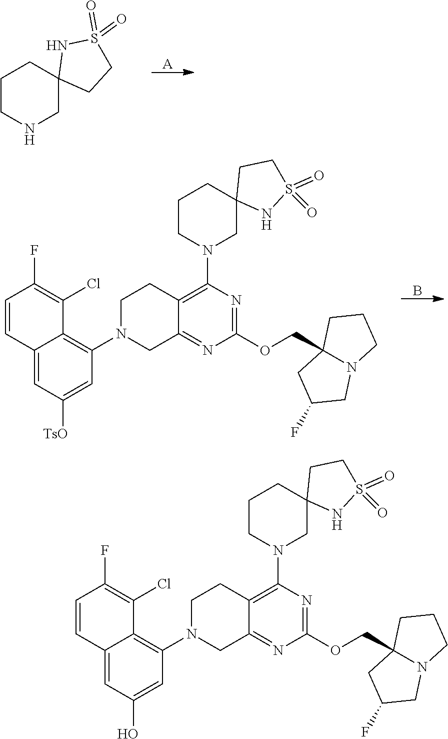

Step A. 5-chloro-4-(4-(2,2-dioxido-2-thia-1,7-diazaspiro[4.5]decan-7-yl)-2-(((2R,7aS)-2-fluorohexahydro-1H-pyrrolizin-7a-yl)methoxy)-5,6-dihydropyrido[3,4-d] pyrimidin-7(8H)-yl)-6-fluoronaphthalen-2-yl 4-methylbenzenesulfonate: To a solution of 5-chloro-6-fluoro-4-(2-(((2R,7aS)-2-fluorotetrahydro-1H-pyrrolizin-7a(5H)-yl)methoxy)-4-(tosyloxy)-5,8-dihydropyrido[3,4-d]pyrimidin-7(6H)-yl)naphthalen-2-yl 4-methylbenzenesulfonate (80.0 mg, 1.0 equiv) and 2-thia-1,7-diazaspiro[4.5]decane 2,2-dioxide (22.5 mg, 1.2 equiv) in DMF (0.50 mL) were added DIEA (63.7 mg,, 5.0 equiv) and 4 Å molecular sieve (30 mg). The reaction was stirred at 40° C. for 12 hours. The mixture was filtered, concentrated and purified with prep-HPLC [column: Phenomenex luna C18 150×25 mm×10 μm; A: [water (FA)]; B:ACN; B %: 26%-56%, over 10 minutes] to afford the title compound (50.0 mg, 60% yield) as a white solid; LCMS (ESI, M+1): m/z=829.2.

Step B. 7-(7-(8-chloro-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-2-thia-1,7-diazaspiro[4.5]decane 2,2-dioxide: To a solution of 5-chloro-4-(4-(2,2-dioxido-2-thia-1,7-diazaspiro[4.5]decan-7-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-6-fluoronaphthalen-2-yl 4-methylbenzenesulfonate (40.0 mg, 1.0 equiv) in THF (1.00 mL) and H₂O (1.00 mL) was added NaOH (11.6 mg, 6.0 equiv). The reaction was stirred at 60° C. for 36 hours. The mixture was neutralized with 2 M HCl (1.00 mL), concentrated and purified with prep-HPLC [column: Phenomenex luna C18 150×25 mm×10 μm; A: water(FA), B: ACN, B %: 18%-48% over 10 min] to afford title compound (10.1 mg, FA salt) as yellow solid. ¹H NMR (400 MHz, METHANOL-d₄) δ=8.53 (s, 1H), 7.67-7.54 (m, 1H), 7.32-7.22 (m, 1H), 6.99-6.89 (m, 2H), 5.46-5.22 (m, 1H), 4.33-4.27 (m, 1H), 4.24 (br s, 1H), 4.23-4.15 (m, 1H), 4.03-3.80 (m, 1H), 3.76-3.34 (m, 7H), 3.27-3.17 (m, 2H), 3.16-3.03 (m, 2H), 2.71-2.58 (m, 1H), 2.50-2.35 (m, 1H), 2.34-2.12 (m, 4H), 2.11-1.65 (m, 8H); LCMS (ESI, M+1): m/z=675.3

### Example 633

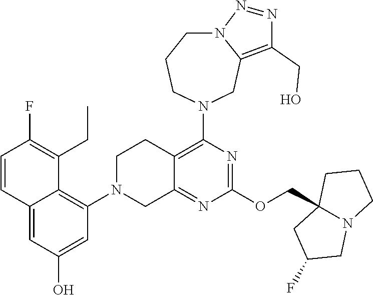 [SMILES: CCc1c(F)ccc2cc(O)cc(N3CCc4c(nc(OC[C@@]56CCCN5C[C@H](F)C6)nc4N4CCCn5nnc(CO)c5C4)C3)c12]

### 5-ethyl-6-fluoro-4-(2-(((2R,7aS)-2-fluorotetrahydro-1H-pyrrolizin-7a(5H)-yl)methoxy)-4-(3-(hydroxymethyl)-7,8-dihydro-4H-pyrazolo[1,5-a][1,4]diazepin-5(6H)-yl)-5,8-dihydropyrido[3,4-d]pyrimidin-7(6H)-yl)naphthalen-2-ol

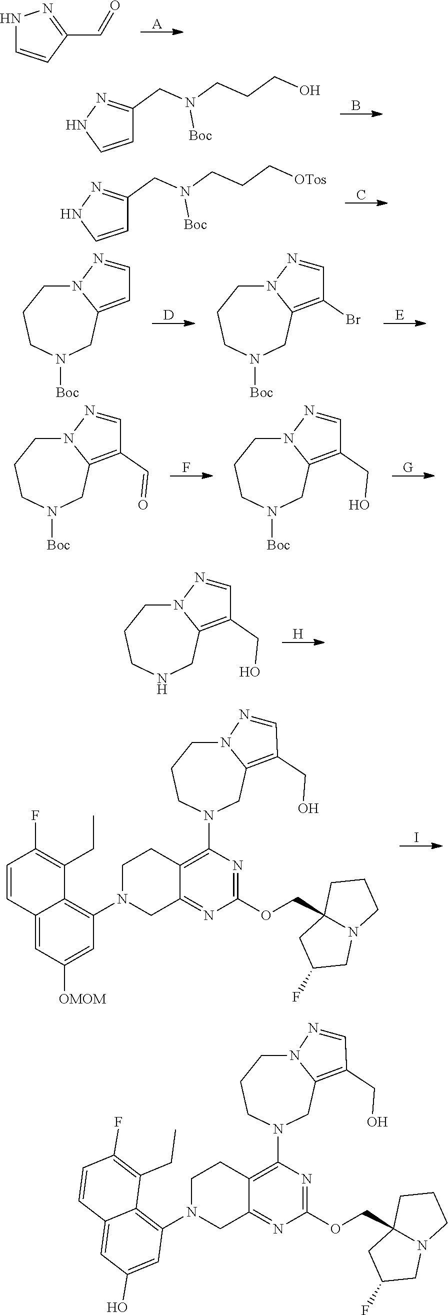

Step A. tert-butyl ((1H-pyrazol-3-yl)methyl)(3-hydroxypropyl)carbamate: To a mixture of 1H-pyrazole-3-carbaldehyde (30.0 g, 1.0 equiv) and 3-aminopropan-1-ol (35.2 g, 1.5 equiv) in EtOH (300 mL) was added NaBH4 (26.0 g, 2.2 equiv). The reaction was stirred at 0° C. for 2 hours. The mixture was quenched with H₂O (300 mL) at 0° C. (Boc)20 (112 g, 2.0 equiv) was added into above mixture. The reaction was stirred at 20° C. for 12 hours. The mixture was concentrated to remove EtOH. The residue was diluted with H₂O (300 mL) and extracted with ethyl acetate (3×300 mL). The combined organic layers were dried over anhydrous sodium sulfate, concentrated purified with column chromatography (SiO₂, Petroleum ether/Ethyl acetate=10/1 to 0/1) and reversed-phase HPLC (0.1% FA condition) to afford the title compound (40.2 g, 30% yield) as yellow solid; LCMS (ESI, M-99): m/z=156.4.

Step B. 3-(((1H-pyrazol-3-yl)methyl)(tert-butoxycarbonyl)amino)propyl 4-methylbenzenesulfonate: To a solution of tert-butyl ((1H-pyrazol-3-yl)methyl)(3-hydroxypropyl)carbamate (11.0 g, 1.0 equiv), DIEA (16.7 g, 3.0 equiv) and DMAP (263 mg, 0.05 equiv) in DCM (100 mL) was added 4-methylbenzene-1-sulfonyl chloride (16.4 g, 2.0 equiv). The reaction was stirred at 20° C. for 0.5 hours. The mixture was concentrated and purified with reversed-phase HPLC (0.1% FA condition) to afford the title compound (5.00 g, 28% yield) as yellow solid; LCMS (ESI, M-99): m/z=310.3.

Step C. tert-butyl 7,8-dihydro-4H-pyrazolo[1,5-a][1,4]diazepine-5(6H)-carboxylate: To a solution of 3-(((1H-pyrazol-3-yl)methyl)(tert-butoxycarbonyl)amino)propyl 4-methylbenzenesulfonate (8.00 g, 1.0 equiv) in DMAC (80 mL) was added NaH (2.34 g, 60% purity, 3.0 equiv). The reaction was stirred at 0-20° C. for 2 hours. The mixture was quenched with H₂O (100 mL) at 0° C. and extracted with ethyl acetate (3×100 mL). The combined organic layers were dried over anhydrous sodium sulfate, concentrated and purified by reversed-phase HPLC (0.1% FA condition) to afford the title compound (7.10 g, 73% yield) as yellow oil.

Step D. tert-butyl 3-bromo-7,8-dihydro-4H-pyrazolo[1,5-a][1,4]diazepine-5(6H)-carboxylate: To a solution of tert-butyl 7,8-dihydro-4H-pyrazolo[1,5-a][1,4]diazepine-5(6H)-carboxylate (7.00 g, 1.0 equiv) in ACN (70 mL) was added NBS (7.88 g, 1.5 equiv). The reaction was stirred at 80° C. for 1 hour. The mixture was concentrated and purified by reversed-phase HPLC (0.1% FA condition) to afford the title compound (6.20 g, 65% yield) as white solid. LCMS (ESI, M+1): m/z=316.0.

Step E. tert-butyl 3-formyl-7,8-dihydro-4H-pyrazolo[1,5-a][1,4]diazepine-5(6H)-carboxylate: To a solution of tert-butyl 3-bromo-7,8-dihydro-4H-pyrazolo[1,5-a][1,4]diazepine-5(6H)-carboxylate (500 mg, 1.0 equiv) in THE (10 mL) was added n-BuLi (1 M, 4.74 mL, 3.0 equiv) at −60° C. The reaction was stirred at −60° C. for 0.5 hours. DMF (578 mg, 5.0 equiv) was added into above mixture. The reaction was stirred at −60° C. for 0.5 hours. The mixture was quenched with H₂O (10 mL) at 0° C. and extracted with EtOAc (3×20 mL). The combined organic layers were dried over anhydrous sodium sulfate, concentrated, and purified by reversed-phase HPLC(0.1% FA condition) to afford the title compound (210 mg, 47% yield) as yellow oil. LCMS (ESI, M+1): m/z=266.3.

Step F. tert-butyl 3-(hydroxymethyl)-7,8-dihydro-4H-pyrazolo[1,5-a][1,4]diazepine-5(6H)-carboxylate To a solution of tert-butyl 3-formyl-7,8-dihydro-4H-pyrazolo[1,5-a][1,4]diazepine-5(6H)-carboxylate (100 mg, 1.0 equiv) in EtOH (1 mL) was added NaBH4 (20.0 mg, 1.5 equiv). The reaction was stirred at 0° C. for 1 hour. The mixture was quenched with saturated NH₄Cl solution (3.0 mL) at 0° C. and extracted with EtOAc (3×2 mL). The combined organic layers were dried over anhydrous sodium sulfate and concentrated to afford the title compound (110 mg, 98% yield) as yellow solid. LCMS (ESI, M+1): m/z=268.1

Step G. (5,6,7,8-tetrahydro-4H-pyrazolo[1,5-a][1,4]diazepin-3-yl)methanol: A mixture of tert-butyl 3-(hydroxymethyl)-7,8-dihydro-4H-pyrazolo[1,5-a][1,4]diazepine-5(6H)-carboxylate (80.0 mg, 1.0 equiv) in HCl•MeOH (4 M, 0.8 mL, 11 equiv) was stirred at 20° C. for 0.5 hours. The mixture was concentrated to afford the title compound (55.0 mg, crude) as yellow oil.

The last two steps were according to Example 248. The title compound was obtained as white solid (FA salt). ¹H NMR (400 MHz, METHANOL-d₄) δ=7.51 (dd, J=6.0, 9.2 Hz, 1H), 7.36 (s, 1H), 7.14 (t, J=9.2 Hz, 1H), 6.96 (s, 2H), 5.41-5.21 (m, 1H), 5.05-4.98 (m, 1H), 4.79-4.76 (m, 1H), 4.62-4.58 (m, 2H), 4.50-4.36 (m, 2H), 4.20-3.95 (m, 6H), 3.65 (br d, J=18.0 Hz, 1H), 3.53-3.46 (m, 1H), 3.44-3.36 (m, 3H), 3.24-3.12 (m, 2H), 3.07 (dt, J=5.6, 9.6 Hz, 1H), 2.70 (br d, J=14.4 Hz, 1H), 2.34-2.19 (m, 3H), 2.17-1.95 (m, 5H), 1.95-1.82 (m, 1H), 1.11 (t, J=7.2 Hz, 3H). LCMS (ESI, M+1): m/z=646.6.

### Example 634

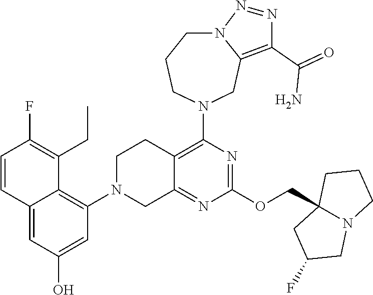 [SMILES: CCc1c(F)ccc2cc(O)cc(N3CCc4c(nc(OC[C@@]56CCCN5C[C@H](F)C6)nc4N4CCCn5nnc(C(N)=O)c5C4)C3)c12]

### 5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepine-3-carboxamide

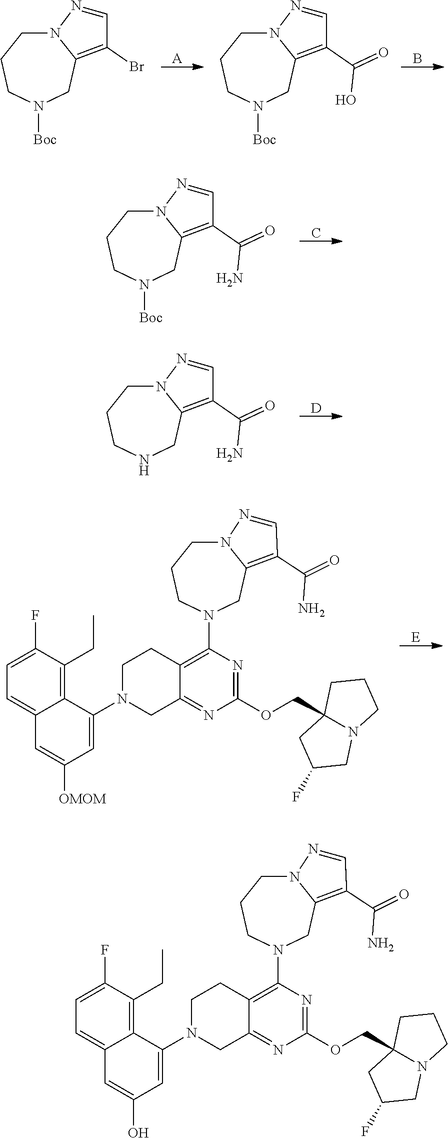

Step A. 5-(tert-butoxycarbonyl)-5,6,7,8-tetrahydro-4H-pyrazolo[1,5-a][1,4]diazepine-3-carboxylic acid: To a solution of tert-butyl 3-bromo-7,8-dihydro-4H-pyrazolo[1,5-a][1,4]diazepine-5(6H)-carboxylate (300 mg, 1.0 equiv) in THF (5 mL) was added n-BuLi (570 uL, 1.5 equiv) at −70° C. The reaction was stirred at −70° C. for 5 mins. Dry ice (417 mg, 10 equiv) was added into above mixture. The reaction was stirred at −70° C. for 5 mins. The mixture was quenched with water (10 mL). The mixture was adjusted to pH=4 with HCl (2 mL, 2 M) and extracted with ethyl acetate (3×10 mL). The combined organic layers were dried over anhydrous sodium sulfate and concentrated to afford the title compound (230 mg, 43% yield) as white solid; LCMS (ESI, M+1): m/z=282.0.

Step B. tert-butyl 3-carbamoyl-7,8-dihydro-4H-pyrazolo[1,5-a][1,4]diazepine-5(6H)-carboxylate: To a mixture of 5-(tert-butoxycarbonyl)-5,6,7,8-tetrahydro-4H-pyrazolo[1,5-a][1,4]diazepine-3-carboxylic acid (180 mg, 1.0 equiv) and NH₄Cl (137 mg, 4.0 equiv) in DMF (2 mL) were added HATU (730 mg, 3.0 equiv) and DIEA (827 mg, 10 equiv). The reaction was stirred at 25° C. for 1 hour. The mixture was filtered and purified with reversed phase flash [C18, water (FA, 0.1%)/acetonitrile] to afford title compound (130 mg, 72% yield) as yellow oil. LCMS (ESI, M+1): m/z=281.4.

Step C. 5,6,7,8-tetrahydro-4H-pyrazolo[1,5-a][1,4]diazepine-3-carboxamide: To a mixture of tert-butyl 3-carbamoyl-7,8-dihydro-4H-pyrazolo[1,5-a][1,4]diazepine-5(6H)-carboxylate (120 mg, 1.0 equiv) in MeCN (0.5 mL) was added HCl•dioxane (1.50 mL, 14 equiv). The reaction was stirred at 25° C. for 0.5 hour. The mixture was concentrated to afford title compound (150 mg, crude, HCl) as yellow oil.

The last two steps were according to Example 248. The title compound was obtained as yellow solid. ¹H NMR (400 MHz, METHANOL-d₄) δ=7.83 (d, J=3.6 Hz, 1H), 7.50 (dd, J=5.6, 9.2 Hz, 1H), 7.14 (t, J=9.2 Hz, 1H), 7.03-6.88 (m, 2H), 5.36-5.28 (m, 1H), 5.19-5.06 (m, 2H), 4.49 (br dd, J=8.0, 14.4 Hz, 1H), 4.40-4.28 (m, 1H), 4.11-3.84 (m, 5H), 3.63 (br d, J=17.2 Hz, 1H), 3.51-3.35 (m, 4H), 3.25-3.08 (m, 4H), 3.01-2.92 (m, 1H), 2.64 (br d, J=14.8 Hz, 1H), 2.45-2.32 (m, 1H), 2.30-2.01 (m, 4H), 1.99-1.79 (m, 3H), 1.11 (t, J=7.2 Hz, 3H); LCMS (ESI, M+1): m/z=659.5.

### Example 635

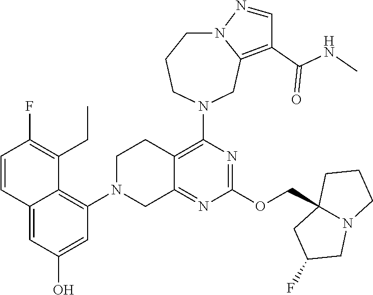 [SMILES: CCc1c(F)ccc2cc(O)cc(N3CCc4c(nc(OC[C@@]56CCCN5C[C@H](F)C6)nc4N4CCCn5ncc(C(=O)NC)c5C4)C3)c12]

### 5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-N-methyl-5,6,7,8-tetrahydro-4H-pyrazolo[1,5-a][1,4]diazepine-3-carboxamide

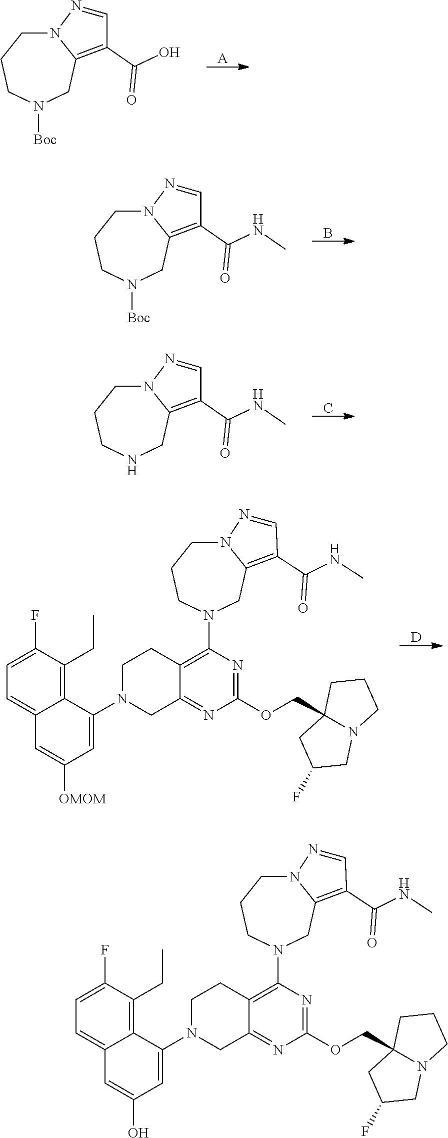

Step A. tert-butyl 3-(methylcarbamoyl)-7,8-dihydro-4H-pyrazolo[1,5-a][1,4]diazepine-5(6H)-carboxylate: To a mixture of 5-(tert-butoxycarbonyl)-5,6,7,8-tetrahydro-4H-pyrazolo[1,5-a][1,4]diazepine-3-carboxylic acid (190 mg, 1.0 equiv) and methanamine hydrochloride (68.4 mg, 1.5 eq, HCl) in DMF (2.0 mL) were added HATU (771 mg, 3.0 equiv) and DIEA (873 mg, 10 equiv). The reaction was stirred at 25° C. for 1 hour. The mixture was filtered and purified by reversed-phase HPLC[water (FA, 0.1%)/acetonitrile] to afford the title compound (150 mg, 74% yield) as yellow oil; LCMS (ESI, M-55): m/z=239.2.

Step B. N-methyl-5,6,7,8-tetrahydro-4H-pyrazolo[1,5-a][1,4]diazepine-3-carboxamide: To a mixture of tert-butyl 3-(methylcarbamoyl)-7,8-dihydro-4H-pyrazolo[1,5-a][1,4]diazepine-5(6H)-carboxylate (140 mg, 1.0 equiv) in ACN (1.5 mL) was added HCl•dioxane (4 M, 1.40 mL, 12 equiv). The reaction was stirred at 25° C. for 0.5 hour. The mixture was concentrated to afford the title compound (140 mg, crude) as green solid.

Step C. 5-(7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d] pyrimidin-4-yl)-N-methyl-5,6,7,8-tetrahydro-4H-pyrazolo[1,5-a][1,4]diazepine-3-carboxamide: To a mixture of 7-(8-ethyl-7-fluoro-3-(methoxymethoxy)naphthalen-1-yl)-2-(((2R,7aS)-2-fluorohexahydro-1H-pyrrolizin-7a-yl)methoxy)-5,6,7,8-tetrahydropyrido[3,4-d]pyrimidin-4-yl 4-methylbenzenesulfonate (200 mg, 1.0 equiv) and N-methyl-5,6,7,8-tetrahydro-4H-pyrazolo[1,5-a][1,4]diazepine-3-carboxamide (133 mg, HCl) in DMF (2.0 mL) were added DIEA (744 mg, 20 equiv) and 4 Å molecular sieve (5.00 mg). The reaction was stirred at 60° C. for 24 hours. The mixture was filtered and purified by reversed-phase HPLC[water (FA, 0.1%)/acetonitrile] to afford the title compound (150 mg, 66% yield) as yellow oil; LCMS (ESI, M+1): m/z=717.6

The last two steps were according to Example 248. The title compound was obtained as pink solid. ¹H NMR (400 MHz, METHANOL-d₄) δ=7.76 (d, J=1.6 Hz, 1H), 7.50 (dd, J=5.6, 8.8 Hz, 1H), 7.13 (t, J=9.2 Hz, 1H), 6.98-6.94 (m, 2H), 5.33-5.10 (m, 3H), 4.49 (br dd, J=7.2, 14.0 Hz, 1H), 4.38-4.28 (m, 1H), 4.10-3.88 (m, 5H), 3.64 (br d, J=17.6 Hz, 1H), 3.35 (br s, 4H), 3.24-3.07 (m, 4H), 2.96 (dt, J=5.6, 9.2 Hz, 1H), 2.83 (s, 3H), 2.59 (br d, J=15.2 Hz, 1H), 2.44-2.32 (m, 1H), 2.29-2.07 (m, 3H), 2.06-1.80 (m, 4H), 1.10 (t, J=7.2 Hz, 3H); LCMS (ESI, M+1): m/z=673.1.

### Example 636

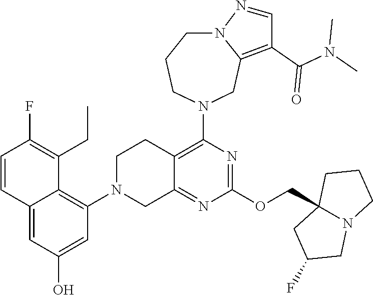 [SMILES: CCc1c(F)ccc2cc(O)cc(N3CCc4c(nc(OC[C@@]56CCCN5C[C@H](F)C6)nc4N4CCCn5ncc(C(=O)N(C)C)c5C4)C3)c12]

### 5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-3-carboxamide

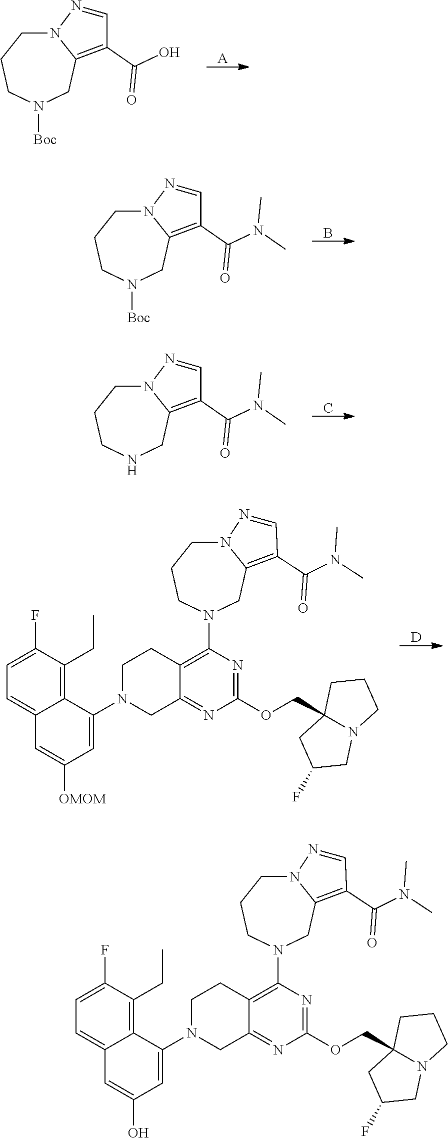

Step A. tert-butyl 3-(dimethylcarbamoyl)-7,8-dihydro-4H-pyrazolo[1,5-a][1,4]diazepine-5(6H)-carboxylate: To a mixture of 5-(tert-butoxycarbonyl)-5,6,7,8-tetrahydro-4H-pyrazolo[1,5-a][1,4]diazepine-3-carboxylic acid (200 mg, 1.0 equiv) and dimethylamine (2 M, 711 μL, 2.0 equiv) in DMF (2.0 mL) were added HATU (811 mg, 3.0 equiv) and DIEA (919 mg, 10 equiv). The reaction was stirred at 25° C. for 1 hour. The mixture was filtered and purified by reversed phase flash [water (FA, 0.1%)/acetonitrile] to afford the title compound (175 mg, 79% yield) as white oil; LCMS (ESI, M+1): m/z=309.1.

Step B. N,N-dimethyl-5,6,7,8-tetrahydro-4H-pyrazolo[1,5-a][1,4]diazepine-3-carboxamide: To a mixture of tert-butyl 3-(dimethylcarbamoyl)-7,8-dihydro-4H-pyrazolo[1,5-a][1,4]diazepine-5(6H)-carboxylate (170 mg, 1.0 equiv) in ACN (1.0 mL) was added HCl•dioxane (4 M, 2.0 mL, 14 equiv). The reaction was stirred at 25° C. for 0.5 hour. The mixture was concentrated to afford the title compound (210 mg, crude, HCl) as yellow oil.

The last two steps were according to Example 248. The title compound was obtained as pink solid. ¹H NMR (400 MHz, METHANOL-d₄) δ=7.63 (s, 1H), 7.52 (dd, J=5.6, 9.2 Hz, 1H), 7.15 (t, J=9.2 Hz, 1H), 6.96 (d, J=2.8 Hz, 2H), 5.39-5.17 (m, 1H), 4.95 (br d, J=2.4 Hz, 2H), 4.62-4.49 (m, 2H), 4.47-4.37 (m, 1H), 4.16-4.10 (m, 1H), 4.07 (s, 2H), 3.96-3.84 (m, 1H), 3.69 (br d, J=17.6 Hz, 1H), 3.45-3.34 (m, 3H), 3.27-3.18 (m, 3H), 3.17-3.06 (m, 5H), 3.05-2.87 (m, 4H), 2.56-2.45 (m, 1H), 2.27 (br d, J=4.4 Hz, 2H), 2.24-2.05 (m, 3H), 2.01-1.81 (m, 3H), 1.12 (t, J=7.2 Hz, 3H); LCMS (ESI, M+1): m/z=687.4.

### Example 637

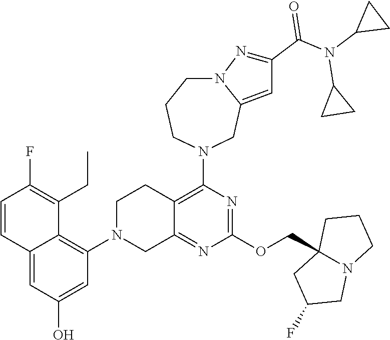 [SMILES: CCc1c(F)ccc2cc(O)cc(N3CCc4c(nc(OC[C@@]56CCCN5C[C@H](F)C6)nc4N4CCCn5nc(C(=O)N(C6CC6)C6CC6)cc5C4)C3)c12]

### N,N-dicyclopropyl-5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-5,6,7,8-tetrahydro-4H-pyrazolo[1,5-a][1,4]diazepine-2-carboxamide

Synthesized according to Example 233 except that HCl instead of TFA was used in the last step. The title compound was obtained as light yellow solid; ¹H NMR (400 MHz, METHANOL-d₄) δ=7.51 (dd, J=5.6, 8.8 Hz, 1H), 7.15 (t, J=9.2 Hz, 1H), 6.96 (d, J=1.2 Hz, 2H), 6.55 (d, J=0.8 Hz, 1H), 5.36-5.15 (m, 1H), 5.09-4.99 (m, 1H), 4.83-4.76 (m, 1H), 4.58-4.49 (m, 2H), 4.29-4.17 (m, 1H), 4.13-3.89 (m, 4H), 3.67 (dd, J=4.8, 17.6 Hz, 1H), 3.57-3.47 (m, 1H), 3.45-3.33 (m, 2H), 3.27-3.12 (m, 5H), 2.98 (dt, J=5.6, 9.2 Hz, 1H), 2.87-2.66 (m, 3H), 2.39-2.11 (m, 3H), 2.10-1.78 (m, 5H), 1.10 (t, J=7.2 Hz, 3H), 0.98-0.52 (m, 8H); LCMS (ESI, M+1): m/z=739.2

### Example 638

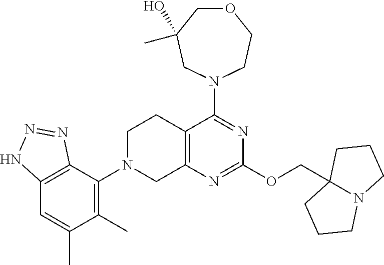 [SMILES: Cc1cc2[nH]nnc2c(N2CCc3c(nc(OCC45CCCN4CCC5)nc3N3CCOC[C@@](C)(O)C3)C2)c1C]

### (S)-4-(7-(5, 6-dimethyl-1H-benzo[d][1,2,3]triazol-4-yl)-2-((tetrahydro-1H-pyrrolizin-7a(5H)-yl)methoxy)-5,6,7,8-tetrahydropyrido[3,4-d]pyrimidin-4-yl)-6-methyl-1,4-oxazepan-6-ol

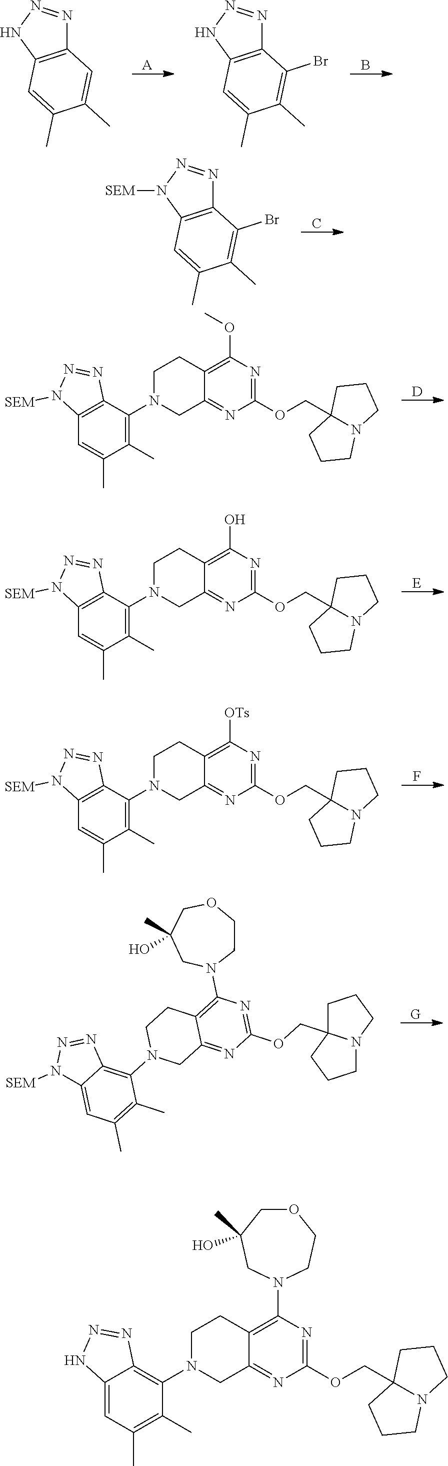

Step A. 4-bromo-5,6-dimethyl-1H-benzo[d][1,2,3]triazole: To a mixture of 5,6-dimethyl-1H-benzo[d][1,2,3]triazole (1.00 g, 1.0 equiv) in AcOH (10 mL) was added a solution of Br2 (1.52 g, 1.4 equiv) in AcOH (10 mL) at 50° C. The mixture was stirred at 50° C. for 1 hour. The mixture was diluted with water (30 mL) and extracted with ethyl acetate (50 mL). The organic layer was dried over sodium sulfate and concentrated and purified by reversed-phase flash (C18, 0.1% FA condition) to afford the title compound (500 mg, 32% yield) as a white solid; LCMS (ESI, M+1, M+3): m/z=225.9, 227.9.

Step B. 4-bromo-5,6-dimethyl-1-((2-(trimethylsilyl)ethoxy)methyl)-1H-benzo[d][1,2,3]triazole: To a solution of 4-bromo-5,6-dimethyl-1H-benzo[d][1,2,3]triazole (300 mg, 1 equiv) in THF (9 mL) was added NaH (106 mg, 60% purity, 2.0 equiv) at 0° C. portionwise. The mixture was stirred at 0° C. for 0.5 hour. Then to the mixture was added SEM-C1 (332 mg, 1.5 equiv) at 0° C. The mixture was stirred at 0° C. for 1 hour and then quenched with water (20 mL) at 0° C. and extracted with ethyl acetate (30 mL). The organic layer was dried over sodium sulfate, concentrated to afford the title compound (245 mg, crude) as a yellow oil.

Step C. 7-(5,6-dimethyl-1-((2-(trimethylsilyl)ethoxy)methyl)-1H-benzo[d]1[1,2,3]triazol-4-yl)-4-methoxy-2-((tetrahydro-1H-pyrrolizin-7a(5H)-yl)methoxy)-5,6,7,8-tetrahydropyrido[3,4-d] pyrimidine: To a solution of 4-methoxy-2-((tetrahydro-1H-pyrrolizin-7a(5H)-yl)methoxy)-5,6,7,8-tetrahydropyrido[3,4-d]pyrimidine (2.3 g, 1.0 equiv) and 4-bromo-5,6-dimethyl-1-((2-(trimethylsilyl)ethoxy)methyl)-1H-benzo[d][1,2,3]triazole (2.96 g, 1.1 equiv) in dioxane (50 mL) were added Cs₂CO₃ (6.15 g, 2.5 equiv) and Xantphos Pd G4 (727 mg, 0.1equiv). The reaction was degassed and purged with nitrogen 3 times. The reaction was stirred at 90° C. for 12 hours. The mixture was diluted with water (250 mL) and extracted with EtOAc (4×80 mL). The combined organic phases were washed with brine (50 mL), dried over anhydrous sodium sulfate, concentrated and purified with reversed phase flash [water (FA, 0.1%)/acetonitrile=3/2] to afford the title compound (4.1 g, 93% yield) as yellow solid; LCMS (ESI, M+1): m/z=580.3.

Step D. 7-(5,6-dimethyl-1-((2-(trimethylsilyl)ethoxy)methyl)-1H-benzo[d][1,2,3]triazol-4-yl)-2-((tetrahydro-1H-pyrrolizin-7a(5H)-yl)methoxy)-5,6,7,8-tetrahydropyrido[3,4-d] pyrimidin-4-ol: To a solution of 7-(5,6-dimethyl-1-((2-(trimethylsilyl)ethoxy)methyl)-1H-benzo[d][1,2,3]triazol-4-yl)-4-methoxy-2-((tetrahydro-1H-pyrrolizin-7a(5H)-yl)methoxy)-5,6,7,8-tetrahydropyrido[3,4-d]pyrimidine (4.0 g, 1.0 equiv) in DMAc (50 mL) was added ethylsulfanylsodium (1.74 g, 3.0 equiv). The reaction was stirred at 60° C. for 2 hours. The mixture was quenched with saturated NH₄Cl aqueous (100 mL) and H₂O (900 mL) at 0° C. The mixture was extracted with EtOAc (6×100 mL). The combined organic layers were washed with brine (3×300 mL), dried over anhydrous sodium sulfate and concentrated to afford the title compound (3.9 g, 99% yield) as yellow solid; LCMS (ESI, M+1): m/z=566.5.

Step E. 7-(5,6-dimethyl-1-((2-(trimethylsilyl)ethoxy)methyll)-1H-benzo[d][1,2,3]triazol-4-yl)-2-((tetrahydro-1H-pyrrolizin-7a(5H)-yl)methoxy)-5,6,7,8-tetrahydropyrido[3,4-d] pyrimidin-4-yl 4-methylbenzenesulfonate: To a solution of 7-(5,6-dimethyl-1-((2-(trimethylsilyl)ethoxy)methyl)-1H-benzo[d][1,2,3]triazol-4-yl)-2-((tetrahydro-1H-pyrrolizin-7a(5H)-yl)methoxy)-5,6,7,8-tetrahydropyrido[3,4-d] pyrimidin-4-ol (3.8 g, 1.0 equiv) and DIPEA (2.60 g, 3.0 equiv) in THF (40 mL) were added DMAP (82.1 mg, 0.1 equiv) and TosCl (1.60 g, 1.3 equiv) at 0° C. The reaction was stirred at 25° C. for 18 hours. The mixture was diluted with H₂O (100 mL) and extracted with EtOAc (4×50 mL). The combined organic layers were washed with brine (20 mL), dried over anhydrous sodium sulfate, concentrated and purified with column chromatography [Al₂O₃, PE/EtOAc=3/1 to 0/1] to afford the title compound (3.35 g, 67% yield) as yellow solid; LCMS (ESI, M+1): m/z=720.3.

Step F. (S)-4-(7-(5,6-dimethl-1-((2-(trimethylsilyl)ethoxy)methyl)-1H-benzo[d][1,2,3]triazol-4-yl)-2-((tetrahydro-1H-pyrrolizin-7a(5H)-yl)methoxy)-5,6,7,8-tetrahydropyrido[3,4-d]pyrimidin-4-yl)-6-methyl-1,4-oxazepan-6-ol: To a solution of 7-(5,6-dimethyl-1-((2-(trimethylsilyl)ethoxy)methyl)-1H-benzo[d][1,2,3]triazol-4-yl)-2-((tetrahydro-1H-pyrrolizin-7a(5H)-yl)methoxy)-5,6,7,8-tetrahydropyrido[3,4-d]pyrimidin-4-yl 4-methylbenzenesulfonate (200 mg, 1.0 equiv) and (S)-6-methyl-1,4-oxazepan-6-ol (72.9 mg, 2.0 equiv) in DMF (1 mL) were added DIPEA (71.8 mg, 2.0 equiv) and 4 Å molecular sieve (20.0 mg). The reaction was stirred at 40° C. for 12 hours. The mixture was filtered and purified with reversed phase flash [water (FA, 0.1%)/acetonitrile=3/2] to afford the title compound (150 mg, 79% yield) as light yellow solid; LCMS (ESI, M+1): m/z=679.5.

Step G. (S)-4-(7-(5,6-dimethyl-1H-benzo[d][1,2,3]triazol-4-yl)-2-((tetrahydro-1H-pyrrolizin-7a(5H)-yl)methoxy)-5,6,7,8-tetrahydropyrido[3,4-d]pyrimidin-4-yl)-6-methyl-1,4-oxazepan-6-ol: To a solution of (S)-4-(7-(5,6-dimethyl-1-((2-(trimethylsilyl)ethoxy)methyl)-1H-benzo[d][1,2,3]triazol-4-yl)-2-((tetrahydro-1H-pyrrolizin-7a(5H)-yl)methoxy)-5,6,7,8-tetrahydropyrido[3,4-d]pyrimidin-4-yl)-6-methyl-1,4-oxazepan-6-ol (140 mg, 1.0 equiv) in DCM (1 mL) was added TFA (2.0 mL) dropwise at 0° C. The reaction was stirred at 20° C. for 2 hours. The mixture was concentrated. The residue was dissolved in DCM (0.5 mL) and diethylamine (1.2 mL). The reaction was stirred at 25° C. for 16 hours. The mixture was diluted with H₂O (5 mL) and extracted with DCM (4×5 mL). The combined organic layers were washed with brine (5 mL), dried over anhydrous Na₂SO₄, concentrated and purified with prep-HPLC [Column: 3_Phenomenex Luna C18 75×30 mm×3 μm; mobile phase: water(FA)-ACN; B %:13%-33% over 8 minutes] to afford the title compound (39.0 mg, 33% yield, 0.37 HCOOH) as yellow solid; ¹H NMR (400 MHz, CD30D) δ=7.39 (s, 1H), 4.49-4.43 (m, 4H), 4.17-4.02 (m, 2H), 3.99-3.81 (m, 2H), 3.69-3.46 (m, 8H), 3.25-3.18 (m, 2H), 3.02-2.80 (m, 2H), 2.45 (s, 3H), 2.43 (s, 3H), 2.31-1.98 (m, 8H), 1.21 (s, 3H); LCMS (ESI, M+1): m/z=549.5.

### Example 639

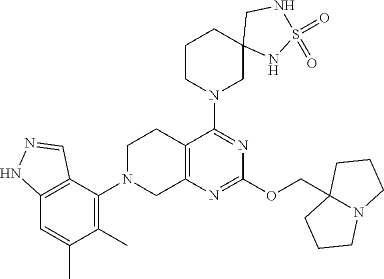 [SMILES: Cc1cc2[nH]ncc2c(N2CCc3c(nc(OCC45CCCN4CCC5)nc3N3CCCC4(CNS(=O)(=O)N4)C3)C2)c1C]

### 7-(7-(5,6-dimethyl-1H-benzo[d][1,2,3]triazol-4-yl)-2-((tetrahydro-1H-pyrrolizin-7a(5H)-yl)methoxy)-5,6,7,8-tetrahydropyrido[3,4-d]pyrimidin-4-yl)-2-thia-1,3,7-triazaspiro[4.5]decane 2,2-dioxide

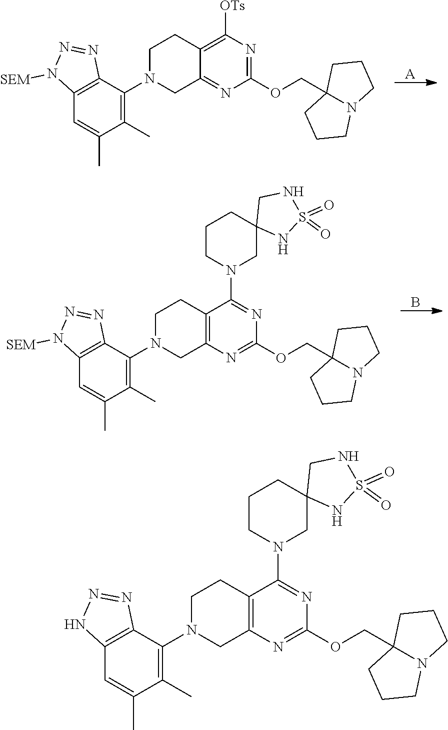

Step A. 7-(7-(5,6-dimethyl-1-((2-(trimethylsilyl)ethoxy)methyl)-1H-benzo[d][1,2,3]triazol-4-yl)-2-((tetrahydro-1H-pyrrolizin-7a(5H)-yl)methoxy)-5,6,7,8-tetrahydropyrido[3,4-d]pyrimidin-4-yl)-2-thia-1,3,7-triazaspiro[4.5]decane 2,2-dioxide: To a solution of 7-(5,6-dimethyl-1-((2-(trimethylsilyl)ethoxy)methyl)-1H-benzo[d][1,2,3]triazol-4-yl)-2-((tetrahydro-1H-pyrrolizin-7a(5H)-yl)methoxy)-5,6,7,8-tetrahydropyrido[3,4-d]pyrimidin-4-yl 4-methylbenzenesulfonate (200 mg, 1.0 equiv) and 2-thia-1,3,7-triazaspiro[4.5]decane 2,2-dioxide (90.0 mg, 1.7 equiv) in DMF (1 mL) were added DIPEA (71.8 mg, 2.0 equiv) and 4 Å molecular sieve (20.0 mg). The reaction was stirred at 40° C. for 12 hours. The mixture was filtered and purified with reversed phase flash [water (FA, 0.1%)/acetonitrile=3/1 to 2/1] to afford the title compound (190 mg, 92% yield) as light yellow solid; LCMS (ESI, M+1): m/z=739.6.

Step B. 7-(7-(5,6-dimethyl-1H-benzo[d][1,2,3]triazol-4-yl)-2-((tetrahydro-1H-pyrrolizin-7a(5H)-yl)methoxy)-5,6,7,8-tetrahydropyrido[3,4-d]pyrimidin-4-yl)-2-thia-1,3,7-triazaspiro[4.5]decane 2,2-dioxide: To a solution of 7-(7-(5,6-dimethyl-1-((2-(trimethylsilyl)ethoxy)methyl)-1H-benzo[d][1,2,3]triazol-4-yl)-2-((tetrahydro-1H-pyrrolizin-7a(5H)-yl)methoxy)-5,6,7,8-tetrahydropyrido[3,4-d]pyrimidin-4-yl)-2-thia-1,3,7-triazaspiro[4.5]decane 2,2-dioxide (190 mg, 1.0 equiv) in THF (1 mL) was added TBAF (1 M in THF, 1.29 mL, 5.0 equiv) dropwise. The reaction was stirred at 50° C. for 14 hours. The mixture was concentrated. The residue was dissolved in DCM (0.5 mL) and diethylamine (1.2 mL). The reaction was stirred at 25° C. for 16 hours. The mixture was diluted with H₂O (20 mL) and extracted with EtOAc (4×10 mL). The combined organic layers were washed with brine (20 mL), dried over anhydrous sodium sulfate, concentrated, and purified with prep-TLC [DCM/MeOH=5/1] and prep-HPLC [Column: Phenomenex C18 75×30 mm×3 μm; mobile phase: water(FA)-ACN; B %:8%-38% over 7 minutes] to afford the title compound (45.9 mg, 27% yield, 0.38 HCOOH) as white solid; ¹H NMR (400 MHz, CD30D) δ=7.40 (s, 1H), 4.58-4.29 (m, 4H), 4.08-4.02 (m, 1H), 3.86-3.80 (m, 1H), 3.68-3.47 (m, 5H), 3.46-3.35 (m, 2H), 3.25-3.15 (m, 3H), 2.98-2.80 (m, 2H), 2.45 (s, 3H), 2.43 (s, 3H), 2.36-2.24 (m, 2H), 2.22-2.08 (m, 4H), 2.07-1.93 (m, 3H), 1.91-1.60 (m, 3H); LCMS (ESI, M+1): m/z=609.3

### Example 640

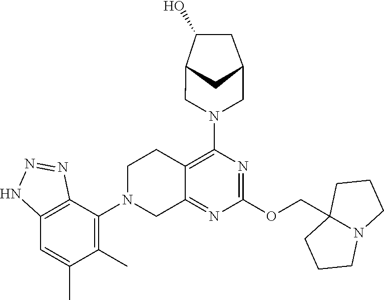 [SMILES: Cc1cc2[nH]nnc2c(N2CCc3c(nc(OCC45CCCN4CCC5)nc3N3C[C@@H]4C[C@H](C3)[C@H](O)C4)C2)c1C]

### (1R,5R,6R)-3-(7-(5,6-dimethyl-1H-benzo[d][1,2,3]triazol-4-yl)-2-((tetrahydro-11H-pyrrolizin-7a(5H)-yl)methoxy)-5,6,7,8-tetrahydropyrido[3,4-d]pyrimidin-4-yl)-3-azabicyclo[3.2.1]octan-6-ol

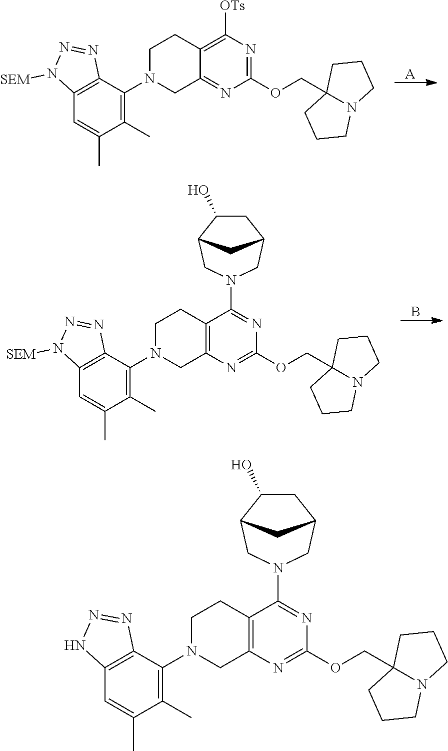

Step A. (1R,5R,6R)-3-(7-(5,6-dimethyl-1-((2-(trimethylsilyl)ethoxy)methyl)-1H-benzo[d][1,2,3]triazol-4-yl)-2-((tetrahydro-1H-pyrrolizin-7a(5H)-yl)methoxy)-5,6,7,8-tetrahydropyrido[3,4-d] pyrimidin-4-yl)-3-azabicyclo[3.2.1]octan-6-ol: To a solution of 7-(5,6-dimethyl-1-((2-(trimethylsilyl)ethoxy)methyl)-1H-benzo[d][1,2,3]triazol-4-yl)-2-((tetrahydro-1H-pyrrolizin-7a(5H)-yl)methoxy)-5,6,7,8-tetrahydropyrido[3,4-d]pyrimidin-4-yl 4-methylbenzenesulfonate (200 mg, 1.0 equiv) and (1R,5R,6R)-3-azabicyclo[3.2.1]octan-6-ol (60.0 mg, 1.7 equiv) in DMF (1 mL) were added DIPEA (71.8 mg, 2.0 equiv) and 4 Å molecular sieve (20.0 mg). The reaction was stirred at 40° C. for 13 hours. The mixture was filtered and purified with reversed phase flash [water (FA, 0.1%)/acetonitrile] to afford the title compound (200 mg, 75% yield) as light yellow solid; LCMS (ESI, M+1): m/z=675.4.

Step B. (1R,5R,6R)-3-(7-(5,6-dimethyl-1H-benzo[d][1,2,3]triazol-4-yl)-2-((tetrahydro-1H-pyrrolizin-7a(5H)-yl)methoxy)-5,6,7,8-tetrahydropyrido[3,4-d]pyrimidin-4-yl)-3-azabicyclo[3.2.1]octan-6-ol: To a solution of (1R,5R,6R)-3-(7-(5,6-dimethyl-1-((2-(trimethylsilyl)ethoxy)methyl)-1H-benzo[d][1,2,3]triazol-4-yl)-2-((tetrahydro-1H-pyrrolizin-7a(5H)-yl)methoxy)-5,6,7,8-tetrahydropyrido[3,4-d]pyrimidin-4-yl)-3-azabicyclo[3.2.1]octan-6-ol (180 mg, 1.0 equiv) in DCM (1.2 mL) was added TFA (2.5 mL) dropwise at 0° C. The reaction was stirred at 20° C. for 2 hours. The mixture was concentrated. The residue was dissolved in DCM (0.5 mL) and diethylamine (1.2 mL). The reaction was stirred at 20° C. for 16 hours. The mixture was diluted with H₂O (10 mL) and extracted with DCM (4×5 mL). The combined organic layers were washed with brine (5 mL), dried over anhydrous sodium sulfate, concentrated, and purified with prep-HPLC [Column: Phenomenex C18 75×30 mm×3 μm; mobile phase: water(FA)-ACN; B %:8%-38% over 7 minutes] to afford the title compound (45.9 mg, 27% yield, 0.72 HCOOH) as yellow solid; ¹H NMR (400 MHz, CD30D) δ=7.39 (s, 1H), 4.48-4.37 (m, 4H), 4.36-4.30 (m, 1H), 4.16-4.11 (m, 1H), 3.66-3.46 (m, 4H), 3.28-3.11 (m, 4H), 3.08-2.83 (m, 2H), 2.44 (s, 3H), 2.42 (s, 3H), 2.30-2.08 (m, 9H), 2.04-2.01 (m, 2H), 1.78-1.70 (m, 2H), 1.42-1.39 (m, 1H); LCMS (ESI, M+1): m/z=545.3.

### Example 641

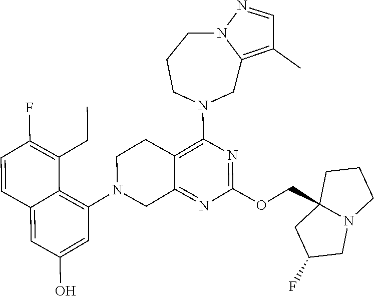 [SMILES: CCc1c(F)ccc2cc(O)cc(N3CCc4c(nc(OC[C@@]56CCCN5C[C@H](F)C6)nc4N4CCCn5ncc(C)c5C4)C3)c12]

### 5-ethyl-6-fluoro-4-(2-(((2R,7aS)-2-fluorotetrahydro-1H-pyrrolizin-7a(5H)-yl)methoxy)-4-(3-methyl-7,8-dihydro-4H-pyrazolo[1,5-a][1,4]diazepin-5(6H)-yl)-5,8-dihydropyrido[3,4-d]pyrimidin-7(6H)-yl)naphthalen-2-ol

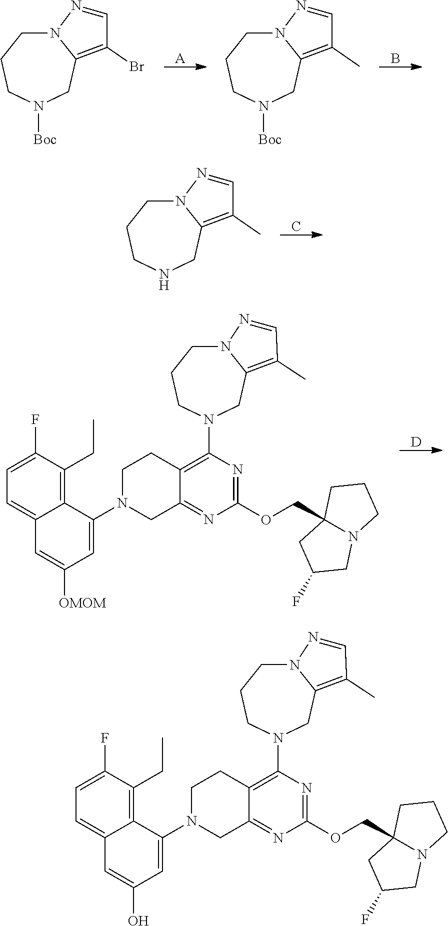

Step A. tert-butyl 3-methyl-7,8-dihydro-4H-pyrazolo[1,5-a][1,4]diazepine-5(6H)-carboxylate: To a solution of tert-butyl 3-bromo-7,8-dihydro-4H-pyrazolo[1,5-a][1,4]diazepine-5(6H)-carboxylate (300 mg, 1.0 equiv) and 2,4,6-trimethyl-1,3,5,2,4,6-trioxatriborinane (714 mg, 795 μL, 50% purity, 3.0 equiv) in DMF (6 mL) were added Pd(dppf)Cl₂ (69.4 mg, 0.10 equiv) and K₂CO₃ (393 mg, 3.0 equiv) at 25° C. The reaction was stirred at 100° C. for 6 hours. The mixture was filtered and purified by reversed phase flash (0.1% FA condition) to afford the title compound (110 mg, 37% yield) as a yellow oil; LCMS (ESI, M+1): m/z=252.3.

Step B. 3-methyl-5,6,7,8-tetrahydro-4H-pyrazolo[1,5-a][1,4]diazepine: To a solution of tert-butyl 3-methyl-7,8-dihydro-4H-pyrazolo[1,5-a][1,4]diazepine-5(6H)-carboxylate (80.0 mg, 1.0 equiv) in methanol (1 mL) was added HCl•MeOH (4 M, 2.00 mL, 25 equiv) at 0° C. The reaction was stirred at 0° C. for 0.5 hours. The mixture was concentrated. The residue was diluted with methanol (3 mL), adjusted to pH=8 with NaHCO₃solid, filtered and concentrated to afford the title compound (40.0 mg, 83% yield) as a colorless oil.

The last two steps were according to Example 248. The title compound was obtained as a yellow solid. ¹H NMR (400 MHz, CHLOROFORM-d) δ=7.44 (br dd, J=3.2, 5.2 Hz, 1H), 7.18 (s, 1H), 7.17-7.09 (m, 1H), 6.95-6.87 (m, 2H), 5.46-5.22 (m, 1H), 4.77 (br d, J=16.4 Hz, 1H), 4.63-4.52 (m, 1H), 4.50-4.34 (m, 2H), 4.33-4.10 (m, 3H), 3.95-3.71 (m, 3H), 3.64-3.20 (m, 6H), 3.14-3.01 (m, 2H), 3.00-2.87 (m, 1H), 2.55-2.39 (m, 2H), 2.35-2.14 (m, 5H), 2.05 (d, J=4.4 Hz, 3H), 2.02-1.98 (m, 2H), 1.09 (t, J=7.2 Hz, 3H); LCMS (ESI, M+1): m/z=630.4.

### Example 642

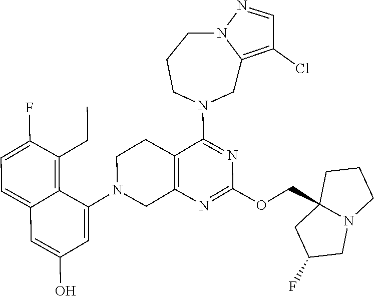 [SMILES: CCc1c(F)ccc2cc(O)cc(N3CCc4c(nc(OC[C@@]56CCCN5C[C@H](F)C6)nc4N4CCCn5ncc(Cl)c5C4)C3)c12]

### 4-(4-(3-chloro-7,8-dihydro-4H-pyrazolo[1,5-a][1,4]diazepin-5(6H)-yl)-2-(((2R,7aS)-2-fluorotetrahydro-1H-pyrrolizin-7a(5H)-yl)methoxy)-5,8-dihydropyrido[3,4-d]pyrimidin-7(6H)-yl)-5-ethyl-6-fluoronaphthalen-2-ol

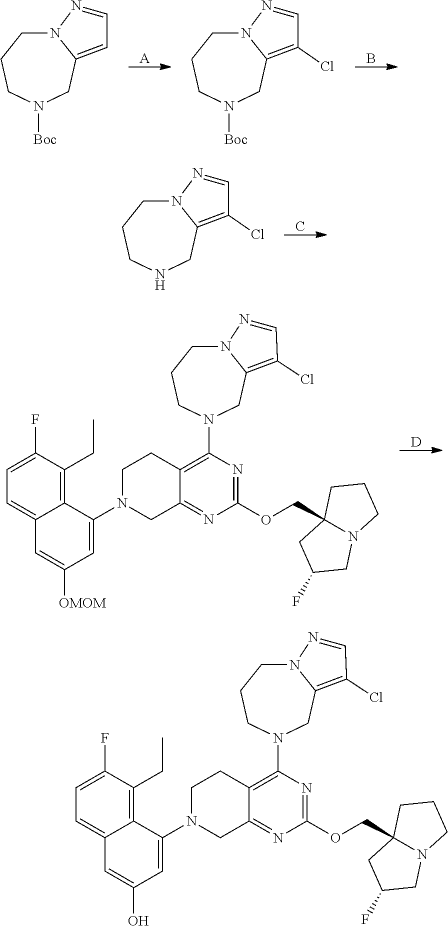

Step A. tert-butyl 3-chloro-7,8-dihydro-4H-pyrazolo[1,5-a][1,4]diazepine-5(6H)-carboxylate: To a solution of tert-butyl 7,8-dihydro-4H-pyrazolo[1,5-a][1,4]diazepine-5(6H)-carboxylate (500 mg, 1.0 equiv) in AcOH (5 mL) was added NCS (338 mg, 1.2 equiv). The reaction was stirred at 80° C. for 4 hours. The mixture was poured into saturated NaHCO₃ aqueous (50 mL) and extracted with ethyl acetate (3×20 mL). The combined organic layers were dried over anhydrous sodium sulfate, concentrated, and purified with reversed phase flash [C18, 0.1% formic acid condition] to afford the title compound (0.55 g, 92% yield) as a colorless oil. LCMS (ESI, M+1): m/z=272.1.

Step B. 3-chloro-5,6,7,8-tetrahydro-4H-pyrazolo[1,5-a][1,4]diazepine: To a solution of tert-butyl 3-chloro-7,8-dihydro-4H-pyrazolo[1,5-a][1,4]diazepine-5(6H)-carboxylate (550 mg, 1.0 equiv) in MeOH (2 mL) was added HCl-MeOH (4 M, 5 mL). The reaction was stirred at 20° C. for 2 hours. The mixture was concentrated. The residue was dissolved in MeOH (10 mL). To the mixture was added NaHCO₃solid (1.00 g). The reaction was stirred at 20° C. for 0.5 hours. The mixture was filtered. The filter cake was washed with (DCM/MeOH=10/1, 10 mL). The combined organic phase was concentrated to afford the title compound (0.368 g, crude) as a brown oil.

The last two steps were according to Example 248. The title compound was obtained as pink solid (FA salt). ¹H NMR (400 MHz, METHANOL-d₄) δ=7.52 (dd, J=5.6, 8.8 Hz, 1H), 7.39 (d, J=2.0 Hz, 1H), 7.15 (t, J=9.2 Hz, 1H), 7.01-6.94 (m, 2H), 5.57-5.35 (m, 1H), 4.99-4.93 (m, 1H), 4.82 (br s, 1H), 4.53-4.22 (m, 4H), 4.17-4.05 (m, 2H), 4.04-3.91 (m, 1H), 3.76-3.58 (m, 4H), 3.57-3.47 (m, 1H), 3.45-3.32 (m, 3H), 3.30-3.24 (m, 1H), 3.21-3.09 (m, 1H), 2.73 (br d, J=14.8 Hz, 1H), 2.64-2.29 (m, 3H), 2.28-1.91 (m, 5H), 1.10 (t, J=7.2 Hz, 3H); LCMS (ESI, M+1): m/z=650.4.

### Example 643

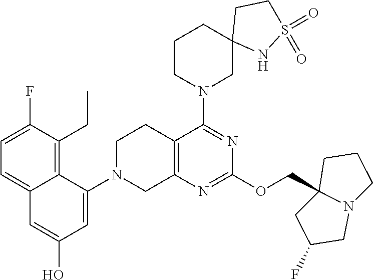 [SMILES: CCc1c(F)ccc2cc(O)cc(N3CCc4c(nc(OC[C@@]56CCCN5C[C@H](F)C6)nc4N4CCCC5(CCS(=O)(=O)N5)C4)C3)c12]

7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-2-thia-1,7-diazaspiro[4.5]decane 2,2-dioxide

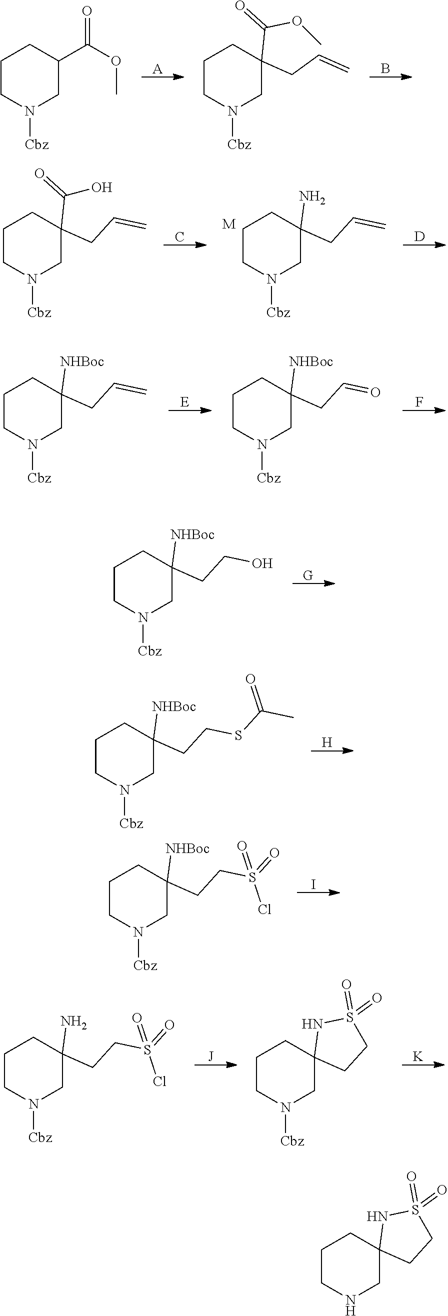

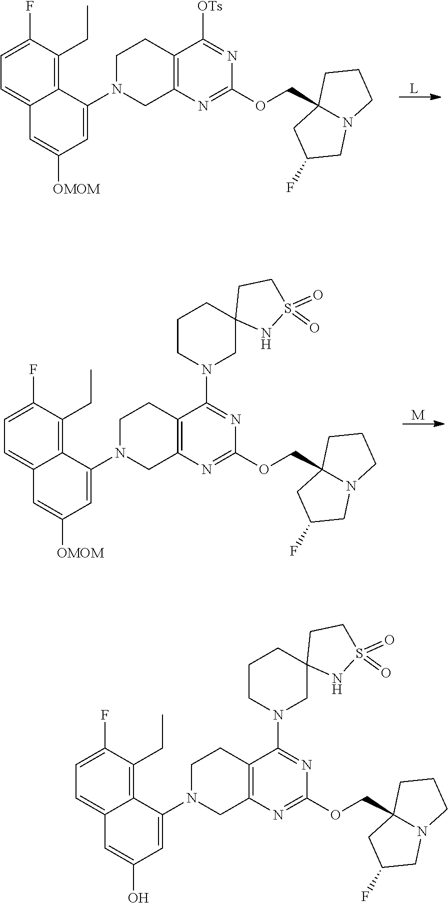

Step A. 1-benzyl 3-methyl 3-allylpiperidine-1,3-dicarboxylate: To a solution of 1-benzyl 3-methyl piperidine-1,3-dicarboxylate (50.0 g, 1.0 equiv) in THE (500 mL) was added LiHMDS (1 M, 234 mL, 1.3 equiv) at −78° C. in 30 minutes. The reaction was stirred at −78° C. for 1 hour. 3-bromoprop-1-ene (26.2 g, 1.2 equiv) was added into above mixture at −78° C. The reaction was stirred at 20° C. for 5 hours. The mixture was quenched by saturated NH₄Cl (200 mL) and extracted with ethyl acetate (3×200 mL). The combined mixture organic layers were washed with 10% aqueous citric acid (2×200 mL) and brine (200 mL), dried over anhydrous sodium sulfate, concentrated to afford the title compound (55.0 g, 96% yield) as brown oil;H NMR (400 MHz, CHLOROFORM-d) δ=7.47-7.28 (m, 5H), 5.81-5.54 (m, 1H), 5.19-5.00 (m, 4H), 4.00 (br d, J=13.2 Hz, 1H), 3.70-3.53 (m, 4H), 3.21 (br d, J=13.6 Hz, 2H), 2.40-2.27 (m, 1H), 2.27-2.16 (m, 1H), 2.05 (s, 1H), 1.60 (br d, J=4.0 Hz, 3H)

Step B. 3-allyl-1-((benzyloxy)carbonyl)piperidine-3-carboxylic acid: To a solution of 1-benzyl 3-methyl 3-allylpiperidine-1,3-dicarboxylate (55.0 g, 1.0 equiv) in MeOH (300 mL) and H₂O (100 mL) was added LiOH•H₂O (29.1 g, 4.0 equiv). The reaction was stirred at 60° C. for 12 hours. The mixture was adjusted pH to 2 by adding 2M HCl and extracted with ethyl acetate (500 mL). The organic layer was concentrated to afford the title compound (49.0 g, 93% yield) as yellow oil;

Step C. benzyl 3-allyl-3-aminopiperidine-1-carboxylate: To a mixture of 3-allyl-1-((benzyloxy)carbonyl)piperidine-3-carboxylic acid (49.0 g, 1.0 equiv) and DPPA (48.9 g, 1.1 equiv) in toluene (500 mL) was added TEA (32.7 g, 2.0 equiv). The reaction was stirred at 80° C. for 4 hours. The mixture was adjusted pH=2 with 4M HCl•dioxane. The mixture was diluted with water (150 mL) and separated. The water layer was adjusted pH to 9 with NaHCO₃solid and extracted with ethyl acetate (2×100 mL). The combined organic layer were washed with brine (200 mL), dried over sodium sulfate and concentrated to afford the title compound (43.0 g, 97% yield) as yellow oil, LCMS (ESI, M+1): m/z=275.2.

Step D. benzyl 3-allyl-3-((tert-butoxycarbonyl)amino)piperidine-1-carboxylate: A solution of benzyl 3-allyl-3-aminopiperidine-1-carboxylate (42.0 g, 1.0 equiv) and (Boc)20 (334 g, 10 equiv) was stirred at 60° C. for 4 hours. The mixture was concentrated and purified by column chromatography (SiO₂, Petroleum ether/Ethyl acetate=12/1) to afford the title compound (36.0 g, 62% yield) as white solid; LCMS (ESI, M-55): m/z=319.1.

Step E. benzyl 3-((tert-butoxycarbonyl)amino)-3-(2-oxoethyl)piperidine-1-carboxylate: To a solution of benzyl 3-allyl-3-((tert-butoxycarbonyl)amino)piperidine-1-carboxylate (4.00 g, 1.0 equiv) in THE (40 mL) and H₂O (40 mL) were added NaIO4 (4.57 g, 2.0 equiv) and potassium osmate dihydrate (197 mg, 0.05 equiv) at 0° C. The reaction was stirred at 20° C. for 2 hours. The mixture was quenched with saturated Na₂S₂O₃ aqueous solution (100 mL) and extracted with ethyl acetate (3×100 mL). The combined organic layers were concentrated and purified by column chromatography (SiO₂, Petroleum ether/Ethyl acetate=10/1 to 2/1) to afford the title compound (1.70 g, 41% yield) as white solid; ¹H NMR (400 MHz, CHLOROFORM-d) δ=9.79 (br s, 1H), 7.40-7.35 (m, 4H), 7.34-7.30 (m, 1H), 5.29-5.04 (m, 2H), 4.16-3.85 (m, 2H), 3.20-2.71 (m, 4H), 1.78-1.47 (m, 4H), 1.41 (s, 9H); LCMS (ESI, M-99): m/z=277.2.

Step F. benzyl 3-((tert-butoxycarbonyl)amino)-3-(2-hydroxyethyl)piperidine-1-carboxylate: To a solution of benzyl 3-((tert-butoxycarbonyl)amino)-3-(2-oxoethyl)piperidine-1-carboxylate (5.60 g, 1.0 equiv) in EtOH (30 mL) and THE (30 mL) was added NaBH4 (1.18 g, 2.1 equiv). The reaction was stirred at 0° C. for 1 hour. The mixture was quenched with sat. NH₄Cl (30 mL) slowly and extracted with ethyl acetated (100 mL). The combined organic layer was concentrated and purified by column chromatography (SiO2, Petroleum ether/Ethyl acetate=10/1 to 1/1) to afford the title compound (5.30 g, 93% yield) as white solid; ¹H NMR (400 MHz, CHLOROFORM-d) δ=7.41-7.35 (m, 4H), 7.35-7.29 (m, 1H), 5.28-5.05 (m, 2H), 4.98-4.65 (m, 1H), 4.11-3.96 (m, 1H), 3.90-3.63 (m, 3H), 3.14-2.94 (m, 2H), 2.65-2.33 (m, 1H), 1.74-1.45 (m, 4H), 1.42 (s, 9H); LCMS (ESI, M-99): m/z=279.1.

Step G. benzyl 3-(2-(acetylthio)ethyl)-3-((tert-butoxycarbonyl)amino)piperidine-1-carboxylate: To a mixture of PPh3 (3.05 g, 2.0 equiv) in THE (40 mL) was added DIAD (2.35 g, 2.0 equiv) at 0° C. The reaction was stirred at 0° C. for 0.5 hour. A solution of benzyl 3-((tert-butoxycarbonyl)amino)-3-(2-hydroxyethyl)piperidine-1-carboxylate (2.20 g, 1.0 equiv) and ethanethioic S-acid (885 mg, 2.0 equiv) in THE (20 mL) was added into above mixture at 0° C. The reaction was stirred at 0° C. for 1 hour and 15° C. for 1 hour. The mixture was diluted with water (100 mL) and extracted with ethyl acetate (2×100 mL). The combined organic layers were concentrated and purified by column chromatography (SiO2, Petroleum ether/Ethyl acetate=20/1 to 7/1) to afford the title compound (5.00 g, crude) as white solid; LCMS (ESI, M-99): m/z=337.1.

Step H. benzyl 3-((tert-butoxycarbonyl)amino)-3-(2-(chlorosulfonyl)ethyl)piperidine-1-carboxylate: To a solution of benzyl 3-(2-(acetylthio)ethyl)-3-((tert-butoxycarbonyl)amino)piperidine-1-carboxylate (1.00 g, 1.0 equiv) in AcOH (6 mL) and H₂O (0.6 mL) was added NCS (918 mg, 3.0 equiv). The reaction was stirred at 15° C. for 0.5 hours. The mixture was diluted with water (50 mL) and extracted with ethyl acetate (50 mL). The organic phase was dried over anhydrous sodium sulfate and concentrated to afford the title compound (1.5 g, crude) as yellow oil.

Step I. benzyl 3-amino-3-(2-(chlorosulfonyl)ethyl)piperidine-1-carboxylate: To a solution of benzyl 3-((tert-butoxycarbonyl)amino)-3-(2-(chlorosulfonyl)ethyl)piperidine-1-carboxylate (1.50 g, 1.0 equiv) in DCM (2 mL) was added TFA (7.70 g, 21 equiv) at 0° C. The reaction was stirred at 0° C. for 15 minutes. The mixture was concentrated to afford the title compound (1.2 g, crude) as yellow oil.

Step J. benzyl 2-thia-1,7-diazaspiro[4.5]decane-7-carboxylate 2,2-dioxide: To a solution of benzyl 3-amino-3-(2-(chlorosulfonyl)ethyl)piperidine-1-carboxylate (1.2 g, crude) in THF(12 mL) was added K₂CO₃ (2.00 g, 14.5 mmol). The reaction was stirred at 15° C. for 2 hours. The mixture was added water (10 mL) and extracted with ethyl acetate (20 mL). The organic layer was dried over anhydrous sodium sulfate, concentrated, and purified with reversed phase flash [C18, 0.1% formic acid condition] and column chromatography (SiO₂, Petroleum ether/Ethyl acetate=5/1 to 1/2) to afford the title compound (100 mg, 26% yield over four steps) as yellow oil; 1H NMR (400 MHz, CiLOROFORM-d) δ=7.42-7.29 (m, 5H), 5.23-5.06 (m, 2H), 4.5 5-4.24 (m, 1H), 3.72-3.29 (m, 4H), 3.18 (br s, 2H), 2.37-2.09 (m, 2H), 1.89 (br s, 1H), 1.78-1.63 (m, 3H); LCMS (ESI, M+1): m/z=325.2.

Step K. 2-thia-1,7-diazaspiro[4.5]decane 2,2-dioxide: To a solution of benzyl 2-thia-1,7-diazaspiro[4.5]decane-7-carboxylate 2,2-dioxide (100 mg, 1.0 equiv) in MeOH (2 mL) was added Pd/C (30.0 mg, 10% purity) under N₂. The reaction was degassed and purged with H₂ 3 times. The reaction was stirred under H₂ (15 psi) at 15° C. for 3 hours. The mixture was filtered and concentrated to afford the title compound (70 mg, crude) as yellow gum.

Step L. 7-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d] pyrimidin-4-yl)-2-thia-1,7-diazaspiro[4.5]decane 2,2-dioxide: To a solution of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (200 mg, 1.0 equiv) and 2-thia-1,7-diazaspiro[4.5]decane 2,2-dioxide (82.1 mg, 1.5 equiv) in DMF (1 mL) was added DIEA (112 mg, 3.0 equiv). The reaction was stirred at 40° C. for 12 hours. The mixture was filtered and purified by reversed phase flash [C18, 0.1% formic acid condition] to afford the title compound (180 mg, 88% yield) as yellow solid; LCMS (ESI, M+1): m/z=713.3.

Step M. 7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d] pyrimidin-4-yl)-2-thia-1,7-diazaspiro[4.5]decane 2,2-dioxide: To a solution of 7-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-2-thia-1,7-diazaspiro[4.5]decane 2,2-dioxide (170 mg, 1.0 equiv) in MeOH (1 mL) was added HCl•MeOH (4 M, 3.58 mL, 60 equiv) at 0° C. The reaction was stirred at 0° C. for 0.5 hours. The mixture was concentrated. The residue was diluted with water (1 mL). The mixture was adjusted to pH=9 with saturated NaHCO₃ (3 mL) and extracted with ethyl acetate (2×5 mL). The combined organic layers were concentrated and purified by prep-HPLC [column: Phenomenex luna C18 150×25 mm×10 μm; A: water (FA), B: ACN, B %: 21%-51% over 10 min] to afford the title compound (81.1 mg, 48% yield, 0.71 HCOOH) as yellow solid; ¹H NMR (400 MHz, METHANOL-d₄) δ=7.51 (td, J=5.2, 9.2 Hz, 1H), 7.14 (dt, J=3.2, 9.2 Hz, 1H), 7.03-6.90 (m, 2H), 5.57-5.36 (m, 1H), 4.52-4.29 (m, 2H), 4.20-3.77 (m, 3H), 3.76-3.53 (m, 5H), 3.53-3.32 (m, 4H), 3.30-3.06 (m, 6H), 2.72-2.45 (m, 2H), 2.43-2.25 (m, 3H), 2.24-2.15 (m, 2H), 2.13-2.02 (m, 1H), 2.00-1.68 (m, 4H), 1.10 (dt, J=4.0, 7.2 Hz, 3H); LCMS (ESI, M+1): m/z=669.2.

### Example 644

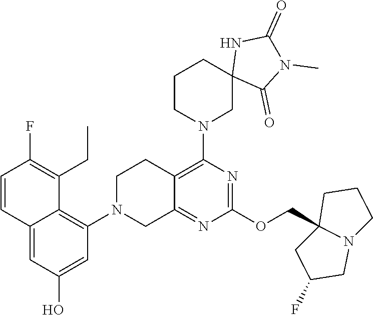 [SMILES: CCc1c(F)ccc2cc(O)cc(N3CCc4c(nc(OC[C@@]56CCCN5C[C@H](F)C6)nc4N4CCCC5(C4)NC(=O)N(C)C5=O)C3)c12]

### 7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-3-methyl-1,3,7-triazaspiro[4.5]decane-2,4-dione

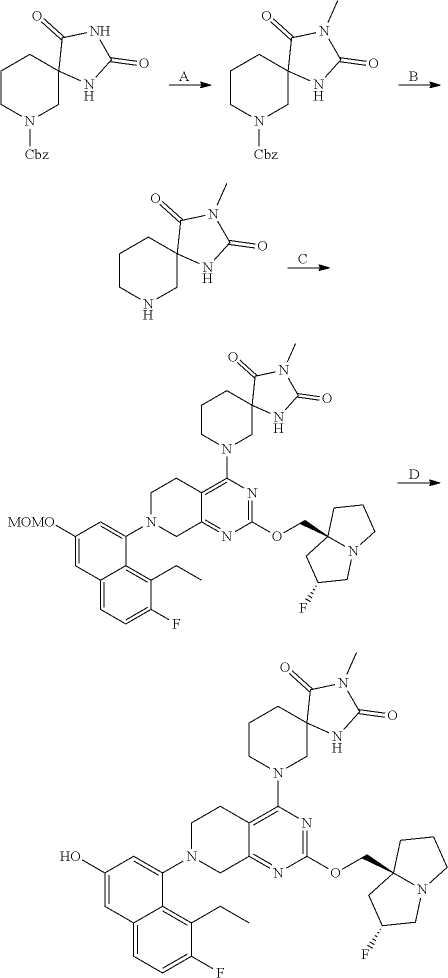

Step A. benzyl 3-methyl-2,4-dioxo-1,3,7-triazaspiro[4.5]decane-7-carboxylate: To a solution of benzyl 2,4-dioxo-1,3,7-triazaspiro[4.5]decane-7-carboxylate (200 mg, 1.0 equiv) in DMSO (2 mL) was added K₂CO₃ (273 mg, 3.0 equiv) and CH₃I (74.9 mg, 0.80 equiv). The reaction was stirred at 25° C. for 1 hour. The mixture was filtered and purified by reversed-phase HPLC (0.1% FA condition) to afford the title compound (80.0 mg, 33% yield) as yellow solid; LCMS (ESI, M+1): m/z=318.1.

Step B. 3-methyl-1,3,7-triazaspiro[4.5]decane-2,4-dione: To a mixture of benzyl 3-methyl-2,4-dioxo-1,3,7-triazaspiro[4.5]decane-7-carboxylate (70.0 mg, 1.0 equiv) in NH₃MeOH (1 mL, 20% purity) and MeOH (1 mL) was added Pd/C (10.0 mg, 10% purity) under N₂ atmosphere. The reaction was degassed and purged with H₂ 3 times. The reaction was stirred under H₂ (15 Psi) at 25° C. for 1 hour. The mixture was filtered and concentrated to give the title compound (76.0 mg, crude) as white solid; LCMS (ESI, M+1): m/z=184.0.

The last two steps were according to Example 248. The title compound was obtained as orange solid (FA salt). ¹H NMR (400 MHz, METHANOL-d₄) δ=7.51 (dd, J=6.0, 9.2 Hz, 1H), 7.15 (t, J=9.6 Hz, 1H), 7.04-6.91 (m, 2H), 5.53-5.25 (m, 1H), 4.39-4.19 (m, 2H), 4.18-3.95 (m, 3H), 3.66 (br dd, J=6.4, 17.6 Hz, 1H), 3.58-3.42 (m, 5H), 3.40 (br s, 2H), 3.25-3.12 (m, 3H), 3.10-2.90 (m, 4H), 2.84-2.68 (m, 1H), 2.35 (br s, 2H), 2.25-2.07 (m, 4H), 2.05-1.80 (m, 4H), 1.10 (br t, J=7.2 Hz, 3H); LCMS (ESI, M+1): m/z=662.4.

### Example 645

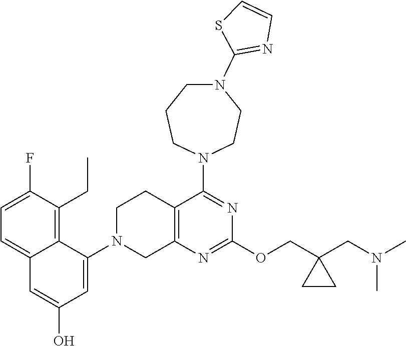 [SMILES: CCc1c(F)ccc2cc(O)cc(N3CCc4c(nc(OCC5(CN(C)C)CC5)nc4N4CCCN(c5nccs5)CC4)C3)c12]

### 4-(2-((1-((dimethylamino)methyl)cyclopropyl)methoxy)-4-(4-(thiazol-2-yl)-1,4-diazepan-1-yl)-5,8-dihydropyrido[3,4-d]pyrimidin-7(6H)-yl)-5-ethyl-6-fluoronaphthalen-2-ol

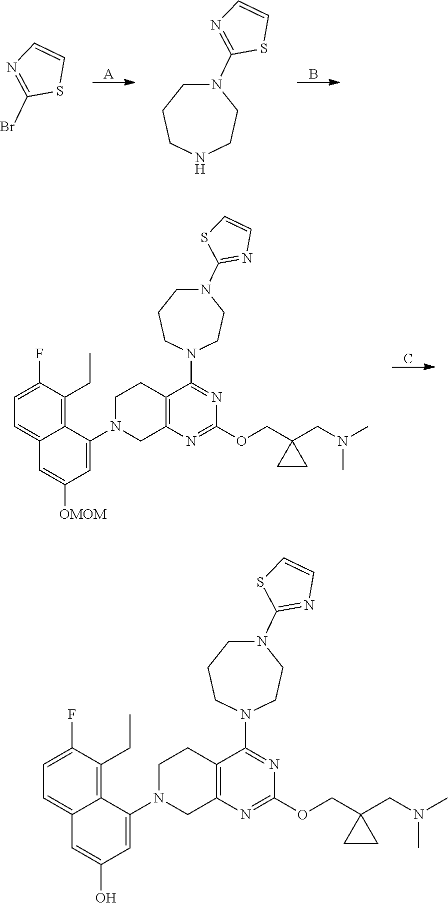

Step A. 2-(1,4-diazepan-1-yl)thiazole: To a solution of 2-bromothiazole (1.00 g, 1 equiv) in n-BuOH (13 mL) was added 4-diazepane (1.22 g, 2 equiv). The reaction was stirred at 118° C. for 12 hours. The mixture was diluted with water (20 mL) and extracted with ethyl acetate (10 mL×3). The combined organic layers were washed with brine, dried over anhydrous sodium sulfate, filtered and concentrated under reduced pressure to afford the title compound (900 mg, crude) as a yellow oil; LCMS (ESI, M+1): m/z=184.2.

The last two steps were according to Example 594. The title compound was obtained as yellow solid (FA salt). ¹H NMR (400 MHz, METHANOL-d₄) δ=7.51 (dd, J=7.0, 8.8 Hz, 1H), 7.14 (t, J=9.6 Hz, 1H), 7.08 (d, J=3.6 Hz, 1H), 7.01-6.93 (m, 2H), 6.61 (d, J=3.6 Hz, 1H), 4.19 (s, 2H), 4.08-4.03 (m, 2H), 4.02 (s, 1H), 3.92-3.87 (m, 1H), 3.87-3.82 (m, 2H), 3.80 (br t, J=6.0 Hz, 2H), 3.65 (br d, J=17.6 Hz, 1H), 3.58 (td, J=4.4, 15.2 Hz, 1H), 3.50 (br d, J=8.4 Hz, 1H), 3.43-3.33 (m, 2H), 3.24-3.06 (m, 2H), 2.77-2.70 (m, 1H), 2.68 (br s, 2H), 2.48 (br s, 6H), 2.35-2.22 (m, 1H), 1.99-1.88 (m, 1H), 1.12 (t, J=7.6 Hz, 3H), 0.73 (s, 2H), 0.59 (br s, 2H); LCMS (ESI, M+1): m/z=632.2.

### Example 646

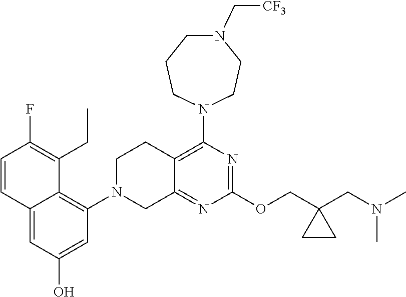 [SMILES: CCc1c(F)ccc2cc(O)cc(N3CCc4c(nc(OCC5(CN(C)C)CC5)nc4N4CCCN(CC(F)(F)F)CC4)C3)c12]

### 4-(2-((1-((dimethylamino)methyl)cyclopropyl)methoxy)-4-(4-(2,2,2-trifluoroethyl)-1,4-diazepan-1-yl)-5,8-dihydropyrido[3,4-d]pyrimidin-7(6H)-yl)-5-ethyl-6-fluoronaphthalen-2-ol

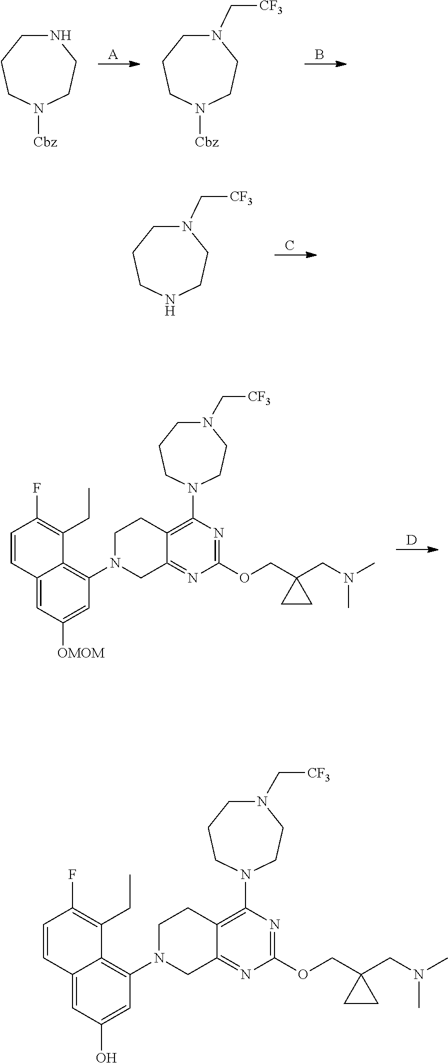

Step A. benzyl 4-(2,2,2-trifluoroethyl)-1,4-diazepane-1-carboxylate: To a mixture of benzyl 1,4-diazepane-1-carboxylate (1.00 g, 1.0 equiv) and K₂CO₃ (1.77 g, 3 equiv) in DMF (10 mL) was added trifluoroethyl trifluoromethanesulfonate (1.98 g, 2.00 equiv). The reaction was stirred at 20° C. for 17 hours under N₂ atmosphere. The mixture was concentrated under reduced pressure and purified by reversed phase flash chromatography [C18, 0.1% formic acid condition] to afford the title compound (700 mg, 37.3% yield) as yellow solid; LCMS (ESI, M+1): m/z=317.1.

Step B 1-(2,2,2-trifluoroethyl)-1,4-diazepane: A mixture of benzyl 4-(2,2,2-trifluoroethyl)-1,4-diazepane-1-carboxylate (700 mg, 1.0 equiv) and Pd/C (200 mg, 0.1 equiv) in MeOH (40.0 mL) was degassed and purged with H₂ for 3 times. The reaction was stirred at 20° C. for 5 hours under H₂ atmosphere (15 Psi). The mixture was filtered and concentrated under reduced pressure to afford the title compound (380 mg, crude) as colorless oil; LCMS (ESI, M+1): m/z=183.1.

The last two steps were according to Example 594. The title compound was obtained as yellow solid; ¹H NMR (400 MHz, METHANOL-d₄) δ=7.52 (dd, J=6.0, 9.2 Hz, 1H), 7.15 (t, J=9.4 Hz, 1H), 7.00 (d, J=2.4 Hz, 1H), 6.97 (d, J=2.4 Hz, 1H), 4.27-4.14 (m, 2H), 4.04 (br d, J=17.2 Hz, 1H), 3.98-3.82 (m, 3H), 3.82-3.73 (m, 1H), 3.65 (d, J=17.5 Hz, 1H), 3.53-3.47 (m, 1H), 3.44-3.36 (m, 2H), 3.27 (dt, J=3.8, 9.6 Hz, 2H), 3.23-3.15 (m, 3H), 3.09-3.00 (m, 1H), 2.99-2.86 (m, 2H), 2.77-2.68 (m, 1H), 2.49-2.37 (m, 2H), 2.30 (s, 6H), 2.15-2.03 (m, 1H), 1.93-1.80 (m, 1H), 1.12 (t, J=7.3 Hz, 3H), 0.71-0.63 (m, 2H), 0.54-0.44 (m, 2H); F NMR (400 MHz, METHANOL-d₄) δ=−72, -123; LCMS (ESI, M+1): m/z=631.3.

### Example 647

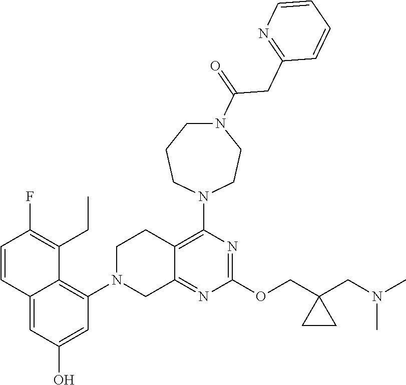 [SMILES: CCc1c(F)ccc2cc(O)cc(N3CCc4c(nc(OCC5(CN(C)C)CC5)nc4N4CCCN(C(=O)Cc5ccccn5)CC4)C3)c12]

### 1-(4-(2-((1-((dimethylamino)methyl)cyclopropyl)methoxy)-7-(8-ethyl-7-fluoro-3-hydroxynaphthalen-1-yl)-5,6,7,8-tetrahydropyrido[3,4-d]pyrimidin-4-yl)-1,4-diazepan-1-yl)-2-(pyridin-2-yl)ethan-1-one

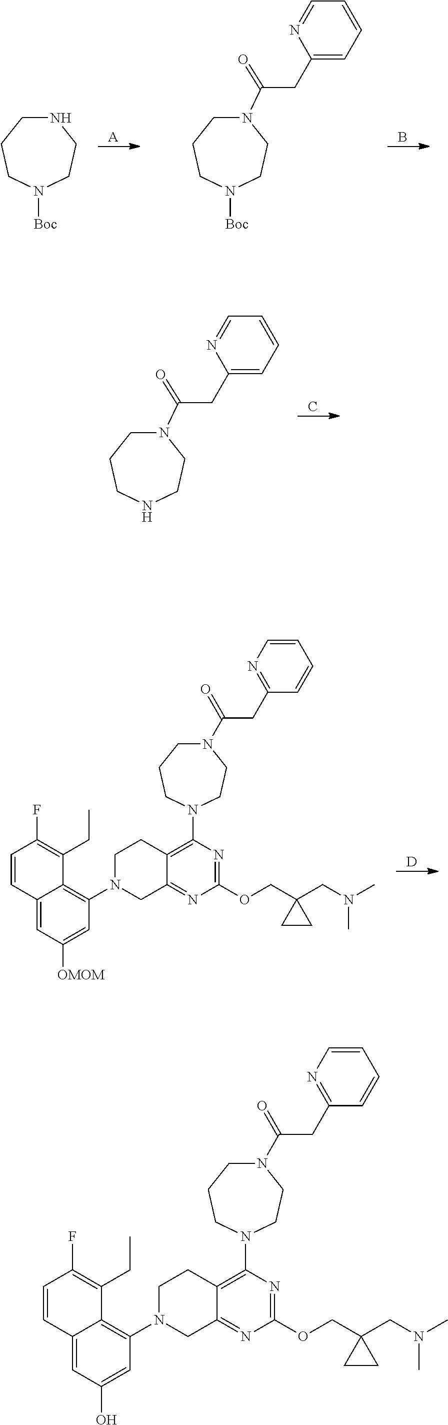

Step A. tert-butyl 4-(2-(pyridin-2-yl)acetyl)-1,4-diazepane-1-carboxylate: To a mixture of tert-butyl 1,4-diazepane-1-carboxylate (1.00 g, 1.0 equiv) and 2-(pyridin-2-yl)acetic acid (684.73 mg, 1.0 equiv) in DCM (10 mL) were added TEA (1.52 g, 3.0 equiv) and HATU (1.90 g, 1.0 equiv) in one portion under N₂. The reaction was stirred at 20° C. for 3 hours. The mixture was diluted with water (20 mL) and extracted with ethyl acetate (3×30 mL). The combined organic layers were washed with brine (30 mL), dried over Na₂SO₄, filtered, concentrated under reduced pressure and purified by reversed phase flash chromatography [C18, 0.1% formic acid condition] to afford the title compound (1.27 g, 79% yield) as yellow oil.

Step B. 1-(1,4-diazepan-1-yl)-2-(pyridin-2-yl)ethan-1-one: A mixture of tert-butyl 4-(2-(pyridin-2-yl)acetyl)-1,4-diazepane-1-carboxylate (1.25 g, 1.0 equiv) in HCl•MeOH (4 M, 978 μL, 1.0 equiv) were stirred at 0° C. for 1 hour. The mixture was concentrated under reduced pressure, dissolve in MeOH (5 ml), adjusted to pH=8 with saturated NaHCO₃, filtered and concentrated to afford the title compound (850 mg, 97% yield) as a yellow oil; ¹H NMR (400 MHz, DMSO-d₆) δ=8.55-8.50 (m, 1H), 7.85 (t, J=7.6 Hz, 1H), 7.41 (d, J=7.8 Hz, 1H), 7.38-7.31 (m, 1H), 3.99-3.95 (m, 2H), 3.86 (br t, J=5.2 Hz, 1H), 3.70-3.67 (m, 1H), 3.65 (t, J=6.1 Hz, 1H), 3.55 (br t, J=6.0 Hz, 1H), 3.42-3.32 (m, 2H), 3.25 (m, 1H), 3.11 (m, 1H), 2.13-1.88 (m, 2H); LCMS (ESI, M+1): m/z=219.9.

The last two steps were according to Example 594. The title compound was obtained as an orange solid (¹H NMR (400 MHz, DMSO-d₆) δ=8.43 (d, J=2.4 Hz, 1H), 8.17 (s, 1H), 7.72-7.62 (m, 1H), 7.62-7.55 (m, 1H), 7.27-7.17 (m, 3H), 6.99 (s, 2H), 4.03 (d, J=6.0 Hz, 2H), 3.94-3.84 (m, 4H), 3.82-3.76 (m, 4H), 3.72-3.63 (m, 4H), 3.13-3.01 (m, 4H), 2.64-2.56 (m, 2H), 2.22 (br d, J=5.6 Hz, 2H), 2.17 (s, 3H), 2.14 (s, 3H), 1.94-1.81 (m, 1H), 1.74-1.61 (m, 1H), 1.08-0.99 (m, 3H), 0.55 (m, 2H), 0.36 (m, 2H); LCMS (ESI, M+1): m/z=668.3.

### Example 648

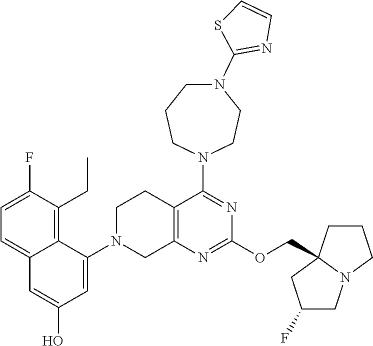 [SMILES: CCc1c(F)ccc2cc(O)cc(N3CCc4c(nc(OC[C@@]56CCCN5C[C@H](F)C6)nc4N4CCCN(c5nccs5)CC4)C3)c12]

### 5-ethyl-6-fluoro-4-(2-(((2R,7aS)-2-fluorotetrahydro-1H-pyrrolizin-7a(5H)-yl)methoxy)-4-(4-(thiazol-2-yl)-1,4-diazepan-1-yl)-5,8-dihydropyrido[3,4-d]pyrimidin-7(6H)-yl)naphthalen-2-ol

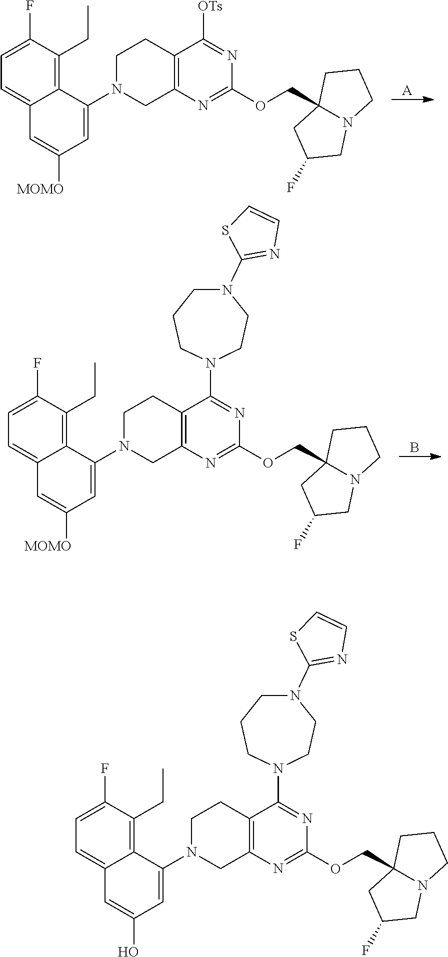

Synthesized according to Example 248. The title compound was obtained as an orange solid (FA salt). ¹H NMR (400 MHz, METHANOL-d₄) δ=7.55-7.50 (m, 1H), 7.16 (t, J=9.4 Hz, 1H), 7.10-7.07 (m, 1H), 7.00-6.97 (m, 2H), 6.62 (t, J=3.7 Hz, 1H), 5.52-5.35 (m, 1H), 4.38-4.32 (m, 1H), 4.29-4.23 (m, 1H), 4.12-4.00 (m, 3H), 3.91-3.80 (m, 5H), 3.71-3.54 (m, 6H), 3.40-3.35 (m, 2H), 3.27-3.14 (m, 3H), 2.78-2.70 (m, 1H), 2.55-2.33 (m, 3H), 2.27-2.15 (m, 3H), 2.08-1.91 (m, 2H), 1.16-1.10 (m, 3H); LCMS (ESI, M+1): m/z=662.3.

### Example 649

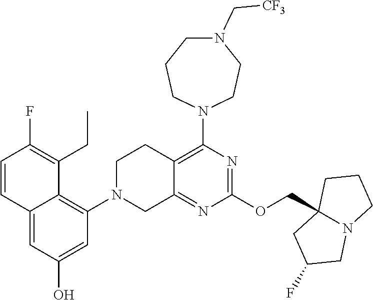 [SMILES: CCc1c(F)ccc2cc(O)cc(N3CCc4c(nc(OC[C@@]56CCCN5C[C@H](F)C6)nc4N4CCCN(CC(F)(F)F)CC4)C3)c12]

### 5-ethyl-6-fluoro-4-(2-(((2R,7aS)-2-fluorotetrahydro-1H-pyrrolizin-7a(5H)-yl)methoxy)-4-(4-(2,2,2-trifluoroethyl)-1,4-diazepan-1-yl)-5,8-dihydropyrido[3,4-d]pyrimidin-7(6H)-yl)naphthalen-2-ol

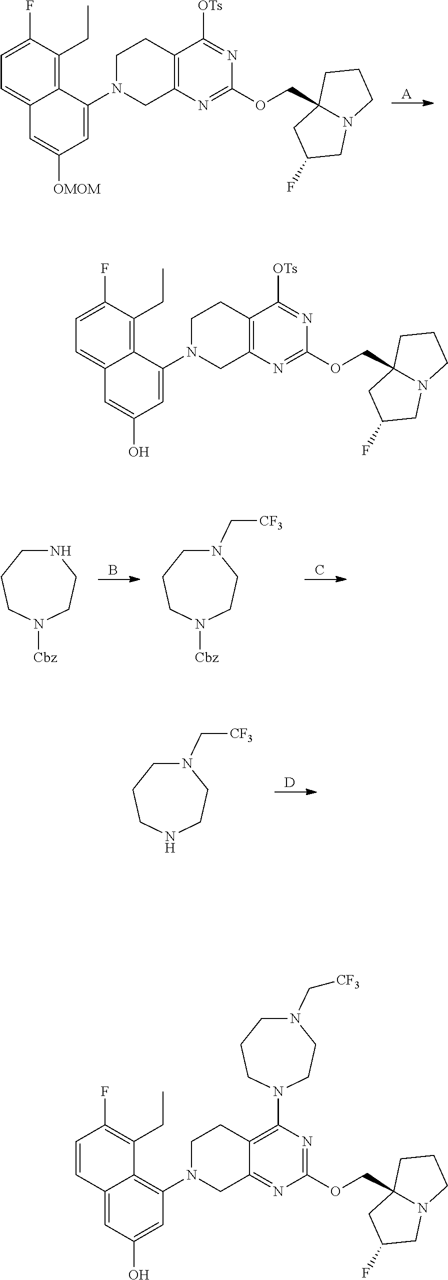

Step A. 7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate: To a mixture of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (250 mg, 1.0 equiv) in DCM (2.5 mL) was added TFA (205 mg, 5.0 equiv) in one portion at 0° C. under N₂. The reaction was stirred at 0° C. for 4 hours. The mixture was concentrated, diluted with water (4 mL), adjusted to pH=8 with saturated NaHCO₃ aqueous solution and extracted with DCM (3×15 mL). The combined organic phase was washed with brine (15 mL), dried over Na₂SO₄, filtered, concentrated and purified by reversed phase flash chromatography [C18, 0.1% formic acid condition] to afford the title compound (150 mg, 53% yield) as yellow solid; LCMS (ESI, M+1): m/z=651.3.

Step B. benzyl 4-(2,2,2-trifluoroethyl)-1,4-diazepane-1-carboxylate: To a mixture of 2,2,2-trifluoroethyl trifluoromethanesulfonate (1.94 g, 2.0 equiv) and benzyl 1,4-diazepane-1-carboxylate (980 mg, 1.0 equiv) in DMF (10 mL) was added K₂CO₃ (1.73 g, 3.0 equiv) in one portion under N₂. The reaction was stirred at 20° C. for 4 hours. The mixture was diluted with water (4 mL), adjusted to pH=8 with saturated NaHCO₃ aqueous solution and extracted with DCM (15 mL×3). The combined organic phase was washed with brine (15 mL), dried over Na₂SO₄, filtered, concentrated and purified by reversed phase flash chromatography [C18, 0.1% formic acid condition] to afford the title compound (1.2 g, 89% yield) as yellow oil liquid; LCMS (ESI, M+1): m/z=317.2.

Step C. 1-(2,2,2-trifluoroethyl)-1,4-diazepane: To a mixture of benzyl 4-(2,2,2-trifluoroethyl)-1,4-diazepane-1-carboxylate (700 mg, 1.0 equiv) in MeOH (7 mL) was added Pd/C (200 mg, 10% purity, 1.0 equiv) in one portion under N₂. The reaction was stirred at 20° C. for 3 hours under H₂ (15 Psi). The mixture was filtered and concentrated under reduced pressure to afford the title compound (319 mg, 79% yield) as white solid. ¹H NMR (400 MHz, METHANOL-d₄) δ=3.31-3.25 (m, 2H), 2.98-2.91 (m, 8H), 1.83-1.77 (m, 2H).

Step D. 5-ethyl-6-fluoro-4-(2-(((2R,7aS)-2-fluorotetrahydro-1H-pyrrolizin-7a(5H)-yl)methoxy)-4-(4-(2,2,2-trifluoroethyl)-1,4-diazepan-1-yl)-5,8-dihydropyrido[3,4-d] pyrimidin-7(6H)-yl)naphthalen-2-ol: To a mixture of 7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (50 mg, 76.84 μmol, 1 equiv) and 1-(2,2,2-trifluoroethyl)-1,4-diazepane (14.0 mg, 1.0 equiv) in DMF (0.5 mL) were added DIEA (29.8 mg, 3.0 equiv) and 4 Å molecular sieve (30.0 mg, 1.0 equiv) in one portion under N₂. The reaction was stirred at 40° C. for 12 hours. The mixture was filtered and purified by prep-HPLC [column: Phenomenex luna C18 150×25 mm×10 μm; mobile phase: water (FA)-ACN; B %: 33%-53%, 58 min] to afford the title compound (6.37 mg, 12% yield, HCOOH) as yellow solid. ¹H NMR (400 MHz, METHANOL-d₄) δ=7.54-7.49 (m, 1H), 7.14 (t, J=9.4 Hz, 1H), 6.99-6.97 (m, 1H), 6.97-6.95 (m, 1H), 5.41-5.26 (m, 1H), 4.26-4.11 (m, 2H), 4.03 (m, 1H), 3.98-3.91 (m, 1H), 3.90-3.84 (m, 2H), 3.80-3.71 (m, 1H), 3.65 (m, 1H), 3.55-3.47 (m, 2H), 3.42-3.36 (m, 4H), 3.28-3.22 (m, 2H), 3.18 (m, 2H), 3.16-3.07 (m, 2H), 2.98-2.87 (m, 2H), 2.78-2.72 (m, 1H), 2.42-2.12 (m, 4H), 2.10-2.02 (m, 3H), 1.98-1.81 (m, 2H), 1.12-1.08 (m, 3H); F NMR (400 MHz, METHANOL-d₄) δ=−72.48, -123.05, -173.72; LCMS (ESI, M+1): m/z=661.2.

### Example 650

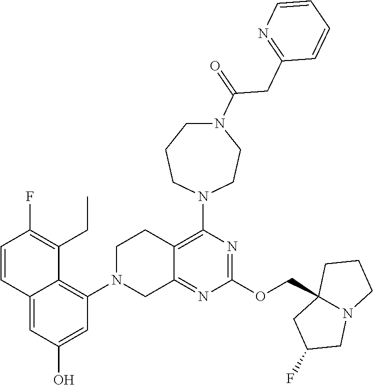 [SMILES: CCc1c(F)ccc2cc(O)cc(N3CCc4c(nc(OC[C@@]56CCCN5C[C@H](F)C6)nc4N4CCCN(C(=O)Cc5ccccn5)CC4)C3)c12]

### 1-(4-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,4-diazepan-1-yl)-2-(pyridin-2-yl)ethan-1-one

Synthesized according to Example 248. The title compound was obtained as an orange solid. ¹H NMR (400 MHz, DMSO-d₆) δ=8.43 (br s, 1H), 8.15 (s, 1H), 7.72-7.62 (m, 1H), 7.61-7.56 (m, 1H), 7.26-7.15 (m, 3H), 6.99 (s, 2H), 5.34-5.14 (m, 1H), 3.94-3.89 (m, 2H), 3.88-3.82 (m, 4H), 3.80 (br s, 4H), 3.68-3.60 (m, 4H), 3.07 (m, 2H), 2.99 (m, 2H), 2.86-2.75 (m, 2H), 2.69-2.56 (m, 2H), 2.10-2.04 (m, 1H), 2.02-1.90 (m, 3H), 1.86-1.77 (m, 2H), 1.77-1.61 (m, 4H), 1.08-0.99 (m, 3H); F NMR (400 MHz, DMSO-d₆) δ=−121, -172; LCMS (ESI, M+1): m/z=698.3.

### Example 651

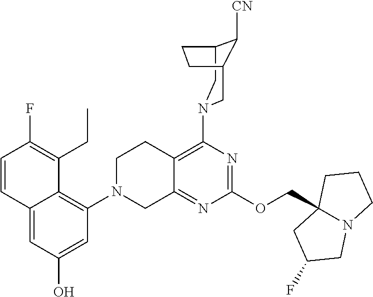 [SMILES: CCc1c(F)ccc2cc(O)cc(N3CCc4c(nc(OC[C@@]56CCCN5C[C@H](F)C6)nc4N4CC5CCC(C4)C5C#N)C3)c12]

### (1R,5S,8S)-3-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-3-azabicyclo[3.2.1]octane-8-carbonitrile

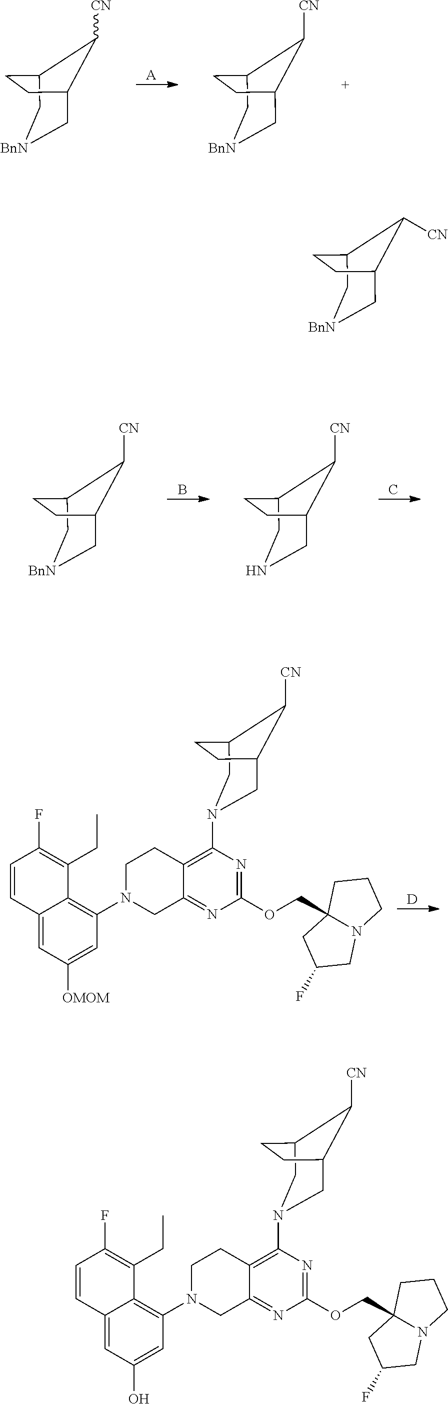

Step A. (1R,5S)-3-benzyl-3-azabicyclo[3.2.1]octane-8-carbonitrile (4.00 g) was purified by column chromatography (SiO₂, petroleum ether/ethyl acetate 20/1 to 10/1) and SFC separation [column: DAICEL CHIRALPAK IG 250 mm×50 mm,10 m; A: CO₂; B: EtOH (0.1% NH₃.H₂O), B %-: 10% over 4.5 min] to afford two isomers.

(1R,5S,8r)-3-benzyl-3-azabicyclo[3.2.1]octane-8-carbonitrile (1.70 g, 42% yield) as white solid; SFC: 97% ee, Column: Chiralpak IG-3 50×4.6 mm I.D.,3 m; mobile phase: 5%-40% EtOH (0.05% DEA) in CO₂, flow rate:3 mL/min; detector: 220 nm, t_{R}: 0.797 min; ¹H NMR (400 MHz, METHANOL-d₄) δ=7.23-7.08 (m, 5H), 3.36 (s, 2H), 2.59 (dd, J=4.2, 11.6 Hz, 2H), 2.52 (s, 1H), 2.32 (br s, 2H), 2.01 (s, 1H), 1.99 (s, 1H), 1.82-1.73 (m, 4H); LCMS (ESI, M+1): m/z=227.1.

(1R,5S,8s)-3-benzyl-3-azabicyclo[3.2.1]octane-8-carbonitrile (1.05 g, 26% yield) as white solid; ¹H NMR (400 MHz, METHANOL-d₄) δ=7.35-7.18 (m, 5H), 3.52 (s, 2H), 2.76-2.71 (m, 1H), 2.67-2.59 (m, 2H), 2.47 (d, J=11.6 Hz, 2H), 2.35 (br d, J=1.6 Hz, 2H), 1.91-1.81 (m, 2H), 1.75-1.64 (m, 2H); LCMS (ESI, M+1): m/z=227.1; SFC: >99% ee, Column: Chiralpak

IG-3 50×4.6 mm I.D.,3 μm; mobile phase: 5%-40% EtOH (0.05% DEA) in CO₂, flow rate:3 mL/min; detector: 220 nm, t_{R}: 0.906 min.

Step B. (1R,5S,8r)-3-azabicyclo[3.2.1]octane-8-carbonitrile: To a solution of (1R,5S,8r)-3-benzyl-3-azabicyclo[3.2.1]octane-8-carbonitrile (500 mg, 1.0 equiv) in MeOH (10.0 mL) was added Pd/C (50 mg, 10% purity) and AcOH (265 mg, 2.0 equiv) under N₂ atmosphere. The suspension was degassed and purged with H₂ for 3 times. The reaction was stirred at 25° C. for 3 hours under H₂ (15 Psi). The mixture was filtered and the filtrate was concentrated to afford the title compound (670 mg, crude, AcOH) as colorless oil; ¹H NMR (400 MHz, METHANOL-d₄) δ=3.20-3.07 (m, 4H), 2.84 (br d, J=7.2 Hz, 1H), 2.71-2.65 (m, 1H), 2.48-2.27 (m, 1H), 2.19 (br d, J=3.2 Hz, 2H), 1.92-1.69 (m, 2H).

The last two steps were according to Example 248. The title compound was obtained as white solid. ¹H NMR (400 MHz, DMSO-d₆) δ=9.75-9.64 (m, 1H), 7.59 (dd, J=6.0, 9.0 Hz, 1H), 7.24 (t, J=9.6 Hz, 1H), 6.98 (s, 2H), 5.43-4.95 (m, 1H), 4.12-4.02 (m, 1H), 3.98-3.92 (m, 1H), 3.92-3.88 (m, 1H), 3.81 (dd, J=2.8, 10.4 Hz, 1H), 3.63 (br d, J=17.2 Hz, 2H), 3.50-3.37 (m, 3H), 3.30-3.23 (m, 2H), 3.21-3.15 (m, 1H), 3.14-3.01 (m, 4H), 2.99-2.85 (m, 2H), 2.84-2.75 (m, 1H), 2.68-2.56 (m, 3H), 2.10-1.96 (m, 2H), 1.95-1.78 (m, 4H), 1.77-1.57 (m, 3H), 1.05 (dt, J=4.0, 7.2 Hz, 3H); LCMS (ESI, M+1): m/z=615.4.

### Example 652

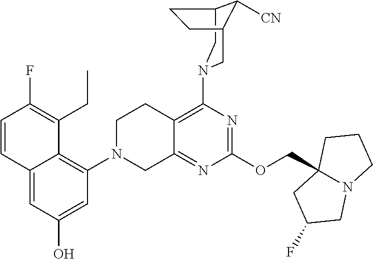 [SMILES: CCc1c(F)ccc2cc(O)cc(N3CCc4c(nc(OC[C@@]56CCCN5C[C@H](F)C6)nc4N4CC5CCC(C4)C5C#N)C3)c12]

### (1R,5S,8R)-3-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-3-azabicyclo[3.2.1]octane-8-carbonitrile

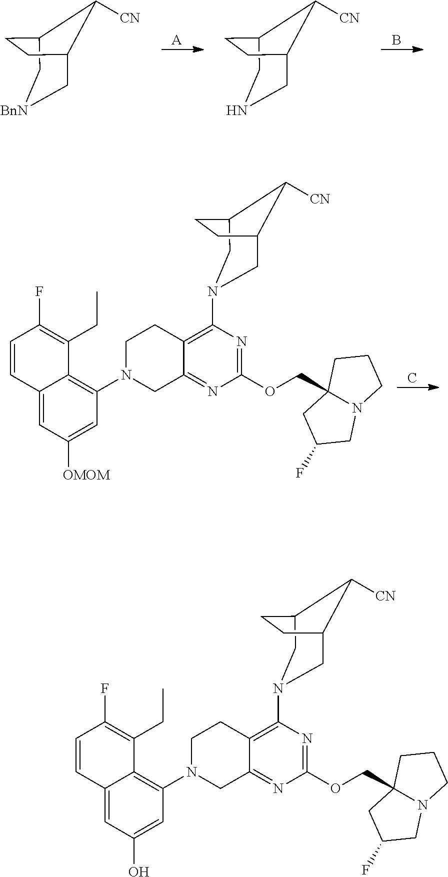

Step A. (1R,5S,8s)-3-azabicyclo[3.2.1]octane-8-carbonitrile: To a solution of (1R,5S,8s)-3-benzyl-3-azabicyclo[3.2.1]octane-8-carbonitrile (800 mg, 1.0 equiv) in MeOH (10.0 mL) was added Pd/C (80.0 mg, 10% purity) and AcOH (425 mg, 2.0 equiv) under N₂ atmosphere. The suspension was degassed and purged with H₂ for 3 times. The reaction was stirred at 25° C. for 3 hours under H₂ (15 Psi). The mixture was filtered and the filtrate was concentrated to afford the title compound (650 mg, crude, AcOH salt) as white solid; ¹H NMR (400 MHz, METHANOL-d₄) δ=3.20-3.05 (m, 4H), 2.87-2.74 (m, 1H), 2.65 (br s, 1H), 2.49-2.30 (m, 1H), 2.20-2.05 (m, 2H), 1.90-1.68 (m, 2H).

The last two steps were according to Example 248. The title compound was obtained as yellow solid. ¹H NMR (400 MHz, DMSO-d₆) δ=9.73-9.60 (m, 1H), 7.59 (dd, J=6.0, 9.0 Hz, 1H), 7.24 (t, J=9.6 Hz, 1H), 7.02-6.95 (m, 2H), 5.35-5.13 (m, 1H), 4.07 (br d, J=12.6 Hz, 1H), 3.96 (br d, J=8.0 Hz, 1H), 3.93 (s, 1H), 3.84 (dd, J=2.8, 10.4 Hz, 1H), 3.69-3.57 (m, 2H), 3.47-3.35 (m, 3H), 3.17-2.94 (m, 7H), 2.86-2.75 (m, 1H), 2.69-2.53 (m, 4H), 2.14-1.98 (m, 2H), 1.97-1.79 (m, 3H), 1.78-1.67 (m, 4H), 1.66-1.55 (m, 1H), 1.06 (t, J=7.2 Hz, 3H); LCMS (ESI, M+1): m/z=615.2.

### Example 653

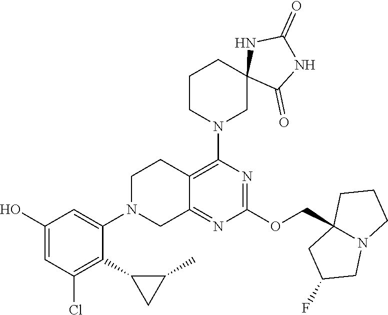 [SMILES: C[C@@H]1C[C@@H]1c1c(Cl)cc(O)cc1N1CCc2c(nc(OC[C@@]34CCCN3C[C@H](F)C4)nc2N2CCC[C@]3(C2)NC(=O)NC3=O)C1]

### (R)-7-(7-(3-chloro-5-hydroxy-2-(cis-2-methylcyclopropyl)phenyl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione

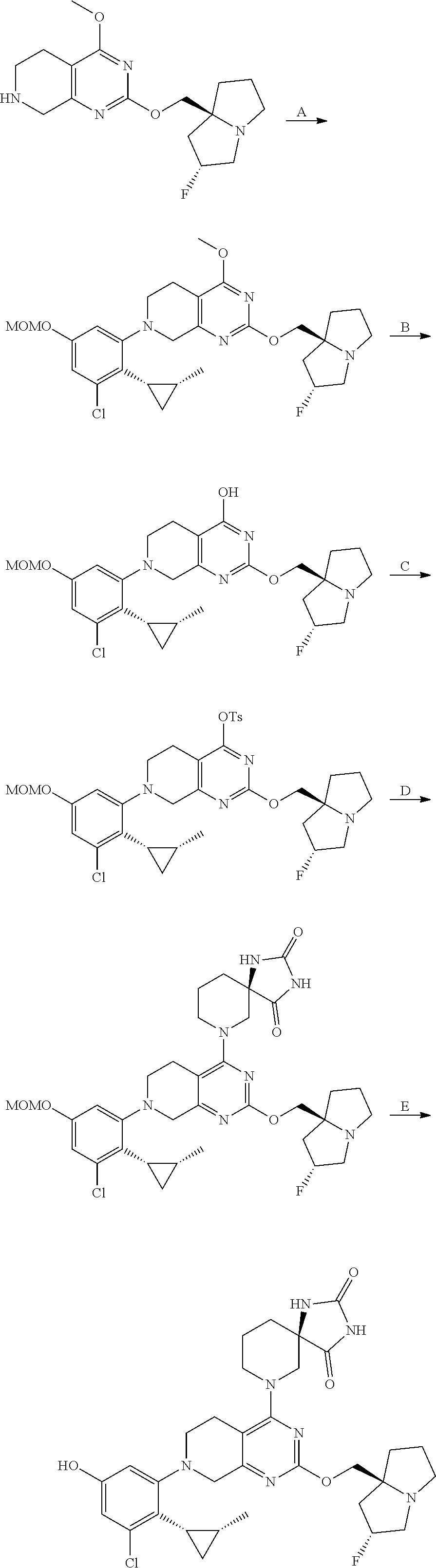

Step A. 7-(3-chloro-5-(methoxymethoxy)-2-(cis-2-methylcyclopropyl)phenyl)-2-(((2R,7aS)-2-fluorotetrahydro-1H-pyrrolizin-7a(5H)-yl)methoxy)-4-methoxy-5,6,7,8-tetrahydropyrido[3,4-d]pyrimidine: To a solution of 2-(((2R,7aS)-2-fluorotetrahydro-1H-pyrrolizin-7a(5H)-yl)methoxy)-4-methoxy-5,6,7,8-tetrahydropyrido[3,4-d]pyrimidine (1.00 g, 1.0 equiv) and 1-bromo-3-chloro-5-(methoxymethoxy)-2-(cis-2-methylcyclopropyl)benzene (758 mg, 0.8 equiv) in dioxane (8 mL) were added Xantphos Pd G4 (298 mg, 0.1 equiv) and Cs₂CO₃ (3.03 g, 3.0 equiv). The reaction was degassed and purged with nitrogen for 3 times. The reaction was stirred at 90° C. for 16 hours under nitrogen atmosphere. The mixture was diluted with water (20 mL) and extracted with ethyl acetate (2×20 mL). The combined organic layers were washed with brine (30 mL), dried over anhydrous sodium sulfate, concentrated and purified with column chromatography [SiO₂, petroleum ether/ethyl acetate=50/1 to 0/1] to afford the title compound (280 mg, 15% yield) as yellow solid; LCMS (ESI, M+1): m/z=547.1.

Step B. 7-(3-chloro-5-(methoxymethoxy)-2-(cis-2-methylcyclopropyl)phenyl)-2-(((2R,7aS)-2-fluorotetrahydro-1H-pyrrolizin-7a(5H)-yl)methoxy)-5,6,7,8-tetrahydropyrido[3,4-d] pyrimidin-4-ol: To a solution of 7-(3-chloro-5-(methoxymethoxy)-2-(cis-2-methylcyclopropyl)phenyl)-2-(((2R,7aS)-2-fluorotetrahydro-1H-pyrrolizin-7a(5H)-yl)methoxy)-4-methoxy-5,6,7,8-tetrahydropyrido[3,4-d]pyrimidine (320 mg, 1.0 equiv) in DMAc (4 mL) was added NaSEt (246 mg, 5.0 equiv). The reaction was stirred at 60° C. for 1 hour. The mixture was diluted with water (20 mL) and extracted with ethyl acetate (3×10 mL). The combined organic layers were washed with brine (30 mL), dried over anhydrous sodium sulfate, concentrated and purified with column chromatography [SiO₂, petroleum ether/ethyl acetate=50/1 to 0/1] to afford the title compound (170 mg, 44% yield) as yellow solid; LCMS (ESI, M+1): m/z=533.3.

Step C. 7-(3-chloro-5-(methoxymethoxy)-2-(cis-2-methylcyclopropyl)phenyl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate: To a solution of 7-(3-chloro-5-(methoxymethoxy)-2-(cis-2-methylcyclopropyl)phenyl)-2-(((2R,7aS)-2-fluorotetrahydro-1H-pyrrolizin-7a(5H)-yl)methoxy)-5,6,7,8-tetrahydropyrido[3,4-d]pyrimidin-4-ol (170 mg, 1.0 equiv), TEA (96.8 mg, 3.0 equiv) and DMAP (7.79 mg, 0.2 equiv) in DCM (10 mL) was slowly added 4-methylbenzenesulfonyl chloride (91.2 mg, 1.5 equiv) at 0° C. The reaction was stirred at 25° C. for 16 hours. The mixture was diluted with water (10 mL) and extracted with DCM (2×10 mL). The combined organic layers were washed with brine (20 mL), dried over anhydrous sodium sulfate, concentrated and purified with column chromatography [SiO₂, petroleum ether/ethyl acetate=50/1 to 0/1] to afford the title compound (177 mg, 77% yield) as yellow solid; LCMS (ESI, M+1): m/z=687.3.

Step D. (R)-7-(7-(3-chloro-5-(methoxymethoxy)-2-(cis-2-methylcyclopropyl)phenyl)-2-(((2R,7aS)-2-fluorotetrahydro-1H-pyrrolizin-7a(5H)-yl)methoxy)-5,6,7,8-tetrahydropyrido[3,4-d] pyrimidin-4-yl)-1,3,7-triazaspiro[4.5]decane-2,4-dione: To a solution of 7-(3-chloro-5-(methoxymethoxy)-2-(cis-2-methylcyclopropyl)phenyl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (90.0 mg, 1.0 equiv) and (R)-1,3,7-triazaspiro[4.5]decane-2,4-dione (33.2 mg, 1.5 equiv) in DMF (2 mL) were added DIEA (50.8 mg, 3.0 equiv) and 4 Å molecular sieve (20.0 mg). The reaction was stirred at 45° C. for 12 hours. The mixture was diluted with water (10 mL) and extracted with ethyl acetate (3×10 mL). The combined organic layers were washed with brine (20 mL), dried over anhydrous sodium sulfate, concentrated and purified with prep-TLC [SiO₂, DCM/MeOH=10/1] to afford the title compound (48.0 mg, 53% yield) as yellow solid; LCMS (ESI, M+1): m/z=684.4.

Step E. (R)-7-(7-(3-chloro-5-hydroxy-2-(cis-2-methylcyclopropyl)phenyl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione: To a solution of (R)-7-(7-(3-chloro-5-(methoxymethoxy)-2-(cis-2-methylcyclopropyl)phenyl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (40.0 mg, 1.0 equiv) in MeCN (1.5 mL) was added HCl•dioxane (4M, 1 mL) at 0° C. The reaction was stirred at 0° C. for 0.5 hours. The mixture was concentrated, dissolved in MeOH (2 mL), adjusted to pH=7 with solid NaHCO₃ and filtered. The filtrate was concentrated and purified with prep-HPLC [Waters Xbridge 150×25 mm×5 μm; mobile phase: water (NH₄HCO₃)-ACN; gradient: 40%-70% B over 9 min] to afford the title compound (20.9 mg, 55% yield) as white solid; ¹H NMR (400 MHz, DMSO-d₆) δ=10.76 (br d, J=4.4 Hz, 1H), 9.60 (s, 1H), 8.55 (br d, J=3.6 Hz, 1H), 6.53 (d, J=2.0 Hz, 1H), 6.44-6.36 (m, 1H), 5.35-5.14 (m, 1H), 3.97-3.78 (m, 5H), 3.21-2.69 (m, 10H), 2.11-1.66 (m, 12H), 1.30-1.19 (m, 1H), 1.18-0.96 (m, 1H), 0.79-0.59 (m, 4H); LCMS (ESI, M+1): m/z=640.4.

### Example 654

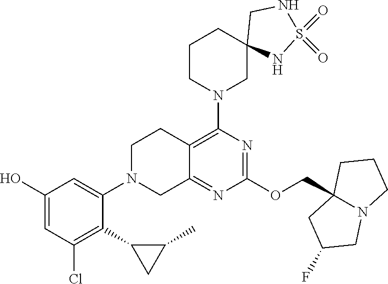 [SMILES: C[C@@H]1C[C@@H]1c1c(Cl)cc(O)cc1N1CCc2c(nc(OC[C@@]34CCCN3C[C@H](F)C4)nc2N2CCC[C@]3(CNS(=O)(=O)N3)C2)C1]

### (R)-7-(7-(3-chloro-5-hydroxy-2-(cis-2-methylcyclopropyl)phenyl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-2-thia-1,3,7-triazaspiro[4.5]decane 2,2-dioxide

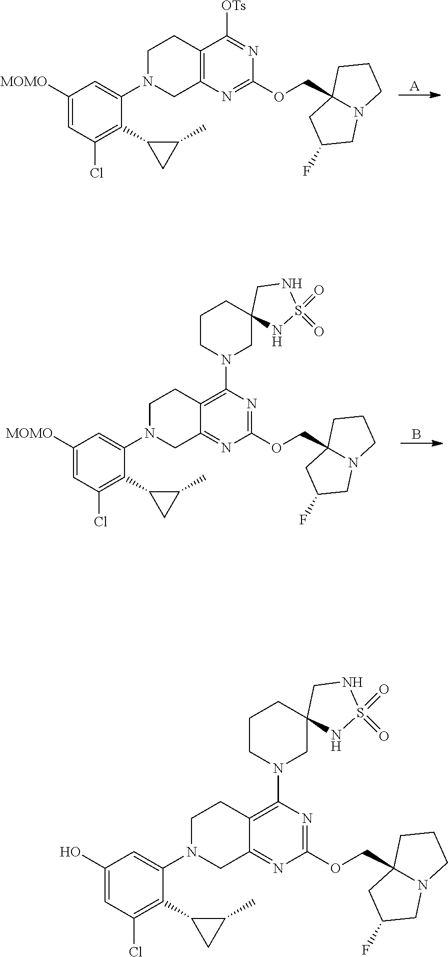

Step A. (R)-7-(7-(3-chloro-5-(methoxymethoxy)-2-(cis-2-methylcyclopropyl)phenyl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-2-thia-1,3,7-triazaspiro[4.5]decane 2,2-dioxide: To a solution of 7-(3-chloro-5-(methoxymethoxy)-2-(cis-2-methylcyclopropyl)phenyl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (80.0 mg, 1.0 equiv) and (R)-2-thia-1,3,7-triazaspiro[4.5]decane 2,2-dioxide (33.4 mg, 1.5 equiv) in DMF (2 mL) were added DIEA (45.1 mg, 3.0 equiv) and 4 Å molecular sieve (20.0 mg). The reaction was stirred at 45° C. for 12 hours. The mixture was diluted with water (10 mL) and extracted with ethyl acetate (3×10 mL). The combined organic layers were washed with brine (20 mL), dried over anhydrous sodium sulfate, concentrated and purified with prep-TLC [SiO₂, DCM/MeOH=10/1] to afford the title compound (54.0 mg, 55% yield) as yellow solid; LCMS (ESI, M+1): m/z=706.6.

Step B. (R)-7-(7-(3-chloro-5-hydroxy-2-(cis-2-methylcyclopropyl)phenyll)-2-(((2R,7aS)-2-fluorotetrahydro-1H-pyrrolizin-7a(5H)-yl)methoxy)-5,6,7,8-tetrahydropyrido[3,4-d] pyrimidin-4-yl)-2-thia-1,3,7-triazaspiro[4.5]decane 2,2-dioxide: To a solution of (R)-7-(7-(3-chloro-5-(methoxymethoxy)-2-(cis-2-methylcyclopropyl)phenyl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-2-thia-1,3,7-triazaspiro[4.5]decane 2,2-dioxide (55.0 mg, 1 equiv) in MeOH (5 mL) was added HCl•MeOH (4M, 1 mL) at 0° C. The reaction was stirred at 20° C. for 2 hours. The mixture was concentrated, dissolved in MeOH (2 mL), adjusted to pH=7 with solid NaHCO₃ and filtered. The filtrate was concentrated and purified with prep-HPLC [Waters Xbridge 150×25 mm×5 μm; mobile phase: water (NH₄HCO₃)-ACN; gradient: 48%-78% B over 9 min] to afford the title compound (20.9 mg, 55% yield) as white solid; ¹H NMR (400 MHz, DMSO-d₆) δ=9.58 (br s, 1H), 7.20-7.03 (m, 2H), 6.53 (d, J=2.0 Hz, 1H), 6.39 (dd, J=2.4, 4.4 Hz, 1H), 5.26 (br d, J=54.0 Hz, 1H), 4.30-4.03 (m, 1H), 3.99-3.91 (m, 1H), 3.86-3.79 (m, 2H), 3.57-3.38 (m, 2H), 3.26-2.95 (m, 8H), 2.89-2.67 (m, 3H), 2.15-1.95 (m, 3H), 1.94-1.56 (m, 9H), 1.33-1.21 (m, 1H), 1.10 (br s, 1H), 0.73 (br s, 1H), 0.65 (br d, J=4.8 Hz, 3H); LCMS (ESI, M+1): m/z=662.4.

### Example 655

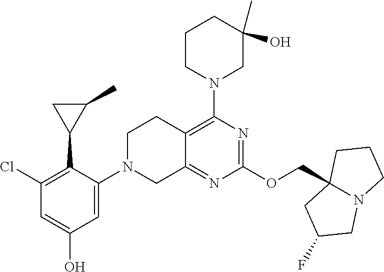 [SMILES: C[C@@H]1C[C@@H]1c1c(Cl)cc(O)cc1N1CCc2c(nc(OC[C@@]34CCCN3C[C@H](F)C4)nc2N2CCC[C@@](C)(O)C2)C1]

### (R)-1-(7-(3-chloro-5-hydroxy-2-(cis-2-methylcyclopropyl)phenyl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-3-methylpiperidin-3-ol

Synthesized according to Example 654. The title compound was obtained as white solid. ¹H NMR (400 MHz, METHANOL-d₄) δ=6.58 (d, J=2.0 Hz, 1H), 6.43 (d, J=2.0 Hz, 1H), 5.39-5.23 (m, 1H), 4.27-4.02 (m, 3H), 3.84-3.74 (m, 1H), 3.69-3.40 (m, 3H), 3.31-2.98 (m, 8H), 2.95-2.80 (m, 2H), 2.27-1.73 (m, 1OH), 1.35-1.30 (m, 1H), 1.24 (d, J=14.0 Hz, 3H), 1.17-1.09 (m, 1H), 0.92-0.82 (m, 1H), 0.73 (d, J=6.0 Hz, 3H); LCMS (ESI, M+1): m/z=586.5.

### Example 656

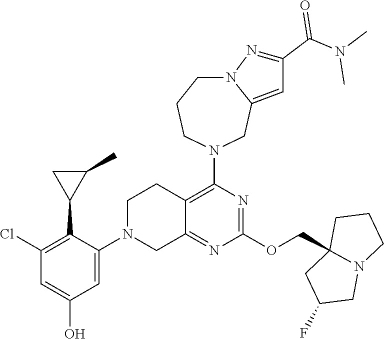 [SMILES: C[C@@H]1C[C@@H]1c1c(Cl)cc(O)cc1N1CCc2c(nc(OC[C@@]34CCCN3C[C@H](F)C4)nc2N2CCCn3nc(C(=O)N(C)C)cc3C2)C1]

### 5-(7-(3-chloro-5-hydroxy-2-(cis-2-methylcyclopropyl)phenyl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide

Synthesized according to Example 654. The title compound was obtained as white solid. ¹H NMR (400 MHz, METHANOL-d₄) δ=6.58 (d, J=2.4 Hz, 2H), 6.40 (d, J=2.0 Hz, 1H), 5.37-5.22 (m, 1H), 4.98-4.90 (m, 2H), 4.84 (br d, J=1.6 Hz, 1H), 4.53 (br t, J=4.8 Hz, 2H), 4.36-4.17 (m, 1H), 4.17-3.92 (m, 5H), 3.91-3.76 (m, 1H), 3.32 (br s, 3H), 3.28-3.15 (m, 4H), 3.08 (s, 3H), 3.04-3.00 (m, 1H), 2.97-2.82 (m, 2H), 2.26-1.86 (m, 8H), 1.37-1.31 (m, 1H), 1.22-1.07 (m, 1H), 0.91-0.80 (m, 1H), 0.74 (d, J=6.0 Hz, 3H); LCMS (ESI, M+1): m/z=679.4.

### Example 657

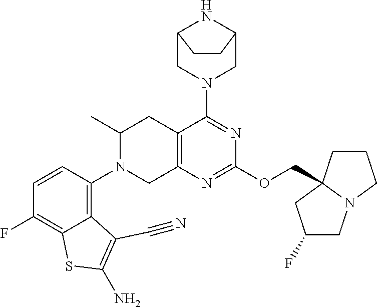 [SMILES: CC1Cc2c(nc(OC[C@@]34CCCN3C[C@H](F)C4)nc2N2CC3CCC(C2)N3)CN1c1ccc(F)c2sc(N)c(C#N)c12]

### 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-6-methyl-5,8-dihydropyrido[3,4-d]pyrimidin-7(6H)-yl)-2-amino-7-fluorobenzo[b]thiophene-3-carbonitrile

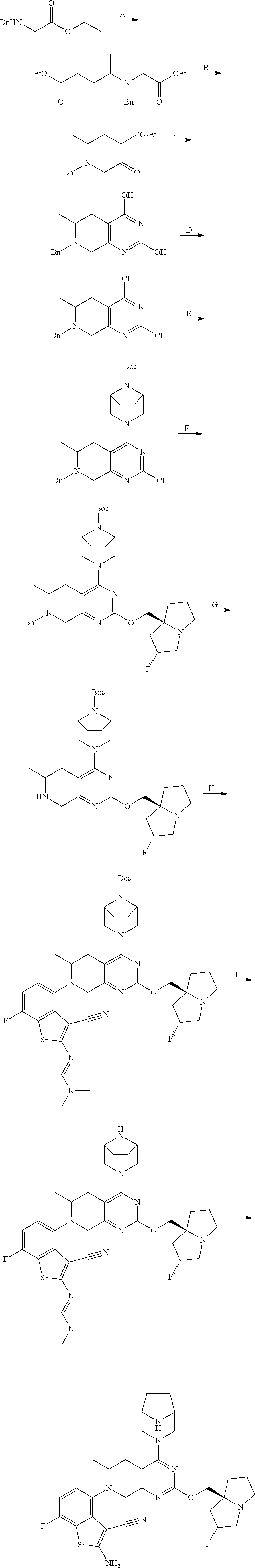

Step A. ethyl 4-(benzyl(2-ethoxy-2-oxoethyl)amino)pentanoate: To a solution of ethyl benzylglycinate (80.0 g, 1.0 equiv) and ethyl 4-oxopentanoate (89.5 g, 1.5 equiv) in DCM (2 L) were added NaBH(OAc)₃ (263 g, 3.0 equiv) and HOAc (24.9 g, 1.0 equiv). The reaction was stirred at 25° C. for 16 hours. The mixture was diluted with water (500 mL) and extracted with DCM (2×500 mL). The combined organic layers were washed with brine (500 mL), dried over anhydrous sodium sulfate, concentrated and purified by prep-HPLC [column: Phenomenex luna c18 250 mm×100 mm×10 μm; mobile phase: water(NH₄HCO₃)-ACN];gradient:60%-90% B over 20 min] to afford the title compound (30.0 g, 95% purity) as yellow oil; LCMS (ESI, M+1): m/z=322.2.

Step B. ethyl 1-benzyl-2-methyl-5-oxopiperidine-4-carboxylate: To a solution of ethyl 4-[benzyl-(2-ethoxy-2-oxo-ethyl)amino]pentanoate (20.0 g, 1.0 equiv) in toluene (250 mL) was added tBuOK (1 M, 81 mL, 1.3 equiv). The reaction was stirred at 25° C. for 3 hours. The mixture was diluted with water (200 mL) and extracted with DCM (2×200 mL). The combined organic layers were washed with brine (200 mL), dried over anhydrous sodium sulfate, concentrated and purified by column chromatography [SiO₂, petroleum ether/ethyl acetate=I/O to 5/1] to afford the title compound (6.00 g, 86% purity) as yellow oil; LCMS (ESI, M+1): m/z=276.2.

Step C. 7-benzyl-6-methyl-5,6,7,8-tetrahydropyrido[3,4-d]pyrimidine-2,4-diol: Na (3.00 g, 6.0 equiv) was added in portions to EtOH (80 mL) under nitrogen atmosphere at 20° C. and the reaction was stirred at 20° C. for 0.5 hours under nitrogen atmosphere. Then urea (2.20 g, 1.7 equiv) and ethyl 1-benzyl-2-methyl-5-oxo-piperidine-4-carboxylate (6.00 g, 1.0 equiv) were added to the reaction. The reaction was stirred at 85° C. for 12 hours. The mixture was diluted with water (50 mL) and extracted with ethyl acetate (3×50 mL). The combined organic layers were washed with brine (50 mL), dried over anhydrous sodium sulfate, concentrated and purified with prep-HPLC [0.1% FA condition] to afford the title compound (1.30 g, 85% purity) as yellow solid; LCMS (ESI, M+1): m/z=272.3.

Step D. 7-benzyl-2,4-dichloro-6-methyl-5,6,7,8-tetrahydropyrido[3,4-d] pyrimidine: A solution of 7-benzyl-6-methyl-6,8-dihydro-5H-pyrido[3,4-d]pyrimidine-2,4-diol (1.20 g, 1.0 equiv) in POC3 (15 mL) was stirred at 110° C. for 2 hours. The mixture was quenched with water (100 mL) and extracted with ethyl acetate (3×100 mL). The organic layers were washed with brine (150 mL), dried over anhydrous sodium sulfate and concentrated to afford the title compound (850 mg, crude) as yellow solid; LCMS (ESI, M+1): m/z=308.1.

Step E. tert-butyl (1R,5S)-3-(7-benzyl-2-chloro-6-methyl-5,6,7,8-tetrahydropyrido[3,4-d]pyrimidin-4-yl)-3,8-diazabicyclo[3.2.1]octane-8-carboxylate: To a solution of 7-benzyl-2,4-dichloro-6-methyl-6,8-dihydro-5H-pyrido[3,4-d]pyrimidine (800 mg, 1.0 equiv) and tert-butyl 3,8-diazabicyclo[3.2.1]octane-8-carboxylate (827 mg, 1.5 equiv) in DMF (10 mL) was added DIEA (1.00 g, 3.0 equiv). The reaction was stirred at 80° C. for 2 hours. The mixture was diluted with water (50 mL) and extracted with ethyl acetate (3×50 mL). The combined organic layers were washed with brine (100 mL), dried over anhydrous sodium sulfate, concentrated and purified with prep-HPLC [column: Waters Xbridge 150×25 mm×5 μm; mobile phase: water (ammonia hydroxide v/v)-ACN; gradient: 60%-90% B over min] to afford the title compound (600 mg, 98% purity) as white solid; LCMS (ESI, M+1): m/z=484.3.

Step F. tert-butyl (1R,5S)-3-(7-benzyl-2-(((2R,7aS)-2-fluorotetrahydro-1H-pyrrolizin-7a(5H)-yl)methoxy)-6-methyl-5,6,7,8-tetrahydropyrido[3,4-d] pyrimidin-4-yl)-3,8-diazabicyclo[3.2.1]octane-8-carboxylate: To a solution of tert-butyl 3-(7-benzyl-2-chloro-6-methyl-6,8-dihydro-5H-pyrido[3,4-d]pyrimidin-4-yl)-3,8-diazabicyclo[3.2.1]octane-8-carboxylate (600 mg, 1.0 equiv) and [(2R,8S)-2-fluoro-1,2,3,5,6,7-hexahydropyrrolizin-8-yl]methanol (395 mg, 2.0 equiv) in toluene (10 mL) were added BINAP (154 mg, 0.2 equiv), Pd(OAc)₂ (27.8 mg, 0.1 equiv) and Cs₂CO₃ (808 mg, 2.0 equiv). The reaction was degassed and purged with nitrogen for 3 times. The reaction was stirred at 110° C. for 5 hours under nitrogen atmosphere. The mixture was diluted with water (10 mL) and extracted with ethyl acetate (3×10 mL). The combined organic layers were washed with brine (20 mL), dried over anhydrous sodium sulfate, concentrated and purified with prep-HPLC [column: Waters Xbridge 150×25 mm x Sum; mobile phase: water (ammonia hydroxide v/v)-ACN; gradient:65%-95% B over min] to afford the title compound (430 mg, 95% purity) as white solid; LCMS (ESI, M+1): m/z=607.4.

Step G. tert-butyl (1R,5S)-3-(2-(((2R,7aS)-2-fluorotetrahydro-1H-pyrrolizin-7a(5H)-yl)methoxy)-6-methyl-5,6,7,8-tetrahydropyrido[3,4-d] pyrimidin-4-yl)-3,8-diazabicyclo[3.2.1]octane-8-carboxylate: To a solution of tert-butyl (1R,5S)-3-(7-benzyl-2-(((2R,7aS)-2-fluorotetrahydro-1H-pyrrolizin-7a(5H)-yl)methoxy)-6-methyl-5,6,7,8-tetrahydropyrido[3,4-d]pyrimidin-4-yl)-3,8-diazabicyclo[3.2.1]octane-8-carboxylate (400 mg, 1.0 equiv) in EtOH (10 mL) were added Pd(OH)₂ (150 mg) and Pd/C (150 mg). The reaction was degassed and purged with hydrogen for 3 times. Then the reaction was stirred at 25° C. for 1 hour under hydrogen atmosphere. The mixture was filtered, concentrated and purified with prep-HPLC [column: Waters Xbridge 150×25 mm×5 μm; mobile phase: water (NH₃•H₂O)-ACN; gradient: 38%-68% B over 10 min] to afford the title compound (240 mg, 98.7% purity) as white solid; LCMS (ESI, M+1): m/z=517.4.

Step H. tert-butyl (1R,5S)-3-(7-(3-cyano-2-(((E)-(dimethylamino)methylene)amino)-7-fluorobenzo[b]thiophen-4-yl)-2-(((2R,7aS)-2-fluorotetrahydro-1H-pyrrolizin-7a(5H)-yl)methoxy)-6-methyl-5,6,7,8-tetrahydropyrido[3,4-d]pyrimidin-4-yl)-3,8-diazabicyclo[3.2.1]octane-8-carboxylate: To a solution of tert-butyl (1R,5S)-3-(2-(((2R,7aS)-2-fluorotetrahydro-1H-pyrrolizin-7a(5H)-yl)methoxy)-6-methyl-5,6,7,8-tetrahydropyrido[3,4-d]pyrimidin-4-yl)-3,8-diazabicyclo[3.2.1]octane-8-carboxylate (200 mg, 1.0 equiv) and (E)-N′-(4-bromo-3-cyano-7-fluoro-benzothiophen-2-yl)-N,N-dimethyl-formamidine (152 mg, 1.2 equiv) in dioxane (4 mL) were added Pd-PEPPSI-IPentCl (37.7 mg, 0.1 equiv) and Cs₂CO₃ (378 mg, 3.0 equiv). The reaction was degassed and purged with nitrogen for 3 times. Then the reaction was stirred at 90° C. for 2 hours under nitrogen atmosphere. The mixture was diluted with water (10 mL) and extracted with ethyl acetate (3×10 mL). The combined organic layers were washed with brine (10 mL), dried over anhydrous sodium sulfate, concentrated and purified with prep-TLC (petroleum ether/ethyl acetate=0/1) to afford the title compound (90 mg, 60% purity) as yellow solid; LCMS (ESI, M+1): m/z=762.4.

Step I. (E)-N′-(4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-6-methyl-5,8-dihydropyrido[3,4-d]pyrimidin-7(6H)-yl)-3-cyano-7-fluorobenzo[b]thiophen-2-yl)-N,N-dimethylformimidamide: To a solution of tert-butyl (1R,5S)-3-(7-(3-cyano-2-(((E)-(dimethylamino)methylene)amino)-7-fluorobenzo[b]thiophen-4-yl)-2-(((2R,7aS)-2-fluorotetrahydro-1H-pyrrolizin-7a(5H)-yl)methoxy)-6-methyl-5,6,7,8-tetrahydropyrido[3,4-d]pyrimidin-4-yl)-3,8-diazabicyclo[3.2.1]octane-8-carboxylate (80.0 mg, 1.0 equiv) in MeOH (1 mL) was added HCl-MeOH (4M, 1 mL). The reaction was stirred at 25° C. for 1 hour. The mixture was concentrated to afford the title compound (80 mg, crude) as colorless oil; LCMS (ESI, M+1): m/z=662.3.

Step J. 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-6-methyl-5,8-dihydropyrido[3,4-d]pyrimidin-7(6H)-yl)-2-amino-7-fluorobenzo[b]thiophene-3-carbonitrile: To a solution of (E)-N′-(4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)-6-methyl-5,8-dihydropyrido[3,4-d]pyrimidin-7(6H)-yl)-3-cyano-7-fluorobenzo[b]thiophen-2-yl)-N,N-dimethylformimidamide(80.0 mg, 1.0 equiv) in DMAc (3 mL) was added K₃PO₄ (2 M, 3 mL). The reaction was stirred at 80° C. for 1 hour. The mixture was diluted with water (10 mL) and extracted with ethyl acetate (2×10 mL). The combined organic layers were washed with brine (10 mL), dried over anhydrous sodium sulfate, concentrated and purified with prep-HPLC [column: YMC-Actus Triart C18 150×30 mm×7 μm; mobile phase: water(FA)-ACN; gradient:13%-43% B over 10 min] to afford the title compound (15.2 mg, 97.5% purity, 0.94 HCOOH) as yellow solid; ¹H NMR (400 MHz, METHANOL-d₄) δ=7.20 (dd, J=4.8, 8.8 Hz, 1H), 6.90 (t, J=8.8 Hz, 1H), 5.43-5.22 (m, 1H), 4.38 (d, J=13.2 Hz, 1H), 4.25-4.17 (m, 1H), 4.16-4.10 (m, 1H), 4.06-3.82 (m, 5H), 3.60-3.51 (m, 1H), 3.42-3.32 (m, 2H), 3.29-3.20 (m, 2H), 3.10-3.04 (m, 1H), 2.99-2.88 (m, 1H), 2.64-2.52 (m, 1H), 2.41-2.09 (m, 5H), 2.08-1.98 (m, 4H), 1.95-1.89 (m, 2H), 1.06-0.90 (m, 3H); LCMS (ESI, M+1): m/z=607.3.

### Example 658

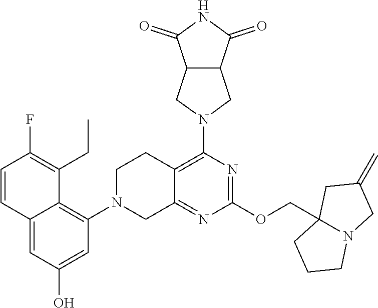 [SMILES: C=C1CN2CCCC2(COc2nc3c(c(N4CC5C(=O)NC(=O)C5C4)n2)CCN(c2cc(O)cc4ccc(F)c(CC)c24)C3)C1]

### 5-(7-(8-ethyl-7-fluoro-3-hydroxynaphthalen-1-yl)-2-((2-methylenetetrahydro-1H-pyrrolizin-7a(5H)-yl)methoxy)-5,6,7,8-tetrahydropyrido[3,4-d]pyrimidin-4-yl)tetrahydropyrrolo[3,4-c]pyrrole-1,3(2H,3aH)-dione

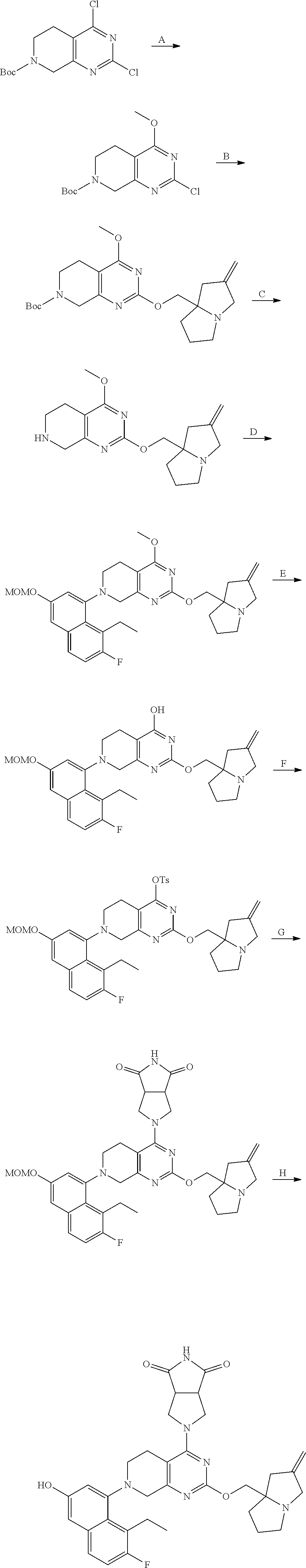

Step A. tert-butyl 2-chloro-4-methoxy-5,8-dihydropyrido[3,4-d]pyrimidine-7(6H)-carboxylate: To a solution of tert-butyl 2,4-dichloro-5,8-dihydropyrido[3,4-d]pyrimidine-7(6H) -carboxylate (20.0 g, 1.0 equiv) in MeOH (200 mL) was added NaOMe (23.7 g, 30% purity, 2.0 equiv) at 0° C. The reaction was stirred at 0° C. for 3 hours. The mixture was diluted with water (400 mL) and extracted with ethyl acetate (2×500 mL). The combined organic layers were washed with brine (300 mL), dried over anhydrous sodium sulfate, concentrated and purified with column chromatography [SiO₂, petroleum ether/ethyl acetate=50/1 to 10/1] to afford the title compound (14.5 g, 69% yield) as white solid; LCMS (ESI, M+1): m/z=300.1.

Step B. tert-butyl 4-methoxy-2-((2-methylenetetrahydro -1H-pyrrolizin -7a(5H)-yl)methoxy)-5,8-dihydropyrido[3,4-d]pyrimidine-7(6H)-carboxylate: To a solution of (2-methylenetetrahydro-1H-pyrrolizin-7a(5H)-yl)methanol (4.40 g, 1.0 equiv) and tert-butyl 2-chloro-4-methoxy-5,8-dihydropyrido[3,4-d]pyrimidine-7(6H)-carboxylate (8.60 g, 1.0 equiv) in toluene (100 mL) were added Pd(OAc)₂ (644 mg, 0.1 equiv), BINAP (3.57 g, 0.2 equiv) and Cs₂CO₃ (28.0 g, 3.0 equiv). The reaction was degassed and purged with N₂ for 3 times. The reaction was stirred at 110° C. for 2 hours under N₂ atmosphere. The mixture was diluted with water (200 mL) and extracted with ethyl acetate (2×300 mL). The combined organic layers were washed with brine (100 mL), dried over anhydrous sodium sulfate, concentrated and purified with column chromatography [SiO₂, petroleum ether/ethyl acetate=1/1 to 10/1] and prep-HPLC [0.1% FA condition] to afford the title compound (6.30 g, 53% yield) as yellow oil; LCMS (ESI, M+1): m/z=417.1.

Step C. 4-methoxy-2-((2-methylenetetrahydro-1H-pyrrolizin-7a(5H)-yl)methoxy)-5,6,7,8-tetrahydropyrido[3,4-d]pyrimidine: To a solution of tert-butyl 4-methoxy-2-((2-methylenetetrahydro-1H-pyrrolizin-7a(5H)-yl)methoxy)-5,8-dihydropyrido[3,4-d]pyrimidine-7(6H)-carboxylate (6.00 g, 1.0 equiv) in MeOH (30 mL) was added HCl-MeOH (4M, 30 mL). The reaction was stirred at 25° C. for 5 hours. The mixture was concentrated, diluted with DCM/MeOH=10/1 (60 mL) and washed with sodium hydroxide solution (1 M, 4×80 mL). The combined organic layers were washed with brine (40 mL), dried over anhydrous sodium sulfate and concentrated to afford the title compound (4.30 g, 94% yield) as yellow gum; LCMS (ESI, M+1): m/z=317.1.

Step D. 7-(8-ethyl-7-fluoro-3-(methoxymethoxy)naphthalen-1-yl)-4-methoxy-2-((2-methylenetetrahydro-1H-pyrrolizin-7a(5H)-yl)methoxy)-5,6,7,8-tetrahydropyrido[3,4-d] pyrimidine: To a solution of 4-methoxy-2-((2-methylenetetrahydro-1H-pyrrolizin-7a(5H)-yl) methoxy)-5,6,7,8-tetrahydropyrido[3,4-d]pyrimidine (3.30 g, 1.0 equiv) and 8-ethyl-7-fluoro-3-(methoxymethoxy)naphthalen-1-yl trifluoromethanesulfonate (4.79 g, 1.2 equiv) in toluene (55 mL) were added Pd₂(dba)₃ (955 mg, 0.1 equiv), Xantphos (1.21 g, 0.2 equiv) and Cs₂CO₃ (10.2 g, 3.0 equiv). The reaction was degassed and purged with N₂ for 3 times. The reaction was stirred at 110° C. for 12 hours under N₂ atmosphere. The mixture was diluted with water (80 mL) and extracted with ethyl acetate (2×100 mL). The combined organic layers were washed with brine (60 mL), dried over anhydrous sodium sulfate, concentrated and purified with column chromatography [SiO₂, petroleum ether:ethyl acetate=3:1 to 0:1; dichloromethane: methanol=10:1 to 0:1] and prep-HPLC [column: Phenomenex luna C18 (250×70 mm, 10 μm); mobile phase: water(FA)-ACN; gradient:30%-60% B over 20 min] to afford the title compound (1.15 g, 20% yield) as red solid; LCMS (ESI, M+1): m/z=549.3.

Step E. 7-(8-ethyl-7-fluoro-3-(methoxymethoxy)naphthalen-1-yl)-2-((2-methylenetetrahydro -1H-pyrrolizin-7a(5H)-yl)methoxy)-5,6,7,8-tetrahydropyrido[3,4-d]pyrimidin-4-ol: To a solution of 7-(8-ethyl-7-fluoro-3-(methoxymethoxy)naphthalen-1-yl)-4-methoxy-2-((2-methylenetetrahydro-1H-pyrrolizin-7a(5H)-yl)methoxy)-5,6,7,8-tetrahydropyrido[3,4-d]pyrimidine (1.14 g, 1.0 equiv) in DMAc (20 mL) was added NaSEt (874 mg, 5.0 equiv). The reaction was stirred at 60° C. for 0.5 hours. The mixture was diluted with water (100 mL) and extracted with ethyl acetate (3×80 mL). The combined organic layers were washed with brine (100 mL), dried over anhydrous sodium sulfate, concentrated and purified with prep-HPLC [column: Waters Xbridge 150×25 mm×5 μm; mobile phase: water ammonia hydroxide v/v-ACN; gradient:40%-70% B over min] to afford the title compound (784 mg, 67% yield) as yellow solid; LCMS (ESI, M+1): m/z=535.2.

Step F. 7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d] pyrimidin-4-yl 4-methylbenzenesulfonate: To a solution of 7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d]pyrimidin-4-ol (340 mg, 1.0 equiv) and TEA (193 mg, 3.0 equiv) in DCM (4.5 mL) were added 4-methylbenzenesulfonyl chloride (182 mg, 1.5 equiv) and DMAP (15.5 mg, 0.2 equiv) at 0° C. The reaction was stirred at 25° C. for 1 hour. The mixture was diluted with water (10 mL) and extracted with ethyl acetate (2×15 mL). The combined organic layers were washed with brine (10 mL), dried over anhydrous sodium sulfate, concentrated and purified with column chromatography [SiO₂, petroleum ether/ethyl acetate=0/1] to afford the title compound (374 mg, 81% yield) as yellow oil; LCMS (ESI, M+1): m/z=689.3.

Step G. 5-(7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d] pyrimidin-4-yl)tetrahydropyrrolo[3,4-c]pyrrole-1,3(2H,3aH)-dione: To a solution of 7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d]pyrimidin-4-yl 4-methylbenzenesulfonate (50.0 mg, 1.0 equiv) and DIEA (46.9 mg, 5.0 equiv) in DMF (2 mL) were added tetrahydropyrrolo [3,4-c]pyrrole-1,3(2H,3aH)-dione (20.3 mg, 2.0 equiv) and 4 Å molecular sieve (50.0 mg). The reaction was stirred at 80° C. for 12 hours. The mixture was diluted with water (10 mL) and extracted with ethyl acetate (3×10 mL). The combined organic layers were washed with brine (20 mL), dried over anhydrous sodium sulfate, concentrated and purified with prep-TLC [SiO2, dichloromethane: methanol=10:1] to afford the title compound (40.0 mg, 73% yield) as yellow solid; LCMS (ESI, M+1): m/z=657.3.

Step H. 5-(7-(8-ethyl-7-fluoro-3-hydroxynaphthalen-1-yl)-2-((2-methylenetetrahydro -1H-pyrrolizin-7a(5H)-yl)methoxy)-5,6,7,8-tetrahydropyrido[3,4-d]pyrimidin-4-yl)tetrahydropyrrolo[3,4-c]pyrrole-1,3(2H,3aH)-dione: To a solution of 5-(7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d]pyrimidin-4-yl)tetrahydropyrrolo[3,4-c]pyrrole-1,3(2H,3aH)-dione (40.0 mg, 1.0 equiv) in MeOH (1.5 mL) was added HCl-MeOH (4M, 1.5 mL) at 0° C. The reaction was stirred at 0° C. for 3 hours. The mixture was concentrated, dissolved in MeOH (2 mL), adjusted to pH=7 with solid NaHCO₃ and filtered. The filtrate was concentrated and purified with prep-HPLC [column: Waters Xbridge 150×25 mm×5 μm; mobile phase: water (NH₄HCO₃)-ACN; gradient:38%-68% B over 9 min] to afford the title compound (7.12 mg, 19% yield) as yellow solid; ¹H NMR (400 MHz, METHANOL-d₄) δ=7.53 (dd, J=5.6, 8.8 Hz, 1H), 7.16 (t, J=9.2 Hz, 1H), 7.00 (dd, J=2.4, 13.2 Hz, 2H), 5.00 (s, 2H), 4.66-4.54 (m, 1H), 4.27-4.10 (m, 3H), 4.05-4.01 (m, 1H), 3.87-3.63 (m, 3H), 3.60-3.46 (m, 4H), 3.43-3.34 (m, 3H), 3.26-3.11 (m, 3H), 2.80-2.66 (m, 3H), 2.50-2.43 (m, 1H), 2.19-2.07 (m, 1H), 2.01-1.76 (m, 3H), 1.12 (t, J=7.2 Hz, 3H); LCMS (ESI, M+1): m/z=613.3.

### Example 659

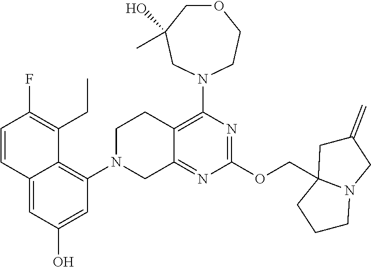 [SMILES: C=C1CN2CCCC2(COc2nc3c(c(N4CCOC[C@@](C)(O)C4)n2)CCN(c2cc(O)cc4ccc(F)c(CC)c24)C3)C1]

### (6S)-4-(7-(8-ethyl-7-fluoro-3-hydroxynaphthalen-1-yl)-2-((2-methylenetetrahydro-1H-pyrrolizin-7a(5H)-yl)methoxy)-5,6,7,8-tetrahydropyrido[3,4-d]pyrimidin-4-yl)-6-methyl-1,4-oxazepan-6-ol

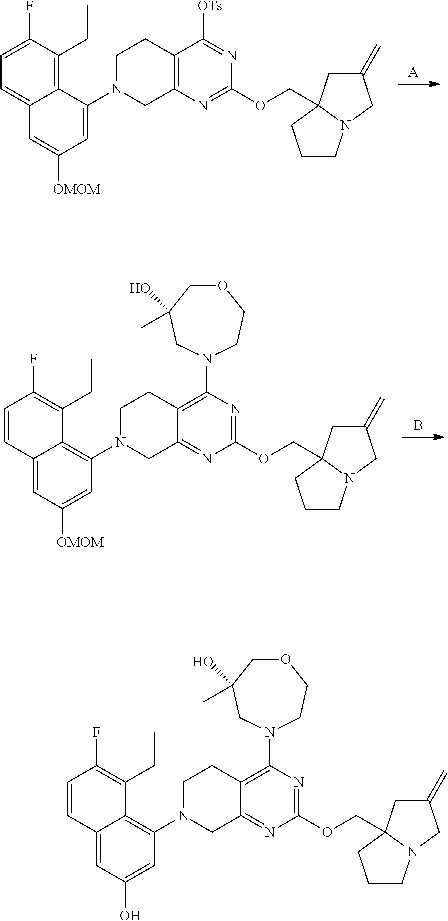

Step A. (6S)-4-(7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d]0pyrimidin-4-yl)-6-methyl-1,4-oxazepan-6-ol: To a solution of 7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d]pyrimidin-4-yl 4-methylbenzenesulfonate (90.0 mg, 1.0 equiv) and DIEA (84.4 mg, 5.0 equiv) in DMF (3 mL) were added (S)-6-methyl-1,4-oxazepan-6-ol (73.5 mg, 2.0 equiv, tartrate) and 4 Å molecular sieve (90.0 mg). The reaction was stirred at 80° C. for 12 hours. The mixture was diluted with water (10 mL) and extracted with ethyl acetate (3×10 mL). The combined organic layers were washed with brine (10 mL), dried over anhydrous sodium sulfate, concentrated and purified with prep-TLC [SiO₂, dichloromethane: methanol=10:1] to afford the title compound (64.0 mg, 60% yield) as yellow solid; LCMS (ESI, M+1): m/z=648.2.

Step B. (6S)-4-(7-(8-ethyl-7-fluoro-3-hydroxynaphthalen-1-yl)-2-((2-methylenetetrahydro-1H-pyrrolizin-7a(5H)-yl)methoxy)-5,6,7,8-tetrahydropyrido[3,4-d]0pyrimidin-4-yl)-6-methyl-1,4-oxazepan-6-ol: To a solution of (6S)-4-(7-(8-ethyl-7-fluoro -3-(methoxymethoxy)naphthalen-1-yl)-2-((2-methylenetetrahydro-1H-pyrrolizin -7a(5H)-yl) methoxy)-5,6,7,8-tetrahydropyrido[3,4-d]pyrimidin-4-yl)-6-methyl-1,4-oxazepan-6-ol (60.0 mg, 1.0 equiv) in MeOH (2 mL) was added HCl•MeOH (4M, 2 mL) at 0° C. The reaction was stirred at 0° C. for 4 hours. The mixture was concentrated, dissolved in MeOH (5 mL), adjusted to pH=7 with solid NaHCO₃ and filtered. The filtrate was concentrated and purified with prep-HPLC [column: Waters Xbridge 150×25 mm×5 μm; mobile phase: water(NH₄HCO₃)-ACN; gradient: 42%-72% B over 9 min] to afford the title compound (18.3 mg, 32% yield) as pink solid; ¹H NMR (400 MHz, METHANOL-d₄) δ=7.54-7.49 (m, 1H), 7.21-7.11 (m, 1H), 7.06-6.93 (m, 2H), 4.96 (s, 2H), 4.18-4.08 (m, 3H), 4.07-3.91 (m, 2H), 3.90-3.76 (m, 3H), 3.75-3.65 (m, 2H), 3.63-3.47 (m, 4H), 3.44-3.37 (m, 2H), 3.31-3.05 (m, 4H), 2.88-2.58 (m, 3H), 2.48-2.40 (m, 1H), 2.18-1.77 (m, 4H), 1.20 (s, 3H), 1.12 (t, J=7.2 Hz, 3H); LCMS (ESI, M+1): m/z=604.3.

### Example 660

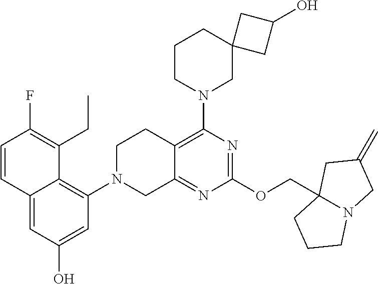 [SMILES: C=C1CN2CCCC2(COc2nc3c(c(N4CCCC5(CC(O)C5)C4)n2)CCN(c2cc(O)cc4ccc(F)c(CC)c24)C3)C1]

### 6-(7-(8-ethyl-7-fluoro-3-hydroxynaphthalen-1-yl)-2-((2-methylenetetrahydro-1H-pyrrolizin-7a(5H)-yl)methoxy)-5,6,7,8-tetrahydropyrido[3,4-d]pyrimidin-4-yl)-6-azaspiro[3.5]nonan-2-ol

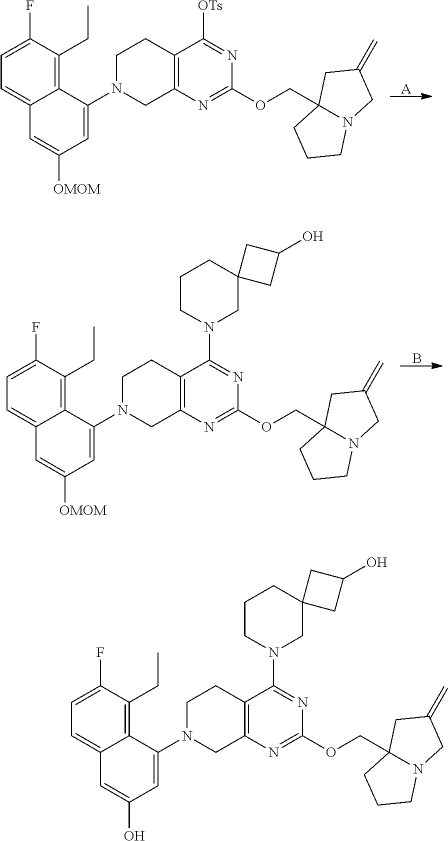

Synthesized according to Example 659. The title compound was obtained as yellow solid. ¹H NMR (400 MHz, METHANOL-d₄) δ=7.51 (dd, J=5.6, 8.8 Hz, 1H), 7.14 (t, J=9.2 Hz, 1H), 6.98 (dd, J=2.4, 12.0 Hz, 2H), 4.96 (s, 2H), 4.30-4.04 (m, 4H), 3.91-3.40 (m, 8H), 3.27-3.11 (m, 4H), 2.81-2.59 (m, 3H), 2.49-2.39 (m, 1H), 2.38-2.01 (m, 4H), 1.97-1.60 (m, 9H), 1.13 (td, J=2.4, 7.2 Hz, 3H); LCMS (ESI, M+1): m/z=614.4.

### Example 661

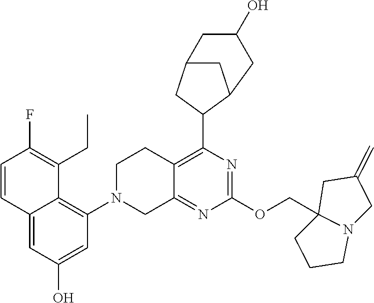 [SMILES: C=C1CN2CCCC2(COc2nc3c(c(C4CC5CC(O)CC4C5)n2)CCN(c2cc(O)cc4ccc(F)c(CC)c24)C3)C1]

### 6-(7-(8-ethyl-7-fluoro-3-hydroxynaphthalen-1-yl)-2-((2-methylenetetrahydro-1H-pyrrolizin-7a(5H)-yl)methoxy)-5,6,7,8-tetrahydropyrido[3,4-d]pyrimidin-4-yl)-6-azabicyclo[3.2.1]octan-3-ol

Synthesized according to Example 659. The title compound was obtained as off-white solid; ¹H NMVR (400 lVtVz, IVETHANOL-d₄) δ=7.51 (dd, J=6.0, 9.2 Hz, 1H), 7.14 (t, J=9.6 Hz, 1H), 7.01 (dd, J=2.4, 4.8 Hz, 1H), 6.96 (d, J 2.0 Hz, 1H), 4.97 (d, J 1.2 Hz, 2H), 4.66 (t, J=4.4 Hz, 1H), 4.18-4.07 (m, 2H), 4.04-3.92 (m, 2H), 3.81-3.75 (mn, 1H), 3.75-3.65 (mn, 3H), 3.61 (d, J=17.2 Hz, 1H), 3.53-3.47 (m, 1H), 3.44-3.37 (m, 2H), 3.22-3.07 (mn, 3H), 2.98 (d, J=13.6 Hz, 1H), 2.82-2.58 (m, 4H), 2.50-2.40 (m, 2H), 2.16-2.05 (m, 2H), 2.01-1.92 (m, 2H), 1.81 (dd, J=7.2, 12.4 Hz, 1H), 1.74 (d, J=11.6 Hz, 1H), 1.58-1.48 (m, 1H), 1.46-1.36 (m, 1H), 1.12 (q, J=7.2 Hz, 3H); LCMS (ESI, M+l): in/z=600.4. Example 662 to 708 were synthesized according to Example 514.

| Example No. | Observed M + 1 |
|---|---|
| 662 | 527.4 |
| 663 | 544.2 |
| 664 | 530.3 |
| 665 | 539.4 |
| 666 | 532.4 |
| 667 | 530.3 |
| 668 | 541.2 |
| 669 | 516.3 |
| 670 | 572.4 |
| 671 | 543.2 |
| 672 | 544.2 |
| 673 | 555.4 |
| 674 | 571.2 |
| 675 | 553.4 |
| 676 | 544.2 |
| 677 | 553.4 |
| 678 | 546.4 |
| 679 | 567.4 |
| 680 | 566.2 |
| 681 | 567.4 |
| 682 | 566.4 |
| 683 | 576.4 |
| 684 | 597.4 |
| 685 | 566.3 |
| 686 | 581.4 |
| 687 | 594.4 |
| 688 | 583.4 |
| 689 | 580.2 |
| 690 | 583.2 |
| 691 | 583.4 |
| 692 | 580.2 |
| 693 | 583.4 |
| 694 | 582.4 |
| 695 | 579.2 |
| 696 | 584.2 |
| 697 | 581.3 |
| 698 | 590.4 |
| 699 | 618.4 |
| 700 | 608.4 |
| 701 | 580.4 |
| 702 | 610.4 |
| 703 | 553.4 |
| 704 | 557.4 |
| 705 | 626.2 |
| 706 | 564.2 |
| 707 | 571.4 |
| 708 | 557.4 |

### Example 662

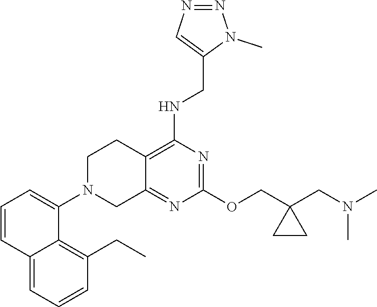 [SMILES: CCc1cccc2cccc(N3CCc4c(nc(OCC5(CN(C)C)CC5)nc4NCc4cnnn4C)C3)c12]

### 2-((1-((dimethylamino)methyl)cyclopropyl)methoxy)-7-(8-ethylnaphthalen-1-yl)-N-((1-methyl-1H-1,2,3-triazol-5-yl)methyl)-5,6,7,8-tetrahydropyrido[3,4-d]pyrimidin-4-amine

### Example 663

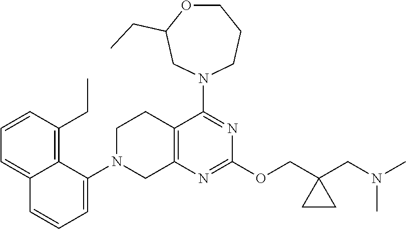 [SMILES: CCc1cccc2cccc(N3CCc4c(nc(OCC5(CN(C)C)CC5)nc4N4CCCOC(CC)C4)C3)c12]

### 1-(1-(((4-(2-ethyl-1,4-oxazepan-4-yl)-7-(8-ethylnaphthalen-1-yl)-5,6,7,8-tetrahydropyrido[3,4-d]pyrimidin-2-yl)oxy)methyl)cyclopropyl)-N,N-dimethylmethanamine

### Example 664

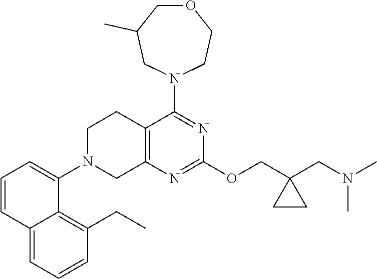 [SMILES: CCc1cccc2cccc(N3CCc4c(nc(OCC5(CN(C)C)CC5)nc4N4CCOCC(C)C4)C3)c12]

### 1-(1-(((7-(8-ethylnaphthalen-1-yl)-4-(6-methyl-1,4-oxazepan-4-yl)-5,6,7,8-tetrahydropyrido[3,4-d]pyrimidin-2-yl)oxy)methyl)cyclopropyl)-N,N-dimethylmethanamine

### Example 665

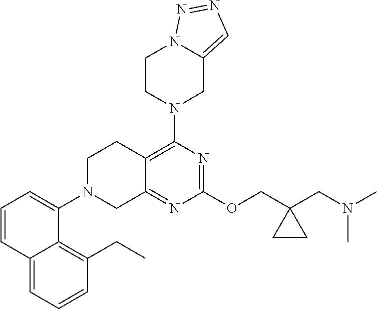 [SMILES: CCc1cccc2cccc(N3CCc4c(nc(OCC5(CN(C)C)CC5)nc4N4CCn5nncc5C4)C3)c12]

### 1-(1-(((4-(6,7-dihydro-[1,2,3]triazolo[1,5-a]pyrazin-5(4H)-yl)-7-(8-ethylnaphthalen-1-yl)-5,6,7,8-tetrahydropyrido[3,4-d]pyrimidin-2-yl)oxy)methyl)cyclopropyl)-N,N-dimethylmethanamine

### Example 666

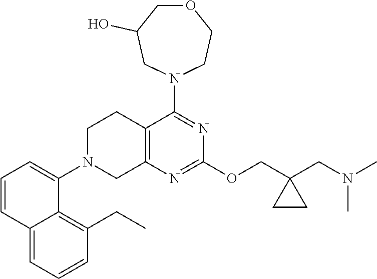 [SMILES: CCc1cccc2cccc(N3CCc4c(nc(OCC5(CN(C)C)CC5)nc4N4CCOCC(O)C4)C3)c12]

### 4-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)-1,4-oxazepan-6-ol

### Example 667

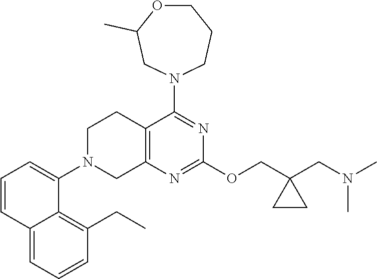 [SMILES: CCc1cccc2cccc(N3CCc4c(nc(OCC5(CN(C)C)CC5)nc4N4CCCOC(C)C4)C3)c12]

### 1-(1-(((7-(8-ethylnaphthalen-1-yl)-4-(2-methyl-1,4-oxazepan-4-yl)-5,6,7,8-tetrahydropyrido[3,4-d]pyrimidin-2-yl)oxy)methyl)cyclopropyl)-N,N-dimethylmethanamine

### Example 668

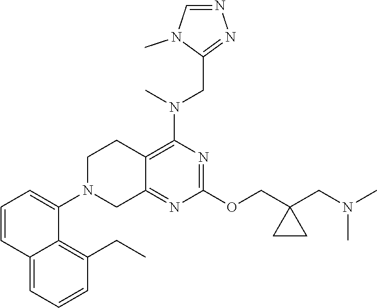 [SMILES: CCc1cccc2cccc(N3CCc4c(nc(OCC5(CN(C)C)CC5)nc4N(C)Cc4nncn4C)C3)c12]

### 2-((1-((dimethylamino)methyl)cyclopropyl)methoxy)-7-(8-ethylnaphthalen-1-yl)-N-methyl-N-((4-methyl-4H-1,2,4-triazol-3-yl)methyl)-5,6,7,8-tetrahydropyrido[3,4-d]pyrimidin-4-amine

### Example 669

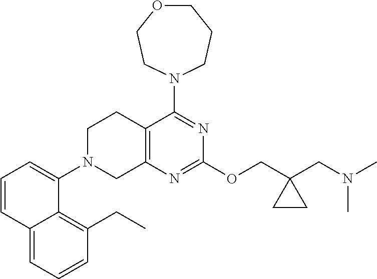 [SMILES: CCc1cccc2cccc(N3CCc4c(nc(OCC5(CN(C)C)CC5)nc4N4CCCOCC4)C3)c12]

### 1-(1-(((7-(8-ethylnaphthalen-1-yl)-4-(1,4-oxazepan-4-yl)-5,6,7,8-tetrahydropyrido[3,4-d]pyrimidin-2-yl)oxy)methyl)cyclopropyl)-N,N-dimethylmethanamine

### Example 670

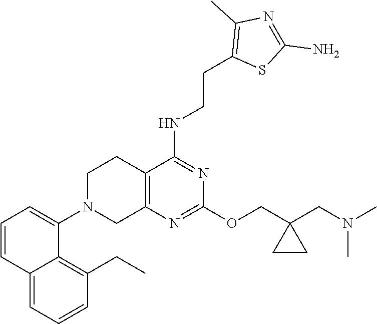 [SMILES: CCc1cccc2cccc(N3CCc4c(nc(OCC5(CN(C)C)CC5)nc4NCCc4sc(N)nc4C)C3)c12]

### 5-(2-((2-((1-((dimethylamino)methyl)cyclopropyl)methoxy)-7-(8-ethylnaphthalen-1-yl)-5,6,7,8-tetrahydropyrido[3,4-d]pyrimidin-4-yl)amino)ethyl)-4-methylthiazol-2-amine

### Example 671

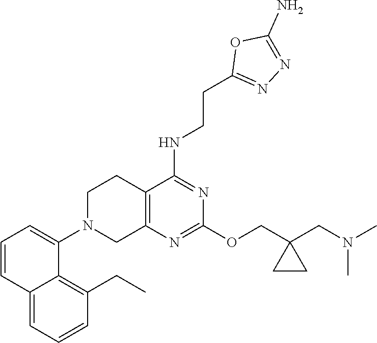 [SMILES: CCc1cccc2cccc(N3CCc4c(nc(OCC5(CN(C)C)CC5)nc4NCCc4nnc(N)o4)C3)c12]

### 5-(2-((2-((1-((dimethylamino)methyl)cyclopropyl)methoxy)-7-(8-ethylnaphthalen-1-yl)-5,6,7,8-tetrahydropyrido[3,4-d]pyrimidin-4-yl)amino)ethyl)-1,3,4-oxadiazol-2-amine

### Example 672

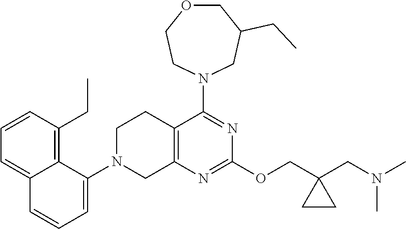 [SMILES: CCc1cccc2cccc(N3CCc4c(nc(OCC5(CN(C)C)CC5)nc4N4CCOCC(CC)C4)C3)c12]

### 1-(1-(((4-(6-ethyl-1,4-oxazepan-4-yl)-7-(8-ethylnaphthalen-1-yl)-5,6,7,8-tetrahydropyrido[3,4-d]pyrimidin-2-yl)oxy)methyl)cyclopropyl)-N,N-dimethylmethanamine

### Example 673

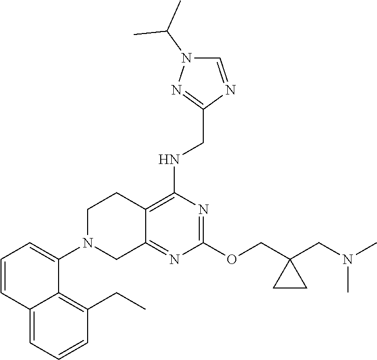 [SMILES: CCc1cccc2cccc(N3CCc4c(nc(OCC5(CN(C)C)CC5)nc4NCc4ncn(C(C)C)n4)C3)c12]

### 2-((1-((dimethylamino)methyl)cyclopropyl)methoxy)-7-(8-ethylnaphthalen-1-yl)-N-((1-isopropyl-1H-1,2,4-triazol-3-yl)methyl)-5,6,7,8-tetrahydropyrido[3,4-d]pyrimidin-4-amine

### Example 674

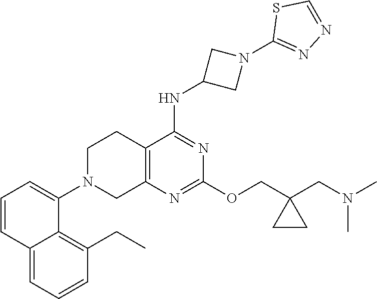 [SMILES: CCc1cccc2cccc(N3CCc4c(nc(OCC5(CN(C)C)CC5)nc4NC4CN(c5nncs5)C4)C3)c12]

### N-(1-(1,3,4-thiadiazol-2-yl)azetidin-3-yl)-2-((1-((dimethylamino)methyl)cyclopropyl)methoxy)-7-(8-ethylnaphthalen-1-yl)-5,6,7,8-tetrahydropyrido[3,4-d]pyrimidin-4-amine

### Example 675

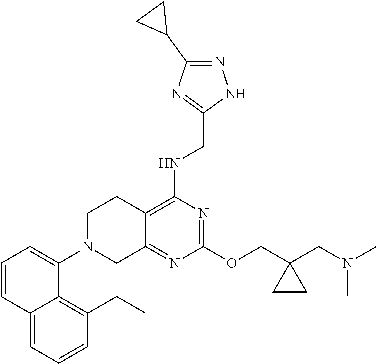 [SMILES: CCc1cccc2cccc(N3CCc4c(nc(OCC5(CN(C)C)CC5)nc4NCc4nc(C5CC5)n[nH]4)C3)c12]

### N-((3-cyclopropyl-1H-1,2,4-triazol-5-yl)methyl)-2-((1-((dimethylamino)methyl)cyclopropyl)methoxy)-7-(8-ethylnaphthalen-1-yl)-5,6,7,8-tetrahydropyrido[3,4-d]pyrimidin-4-amine

### Example 676

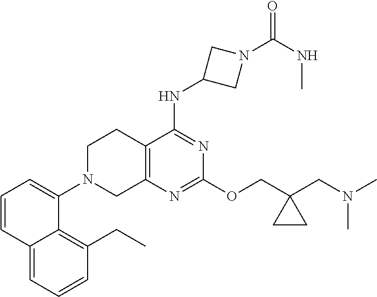 [SMILES: CCc1cccc2cccc(N3CCc4c(nc(OCC5(CN(C)C)CC5)nc4NC4CN(C(=O)NC)C4)C3)c12]

### 3-((2-((1-((dimethylamino)methyl)cyclopropyl)methoxy)-7-(8-ethylnaphthalen-1-yl)-5,6,7,8-tetrahydropyrido[3,4-d]pyrimidin-4-yl)amino)-N-methylazetidine-1-carboxamide

### Example 677

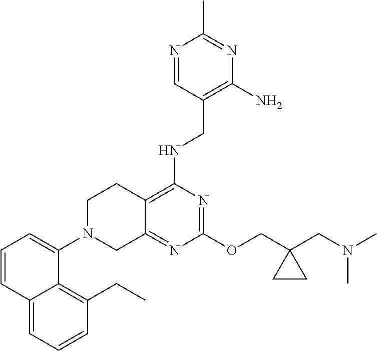 [SMILES: CCc1cccc2cccc(N3CCc4c(nc(OCC5(CN(C)C)CC5)nc4NCc4cnc(C)nc4N)C3)c12]

### N-((4-amino-2-methylpyrimidin-5-yl)methyl)-2-((1-((dimethylamino)methyl)cyclopropyl)methoxy)-7-(8-ethylnaphthalen-1-yl)-5,6,7,8-tetrahydropyrido[3,4-d]pyrimidin-4-amine

### Example 678

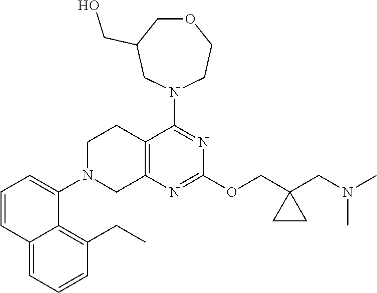 [SMILES: CCc1cccc2cccc(N3CCc4c(nc(OCC5(CN(C)C)CC5)nc4N4CCOCC(CO)C4)C3)c12]

### (4-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)-1,4-oxazepan-6-yl)methanol

### Example 679

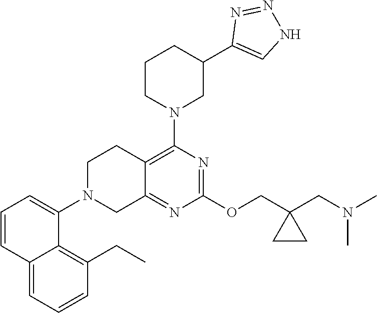 [SMILES: CCc1cccc2cccc(N3CCc4c(nc(OCC5(CN(C)C)CC5)nc4N4CCCC(c5c[nH]nn5)C4)C3)c12]

### 1-(1-(((4-(3-(1H-1,2,3-triazol-4-yl)piperidin-1-yl)-7-(8-ethylnaphthalen-1-yl)-5,6,7,8-tetrahydropyrido[3,4-d]pyrimidin-2-yl)oxy)methyl)cyclopropyl)-N,N-dimethylmethanamine

### Example 680

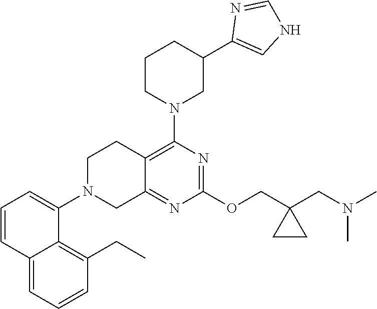 [SMILES: CCc1cccc2cccc(N3CCc4c(nc(OCC5(CN(C)C)CC5)nc4N4CCCC(c5c[nH]cn5)C4)C3)c12]

### 1-(1-(((4-(3-(1H-imidazol-4-yl)piperidin-1-yl)-7-(8-ethylnaphthalen-1-yl)-5,6,7,8-tetrahydropyrido[3,4-d]pyrimidin-2-yl)oxy)methyl)cyclopropyl)-N,N-dimethylmethanamine

### Example 681

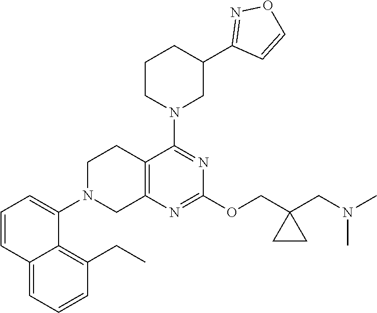 [SMILES: CCc1cccc2cccc(N3CCc4c(nc(OCC5(CN(C)C)CC5)nc4N4CCCC(c5ccon5)C4)C3)c12]

### 1-(1-(((7-(8-ethylnaphthalen-1-yl)-4-(3-(isoxazol-3-yl)piperidin-1-yl)-5,6,7,8-tetrahydropyrido[3,4-d]pyrimidin-2-yl)oxy)methyl)cyclopropyl)-N,N-dimethylmethanamine

### Example 682

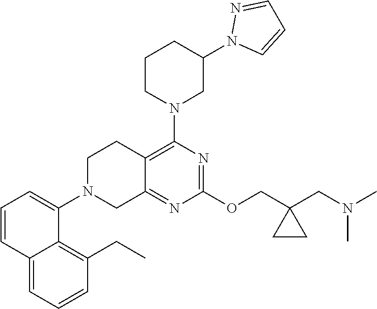 [SMILES: CCc1cccc2cccc(N3CCc4c(nc(OCC5(CN(C)C)CC5)nc4N4CCCC(n5cccn5)C4)C3)c12]

### 1-(1-(((4-(3-(1H-pyrazol-1-yl)piperidin-1-yl)-7-(8-ethylnaphthalen-1-yl)-5,6,7,8-tetrahydropyrido[3,4-d]pyrimidin-2-yl)oxy)methyl)cyclopropyl)-N,N-dimethylmethanamine

### Example 683

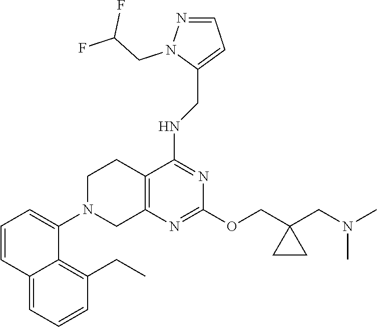 [SMILES: CCc1cccc2cccc(N3CCc4c(nc(OCC5(CN(C)C)CC5)nc4NCc4ccnn4CC(F)F)C3)c12]

### N-((1-(2,2-difluoroethyl)-1H-pyrazol-5-yl)methyl)-2-((1-((dimethylamino)methyl)cyclopropyl)methoxy)-7-(8-ethylnaphthalen-1-yl)-5,6,7,8-tetrahydropyrido[3,4-d]pyrimidin-4-amine

### Example 684

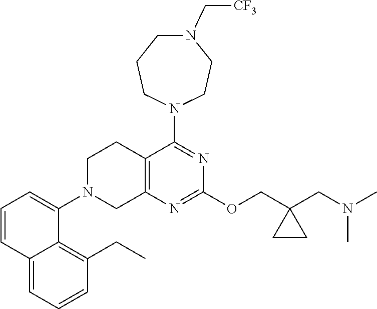 [SMILES: CCc1cccc2cccc(N3CCc4c(nc(OCC5(CN(C)C)CC5)nc4N4CCCN(CC(F)(F)F)CC4)C3)c12]

### 1-(1-(((7-(8-ethylnaphthalen-1-yl)-4-(4-(2,2,2-trifluoroethyl)-1,4-diazepan-1-yl)-5,6,7,8-tetrahydropyrido[3,4-d]pyrimidin-2-yl)oxy)methyl)cyclopropyl)-N,N-dimethylmethanamine

### Example 685

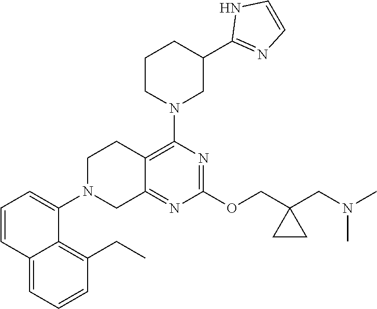 [SMILES: CCc1cccc2cccc(N3CCc4c(nc(OCC5(CN(C)C)CC5)nc4N4CCCC(c5ncc[nH]5)C4)C3)c12]

### 1-(1-(((4-(3-(1H-imidazol-2-yl)piperidin-1-yl)-7-(8-ethylnaphthalen-1-yl)-5,6,7,8-tetrahydropyrido[3,4-d]pyrimidin-2-yl)oxy)methyl)cyclopropyl)-N,N-dimethylmethanamine

### Example 686

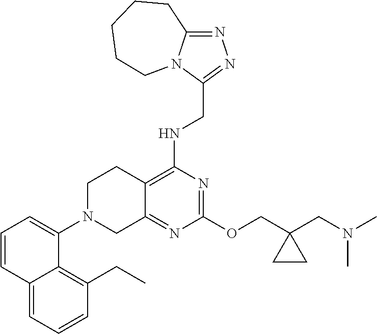 [SMILES: CCc1cccc2cccc(N3CCc4c(nc(OCC5(CN(C)C)CC5)nc4NCc4nnc5n4CCCCC5)C3)c12]

### 2-((1-((dimethylamino)methyl)cyclopropyl)methoxy)-7-(8-ethylnaphthalen-1-yl)-N-((6,7,8,9-tetrahydro-5H-[1,2,4]triazolo[4,3-a]azepin-3-yl)methyl)-5,6,7,8-tetrahydropyrido[3,4-d]pyrimidin-4-amine

### Example 687

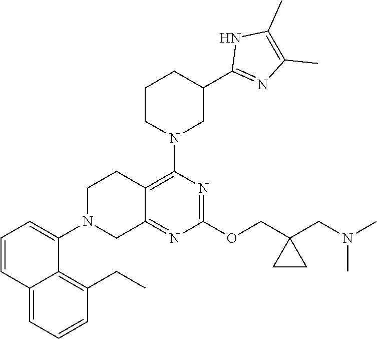 [SMILES: CCc1cccc2cccc(N3CCc4c(nc(OCC5(CN(C)C)CC5)nc4N4CCCC(c5nc(C)c(C)[nH]5)C4)C3)c12]

### 1-(1-(((4-(3-(4,5-dimethyl-1H-imidazol-2-yl)piperidin-1-yl)-7-(8-ethylnaphthalen-1-yl)-5,6,7,8-tetrahydropyrido[3,4-d]pyrimidin-2-yl)oxy)methyl)cyclopropyl)-N,N-dimethylmethanamine

### Example 688

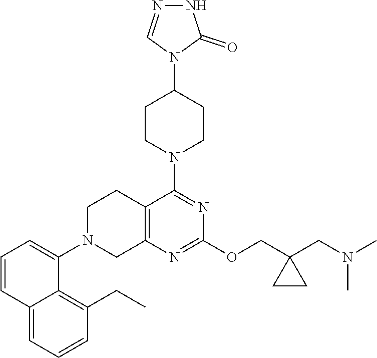 [SMILES: CCc1cccc2cccc(N3CCc4c(nc(OCC5(CN(C)C)CC5)nc4N4CCC(n5cn[nH]c5=O)CC4)C3)c12]

### 4-(1-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)piperidin-4-yl)-2,4-dihydro-3H-1,2,4-triazol-3-one

### Example 689

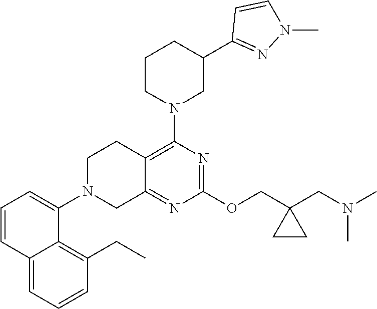 [SMILES: CCc1cccc2cccc(N3CCc4c(nc(OCC5(CN(C)C)CC5)nc4N4CCCC(c5ccn(C)n5)C4)C3)c12]

### 1-(1-(((7-(8-ethylnaphthalen-1-yl)-4-(3-(1-methyl-1H-pyrazol-3-yl)piperidin-1-yl)-5,6,7,8-tetrahydropyrido[3,4-d]pyrimidin-2-yl)oxy)methyl)cyclopropyl)-N,N-dimethylmethanamine

### Example 690

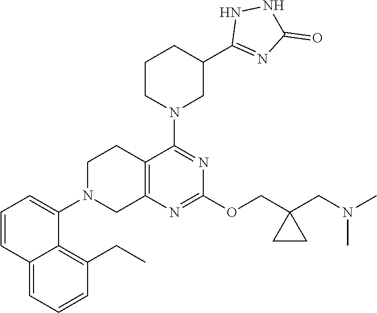 [SMILES: CCc1cccc2cccc(N3CCc4c(nc(OCC5(CN(C)C)CC5)nc4N4CCCC(c5nc(=O)[nH][nH]5)C4)C3)c12]

### 5-(1-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)piperidin-3-yl)-1,2-dihydro-3H-1,2,4-triazol-3-one

### Example 691

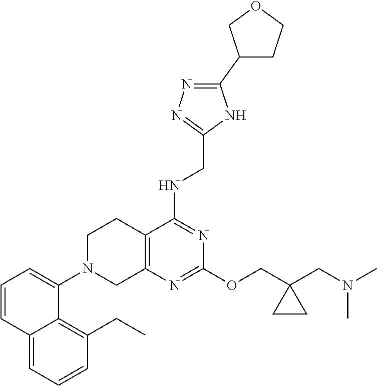 [SMILES: CCc1cccc2cccc(N3CCc4c(nc(OCC5(CN(C)C)CC5)nc4NCc4nnc(C5CCOC5)[nH]4)C3)c12]

### 2-((1-((dimethylamino)methyl)cyclopropyl)methoxy)-7-(8-ethylnaphthalen-1-yl)-N-((5-(tetrahydrofuran-3-yl)-4H-1,2,4-triazol-3-yl)methyl)-5,6,7,8-tetrahydropyrido[3,4-d]pyrimidin-4-amine

### Example 692

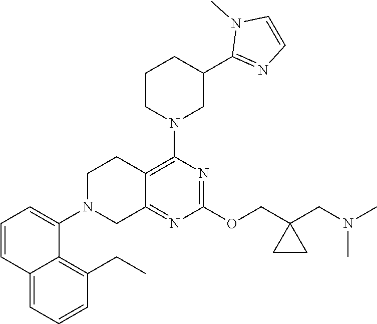 [SMILES: CCc1cccc2cccc(N3CCc4c(nc(OCC5(CN(C)C)CC5)nc4N4CCCC(c5nccn5C)C4)C3)c12]

### 1-(1-(((7-(8-ethylnaphthalen-1-yl)-4-(3-(1-methyl-1H-imidazol-2-yl)piperidin-1-yl)-5,6,7,8-tetrahydropyrido[3,4-d]pyrimidin-2-yl)oxy)methyl)cyclopropyl)-N,N-dimethylmethanamine

### Example 693

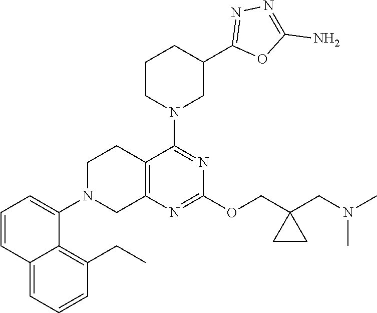 [SMILES: CCc1cccc2cccc(N3CCc4c(nc(OCC5(CN(C)C)CC5)nc4N4CCCC(c5nnc(N)o5)C4)C3)c12]

### 5-(1-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)piperidin-3-yl)-1,3,4-oxadiazol-2-amine

### Example 694

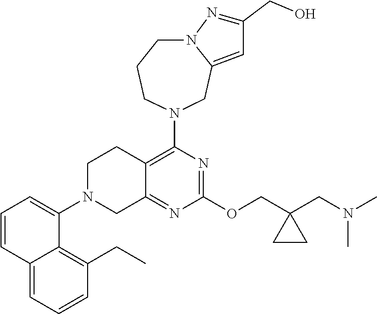 [SMILES: CCc1cccc2cccc(N3CCc4c(nc(OCC5(CN(C)C)CC5)nc4N4CCCn5nc(CO)cc5C4)C3)c12]

### (5-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)-5,6,7,8-tetrahydro-4H-pyrazolo[1,5-a][1,4]diazepin-2-yl)methanol

### Example 695

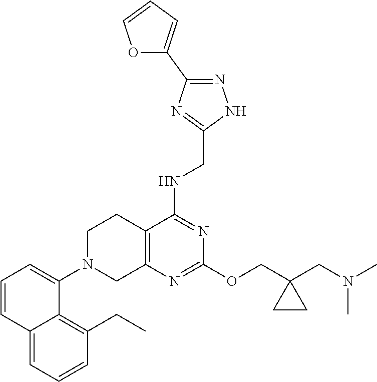 [SMILES: CCc1cccc2cccc(N3CCc4c(nc(OCC5(CN(C)C)CC5)nc4NCc4nc(-c5ccco5)n[nH]4)C3)c12]

### 2-((1-((dimethylamino)methyl)cyclopropyl)methoxy)-7-(8-ethylnaphthalen-1-yl)-N-((3-(furan-2-yl)-1H-1,2,4-triazol-5-yl)methyl)-5,6,7,8-tetrahydropyrido[3,4-d]pyrimidin-4-amine

### Example 696

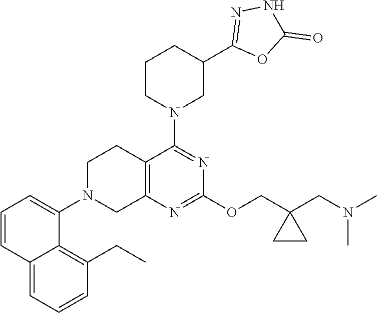 [SMILES: CCc1cccc2cccc(N3CCc4c(nc(OCC5(CN(C)C)CC5)nc4N4CCCC(c5n[nH]c(=O)o5)C4)C3)c12]

### 5-(1-(2-((1-((dimethylamino)methyl)cyclopropyl)methoxy)-7-(8-ethylnaphthalen-1-yl)-5,6,7,8-tetrahydropyrido[3,4-d]pyrimidin-4-yl)piperidin-3-yl)-1,3,4-oxadiazol-2(3H)-one

### Example 697

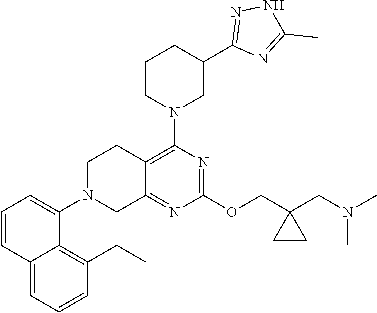 [SMILES: CCc1cccc2cccc(N3CCc4c(nc(OCC5(CN(C)C)CC5)nc4N4CCCC(c5n[nH]c(C)n5)C4)C3)c12]

### 1-(1-(((7-(8-ethylnaphthalen-1-yl)-4-(3-(5-methyl-1H-1,2,4-triazol-3-yl)piperidin-1-yl)-5,6,7,8-tetrahydropyrido[3,4-d]pyrimidin-2-yl)oxy)methyl)cyclopropyl)-N,N-dimethylmethanamine

### Example 698

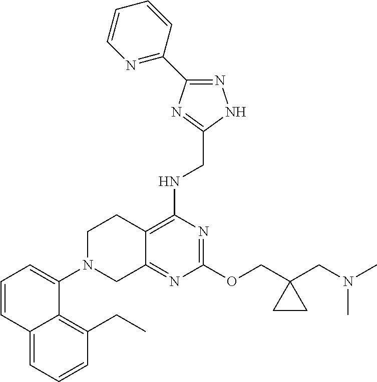 [SMILES: CCc1cccc2cccc(N3CCc4c(nc(OCC5(CN(C)C)CC5)nc4NCc4nc(-c5ccccn5)n[nH]4)C3)c12]

### 2-((1-((dimethylamino)methyl)cyclopropyl)methoxy)-7-(8-ethylnaphthalen-1-yl)-N-((3-(pyridin-2-yl)-1H-1,2,4-triazol-5-yl)methyl)-5,6,7,8-tetrahydropyrido[3,4-d]pyrimidin-4-amine

### Example 699

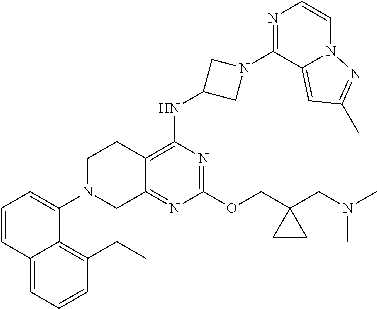 [SMILES: CCc1cccc2cccc(N3CCc4c(nc(OCC5(CN(C)C)CC5)nc4NC4CN(c5nccn6nc(C)cc56)C4)C3)c12]

### 2-((1-((dimethylamino)methyl)cyclopropyl)methoxy)-7-(8-ethylnaphthalen-1-yl)-N-(1-(2-methylpyrazolo[1,5-a]pyrazin-4-yl)azetidin-3-yl)-5,6,7,8-tetrahydropyrido[3,4-d]pyrimidin-4-amine

### Example 700

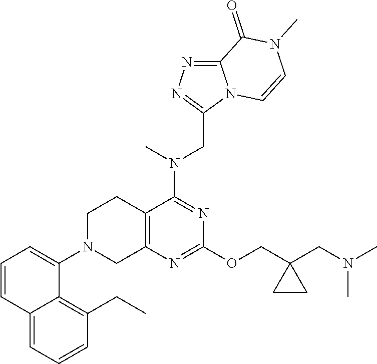 [SMILES: CCc1cccc2cccc(N3CCc4c(nc(OCC5(CN(C)C)CC5)nc4N(C)Cc4nnc5c(=O)n(C)ccn45)C3)c12]

### 3-(((2-((1-((dimethylamino)methyl)cyclopropyl)methoxy)-7-(8-ethylnaphthalen-1-yl)-5,6,7,8-tetrahydropyrido[3,4-d]pyrimidin-4-yl)(methyl)amino)methyl)-7-methyl-[1,2,4]triazolo[4,3-a]pyrazin-8(7H)-one

### Example 701

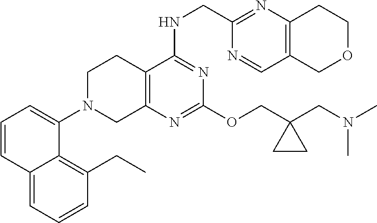 [SMILES: CCc1cccc2cccc(N3CCc4c(nc(OCC5(CN(C)C)CC5)nc4NCc4ncc5c(n4)CCOC5)C3)c12]

### N-((7,8-dihydro-5H-pyrano[4,3-d]pyrimidin-2-yl)methyl)-2-((1-((dimethylamino)methyl)cyclopropyl)methoxy)-7-(8-ethylnaphthalen-1-yl)-5,6,7,8-tetrahydropyrido[3,4-d]pyrimidin-4-amine

### Example 702

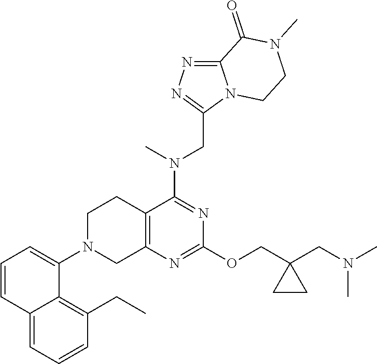 [SMILES: CCc1cccc2cccc(N3CCc4c(nc(OCC5(CN(C)C)CC5)nc4N(C)Cc4nnc5n4CCN(C)C5=O)C3)c12]

### 3-(((2-((1-((dimethylamino)methyl)cyclopropyl)methoxy)-7-(8-ethylnaphthalen-1-yl)-5,6,7,8-tetrahydropyrido[3,4-d]pyrimidin-4-yl)(methyl)amino)methyl)-7-methyl-6,7-dihydro-[1,2,4]triazolo[4,3-a]pyrazin-8(5H)-one

### Example 703

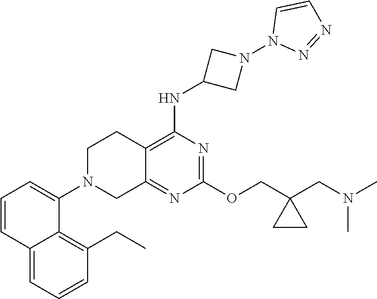 [SMILES: CCc1cccc2cccc(N3CCc4c(nc(OCC5(CN(C)C)CC5)nc4NC4CN(n5ccnn5)C4)C3)c12]

### N-(3-(1H-1,2,3-triazol-1-yl)cyclobutyl)-2-((1-((dimethylamino)methyl)cyclopropyl)methoxy)-7-(8-ethylnaphthalen-1-yl)-5,6,7,8-tetrahydropyrido[3,4-d]pyrimidin-4-amine

### Example 704

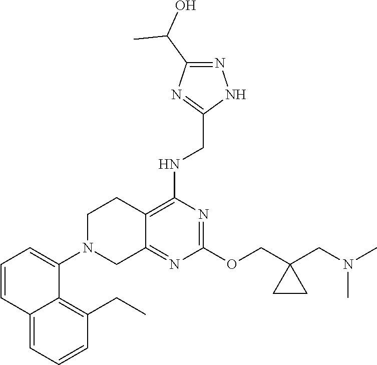 [SMILES: CCc1cccc2cccc(N3CCc4c(nc(OCC5(CN(C)C)CC5)nc4NCc4nc(C(C)O)n[nH]4)C3)c12]

### 1-(5-(((2-((1-((dimethylamino)methyl)cyclopropyl)methoxy)-7-(8-ethylnaphthalen-1-yl)-5,6,7,8-tetrahydropyrido[3,4-d]pyrimidin-4-yl)amino)methyl)-1H-1,2,4-triazol-3-yl)ethan-1-ol

### Example 705

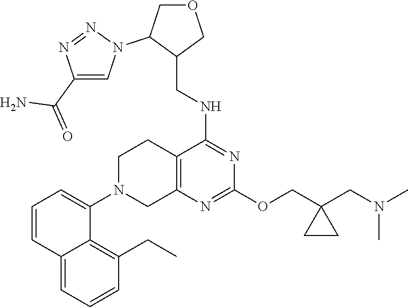 [SMILES: CCc1cccc2cccc(N3CCc4c(nc(OCC5(CN(C)C)CC5)nc4NCC4COCC4n4cc(C(N)=O)nn4)C3)c12]

### 1-(4-(((2-((1-((dimethylamino)methyl)cyclopropyl)methoxy)-7-(8-ethylnaphthalen-1-yl)-5,6,7,8-tetrahydropyrido[3,4-d]pyrimidin-4-yl)amino)methyl)tetrahydrofuran-3-yl)-1H-1,2,3-triazole-4-carboxamide

### Example 706

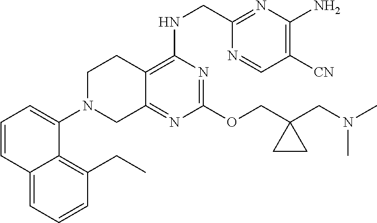 [SMILES: CCc1cccc2cccc(N3CCc4c(nc(OCC5(CN(C)C)CC5)nc4NCc4ncc(C#N)c(N)n4)C3)c12]

### 4-amino-2-(((2-((1-((dimethylamino)methyl)cyclopropyl)methoxy)-7-(8-ethylnaphthalen-1-yl)-5,6,7,8-tetrahydropyrido[3,4-d]pyrimidin-4-yl)amino)methyl)pyrimidine-5-carbonitrile

### Example 707

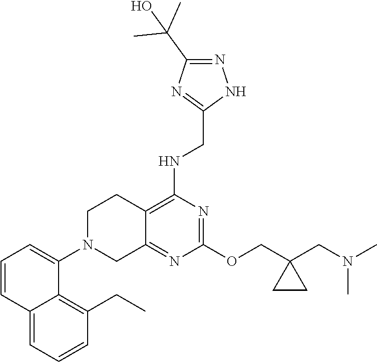 [SMILES: CCc1cccc2cccc(N3CCc4c(nc(OCC5(CN(C)C)CC5)nc4NCc4nc(C(C)(C)O)n[nH]4)C3)c12]

### 2-(5-(((2-((1-((dimethylamino)methyl)cyclopropyl)methoxy)-7-(8-ethylnaphthalen-1-yl)-5,6,7,8-tetrahydropyrido[3,4-d]pyrimidin-4-yl)amino)methyl)-1H-1,2,4-triazol-3-yl)propan-2-ol

### Example 708

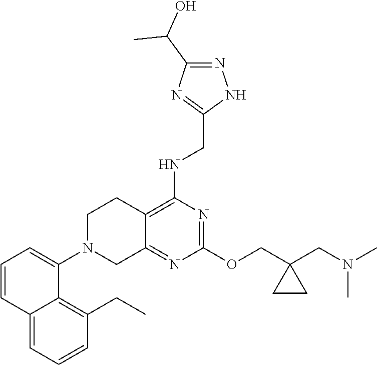 [SMILES: CCc1cccc2cccc(N3CCc4c(nc(OCC5(CN(C)C)CC5)nc4NCc4nc(C(C)O)n[nH]4)C3)c12]

### 1-(5-(((2-((1-((dimethylamino)methyl)cyclopropyl)methoxy)-7-(8-ethylnaphthalen-1-yl)-5,6,7,8-tetrahydropyrido[3,4-d]pyrimidin-4-yl)amino)methyl)-1H-1,2,4-triazol-3-yl)ethan-1-ol

### Example 709

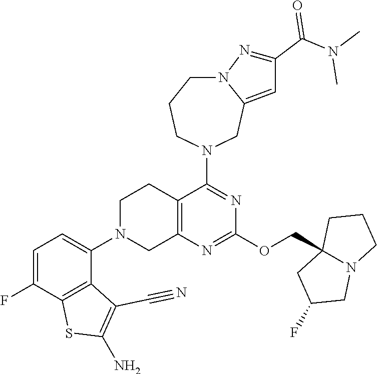 [SMILES: CN(C)C(=O)c1cc2n(n1)CCCN(c1nc(OC[C@@]34CCCN3C[C@H](F)C4)nc3c1CCN(c1ccc(F)c4sc(N)c(C#N)c14)C3)C2]

### 5-(7-(2-amino-3-cyano-7-fluorobenzo[b]thiophen-4-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide

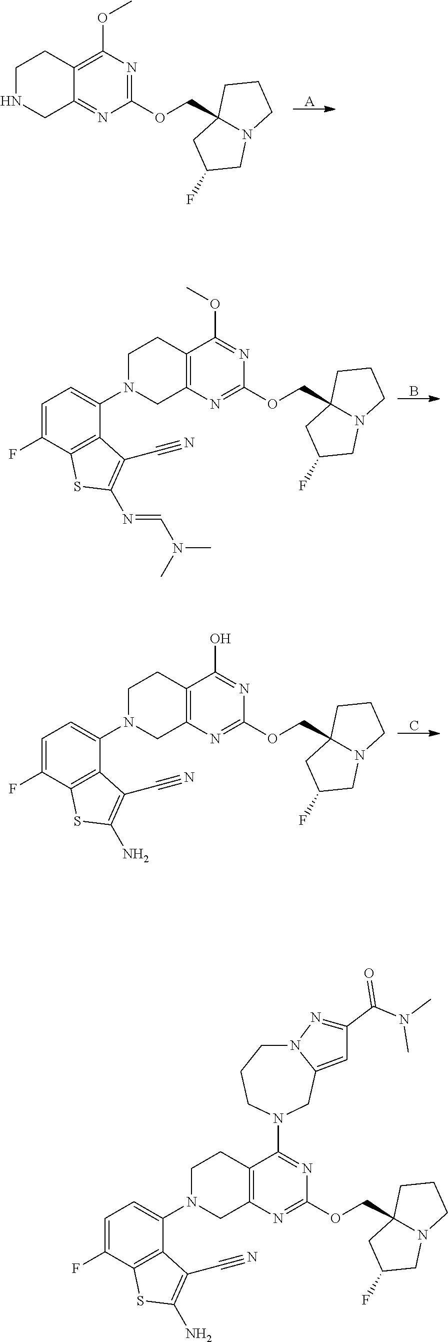

Step A. (E)-N′-(3-cyano-7-fluoro-4-(2-(((2R,7aS)-2-fluorotetrahydro-1H-pyrrolizin-7a(5H)-yl)methoxy)-4-methoxy-5,8-dihydropyrido[3,4-d]pyrimidin-7(6H)-yl)benzo[b]thiophen-2-yl)-N,N-dimethylformimidamide: To a mixture of 2-(((2R,7aS)-2-fluorotetrahydro-1H-pyrrolizin-7a(5H)-yl)methoxy)-4-methoxy-5,6,7,8-tetrahydropyrido[3,4-d]pyrimidine (1.00 g, 1.0 equiv) and (E)-N′-(4-bromo-3-cyano-7-fluorobenzo[b]thiophen-2-yl)-N,N-dimethylformimidamide (1.21 g, 1.2 equiv) in dioxane (10 mL) were added Cs₂CO₃ (3.03 g, 3.0 equiv) and Pd-PEPPSI-IPentCl (302 mg, 0.1 equiv). The reaction was degassed and purged with nitrogen 3 times. The reaction was stirred at 90° C. for 12 hours. The mixture was diluted with H₂O (10 mL) and extracted with EtOAc (3×10 mL). The combined organic layers were washed with brine (10 mL), dried over anhydrous sodium sulfate, concentrated and purified by reversed phase flash chromatography [C18, 0.1% formic acid condition] to afford the title compound (850 mg, 48% yield) as yellow solid; 1H NMR (400 MHz, CHLOROFORM-d) δ=7.93-7.78 (m, 1H), 6.97-6.84 (m, 2H), 5.39-5.17 (m, 1H), 4.25-3.99 (m, 4H), 3.96 (s, 3H), 3.58-3.40 (m, 1H), 3.39-3.21 (m, 3H), 3.17 (d, J=7.4 Hz, 6H), 3.15-2.88 (m, 3H), 2.62-2.44 (m, 1H), 2.30-2.13 (m, 3H), 1.99-1.82 (m, 3H);

Step B. 2-amino-7-fluoro-4-(2-(((2R,7aS)-2-fluorotetrahydro-1H-pyrrolizin-7a(5H)-yl)methoxy)-4-hydroxy-5,8-dihydropyrido[3,4-d]pyrimidin-7(6H)-yl)benzo[b]thiophene-3-carbonitrile: To a solution of (E)-N′-(3-cyano-7-fluoro-4-(2-(((2R,7aS)-2-fluorotetrahydro-1H-pyrrolizin-7a(5H)-yl)methoxy)-4-methoxy-5,8-dihydropyrido[3,4-d]pyrimidin-7(6H)-yl)benzo[b]thiophen-2-yl)-N,N-dimethylformimidamide (200 mg, 1.0 equiv) in DMF (2 mL) was added NaSEt (88.9 mg, 3.0 equiv). The mixture was degassed and purged with N₂ for 3 times. The reaction was stirred at 60° C. for 12 hours. The mixture was diluted with H₂O (2 mL) and extracted with EtOAc (3×2 mL). The combined organic layers were washed with brine (2 mL), dried over anhydrous sodium sulfate, filtered and concentrated to afford the title compound (140 mg, 59% yield) as yellow solid; LCMS (ESI, M+1): m/z=499.2.

Step C. 5-(7-(2-amino-3-cyano-7-fluorobenzo[b]thiophen-4-yl)-2-(((2R,7aS)-2-fluorotetrahydro-1H-pyrrolizin-7a(5H)-yl)methoxy)-5,6,7,8-tetrahydropyrido[3,4-d] pyrimidin-4-yl)-N,N-dimethyl-5,6,7,8-tetrahydro-4H-pyrazolo[1,5-a][1,4]diazepine-2-carboxamide:

To a solution of 2-amino-7-fluoro-4-(2-(((2R,7aS)-2-fluorotetrahydro-1H-pyrrolizin-7a(5H)-yl)methoxy)-4-hydroxy-5,8-dihydropyrido[3,4-d]pyrimidin-7(6H)-yl)benzo[b]thiophene-3-carbonitrile (40.0 mg, 1.0 equiv), N,N-dimethyl-5,6,7,8-tetrahydro-4H-pyrazolo[1,5-a][1,4]diazepine-2-carboxamide (29.5 mg, 1.5 equiv, HCl) and DIEA (33.1 mg, 3.0 equiv) in DMF (1 mL) was added PyBOP (62.6 mg, 1.5 equiv). The reaction was stirred at 25° C. for 12 hours. The mixture was diluted with H₂O (10 mL) and extracted with EtOAc (3×2 mL). The combined organic layers were washed with brine (2 mL), dried over anhydrous sodium sulfate, filtered and purified by prep-HPLC (Waters Xbridge C18 150×25 mm×5 μm; A: water (10 mM NH₃•H₂O); B: ACN, B %: 30%-60% over 10 min) to afford the title compound (3.32 mg, 5.8% yield) as yellow solid. ¹H NMR (400 MHz, CHLOROFORM-d) δ=7.00 (dd, J=4.4, 8.8 Hz, 1H), 6.9-6.82 (m, 1H), 6.66 (s, 1H), 5.82 (br s, 2H), 5.38-5.16 (m, 1H), 4.87-4.54 (m, 2H), 4.48 (br s, 2H), 4.17-4.04 (m, 3H), 4.00-3.86 (m, 3H), 3.35 (s, 5H), 3.27-3.13 (m, 4H), 3.11 (s, 3H), 3.03-2.85 (m, 2H), 2.15 (br d, J=13.6 Hz, 5H), 1.97-1.83 (m, 3H); LCMS (ESI, M+1): m/z=689.5.

### Example 710

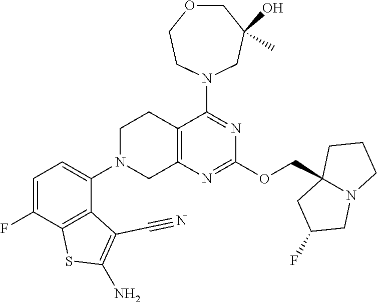 [SMILES: C[C@@]1(O)COCCN(c2nc(OC[C@@]34CCCN3C[C@H](F)C4)nc3c2CCN(c2ccc(F)c4sc(N)c(C#N)c24)C3)C1]

### 2-amino-7-fluoro-4-(2-(((2R,7aS)-2-fluorotetrahydro-1H-pyrrolizin-7a(5H)-yl)methoxy)-4-((S)-6-hydroxy-6-methyl-1,4-oxazepan-4-yl)-5,8-dihydropyrido[3,4-d]pyrimidin-7(6H)-yl)benzo[b]thiophene-3-carbonitrile

Synthesized according to Example 709. The title compound was obtained as as yellow solid. ¹H NMR (400 MHz, CHLOROFORM-d) δ=7.10-6.98 (m, 1H), 6.93-6.84 (m, 1H), 5.46-5.38 (m, 2H), 5.38-5.20 (m, 1H), 4.24-4.00 (m, 6H), 3.89 (br d, J=12.8 Hz, 1H), 3.69-3.57 (m, 2H), 3.43-3.27 (m, 6H), 3.27-3.13 (m, 2H), 3.13-2.73 (m, 3H), 2.32-2.14 (m, 2H), 2.09 (br d, J=7.3 Hz, 1H), 2.00-1.88 (m, 3H), 1.30 (s, 3H); LCMS (ESI, M+1): m/z=612.4.

### Example 711

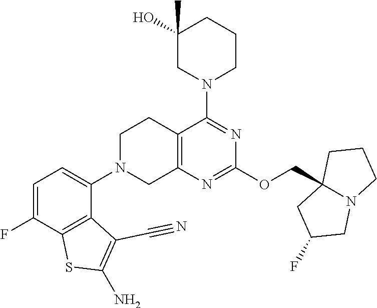 [SMILES: C[C@@]1(O)CCCN(c2nc(OC[C@@]34CCCN3C[C@H](F)C4)nc3c2CCN(c2ccc(F)c4sc(N)c(C#N)c24)C3)C1]

### 2-amino-7-fluoro-4-(2-(((2R,7aS)-2-fluorotetrahydro-1H-pyrrolizin-7a(5H)-yl)methoxy)-4-((R)-3-hydroxy-3-methylpiperidin-1-yl)-5,8-dihydropyrido[3,4-d]pyrimidin-7(6H)-yl)benzo[b]thiophene-3-carbonitrile

Synthesized according to Example 709, The title compound was obtained as off-white solid. ¹H NMR (400 MHz, METHANOL-d₄) δ=7.10 (dd, J=4.4, 8.8 Hz, 1H), 6.87 (t, J=8.8 Hz, 1H), 5.37-5.18 (m, 1H), 4.23-4.00 (m, 3H), 3.96-3.45 (m, 4H), 3.44-3.33 (m, 2H), 3.26-3.12 (m, 4H), 2.99 (dt, J=6.0, 9.2 Hz, 2H), 2.71-2.44 (m, 1H), 2.22 (s, 1H), 2.21-2.14 (m, 1H), 2.13-2.07 (m, 1H), 2.02-1.80 (m, 4H), 1.78-1.58 (m, 3H), 1.22 (br s, 3H); LCMS (ESI, M+1): m/z=596.4

### Example 712

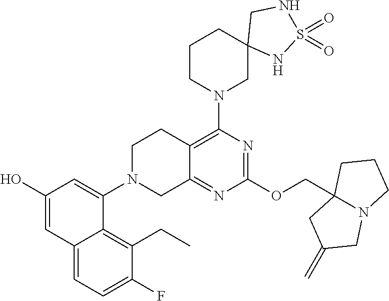 [SMILES: C=C1CN2CCCC2(COc2nc3c(c(N4CCCC5(CNS(=O)(=O)N5)C4)n2)CCN(c2cc(O)cc4ccc(F)c(CC)c24)C3)C1]

7-(7-(8-ethyl-7-fluoro-3-hydroxynaphthalen-1-yl)-2-((2-methylenetetrahydro-1H-pyrrolizin-7a(5H)-yl)methoxy)-5,6,7,8-tetrahydropyrido[3,4-d]pyrimidin-4-yl)-2-thia-1,3,7-triazaspiro[4.5]decane 2,2-dioxide

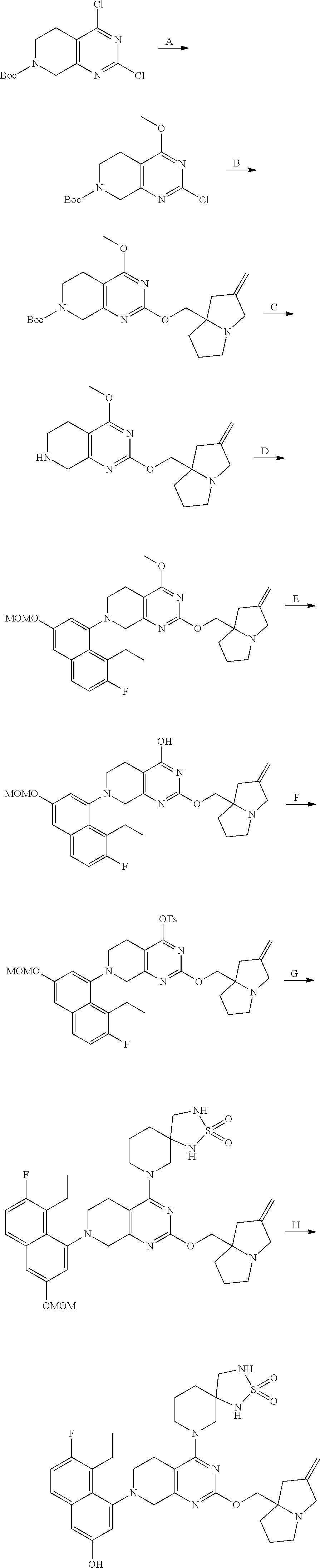

Step A. tert-butyl 2-chloro-4-methoxy-5,8-dihy dropyrido[3,4-d]pyrimidine-7(6H)-carboxylate: To a solution of tert-butyl 2,4-dichloro-5,8-dihydropyrido[3,4-d]pyrimidine-7(6H) -carboxylate (20.0 g, 1.0 equiv) in MeOH (200 mL) was added NaOMe (23.7 g, 30% purity, 2.0 equiv) at 0° C. The reaction was stirred at 0° C. for 3 hours. The mixture was diluted with water (400 mL) and extracted with ethyl acetate (2×500 mL). The combined organic layers were washed with brine (300 mL), dried over anhydrous sodium sulfate, concentrated, and purified with column chromatography [SiO2, petroleum ether/ethyl acetate=50/1 to 10/1] to afford the title compound (14.5 g, 69% yield) as white solid;, LCMS (ESI, M+1): m/z=300.1.

Step B. tert-butyl 4-methoxy-2-((2-methylenetetrahydro -1H-pyrrolizin -7a(5H)-yl)methoxy)-5, 8-dihydropyri do[3,4-d]pyrimidine-7(6H)-carboxylate: To a solution of (2-methylenetetrahydro-1H-pyrrolizin-7a(5H)-yl)methanol (4.40 g, 1.0 equiv) and tert-butyl 2-chloro-4-methoxy-5,8-dihydropyrido[3,4-d]pyrimidine-7(6H)-carboxylate (8.60 g, 1.0 equiv) in toluene (100 mL) were added Pd(OAc)₂ (644 mg, 0.1 equiv), BINAP (3.57 g, 0.2 equiv) and Cs₂CO₃ (28.0 g, 3.0 equiv). The reaction was degassed and purged with nitrogen 3 times. The reaction was stirred at 110° C. for 2 hours. The mixture was diluted with water (200 mL) and extracted with ethyl acetate (2×300 mL). The combined organic layers were washed with brine (100 mL), dried over anhydrous sodium sulfate, concentrated, and purified with column chromatography [SiO₂, petroleum ether/ethyl acetate=1/1 to 10/1] and prep-HPLC [0.1% FA condition] to afford the title compound (6.30 g, 53% yield) as yellow oil; LCMS (ESI, M+1): m/z=417.1.

Step C. 4-methoxy-2-((2-methylenetetrahydro-1H-pyrrolizin-7a(5H)-yl)methoxy)-5,6,7,8-tetrahydropyrido[3,4-d]pyrimidine: To a solution of tert-butyl 4-methoxy-2-((2-methylenetetrahydro-1H-pyrrolizin-7a(5H)-yl)methoxy)-5,8-dihydropyrido[3,4-d]pyrimidine-7(6H)-carboxylate (6.00 g, 1.0 equiv) in MeOH (30 mL) was added HCl•MeOH (4M, 30 mL). The reaction was stirred at 25° C. for 5 hours. The mixture was concentrated. The residue was diluted with DCM/MeOH=10/1 (60 mL) and washed with sodium hydroxide solution (1 M, 4×80 mL). The organic layer was washed with brine (40 mL), dried over anhydrous sodium sulfate and concentrated to afford the title compound (4.30 g, 94% yield) as yellow gum; LCMS (ESI, M+1): m/z=317.1.

Step D. 7-(8-ethyl-7-fluoro-3-(methoxymethoxy)naphthalen-1-yl)-4-methoxy-2-((2-methylenehexahydro-1H-pyrrolizin-7a-yl)methoxy)-5,6,7,8-tetrahydropyrido[3,4-d]pyrimidine: To a solution of 4-methoxy-2-((2-methylenetetrahydro-1H-pyrrolizin-7a(5H)-yl)methoxy)-5,6,7,8-tetrahydropyrido[3,4-d]pyrimidine (3.30 g, 1.0 equiv) and 8-ethyl-7-fluoro-3-(methoxymethoxy)naphthalen-1-yl trifluoromethanesulfonate (4.79 g, 1.2 equiv) in toluene (5⁵ mL) were added Pd₂(dba)₃ (955 mg, 0.1 equiv), Xantphos (1.21 g, 0.2 equiv) and Cs₂CO₃ (10.2 g, 3.0 equiv). The reaction was degassed and purged with nitrogen 3 times. The reaction was stirred at 110° C. for 12 hours. The mixture was diluted with water (80 mL) and extracted with ethyl acetate (2×100 mL). The combined organic layers were washed with brine (60 mL), dried over anhydrous sodium sulfate, concentrated, and purified with column chromatography [SiO₂, petroleum ether:ethyl acetate=3:1 to 0:1; dichloromethane: methanol=10:1 to 0:1] and prep-HPLC [column: Phenomenex luna C18 (250×70 mm, 10 μm); mobile phase: water(FA)-ACN; gradient:30%-60% B over 20 min] to afford the title compound (1.15 g, 20% yield) as red solid; LCMS (ESI, M+1): m/z=549.3.

Step E. 7-(8-ethyl-7-fluoro-3-(methoxymethoxy)naphthalen-1-yl)-2-((2-methylenetetrahydro -1H-pyrrolizin-7a(5H)-yl)methoxy)-5,6,7,8-tetrahydropyrido[3,4-d] pyrimidin-4-ol: To a solution of 7-(8-ethyl-7-fluoro-3-(methoxymethoxy)naphthalen-1-yl)-4-methoxy-2-((2-methylenetetrahydro-1H-pyrrolizin-7a(5H)-yl)methoxy)-5,6,7,8-tetrahydropyrido[3,4-d]pyrimidine (1.14 g, 1.0 equiv) in DMAc (20 mL) was added NaSEt (874 mg, 5.0 equiv). The reaction was stirred at 60° C. for 0.5 hours. The mixture was diluted with water (100 mL) and extracted with ethyl acetate (3×80 mL). The combined organic layers were washed with brine (100 mL), dried over anhydrous sodium sulfate, concentrated and purified with prep-HPLC [column: Waters Xbridge 150×25 mm×5 μm; mobile phase: water ammonia hydroxide v/v-ACN; gradient:40%-70% B over min] to afford the title compound (784 mg, 67% yield) as yellow solid; LCMS (ESI, M+1): m/z=535.2.

Step F. 7-(8-ethyl-7-fluoro-3-(methoxymethoxy)naphthalen-1-yl)-2-((2-methylenehexahydro-1H-pyrrolizin-7a-yl)methoxy)-5,6,7,8-tetrahydropyrido[3,4-d] pyrimidin-4-yl 4-methylbenzenesulfonate: To a solution of 7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d]pyrimidin-4-ol (340 mg, 1.0 equiv) and TEA (193 mg, 3.0 equiv) in DCM (4.5 mL) were added 4-methylbenzenesulfonyl chloride (182 mg, 1.5 equiv) and DMAP (15.5 mg, 0.2 equiv) at 0° C. The reaction was stirred at 25° C. for 1 hour. The mixture was diluted with water (10 mL) and extracted with DCM (2×15 mL). The combined organic layers were washed with brine (10 mL), dried over anhydrous sodium sulfate, concentrated and purified with column chromatography [SiO₂, petroleum ether/ethyl acetate=0/1] to afford the title compound (374 mg, 81% yield) as yellow oil; LCMS (ESI, M+1): m/z=689.3.

Step G. 7-(7-(8-ethyl-7-fluoro-3-(methoxymethoxy)naphthalen-1-yl)-2-((2-methylenehexahydro-1H-pyrrolizin-7a-yl)methoxy)-5,6,7,8-tetrahydropyrido[3,4-d]pyrimidin-4-yl)-2-thia-1,3,7-triazaspiro[4.5]decane 2,2-dioxide: To a solution of 7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d]pyrimidin-4-yl 4-methylbenzenesulfonate (60.0 mg, 1.0 equiv) and DIEA (56.3 mg, 5.0 equiv) in DMF (3 mL) were added 2-thia-1,3,7-triazaspiro[4.5]decane 2,2-dioxide (33.3 mg, 2.0 equiv) and 4 Å molecular sieve (20.0 mg). The reaction was stirred at 80° C. for 12 hours. The mixture was diluted with water (5 mL) and extracted with ethyl acetate (2×5 mL). The combined organic layers were washed with brine (5 mL), dried over anhydrous sodium sulfate, concentrated and purified with prep-TLC [SiO2, DCM: MeOH=10:1] to afford the title compound (40.0 mg, 65% yield) as brown solid; LCMS (ESI, M+1): m/z=708.3.

Step H. 7-(7-(8-ethyl-7-fluoro-3-hydroxynaphthalen-1-yl)-2-((2-methylenetetrahydro-1H-pyrrolizin-7a(5H)-yl)methoxy)-5,6,7,8-tetrahydropyrido[3,4-d] pyrimidin-4-yl)-2-thia-1,3,7-triazaspiro[4.5]decane 2,2-dioxide: To a solution of 7-(7-(8-ethyl-7-fluoro-3-(methoxymethoxy)naphthalen-1-yl)-2-((2-methylenetetrahydro-1H-pyrrolizin-7a(5H)-yl)methoxy)-5,6,7,8-tetrahydropyrido[3,4-d]pyrimidin-4-yl)-2-thia-1,3,7-triazaspiro[4.5]decane 2,2-dioxide (40.0 mg, 1.0 equiv) in MeOH (1 mL) was added HCl-MeOH (4M, 1 mL) at 0° C. The reaction was stirred at 0° C. for 1 hour. The mixture was concentrated. The residue was diluted with MeOH (2 mL), adjusted to pH=7 with solid NaHCO₃ and filtered. The filtrate was concentrated and purified with prep-HPLC [Waters Xbridge 150×25 mm×5 μm; mobile phase: water(NH₄HCO₃)-ACN; gradient: 42%-72% B over 9 min] to afford the title compound (14.6 mg, 38% yield) as light brown solid; ¹H NMR (400 MHz, METHANOL-d₄) δ=7.53-7.46 (m, 1H), 7.18-7.09 (m, 1H), 7.02-6.93 (m, 2H), 4.95 (s, 2H), 4.20-4.04 (m, 3H), 4.03-3.85 (m, 1H), 3.80-3.51 (m, 4H), 3.51-3.33 (m, 5H), 3.27-3.10 (m, 5H), 2.82-2.60 (m, 3H), 2.47-2.38 (m, 1H), 2.15-2.04 (m, 1H), 2.02-1.72 (m, 7H), 1.16-1.06 (m, 7H); LCMS (ESI, M+1): m/z=664.4.

### Example 713

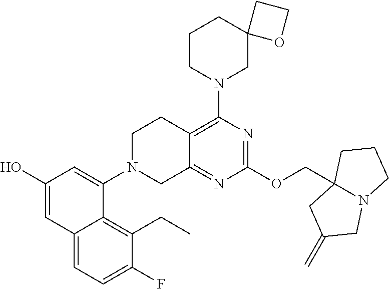 [SMILES: C=C1CN2CCCC2(COc2nc3c(c(N4CCCC5(CCO5)C4)n2)CCN(c2cc(O)cc4ccc(F)c(CC)c24)C3)C1]

### 5-ethyl-6-fluoro-4-(2-((2-methylenetetrahydro-1H-pyrrolizin-7a(5H)-yl)methoxy)-4-(1-oxa-6-azaspiro[3.5]nonan-6-yl)-5,8-dihydropyrido[3,4-d]pyrimidin-7(6H)-yl)naphthalen-2-ol

Synthesized according to Example 712. The target was ontained as brown gum; ¹H NMR (400 MHz, DMSO-d₆) δ=9.69 (s, 1H), 7.59 (dd, J=6.0, 8.8 Hz, 1H), 7.24 (t, J=9.2 Hz, 1H), 6.99 (s, 2H), 4.88 (s, 2H), 4.49-4.26 (m, 2H), 3.96-3.74 (m, 4H), 3.67-3.57 (m, 1H), 3.55-3.45 (m, 2H), 3.44-3.38 (m, 2H), 3.23-3.07 (m, 4H), 3.07-2.93 (m, 2H), 2.69-2.54 (m, 2H), 2.45-2.16 (m, 4H), 2.01-1.88 (m, 2H), 1.87-1.70 (m, 4H), 1.69-1.42 (m, 3H), 1.07 (t, J=6.8 Hz, 3H); LCMS (ESI, M+1): m/z=600.3.

### Example 714

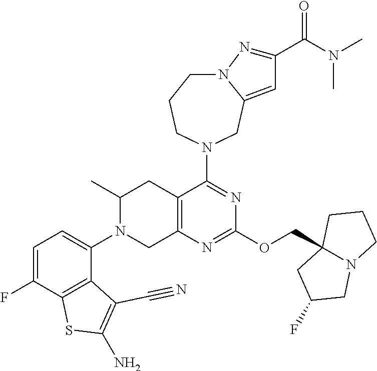 [SMILES: CC1Cc2c(nc(OC[C@@]34CCCN3C[C@H](F)C4)nc2N2CCCn3nc(C(=O)N(C)C)cc3C2)CN1c1ccc(F)c2sc(N)c(C#N)c12]

### 5-(7-(2-amino-3-cyano-7-fluorobenzo[b]thiophen-4-yl)-2-(((2R,7aS)-2-fluorotetrahydro-1H-pyrrolizin-7a(5H)-yl)methoxy)-6-methyl-5,6,7,8-tetrahydropyrido[3,4-d]pyrimidin-4-yl)-N,N-dimethyl-5,6,7,8-tetrahydro-4H-pyrazolo[1,5-a][1,4]diazepine-2-carboxamide

### Example 715

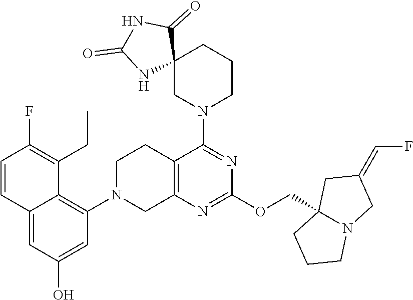 [SMILES: CCc1c(F)ccc2cc(O)cc(N3CCc4c(nc(OC[C@@]56CCCN5C/C(=C\F)C6)nc4N4CCC[C@]5(C4)NC(=O)NC5=O)C3)c12]

### (R)-7-(7-(8-ethyl-7-fluoro-3-hydroxynaphthalen-1-yl)-2-(((S,Z)-2-(fluoromethylene)tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione

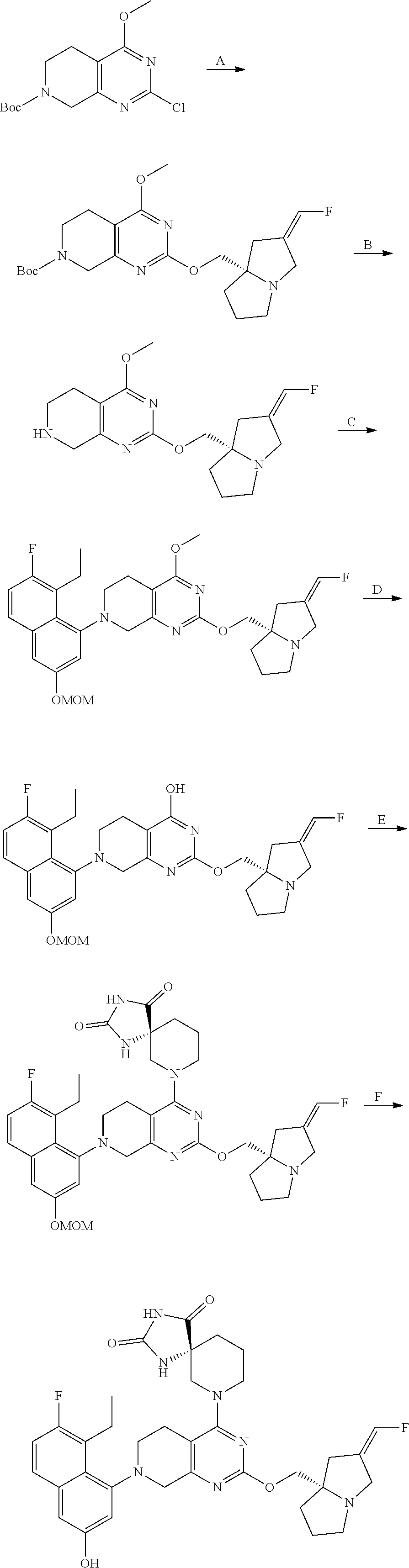

Step A. tert-butyl (S,Z)-2-((2-(fluoromethylene)tetrahydro-1H-pyrrolizin-7a(5H)-yl)methoxy)-4-methoxy-5,8-dihydropyrido[3,4-d] pyrimidine-7(6H)-carboxylate: To a mixture of tert-butyl 2-chloro-4-methoxy-5,8-dihydropyrido[3,4-d]pyrimidine-7(6H)-carboxylate (7.0 g, 1.0 equiv), (S,Z)-(2-(fluoromethylene)tetrahydro-1H-pyrrolizin-7a(5H)-yl)methanol (4.40 g, 1.1 equiv) and Cs₂CO₃ (22.8 g, 3.0 equiv) in toluene (70.0 mL) was added 2,2′-bis(diphenylphosphaneyl)-1,1′-binaphthalene (2.91 g, 0.2 equiv). The mixture was degassed and purged with N₂ for 3 times. diacetoxypalladium (524 mg, 0.10 equiv) was added into the mixture. The reaction was degassed and purged with N₂ for 3 times. The reaction was stirred at 110° C. for 3 hours. The mixture was filtered, concentrated, and purified with prep-HPLC [Phenomenex luna C18 250×70 mm,10 μm; A: water (FA), B: ACN, B %: 22%-50% over 18 min] to afford the title compound (9.20 g, 91% yield) as yellow oil; LCMS (ESI, M+1): m/z=435.2.

Step B. (S,Z)-2-((2-(fluoromethylene)tetrahydro-1H-pyrrolizin-7a(5H)-yl)methoxy)-4-methoxy-5,6,7,8-tetrahydropyrido[3,4-d] pyrimidine: A solution of tert-butyl (S,Z)-2-((2-(fluoromethylene)tetrahydro-1H-pyrrolizin-7a(5H)-yl)methoxy)-4-methoxy-5,8-dihydropyrido[3,4-d]pyrimidine-7(6H)-carboxylate (8.00 g, 1.0 equiv) in HCl-MeOH (4 M, 30 mL, 6.5 equiv) was stirred at 25° C. for 0.5 hours. The mixture was concentrated. The residue was diluted with water (30.0 mL). The pH of the mixture was adjusted to 11 with sodium hydroxide solution (15 ml). The mixture was extracted with ethyl acetate (3×100 mL). The combined organic layers were dried over anhydrous sodium sulfate and concentrated to afford the title compound (6.00 g, 88% yield) as yellow oil; LCMS (ESI, M+1): m/z=335.0.

Step C. (S,Z)-7-(8-ethyl-7-fluoro-3-(methoxymethoxy)naphthalen-1-yl)-2-((2-(fluoromethylene)tetrahydro-1H-pyrrolizin-7a(5H)-yl)methoxy)-4-methoxy-5,6,7,8-tetrahydropyrido[3,4-d]pyrimidine: To a solution of (S,Z)-2-((2-(fluoromethylene)tetrahydro-1H-pyrrolizin-7a(5H)-yl)methoxy)-4-methoxy-5,6,7,8-tetrahydropyrido[3,4-d]pyrimidine (6.00 g, 1.0 equiv) and 8-ethyl-7-fluoro-3-(methoxymethoxy)naphthalen-1-yl trifluoromethanesulfonate (8.92 g, 1.3 equiv) in dioxane (100 mL) was added Cs₂CO₃ (17.5 g, 3.0 equiv). The mixture was degassed and purged with N₂ for 3 times. 1,3-bis[2,6-bis(1-propylbutyl)phenyl]-4,5-dichloro-2H-imidazol-1-ium-2-ide;3-chloropyridine;dichloropalladium (872 mg, 0.05 equiv) was added to the mixture. The reaction was degassed and purged with nitrogen 3 times. The reaction was stirred at 90° C. for 16 hours. The mixture was filtered and purified with reversed phase flash chromatography [C18, 0.1% formic acid condition] to afford the title compound (6.00 g, 57.2% yield) as yellow oil; LCMS (ESI, M+1): m/z=567.2.

Step D. (S,Z)-7-(8-ethyl-7-fluoro-3-(methoxymethoxy)naphthalen-1-yl)-2-((2-(fluoromethylene)tetrahydro-1H-pyrrolizin-7a(5H)-yl)methoxy)-5,6,7,8-tetrahydropyrido[3,4-d] pyrimidin-4-ol: To a solution of (S,Z)-7-(8-ethyl-7-fluoro-3-(methoxymethoxy)naphthalen-1-yl)-2-((2-(fluoromethylene)tetrahydro-1H-pyrrolizin-7a(5H)-yl)methoxy)-4-methoxy-5,6,7,8-tetrahydropyrido[3,4-d]pyrimidine (5.00 g, 1.0 equiv) in DMAC (30.0 mL) was added NaSEt (2.23 g, 3.0 equiv). The reaction was stirred at 60° C. for 1 hour. The mixture was quenched with water (30 mL) and extracted with EtOAc (3×50.0 mL). The combined organic layers were dried over anhydrous sodium sulfate, concentrated, and purified with reversed phase flash chromatography [C18, 0.1% formic acid condition] to afford the title compound (4.50 g, 86% yield) as yellow oil; LCMS (ESI, M+1): m/z=553.1.

Step E. (R)-7-(7-(8-ethyl-7-fluoro-3-(methoxymethoxy)naphthalen-1-yl)-2-(((S,Z)-2-(fluoromethylene)tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione: To a solution of (S,Z)-7-(8-ethyl-7-fluoro-3-(methoxymethoxy)naphthalen-1-yl)-2-((2-(fluoromethylene)tetrahydro-1H-pyrrolizin-7a(5H)-yl)methoxy)-5,6,7,8-tetrahydropyrido[3,4-d]pyrimidin-4-ol (6.00 g, 1.0 equiv) in DMSO (60 mL) were added TEA (3.30 g, 3.0 equiv) and PyBOP (8.48 g, 1.5 equiv). The reaction was stirred at 25° C. for 0.5 hours. Then (R)-1,3,7-triazaspiro[4.5]decane-2,4-dione (2.76 g, 1.5 equiv) was added into the mixture. The reaction was stirred at 25° C. for 12 hours. The mixture was diluted with water (500 mL) and extracted with EtOAc (3×300 mL). The combined organic layers were washed with brine (500 mL), dried over anhydrous sodium sulfate, concentrated, and purified with column chromatography [SiO₂, petroleum ether/ethyl acetate=2/1 to 0/1] to afford the title compound (7.2 g, 90% yield) as red solid; LCMS (ESI, M+1): m/z=704.3.

Step F. (R)-7-(7-(8-ethyl-7-fluoro-3-hydroxynaphthalen-1-yl)-2-(((S,Z)-2-(fluoromethylene)tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione: To a solution of (R)-7-(7-(8-ethyl-7-fluoro-3-(methoxymethoxy)naphthalen-1-yl)-2-(((S,Z)-2-(fluoromethylene)tetrahydro-1H-pyrrolizin-7a(5H)-yl)methoxy)-5,6,7,8-tetrahydropyrido[3,4-d]pyrimidin-4-yl)-1,3,7-triazaspiro[4.5]decane-2,4-dione (1.00 g, 1.0 equiv) in DCM (5 mL) was added TFA (7.68 g, 49.1 equiv). The reaction was stirred at 15° C. for 0.1 hours. The mixture was diluted with DCM (10 mL) and quenched with TEA until pH=8 at −40° C. The mixture was diluted with water (20 mL) and extracted with DCM (3×15 mL). The combined organic layers were washed with brine (20 mL), dried over anhydrous sodium sulfate, concentrated and purified with prep-HPLC [column: Waters Xbridge Prep OBD C18 150×40 mm×10 μm; mobile phase: [water (NH₄HCO₃)-ACN]; gradient: 35%-65% B over 12 min] to afford the title compound (175 mg, 19% yield) as red solid; LCMS (ESI, M+1): m/z=660.3.

### Example A

### KRas Binding Assay

This Example illustrates that exemplary compounds of the present invention bind to KRas and are capable of displacing a labeled tracer ligand occupying the KRas binding site. KRas^{WT}, KRas^{G12A}, KRas^{G12C}, KRas^{G12D}, KRas^{G12R}, KRas^{G12S} KRas^{G12V} KRas^{G13D} or KRas^{Q61H} was used in the assay.

The ability of a compound to bind to KRas was measured using a TR-FRET displacement assay. Biotinylated KRas (corresponding to amino acids 1-169, produced at Accelegan Inc.) was incubated with custom made Cy5 labelled tracer, terbium streptavidin (Cisbio Inc.) and compound (1% DMSO final) in buffer (50 mM HEPES, pH 7.5, 5 mM MgCl₂, 0.005% Tween-20 and 1 mM DTT). After a 60-minute incubation at room temperature, the reaction was measured using a BMG LABTECH CLARIO star Plus via TR-FRET. 100 percent of control (POC) is determined by using a DMSO control and 0 POC is determined using a concentration of control compound that completely inhibits binding of the tracer to KRas. The POC values were fit to a 4-parameter IC₅₀ equation and the IC₅₀ value reported in Table 1 (selected Examples 234-573). The single point inhibition values at 10 nM were reported in Table 2.

TABLE 1
Binding to KRas (IC₅₀ nM) by Exemplary Compounds of Formula (I)

| Example | G12D | G12V | G12R | WT | G13D | Q61H | G12A | G12C | G12S |
|---|---|---|---|---|---|---|---|---|---|
| 234 | <2 | <2 | 4 | | | | | | |
| 235 | 2 | <2 | <2 | | | | | | |
| 236 | 2 | 3 | <2 | | | | | | |
| 237 | 7 | 13 | 19 | | | | | | |
| 238 | 1393 | 1142 | 4612 | | | | | | |
| 239 | 57 | 62 | 21 | | | | | | |
| 240 | 9837 | 2378 | 7482 | | | | | | |
| 241 | 173 | 155 | 48 | | | | | | |
| 242 | 57 | 85 | 49 | | | | | | |
| 243 | 3194 | 422 | 3262 | | | | | | |
| 244 | <2 | <2 | <2 | | | | | | |
| 245 | 13 | 8 | 9 | | | | | | |
| 246 | <2 | <2 | <2 | | | | | | |
| 247 | <2 | <2 | <2 | | | | | | |
| 248 | 1572 | 147 | 515 | | | | | | |
| 250 | 19 | 10 | 9 | | | | | | |
| 251 | 7 | 5 | 3 | | | | | | |
| 252 | 23 | 11 | 7 | | | | | | |
| 253 | <2 | 2 | 3 | | | | | | |
| 254 | 20 | 47 | 50 | | | | | | |
| 255 | 2 | 4 | 4 | | | | | | |
| 256 | <2 | <2 | 2 | | | | | | |
| 257 | <2 | <2 | <2 | | | | | | |
| 258 | <2 | <2 | <2 | | | | | | |
| 259 | <2 | <2 | <2 | | | | | | |
| 260 | <2 | <2 | <2 | | | | | | |
| 261 | <2 | <2 | <2 | | | | | | |
| 262 | <2 | <2 | <2 | | | | | | |
| 263 | 109 | 295 | 261 | | | | | | |
| 264 | <2 | <2 | <2 | | | | | | |
| 265 | <2 | <2 | <2 | | | | | | |
| 266 | 133 | 104 | 254 | | | | | | |
| 267 | 148 | 123 | 256 | | | | | | |
| 268 | <2 | <2 | <2 | | | | | | |
| 269 | <2 | <2 | <2 | | | | | | |
| 270 | <2 | <2 | <2 | | | | | | |
| 271 | <2 | <2 | <2 | | | | | | |
| 272 | 7 | 12 | 15 | | | | | | |
| 273 | <2 | <2 | <2 | | | | | | |
| 274 | 5 | 6 | 10 | | | | | | |
| 275 | <2 | <2 | <2 | | | | | | |
| 276 | <2 | <2 | <2 | | | | | | |
| 277 | <2 | <2 | <2 | | | | | | |
| 278 | <2 | <2 | <2 | | | | | | |
| 279 | 93 | 22 | 25 | | | | | | |
| 280 | 195 | 264 | 230 | | | | | | |
| 281 | 31 | 6 | 11 | | | | | | |
| 282 | <2 | <2 | <2 | | | | | | |
| 283 | <2 | <2 | <2 | | | | | | |
| 284 | <2 | <2 | <2 | | | | | | |
| 285 | 563 | 886 | 773 | | | | | | |
| 286 | <2 | <2 | <2 | | | | | | |
| 287 | 505 | 717 | 441 | | | | | | |
| 288 | 4 | 6 | 9 | | | | | | |
| 289 | 16 | 71 | 57 | | | | | | |
| 290 | 8 | 30 | 26 | | | | | | |
| 291 | 8 | 21 | 20 | | | | | | |
| 292 | <2 | <2 | <2 | | | | | | |
| 293 | <2 | <2 | <2 | | | | | | |
| 294 | <2 | <2 | <2 | | | | | | |
| 295 | <2 | <2 | <2 | | | | | | |
| 296 | <2 | <2 | <2 | | | | | | |
| 297 | 4 | 5 | 7 | | | | | | |
| 298 | 3 | 4 | 5 | | | | | | |
| 299 | <2 | 3 | 3 | | | | | | |
| 300 | <2 | <2 | 3 | | | | | | |
| 301 | <2 | <2 | <2 | | | | | | |
| 302 | 3 | <2 | <2 | | | | | | |
| 303 | <2 | <2 | <2 | | | | | | |
| 304 | 3 | 3 | 3 | | | | | | |
| 305 | 3 | 5 | 5 | | | | | | |
| 306 | <2 | <2 | <2 | | | | | | |
| 307 | <2 | <2 | <2 | | | | | | |
| 308 | <2 | <2 | <2 | | | | | | |
| 309 | 4 | 8 | 7 | | | | | | |
| 310 | <2 | <2 | <2 | | | | | | |
| 311 | <2 | <2 | <2 | | | | | | |
| 312 | <2 | <2 | <2 | | | | | | |
| 313 | 38 | 7 | 25 | | | | | | |
| 314 | 10 | 3 | 7 | | | | | | |
| 315 | 9 | 3 | 5 | | | | | | |
| 316 | 6 | 5 | 6 | | | | | | |
| 317 | 5 | 4 | 4 | | | | | | |
| 318 | <2 | <2 | <2 | | | | | | |
| 319 | 32846 | 4625 | 6885 | | | | | | |
| 320 | <2 | <2 | <2 | | | | | | |
| 321 | 69 | 171 | 146 | | | | | | |
| 322 | <2 | <2 | <2 | | | | | | |
| 323 | <2 | <2 | <2 | | | | | | |
| 324 | 2 | 6 | 4 | | | | | | |
| 325 | <2 | <2 | <2 | | | | | | |
| 326 | <2 | 5 | 4 | | | | | | |
| 327 | <2 | <2 | <2 | | | | | | |
| 328 | 3 | <2 | <2 | | | | | | |
| 329 | <2 | <2 | <2 | | | | | | |
| 330 | <2 | <2 | <2 | | | | | | |
| 331 | <2 | <2 | <2 | | | | | | |
| 332 | 2 | <2 | <2 | | | | | | |
| 333 | <2 | <2 | <2 | | | | | | |
| 334 | 12 | 50 | 43 | | | | | | |
| 335 | 29 | 44 | 32 | | | | | | |
| 336 | 3 | 8 | 7 | | | | | | |
| 337 | <2 | <2 | <2 | | | | | | |
| 338 | 2 | <2 | <2 | | | | | | |
| 339 | 2 | <2 | <2 | | | | | | |
| 340 | <2 | <2 | <2 | | | | | | |
| 341 | 2 | <2 | 2 | | | | | | |
| 342 | <2 | <2 | <2 | | | | | | |
| 343 | 8 | 21 | 18 | | | | | | |
| 344 | <2 | <2 | <2 | | | | | | |
| 345 | 6 | 4 | <2 | | | | | | |
| 346 | 6 | 4 | <2 | | | | | | |
| 347 | 7 | 2 | 2 | | | | | | |
| 348 | 83312 | 38471 | 42979 | | | | | | |
| 349 | <2 | <2 | <2 | | | | | | |
| 350 | <2 | 3 | <2 | | | | | | |
| 351 | 12 | 6 | 4 | | | | | | |
| 352 | <2 | 2 | 2 | | | | | | |
| 353 | <2 | 3 | 4 | | | | | | |
| 354 | 3 | 4 | 5 | | | | | | |
| 355 | 3 | 6 | 9 | | | | | | |
| 356 | <2 | 3 | 3 | | | | | | |
| 357 | 11 | 6 | 7 | | | | | | |
| 358 | <2 | <2 | <2 | | | | | | |
| 359 | 3 | 4 | 2 | | | | | | |
| 360 | <2 | <2 | <2 | | | | | | |
| 361 | 14 | 6 | 5 | | | | | | |
| 362 | <2 | <2 | <2 | | | | | | |
| 363 | <2 | <2 | <2 | | | | | | |
| 364 | 2 | 3 | <2 | | | | | | |
| 365 | <2 | <2 | <2 | | | | | | |
| 366 | <2 | <2 | <2 | | | | | | |
| 367 | <2 | <2 | <2 | | | | | | |
| 368 | 10 | 5 | 6 | | | | | | |
| 369 | <2 | <2 | <2 | | | | | | |
| 370 | <2 | <2 | <2 | | | | | | |
| 371 | 2 | <2 | <2 | | | | | | |
| 372 | <2 | <2 | <2 | | | | | | |
| 373 | <2 | <2 | <2 | | | | | | |
| 374 | <2 | <2 | <2 | | | | | | |
| 375 | <2 | <2 | <2 | | | | | | |
| 376 | 4 | 4 | 3 | | | | | | |
| 377 | 4 | <2 | 4 | | | | | | |
| 378 | <2 | <2 | <2 | | | | | | |
| 379 | <2 | <2 | <2 | | | | | | |
| 380 | <2 | <2 | <2 | | | | | | |
| 381 | <2 | <2 | <2 | | | | | | |
| 382 | <2 | <2 | <2 | | | | | | |
| 383 | <2 | <2 | <2 | | | | | | |
| 384 | <2 | <2 | <2 | | | | | | |
| 385 | <2 | <2 | <2 | | | | | | |
| 386 | <2 | <2 | <2 | | | | | | |
| 387 | <2 | <2 | <2 | | | | | | |
| 388 | <2 | <2 | <2 | | | | | | |
| 389 | <2 | <2 | <2 | | | | | | |
| 390 | <2 | <2 | <2 | | | | | | |
| 391 | <2 | <2 | 3 | | | | | | |
| 392 | 3 | 2 | <2 | | | | | | |
| 393 | 4 | 5 | 4 | | | | | | |
| 394 | 2 | 2 | <2 | | | | | | |
| 395 | <2 | <2 | <2 | | | | | | |
| 396 | <2 | <2 | <2 | | | | | | |
| 397 | <2 | <2 | <2 | | | | | | |
| 398 | <2 | <2 | <2 | | | | | | |
| 399 | 3 | 4 | 8 | | | | | | |
| 400 | <2 | <2 | <2 | | | | | | |
| 401 | 59 | 22 | 10 | | | | | | |
| 402 | <2 | <2 | <2 | | | | | | |
| 403 | <2 | <2 | <2 | | | | | | |
| 404 | 5 | 6 | 3 | | | | | | |
| 405 | 5 | 9 | 3 | | | | | | |
| 406 | <2 | 3 | <2 | | | | | | |
| 407 | 4 | 4 | 2 | | | | | | |
| 408 | <2 | <2 | <2 | | | | | | |
| 409 | <2 | <2 | <2 | | | | | | |
| 410 | 18 | 4 | 7 | | | | | | |
| 411 | <2 | <2 | <2 | | | | | | |
| 412 | <2 | <2 | <2 | | | | | | |
| 413 | <2 | <2 | <2 | | | | | | |
| 414 | 8 | 3 | 11 | | | | | | |
| 415 | <2 | <2 | <2 | | | | | | |
| 416 | 4 | <2 | <2 | | | | | | |
| 417 | 13 | <2 | <2 | | | | | | |
| 418 | 3 | <2 | 2 | | | | | | |
| 419 | <2 | <2 | <2 | | | | | | |
| 420 | <2 | <2 | <2 | | | | | | |
| 421 | 5 | <2 | 3 | | | | | | |
| 422 | 2 | <2 | 3 | | | | | | |
| 423 | 396 | 42 | 26 | | | | | | |
| 424 | 31 | 5 | 9 | | | | | | |
| 425 | <2 | <2 | <2 | | | | | | |
| 426 | <2 | <2 | <2 | | | | | | |
| 427 | 182 | 253 | 537 | | | | | | |
| 429 | <2 | <2 | 3 | | | | | | |
| 430 | <2 | <2 | <2 | | | | | | |
| 431 | 6 | 2 | <2 | | | | | | |
| 432 | 201 | 316 | 592 | | | | | | |
| 433 | 26 | 10 | 11 | | | | | | |
| 434 | 397 | 342 | 498 | 357 | 337 | 547 | 362 | 382 | 405 |
| 435 | 2 | 3 | 2 | | | | | | |
| 436 | 7 | 8 | 17 | 10 | 10 | 10 | 11 | 8 | 10 |
| 437 | <2 | <2 | 5 | 8 | 14 | 12 | 7 | 4 | 4 |
| 438 | <2 | <2 | <2 | <2 | <2 | <2 | <2 | <2 | <2 |
| 439 | 2 | <2 | <2 | <2 | <2 | <2 | <2 | <2 | <2 |
| 440 | 44 | 13 | 36 | 46 | 73 | 63 | 46 | 30 | 41 |
| 441 | 24 | 8 | 14 | 19 | 27 | 21 | 19 | 11 | 15 |
| 442 | <2 | 3 | 3 | 3 | 4 | 2 | <2 | <2 | 3 |
| 443 | 6 | 3 | 4 | 8 | 8 | 5 | 6 | 3 | 4 |
| 444 | 133 | 38 | 40 | 50 | 53 | 87 | 52 | 64 | 42 |
| 445 | 4 | 4 | 8 | 7 | 7 | 9 | 8 | 7 | 3 |
| 446 | 12 | 9 | 3 | | | | | | |
| 447 | <2 | <2 | <2 | | | | | | |
| 448 | 3 | <2 | 2 | <2 | <2 | <2 | <2 | <2 | <2 |
| 449 | 3 | <2 | <2 | <2 | <2 | <2 | <2 | <2 | <2 |
| 450 | 5 | 3 | 3 | 8 | 6 | 5 | 8 | 7 | 7 |
| 451 | 11 | 26 | 62 | 777 | 1155 | 1056 | 387 | 209 | 243 |
| 452 | 4 | 6 | 9 | 7 | 8 | 7 | 9 | 8 | 8 |
| 453 | 604 | 262 | 680 | 826 | 1154 | 1152 | 523 | 464 | 616 |
| 454 | 817 | 333 | 703 | 1077 | 1445 | 1402 | 645 | 585 | 728 |
| 455 | 4 | 2 | <2 | 4 | 4 | 3 | 4 | 3 | 4 |
| 456 | 38 | 18 | 17 | 53 | 59 | 48 | 35 | 32 | 33 |
| 457 | 3557 | 805 | 692 | 1589 | 1873 | 2195 | 1246 | 1913 | 1266 |
| 458 | 48 | 76 | 145 | 143 | 168 | 179 | 148 | 153 | 52 |
| 459 | 16 | 8 | 11 | 10 | 9 | 8 | 11 | 8 | 11 |
| 460 | 67 | 108 | 216 | 137 | 169 | 188 | 142 | 146 | 173 |
| 461 | 11 | 12 | 22 | 23 | 26 | 26 | 22 | 19 | 23 |
| 462 | 16 | 7 | 7 | 9 | 9 | 10 | 10 | 6 | 9 |
| 463 | <2 | <2 | <2 | | | | | | |
| 464 | 9 | 8 | 12 | | | | | | |
| 465 | <2 | <2 | <2 | | | | | | |
| 466 | 83 | 14 | 15 | | | | | | |
| 467 | <2 | <2 | <2 | | | | | | |
| 468 | 2 | 3 | 10 | | | | | | |
| 469 | 8 | 24 | 105 | | | | | | |
| 470 | <2 | <2 | <2 | <2 | <2 | <2 | <2 | <2 | <2 |
| 471 | <2 | <2 | <2 | <2 | <2 | <2 | <2 | <2 | <2 |
| 472 | 7 | 5 | <2 | <2 | 2 | <2 | <2 | 2 | 3 |
| 474 | 79496 | 48547 | 51681 | | | | | | |
| 475 | 6603 | 8631 | 13225 | 11260 | 19089 | 11257 | 8973 | 6987 | 8608 |
| 476 | 5369 | 16873 | 8994 | 13649 | 23939 | 25413 | 8512 | 6598 | 7561 |
| 477 | 2033 | 1675 | 1405 | 4937 | 5615 | 4399 | 2688 | 1497 | 2826 |
| 478 | 24696 | 14498 | 13003 | 12572 | 20002 | 21271 | 12679 | 13141 | 17572 |
| 479 | 99 | 54 | 130 | 33 | 32 | 42 | 55 | 81 | 56 |
| 480 | 229 | 35 | 53 | 52 | 57 | 61 | 58 | 56 | 67 |
| 481 | <2 | 2 | 6 | 11 | 15 | 13 | 7 | 4 | 5 |
| 482 | 3 | <2 | 5 | | | | | | |
| 483 | 9 | 20 | 57 | 150 | 239 | 238 | 81 | 42 | 46 |
| 484 | 3124 | 1177 | 1806 | 2956 | 4434 | 3610 | 2545 | 1538 | 2550 |
| 485 | 369 | 529 | 1224 | | | | | | |
| 486 | 755 | 886 | 1161 | | | | | | |
| 487 | 207 | 244 | 401 | | | | | | |
| 488 | 20 | 9 | 13 | | | | | | |
| 489 | 761 | 702 | 553 | | | | | | |
| 490 | 49 | 9 | 20 | | | | | | |
| 491 | 49 | 3 | <2 | | | | | | |
| 492 | 11 | 7 | 6 | | | | | | |
| 493 | 355 | 510 | 195 | | | | | | |
| 494 | 12 | 26 | 24 | | | | | | |
| 495 | 3 | 12 | 11 | | | | | | |
| 496 | <2 | <2 | <2 | | | | | | |
| 497 | <2 | <2 | 3 | | | | | | |
| 498 | 10 | 9 | 22 | | | | | | |
| 499 | <2 | <2 | <2 | | | | | | |
| 500 | <2 | <2 | <2 | | | | | | |
| 501 | <2 | <2 | 2 | | | | | | |
| 502 | <2 | <2 | 3 | | | | | | |
| 503 | <2 | <2 | <2 | | | | | | |
| 506 | <2 | <2 | <2 | | | | | | |
| 507 | <2 | <2 | <2 | | | | | | |
| 508 | 3 | <2 | <2 | | | | | | |
| 509 | 3 | <2 | <2 | | | | | | |
| 510 | 39 | 47 | 158 | 107 | 89 | 120 | 107 | 80 | 89 |
| 511 | 13 | 15 | 47 | 30 | 28 | 32 | 34 | 20 | 30 |
| 512 | <2 | <2 | 2 | | | | | | |
| 513 | 3 | <2 | <2 | | | | | | |
| 514 | 3218 | 1568 | | 252 | 372 | 389 | 296 | 286 | 318 |
| 515 | 298 | 346 | | 622 | 988 | 877 | 656 | 373 | 725 |
| 516 | 1228 | 1149 | | 2650 | 3179 | 2528 | 1493 | 1027 | 1563 |
| 517 | 1263 | 3098 | | 9325 | 15285 | 11460 | 7188 | 4469 | 6625 |
| 518 | 1926 | 1792 | | 221 | 249 | 190 | 175 | 139 | 161 |
| 519 | 2005 | 1560 | | 5225 | 8138 | 5922 | 5582 | 2854 | 5476 |
| 520 | 2461 | 3986 | | 4611 | 5074 | 5937 | 4154 | 2286 | 2900 |
| 521 | 2724 | 235 | | 432 | 506 | 455 | 438 | 251 | 690 |
| 522 | 3055 | 1847 | | 754 | 1200 | 1016 | 503 | 289 | 281 |
| 523 | 3566 | 491 | | 1908 | 2461 | 2590 | 441 | 385 | 757 |
| 524 | 4254 | 13978 | | 2937 | 3788 | 3025 | 2209 | 1490 | 1901 |
| 525 | 5220 | 4500 | | 20244 | 31571 | 27126 | 25056 | 15019 | 26879 |
| 526 | 5830 | 5809 | 9169 | 111 | 87 | 63 | 175 | 158 | 151 |
| 527 | 6141 | 14052 | | 54995 | 98236 | 65084 | 55406 | 36638 | 53557 |
| 528 | 6289 | 13626 | | 8286 | 7934 | 7524 | 10450 | 13507 | 11680 |
| 529 | 6573 | 3004 | | 2003 | 2108 | 1407 | 1385 | 1199 | 1064 |
| 530 | 6683 | 7534 | | 2656 | 3466 | 3078 | 1920 | 1029 | 1069 |
| 531 | 6832 | 5701 | | 17035 | 24885 | 22404 | 17234 | 9538 | 19938 |
| 532 | 7260 | 756 | | 910 | 1146 | 901 | 649 | 370 | 700 |
| 533 | 7360 | 2676 | | 7621 | 12269 | 9976 | 5969 | 3858 | 4921 |
| 534 | 7366 | 3542 | | 12118 | 18154 | 14633 | 9377 | 4896 | 9329 |
| 535 | 7565 | 4709 | | 5321 | 6192 | 6461 | 2985 | 1831 | 5058 |
| 536 | 7954 | 3023 | | 3053 | 3085 | 2168 | 4686 | 4649 | 7724 |
| 537 | 8518 | 3181 | | 26669 | >100000 | >100000 | 43520 | 3704 | 5661 |
| 538 | 9039 | 1863 | | 3000 | 4358 | 4349 | 2528 | 1330 | 2777 |
| 539 | 9322 | 5876 | | 19924 | 42231 | 41333 | 17141 | 8968 | 20712 |
| 540 | 9349 | 1718 | | 5805 | 8848 | 7733 | 3281 | 1844 | 3752 |
| 541 | 10009 | 5975 | | 256 | 200 | 298 | 259 | 165 | 169 |
| 542 | 11647 | 5368 | | 10434 | 15828 | 18296 | 5738 | 2888 | 5832 |
| 543 | 12103 | 3937 | | 1497 | 2266 | 2398 | 1199 | 634 | 750 |
| 544 | 21082 | 14495 | | 327 | 112 | 121 | 291 | 180 | 204 |
| 545 | 13074 | 6080 | | 7229 | 8092 | 5900 | 4747 | 4779 | 4996 |
| 546 | 13637 | 4638 | | 2641 | 4616 | 3313 | 3803 | 2632 | 3570 |
| 547 | 15871 | 2315 | | 667 | 680 | 784 | 798 | 622 | 707 |
| 548 | 16661 | 5891 | | 10004 | 13941 | 11089 | 6791 | 4119 | 8208 |
| 549 | 17355 | 1981 | | 1906 | 2109 | 2540 | 1669 | 946 | 1358 |
| 550 | 17648 | 8185 | | 32400 | 35313 | 27094 | 33618 | 16316 | 28832 |
| 551 | 18346 | 2524 | | 6325 | 9888 | 10676 | 11694 | 6898 | 11881 |
| 552 | 18425 | 5638 | | 50398 | >100000 | 22850 | 51436 | 26070 | 51010 |
| 553 | 18533 | 2764 | | 5060 | 7558 | 7304 | 2273 | 1582 | 3307 |
| 554 | 18583 | 3831 | | 2753 | 2658 | 1209 | 2422 | 803 | 1749 |
| 555 | 19010 | 6179 | | 12186 | 16444 | 19749 | 11880 | 9191 | 14982 |
| 556 | 19863 | 7056 | | 22868 | 31657 | 44072 | 26560 | 16798 | 26399 |
| 557 | 20811 | 5870 | | 67882 | 96177 | 77086 | 71553 | 44275 | 83249 |
| 558 | 22999 | 2923 | | 978 | 1392 | 1713 | 825 | 492 | 505 |
| 559 | 24671 | 9185 | | 11895 | 45079 | 23261 | 11901 | 11983 | 29435 |
| 560 | 24785 | 3731 | | 6015 | 18358 | 17606 | 6872 | 5464 | 7917 |
| 561 | 25664 | 6113 | | 1282 | 2187 | 1738 | 994 | 557 | 536 |
| 562 | 25915 | 6374 | | 10703 | 21702 | 14002 | 7966 | 4265 | 6601 |
| 563 | 30521 | 6514 | | 776 | 1041 | 231 | 1372 | 697 | 320 |
| 564 | 29513 | 3713 | | 6920 | 7403 | 4382 | 11649 | 8580 | 14216 |
| 565 | 34324 | 6618 | | 12982 | 18928 | 34365 | 12152 | 8615 | 12892 |
| 566 | 35554 | 5703 | | 8781 | 15317 | 15168 | 7987 | 3758 | 8687 |
| 567 | 37648 | 5758 | | 11588 | 11839 | 9553 | 6049 | 3467 | 4334 |
| 568 | 39110 | 5206 | | 15679 | 19786 | 15317 | 13425 | 7898 | 13581 |
| 569 | 41482 | 4611 | | 12163 | 16925 | 10450 | 10802 | 7217 | 8587 |
| 570 | 38996 | 13732 | | 99 | 72 | 37 | 134 | 74 | 58 |
| 571 | 75702 | 6720 | | | | | | | |
| 572 | 89425 | 4560 | | | | | | | |
| 573 | 95966 | 7842 | | | | | | | |
| 574 | <2 | <2 | | <2 | | | | <2 | <2 |
| 575 | 3 | 2 | | <2 | | | | 3 | <2 |
| 576 | 4 | <2 | | 3 | | | | 3 | <2 |
| 577 | <2 | <2 | | <2 | | | | <2 | <2 |
| 578 | <2 | <2 | | <2 | | | | <2 | <2 |
| 579 | 7 | 2 | | 4 | | | | 4 | 3 |
| 580 | <2 | <2 | | <2 | | | | <2 | <2 |
| 581 | 10 | 5 | | 6 | | | | 6 | 4 |
| 582 | 3 | <2 | | <2 | | | | 2 | 2 |
| 583 | 3 | <2 | | <2 | | | | <2 | <2 |
| 585 | 3 | <2 | <2 | | | | | | |
| 586 | <2 | <2 | <2 | | | | | | |
| 587 | <2 | <2 | <2 | | | | | | |
| 588 | <2 | <2 | <2 | | | | | | |
| 589 | <2 | <2 | <2 | | | | | | |
| 590 | 4 | 2 | <2 | | | | | | |
| 591 | 37720 | 37588 | >100000 | >100000 | >100000 | >100000 | >100000 | >100000 | >100000 |
| 592 | <2 | <2 | <2 | | | | | | |
| 593 | <2 | <2 | <2 | | | | | | |
| 594 | 13 | 2 | <2 | | | | | | |
| 595 | 2 | <2 | <2 | | | | | | |
| 596 | 11 | 7 | 9 | | | | | | |
| 597 | 14 | 3 | <2 | | | | | | |
| 598 | 16 | 12 | 8 | | | | | | |
| 599 | 7 | 4 | 3 | | | | | | |
| 600 | 33 | 25 | 7 | | | | | | |
| 601 | 11 | 8 | 14 | | | | | | |
| 602 | 44 | 6 | 3 | | | | | | |
| 603 | 30 | 7 | 8 | | | | | | |
| 604 | 9 | 12 | 15 | | | | | | |
| 605 | 7 | 5 | 11 | | | | | | |
| 606 | 16 | 16 | 36 | | | | | | |
| 607 | <2 | <2 | <2 | | | | | | |
| 608 | <2 | <2 | <2 | | | | | | |
| 609 | <2 | <2 | <2 | | | | | | |
| 610 | <2 | <2 | <2 | | | | | | |
| 611 | <2 | <2 | <2 | | | | | | |
| 612 | <2 | <2 | <2 | 8 | <2 | 10 | <2 | <2 | <2 |
| 613 | <2 | <2 | <2 | | | | | | |
| 614 | <2 | <2 | <2 | | | | | | |
| 615 | 3 | <2 | 2 | | | | | | |
| 616 | 4 | <2 | 3 | | | | | | |
| 619 | <2 | <2 | <2 | | | | | | |
| 620 | <2 | <2 | <2 | | | | | | |
| 623 | <2 | <2 | <2 | | | | | | |
| 624 | <2 | <2 | <2 | | | | | | |
| 625 | 12161 | 12784 | 25916 | 12876 | 11279 | 17814 | 12982 | 16472 | 15505 |
| 626 | 6472 | 3029 | 2297 | 4066 | 3958 | 5209 | 5414 | 4736 | 4936 |
| 627 | 42 | 23 | 23 | | | | | | |
| 628 | <2 | <2 | <2 | | | | | | |
| 629 | 35 | 10 | 51 | | | | | | |
| 630 | 4 | 8 | 6 | | | | | | |
| 631 | <2 | <2 | <2 | | | | | | |
| 632 | <2 | <2 | <2 | | | | | | |
| 633 | <2 | <2 | <2 | | | | | | |
| 634 | <2 | <2 | <2 | | | | | | |
| 635 | <2 | <2 | <2 | | | | | | |
| 636 | <2 | <2 | <2 | | | | | | |
| 637 | <2 | <2 | <2 | | | | | | |
| 638 | 2949 | 5120 | 10850 | 5197 | 4069 | 5380 | 4762 | 4503 | 5892 |
| 639 | 241 | 357 | 1279 | 719 | 566 | 900 | 619 | 661 | 571 |
| 640 | 50037 | 17942 | 15478 | 25718 | 25569 | 28242 | 21906 | 29089 | 19390 |
| 641 | <2 | 2 | <2 | | | | | | |
| 642 | <2 | <2 | 3 | | | | | | |
| 643 | <2 | <2 | <2 | | | | | | |
| 644 | 16 | 14 | 13 | | | | | | |
| 645 | 52 | 13 | 7 | | | | | | |
| 646 | 95 | 23 | 20 | | | | | | |
| 647 | 5574 | 1137 | 392 | | | | | | |
| 648 | 17 | 7 | 4 | | | | | | |
| 649 | 124 | 29 | 34 | | | | | | |
| 650 | 3439 | 718 | 528 | | | | | | |
| 651 | 18 | <2 | <2 | | | | | | |
| 652 | 3120 | 1238 | 1412 | | | | | | |
| 653 | <2 | <2 | | <2 | | | | <2 | <2 |
| 654 | 2 | <2 | 3 | | | | | | |
| 655 | 5 | 4 | | 5 | | | | 4 | 5 |
| 656 | <2 | <2 | | 5 | | | | 2 | 2 |
| 657 | <2 | 12 | | 5 | | | | 7 | 4 |
| 658 | <2 | <2 | | <2 | | | | <2 | <2 |
| 659 | <2 | <2 | | <2 | | | | <2 | <2 |
| 660 | 3 | <2 | | <2 | | | | <2 | <2 |
| 661 | 143 | 33 | | 25 | | | | 32 | 21 |
| 662 | 14353 | 4041 | | 10252 | 13581 | 10459 | 9522 | 5471 | 10078 |
| 663 | 2313 | 1421 | | | | | | | |
| 664 | 254 | 317 | | | | | | | |
| 665 | 6838 | 10765 | | 87608 | >100000 | >100000 | 83138 | 37778 | 62776 |
| 666 | 340 | 238 | | | | | | | |
| 667 | 223 | 243 | | | | | | | |
| 668 | 8990 | 2054 | | | | | | | |
| 669 | 190 | 199 | | | | | | | |
| 670 | 9752 | 1539 | | 1958 | 1974 | 2032 | 1876 | 602 | 1978 |
| 671 | 11810 | 1980 | | 23890 | 32955 | 30152 | 10195 | 5239 | 16487 |
| 672 | 1282 | 1440 | | | | | | | |
| 673 | 8425 | 2440 | | | | | | | |
| 674 | 1413 | 1247 | | 3488 | 4054 | 3962 | 3968 | 2029 | 3697 |
| 675 | 6300 | 3242 | | 16863 | 15315 | 25254 | 17256 | 8482 | 16566 |
| 676 | 4293 | 1914 | | 5005 | 6223 | 6700 | 6300 | 3412 | 7089 |
| 677 | 2327 | 4409 | | 11010 | 13931 | 14139 | 9113 | 6330 | 13558 |
| 678 | 197 | 210 | | | | | | | |
| 679 | 5891 | 3188 | | | | | | | |
| 680 | 5486 | 3130 | | | | | | | |
| 681 | 3834 | 3218 | | | | | | | |
| 682 | 1605 | 1755 | | | | | | | |
| 683 | 8311 | 5845 | | 10833 | 16061 | 12074 | 8280 | 5450 | 8599 |
| 684 | 41671 | 11244 | 11364 | 1876 | 2219 | 2210 | 2642 | 1810 | 1957 |
| 685 | 1649 | 1863 | | | | | | | |
| 686 | 5251 | 4525 | | | | | | | |
| 687 | 10819 | 6228 | | | | | | | |
| 688 | 37395 | 6430 | | 14587 | 14474 | 18070 | 9730 | 10009 | 17688 |
| 689 | 7422 | 4309 | | | | | | | |
| 690 | 5443 | 1361 | | | | | | | |
| 691 | 7297 | 5297 | | | | | | | |
| 692 | 16068 | 8852 | | | | | | | |
| 693 | 3295 | 2398 | | | | | | | |
| 694 | 131 | 437 | | 1933 | 4475 | 4853 | 2219 | 651 | 946 |
| 695 | 3908 | 3988 | | | | | | | |
| 696 | 5164 | 1958 | | | | | | | |
| 697 | 3785 | 1457 | | | | | | | |
| 698 | 6020 | 4043 | | | | | | | |
| 699 | 6149 | 5462 | | 7339 | 9977 | 8741 | 8445 | 4201 | 9984 |
| 700 | 1379 | 452 | 687 | 522 | 374 | 577 | 474 | 251 | 399 |
| 701 | 7735 | 2026 | | 5292 | 6622 | 6069 | 6298 | 3618 | 5832 |
| 702 | 2029 | 1117 | | | | | | | |
| 703 | 10737 | 4792 | | 12388 | 16078 | 15882 | 13534 | 9490 | 13503 |
| 704 | 3222 | 3075 | | | | | | | |
| 705 | 3068 | 8524 | | 13285 | 21835 | 15899 | 16475 | 10932 | 20520 |
| 706 | 7672 | 2204 | | 3312 | 3334 | 3769 | 2805 | 1685 | 2820 |
| 707 | 6275 | 3315 | | | | | | | |
| 708 | 2262 | 2473 | | | | | | | |
| 709 | 17 | 31 | | 95 | | | | 72 | 83 |
| 710 | 57 | 60 | | 52 | | | | 48 | 60 |
| 711 | 17 | 21 | | 10 | | | | 19 | 13 |
| 712 | 3 | 3 | | 2 | | | | 2 | <2 |
| 713 | 2 | <2 | | <2 | | | | <2 | <2 |

TABLE 2
Binding to KRas (% inhibition at 10 nM)
by Exemplary Compounds of Formula (I)

| Ex. No. | G12A | G13D | G12C | Q61H | G12S | WT |
|---|---|---|---|---|---|---|
| 236 | 70 | 65 | 87 | 67 | 81 | 66 |
| 257 | 97 | 98 | 99 | 100 | 99 | 98 |
| 258 | 98 | 98 | 100 | 97 | 99 | 97 |
| 259 | 94 | 96 | 97 | 96 | 98 | 94 |
| 260 | 97 | 98 | 99 | 98 | 99 | 98 |
| 261 | 98 | 97 | 99 | 96 | 99 | 97 |
| 262 | 95 | 97 | 99 | 96 | 98 | 95 |
| 264 | 95 | 97 | 100 | 98 | 99 | 96 |
| 265 | 97 | 99 | 100 | 97 | 99 | 98 |
| 268 | 98 | 99 | 99 | 99 | 99 | 98 |
| 269 | 99 | 99 | 100 | 98 | 100 | 99 |
| 282 | 98 | 99 | 99 | 99 | 100 | 98 |
| 283 | 97 | 98 | 99 | 97 | 99 | 95 |
| 284 | 88 | 90 | 95 | 89 | 96 | 88 |
| 286 | 94 | 94 | 98 | 95 | 97 | 94 |
| 288 | 38 | 40 | 56 | 33 | 53 | 38 |
| 292 | 98 | 98 | 100 | 100 | 99 | 99 |
| 293 | 95 | 91 | 99 | 92 | 99 | 92 |
| 294 | 98 | 98 | 100 | 98 | 100 | 98 |
| 295 | 99 | 98 | 99 | 98 | 99 | 99 |
| 296 | 94 | 93 | 98 | 93 | 98 | 93 |
| 301 | 89 | 91 | 99 | 94 | 97 | 89 |
| 302 | 85 | 91 | 96 | 93 | 93 | 89 |
| 303 | 98 | 98 | 100 | 98 | 99 | 97 |
| 304 | 63 | 72 | 83 | 73 | 77 | 70 |
| 306 | 96 | 97 | 99 | 96 | 99 | 95 |
| 307 | 94 | 94 | 98 | 96 | 97 | 95 |
| 308 | 95 | 93 | 98 | 94 | 98 | 94 |
| 310 | 98 | 98 | 100 | 98 | 99 | 97 |
| 311 | 98 | 98 | 100 | 98 | 100 | 98 |
| 312 | 94 | 92 | 98 | 93 | 97 | 93 |
| 318 | 0 | 0 | 0 | 1 | 0 | 0 |
| 322 | 97 | 97 | 99 | 98 | 99 | 97 |
| 323 | 97 | 98 | 99 | 99 | 99 | 98 |
| 325 | 96 | 96 | 99 | 96 | 99 | 95 |
| 326 | 61 | 87 | 77 | 86 | 81 | 84 |
| 327 | 83 | 96 | 99 | 96 | 96 | 92 |
| 328 | 87 | 91 | 96 | 91 | 95 | 88 |
| 329 | 92 | 93 | 98 | 94 | 98 | 93 |
| 330 | 87 | 91 | 97 | 91 | 96 | 91 |
| 331 | 97 | 97 | 99 | 97 | 99 | 97 |
| 332 | 94 | 93 | 98 | 95 | 97 | 93 |
| 333 | 98 | 98 | 99 | 98 | 99 | 98 |
| 337 | 97 | 96 | 99 | 98 | 99 | 97 |
| 338 | 90 | 92 | 97 | 93 | 97 | 92 |
| 339 | 92 | 92 | 98 | 94 | 97 | 93 |
| 340 | 97 | 94 | 98 | 97 | 99 | 94 |
| 341 | 87 | 92 | 97 | 95 | 96 | 93 |
| 342 | 96 | 96 | 99 | 96 | 99 | 96 |
| 344 | 97 | 96 | 99 | 97 | 99 | 97 |
| 349 | 96 | 96 | 99 | 98 | 99 | 96 |
| 358 | 93 | 94 | 99 | 95 | 99 | 94 |
| 359 | 65 | 68 | 85 | 62 | 79 | 62 |
| 360 | 98 | 97 | 100 | 97 | 99 | 97 |
| 362 | 97 | 95 | 99 | 95 | 99 | 96 |
| 363 | 93 | 89 | 98 | 92 | 95 | 91 |
| 365 | 97 | 97 | 99 | 97 | 99 | 97 |
| 366 | 95 | 94 | 99 | 95 | 98 | 94 |
| 367 | 99 | 98 | 100 | 99 | 99 | 98 |
| 370 | 97 | 98 | 99 | 98 | 99 | 97 |
| 371 | 91 | 87 | 98 | 89 | 96 | 86 |
| 372 | 99 | 98 | 99 | 99 | 99 | 98 |
| 373 | 96 | 96 | 99 | 97 | 99 | 96 |
| 374 | 96 | 94 | 99 | 96 | 99 | 95 |
| 375 | 94 | 93 | 98 | 93 | 98 | 93 |
| 376 | 54 | 61 | 78 | 51 | 70 | 53 |
| 378 | 90 | 90 | 97 | 91 | 96 | 91 |
| 379 | 96 | 92 | 99 | 92 | 98 | 93 |
| 380 | 96 | 94 | 99 | 95 | 99 | 93 |
| 381 | 97 | 97 | 99 | 97 | 99 | 97 |
| 382 | 98 | 98 | 100 | 97 | 99 | 97 |
| 383 | 96 | 97 | 99 | 98 | 99 | 97 |
| 384 | 91 | 93 | 99 | 95 | 98 | 94 |
| 385 | 96 | 94 | 98 | 96 | 98 | 95 |
| 386 | 92 | 96 | 99 | 97 | 98 | 95 |
| 387 | 99 | 99 | 100 | 99 | 100 | 99 |
| 388 | 94 | 98 | 100 | 99 | 99 | 98 |
| 390 | 95 | 95 | 99 | 97 | 98 | 95 |
| 391 | 81 | 86 | 93 | 85 | 94 | 81 |
| 392 | 56 | 50 | 78 | 54 | 71 | 55 |
| 393 | 66 | 64 | 83 | 60 | 72 | 60 |
| 394 | 71 | 70 | 88 | 63 | 80 | 65 |
| 396 | 98 | 97 | 99 | 99 | 100 | 98 |
| 397 | 95 | 94 | 99 | 97 | 98 | 95 |
| 398 | 92 | 96 | 99 | 98 | 98 | 96 |
| 400 | 96 | 95 | 99 | 96 | 99 | 96 |
| 402 | 98 | 98 | 100 | 98 | 99 | 98 |
| 403 | 100 | 100 | 100 | 100 | 100 | 100 |
| 406 | 73 | 70 | 87 | 66 | 82 | 68 |
| 407 | 61 | 56 | 78 | 56 | 71 | 56 |
| 408 | 98 | 97 | 99 | 97 | 100 | 98 |
| 409 | 100 | 100 | 100 | 100 | 100 | 100 |
| 411 | 74 | 75 | 90 | 71 | 88 | 73 |
| 412 | 62 | 60 | 83 | 61 | 76 | 62 |
| 413 | 98 | 99 | 100 | 100 | 99 | 99 |
| 415 | 100 | 99 | 100 | 100 | 100 | 99 |
| 419 | 54 | 82 | 83 | 90 | 72 | 62 |
| 420 | 96 | 95 | 98 | 97 | 97 | 95 |
| 422 | 55 | 55 | 72 | 62 | 60 | 58 |
| 465 | 77 | 96 | 97 | 96 | 93 | 92 |
| 470 | 97 | 99 | 98 | 99 | 99 | 99 |

### Example B

Inhibition of KRas Phosphorylation of ERK (HTRF) by Exemplary Compounds of Formula (I) Cisbio HTRF Advanced pERK Assay Catalog #64AERPEH
Cells: MKN1, PSN1
Procedure:
   Day 1: Seed 6,000 cells/well -25 pl/well in 384-well white solid bottom plate; RPM1_10% FBS. Incubate overnight at 37° C./5% CO₂.
   Day 2: Echo transfer 25 nl of 10 mM compound 10 point dilution at 1:3 (Cf=10 uM) and incubate for 3 hour at 37° C./5% CO₂.
   Add 8.5 μl/well of 4X Lysis Buffer/25X Blocking reagent (do not dump media) and incubate for 30 min at room temperature on shaker.
   Add conjugate mixture of 4.25 μl/well 1X-pERK-D2 and 1X-pERK-K diluted in Detection Buffer for a total of 8.5 μl/well.
   Incubate for 4 hours at room temperature covered.
   Read HTRF using ClarioStar
>Cells: ASPC1, H727, A549, H460, HCT116, H358, H2009
Culture/Assay media: RPMI-1640+10% FBS
Procedure:

### Cell Seeding

1. To harvest cells from flask using 0.05% Trypsin/EDTA solution. Add 10 mL of media to stop trypsinizing. Pipette the cells into a conical bottom 50 mL centrifuge tube and centrifuge 5 min×1000 rpm.
2. Re-suspend the cell pellet in media, take a cell count, and then adjust the cell density using fresh media.
3. Seed 6,000 cells into cell culture plate with 50 μL media. The
4. Incubate cell plate overnight in a 37° C., 5% CO₂ incubator.

### Compound Titrations

1. Use Tecan to complete the compound addition. Compounds start from 10 uM top, 3-fold dilution, and 10 doses. The final DMSO concentration is 0.8%. Dispensed 0.2 uM Trametinib as Min control.

### 2. Incubate Cell Plate for 3 Hrs in the Incubator.

Detection with cisbio pERK HTRF kit
1. Dilute 1 volume of 4x lysis buffer with 3 volumes of deionized water. Then, add 100X the blocking reagent. Keep lysis buffer on the ice.
2. At the end of the compound treatment, flick-off the media.
3. Add 35 μL of lysis buffer per well using a Multidrop Combi. Then place on a plate agitator shaking at 300 rpm at 4° C. for 40 mins.
4. Make up the HTRF antibody buffer. For each assay plate, mix 50 μL of d₂-conjugate antibody with 950 μL of detection buffer. Similarly, mix 50 μL of Cryptate antibody with 950 μL of detection buffer. Then mix the two diluted antibodies together.
5. Dispense 3.4 μL the antibody buffer to wells of an empty assay plate. Seal the plate and centrifuge plate 30 sec x 1000 rpm.
6. At the end of the 4° C. lysis, centrifuge the lysate plates 3 mins×1500 rpm.
7. Use the Bravo to transfer 13.6 μL of lysate from cell culture plate to assay plate. Then incubate assay plate for 2 hrs at room temperature.
8. At the end of incubation, read plate on the Envision after centrifuging plate 30 sec x 1000 rpm.

pERK IC_{50S} were reported in Table 3 and single point % inhibitions at 100 nM were reported in Table 4.

TABLE 3
Inhibition (HTRF IC₅₀ nM) of KRas -mediated Phosphorylation
of ERK by Exemplary Compounds of Formula (I)

| Example | AsPC-1 | H727 | MKN1 | HCT116 | A549 | H460 | H358 | H2009 | PSN1 |
|---|---|---|---|---|---|---|---|---|---|
| 233 | 94 | 346 | | | | | | | |
| 234 | 52 | 1564 | | | | | | | |
| 235 | 52 | 302 | 294.5 | | | | | | >10000 |
| 236 | 65 | 72 | 14.4 | 346 | 68 | 137 | | | >10000 |
| 237 | 639 | 1637 | | | | | | | |
| 239 | 3137 | 4098 | | | | | | | |
| 241 | 7112 | 5019 | | | | | | | |
| 242 | 3482 | 5632 | | | | | | | |
| 244 | 28 | 104 | 17 | | | | | | >10000 |
| 245 | 1455 | 1640 | | | | | | | |
| 246 | 26 | 124 | 54 | 442 | 200 | 301 | 45 | | >10000 |
| 247 | 7 | 10 | 22.3 | 110 | 15 | 33 | 5 | | 7613 |
| 249 | 161 | 124 | | | | | | | |
| 250 | 283 | 763 | | | | | | | |
| 251 | 1312 | 1204 | | | | | | | |
| 252 | 1953 | 1737 | | | | | | | |
| 253 | 1310 | 3047 | | | | | | | |
| 254 | 871 | 1364 | | | | | | | |
| 255 | 565 | 563 | | | | | | | |
| 256 | 443 | 508 | | | | | | | |
| 257 | 31 | 55 | 1.2 | | | | | | >10000 |
| 258 | 29 | 24 | 5.4 | | | | | | 8638 |
| 259 | 32 | 287 | 3.3 | | | | | | 3936 |
| 260 | 2 | 8 | 29.2 | 151 | 53 | 102 | 17 | | >10000 |
| 261 | 23 | 63 | 57.4 | | | | | | >10000 |
| 262 | 14 | 78 | 56 | | | | | | >10000 |
| 263 | 5374 | 8771 | | | | | | | |
| 264 | 51 | 259 | | | | | | | |
| 265 | 31 | 217 | 23.1 | | | | | | >10000 |
| 266 | 787 | 1755 | | | | | | | |
| 267 | 3618 | 3953 | | | | | | | |
| 268 | 18 | 32 | 8.9 | 218 | 83 | 56 | 7 | | >10000 |
| 269 | 3 | 5 | 1.4 | 25 | 2 | 8 | 1 | | 9883 |
| 270 | 20 | 106 | 13.8 | | | | | | >10000 |
| 271 | 10 | 18 | 42.9 | | | | | | >10000 |
| 272 | 528 | 1929 | 509.7 | | | | | | >10000 |
| 273 | 202 | 239 | 218.1 | | | | | | >10000 |
| 274 | 697 | 1149 | | | | | | | |
| 275 | 26 | 115 | 91.3 | | | | | | >10000 |
| 276 | 37 | 297 | 144.4 | | | | | | >10000 |
| 277 | 37 | 210 | 41.7 | | | | | | >10000 |
| 278 | 68 | 659 | 155.8 | | | | | | >10000 |
| 279 | >10000 | 5078 | | | | | | | |
| 280 | 7001 | >10000 | | | | | | | |
| 281 | >10000 | 4440 | | | | | | | |
| 282 | 9 | 128 | 35.1 | 172 | 53 | 93 | 16 | | >10000 |
| 283 | 22 | 106 | 155.2 | | | | | | >10000 |
| 284 | 3 | 21 | 30.5 | 157 | 22 | 64 | 10 | | >10000 |
| 285 | >10000 | >10000 | | | | | | | |
| 286 | 4 | 8 | 11.9 | 211 | 29 | 49 | 6 | | 3270 |
| 287 | 6447 | >10000 | | | | | | | |
| 288 | 67 | 189 | | | | | | | |
| 289 | 462 | 1882 | | | | | | | |
| 290 | 476 | 966 | | | | | | | |
| 29 | 127 | 336 | | | | | | | |
| 292 | 2 | 5 | 2 | 40 | 8 | 17 | 1 | | 2234 |
| 293 | 3 | 8 | 15.9 | 224 | 18 | 48 | 3 | | 6012 |
| 294 | 4 | 10 | 4 | 41 | 7 | 15 | 2 | | 3798 |
| 295 | 20 | 23 | 3.5 | | | | | | >10000 |
| 296 | 4 | 20 | 8.9 | 118 | 12 | 38 | 5 | | 6582 |
| 297 | 142 | 259 | | | | | | | |
| 299 | 574 | 996 | | | | | | | |
| 300 | 107 | 173 | | | | | | | |
| 301 | 12 | 6 | 12.9 | | | | | | 655 |
| 302 | 9 | 20 | 26.3 | 167 | 27 | 49 | 6 | | 9490 |
| 303 | 2 | 36 | 22.5 | 164 | 35 | 73 | 8 | | 6700 |
| 304 | 54 | 180 | | | | | | | |
| 305 | 441 | 506 | | | | | | | |
| 306 | 3 | 5 | 7 | 58 | 16 | 23 | 1 | | 2035 |
| 307 | 5 | 9 | 9.1 | 179 | 18 | 42 | 5 | | 2968 |
| 308 | 13 | 13 | 11 | | | | | | 3485 |
| 309 | 484 | 773 | | | | | | | |
| 310 | 9 | 47 | 44.6 | 171 | 40 | 101 | 12 | | >10000 |
| 311 | 4 | 5 | 3.7 | 43 | 11 | 23 | 1 | | 1810 |
| 312 | 9 | 24 | 4.3 | 116 | 19 | 26 | 4 | | 8655 |
| 313 | 2401 | 747 | | | | | | | |
| 314 | 803 | 406 | | | | | | | |
| 315 | 1305 | 681 | | | | | | | |
| 316 | 879 | 687 | | 3136 | 1389 | 1767 | 536 | | |
| 317 | 185 | 327 | 63 | | | | | | >10000 |
| 318 | 3 | 17 | 2.6 | 21 | 6 | 13 | 1 | | 4657 |
| 319 | 7076 | 3356 | | | | | | | |
| 320 | 5 | 6 | 4.2 | 214 | 20 | 41 | 5 | | 1417 |
| 321 | 1896 | 1916 | | | | | | | |
| 322 | 1 | 2 | 3.1 | 80 | 12 | 23 | 2 | | 1305 |
| 323 | 2 | 2 | 4.4 | 52 | 14 | 22 | 1 | | 1564 |
| 324 | 830 | 1175 | | | | | | | |
| 325 | 2 | 2 | 7.8 | 71 | 17 | 36 | 2 | | 2225 |
| 326 | 78 | 296 | 14.7 | | | | | | >10000 |
| 327 | 3 | 3 | 6.6 | 114 | 17 | 56 | 3 | | 3614 |
| 328 | 11 | 14 | 23.5 | | | | | | 5574 |
| 329 | 5 | 8 | 7.8 | | | | | | 4153 |
| 330 | 7 | 16 | 14.5 | 117 | 8 | 30 | 8 | | 6082 |
| 331 | 2 | 2 | 4 | 42 | 8 | 16 | 1 | | 2052 |
| 332 | 5 | 9 | 10.4 | 221 | 28 | 43 | 6 | | 6244 |
| 333 | 2 | 8 | 2.6 | 32 | 8 | 10 | 2 | | 3657 |
| 334 | 999 | 1686 | | | | | | | |
| 335 | 779 | 656 | | | | | | | |
| 336 | 289 | 517 | 530.6 | | | | | | >10000 |
| 337 | 2 | 3 | 2.9 | 69 | 14 | 22 | 2 | | 3897 |
| 338 | 6 | 11 | 9.6 | 226 | 21 | 67 | 7 | | 5433 |
| 339 | 6 | 6 | 8.4 | 156 | 17 | 43 | 4 | | 5717 |
| 340 | 8 | 4 | 7.5 | 110 | 11 | 31 | 3 | | 2507 |
| 341 | 8 | 9 | 6.7 | 141 | 19 | 42 | 3 | | 4530 |
| 342 | 2 | 4 | 3.9 | 74 | 8 | 23 | 2 | | 2395 |
| 343 | 445 | 335 | | | | | | | |
| 344 | 2 | 3 | 4.1 | 55 | 7 | 24 | 2 | | 2031 |
| 345 | 1641 | | | | | | | | |
| 346 | 1224 | 822 | | | | | | | |
| 347 | 690 | 541 | | | | | | | |
| 348 | >10000 | >10000 | | | | | | | |
| 349 | 8 | 2 | 3.9 | 74 | 6 | 24 | 2 | | 862 |
| 350 | 105 | 178 | 10.9 | | | | | | >10000 |
| 351 | 3657 | 1059 | | | | | | | |
| 352 | 1823 | 1948 | 1105.6 | | | | | | >10000 |
| 353 | 400 | 561 | 516.4 | | | | | | >10000 |
| 354 | 1063 | 1218 | 814.2 | | | | | | >10000 |
| 355 | 597 | 562 | 417 | | | | | | >10000 |
| 356 | 262 | 284 | 216.5 | | 524 | | | | >10000 |
| 357 | 4411 | 1834 | 2598.2 | | | | | | >10000 |
| 358 | 4 | 16 | 20.6 | 111 | 10 | 25 | 4 | | 7405 |
| 359 | 50 | 122 | 133.6 | | 75 | | | | >10000 |
| 360 | 9 | 4 | 12 | 67 | 9 | 20 | 2 | | 1372 |
| 361 | 636 | 466 | 400.7 | | | | | | >10000 |
| 362 | 5 | 11 | 11.2 | 172 | 20 | 56 | 7 | | 2644 |
| 363 | 16 | 12 | 15.3 | | 16 | | | | 5811 |
| 364 | 997 | 716 | 455.6 | | | | | | >10000 |
| 365 | 4 | 9 | 3.4 | 94 | 25 | 41 | 6 | | 3330 |
| 366 | 4 | 4 | 4.6 | 92 | 19 | 35 | 4 | | 4634 |
| 367 | 2 | 3 | 4.2 | 40 | 5 | 24 | 1 | | 2097 |
| 368 | 666 | 489 | 69.1 | | | | | | 7047 |
| 369 | 995 | 162 | 3.6 | | | | | | 3256 |
| 370 | 9 | 20 | 6.9 | 132 | 36 | 43 | 6 | | >10000 |
| 371 | 3 | 5 | 9.9 | 187 | 9 | 35 | 3 | | 5084 |
| 372 | 2 | 5 | 3.1 | 54 | 10 | 11 | 2 | | 2009 |
| 373 | 2 | 4 | 6.6 | 48 | 11 | 14 | 2 | | 4154 |
| 374 | 4 | 6 | 13.5 | 105 | 25 | 35 | 5 | | 4822 |
| 375 | 4 | 7 | 16.6 | 91 | 20 | 36 | 4 | | 3499 |
| 376 | 55 | 124 | 76.1 | | | 112 | | | >10000 |
| 377 | 2338 | 677 | | | | | | | |
| 378 | 3 | 7 | 26.2 | 160 | 39 | 46 | 9 | | 5171 |
| 379 | 2 | 3 | 12.3 | 159 | 17 | 33 | 3 | | 5327 |
| 380 | 2 | 5 | 10.8 | 95 | 2 | 30 | 3 | | 4719 |
| 381 | 2 | 3 | 7 | 45 | 7 | 11 | 1 | | 3618 |
| 382 | 2 | 4 | 8.4 | 51 | 15 | 25 | 1 | | 2290 |
| 383 | 1 | 2 | 7.8 | 73 | 5 | 13 | 2 | | 3679 |
| 384 | 2 | 6 | 9 | 91 | 17 | 25 | 5 | | 3878 |
| 385 | 3 | 5 | 14.4 | 85 | 15 | 22 | 2 | | 3984 |
| 386 | 1 | 2 | 11.3 | 39 | 4 | 19 | 1 | | 2712 |
| 387 | 1 | 2 | 3 | 14 | 1 | 7 | 1 | | 1796 |
| 388 | 1 | 2 | 8.1 | 42 | 9 | 20 | 1 | | 2348 |
| 389 | 492 | 214 | 54.8 | >10000 | | | | | >10000 |
| 390 | 1 | 3 | 9.6 | 42 | 5 | 15 | 1 | | 3846 |
| 391 | 4 | 26 | 57.6 | 128 | | 43 | | | >10000 |
| 392 | 72 | 136 | 111.3 | 813 | | 285 | | | >10000 |
| 393 | 59 | 201 | 58.5 | 235 | | 72 | | | >10000 |
| 394 | 28 | 109 | 49.4 | 257 | | 79 | | | >10000 |
| 395 | 168 | 392 | 17.5 | 403 | | 169 | | | 606 |
| 396 | 57 | 68 | 78.9 | 551 | 206 | 273 | | | 2571 |
| 397 | 1 | 2 | 11.4 | 42 | 6 | 21 | | | 3258 |
| 398 | 2 | 3 | 7 | 35 | 2 | 15 | | | 1570 |
| 399 | 513 | 896 | | | | | | | |
| 400 | 2 | 5 | 13.5 | 94 | 8 | 32 | | | 3790 |
| 401 | 1728 | 1406 | | | | | | | |
| 402 | 2 | 10 | 5.7 | 54 | 23 | 24 | | | 3907 |
| 403 | 2 | 7 | 2 | 44 | 3 | 18 | | | 2945 |
| 404 | 182 | 188 | 43.5 | 424 | 134 | 178 | | | >10000 |
| 405 | 339 | 308 | 99.9 | | 273 | | | | >10000 |
| 406 | 59 | 100 | 24.6 | 331 | 110 | 143 | | | >10000 |
| 407 | 97 | 86 | 32.9 | 593 | 124 | 151 | | | >10000 |
| 408 | 90 | 166 | 21.7 | 1035 | 363 | 594 | | | 5512 |
| 409 | 8 | 7 | 3.1 | 212 | 22 | 57 | | | 1218 |
| 410 | >10000 | 1673 | 216.7 | | | | | | >10000 |
| 411 | 31 | 54 | 30.1 | 423 | 111 | 260 | | | 6542 |
| 412 | 96 | 72 | 1.7 | 614 | 213 | 207 | | | >10000 |
| 413 | 1 | 3 | 0.4 | 23 | 4 | 5 | 1 | | 2183 |
| 414 | 2999 | 935 | 28.4 | | | | | | 7538 |
| 415 | 26 | 28 | 16 | 504 | 134 | 171 | | | 1982 |
| 416 | 486 | 346 | 19.9 | | 100 | | | | 4709 |
| 417 | 208 | 128 | 29.3 | 273 | 63 | 84 | | | 5703 |
| 418 | 117 | 193 | | 787 | 236 | 375 | | | |
| 419 | 40 | 37 | 6 | 38 | 9 | 20 | | | 194 |
| 420 | 66 | 38 | 5.8 | 143 | 24 | 38 | | | 2743 |
| 421 | 377 | 275 | 448.8 | 3261 | 322 | | | | 9626 |
| 422 | 163 | 78 | 189.2 | 616 | 147 | 169 | | | >10000 |
| 423 | 4946 | 1970 | | | | | | | |
| 424 | 926 | 460 | 148.1 | | 698 | | | | >10000 |
| 425 | 60 | 166 | 57.7 | 103 | 43 | 21 | 25 | | 358 |
| 426 | 40 | 136 | 31 | | | | | | >10000 |
| 428 | 29 | 199 | | | | | | | |
| 429 | 113 | 95 | | | | | | | |
| 430 | 134 | 269 | 94.9 | 1026 | 592 | 439 | | | >10000 |
| 431 | 1413 | 1097 | 158 | | | | | | >10000 |
| 433 | 2609 | 1160 | | | | | | | |
| 435 | 172 | 578 | 40 | 215 | 164 | | | | 4200 |
| 436 | 1763 | 2946 | | | | | | | |
| 437 | 5197 | 4485 | | | | | | | |
| 438 | 5611 | 8005 | | | | | | | |
| 439 | >10000 | 6241 | | | | | | | |
| 440 | >10000 | 6510 | | | | | | | |
| 441 | >10000 | 5712 | | | | | | | |
| 442 | >10000 | >10000 | | | | | | | |
| 443 | >10000 | 8575 | | | | | | | |
| 444 | 8870 | 5683 | | | | | | | |
| 445 | >10000 | >10000 | | | | | | | |
| 447 | 202 | 179 | | 338 | 373 | 170 | 44 | | |
| 448 | 631 | 280 | 22.4 | 196 | 369 | 156 | 24 | | 1005 |
| 449 | 5758 | 2689 | 53.7 | | | | | | >10000 |
| 450 | 3492 | 1266 | | | | | | | |
| 451 | >10000 | >10000 | | | | | | | |
| 452 | 3003 | >10000 | | | | | | | |
| 453 | 9385 | 5761 | | | | | | | |
| 455 | >10000 | >10000 | | | | | | | |
| 456 | >10000 | >10000 | | | | | | | |
| 457 | >10000 | >10000 | | | | | | | |
| 458 | >10000 | >10000 | | | | | | | |
| 459 | >10000 | >10000 | | | | | | | |
| 460 | 1625 | 5364 | | | | | | | |
| 461 | >10000 | >10000 | | | | | | | |
| 462 | >10000 | >10000 | | | | | | | |
| 463 | 615 | 597 | | | | | | | |
| 464 | 1005 | 1185 | 675.8 | | | | | | >10000 |
| 465 | 68 | 154 | 16 | | | | | | 903 |
| 467 | 533 | 257 | 37.2 | 296 | 226 | | | | 841 |
| 468 | 1939 | 1767 | | | | | | | |
| 469 | 1198 | 1978 | | | | | | | |
| 470 | 362 | 50 | 1.5 | 264 | 283 | 151 | 27 | | 1843 |
| 471 | 5321 | 2884 | | | | | | | |
| 472 | 1735 | 907 | | | | | | | |
| 473 | 5120 | 4095 | | | | | | | |
| 481 | 106 | 194 | 479.6 | | 285 | | | | >10000 |
| 482 | 1455 | 1201 | | | | | | | |
| 483 | 1158 | 1222 | 1465.8 | | | | | | >10000 |
| 484 | >10000 | >10000 | | | | | | | |
| 488 | >10000 | 7507 | | | | | | | |
| 491 | >10000 | >10000 | | | | | | | |
| 492 | 9035 | 3935 | | | | | | | |
| 494 | 749 | 2576 | | | | | | | |
| 495 | 1237 | 2897 | | | | | | | |
| 496 | 10 | 19 | 16.3 | | | | | | 5354 |
| 497 | 66 | 106 | | | | | | | |
| 498 | 258 | 410 | | | | | | | |
| 499 | 41 | 168 | 186 | | | | | | >10000 |
| 500 | 25 | 203 | 37.2 | | | | | | >10000 |
| 501 | 23 | 132 | 41.2 | | | | | | >10000 |
| 502 | 124 | 1310 | | | | | | | |
| 503 | 11 | 19 | 10.7 | | | | | | 3231 |
| 504 | 3 | 8 | | 45 | 19 | 18 | 2 | | 3133 |
| 505 | 128 | 1078 | | | | | | | |
| 506 | 10 | 5 | 9.9 | 298 | 17 | 81 | 4 | | 1091 |
| 507 | 10 | 4 | 5.5 | 39 | 6 | 13 | 1 | | 1321 |
| 508 | 139 | 36 | 48.7 | | | | | | 9999 |
| 509 | 495 | 321 | | | | | | | |
| 510 | 2801 | 4339 | | | | | | | |
| 511 | 2546 | >10000 | 169.4 | | | | | | >10000 |
| 512 | 30 | 85 | 12.6 | | | | | | 276 |
| 513 | 27 | 92 | 6 | | | | | | 949 |
| 574 | 13 | 25 | 0.4 | 18 | 16 | 12 | 5 | | 442 |
| 575 | 49 | 128 | | 97 | 51 | 41 | 19 | | 889 |
| 576 | 88 | 57 | 5.8 | 137 | 61 | 57 | 49 | | 2763 |
| 577 | 2 | 6 | 0.1 | 2 | 1 | 1 | 1 | | 7 |
| 578 | 1 | 2 | 0.3 | 36 | 4 | 7 | 1 | | 1042 |
| 579 | 699 | 513 | 18.3 | 968 | 168 | 227 | 204 | | >10000 |
| 580 | 16 | 71 | 1 | 71 | 26 | 17 | 14 | | 405 |
| 581 | 1137 | 689 | | | | | | | >10000 |
| 582 | 79 | 204 | 3.1 | 219 | 86 | 63 | 40 | | 2485 |
| 583 | 247 | 177 | | 98 | 43 | 50 | 20 | | 2115 |
| 585 | 273 | 191 | 19.7 | 439 | 125 | 163 | 111 | | 6505 |
| 586 | 19 | 29 | 38 | 336 | 48 | 137 | 23 | | 8156 |
| 587 | 131 | 164 | 3.3 | 82 | 33 | 25 | 11 | | 170 |
| 588 | 460 | 779 | 250.1 | 544 | 455 | 276 | 211 | | 6389 |
| 589 | 6 | 3 | 2.5 | 30 | 6 | 16 | 2 | | 1081 |
| 590 | 391 | 277 | 40.8 | | | | | | 9596 |
| 592 | 14 | 8 | 11 | 148 | 29 | 50 | 4 | | 3447 |
| 593 | 19 | 50 | 49.1 | 515 | 114 | 158 | 16 | | 9362 |
| 594 | 2491 | 1122 | | | | | | | |
| 595 | 1002 | 913 | | | | | | | |
| 596 | 1919 | 2489 | >10000 | 3340 | 2668 | 1278 | 595 | | >10000 |
| 597 | 4471 | 3254 | | | | | | | |
| 598 | 2385 | 1743 | 1452.2 | 4278 | 1537 | 1255 | 757 | | >10000 |
| 599 | 3698 | 3252 | | | | | | | |
| 600 | 4985 | 6288 | 1123.3 | 3292 | >10000 | 1211 | 808 | | 605 |
| 601 | 6796 | 4225 | | | | | | | |
| 602 | >10000 | >10000 | | >10000 | >10000 | 7580 | 907 | | |
| 603 | >10000 | 3832 | | | | | | | |
| 604 | >10000 | >10000 | | | | | | | |
| 605 | 3364 | 1910 | | | | | | | |
| 606 | >10000 | >10000 | | | | | | | |
| 607 | 161 | 1215 | 953.1 | 3575 | 722 | 3407 | 229 | | >10000 |
| 608 | 46 | 274 | 49.7 | 544 | 215 | 353 | 42 | | >10000 |
| 609 | 8 | 32 | 47 | 65 | 33 | 30 | 5 | | 8638 |
| 610 | 4 | 19 | 29.1 | 312 | 51 | 111 | 15 | | >10000 |
| 611 | 3 | 5 | 8.7 | 145 | 7 | 39 | 3 | | 4021 |
| 612 | 58 | 41 | 19.4 | 92 | 83 | 129 | 16 | | 1136 |
| 613 | 2 | 3 | 2.9 | 36 | 4 | 12 | 1 | | 1828 |
| 614 | 4 | 10 | 5.5 | 123 | 3 | 40 | 4 | | 4073 |
| 615 | 7 | 4 | 34.6 | 144 | 15 | 41 | 6 | | 4641 |
| 616 | 17 | 17 | 62.9 | 176 | 24 | 48 | 11 | | >10000 |
| 617 | 13 | 15 | | 115 | 27 | 40 | 8 | | |
| 618 | 5 | 4 | | 59 | 15 | 26 | 3 | | 1393 |
| 619 | 5 | 7 | 1.9 | 69 | 10 | 34 | 1 | | 1425 |
| 620 | 6 | 7 | 3.7 | 111 | 24 | 30 | 2 | | 2669 |
| 621 | 5 | 11 | | 52 | 12 | 22 | 4 | | 2486 |
| 622 | 2 | 4 | | 40 | 6 | 14 | 1 | | 1057 |
| 623 | 1 | 2 | 1.6 | 31 | 5 | 12 | 1 | | 1424 |
| 624 | 4 | 6 | 10.5 | 66 | 19 | 22 | 4 | | 2467 |
| 627 | >10000 | >10000 | | | | | | | |
| 628 | 2 | 13 | 1.6 | 78 | 7 | 32 | 3 | | >10000 |
| 629 | 2553 | 1666 | | | | | | | |
| 630 | 361 | 753 | | | | | | | |
| 631 | 69 | 356 | 48.3 | 508 | 68 | 341 | 24 | | 7143 |
| 632 | 12 | 32 | 4.1 | 55 | 19 | 21 | 6 | | 716 |
| 633 | 48 | 100 | 70.3 | | | | | | >10000 |
| 634 | 102 | 284 | 103.1 | 1415 | 346 | 585 | 45 | | >10000 |
| 635 | 134 | 230 | 157.8 | 1318 | 609 | 567 | 87 | | >10000 |
| 636 | 29 | 153 | 81.1 | 1106 | 200 | 418 | 45 | | >10000 |
| 637 | 11 | 9 | 1.4 | | | | | | 5995 |
| 641 | 179 | 216 | 302.8 | 1011 | 225 | 481 | 109 | | >10000 |
| 642 | 59 | 200 | 119.8 | 738 | 145 | 325 | 105 | | >10000 |
| 643 | 27 | 211 | 39.9 | 312 | 266 | 67 | 30 | | 4882 |
| 645 | 4640 | 4639 | | | | | | | |
| 646 | 6300 | 4636 | | | | | | | |
| 647 | >10000 | >10000 | | | | | | | |
| 648 | 1941 | 2328 | 101.4 | 570 | 1128 | 184 | 710 | | >10000 |
| 649 | 6525 | 3708 | | >10000 | 3097 | 2436 | 1105 | | |
| 650 | >10000 | >10000 | | | | | | | |
| 651 | 572 | 84 | 23 | 516 | 94 | 165 | 45 | | >10000 |
| 652 | >10000 | >10000 | | | | | | | |
| 653 | 70 | 60 | 2.4 | 28 | 17 | 12 | 6 | | 131 |
| 654 | 197 | 323 | 38.7 | 191 | 334 | 131 | 72 | | 2661 |
| 655 | 247 | 525 | | 926 | 359 | 237 | 189 | | |
| 656 | 82 | 100 | 4.5 | 1219 | 294 | 307 | 54 | | >10000 |
| 657 | 46 | 3037 | 94.9 | | 180 | | 122 | | |
| 658 | 7 | 11 | 0.6 | | 7 | | 2 | | |
| 659 | 38 | 79 | 1.2 | | 24 | | 8 | | |
| 660 | 261 | 204 | 28.2 | | 131 | | 25 | | |
| 661 | >10000 | 4778 | 1379.2 | | 3804 | | 864 | | |
| 709 | 4294 | 4722 | | | 8710 | | 1253 | | |
| 710 | 626 | 1107 | 75.9 | 1860 | 1207 | 813 | 484 | 221 | >10000 |
| 711 | 1936 | 7409 | 830.5 | 7221 | 4819 | 2519 | 2677 | 424 | >10000 |
| 712 | 70 | 421 | 1.4 | 240 | 216 | 141 | 30 | 19 | 806 |
| 713 | 51 | 67 | 10.5 | 253 | 92 | 127 | 29 | 17 | 1803 |
| 715 | 2 | 4 | | | 1 | | 1 | | |

TABLE 4
Inhibition (HTRF % at 100 nM) of KRas -mediated Phosphorylation
of ERK by Exemplary Compounds of Formula (I)

| Ex. No. | AsPC-1 | H727 | H460 | H358 | A549 | HCT116 |
|---|---|---|---|---|---|---|
| 233 | 30 | 31 | −7 | | | −10 |
| 234 | 34 | 22 | −4 | −28 | 9 | 14 |
| 235 | 30 | −5 | 2 | 8 | 13 | 5 |
| 237 | 10 | 22 | −16 | 7 | 30 | 5 |
| 238 | 9 | −2 | | −17 | −3 | |
| 239 | 6 | −8 | 11 | −8 | −9 | −5 |
| 240 | −5 | −18 | | −1 | −1 | |
| 241 | 5 | −4 | | −15 | 5 | |
| 242 | −9 | 2 | | 3 | 6 | |
| 244 | 70 | 42 | 54 | 86 | 59 | 8 |
| 245 | 12 | 15 | 12 | 5 | 4 | 10 |
| 246 | 63 | 32 | 37 | 62 | 41 | 0 |
| 247 | | | 61 | 99 | 73 | 35 |
| 249 | 1 | 21 | 53 | 70 | 35 | 10 |
| 250 | −2 | 25 | 36 | 34 | 36 | 26 |
| 251 | −4 | 3 | 15 | 2 | 21 | −7 |
| 252 | −5 | 0 | 3 | −7 | 16 | 14 |
| 253 | 5 | −12 | 7 | 7 | −12 | 2 |
| 254 | −16 | −36 | 6 | 24 | 11 | −22 |
| 255 | −7 | −18 | 5 | 17 | −2 | 14 |
| 256 | | | 7 | 25 | 34 | −4 |
| 257 | 77 | 36 | 21 | 95 | 35 | 8 |
| 258 | 79 | 55 | 24 | 95 | 42 | 3 |
| 259 | 79 | 29 | 14 | 66 | 6 | −11 |
| 260 | 98 | 54 | 38 | 97 | 73 | 29 |
| 261 | 89 | 29 | 37 | 88 | 40 | 15 |
| 262 | 94 | 38 | 21 | 91 | 47 | 16 |
| 263 | | | −31 | | | −11 |
| 264 | 34 | 22 | 0 | 43 | 15 | −7 |
| 265 | 63 | 40 | −4 | 60 | 32 | 16 |
| 268 | 84 | 49 | | 104 | 67 | |
| 269 | | | 89 | 100 | 93 | |
| 270 | | | 34 | 94 | 52 | 36 |
| 271 | | | 34 | 94 | 31 | 19 |
| 272 | | | 22 | −5 | −15 | 19 |
| 273 | | | 5 | 6 | 22 | 10 |
| 274 | | | −1 | −5 | 5 | 2 |
| 275 | | | 16 | 78 | 59 | −10 |
| 276 | | | −17 | 42 | 16 | −1 |
| 277 | | | 12 | 64 | 25 | −2 |
| 278 | | | −10 | 12 | 22 | −6 |
| 279 | | | 0 | | | 4 |
| 280 | | | | 14 | −20 | |
| 282 | | | 35 | 88 | 37 | 12 |
| 283 | | | 18 | 48 | 40 | −8 |
| 284 | | | 53 | 89 | 66 | 27 |
| 286 | 96 | 80 | 66 | 97 | 87 | 46 |
| 287 | | | | −20 | −15 | |
| 288 | | | 3 | 26 | 22 | 17 |
| 289 | | | 5 | −2 | −12 | 13 |
| 290 | | | −28 | 15 | 1 | 8 |
| 291 | | | 4 | 20 | 23 | 12 |
| 292 | | | 93 | 100 | 98 | 75 |
| 293 | | | 66 | 95 | 86 | 40 |
| 294 | | | 90 | 100 | 82 | 72 |
| 295 | | | 84 | 97 | 60 | 63 |
| 296 | 94 | 67 | 78 | 98 | 86 | 51 |
| 297 | | | 12 | 8 | 19 | 5 |
| 299 | | | −5 | −14 | −11 | −4 |
| 300 | | | 59 | 42 | 38 | 36 |
| 301 | | | 54 | 102 | 85 | 34 |
| 302 | 91 | 70 | 66 | 92 | 66 | 33 |
| 303 | | | 34 | 98 | 62 | 3 |
| 304 | | | 25 | 43 | 33 | −15 |
| 305 | | | 6 | 9 | 4 | 18 |
| 306 | 99 | 80 | 81 | 100 | 77 | 51 |
| 307 | | | 82 | 102 | 76 | 53 |
| 308 | | | 41 | 93 | 62 | 23 |
| 309 | | | 6 | 12 | 7 | −8 |
| 310 | | | 26 | 96 | 42 | 19 |
| 311 | | | 86 | 96 | 76 | 74 |
| 312 | | | 82 | 94 | 88 | 34 |
| 313 | | | 31 | 8 | 7 | 2 |
| 314 | | | 13 | 0 | 3 | 4 |
| 315 | | | 4 | 11 | −26 | 2 |
| 316 | | | −20 | −22 | −4 | −2 |
| 317 | | | 1 | 19 | 12 | 13 |
| 318 | | | 91 | | | |
| 319 | | | −10 | | | 8 |
| 320 | 99 | 70 | 56 | 103 | 90 | 17 |
| 321 | | | 16 | 7 | 14 | 5 |
| 322 | 98 | 80 | 88 | 104 | 86 | 77 |
| 323 | 102 | 77 | 81 | 102 | 96 | 72 |
| 324 | | | 21 | 4 | 11 | −1 |
| 325 | | | 76 | 99 | 88 | 52 |
| 326 | | | 48 | 13 | 34 | 26 |
| 327 | | | 66 | 97 | 88 | 47 |
| 328 | | | 54 | 94 | 72 | 8 |
| 329 | 95 | 76 | 76 | 98 | 70 | 47 |
| 330 | 94 | 77 | 60 | 98 | 80 | 27 |
| 331 | | | 88 | 97 | 87 | 74 |
| 332 | | | 80 | 100 | 87 | 38 |
| 333 | 102 | 89 | 98 | 100 | 74 | 88 |
| 334 | | | −1 | 2 | 12 | 12 |
| 335 | | | 10 | 0 | 13 | 15 |
| 336 | | | 1 | −26 | −24 | −11 |
| 337 | 99 | 80 | 96 | 106 | 94 | 73 |
| 338 | 95 | 76 | 73 | 102 | 78 | 38 |
| 339 | 97 | 74 | 59 | 95 | 80 | 20 |
| 340 | 96 | 80 | 69 | 96 | 82 | 32 |
| 341 | 95 | 76 | 77 | 91 | 75 | 47 |
| 342 | 96 | 80 | 86 | 100 | 87 | 59 |
| 343 | | | 7 | −13 | 4 | −2 |
| 344 | | | 88 | 97 | 85 | 57 |
| 345 | | | −9 | 13 | 12 | 5 |
| 346 | | | 9 | 11 | 19 | 7 |
| 347 | | | 21 | 5 | 28 | −14 |
| 349 | 96 | 86 | 84 | 101 | 75 | 47 |
| 350 | | | 44 | 76 | 23 | 10 |
| 351 | | | 1 | −7 | 11 | 5 |
| 352 | | | −4 | −7 | −15 | −2 |
| 353 | | | −15 | −10 | 26 | 6 |
| 354 | | | 8 | | | 11 |
| 355 | | | 18 | | | −28 |
| 356 | | | 28 | | | −2 |
| 357 | | | 12 | | | −20 |
| 358 | 95 | 72 | 90 | | | 50 |
| 359 | | | 22 | | | 1 |
| 360 | | | 87 | | | 49 |
| 361 | | | 1 | | | −11 |
| 362 | | | 60 | | | 27 |
| 363 | | | 52 | | | 26 |
| 364 | | | 13 | | | 10 |
| 365 | | | 56 | | | 32 |
| 366 | | | 59 | | | 37 |
| 367 | | | 83 | 101 | 85 | 62 |
| 368 | | | 31 | | | 1 |
| 369 | | | 36 | 80 | 11 | 13 |
| 370 | | | | 97 | 65 | |
| 371 | | | | 101 | 79 | |
| 372 | 95 | 76 | | 104 | 72 | |
| 373 | | | | 93 | 72 | |
| 374 | | | | 99 | 57 | |
| 375 | | | | 102 | 68 | |
| 376 | | | | 70 | 34 | |
| 378 | | | | 105 | 69 | |
| 379 | | | | 105 | 91 | |
| 380 | 94 | 69 | | 93 | 81 | |
| 381 | | | | 98 | 85 | |
| 382 | | | | 99 | 80 | |
| 383 | 97 | 85 | | 98 | 90 | |
| 384 | 98 | 79 | | 100 | 65 | |
| 385 | | | | 94 | 74 | |
| 386 | | | | 91 | 88 | |
| 387 | 99 | 85 | | 100 | 89 | |
| 388 | | | | 97 | 79 | |
| 389 | | | | 23 | −36 | |
| 390 | | | | 98 | 79 | |
| 391 | | | | 77 | 69 | |
| 392 | | | | 47 | 38 | |
| 393 | | | | 83 | 41 | |
| 394 | | | | 80 | 57 | |
| 395 | | | | 52 | 14 | |
| 425 | 15 | −10 | 74 | 93 | 65 | 39 |
| 426 | 26 | 17 | 1 | 55 | 35 | 5 |
| 427 | −23 | −14 | 25 | −16 | 0 | 4 |
| 428 | | | 68 | | | 30 |
| 429 | | | −3 | 19 | 23 | −4 |
| 433 | −5 | 0 | 6 | −7 | 9 | 1 |
| 436 | −12 | 12 | −4 | 10 | 3 | 5 |
| 437 | 2 | −2 | | −16 | −10 | |
| 438 | −4 | −7 | | | | |
| 440 | −10 | −10 | | −1 | −6 | |
| 441 | 9 | −17 | | 11 | −33 | |
| 442 | 9 | −4 | | | | |
| 443 | −16 | −25 | | | | |
| 445 | −9 | 13 | | | | |
| 446 | 21 | 13 | 27 | | | 0 |
| 447 | 23 | 24 | 35 | 61 | 28 | 28 |
| 462 | −6 | −8 | 9 | 1 | 8 | 3 |
| 463 | 15 | 20 | 2 | −13 | 18 | −3 |
| 464 | | | −24 | | | 14 |
| 465 | | | 54 | 66 | 46 | 25 |
| 468 | | | −7 | −2 | −12 | −4 |
| 469 | | | 2 | 0 | −1 | 0 |
| 470 | | | 39 | 77 | 33 | −5 |
| 471 | | | −3 | | | −3 |
| 472 | | | 19 | 25 | −2 | 8 |
| 473 | | | | 12 | 12 | |
| 481 | | | −1 | | | 8 |
| 485 | 0 | 8 | | | | |
| 487 | 16 | 0 | | | | |
| 488 | 13 | 5 | 14 | | | −1 |
| 490 | 9 | −12 | | | | |
| 492 | −11 | 0 | 6 | −3 | −24 | 23 |
| 493 | 11 | −4 | | | | |
| 494 | −4 | 13 | 4 | 8 | 14 | 4 |
| 495 | −1 | 3 | | 1 | 26 | |
| 496 | 96 | 71 | | 97 | 95 | |
| 498 | −13 | 12 | | 5 | 25 | |
| 499 | 47 | 37 | 6 | 57 | 25 | 11 |
| 500 | 91 | 20 | 22 | 74 | 46 | 11 |
| 501 | 86 | 40 | 17 | 95 | 27 | 23 |
| 502 | 22 | 7 | 7 | 5 | 11 | 8 |
| 504 | 100 | 84 | 89 | | | 61 |
| 505 | 30 | 26 | 0 | | | 7 |
| 506 | 97 | 80 | 71 | | | 34 |
| 507 | 96 | 79 | 84 | 101 | 76 | 51 |
| 508 | 8 | 37 | 47 | 72 | 33 | 7 |
| 510 | 16 | 19 | 9 | −5 | −1 | 0 |
| 511 | 18 | 9 | −7 | −22 | 1 | 4 |
| 512 | 74 | 42 | 79 | 92 | 71 | 56 |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A compound of Formula (I): 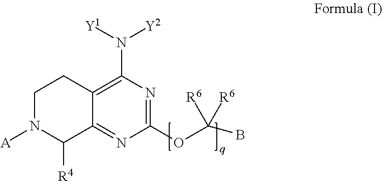
or a pharmaceutically acceptable salt thereof, wherein:
A is aryl or heteroaryl, wherein the aryl or the heteroaryl is optionally substituted with 1-4 R¹;
B is selected from: 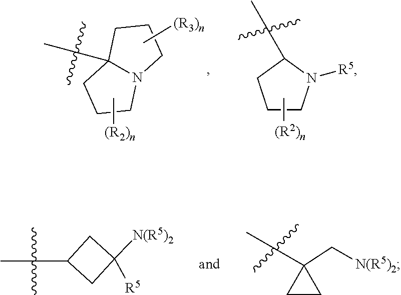
Y¹ is L-hydrogen optionally substituted with 1-4 R⁸, hydroxy, halogen, L-C3-C6 cycloalkyl optionally substituted with 1-4 R⁹, L-S(0)₂N(R⁵)₂ optionally substituted with 1-4 R⁹, L-heteroaryl optionally substituted with 1-4 R⁸, L-aryl optionally substituted with 1-4 R⁸, and L-heterocycle substituted with 1-2 oxo (═O) or oxo-containing substituent and optionally further substituted with 1-2 heteroaryl-R^{g} or R⁸
Y² is hydrogen or C1-C4 alkyl;
or Y¹ and Y² join to form: 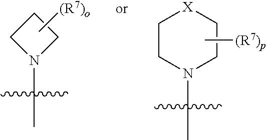
where X is selected from: a bond, —S—, —O—, —N<bound to a fused ring, —CH₂—, —CH₂—NH—, —CH₂—NH—CH₂—, —CH₂—CH₂—CH₂—, —CH₂—CH₂—, —O—CH₂— and —S—CH₂—;
each R¹ is independently halogen, cyano, hydroxy, C1-C4 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, —S—C1-C3 haloalkyl, C1-C3 alkoxy, hydroxyC1-C3 alkyl, —CH₂C(═O)N(RS)₂, —C3-C4 alkynyl(NRS)₂, —N(RS)₂, deuteroC2-C4 alkynyl, (C1-C3 alkoxy)haloC1-C3 alkyl-, or C3-C6 cycloalkyl wherein said C3-C6 cycloalkyl is optionally substituted with halogen or C1-C3 alkyl;
each R² is independently hydrogen, hydroxy, halogen, C1-C3 alkyl, C1-C3 cyanoalkyl, C1-C3 hydroxyalkyl, HC(═O)—, —OC(O)N(R⁵)₂, —CH₂OC(O)N(RS)₂, —CH₂NR⁵—SO₂—N(R⁵)₂, —CO₂R⁵, —CO₂N(R⁵)₂, ═CH₂, ═CHR″ or ═C(R″)₂ each R³ is independently hydrogen, hydroxy, halogen, L-C1-C3 alkyl, C1-C3 cyanoalkyl, C1-C3 hydroxyalkyl, HC(═O)—, —OC(O)N(R⁵)₂, —CH₂OC(O)N(RS)₂, —CH₂NR⁵—SO₂—N(RS)₂, —CO₂R⁵, —CO₂N(R⁵)₂; ═CH₂, ═CHR″ or ═C(R″)₂;
wherein at least one of R² and R³ are ═CH₂, ═CHR″ ¹ or ═C(R″)₂;
R⁴ is hydrogen, halogen or C1 -C3 alkyl;
each R⁵ is independently hydrogen, cyclopropyl or C1-C3 alkyl;
each R⁶ is independently hydrogen, hydroxy, C1-C4 hydroxyalkyl or heteroaryl, or two R⁶ join to form C₃-C₆ cycloalkyl or heterocycle;
each R⁷ is independently hydrogen, C1-C3 alkyl, hydroxy, halogen, halo-C1-C3 alkyl, di-halo-C1-C3 alkyl, tri-halo-C1-C3 alkyl, —NH₂, —NH(C1-C3 alkyl), —N(C1-C3 alkyl)₂, oxo (═O), —O—(C1-C3 alkyl), —(C1-C3 alkyl)—OH, —C(O)OH, —C(O)O(C1-C3 alkyl), —O—CH₂-C(O)NH₂, L—C(O)NH₂, —C(O)NH(C1-C3 alkyl), —NHC(O)(C1-C3 alkyl), —C(O)N(C1-C3 alkyl)₂, —CN, aryl, dialkylphosphine oxide, —S(O)₂NH(CH₃), sulfone, L-heterocycle optionally substituted with 1-2 substituents selected from L-hydroxy, oxo (═O), C1-C3 alkyl and C3 cycloalkyl, or L-heteroaryl optionally substituted with 1-2 substituents selected from L-hydroxy, —NH₂, C1-C3 alkyl, C1-C3 haloalkyl, C3 cycloalkyl, —C(O)NH(C3-C4 cycloalkyl) and —NHC(O)(C1-C3 alkyl), two R⁷ on the same atom optionally join to form a spirocyclic ring selected from C3-C6 cycloalkyl and heterocycle, where said spirocyclic ring is optionally substituted with 1-2 substituents selected from oxo (═O), halogen, hydroxy, C1-C3 alkyl and —O-(C1-C3 alkyl), two R⁷ on adjacent atoms optionally join to form a bond or a fused ring selected from C3-C6 cycloalkyl optionally substituted with 1-4 R⁸, heteroaryl optionally substituted with 1-4 R⁸, aryl optionally substituted with 1-4 R⁸, and heterocycle optionally substituted with 1-4 R⁸, and two R⁷ on non-adjacent atoms optionally join to form a 1-2 carbon bridge;
each R⁹ is independently C1-C3 alkyl, L-hydroxy, halogen, halo-C1-C3 alkyl, di-halo-C1-C3 alkyl, tri-halo-C1-C3 alkyl, —N(R⁵)₂, oxo (═O), —O-(C1-C3 alkyl), —(C1-C3 alkyl)—OH, —C(O)OR⁵, —C(O)B, —C(OR⁵)(R⁵)₂, —(C1-C3 alkyl)C(O)N(RS)₂, —C(O)N(R⁵)₂, —C(O)N(R¹⁰)₂, —CN, L-L-heteroaryl or L-heterocycle each optionally substituted with C1-C3 alkyl, C1-C3 haloalkyl, —CH₂—S—CH₃, —S(O)₂NH₂ or —S(O)₂(C1-C3 alkyl);
each R⁹ is independently C1-C3 alkyl, hydroxy, halogen, oxo (═O), —O-(C1-C3 alkyl), —(C1-C3 alkyl)—OH, —C(O)OH, —C(O)O(C1-C3 alkyl), —C(O)NH₂, —C(O)NH(C1-C3 alkyl), —C(O)N(C1-C3 alkyl)₂, L-heteroaryl or —CN, or two R⁹ join to form a bond or —S(O)(CH₃)₂;
each R¹⁰ is independently hydrogen, C1-C3 alkyl, halogen, or joins with R⁷ or another R¹⁰ to form a heterocyclic ring;
each R″ is independently halogen;
each L is independently a bond; —C1-C4 alkylene-, —NR⁵—, or —C(O)—;
each n is 0-3;
o is 1-6;
p is 1-8; and
q is 0-1.

2. The compound or salt of claim 1, wherein
q is 1;
A is naphthyl; and
B is:

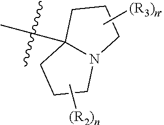

3. The compound or salt of claim 1, wherein:
q is 1;
A is naphthyl; and
B is:

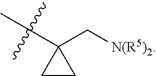

4. The compound or salt of any of claims 1-3, wherein Y¹ is hydrogen, hydroxy, halogen or L-heteroaryl optionally substituted with 1-4 R⁸, and Y² is hydrogen or C1-C4 alkyl.

5. The compound or salt of any of claims 1-3, wherein Y¹ and Y² join to form:

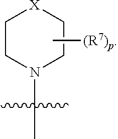

6. The compound of claim 5, wherein X is —CH₂—NH—, and two R⁷ join to form a fused heteroaryl ring substituted with 1-4 R⁸ where one R⁸ is —C(O)N(R¹⁰)₂.

7. The compound of claim 6, wherein the fused heteroaryl ring is pyrazolyl, one R⁸ is —C(O)N(R¹⁰)₂ and one R⁸ is halogen or C1-C3 alkyl.

8. The compound of claim 5, wherein X is a bond, and two R⁷ join to form a fused heterocyclyl ring, optionally substituted with one or two oxo.

9. The compound of claim 5, wherein X is —CH₂—, and two R⁷ join to form a spirocyclic heterocyclyl ring substituted with one or two oxo.

10. The compound or salt of any of claims 1-3, wherein at least one R¹ is C1-C4 alkyl.

11. The compound or salt of any of claims 1-3, wherein at least one R¹ is halogen.

12. The compound or salt of claim 11, wherein said halogen is a fluorine.

13. The compound or salt of any of claims 1-3, wherein at least one R¹ is hydroxy.

14. The compound or salt of any of claims 1-3, wherein one R² is C1-C4 alkyl.

15. The compound or salt of any of claims 1-3, wherein at least one R² is halogen.

16. The compound or salt of claim 15, wherein said halogen is a fluorine.

17. The compound or salt of any of claims 1-3, wherein at least one R² is hydroxy.

18. The compound or salt of any of claims 1-3, wherein at least one R³ is C1-C4 alkyl.

19. The compound or salt of any of claims 1-3, wherein at least one R³ is halogen.

20. The compound or salt of claim 19, wherein said halogen is fluorine.

21. The compound or salt of any of claims 1-3, wherein at least one R² is ═CH₂, ═CHR″ or ═C(R¹)₂.

22. The compound or salt of claim 21, wherein R″ is F.

23. The compound or salt of any of claims 1-3, wherein at least one R³ is hydroxy.

24. The compound or salt of any of claims 1-3, wherein at least one R³ is ═CH₂, ═CHR″ or ═C(R″)₂.

25. The compound or salt of claim 24, wherein R¹¹ is F.

26. The compound or salt of any of claims 1-3, wherein R⁴ is halogen.

27. The compound or salt of claim 26, wherein said halogen is fluorine.

28. The compound or salt of any of claims 1-3, wherein at least one R⁵ is C1-C4 alkyl.

29. The compound or salt of any of claims 1-3, wherein at least one R⁵ is hydrogen.

30. The compound or salt of any of claims 1-3, wherein one or both R⁶ are hydrogen or C1-C4 alkyl.

31. The compound or salt of any of claims 1-3, wherein two R⁶ join to form C3-C6 cycloalkyl or heterocycle.

32. The compound or salt of any of claims 1-3, wherein Y¹ is L-C₃-C₆ cycloalkyl, L-heteroaryl, L-aryl, or L-heterocycle, where L is a bond, C1-C4 alkyl, NH or N(C1-C3) alkyl.

33. The compound or salt of claim 32, wherein Y¹ is L-heteroaryl.

34. The compound or salt of claim 33, wherein the heteroaryl is thietane dioxide, iso-thiazolidine dioxide, imidazopyrazine, pyridine or pyrimidine.

35. The compound or salt of claim 32, wherein Y¹ is L-C3-C6 cycloalkyl.

36. The compound or salt of claim 35, wherein the cycloalkyl is cyclobutane, cyclopentane, cyclohexane or cycloheptane.

37. The compound or salt of claim 32, wherein Y¹ is L-heterocycle.

38. The compound or salt of claim 37, wherein the heterocycle is pyrrolidinone.

39. The compound or salt of any of claims 1-3, wherein Y² is hydrogen.

40. The compound or salt of any of claims 1-3, wherein Y² is C1-C4 alkyl;
41. The compound or salt of any of claims 1-3, wherein at least one R⁸ is C1-C4 alkyl.

42. The compound or salt of any of claims 1-3, wherein at least one R⁸is hydroxy or C1-C3 alkyl-hydroxy.

43. The compound or salt of any of claims 1-3, wherein one or two R⁸ are oxo (═O).

44. The compound or salt of any of claims 1-3, wherein at least one R is aryl or heteroaryl.

45. The compound or salt of any of claims 1-3, wherein at least one R⁸ is C(O)OH.

46. The compound or salt of any of claims 1-3, wherein at least one R⁸ is —C(O)NH₂, —C(O)NH(C1-C3 alkyl) or —C(O)N(C1-C3 alkyl)₂.

47. The compound or salt of any of claims 1-3, wherein at least one R⁸ is —NH₂, —NH(C1-C3 alkyl);
—N(C1-C3 alkyl)₂.

48. The compound or salt of any of claims 1-3, wherein at least one R⁹ is C1-C4 alkyl.

49. The compound or salt of any of claims 1-3, wherein at least one R⁹ is hydroxy or C1-C3 alkyl-hydroxy.

50. The compound or salt of any of claims 1-3, wherein one or two R⁹ is oxo (═O).

51. The compound or salt of any of claims 1-3, wherein at least one R⁹ is aryl or heteroaryl.

52. The compound or salt of any of claims 1-3, wherein at least one R⁹ is C(O)OH.

53. The compound or salt of any of claims 1-3, wherein at least one R⁹ is —C(O)NH₂, —C(O)NH(C1-C3 alkyl) or —C(O)N(C1-C3 alkyl)2.

54. The compound or salt of any of claims 1-3, wherein Y¹ and Y² join to form piperidine, azepane, azocane, thiazepine, diazepane, oxazepane, azetidine, pyrrolidine, piperazine bound to a fused ring via nitrogen or thiomorpholine.

55. The compound or salt of any of claims 1-3, wherein two R⁷ on the same atom join to form a spirocyclic ring selected from C₃-C₆ cycloalkyl and heterocycle, where said spirocyclic ring is optionally substituted with one or more substituents selected from oxo (═O), halogen, hydroxy, C1-C3 alkyl and —O-(C1-C3 alkyl).

56. The compound or salt of any of claims 1-3, wherein two R⁷ on adjacent atoms join to form a bond or a fused ring selected from C3-C6 cycloalkyl optionally substituted with 1-4 R⁸; heteroaryl optionally substituted with 1-4 R⁸; aryl optionally substituted with 1-4 R⁸, and heterocycle optionally substituted with 1-4 R¹.

57. The compound or salt of any of claims 1-3, wherein two R⁷ on non-adjacent atoms join to form a 1-2 carbon bridge.

58. The compound or salt of any of claims 1-3, wherein one R¹⁰ is hydrogen, C1-C3 alkyl or halogen, and another R¹⁰ joins with R⁷ to form a heterocyclic ring.

59. The compound or salt of any of claims 1-3, wherein two R¹⁰ join to form a heterocyclic ring.

60. The compound or salt of any of claims 1-3, wherein each R¹⁰ is independently hydrogen, C1-C3 alkyl or halogen.

61. A compound selected from 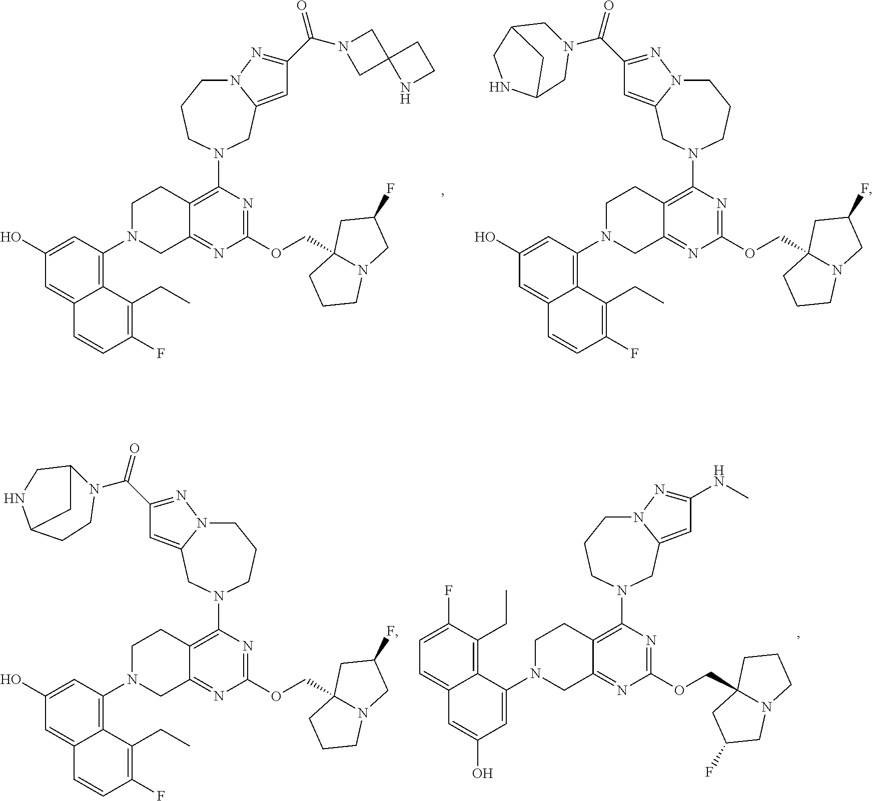 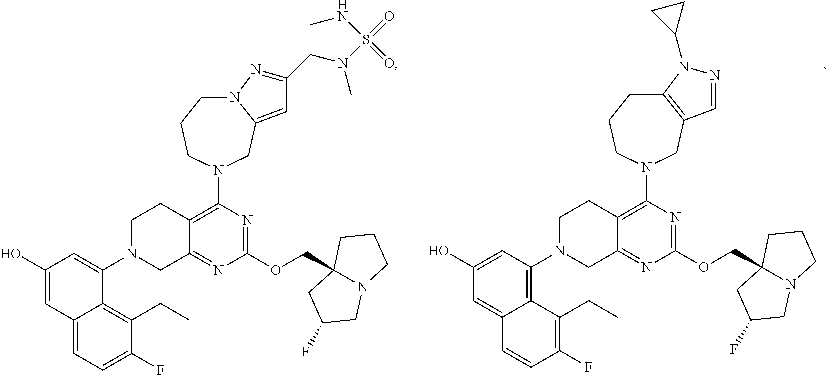 [SMILES: CCc1c(F)ccc2cc(O)cc(N3CCc4c(nc(OC[C@@]56CCCN5C[C@H](F)C6)nc4N4CCCn5nc(CN(C)S(=O)(=O)NC)cc5C4)C3)c12 ; CCc1c(F)ccc2cc(O)cc(N3CCc4c(nc(OC[C@@]56CCCN5C[C@H](F)C6)nc4N4CCCc5c(cnn5C5CC5)C4)C3)c12] 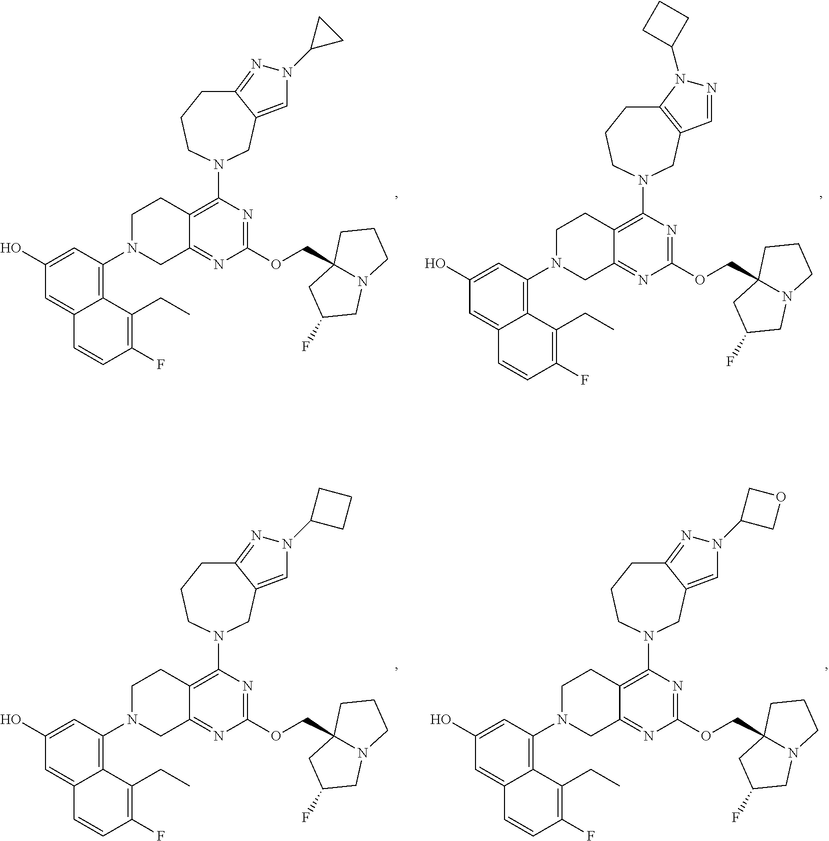 [SMILES: CCc1c(F)ccc2cc(O)cc(N3CCc4c(nc(OC[C@@]56CCCN5C[C@H](F)C6)nc4N4CCCc5nn(C6CC6)cc5C4)C3)c12 ; CCc1c(F)ccc2cc(O)cc(N3CCc4c(nc(OC[C@@]56CCCN5C[C@H](F)C6)nc4N4CCCc5c(cnn5C5CCC5)C4)C3)c12 ; CCc1c(F)ccc2cc(O)cc(N3CCc4c(nc(OC[C@@]56CCCN5C[C@H](F)C6)nc4N4CCCc5nn(C6CCC6)cc5C4)C3)c12 ; CCc1c(F)ccc2cc(O)cc(N3CCc4c(nc(OC[C@@]56CCCN5C[C@H](F)C6)nc4N4CCCc5nn(C6COC6)cc5C4)C3)c12] 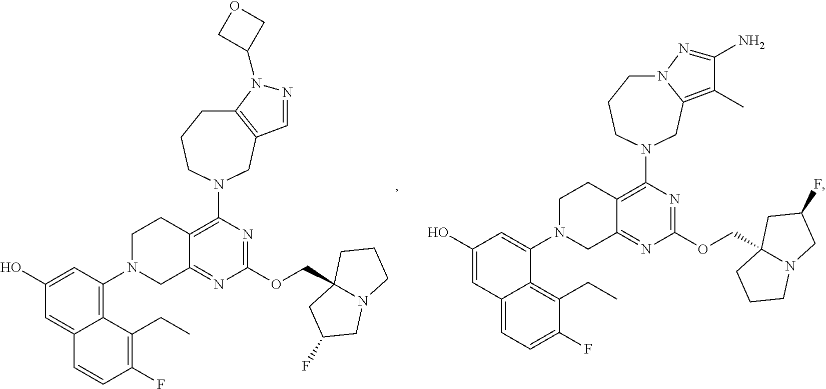 [SMILES: CCc1c(F)ccc2cc(O)cc(N3CCc4c(nc(OC[C@@]56CCCN5C[C@H](F)C6)nc4N4CCCc5c(cnn5C5COC5)C4)C3)c12 ; CCc1c(F)ccc2cc(O)cc(N3CCc4c(nc(OC[C@@]56CCCN5C[C@H](F)C6)nc4N4CCCn5nc(N)c(C)c5C4)C3)c12] 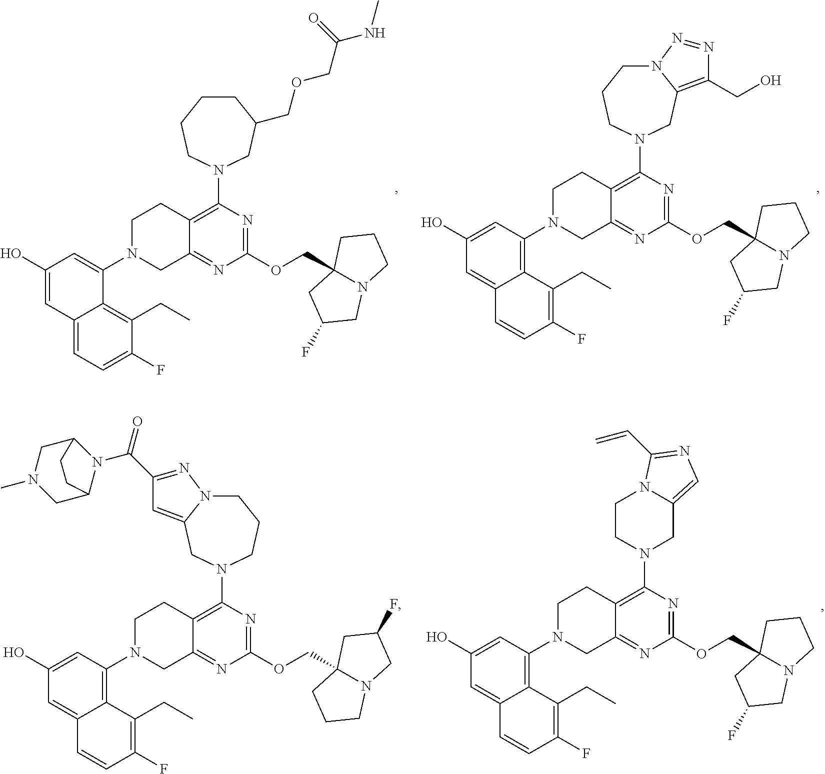 [SMILES: CCc1c(F)ccc2cc(O)cc(N3CCc4c(nc(OC[C@@]56CCCN5C[C@H](F)C6)nc4N4CCCCC(COCC(=O)NC)C4)C3)c12 ; CCc1c(F)ccc2cc(O)cc(N3CCc4c(nc(OC[C@@]56CCCN5C[C@H](F)C6)nc4N4CCCn5nnc(CO)c5C4)C3)c12 ; CCc1c(F)ccc2cc(O)cc(N3CCc4c(nc(OC[C@@]56CCCN5C[C@H](F)C6)nc4N4CCCn5nc(C(=O)N6C7CCC6CN(C)C7)cc5C4)C3)c12 ; C=Cc1ncc2n1CCN(c1nc(OC[C@@]34CCCN3C[C@H](F)C4)nc3c1CCN(c1cc(O)cc4ccc(F)c(CC)c14)C3)C2] 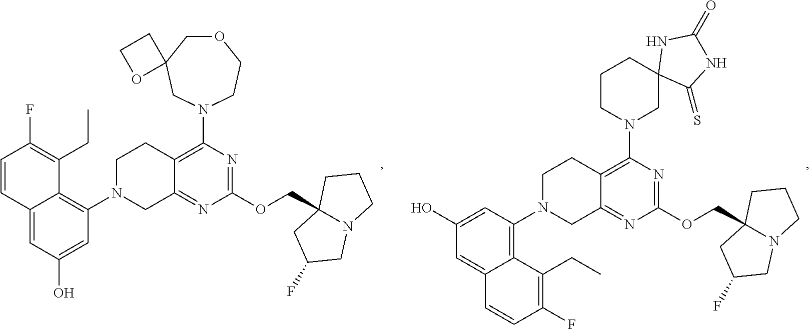 [SMILES: CCc1c(F)ccc2cc(O)cc(N3CCc4c(nc(OC[C@@]56CCCN5C[C@H](F)C6)nc4N4CCOCC5(CCO5)C4)C3)c12 ; CCc1c(F)ccc2cc(O)cc(N3CCc4c(nc(OC[C@@]56CCCN5C[C@H](F)C6)nc4N4CCCC5(C4)NC(=O)NC5=S)C3)c12] 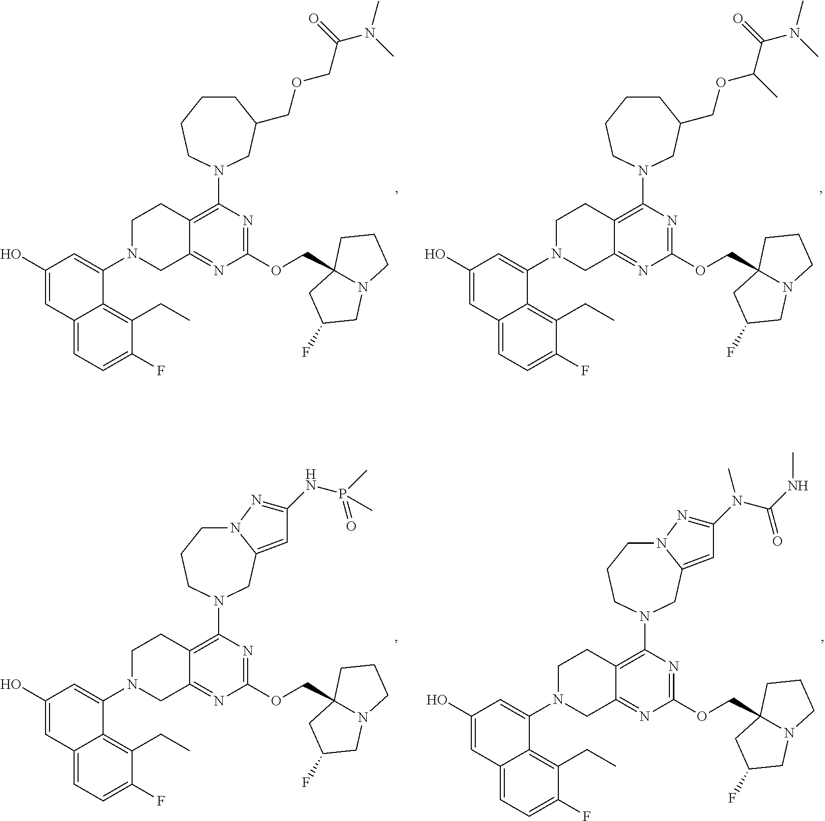 [SMILES: CCc1c(F)ccc2cc(O)cc(N3CCc4c(nc(OC[C@@]56CCCN5C[C@H](F)C6)nc4N4CCCCC(COCC(=O)N(C)C)C4)C3)c12 ; CCc1c(F)ccc2cc(O)cc(N3CCc4c(nc(OC[C@@]56CCCN5C[C@H](F)C6)nc4N4CCCCC(COC(C)C(=O)N(C)C)C4)C3)c12 ; CCc1c(F)ccc2cc(O)cc(N3CCc4c(nc(OC[C@@]56CCCN5C[C@H](F)C6)nc4N4CCCn5nc(NP(C)(C)=O)cc5C4)C3)c12 ; CCc1c(F)ccc2cc(O)cc(N3CCc4c(nc(OC[C@@]56CCCN5C[C@H](F)C6)nc4N4CCCn5nc(N(C)C(=O)NC)cc5C4)C3)c12] 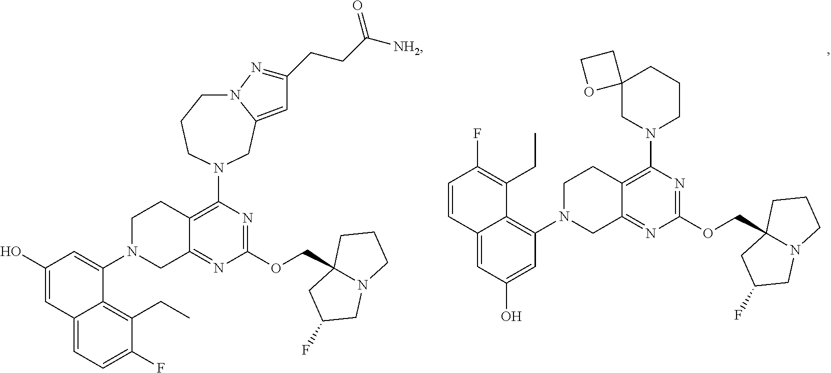 [SMILES: CCc1c(F)ccc2cc(O)cc(N3CCc4c(nc(OC[C@@]56CCCN5C[C@H](F)C6)nc4N4CCCn5nc(CCC(N)=O)cc5C4)C3)c12 ; CCc1c(F)ccc2cc(O)cc(N3CCc4c(nc(OC[C@@]56CCCN5C[C@H](F)C6)nc4N4CCCC5(CCO5)C4)C3)c12] 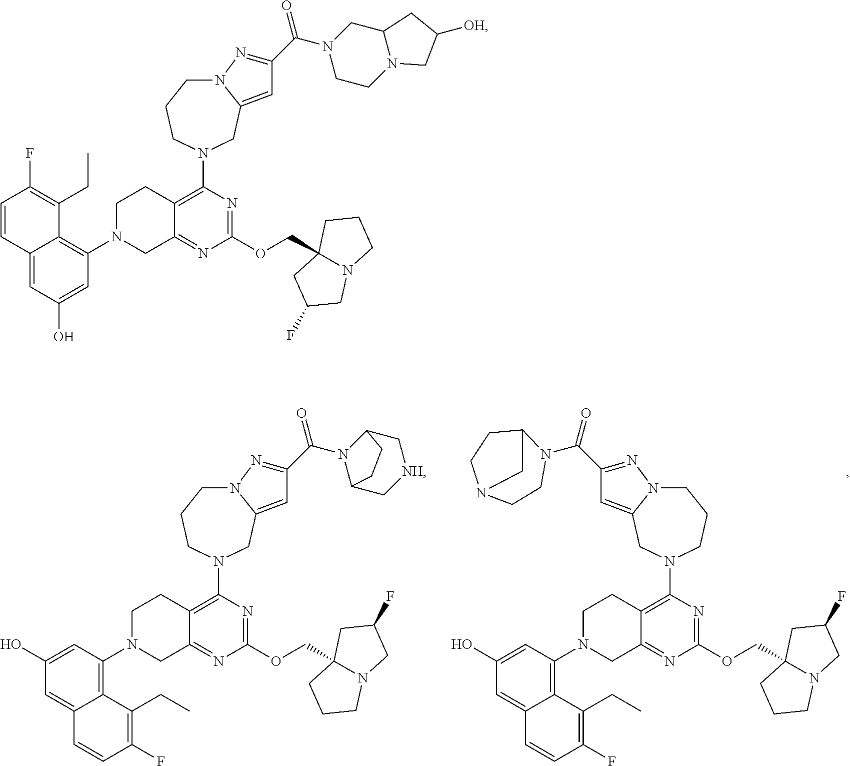 [SMILES: CCc1c(F)ccc2cc(O)cc(N3CCc4c(nc(OC[C@@]56CCCN5C[C@H](F)C6)nc4N4CCCn5nc(C(=O)N6CCN7CC(O)CC7C6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6C7CCC6CNC7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CCN7CCC6C7)cc5C4)C3)c12] 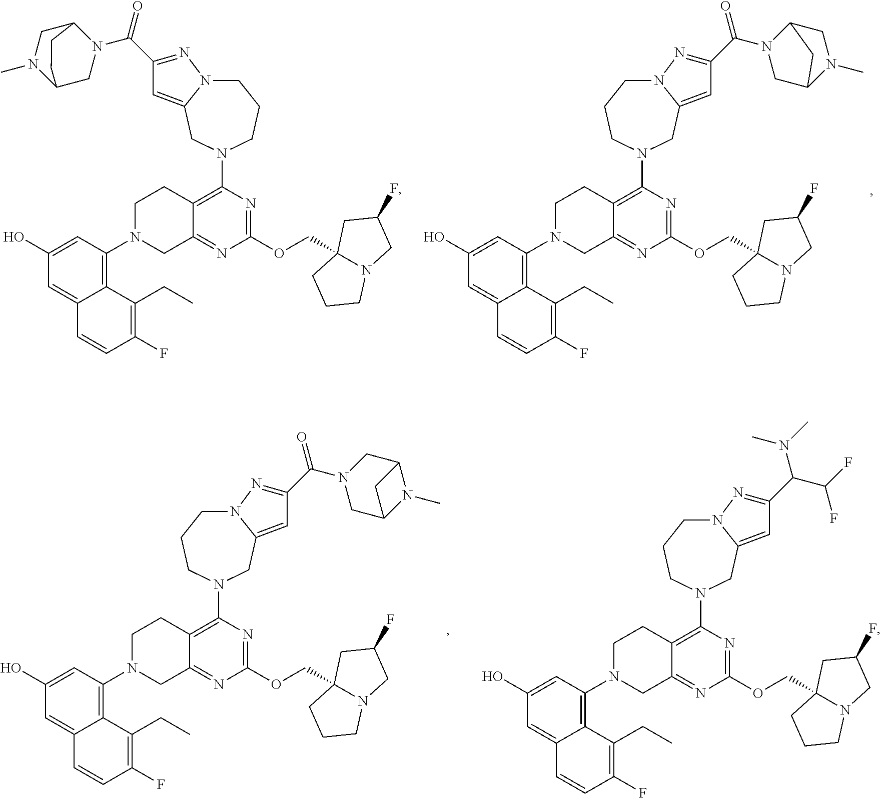 [SMILES: CCc1c(F)ccc2cc(O)cc(N3CCc4c(nc(OC[C@@]56CCCN5C[C@H](F)C6)nc4N4CCCn5nc(C(=O)N6CC7CCC6CN7C)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CC6CN7C)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CC(C6)N7C)cc5C4)C3)c12 ; CCc1c(F)ccc2cc(O)cc(N3CCc4c(nc(OC[C@@]56CCCN5C[C@H](F)C6)nc4N4CCCn5nc(C(C(F)F)N(C)C)cc5C4)C3)c12] 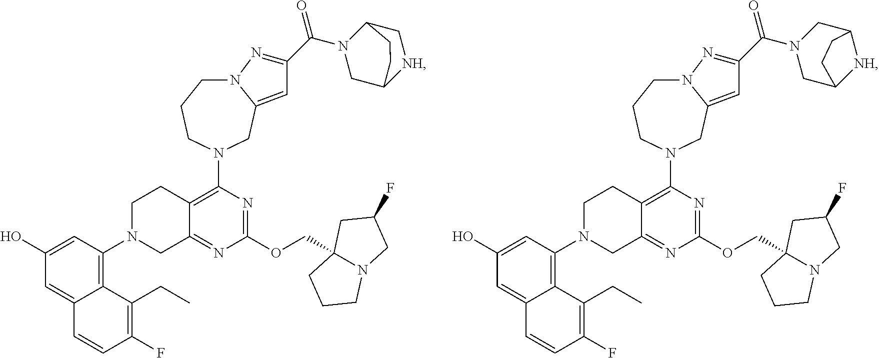 [SMILES: CCc1c(F)ccc2cc(O)cc(N3CCc4c(nc(OC[C@@]56CCCN5C[C@H](F)C6)nc4N4CCCn5nc(C(=O)N6CC7CCC6CN7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CCC(C6)N7)cc5C4)C3)c12] 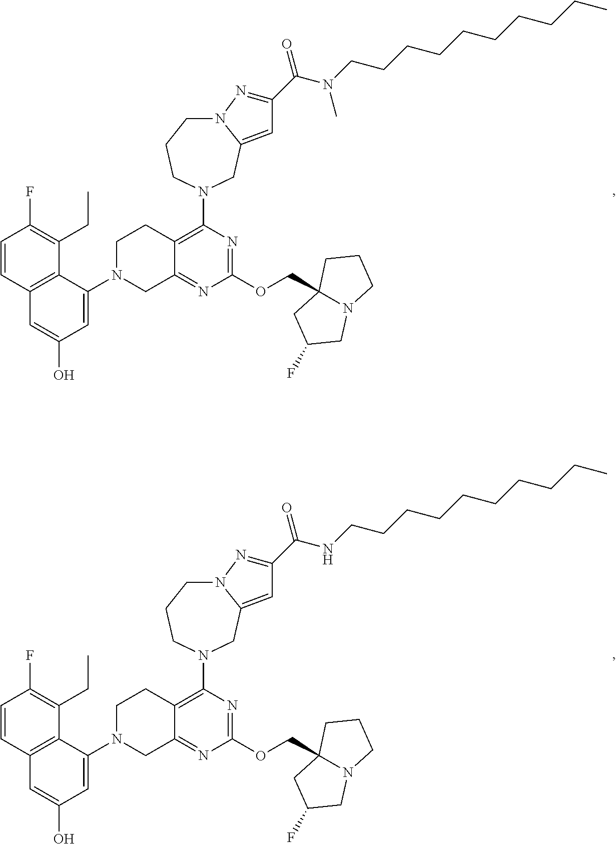 [SMILES: CCCCCCCCCCN(C)C(=O)c1cc2n(n1)CCCN(c1nc(OC[C@@]34CCCN3C[C@H](F)C4)nc3c1CCN(c1cc(O)cc4ccc(F)c(CC)c14)C3)C2 ; CCCCCCCCCCNC(=O)c1cc2n(n1)CCCN(c1nc(OC[C@@]34CCCN3C[C@H](F)C4)nc3c1CCN(c1cc(O)cc4ccc(F)c(CC)c14)C3)C2] 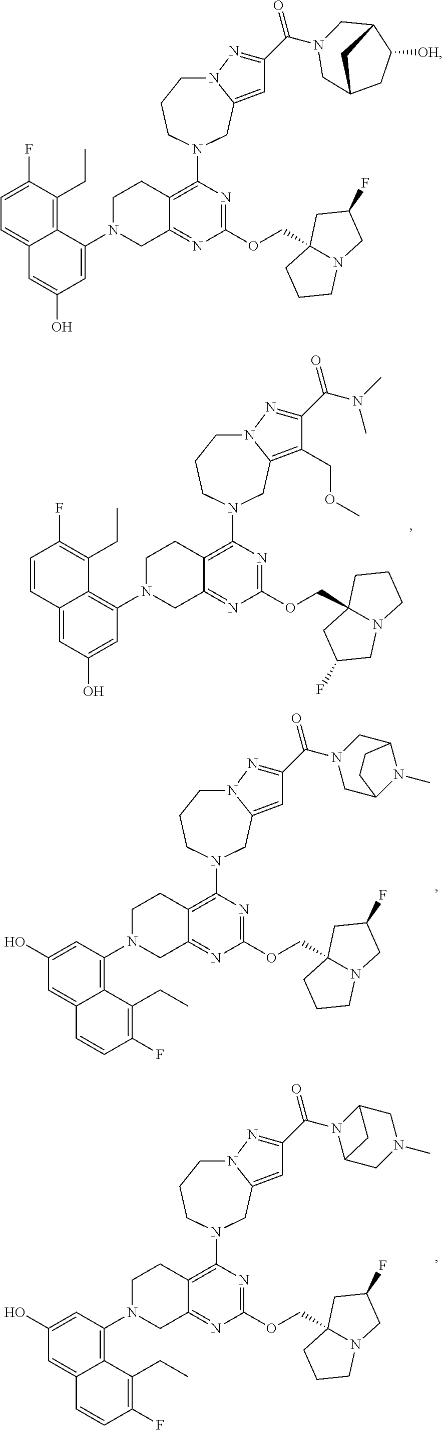 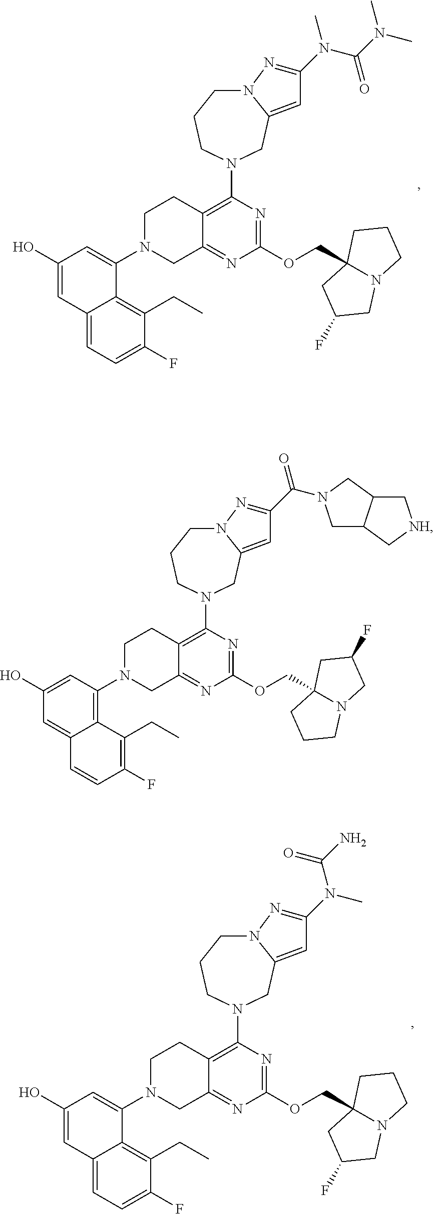 [SMILES: CCc1c(F)ccc2cc(O)cc(N3CCc4c(nc(OC[C@@]56CCCN5C[C@H](F)C6)nc4N4CCCn5nc(N(C)C(=O)N(C)C)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CNCC7C6)cc5C4)C3)c12 ; CCc1c(F)ccc2cc(O)cc(N3CCc4c(nc(OC[C@@]56CCCN5C[C@H](F)C6)nc4N4CCCn5nc(N(C)C(N)=O)cc5C4)C3)c12] 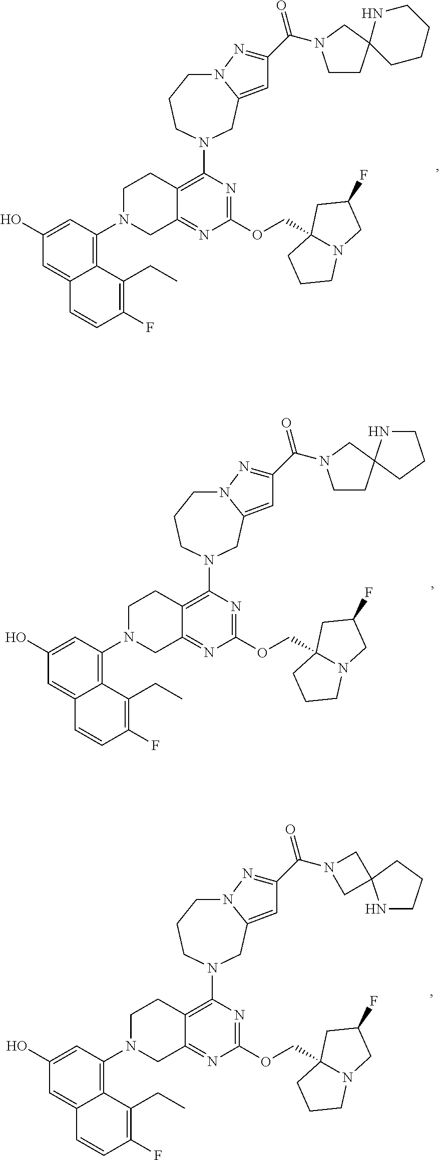 [SMILES: CCc1c(F)ccc2cc(O)cc(N3CCc4c(nc(OC[C@@]56CCCN5C[C@H](F)C6)nc4N4CCCn5nc(C(=O)N6CCC7(CCCCN7)C6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CCC7(CCCN7)C6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7(CCCN7)C6)cc5C4)C3)c12] 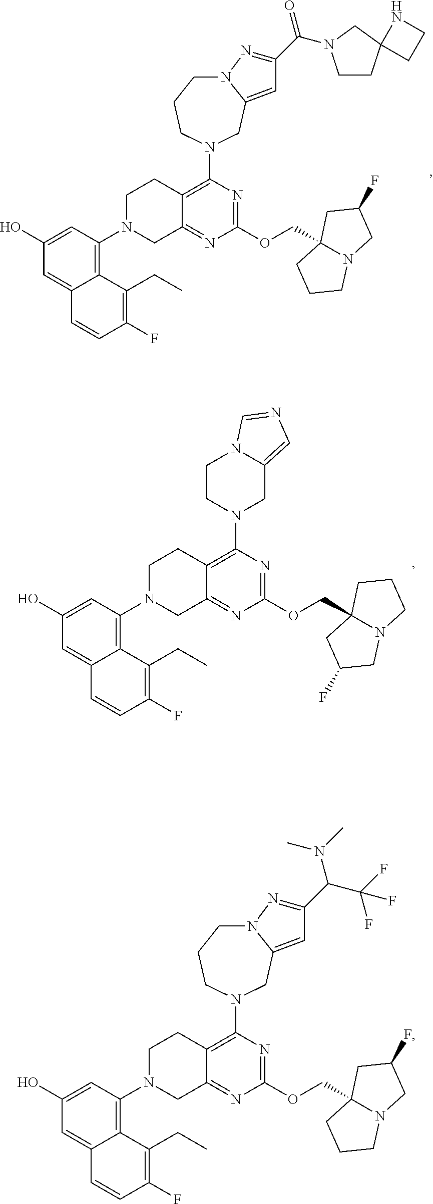 [SMILES: CCc1c(F)ccc2cc(O)cc(N3CCc4c(nc(OC[C@@]56CCCN5C[C@H](F)C6)nc4N4CCCn5nc(C(=O)N6CCC7(CCN7)C6)cc5C4)C3)c12 ; CCc1c(F)ccc2cc(O)cc(N3CCc4c(nc(OC[C@@]56CCCN5C[C@H](F)C6)nc4N4CCn5cncc5C4)C3)c12 ; CCc1c(F)ccc2cc(O)cc(N3CCc4c(nc(OC[C@@]56CCCN5C[C@H](F)C6)nc4N4CCCn5nc(C(N(C)C)C(F)(F)F)cc5C4)C3)c12] 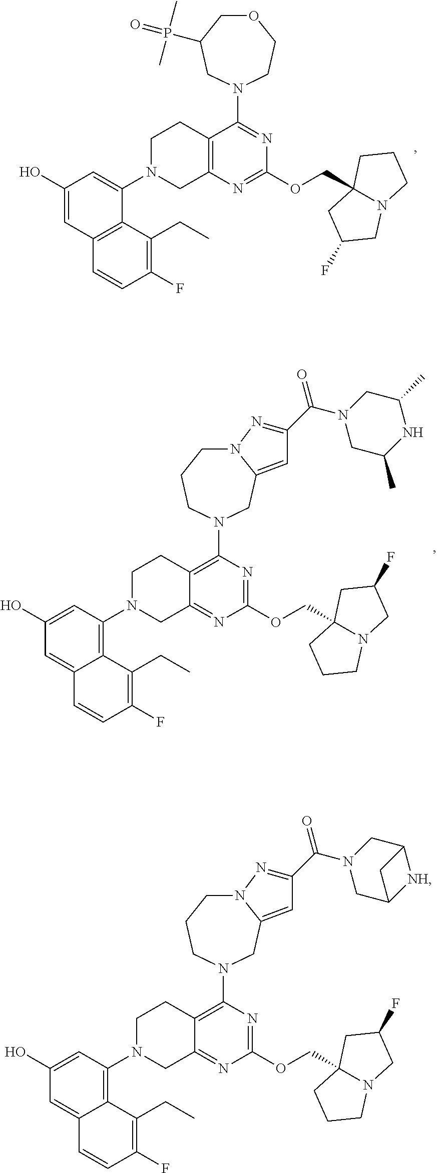 [SMILES: CCc1c(F)ccc2cc(O)cc(N3CCc4c(nc(OC[C@@]56CCCN5C[C@H](F)C6)nc4N4CCOCC(P(C)(C)=O)C4)C3)c12 ; CCc1c(F)ccc2cc(O)cc(N3CCc4c(nc(OC[C@@]56CCCN5C[C@H](F)C6)nc4N4CCCn5nc(C(=O)N6C[C@H](C)N[C@@H](C)C6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CC(C6)N7)cc5C4)C3)c12] 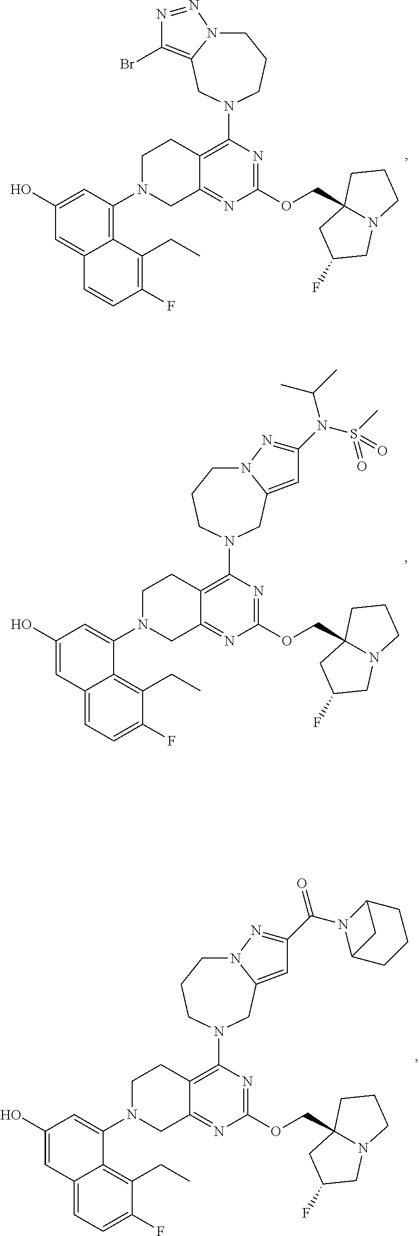 [SMILES: CCc1c(F)ccc2cc(O)cc(N3CCc4c(nc(OC[C@@]56CCCN5C[C@H](F)C6)nc4N4CCCn5nnc(Br)c5C4)C3)c12 ; CCc1c(F)ccc2cc(O)cc(N3CCc4c(nc(OC[C@@]56CCCN5C[C@H](F)C6)nc4N4CCCn5nc(N(C(C)C)S(C)(=O)=O)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6C7CCCC6C7)cc5C4)C3)c12] 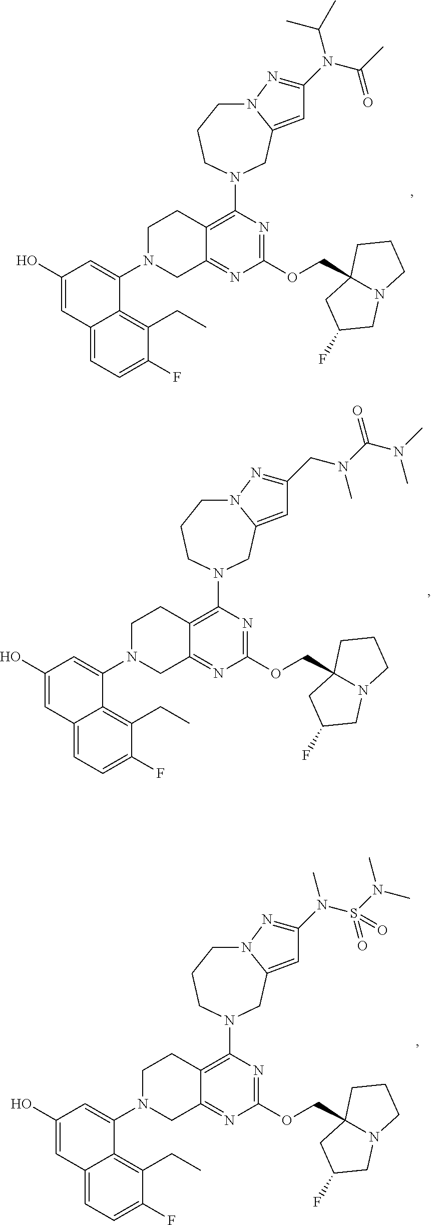 [SMILES: CCc1c(F)ccc2cc(O)cc(N3CCc4c(nc(OC[C@@]56CCCN5C[C@H](F)C6)nc4N4CCCn5nc(N(C(C)=O)C(C)C)cc5C4)C3)c12 ; CCc1c(F)ccc2cc(O)cc(N3CCc4c(nc(OC[C@@]56CCCN5C[C@H](F)C6)nc4N4CCCn5nc(CN(C)C(=O)N(C)C)cc5C4)C3)c12 ; CCc1c(F)ccc2cc(O)cc(N3CCc4c(nc(OC[C@@]56CCCN5C[C@H](F)C6)nc4N4CCCn5nc(N(C)S(=O)(=O)N(C)C)cc5C4)C3)c12] 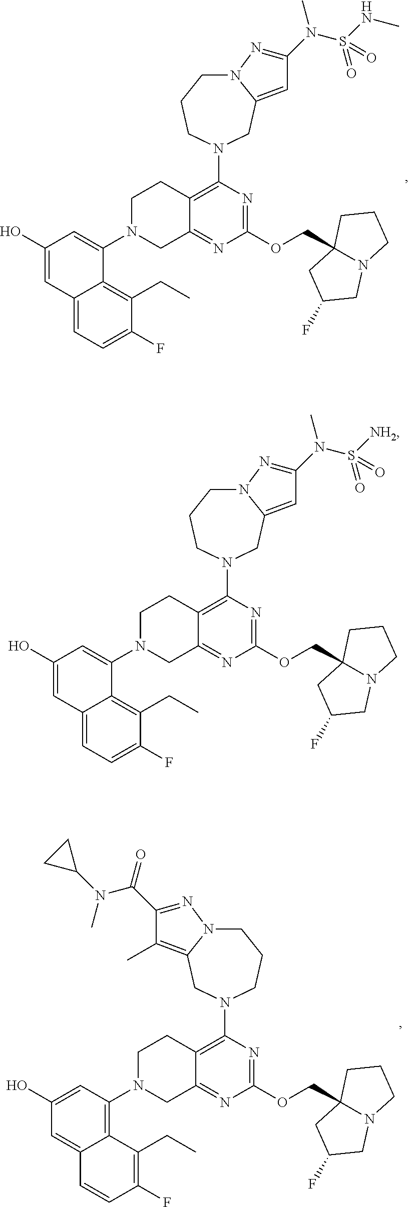 [SMILES: CCc1c(F)ccc2cc(O)cc(N3CCc4c(nc(OC[C@@]56CCCN5C[C@H](F)C6)nc4N4CCCn5nc(N(C)S(=O)(=O)NC)cc5C4)C3)c12 ; CCc1c(F)ccc2cc(O)cc(N3CCc4c(nc(OC[C@@]56CCCN5C[C@H](F)C6)nc4N4CCCn5nc(N(C)S(N)(=O)=O)cc5C4)C3)c12 ; CCc1c(F)ccc2cc(O)cc(N3CCc4c(nc(OC[C@@]56CCCN5C[C@H](F)C6)nc4N4CCCn5nc(C(=O)N(C)C6CC6)c(C)c5C4)C3)c12] 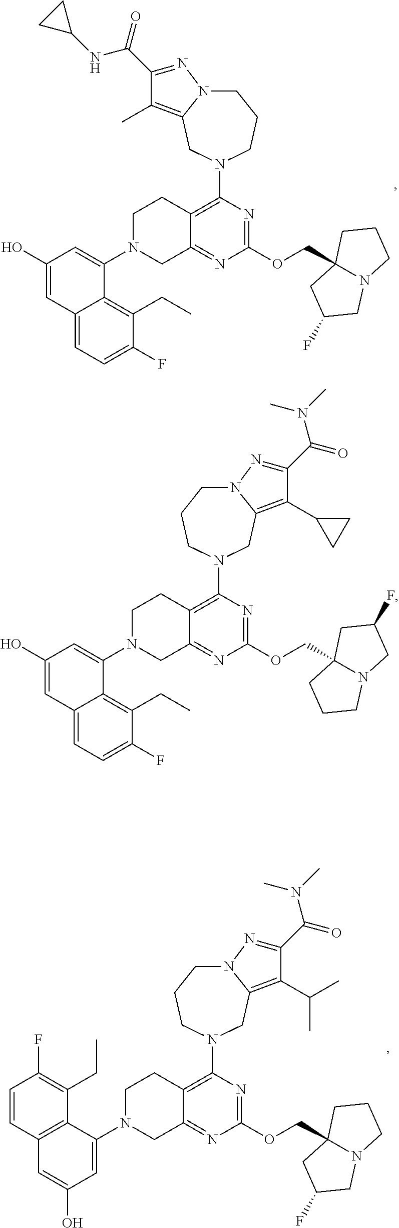 [SMILES: CCc1c(F)ccc2cc(O)cc(N3CCc4c(nc(OC[C@@]56CCCN5C[C@H](F)C6)nc4N4CCCn5nc(C(=O)NC6CC6)c(C)c5C4)C3)c12 ; CCc1c(F)ccc2cc(O)cc(N3CCc4c(nc(OC[C@@]56CCCN5C[C@H](F)C6)nc4N4CCCn5nc(C(=O)N(C)C)c(C6CC6)c5C4)C3)c12 ; CCc1c(F)ccc2cc(O)cc(N3CCc4c(nc(OC[C@@]56CCCN5C[C@H](F)C6)nc4N4CCCn5nc(C(=O)N(C)C)c(C(C)C)c5C4)C3)c12] 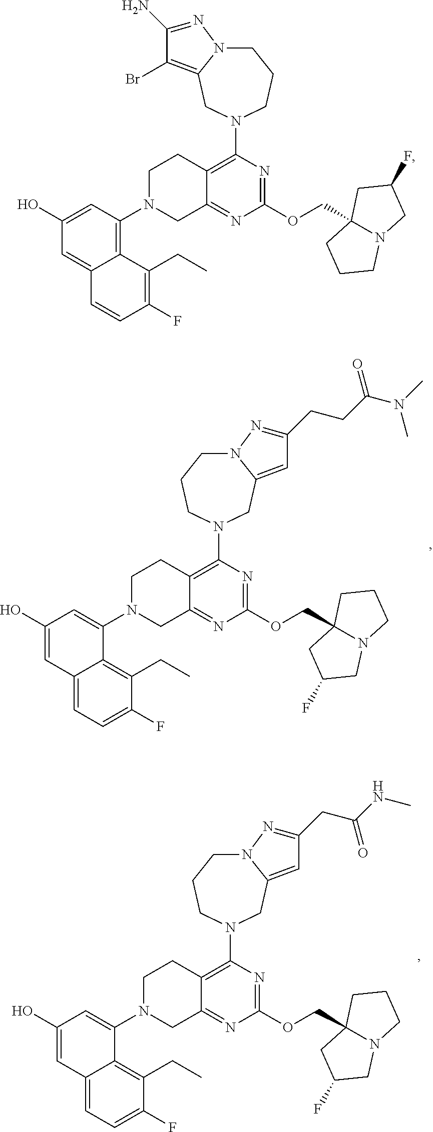 [SMILES: CCc1c(F)ccc2cc(O)cc(N3CCc4c(nc(OC[C@@]56CCCN5C[C@H](F)C6)nc4N4CCCn5nc(N)c(Br)c5C4)C3)c12 ; CCc1c(F)ccc2cc(O)cc(N3CCc4c(nc(OC[C@@]56CCCN5C[C@H](F)C6)nc4N4CCCn5nc(CCC(=O)N(C)C)cc5C4)C3)c12 ; CCc1c(F)ccc2cc(O)cc(N3CCc4c(nc(OC[C@@]56CCCN5C[C@H](F)C6)nc4N4CCCn5nc(CC(=O)NC)cc5C4)C3)c12] 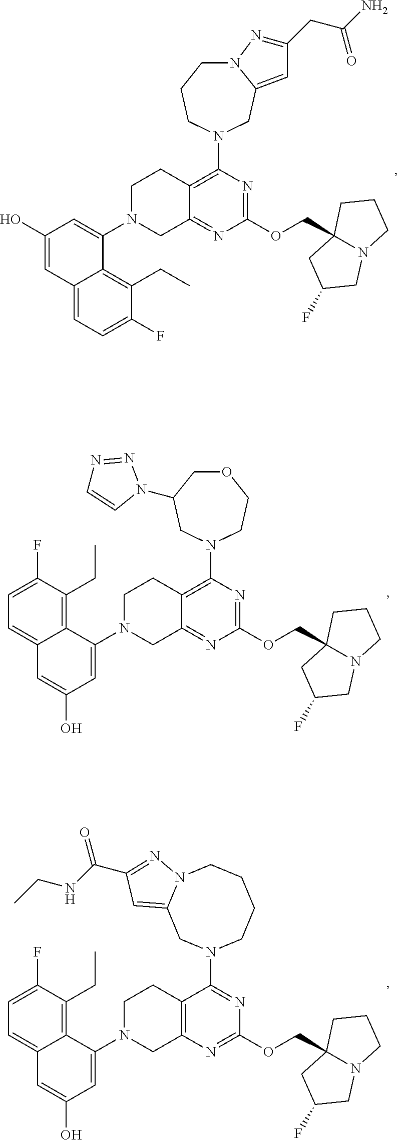 [SMILES: CCc1c(F)ccc2cc(O)cc(N3CCc4c(nc(OC[C@@]56CCCN5C[C@H](F)C6)nc4N4CCCn5nc(CC(N)=O)cc5C4)C3)c12 ; CCc1c(F)ccc2cc(O)cc(N3CCc4c(nc(OC[C@@]56CCCN5C[C@H](F)C6)nc4N4CCOCC(n5ccnn5)C4)C3)c12 ; CCNC(=O)c1cc2n(n1)CCCCN(c1nc(OC[C@@]34CCCN3C[C@H](F)C4)nc3c1CCN(c1cc(O)cc4ccc(F)c(CC)c14)C3)C2] 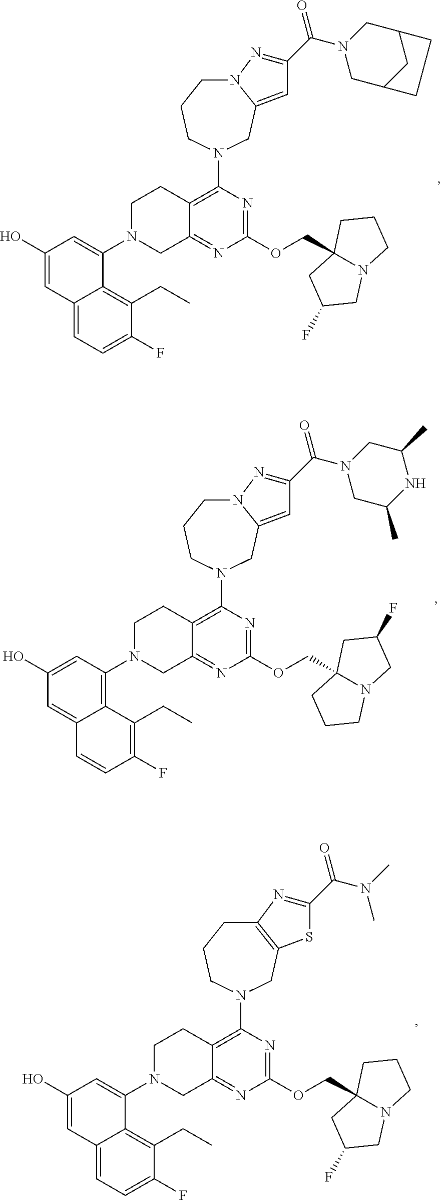 [SMILES: CCc1c(F)ccc2cc(O)cc(N3CCc4c(nc(OC[C@@]56CCCN5C[C@H](F)C6)nc4N4CCCn5nc(C(=O)N6CC7CCC(C7)C6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6C[C@@H](C)N[C@@H](C)C6)cc5C4)C3)c12 ; CCc1c(F)ccc2cc(O)cc(N3CCc4c(nc(OC[C@@]56CCCN5C[C@H](F)C6)nc4N4CCCc5nc(C(=O)N(C)C)sc5C4)C3)c12] 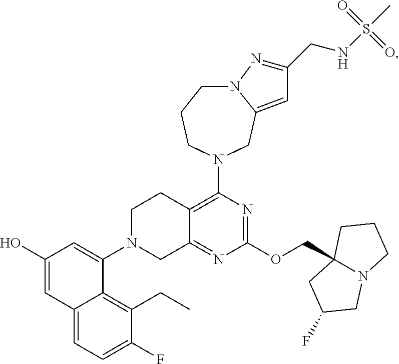 [SMILES: CCc1c(F)ccc2cc(O)cc(N3CCc4c(nc(OC[C@@]56CCCN5C[C@H](F)C6)nc4N4CCCn5nc(CNS(C)(=O)=O)cc5C4)C3)c12] 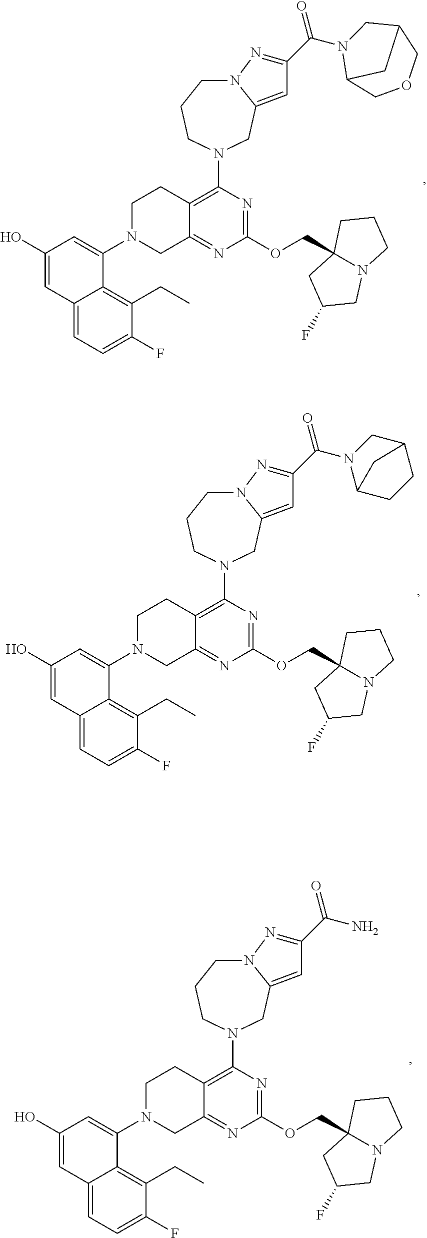 [SMILES: CCc1c(F)ccc2cc(O)cc(N3CCc4c(nc(OC[C@@]56CCCN5C[C@H](F)C6)nc4N4CCCn5nc(C(=O)N6CC7COCC6C7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CCC6C7)cc5C4)C3)c12 ; CCc1c(F)ccc2cc(O)cc(N3CCc4c(nc(OC[C@@]56CCCN5C[C@H](F)C6)nc4N4CCCn5nc(C(N)=O)cc5C4)C3)c12] 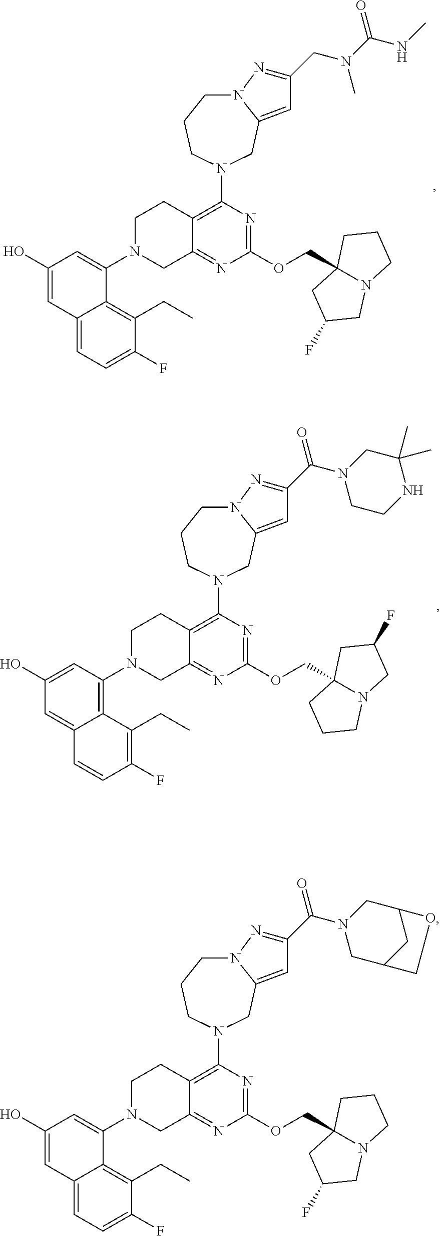 [SMILES: CCc1c(F)ccc2cc(O)cc(N3CCc4c(nc(OC[C@@]56CCCN5C[C@H](F)C6)nc4N4CCCn5nc(CN(C)C(=O)NC)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CCNC(C)(C)C6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7COC(C7)C6)cc5C4)C3)c12] 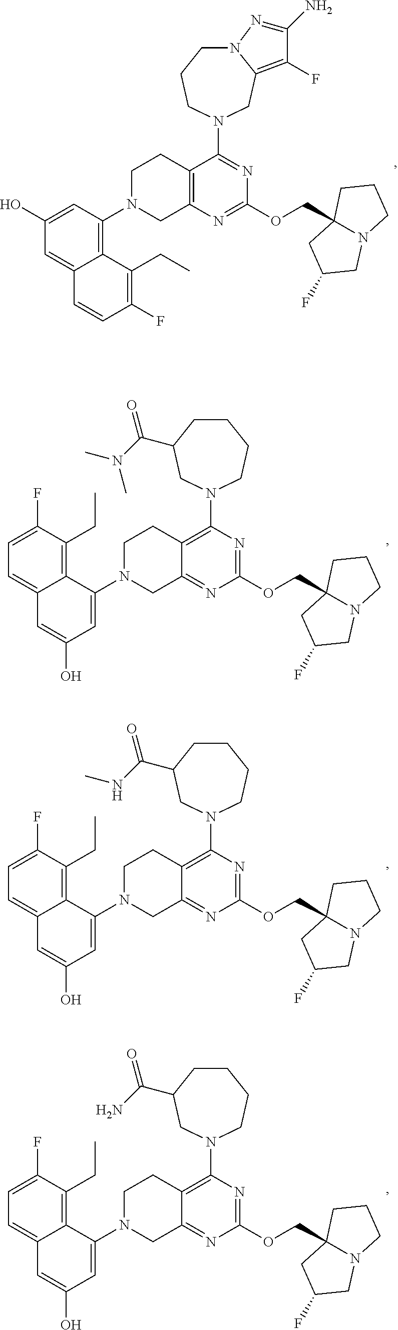 [SMILES: CCc1c(F)ccc2cc(O)cc(N3CCc4c(nc(OC[C@@]56CCCN5C[C@H](F)C6)nc4N4CCCn5nc(N)c(F)c5C4)C3)c12 ; CCc1c(F)ccc2cc(O)cc(N3CCc4c(nc(OC[C@@]56CCCN5C[C@H](F)C6)nc4N4CCCCC(C(=O)N(C)C)C4)C3)c12 ; CCc1c(F)ccc2cc(O)cc(N3CCc4c(nc(OC[C@@]56CCCN5C[C@H](F)C6)nc4N4CCCCC(C(=O)NC)C4)C3)c12 ; CCc1c(F)ccc2cc(O)cc(N3CCc4c(nc(OC[C@@]56CCCN5C[C@H](F)C6)nc4N4CCCCC(C(N)=O)C4)C3)c12] 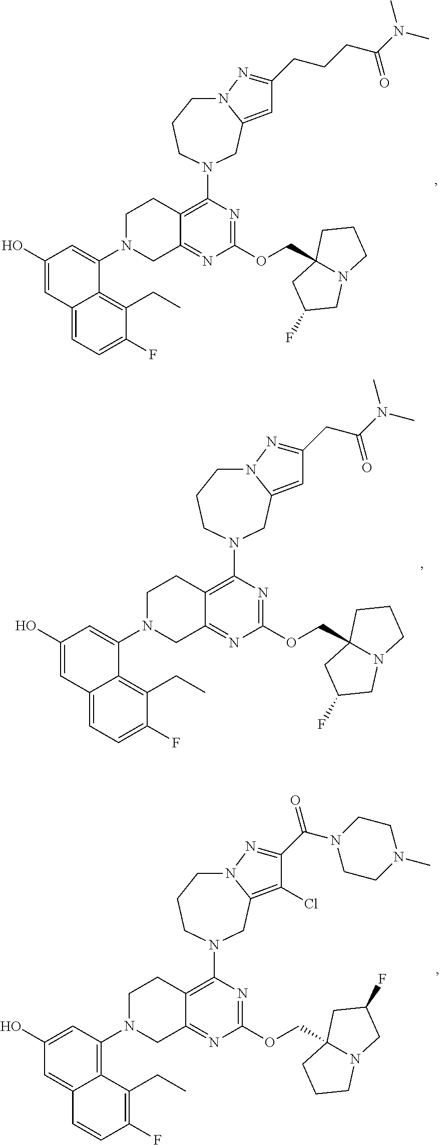 [SMILES: CCc1c(F)ccc2cc(O)cc(N3CCc4c(nc(OC[C@@]56CCCN5C[C@H](F)C6)nc4N4CCCn5nc(CCCC(=O)N(C)C)cc5C4)C3)c12 ; CCc1c(F)ccc2cc(O)cc(N3CCc4c(nc(OC[C@@]56CCCN5C[C@H](F)C6)nc4N4CCCn5nc(CC(=O)N(C)C)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CCN(C)CC6)c(Cl)c5C4)C3)c12] 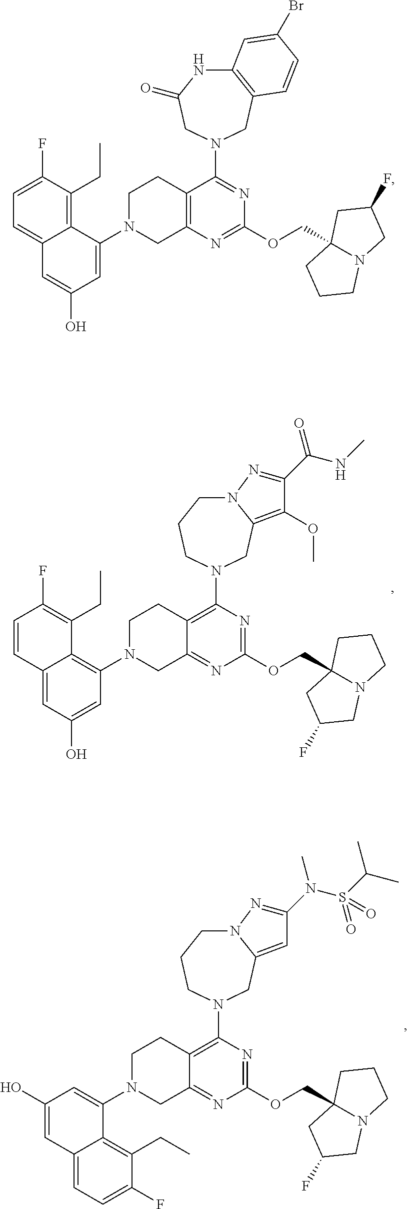 [SMILES: CCc1c(F)ccc2cc(O)cc(N3CCc4c(nc(OC[C@@]56CCCN5C[C@H](F)C6)nc4N4CC(=O)Nc5cc(Br)ccc5C4)C3)c12 ; CCc1c(F)ccc2cc(O)cc(N3CCc4c(nc(OC[C@@]56CCCN5C[C@H](F)C6)nc4N4CCCn5nc(C(=O)NC)c(OC)c5C4)C3)c12 ; CCc1c(F)ccc2cc(O)cc(N3CCc4c(nc(OC[C@@]56CCCN5C[C@H](F)C6)nc4N4CCCn5nc(N(C)S(=O)(=O)C(C)C)cc5C4)C3)c12] 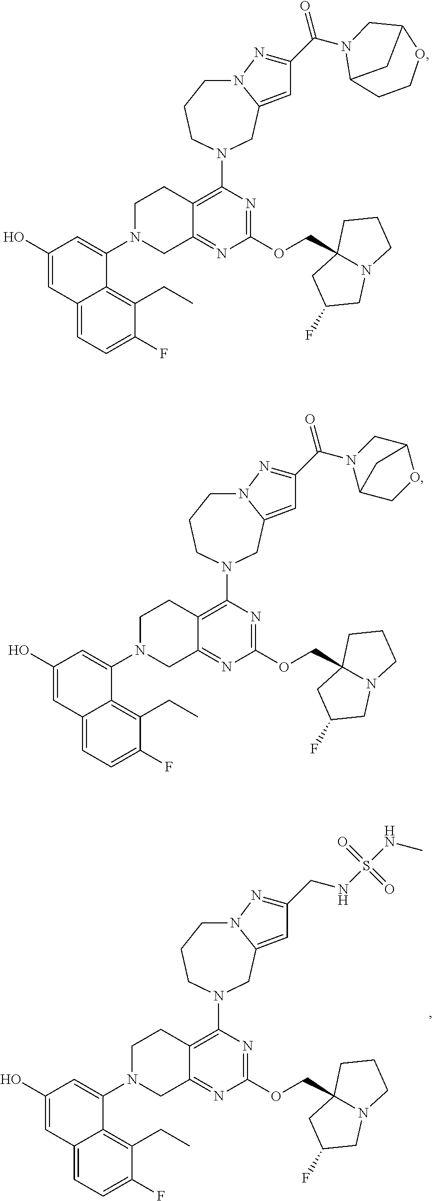 [SMILES: CCc1c(F)ccc2cc(O)cc(N3CCc4c(nc(OC[C@@]56CCCN5C[C@H](F)C6)nc4N4CCCn5nc(C(=O)N6CC7CC6CCO7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CC6CO7)cc5C4)C3)c12 ; CCc1c(F)ccc2cc(O)cc(N3CCc4c(nc(OC[C@@]56CCCN5C[C@H](F)C6)nc4N4CCCn5nc(CNS(=O)(=O)NC)cc5C4)C3)c12] 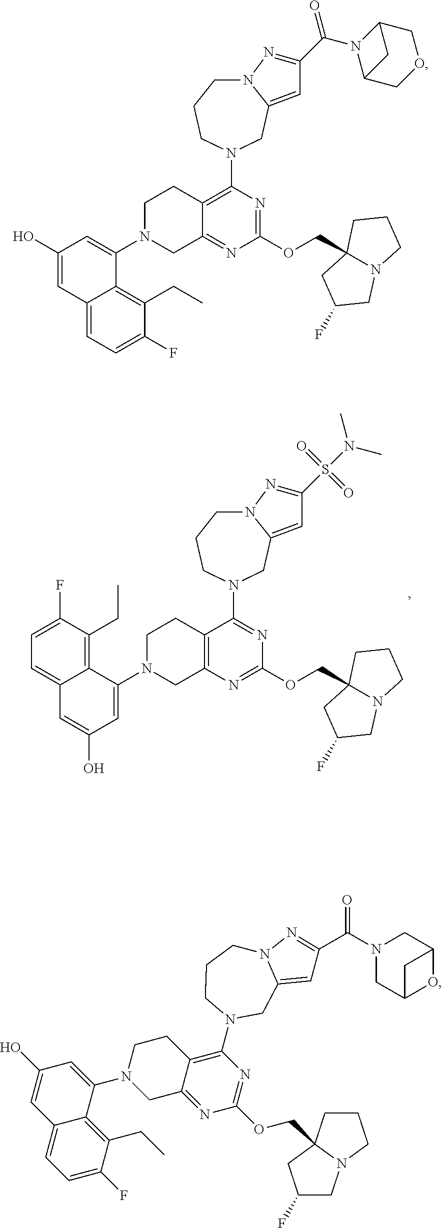 [SMILES: CCc1c(F)ccc2cc(O)cc(N3CCc4c(nc(OC[C@@]56CCCN5C[C@H](F)C6)nc4N4CCCn5nc(C(=O)N6C7COCC6C7)cc5C4)C3)c12 ; CCc1c(F)ccc2cc(O)cc(N3CCc4c(nc(OC[C@@]56CCCN5C[C@H](F)C6)nc4N4CCCn5nc(S(=O)(=O)N(C)C)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CC(C6)O7)cc5C4)C3)c12] 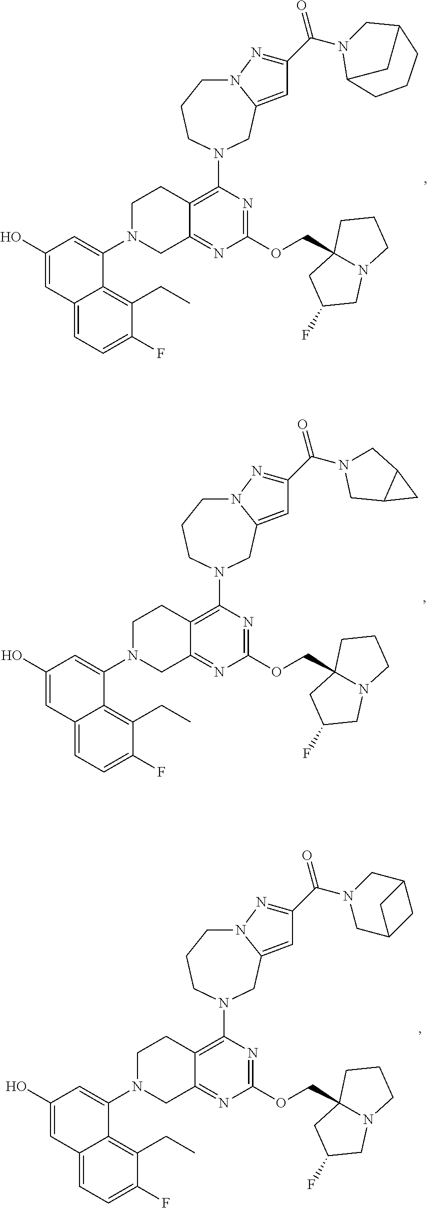 [SMILES: CCc1c(F)ccc2cc(O)cc(N3CCc4c(nc(OC[C@@]56CCCN5C[C@H](F)C6)nc4N4CCCn5nc(C(=O)N6CC7CCCC6C7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CC7C6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CC(C7)C6)cc5C4)C3)c12] 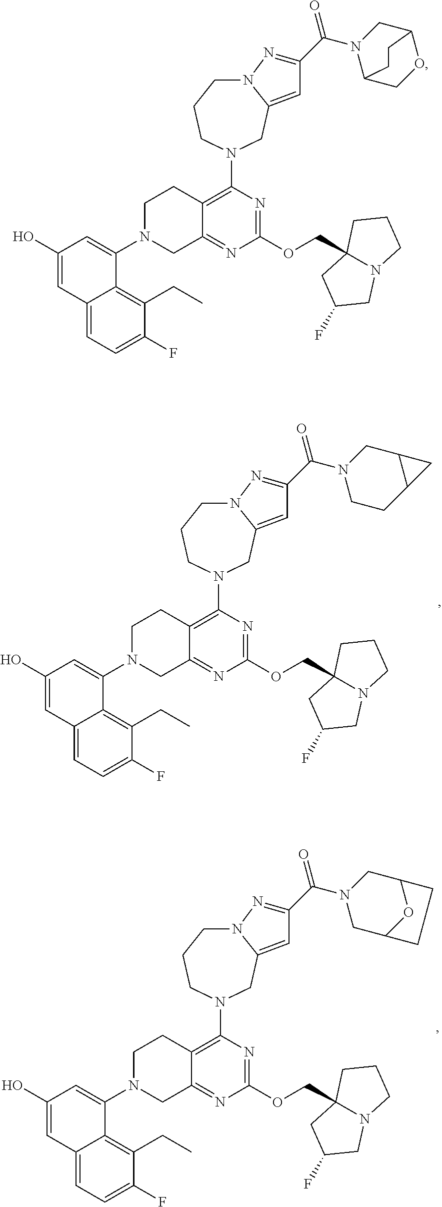 [SMILES: CCc1c(F)ccc2cc(O)cc(N3CCc4c(nc(OC[C@@]56CCCN5C[C@H](F)C6)nc4N4CCCn5nc(C(=O)N6CC7CCC6CO7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CCC7CC7C6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CCC(C6)O7)cc5C4)C3)c12] 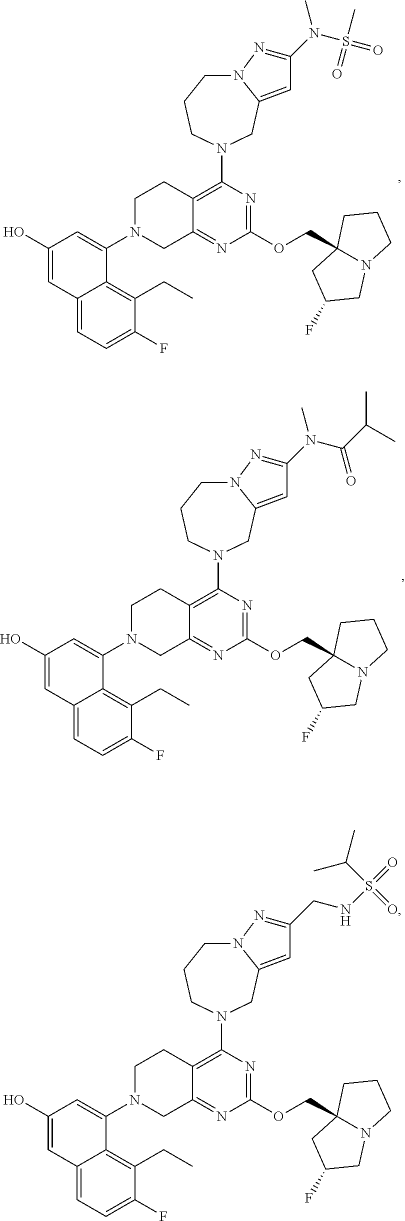 [SMILES: CCc1c(F)ccc2cc(O)cc(N3CCc4c(nc(OC[C@@]56CCCN5C[C@H](F)C6)nc4N4CCCn5nc(N(C)S(C)(=O)=O)cc5C4)C3)c12 ; CCc1c(F)ccc2cc(O)cc(N3CCc4c(nc(OC[C@@]56CCCN5C[C@H](F)C6)nc4N4CCCn5nc(N(C)C(=O)C(C)C)cc5C4)C3)c12 ; CCc1c(F)ccc2cc(O)cc(N3CCc4c(nc(OC[C@@]56CCCN5C[C@H](F)C6)nc4N4CCCn5nc(CNS(=O)(=O)C(C)C)cc5C4)C3)c12] 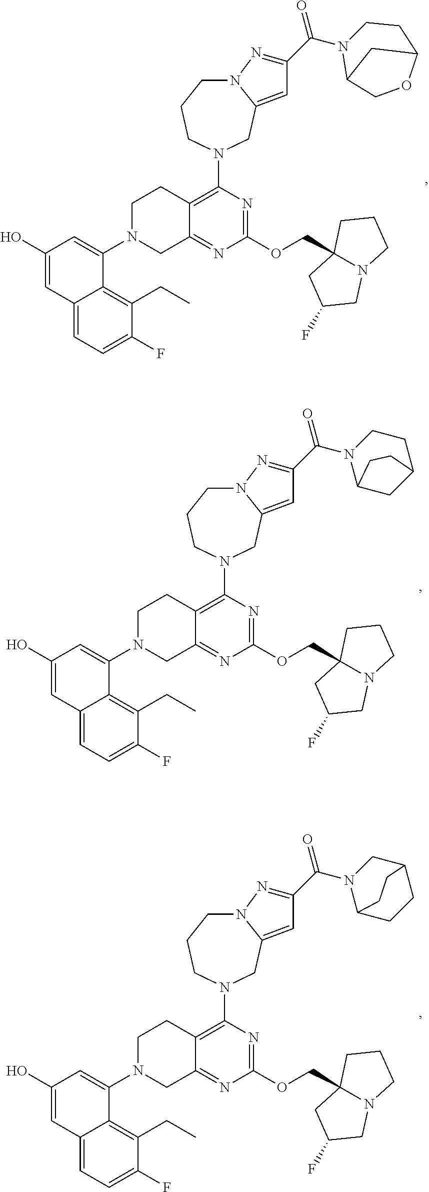 [SMILES: CCc1c(F)ccc2cc(O)cc(N3CCc4c(nc(OC[C@@]56CCCN5C[C@H](F)C6)nc4N4CCCn5nc(C(=O)N6CCC7CC6CO7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CCC7CCC6C7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CCC6CC7)cc5C4)C3)c12] 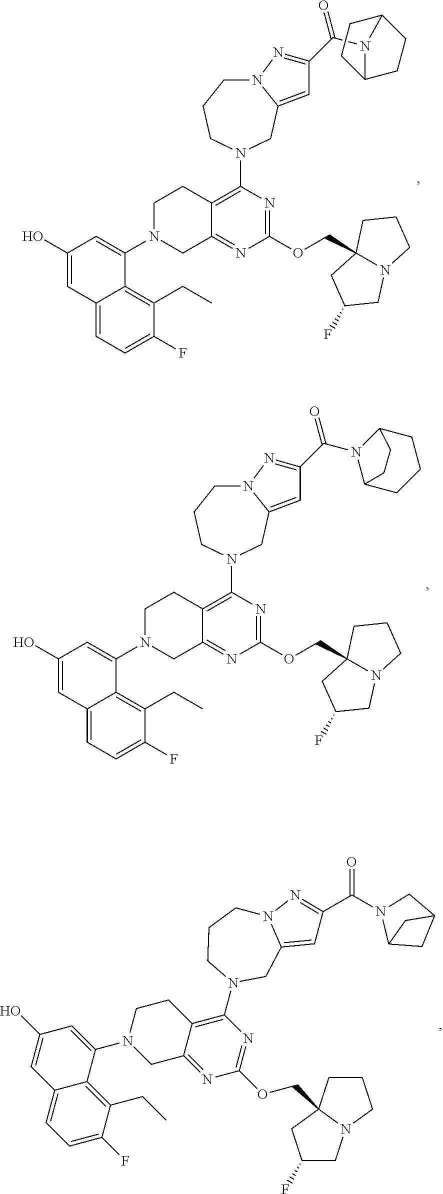 [SMILES: CCc1c(F)ccc2cc(O)cc(N3CCc4c(nc(OC[C@@]56CCCN5C[C@H](F)C6)nc4N4CCCn5nc(C(=O)N6C7CCC6CC7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6C7CCCC6CC7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7CC6C7)cc5C4)C3)c12] 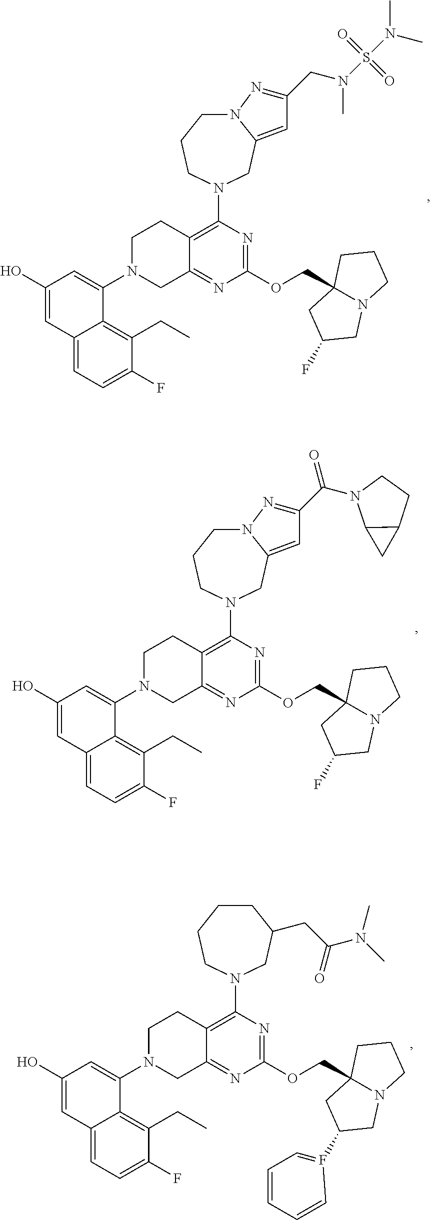 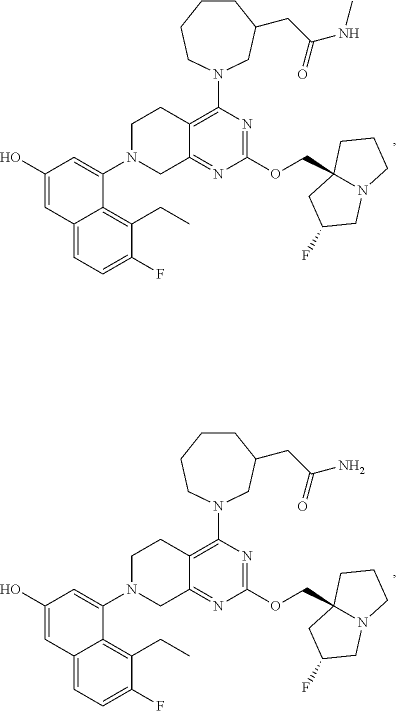 [SMILES: CCc1c(F)ccc2cc(O)cc(N3CCc4c(nc(OC[C@@]56CCCN5C[C@H](F)C6)nc4N4CCCCC(CC(=O)NC)C4)C3)c12 ; CCc1c(F)ccc2cc(O)cc(N3CCc4c(nc(OC[C@@]56CCCN5C[C@H](F)C6)nc4N4CCCCC(CC(N)=O)C4)C3)c12] 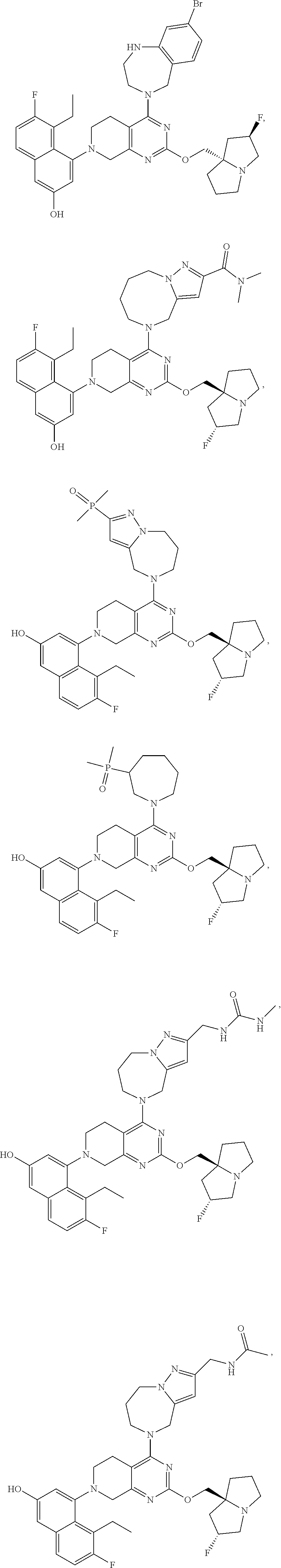 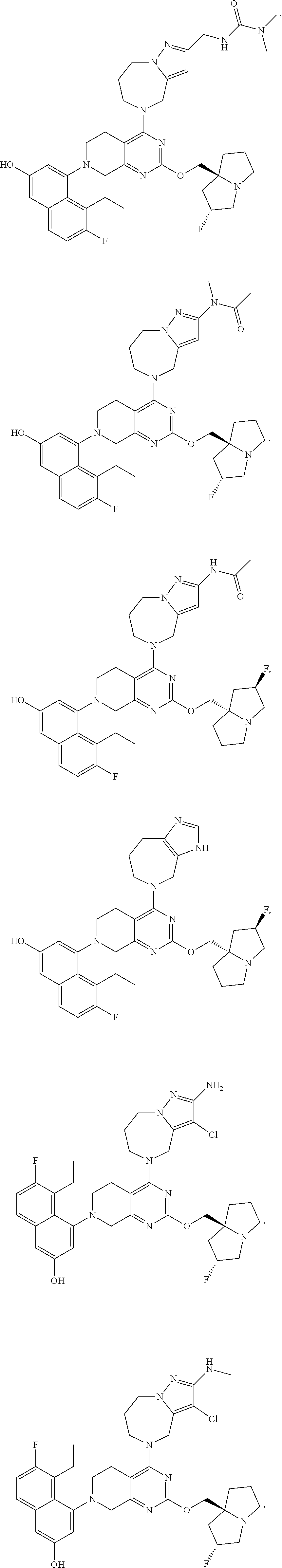 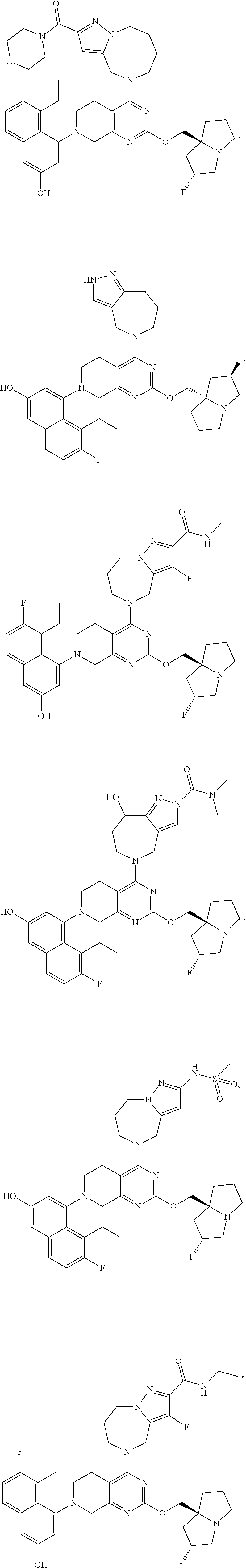 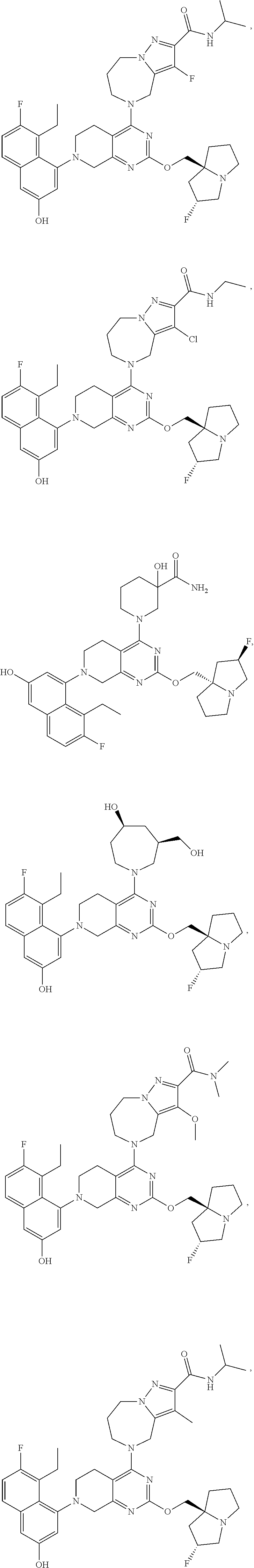 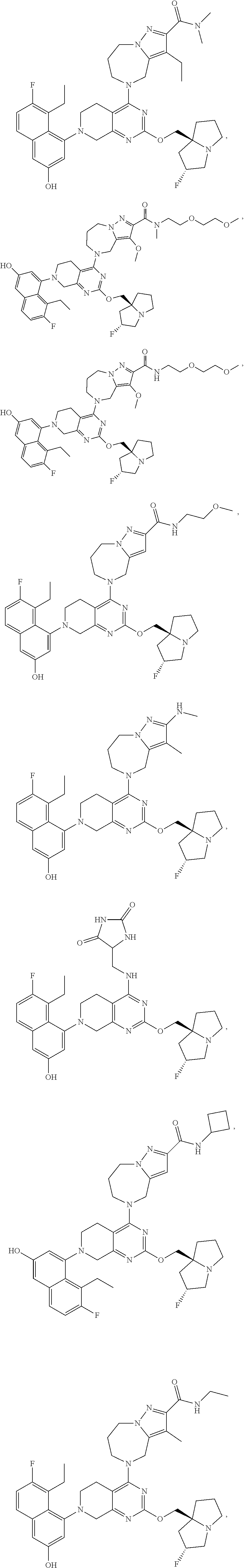 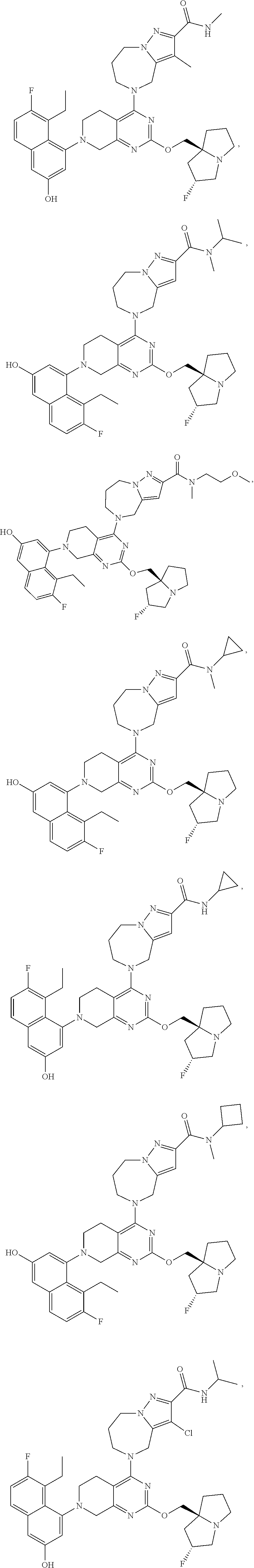 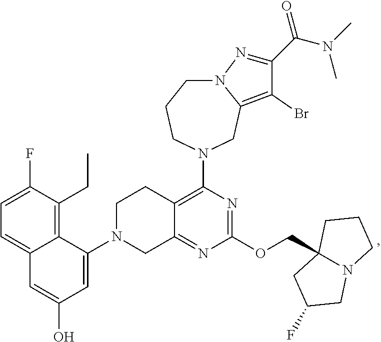 [SMILES: CCc1c(F)ccc2cc(O)cc(N3CCc4c(nc(OC[C@@]56CCCN5C[C@H](F)C6)nc4N4CCCn5nc(C(=O)N(C)C)c(Br)c5C4)C3)c12] 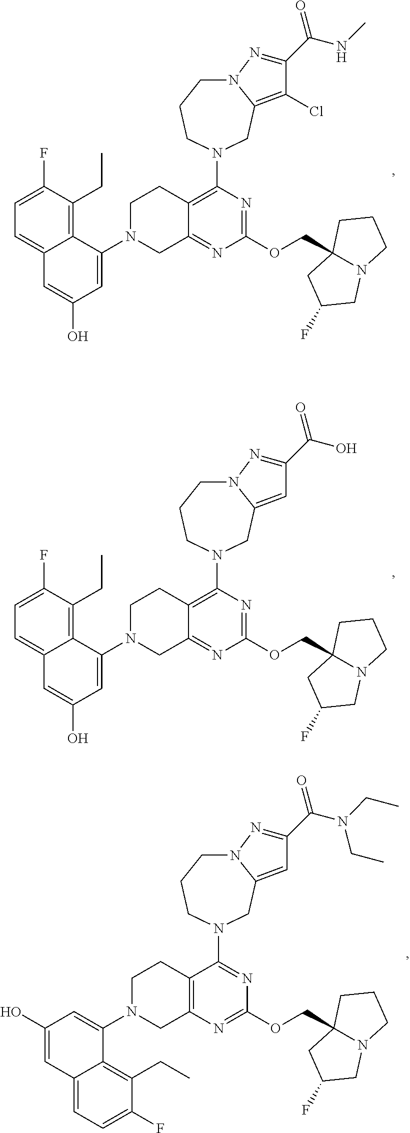 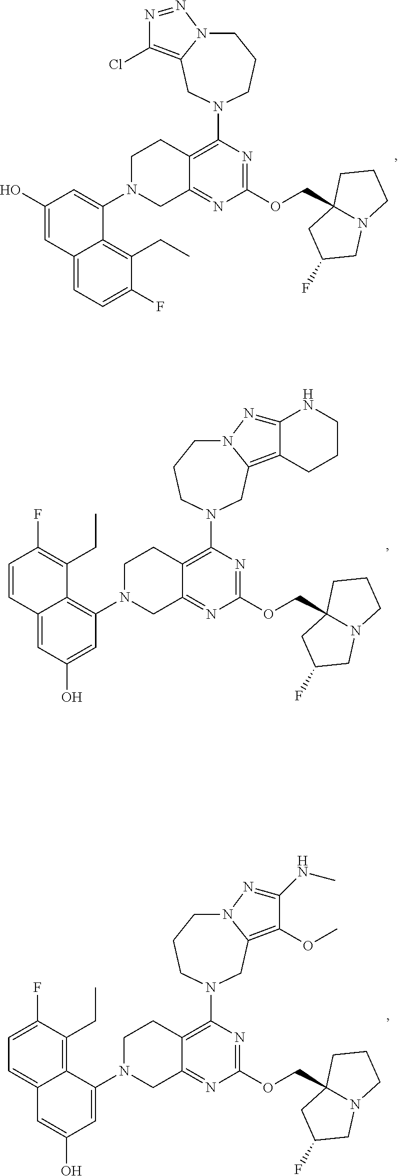 [SMILES: CCc1c(F)ccc2cc(O)cc(N3CCc4c(nc(OC[C@@]56CCCN5C[C@H](F)C6)nc4N4CCCn5nnc(Cl)c5C4)C3)c12 ; CCc1c(F)ccc2cc(O)cc(N3CCc4c(nc(OC[C@@]56CCCN5C[C@H](F)C6)nc4N4CCCn5nc6c(c5C4)CCCN6)C3)c12 ; CCc1c(F)ccc2cc(O)cc(N3CCc4c(nc(OC[C@@]56CCCN5C[C@H](F)C6)nc4N4CCCn5nc(NC)c(OC)c5C4)C3)c12] 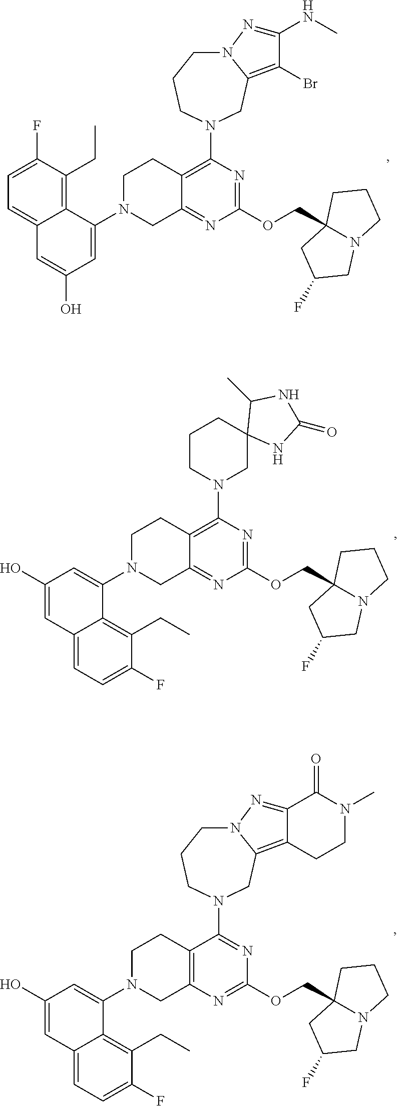 [SMILES: CCc1c(F)ccc2cc(O)cc(N3CCc4c(nc(OC[C@@]56CCCN5C[C@H](F)C6)nc4N4CCCn5nc(NC)c(Br)c5C4)C3)c12 ; CCc1c(F)ccc2cc(O)cc(N3CCc4c(nc(OC[C@@]56CCCN5C[C@H](F)C6)nc4N4CCCC5(C4)NC(=O)NC5C)C3)c12 ; CCc1c(F)ccc2cc(O)cc(N3CCc4c(nc(OC[C@@]56CCCN5C[C@H](F)C6)nc4N4CCCn5nc6c(c5C4)CCN(C)C6=O)C3)c12] 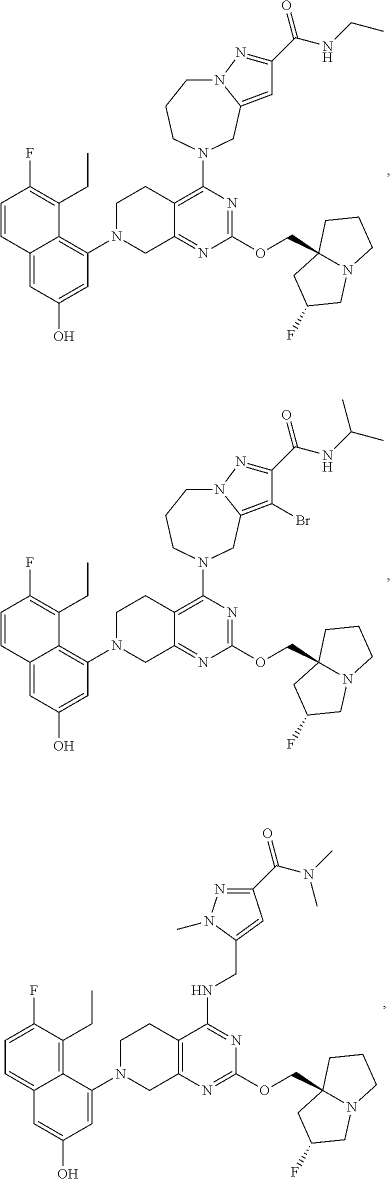 [SMILES: CCNC(=O)c1cc2n(n1)CCCN(c1nc(OC[C@@]34CCCN3C[C@H](F)C4)nc3c1CCN(c1cc(O)cc4ccc(F)c(CC)c14)C3)C2 ; CCc1c(F)ccc2cc(O)cc(N3CCc4c(nc(OC[C@@]56CCCN5C[C@H](F)C6)nc4N4CCCn5nc(C(=O)NC(C)C)c(Br)c5C4)C3)c12 ; CCc1c(F)ccc2cc(O)cc(N3CCc4c(nc(OC[C@@]56CCCN5C[C@H](F)C6)nc4NCc4cc(C(=O)N(C)C)nn4C)C3)c12] 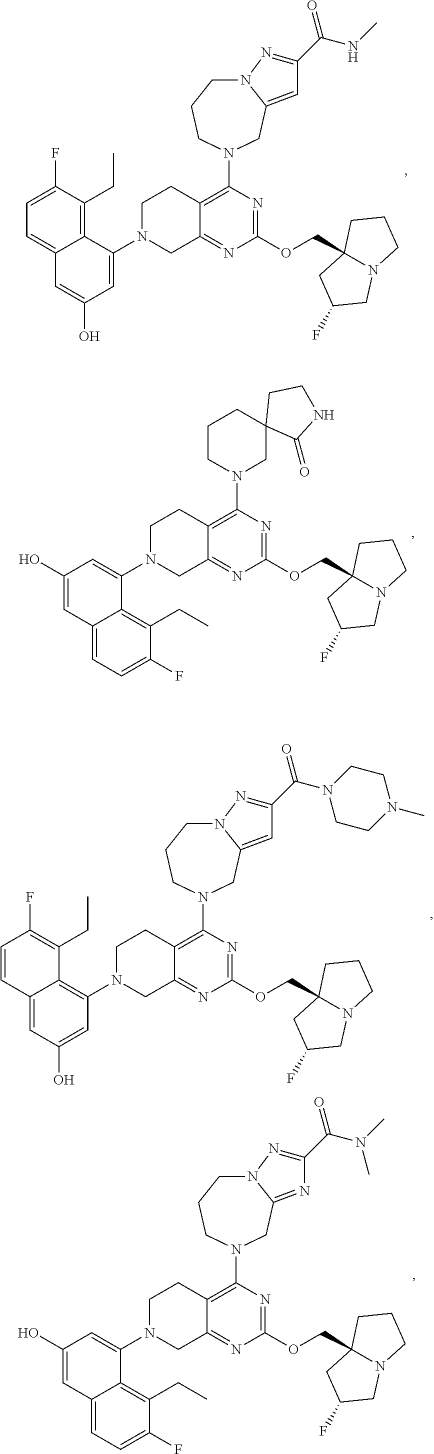 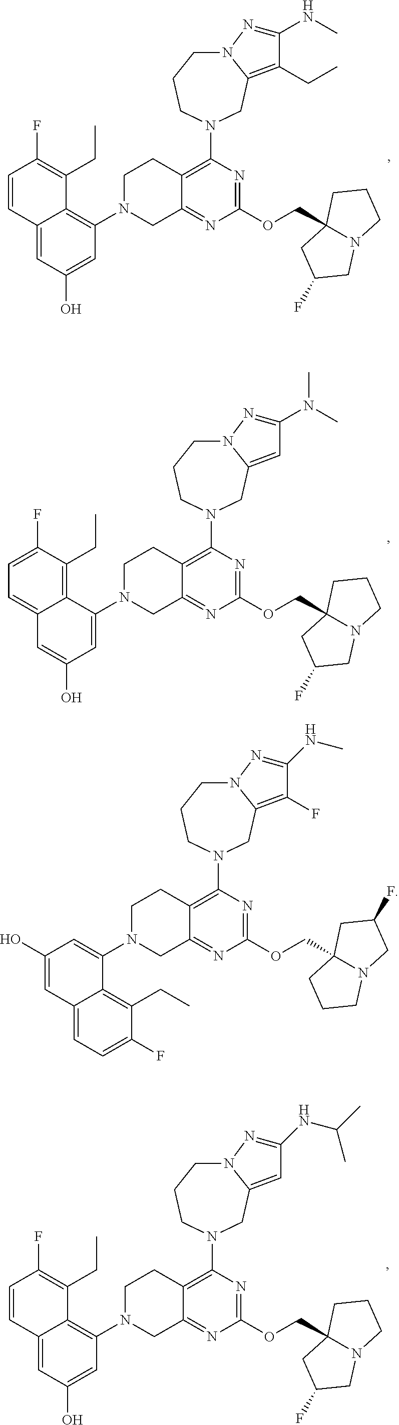 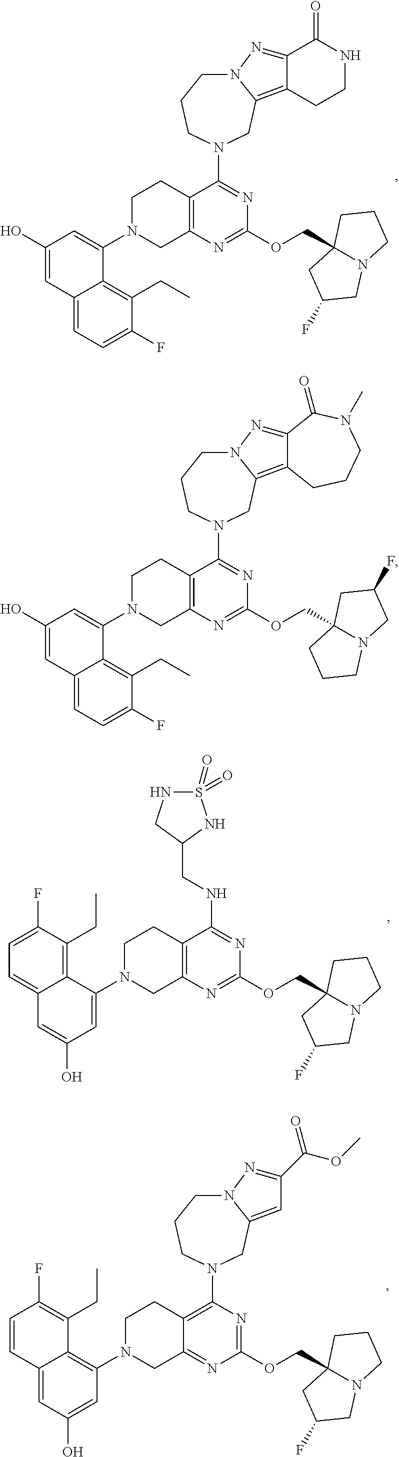 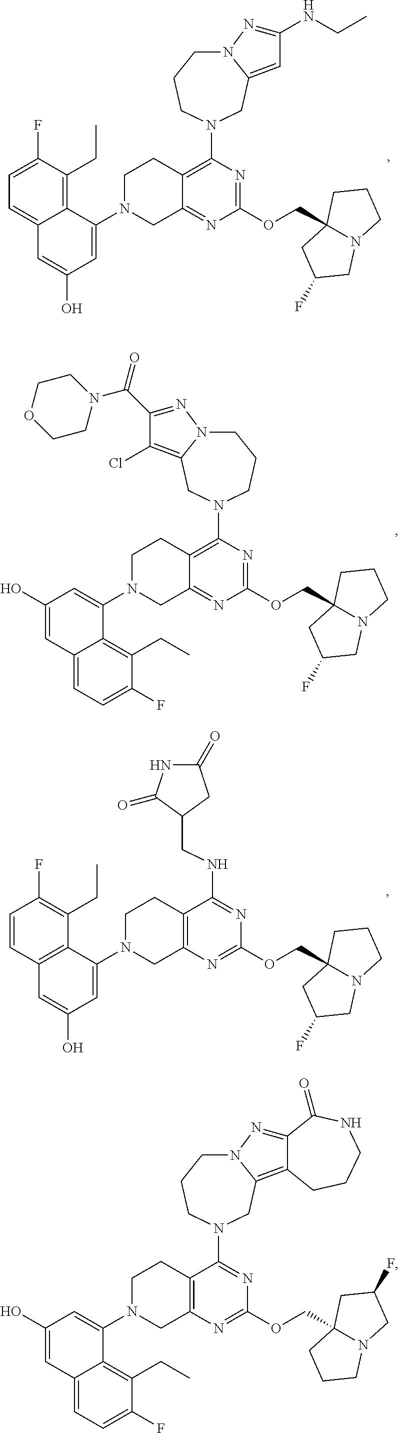 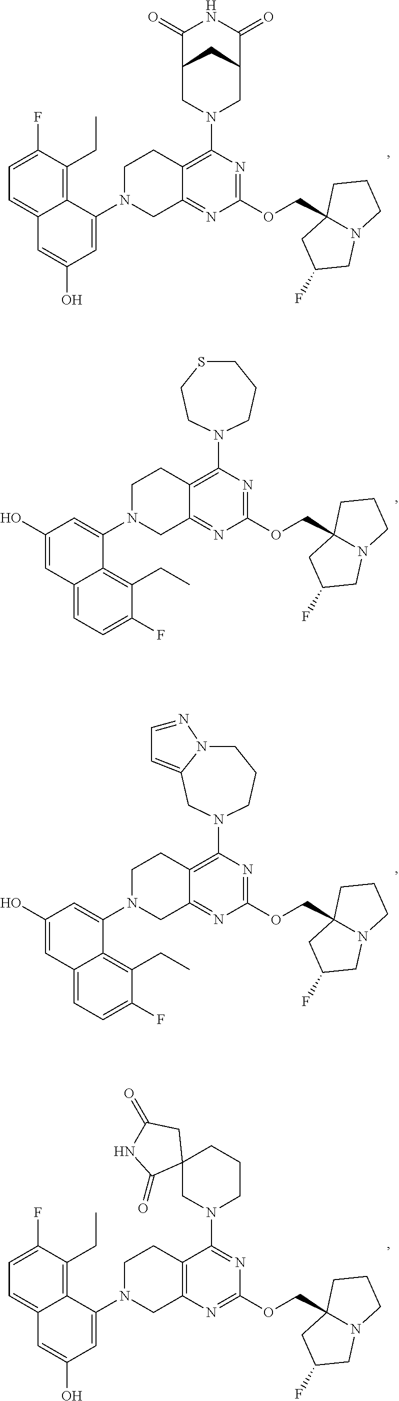 [SMILES: CCc1c(F)ccc2cc(O)cc(N3CCc4c(nc(OC[C@@]56CCCN5C[C@H](F)C6)nc4N4C[C@H]5C[C@@H](C4)C(=O)NC5=O)C3)c12 ; CCc1c(F)ccc2cc(O)cc(N3CCc4c(nc(OC[C@@]56CCCN5C[C@H](F)C6)nc4N4CCCSCC4)C3)c12 ; CCc1c(F)ccc2cc(O)cc(N3CCc4c(nc(OC[C@@]56CCCN5C[C@H](F)C6)nc4N4CCCn5nccc5C4)C3)c12 ; CCc1c(F)ccc2cc(O)cc(N3CCc4c(nc(OC[C@@]56CCCN5C[C@H](F)C6)nc4N4CCCC5(CC(=O)NC5=O)C4)C3)c12] 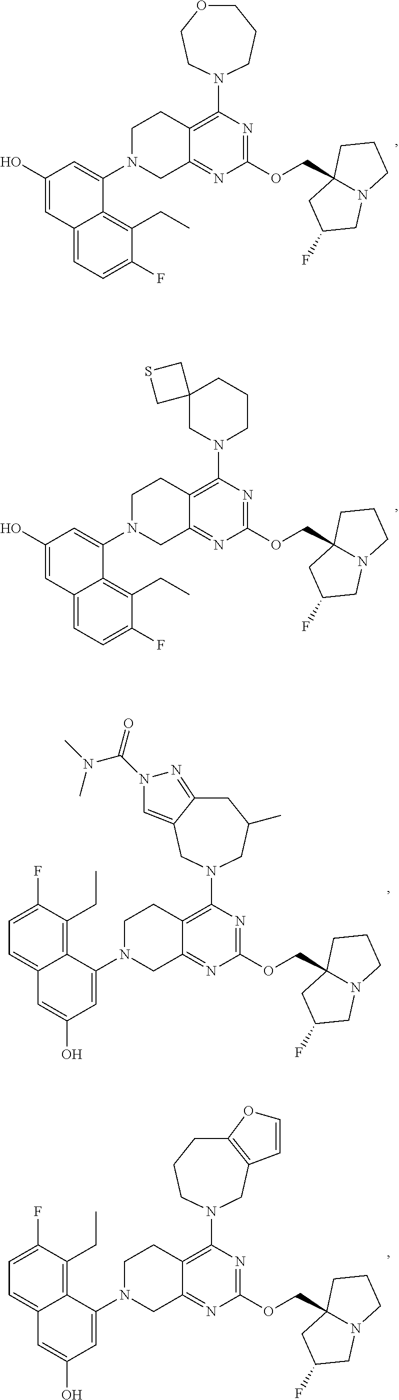 [SMILES: CCc1c(F)ccc2cc(O)cc(N3CCc4c(nc(OC[C@@]56CCCN5C[C@H](F)C6)nc4N4CCCOCC4)C3)c12 ; CCc1c(F)ccc2cc(O)cc(N3CCc4c(nc(OC[C@@]56CCCN5C[C@H](F)C6)nc4N4CCCC5(CSC5)C4)C3)c12 ; CCc1c(F)ccc2cc(O)cc(N3CCc4c(nc(OC[C@@]56CCCN5C[C@H](F)C6)nc4N4Cc5cn(C(=O)N(C)C)nc5CC(C)C4)C3)c12 ; CCc1c(F)ccc2cc(O)cc(N3CCc4c(nc(OC[C@@]56CCCN5C[C@H](F)C6)nc4N4CCCc5occc5C4)C3)c12] 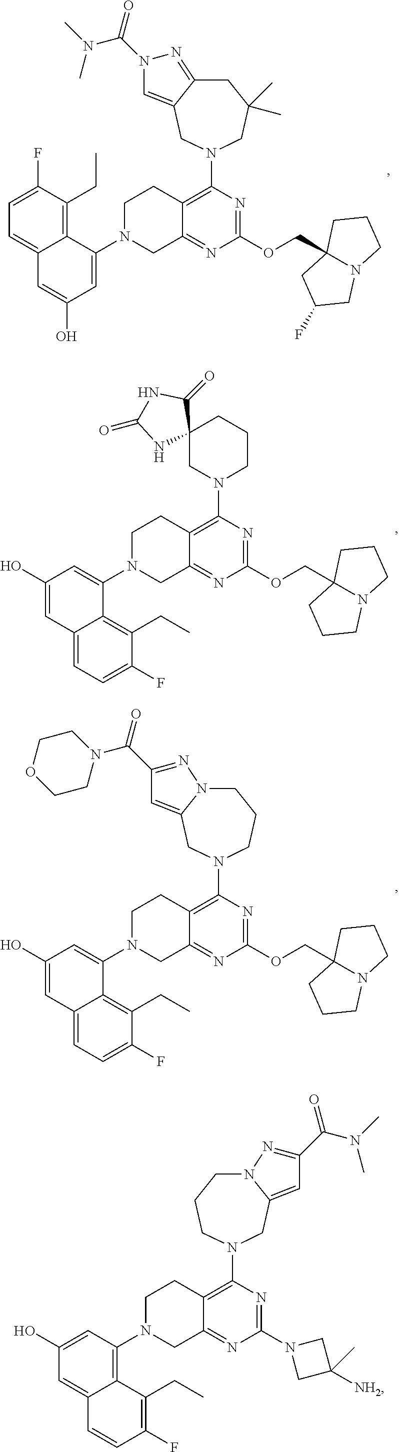 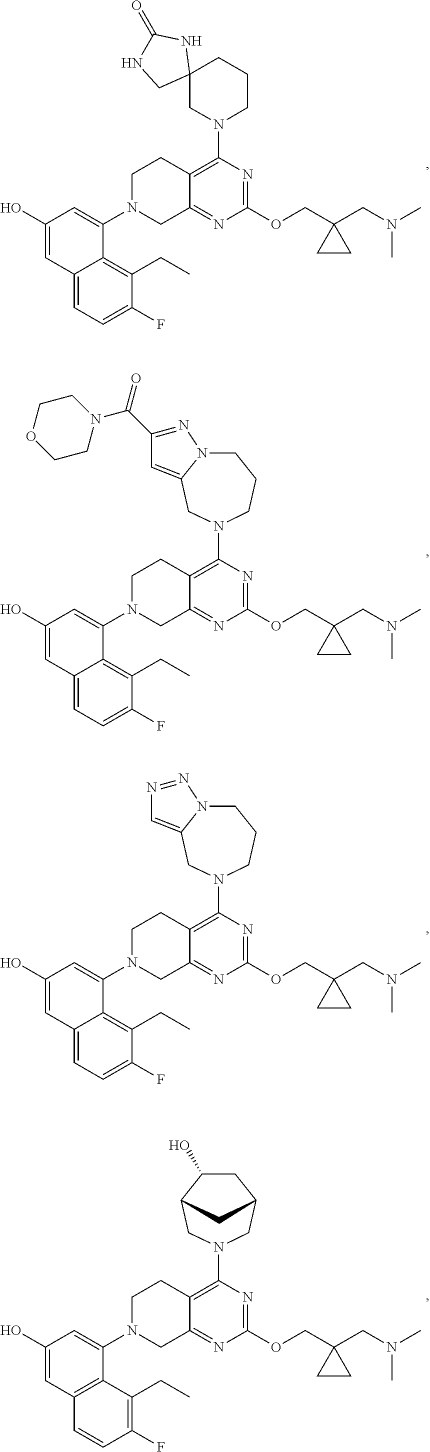 [SMILES: CCc1c(F)ccc2cc(O)cc(N3CCc4c(nc(OCC5(CN(C)C)CC5)nc4N4CCCC5(CNC(=O)N5)C4)C3)c12 ; CCc1c(F)ccc2cc(O)cc(N3CCc4c(nc(OCC5(CN(C)C)CC5)nc4N4CCCn5nc(C(=O)N6CCOCC6)cc5C4)C3)c12 ; CCc1c(F)ccc2cc(O)cc(N3CCc4c(nc(OCC5(CN(C)C)CC5)nc4N4CCCn5nncc5C4)C3)c12 ; CCc1c(F)ccc2cc(O)cc(N3CCc4c(nc(OCC5(CN(C)C)CC5)nc4N4C[C@@H]5C[C@H](C4)[C@H](O)C5)C3)c12] 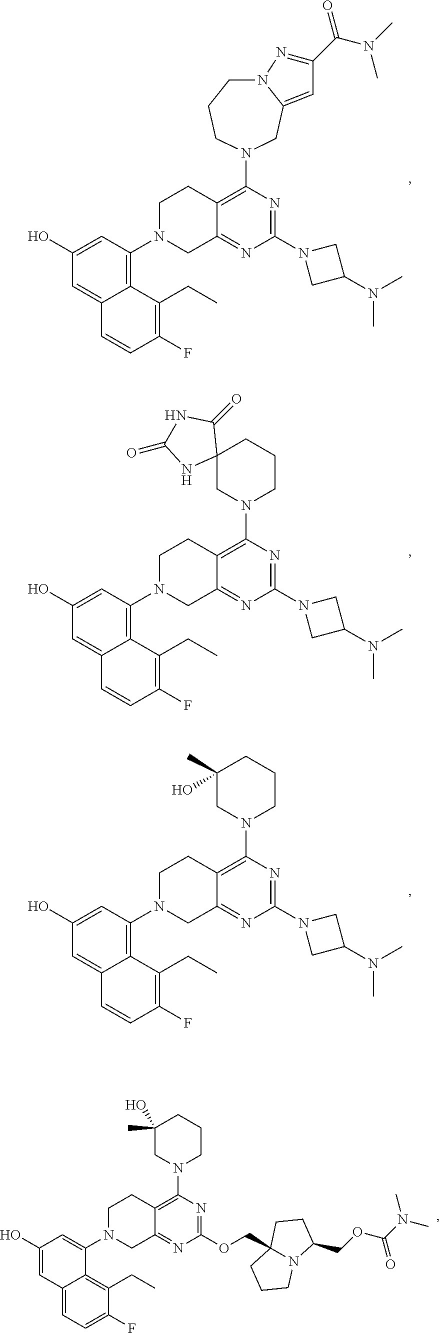 [SMILES: CCc1c(F)ccc2cc(O)cc(N3CCc4c(nc(N5CC(N(C)C)C5)nc4N4CCCn5nc(C(=O)N(C)C)cc5C4)C3)c12 ; CCc1c(F)ccc2cc(O)cc(N3CCc4c(nc(N5CC(N(C)C)C5)nc4N4CCCC5(C4)NC(=O)NC5=O)C3)c12 ; CCc1c(F)ccc2cc(O)cc(N3CCc4c(nc(N5CC(N(C)C)C5)nc4N4CCC[C@@](C)(O)C4)C3)c12 ; CCc1c(F)ccc2cc(O)cc(N3CCc4c(nc(OC[C@]56CCCN5[C@H](COC(=O)N(C)C)CC6)nc4N4CCC[C@@](C)(O)C4)C3)c12] 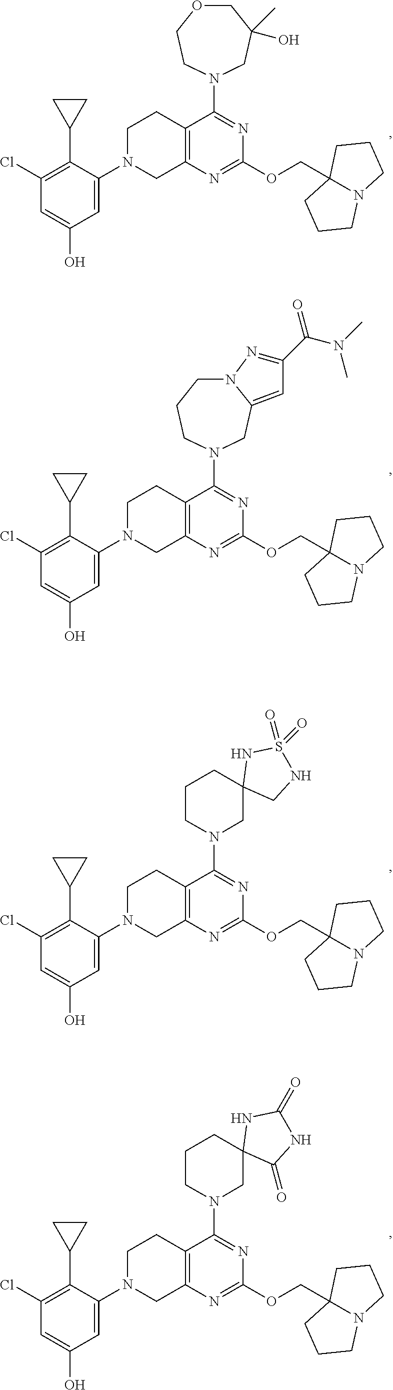 [SMILES: CC1(O)COCCN(c2nc(OCC34CCCN3CCC4)nc3c2CCN(c2cc(O)cc(Cl)c2C2CC2)C3)C1 ; CN(C)C(=O)c1cc2n(n1)CCCN(c1nc(OCC34CCCN3CCC4)nc3c1CCN(c1cc(O)cc(Cl)c1C1CC1)C3)C2 ; O=S1(=O)NCC2(CCCN(c3nc(OCC45CCCN4CCC5)nc4c3CCN(c3cc(O)cc(Cl)c3C3CC3)C4)C2)N1 ; O=C1NC(=O)C2(CCCN(c3nc(OCC45CCCN4CCC5)nc4c3CCN(c3cc(O)cc(Cl)c3C3CC3)C4)C2)N1] 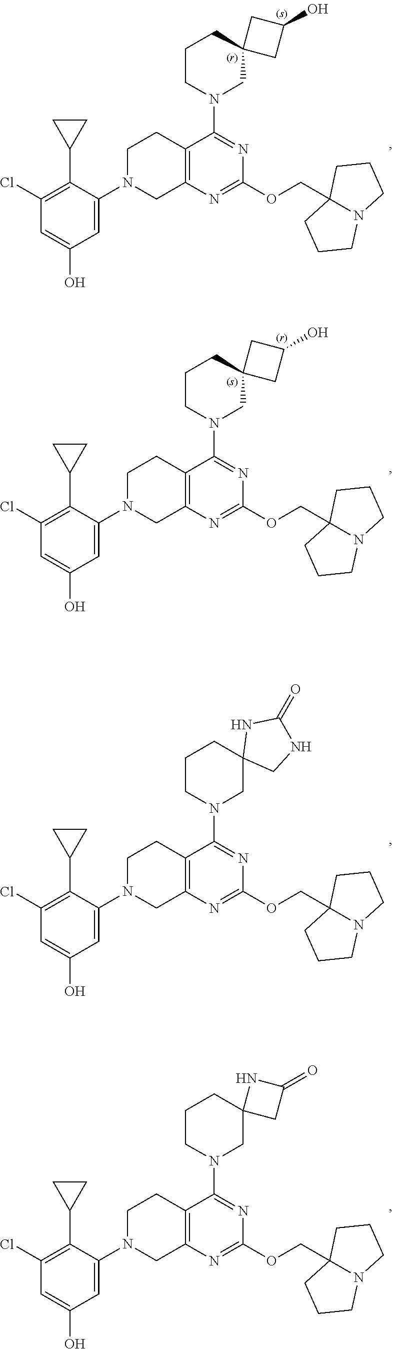 [SMILES: Oc1cc(Cl)c(C2CC2)c(N2CCc3c(nc(OCC45CCCN4CCC5)nc3N3CCC[C@]4(C3)C[C@H](O)C4)C2)c1 ; Oc1cc(Cl)c(C2CC2)c(N2CCc3c(nc(OCC45CCCN4CCC5)nc3N3CCC[C@]4(C3)C[C@@H](O)C4)C2)c1 ; O=C1NCC2(CCCN(c3nc(OCC45CCCN4CCC5)nc4c3CCN(c3cc(O)cc(Cl)c3C3CC3)C4)C2)N1 ; O=C1CC2(CCCN(c3nc(OCC45CCCN4CCC5)nc4c3CCN(c3cc(O)cc(Cl)c3C3CC3)C4)C2)N1] 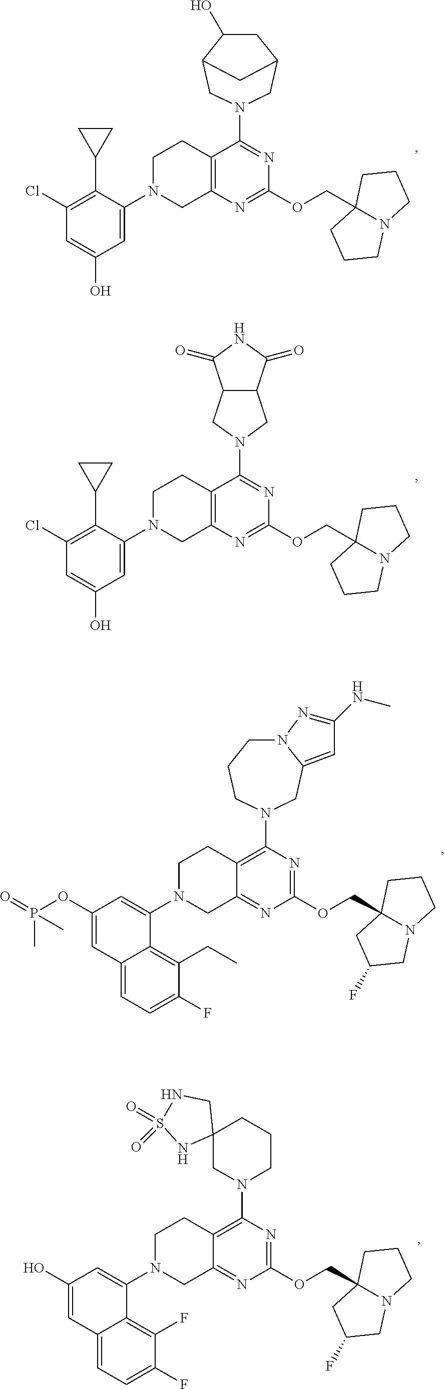 [SMILES: Oc1cc(Cl)c(C2CC2)c(N2CCc3c(nc(OCC45CCCN4CCC5)nc3N3CC4CC(O)C(C4)C3)C2)c1 ; O=C1NC(=O)C2CN(c3nc(OCC45CCCN4CCC5)nc4c3CCN(c3cc(O)cc(Cl)c3C3CC3)C4)CC12 ; CCc1c(F)ccc2cc(OP(C)(C)=O)cc(N3CCc4c(nc(OC[C@@]56CCCN5C[C@H](F)C6)nc4N4CCCn5nc(NC)cc5C4)C3)c12 ; O=S1(=O)NCC2(CCCN(c3nc(OC[C@@]45CCCN4C[C@H](F)C5)nc4c3CCN(c3cc(O)cc5ccc(F)c(F)c35)C4)C2)N1] 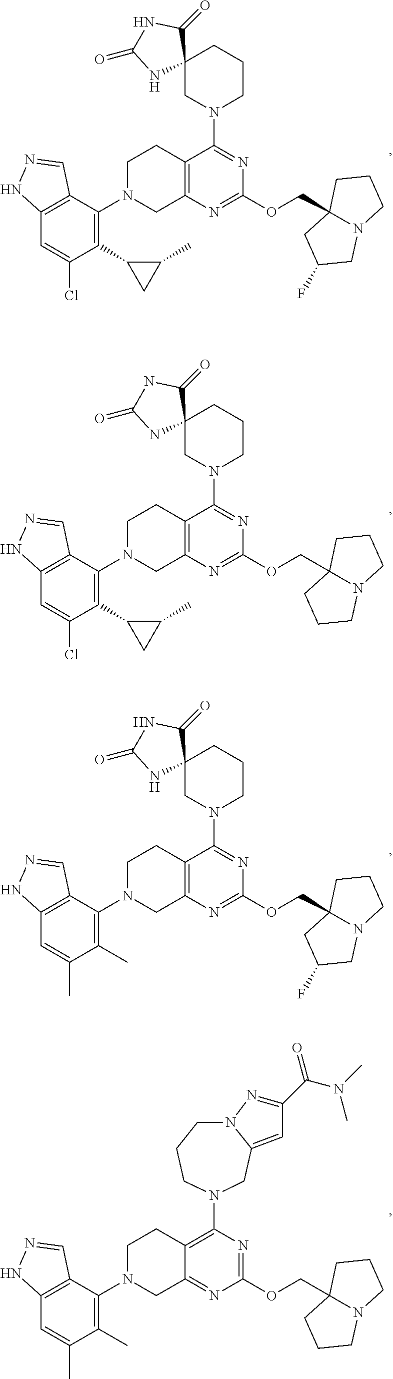 [SMILES: C[C@@H]1C[C@@H]1c1c(Cl)cc2[nH]ncc2c1N1CCc2c(nc(OC[C@@]34CCCN3C[C@H](F)C4)nc2N2CCC[C@]3(C2)NC(=O)NC3=O)C1 ; C[C@@H]1C[C@@H]1c1c(Cl)cc2[nH]ncc2c1N1CCc2c(nc(OCC34CCCN3CCC4)nc2N2CCC[C@]3(C2)NC(=O)NC3=O)C1 ; Cc1cc2[nH]ncc2c(N2CCc3c(nc(OC[C@@]45CCCN4C[C@H](F)C5)nc3N3CCC[C@]4(C3)NC(=O)NC4=O)C2)c1C ; Cc1cc2[nH]ncc2c(N2CCc3c(nc(OCC45CCCN4CCC5)nc3N3CCCn4nc(C(=O)N(C)C)cc4C3)C2)c1C] 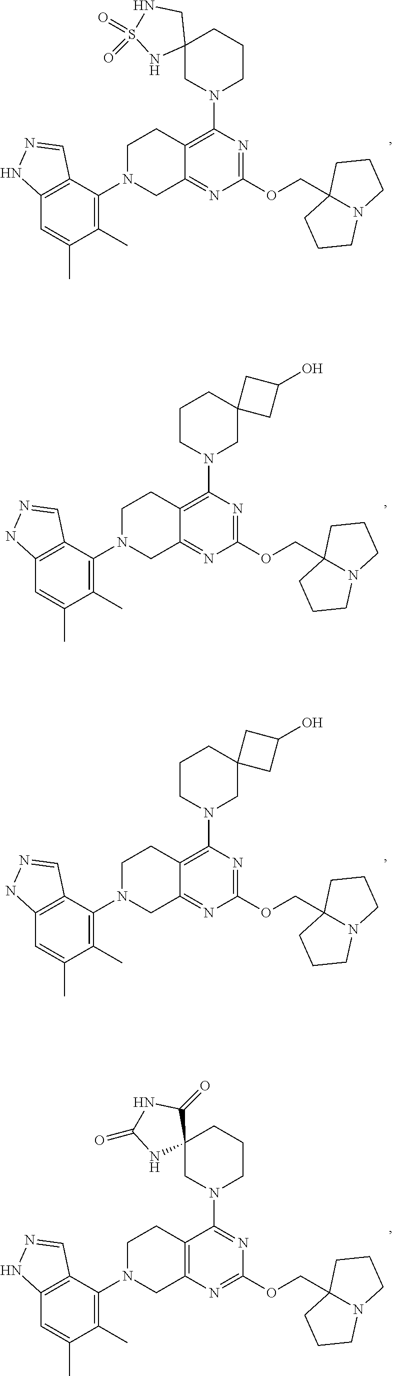 [SMILES: Cc1cc2[nH]ncc2c(N2CCc3c(nc(OCC45CCCN4CCC5)nc3N3CCCC4(CNS(=O)(=O)N4)C3)C2)c1C ; Cc1cc2[nH]ncc2c(N2CCc3c(nc(OCC45CCCN4CCC5)nc3N3CCCC4(CC(O)C4)C3)C2)c1C ; Cc1cc2[nH]ncc2c(N2CCc3c(nc(OCC45CCCN4CCC5)nc3N3CCCC4(CC(O)C4)C3)C2)c1C ; Cc1cc2[nH]ncc2c(N2CCc3c(nc(OCC45CCCN4CCC5)nc3N3CCC[C@]4(C3)NC(=O)NC4=O)C2)c1C] 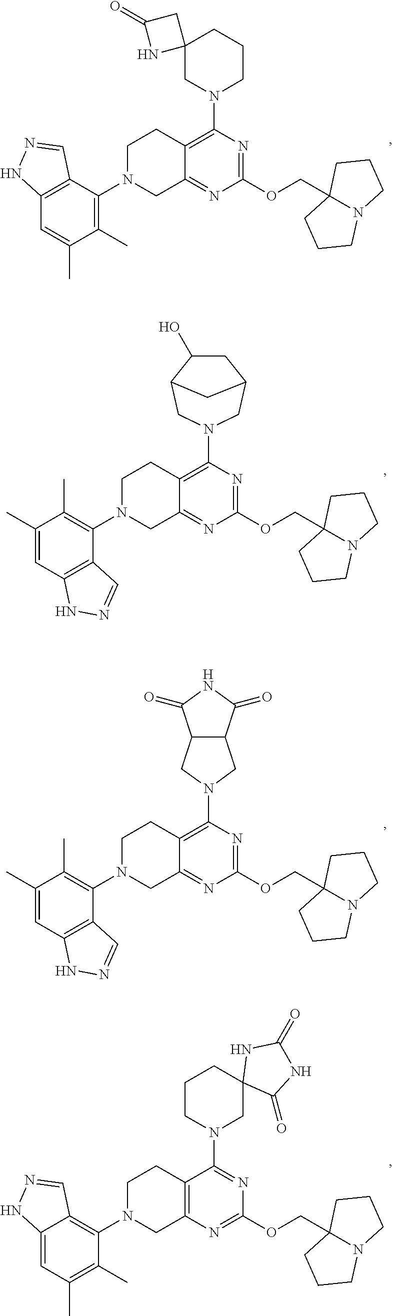 [SMILES: Cc1cc2[nH]ncc2c(N2CCc3c(nc(OCC45CCCN4CCC5)nc3N3CCCC4(CC(=O)N4)C3)C2)c1C ; Cc1cc2[nH]ncc2c(N2CCc3c(nc(OCC45CCCN4CCC5)nc3N3CC4CC(O)C(C4)C3)C2)c1C ; Cc1cc2[nH]ncc2c(N2CCc3c(nc(OCC45CCCN4CCC5)nc3N3CC4C(=O)NC(=O)C4C3)C2)c1C ; Cc1cc2[nH]ncc2c(N2CCc3c(nc(OCC45CCCN4CCC5)nc3N3CCCC4(C3)NC(=O)NC4=O)C2)c1C] 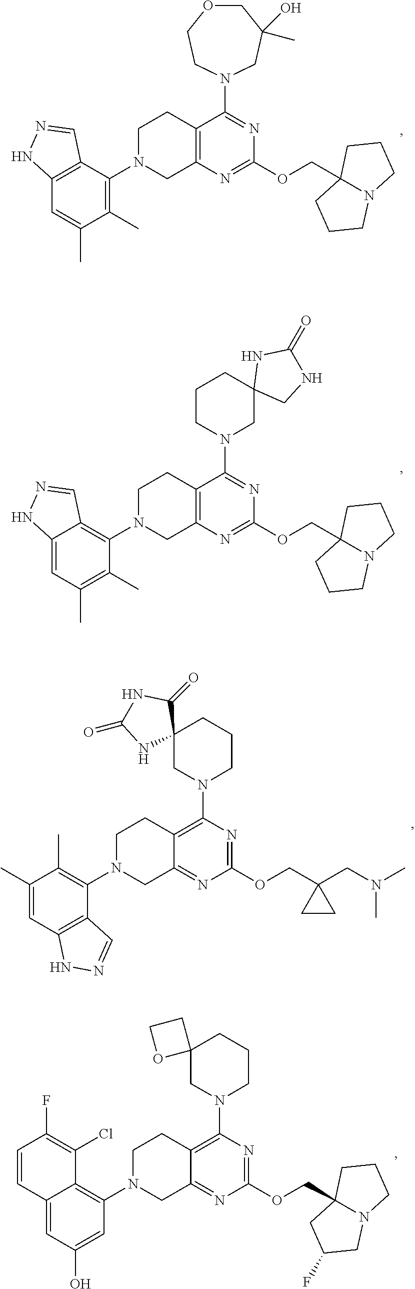 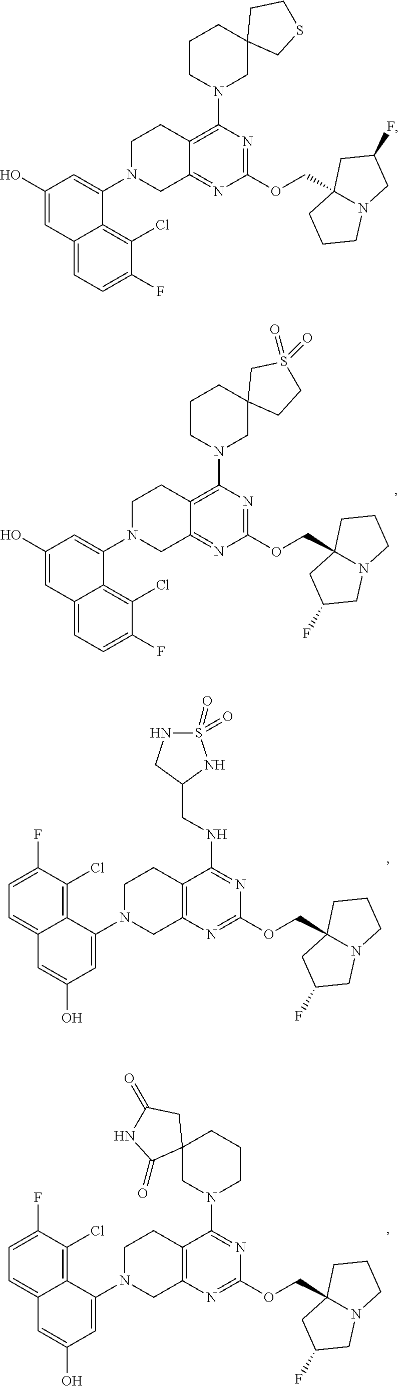 [SMILES: Oc1cc(N2CCc3c(nc(OC[C@@]45CCCN4C[C@H](F)C5)nc3N3CCCC4(CCSC4)C3)C2)c2c(Cl)c(F)ccc2c1 ; O=S1(=O)CCC2(CCCN(c3nc(OC[C@@]45CCCN4C[C@H](F)C5)nc4c3CCN(c3cc(O)cc5ccc(F)c(Cl)c35)C4)C2)C1 ; O=S1(=O)NCC(CNc2nc(OC[C@@]34CCCN3C[C@H](F)C4)nc3c2CCN(c2cc(O)cc4ccc(F)c(Cl)c24)C3)N1 ; O=C1CC2(CCCN(c3nc(OC[C@@]45CCCN4C[C@H](F)C5)nc4c3CCN(c3cc(O)cc5ccc(F)c(Cl)c35)C4)C2)C(=O)N1] 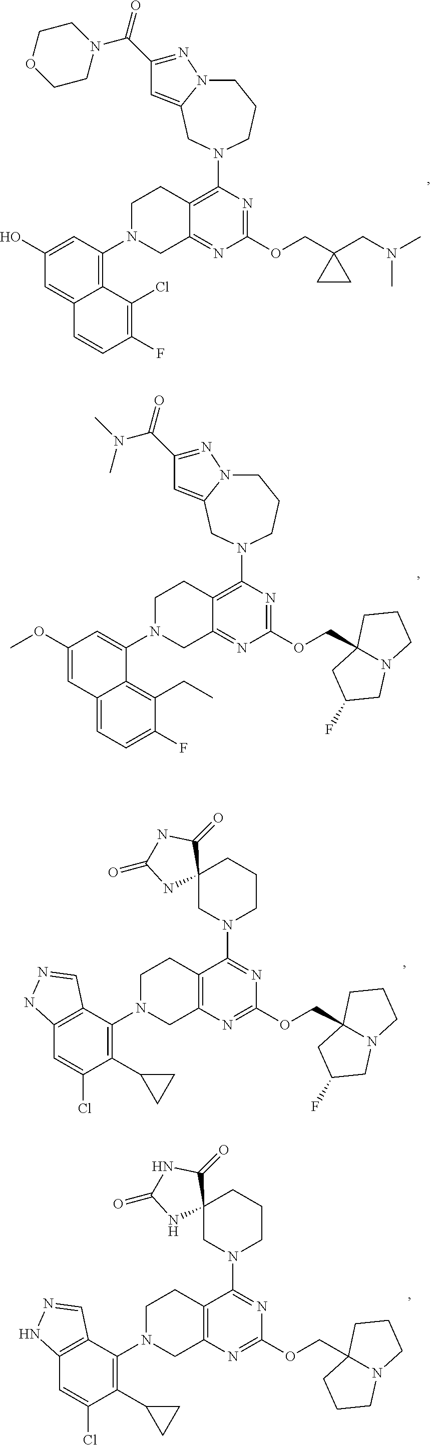 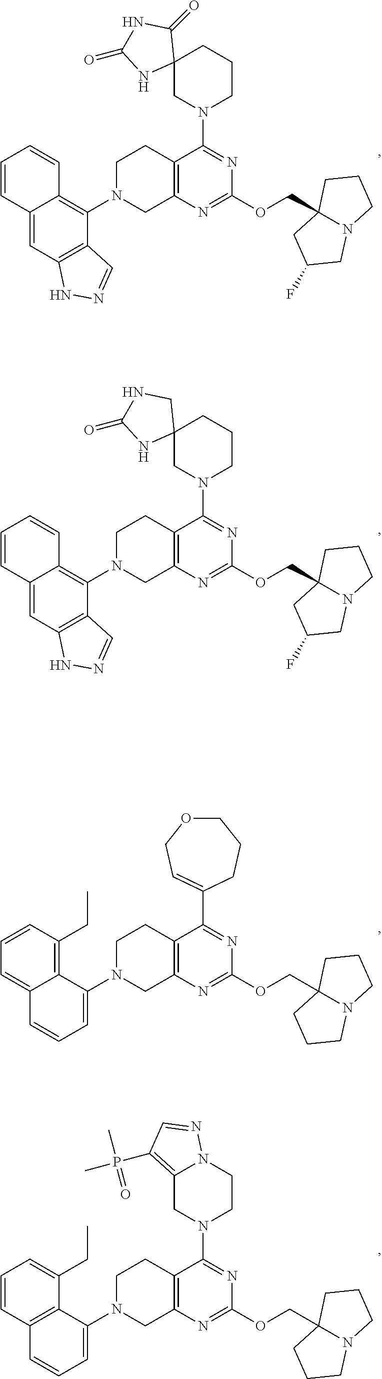 [SMILES: O=C1NC(=O)C2(CCCN(c3nc(OC[C@@]45CCCN4C[C@H](F)C5)nc4c3CCN(c3c5ccccc5cc5[nH]ncc35)C4)C2)N1 ; O=C1NCC2(CCCN(c3nc(OC[C@@]45CCCN4C[C@H](F)C5)nc4c3CCN(c3c5ccccc5cc5[nH]ncc35)C4)C2)N1 ; CCc1cccc2cccc(N3CCc4c(nc(OCC56CCCN5CCC6)nc4C4=CCOCCC4)C3)c12 ; CCc1cccc2cccc(N3CCc4c(nc(OCC56CCCN5CCC6)nc4N4CCn5ncc(P(C)(C)=O)c5C4)C3)c12] 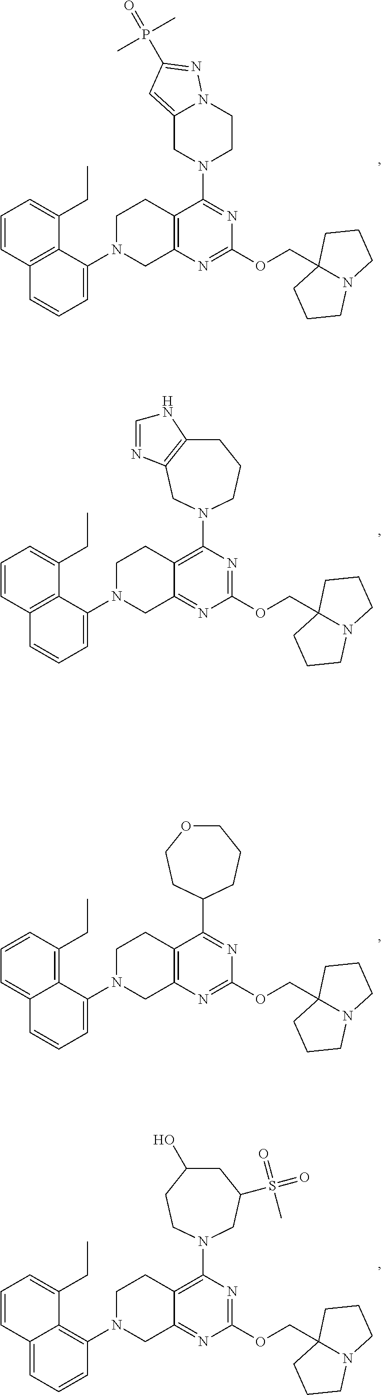 [SMILES: CCc1cccc2cccc(N3CCc4c(nc(OCC56CCCN5CCC6)nc4N4CCn5nc(P(C)(C)=O)cc5C4)C3)c12 ; CCc1cccc2cccc(N3CCc4c(nc(OCC56CCCN5CCC6)nc4N4CCCc5[nH]cnc5C4)C3)c12 ; CCc1cccc2cccc(N3CCc4c(nc(OCC56CCCN5CCC6)nc4C4CCCOCC4)C3)c12 ; CCc1cccc2cccc(N3CCc4c(nc(OCC56CCCN5CCC6)nc4N4CCC(O)CC(S(C)(=O)=O)C4)C3)c12] 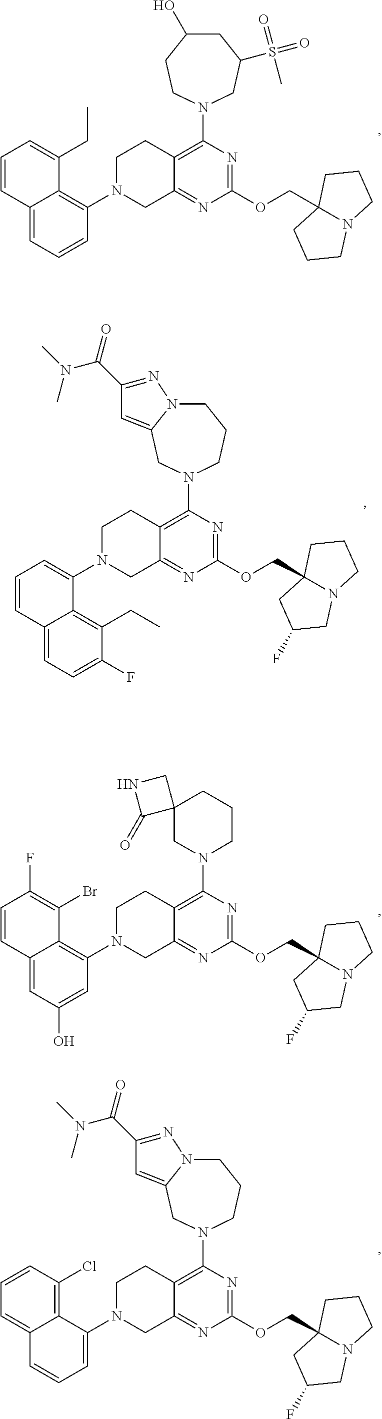 [SMILES: CCc1cccc2cccc(N3CCc4c(nc(OCC56CCCN5CCC6)nc4N4CCC(O)CC(S(C)(=O)=O)C4)C3)c12 ; CCc1c(F)ccc2cccc(N3CCc4c(nc(OC[C@@]56CCCN5C[C@H](F)C6)nc4N4CCCn5nc(C(=O)N(C)C)cc5C4)C3)c12 ; O=C1NCC12CCCN(c1nc(OC[C@@]34CCCN3C[C@H](F)C4)nc3c1CCN(c1cc(O)cc4ccc(F)c(Br)c14)C3)C2 ; CN(C)C(=O)c1cc2n(n1)CCCN(c1nc(OC[C@@]34CCCN3C[C@H](F)C4)nc3c1CCN(c1cccc4cccc(Cl)c14)C3)C2] 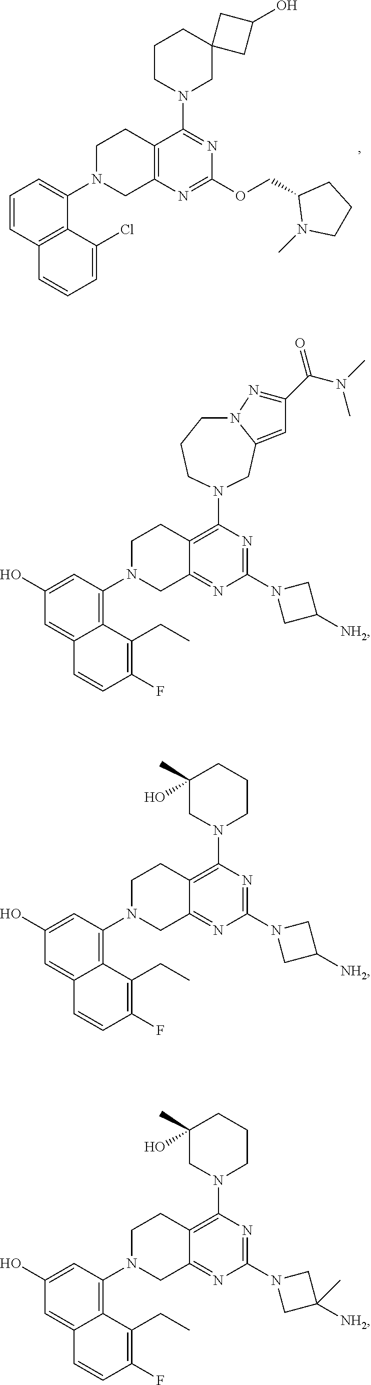 [SMILES: CN1CCC[C@H]1COc1nc2c(c(N3CCCC4(CC(O)C4)C3)n1)CCN(c1cccc3cccc(Cl)c13)C2 ; CCc1c(F)ccc2cc(O)cc(N3CCc4c(nc(N5CC(N)C5)nc4N4CCCn5nc(C(=O)N(C)C)cc5C4)C3)c12 ; CCc1c(F)ccc2cc(O)cc(N3CCc4c(nc(N5CC(N)C5)nc4N4CCC[C@@](C)(O)C4)C3)c12 ; CCc1c(F)ccc2cc(O)cc(N3CCc4c(nc(N5CC(C)(N)C5)nc4N4CCC[C@@](C)(O)C4)C3)c12] 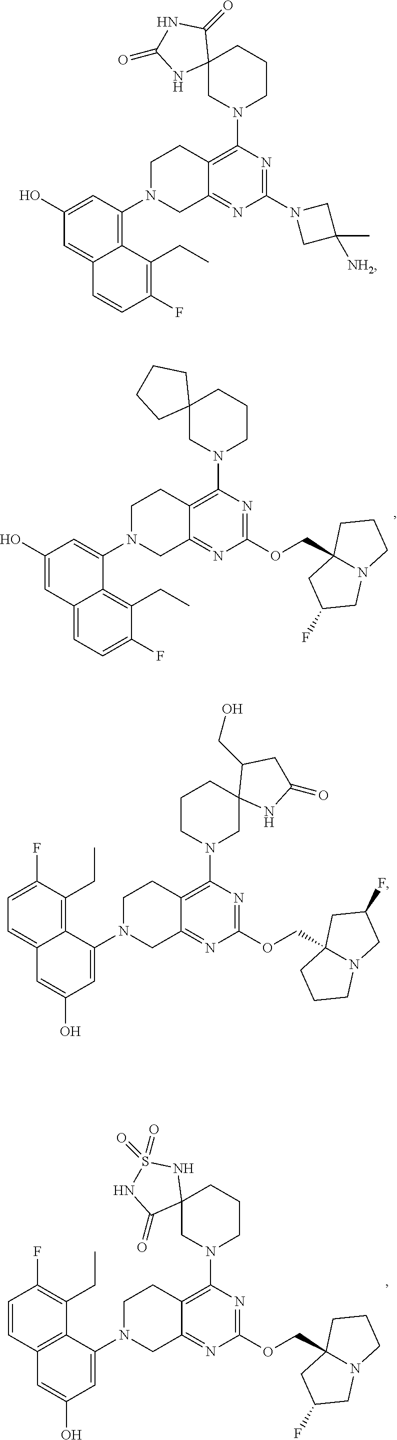 [SMILES: CCc1c(F)ccc2cc(O)cc(N3CCc4c(nc(N5CC(C)(N)C5)nc4N4CCCC5(C4)NC(=O)NC5=O)C3)c12 ; CCc1c(F)ccc2cc(O)cc(N3CCc4c(nc(OC[C@@]56CCCN5C[C@H](F)C6)nc4N4CCCC5(CCCC5)C4)C3)c12 ; CCc1c(F)ccc2cc(O)cc(N3CCc4c(nc(OC[C@@]56CCCN5C[C@H](F)C6)nc4N4CCCC5(C4)NC(=O)CC5CO)C3)c12 ; CCc1c(F)ccc2cc(O)cc(N3CCc4c(nc(OC[C@@]56CCCN5C[C@H](F)C6)nc4N4CCCC5(C4)NS(=O)(=O)NC5=O)C3)c12] 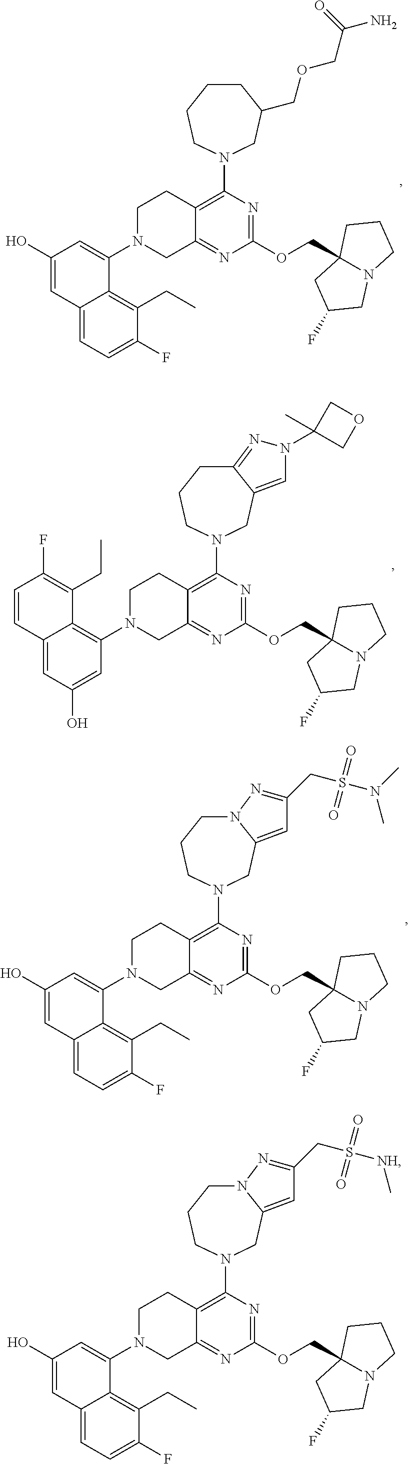 [SMILES: CCc1c(F)ccc2cc(O)cc(N3CCc4c(nc(OC[C@@]56CCCN5C[C@H](F)C6)nc4N4CCCCC(COCC(N)=O)C4)C3)c12 ; CCc1c(F)ccc2cc(O)cc(N3CCc4c(nc(OC[C@@]56CCCN5C[C@H](F)C6)nc4N4CCCc5nn(C6(C)COC6)cc5C4)C3)c12 ; CCc1c(F)ccc2cc(O)cc(N3CCc4c(nc(OC[C@@]56CCCN5C[C@H](F)C6)nc4N4CCCn5nc(CS(=O)(=O)N(C)C)cc5C4)C3)c12 ; CCc1c(F)ccc2cc(O)cc(N3CCc4c(nc(OC[C@@]56CCCN5C[C@H](F)C6)nc4N4CCCn5nc(CS(=O)(=O)NC)cc5C4)C3)c12] 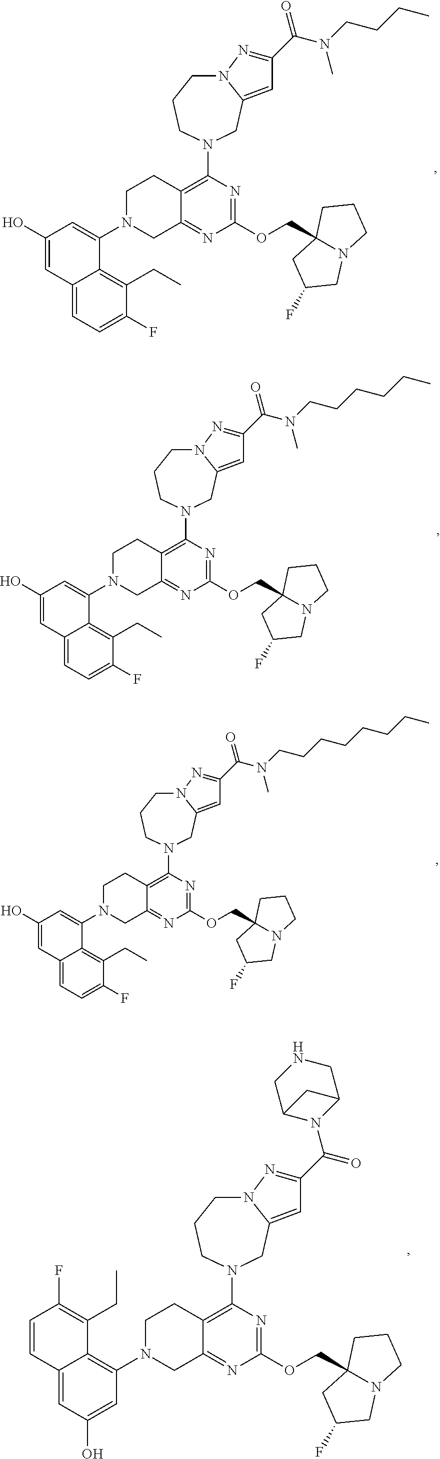 [SMILES: CCCCN(C)C(=O)c1cc2n(n1)CCCN(c1nc(OC[C@@]34CCCN3C[C@H](F)C4)nc3c1CCN(c1cc(O)cc4ccc(F)c(CC)c14)C3)C2 ; CCCCCCN(C)C(=O)c1cc2n(n1)CCCN(c1nc(OC[C@@]34CCCN3C[C@H](F)C4)nc3c1CCN(c1cc(O)cc4ccc(F)c(CC)c14)C3)C2 ; CCCCCCCCN(C)C(=O)c1cc2n(n1)CCCN(c1nc(OC[C@@]34CCCN3C[C@H](F)C4)nc3c1CCN(c1cc(O)cc4ccc(F)c(CC)c14)C3)C2 ; CCc1c(F)ccc2cc(O)cc(N3CCc4c(nc(OC[C@@]56CCCN5C[C@H](F)C6)nc4N4CCCn5nc(C(=O)N6C7CNCC6C7)cc5C4)C3)c12] 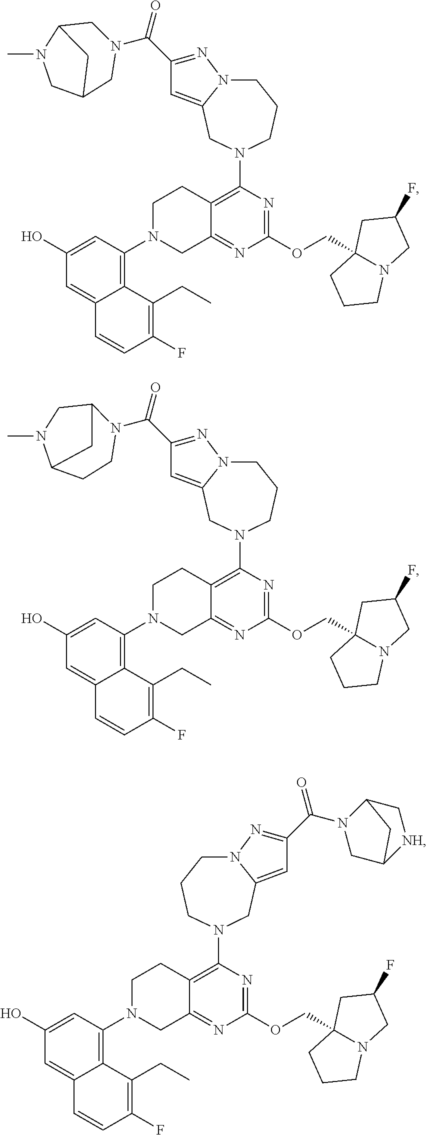 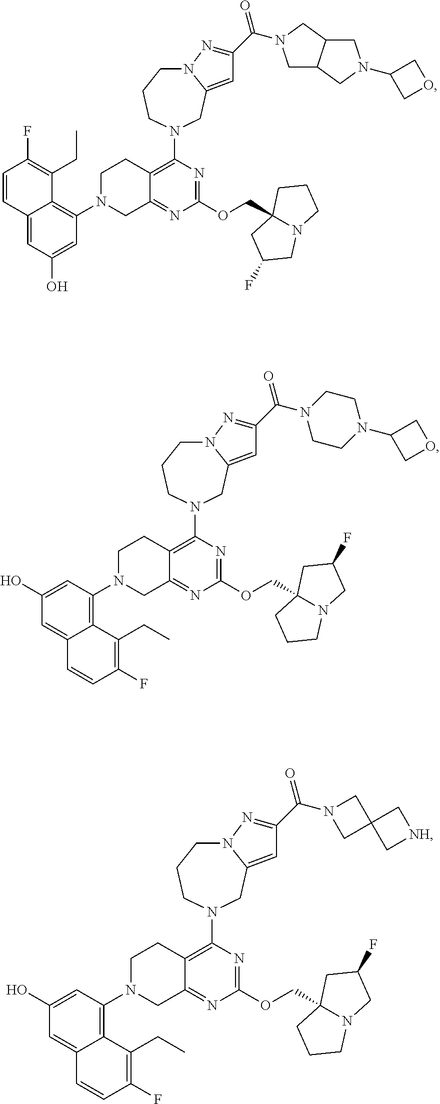 [SMILES: CCc1c(F)ccc2cc(O)cc(N3CCc4c(nc(OC[C@@]56CCCN5C[C@H](F)C6)nc4N4CCCn5nc(C(=O)N6CC7CN(C8COC8)CC7C6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CCN(C7COC7)CC6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC7(CNC7)C6)cc5C4)C3)c12] 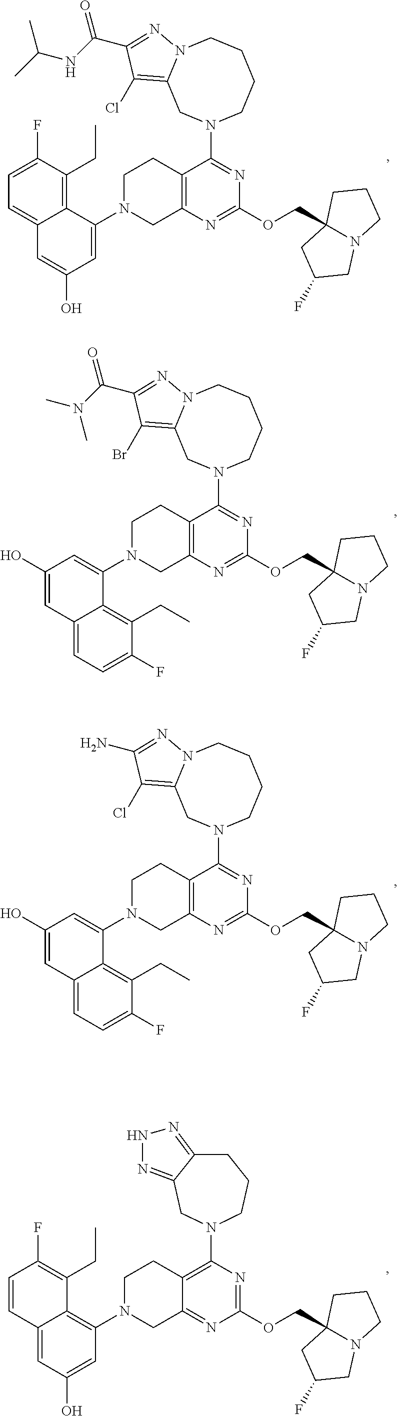 [SMILES: CCc1c(F)ccc2cc(O)cc(N3CCc4c(nc(OC[C@@]56CCCN5C[C@H](F)C6)nc4N4CCCCn5nc(C(=O)NC(C)C)c(Cl)c5C4)C3)c12 ; CCc1c(F)ccc2cc(O)cc(N3CCc4c(nc(OC[C@@]56CCCN5C[C@H](F)C6)nc4N4CCCCn5nc(C(=O)N(C)C)c(Br)c5C4)C3)c12 ; CCc1c(F)ccc2cc(O)cc(N3CCc4c(nc(OC[C@@]56CCCN5C[C@H](F)C6)nc4N4CCCCn5nc(N)c(Cl)c5C4)C3)c12 ; CCc1c(F)ccc2cc(O)cc(N3CCc4c(nc(OC[C@@]56CCCN5C[C@H](F)C6)nc4N4CCCc5n[nH]nc5C4)C3)c12] 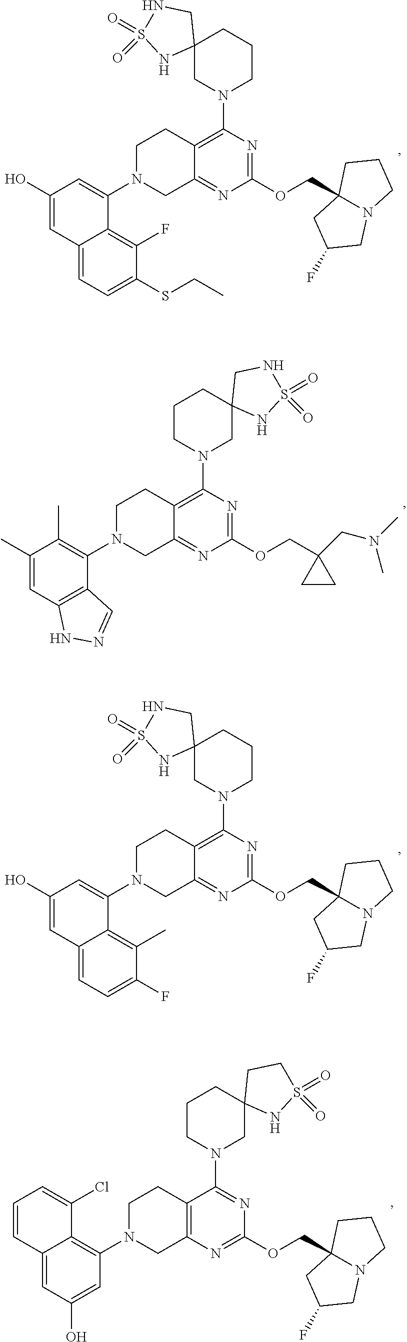 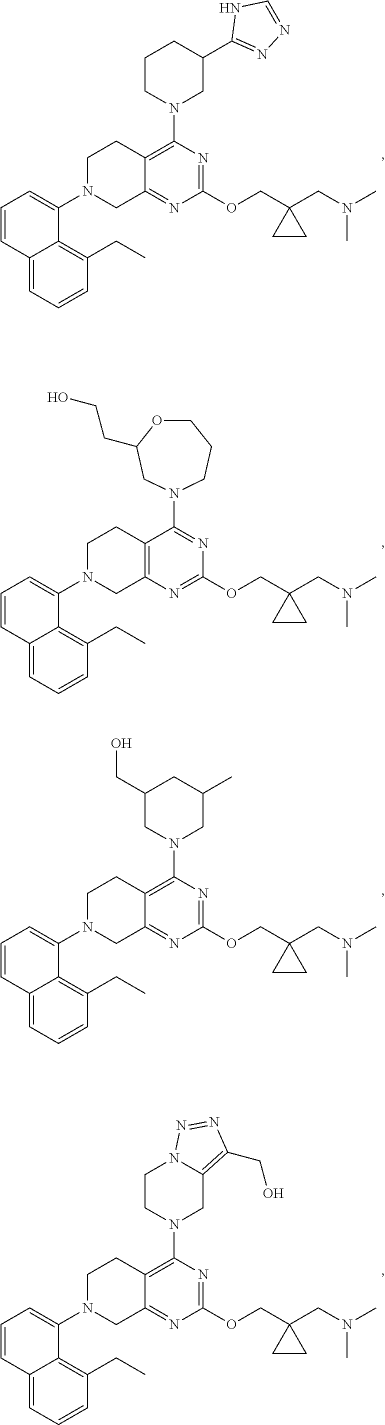 [SMILES: CCc1cccc2cccc(N3CCc4c(nc(OCC5(CN(C)C)CC5)nc4N4CCCC(c5nnc[nH]5)C4)C3)c12 ; CCc1cccc2cccc(N3CCc4c(nc(OCC5(CN(C)C)CC5)nc4N4CCCOC(CCO)C4)C3)c12 ; CCc1cccc2cccc(N3CCc4c(nc(OCC5(CN(C)C)CC5)nc4N4CC(C)CC(CO)C4)C3)c12 ; CCc1cccc2cccc(N3CCc4c(nc(OCC5(CN(C)C)CC5)nc4N4CCn5nnc(CO)c5C4)C3)c12] 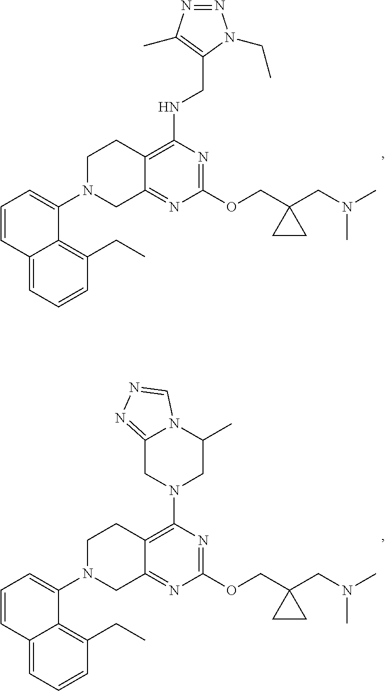 [SMILES: CCc1cccc2cccc(N3CCc4c(nc(OCC5(CN(C)C)CC5)nc4NCc4c(C)nnn4CC)C3)c12 ; CCc1cccc2cccc(N3CCc4c(nc(OCC5(CN(C)C)CC5)nc4N4Cc5nncn5C(C)C4)C3)c12] 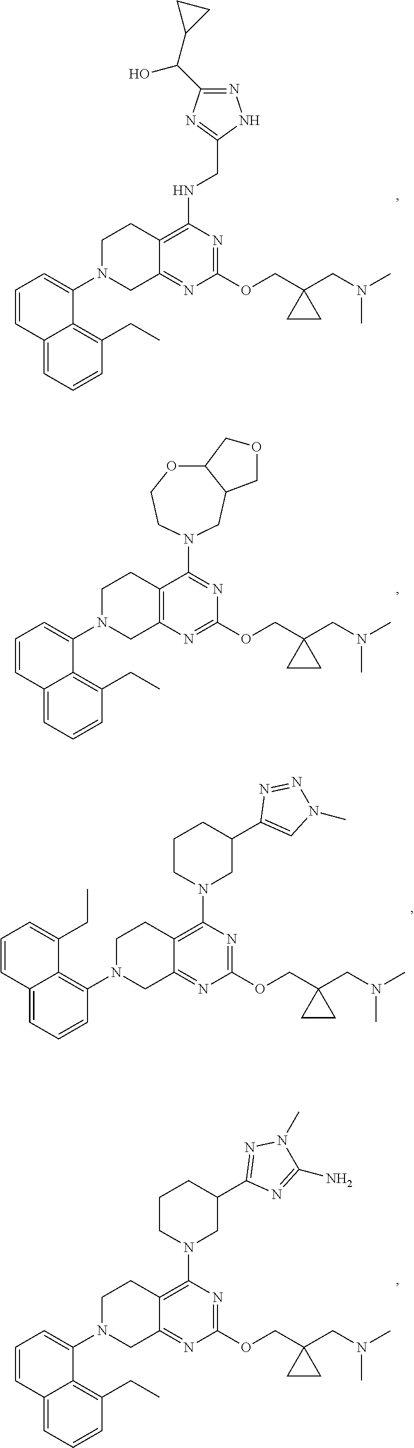 [SMILES: CCc1cccc2cccc(N3CCc4c(nc(OCC5(CN(C)C)CC5)nc4NCc4nc(C(O)C5CC5)n[nH]4)C3)c12 ; CCc1cccc2cccc(N3CCc4c(nc(OCC5(CN(C)C)CC5)nc4N4CCOC5COCC5C4)C3)c12 ; CCc1cccc2cccc(N3CCc4c(nc(OCC5(CN(C)C)CC5)nc4N4CCCC(c5cn(C)nn5)C4)C3)c12 ; CCc1cccc2cccc(N3CCc4c(nc(OCC5(CN(C)C)CC5)nc4N4CCCC(c5nc(N)n(C)n5)C4)C3)c12] 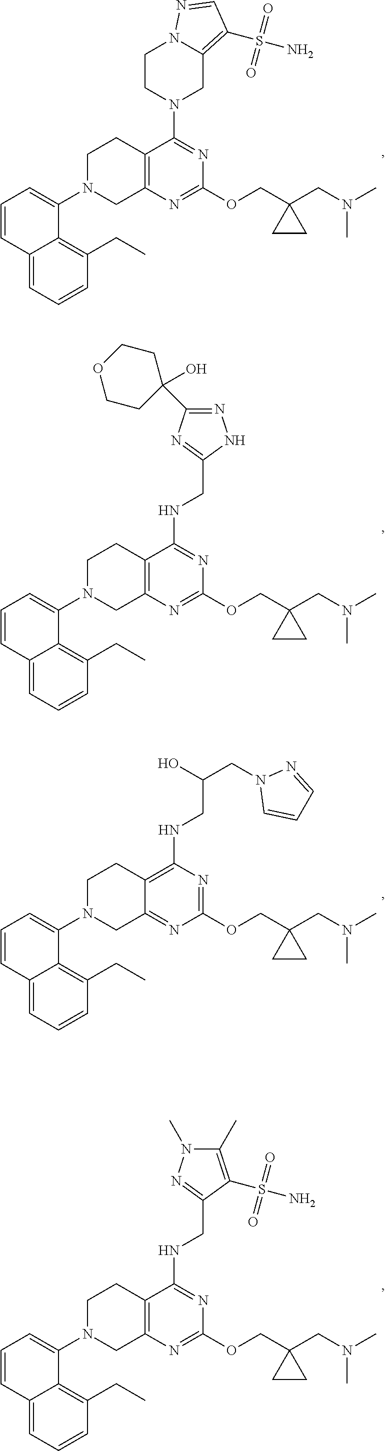 [SMILES: CCc1cccc2cccc(N3CCc4c(nc(OCC5(CN(C)C)CC5)nc4N4CCn5ncc(S(N)(=O)=O)c5C4)C3)c12 ; CCc1cccc2cccc(N3CCc4c(nc(OCC5(CN(C)C)CC5)nc4NCc4nc(C5(O)CCOCC5)n[nH]4)C3)c12 ; CCc1cccc2cccc(N3CCc4c(nc(OCC5(CN(C)C)CC5)nc4NCC(O)Cn4cccn4)C3)c12 ; CCc1cccc2cccc(N3CCc4c(nc(OCC5(CN(C)C)CC5)nc4NCc4nn(C)c(C)c4S(N)(=O)=O)C3)c12] 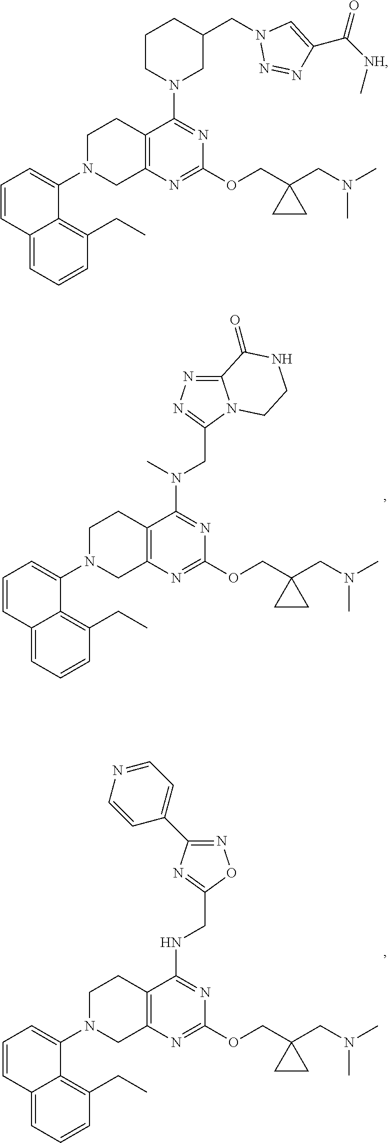 [SMILES: CCc1cccc2cccc(N3CCc4c(nc(OCC5(CN(C)C)CC5)nc4N4CCCC(Cn5cc(C(=O)NC)nn5)C4)C3)c12 ; CCc1cccc2cccc(N3CCc4c(nc(OCC5(CN(C)C)CC5)nc4N(C)Cc4nnc5n4CCNC5=O)C3)c12 ; CCc1cccc2cccc(N3CCc4c(nc(OCC5(CN(C)C)CC5)nc4NCc4nc(-c5ccncc5)no4)C3)c12] 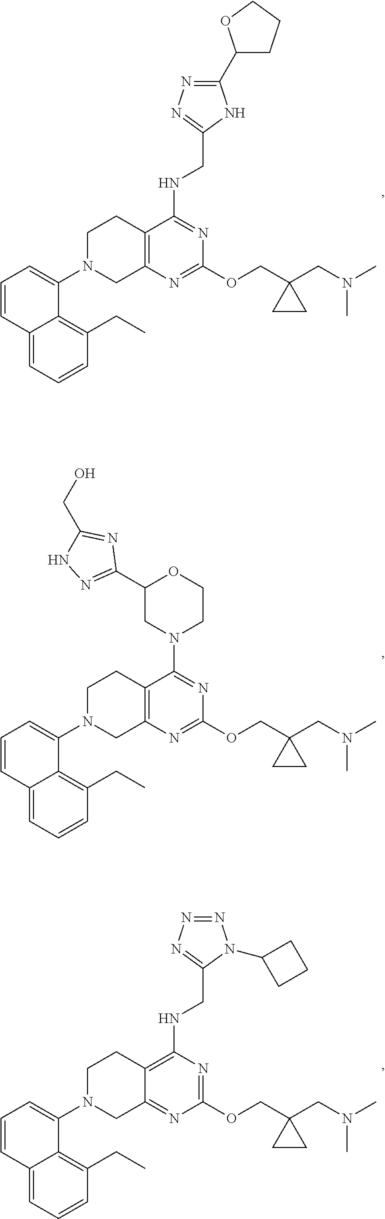 [SMILES: CCc1cccc2cccc(N3CCc4c(nc(OCC5(CN(C)C)CC5)nc4NCc4nnc(C5CCCO5)[nH]4)C3)c12 ; CCc1cccc2cccc(N3CCc4c(nc(OCC5(CN(C)C)CC5)nc4N4CCOC(c5n[nH]c(CO)n5)C4)C3)c12 ; CCc1cccc2cccc(N3CCc4c(nc(OCC5(CN(C)C)CC5)nc4NCc4nnnn4C4CCC4)C3)c12] 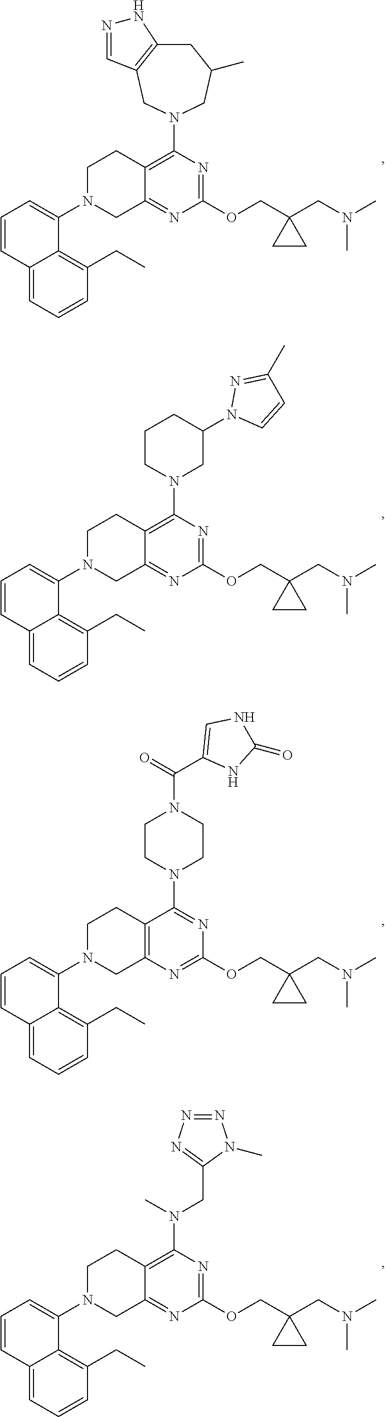 [SMILES: CCc1cccc2cccc(N3CCc4c(nc(OCC5(CN(C)C)CC5)nc4N4Cc5cn[nH]c5CC(C)C4)C3)c12 ; CCc1cccc2cccc(N3CCc4c(nc(OCC5(CN(C)C)CC5)nc4N4CCCC(n5ccc(C)n5)C4)C3)c12 ; CCc1cccc2cccc(N3CCc4c(nc(OCC5(CN(C)C)CC5)nc4N4CCN(C(=O)c5c[nH]c(=O)[nH]5)CC4)C3)c12 ; CCc1cccc2cccc(N3CCc4c(nc(OCC5(CN(C)C)CC5)nc4N(C)Cc4nnnn4C)C3)c12] 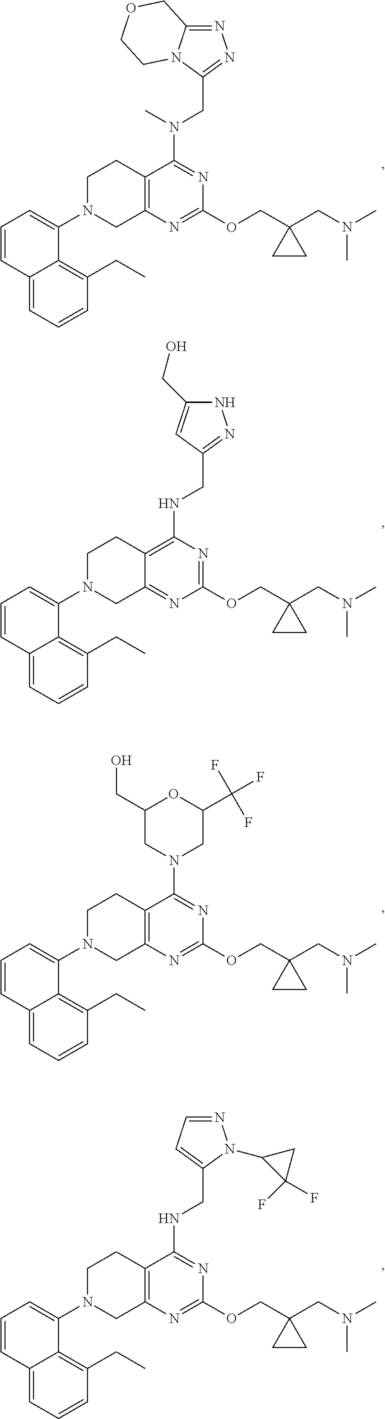 [SMILES: CCc1cccc2cccc(N3CCc4c(nc(OCC5(CN(C)C)CC5)nc4N(C)Cc4nnc5n4CCOC5)C3)c12 ; CCc1cccc2cccc(N3CCc4c(nc(OCC5(CN(C)C)CC5)nc4NCc4cc(CO)[nH]n4)C3)c12 ; CCc1cccc2cccc(N3CCc4c(nc(OCC5(CN(C)C)CC5)nc4N4CC(CO)OC(C(F)(F)F)C4)C3)c12 ; CCc1cccc2cccc(N3CCc4c(nc(OCC5(CN(C)C)CC5)nc4NCc4ccnn4C4CC4(F)F)C3)c12] 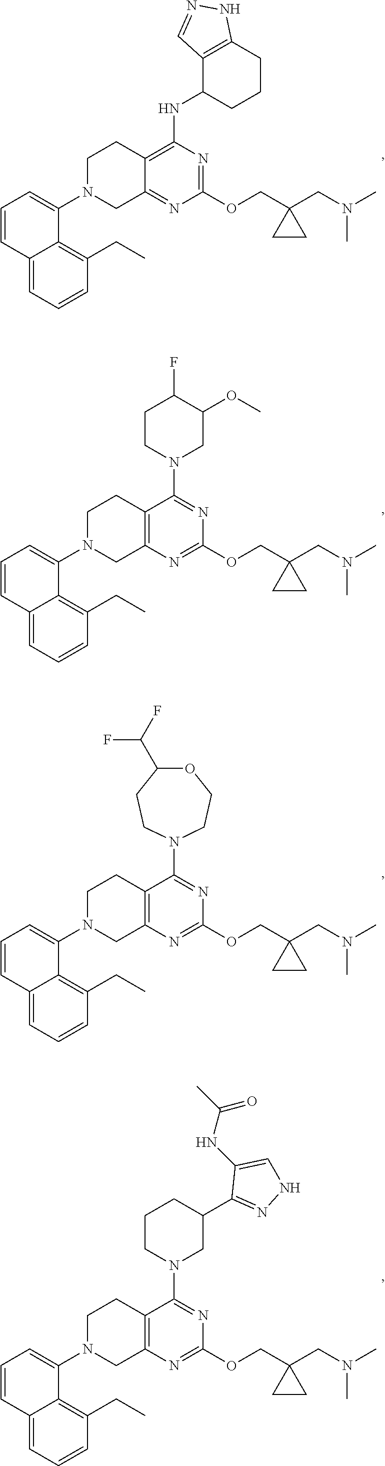 [SMILES: CCc1cccc2cccc(N3CCc4c(nc(OCC5(CN(C)C)CC5)nc4NC4CCCc5[nH]ncc54)C3)c12 ; CCc1cccc2cccc(N3CCc4c(nc(OCC5(CN(C)C)CC5)nc4N4CCC(F)C(OC)C4)C3)c12 ; CCc1cccc2cccc(N3CCc4c(nc(OCC5(CN(C)C)CC5)nc4N4CCOC(C(F)F)CC4)C3)c12 ; CCc1cccc2cccc(N3CCc4c(nc(OCC5(CN(C)C)CC5)nc4N4CCCC(c5n[nH]cc5NC(C)=O)C4)C3)c12] 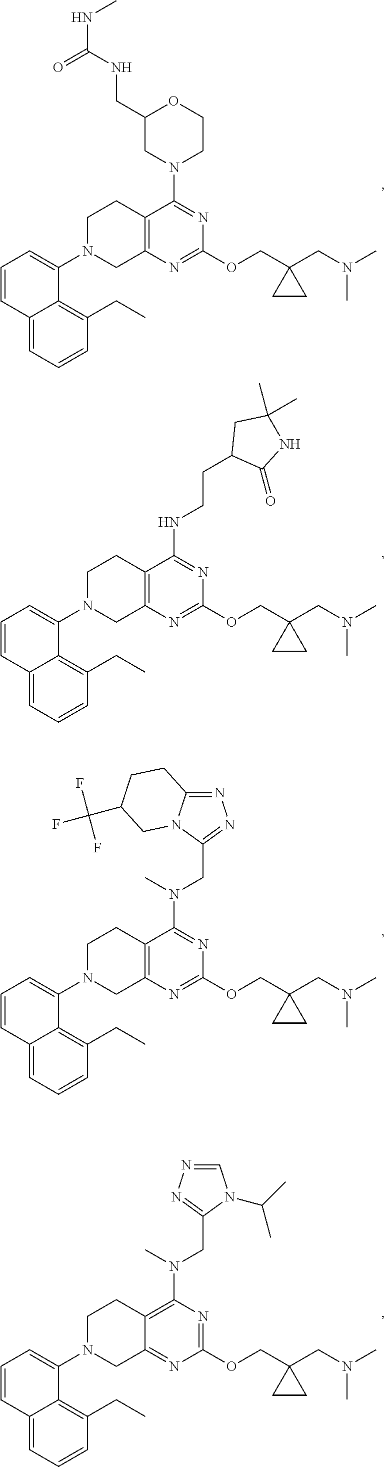 [SMILES: CCc1cccc2cccc(N3CCc4c(nc(OCC5(CN(C)C)CC5)nc4N4CCOC(CNC(=O)NC)C4)C3)c12 ; CCc1cccc2cccc(N3CCc4c(nc(OCC5(CN(C)C)CC5)nc4NCCC4CC(C)(C)NC4=O)C3)c12 ; CCc1cccc2cccc(N3CCc4c(nc(OCC5(CN(C)C)CC5)nc4N(C)Cc4nnc5n4CC(C(F)(F)F)CC5)C3)c12 ; CCc1cccc2cccc(N3CCc4c(nc(OCC5(CN(C)C)CC5)nc4N(C)Cc4nncn4C(C)C)C3)c12] 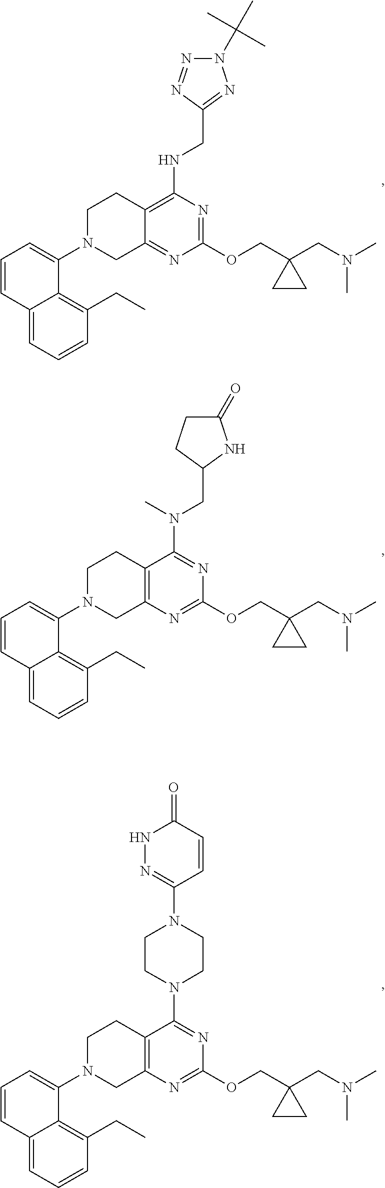 [SMILES: CCc1cccc2cccc(N3CCc4c(nc(OCC5(CN(C)C)CC5)nc4NCc4nnn(C(C)(C)C)n4)C3)c12 ; CCc1cccc2cccc(N3CCc4c(nc(OCC5(CN(C)C)CC5)nc4N(C)CC4CCC(=O)N4)C3)c12 ; CCc1cccc2cccc(N3CCc4c(nc(OCC5(CN(C)C)CC5)nc4N4CCN(c5ccc(=O)[nH]n5)CC4)C3)c12] 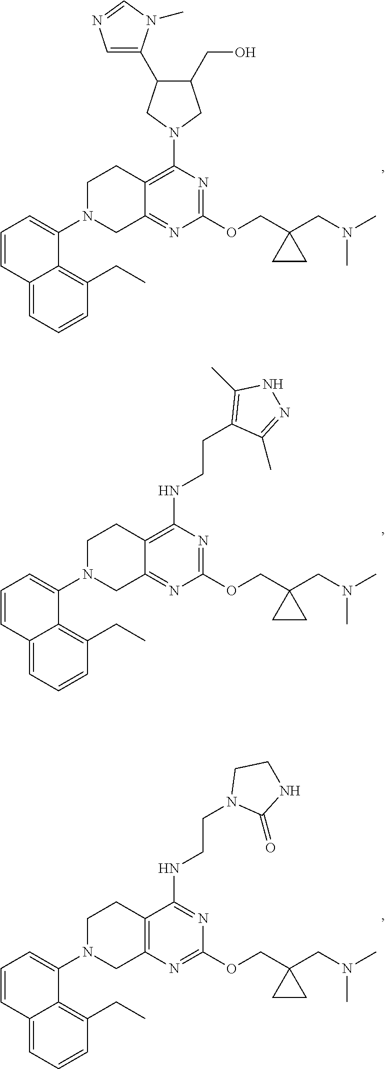 [SMILES: CCc1cccc2cccc(N3CCc4c(nc(OCC5(CN(C)C)CC5)nc4N4CC(CO)C(c5cncn5C)C4)C3)c12 ; CCc1cccc2cccc(N3CCc4c(nc(OCC5(CN(C)C)CC5)nc4NCCc4c(C)n[nH]c4C)C3)c12 ; CCc1cccc2cccc(N3CCc4c(nc(OCC5(CN(C)C)CC5)nc4NCCN4CCNC4=O)C3)c12] 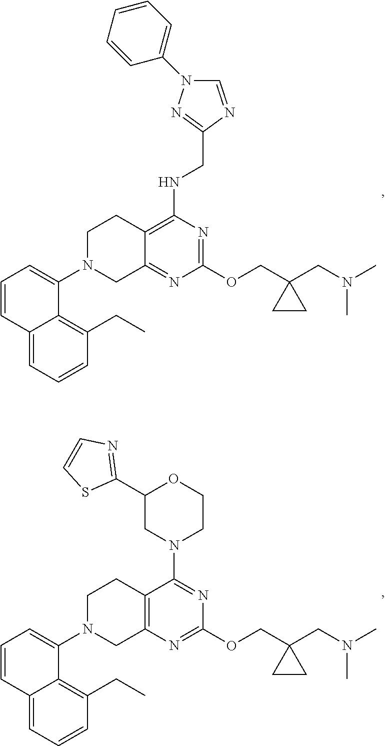 [SMILES: CCc1cccc2cccc(N3CCc4c(nc(OCC5(CN(C)C)CC5)nc4NCc4ncn(-c5ccccc5)n4)C3)c12 ; CCc1cccc2cccc(N3CCc4c(nc(OCC5(CN(C)C)CC5)nc4N4CCOC(c5nccs5)C4)C3)c12] 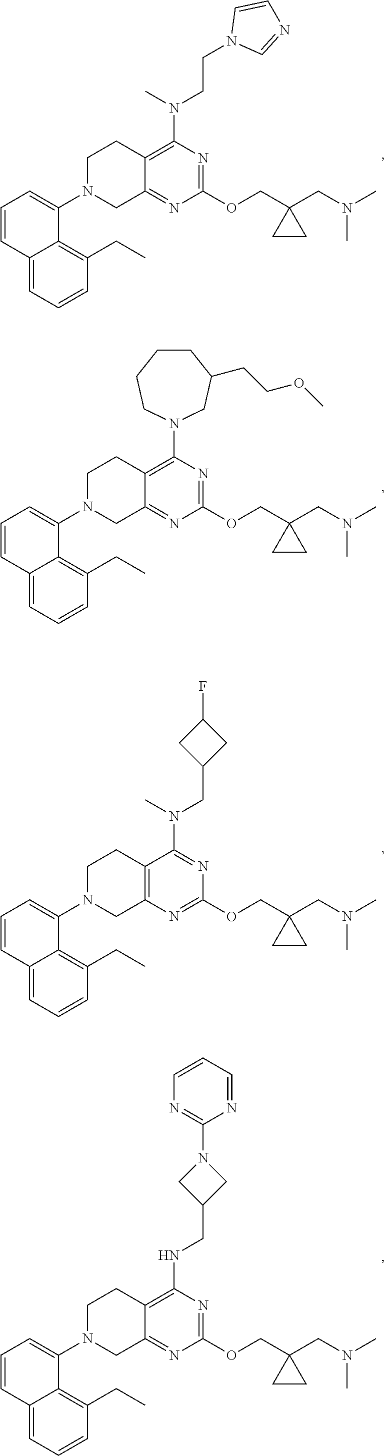 [SMILES: CCc1cccc2cccc(N3CCc4c(nc(OCC5(CN(C)C)CC5)nc4N(C)CCn4ccnc4)C3)c12 ; CCc1cccc2cccc(N3CCc4c(nc(OCC5(CN(C)C)CC5)nc4N4CCCCC(CCOC)C4)C3)c12 ; CCc1cccc2cccc(N3CCc4c(nc(OCC5(CN(C)C)CC5)nc4N(C)CC4CC(F)C4)C3)c12 ; CCc1cccc2cccc(N3CCc4c(nc(OCC5(CN(C)C)CC5)nc4NCC4CN(c5ncccn5)C4)C3)c12] 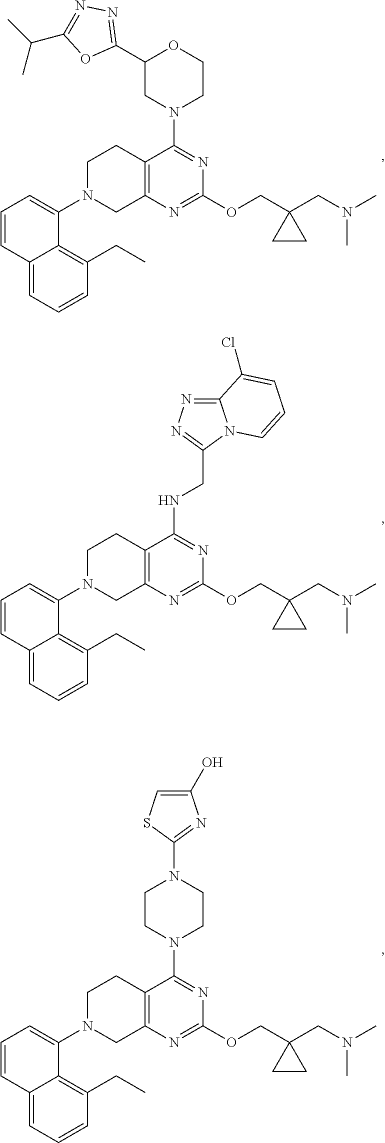 [SMILES: CCc1cccc2cccc(N3CCc4c(nc(OCC5(CN(C)C)CC5)nc4N4CCOC(c5nnc(C(C)C)o5)C4)C3)c12 ; CCc1cccc2cccc(N3CCc4c(nc(OCC5(CN(C)C)CC5)nc4NCc4nnc5c(Cl)cccn45)C3)c12 ; CCc1cccc2cccc(N3CCc4c(nc(OCC5(CN(C)C)CC5)nc4N4CCN(c5nc(O)cs5)CC4)C3)c12] 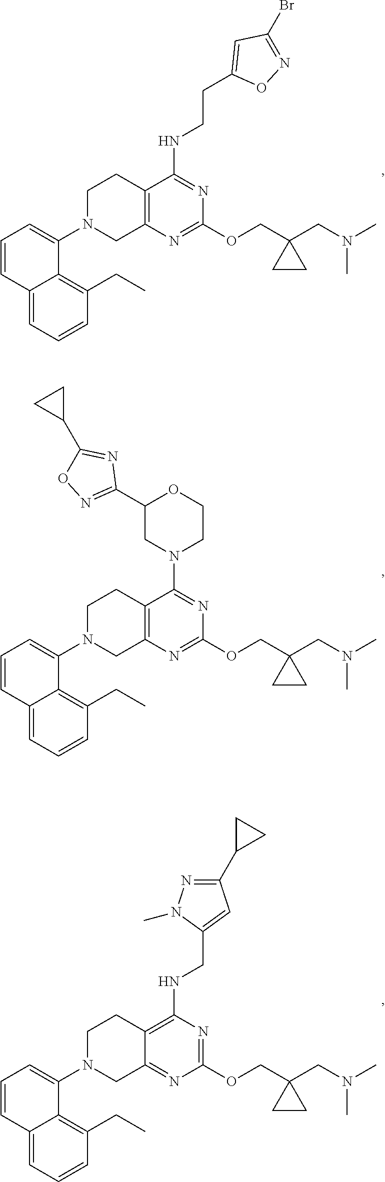 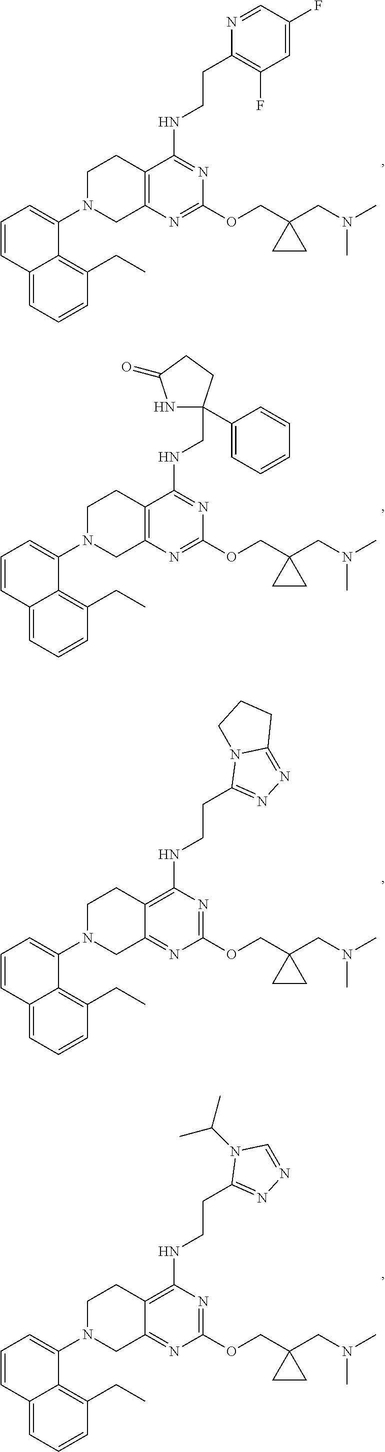 [SMILES: CCc1cccc2cccc(N3CCc4c(nc(OCC5(CN(C)C)CC5)nc4NCCc4ncc(F)cc4F)C3)c12 ; CCc1cccc2cccc(N3CCc4c(nc(OCC5(CN(C)C)CC5)nc4NCC4(c5ccccc5)CCC(=O)N4)C3)c12 ; CCc1cccc2cccc(N3CCc4c(nc(OCC5(CN(C)C)CC5)nc4NCCc4nnc5n4CCC5)C3)c12 ; CCc1cccc2cccc(N3CCc4c(nc(OCC5(CN(C)C)CC5)nc4NCCc4nncn4C(C)C)C3)c12] 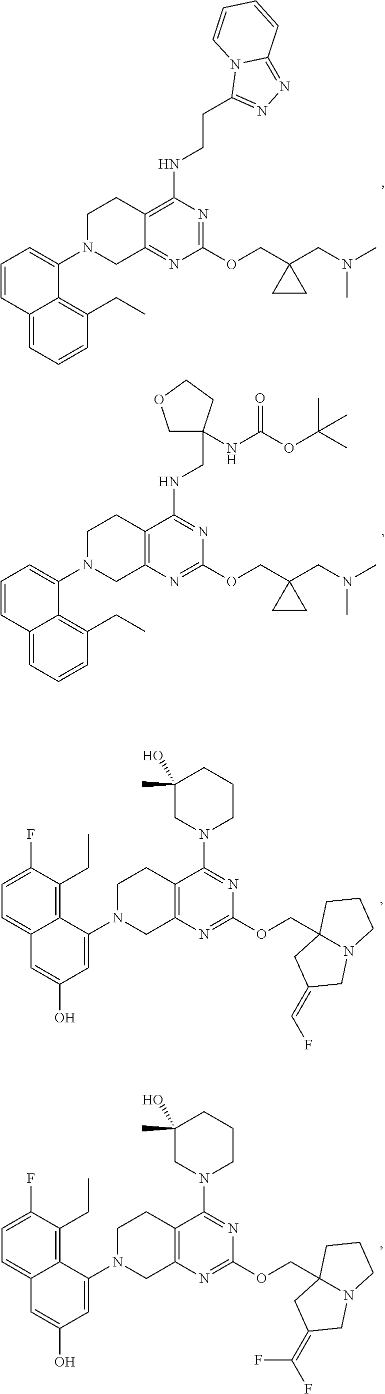 [SMILES: CCc1cccc2cccc(N3CCc4c(nc(OCC5(CN(C)C)CC5)nc4NCCc4nnc5ccccn45)C3)c12 ; CCc1cccc2cccc(N3CCc4c(nc(OCC5(CN(C)C)CC5)nc4NCC4(NC(=O)OC(C)(C)C)CCOC4)C3)c12 ; CCc1c(F)ccc2cc(O)cc(N3CCc4c(nc(OCC56CCCN5C/C(=C\F)C6)nc4N4CCC[C@@](C)(O)C4)C3)c12 ; CCc1c(F)ccc2cc(O)cc(N3CCc4c(nc(OCC56CCCN5CC(=C(F)F)C6)nc4N4CCC[C@@](C)(O)C4)C3)c12] 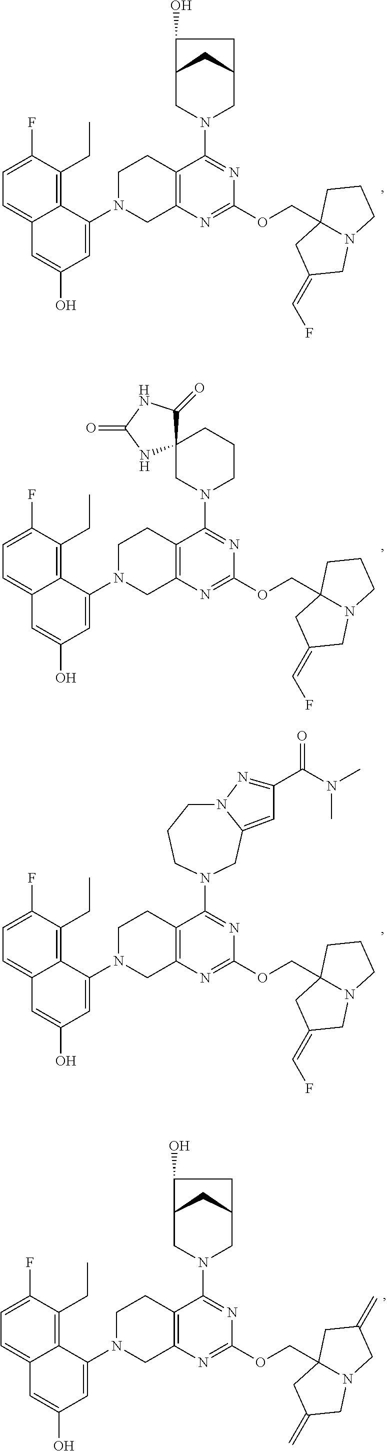 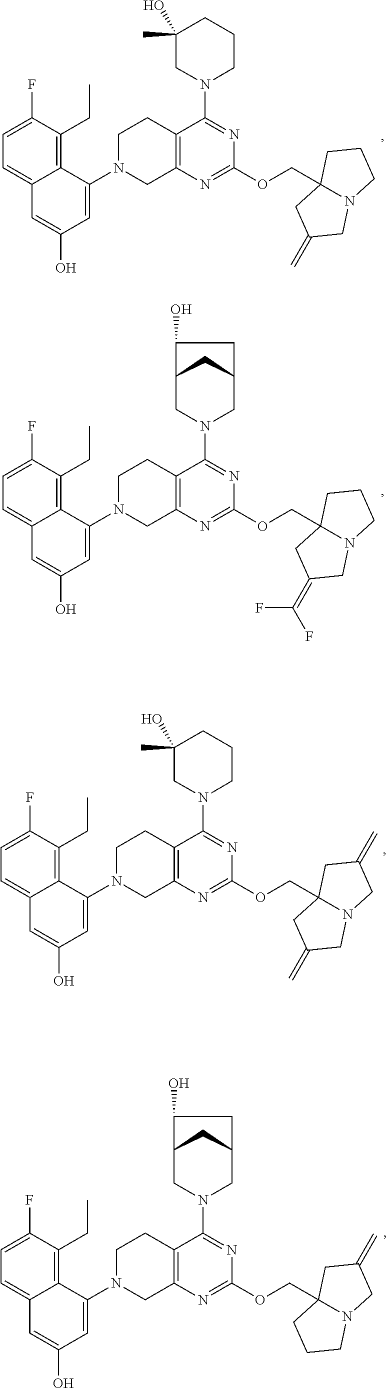 [SMILES: C=C1CN2CCCC2(COc2nc3c(c(N4CCC[C@@](C)(O)C4)n2)CCN(c2cc(O)cc4ccc(F)c(CC)c24)C3)C1 ; CCc1c(F)ccc2cc(O)cc(N3CCc4c(nc(OCC56CCCN5CC(=C(F)F)C6)nc4N4C[C@@H]5C[C@H](C4)[C@H](O)C5)C3)c12 ; C=C1CN2CC(=C)CC2(COc2nc3c(c(N4CCC[C@@](C)(O)C4)n2)CCN(c2cc(O)cc4ccc(F)c(CC)c24)C3)C1 ; C=C1CN2CCCC2(COc2nc3c(c(N4C[C@@H]5C[C@H](C4)[C@H](O)C5)n2)CCN(c2cc(O)cc4ccc(F)c(CC)c24)C3)C1] 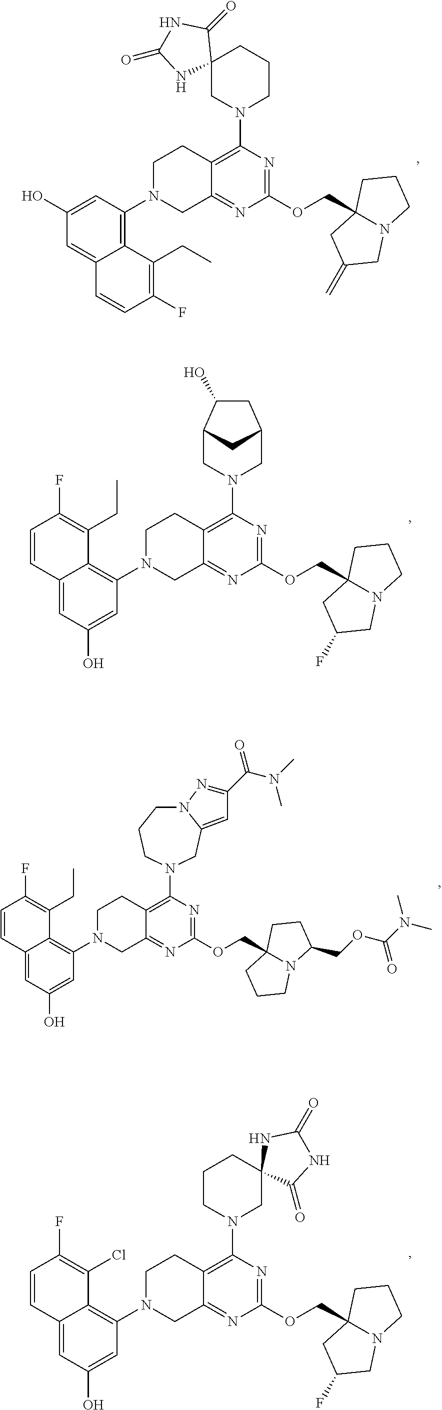 [SMILES: C=C1CN2CCC[C@@]2(COc2nc3c(c(N4CCC[C@]5(C4)NC(=O)NC5=O)n2)CCN(c2cc(O)cc4ccc(F)c(CC)c24)C3)C1 ; CCc1c(F)ccc2cc(O)cc(N3CCc4c(nc(OC[C@@]56CCCN5C[C@H](F)C6)nc4N4C[C@@H]5C[C@H](C4)[C@H](O)C5)C3)c12 ; CCc1c(F)ccc2cc(O)cc(N3CCc4c(nc(OC[C@]56CCCN5[C@H](COC(=O)N(C)C)CC6)nc4N4CCCn5nc(C(=O)N(C)C)cc5C4)C3)c12 ; O=C1NC(=O)[C@]2(CCCN(c3nc(OC[C@@]45CCCN4C[C@H](F)C5)nc4c3CCN(c3cc(O)cc5ccc(F)c(Cl)c35)C4)C2)N1] 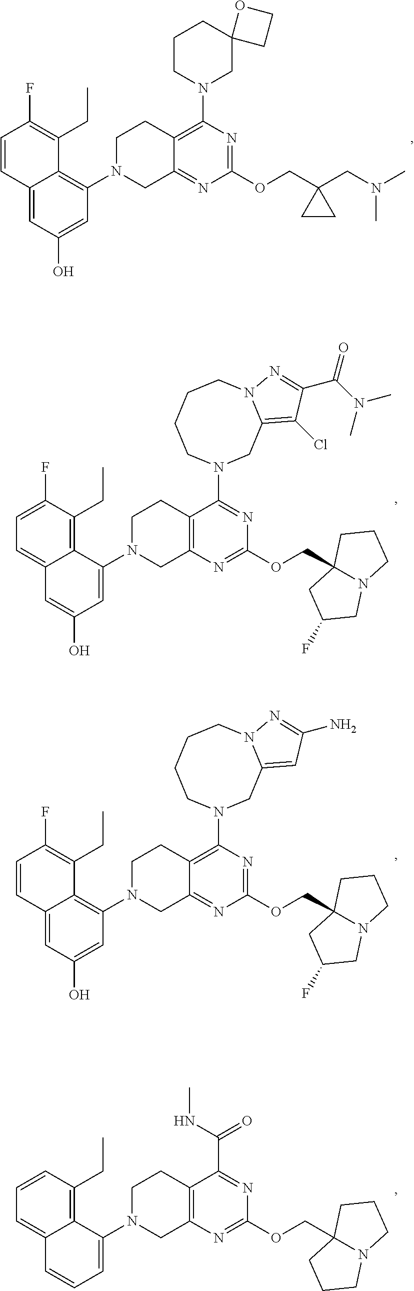 [SMILES: CCc1c(F)ccc2cc(O)cc(N3CCc4c(nc(OCC5(CN(C)C)CC5)nc4N4CCCC5(CCO5)C4)C3)c12 ; CCc1c(F)ccc2cc(O)cc(N3CCc4c(nc(OC[C@@]56CCCN5C[C@H](F)C6)nc4N4CCCCn5nc(C(=O)N(C)C)c(Cl)c5C4)C3)c12 ; CCc1c(F)ccc2cc(O)cc(N3CCc4c(nc(OC[C@@]56CCCN5C[C@H](F)C6)nc4N4CCCCn5nc(N)cc5C4)C3)c12 ; CCc1cccc2cccc(N3CCc4c(nc(OCC56CCCN5CCC6)nc4C(=O)NC)C3)c12] 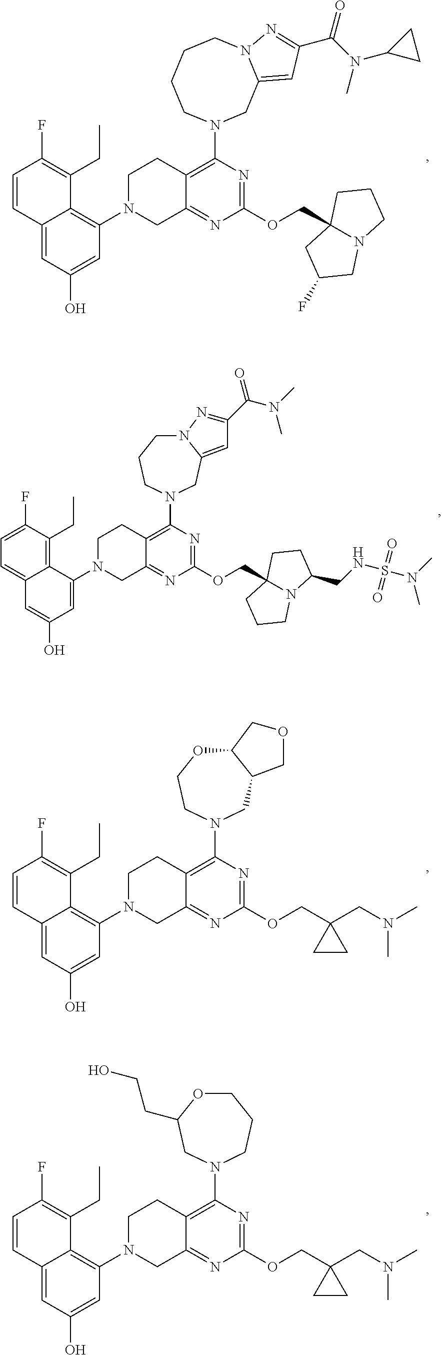 [SMILES: CCc1c(F)ccc2cc(O)cc(N3CCc4c(nc(OC[C@@]56CCCN5C[C@H](F)C6)nc4N4CCCCn5nc(C(=O)N(C)C6CC6)cc5C4)C3)c12 ; CCc1c(F)ccc2cc(O)cc(N3CCc4c(nc(OC[C@]56CCCN5[C@H](CNS(=O)(=O)N(C)C)CC6)nc4N4CCCn5nc(C(=O)N(C)C)cc5C4)C3)c12 ; CCc1c(F)ccc2cc(O)cc(N3CCc4c(nc(OCC5(CN(C)C)CC5)nc4N4CCO[C@@H]5COC[C@@H]5C4)C3)c12 ; CCc1c(F)ccc2cc(O)cc(N3CCc4c(nc(OCC5(CN(C)C)CC5)nc4N4CCCOC(CCO)C4)C3)c12] 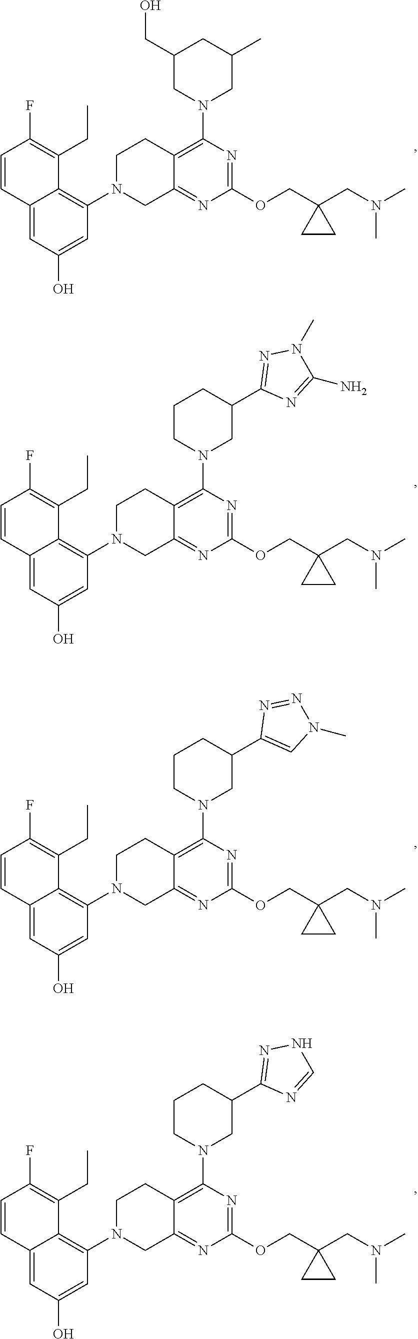 [SMILES: CCc1c(F)ccc2cc(O)cc(N3CCc4c(nc(OCC5(CN(C)C)CC5)nc4N4CC(C)CC(CO)C4)C3)c12 ; CCc1c(F)ccc2cc(O)cc(N3CCc4c(nc(OCC5(CN(C)C)CC5)nc4N4CCCC(c5nc(N)n(C)n5)C4)C3)c12 ; CCc1c(F)ccc2cc(O)cc(N3CCc4c(nc(OCC5(CN(C)C)CC5)nc4N4CCCC(c5cn(C)nn5)C4)C3)c12 ; CCc1c(F)ccc2cc(O)cc(N3CCc4c(nc(OCC5(CN(C)C)CC5)nc4N4CCCC(c5nc[nH]n5)C4)C3)c12] 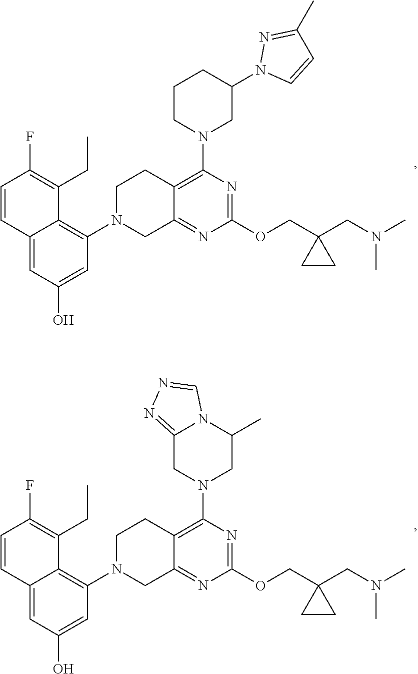 [SMILES: CCc1c(F)ccc2cc(O)cc(N3CCc4c(nc(OCC5(CN(C)C)CC5)nc4N4CCCC(n5ccc(C)n5)C4)C3)c12 ; CCc1c(F)ccc2cc(O)cc(N3CCc4c(nc(OCC5(CN(C)C)CC5)nc4N4Cc5nncn5C(C)C4)C3)c12] 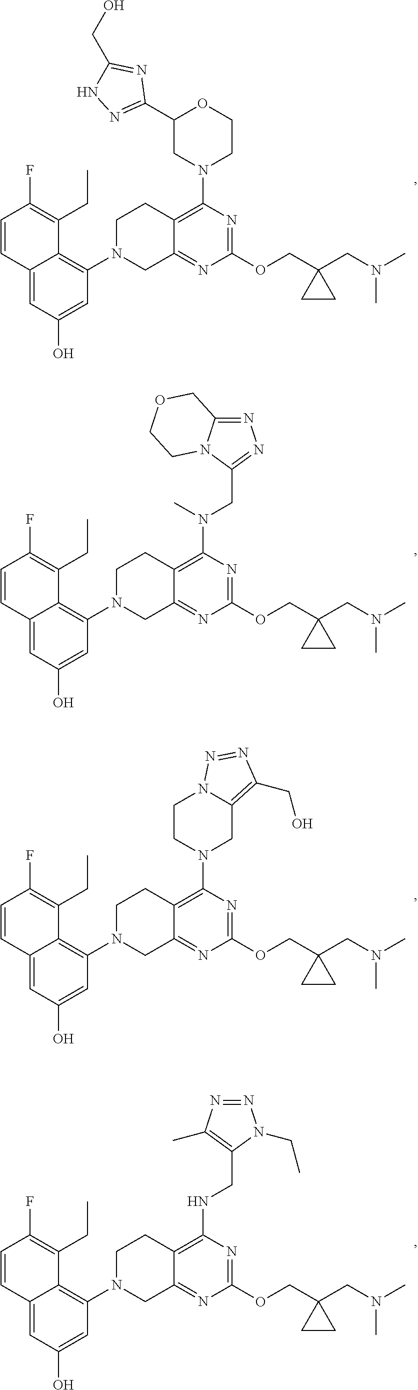 [SMILES: CCc1c(F)ccc2cc(O)cc(N3CCc4c(nc(OCC5(CN(C)C)CC5)nc4N4CCOC(c5n[nH]c(CO)n5)C4)C3)c12 ; CCc1c(F)ccc2cc(O)cc(N3CCc4c(nc(OCC5(CN(C)C)CC5)nc4N(C)Cc4nnc5n4CCOC5)C3)c12 ; CCc1c(F)ccc2cc(O)cc(N3CCc4c(nc(OCC5(CN(C)C)CC5)nc4N4CCn5nnc(CO)c5C4)C3)c12 ; CCc1c(F)ccc2cc(O)cc(N3CCc4c(nc(OCC5(CN(C)C)CC5)nc4NCc4c(C)nnn4CC)C3)c12] 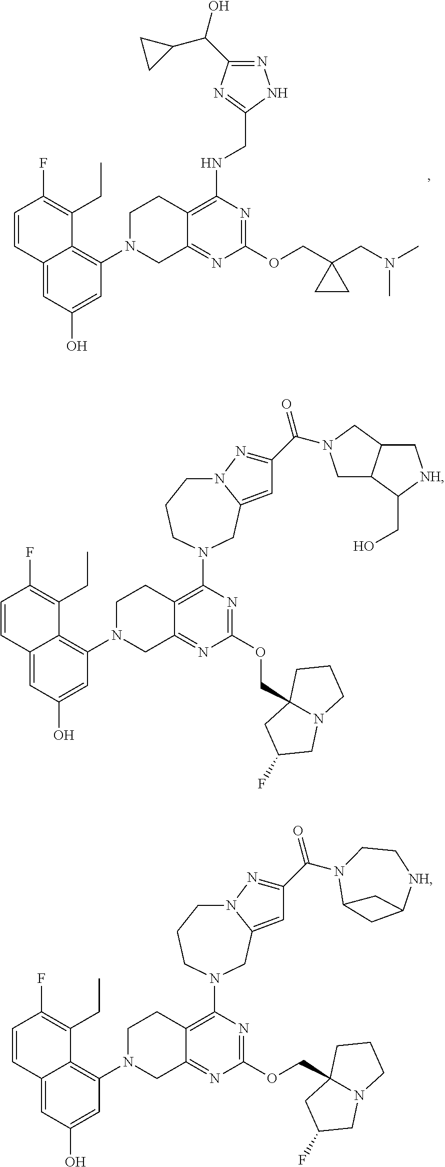 [SMILES: CCc1c(F)ccc2cc(O)cc(N3CCc4c(nc(OCC5(CN(C)C)CC5)nc4NCc4nc(C(O)C5CC5)n[nH]4)C3)c12 ; CCc1c(F)ccc2cc(O)cc(N3CCc4c(nc(OC[C@@]56CCCN5C[C@H](F)C6)nc4N4CCCn5nc(C(=O)N6CC7CNC(CO)C7C6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CCNC7CC6C7)cc5C4)C3)c12] 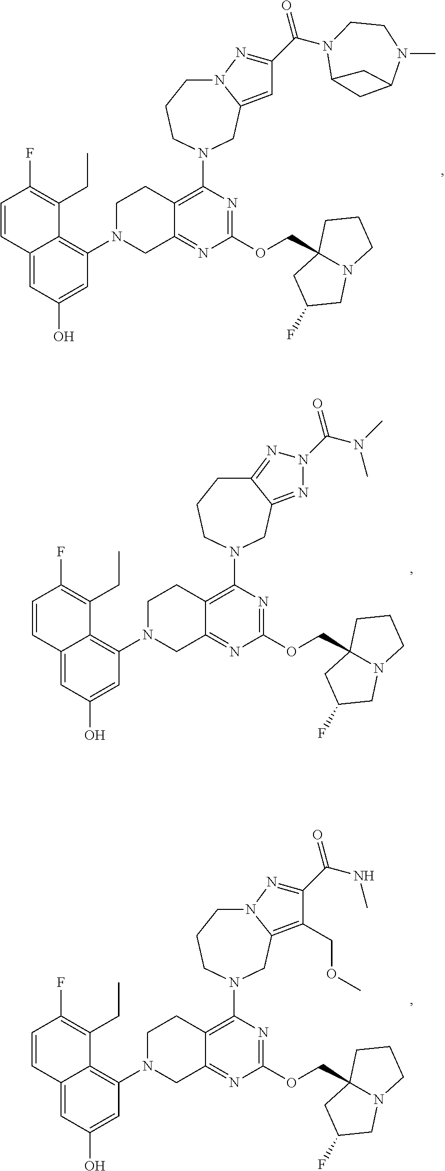 [SMILES: CCc1c(F)ccc2cc(O)cc(N3CCc4c(nc(OC[C@@]56CCCN5C[C@H](F)C6)nc4N4CCCn5nc(C(=O)N6CCN(C)C7CC6C7)cc5C4)C3)c12 ; CCc1c(F)ccc2cc(O)cc(N3CCc4c(nc(OC[C@@]56CCCN5C[C@H](F)C6)nc4N4CCCc5nn(C(=O)N(C)C)nc5C4)C3)c12 ; CCc1c(F)ccc2cc(O)cc(N3CCc4c(nc(OC[C@@]56CCCN5C[C@H](F)C6)nc4N4CCCn5nc(C(=O)NC)c(COC)c5C4)C3)c12] 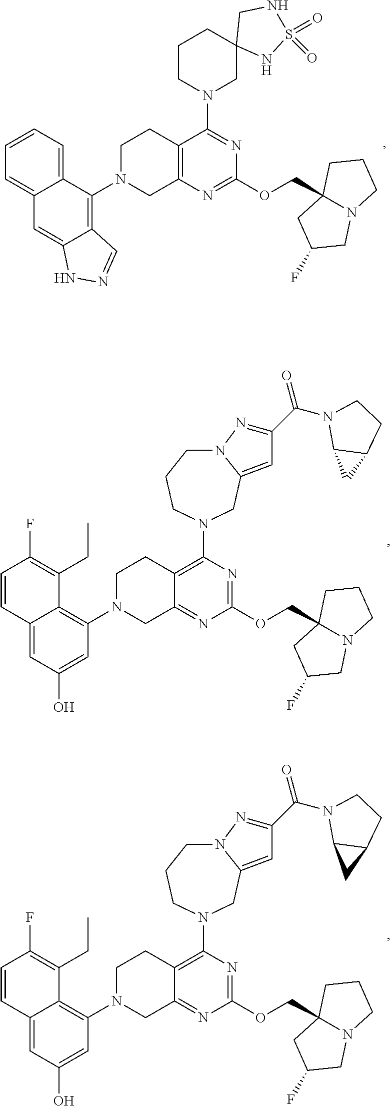 [SMILES: O=S1(=O)NCC2(CCCN(c3nc(OC[C@@]45CCCN4C[C@H](F)C5)nc4c3CCN(c3c5ccccc5cc5[nH]ncc35)C4)C2)N1 ; CCc1c(F)ccc2cc(O)cc(N3CCc4c(nc(OC[C@@]56CCCN5C[C@H](F)C6)nc4N4CCCn5nc(C(=O)N6CC[C@@H]7C[C@@H]76)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC[C@H]7C[C@H]76)cc5C4)C3)c12] 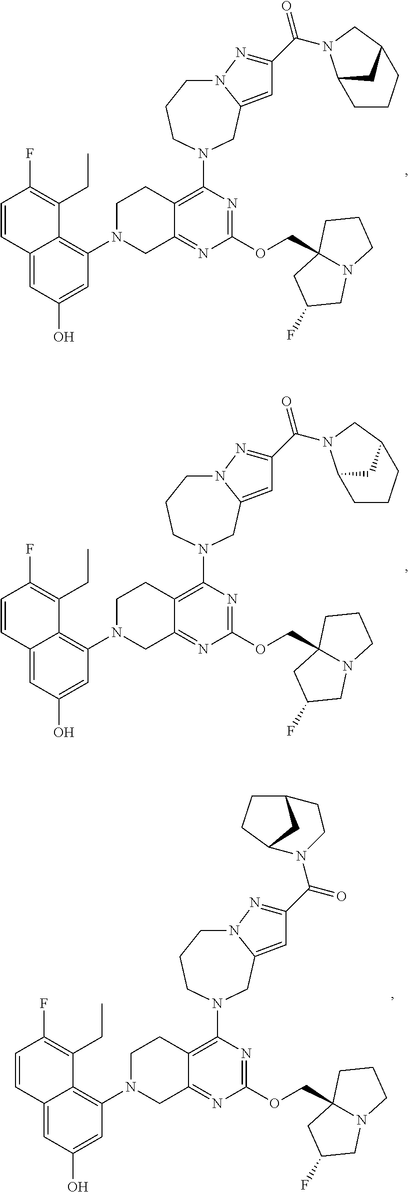 [SMILES: CCc1c(F)ccc2cc(O)cc(N3CCc4c(nc(OC[C@@]56CCCN5C[C@H](F)C6)nc4N4CCCn5nc(C(=O)N6C[C@@H]7CCC[C@H]6C7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6C[C@H]7CCC[C@@H]6C7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC[C@H]7CC[C@@H]6C7)cc5C4)C3)c12] 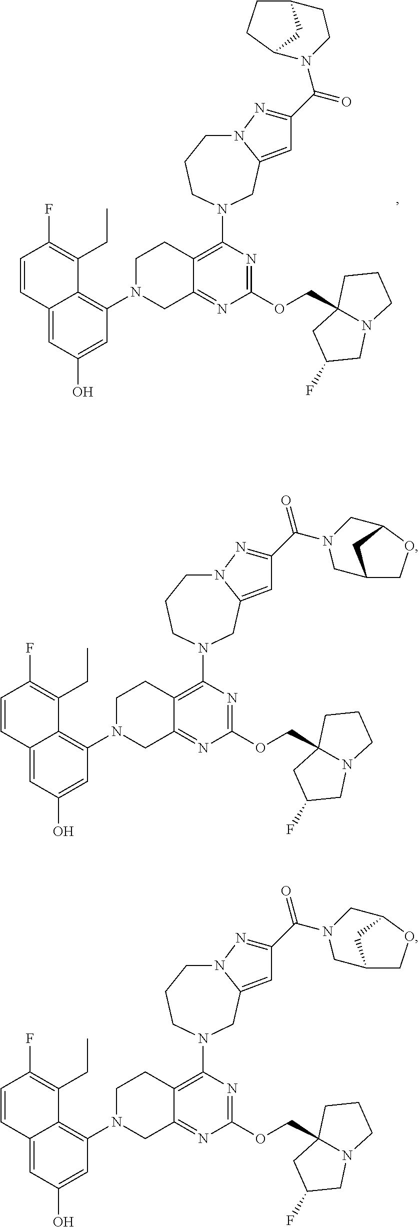 [SMILES: CCc1c(F)ccc2cc(O)cc(N3CCc4c(nc(OC[C@@]56CCCN5C[C@H](F)C6)nc4N4CCCn5nc(C(=O)N6CC[C@@H]7CC[C@H]6C7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6C[C@H]7CO[C@H](C7)C6)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6C[C@@H]7CO[C@@H](C7)C6)cc5C4)C3)c12] 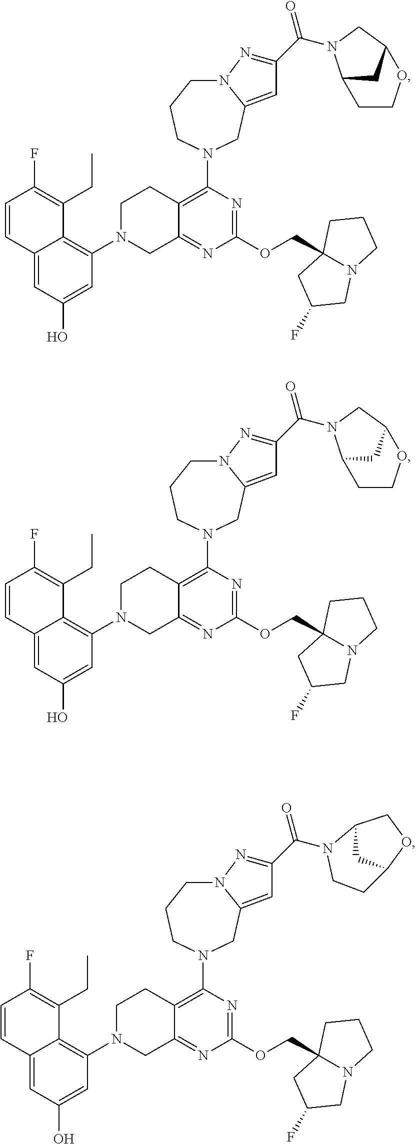 [SMILES: CCc1c(F)ccc2cc(O)cc(N3CCc4c(nc(OC[C@@]56CCCN5C[C@H](F)C6)nc4N4CCCn5nc(C(=O)N6C[C@H]7C[C@@H]6CCO7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6C[C@@H]7C[C@H]6CCO7)cc5C4)C3)c12 ; CCc1c(F)ccc2cc(O)cc(N3CCc4c(nc(OC[C@@]56CCCN5C[C@H](F)C6)nc4N4CCCn5nc(C(=O)N6CC[C@H]7C[C@@H]6CO7)cc5C4)C3)c12] 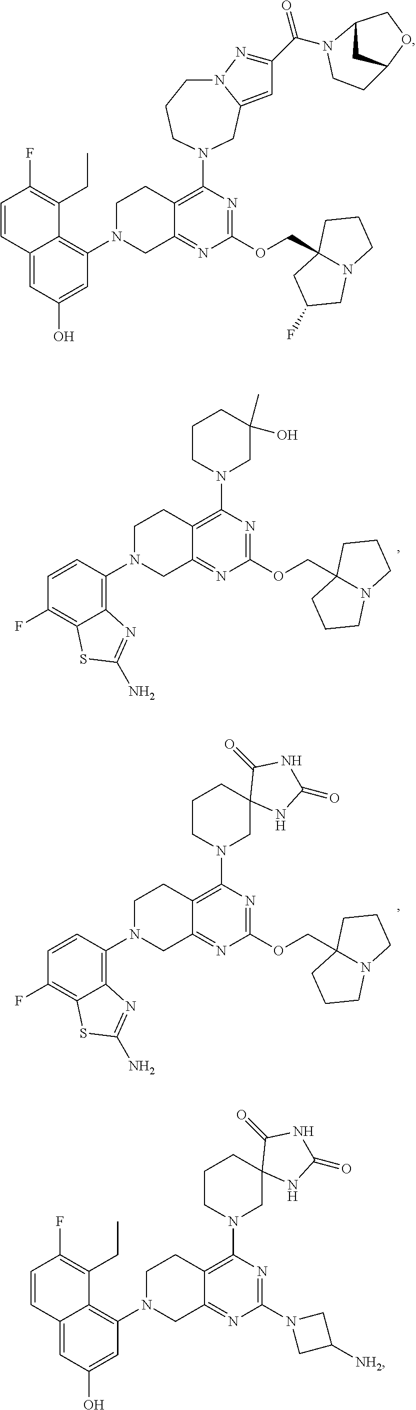 [SMILES: CCc1c(F)ccc2cc(O)cc(N3CCc4c(nc(OC[C@@]56CCCN5C[C@H](F)C6)nc4N4CCCn5nc(C(=O)N6CC[C@@H]7C[C@H]6CO7)cc5C4)C3)c12 ; CC1(O)CCCN(c2nc(OCC34CCCN3CCC4)nc3c2CCN(c2ccc(F)c4sc(N)nc24)C3)C1 ; Nc1nc2c(N3CCc4c(nc(OCC56CCCN5CCC6)nc4N4CCCC5(C4)NC(=O)NC5=O)C3)ccc(F)c2s1 ; CCc1c(F)ccc2cc(O)cc(N3CCc4c(nc(N5CC(N)C5)nc4N4CCCC5(C4)NC(=O)NC5=O)C3)c12] 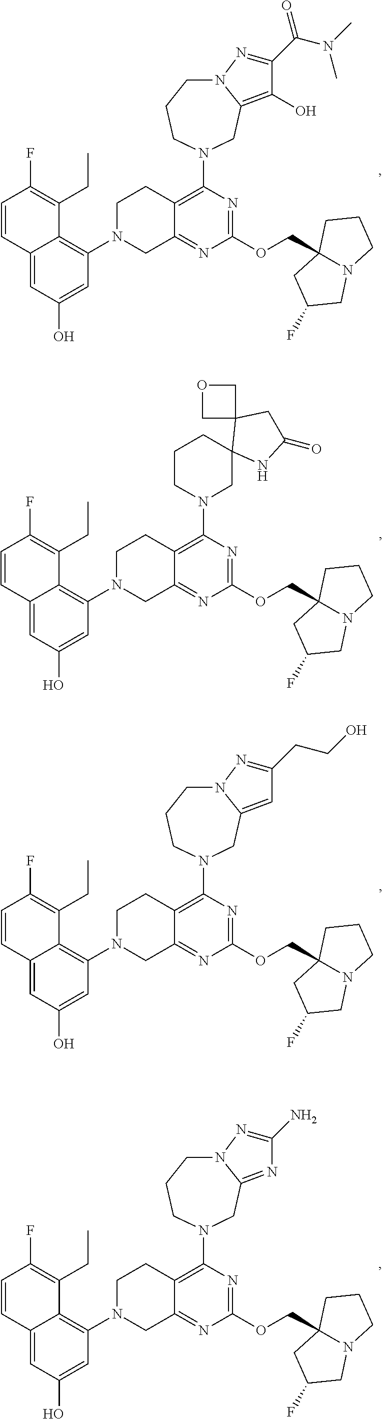 [SMILES: CCc1c(F)ccc2cc(O)cc(N3CCc4c(nc(OC[C@@]56CCCN5C[C@H](F)C6)nc4N4CCCn5nc(C(=O)N(C)C)c(O)c5C4)C3)c12 ; CCc1c(F)ccc2cc(O)cc(N3CCc4c(nc(OC[C@@]56CCCN5C[C@H](F)C6)nc4N4CCCC5(C4)NC(=O)CC54COC4)C3)c12 ; CCc1c(F)ccc2cc(O)cc(N3CCc4c(nc(OC[C@@]56CCCN5C[C@H](F)C6)nc4N4CCCn5nc(CCO)cc5C4)C3)c12 ; CCc1c(F)ccc2cc(O)cc(N3CCc4c(nc(OC[C@@]56CCCN5C[C@H](F)C6)nc4N4CCCn5nc(N)nc5C4)C3)c12] 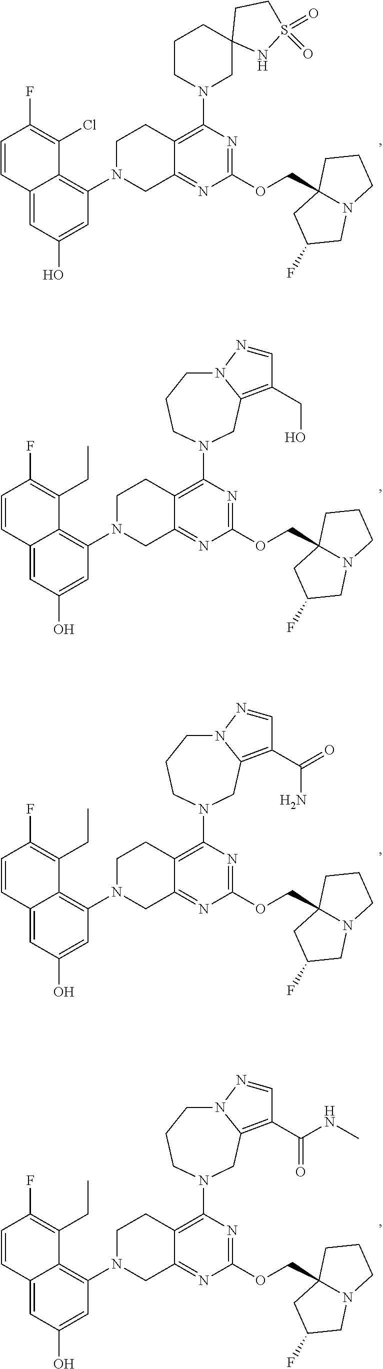 [SMILES: O=S1(=O)CCC2(CCCN(c3nc(OC[C@@]45CCCN4C[C@H](F)C5)nc4c3CCN(c3cc(O)cc5ccc(F)c(Cl)c35)C4)C2)N1 ; CCc1c(F)ccc2cc(O)cc(N3CCc4c(nc(OC[C@@]56CCCN5C[C@H](F)C6)nc4N4CCCn5ncc(CO)c5C4)C3)c12 ; CCc1c(F)ccc2cc(O)cc(N3CCc4c(nc(OC[C@@]56CCCN5C[C@H](F)C6)nc4N4CCCn5ncc(C(N)=O)c5C4)C3)c12 ; CCc1c(F)ccc2cc(O)cc(N3CCc4c(nc(OC[C@@]56CCCN5C[C@H](F)C6)nc4N4CCCn5ncc(C(=O)NC)c5C4)C3)c12] 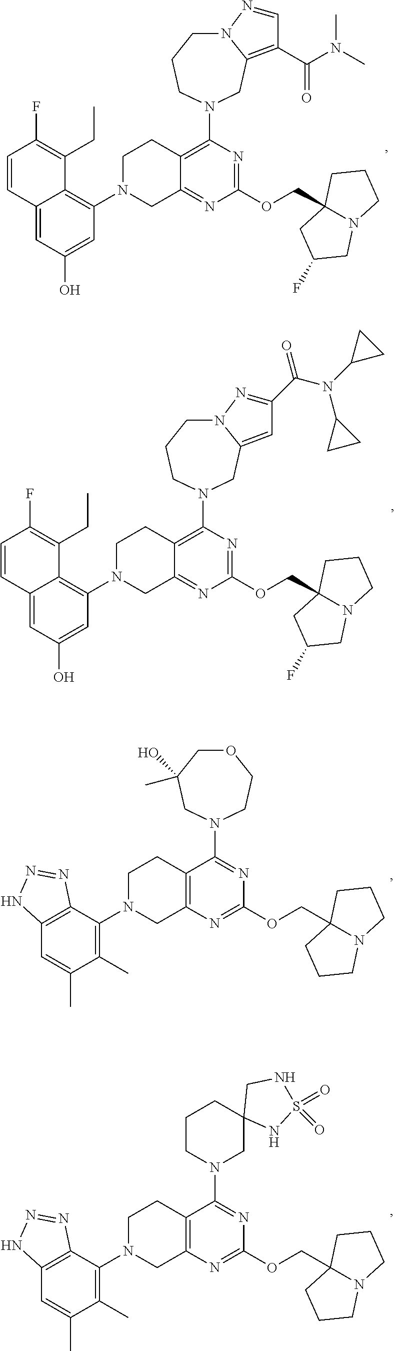 [SMILES: CCc1c(F)ccc2cc(O)cc(N3CCc4c(nc(OC[C@@]56CCCN5C[C@H](F)C6)nc4N4CCCn5ncc(C(=O)N(C)C)c5C4)C3)c12 ; CCc1c(F)ccc2cc(O)cc(N3CCc4c(nc(OC[C@@]56CCCN5C[C@H](F)C6)nc4N4CCCn5nc(C(=O)N(C6CC6)C6CC6)cc5C4)C3)c12 ; Cc1cc2[nH]nnc2c(N2CCc3c(nc(OCC45CCCN4CCC5)nc3N3CCOC[C@@](C)(O)C3)C2)c1C ; Cc1cc2[nH]nnc2c(N2CCc3c(nc(OCC45CCCN4CCC5)nc3N3CCCC4(CNS(=O)(=O)N4)C3)C2)c1C] 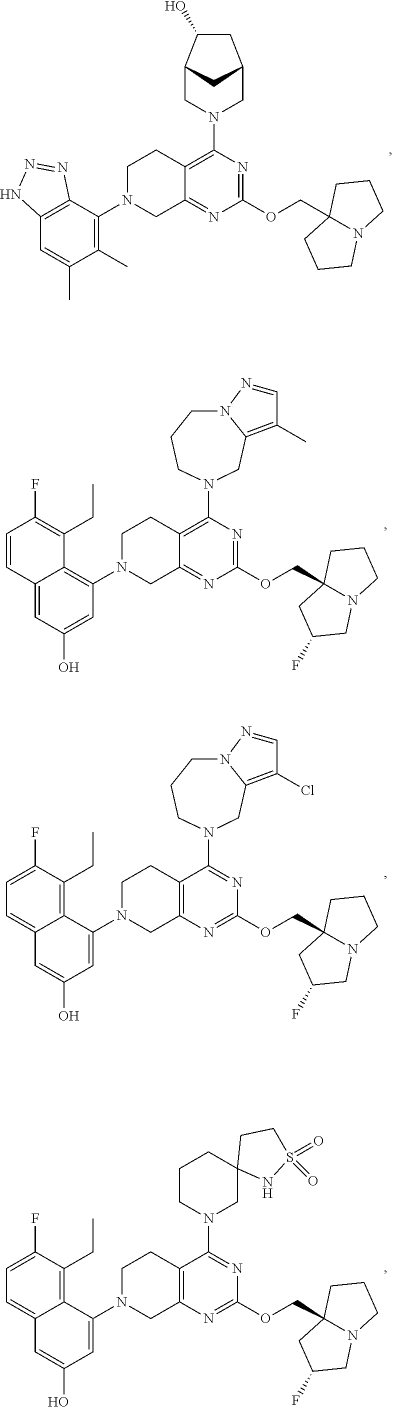 [SMILES: Cc1cc2[nH]nnc2c(N2CCc3c(nc(OCC45CCCN4CCC5)nc3N3C[C@@H]4C[C@H](C3)[C@H](O)C4)C2)c1C ; CCc1c(F)ccc2cc(O)cc(N3CCc4c(nc(OC[C@@]56CCCN5C[C@H](F)C6)nc4N4CCCn5ncc(C)c5C4)C3)c12 ; CCc1c(F)ccc2cc(O)cc(N3CCc4c(nc(OC[C@@]56CCCN5C[C@H](F)C6)nc4N4CCCn5ncc(Cl)c5C4)C3)c12 ; CCc1c(F)ccc2cc(O)cc(N3CCc4c(nc(OC[C@@]56CCCN5C[C@H](F)C6)nc4N4CCCC5(CCS(=O)(=O)N5)C4)C3)c12] 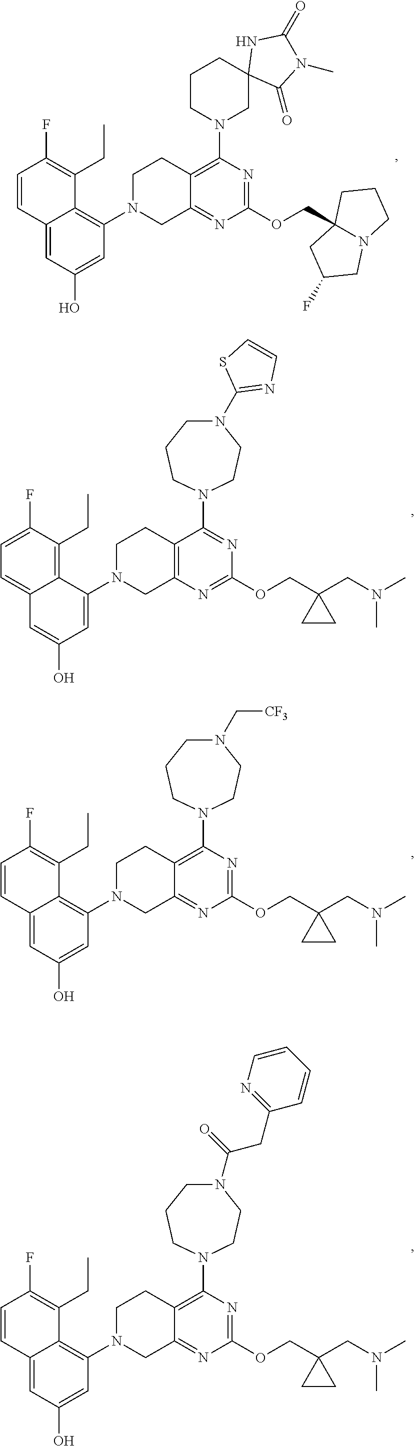 [SMILES: CCc1c(F)ccc2cc(O)cc(N3CCc4c(nc(OC[C@@]56CCCN5C[C@H](F)C6)nc4N4CCCC5(C4)NC(=O)N(C)C5=O)C3)c12 ; CCc1c(F)ccc2cc(O)cc(N3CCc4c(nc(OCC5(CN(C)C)CC5)nc4N4CCCN(c5nccs5)CC4)C3)c12 ; CCc1c(F)ccc2cc(O)cc(N3CCc4c(nc(OCC5(CN(C)C)CC5)nc4N4CCCN(CC(F)(F)F)CC4)C3)c12 ; CCc1c(F)ccc2cc(O)cc(N3CCc4c(nc(OCC5(CN(C)C)CC5)nc4N4CCCN(C(=O)Cc5ccccn5)CC4)C3)c12] 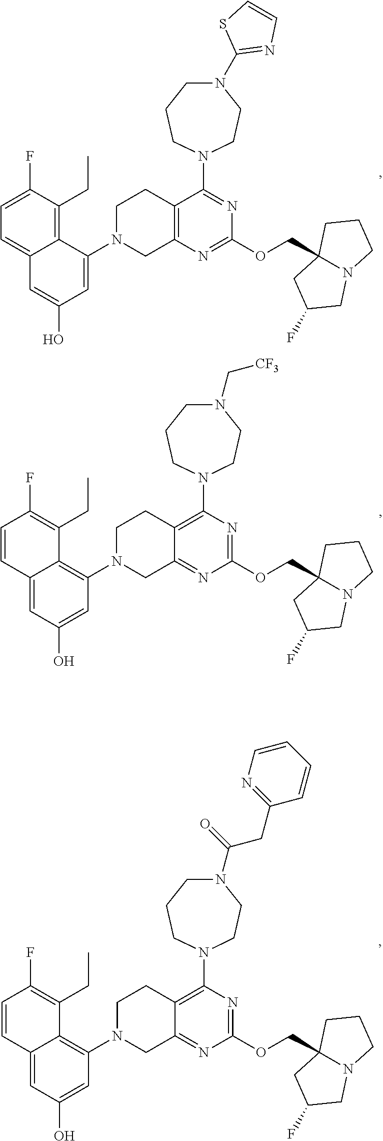 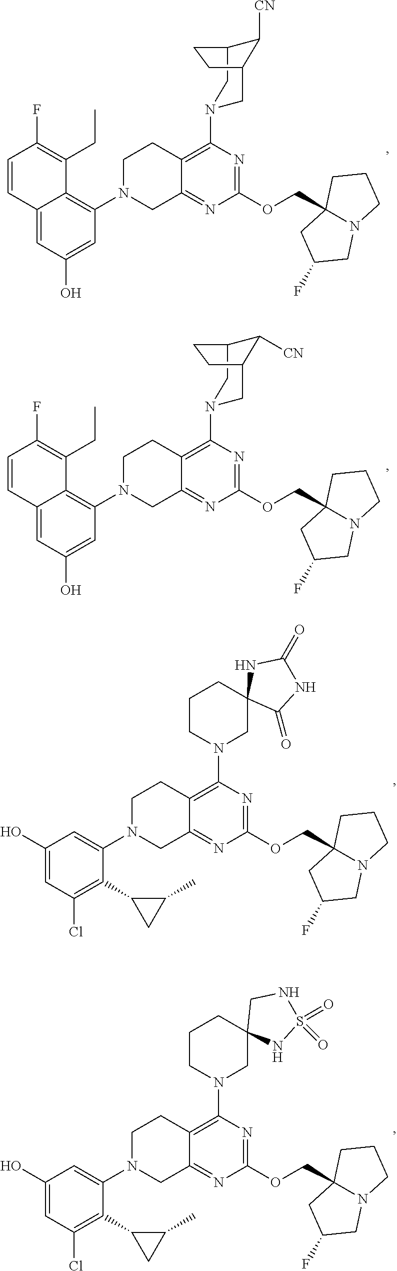 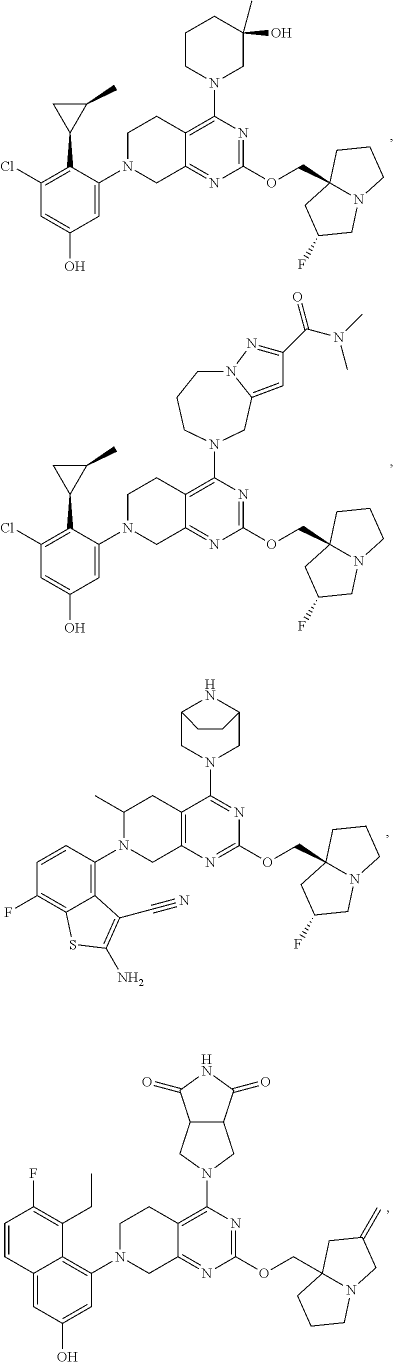 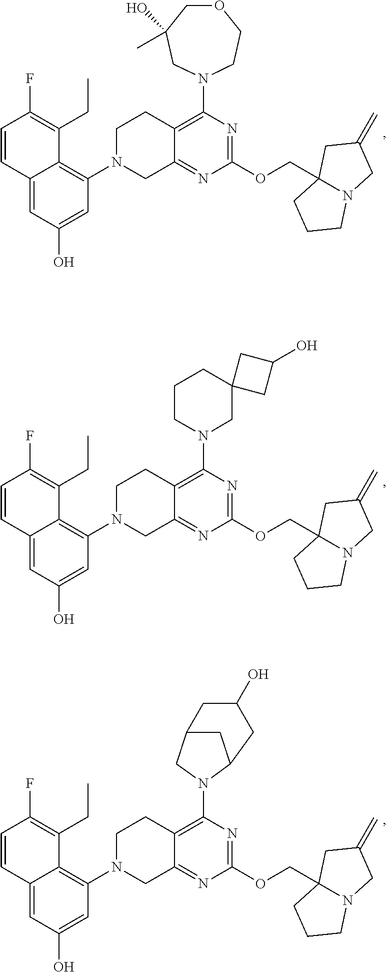 [SMILES: C=C1CN2CCCC2(COc2nc3c(c(N4CCOC[C@@](C)(O)C4)n2)CCN(c2cc(O)cc4ccc(F)c(CC)c24)C3)C1 ; C=C1CN2CCCC2(COc2nc3c(c(N4CCCC5(CC(O)C5)C4)n2)CCN(c2cc(O)cc4ccc(F)c(CC)c24)C3)C1 ; C=C1CN2CCCC2(COc2nc3c(c(N4CC5CC(O)CC4C5)n2)CCN(c2cc(O)cc4ccc(F)c(CC)c24)C3)C1] 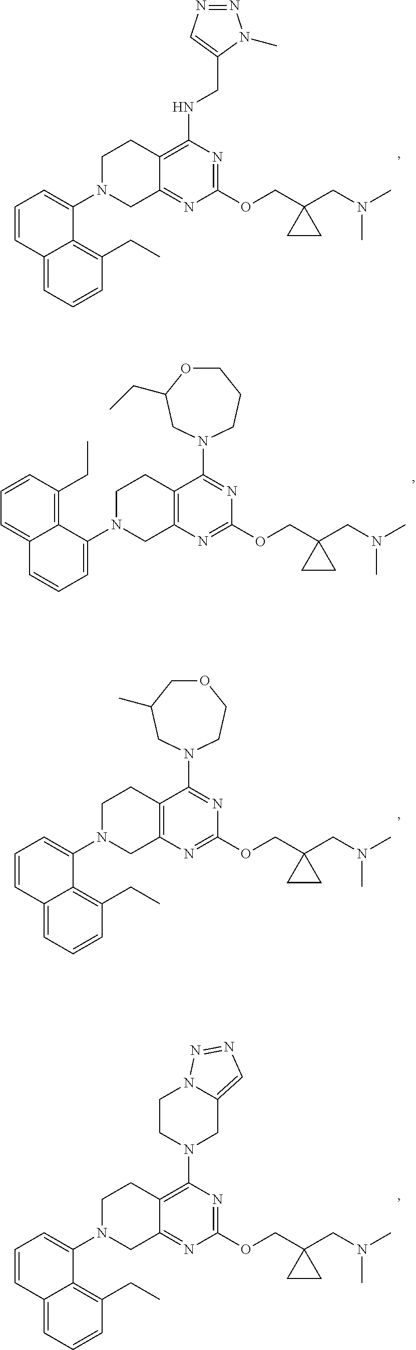 [SMILES: CCc1cccc2cccc(N3CCc4c(nc(OCC5(CN(C)C)CC5)nc4NCc4cnnn4C)C3)c12 ; CCc1cccc2cccc(N3CCc4c(nc(OCC5(CN(C)C)CC5)nc4N4CCCOC(CC)C4)C3)c12 ; CCc1cccc2cccc(N3CCc4c(nc(OCC5(CN(C)C)CC5)nc4N4CCOCC(C)C4)C3)c12 ; CCc1cccc2cccc(N3CCc4c(nc(OCC5(CN(C)C)CC5)nc4N4CCn5nncc5C4)C3)c12] 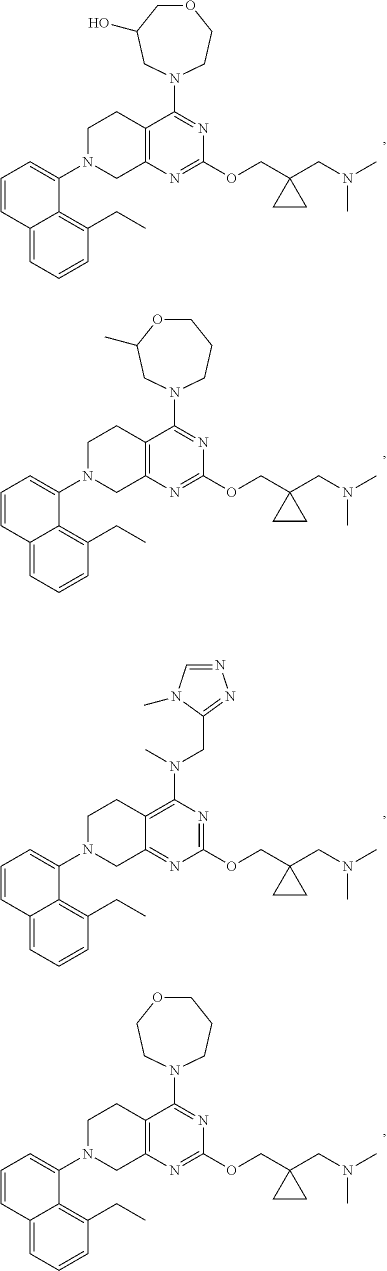 [SMILES: CCc1cccc2cccc(N3CCc4c(nc(OCC5(CN(C)C)CC5)nc4N4CCOCC(O)C4)C3)c12 ; CCc1cccc2cccc(N3CCc4c(nc(OCC5(CN(C)C)CC5)nc4N4CCCOC(C)C4)C3)c12 ; CCc1cccc2cccc(N3CCc4c(nc(OCC5(CN(C)C)CC5)nc4N(C)Cc4nncn4C)C3)c12 ; CCc1cccc2cccc(N3CCc4c(nc(OCC5(CN(C)C)CC5)nc4N4CCCOCC4)C3)c12] 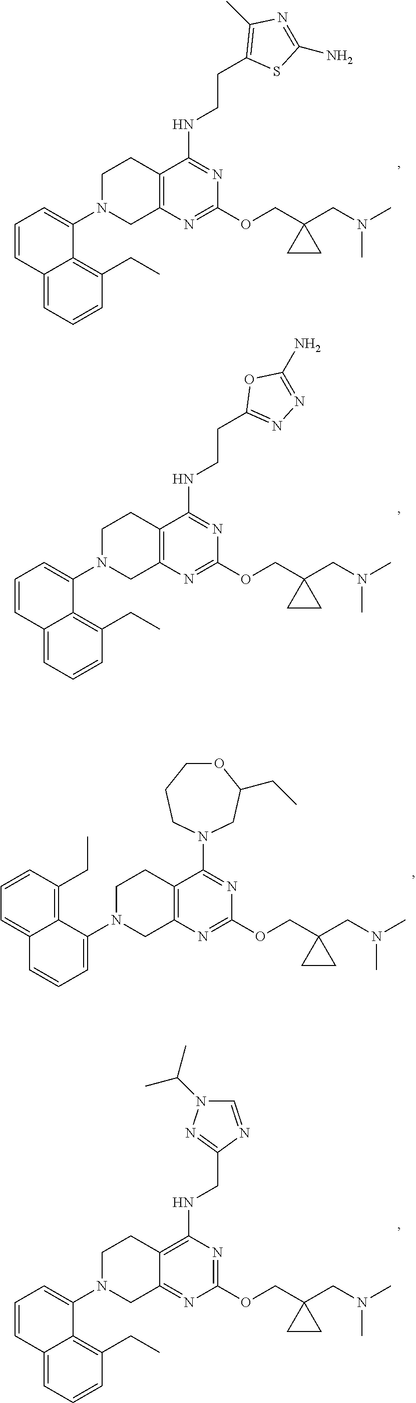 [SMILES: CCc1cccc2cccc(N3CCc4c(nc(OCC5(CN(C)C)CC5)nc4NCCc4sc(N)nc4C)C3)c12 ; CCc1cccc2cccc(N3CCc4c(nc(OCC5(CN(C)C)CC5)nc4NCCc4nnc(N)o4)C3)c12 ; CCc1cccc2cccc(N3CCc4c(nc(OCC5(CN(C)C)CC5)nc4N4CCCOC(CC)C4)C3)c12 ; CCc1cccc2cccc(N3CCc4c(nc(OCC5(CN(C)C)CC5)nc4NCc4ncn(C(C)C)n4)C3)c12] 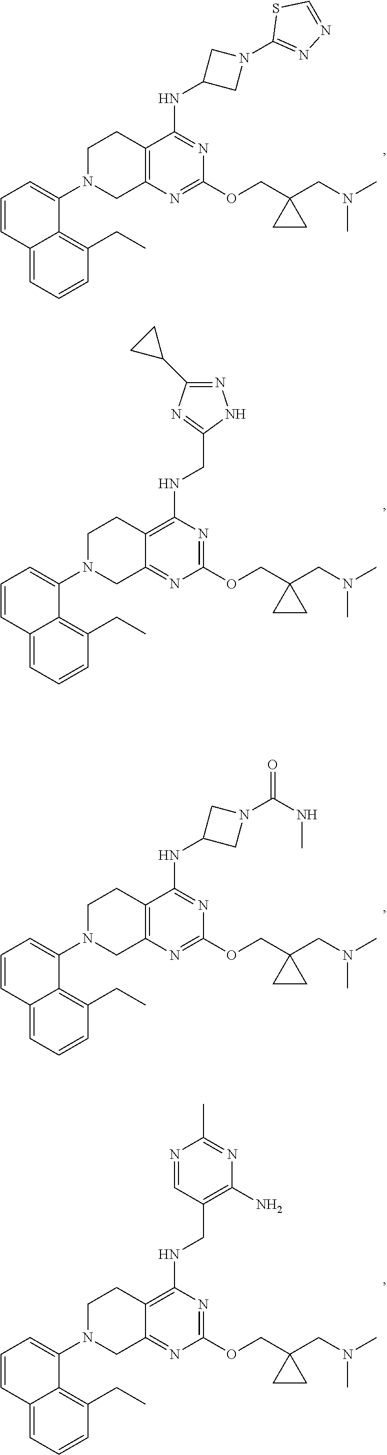 [SMILES: CCc1cccc2cccc(N3CCc4c(nc(OCC5(CN(C)C)CC5)nc4NC4CN(c5nncs5)C4)C3)c12 ; CCc1cccc2cccc(N3CCc4c(nc(OCC5(CN(C)C)CC5)nc4NCc4nc(C5CC5)n[nH]4)C3)c12 ; CCc1cccc2cccc(N3CCc4c(nc(OCC5(CN(C)C)CC5)nc4NC4CN(C(=O)NC)C4)C3)c12 ; CCc1cccc2cccc(N3CCc4c(nc(OCC5(CN(C)C)CC5)nc4NCc4cnc(C)nc4N)C3)c12] 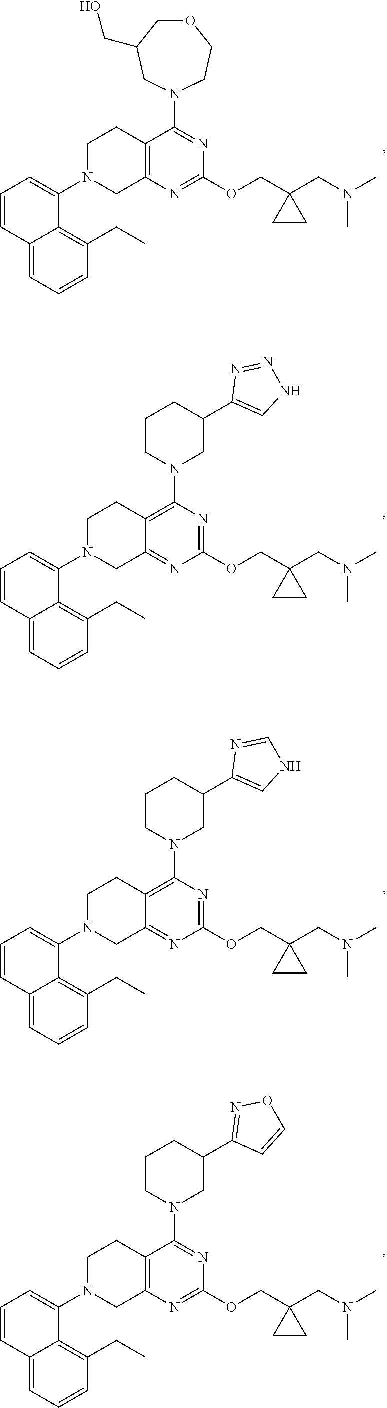 [SMILES: CCc1cccc2cccc(N3CCc4c(nc(OCC5(CN(C)C)CC5)nc4N4CCOCC(CO)C4)C3)c12 ; CCc1cccc2cccc(N3CCc4c(nc(OCC5(CN(C)C)CC5)nc4N4CCCC(c5c[nH]nn5)C4)C3)c12 ; CCc1cccc2cccc(N3CCc4c(nc(OCC5(CN(C)C)CC5)nc4N4CCCC(c5c[nH]cn5)C4)C3)c12 ; CCc1cccc2cccc(N3CCc4c(nc(OCC5(CN(C)C)CC5)nc4N4CCCC(c5ccon5)C4)C3)c12] 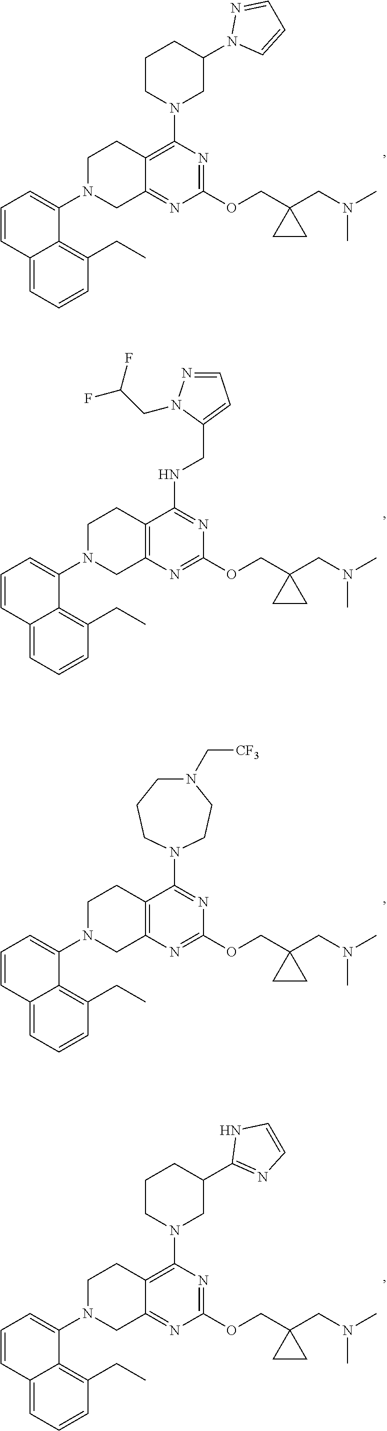 [SMILES: CCc1cccc2cccc(N3CCc4c(nc(OCC5(CN(C)C)CC5)nc4N4CCCC(n5cccn5)C4)C3)c12 ; CCc1cccc2cccc(N3CCc4c(nc(OCC5(CN(C)C)CC5)nc4NCc4ccnn4CC(F)F)C3)c12 ; CCc1cccc2cccc(N3CCc4c(nc(OCC5(CN(C)C)CC5)nc4N4CCCN(CC(F)(F)F)CC4)C3)c12 ; CCc1cccc2cccc(N3CCc4c(nc(OCC5(CN(C)C)CC5)nc4N4CCCC(c5ncc[nH]5)C4)C3)c12] 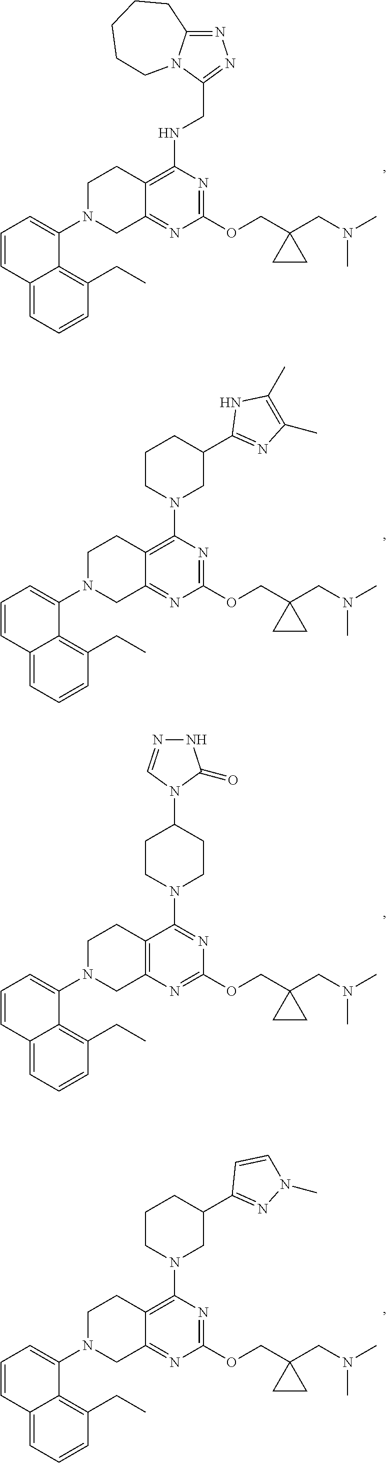 [SMILES: CCc1cccc2cccc(N3CCc4c(nc(OCC5(CN(C)C)CC5)nc4NCc4nnc5n4CCCCC5)C3)c12 ; CCc1cccc2cccc(N3CCc4c(nc(OCC5(CN(C)C)CC5)nc4N4CCCC(c5nc(C)c(C)[nH]5)C4)C3)c12 ; CCc1cccc2cccc(N3CCc4c(nc(OCC5(CN(C)C)CC5)nc4N4CCC(n5cn[nH]c5=O)CC4)C3)c12 ; CCc1cccc2cccc(N3CCc4c(nc(OCC5(CN(C)C)CC5)nc4N4CCCC(c5ccn(C)n5)C4)C3)c12] 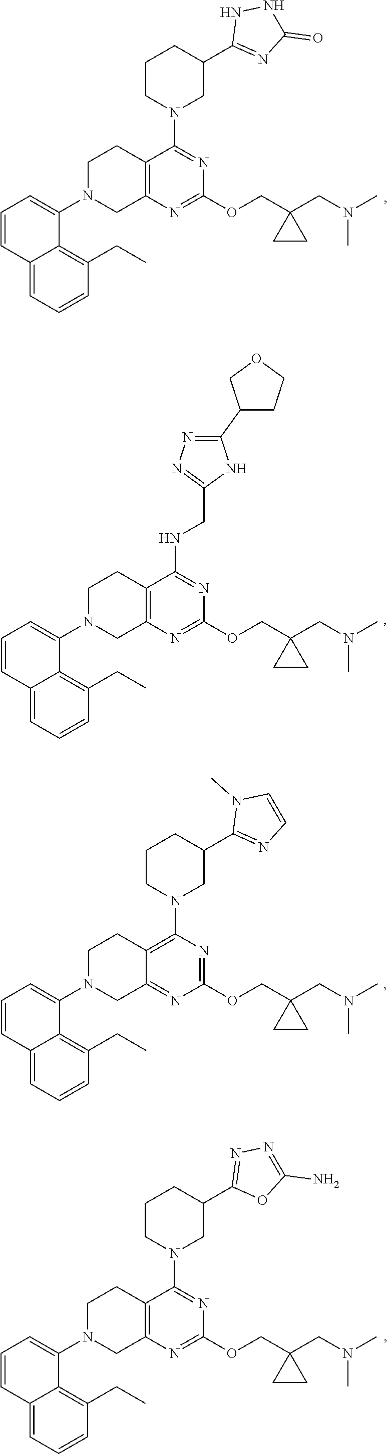 [SMILES: CCc1cccc2cccc(N3CCc4c(nc(OCC5(CN(C)C)CC5)nc4N4CCCC(c5nc(=O)[nH][nH]5)C4)C3)c12 ; CCc1cccc2cccc(N3CCc4c(nc(OCC5(CN(C)C)CC5)nc4NCc4nnc(C5CCOC5)[nH]4)C3)c12 ; CCc1cccc2cccc(N3CCc4c(nc(OCC5(CN(C)C)CC5)nc4N4CCCC(c5nccn5C)C4)C3)c12 ; CCc1cccc2cccc(N3CCc4c(nc(OCC5(CN(C)C)CC5)nc4N4CCCC(c5nnc(N)o5)C4)C3)c12] 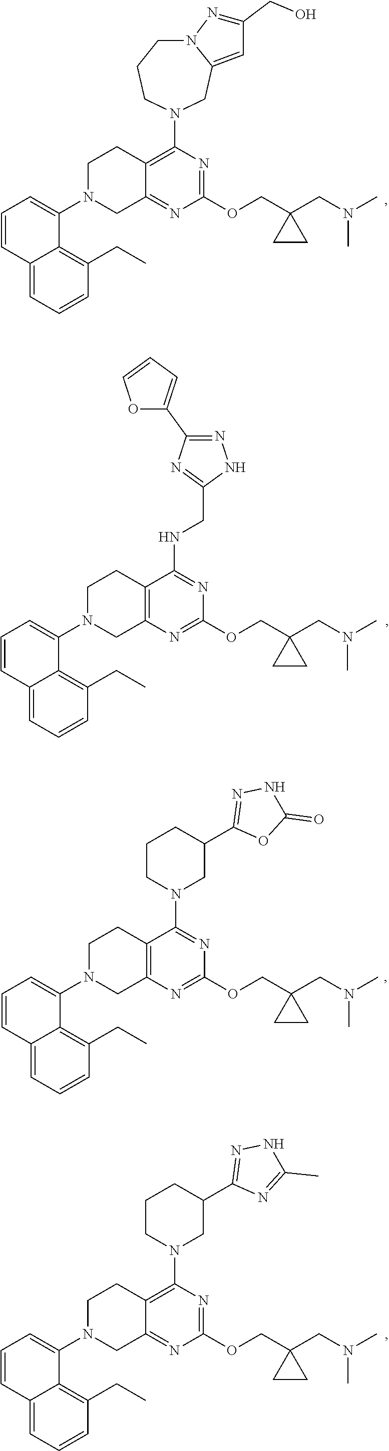 [SMILES: CCc1cccc2cccc(N3CCc4c(nc(OCC5(CN(C)C)CC5)nc4N4CCCn5nc(CO)cc5C4)C3)c12 ; CCc1cccc2cccc(N3CCc4c(nc(OCC5(CN(C)C)CC5)nc4NCc4nc(-c5ccco5)n[nH]4)C3)c12 ; CCc1cccc2cccc(N3CCc4c(nc(OCC5(CN(C)C)CC5)nc4N4CCCC(c5n[nH]c(=O)o5)C4)C3)c12 ; CCc1cccc2cccc(N3CCc4c(nc(OCC5(CN(C)C)CC5)nc4N4CCCC(c5n[nH]c(C)n5)C4)C3)c12] 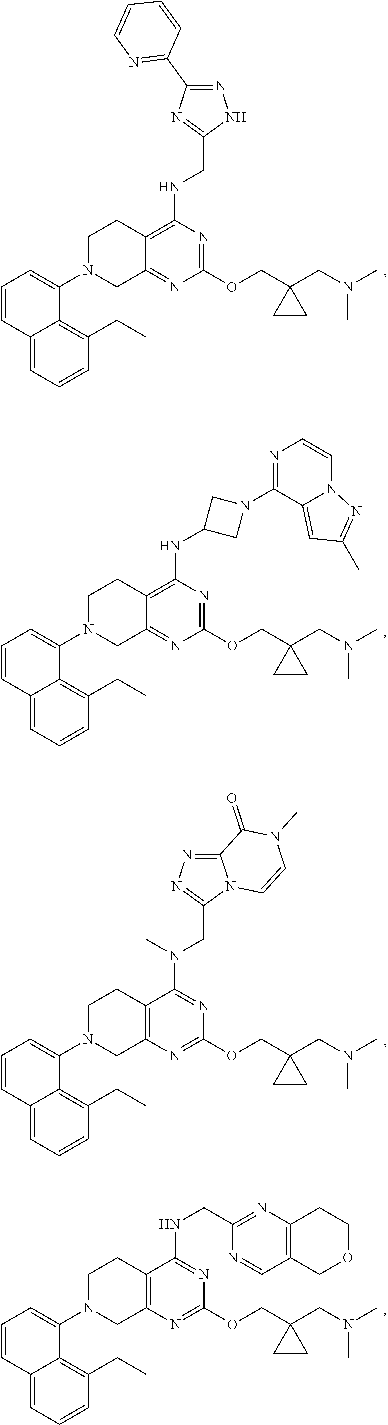 [SMILES: CCc1cccc2cccc(N3CCc4c(nc(OCC5(CN(C)C)CC5)nc4NCc4nc(-c5ccccn5)n[nH]4)C3)c12 ; CCc1cccc2cccc(N3CCc4c(nc(OCC5(CN(C)C)CC5)nc4NC4CN(c5nccn6nc(C)cc56)C4)C3)c12 ; CCc1cccc2cccc(N3CCc4c(nc(OCC5(CN(C)C)CC5)nc4N(C)Cc4nnc5c(=O)n(C)ccn45)C3)c12 ; CCc1cccc2cccc(N3CCc4c(nc(OCC5(CN(C)C)CC5)nc4NCc4ncc5c(n4)CCOC5)C3)c12] 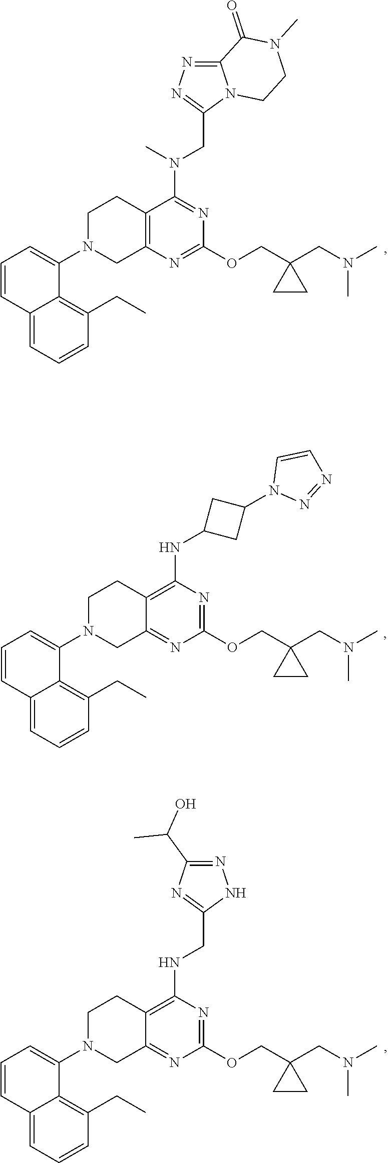 [SMILES: CCc1cccc2cccc(N3CCc4c(nc(OCC5(CN(C)C)CC5)nc4N(C)Cc4nnc5n4CCN(C)C5=O)C3)c12 ; CCc1cccc2cccc(N3CCc4c(nc(OCC5(CN(C)C)CC5)nc4NC4CC(n5ccnn5)C4)C3)c12 ; CCc1cccc2cccc(N3CCc4c(nc(OCC5(CN(C)C)CC5)nc4NCc4nc(C(C)O)n[nH]4)C3)c12] 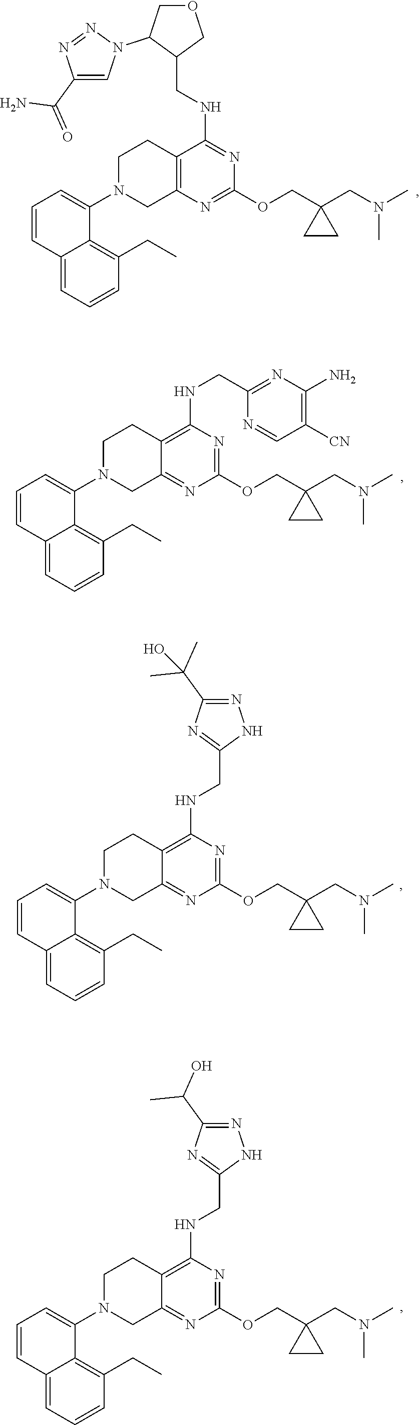 [SMILES: CCc1cccc2cccc(N3CCc4c(nc(OCC5(CN(C)C)CC5)nc4NCC4COCC4n4cc(C(N)=O)nn4)C3)c12 ; CCc1cccc2cccc(N3CCc4c(nc(OCC5(CN(C)C)CC5)nc4NCc4ncc(C#N)c(N)n4)C3)c12 ; CCc1cccc2cccc(N3CCc4c(nc(OCC5(CN(C)C)CC5)nc4NCc4nc(C(C)(C)O)n[nH]4)C3)c12 ; CCc1cccc2cccc(N3CCc4c(nc(OCC5(CN(C)C)CC5)nc4NCc4nc(C(C)O)n[nH]4)C3)c12] 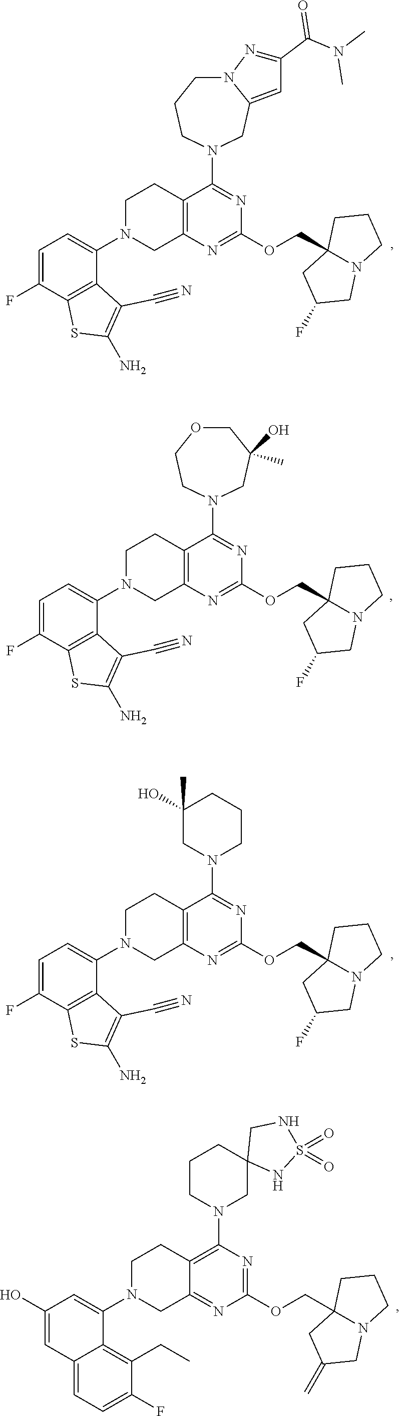 [SMILES: CN(C)C(=O)c1cc2n(n1)CCCN(c1nc(OC[C@@]34CCCN3C[C@H](F)C4)nc3c1CCN(c1ccc(F)c4sc(N)c(C#N)c14)C3)C2 ; C[C@@]1(O)COCCN(c2nc(OC[C@@]34CCCN3C[C@H](F)C4)nc3c2CCN(c2ccc(F)c4sc(N)c(C#N)c24)C3)C1 ; C[C@@]1(O)CCCN(c2nc(OC[C@@]34CCCN3C[C@H](F)C4)nc3c2CCN(c2ccc(F)c4sc(N)c(C#N)c24)C3)C1 ; C=C1CN2CCCC2(COc2nc3c(c(N4CCCC5(CNS(=O)(=O)N5)C4)n2)CCN(c2cc(O)cc4ccc(F)c(CC)c24)C3)C1] 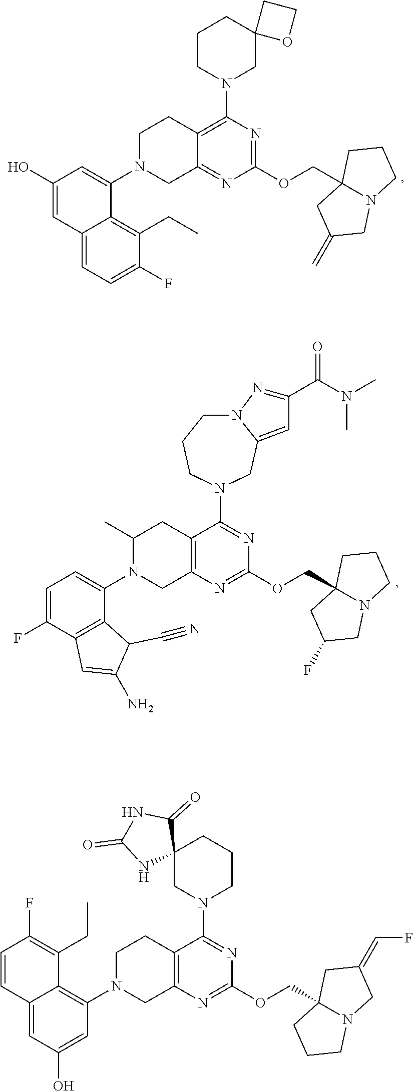 [SMILES: C=C1CN2CCCC2(COc2nc3c(c(N4CCCC5(CCO5)C4)n2)CCN(c2cc(O)cc4ccc(F)c(CC)c24)C3)C1 ; CC1Cc2c(nc(OC[C@@]34CCCN3C[C@H](F)C4)nc2N2CCCn3nc(C(=O)N(C)C)cc3C2)CN1c1ccc(F)c2c1C(C#N)C(N)=C2 ; CCc1c(F)ccc2cc(O)cc(N3CCc4c(nc(OC[C@@]56CCCN5C/C(=C\F)C6)nc4N4CCC[C@]5(C4)NC(=O)NC5=O)C3)c12]
and a pharmaceutically acceptable salt thereof.

62. A pharmaceutical composition comprising a therapeutically effective amount of a compound of any one of claims 1-61 or a pharmaceutically acceptable salt thereof, and a pharmaceutically acceptable excipient.

636. A method for inhibiting wild type KRas or KRas G12A, KRas G12C, KRas G12D, KRas G12R, KRas G12S, KRas G12V, KRas G13D or KRas Q61H activity in a cell, comprising contacting the cell in which inhibition of KRas activity is desired with an effective amount of a compound of according to any one of claims 1-61 or a pharmaceutically acceptable salt thereof, or the pharmaceutical composition of claim 62.

64. A method for treating cancer comprising administering to a patient having cancer a therapeutically effective amount of a compound according to any one of claims 1-61 or a pharmaceutically acceptable salt thereof, or the pharmaceutical composition of claim 62.

65. The method of claim 64, wherein the therapeutically effective amount of the compound is between about 0.01 to 100 mg/kg per day.

66. The method of claim 65, wherein the therapeutically effective amount of the compound is between about 0.1 to 50 mg/kg per day.

67. The method of claim 64, wherein the cancer is selected from the group consisting of cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

68. The method of claim 67, wherein the cancer is a KRas G12A-associated cancer.

69. The method of claim 67, wherein the cancer is a KRas G12C-associated cancer.

70. The method of claim 67, wherein the cancer is a KRas G12D-associated cancer.

71. The method of claim 67, wherein the cancer is a KRas G12R-associated cancer.

72. The method of claim 67, wherein the cancer is a KRas G12S-associated cancer.

73. The method of claim 67, wherein the cancer is a KRas G12V-associated cancer.

74. The method of claim 67, wherein the cancer is a KRas G13D-associated cancer.

75. The method of claim 67, wherein the cancer is a KRas Q61H-associated cancer.

76. The method of claim 67, wherein the cancer is a wild type KRas-associated cancer.

77. The method of claim 67, wherein the cancer is associated with at least one of KRas wild type or KRas G12A, KRas G12C, KRas G12D, KRas G12R, KRas G12S, KRas G12V, KRas G13D or KRas Q61H.

78. The method of any of claims 64-77, wherein the cancer is non-small cell lung cancer, small cell lung cancer, colorectal cancer or pancreatic cancer.

79. A method for treating cancer in a patient in need thereof, the method comprising (a) determining that the cancer is associated with KRas wild type or a KRas G12A, KRas G12C, KRas G12D, KRas G12R, KRas G12S, KRas G12V, KRas G13D or KRas Q61H mutation; and (b) administering to the patient a therapeutically effective amount of a compound according to any one of claims 1-64 or a pharmaceutically acceptable salt thereof, or the pharmaceutical composition of claim 65.

80. The method of any one of claims 64-79, wherein the administering is done via a route selected from the group consisting of parenteral, intraperitoneal, intradermal, intracardiac, intraventricular, intracranial, intracerebrospinal, intrasynovial, intrathecal administration, intramuscular injection, intravitreous injection, intravenous injection, intra-arterial injection, oral, buccal, sublingual, transdermal, topical, intratracheal, intrarectal, subcutaneous, and topical administration.

81. The method of claim 80, wherein the administration route is oral.

82. The method of claim 80, wherein the administration is intravenous injection.

83. The method of claim 80, wherein the administration route is intramuscular injection.

84. The method of claim 80, wherein the administration route utilizes a delivery device.

85. The method of claim 80, wherein administration is done in a hospital setting.
